(19) United States
(12) Patent Application Publication
(10) Pub. No.: US 2006/0257852 A1
(43) Pub. Date: Nov. 16, 2006

(54) Severe acute respiratory syndrome coronavirus

(75) Inventors: Rino Rappuoli, Castelnuovo Berardenga (IT); Vega Masignani, Siena (IT); Konrad Stadler, Scharnstein (AU); Jens Peter Gregersen, Wetter (DE); David Chien, Alamo, CA (US); Jang Han, Lafayette, CA (US); John M. Polo, Danville, CA (US); Amy Weiner, Fairfield, CA (US); Michael Houghton, Danville, CA (US); Hyun Chul Song, Berkeley, CA (US); Mi-Young Seo, Yongin-si (KR); John Donnelly, Moraga, CA (US); Hans Dieter Klenk, Marburg (DE); Nicholas Valiante, Fremont, CA (US)

Correspondence Address: Chiron Corporation;Intellectual Property - R440, P.O. Box 8097, Emeryville, CA, 94662-8097, US

(73) Assignee: Chiron Corporation, Emeryville, CA (US)

(21) Appl. No.: 10/822,303

(22) Filed: Apr. 9, 2004

Related U.S. Application Data
(60) Provisional application No. 60/462,218, filed on Apr. 10, 2003.
(60) Provisional application No. 60/462,465, filed on Apr. 11, 2003.
(60) Provisional application No. 60/462,418, filed on Apr. 12, 2003.
(60) Provisional application No. 60/462,748, filed on Apr. 13, 2003.
(60) Provisional application No. 60/463,109, filed on Apr. 14, 2003.
(60) Provisional application No. 60/463,460, filed on Apr. 15, 2003.
(60) Provisional application No. 60/463,668, filed on Apr. 16, 2003.
(60) Provisional application No. 60/463,983, filed on Apr. 17, 2003.
(60) Provisional application No. 60/463,971, filed on Apr. 18, 2003.
(60) Provisional application No. 60/464,899, filed on Apr. 22, 2003.
(60) Provisional application No. 60/464,838, filed on Apr. 22, 2003.
(60) Provisional application No. 60/465,273, filed on Apr. 23, 2003.
(60) Provisional application No. 60/465,535, filed on Apr. 24, 2003.
(60) Provisional application No. 60/468,312, filed on May 5, 2003.
(60) Provisional application No. 60/473,144, filed on May 22, 2003.
(60) Provisional application No. 60/495,024, filed on Aug. 14, 2003.
(60) Provisional application No. 60/505,652, filed on Sep. 23, 2003.
(60) Provisional application No. 60/510,781, filed on Oct. 11, 2003.
(60) Provisional application No. 60/529,464, filed on Dec. 11, 2003.
(60) Provisional application No. 60/536,177, filed on Jan. 12, 2004.
(60) Provisional application No. 60/560,757, filed on Apr. 7, 2004.

(51) Int. Cl. C12Q 1/70; C07H 21/04; C12N 15/86; C07K 14/165

(52) U.S. Cl. 435/5; 435/69.3; 435/456; 435/325; 530/350; 536/23.72

(57) ABSTRACT

An outbreak of a virulent respiratory virus, now known as Severe Acute Respiratory Syndrome (SARS), was identified in Hong Kong, China and a growing number of countries around the world in 2003. The invention relates to nucleic acids and proteins from the SARS coronavirus. These nucleic acids and proteins can be used in the preparation and manufacture of vaccine formulations, diagnostic reagents, kits, etc. The invention also provides methods for treating SARS by administering small molecule antiviral compounds, as well as methods of identifying potent small molecules for the treatment of SARS.

All documents cited herein are incorporated by reference in their entirety.

### RELATED APPLICATIONS, FROM WHICH PRIORITY IS CLAIMED

This application incorporates by reference in its entirety U.S. provisional patent application 60/462,218, Attorney Reference No. PP20474.001, filed on Apr. 10, 2003 via Express Mail with the US post office, U.S. provisional patent application 60/462,465, Attorney Reference No. PP20480.001, filed on Apr. 11, 2003 via Express Mail with the US post office, U.S. provisional patent application 60/462,418, Attorney Reference No. PP20480.002, filed on Apr. 12, 2003 via Express Mail with the US post office, U.S. provisional patent application 60/462,748, Attorney Reference No. PP20480.003, filed on Apr. 13, 2003 via Express Mail with the US post office, U.S. provisional patent application 60/463,109, Attorney Reference No. PP20480.004, filed on Apr. 14, 2003 via Express Mail with the US post office, U.S. provisional patent application 60/463,460, Attorney Reference No. PP20480.005, filed on Apr. 15, 2003 via Express Mail with the US post office, U.S. provisional patent application 60/463,668, Attorney Reference No. PP20480.006, filed on Apr. 16, 2003 via Express Mail with the US post office, U.S. provisional patent application 60/463,983, Attorney Reference No. PP20480.007, filed on Apr. 17, 2003 via Express Mail with the US post office, U.S. provisional patent application 60/463,971, Attorney Reference No. PP20480.008, filed on Apr. 18, 2003 via Express Mail with the US post office, U.S. provisional patent application 60/464,899, Attorney Reference No. PP20480.009, filed on Apr. 22, 2003 via Express Mail with the US post office, U.S. provisional patent application 60/464,838, Attorney Reference No. PP20507.001, filed on Apr. 22, 2003 via Express Mail with the US post office, U.S. provisional patent application 60/465,273, Attorney Reference No. PP20518.001, filed on Apr. 23, 2003 via Express Mail with the US post office, U.S. provisional patent application 60/465,535, Attorney Reference No. PP20518.002, filed on Apr. 24, 2003 via Express Mail with the US post office, U.S. provisional patent application 60/468,312, Attorney Reference No. PP20480.010, filed on May 5, 2003 via Express Mail with the US post office, and U.S. provisional patent application 60/473,144, Attorney Reference No. PP20480.011, filed on May 22, 2003, U.S. provisional patent application 60/495,024, Attorney Reference No. PP20480.012, filed on Aug. 14, 2003 via Express Mail with the US post office, U.S. provisional patent application 60/505,652, Attorney Reference No. PP20480.013, filed on Sep. 23, 2003 via Express Mail with the US post office, U.S. provisional patent application 60/510,781, Attorney Reference No. PP20480.014, filed on Oct. 11, 2003 via Express Mail with the US post office, U.S. provisional patent application 60/529,464, Attorney Reference No. PP20480.015, filed on Dec. 11, 2003 via Express Mail with the US post office, U.S. provisional patent application 60/536,177, Attorney Reference No. PP20480.016, filed on Jan. 12, 2004 via Express Mail with the US post office, and U.S. provisional patent application 60/______, Attorney Reference No. PP20480.017, filed on Apr. 7, 2004 via Express Mail with the US post office.

### FIELD OF THE INVENTION

The invention relates to nucleic acids and proteins from Severe Acute Respiratory Syndrome (SARS) Virus. These nucleic acids and proteins can be used in the preparation and manufacture of vaccine formulations for the treatment or prevention of SARS. The invention also relates to diagnostic reagents, kits (comprising such reagents) and methods which can be used to diagnose or identify the presence or absence of a SARS virus in a biological sample. The invention also relates to methods for the treatment or prevention of SARS utilizing small molecule viral inhibitors and combinations of small molecule viral inhibitors and kits for the treament of SARS.

### BACKGROUND OF THE INVENTION

An outbreak of a virulent respiratory virus, now known as Severe Acute Respiratory Syndrome (SARS), was identified in Hong Kong, China and a number of other countries around the world in 2003. Patients typically had symptoms including fever, dry cough, dyspnea, headache, and hypoxemia. Isolates of the SARS virus appear to have homology with at least the RNA polymerase gene of several known coronaviruses. A phylogenetic analysis of this homology is presented in Peiris et al., “Coronavirus as a possible cause of severe acute respiratory syndrome”, Lancet, published online Apr. 8, 2003 at http://image.thelancet.com/extras/03art3477web.pdf, incorporated herein by reference in its entirety. Other sequenced fragments of the SARS virus genome appear to overlap with the open reading frame 1b of coronaviruses. See, Drosten et al., “Identification of a Novel Coronavirus in Patients with Severe Acute Respiratory Syndrome”, New England Journal of Medicine, published online at http://www.nejm.org on Apr. 10, 2003, incorporated herein by reference in its entirety.

The Genome Science Center in British Colombia, Canada published on its website (http://www.bcgsc.ca/bioinfo/SARS/) a draft genome assembly of 29,736 base pairs of a virus believed to be a SARS virus, referred to as the TOR2 isolate. This draft genome assembly is given herein as SEQ ID NO: 1.

The Centers for Disease Control (CDC) published a nucleotide sequence of a SARS-CoV strain (SEQ ID NO: 2) on its website (http://www.cdc.gov/ncidod/sars/pdf/nucleoseq.pdj). The CDC has also published a phylogenetic tree of the predicted N, S and M proteins (attached as FIG. 6). This tree places the SARS virus outside any of the previously known coronavirus groups.

There is a growing need for prophylactic or therapeutic vaccines against the SARS virus as well as diagnostic and screening methods and compositions to identify the presence of the virus in, e.g., mammalian tissue or serum.

### SUMMARY OF THE INVENTION

The invention relates to nucleic acids and proteins from Severe Acute Respiratory Syndrome (SARS) virus. These nucleic acids and proteins can be used in the preparation and manufacture of vaccine formulations for the treatment or prevention of SARS. Such vaccine formulations may include an inactivated (or killed) SARS virus, an attenuated SARS virus, a split SARS virus preparation and a recombinant or purified subunit formulation of one or more SARS viral antigens. Expression and delivery of the polynucleotides of the invention may be facilitated via viral vectors and/or viral particles.

The invention also relates to diagnostic reagents, kits (comprising such reagents) and methods which can be used to diagnose or identify the presence or absence of a SARS virus in a biological sample. The invention further includes non-coding SARS viral polynucleotide sequences, SARS viral sequences encoding for non-immunogenic proteins, conserved and variant SARS viral polynucleotide sequences for use in such diagnostic compositions and methods.

The invention further relates to vaccine formulations comprising one or more SARS virus antigens and one or more other respiratory virus antigens. Additional respiratory virus antigens suitable for use in the invention include antigens from influenza virus, human rhinovirus (HRV), parainfluenza virus (PIV), respiratory syncytial virus (RSV), adenovirus, metapneumovirus, and rhinovirus. The additional respiratory virus antigen could also be from a coronavirus other than the SARS coronavirus. Preferably, the additional respiratory virus antigen is an influenza viral antigen.

The compositions of the invention may further comprise one or more adjuvants. Adjuvants suitable for use in the invention include mucosal, transdermal or parenteral adjuvants. Mucosal adjuvants suitable for use in the invention include detoxified bacterial ADP-ribosylating toxins, such as E. coli heat labile toxoids (e.g., LTK63), chitosan and derivatives thereof, and non-toxic double mutant forms of Bordetella pertussis toxoids. Parenteral adjuvants suitable for use in the invention include MF59 and aluminum or aluminum salts.

The invention also provides methods for treating SARS by administering small molecule compounds, as well as methods of identifying potent small molecules for the treatment of SARS.

In one aspect of the invention a method of identifying a therapeutically active agent is provided comprising: (a) contacting the therapeutically active agent with a cell infected with the SARS virus; (b) measuring attenuation of a SARS related enzyme.

In a more particular embodiment, the therapeutically active agent is a small molecule. In another more particular embodiment, the therapeutically active agent is a nucleoside analog. In another more particular embodiment the therapeutically active agent is a peptoid, oligopeptide, or polypeptide. In another embodiment the SARS related enzyme is SARS protease. In another embodiment the SARS related enzyme is SARS polymerase. In still another embodiment the SARS related enzyme is a kinase. Methods of identifying therapeutically active agents for treatment of SARS virus infection are further discussed in Section V below.

In another aspect of the invention a method of treating a human infected with SARS is provided comprising administering a small molecule to a patient in need thereof. In one embodiment the small molecule is an inhibitor of SARS protease. In another embodiment the small molecule is an inhibitor of SARS polymerase. In another embodiment the SARS related enzyme is a kinase. In still another embodiment the small molecule is administered orally or parenterally.

The invention also provides the use of such small molecules in the manufacture of a medicament for the treatment of severe acute respiratory syndrome.

Small molecule compounds of the present invention include those of less than 1000 g/mol, preferably with an aromatic region and greater than one heteroatom selected from O, S, or N. Preferred small molecules include, but are not limited to acyclovir, gancyclovir, vidarabidine, foscamet, cidofovir, amantidine, ribavirin, trifluorothymidine, zidovudine, didanosine, zalcitabine, and combinations thereof. Interferons may also be used for treating patients, including interferon-α and interferon-β. Interferon treatment has shown promise in treating SARS in monkeys (Enserink (2004) Science 303:1273-1275), particularly when pegylated (Haagmans et al. (2004) Nature Medicine 10:290-293).

One aspect of the present invention relates to methods for identifying individuals exposed to, and biological samples containing SARS virus (SARSV), and to kits for carrying out the methods. Such methods can utilize nucleic acid detection techniques such as PCR, RT-PCR (the Coronaviridae are RNA viruses), transcription-mediated amplification (TMA), ligase chain reaction (LCR), branched DNA signal amplification assays, isothermal nucleic acid sequence based amplification (NASBA), other self-sustained sequence replication assays, boomerang DNA amplification, strand-displacement activation, cycling probe technology, or combinations of such amplification methods. Such nucleic acid detection techniques utilize oligonucleotides having nucleotide sequence similar to, or complementary to, the SARS viral genome, as primers (e.g., for amplification) and as probes (e.g., for capture or detection), as is well known in the art.

Alternatively, or in addition to the nucleic acid detection methods described supra, the methods of the present invention can utilize various immunoassay techniques for detection of SARSV antigens and/or antibodies.

Accordingly, the present invention relates to methods of identifying individuals exposed to SARSV, or biological samples containing SARSV, by detecting the presence of SARSV antigens using antibodies which specifically bind to the same. The antibodies are preferably monoclonal antibodies. Quantification of the amount of viral antigens present in a sample of an individual may be used in determining the prognosis of an infected individual. Preferably, the SARSV antigens to be detected are generally one of the structural proteins, particularly those present on the surface of the viral particles and include, for example, the spike glycoprotein (S), also called E2; the envelope (small membrane) protein (E), also called sM; the membrane glycoprotein (M), also called E1 ; the hemagglutinin-esterase glycoprotein (HE); also called E3; and the nucleocapsid phosphoprotein (N). In preferred embodiments, the antigens to be detected are the S, E and M proteins using antibodies to the same.

The present invention relates to kits for identifying individual SARSV and reagents used in such kits. The kits comprise a first container which contains antibodies which specifically bind to a SARSV antigen and a second container which contains the SARSV antigen. The antibodies are preferably monoclonal antibodies. The kits may be adapted for quantifying the amount of antigen in a sample of an individual. Such information may be used in determining the prognosis of an infected individual.

The present invention relates to methods of identifying individuals exposed to SARS virus, or biological samples containing SARSV, by detecting the presence of antibodies against SARS virus antigen in a sample using SARS antigen. Quantification of the amount of anti-SARS protein from SARS antibodies present in a sample of an individual may be used in determining the prognosis of an infected individual. Any one or more of the viral proteins (structural proteins or nonstructural proteins) may be used as antigen to detect the SARSV antibodies; preferably a SARSV antigen that is conserved amoung SARSV isolates is preferred. In this regard, nonstructural protein (e.g., Pol, Hel, 3CLp, MP, PLP1, PLP2) may be particularly useful.

The present invention relates to kits for identifying individuals exposed to SARS and reagents used therein. The kits comprise a first container which contains antibodies which were produced in response to exposure to an antigen from SARS virus and a second container which contains the SARS antigen(s). The kits may be adapted for quantifying the amount of anti-SARS antibodies present in a sample of an individual. Such information may be used in determining the prognosis of an infected individual.

The present invention relates to methods of identifying individuals exposed to SARS virus, or biological samples containing SARSV, by detecting the presence of nucleic acid from SARS virus. Quantification of the amount of SARS nucleic acid present in a sample of an individual may be used in determining the prognosis of an infected individual. The methods utilize oligonucleotide probes and/or primers that are similar or complementary in sequence to the SARSV genome or transcription or replication products. Preferred probes and primers are described herein. Also included in the present invention are kits for carrying out the methods of detecting the SARSV nucleic acid.

The invention further includes a method for the treatment and/or prevention of SARS through the administration of a therapeutically effective amount of at least one antiviral compound from among those described in the US patents and published international patent applications listed in Table 1 and Table 2. In one embodiment of the method, the antiviral compound is a small molecule. In another embodiment, the antiviral compound is a protease inhibitor. In a further embodiment, the antiviral protease inhibitor is a 3C-like protease inhibitor and/or a papain-like protease inhibitor. In another embodiment, the antiviral compound is an inhibitor of an RNA-dependent RNA polymerase. In another embodiment, a first antiviral compound which is a protease inhibitor is administered with a second antiviral compound which is an RNA-dependent RNA polymerase inhibitor. The invention further provides for the administration of a steroidal anti-inflammatory drug in combination with at least one antiviral compound, for example, from the antiviral compounds described in the documents listed in Table 1 and Table 2.

The invention further provides for a method for the treatment and/or prevention of SARS through the administration of a therapeutically effective amount of at least one antiviral compound from among those described in the US patents and published international patent applications listed in Table 1 and Table 2 by inhalation. In one embodiment of the method, the antiviral compound is a small molecule. In another embodiment, the antiviral compound is a protease inhibitor. In a further embodiment, the antiviral protease inhibitor is a 3C-like protease inhibitor and/or a papain-like protease inhibitor. In another embodiment, the antiviral compound is an inhibitor of an RNA dependent RNA polymerase. In another embodiment, a first antiviral compound which is a protease inhibitor is administered with a second antiviral compound which is an RNA-dependent RNA polymerase inhibitor. The invention further provides for the administration of a steroidal anti-inflammatory drug in combination with at least one antiviral compound, for example, from the antiviral compounds described in the documents listed in Table 1 and Table 2 by inhalation. The steroidal anti-inflammatory drug may be administered by inhalation for a local effect or administered for systemic absorption such as via an oral or intravenous route.

The invention further provides the use of an antiviral compound, as defined above, in the manufacture of a medicament for the treatment of severe acute respiratory syndrome.

The invention further provides for a kit for use by a consumer for the treatment and/or prevention of SARS. Such a kit comprises: (a) a pharmaceutical composition comprising a therapeutically effective amount of at least one antiviral compound from among those described in the US patents and published international patent applications listed in Table 1 and Table 2 and a pharmaceutically acceptable carrier, vehicle or diluent; (b) a container for holding the pharmaceutical composition; and, optionally; (c) instructions describing a method of using the pharmaceutical compositions for the treatment and or the prevention of SARS. The kit may optionally contain a plurality of antiviral compounds for the treatment of SARS wherein the anti viral compounds are selected from 3C-like protease inhibitors and papain-like protease inhibitors. In a further embodiment, the kit contains an antiviral compound which is an RNA-dependent RNA polymerase inhibitor. When the kit comprises more than one antiviral compound, the antiviral compounds contained in the kit may be optionally combined in the same pharmaceutical composition.

An additional aspect of the invention provides for the use of at least one of the antiviral compounds described in the US patents and published international patent applications listed in Table 1 and Table 2 for the manufacture of a medicament for the treatment or prevention of SARS.

### BRIEF DESCRIPTION OF THE DRAWINGS

FIG. 1: Schematic of coronavirus genome organization.

FIG. 2: Schematic of coronavirus ORF1a/ORF1b gene products.

FIG. 3(A-C): Alignment of coronavirus polynucleotide sequences for selected genes (including nucleocapsid (N), matrix (M), and hemagluttinin-esterase (HE)).

FIG. 4(A-F): Alignment of coronavirus polypeptide sequences (including ORF1a/ORF1b, nucleocapsid (NP), hemagluttinin-esterase (HE), envelope (Sm or E), matrix (M), and spike (S).

FIG. 5: Alignment of spike (S) polypeptide sequences, taken from FIG. 4, in the region of the junction of the S1 and the S2 domains, and protease cleavage site for selected coronaviruses.

FIG. 6: CDC phylogenetic tree of SARS-CoV strain (Clustalx 1.82, neighbor-joining tree).

FIG. 6A shows coronavirus N protein analysis,

FIG. 6B shows coronavirus S protein analysis, and

FIG. 6C shows coronavirus M protein analysis.

FIG. 7: Conserved and specific sequence of the SARS virus.

FIGS. 7A-7D show multiple sequence alignments (CLUSTAL W 1.82) of the structural proteins of the SARS virus genome (7A: PEP4 Spike protein; 7B: PEP7 small membrane protein; 7C: PEP8 matrix glycoprotein; 7D: PEP13 nucleocapsid protein), which have counterparts in all or some of the other known coronaviruses.

FIGS. 7E-7H show dendrograms reporting the protein distances among the sequences in alignments 7A-7D. Labels 229E: human coronavirus; MEV: murine hepatitis virus; TGV: transmissible gastroenteritis virus; AIBV: avian infectious bronchitis virus; BOVINE: Bovine coronavirus; PEDV: porcine epidemic diarrhea virus.

FIG. 8: Alignment of the 5′UTR of several coronaviruses, to show consensus nucleotide sequence at the 5′UTR.

FIG. 9: Sequences of preferred primers for amplification of the 5′UTR. F and R denote forward and reverse PCR primers, and the numbers indicate nucleotide positions withing FIG. 8.

FIG. 10: Alignment of the 3′UTR of several coronaviruses, to show consensus nucleotide sequence at the 3′UTR.

FIG. 11: Sequences of preferred primers for amplification of the 3′UTR. F and R denote forward and reverse PCR primers, and numbers indicate nucleotide positions within FIG. 10.

FIG. 12: Coiled-coil prediction for SEQ ID NO: 6042, using Coils program (FIG. 12A) or LearrCoil (FIG. 12B).

FIG. 13: Example of insertion of a reporter gene-of-interest at a site between exisiting SARS virus genes. Small nonstructural gene products are not depicted schematically.

FIG. 14: Schematic depicting representative examples of SARS virus replicons. Small nonstructural gene products are not depicted schematically.

FIG. 15: SARS virus nsp2 proteinase (3CLp) and identification of catalytic and substrate sites.

FIG. 16: alignment of SARS virus nsp2 proteinase (3CLp) with that of avian IBV, MHV, and BCoV. Residues in dotted boxes are key residues the substrate sites (F, Y & H); residues in solid boxes are catalytic cysteine (C) and histidine (H) residues.

FIG. 17: Genome organization of SARS coronavirus. Replicase and structural regions are shown, along with the predicted products of cleavage within ORF1a and ORF1b. The position of the 5′ RNA leader sequence (L), the 3′ poly(A) tract and the ribosomal frame-shift consensus between ORF1a and ORF1b are also indicated. Each box represent a protein product. They are shaded according to the level of amino acid identity with corresponding proteins of other coronaviruses (see also Table 2). The SARS-specific genes are white. Positions of the 9 SARS-specific six-base IG sequences (5′-ACGAAC-3′; SEQ ID NO 7293) are indicated by arrows.

FIG. 18: Genome organization of Coronaviruses representative of group 1 (HCoV-229E, accession number: AF304460), group 2 (mouse hepatitis virus MHV, accession number: NC_{—}001846), group 3 (avian infectious bronchitis virus AIBV, accession number: NC_{—}001451) and SARS coronavirus. Other completely sequenced coronaviruses used in this study are available at the following accession numbers: porcine epidemic diarrhea virus (PEDV), AF353511; transmissible gastroenteritis virus (TGV), ND_{—}002306; Bovine coronavirus (BCoV): AF220295. Red boxes represent group-specific genes. The position of the leader RNA sequence and poly(A) tract is also indicated in genomes where they are reported. The position of specific IG sequences is indicated by circles of different shades. In the SARS genome, we also find three IG sequences specific for group 2 coronavirus.

FIG. 19: Topological model predicted for the spike protein anchored to the viral membrane. Structural and predicted functional domains are indicated. The N-terminal region (S1) is predicted to contain the receptor binding domain. Two coiled coil regions within the S2 domain, partially superimposed to leucine zipper motifs are presumably involved in oligomerization. The hydrophobic domain is responsible for membrane anchoring.

FIG. 20: Phylogenetic tree obtained from the multiple sequence alignment of a 922 bp internal region of the pol gene from 12 coronaviruses and SARS. Numbers at the nodes represent the result of a bootstrap analysis and strongly support the branches. Sequences not available within the complete coronavirus genomes have been retrieved from GenBank at the following accession numbers: hemagglutinating encephalomyelitis virus of swine (PHEV), AF124988, Human OC43 virus (OC43), AF124989, canine coronavirus (CCV), AF124986, feline infectious peritonitis virus (FIPV), AF124987, turkey coronavirus (TCV), AF124991, syaloacryoadenitis virus of rats (SDAV), AF124990.

FIG. 21: 21A. Unrooted tree obtained from the alignment of consensus sequences of the group I and group II S1 domain of spike proteins (G1_cons and G2_cons) with those of a group 3 spike (AIBV) and the spike of SARS virus. The number indicates the result of a bootstrap analysis. The sequences used to generate the consensus profile from group 1 are: HcoV-229E, accession number P15423; porcine epidemic diarrhea virus (PEDV), acc no: NP_{—}598310; transmissible gastroenteritis virus (TGV), acc no: NP_{—}058424; Canine coronavirus (CCV), acc no: S41453; porcine respiratory virus (PRV), acc no: S24284; feline infectious peritonitis virus (FIPV), acc no: VGIH79. The sequences used to generate the consensus profile from group 2 are: mouse hepatitis virus (MHV), acc no: NP_{—}045300; Bovine coronavirus (BCoV), acc no: NP_{—}150077; Human coronavirus OC43, acc no: P36334; hemagglutinating encephalomyelitis virus of swine (PHEV), acc no: AAL80031; for group 3, only the sequence of the spike protein of avian infectious bronchitis virus (AIBV), acc no: AAO34396 was used. 21B: Schematic representation of cysteine positions in S1 domains of group 1, 2 and 3, compared to the SARS spike. Horizontal bars represent the S1 amino acid sequences (in the case of SARS and AIBV) or the consensus profiles (generated from group 1, G1 _cons, and from group 2, G2_cons). The length of the bars are not to scale. Relative cysteine positions are indicated by rectangle bars. Only cysteines perfectly conserved within each consensus are reported. Lines connect cysteines conserved between the SARS S1 domain and the consensus sequences as shown.

FIG. 22: illustration of a Neisseria Adhesin A protein (NadA).

FIG. 23: Raw translation from SARS coronavirus genome (reading frame +1).

FIG. 24: Raw translation from SARS coronavirus genome (reading frame +3)

FIG. 25: 1b and Spike open reading frames, separated by *.

FIG. 26: SARS growth in vero cells.

FIG. 27: chromatogram of the capture step of SARS coronavirus on Matrix Cellufine Sulfate Superformance 150/10. Analysis was on 100 ml coronoavirus harvest. The left Y axis shows absorbance at 280 nm. The right Y axis shows the gradient (% B). The X axis shows the volume (ml).

FIG. 28: Silver-stained MCS chromatography fractions. Lanes are: (1) marker; (2) coronavirus vero cell harvest; (3) coronavirus vero cell harvest, after 0.65 μm filtration; (4) flowthrough; (5) wash; (6) 20% peak (virus peak). Lanes were loaded with 1 μg of test protein.

FIG. 29: Western Blot of MCS chromatography fractions. Lanes are as described for FIG. 28.

FIG. 30: Linear density gradient ultracentrifugation, 15-60% sucrose (SW28, 2 hours, 20000 rpm). The graph shows protein concentration (▪) and sucrose concentration (♦).

FIG. 31: Silver-stained density gradient fractions on NuPage 4-12% Bis-Tris-Ge (Novex), reduced conditions, heated for 10 minutes at 70° C. Lanes are: (1) marker; (2) 20% peak MCS; (3) density gradient fraction 11; (4) density gradient fraction 12; (5) density gradient fraction 13; (6) density gradient fraction 14; (7) density gradient fraction 15; (8) density gradient fraction 16; (9) density gradient fraction 17. The bulk of proteins was in fractions 15 to 17. Lanes 2, 8 and 9 were loaded with 1 μg protein.

FIG. 32: Chromatogram of the Capture Step of SARS coronavirus on MCS. Details are as for FIG. 27, except that 200 ml harvest was used.

FIG. 33: Silverstain (left) and Western Blot (right) of chromatographic fractions. Lanes are as described for FIGS. 28 and 29, except that lane (6) is the 5% peak. Treatment before SDS-PAGE was at room temperature for 30 minutes.

FIG. 34: Density Gradient Ultracentrifugation, 15-40% sucrose (SW28, 2 hours, 20000 rpm). The graph shows protein concentration (▪) and sucrose concentration (♦).

FIG. 35: Silverstain (left) and Western Blot (right) of Density Gradient Ultracentrifugation fractions on NuPage 4-12% Bis-Tris-Ge (Novex), reduced conditions. Lanes are: (1) marker; (2) density gradient fraction 6; (3) density gradient fraction 7; (4) density gradient fraction 8; (5) density gradient fraction 9; (6) density gradient fraction 10; (7) density gradient fraction 15. Fractions 7-10 (lanes 3-6) contained pure coronavirus proteins. The bulk of impurities was in fraction 15 (lane 7). Lanes 2, 8 and 9 were loaded with ˜1 μg protein. Treatment before SDS-PAGE was at room temperature for 30 minutes.

FIG. 36: EM pictures of Density Gradient Fractions 8-10. FIG. 36A shows fraction 8; FIG. 36B shows fraction 9; FIG. 36C shows fraction 10.

FIG. 37: Spike/NadA fusion constructs.

FIGS. 38 and 39: Results of the expression in E. coli of S1_{L}, S1_{L}-NadA and S1_{L}-NadA_{Δanchor}.

FIG. 38 shows SDS-PAGE analysis of total lysates from BL21(DE3)/pET, BL21(DE3)/pET-S1_{L} and BL21 (DE3)/pET-S1_{L}-NadA_{Δanchor}. The bands are indicated by an arrow, and the three lanes are, from left to right: BL21(DE3)/pET; BL21(DE3)/pET-S1_{L}; BL21(DE3)/pET-S1_{L}-NadA_{Δanchor}.

FIG. 39 shows (39A) SDS-PAGE and (39B) western blot analyses of total lysates from BL21(DE3)/pET, BL21(DE3)/pET-S1_{L}-NadA (grown under un-induced condition) and BL21(DE3)/pET-S1_{L}-NadA (grown under induced condition). The bands are indicated by an arrow, and lanes are, from left to right: BL21(DE3)/pET; BL21(DE3)/pET-S1_{L}-NadA; BL21(DE3)/pET-S1_{L}-NadA. The western blot shows the presence of oligomeric forms of the protein.

FIG. 40: Schematic of SARS Spike clones.

FIG. 41: Transient Expression of SARS Spike Proteins (western blot of COS7 cell lysate). Each lane of the 4-20% TG SDS gel was loaded with 20 μg cell lysate (total 1.2 mg). The labeling antibodies are shown.

FIG. 42: Western blot analyses of COS7 cell lysates on 4% TG SDS gel showing oligomerization state of intracellular S molecules.

FIG. 43: Western blot analyses of COS7 cell lysates on 4-20% TG SDS gel showing Transient Expression of SARS Spike Proteins. Lanes are: (1) mock, AF; (2) mock, DF; (3) nSh, AF; (4) nSh, DF; (5) nShΔTC, AF; (6) nShΔTC, DF. Each lane was loaded with 5 μl of each sample, 400 μl total. The blot was labeled with antibody against the His-tagged protein.

FIG. 44: Western blot analyses of COS7 cell medium on 4-20% TG SDS gel showing Transient Expression of SARS Spike Proteins. Truncated spike protein is secreted. Spike proteins were purified from the culture medium (from a 10 cm plate), first by a ConA column and then finally by His·tag Magnetic beads. Each lane was loaded with one third of the material.

FIG. 45: Western blot analyses of COS7 cell lysates on 4-20% TG SDS gel showing glycoslation of SARS spike proteins. In the two left-hand blots (lanes 1-5), samples were boiled in SDS and β-mercaptoethanol; in the two right-hand blots (lanes 6-11), samples were in SDS only, with no boiling. Lanes 1-8 were labeled with a monoclonal raised against the His-tag protein; labes 9-11 were labeled with rabbit anti-SARS antibody.

FIG. 46: Effect of SARS spike protein expression on cell viability.

FIG. 47: Western blot analyses of COS7 cell lysates on 4% TG SDS gels showing oligomerization state of intracellular spike molecules. Blots were labeled with anti-His-tag mAb. The membrane fraction of COS7 cell lysate was fractionated by a sizing column before loading the lanes. Fractions 7 to 14 show bands with kDa values of: 71000, 1400, 898, 572, 365, 232, 148 and 99, respectively.

FIG. 48: Fractionation of cells into aqueous and detergent fractions.

FIG. 49: Schematic of constructs for use in OMV preparation.

FIG. 50: SARS HR1 and HR2 constructs.

FIG. 51: Vaccine protection froms SARS in Balb/c mouse model.

FIG. 52: Expressed on Spike protein in transfected 293 cell lysates (52A) or COS7 cell culture supernatants (52B). Proteins were separated on 4-20% TG SDS gels. The label was anti-His-tag, except for the right-hand three lanes of 52B, where the label was rabbit anti-SARS serum.

In FIG. 52A, the left-hand three lanes were treated with DTT and were boiled, but neither treatment was used for the right-hand three lanes. In FIG. 52B, no DTT was used, but all lanes were heated to 80° C. for 5 minutes.

FIG. 53: Western blot of Spike proteins expressed in COS7 cells. Proteins were incubated at room temperature (RT), 80° C. or 100° C. to check for any effect on molecular weight. FIG. 54 shows similar experiments on SARS virions.

FIG. 55: Results of a pulse chase experiment, showing expression and processing of SARS spike protein following infection with alphavirus replicon particles. Cells were treated with or without EndoH as shown.

FIG. 56: Effect of heating on Spike protein trimers.

FIG. 57: Coomassie blue-stained gel of yeast-expressed proteins. Lanes are: 1-See Blue Standard (10 μl); 2-pAB24 gbl (20 μg); 3-SARS Spike S1 c.1 gbl (20 μg); 4-SARS Spike S1 c.2 gbl (20 μg); 5-See Blue Standard (10 μl ); 6-pAB24 ip (5 μl ); 7-SARS Spike S1 c.1 (5 μl ); 8-SARS Spike S1 c.2 (5 μl).

FIGS. 58 to 64: Schematics of preparation of yeast expression constructs.

FIGS. 65 to 66: Yeast-expressed sequences for Spike.

FIG. 67: Western blots showing expression of SARS spike protein from alphavirus replicon particles and replicon RNA.

FIG. 67A was run under non-reducing conditions and at room temperature (i.e. no heating), with lanes: (1) VEE/SIN-spike infection; (2) VEE/SIN-GFP infection; (3) Replicon-spike RNA transfection; (4) Replicon-GFP RNA transfection.

FIG. 67B was run with SARS virions at different temperatures, as shown.

FIG. 68: induction of antibody responses in mice. Vaccine groups are: (1) Inactivated SARS Virus; (2) Truncated Recombinant Spike Protein; (3) Full length Spike: DNA+DNA.PLG+ Alphavirus; (4) Full length Spike: Alphavirus particles only.

FIG. 69: Binding of human monoclonal antibody S3.2 to purified truncated Spike protein. The X-axis shows antibody concentration, and the Y-axis shows ELISA absorbance. The interpolation result is 2158.13.

FIG. 70: Geometric mean ELISA titers of antibodies induced by the SARS-CoV spike protein delivered as different vaccines (left to right: inactivated virus; 3 μg truncated spike protein; 75 μg DNA encoding truncated spike protein.

FIG. 71: Neutralization titers after immunization with (left) nSdΔTC protein or (right) DNA encoding nSdΔTC, delivered on PLG.

FIG. 72: Correlation between the spike antigen binding and neutralizing antibodies

FIG. 73: Western blot of CHO cell lines expressing Spike protein in full-length form (left) or in truncated form (right). Proteins were separated by 4-12% SDS-PAGE, with boiling in DTT and staining by polyclonal serum.

FIG. 74: Structural components of SARS-CoV spike glycoprotein and expression construct. L denotes leader peptide (residues 1-13), TM the transmembrane, and Cy the cytoplasmic tail segments. The hexa-His tags are not shown.

FIG. 75: Western blot analysis of SARS spike proteins expressed in COS7 cells.

In FIG. 75A, COS7 cells were transfected with indicated plasmid constructs and the expressed proteins in cell lysates 48 hr post-transfection were analysed by SDS-PAGE (4-20% polyacrylamide) in reducing and denaturing condtions, with proteins visualized by anti-histidine Mab.

In FIG. 75B, proteins were collected from cell culture medium 48 hr post-transfection and purified first by a ConA column and then by His-tag magnetic beads. Purifed proteins were analysed by SDS-PAGE (4-20% polyacrylamide) and were visualized by anti-SARS rabbit serum.

FIG. 76: Endo H sensitivity of C-terminal truncated spike protein (SΔ) found in cell lysate (lanes 1,2) and culture medium (lanes 3,4). Positions of internal SΔ protein and secreted SΔ protein are marked with arrow heads.

FIG. 77: Oligomeric status of the SARS spike protein. Recombinant S protein oligomer in COS7 cells transfected with the full-length spike construct (nSh). The cell lysates were treated with DTT and/or heat as indicated above each lane. The different forms of S protein in treated and untreated samples were visualized by SDS-PAGE (4% polyacrylamide) and Western blot analysis using anti-histidine MAb.

FIG. 78: Effect of heat denaturation on the oligomeric status of recombinant S protein in the absence of DTT. The COS7 cell lysates were heated before the electrophoresis as indicated and the S proteins were visualized as described fogi FIG. 77.

FIG. 79: Effect of heat denaturation on the oligomeric status of spike protein in SARS virion particles. SARS-CoV were grown in Vero cells, purified and solubilized from the virion particles by SDS, heat-denatured as indicated and visualized as described in FIG. 77, except that rabbit antiserum against the purified virus was used as a probe.

FIG. 80: Analysis of the oligomeric status of SARS virion spike protein by cross-linking experiment. Solubilized SARS virion proteins were treated with DMS. Both untreated (−) and DMS treated (+) virion proteins were heat denatured in the absence of DTT and visualized by 4% PAGE followed by silver staining.

FIGS. 81 & 82: Analysis of the oligomeric status of truncated spike protein by heat denaturation. Truncated spike protein within COS7 cell lysates (81) or secreted into culture medium (82) were heat denatured as indicated in the absence of DTT and visualized by Western blot analysis.

FIG. 83: Reactivity of deglycosylated full-length spike oligomer with conformational and non-conformational antibody. The full-length recombinant spike oligomer was partially deglycosylated with PNGase F in non denaturating condition and visualized by Western blot analysis using anti-histidine Mab (lane 1,2,3) or rabbit antiserum against purified SARS CoV (lane 4,5,6).

FIG. 84: Localization of expressed SARS spike proteins in fractionated COS7 cell lysate visualized by western blot. Cells were transfected with indicated plasmids and lysed with Dounce homogeniser in hypotonic buffer 48 hr post transfection. Cell lysate was centrifuged to obtain soluble cytosol and insoluble membrane fraction that was further solublized by 4% Triton X-100. Proteins were heated with SDS at 80 C and analysed by SDS-PAGE (4-20% polyacrylamide) in reducing condtion. Proteins were visualized by anti-histidine Mab. The cytosol fractions were loaded in lanes 1, 3, and 5 and the membrane fractions were loaded in lanes 2, 4, and 6.

FIG. 85: Intracellular and surface expression of recombinant full-length (A,D) or truncated (B,E) spike protein in COS7 cells. The cells were fixed at 48 hrs posttransfection and either treated with detergent (Cytofix/perm, BD Biosciences) for intracellular immunofluorescence (A,B,C) or with 2% paraformaldehyde for cell surface immunofluorescence observation (D,E,F) at ×40 magnification. Mock transfected cells (C,F) were included as controls.

FIGS. 86-105: SDS-PAGE od E. coli expressed proteins. Tot=total protein; Sol=soluble protein fraction. Labels are protein names (Tables 26-30).

FIG. 106: Immunofluorescence after administration of vector encoding optimsed N antigen.

FIG. 107: Immunofluorescence of (A) native and (B) codon-optimsed M sequences.

FIG. 108: Immunofluorescence of (A) native and (B) codon optimsed E sequences.

FIGS. 109-111: Western blots of Vero cells using rabbit antibodies obtained after immunization with spike proteins expressed in E. coli.

FIG. 112: Spike protein expression in 293 cells. Lanes: (M) Markers; (1) Mock transfected; (2,6) cells expressing nS protein, lysate; (3,7) cells expressing nSdTC protein, lysate; (4,8) cells expressing nS protein, supernatant; (5,9) (4) cells expressing nSdTC protein, supernatant. Staining antibody: (2 to 5) mouse serum obtained after DNA immunization; (6 to 9) rabbit serum obtained after immunization with whole killed virus.

FIG. 113: Six reading frames of SEQ ID NO: 9968.

FIG. 114: Six reading frames of SEQ ID NO: 10033.

FIG. 115: Alignment of bovine coronavirus pol 1ab (top row; SEQ ID NO: 10068), avian infectious bronchitis pol 1ab (second row; SEQ ID NO: 10069), murine hepatitis virus pol 1ab (third row; SEQ ID NO: 10070), SEQ ID NO^{S}: 9997/9998 (fourth row) and a consensus sequence (bottom row; SEQ ID NO: 10071).

FIG. 116: Schematic of coronavirus genome organization.

FIG. 117: Schematic of coronavirus ORF1a/ORF1b gene products, including “*” region.

FIG. 118: Alignment.

FIG. 119: Alternative start codons within SEQ ID NO: 10080.

FIG. 120: Six reading frames of SEQ ID NO: 10084.

FIG. 121: Alignment of SEQ ID NO: 10033 and SEQ ID NO: 10084.

FIG. 122: Reading frames in SEQ ID NO: 10084.

FIG. 123: Start codon analysis for SEQ ID NO: 10084.

FIG. 124: BLAST analysis of SEQ ID NO: 10210.

FIG. 125: Epitope analysis of SEQ ID NO: 10210 by either (13A) Hopp & Woods or (13B) Kyte & Doolittle.

FIG. 126: Reading frames in SEQ ID NO: 10299.

FIG. 127: Reading frames in SEQ ID NO: 10505.

FIG. 128: Reading frames in SEQ ID NO: 11563.

FIG. 129: Reading frames in SEQ ID NO: 10033.

FIG. 130: Alignment of SEQ ID NO: 9997 and SEQ ID NO: 10033.

FIG. 131: Reading frames in SEQ ID NO: 10299.

FIG. 132: Reading frames in SEQ ID NO: 10505.

FIG. 133: Western Blot of SARS protease purification fractions.

FIG. 134: Cleavage of DABCYL-EDANS (a fluorescent tagged peptide with a SARS protease cleavage site) by SARS protease at different concentrations. The graph shows activity/concentration correlations with no protease (♦), 0.95 uM protease (▪) and 2.86 uM protease (●).

In the event of a discrepancy between a sequence in the sequence listing and a sequence in the drawings, the drawings should take precedence.

### DETAILED DESCRIPTION OF THE INVENTION

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, Mack Publishing Company, Easton, Pa., 19th Edition (1995); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Handbook of Surface and Colloidal Chemistry (Birdi, K. S. ed., CRC Press, 1997); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Peters and Dalrymple, Fields Virology (2d ed), Fields et al. (eds.), B.N. Raven Press, New York, N.Y.

All publications, patents and patent applications cited herein, are hereby incorporated by reference in their entireties.

Severe Acute Respiratory Syndrome (SARS) virus has recently been identified as a new viral species. The SARS viral species includes the following isolates.
two virus isolates described in Peiris et al. “Coronavirus as a possible cause of severe acute respiratory syndrome” Lancet published online at http://image.thelancet.com/extras/03art3477web.pdf on Apr. 8 2003, incorporated herein by reference in its entirety and the sequences deposited with GenBank at accession number AY268070.
the isolates and viral sequences described in Drosten et al., “Identification of a Novel Coronavirus in Patients with Severe Acute Respiratory Syndrome”, New England Journal of Medicine, published online at http://www.nejm.org on Apr. 10, 2003.
the isolates and viral sequences described on the website of the WHO network on Mar. 25 and 24, 2003.
the isolates and viral sequences described in Tsang et al., “A Cluster of Cases of Severe Acute Respiratory Syndrome in Hong Kong” New England Journal of Medicine, published online at http://www.nejm.org on Mar. 31, 2003.
the isolates and viral sequences described in Poutanen et al., “Identification of Severe Acute Respiratory Syndrome in Canada” New England Journal of Medicine, published online at http://www.nejm.org on Mar. 31, 2003.
   As described in the Lancet article, a 646 base pair polynucleotide from the SARS virus has weak homology to viruses of the family Cornoaviridae. The Lancet article further reports that a deduced amino acid sequence (of 215 amino acids) from this sequence has about 57% sequence homology to the RNA polymerase of bovine coronavirus and murine hepatitis virus. Phylogenetic analysis of the protein sequences are also presented in the Lancet article showing that the polymerase sequence is most closely related to the group II coronaviruses.

Additional SARS viral isolates can be identified, isolated and/or sequenced by virologists skilled in the art. Virologists can readily identify new viral isolates as a SARS virus. Criteria which a virologist may use to identify new SARS isolates include: sequence homology of the new isolate to known SARS viral isolates; similar genomic organization of the new viral isolate to known SARS viral isolates; immunological (serologic) similarity or identity with known SARS viral isolates; pathology; and similarity of virion morphology with known SARS viral isolates; and similarity of infected cell morphology as that caused by known SARS viral isolates (visualized, for instance, by electron microscopy).

Methods for isolating and sequencing SARS viral isolates include the methods described by Peiris et al. in the Lancet paper. As reported in the Lancet paper, RNA from clinical samples can be reverse transcribed with random hexamers and cDNA can be amplified with primers having sequences of SEQ ID NOS: 6584 & 6585 in the presence of 2.5 mmol/L magnesium chloride (94° C. for 1 min, 50° C. for 1 min, and 72° C. for 1 min).

Reverse transcription of a viral isolate using random hexamers can be accomplished in an RT-PCR assay as follows. Virus isolates are propagated on mammalian cells, particularly fetal rhesus kidney cells. Total RNA from virus-infected and virus-uninfected fetal rhesus kidney cells is then isolated. RNA samples are reverse transcribed with a primer having SEQ ID NO: 6586. cDNA can be amplified by a primer having SEQ ID NO: 6587. Unique PCR products (in size) in the infected cell preparation are then cloned and sequenced, and genetic homology of the sequence compared with those in GenBank.

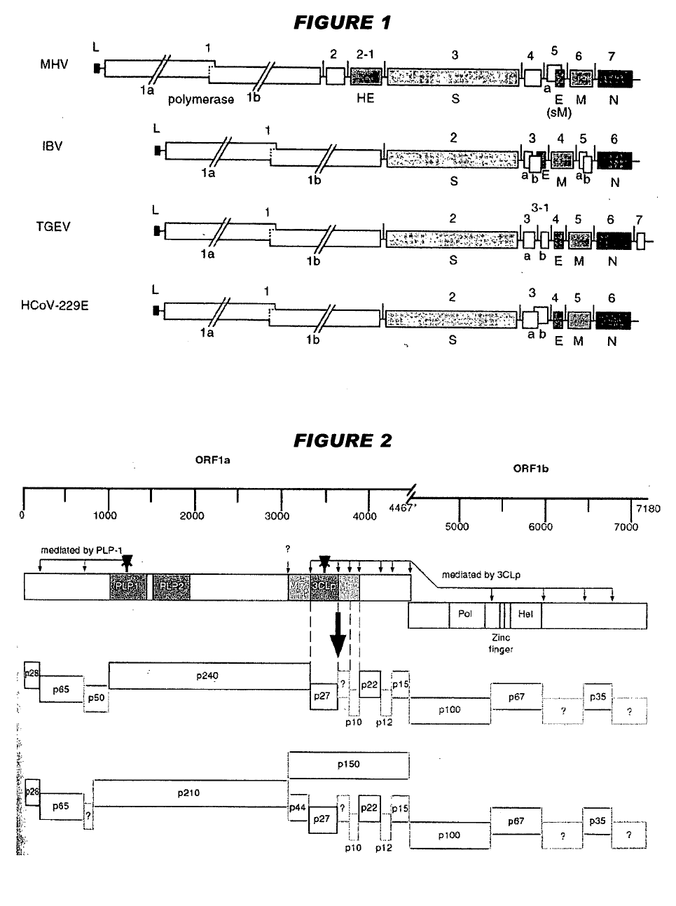

One skilled in the art would be able to identify and clone additional genomic regions using a variety of standard cloning techniques, such as, for example, using random primer RT-PCR and detection of sequences overlapping one or more of the above sequences, and/or using oligonucleotide primers, e.g., degenerate primers, based on the sequences provided herein (see FIGS. 1-5, FIGS. 8-11, SEQ ID NOS: 3-20).

Cloning, sequencing and identification of SARS virus by one skilled in the art can be further facilitated by the use of polynucleotide sequences, particularly RNA polymerase sequences, from related Coronaviruses.

Sequence homology of new viral isolates with the known SARS isolates described above can be readily determined by one skilled in the art. New SARS isolates may be identified by a percent homology of viral nucleotide sequences of 99%, 95%, 92%, 90%, 85%, or 80% homology of the new virus to known SARS viral polynucleotide sequences. New SARS isolates may also be identified by percent homology of 99%, 95%, 92%, 90%, 85%, or 80% homology of the polypeptides encoded by the polynucleotides of the new virus and the polypeptides encoded by known SARS virus.

New SARS isolates may also be identified by a percent homology of 99%, 95%, 92%, 90%, 85%, or 80% homology of the polynucleotide sequence for specific genomic regions for the new virus with the polynucleotide sequence for specific genomic regions of the known SARS viruses. Additionally, new SARS isolates may be identified by a percent homology of 99%, 95%, 92%, 90%, 85%, or 80% homology of the polypeptide sequence encoded by the polynucleotide of specific genomic regions of the new SARS virus to the polypeptide sequence encoded by the polynucleotides of specific regions of the known SARS virus. These genomic regions may include regions (e.g., gene products) which are typically in common among numerous coronaviruses, as well as group specific regions (e.g., antigenic groups), such as, for example, any one of the following genomic regions which could be readily identified by a virologist skilled in the art: 5′untranslated region (UTR), leader sequence, ORF1a, ORF1b, nonstructural protein 2 (NS2), hemagglutinin-esterase glycoprotein (HE) (also referred to as E3), spike glycoprotein (S) (also referred to as E2), ORF3a, ORF3b, ORF3x, nonstructural protein 4 (NS4), envelope (small membrane) protein (E) (also referred to as sM), membrane glycoprotein (M) (also referred to as E1), ORF5a, ORF5b, nucleocapsid phosphoprotein (N), ORF7a, ORF7b, intergenic sequences, 3′UTR, or RNA dependent RNA polymerase (pol). The SARS virus may have identifiable genomic regions with one or more the above-identified genomic regions. A SARS viral antigen includes a protein encoded by any one of these genomic regions. A SARS viral antigen may be a protein or a fragment thereof, which is highly conserved with coronaviruses. A SARS viral antigen may be a protein or fragment thereof, which is specific to the SARS virus (as compared to known cornaviruses). (See, FIGS. 1-5, FIGS. 8-11, SEQ ID NOS: 3-20).

One skilled in the art could also recognize electron microscopy of a SARS virus infected mammalian cell. Electron microscopy of SARS infected cells are shown in the Lancet paper. As discussed in the paper, electron microscopy of negative stained (3% potassium phospho-tungstate, pH 7.0) ultracentrifuged cell-culture extracts of SARS infected fetal rhesus kidney cells show the presence of pleomorphic enveloped virus particles of around 80-90 nm (range 70-130 nm) in diameter with surface morphology compatible with a coronavirus (see Lancet paper, FIG. 1). Thin-section electron microscopy of infected cells reveals virus particles of 55-90 nm diameter within smooth walled vesicles in the cytoplasm (see Lancet paper, FIG. 2B). Electron microscopy can also be used to observe virus particles at the cell surface. Electron microscopy of a human lung biopsy sample depicts similar viral morphology. See Lancet paper FIG. 2A.

I. SARS Polypeptides and Polynucleotides

The invention relates to nucleic acids and proteins from SARS virus. Such polynucleotides and polypeptides are exemplified further below.

In one embodiment, the polynucleotides of the invention do not include one of the following five primers, disclosed at http://content.nejm.org/cgi/reprint/NEJMoa030781v2.pdf: SEQ ID NOs: 6034-38.

The invention also includes polynucleotide sequences which can be used as probes for diagnostic reagents, kits (comprising such reagents) and methods which can be used to diagnose or identify the presence or absence of a SARS virus in a biological sample. The invention includes a polynucleotide sequence comprising one or more of the primer sequences identified in SEQ ID NOS: 21-1020. The invention further includes polynucleotide sequence comprising the complement of one or more of the primer sequences identified in SEQ ID NOS: 21-1020.

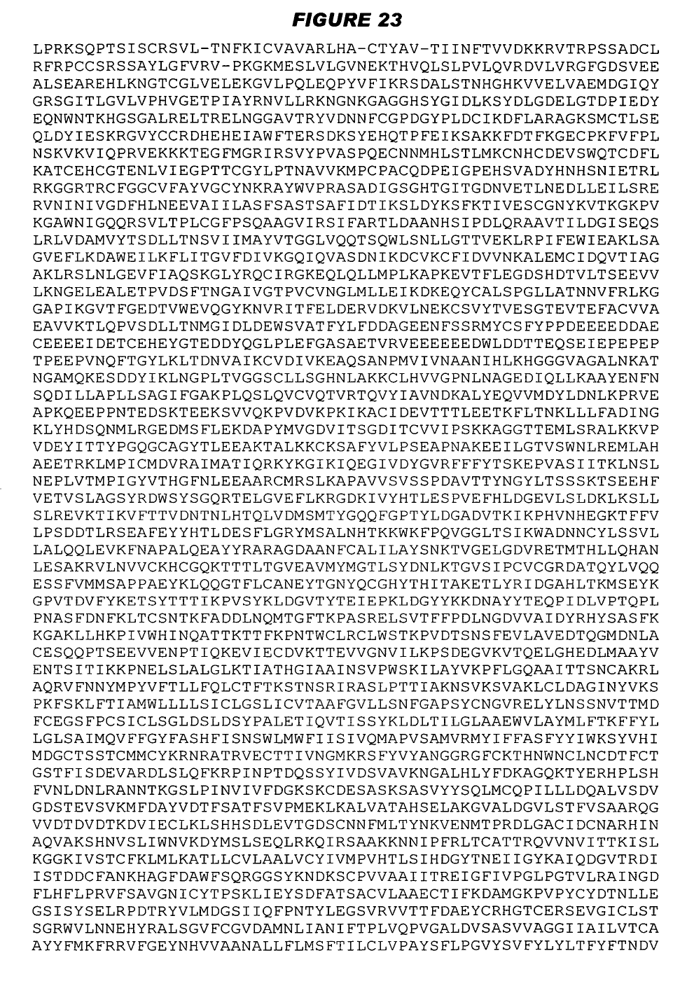

The invention includes a polypeptide sequence comprising an amino acid sequence from the sequence shown in FIG. 23. Such amino acid sequences are SEQ ID NOS: 6588-6809. The invention includes polypeptides comprising an amino acid sequence having sequence identity to these sequences, and the invention includes a fragment of a polypeptide comprising one of these sequences.

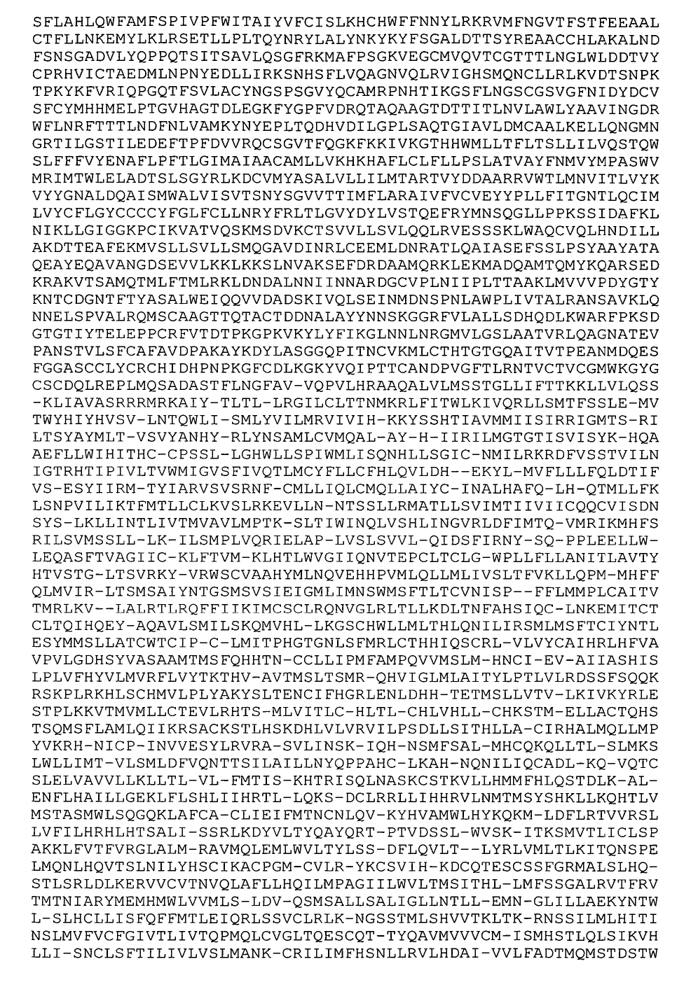

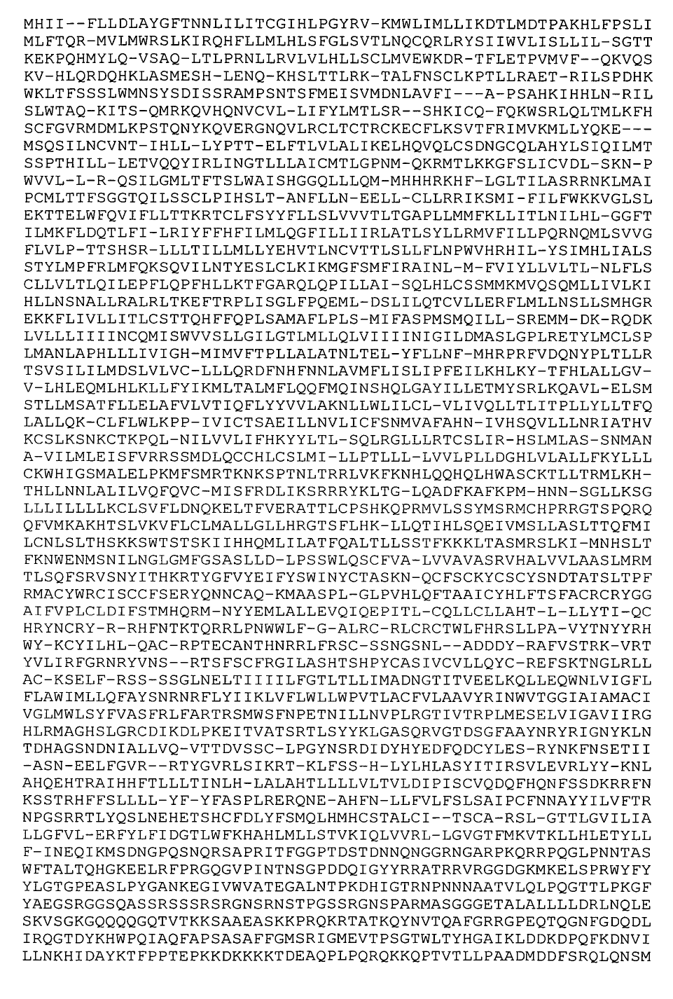

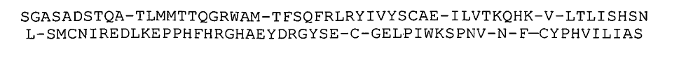

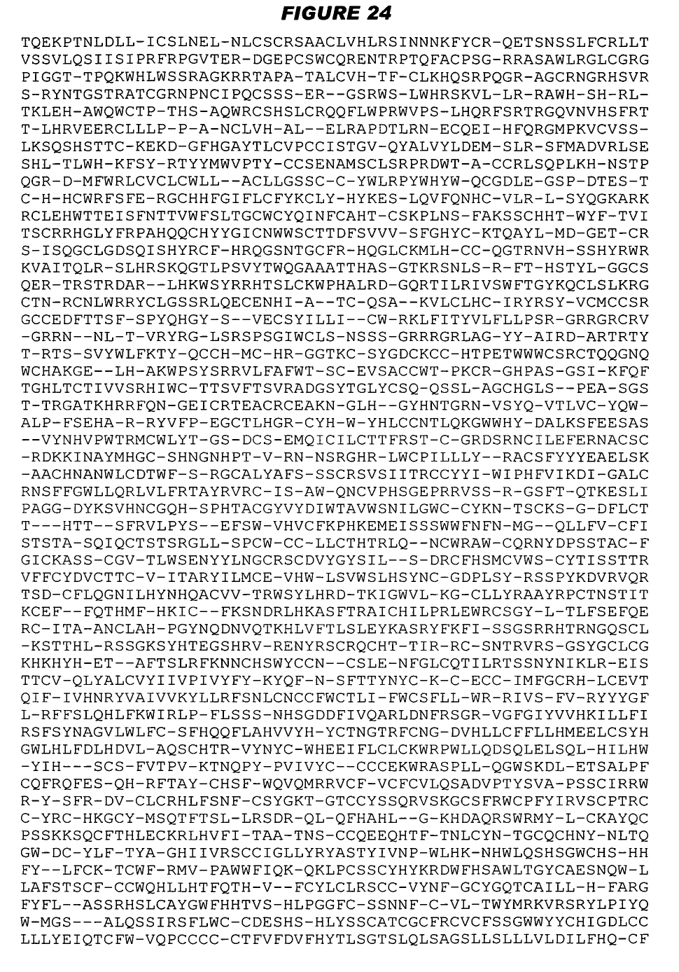

The invention includes a polypeptide comprising an amino acid sequence from the sequence shown in FIG. 24. Such amino acid sequences are SEQ ID NOS: 6810-7179. The invention includes a protein comprising an amino acid sequence having sequence identity to these sequences, and the invention includes a fragment of a protein comprising one of these sequences.

The invention includes a protein comprising SEQ ID NO: 6039. The invention includes a polypeptide comprising an amino acid sequence having sequence identity to SEQ ID NO: 6039. The invention includes a fragment of a polypeptide comprising SEQ ID NO: 6039. The invention includes a diagnostic kit comprising a polypeptide comprising SEQ ID NO: 6039, or a fragment thereof. The invention includes a diagnostic kit comprising a polynucleotide sequence encoding SEQ ID NO: 6039, or a fragment thereof. The invention includes an immunogenic composition comprising a polypeptide comprising SEQ ID NO: 6039, or a fragment thereof. The invention includes an antibody which recognizes a polypeptide comprising SEQ ID NO: 6039, or a fragment thereof. SEQ ID NO: 6039 demonstrates functional homology with ORF1a of coronaviruses.

Predicted transmembrane or hydrophobic regions of SEQ ID NO: 6039 are identified below. Although the polyprotein of coronaviruses is proteolytically cleaved into numerous smaller proteins, hydrophobic domains in the polyprotein are known to mediate the membrane association of the replication complex and to be able to dramatically alter the architecture of host cell membranes. Accordingly, the hydrophobic domains of the polyprotein are targets for genetic mutation to develop attenuated SARS virus vaccines. The hydrophobic domains are also targets for small molecule inhibitors of the SARS virus. The hydrophobic domains may also be used to generate antibodies specific to those regions to treat or prevent SARS virus infection.

Predicted Transmembrane Helices in SEQ ID NO: 6039

The sequence positions in brackets denominate the core region.

Only scores above 500 are considered significant.

| | | | |
|---|---|---|---|
| from | to | score | center |

Inside to outside helices: 43 found

| | | | |
|---|---|---|---|
| 100 (100) | 118 (116) | 103 | 107 |
| 473 (473) | 488 (488) | 1003 | 481 |
| 529 (532) | 549 (549) | 541 | 539 |
| 584 (584) | 606 (601) | 1049 | 594 |
| 773 (773) | 791 (789) | 514 | 782 |
| 1071 (1071) | 1089 (1086) | 243 | 1078 |
| 1121 (1121) | 1137 (1137) | 459 | 1130 |
| 1679 (1679) | 1696 (1696) | 404 | 1686 |
| 2098 (2102) | 2119 (2116) | 509 | 2109 |
| 2145 (2145) | 2160 (2160) | 797 | 2153 |
| 2206 (2209) | 2224 (2224) | 2686 | 2216 |
| 2316 (2316) | 2332 (2332) | 2123 | 2325 |
| 2335 (2339) | 2358 (2354) | 2101 | 2346 |
| 2373 (2373) | 2390 (2390) | 532 | 2380 |
| 2597 (2600) | 2615 (2615) | 307 | 2607 |
| 2753 (2753) | 2770 (2768) | 2242 | 2760 |
| 2831 (2833) | 2854 (2851) | 759 | 2841 |
| 2879 (2882) | 2900 (2897) | 526 | 2889 |
| 2990 (2996) | 3012 (3010) | 1289 | 3003 |
| 3024 (3024) | 3042 (3039) | 2281 | 3032 |
| 3054 (3058) | 3075 (3072) | 2536 | 3065 |
| 3105 (3109) | 3127 (3123) | 2010 | 3116 |
| 3143 (3143) | 3163 (3159) | 184 | 3152 |
| 3253 (3255) | 3272 (3272) | 319 | 3262 |
| 3346 (3346) | 3366 (3366) | 203 | 3356 |
| 3375 (3375) | 3392 (3392) | 305 | 3384 |
| 3438 (3438) | 3455 (3453) | 1021 | 3445 |
| 3559 (3567) | 3584 (3581) | 1885 | 3574 |
| 3589 (3589) | 3606 (3604) | 2018 | 3596 |
| 3611 (3611) | 3629 (3629) | 2304 | 3621 |
| 3659 (3659) | 3674 (3674) | 1561 | 3667 |
| 3756 (3758) | 3777 (3774) | 2352 | 3767 |
| 3890 (3890) | 3904 (3904) | 485 | 3897 |
| 3916 (3919) | 3934 (3934) | 241 | 3926 |
| 4035 (4035) | 4051 (4051) | 335 | 4044 |
| 4217 (4217) | 4232 (4232) | 272 | 4224 |
| 4239 (4239) | 4257 (4254) | 402 | 4247 |

Outside to inside helices: 43 found

| | | | |
|---|---|---|---|
| 94 (97) | 118 (112) | 291 | 104 |
| 400 (400) | 418 (415) | 243 | 407 |
| 473 (473) | 488 (488) | 1113 | 481 |
| 523 (528) | 548 (548) | 285 | 538 |
| 583 (583) | 606 (601) | 662 | 593 |
| 776 (776) | 791 (791) | 1435 | 783 |
| 1068 (1071) | 1089 (1086) | 370 | 1078 |
| 1121 (1121) | 1137 (1137) | 455 | 1130 |
| 1679 (1679) | 1696 (1694) | 340 | 1686 |
| 2098 (2098) | 2119 (2116) | 678 | 2109 |
| 2148 (2148) | 2163 (2163) | 434 | 2155 |
| 2208 (2210) | 2231 (2226) | 2389 | 2219 |
| 2309 (2309) | 2332 (2326) | 1773 | 2318 |
| 2342 (2342) | 2368 (2360) | 1666 | 2353 |
| 2373 (2373) | 2390 (2390) | 254 | 2380 |
| 2753 (2755) | 2770 (2770) | 2119 | 2763 |
| 2832 (2835) | 2854 (2851) | 687 | 2844 |
| 2858 (2858) | 2873 (2873) | 253 | 2866 |
| 2879 (2882) | 2899 (2899) | 400 | 2889 |
| 2990 (2990) | 3005 (3005) | 875 | 2998 |
| 3020 (3024) | 3042 (3042) | 2795 | 3032 |
| 3059 (3059) | 3075 (3075) | 2137 | 3067 |
| 3105 (3108) | 3127 (3123) | 1902 | 3115 |
| 3142 (3145) | 3162 (3162) | 540 | 3152 |
| 3343 (3351) | 3366 (3366) | 496 | 3358 |
| 3437 (3437) | 3453 (3453) | 848 | 3444 |
| 3489 (3491) | 3508 (3505) | 302 | 3498 |
| 3560 (3560) | 3577 (3577) | 1460 | 3569 |
| 3591 (3591) | 3606 (3606) | 2193 | 3598 |
| 3610 (3610) | 3627 (3627) | 1484 | 3620 |
| 3656 (3658) | 3678 (3675) | 1240 | 3668 |
| 3681 (3684) | 3701 (3699) | 590 | 3691 |
| 3710 (3713) | 3738 (3728) | 1696 | 3721 |
| 3723 (3723) | 3738 (3738) | 1670 | 3730 |
| 3760 (3760) | 3777 (3775) | 2367 | 3767 |
| 3881 (3884) | 3902 (3900) | 249 | 3892 |
| 4099 (4099) | 4114 (4114) | 389 | 4106 |
| 4234 (4234) | 4254 (4249) | 325 | 4241 |
| 4338 (4341) | 4360 (4360) | 505 | 4348 |

Accordingly, the invention includes a polypeptide comprising a fragment of SEQ ID NO: 6039, wherein said fragment comprises an amino acid sequence including one or more of the hydrophobic transmembrane sequences identified above. The invention includes a polypeptide comprising a fragment of SEQ ID NO: 6039 wherein said fragment comprises one or more of the following polypeptide sequences of SEQ ID NO: 6039: 473-488; 529-549, 584-606, 773-791, 2098-2119, 2145-2160, 2206-2224, 2316-2332, 2335-2358, 2373-2390, 2753-2770, 2831-2854, 2879-2990, 2990-3012, 3024-3042, 3054-3075, 3105-3127, 3438-3455, 3559-3584, 3589-3606, 3611-3629, 3659-3674, 3756-3777, 473-488, 583-606, 776-791, 2098-2119, 2208-2231, 2309-2332, 2342-2368, 2753-2770, 2832-2854, 2990-3005, 3020-3042, 3059-3075, 3105-3127, 3142-3162, 3437-3453, 3560-3577, 3591-3606, 3610-3627, 3656-3678, 3710-3738, 3723-3738, and 3760-3777. Preferably, the fragment comprises one or more of the following polypeptide sequences of SEQ ID NO: 6039: 2206-2224, 2316-2332, 2335-2358, 2753-2770, 3024-3042, 3054-3075, 3105-3127, 3589-3606, 3611-3629, 3756-3777, 2208-2231, 2753-2770, 3020-3042, 3059-3075, and 3591-3606. Preferably, the fragment comprises one or more of the following polypeptide sequences of SEQ ID NO: 6039: 2206-2224 and 3020-3042. The invention also includes polynucleotides encoding each of the polypeptide fragments identified above.

The invention includes an attenuated SARS virus wherein said attenuated SARS virus contains an addition, deletion or substitution in the polynucleotides encoding for one of the hydrophobic domains identified above. The invention also includes a method for creating an attenuated SARS virus comprising mutating a SARS virus by adding, deleting or substituting the viral genome of the SARS virus to alter the coding of one or more of the hydrophobic domains of SEQ ID NO: 6039 identified above.

The invention includes an antibody which specifically identifies one or more of the hydrophobic regions of SEQ ID NO: 6039 identified above. The invention includes a small molecule which binds to, interferes with the hydrophobicity of or otherwise disrupts one or more of the hydrophobic regions of SEQ ID NO: 6039 identified above.

Predicted N-glycosylation sites of SEQ ID NO: 6039 are identified in the chart below.

Prediction of N-glycosylation Sites in SEQ ID NO: 6039

| | | | | Jury | NGlyc | |
|---|---|---|---|---|---|---|
| Position | | | Potential | agreement | result | |
| 48 | NGTC | SEQ ID NO: 7180 | 0.6371 | (7/9) | + | |
| 389 | NHSN | SEQ ID NO: 7181 | 0.6132 | (6/9) | + | |
| 916 | NFSS | SEQ ID NO: 7182 | 0.5807 | (7/9) | + | |
| 1628 | NHTK | SEQ ID NO: 7183 | 0.5610 | (7/9) | + | |
| 1696 | NKTV | SEQ ID NO: 7184 | 0.5297 | (5/9) | + | |
| 2031 | NPTI | SEQ ID NO: 9764 | 0.5299 | (5/9) | + | WARNING: PRO- |
| | | | | | | X1. |
| 2249 | NSSN | SEQ ID NO: 7185 | 0.6329 | (9/9) | ++ | |
| 2459 | NPTD | SEQ ID NO: 9765 | 0.5599 | (6/9) | + | WARNING: PRO- |
| | | | | | | X1. |
| 2685 | NVSL | SEQ ID NO: 7186 | 0.6071 | (8/9) | + | |
| 4233 | NATE | SEQ ID NO: 7187 | 0.6144 | (7/9) | + | |

Accordingly, the invention comprises a fragment of SEQ ID NO: 6039 wherein said fragment comprises an amino acid sequence which includes one or more of the N-glycosylation sites identified above. Preferably, the fragment comprises one or more sequences selected from the group consisting of SEQ ID NOS: 7180-7187 & 9764-9765. Preferably, the fragment comprises the amino acid sequence NSSN (SEQ ID NO: 7185).

The invention includes a polypeptide comprising a fragment of SEQ ID NO: 6039 wherein said fragment does not include one or more of the glycosylation sites identified above. The invention also includes a polynucleotide encoding such a polypeptide.

T-epitopes for SEQ ID NO: 6039 are identified in Table 13. The invention includes a polypeptide for use as an antigen, wherein the polypeptide comprises: (a) an amino acid sequence selected from the group consisting of the T-epitope sequences identified as SEQ ID NOS: 7400-7639; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified as SEQ ID NOS: 7400-7639, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus.

The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The ORF1a and ORF1b sequences of coronaviruses are typically translated as a single ORF1ab polyprotein. Slippage of the ribosome during translation generates an a-1 frameshift. One region of such slippage is illustrated below:

```
      gggttttacacttagaaacacagtctgtaccgtctgcggaatgtggaaaggttatggctgtagttgtga
+1 G F T L R N T V C T V C G M W K G Y G C S C D
+3 G F Y T - K H S L Y R L R N V E R L W L - L -
      ccaactccgcgaacccttgatgcagtctgcggatgcatcaacgtttttaaacgggtttgcggtgtaagt
+1 Q L R E P L M Q S A D A S T F L N G F A V - V
+3 P T P R T L D A V C G C I N V F K R V C G V S
      gcagcccgtcttacaccgtgcggcacaggcactagtactg (SEQ ID NO: 7224)
+1 Q P V L H R A A Q A L V L (SEQ ID NOS: 7225-7226)
+3 A A R L T P C G T G T S T (SEQ ID NOS: 7227-7229)
```

which would generate the following translational slippage (SEQ ID NOS: 7230-7231):

ccaactccgcgaacccttgatgcagtctgcggatgcatcaacgtttttaaacgggtttgcggtgtaagt
Q L R E P L M Q S A D A S T F L N R V C G V S

Accordingly, the invention includes a polypeptide comprising SEQ ID NO: 7232. The invention includes a polypeptide comprising an amino acid sequence having sequence identity to SEQ ID NO: 7232. The invention includes a fragment of a polypeptide comprising SEQ ID NO: 7232 The invention includes a diagnostic kit comprising a polypeptide comprising SEQ ID NO: 7232 or a fragment thereof. The invention includes a diagnostic kit comprising a polynucleotide sequence encoding SEQ ID NO: 7232 or a fragment thereof. The invention includes an immunogenic composition comprising a polypeptide comprising SEQ ID NO: 7232 or a fragment thereof. The invention includes an antibody which recognizes a polypeptide comprising SEQ ID NO: 7232 or a fragment thereof.

The invention also includes a polypeptide comprising amino acid sequence X₁—X₂—X₃, where X₁ is SEQ ID NO: 7233, X₂ is from one to ten amino acids, and X₃ is SEQ ID NO: 7234. X₂ can comprise any sequence of one to ten amino acids (SEQ ID NOS: 7235-7244) but, in preferred embodiments, X₂ is selected from the group consisting of F, FL, FLN, FLNR (SEQ ID NO: 7245), FLNRV (SEQ ID NO: 7246) and FLNRVC (SEQ ID NO: 7247). Preferably, X₂ is SEQ ID NO: 7247. These preferred embodiments are shown as SEQ ID NOS: 7248-7253.

The invention includes a polypeptide comprising an amino acid sequence having sequence identity to said amino acid sequences X₁—X₂—X₃. The invention includes a fragment of a polypeptide comprising said amino acid sequences X₁—X₂—X₃. The invention includes a diagnostic kit comprising a polypeptide comprising said amino acid sequences X₁—X₂—X₃ or a fragment thereof. The invention includes a diagnostic kit comprising a polynucleotide sequence encoding said amino acid sequences X₁—X₂—X₃ or a fragment thereof. The invention includes an immunogenic composition comprising a polypeptide comprising said amino acid sequences X₁—X₂—X₃ or a fragment thereof. The invention includes an antibody which recognizes a polypeptide comprising said amino acid sequences X₁—X₂—X₃ or a fragment thereof.

The amino acid sequences X₁—X₂—X₃ (i.e. SEQ ID NOS: 7235-7244) demonstrate functional homology with the polyprotein of murine hepatitis virus. This polyprotein is cleaved to produce multiple proteins. Proteins which can be generated from the X₁—X₂—X₃ polyprotein, where X₂ is six amino acids (SEQ ID NO: 7240) are listed below.

| | | Coordinates in |
|---|---|---|
| Mouse virus protein | Coordinates in Mouse virus | SEQ ID NO: 7240 |
| Nsp2 | 3334-3636 | 3241-3546 |
| Nsp3 | 3637-3923 | 3547-3836 |
| Nsp4 | 3924-4015 (or 4012) | 3837-3919 |
| Nsp5 | 4016 (or 4013)-4209 | 3920-4117 |
| Nsp6 | 4210-4319 | 4118-4230 |
| Nsp7 | 4320-4456 | 4231-4369 |
| Nsp9 | 4457-5384 | 4370-5301 |
| Nsp10 | 5385-5984 | 5302-5902 |
| Nsp11 | 5985-6505 | 5903-6429 |
| Nsp12 | 6506-6879 | 6430-6775 |
| Nsp13 | 6880-7178 | 6776-7073 |

The invention includes a fragment of the amino acid sequence X₁—X₂—X₃ (i.e. SEQ ID NOS: 7235-7244) wherein the fragment comprises one of the polypeptide sequences identified in the above table. The invention further includes a fragment of the amino acid sequence X₁—X₂—X₃ wherein said fragment comprises a polypeptide sequence which has a serine at its N-terminus and a glutamine at its C-terminus. The invention further includes a fragment of the amino acid sequence X₁—X₂—X₃ wherein said fragment comprises a polypeptide sequence which has an Alanine at its N-terminus and a glutamine at its C-terminus. The invention further includes a fragment of the amino acid sequence X₁—X₂—X₃ wherein said fragment comprises a polypeptide sequence which has a Asparagine at its N-terminus and a glutamine at its C-terminus. The invention further includes a fragment of the amino acid sequence X₁—X₂—X₃ wherein said fragment comprises a Cysteine at its N-terminus and a Glutamine at its C-terminus. Each of the fragments identified above can be used in fusion proteins.

The invention includes a diagnostic kit comprising a polypeptide comprising at least one of the fragments of the amino acid sequence X₁—X₂—X₃ (i.e. SEQ ID NOS: 7235-7244) identified in the above paragraph. The invention includes a diagnostic kit comprising a polynucleotide sequence encoding at least one of the fragments of the amino acid sequence X₁—X₂—X₃ identified in the above paragraph. The invention includes an immunogenic composition comprising a polypeptide comprising at least one of the fragments of the amino acid sequence X₁—X₂—X₃ identified in the above paragraph. The invention includes an antibody which recognizes a polypeptide comprising at least one of the fragments of the amino acid sequence X₁—X₂—X₃ identified in the above paragraph.

Predicted N-glycosylation sites of the amino acid sequence X₁—X₂—X₃ when X₂ is six amino acids are identified at the asparagines located at the following amino acid positions 48; 389; 556; 916; 1628; 1696; 1899; 2079; 2249; 2252; 2507; 2685; 3303; 3373; 3382; 3720; 4150; 4233; 4240; 5016; 5280; 5403; 5558; 5650; 5905; 6031; 6130; 6474; 6918; 6973. Accordingly, the invention comprises a fragment of SEQ ID NO: 7239 wherein said fragment is at least ten amino acids and wherein said fragment comprises one or more of the asparagines from the amino acid positions of SEQ ID NO: 7239 selected from the group consisting of 8; 389; 556; 916; 1628; 1696; 1899; 2079; 2249; 2252; 2507; 2685; 3303; 3373; 3382; 3720; 4150; 4233; 4240; 5016; 5280; 5403; 5558; 5650; 5905; 6031; 6130; 6474; 6918; and 6973.

A zinc binding region 2 site within SEQ ID NOS: 7235-7244 is identified at amino acid residues 2102-2112 (SEQ ID NO: 7254 HGIAAINSVPW). The polypeptide of SEQ ID NOS: 7235-7244 will be processed by the SARS virus into multiple peptides. This zinc binding region falls within the nsp1 region of the polypeptide. SEQ ID NO: 7254 is a target for screening of chemical inhibitors to the SARS virus. The invention includes a polypeptide comprises SEQ ID NO: 7254. The invention includes a polynucleotide encoding the polypeptide sequence of SEQ ID NO: 7254. The invention includes a method of screening SEQ ID NO: 7254 for an inhibitor. The invention includes the recombinant expression of SEQ ID NO: 7254 in a host cell. The invention includes a fragment of SEQ ID NOS: 7235-7244, wherein said fragment comprises SEQ ID NO: 7254. The invention includes a polypeptide comprising SEQ ID NO: 7254 wherein said polypeptide is complexed with a zinc ion. The invention includes a small molecule which prevents a zinc ion from complexing with the polypeptide of SEQ ID NO: 7254. The invention includes a fusion protein wherein said fusion protein comprises SEQ ID NO: 7254.

The polyprotein encoded by the SARS virus will contain at least two protease domains: a papain-like cystein protease (PLP) and a chymotrypsin-picornavirus 3C-like protease (3CLp). (There may be more than one copy of the PLP domain). These proteases function to cleave the polyprotein into multiple smaller proteins. The 3C-like protease, also known as the “main protease” or Mpro, is itself cleaved from the polyprotein by its own autoprotease activity. See generally, Chapter 35 of Fields Virology (2nd ed), Fields et al. (eds.), B.N. Raven Press, New York, N.Y., and Anand et al., EMBO Journal (2002) 21 (13): 3213-3224. This 3CLp generally corresponds with the Nsp2 region identified above.

The SARS virus 3CLp protein is further characterized by SEQ ID NO: 6569 (also SEQ ID NO: 9769), as shown in FIG. 15.

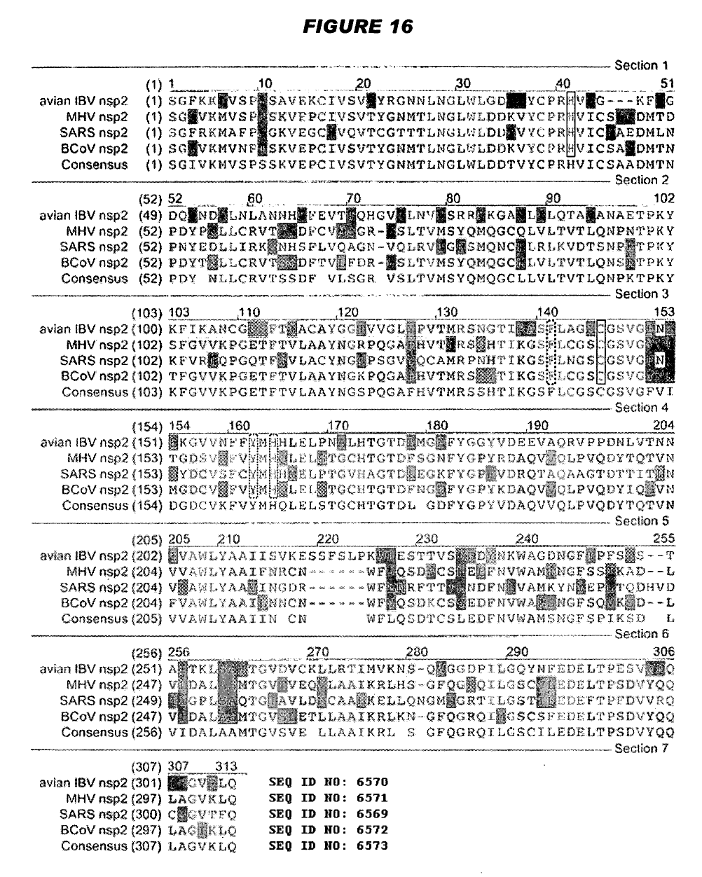

FIG. 16 also illustrates the SARS virus 3CLp, in allignment with the 3CLp of avian infectious bronchitis (IBV; SEQ ID NO: 6570), mouse hepatitis virus (MHV; SEQ ID NO: 6571), and bovine coronavirus (BCoV; SEQ ID NO: 6572). Accordingly, the invention includes a polypeptide sequence comprising SEQ ID NO: 6569, or a fragment thereof, or a polypeptide sequence having sequence identity thereto. The invention further includes a polynucleotide sequence encoding SEQ ID NO: 6569, or a fragment thereof. The invention includes a polynucleotide sequence encoding a polypeptide sequence having sequence identity to SEQ ID NO: 6569.

The invention further includes a method of screening for an inhibitor of the SARS virus 3CLp protein. In one embodiment, the invention includes a method of screening for an inhibitor of SEQ ID NO: 6569. The invention includes a method of recombinantly expressing the SARS virus 3CLp protein in a host cell. The invention includes a method of recombinantly expressing a polypeptide sequence comprising SEQ ID NO: 6569 or an enzymatically active fragment thereof or a polypeptide sequence having sequence identity thereto. The invention includes a small molecule which inhibits or reduces the proteolytic activity of the SARS virus 3CLp protein. The invention includes a small molecule which inhibits or reduced the proteolytic activity of the polypeptide comprising SEQ ID NO: 6569.

Catalytic residues of the SARS virus 3CLp are identified in FIG. 15 and 16. Specifically, a catalytic histidine and a catalytic cysteine are identified. Such catalytic sites are targets for small molecules which could inhibit or reduce the protease activity of 3CLp. Accordingly, the invention includes a polypeptide comprising a fragment of SEQ ID NO: 6569, wherein said fragment comprises at least one catalytic site. Preferably, the catalytic site is selected from the group consisting of the indicated catalytic histidine and the catalytic cysteine in FIG. 15 and 16. The invention includes a polynucleotide encoding a polypeptide, wherein said polypeptide comprises a fragment of SEQ ID NO: 6569, wherein said fragment comprises at least one catalytic site. Preferably, the catalytic site is selected from the group consisting of the indicated catalytic histidine and the catalytic cysteine.

The invention further includes a method of screening a compound library to identify a small molecule which inhbits a catalytic site of a SARS virus 3CLp. Preferably, the 3CLp comprises SEQ ID NO: 6569, or a fragment thereof, or a sequence having sequence identity thereto. The catalytic site is preferably selected from the group consisting of the indicated catalytic histidine and the catalytic cysteine in FIG. 15 and 16.

The invention includes a small molecule which inhibits the catalytic site of a SARS virus 3CLp. Preferably, the 3CLp comprises SEQ ID NO: 6569, or a fragment thereof, or a sequence having sequence identity thereto. The catalytic site is preferably selected from the group consisting of the indicated catalytic histidine and the catalytic cysteine in FIG. 15 and 16.

Residues of the substrate site of the SARS virus 3CLp are identified in FIG. 15 and 16. Specifically, a substrate site is indicated at a phenylalanine, a tyrosine and a histidine. Such substrate sites are targets for small molecules which could inhibit or reduce the protease activity of 3CLp. Accordingly, the invention includes a polypeptide comprising a fragment of SEQ ID NO: 6569, wherein said fragment comprises at least one substrate site. Preferably, the substrate site is selected from the group consisting of the indicated substrate phenylalanine, tyrosine and histidine in FIG. 15 and 16. The invention includes a polynucleotide encoding a polypeptide, wherein said polypeptide comprises a fragment of SEQ ID NO: 6569, wherein said fragment comprises at least one substrate site. Preferably, the substrate site is selected from the group consisting of the indicated substrate phenylalanine, tyrosine and histidine in FIG. 15 and 16.

The invention further includes a method of screening a compound library to identify a small molecule which blocks a substrate site of a SARS virus 3CLp. Preferably, the 3CLp comprises SEQ ID NO: 6569, or a fragment thereof, or a sequence having sequence identity thereto. The substrate site is preferably selected from the group consisting of the indicated substrate phenylalanine, tyrosine and histidine in FIG. 15 and 16.

The invention includes a small molecule which inhibits the substrate site of a SARS virus 3CLp. Preferably, the 3CLp comprises SEQ ID NO: 6569, or a fragment thereof, or a sequence having sequence identity thereto. The substrate site is preferably selected from the group consisting of the indicated substrate phenylalanine, tyrosine and histidine in FIG. 15 and 16.

The invention further includes a diagnostic kit comprising a polynucleotide encoding a SARS virus 3CLp or a fragment thereof. Preferably, the SARS virus 3CLp comprising SEQ ID NO: 6569 or a fragment thereof or a polypeptide sequence having sequence identity thereto. Preferably, the fragment comprising one or more sites selected from the group consisting of a catalytic site and a substrate site. Preferably, the catalytic site is selected from the group consisting of one or more of the sites identified in FIG. 15 and 16. Preferably, the substrate site is selected from the group consisting of one or more of the sites identified in FIG. 15 and 16.

The invention further comprises a diagnostic kit comprising an antibody specific to a SARS virus 3CLp or a fragment thereof. Preferably, the antibody is specific to the polypeptide comprising SEQ ID NO: 6569 or a fragment thereof or a polypeptide sequence having sequence identity thereto. Preferably, the antibody is specific to one or more sites of a SARS virus 3CLp selected from the group consisting of a catalytic site and a substrate site. Preferably, the catalytic site is selected from the group consisting of one or more of the sites identified in FIG. 15 and 16. Preferably, the substrate site is selected from the group consisting of one or more of the sites identified in FIG. 15 and 16.

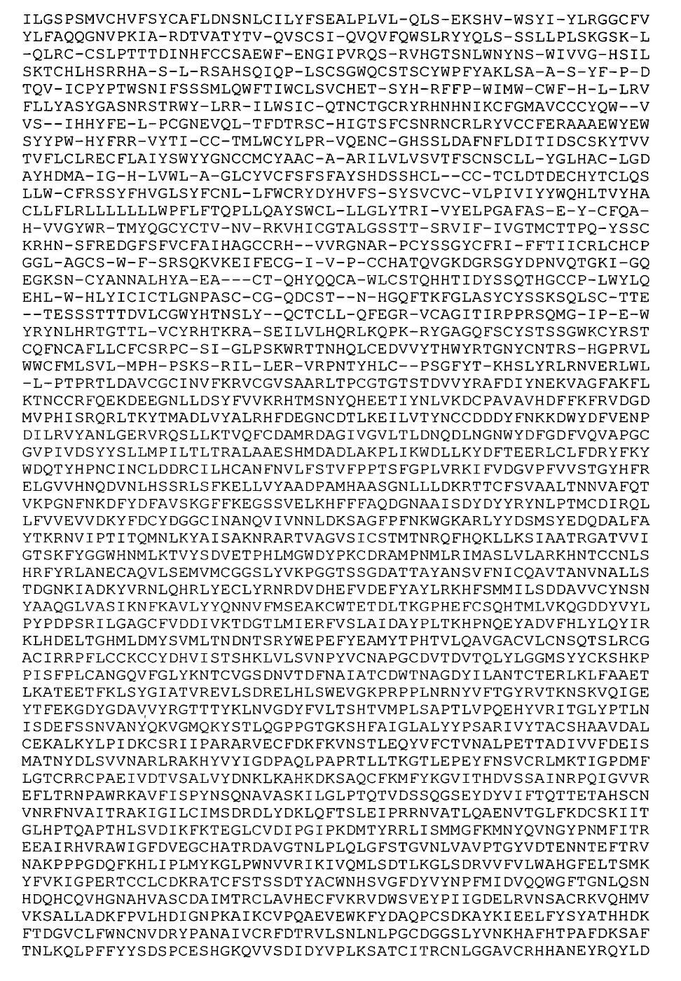

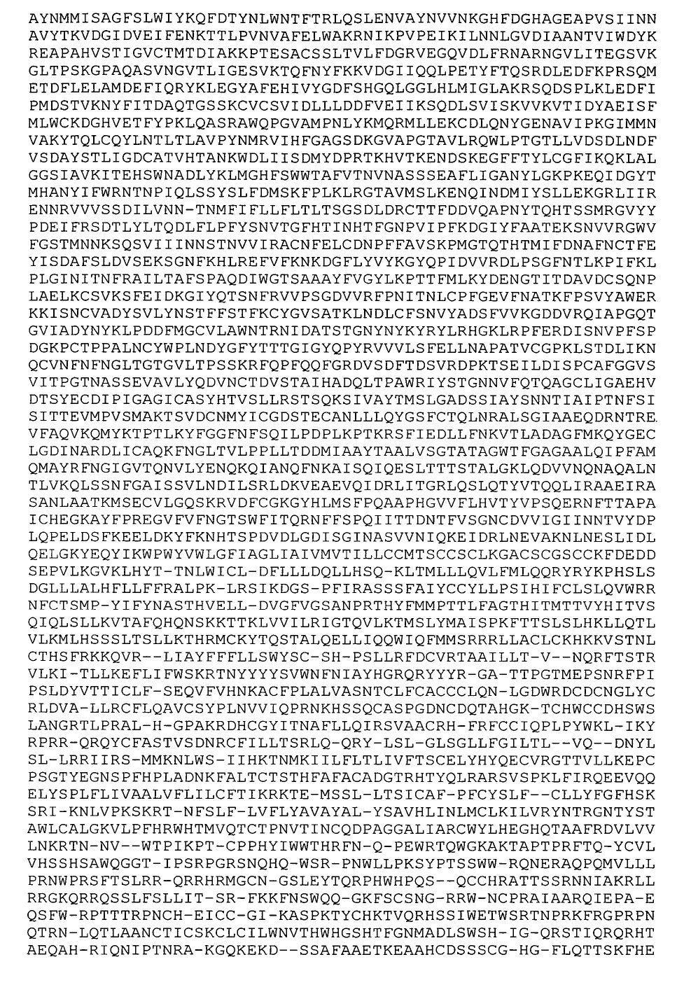

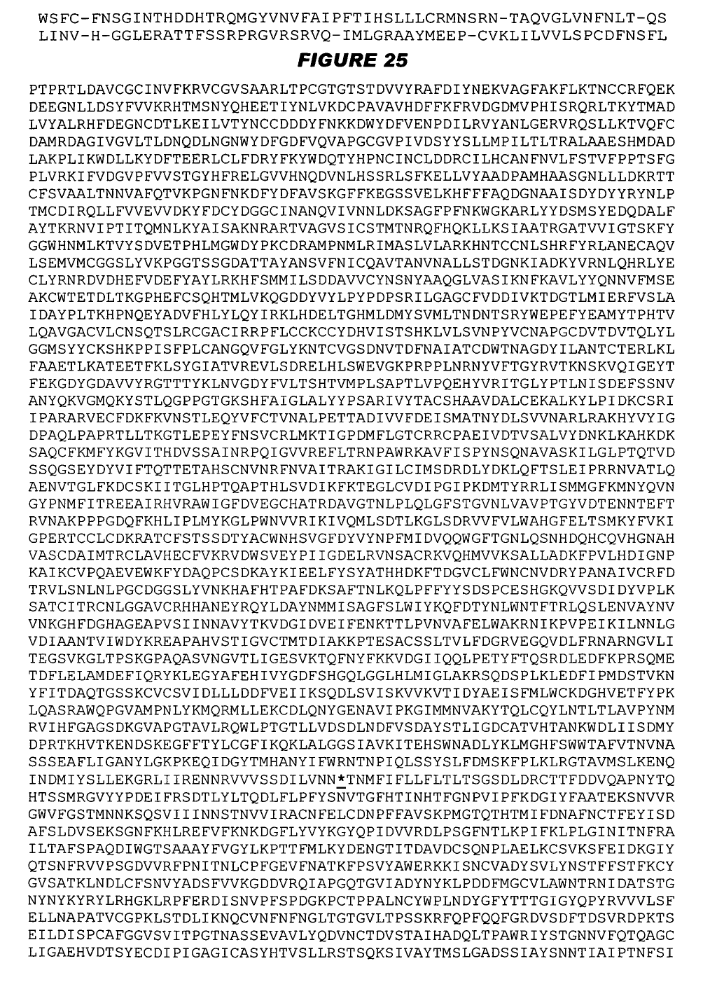

The invention includes a polypeptide comprising an amino acid sequence from the sequence shown in FIG. 25. The two amino acid sequences within FIG. 25, separated by a *, are SEQ ID NOS: 7188 & 7189. The invention includes a polypeptide comprising an amino acid sequence having sequence identity to the FIG. 25 translation. The invention includes a fragment of a polypeptide comprising the FIG. 25 sequence. The invention includes a diagnostic kit comprising a polypeptide comprising the FIG. 25 translation, or a fragment thereof. The invention includes a diagnostic kit comprising a polynucleotide sequence encoding the FIG. 25 translation, or a fragment thereof. The invention includes an immunogenic composition comprising a polypeptide comprising the FIG. 25 translation, or a fragment thereof. The invention includes an antibody which recognizes a polypeptide comprising the FIG. 25 sequence, or a fragment thereof. The FIG. 25 sequence demonstrates functional homology with ORF1b of coronaviruses.

SEQ ID NO: 7188 is an open reading frame within FIG. 25. The invention includes a polypeptide comprising SEQ ID NO: 7188. The invention includes a polypeptide comprising an amino acid sequence having sequence identity to SEQ ID NO: 7188. The invention includes a fragment of a polypeptide comprising SEQ ID NO: 7188. The invention includes a diagnostic kit comprising a polypeptide comprising SEQ ID NO: 7188, or a fragment thereof. The invention includes a diagnostic kit comprising a polynucleotide sequence encoding SEQ ID NO: 7188, or a fragment thereof. The invention includes an immunogenic composition comprising a polypeptide comprising SEQ ID NO: 7188, or a fragment thereof. The invention includes an antibody which recognizes a polypeptide comprising SEQ ID NO: 7188, or a fragment thereof.

SEQ ID NO: 7190 is an open reading frame within SEQ ID NO: 7188. The invention includes a polypeptide comprising SEQ ID NO: 7190, a fragment thereof or a polypeptide having sequence identity thereto. The invention further includes a polynucleotide encoding SEQ ID NO: 7190, a fragment thereof or a polypeptide sequence having sequence identity thereto. An example of a polynucleotide encoding SEQ ID NO: 7190 is given as SEQ ID NO: 7191.

SEQ ID NO: 7188 also contains an open reading frame comprising SEQ ID NO: 6042. The invention includes a polypeptide comprising SEQ ID NO: 6042. The invention includes a polypeptide comprising an amino acid sequence having sequence identity to SEQ ID NO: 6042. The invention includes a fragment of a polypeptide comprising SEQ ID NO: 6042. The invention includes a diagnostic kit comprising a polypeptide comprising SEQ ID NO: 6042, or a fragment thereof. The invention includes a diagnostic kit comprising a polynucleotide sequence encoding SEQ ID NO: 6042, or a fragment thereof. The invention includes an immunogenic composition comprising a polypeptide comprising SEQ ID NO: 6042, or a fragment thereof. The invention includes an antibody which recognizes a polypeptide comprising SEQ ID NO: 6042, or a fragment thereof. SEQ ID NO: 6042 demonstrates functional homology to a coronavirus spike protein.

Predicted transmembrane regions of SEQ ID NO: 6042 are identified below.

Predicted Transmembrane Helices of SEQ ID NO: 6042

The sequence positions in brackets denominate the core region.

Only scores above 500 are considered significant.

| | | | |
|---|---|---|---|
| from | to | score | center |

Inside to outside helices: 18 found

| | | | |
|---|---|---|---|
| 1 (1) | 16 (16) | 959 | 9 |
| 233 (237) | 257 (252) | 905 | 244 |
| 345 (347) | 364 (361) | 490 | 354 |
| 345 (354) | 369 (369) | 420 | 362 |
| 497 (497) | 513 (513) | 239 | 506 |
| 573 (573) | 588 (588) | 811 | 580 |
| 645 (648) | 666 (663) | 302 | 656 |
| 690 (696) | 714 (711) | 428 | 704 |
| 857 (860) | 882 (874) | 1508 | 867 |
| 1031 (1031) | 1046 (1046) | 446 | 1039 |
| 1199 (1203) | 1219 (1217) | 2667 | 1210 |

Outside to inside helices: 13 found

| | | | |
|---|---|---|---|
| 1 (1) | 17 (17) | 684 | 10 |
| 222 (222) | 240 (237) | 238 | 229 |
| 244 (247) | 264 (264) | 613 | 254 |
| 349 (355) | 369 (369) | 314 | 362 |
| 496 (496) | 511 (511) | 488 | 503 |
| 573 (573) | 591 (591) | 712 | 581 |
| 650 (652) | 666 (666) | 474 | 659 |
| 674 (679) | 702 (696) | 190 | 686 |
| 691 (696) | 713 (711) | 210 | 704 |
| 866 (868) | 886 (886) | 1172 | 876 |
| 1198 (1201) | 1215 (1215) | 3221 | 1208 |

SEQ ID NO: 6042, the spike protein, is a surface exposed polypeptide. Recombinant expression of a protein can be hindered by hydrophobic transmembrane regions. Accordingly, the invention includes a polypeptide comprising SEQ ID NO: 6042 wherein one or more of the hydrophobic regions identified above is removed. The invention further includes a polynucleotide encoding such a polypeptide. The invention includes recombinantly expressing the protein in a host cell. Primers for amplifying the gene for spike protein and fragments thereof, such as fragments encoding the soluble ectodomain, include SEQ ID NOS: 9753-9763 (Xiao et al. (2003) Biochem Biophys Res Comm 312:1159-1164).

Further characterization of SEQ ID NO: 6042 is set forth below.

```
PSORT --- Prediction of Protein Localization Sites
version 6.4(WWW)
SEQ ID NO: 6042 - 1255 Residues
Species classification: 4
*** Reasoning Step: 1
Preliminary Calculation of ALOM (threshold: 0.5)
        count: 2
        Position of the most N-terminal TMS: 496 at i = 2
MTOP: membrane topology (Hartmann et al.)
        I(middle): 503 Charge diffirence(C − N): 1.0
McG: Examining signal sequence (McGeoch)
        Length of UR:  13
        Peak Value of UR:  3.28
        Net Charge of CR: 0
        Discriminant Score:  8.66
GvH: Examining signal sequence (von Heijne)
        Signal Score (−3.5): 5.94
        Possible cleavage site: 13
>>> Seems to have a cleavable N-term signal seq.
Amino Acid Composition of Predicted Mature Form:
      calculated from 14
ALOM new cnt: 1 ** thrshld changed to −2
Cleavable signal was detected in ALOM?: 0B
ALOM: finding transmembrane regions (Klein et al.)
        count: 1  value: −12.26  threshold: −2.0
```

| | |
|---|---|
| INTEGRAL | Likelihood = −12.26 Transmembrane 1202-1218 (1194-1228) |
| PERIPHERAL | Likelihood = 0.16 |

```
        modified ALOM score:  2.55
>>> Seems to be a Type Ia membrane protein
        The cytoplasmic tail is from 1219 to 1255 (37 Residues)
Rule: vesicular pathway
Rule: vesicular pathway
Rule: vesicular pathway
(14) or uncleavable?
Gavel: Examining the boundary of mitochondrial targeting seq.
          motif at: 14
        Uncleavable? Ipos set to: 24
Discrimination of mitochondrial target seq.:
        positive (2.18)
Rule: vesicular pathway
Rule: vesicular pathway
Rule: vesicular pathway
*** Reasoning Step: 2
KDEL  Count: 0
Checking apolar signal for intramitochondrial sorting
    (Gavel position 24)  from: 1  to: 10  Score: 8.0
SKL motif (signal for peroxisomal protein):
        pos: 964(1255), count: 1 SRL
        SKL score (peroxisome): 0.1
Amino Acid Composition Tendency for Peroxisome: 1.37
        AAC not from the N-term., score modified
Peroxisomal proteins? Status: notclr
        AAC score (peroxisome): 0.079
Amino Acid Composition tendency for lysosomal proteins
        score: 0.39 Status: notclr
GY motif in the tail of typeIa? (lysosomal)
Checking the amount of Basic Residues (nucleus)
Checking the 4 residue pattern for Nuclear Targeting
Checking the 7 residue pattern for Nuclear Targeting
Checking the Robbins & Dingwall consensus (nucleus)
Checking the RNA binding motif (nucleus or cytoplasm)
Nuclear Signal  Status: negative (0.00)
Type Ia is favored for plasma memb. proteins
Checking the NPXY motif..
Checking the YXRF motif..
Checking N-myristoylation..
----- Final Results -----
plasma membrane --- Certainty = 0.460(Affirmative) <succ>
microbody (peroxisome) --- Certainty = 0.171(Affirmative) <succ>
endoplasmic reticulum (membrane) --- Certainty = 0.100(Affirmative) <succ>
endoplasmic reticulum (lumen) --- Certainty = 0.100(Affirmative) <succ>
```

SEQ ID NO: 6042 appears to have a N-terminus signaling region, followed by a surface exposed region, followed by a transmembrane region followed by a C-terminus cytoplasmic domain region. Accordingly, the invention includes an immunogenic, surface exposed fragment of SEQ ID NO: 6042. Preferably, said fragment comprises an amino acid sequence which does not include the last 50 amino acids of the C-terminus of SEQ ID NO: 6042. Preferably, said fragment comprises an amino acid sequence which does not include the last 70 amino acids of the C-terminus of SEQ ID NO: 6042. Preferably, said fragment does not include a transdomain region of SEQ ID NO: 6042. Preferably, said fragment does not include a C-terminus cytoplasmic domain of SEQ ID NO: 6042. Preferably, said fragment does not include a N-terminus signal sequence. Preferably, said fragment does not include amino acids 1-10 of the N-terminus of SEQ ID NO: 6042. Preferably, said fragment does not include amino acids 1-14 of the N-terminus of SEQ ID NO: 6042. Two oligopeptide fragments of SEQ ID NO: 6042 that are able to elicit anti-spike antibodies are SEQ ID NOS: 7398 & 7399, as described (with additional C-terminus cysteines) by Xiao et al. (2003) Biochem Biophys Res Comm 312:1159-1164. C-terminal truncations of spike protein, with removal of part of the cytoplasmic region, or removal up to and including the transmembrane region, are described by Yang et al. (2004) Nature 428:561-564.

A variant of SEQ ID NO: 6042 that is included within the invention is SEQ ID NO: 9962. Compared to SEQ ID NO: 6042, this sequence has Ser at residue 581 instead of Ala, and has Phe at residue 1152 instead of Leu.

The spike protein of coronaviruses may be cleaved into two separate chains into S1 and S2. The chains may remain associated together to form a dimer or a trimer. Accordingly, the invention includes a polypeptide comprising SEQ ID NO: 6042 wherein said polypeptide has been cleaved into S1 and S2 domains. The invention further includes a polypeptide comprising SEQ ID NO: 6042 wherein amino acids 1-10, preferably amino acids 1-14 of the N-terminus are removed and further wherein SEQ ID NO: 6042 is cleaved into S1 and S2 domains. Preferably the polypeptide is in the form of a trimer.

The spike protein appears to form an alpha-helical structure in the transmembrane region of the protein, preferably in the S2 domain. This alpha-helical structure is thought to associate with at least two additional spike proteins to form a trimer. Helical or coiled regions of the spike protein are identified below. Predicted coiled-coils of SEQ ID NO: 6042 (spike protein) are at amino acids 900-1005 and 1151-1185 (see FIG. 12).

Accordingly, the invention comprises a polypeptide sequence comprising a fragment of SEQ ID NO: 6042 wherein said fragment includes a coiled region of SEQ ID NO: 6042. Said fragment preferably includes the amino acid sequences selected from the group consisting of amino acid positions 900 to 1005 and amino acid positions 1151 to 1185 of SEQ ID NO: 6042. The invention comprises a polypeptide sequence comprising a fragment of SEQ ID NO: 6042, wherein said fragment does not include a coiled region of SEQ ID NO: 6042. Said fragment preferably includes the amino acid sequences selected from the group consisting of amino acid positions 900 to 1005 and amino acid positions 1151 and 1185 of SEQ ID NO: 6042.

The spike protein is believed to play an integral role in fusion and infection of Coronaviruses with mammalian host cells. Analysis of coronavirus spike proteins as well as similar surface proteins in other viruses has identified at least two structural motifs, typically located within the S2 domain, associated with this fusion event: heptad repeats (HR) and membrane fusion peptides.

At least two 4,3 hydrophobic heptad repeat (HR) domains are typically found in the ectodomain of the S2 domain of Coronaviruses. One heptad repeat region (HR1) is typically located adjacent to a fusion peptide while a second heptad region (HR2) is typically located near the C-terminus of the S2 domain, close to the transmembrane anchor. Heptad repeats are characteristic of coiled-coil structures and the heptad repeats found in viral surface proteins (such as coronavirus spike protein) are thought to form bundled helix structures which are involved in viral entry. See Bosch et al., J. Virology (2003) 77:8801-8811 (FIG. 1B of this reference illustrates an alignment of the HR1 and HR2 regions of five coronaviruses along with SARS, annotated “HCov-SARS”).

Heptad repeats generally contain a repeating structure of seven amino acids, designated a-b-c-d-e-f-g, where hydrophobic sidechains of residues a and d typically form an apolar stripe, and electrostatic interactions are found in residues e and g. Position a is most frequently Leu, Ile or Ala and position d is usually Leu or Ala. Residues e and g are often Glu or Gln, with Arg and Lys also prominent at position g. Charged residues are common to positions b, c and f as these residues may be in contact with solvent. Exceptions to these general parameters are known. For instance Pro residues are sometimes found within the heptad.

The HR1 and HR2 sequences of an MHV strain have been postulated to assemble into a thermostable, oligomeric, alphahelical rold-like complex, with the HR1 and HR2 helices oriented in an antiparallel manner. Id. In this same study, HR2 was asserted to be a strong inhitibor of both virus entry into the cell and cell-cell fusion.

HR1 and HR2 sequences have been identified in the SARS virus genome. The SARS virus HR1 region comprises approximately amino acids 879 to 1005 of SEQ ID NO: 6042 or fragments thereof capable of forming at least one alpha-helical turn. Preferably, said fragments comprise at least 7 (e.g., at least 14, 21, 28, 35, 42, 49 or 56) amino acid residues. SEQ ID NO: 7192, includes amino acids 879 to 1005 of SEQ ID NO: 6042.

A preferred fragment of HR1 comprises amino acid residues 879 to 980 of SEQ ID NO: 6042. This preferred fragment is SEQ ID NO: 7193.

Another preferred fragment of HR1 comprises amino acid residues 901 to 1005 of SEQ ID NO: 6042. This preferred fragment is SEQ ID NO: 7194.

The SARS virus HR2 region comprises approximately amino acids 1144 to 1201 of SEQ ID NO: 6042, or fragments thereof capable of forming at least one alpha-helical turn. Preferably, said fragments comprise at least 7 (e.g., at least 14, 21, 28, 35, 42, 49 or 56) amino acid residues. SEQ ID NO: 7195 includes amino acids 1144 to 1201. A preferred fragment of HR2 comprises amino acids 1144 to 1195 of SEQ ID NO: 6042. This preferred fragment is SEQ ID NO: 7196.

Membrane Fusion peptides sequences within the spike protein are also believed to participate in fusion (and infection) of the virus with a host cell. Fusion peptides generally comprise about 16 to 26 amino acid residues which are conserved within viral families. These Membrane Fusion peptides are relatively hydrophobic and generally show an asymmetric distribution of hydrophobitiy when modeled into an alpha helix. They are also generally rich in alanine and glycine.

At least three hydrophobic Membrane Fusion peptide regions have been identified within coronaviruses (PEP1, PEP2, and PEP3). See, Luo et al., “Roles in Cell-Cell Fusion of Two Conserved Hydrophobic Regions in the Murine Coronavirus Spike Protein”, Virology (1998) 244:483-494. FIG. 1 of this paper shows an alignment of Membrane Fusion peptide sequences of Mouse Hepatitis Viris, Bovine Corona Virus, Feline Infectious Peritonitis Virus, Transmissible Gastroenteritis Virus and Infectious Bronchitis Virus. See also, Bosch et al., “The Coronavirus Spike Protein is a Class I Virus Fusion Protein: Structural and Functional Characterization of the Fusion Core Complex” Journal of Virology (2003) 77(16):8801-8811.

PEP1 (SEQ ID NO: 7197), PEP2 (SEQ ID NO: 7198) and PEP3 (SEQ ID NO: 7199) sequences within the SARS spike protein have been identified.

The coronavirus spike proteins (and other similar surface viral proteins) are thought to undergo a conformational change upon receptor binding to the target cell membrane. One or more of the hydrophobic Membrane Fusion peptides are thought to become exposed and inserted into the target membrane as a result of this conformational change. The free energy released upon subsequent refolding of the spike protein to its most stable conformation is believed to play a role in the merger of the viral and cellular membranes.

One or more SARS HR sequences, preferably HR2, or a fragment thereof may be used to inhibit viral entry and membrane fusion with a target mammalian host cell. The invention provides a method of inhibiting viral infection comprising administering a composition comprising one or more SARS HR polypeptides or a fragment thereof. Preferably, the composition comprises a SARS HR2 sequence.

In another embodiment, the invention includes a composition comprising a SARS HR1 sequence, or a fragment thereof and a SARS HR2 sequence, or a fragment thereof. The HR1 and HR2 sequences may optionally be associated together in an oligomer. The composition may comprise the intermediate domain sequence between the HR1 and HR2 domains. The use of such an intermediate sequence may facilitate oligomerization or other structural interaction between the HR regions.

HR sequences for use in the invention may be produced recombinantly by methods known in the art. The SARS HR sequences may be modified to facilitate bacterial expression. In particular, the HR sequences may be modified to facilitate transport of the recombinant protein to the surface of the bacterial host cell. For example, leader sequences to a bacterial membrane protein may be added to the N terminus of the recombinant HR sequences. HR sequences for use in the invention may alternatively be produced by chemical synthesis by methods known in the art (see below).

As discussed in more detail later in the specification, Applicants have identified structural similarities between the SARS spike protein and the surface protein of Neisseria meningitidis, NadA (and other similar bacterial adhesion proteins). Another means of facilitating bacterial expression of HR sequences includes the addition of the stalk and/or anchor sequences of a NadA-like protein to the C-terminus of the recombinant HR sequences. Recombinant sequences containing the bacterial anchor sequence may preferably be prepared in outer membrane vesicles (the preparation of which is discussed in more detail later in the application). Recombinant sequences missing the bacterial anchor sequences may be secreted and isolated from the supernatant.

The invention includes a polypeptide sequence comprising a first sequence and a second sequence, wherein said first sequence comprises a leader sequence for a bacterial membrane protein and wherein said second sequence comprises a HR sequence of a coronavirus. Preferably, said first sequence comprises the leader sequence for a bacterial adhesin protein. More preferably, said bacterial adhesion protein is NadA. Preferably said second sequence comprises HR1, HR2 or both. In one embodiment, the second sequence comprises HR1, HR2 and the intermediate domain sequence present in the naturally occrding spike protein. For example, the second sequence may comprise a fragment of a coronavirus spike protein comprising the amino acids starting with the N-terminus of the HR1 region and ending with the C-terminus of the HR2 region.

The invention further includes a polypeptide sequence comprising a first, second, third and fourth sequence, wherein the first sequence comprises a leader sequence for a bacterial membrane protein; wherein said second sequence comprises a HR sequence of a coronavirus; wherein said third sequence comprises a stalk domain of a bacterial adhesion protein; and wherein said fourth sequence comprises an anchor domain of a bacterial adhesion protein. In one embodiment, the first sequence comprising the leader peptide sequence is removed. In another embodiment, the third sequence comprising the stalk domain is removed. In another embodiment, the fourth sequence comprising the anchor domain is removed.

The polypeptide sequences of the above described constructs may be linked together by means known in the art, including, for example, via glycine linkers.

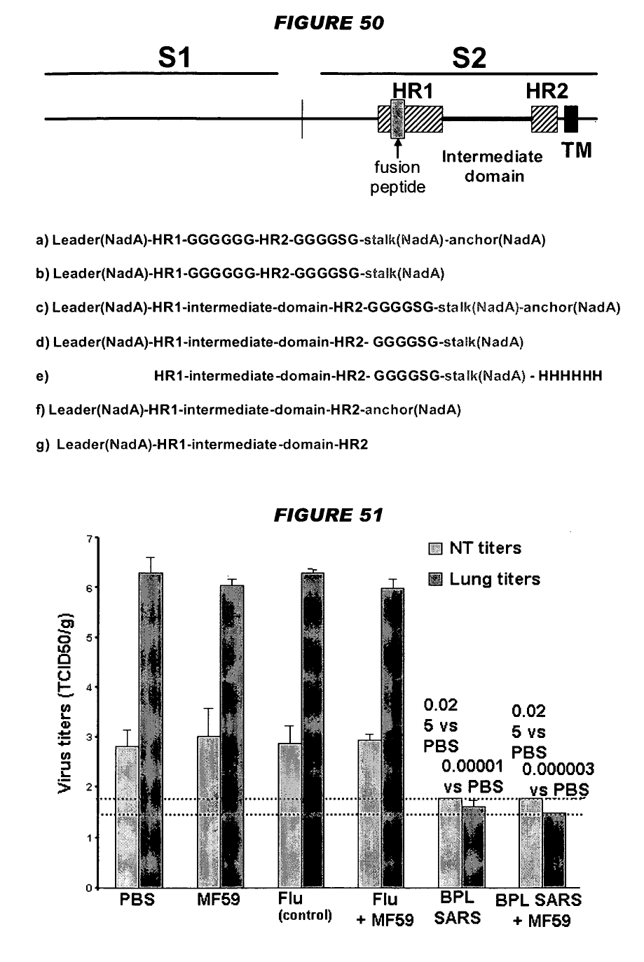

Examples of constructs which may be used in such bacterial expression systems are shown in FIG. 50. Polypeptide sequences of each of the constructs illustrated in FIG. 50 are given as SEQ ID NOS: 7200 to 7206.

7200 Leader NadA (1-29)—HR1 (879-980)—6× gly—HR2 (1144-1195)—stalk+anchor NadA (88-405)

7201 Leader NadA (1-29)—HR1 (879-980)—6× gly—HR2 (1144-1196)—stalk NadA (88-351)

7202 Leader NadA (1-29)—HR1—HR2 (879-1196)—stalk+anchor NadA (88-405)

7203 Leader NadA (1-29)—HR1—HR2 (879-1196)—stalk NadA (88-351)

7204 HR1—HR2 (879-1196)—stalk NadA (88-351)-6× his

7205 Leader NadA (1-29)—HR1—HR2 (879-1196)-anchor NadA (351-405)

7206 Leader NadA (1-29)—HR1—HR2 (879-1196)

Administration of one of more of these Membrane Fusion sequences may also interfere with the ability of a coronavirus to fuse to a host cell membrane. Accordingly, the invention includes an isolated polypeptide comprising an amino acid sequence selected from the group consisting of SEQ ID NO: 7197, SEQ ID NO: 7198 and SEQ ID NO: 7199. The invention further includes an isolated polypeptide comprising an amino acid sequence having sequence homology to an amino acid sequence selected from the group consisting of SEQ ID NO: 7197, SEQ ID NO: 7198 and SEQ ID NO: 7199.

Two or more of these SARS Membrane Fusion peptides can be combined together. The invention includes a composition comprising two SARS Membrane Fusion peptides wherein said peptides are selected from at least two of the amino acids selected from the group consisting of SEQ ID NO: 7197, SEQ ID NO: 7198 and SEQ ID NO: 7199, or a sequence having sequence identity thereto.

Two or more of the SARS Membrane Fusion peptides may be linked together. Accordingly, the invention includes a polypeptide comprising a first amino acid sequence and a second amino acid sequence, wherein said first and second amino acid sequences are selected from the group consisting of SEQ ID NO: 7197, SEQ ID NO: 7198 and SEQ ID NO: 7199, or a sequence having sequence identity thereto. Preferably, said first amino acid sequence and said second amino acid sequence are different SARS Membrane Fusion peptides, i.e., they are not the same.

The invention also includes a method of treating or preventing SARS virus infection comprising administering one or more of the SARS Membrane Fusion peptide compositions described above.

As discussed above, the spike protein is capable of forming trimers. The invention further includes a polypeptide comprising SEQ ID NO: 6042 in trimeric form. The invention includes a composition comprising at least polypeptides wherein each polypeptide comprises at least the alpha-helical coiled region of a SARS virus spike protein. Preferably, the spike protein comprises SEQ ID NO: 6042 or a fragment thereof.

The invention further includes a composition comprising a SARS virus spike protein or a fragment thereof wherein said protein is associated with a transmembrane and wherein said fragment comprises the alpha-helical region of the SARS virus spike protein. Preferably, the composition comprises at least three SARS virus spike proteins or a fragment thereof, wherein the fragment comprises the alpha-helical region of the SARS virus spike protein.

The invention further includes an antibody which specifically binds to a trimeric form of SARS virus spike proteins. Preferably, the spike protein comprises SEQ ID NO: 6042 or a fragment thereof. The invention includes an antibody which specifically binds to a trimeric form of SARS virus spike proteins wherein said proteins are associated with a transmembrane.

The invention further includes an antibody which specifically binds to a monomeric form of SARS virus spike protein or a fragment thereof. Preferably, the antibody specifically binds to a monomeric form of SEQ ID NO: 6042 or a fragment thereof.

The invention further includes a small molecule which interferes with or disrupts the coiling of a SARS viral spike protein trimer.

The invention further includes an attenuated SARS virus for use as a vaccine wherein said attenuated virus contains a polynucleotide insertion, deletion or substitution which does not disrupt the trimeric conformation of the SARS virus spike protein. The invention further includes an attenuated SARS virus for use as a vaccine wherein said attenuated virus contains a polynucleotide insertion, deletion or substitution which does not disrupt the alpha-helical formation of the SARS virus spike protein.

The spike protein may be recombinantly produced. In one embodiment, the spike protein is expressed in virus like particles so that the protein is attached to a cell membrane. Such attachment may facilitate presentation of immunogenic epitopes of the spike protein. Preferably, the alpha-helical portion of the spike protein is associated with the cell membrane. Preferably, the spike proteins form a trimer within the transmembrane region of attachment.

Predicted N-glycosylation sites of SEQ ID NO: 6042 are identified below:

| | | | | Jury | NGlyc |
|---|---|---|---|---|---|
| Position | | | Potential | agreement | result |
| 29 | NYTQ | SEQ ID NO: 7207 | 0.7751 | (9/9) | +++ |
| 65 | NVTG | SEQ ID NO: 7208 | 0.8090 | (9/9) | +++ |
| 109 | NKSQ | SEQ ID NO: 7209 | 0.6081 | (7/9) | + |
| 119 | NSTN | SEQ ID NO: 7210 | 0.7039 | (9/9) | ++ |
| 158 | NCTF | SEQ ID NO: 7211 | 0.5808 | (7/9) | + |
| 227 | NITN | SEQ ID NO: 7212 | 0.7518 | (9/9) | +++ |
| 269 | NGTI | SEQ ID NO: 7213 | 0.6910 | (9/9) | ++ |
| 318 | NITN | SEQ ID NO: 7214 | 0.6414 | (9/9) | ++ |
| 330 | NATK | SEQ ID NO: 7215 | 0.6063 | (8/9) | + |
| 357 | NSTF | SEQ ID NO: 7216 | 0.5746 | (8/9) | + |
| 589 | NASS | SEQ ID NO: 7217 | 0.5778 | (6/9) | + |
| 602 | NCTD | SEQ ID NO: 7218 | 0.6882 | (9/9) | ++ |
| 699 | NFSI | SEQ ID NO: 7219 | 0.5357 | (7/9) | + |
| 783 | NFSQ | SEQ ID NO: 7220 | 0.6348 | (9/9) | ++ |
| 1080 | NGTS | SEQ ID NO: 7221 | 0.5806 | (7/9) | + |
| 1116 | NNTV | SEQ ID NO: 7222 | 0.5106 | (5/9) | + |
| 1176 | NESL | SEQ ID NO: 7223 | 0.6796 | (9/9) | ++ |

Accordingly, the invention includes a polypeptide comprising a fragment of SEQ ID NO: 6042 wherein said fragment comprises one or more of the glycosylation sites identified above (SEQ ID NOS: 7207-7223). The invention further includes a polynucleotide encoding one or more of the fragments identified above. This glycosylation site can be covalently attached to a saccharide. Accordingly, the invention includes a polypeptide comprising a fragment of SEQ ID NO: 6042 wherein said fragment comprises one or more of the glycosylation sites identified above and wherein said polypeptide is glycosylated at one or more of the sites identified above.

Predicted O-glycosylation sites are identified below:

| Residue No. | | Potential | Threshold | Assignment |
|---|---|---|---|---|
| Thr | 698 | 0.8922 | 0.7696 | T |
| Thr | 706 | 0.9598 | 0.7870 | T |
| Thr | 922 | 0.9141 | 0.7338 | T |
| Ser | 36 | 0.8906 | 0.7264 | S |
| Ser | 703 | 0.8412 | 0.7676 | S |

The invention includes a polypeptide comprising a fragment of SEQ ID NO: 6042 wherein said fragment comprises one or more of the O-glycosylation sites identified above. The invention further includes a polynucleotide encoding one or more of the fragments identified above. The invention further includes a polypeptide comprising a fragment of SEQ ID NO: 6042 wherein said fragment comprises one or more of the O-glycosylation sites identified above and further wherein the polypeptide is covalently bonded to a saccharide at one or more of the included glycosylation sites.

The invention further includes a polypeptide comprising a fragment of SEQ ID NO: 6042 wherein said fragment comprises one or more of the N-glycosylation sites identified above and further wherein said fragment comprises one or more of the O-glycosylation sites identified above.

The invention includes a polypeptide comprising a fragment of SEQ ID NO: 6042 wherein said fragment does not include one or more of the glycosylation sites identified above. The invention also includes a polynucleotide encoding such a polypeptide.

Predicted phosphorylation sites of SEQ ID NO: 6042 are Ser-346, Tyr-195, and Tyr-723. Accordingly, the invention comprises a polypeptide comprising a fragment of SEQ ID NO: 6042 wherein said fragment comprises at least ten amino acid residues and wherein said fragment comprises one or more of the amino acids selected from the group consisting of Ser-346, Tyr-195, and Tyr-723. In one embodiment, one or more of the amino acids selected from the group consisting of Ser-346, Tyr-195, and Tyr-723 are phosphorylated.

Expression and functional characterization of the spike glycoprotein has been described by Xiao et al. (2003) Biochem Biophys Res Comm 312:1159-1164.

T-epitopes for SEQ ID NO: 6042 are identified in Table 16. The invention includes a polypeptide for use as an antigen, wherein the polypeptide comprises: (a) an amino acid sequence selected from the group consisting of the T-epitope sequences identified as SEQ ID NOS: 8041-8280; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 8041-8280, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus. The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a polypeptide comprising SEQ ID NO: 6040. The invention includes a polypeptide comprising an amino acid sequence having sequence identity to SEQ ID NO: 6040. The invention includes a fragment of a polypeptide comprising SEQ ID NO: 6040. The invention includes a polynucleotide encoding SEQ ID NO: 6040 or a fragment thereof. The invention includes a diagnostic kit comprising a polypeptide comprising SEQ ID NO: 6040 or a fragment thereof. The invention includes a diagnostic kit comprising a polynucleotide sequence encoding SEQ ID NO: 6040 or a fragment thereof. The invention includes an antibody which recognizes a polypeptide comprising SEQ ID NO: 6040 or a fragment thereof.

SEQ ID NO: 6040 demonstrates functional homology with a membrane protein of coronaviruses. Predicted transmembrane helices of SEQ ID NO: 6040 are identified below:

Predicted Transmembrane Helices

The sequence positions in brackets denominate the core region.

Only scores above 500 are considered significant.

| | | | |
|---|---|---|---|
| from | to | score | center |

Inside to outside helices: 3 found

| | | | |
|---|---|---|---|
| 27 (30) | 48 (45) | 1138 | 38 |
| 137 (139) | 153 (153) | 486 | 146 |

Outside to inside helices: 3 found

| | | | |
|---|---|---|---|
| 28 (31) | 45 (45) | 819 | 38 |
| 71 (73) | 90 (90) | 210 | 81 |
| 136 (142) | 156 (156) | 272 | 149 |

The amino acid region with the highest predicted transmembrane helical region is from amino acid position 27 to 48 of SEQ ID NO: 6040. Such transmembrane regions are often difficult to express recombinantly. Accordingly, the invention includes a polypeptide comprising a fragment of SEQ ID NO: 6040 wherein said fragment does not include the amino acid sequence between positions 27 to 48. The invention includes a polypeptide comprising a fragment of SEQ ID NO: 6040 wherein said fragment does not include the amino acid sequence between positions 28 to 45. The invention also includes a polynucleotide sequence encoding any of the above-identified polypeptides.

SEQ ID NO: 6040 is predicted to be a hypothetical protein of the SARS virus. A prediction of the protein localization of SEQ ID NO: 6040 is set forth below. SEQ ID NO: 6040 is predicted to be located in one of the following locations: mitochondrial matrix space, microbody (peroxisome), nucleus, and mitochondrial inner membrane. SEQ ID NO: 6040 is predicted to be associated with an organelle inside an infected cell.

Accordingly, SEQ ID NO: 6040 is a target for screening of chemical inhibitors to the SARS virus. The invention includes a polypeptide comprises SEQ ID NO: 6040 or a fragment thereof. The invention includes a polynucleotide encoding the polypeptide sequence of SEQ ID NO: 6040 or a fragment thereof. The invention includes a method of screening SEQ ID NO: 6040 for an inhibitor. The invention includes the recombinant expression of SEQ ID NO: 6040 in a host cell. The invention includes a small molecule which prevents the polypeptide of SEQ ID NO: 6040 from associating with an organelle inside of an infected cell. The invention includes a fusion protein wherein said fusion protein comprises SEQ ID NO: 6040.

```
PSORT --- Prediction of Protein Localization Sites
                                    version 6.4(WWW)
SEQ ID NO: 6040  163 Residues
Species classification: 4
*** Reasoning Step: 1
Preliminary Calculation of ALOM (threshold: 0.5)
        count: 0
McG: Examining signal sequence (McGeoch)
        Length of UR:  9
        Peak Value of UR:  1.75
        Net Charge of CR: 1
        Discriminant Score:  −2.56
GvH: Examining signal sequence (von Heijne)
        Signal Score (−3.5): 1.94
        Possible cleavage site: 53
>>> Seems to have no N-terminal signal seq.
Amino Acid Composition of Predicted Mature Form:
      calculated from 1
ALOM new cnt: 0 ** thrshld changed to −2
Cleavable signal was detected in ALOM?: 0B
ALOM: finding transmembrane regions (Klein et al.)
        count: 0  value: 1.32  threshold: −2.0
        PERIPHERAL  Likelihood = 1.32
        modified ALOM score:  −1.16
Gavel: Examining the boundary of mitochondrial targeting seq.
          motif at: 156
        HRSVTI
Discrimination of mitochondrial target seq.:
        notclr (0.88)
Rule: mitochondrial protein
Rule: mitochondrial protein
Rule: mitochondrial protein
Rule: mitochondrial protein
*** Reasoning Step: 2
KDEL  Count: 0
Checking apolar signal for intramitochondrial sorting
    (Gavel position 156)  from: 27  to: 44  Score: 5.0
Mitochondrial matrix? Score: 0.36
SKL motif (signal for peroxisomal protein):
        pos: 99(163), count: 1 SKL
        SKL score (peroxisome): 0.3
Amino Acid Composition Tendency for Peroxisome: −4.28
Peroxisomal proteins? Status: notclr
Amino Acid Composition tendency for lysosomal proteins
        score: 0.02 Status: notclr
Modified score for lysosome: 0.152
Checking the amount of Basic Residues (nucleus)
Checking the 4 residue pattern for Nuclear Targeting
        Found: pos: 132 (5) KRKR
Checking the 7 residue pattern for Nuclear Targeting
Checking the Robbins & Dingwall consensus (nucleus)
Checking the RNA binding motif (nucleus or cytoplasm)
nuc modified. Score: 0.60
Nuclear Signal Status: notclr (0.30)
Checking CaaX motif..
Checking N-myristoylation..
Checking CaaX motif..
----- Final Results -----
mitochondrial matrix space --- Certainty = 0.480(Affirmative) <succ>
microbody (peroxisome) --- Certainty = 0.300(Affirmative) <succ>
nucleus --- Certainty = 0.300(Affirmative) <succ>
mitochondrial inner membrane --- Certainty = 0.188(Affirmative) <succ>
```

Predicted N-glycosylation sites of SEQ ID NO: 6040 are identified below.

| | | | | Jury | NGlyc |
|---|---|---|---|---|---|
| Position | | | Potential | agreement | result |
| 2 | NKTG | (SEQ ID NO: 7255) | 0.7804 | (9/9) | +++ |
| 106 | NLTL | (SEQ ID NO: 7256) | 0.6123 | (7/9) | + |

Accordingly, the invention comprises a fragment of SEQ ID NO: 6040 wherein said fragment is at least ten amino acids and wherein said fragment comprises one or more of the asparagines from the amino acid positions of SEQ ID NO: 6040 selected from the group consisting of 2 and 106. The invention includes a fragment of SEQ ID NO: 6040 wherein said fragment comprises one or more amino acid sequences selected from the group consisting of SEQ ID NO: 7255 and SEQ ID NO: 7256. Preferably, the fragment comprises the amino acid sequence NKTG (SEQ ID NO: 7255).

The invention includes a polypeptide comprising a fragment of SEQ ID NO: 6040 wherein said fragment does not include one or more of the glycosylation sites identified above. The invention also includes a polynucleotide encoding such a polypeptide.

T-epitopes for SEQ ID NO: 6040 are identified in Table 14. The invention includes a polypeptide for use as an antigen, wherein the polypeptide comprises: (a) an amino acid sequence selected from the group consisting of the T-epitope sequences identified in SEQ ID NOS: 7640-7800; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 7640-7800, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus.

The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a polypeptide comprising SEQ ID NO: 6041. SEQ ID NO: 6041 demonstrates functional homology with a portion of an ORF 1ab polyprotein. The invention includes a polypeptide comprising an amino acid sequence having sequence identity to SEQ ID NO: 6041. The invention includes a fragment of a polypeptide comprising SEQ ID NO: 6041. The invention includes a polynucleotide sequence encoding an amino acid sequence having sequence identity to SEQ ID NO: 6041. The invention includes a polynucleotide encoding a fragment of a polypeptide comprising SEQ ID NO: 6041.

The invention includes a diagnostic kit comprising a polypeptide comprising SEQ ID NO: 6041 or a fragment therof. The invention includes a diagnostic kit comprising a polynucleotide encoding SEQ ID NO: 6041 or a fragment thereof. The invention includes an immunogenic composition comprising a polypeptide comprising SEQ ID NO: 6041 or a fragment thereof. The invention includes an antibody which recognizes a polypeptide comprising SEQ ID NO: 6041 or a fragment thereof.

The polyproteins of coronaviruses are associated with enzymatic activity. Accordingly, SEQ ID NO: 6041 is a target for screening of chemical inhibitors to the SARS virus. The invention includes a polypeptide comprising SEQ ID NO: 6041 or a fragment thereof. The invention includes a polynucleotide encoding the polypeptide sequence of SEQ ID NO: 6041 or a fragment thereof. The invention includes a method of screening SEQ ID NO: 6041 for an inhibitor. The invention includes the recombinant expression of SEQ ID NO: 6041 in a host cell. The invention includes a small molecule which prevents the polypeptide of SEQ ID NO: 6041 from performing enzymative activity. The invention includes a fusion protein wherein said fusion protein comprises SEQ ID NO: 6041.

Predicted transmembrane or hydrophobic regions of SEQ ID NO: 6041 are identified below. Although the polyprotein of coronaviruses is proteolytically cleaved into numerous smaller proteins, hydrophobic domains in the polyprotein are known to mediate the membrane association of the replication complex and to be able to dramatically alter the architecture of host cell membranes. Accordingly, the hydrophobic domains of the polyprotein are targets for genetic mutation to develop attenuated SARS virus vaccines. The hydrophobic domains are also targets for small molecule inhibitors of the SARS virus. The hydrophobic domains may also be used to generate antibodies specific to those regions to treat or prevent SARS virus infection.

Possible Transmembrane Helices of SEQ ID NO: 6041

The sequence positions in brackets denominate the core region.

Only scores above 500 are considered significant.

| | | | |
|---|---|---|---|
| from | to | score | center |

Inside to outside helices: 18 found

| | | | |
|---|---|---|---|
| 234 (234) | 254 (250) | 1046 | 241 |
| 256 (256) | 272 (270) | 252 | 263 |
| 319 (319) | 334 (334) | 227 | 327 |
| 503 (505) | 522 (519) | 405 | 512 |
| 613 (615) | 633 (629) | 619 | 622 |
| 677 (679) | 703 (696) | 467 | 689 |
| 849 (851) | 869 (865) | 229 | 858 |
| 1080 (1080) | 1097 (1094) | 306 | 1087 |
| 1147 (1149) | 1163 (1163) | 354 | 1156 |
| 1557 (1557) | 1581 (1577) | 817 | 1567 |
| 1954 (1954) | 1971 (1971) | 832 | 1964 |
| 2369 (2372) | 2395 (2387) | 300 | 2379 |
| 2513 (2513) | 2532 (2529) | 690 | 2522 |

Outside to inside helices: 14 found

| | | | |
|---|---|---|---|
| 239 (239) | 254 (254) | 924 | 247 |
| 239 (248) | 272 (263) | 468 | 256 |
| 311 (314) | 334 (328) | 267 | 321 |
| 499 (503) | 522 (519) | 485 | 512 |
| 617 (617) | 634 (631) | 425 | 624 |
| 849 (853) | 872 (872) | 572 | 864 |
| 1147 (1147) | 1162 (1162) | 765 | 1155 |
| 1564 (1564) | 1581 (1579) | 883 | 1572 |
| 1951 (1951) | 1968 (1966) | 657 | 1958 |
| 2513 (2522) | 2539 (2537) | 711 | 2529 |

Accordingly, the invention includes a polypeptide comprising a fragment of SEQ ID NO: 6041, wherein said fragment comprises an amino acid sequence including one or more of the hydrophobic transmembrane sequences identified above. The invention includes a polypeptide comprising a fragment of SEQ ID NO: 6041 wherein said fragment comprises one or more of the following polypeptide sequences of SEQ ID NO: 6041: 234-254, 613-633, 1557-1581, 1954-1971, 2513-2532, 239-254, 1564-1581, 1951-1968, 2513-2539. Preferably, the fragment comprises one or more of the following polypeptide sequences of SEQ ID NO: 6041: 234-254 and 239-254. The invention also includes polynucleotides encoding each of the polypeptide fragments identified above.

The invention includes an attenuated SARS virus wherein said attenuated SARS virus contains an addition, deletion or substitution in the polynucleotides encoding for one of the hydrophobic domains identified above. The invention also includes a method for creating an attenuated SARS virus comprising mutating a SARS virus by adding, deleting or substituting the viral genome of the SARS virus to alter the coding of one or more of the hydrophobic domains of SEQ ID NO: 6041 identified above.

The invention includes an antibody which specifically identifies one or more of the hydrophobic regions of SEQ ID NO: 6041 identified above. The invention includes a small molecule which binds to, interferes with the hydrophobicity of or otherwise disrupts one or more of the hydrophobic regions of SEQ ID NO: 6041 identified above.

Predicted N-glycosylation sites of SEQ ID NO: 6041 are identified below:

| | | | | Jury | NGlyc |
|---|---|---|---|---|---|
| Position | | | Potential | agreement | result |
| 571 | NLSH | (SEQ ID NO: 7257) | 0.6598 | (8/9) | + |
| 835 | NTSR | (SEQ ID NO: 7258) | 0.5762 | (7/9) | + |
| 958 | NVTD | (SEQ ID NO: 7259) | 0.7494 | (9/9) | ++ |
| 1113 | NISD | (SEQ ID NO: 7260) | 0.7259 | (8/9) | + |
| 1205 | NSTL | (SEQ ID NO: 7261) | 0.6296 | (9/9) | ++ |
| 1460 | NVTG | (SEQ ID NO: 7262) | 0.6844 | (9/9) | ++ |
| 1685 | NHSV | (SEQ ID NO: 7263) | 0.5181 | (5/9) | + |
| 2029 | NKTT | (SEQ ID NO: 7264) | 0.5423 | (5/9) | + |

Accordingly, the invention comprises a polypeptide comprising a fragment of the amino acid sequence of SEQ ID NO: 6041, wherein said fragment comprises one or more of the N-glycosylation sites identified above. The invention comprises a polypeptide comprising a fragment of SEQ ID NO: 6041 wherein said fragment comprises one or more of sequences SEQ ID NOS: 7257-7264. Preferably, the fragment comprises one or more of the sequences SEQ ID NOS: 7257, 7259, 7260, 7261 and 7262. The invention further comprises a polynucleotide encoding one or more of the polypeptides identified above.

The invention includes a polypeptide comprising a fragment of SEQ ID NO: 6041 wherein said fragment does not include one or more of the glycosylation sites identified above. The invention also includes a polynucleotide encoding such a polypeptide.

T-epitopes for SEQ ID NO: 6041 are identified in Table 15. The invention includes a polypeptide for use as an antigen, wherein the polypeptide comprises: (a) an amino acid sequence selected from the group consisting of the T-epitope sequences identified in SEQ ID NOS: 7801-8040; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 7801-8040, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus.

The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a polypeptide sequence SEQ ID NO: 6043 or a fragment thereof. The invention includes a polypeptide comprising an amino acid sequence having sequence identity to SEQ ID NO: 6043. The invention includes a polynucleotide sequence encoding the amino acid sequence of SEQ ID NO: 6043 or a fragment thereof.

Predicted transmembrane regions of SEQ ID NO: 6043 are set forth below.

| | | | |
|---|---|---|---|
| from | to | score | center |

Inside to outside helices: 4 found

| | | | |
|---|---|---|---|
| 41 (41) | 56 (56) | 1789 | 49 |
| 76 (79) | 99 (99) | 2142 | 89 |
| 105 (105) | 125 (125) | 1250 | 115 |

Outside to inside helices: 3 found

| | | | |
|---|---|---|---|
| 41 (41) | 59 (56) | 2053 | 49 |
| 76 (82) | 98 (96) | 1580 | 89 |
| 103 (105) | 125 (123) | 1257 | 115 |

The amino acid region with the highest predicted transmembrane helical region is from amino acid position 76 to 99 of SEQ ID NO: 6043. Such transmembrane regions are often difficult to express recombinantly. Accordingly, the invention includes a polypeptide comprising a fragment of SEQ ID NO: 6043 wherein said fragment does not include one or more of the hydrophobic amino acid sequences identified above. Preferably, the fragment does not include the amino acids between positions 27 to 48. The invention also includes a polynucleotide sequence encoding any of the above-identified polypeptides.

SEQ ID NO: 6043 is predicted to be a hypothetical protein of the SARS virus. A prediction of the protein localization of SEQ ID NO: 6043 is set forth below. SEQ ID NO: 6043 is predicted to be located in one of the following locations: mitochondrial inner membrane, plasma membrane, Golgi body, and mitochondrial intermembrane space. SEQ ID NO: 6043 may be associated with an organelle inside an infected cell.

Accordingly, SEQ ID NO: 6043 is a target for screening of chemical inhibitors to the SARS virus. The invention includes a polypeptide comprises SEQ ID NO: 6043 or a fragment thereof. The invention includes a polynucleotide encoding the polypeptide sequence of SEQ ID NO: 6043 or a fragment thereof. The invention includes a method of screening SEQ ID NO: 6043 for an inhibitor. The invention includes the recombinant expression of SEQ ID NO: 6043 in a host cell. The invention includes a small molecule which prevents the polypeptide of SEQ ID NO: 6043 from associating with an organelle inside of an infected cell. The invention includes a fusion protein wherein said fusion protein comprises SEQ ID NO: 6043.

```
PSORT --- Prediction of Protein Localization Sites for SEQ ID NO: 6043
                                        version 6.4(WWW)
Species classification: 4
*** Reasoning Step: 1
Preliminary Calculation of ALOM (threshold: 0.5)
        count: 3
        Position of the most N-terminal TMS: 40 at i = 2
MTOP: membrane topology (Hartmann et al.)
        I(middle): 47  Charge diffirence(C − N): 3.5
McG: Examining signal sequence (McGeoch)
        Length of UR:  12
        Peak Value of UR:  1.41
        Net Charge of CR: 0
        Discriminant Score:  −4.67
GvH: Examining signal sequence (von Heijne)
        Signal Score (−3.5): 3.44
        Possible cleavage site: 15
>>> Seems to have no N-terminal signal seq.
Amino Acid Composition of Predicted Mature Form:
      calculated from 1
ALOM new cnt: 2 ** thrshld changed to −2
Cleavable signal was detected in ALOM?: 0B
ALOM: finding transmembrane regions (Klein et al.)
        count: 2  value: −6.90  threshold: −2.0
```

| | |
|---|---|
| INTEGRAL | Likelihood = −6.90 Transmembrane 83-99 (78-101) |
| INTEGRAL | Likelihood = −5.04 Transmembrane 40-56 (37-60) |
| PERIPHERAL | Likelihood = −0.32 |

```
        modified ALOM score:  1.48
>>> Likely a Type IIIb membrane protein (Nexo Ccyt)
Gavel: Examining the boundary of mitochondrial targeting seq.
          motif at: 128
        MRCWLC
Discrimination of mitochondrial target seq.:
        notclr (0.76)
Rule: mitochondrial protein
Rule: mitochondrial protein
Rule: mitochondrial protein
Rule: mitochondrial protein
*** Reasoning Step: 2
Type IIIa or IIIb is favored for ER memb. proteins
KDEL  Count: 0
Checking apolar signal for intramitochondrial sorting
    (Gavel position 128)  from: 39  to: 56  Score: 11.5
>>> Seems to have an intramitochondrial signal
Mitochondrial inner membrane? Score: 0.59
Mitochondrial intermemb.space? Score: 0.22
SKL motif (signal for peroxisomal protein):
        pos: 92(274), count: 1 SHL
        SKL score (peroxisome):  0.3
Amino Acid Composition Tendency for Peroxisome:  4.78
Peroxisomal proteins? Status: positive
Amino Acid Composition tendency for lysosomal proteins
        score: 1.16 Status: notclr
Type III proteins may be localized at Golgi
Checking the amount of Basic Residues (nucleus)
Checking the 4 residue pattern for Nuclear Targeting
Checking the 7 residue pattern for Nuclear Targeting
Checking the Robbins & Dingwall consensus (nucleus)
Checking the RNA binding motif (nucleus or cytoplasm)
Nuclear Signal  Status: negative (0.00)
Check the Number of TMSs for typeIII (plasma memb.)
Checking N-myristoylation..
----- Final Results -----
mitochondrial inner membrane --- Certainty = 0.664(Affirmative) <succ>
plasma membrane --- Certainty = 0.600(Affirmative) <succ>
Golgi body --- Certainty = 0.400(Affirmative) <succ>
mitochondrial intermembrane space --- Certainty = 0.362(Affirmative) <succ>
```

Predicted N— and O-glycosylation sites of SEQ ID NO: 6043 are identified below.

| | | Jury | NGlyc |
|---|---|---|---|
| Position | Potential | agreement | result |
| 227 NATF (SEQ ID NO: 7265) | 0.6328 | (7/9) | + |

| Residue No. | | Potential | Threshold | Assignment |
|---|---|---|---|---|
| Thr | 28 | 0.9095 | 0.6280 | T |
| Thr | 32 | 0.8740 | 0.6595 | T |
| Thr | 34 | 0.9058 | 0.6655 | T |
| Thr | 170 | 0.6816 | 0.6600 | T |
| Thr | 267 | 0.9240 | 0.5779 | T |
| Thr | 268 | 0.7313 | 0.5708 | T |
| Thr | 269 | 0.9859 | 0.5583 | T |
| Thr | 270 | 0.8023 | 0.5492 | T |
| Ser | 27 | 0.6930 | 0.6091 | S |
| Ser | 252 | 0.6457 | 0.5977 | S |

Accordingly, the invention comprises a polypeptide comprising a fragment of the amino acid sequence of SEQ ID NO: 6043, wherein said fragment comprises the N-glycosylation sites or O-glycosylation sites identified above. The invention comprises a polypeptide comprising a fragment of SEQ ID NO: 6043 wherein said fragment comprises one or more of the N-glycosylation sites or O-glycosylation sites identified above. The invention further comprises a polynucleotide encoding one or more of the polypeptides identified above.

The invention includes a polypeptide comprising a fragment of SEQ ID NO: 6043 wherein said fragment does not include one or more of the glycosylation sites identified above. The invention also includes a polynucleotide encoding such a polypeptide.

T-epitopes for SEQ ID NO: 6043 are identified in Table 17. The invention includes a polypeptide for use as an antigen, wherein the polypeptide comprises: (a) an amino acid sequence selected from the group consisting of the T-epitope sequences identified in SEQ ID NOS: 8281-8486; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 8281-8486, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus. The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a polypeptide comprising SEQ ID NO: 6044. The invention includes a polypeptide comprising a fragment of SEQ ID NO: 6044 or a sequence having sequence identity to SEQ ID NO:206. The invention includes a polynucleotide encoding SEQ ID NO: 6044.

SEQ ID NO: 6044 is identified as a hypothetical protein. Predicted hydrophobic or transmembrane regions of SEQ ID NO: 6044 are identified below:

| | | | |
|---|---|---|---|
| from | to | score | center |

Inside to outside helices: 3 found

| | | | |
|---|---|---|---|
| 1 (1) | 17 (15) | 891 | 8 |
| 47 (47) | 66 (63) | 221 | 56 |

Outside to inside helices: 4 found

| | | | |
|---|---|---|---|
| 1 (4) | 21 (19) | 599 | 11 |

Accordingly, the invention includes a polypeptide comprising a fragment of SEQ ID NO: 6044 wherein said fragment does not include one or more of the hydrophobic amino acid sequences identified above. Preferably, the fragment does not include the amino acids between positions 1 to 19. The invention also includes a polynucleotide sequence encoding any of the above-identified polypeptides.

SEQ ID NO: 6044 is predicted to be a hypothetical protein of SARS virus. A prediction of the protein localization of SEQ ID NO: 6044 is set forth below. SEQ ID NO: 6044 is predicted to be located in one of the following locations: nucleus, mitochondrial matrix, lysosome (lumen), and microbody (peroxisome). SEQ ID NO: 6044 may be associated with an organelle inside an infected cell.

Accordingly, SEQ ID NO: 6044 is a target for screening of chemical inhibitors to the SARS virus. The invention includes a polypeptide comprises SEQ ID NO: 6044 or a fragment thereof The invention includes a polynucleotide encoding the polypeptide sequence of SEQ ID NO: 6044 or a fragment thereof. The invention includes a method of screening SEQ ID NO: 6044 for an inhibitor. The invention includes the recombinant expression of SEQ ID NO: 6044 in a host cell. The invention includes a small molecule which prevents the polypeptide of SEQ ID NO: 6044 from associating with an organelle inside of an infected cell. The invention includes a fusion protein wherein said fusion protein comprises SEQ ID NO: 6044.

```
PSORT --- Prediction of Protein Localization Sites for SEQ ID NO: 6044
                                version 6.4(WWW)
154 Residues
Species classification: 4
*** Reasoning Step: 1
Preliminary Calculation of ALOM (threshold: 0.5)
        count: 0
McG: Examining signal sequence (McGeoch)
        Length of UR:  7
        Peak Value of UR:  1.06
        Net Charge of CR: 1
        Discriminant Score:  −7.97
GvH: Examining signal sequence (von Heijne)
        Signal Score (−3.5): −3.28
        Possible cleavage site: 34
>>> Seems to have no N-terminal signal seq.
Amino Acid Composition of Predicted Mature Form:
      calculated from 1
ALOM new cnt: 0 ** thrshld changed to −2
Cleavable signal was detected in ALOM?: 0B
ALOM: finding transmembrane regions (Klein et al.)
        count: 0  value:  1.43  threshold: −2.0
        PERIPHERAL  Likelihood = 1.43
        modified ALOM score:  −1.19
Gavel: Examining the boundary of mitochondrial targeting seq.
          motif at: 151
        FRKKQV
Discrimination of mitochondrial target seq.:
        notclr (−0.46)
*** Reasoning Step: 2
KDEL  Count: 0
Checking apolar signal for intramitochondrial sorting
    (Gavel position 151)  from: 46  to: 50  Score: 5.0
Mitochondrial matrix? Score: 0.36
SKL motif (signal for peroxisomal protein):
        pos: −1(154), count: 0
Amino Acid Composition Tendency for Peroxisome: 0.61
Peroxisomal proteins? Status: notclr
        AAC score (peroxisome): 0.149
Amino Acid Composition tendency for lysosomal proteins
        score: 0.81 Status: notclr
Modified score for lysosome: 0.231
Checking the amount of Basic Residues (nucleus)
Checking the 4 residue pattern for Nuclear Targeting
        Found: pos: 134 (3) KHKK
Checking the 7 residue pattern for Nuclear Targeting
Checking the Robbins & Dingwall consensus (nucleus)
        Found: pos: 136 (3) KK VSTNLCTHSF RKKQV
Final Robbins Score (nucleus): 0.60
Checking the RNA binding motif (nucleus or cytoplasm)
nuc modified.  Score: 0.90
Nuclear Signal  Status: positive (0.70)
Checking CaaX motif..
Checking N-myristoylation..
Checking CaaX motif..
----- Final Results -----
nucleus --- Certainty = 0.880(Affirmative) <succ>
mitochondrial matrix space --- Certainty = 0.360(Affirmative) <succ>
lysosome (lumen) --- Certainty = 0.231(Affirmative) <succ>
microbody (peroxisome) --- Certainty = 0.149(Affirmative) <succ>
```

One predicted O-glycosylation site of SEQ ID NO: 6044 is identified at residue 4:

| Residue No. | | Potential | Threshold | Assignment |
|---|---|---|---|---|
| Thr | 4 | 0.6839 | 0.6484 | T |

Accordingly, the invention comprises a polypeptide comprising a fragment of the amino acid sequence of SEQ ID NO: 6044, wherein said fragment comprises the O-glycosylation site identified above. The invention further comprises a polynucleotide encoding one or more of the polypeptides identified above.

The invention includes a polypeptide comprising a fragment of SEQ ID NO: 6044 wherein said fragment does not include one or more of the glycosylation sites identified above. The invention also includes a polynucleotide encoding such a polypeptide.

T-epitopes for SEQ ID NO: 6044 are identified in Table 18. The invention includes a polypeptide for use as an antigen, wherein the polypeptide comprises: (a) an amino acid sequence selected from the group consisting of the T-epitope sequences identified in SEQ ID NOS: 8487-8665; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 8487-8665, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus. The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a polypeptide sequence comprising SEQ ID NO: 6045. The invention includes a polypeptide sequence comprising an amino acid sequence having sequence identity to SEQ ID NO: 6045. The invention includes a polypeptide sequence comprising a fragment of SEQ ID NO: 6045. The invention includes a polynucleotide sequence encoding any of these polypeptides.

SEQ ID NO: 6045 demonstrates functional homology with the envelope or small membrane protein of coronaviruses. The invention includes a diagnostic kit comprising a polypeptide comprising SEQ ID NO: 6045 or a fragment thereof. The invention includes a diagnostic kit comprising a polynucleotide encoding SEQ ID NO: 6045 or a fragment thereof. The invention includes an immunogenic composition comprising SEQ ID NO: 6045 or a fragment thereof. The invention includes an antibody which recognizes a polypeptide comprising SEQ ID NO: 6045 or a fragment thereof.

Predicted transmembrane regions of SEQ ID NO: 6045 are identified below:

| | | | |
|---|---|---|---|
| from | to | score | center |

Inside to outside helices: 1 found

| | | | |
|---|---|---|---|
| 17 (19) | 33 (33) | 2881 | 26 |

Outside to inside helices: 1 found

| | | | |
|---|---|---|---|
| 17 (17) | 34 (34) | 2981 | 27 |

Accordingly, the invention includes a polypeptide comprising a fragment of SEQ ID NO: 6045 wherein said fragment does not include one or more of the hydrophobic amino acid sequences identified above. Preferably, the fragment does not include the amino acids between positions 17 to 34. The invention also includes a polynucleotide sequence encoding any of the above-identified polypeptides. In one embodiment, the invention includes a polypeptide comprising a fragment of SEQ ID NO: 6045 wherein said fragment does not include amino acid residues 1-34 of SEQ ID NO: 6045.

Predicted protein Localization Site of SEQ ID NO: 6045 is below.

```
PSORT --- Prediction of Protein Localization Sites for SEQ ID NO: 6045
                                                version 6.4(WWW)
Species classification: 4
*** Reasoning Step: 1
Preliminary Calculation of ALOM (threshold: 0.5)
        count: 2
        Position of the most N-terminal TMS: 17 at i = 1
MTOP: membrane topology (Hartmann et al.)
        I(middle): 24  Charge diffirence(C-N): 2.0
McG: Examining signal sequence (McGeoch)
        Length of UR: 29
        Peak Value of UR: 3.40
        Net Charge of CR: −2
        Discriminant Score: 13.07
GvH: Examining signal sequence (von Heijne)
        Signal Score (−3.5): 4.37
        Possible cleavage site: 32
... positive value of mtop ...
>>> Seems to have an uncleavable N-term signal seq.
Amino Acid Composition of Predicted Mature Form:
      calculated from 1
ALOM new cnt: 1 ** thrshld changed to −2
Cleavable signal was detected in ALOM?: 0B
ALOM: finding transmembrane regions (Klein et al.)
        count: 1  value: −15.12  threshold: −2.0
```

| | |
|---|---|
| INTEGRAL | Likelihood = −15.12 Transmembrane 17-33 (8-44) |
| PERIPHERAL | Likelihood = 0.47 |

```
        modified ALOM score:  3.12
>>> Seems to be a Type Ib (Nexo Ccyt) membrane protein
        The cytoplasmic tail is from 34 to 76 (44 Residues)
Rule: vesicular pathway
Rule: vesicular pathway
Rule: vesicular pathway
(6) or uncleavable?
Gavel: Examining the boundary of mitochondrial targeting seq.
          motif at: 6
        Uncleavable? Ipos set to: 16
Discrimination of mitochondrial target seq.:
        notclr (0.19)
Rule: vesicular pathway
Rule: vesicular pathway
Rule: vesicular pathway
*** Reasoning Step: 2
> Relative position of the end of the tail: 44%
Memb.protein with uncleavable signl is often at ER
KDEL  Count: 0
Checking apolar signal for intramitochondrial sorting
    (Gavel position 16)  from: 70  to: 99  Score: 21.5
>>> Seems to have an intramitochondrial signal
SKL motif (signal for peroxisomal protein):
        pos: −1(76), count: 0
Amino Acid Composition Tendency for Peroxisome: −4.11
Peroxisomal proteins? Status: negative
Amino Acid Composition tendency for lysosomal proteins
        score: 0.68 Status: notclr
Checking the amount of Basic Residues (nucleus)
Checking the 4 residue pattern for Nuclear Targeting
Checking the 7 residue pattern for Nuclear Targeting
Checking the Robbins & Dingwall consensus (nucleus)
Checking the RNA binding motif (nucleus or cytoplasm)
Nuclear Signal  Status: negative (0.00)
Check cytoplasmic tail for typeIb (plasma memb.)
Checking the NPXY motif..
Checking the YXRF motif..
Checking N-myristoylation..
----- Final Results -----
plasma membrane --- Certainty = 0.730(Affirmative) <succ>
endoplasmic reticulum (membrane) --- Certainty = 0.640(Affirmative) <succ>
endoplasmic reticulum (lumen) --- Certainty = 0.100(Affirmative) <succ>
outside --- Certainty = 0.100(Affirmative) <succ>
```

Predicted N-glycosylation sites of SEQ ID NO: 6045 are identified at residues 48 and 66:

| | | Jury | NGlyc | |
|---|---|---|---|---|
| Position | Potential | agreement | result | |
| 48 NVSL | 0.6514 | (9/9) | ++ | (SEQ ID NO: 7266) |
| 66 NSSE | 0.5880 | (7/9) | + | (SEQ ID NO: 7267) |

Accordingly, the invention comprises a polypeptide comprising a fragment of the amino acid sequence of SEQ ID NO: 6045, wherein said fragment comprises one or more of the N-glycosylation sites identified above. The invention comprises a polypeptide comprising a fragment of SEQ ID NO: 6045 wherein said fragment comprises one or more of the N-glycosylation sites identified above. The invention further comprises a polynucleotide encoding one or more of the polypeptides identified above.

The invention includes a polypeptide comprising a fragment of SEQ ID NO: 6045 wherein said fragment does not include one or more of the glycosylation sites identified above. The invention also includes a polynucleotide encoding such a polypeptide.

T-epitopes for SEQ ID NO: 6045 are identified in Table 19. The invention includes a polypeptide for use as an antigen, wherein the polypeptide comprises: (a) an amino acid sequence selected from the group consisting of the T-epitope sequences identified in SEQ ID NOS: 8666-8820; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 8666-8820, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus. The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a polypeptide sequence comprising SEQ ID NO: 6046. The invention includes polypeptide sequences comprising an amino acid sequence having sequence identity to SEQ ID NO: 6046. The invention includes a polypeptide sequence comprising a fragment of SEQ ID NO: 6046. The invention includes a polynucleotide encoding one of these polypeptides.

SEQ ID NO: 6046 has functional homology with a matrix protein of a coronavirus. The invention includes a diagnostic kit comprising a polypeptide comprising SEQ ID NO: 6046 or a fragment thereof. The invention includes a diagnostic kit comprising a polynucleotide encoding SEQ ID NO: 6046 or a fragment thereof. The invention includes an immunogenic composition comprising SEQ ID NO: 6046 or a fragment thereof. The invention includes an antibody which recognizes a polypeptide comprising SEQ ID NO: 6046 or a fragment thereof.

Predicted transmembrane regions of SEQ ID NO: 6046 are identified below.

| | | | |
|---|---|---|---|
| from | to | score | center |

Inside to outside helices: 3 found

| | | | |
|---|---|---|---|
| 21 (21) | 38 (36) | 2412 | 29 |
| 51 (53) | 69 (69) | 2645 | 60 |
| 74 (82) | 96 (96) | 2464 | 89 |

Outside to inside helices: 3 found

| | | | |
|---|---|---|---|
| 18 (21) | 38 (38) | 2363 | 28 |
| 52 (52) | 67 (67) | 2363 | 60 |
| 76 (76) | 95 (92) | 2605 | 84 |

Accordingly, the invention includes a polypeptide comprising a fragment of SEQ ID NO: 6046 wherein said fragment does not include one or more of the hydrophobic amino acid sequences identified above. Preferably, the fragment does not include the amino acids between positions selected from the group consisting of 18 to 38, 52 to 67 and 76 to 95. The invention also includes a polynucleotide sequence encoding any of the above-identified polypeptides.

Predicted protein localization of SEQ ID NO: 6046 is set forth below.

```
PSORT --- Prediction of Protein Localization Sites
                                        version 6.4(WWW)
Species classification: 4
*** Reasoning Step: 1
Preliminary Calculation of ALOM (threshold: 0.5)
        count: 3
        Position of the most N-terminal TMS: 21 at i = 1
MTOP: membrane topology (Hartmann et al.)
        I(middle): 28  Charge diffirence(C − N): 6.0
McG: Examining signal sequence (McGeoch)
        Length of UR:  1
        Peak Value of UR:  3.16
        Net Charge of CR: −3
        Discriminant Score:  2.21
GvH: Examining signal sequence (von Heijne)
        Signal Score (−3.5): 4.29
        Possible cleavage site: 39
... positive value of mtop ...
>>> Seems to have an uncleavable N-term signal seq.
Amino Acid Composition of Predicted Mature Form:
      calculated from 1
Cleavable signal was detected in ALOM?: 0B
ALOM: finding transmembrane regions (Klein et al.)
        count: 3  value:  −7.64  threshold: 0.5
```

| | |
|---|---|
| INTEGRAL | Likelihood = −7.64 Transmembrane 21-37 (18-39) |
| INTEGRAL | Likelihood = −7.59 Transmembrane 50-66 (43-72) |
| INTEGRAL | Likelihood = −5.04 Transmembrane 79-95 (72-99) |
| PERIPHERAL | Likelihood = 2.38 |

```
        modified ALOM score:  2.13
>>> Likely a Type IIIb membrane protein (Nexo Ccyt)
Rule: vesicular pathway
Rule: vesicular pathway
Rule: vesicular pathway
(2) or uncleavable?
Gavel: Examining the boundary of mitochondrial targeting seq.
          motif at: 2
        Uncleavable? Ipos set to: 12
Discrimination of mitochondrial target seq.:
        negative (−4.16)
Rule: vesicular pathway
Rule: vesicular pathway
Rule: vesicular pathway
*** Reasoning Step: 2
Type IIIa or IIIb is favored for ER memb. proteins
Memb.protein with uncleavable signl is often at ER
KDEL  Count: 0
Checking apolar signal for intramitochondrial sorting
SKL motif (signal for peroxisomal protein):
        pos: −1(221), count: 0
Amino Acid Composition Tendency for Peroxisome:  5.01
Peroxisomal proteins? Status: notclr
Amino Acid Composition tendency for lysosomal proteins
        score: 2.30  Status: positive
Type III proteins may be localized at Golgi
Checking the amount of Basic Residues (nucleus)
Checking the 4 residue pattern for Nuclear Targeting
Checking the 7 residue pattern for Nuclear Targeting
Checking the Robbins & Dingwall consensus (nucleus)
Checking the RNA binding motif (nucleus or cytoplasm)
Nuclear Signal  Status: negative (0.00)
Check the Number of TMSs for typeIII (plasma memb.)
Checking N-myristoylation..
----- Final Results -----
endoplasmic reticulum (membrane) --- Certainty = 0.685(Affirmative) <succ>
plasma membrane --- Certainty = 0.640(Affirmative) <succ>
Golgi body --- Certainty = 0.460(Affirmative) <succ>
endoplasmic reticulum (lumen) --- Certainty = 0.100(Affirmative) <succ>
```

One predicted N-glycosylation sites of SEQ ID NO: 6046 is identified at residue 4:

Prediction of N-glycosylation Sites

| | | Jury | NGlyc | |
|---|---|---|---|---|
| Position | Potential | agreement | result | |
| 4 NGTI | 0.8430 | (9/9) | +++ | (SEQ ID NO: 7268) |

Accordingly, the invention comprises a polypeptide comprising a fragment of the amino acid sequence of SEQ ID NO: 6046, wherein said fragment comprises the N-glycosylation site identified above. The invention further comprises a polynucleotide encoding one or more of the polypeptides identified above.

The invention further comprises a polypeptide comprising a fragment of amino acid sequence SEQ ID NO: 6046, wherein said fragment does not include the N-glycosylation site identified above. The invention includes a polynucleotide encoding such a fragment.

A variant of SEQ ID NO: 6046 that is included within the invention is SEQ ID NO: 9963. Compared to SEQ ID NO: 6046, this sequence has Val at residue 72 instead of Ala.

T-epitopes for SEQ ID NO: 6046 are identified in Table 20. The invention includes a polypeptide for use as an antigen, wherein the polypeptide comprises: (a) an amino acid sequence selected from the group consisting of the T-epitope sequences identified in SEQ ID NOS: 8821-9018; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 8821-9018, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus. The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a polypeptide sequence comprising SEQ ID NO: 6047 or a fragment thereof or an amino acid sequence having sequence identity thereto. Predicted transmembrane regions of SEQ ID NO: 6047 are identified below.

| | | | |
|---|---|---|---|
| from | to | score | center |

Inside to outside helices: 2 found

| | | | |
|---|---|---|---|
| 7 (10) | 29 (27) | 729 | 17 |
| 21 (24) | 41 (41) | 640 | 34 |

Outside to inside helices: 2 found

| | | | |
|---|---|---|---|
| 4 (4) | 22 (19) | 874 | 12 |
| 22 (24) | 41 (41) | 499 | 31 |

Accordingly, the invention includes a polypeptide comprising a fragment of SEQ ID NO: 6047 wherein said fragment does not include one or more of the hydrophobic amino acid sequences identified above. Preferably, the fragment does not include the amino acids between positions selected from the group consisting of 4 to 22 and 22 to 41. The invention also includes a polynucleotide sequence encoding any of the above-identified polypeptides.

SEQ ID NO: 6047 is predicted to be a hypothetical protein of SARS virus. A prediction of the protein localization of SEQ ID NO: 6047 is set forth below. SEQ ID NO: 6047 is predicted to be located in one of the following locations: plasma membrane, endoplasmic reticulum, Golgi body, and microbody (peroxisome). SEQ ID NO: 6047 may be associated with an organelle inside an infected cell or with viral entry to a host cell.

Accordingly, SEQ ID NO: 6047 is a target for screening of chemical inhibitors to the SARS virus. The invention includes a polypeptide comprises SEQ ID NO: 6047 or a fragment thereof. The invention includes a polynucleotide encoding the polypeptide sequence of SEQ ID NO: 6047 or a fragment thereof. The invention includes a method of screening SEQ ID NO: 6047 for an inhibitor. The invention includes the recombinant expression of SEQ ID NO: 6047 in a host cell. The invention includes a small molecule which prevents the polypeptide of SEQ ID NO: 6047 from associating with an organelle inside of an infected cell or interacting with a host cell membrane. The invention includes a fusion protein wherein said fusion protein comprises SEQ ID NO: 6047. Predicted protein localization of SEQ ID NO: 6047 is set forth below.

```
PSORT --- Prediction of Protein Localization Sites
                                        version 6.4(WWW)
Species classification: 4
*** Reasoning Step: 1
Preliminary Calculation of ALOM (threshold: 0.5)
        count: 1
        Position of the most N-terminal TMS: 2 at i = 1
MTOP: membrane topology (Hartmann et al.)
        I(middle): 9  Charge diffirence(C − N): 0.5
McG: Examining signal sequence (McGeoch)
        Length of UR:  6
        Peak Value of UR:  3.08
        Net Charge of CR: 0
        Discriminant Score:  5.12
GvH: Examining signal sequence (von Heijne)
        Signal Score (−3.5): −4.45
        Possible cleavage site: 34
>>> Seems to have an uncleavable N-term signal seq.
Amino Acid Composition of Predicted Mature Form:
      calculated from 1
ALOM new cnt: 1 ** thrshld changed to −2
Cleavable signal was detected in ALOM?: 0B
ALOM: finding transmembrane regions (Klein et al.)
        count: 1  value: −2.44  threshold: −2.0
```

| | |
|---|---|
| INTEGRAL | Likelihood = −2.44 Transmembrane 2-18 (1-20) |
| PERIPHERAL | Likelihood = 1.22 |

```
        modified ALOM score:  0.59
>>> Seems to be a Type II (Ncyt Cexo) membrane protein
        The cytoplasmic tail is from 1 to 1 (1 Residues)
Rule: vesicular pathway
Rule: vesicular pathway
Rule: vesicular pathway
(5) or uncleavable?
Gavel: Examining the boundary of mitochondrial targeting seq.
          motif at: 5
        Uncleavable? Ipos set to: 15
Discrimination of mitochondrial target seq.:
        notclr (1.48)
Rule: vesicular pathway
Rule: vesicular pathway
Rule: vesicular pathway
*** Reasoning Step: 2
Relative position of the cytoplasmic tail: 1%
        Larger value (>30%) is favared for ER memb. proteins
Memb.protein with uncleavable signl is often at ER
KDEL  Count: 0
Checking apolar signal for intramitochondrial sorting
    (Gavel position 15)  from: 64  to: 93  Score: 30.0
>>> Seems to have an intramitochondrial signal
SKL motif (signal for peroxisomal protein):
        pos: −1(63), count: 0
Amino Acid Composition Tendency for Peroxisome: 1.91
Peroxisomal proteins? Status: notclr
        AAC score (peroxisome): 0.161
Amino Acid Composition tendency for lysosomal proteins
        score: 0.04 Status: notclr
Checking the consensus for Golgi
Checking the consensus for Golgi
Checking the cytoplasmic tail of type II (Golgi)
Checking the amount of Basic Residues (nucleus)
Checking the 4 residue pattern for Nuclear Targeting
Checking the 7 residue pattern for Nuclear Targeting
Checking the Robbins & Dingwall consensus (nucleus)
Checking the RNA binding motif (nucleus or cytoplasm)
Nuclear Signal  Status: negative (0.00)
Check mitochondrial signal for typeII (plasma memb.)
Type II is favored for plasma memb. proteins
Checking the NPXY motif..
Checking the YXRF motif..
Checking N-myristoylation..
----- Final Results -----
plasma membrane --- Certainty = 0.685(Affirmative) <succ>
endoplasmic reticulum (membrane) --- Certainty = 0.640(Affirmative) <succ>
Golgi body --- Certainty = 0.370(Affirmative) <succ>
microbody (peroxisome) --- Certainty = 0.161(Affirmative) <succ>
```

T-epitopes for SEQ ID NO: 6047 are identified in Table 21. The invention includes a polypeptide for use as an antigen, wherein the polypeptide comprises: (a) an amino acid sequence selected from the group consisting of the T-epitope sequences identified in SEQ ID NOS: 9019-9131; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 9019-9131, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus. The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a polypeptide comprising SEQ ID NO: 6048, a fragment thereof or an amino acid sequence having sequence identity thereto. Predicted transmembrane regions of SEQ ID NO: 6048 are identified below.

| | | | |
|---|---|---|---|
| from | to | score | center |

Inside to outside helices: 2 found

| | | | |
|---|---|---|---|
| 3 (3) | 18 (18) | 1857 | 10 |
| 100 (100) | 117 (115) | 2904 | 107 |

Outside to inside helices: 2 found

| | | | |
|---|---|---|---|
| 1 (1) | 15 (15) | 1299 | 8 |
| 100 (100) | 117 (115) | 3009 | 107 |

Accordingly, the invention includes a polypeptide comprising a fragment of SEQ ID NO: 6048 wherein said fragment does not include one or more of the hydrophobic amino acid sequences identified above. Preferably, the fragment does not include the amino acids between positions selected from the group consisting of 1 to 15 and 100 to 117. The invention also includes a polynucleotide sequence encoding any of the above-identified polypeptides.

SEQ ID NO: 6048 is predicted to be a hypothetical protein of SARS virus. A prediction of the protein localization of SEQ ID NO: 6048 is set forth below. SEQ ID NO: 6048 is predicted to be located in one of the following locations: plasma membrane, lysosome (membrane), microbody (peroxisome), and endoplasmic reticulum (membrane). SEQ ID NO: 6048 may be associated with an organelle inside an infected cell or may interact with a host cell plasma membrane during viral entry to the host cell.

Accordingly, SEQ ID NO: 6048 is a target for screening of chemical inhibitors to the SARS virus. The invention includes a polypeptide comprises SEQ ID NO: 6048 or a fragment thereof. The invention includes a polynucleotide encoding the polypeptide sequence of SEQ ID NO: 6048 or a fragment thereof. The invention includes a method of screening SEQ ID NO: 6048 for an inhibitor. The invention includes the recombinant expression of SEQ ID NO: 6048 in a host cell. The invention includes a small molecule which prevents the polypeptide of SEQ ID NO: 6048 from associating with an organelle inside of an infected cell or prevents the polypeptide from associating with the cell membrane of a host cell. The invention includes a fusion protein wherein said fusion protein comprises SEQ ID NO: 6048. Predicted protein localization of SEQ ID NO: 6048 is set forth below.

```
PSORT --- Prediction of Protein Localization Sites
                                        version 6.4(WWW)
Species classification: 4
*** Reasoning Step: 1
Preliminary Calculation of ALOM (threshold: 0.5)
        count: 2
        Position of the most N-terminal TMS: 3 at i = 2
MTOP: membrane topology (Hartmann et al.)
        I(middle): 10  Charge diffirence(C − N): −2.5
McG: Examining signal sequence (McGeoch)
        Length of UR:  13
        Peak Value of UR:  3.38
        Net Charge of CR: 1
        Discriminant Score:  10.02
GvH: Examining signal sequence (von Heijne)
        Signal Score (−3.5): 2.56
        Possible cleavage site: 15
>>> Seems to have a cleavable N-term signal seq.
Amino Acid Composition of Predicted Mature Form:
      calculated from 16
ALOM new cnt: 2 ** thrshld change to −2
Cleavable signal was detected in ALOM?: 1B
ALOM: finding transmembrane regions (Klein et al.)
        count: 1  value: −14.75  threshold: −2.0
```

| | |
|---|---|
| INTEGRAL | Likelihood = −14.75 Transmembrane 101-117 (95-120) |
| PERIPHERAL | Likelihood = 6.63 |

```
        modified ALOM score:  3.05
>>> Seems to be a Type Ia membrane protein
        The cytoplasmic tail is from 118 to 122 (5 Residues)
Rule: vesicular pathway
Rule: vesicular pathway
Rule: vesicular pathway
(15) or uncleavable?
Gavel: Examining the boundary of mitochondrial targeting seq.
          motif at: 15
        Uncleavable? Ipos set to: 25
Discrimination of mitochondrial target seq.:
        notclr (0.73)
Rule: vesicular pathway
Rule: vesicular pathway
Rule: vesicular pathway
*** Reasoning Step: 2
KDEL Count: 0
Checking apolar signal for intramitochondrial sorting
    (Gavel position 25)  from: 3  to: 12  Score: 8.5
SKL motif (signal for peroxisomal protein):
        pos: −1(122), count: 0
Amino Acid Composition Tendency for Peroxisome: 2.46
        AAC not from the N-term., score modified
Peroxisomal proteins? Status: notclr
        AAC score (peroxisome): 0.115
Amino Acid Composition tendency for lysosomal proteins
        score: −0.40  Status: negative
GY motif in the tail of typeIa? (lysosomal)
Checking the amount of Basic Residues (nucleus)
Checking the 4 residue pattern for Nuclear Targeting
Checking the 7 residue pattern for Nuclear Targeting
Checking the Robbins & Dingwall consensus (nucleus)
Checking the RNA binding motif (nucleus or cytoplasm)
Nuclear Signal  Status: negative (0.00)
Type Ia is favored for plasma memb. proteins
Checking the NPXY motif..
Checking the YXRF motif..
Checking N-myristoylation..
Checking GPI anchor..
>>> Seems to be GPI-anchored (0.85)
----- Final Results -----
plasma membrane --- Certainty = 0.919(Affirmative) <succ>
lysosome (membrane) --- Certainty = 0.200(Affirmative) <succ>
microbody (peroxisome) --- Certainty = 0.115(Affirmative) <succ>
endoplasmic reticulum (membrane) --- Certainty = 0.100(Affirmative) <succ>
```

T-epitopes for SEQ ID NO: 6048 are identified in Table 22. The invention includes a polypeptide for use as an antigen, wherein the polypeptide comprises: (a) an amino acid sequence selected from the group consisting of the T-epitope sequences identified in SEQ ID NOS: 9132-9308; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 9132-9308, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus. The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a polypeptide comprising SEQ ID NO: 6049, a fragment thereof or an amino acid sequence having sequence identity thereto. Predicted transmembrane or hydrophobic regions of SEQ ID NO: 6049 are identified below.

| | | | |
|---|---|---|---|
| from | to | score | center |

Inside to outside helices: 1 found

| | | | |
|---|---|---|---|
| 13 (13) | 30 (28) | 3532 | 20 |

Outside to inside helices: 1 found

| | | | |
|---|---|---|---|
| 9 (11) | 29 (26) | 3395 | 19 |

Accordingly, the invention includes a polypeptide comprising a fragment of SEQ ID NO: 6049 wherein said fragment does not include one or more of the hydrophobic amino acid sequences identified above. The invention also includes a polynucleotide sequence encoding any of the above-identified polypeptides.

SEQ ID NO: 6049 is predicted to be a hypothetical protein of SARS virus. A prediction of the protein localization of SEQ ID NO: 6049 is set forth below. SEQ ID NO: 6049 is predicted to be located in one of the following locations: outside, microbody (peroxisome), endoplasmic reticulum (membrane) and endoplasmic reticulum (lumen). The highest ranking indicates that SEQ ID NO: 6049 is located on the outside of a cell. Accordingly, SEQ ID NO: 6049 may be a surface exposed protein.

Accordingly, SEQ ID NO: 6049 may be used in an immunogenic composition to raise an immune response against the SARS virus. It also may be used to generate antibodies specific to the SARS virus. Such antibodies may be used in a method of treatment or prevention of a SARS virus infection. Such antibodies may further be used in a diagnostic test to identify the presence or absence of SARS virus in a biological sample.

The invention includes a polypeptide comprises SEQ ID NO: 6049 or a fragment thereof. The invention includes a polynucleotide encoding the polypeptide sequence of SEQ ID NO: 6049 or a fragment thereof. The invention includes a method of screening SEQ ID NO: 6049 for an inhibitor. The invention includes the recombinant expression of SEQ ID NO: 6049 in a host cell. The invention includes a fusion protein wherein said fusion protein comprises SEQ ID NO: 6049. Predicted protein localization of SEQ ID NO: 6049 is set forth below.

```
PSORT --- Prediction of Protein Localization Sites
                                        version 6.4(WWW)
Species classification: 4
*** Reasoning Step: 1
Preliminary Calculation of ALOM (threshold: 0.5)
        count: 1
        Position of the most N-terminal TMS: 11 at i = 1
MTOP: membrane topology (Hartmann et al.)
        I(middle): 18 Charge diffirence(C − N): −2.0
McG: Examining signal sequence (McGeoch)
        Length of UR:  24
        Peak Value of UR:  3.69
        Net Charge of CR: −2
        Discriminant Score:  13.56
GvH: Examining signal sequence (von Heijne)
        Signal Score (−3.5): 0.52
        Possible cleavage site: 25
>>> Seems to have a cleavable N-term signal seq.
Amino Acid Composition of Predicted Mature Form:
      calculated from 26
ALOM new cnt: 1 ** thrshld changed to −2
Cleavable signal was detected in ALOM?: 1B
ALOM: finding transmembrane regions (Klein et al.)
        count: 0  value: 14.80  threshold: −2.0
        PERIPHERAL  Likelihood = 14.80
        modified ALOM score: −3.86
Rule: vesicular pathway
Rule: vesicular pathway
Rule: vesicular pathway
(2) or uncleavable?
Gavel: Examining the boundary of mitochondrial targeting seq.
          motif at: 2
        Uncleavable? Ipos set to: 12
Discrimination of mitochondrial target seq.:
        notclr (1.42)
Rule: vesicular pathway
Rule: vesicular pathway
Rule: vesicular pathway
*** Reasoning Step: 2
KDEL  Count: 0
Number of Potential N-glycosylation Sites: 0
Out: score 0.800
Checking apolar signal for intramitochondrial sorting
    (Gavel position 12)  from: 44  to: 73  Score: 30.0
>>> Seems to have an intramitochondrial signal
SKL motif (signal for peroxisomal protein):
        pos: −1(44), count: 0
Amino Acid Composition Tendency for Peroxisome: 9.47
        AAC not from the N-term., score modified
Peroxisomal proteins? Status: notclr
        AAC score (peroxisome): 0.320
Amino Acid Composition tendency for lysosomal proteins
        score: −6.47  Status: negative
Number of NX(S/T) motif: 0
Checking the amount of Basic Residues (nucleus)
Checking the 4 residue pattern for Nuclear Targeting
Checking the 7 residue pattern for Nuclear Targeting
Checking the Robbins & Dingwall consensus (nucleus)
Checking the RNA binding motif (nucleus or cytoplasm)
Nuclear Signal  Status: negative (0.00)
Checking CaaX motif..
Checking N-myristoylation..
Checking CaaX motif..
----- Final Results -----
outside --- Certainty = 0.820(Affirmative) <succ>
microbody (peroxisome) --- Certainty = 0.320(Affirmative) <succ>
endoplasmic reticulum (membrane) --- Certainty = 0.100(Affirmative)
<succ>
endoplasmic reticulum (lumen) --- Certainty = 0.100(Affirmative) <succ>
```

T-epitopes for SEQ ID NO: 6049 are identified in Table 23. The invention includes a polypeptide for use as an antigen, wherein the polypeptide comprises: (a) an amino acid sequence selected from the group consisting of the T-epitope sequences identified in SEQ ID NOS: 9309-9437; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 9309-9437, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus. The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a polypeptide comprising SEQ ID NO: 6050 or a fragment thereof or an amino acid sequence having sequence identity thereto. Predicted transmembrane or hydrophobic regions are identified below.

| | | | |
|---|---|---|---|
| from | to | score | center |

Inside to outside helices: 1 found

| | | | |
|---|---|---|---|
| 13 (15) | 32 (30) | 558 | 23 |

Outside to inside helices: 1 found

| | | | |
|---|---|---|---|
| 16 (16) | 30 (30) | 364 | 23 |

Accordingly, the invention includes a polypeptide comprising a fragment of SEQ ID NO: 6050 wherein said fragment does not include one or more of the hydrophobic amino acid sequences identified above. The invention also includes a polynucleotide sequence encoding any of the above-identified polypeptides.

SEQ ID NO: 6050 is predicted to be a hypothetical protein of SARS virus. A prediction of the protein localization of SEQ ID NO: 6050 is set forth below. SEQ ID NO: 6050 is predicted to be located in one of the following locations: lysosome (lumen), mitochondrial matrix space, mitochondrial inner membrane, and mitochondrial intermembrane space. SEQ ID NO: 6050 may be associated with an organelle inside an infected cell during the viral replication cycle.

Accordingly, SEQ ID NO: 6050 is a target for screening of chemical inhibitors to the SARS virus. The invention includes a polypeptide comprises SEQ ID NO: 6050 or a fragment thereof. The invention includes a polynucleotide encoding the polypeptide sequence of SEQ ID NO: 6050 or a fragment thereof. The invention includes a method of screening SEQ ID NO: 6050 for an inhibitor. The invention includes the recombinant expression of SEQ ID NO: 6050 in a host cell. The invention includes a small molecule which prevents the polypeptide of SEQ ID NO: 6050 from associating with an organelle inside of an infected cell or prevents the polypeptide from associating with the cell membrane of a host cell. The invention includes a fusion protein wherein said fusion protein comprises SEQ ID NO: 6050. Predicted protein localization of SEQ ID NO: 6050 is set forth below.

PSORT --- Prediction of Protein Localization Sites

version 6.4(WWW)

```
MYSEQ 84 Residues
Species classification: 4
*** Reasoning Step: 1
Preliminary Calculation of ALOM (threshold: 0.5)
  count: 0
McG: Examining signal sequence (McGeoch)
  Length of UR: 3
  Peak Value of UR: 1.46
  Net Charge of CR: 2
  Discriminant Score: −5.73
GvH: Examining signal sequence (von Heijne)
  Signal Score (−3.5): −0.12
  Possible cleavage site: 29
>>> Seems to have no N-terminal signal seq.
Amino Acid Composition of Predicted Mature Form:
  calculated from 1
ALOM new cnt: 0 ** thrshld changed to −2
Cleavable signal was detected in ALOM?: 0B
ALOM: finding transmembrane regions (Klein et al.)
  count: 0 value: 8.43 threshold: −2.0
  PERIPHERAL Likelihood = 8.43
  modified ALOM score: −2.59
Gavel: Examining the boundary of mitochondrial targeting seq.
    motif at: 61
  ARCWYL
Discrimination of mitochondrial target seq.:
  positive (1.66)
Rule: mitochondrial protein
Rule: mitochondrial protein
Rule: mitochondrial protein
Rule: mitochondrial protein
*** Reasoning Step: 2
KDEL Count: 0
Checking apolar signal for intramitochondrial sorting
  (Gavel position 61) from: 52 to: 58 Score: 6.0
Mitochondrial matrix? Score: 0.38
SKL motif (signal for peroxisomal protein):
  pos: −1(84), count: 0
Amino Acid Composition Tendency for Peroxisome: 1.47
Peroxisomal proteins? Status: notclr
  AAC score (peroxisome): 0.263
Amino Acid Composition tendency for lysosomal proteins
  score: 2.86 Status: positive
Modified score for lysosome: 0.850
Checking the amount of Basic Residues (nucleus)
Checking the 4 residue pattern for Nuclear Targeting
Checking the 7 residue pattern for Nuclear Targeting
Checking the Robbins & Dingwall consensus (nucleus)
Checking the RNA binding motif (nucleus or cytoplasm)
Nuclear Signal Status: negative (0.00)
Checking CaaX motif..
Checking N-myristoylation..
Checking CaaX motif..
----- Final Results -----
lysosome (lumen) --- Certainty = 0.850(Affirmative) < succ>
mitochondrial matrix space --- Certainty = 0.544(Affirmative) < succ>
mitochondrial inner membrane --- Certainty = 0.266(Affirmative) < succ>
mitochondrial intermembrane space --- Certainty = 0.266(Affirmative)
< succ>
```

One predicted N-glycosylation sites of SEQ ID NO: 6050 is identified at residue 43:

| | | Jury | NGlyc | |
|---|---|---|---|---|
| Position | Potential | agreement | result | |
| 43 NVTI | 0.6713 | (9/9) | ++ | (SEQ ID NO: 7269) |

Accordingly, the invention comprises a polypeptide comprising a fragment of the amino acid sequence of SEQ ID NO: 6050 wherein said fragment comprises the N-glycosylation site identified above. The invention further comprises a polynucleotide encoding one or more of the polypeptides identified above.

The invention further comprises a polypeptide comprising a fragment of amino acid sequence SEQ ID NO: 6050 wherein said fragment does not include the N-glycosylation site identified above. The invention includes a polynucleotide encoding such a fragment.

T-epitopes for SEQ ID NO: 6050 are identified in Table 24. The invention includes a polypeptide for use as an antigen, wherein the polypeptide comprises: (a) an amino acid sequence selected from the group consisting of the T-epitope sequences identified in SEQ ID NOS: 9438-9538; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 9438-9538, or a polynucleotide encoding such a polypeptide.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus. The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a polypeptide sequence comprising SEQ ID NO: 6051 or a fragment thereof or an amino acid sequence having sequence identity thereto. The invention includes a polypeptide sequence comprising SEQ ID NO: 6052 or a fragment thereof or an amino acid sequence having sequence identity thereto.

SEQ ID NO: 6051 and SEQ ID NO: 6052 demonstrate functional homology with a nucleocapsid protein of a coronavirus. The invention includes a diagnostic kit comprising a polypeptide comprising SEQ ID NO: 6051, SEQ ID NO: 6052 or a fragment thereof. The invention includes a diagnostic kit comprising a polynucleotide encoding SEQ ID NO: 6051, SEQ ID NO: 6052 or a fragment thereof. The invention includes an immunogenic composition comprising SEQ ID NO: 6051, SEQ ID NO: 6052 or a fragment thereof. The invention includes an antibody which recognizes a polypeptide comprising SEQ ID NO: 6051, SEQ ID NO: 6052 or a fragment thereof.

SEQ ID NO: 6051 is predicted to be phosphorylated at Ser-79; Thr-92; Ser-106; Thr-116; Thr-142; Ser-184; Ser-188; Ser-202; Ser-236; Thr-248; Ser-251; Ser-256; Thr-377. Accordingly, the invention includes a polypeptide comprising a fragment of SEQ ID NO: 6051 wherein said fragment includes one or more of the amino acid residues of SEQ ID NO: 6051 selected from the group consisting of Ser-79; Thr-92; Ser-106; Thr-116; Thr-142; Ser-184; Ser-188; Ser-202; Ser-236; Thr-248; Ser-251; Ser-256; Thr-377. The invention further includes a polypeptide comprising a fragment of SEQ ID NO: 6051 wherein said fragment does not include one or more of the amino acid residues of SEQ ID NO: 6051 selected from the group consisting of Ser-79; Thr-92; Ser-106; Thr-116; Thr-142; Ser-184; Ser-188; Ser-202; Ser-236; Thr-248; Ser-251; Ser-256; Thr-377. Two further useful fragments of the N protein (e.g. for immunoassay) are SEQ ID NOS: 9783 & 9784, which are lysine-rich and can be used to distinguish the SARS virus from other coronaviruses.

Predicted transmembrane regions of SEQ ID NO: 6051 are identified below.

| | | | |
|---|---|---|---|
| from | to | score | center |

Inside to outside helices: 1 found

| | | | |
|---|---|---|---|
| 304 (304) | 323 (319) | 495 | 312 |

Outside to inside helices: 2 found

| | | | |
|---|---|---|---|
| 304 (304) | 319 (319) | 597 | 312 |

Accordingly, the invention includes a polypeptide comprising a fragment of SEQ ID NO: 6051 wherein said fragment does not include one or more of the hydrophobic amino acid sequences identified above. The invention also includes a polynucleotide sequence encoding any of the above-identified polypeptides.

Predicted protein localization of SEQ ID NO: 6051 is set forth below. SEQ ID NO: 6051 is predicted to be localized near the nucleus, lysosome (lumen), mitochondrial matrix space, and microbody (peroxisome). The highest ranking is for localization near the nucleus. Coronavirus nucleocapsid proteins are known to bind to viral RNA. Coronavirus nucleocapsid proteins are also thought to be important for cell mediated immunity. Accordingly, the invention includes a polynucleotide comprising SEQ ID NO: 6051. The invention further includes a viral vector or particle suitable for in vivo delivery of the polynucleotide sequence comprising a SARS virus nucleocapsid polynucleotide sequence or a fragment thereof. In one embodiment, the polynucleotide comprises SEQ ID NO: 6051 or a fragment thereof. The invention further includes a method for eliciting a cell mediated immune response comprising delivering a polynucleotide encoding a SARS virus nucleocapsid protein or a fragment thereof to a mammal. In one embodiment, the polynucleotide comprising SEQ ID NO: 6051 or a fragment thereof.

The invention further includes a method of screening SEQ ID NO: 6051 for an inhibitor. The invention includes the recombinant expression of SEQ ID NO: 6051 in a host cell. The invention includes a small molecule which prevents the polypeptide of SEQ ID NO: 6051 from binding to SARS virus RNA during viral replication. The invention includes a fusion protein wherein said fusion protein comprises SEQ ID NO: 6051. Predicted protein localization of SEQ ID NO: 6051 is set forth below.

PSORT --- Prediction of Protein Localization Sites

version 6.4(WWW)

```
Species classification: 4
*** Reasoning Step: 1
Preliminary Calculation of ALOM (threshold: 0.5)
  count: 0
McG: Examining signal sequence (McGeoch)
  Length of UR: 3
  Peak Value of UR: 0.19
  Net Charge of CR: 0
  Discriminant Score: −15.98
GvH: Examining signal sequence (von Heijne)
  Signal Score (−3.5): −6.36
  Possible cleavage site: 58
>>> Seems to have no N-terminal signal seq.
Amino Acid Composition of Predicted Mature Form:
  calculated from 1
ALOM new cnt: 0 ** thrshld changed to −2
Cleavable signal was detected in ALOM?: 0B
ALOM: finding transmembrane regions (Klein et al.)
  count: 0 value: 5.04 threshold: −2.0
  PERIPHERAL Likelihood = 5.04
  modified ALOM score: −1.91
Gavel: Examining the boundary of mitochondrial targeting seq.
  motif at: 17
  PRITFG
Discrimination of mitochondrial target seq.:
  negative (−3.97)
*** Reasoning Step: 2
KDEL Count: 0
Checking apolar signal for intramitochondrial sorting
Mitochondrial matrix? Score: 0.10
SKL motif (signal for peroxisomal protein):
  pos: −1(399), count: 0
Amino Acid Composition Tendency for Peroxisome: 0.04
Peroxisomal proteins? Status: notclr
  AAC score (peroxisome): 0.072
Amino Acid Composition tendency for lysosomal proteins
  score: 0.96 Status: notclr
Modified score for lysosome: 0.246
Checking the amount of Basic Residues (nucleus)
Checking the 4 residue pattern for Nuclear Targeting
  Found: pos: 256 (4) KKPR
  Found: pos: 372 (5) KKKK
Checking the 7 residue pattern for Nuclear Targeting
Checking the Robbins & Dingwall consensus (nucleus)
  Found: pos: 372 (3) KK KKTDEAQPLP QRQKK
Found: pos: 373 (3) KK KTDEAQPLPQ RQKKQ
Final Robbins Score (nucleus): 0.80
Checking the RNA binding motif (nucleus or cytoplasm)
nuc modified. Score: 0.90
Nuclear Signal Status: positive (0.90)
Checking CaaX motif..
Checking N-myristoylation..
Checking CaaX motif..
----- Final Results -----
nucleus --- Certainty = 0.980(Affirmative) < succ>
lysosome (lumen) --- Certainty = 0.246(Affirmative) < succ>
mitochondrial matrix space --- Certainty = 0.100(Affirmative) < succ>
microbody (peroxisome) --- Certainty = 0.072(Affirmative) < succ>
```

Predicted N-glycosylation sites of SEQ ID NO: 6051 are identified below.

| | | Jury | NGlyc | |
|---|---|---|---|---|
| Position | Potential | agreement | result | |
| 48 NNTA | 0.6879 | (9/9) | ++ | (SEQ ID NO: 7270) |
| 270 NVTQ | 0.7684 | (9/9) | +++ | (SEQ ID NO: 7271) |

| Residue No. | | Potential | Threshold | Assignment |
|---|---|---|---|---|
| Thr | 166 | 0.8547 | 0.6439 | T |
| Thr | 367 | 0.5575 | 0.5403 | T |
| Thr | 394 | 0.8217 | 0.5821 | T |

Accordingly, the invention comprises a polypeptide comprising a fragment of the amino acid sequence of SEQ ID NO: 6051 wherein said fragment comprises one or more of the N-glycosylation sites identified above. The invention further comprises a polynucleotide encoding one or more of the polypeptides identified above.

The invention further comprises a polypeptide comprising a fragment of amino acid sequence SEQ ID NO: 6051 wherein said fragment does not include one or more of the N-glycosylation sites identified above. The invention includes a polynucleotide encoding such a fragment.

T-epitopes for SEQ ID NO: 6052 are identified in Table 25. The invention includes a polypeptide for use as an antigen, wherein the polypeptide comprises: (a) an amino acid sequence selected from the group consisting of the T-epitope sequences identified in SEQ ID NOS: 9539-9752; (b) an amino acid sequence having sequence identity to an amino acid sequence of (a). The invention further comprising a polynucleotide sequence encoding the polypeptides of (a) or (b). The invention further comprising a method of expression or delivery of such polynucleotides through viral vectors and/or viral particles. The invention further comprises a polypeptide comprising two or more of the T-epitope sequences identified in SEQ ID NOS: 9539-9752, or a polynucleotide encoding such a polypeptide.

A variant of SEQ ID NO: 6052 that is included within the invention is SEQ ID NO: 9964. Compared to SEQ ID NO: 6052, this sequence has Ile at residue 54 instead of Thr.

The use as an antigen is preferably a use: (1) as a T-cell antigen; (2) for generating a complex between a class I MHC protein (e.g. a class I HLA) and a fragment of said antigen; (3) as an antigen for raising a cell-mediated immune response; and/or (4) as an antigen for raising a CTL response. The use preferably protects or treats disease and/or infection caused by a SARS virus. The invention provides the use of a polypeptide in the manufacture of a medicament for immunising a mammal (typically a human) against SARS viral infection wherein the polypeptide is as defined above.

The invention provides a method of raising an immune response in a mammal (typically a human), comprising the step of administering to the mammal a polypeptide as defined above, wherein said immune response is a cell-mediated immune response and, preferably, a CTL response. The immune response is preferably protective or therapeutic.

The invention includes a composition comprising a SARS virus nucleocapsid protein or a fragment thereof and further comprising a SARS virus membrane protein or a fragment thereof. The composition may further comprising one or more adjuvants discussed below.

The invention further includes a composition comprising a polypeptide comprising SEQ ID NO: 6051 or a fragment thereof or a sequence having sequence identity thereto and further comprising a polypeptide comprising SEQ ID NO: 6040, or a fragment thereof or a sequence having sequence identity thereto. Such composition may be used, for instance, in a vaccine. Such composition may further comprise one or more adjuvants discussed below.

The invention includes a composition comprising a SARS virus nucleocapsid protein or a fragment thereof and a SARS virus spike protein or a fragment thereof. In one embodiment the nucleocapsid protein comprises a polypeptide sequence comprising SEQ ID NO: 6051 or a fragment thereof or a sequence having sequence identity thereto. In one embodiment, the spike protein comprises a polynucleotide comprising SEQ ID NO: 6042 or a fragment thereof or a sequence having sequence identity thereto. The composition may further comprise one or more of the adjuvants discussed below.

The invention further includes a composition comprising antibodies specific to a SARS virus nucleocapsid protein and comprising antibodies specific to a SARS virus spike protein. In one embodiment the antibody is specific to a nucleocapsid protein comprises a polypeptide sequence comprising SEQ ID NO: 6051 or a fragment thereof or a sequence having sequence identity thereto. In one embodiment, the antibody is is specific to a spike protein comprises a polynucleotide comprising SEQ ID NO: 6042 or a fragment thereof or a sequence having sequence identity thereto.

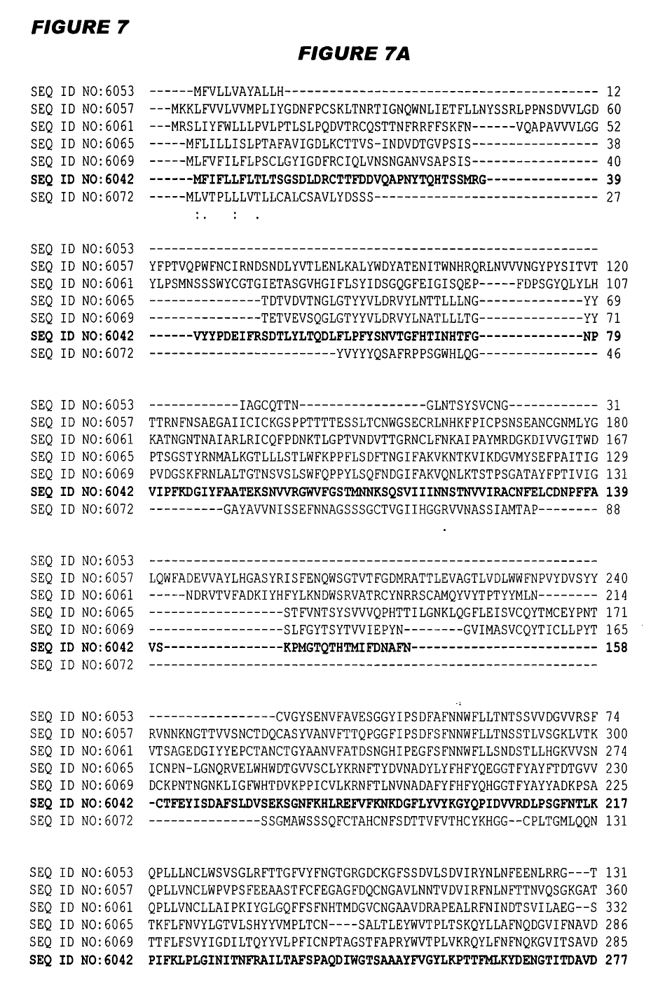

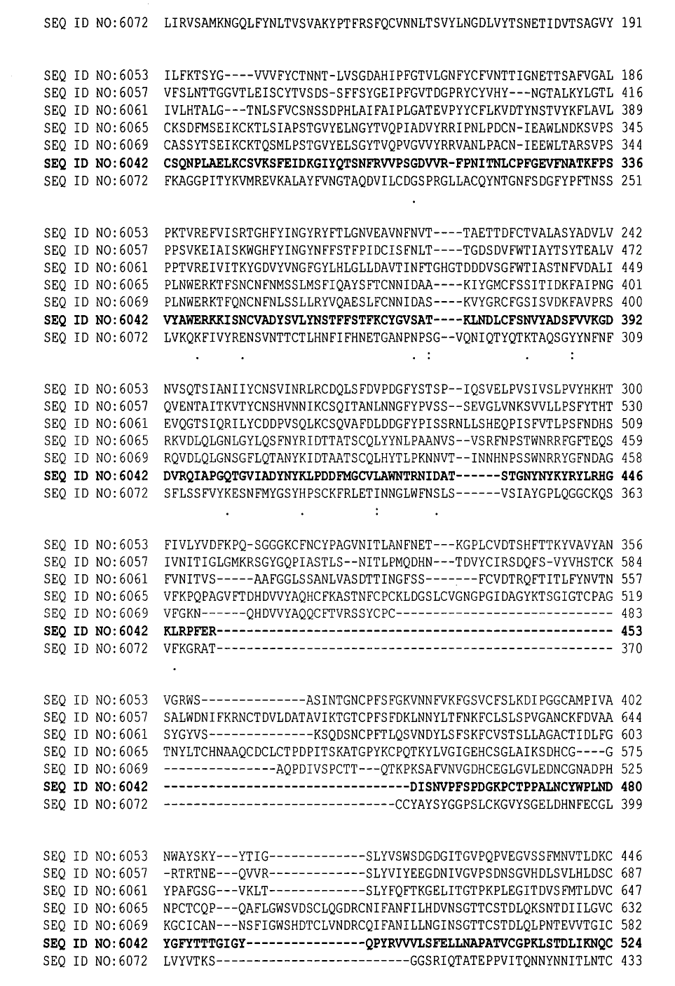

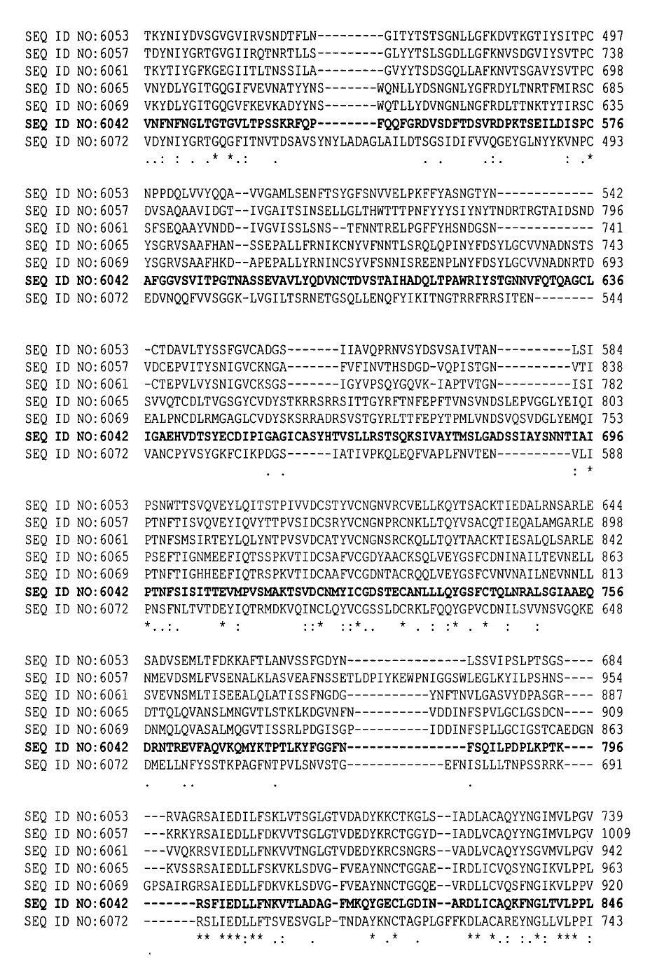

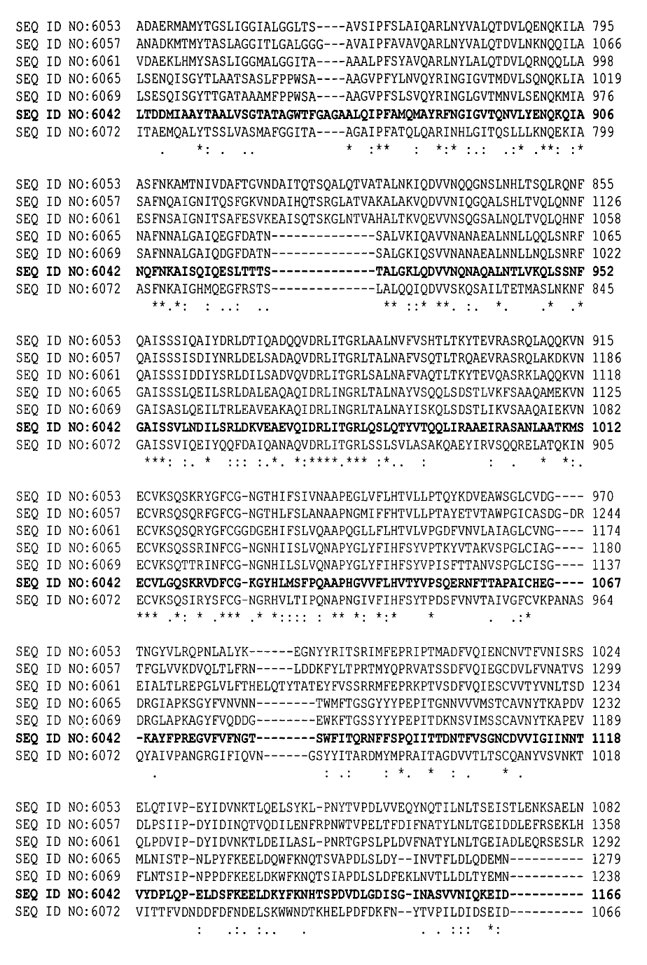

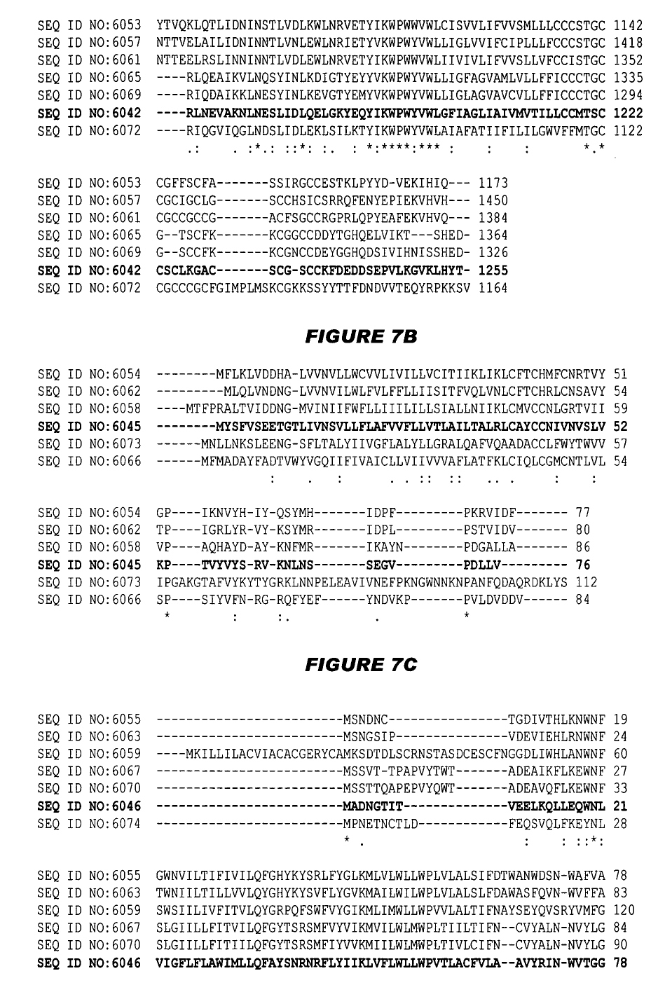

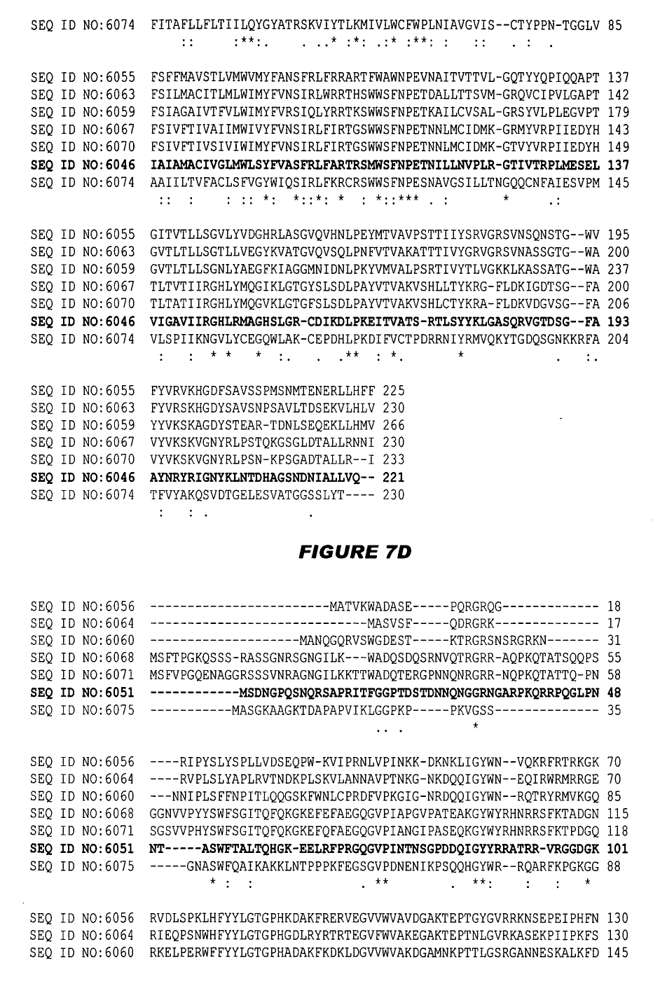

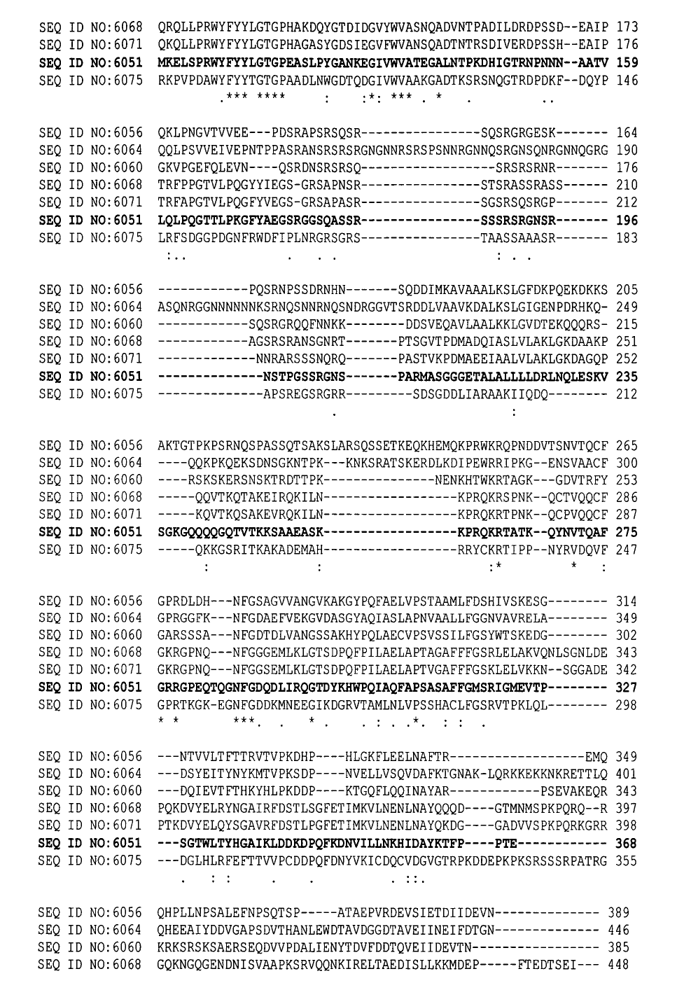

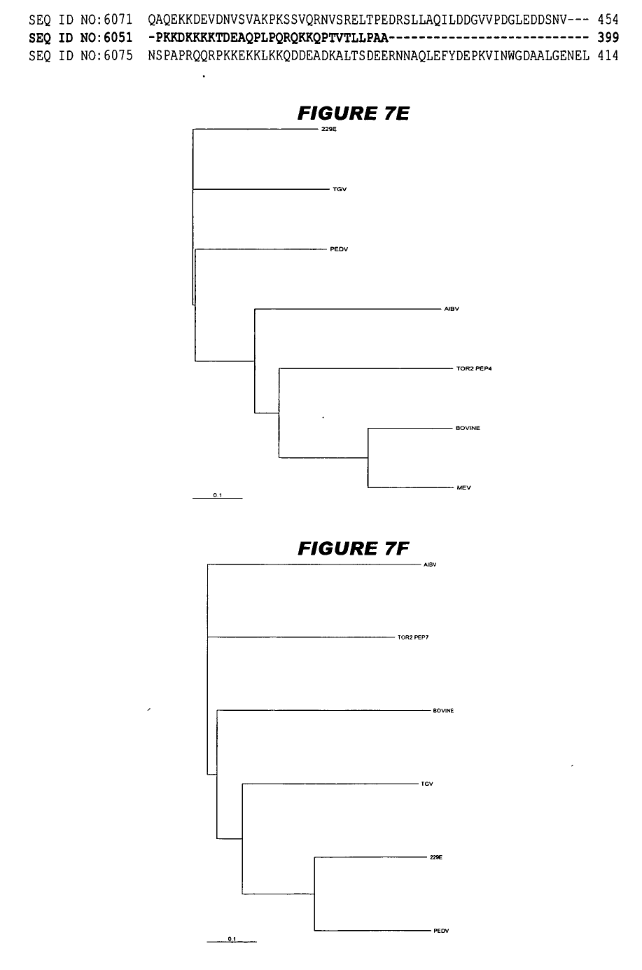

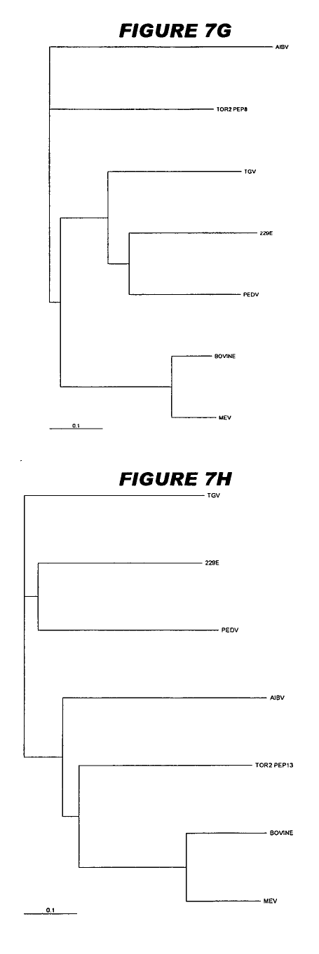

The invention further includes polynucleotide sequences, and fragments thereof, of a SARS virus which are conserved among coronaviruses, and polypeptides encoded thereby. Such conserved sequences can be identified in the alignments shown in FIG. 7. Such conserved sequences may be used in the vaccines of the invention or in the diagnostic reagents, kits and methods of the invention.

The invention further includes polynucleotide sequences, and fragments thereof, of a SARS virus which are specific to SARS virus and not shared with coronaviruses. Such SARS specific sequences are also identified as SEQ ID NOS: 6040, 6043, 6044, 6047, 6048, 6049 and 6050. Such SARS specific sequences may be used in the vaccines of the invention or in the diagnostic reagents, kits and methods of the invention.

The invention also includes polynucleotide sequences which can be used as probes or primers for diagnostic reagents, kits (comprising such reagents) and methods which can be used to diagnose or identify the presence or absence of a SARS virus in a biological sample. The invention includes a polynucleotide sequence comprising one or more of the primer sequences identified in SEQ ID NOS: 6076-6265 (Table 5). The invention further includes polynucleotide sequence comprising the complement of one or more of the primer sequences identified in SEQ ID NOS: 6076-6265.

The invention also includes polynucleotide sequences which can be used as probes or primers for diagnostic reagents, kits (comprising such reagents) and methods which can be used to diagnose or identify the presence or absence of a SARS virus in a biological sample. The invention includes a polynucleotide sequence comprising one or more of the primer sequences identified in SEQ ID NOS: 6266-6343 (Table 6). The invention further includes polynucleotide sequence comprising the complement of one or more of the primer sequences identified in SEQ ID NOS: 6266-6343.

The invention also includes polynucleotide sequences which can be used as probes or primers for diagnostic reagents, kits (comprising such reagents) and methods which can be used to diagnose or identify the presence or absence of a SARS virus in a biological sample. The invention includes a polynucleotide sequence comprising one or more of the primer sequences identified in SEQ ID NOS: 6344-6392 (Table 7). The invention further includes polynucleotide sequence comprising the complement of one or more of the primer sequences identified in SEQ ID NOS: 6344-6392.

The invention also includes polynucleotide sequences which can be used as probes or primers for diagnostic reagents, kits (comprising such reagents) and methods which can be used to diagnose or identify the presence or absence of a SARS virus in a biological sample. The invention includes a polynucleotide sequence comprising one or more of the primer sequences identified in SEQ ID NOS: 6393-6559 (Tables 8 & 9). The invention further includes polynucleotide sequence comprising the complement of one or more of the primer sequences identified in SEQ ID NOS: 6393-6559.

The invention also includes polynucleotide sequences which can be used as probes or primers for diagnostic reagents, kits (comprising such reagents) and methods which can be used to diagnose or identify the presence or absence of a SARS virus in a biological sample. The invention includes a polynucleotide sequence comprising one or more of the primer and probe sequences identified in SEQ ID NOS: 6560-6568. The invention further includes polynucleotide sequence comprising the complement of one or more of the primer sequences identified in SEQ ID NOS: 6560-6568.

The invention includes a polypeptide sequence comprising any one of even-numbered SEQ ID NOS: 7272-7290, or a fragment thereof, or a sequence having sequence identity thereto. The invention further includes a polynucleotide sequence encoding any one of even-numbered SEQ ID NOS: 7272-7290, or a fragment thereof, or a sequence having sequence identity thereto. Examples of such polynucleotide sequences are odd-numbered SEQ ID NOS: 7273-7291.

The invention includes a polynucleotide sequence comprising an intergenic sequence which is common to each open reading frame of the SARS virus. The SARS virus is thought to use this sequence to signal translation of the open reading frame. The intergenic sequence comprises a 10 mer SEQ ID NO: 7292, or optionally a hexamer SEQ ID NO: 7293. When the virus transcribes its positive (+) RNA strand to (−) RNA strand, the virus replicating structure uses the (−) strand template to transcribe nucleotides at the 5′ end prior to the first intergenic sequence, followed by the intergenic sequence, followed by the selected open reading frame. The virus then creates multiple mRNAs comprising the 5′ end, the intergenic sequence and coding sequence. For more details on Nidovriales replication (including Coronavirus) see e.g., Ziebuhr et al., “Virus-encoded proteinases and proteolytic processing in the Nidovirales”, Journal of General Virology 81:853-879 (2000), incorporated herein by reference in its entirety.

The invention comprising a polynucleotide sequence comprising SEQ ID NO: 7292 or the complement thereof. The invention comprising a polynucleotide sequence comprising SEQ ID NO: 7293 or the complement thereof. The invention further comprises a polynucleotide sequence comprising nucleotides from the 5′ end of the SARS viral genome, or its reverse complement, and farther comprising an intergenic sequence or its reverse complement. The polynucleotide may further comprise one or more of the SARS virus open reading frames. Examples of polynucleotide sequences comprising nucleotides from the 5′ end of the SARS virus genome followed by the intergenic sequence are SEQ ID NOS: 7294-7301.

The invention includes a polynucleotide sequence comprising a sequence selected from the group consisting of SEQ ID NO: 7292, SEQ ID NO: 7293, SEQ ID NO: 7294, SEQ ID NO: 7295, SEQ ID NO: 7296, SEQ ID NO: 7297, SEQ ID NO: 7298, SEQ ID NO: 7299, SEQ ID NO: 7300 and SEQ ID NO: 7301, or a fragment thereof, or a sequence having sequence identity thereto. In one embodiment, the polynucleotide does not consist entirely of a known SARS virus sequence.

The SARS virus intergenic sequence can be used to create a RNAi molecule. Such a SARS virus specific RNAi molecule can be used to treat SARS virus infection. The invention includes a RNAi molecule comprising a double stranded RNA molecule wherein one RNA strand comprises a sequence selected from the group consisting of SEQ ID NO: 7292, SEQ ID NO: 7293, SEQ ID NO: 7294, SEQ ID NO: 7295, SEQ ID NO: 7296, SEQ ID NO: 7297, SEQ ID NO: 7298, SEQ ID NO: 7299, SEQ ID NO: 7300 and SEQ ID NO: 7301, or a fragment thereof. Preferably, said RNA strand comprises a sequence selected from the group consisting of SEQ ID NO: 7292 and SEQ ID NO: 7293. Preferably, the other RNA strand comprises the reverse complement of the first strand or a polynucleotide sequence which hybridizes to the first strand.

The invention includes the use of RNAi in a method of treatment for SARS virus infection comprising administering to a mammal an effective amount of the si RNA molecule. Preferably, the RNAi molecule comprises the molecule described above. Further discussion of the RNAi applications of the intergenic sequence is included in section IV of the specification below.

The invention also includes the use of a SARS virus antisense nucleotide sequence, preferably antisense directed to the SARS virus intergenic sequence. Such an antisense sequence may be used in the treatment of a subject infected with the SARS virus. The antisense of the SARS virus intergenic sequence can be designed to bind to the SARS viral polynucleotides to block access of the viral replication machinery to the intergenic sequence. Such an antisense sequence may also be used to identify the presence or absence of a SARS virus in a biological sample. The antisence can itself be labeled or the antisense associated with viral polynucleotides can be detected by means known in the art.

Antisense nucleic acids are designed to specifically bind to RNA, resulting in the formation of RNA-DNA or RNA-RNA hybrids, with an arrest of DNA replication, reverse transcription or messenger RNA translation. Antisense polynucleotides based on a selected sequence can interfere with expression of the corresponding gene. Antisense polynucleotides will bind and/or interfere with the translation of the corresponding mRNA.

The invention also includes the use of the intergenic region with a ribozyme.

Trans-cleaving catalytic RNAs (ribozymes) are RNA molecules possessing endoribonuclease activity. Ribozymes are specifically designed for a particular target, and the target message must contain a specific nucleotide sequence. They are engineered to cleave any RNA species site-specifically in the background of cellular RNA. The cleavage event renders the mRNA unstable and prevents protein expression. Importantly, ribozymes can be used to inhibit expression of a gene of unknown function for the purpose of determining its function in an in vitro or in vivo context, by detecting the phenotypic effect.

One commonly used ribozyme motif is the hammerhead, for which the substrate sequence requirements are minimal. Design of the hammerhead ribozyme is disclosed in Usman et al., Current Opin. Struct. Biol. (1996) 6:527-533. Usman also discusses the therapeutic uses of ribozymes. Ribozymes can also be prepared and used as described in Long et al., FASEB J. (1993) 7:25; Symons, Ann. Rev. Biochem. (1992) 61:641; Perrotta et al., Biochem. (1992) 31:16-17; Ojwang et al., Proc. Natl. Acad. Sci. (USA) (1992) 89:10802-10806; and U.S. Pat. No. 5,254,678. Ribozyme cleavage of HIV-I RNA is described in U.S. Pat. No. 5,144,019; methods of cleaving RNA using ribozymes is described in U.S. Pat. No. 5,116,742; and methods for increasing the specificity of ribozymes are described in U.S. Pat. No. 5,225,337 and Koizumi et al., Nucleic Acid Res. (1989) 17:7059-7071. Preparation and use of ribozyme fragments in a hammerhead structure are also described by Koizumi et al., Nucleic Acids Res. (1989) 17:7059-7071. Preparation and use of ribozyme fragments in a hairpin structure are described by Chowrira & Burke, Nucleic Acids Res. (1992) 20:2835. Ribozymes can also be made by rolling transcription as described in Daubendiek & Kool, Nat. Biotechnol. (1997) 15(3):273-277.

The hybridizing region of the ribozyme may be modified or may be prepared as a branched structure as described in Horn & Urdea, Nucleic Acids Res. (1989) 17:6959-67. The basic structure of the ribozymes may also be chemically altered in ways familiar to those skilled in the art, and chemically synthesized ribozymes can be administered as synthetic oligonucleotide derivatives modified by monomeric units. In a therapeutic context, liposome mediated delivery of ribozymes improves cellular uptake, as described in Birikh et al., Eur. J. Biochem. (1997) 245:1-16.

Therapeutic and functional genomic applications of ribozymes proceed beginning with knowledge of a portion of the coding sequence of the gene to be inhibited. In the present invention, the target sequence preferably comprises the intergeneic sequence of the SARS virus. Preferably, the sequence is selected from the group consisting of SEQ ID NO: 7292 and SEQ ID NO: 7293. A target cleavage site is selected in the target sequence, and a ribozyme is constructed based on the 5′ and 3′ nucleotide sequences that flank the cleavage site. Preferably, the 5′ nucleotide sequence includes the 5′ untranslated region of the SARS virus. The ribozyme may then further be constructed from one or more of the polynucleotide sequences selected from the group consisting of SEQ ID NO: 7294, SEQ ID NO: 7295, SEQ ID NO: 7296, SEQ ID NO: 7297, SEQ ID NO: 7298, SEQ ID NO: 7299, SEQ ID NO: 7300 and SEQ ID NO: 7301.

Antisense treatment of HIV infection is described in the following references, each of which is incorporated herein by reference in their entirety. (antisense RNA complementary to the mRNA of gag, tat, rev, env) (Sezakiel et al., 1991, J. Virol. 65:468-472; Chatterjee et al., 1992, Science 258:1485-1488; Rhodes et al., 1990, J. Gen. Virol. 71:1965. Rhodes et al., 1991, AIDS 5:145-151; Sezakiel et al., 1992, J. Virol. 66:5576-5581; Joshi et al., 1991, J. Virol. 65:5524-5530).

The invention includes the use of decoy RNA to disrupt the SARS virus replication and life cycle. Methods of making and using such decoy RNA for treatment of a viral infection are known in the art. The invention includes delivery of genes encoding, for example, the SARS virus intergenic sequence, to infected cells. Preferably, the sequence comprises one or more of the sequences selected from the group consisting of SEQ ID NO: 7292, SEQ ID NO: 7293, SEQ ID NO: 7294, SEQ ID NO: 7295, SEQ ID NO: 7296, SEQ ID NO: 7297, SEQ ID NO: 7298, SEQ ID NO: 7299, SEQ ID NO: 7300 and SEQ ID NO: 7301. Preferably, the sequence comprises one or more of the sequences selected from the group consisting of SEQ ID NO: 7292 and SEQ ID NO: 7293. Preferably, the sequence comprises SEQ ID NO: 7293.

In the present invention, delivery of intergenic sequence which is not linked to the SARS virus open reading frames disrupts the translation process of the viral RNA and decreases the production of vial proteins. Similar methods of treatment for HIV viral infection have been described. The following references discuss the use of decoy RNA of HIV TAR or RRE for treatment of HIV infection. Each of these references is incorporated herein by reference in their entirety. (Sullenger et al., 1990, Cell 63:601-608; Sullenger et al., 1991, J. Virol. 65:6811-6816; Lisziewicz et al., 1993, New Biol. 3:82-89; Lee et al., 1994, J. Virol. 68:8254-8264), ribozymes (Sarver et al., 1990, Science 247:1222-1225; Wecrasinghe et al., 1991, J. Virol. 65:5531-5534; Dropulic et al., 1992, J. Virol. 66:1432-1441; Ojwang et al., 1992, Proc. Natl. Acad. Sci. USA. 89:10802-10806; Yu et al., 1993, Proc. Natl. Acad. Sci. USA. 90:6340-6344; Yu et al., 1995, Proc. Natl. Acad. Sci. USA. 92:699-703; Yamada et al., 1994, Gene Therapy 1:38-45).

The invention includes the use of the SARS virus intergenic sequence in diagnostic reagents, kits (comprising such reagents) and methods which can be used to diagnose or identify the presence or absence of a SARS virus in a biological sample. Such diagnostic reagents, kits, and methods are further discussed in Section II of the specification.

The invention includes a pair of primers for amplifying a SARS polynucleotide sequence comprising (i) a first primer comprising a sequence which is substantially identical to a portion of a sequence selected from the group consisting of SEQ ID NO: 7292, SEQ ID NO: 7293, SEQ ID NO: 7294, SEQ ID NO: 7295, SEQ ID NO: 7296, SEQ ID NO: 7297, SEQ ID NO: 7298, SEQ ID NO: 7299, SEQ ID NO: 7300 and SEQ ID NO: 7301 and (ii) a second primer comprising a sequence which is substantially complementary to a portion of a sequence selected from the group consisting of the sequence SEQ ID NO: 1 and the sequence SEQ ID NO: 2, such that the primer pair (i) and (ii) defines a template sequence within a sequence from the group consisting of the sequence SEQ ID NO: 1 and the sequence SEQ ID NO: 2. Preferably, the (i) first primer comprises a sequence which is substantially identical to a portion of a sequence selected from the group consisting of SEQ ID NO: 7292 and SEQ ID NO: 7293. Preferably, the (i) first primer comprises a sequence which is substantially identical to a portion of the sequence of SEQ ID NO: 7293. The amplicon defined by said first and second primers is preferably between 50 and 250 nucleotides in length. The primers may optionally be labeled to facilitate their detection. Methods and compositions for use in labeling primers are discussed further in the application in Section III.

The invention further includes a pair of primers for amplifying a SARS polynucleotide sequence comprising (i) a first primer comprising a sequence which is substantially identical to a portion of the complement of a portion of a sequence selected from the group consisting of SEQ ID NO: 7292, SEQ ID NO: 7293, SEQ ID NO: 7294, SEQ ID NO: 7295, SEQ ID NO: 7296, SEQ ID NO: 7297, SEQ ID NO: 7298, SEQ ID NO: 7299, SEQ ID NO: 7300 and SEQ ID NO: 7301 and (ii) a second primer comprising a sequence which is substantially complementary to a portion of the complement of a sequence selected from the group consisting of the sequence SEQ ID NO: 1 and the sequence SEQ ID NO: 2, such that the primer pair defines a template sequence within a sequence selected from the group consisting of the sequence SEQ ID NO: 1 and the sequence SEQ ID NO: 2. The amplicon defined by said first and second primers is preferably between 50 and 250 nucleotides in length. The primers may optionally be labeled to facilitate their detection. Methods and compositions for use in labeling primers are discussed further in the application in Section III.

The invention includes a kit comprising (i) a first primer comprising a sequence which is substantially identical to a portion of a sequence selected from the group consisting of SEQ ID NO: 7292, SEQ ID NO: 7293, SEQ ID NO: 7294, SEQ ID NO: 7295, SEQ ID NO: 7296, SEQ ID NO: 7297, SEQ ID NO: 7298, SEQ ID NO: 7299, SEQ ID NO: 7300 and SEQ ID NO: 7301 and (ii) a second primer comprising a sequence which is substantially complementary to a portion of a sequence selected from the group consisting of the sequence SEQ ID NO: 1 and the sequence SEQ ID NO: 2, such that the primer pair (i) and (ii) defines a template sequence within a sequence from the group consisting of SEQ ID NO: 1 and SEQ ID NO: 2. Preferably, the (i) first primer comprises a sequence which is substantially identical to a portion of a sequence selected from the group consisting of SEQ ID NO: 7292 and SEQ ID NO: 7293. Preferably, the (i) first primer comprises a sequence which is substantially identical to a portion of the sequence of SEQ ID NO: 7293. The primers may optionally be labeled to facilitate their detection. Methods and compositions for use in labeling primers are discussed further in the application in Section III.

Other preferred kits comprise (i) a first primer comprising a sequence which is substantially identical to a portion of the complement of a portion of a sequence selected from the group consisting of SEQ ID NO: 7292, SEQ ID NO: 7293, SEQ ID NO: 7294, SEQ ID NO: 7295, SEQ ID NO: 7296, SEQ ID NO: 7297, SEQ ID NO: 7298, SEQ ID NO: 7299, SEQ ID NO: 7300 and SEQ ID NO: 7301 and (ii) a second primer comprising a sequence which is substantially complementary to a portion of the complement of a sequence selected from the group consisting of SEQ ID NO: 1 and SEQ ID NO: 2, such that the primer pair defines a template sequence within a sequence selected from the group consisting of SEQ ID NO: 1 and SEQ ID NO: 2.

The invention further includes an attenuated SARS virus for use as a vaccine wherein the intergenic region has been mutated to reduce expression of the viral structural or nonstructural proteins. The attenuated SARS virus may comprises one or more additions, deletions or insertion in one or more of the intergenic regions of the viral genome. Preferably, the attenuated SARS virus comprises an addition, deletion or insertion in one or more occurrences of the sequence selected from the group consisting of SEQ ID NO: 7292 and SEQ ID NO: 7293. Preferably, the addition, deletion or insertion occurs in one or more occurrences of SEQ ID NO: 7293.

The invention further comprises a small molecule which inhibits binding or association of the SARS viral replication machinery, such as a ribonucleoprotein, with the intergenic region of the viral genome. Preferably, the small molecule inhibits binding or association of the SARS viral machinery with a sequence selected from the group consisting of SEQ ID NO: 7292 and SEQ ID NO: 7293. Preferably, the small molecule inhbiits binding or association of the SARS viral machinery with SEQ ID NO: 7293. The invention further includes a method of screening for a small molecule for treatment of SARS viral infection comprising using an assay to identify a small molecule which interferes with the association of the SARS viral replication machinery with the intergenic region of the SARS viral genome.

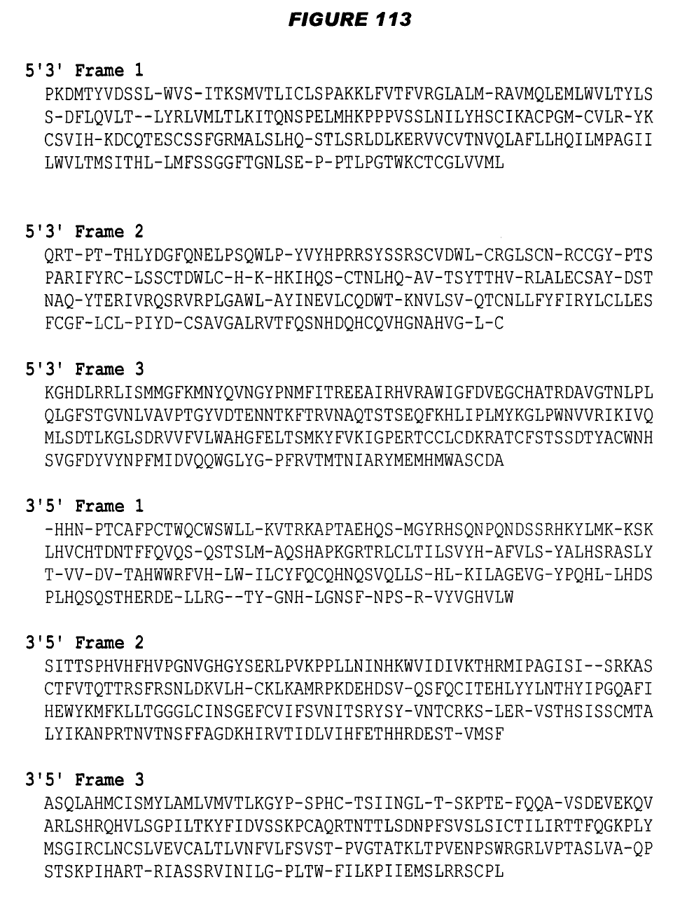

The invention further provides a novel SARS polynucleotide sequence SEQ ID NO: 9968. All six reading frames of this 690 mer sequence are shown in FIG. 113. The constituent amino acid sequences from FIG. 113, having at least 4 amino acids, are listed as SEQ ID NOS: 9969 to 10032.

Accordingly the invention includes a polynucleotide sequence comprising SEQ ID NO: 9968. It also provides polynucleotide sequences having sequence identity to SEQ ID NO: 9968. The degree of sequence identity is preferably greater than 50% (e.g., 60%, 70%, 80%, 85%, 88%, 90%, 92%, 95%, 99% or more).

The invention includes an amino acid sequence encoded by the polynucleotide sequence of SEQ ID NO: 9968, including the amino acid sequences selected from the group consisting of SEQ ID NO^{S}: 9969 to 10032. Preferably, the amino acid sequence comprises SEQ ID NO: 9997 or comprises SEQ ID NO: 9998.

The invention also provides amino acid sequences having sequence identity to an amino acid sequence encoded by SEQ ID NO: 9968. The invention provides amino acids having sequence identity to an amino acid sequence selected from the group consisting of SEQ ID NO^{S}: 9969 to 10032. The degree of sequence identity is preferably greater than 50% (e.g., 60%, 70%, 80%, 85%, 88%, 90%, 92%, 95%, 99% or more).

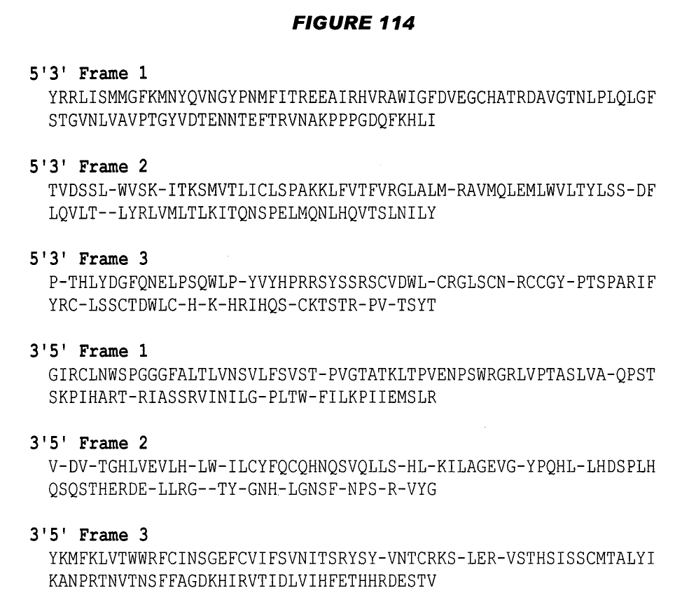

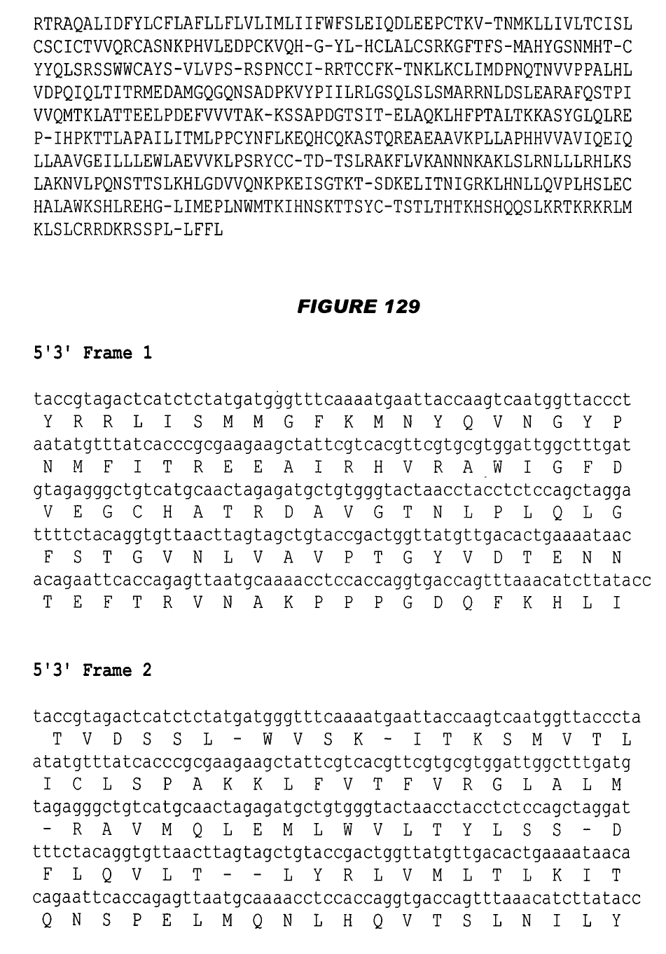

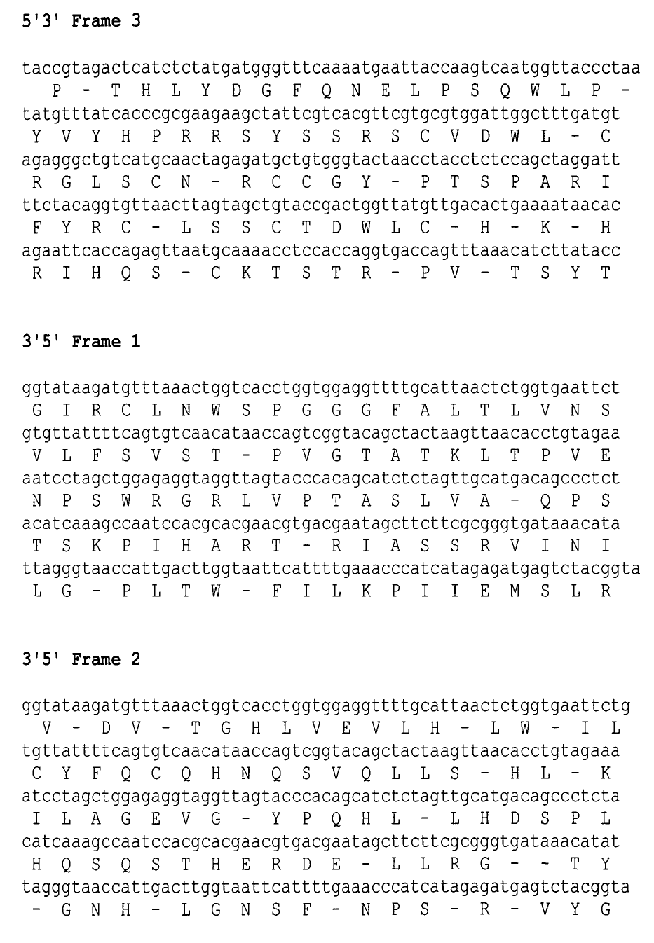

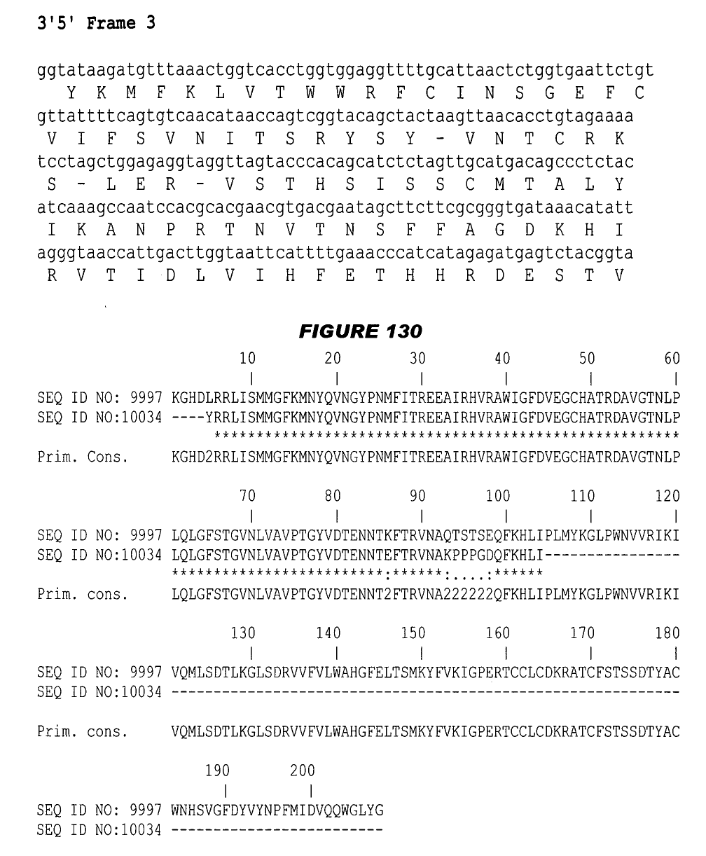

A portion of SEQ ID NO: 9968 matches with approximately 98% identity to a previously published SARS polynucleotide sequence, commonly referred to as “BNI-1” (SEQ ID NO: 10033). BNI-1 was sequenced at Bernhard Nocht Institute for Tropical Medicine, National Reference Center for Tropical Infectious Diseases in Hamburg, Germany. The BNI-1 sequence was published on the WHO website on Apr. 4, 2003 at http://www.who.int/csr/sars/primers/en and in Dorsten et al., “Identification of a Novel Coronavirus in Patients with Severe Acute Respiratory Syndrome”, New England Journal of Medicine, published online at http://www.nejm.org on Apr. 10, 2003. Both references are incorporated herein by reference in their entirety. The six reading frames of this 302 mer sequence are shown in FIG. 114 (see also FIG. 129). The constituent amino acid sequences from FIG. 114, having at least 4 amino acids, are listed as SEQ ID NO_{S}: 10034 to 10065. An alignment of SEQ ID NO: 10034 with SEQ ID NO: 9997 is shown in FIG. 130.

The invention provides for polynucleotide sequences comprising fragments of SEQ ID NO: 9968. In one embodiment, the fragment does not consist entirely of SEQ ID NO: 10033 or of a known coronavirus.

The invention provides for amino acid sequences comprising fragments of an amino acid sequence encoded by SEQ ID NO: 9968. In one embodiment, the fragment does not consist entirely of an amino acid sequence encoded by SEQ ID NO: 10033 or a known coronavirus.

The invention provides for amino acids comprising fragments of an amino acid sequence selected from the group consisting of SEQ ID NO^{S}: 9969 to 10032. In one embodiment, the fragment does not consist entirely of an amino acid sequence encoded by the polynucleotide sequence of SEQ ID NO: 10033 or a known coronavirus.

Approximately 100 nucleotides at the 5′ end of SEQ ID NO: 9968 do not match any portion of the BNI-1 polynucleotide sequence (SEQ ID NO: 10033). This unmatched portion is set forth as SEQ ID NO: 10066. The invention thus further provides a polynucleotide comprising the sequence comprising SEQ ID NO: 10066, polynucleotide sequences having sequence identity to SEQ ID NO: 10066, or polynucleotide sequences comprising fragments of SEQ ID NO: 10066.

The invention further comprises an amino acid sequence encoded by SEQ ID NO: 10066, an amino acid sequence having sequence identity to an amino acid sequence encoded by SEQ ID NO: 10066, or an amino acid sequence comprising fragments of an amino acid sequence encoded by SEQ ID NO: 10066. Preferably, the amino acid sequence comprises SEQ ID NO: 10067.

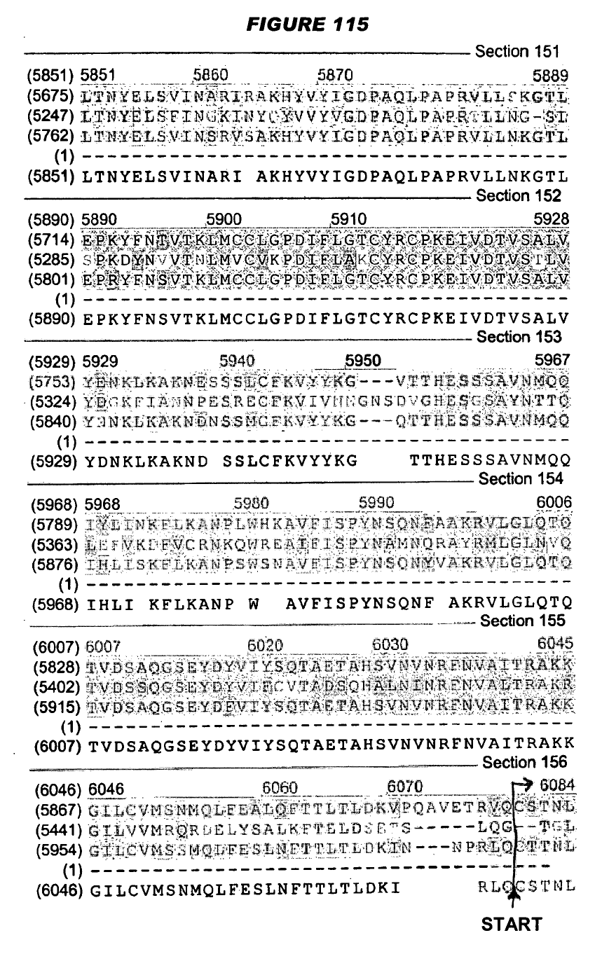

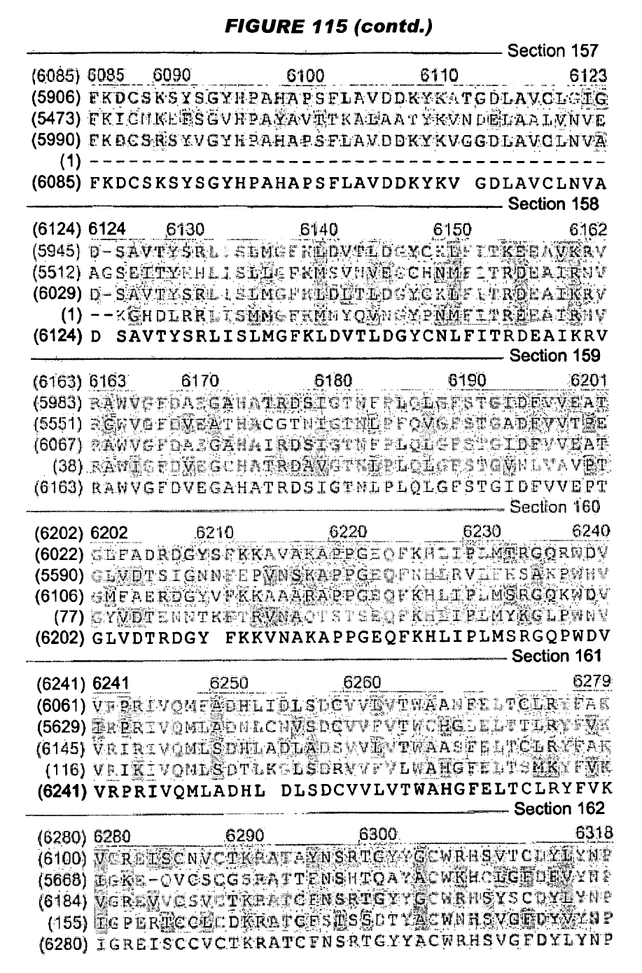

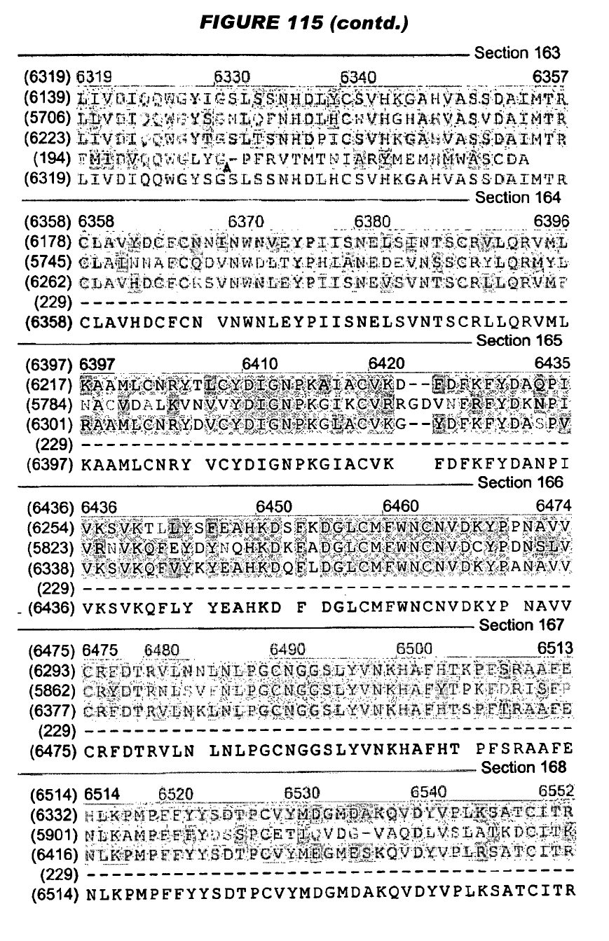

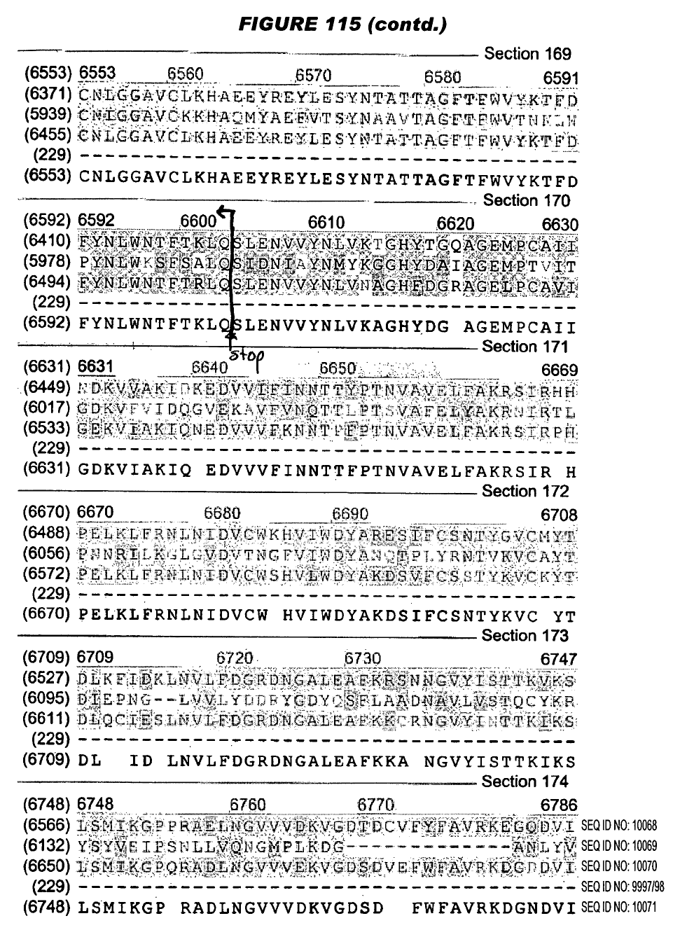

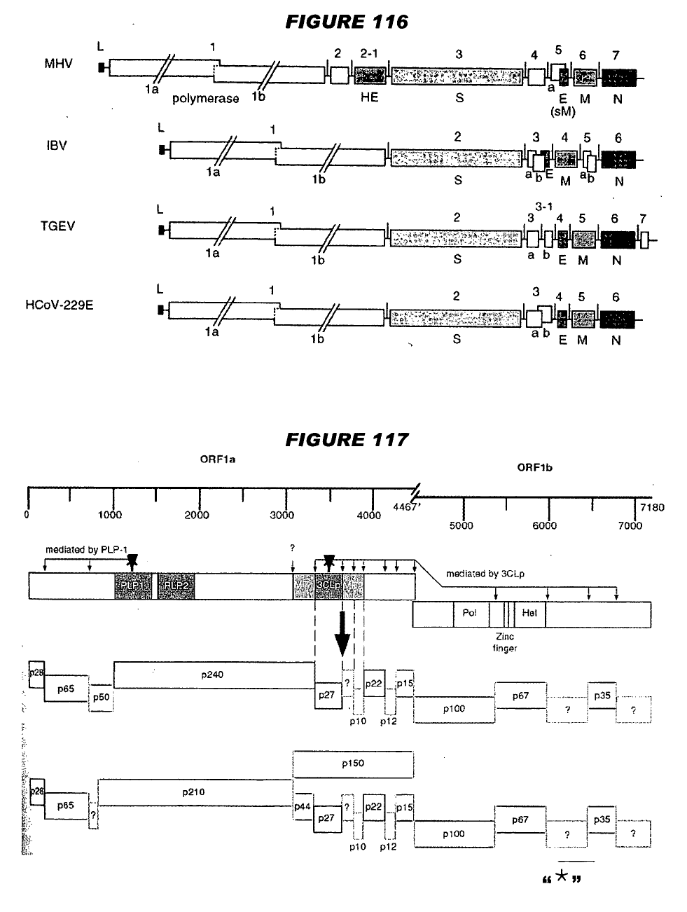

SEQ ID NO: 9997/9998 demonstrates homology with the a region of pol 1ab of several coronaviruses. FIG. 115 shows an alignment of SEQ ID NO^{S}: 9997/9998 to amino acid sequences for pol 1ab of bovine coronavirus (SEQ ID NO: 10068), avian infectious bronchitis virus (SEQ ID NO: 10069) and murine hepatitis virus (SEQ ID NO: 10070). A consensus amino acid sequence of SEQ ID NO^{S}: 9997/9998, SEQ ID NO: 10068, SEQ ID NO: 10069, and SEQ ID NO: 10070 is shown in the bottom row of the alignment in FIG. 115 (e.g. SEQ ID NO: 10071).

As shown in FIG. 113, the polynucleotide sequence encoding SEQ ID NO: 9997 has a stop codon after codon 205, between SEQ ID NO^{S}: 9997 and 9998. Optionally, the stop codon can be removed and the amino acid sequence continued (SEQ ID NO: 10072). Accordingly, the invention provides for an amino acid sequence comprising SEQ ID NO: 9997 and/or SEQ ID NO: 9998, or SEQ ID NO: 10072, and further comprising an amino acid sequence encoding for the C-terminus of a coronavirus pol 1ab gene or a fragment thereof.

As shown in FIG. 115, SEQ ID NO^{S}: 10068, 10069, 10070 and 10071 contain amino acids prior to the N-terminus of SEQ ID NO: 9997. The invention also provides for an amino acid sequence comprising SEQ ID NO: 9997 and further comprising an amino acid sequence encoding for the N-terminus of a coronavirus pol1ab protein or a fragment thereof.

The pol1ab sequences on FIG. 115 contain a coding region indicated on the schematic of FIG. 117 by a “*”. In FIG. 115, the beginning of this genomic region is designated by the arrow crossing in front of amino acid 6080 of the consensus sequence SEQ ID NO: 10071. The end of this genomic region is designated by the arrow crossing in front of amino acid 6604 of the consensus sequence. The invention provides for an amino acid sequence comprising SEQ ID NO: 9997 and/or SEQ ID NO: 9998, or SEQ ID NO: 10072, and further comprising a first amino sequence prior to the N-terminus of said SEQ ID NO: 9997 and/or SEQ ID NO: 9998, or SEQ ID NO: 10072, wherein said first amino acid sequence has homology to an N-terminus sequence of a known coronavirus pol 1ab “*” protein or a fragment thereof.

The invention further provides for an amino acid sequence comprising SEQ ID NO: 9997 and SEQ ID NO: 9998, wherein the stop codon after SEQ ID NO: 9971 is removed (i.e. SEQ ID NO: 10072), and further comprising a second amino acid sequence following the C terminus of SEQ ID NO: 9998, wherein said second amino acid sequence is homologous with a C terminus of a known coronavirus pol 1ab “*” protein or a fragment thereof.

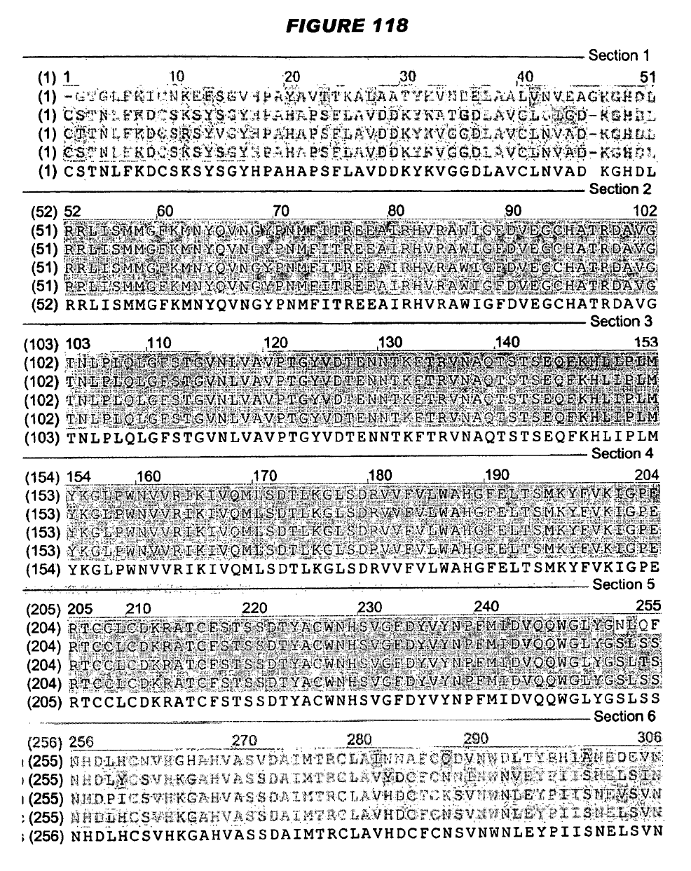

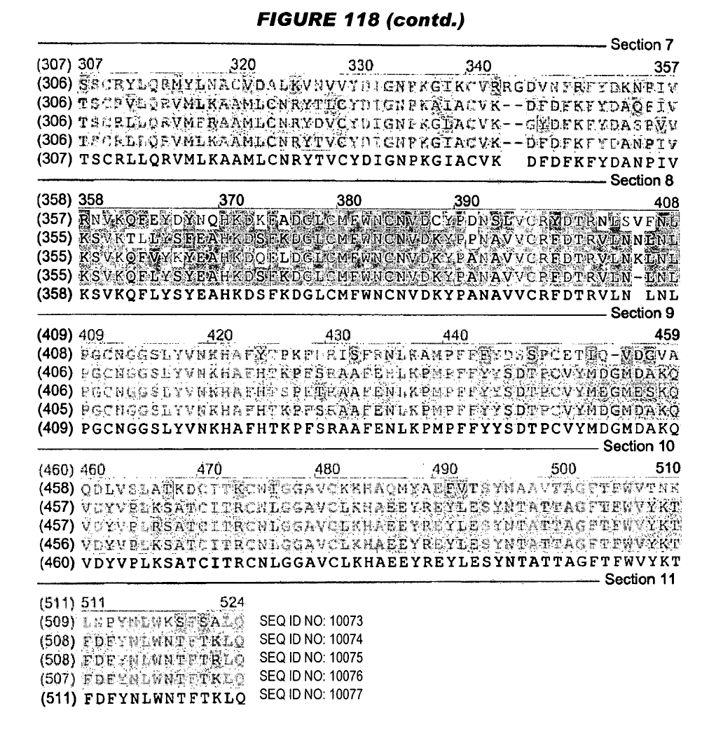

Examples of such proteins are shown aligned in FIG. 118, and are SEQ ID NO^{S}: 10073 to 10077. SEQ ID NO: 10073 comprises SEQ ID NO: 9997 and further comprises amino acids prior to the N-terminus and subsequent to the C-terminus from the pol 1ab “*” protein of avian infectious bronchitis virus. SEQ ID NO: 10074 comprises SEQ ID NO: 9997 and further comprises amino acids prior to the N-terminus and subsequent to the C-terminus from the pol1ab “*” protein of bovine coronavirus. SEQ ID NO: 10075 comprises SEQ ID NO: 9997 and further comprises amino acids prior to the N-terminus and subsequent to the C-terminus from the pol 1ab “*” protein of murine hepatitis virus. SEQ ID NO: 10076 comprises SEQ ID NO: 9997 and further comprises amino acids prior to the N-terminus and subsequent to the C-terminus from the consensus of the pol1ab “*” protein of avian infectious bronchitis virus, bovine coronavirus, and murine hepatitis virus (FIG. 115). SEQ ID NO: 10077 comprises the consensus sequence of SEQ ID NOS: 10073 to 10076.

The invention comprises an amino acid sequence selected from the group consisting of SEQ ID NO^{S}: 10073, 10074, 10075, 10076 and 10077. The invention further includes an amino acid sequence comprising fragments of an amino acid sequence selected from the group consisting of SEQ ID NO^{S}: 10073, 10074, 10075, 10076 and 10077. The invention further comprises an amino acid sequence with sequence identity to a sequence selected from the group consisting of SEQ ID NO^{S}: 10073, 10074, 10075, 10076 and 10077.

The invention comprises polynucleotides encoding for the amino acid sequences selected from the group consisting of SEQ ID NO^{S}: 10073, 10074, 10075, 10076 and 10077. The invention comprises polynucleotides having sequence identity to polynucleotides encoding for the amino acid sequences selected from the group consisting of SEQ ID NO^{S}: 10073, 10074, 10075, 10076 and 10077. The invention comprises fragments of polynucleotides encoding SEQ ID NO^{S}: 10073, 10074, 10075, 10076 and 10077.

As shown in FIG. 113, SEQ ID NO: 9968 includes a sequence that encodes SEQ ID NO: 10020 followed by a stop codon, giving a C-terminus threonine (Thr) residue. The corresponding sequence from an amino acid sequence encoded by BNI-1 is SEQ ID NO: 10078, which continues past the C-terminus of SEQ ID NO: 10020. Accordingly, the invention includes a protein comprising amino acid sequence SEQ ID NO: 10020 or an amino acid sequence having sequence identity to SEQ ID NO: 10020 or an amino acid sequence comprising a fragment of SEQ ID NO: 10020, wherein the C-terminus residue of said protein is a threonine. Preferably, the C-terminus of said protein is -ST. Still more preferably, the C-terminus of said protein is -EST. The invention also includes a protein comprising amino acid sequence SEQ ID NO: 10078 or an amino acid sequence having sequence identity to SEQ ID NO: 10078 or an amino acid sequence comprising a fragment of SEQ ID NO: 10078, wherein the C-terminus residue of said protein is Thr. Preferably, the C-terminus of said protein is -ST. Still more preferably, the C-terminus of said protein is -EST.

SEQ ID NO: 9968 also encodes a 54 mer amino acid sequence SEQ ID NO: 10015. The polynucleotide encoding SEQ ID NO: 10015 encodes two stop codons at its C-terminus (FIG. 113). The corresponding region from the BNI-1 sequence does not contain this 54 mer. Accordingly, the invention includes a protein comprising amino acid sequence SEQ ID NO: 10015, or an amino acid sequence having sequence identity to SEQ ID NO: 10015 or an amino acid sequence comprising a fragment of SEQ ID NO: 10015. The invention further includes a polypeptide comprising SEQ ID NO: 10015 and further comprising a first amino acid sequence prior to the N-terminus of SEQ ID NO: 10015.

SEQ ID NO: 9968 encodes the amino acid sequence SEQ ID NO: 9969. The polynucleotide sequence contains a stop codon at the C-terminus of SEQ ID NO: 9969. Accordingly, the invention includes a protein comprising amino acid sequence SEQ ID NO: 9969, or an amino acid sequence having sequence identity to SEQ ID NO: 9969. The invention further includes a polypeptide comprising SEQ ID NO: 9969 and further comprising a first amino acid sequence prior to the N-terminus of SEQ ID NO: 9969. The invention further includes a polypeptide comprising the sequence SEQ ID NO: 10079.

SEQ ID NO: 9968 encodes amino acid sequence QRT (FIG. 113), followed by a stop codon. Accordingly, the invention includes a protein comprising amino acid sequence QRT. The invention further includes a polypeptide comprising amino acid sequence QRT and further comprising a first amino acid sequence prior to the N-terminus of the sequence QRT.

SEQ ID NO: 9968 encodes amino acid sequence SEQ ID NO: 10022, followed by a stop codon at its C-terminus. Accordingly, the invention includes a protein comprising amino acid sequence SEQ ID NO: 10022, or an amino acid sequence having sequence identity to SEQ ID NO: 10022. The invention further includes a polypeptide comprising SEQ ID NO: 10022 and further comprising a first amino acid sequence prior to the N-terminus of SEQ ID NO: 10022.

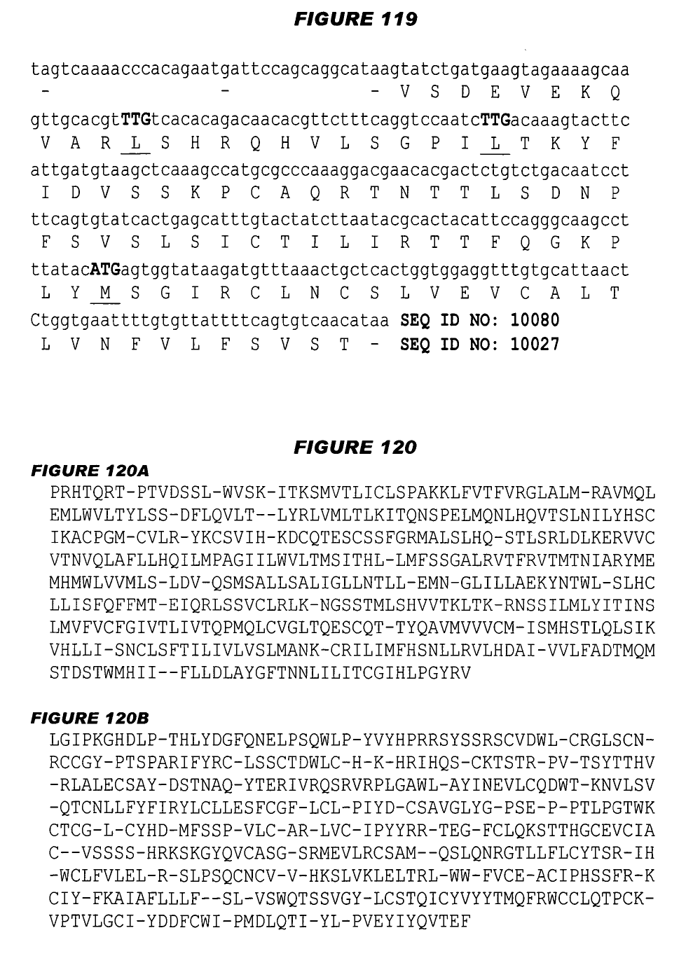

SEQ ID NO: 9968 encodes amino acid sequence SEQ ID NO: 10027. Within the SEQ ID NO: 10027 coding sequence there are at least three start codons, identified with underlining in FIG. 119. The open reading frame indicated by the first start codon is SEQ ID NO: 10081. The open reading frame indicated by the second start codon is SEQ ID NO: 10082. The open reading frame indicated by the third start codon is SEQ ID NO: 10083.

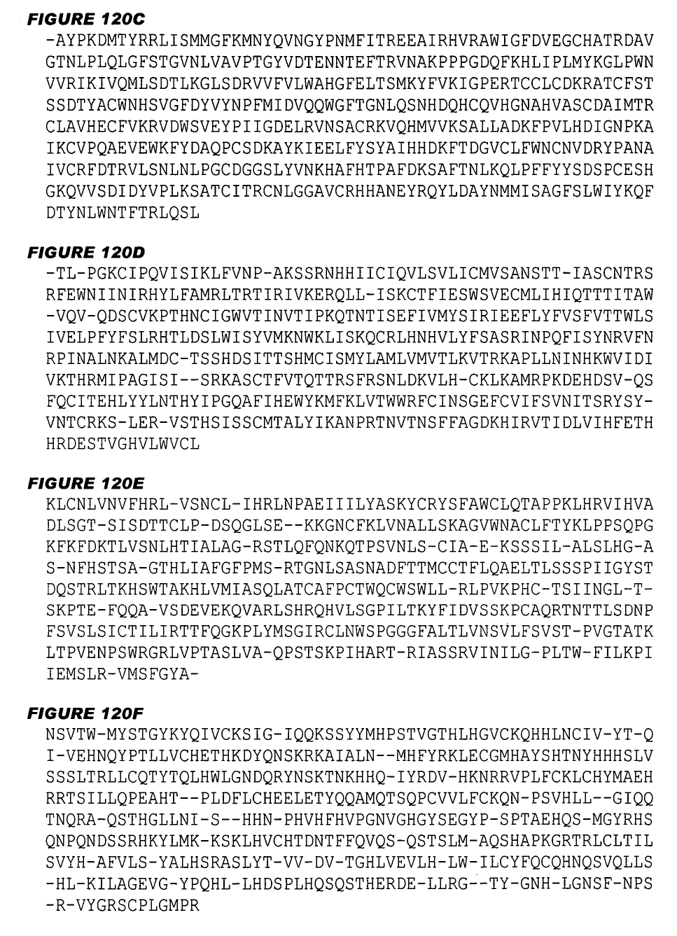

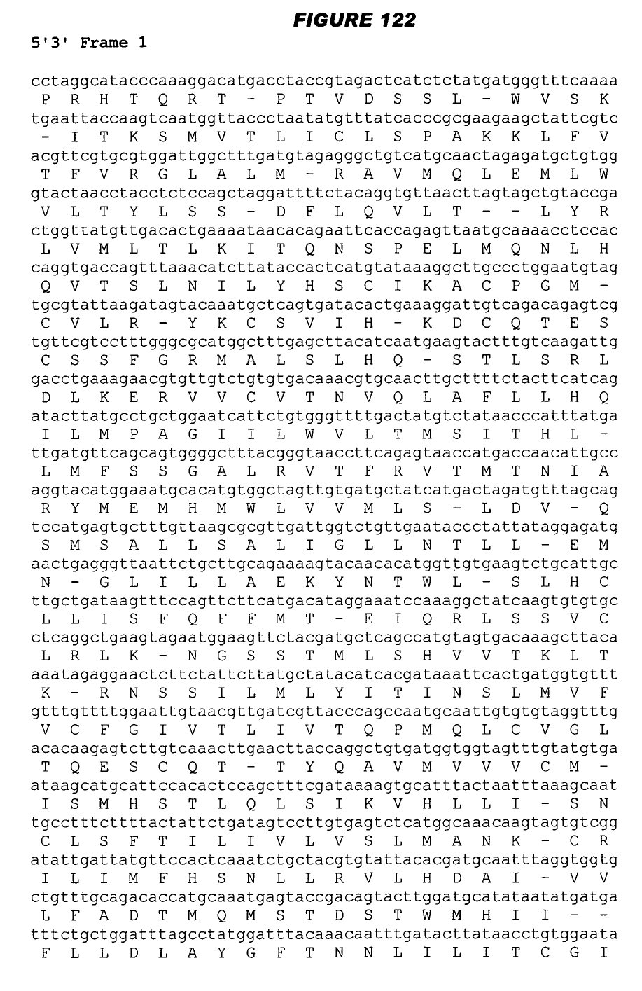

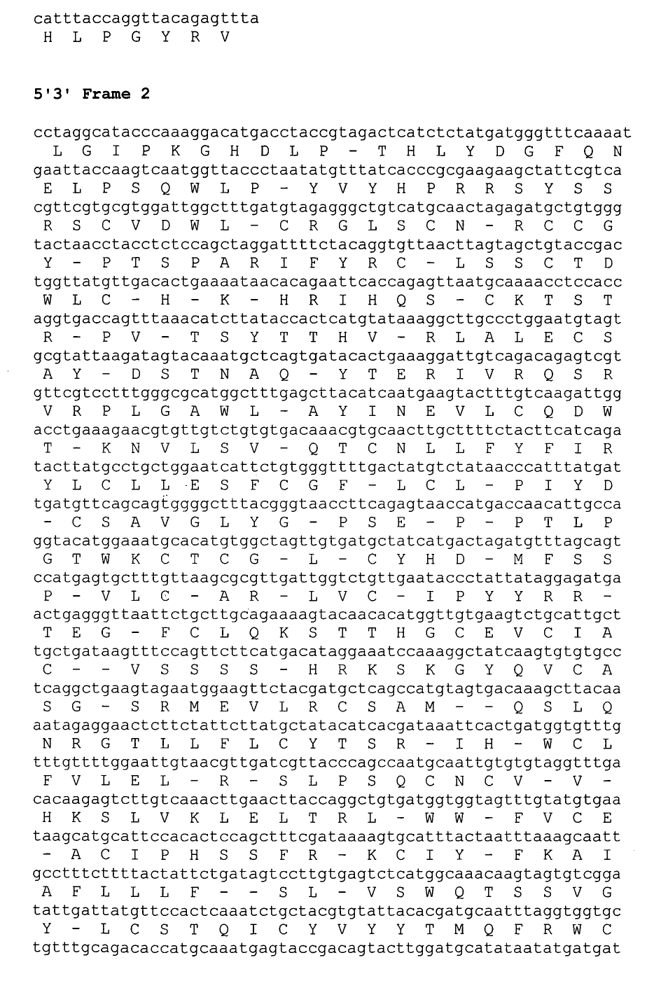

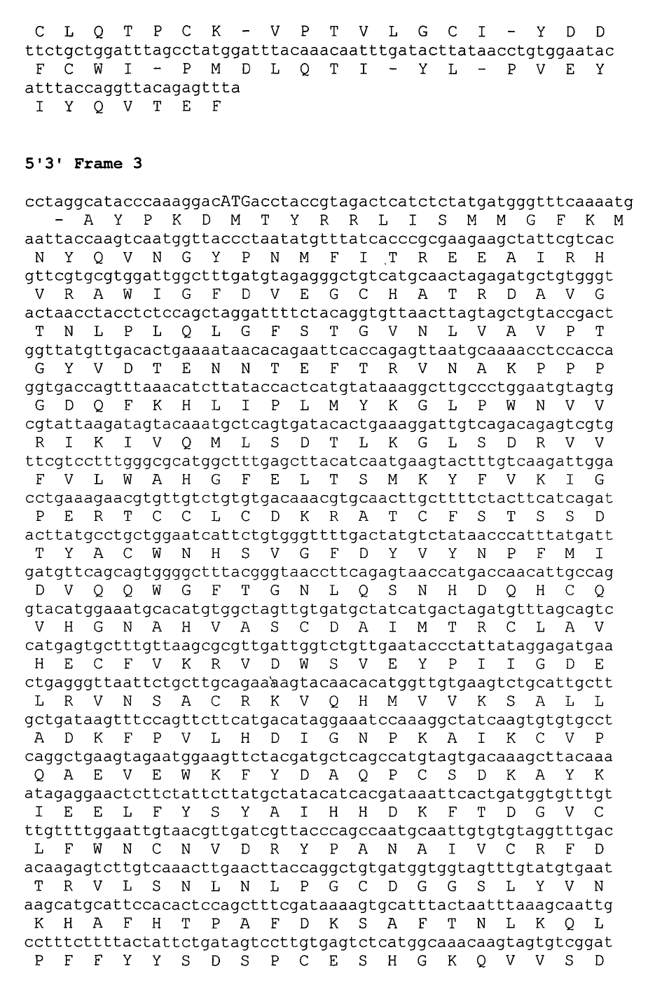

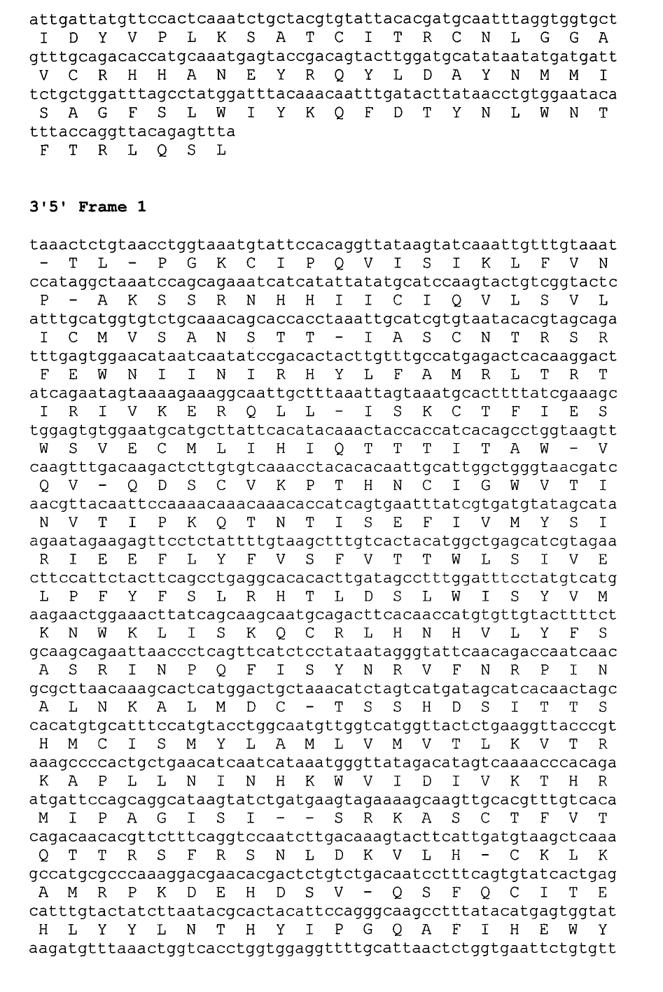

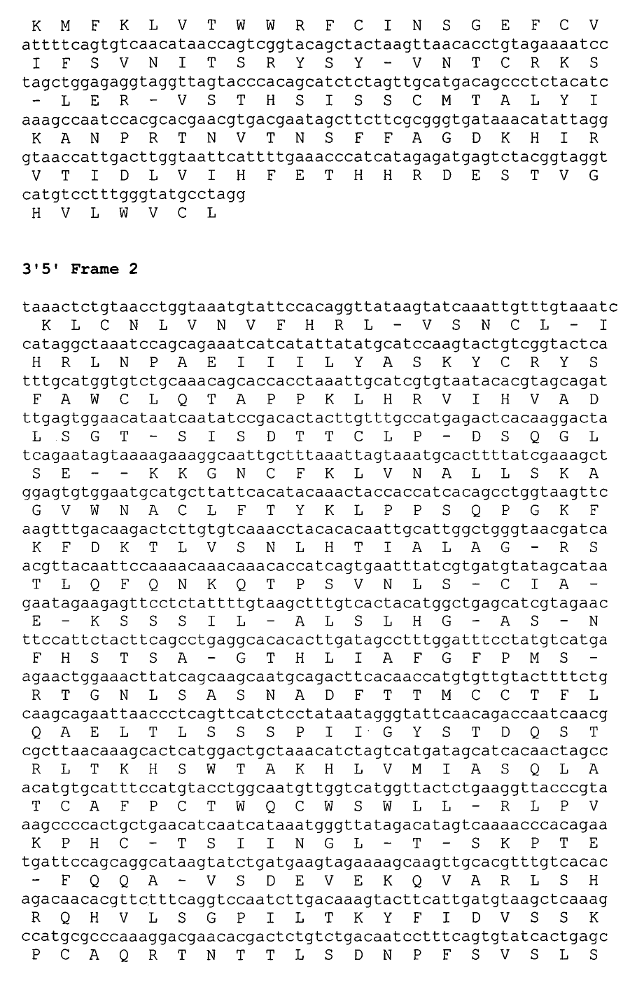

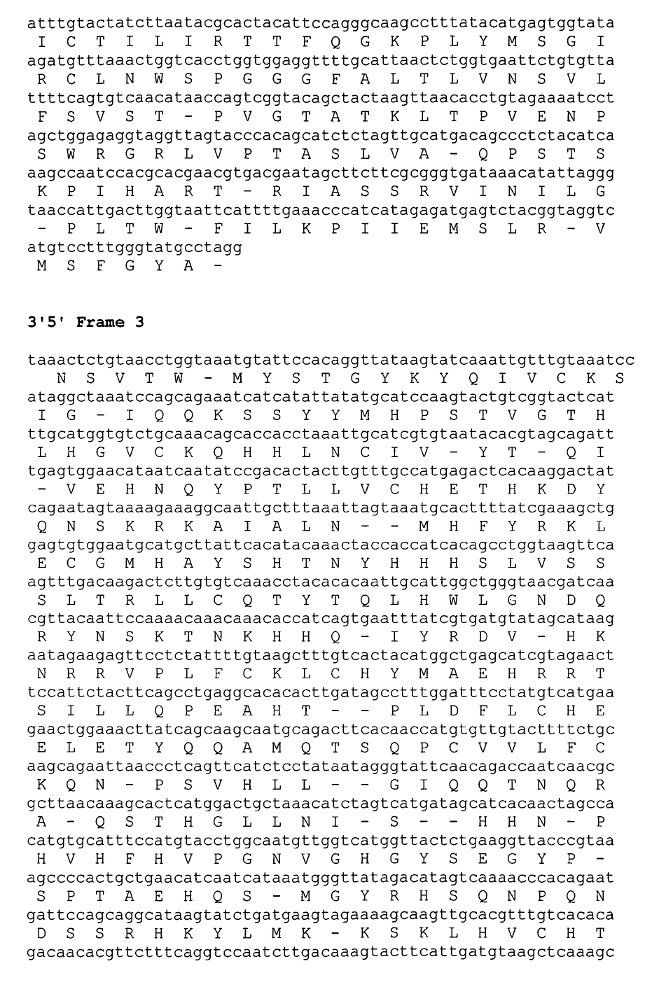

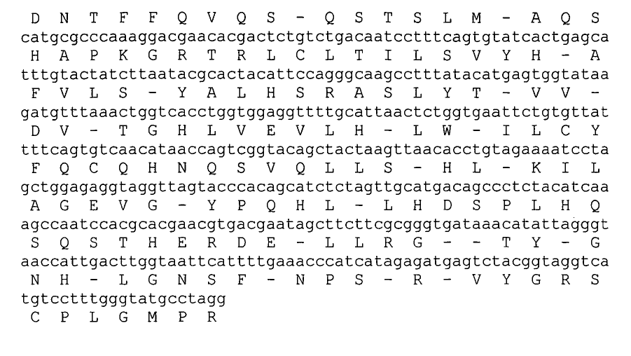

The invention provides a novel SARS polynucleotide sequence SEQ ID NO: 10084. All six reading frames of this 1463 mer sequence are shown in FIG. 120 (see also FIG. 122). The constituent amino acid sequences from FIG. 120, having at least 4 amino acids, are listed as SEQ ID NOS: 10085 to 10209 (see FIGS. 120A to 120F).

The invention includes a polynucleotide sequence comprising SEQ ID NO: 10084. The invention also provides polynucleotide sequences having sequence identity to SEQ ID NO: 10084. The invention also provides for polynucleotide sequences comprising fragments of SEQ ID NO: 10084. In one embodiment, the polynucleotide fragment does not consist entirely of SEQ ID NO: 10033 or a known coronavirus polynucleotide sequence or a known SARS polynucleotide sequence.

The invention includes an amino acid sequence encoded by the polynucleotide sequence of SEQ ID NO: 10084, including the amino acid sequences of FIGS. 120A to 120F e.g. selected from the group consisting of SEQ ID NO^{S}: 10085 to 10209. Preferably, the amino acid sequence comprises SEQ ID NO: 10149.

The invention also provides amino acid sequences having sequence identity to an amino acid sequence encoded by SEQ ID NO: 10084. The invention provides amino acids having sequence identity to an amino acid sequence from FIGS. 120A to 120F e.g. selected from the group consisting of SEQ ID NO^{S}: 10085 to 10209.

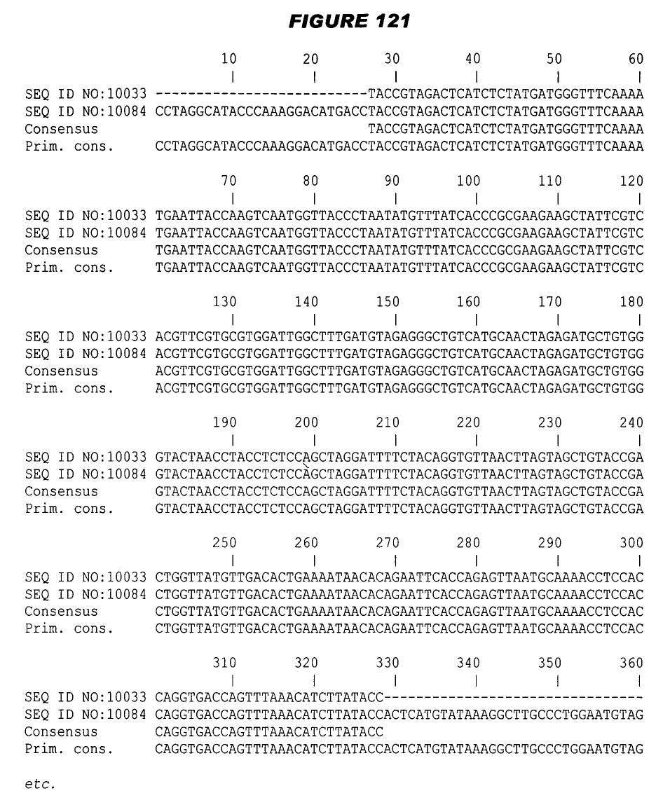

The invention also provides fragments of amino acid sequences encoded by SEQ ID NO: 10084. The invention also provides fragments of amino acid sequences selected from the group consisting of SEQ ID NO^{S}: 10085 to 10209. In one embodiment, the fragment does not consist entirely of an amino acid sequence encoded by SEQ ID NO: 10033 or an amino acid sequence of a known coronavirus or an amino acid sequence of a known SARS virus. An alignment of the matching portion of SEQ ID NO: 10033 and SEQ ID NO: 10084 is included in FIG. 121.

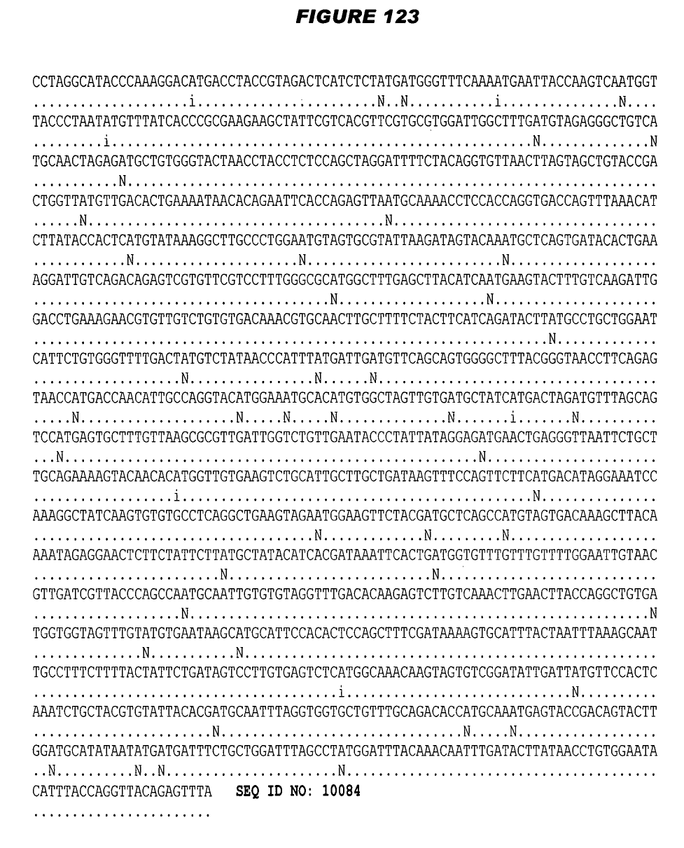

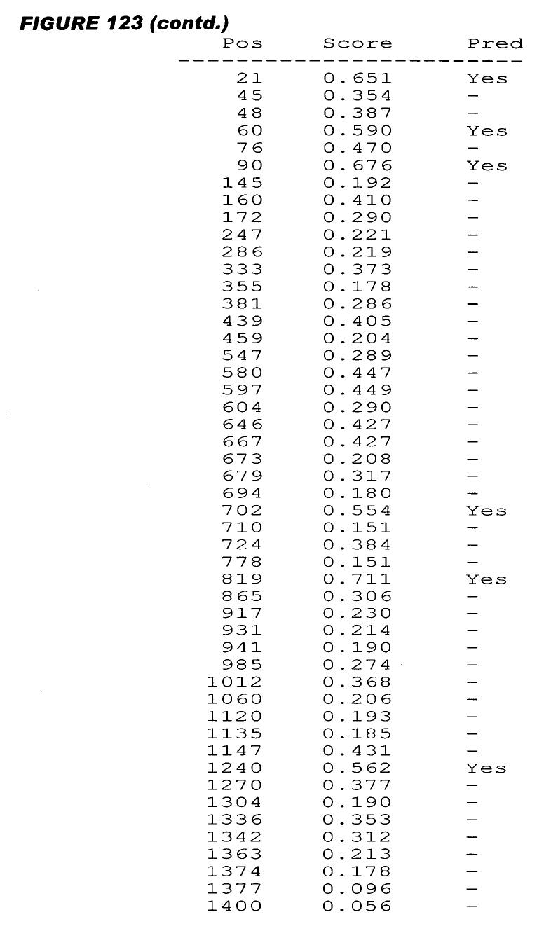

In one embodiment, the invention comprises an amino acid sequence comprising SEQ ID NO: 10149. An alignment of the polynucleotide sequence SEQ ID NO: 10084 to the encoded SEQ ID NO: 10149 is shown in FIG. 122 (5′3′ Frame 3). Analysis of the 5′3′ Frame 3 translation by a computer program to predict start codon methionines (NetStart 1.0) (FIG. 123) reveals SEQ ID NO^{S}: 10210 to 10215.

The invention includes a protein comprising an amino acid sequence selected from the group consisting of SEQ ID NO: 10210, SEQ ID NO: 10211, SEQ ID NO: 10212, SEQ ID NO: 10213, SEQ ID NO: 10214 and SEQ ID NO: 10215. The invention includes a protein having sequence identity to an amino acid sequence selected from the group consisting of SEQ ID NO: 10210, SEQ ID NO: 10211, SEQ ID NO: 10212, SEQ ID NO: 10213, SEQ ID NO: 10214 and SEQ ID NO: 10215. In one embodiment, the protein does not consist entirely of an amino acid sequence of a known SARS virus or of a known coronavirus.

The invention includes a fragment of a protein comprising an amino acid sequence selected from the group consisting of SEQ ID NO: 10210, SEQ ID NO: 10211, SEQ ID NO: 10212, SEQ ID NO: 10213, SEQ ID NO: 10214 and SEQ ID NO: 10215. In one embodiment, the fragment does not consist entirely of an amino acid sequence of a known SARS virus or of a known coronavirus.

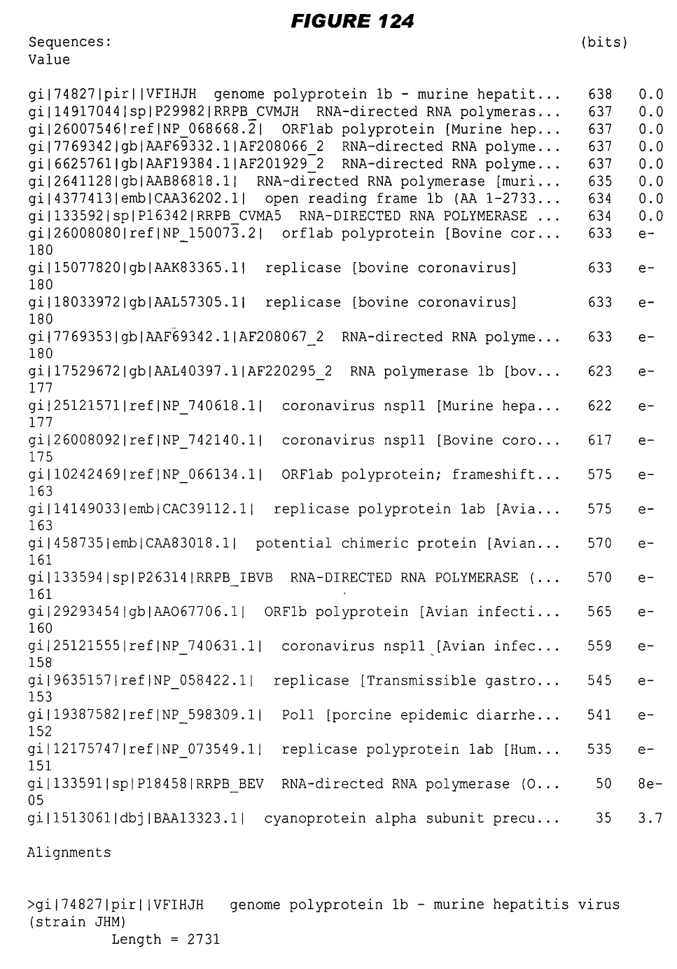

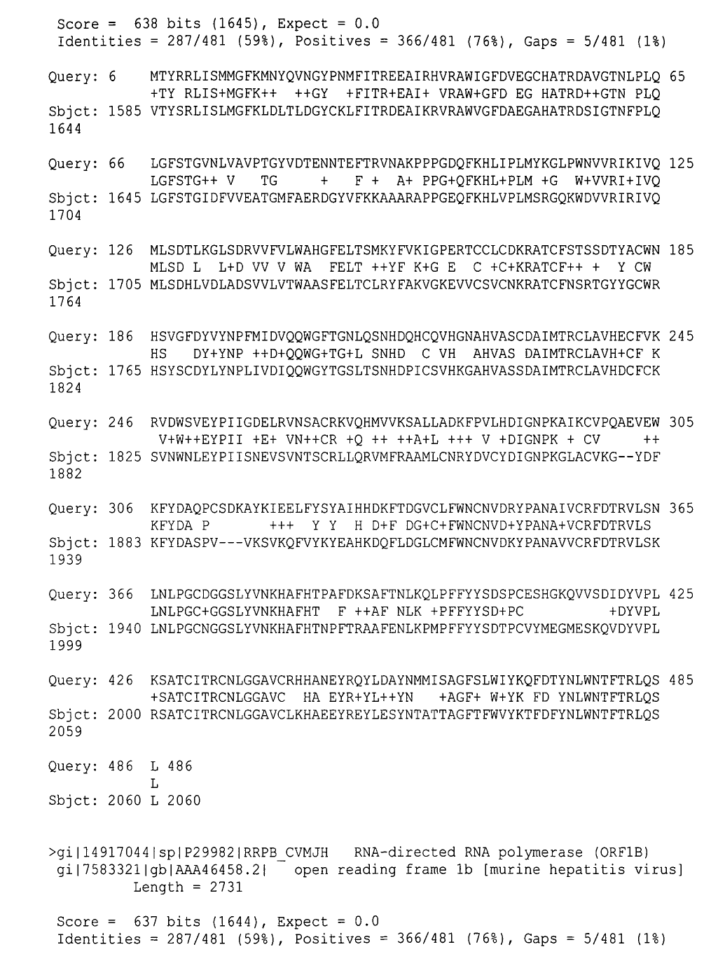

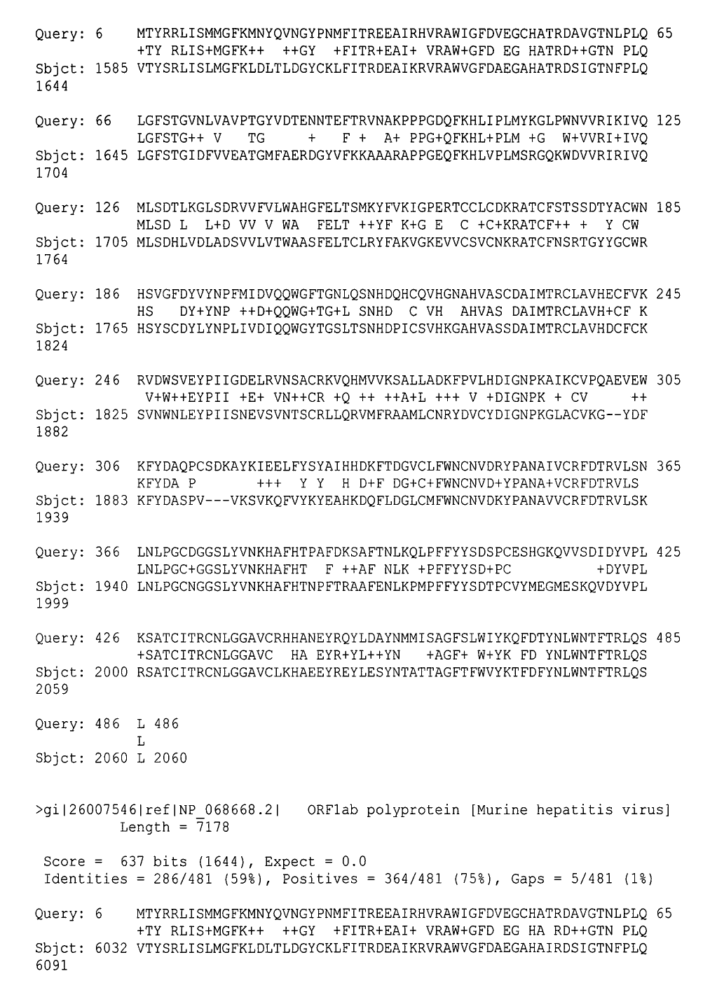

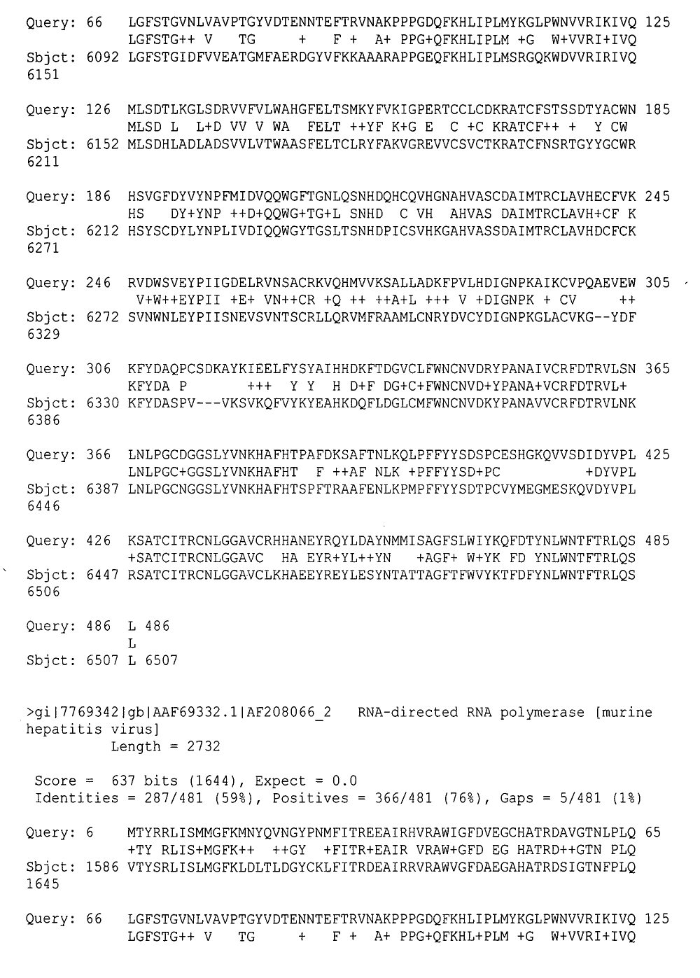

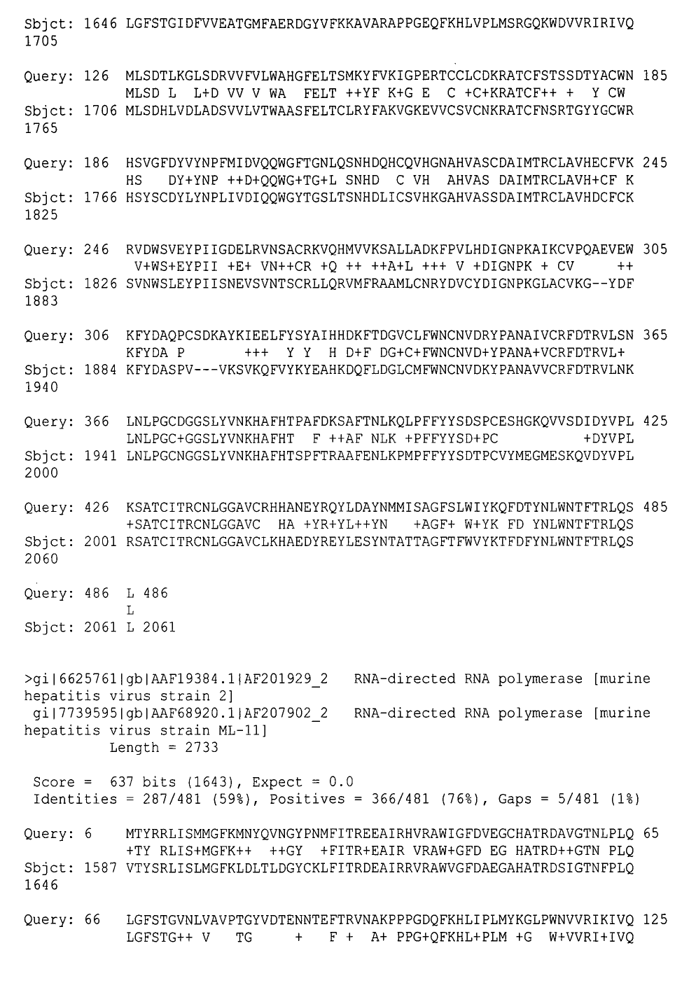

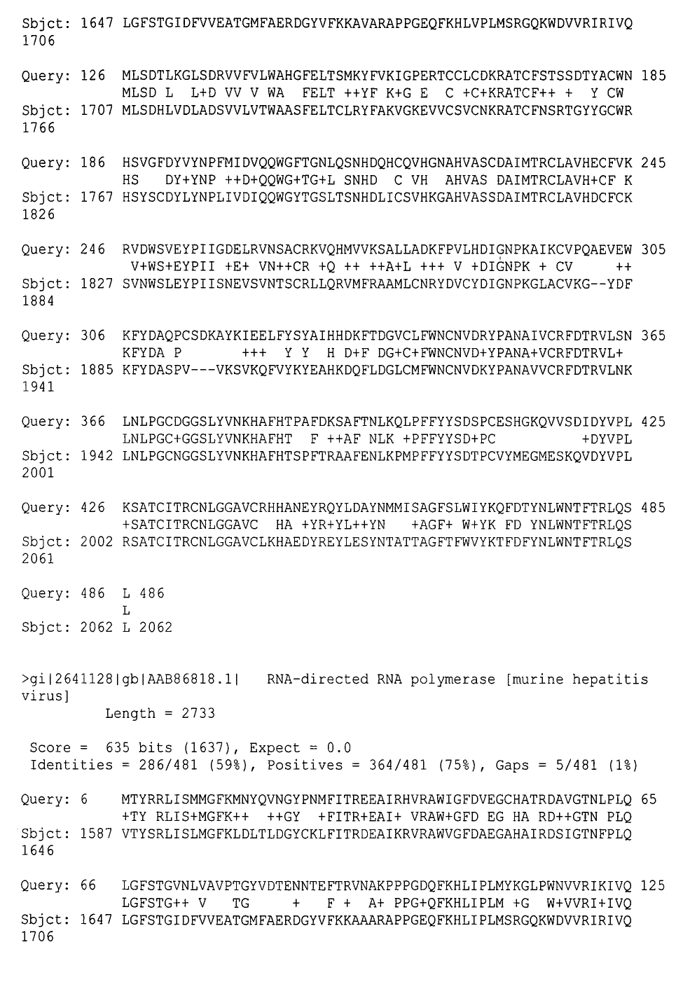

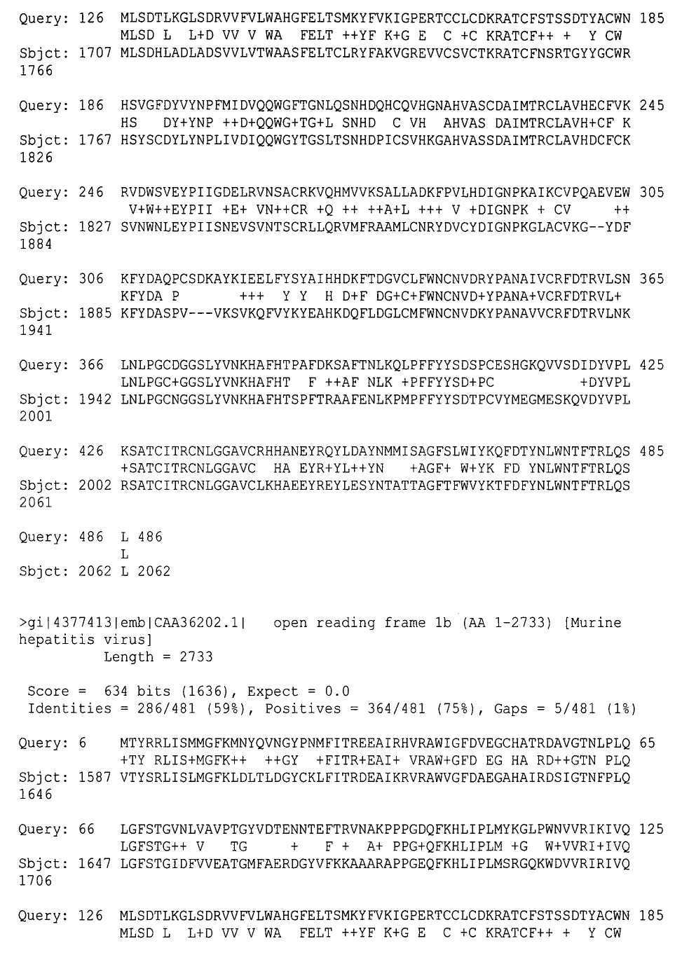

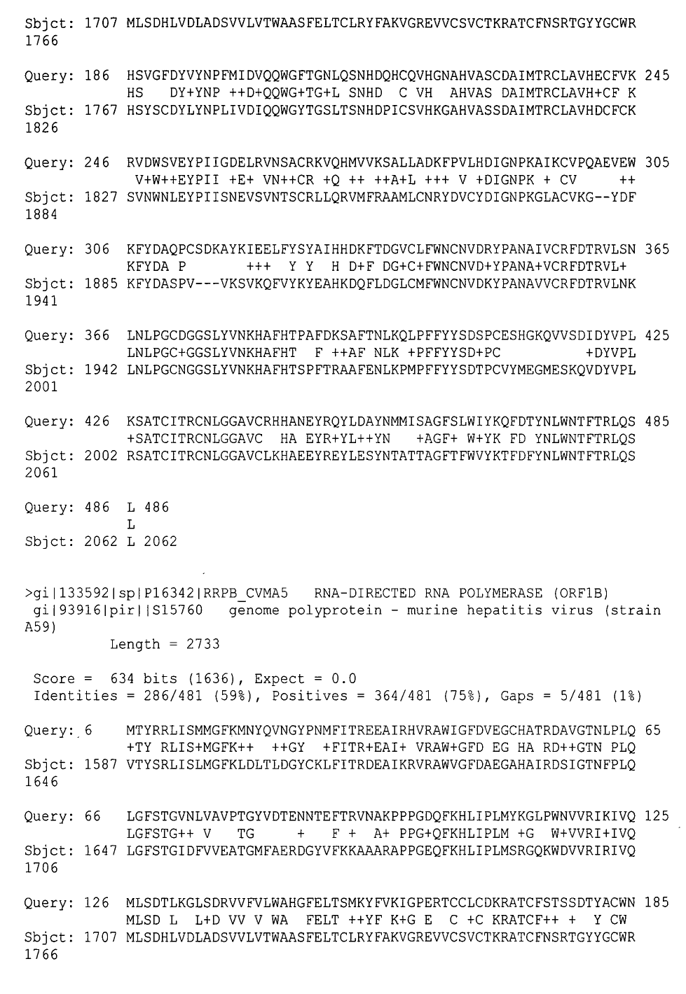

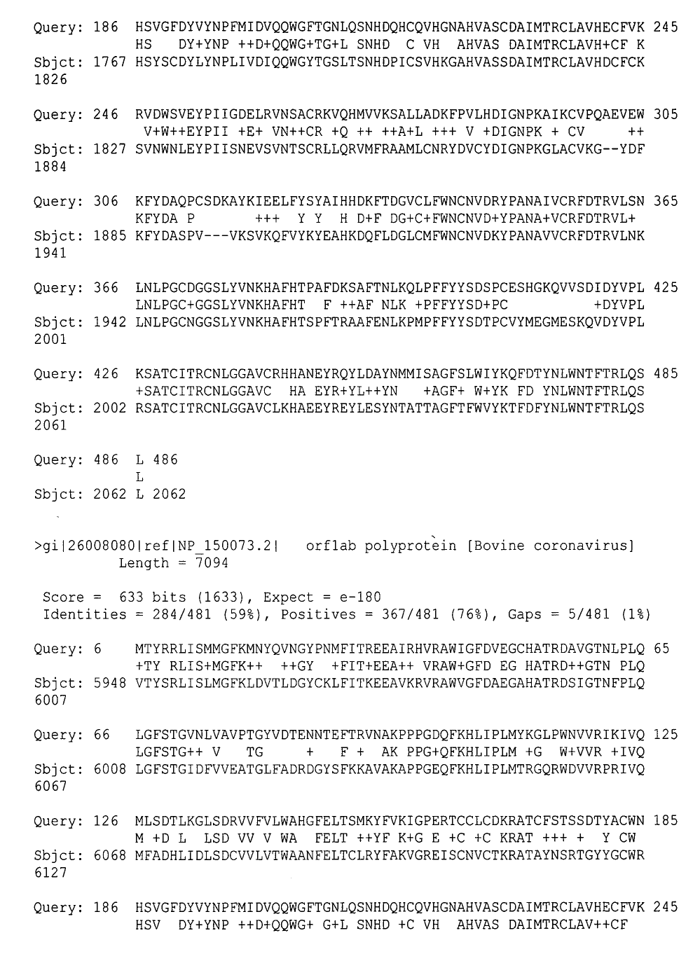

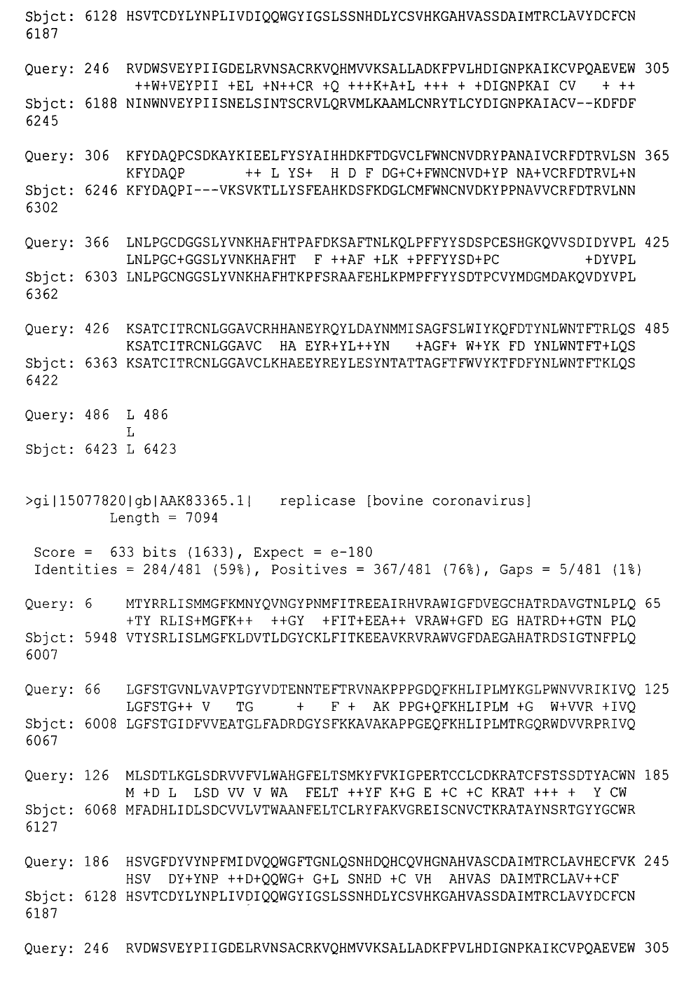

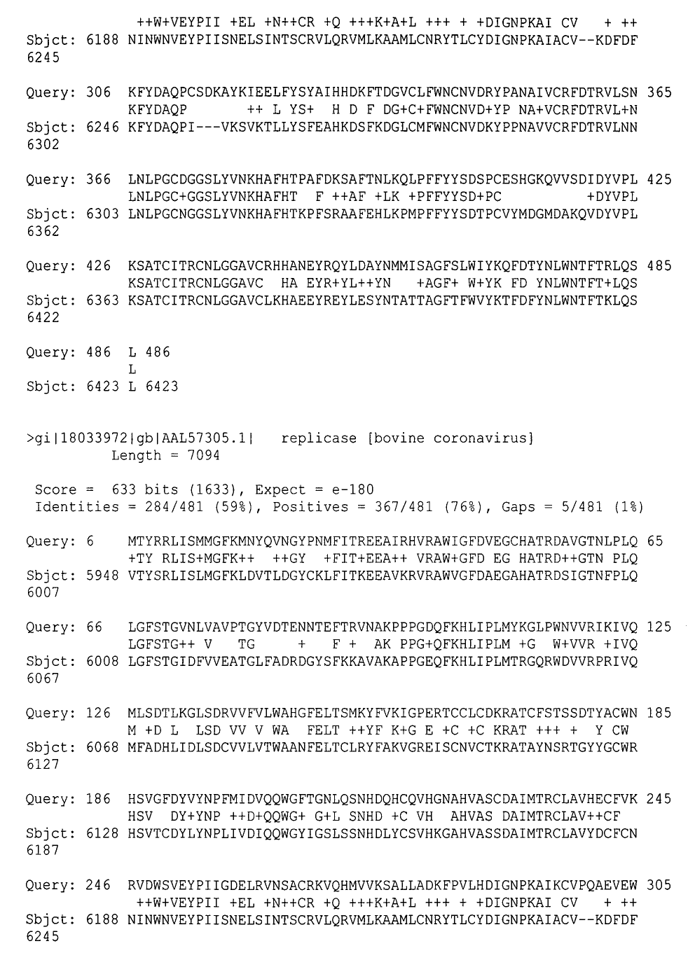

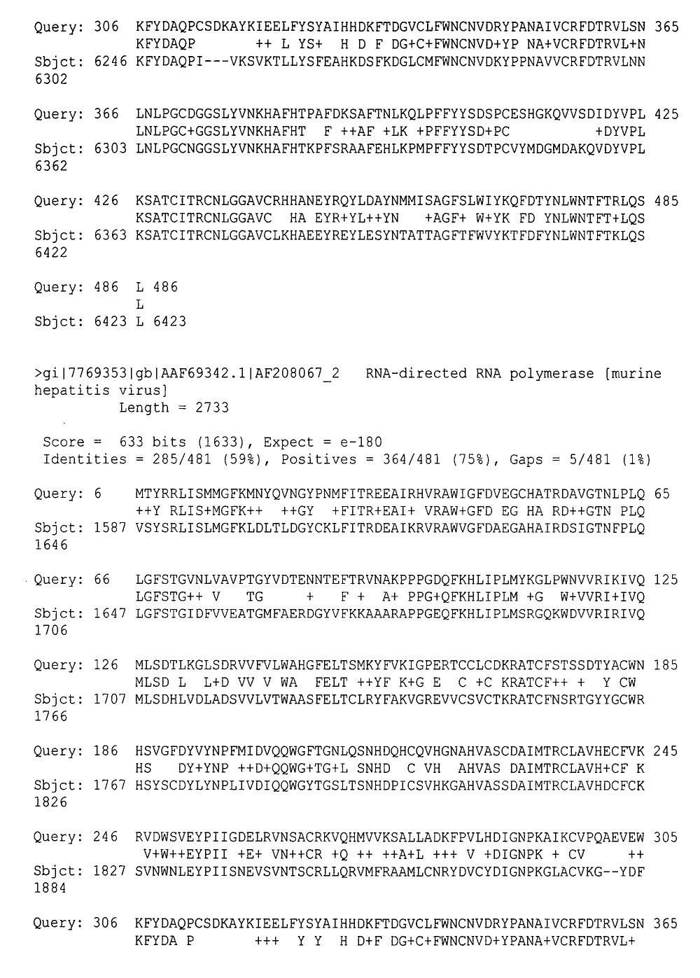

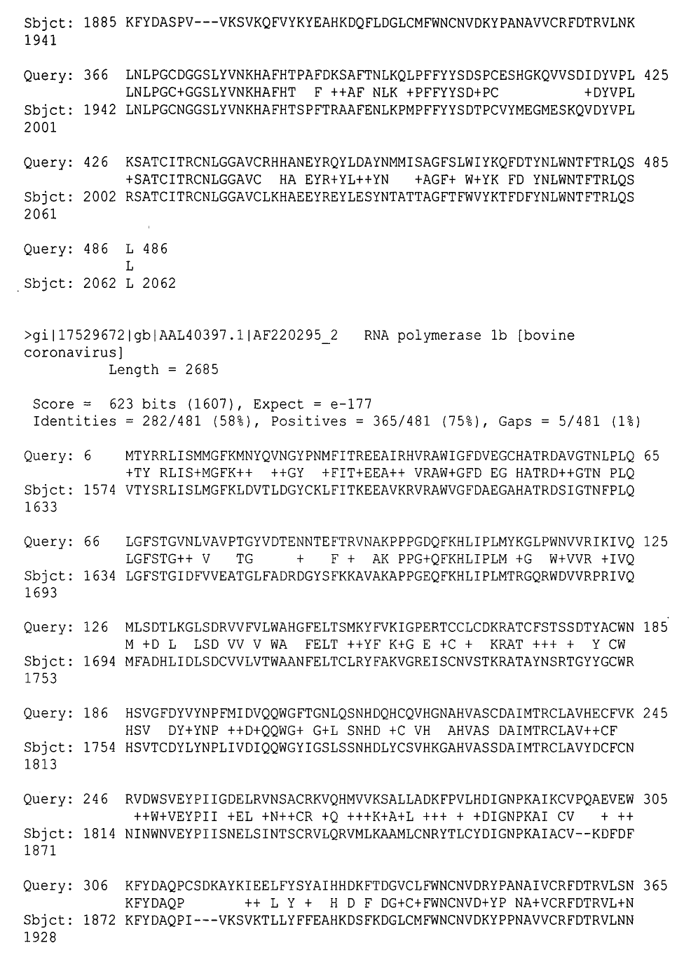

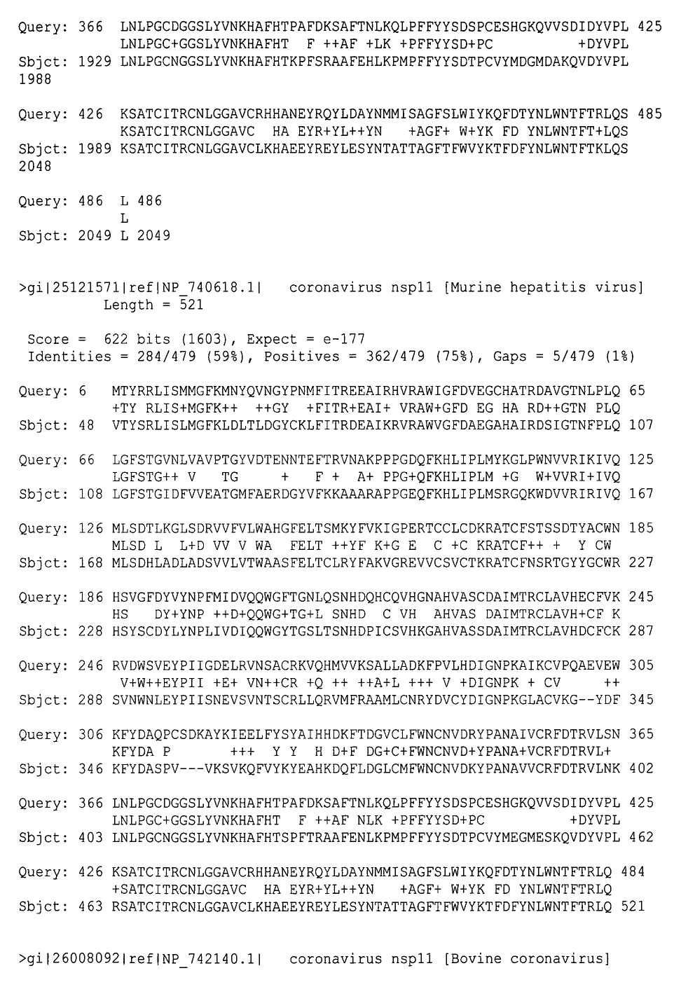

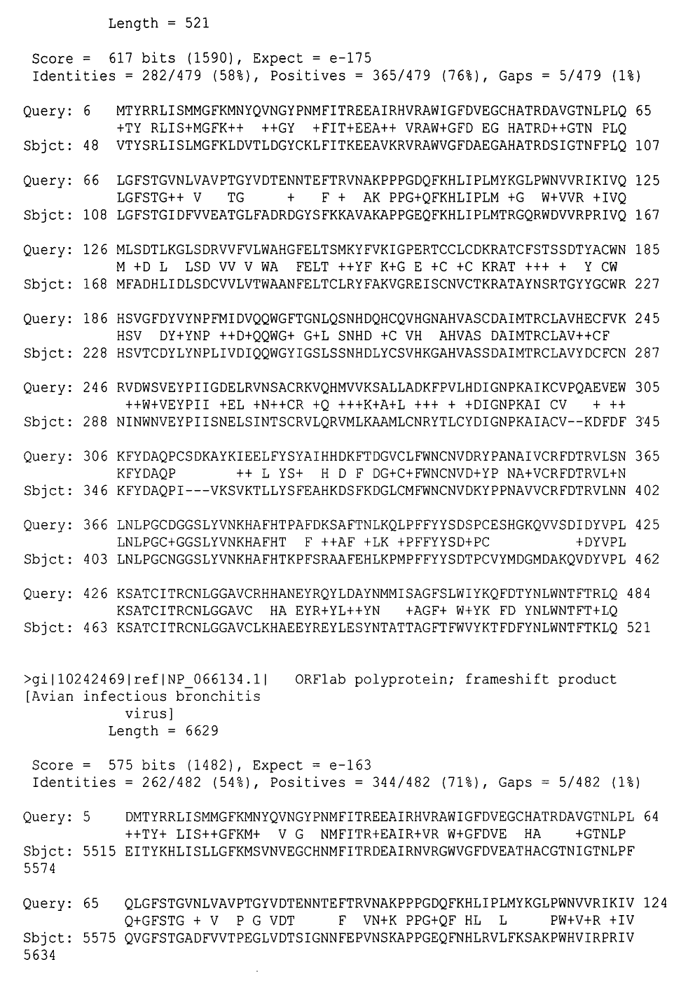

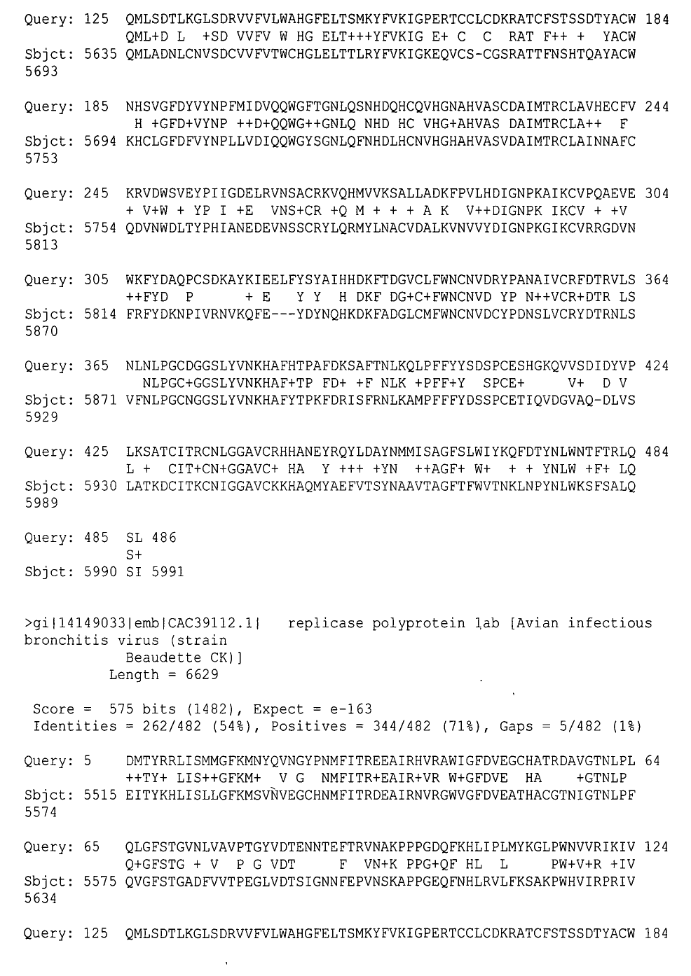

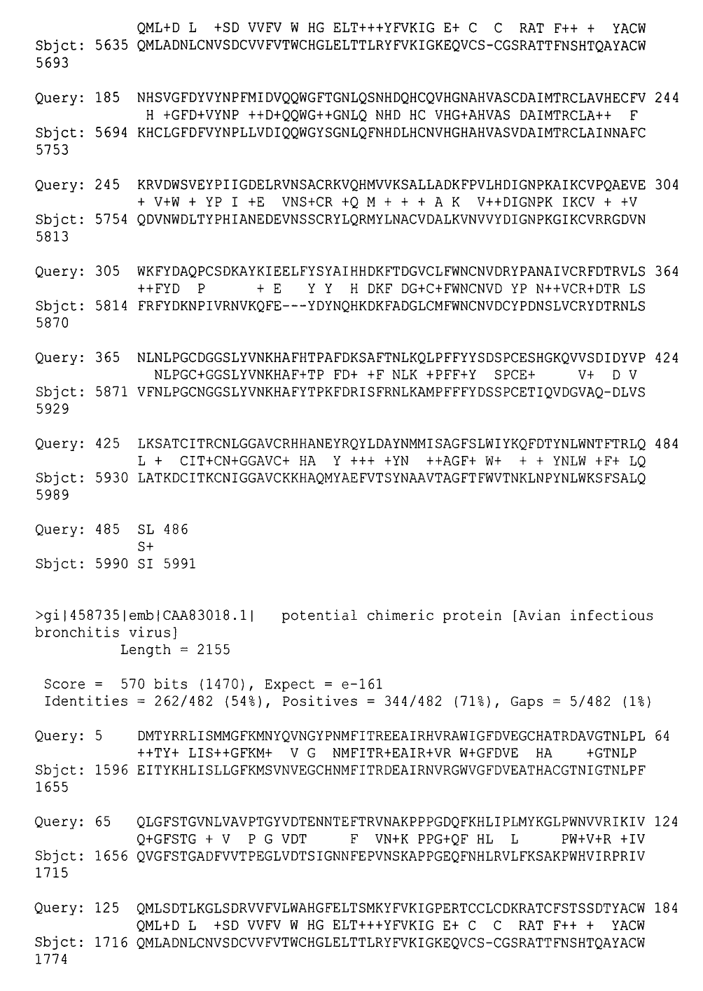

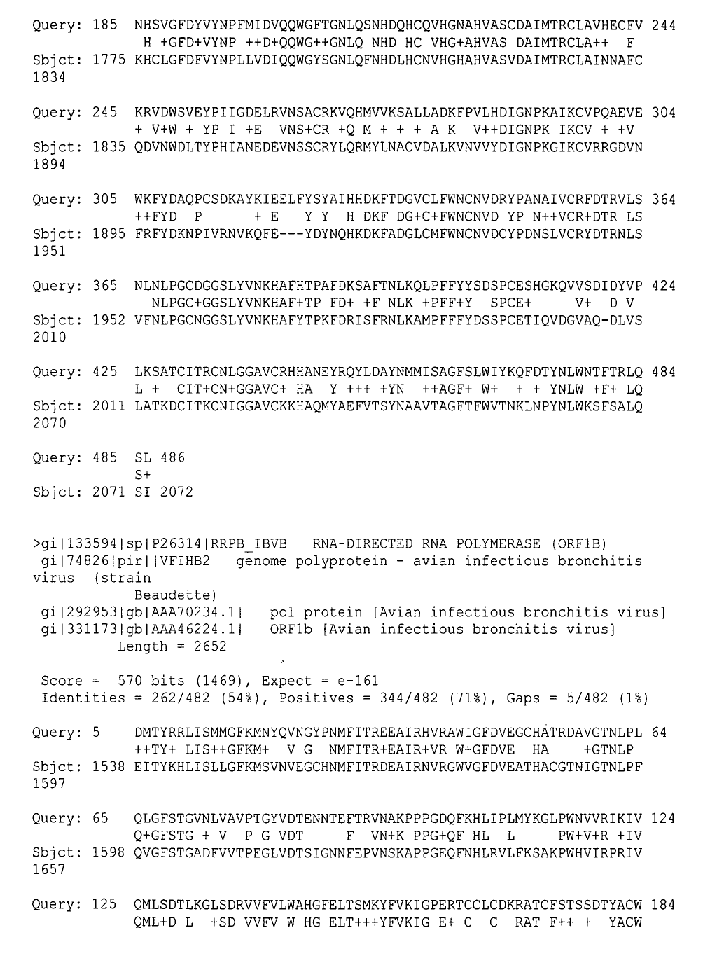

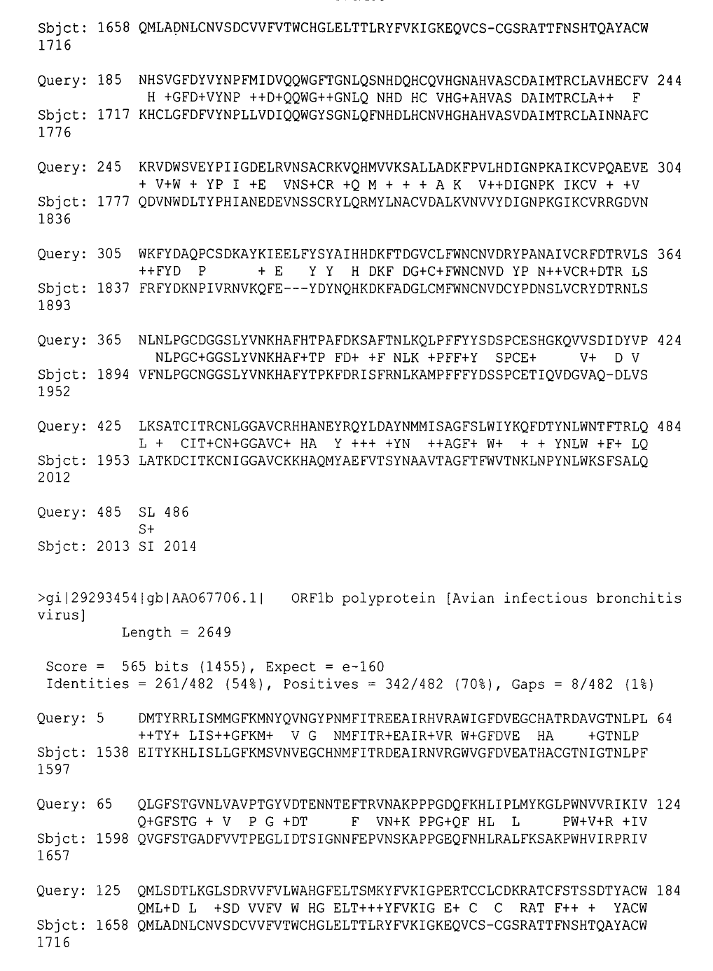

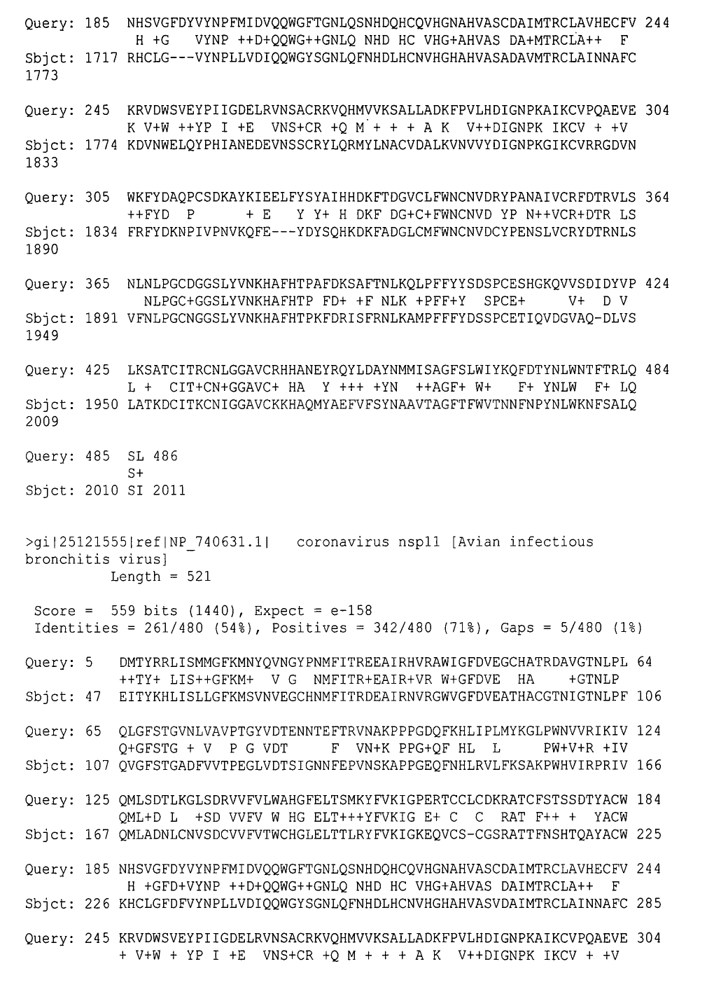

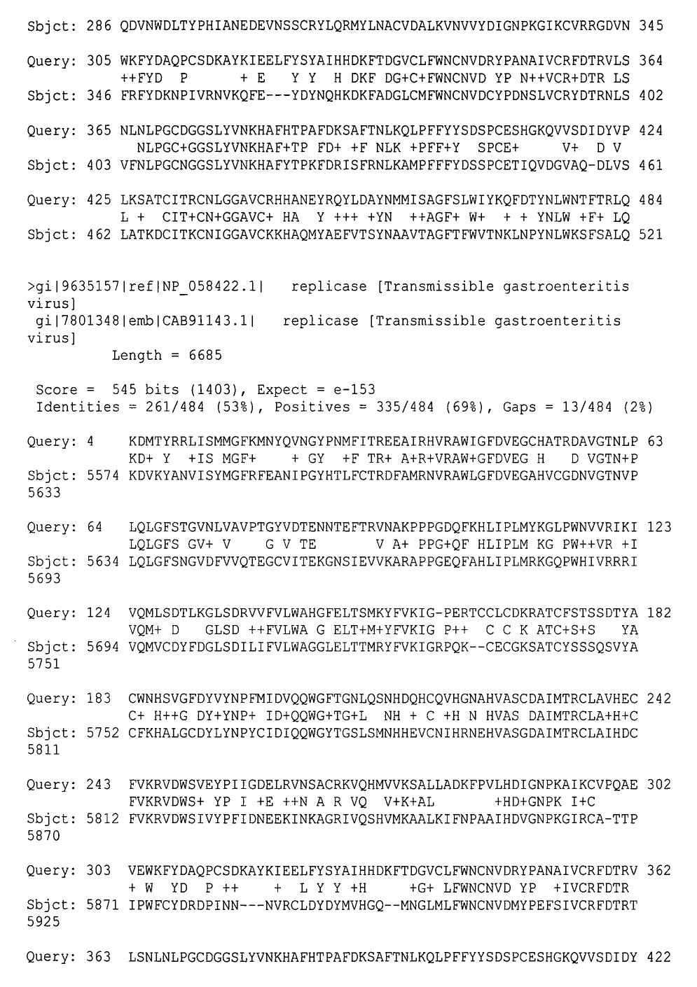

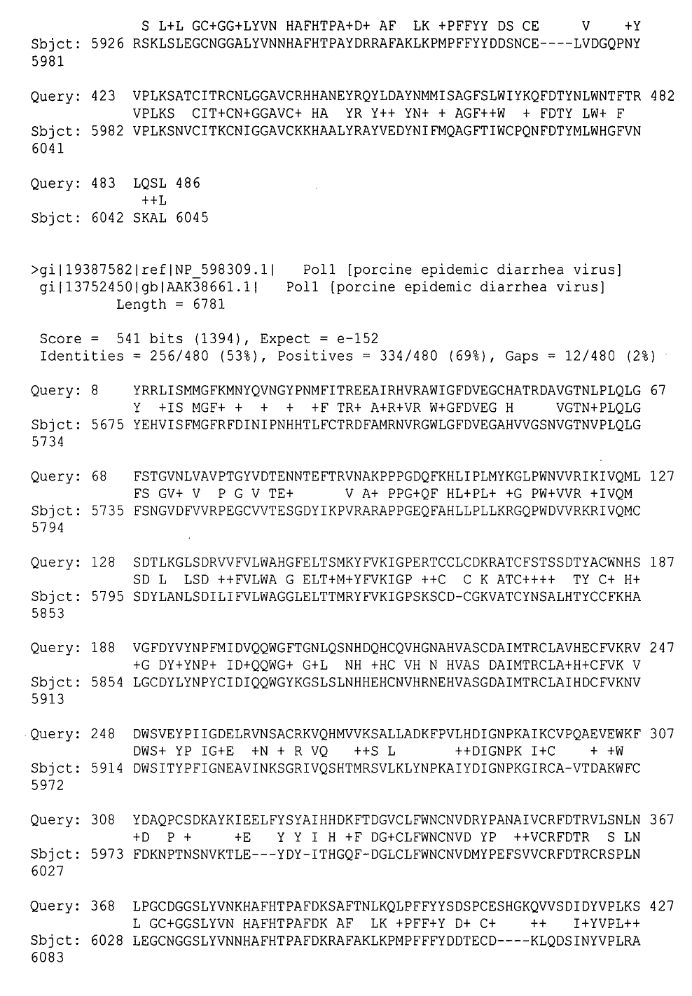

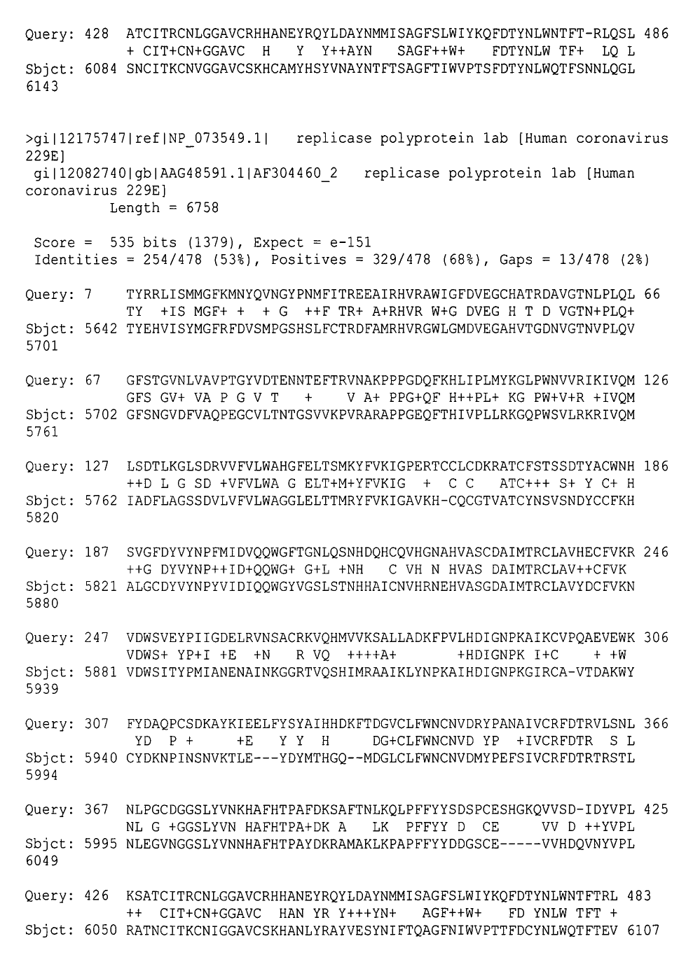

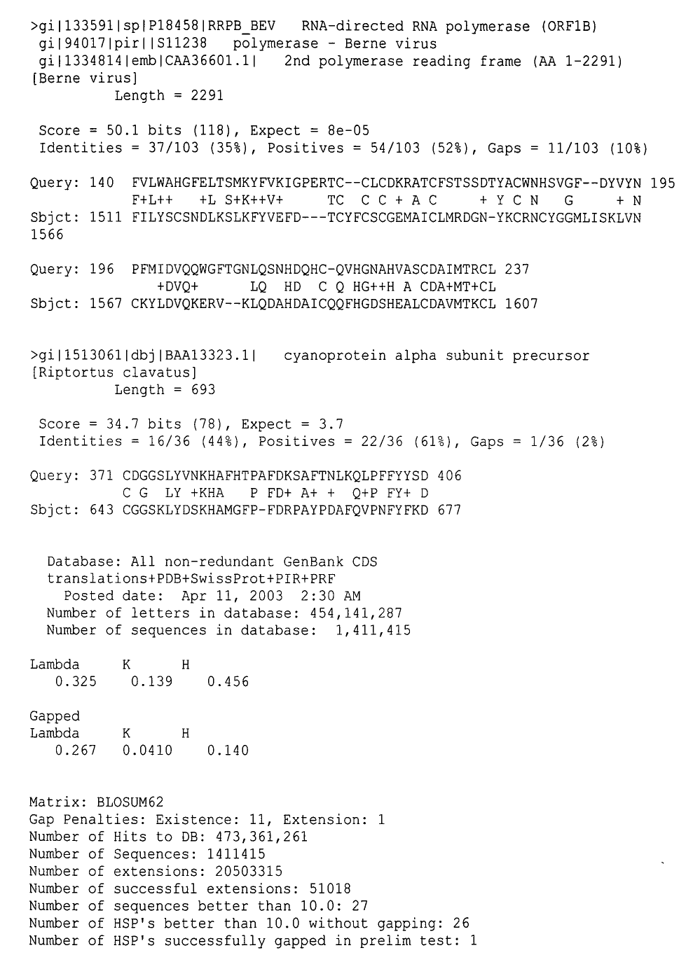

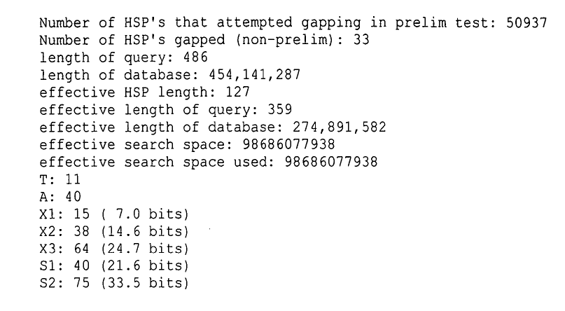

In one embodiment, the invention includes a polypeptide comprising the amino acid sequence selected from the group consisting of SEQ ID NO: 10210, SEQ ID NO: 10211 and SEQ ID NO: 10212. Partial results of a BLAST of SEQ ID NO: 10210 against GenBank is included in FIG. 124. These results indicate that SEQ ID NOS: 10210, 10211 and 10212 have functional similarities to a Coronavirus RNA polymerase, particularly the RNA polymerase of murine hepatitis virus, bovine coronavirus, and avian infectious bronchitis.

In one embodiment, the invention is directed to a polypeptide comprising a first amino acid sequence selected from the group consisting of SEQ ID NO: 10210, SEQ ID NO: 10211 and SEQ ID NO: 10212 and a second amino acid sequence from the C-terminus of a coronavirus ORF1ab sequence. Preferably, the second amino acid sequence is from a bovine coronavirus. One example of this embodiment is shown below as SEQ ID NO: 10216. Amino acids 1-481 of SEQ ID NO: 10216 are the first amino acid sequence of SEQ ID NO: 10210, and amino acids 482-1152 are the second amino acid sequence of the C-terminus of a bovine coronavirus orf1ab polyprotein (Gi 26008080) (NP_{—}150073.2) (SEQ ID NO: 10217).

Accordingly, the invention includes a polypeptide comprising SEQ ID NO: 10216. The invention further includes a polypeptide comprising a first amino acid sequence of SEQ ID NO: 10210 and a second amino acid sequence of SEQ ID NO: 10217. The invention further includes a polypeptide comprising a first amino acid sequence having greater than x % identity to SEQ ID NO: 10210 and a second amino acid sequence having greater than y % identity to SEQ ID NO: 10217, wherein x is greater than or equal to 85% (e.g., 86%, 87%, 88%, 89%, 90%, 91%, 92%, 93%, 94%, 95%, 96%, 97%, 98%, 99% or more) and wherein y is greater than or equal to 60% (e.g., 65%, 70%, 75%, 80%, 85%, 90%, 95%, 98%, 99% or more).

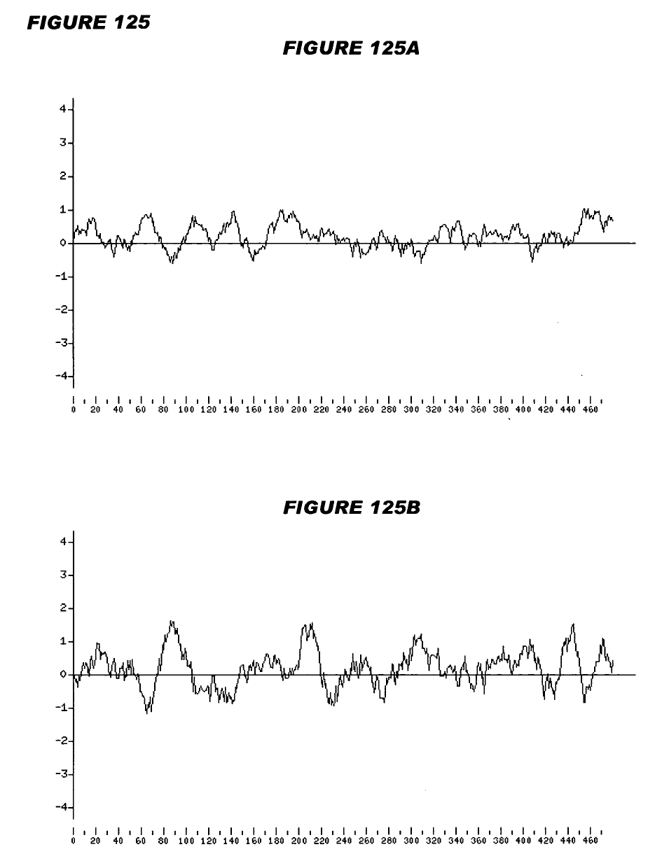

The invention also includes a polypeptide comprising a fragment of SEQ ID NO: 10210, wherein said fragment includes an epitope. Computer-predicted epitopes of SEQ ID NO: 10210, using a 17 mer window, are included in FIG. 125A (Hopp & Woods) and FIG. 125B (Kyte & Doolittle).

The amino acid sequence of SEQ ID NO: 10210 also contains two predicted glycosylation sites at amino acids 81-84 (NNTE; SEQ ID NO: 10218) and at 180-183 (NHSV; SEQ ID NO: 10219). Accordingly, the invention includes a polypeptide comprising a fragment of SEQ ID NO: 10210, wherein said fragment includes a glycosylation site. The invention further includes a polypeptide comprising a fragment of SEQ ID NO: 10210, wherein said fragment includes the Asn at position 81. Preferably, said Asn is glycosylated. The invention further includes a polypeptide comprising a fragment of SEQ ID NO: 10210, wherein said fragment includes the Asn at position 180. Preferably, said Asn is glycosylated.

In one embodiment, the invention includes a polypeptide comprising an amino acid sequence from within FIG. 120D and/or SEQ ID NO^{S}: 10150 to 10160 e.g. from SEQ ID NO^{S}: 10154, 10155, 10158 and 10160. Within SEQ ID NO: 10154 the following amino acid sequences starting with a Met and ending at a stop codon can be identified: SEQ ID NO^{S}: 10220 to 10227.

Accordingly, the invention includes a polypeptide comprising an amino acid sequence selected from the group consisting of SEQ ID NO: 10220, SEQ ID NO: 10221, SEQ ID NO: 10222, SEQ ID NO: 10223, SEQ ID NO: 10224, SEQ ID NO: 10225, SEQ ED NO: 10226 and SEQ ID NO: 10227, or a fragment thereof or an amino acid sequence having sequence identity thereto.

In one embodiment, the invention includes a polypeptide comprising the amino acid sequence within FIG. 120E e.g. from SEQ ID NO^{S}: 10161 to 10182, and in particular SEQ ID NOS: 10171 and 10176. Within SEQ ID NO^{S}: 10171 and 10176 the following amino acid sequences starting with a Met and ending at a stop codon can be identified: SEQ ID NO: 10228 and SEQ ID NO: 10229.

Accordingly, the invention includes a polypeptide comprising an amino acid sequence selected from the group consisting of SEQ ID NO: 10228 and SEQ ID NO: 10229, or a fragment thereof or an amino acid sequence having sequence identity thereto.

In one embodiment, the invention includes a polypeptide comprising an amino acid sequence from FIG. 120F e.g. SEQ ID NO^{S}: 10183 to 10209. Within FIG. 120F the following amino acid sequence starting with a Met and ending at a stop codon can be identified: SEQ ID NO: 10187. Accordingly, the invention includes a polypeptide comprising an amino acid sequence of SEQ ID NO: 10187, or a fragment thereof or an amino acid sequence having sequence identity thereto.

In one embodiment, the polynucleotides of the invention do not include one of the following primers, disclosed at http://content.nejm.org/cgi/reprint/NEJMoa030781v2.pdf.

| | |
|---|---|
| 5′GGGTTGGGACTATCCTAAGTGTGA3′ | (SEQ ID NO: 10230) |
| 5′TAACACACAACICCATCATCA3′ | (SEQ ID NO: 10231) |
| 5′CTAACATGCTTAGGATAATGG3′ | (SEQ ID NO: 10232) |
| 5′GCCTCTCTTGTTCTTGCTCGC3′ | (SEQ ID NO: 10233) |
| 5′CAGGTAAGCGTAAAACTCATC3′ | (SEQ ID NO: 10234) |

The invention also includes polynucleotide sequences which can be used as probes for diagnostic reagents, kits (comprising such reagents) and methods which can be used to diagnose or identify the presence or absence of a SARS virus in a biological sample. The invention includes the polynucleotide primers identified in Table 31 (SEQ ID NO^{S}: 10235 to 10258), the forward primers SEQ ID NO^{S}: 10259 to 10281 and the reverse primers SEQ ID NO^{S}: 10282 to 10298. The invention further includes polynucleotide sequences which are complementary to any one of these primer sequences disclosed herein.

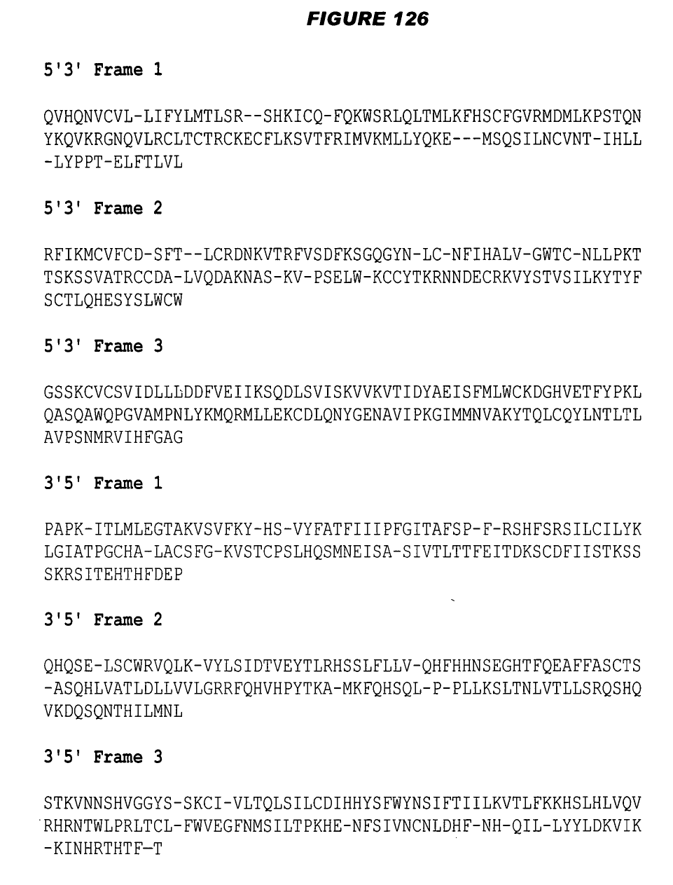

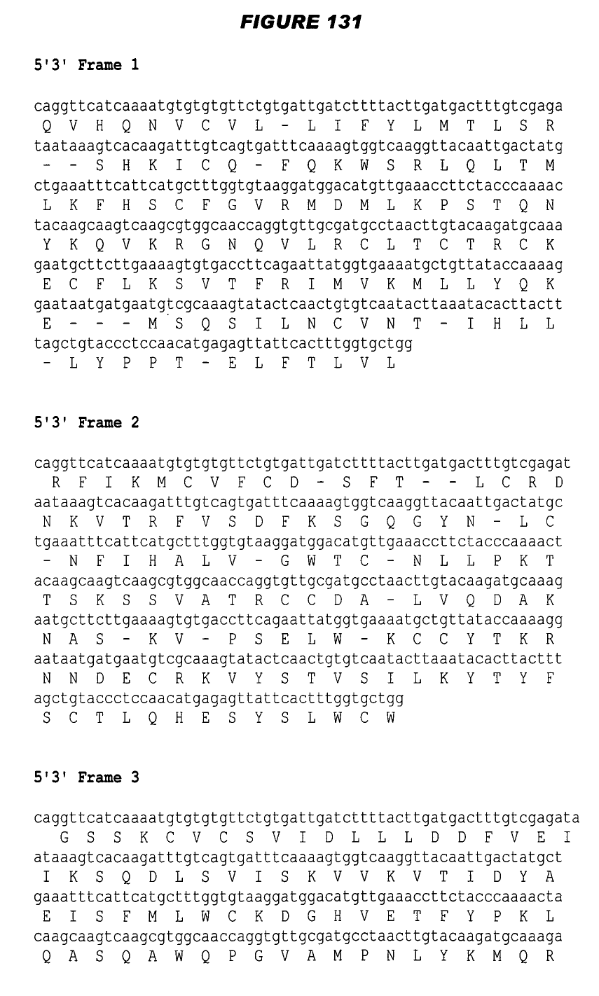

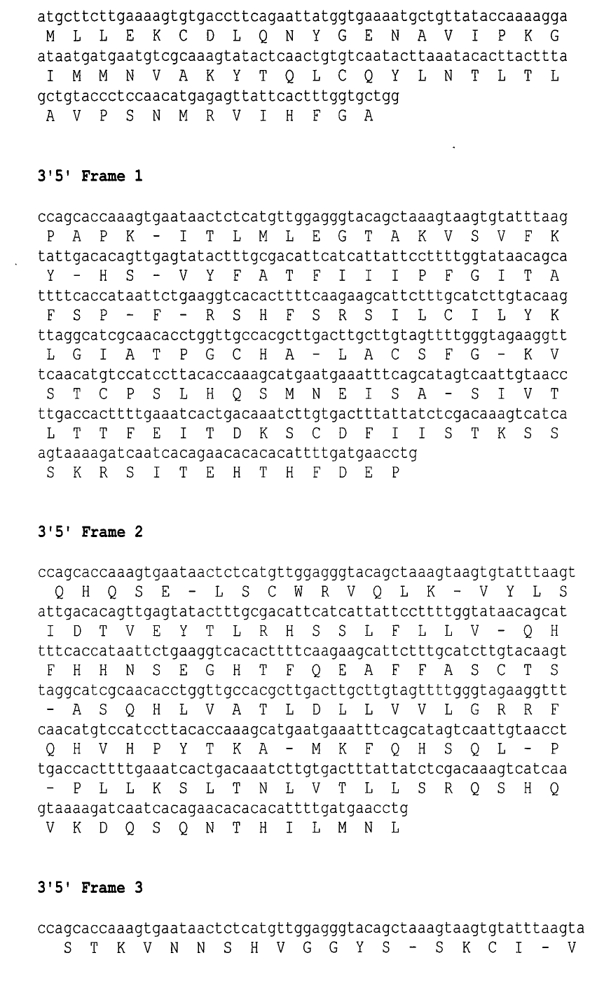

The invention provides a SARS polynucleotide sequence SEQ ID NO: 10299. All six reading frames of this sequence are included in FIG. 126 (See also FIG. 131). The constituent amino acid sequences from FIG. 126, having at least 4 amino acids, are listed as SEQ ID NOS: 10300 to 10337.

Accordingly, the invention includes a polynucleotide sequence comprising SEQ ID NO: 10299. It also provides polynucleotide sequences having sequence identity to SEQ ID NO: 10299. The invention also provides for polynucleotide sequences comprising fragments of SEQ ID NO: 10299. In one embodiment, the polynucleotide fragment does not consist entirely of a known polynucleotide sequence of a SARS virus or a known polynucleotide sequence of a coronavirus.

The invention includes an amino acid sequence encoded by the polynucleotide sequence of SEQ ID NO: 10299, including the amino acid sequences shown in FIG. 126, and the amino acid sequences selected from the group consisting of SEQ ID NO^{S}: 10300 to 10337. Preferably, the amino acid sequence comprises SEQ ID NO: 10316.

The invention also provides amino acid sequences having sequence identity to an amino acid sequence encoded by SEQ ID NO: 10299. The invention provides amino acid sequences having identity to an amino acid sequence selected from the group consisting of SEQ ID NO^{S}: 10300 to 10337.

The invention also provides fragments of amino acid sequences encoded by SEQ ID NO: 10299. The invention also provides fragments of amino acid sequences selected from the group consisting of SEQ ID NO^{S}: 10300 to 10337. In one embodiment, the fragment does not consist entirely of a known amino acid sequence of a SARS virus or a known amino acid sequence of a coronavirus.

In one embodiment, the invention comprises an amino acid sequence comprising SEQ ID NO: 10316. Encoded open reading frames within SEQ ID NO: 10316 include SEQ ID NO: 10338 and SEQ ID NO: 10339.

In one embodiment, the invention comprises an amino acid sequence comprising a sequence from within the 5′3′ Frame 1 translation of SEQ ID NO: 10299. The following encoded open reading frame is found within this translation: SEQ ID NO: 10340.

In one embodiment, the invention comprises an amino acid sequence comprising a sequence from within the 3′5′ Frame 1 translation of SEQ ID NO: 10299. An encoded open reading frame within this translation is SEQ ID NO: 10341.

In one embodiment, the invention comprises an amino acid sequence comprising a sequence from within the 3′5′ Frame 2 translation of SEQ ID NO: 10299. An encoded open reading frame within this translation is SEQ ID NO: 10342.

The invention includes a polypeptide comprising an amino acid sequence selected from the group consisting of SEQ ID NO: 10338, SEQ ID NO: 10339, SEQ ID NO: 10340, SEQ ID NO: 10341 and SEQ ID NO: 10342. The invention includes a polypeptide having sequence identity to an amino acid sequence selected from the group consisting of SEQ ID NO: 10338, SEQ ID NO: 10339, SEQ ID NO: 10340, SEQ ID NO: 10341 and SEQ ID NO: 10342. The invention includes a fragment of a polypeptide comprising an amino acid sequence elected from the group consisting of SEQ ID NO: 10338, SEQ ID NO: 10339, SEQ ID NO: 10340, SEQ ID NO: 10341 and SEQ ID NO: 10342. In one embodiment, the fragment does not consist entirely of a known SARS virus amino acid sequence or of a known coronavirus amino acid sequence.

In one embodiment, SEQ ID NOS: 10338-10342 are used in fusion proteins. Accordingly, the start codon methionines may be removed. The invention comprises a amino acid sequence selected from the group consisting of SEQ ID NO: 10343, SEQ ID NO: 10344, SEQ ID NO: 10345, SEQ ID NO: 10346 and SEQ ID NO: 10347.

In one embodiment, the invention comprises an amino acid sequence selected from the group consisting of SEQ ID NO: 10338 and SEQ ID NO: 10339. Partial BLAST results of SEQ ID NO: 10338 against GenBank are given below:

```
>gi|133593|sp|P18457|RRPB_CVPFS RNA-DIRECTED RNA POLYMERASE (ORF1B)
  gi|93934|pir||A43489 RNA-directed RNA polymerase (EC 2.7.7.48) - porcine
transmissible gastroenteritis virus (fragment)
  gi|833161|emb|CAA37284.1| polymerase [Transmissible gastroenteritis
virus]
                    Length = 533
  Score = 131 bits (329), Expect = 3e−30
  Identities = 55/89 (61%), Positives = 69/89 (77%), Gaps = 1/89 (1%).
Query: 1 MLWCKDGHVETFYPKLQASQAWQPGVAMPNLYKMQRMLLEKCDLQNYGENAVIPKGIMMN 60
                      MLWC++ H++TFYP+LQ+++ W PG +MP LYK+QRM LE+C+L NYG +P GI N
Sbjct: 217 MLWCENSHIKTFYPQLQSAE-WNPGYSMPTLYKIQRMCLERCNLYNYGAQVKLPDGITTN 275
Query: 61 VAKYTQLCQYLNTLTLAVPSNMRVIHFGA 89
                      V KYTQLCQYLNT TL VP MRV+H GA
Sbjct: 276 VVKYTQLCQYLNTTTLCVPHKMRVLHLGA 304
```

These results indicate that SEQ ID NO: 10338 has functional similarities to an RNA-directed RNA polymerase of porcine transmissible gastroenteritis virus.

Partial BLAST results of SEQ ID NO: 10339 against GenBank are given below:

```
>gb|AAL57305.1|replicase [bovine coronavirus]
                    Length = 7094
  Score = 139 bits (351), Expect = 7e−33
  Identities = 64/108 (59%), Positives = 78/108 (72%)
Query: 1 MSVISKVVKVTIDYAEISFMLWCKDGHVETFYPKLQASQAWQPGVAMPNLYKMQRMLLEK 60
                          M+ +SKVV V +D+ + FMLWC D V TFYP+LQA+ W+PG +MP LYK +E+
Sbjct: 6760 LNCVSKVVNVNVDFKDFQFMLWCNDEKVMTFYPRLQAASDWKPGYSMPVLYKYLNSPMER 6819
Query: 61 CDLQNYGENAVIPKGIMMNVAKYTQLCQYLNTLTLAVPSNMRVIHFGA 108
                          L NYG+ +P G MMNVAKYTQLCQYLNT TLAVP NMRV+H GA
Sbjct: 6820 VSLWNYGKPVTLPTGCMMNVAKYTQLCQYLNTTTLAVPVNMRVLHLGA 6867
```

These results indicate that SEQ ID NO: 10339 has functional similarities to a replicase of bovine coronavirus.

The SARS virus may contain polymorphism at the Glu-20 residue of SEQ ID NO: 10338. The invention includes a polypeptide comprising an amino acid sequence having sequence identity to SEQ ID NO: 10338, wherein said polypeptide includes an amino acid sequence selected from the group consisting of ASQAW (SEQ ID NO: 10348) and ASRAW (SEQ ID NO: 10349). The invention includes a fragment of a polypeptide comprising SEQ ID NO: 10338, wherein said fragment includes an amino acid sequence selected from the group consisting of SEQ ID NO: 10348 and SEQ ID NO: 10349.

The SARS virus may contain polymorphism at the Ser-80 residue of SEQ ID NO: 10338. below. The invention includes a polypeptide comprising an amino acid sequence having sequence identity to SEQ ID NO: 10338, wherein said polypeptide includes an amino acid sequence selected from the group consisting of VPSNM (SEQ ID NO: 10350) and VPTNM (SEQ ID NO: 10351). The invention includes a fragment of a polypeptide comprising SEQ ID NO: 10338, wherein said fragment includes an amino acid sequence selected from the group consisting of SEQ ID NO: 10350 and SEQ ID NO: 10351.

The invention also includes polynucleotide sequences which can be used as probes for diagnostic reagents, kits (comprising such reagents) and methods which can be used to diagnose or identify the presence or absence of a SARS virus in a biological sample. The invention includes a polynucleotide sequence comprising one or more of the primer sequences identified in Table 32. The invention further includes polynucleotide sequence comprising the complement of one or more of the primer sequences identified in Table 32.

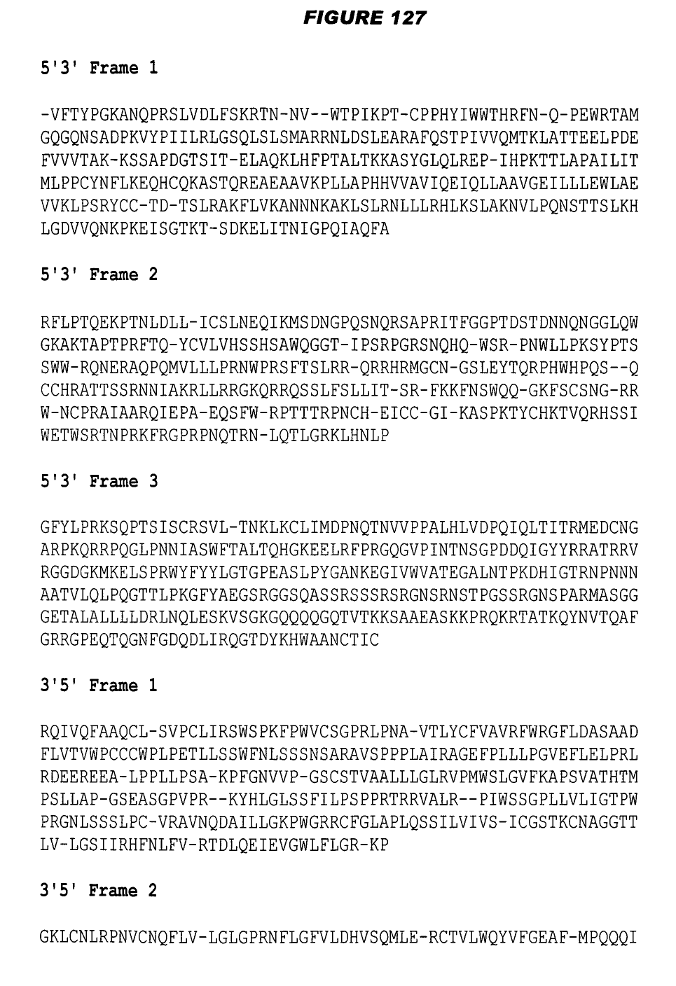

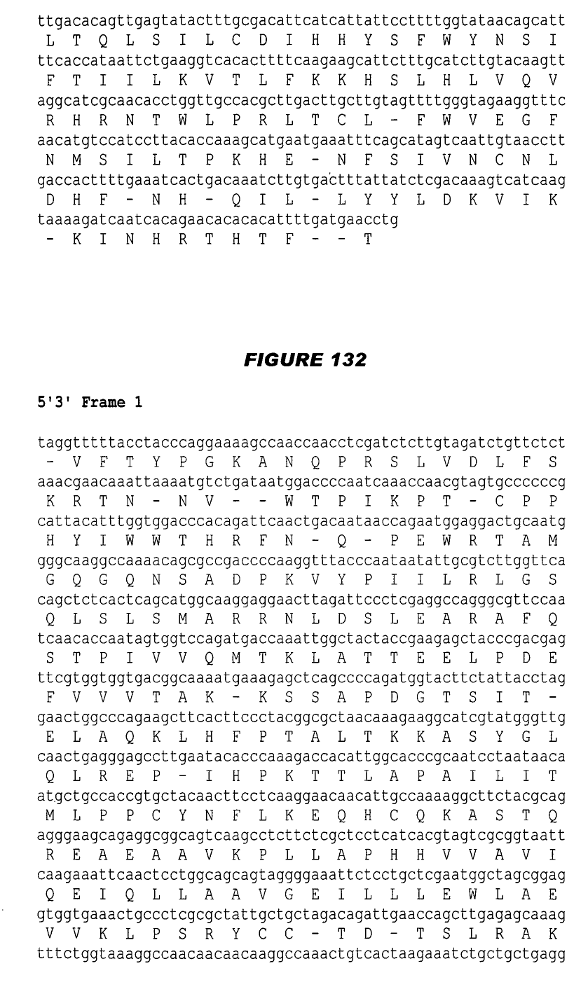

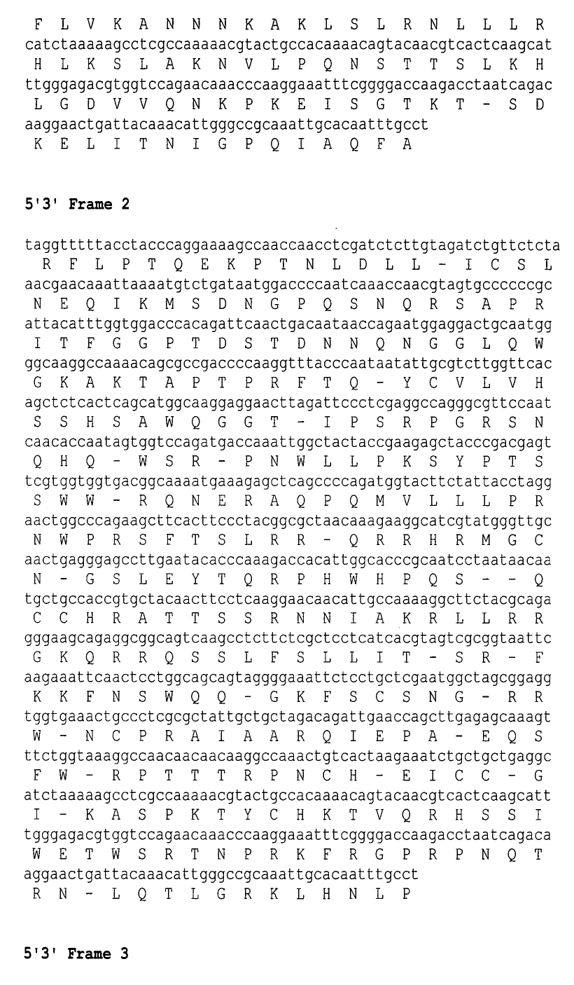

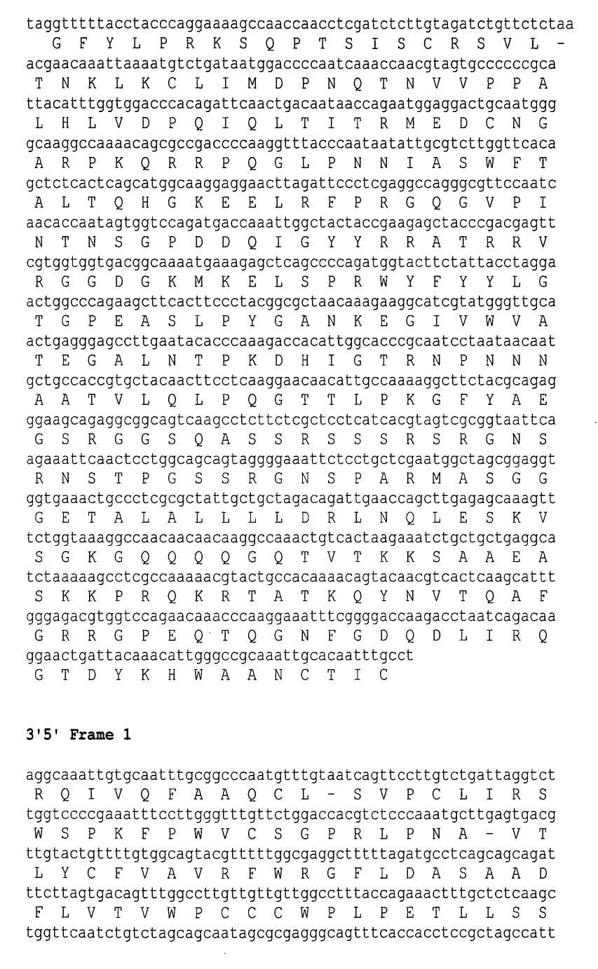

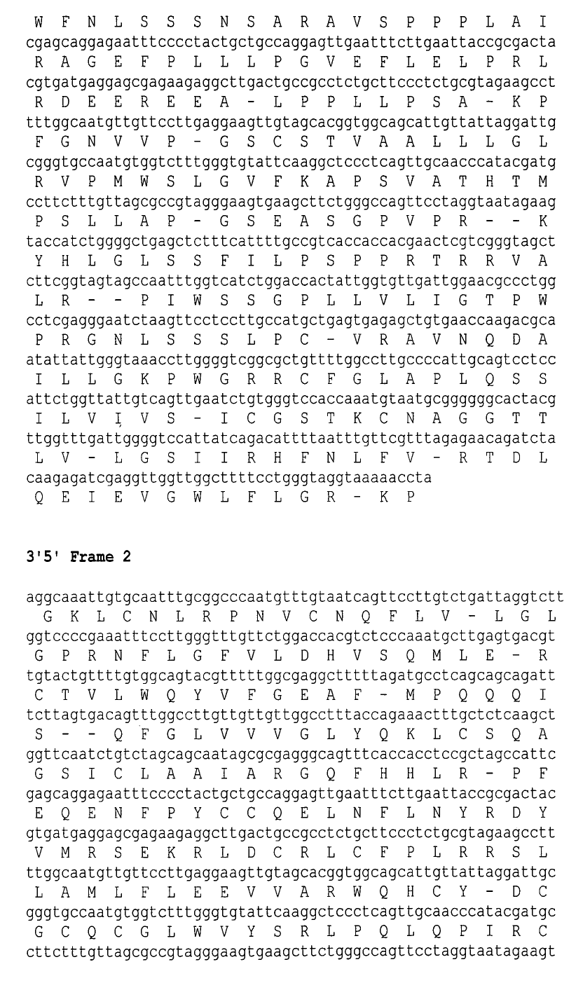

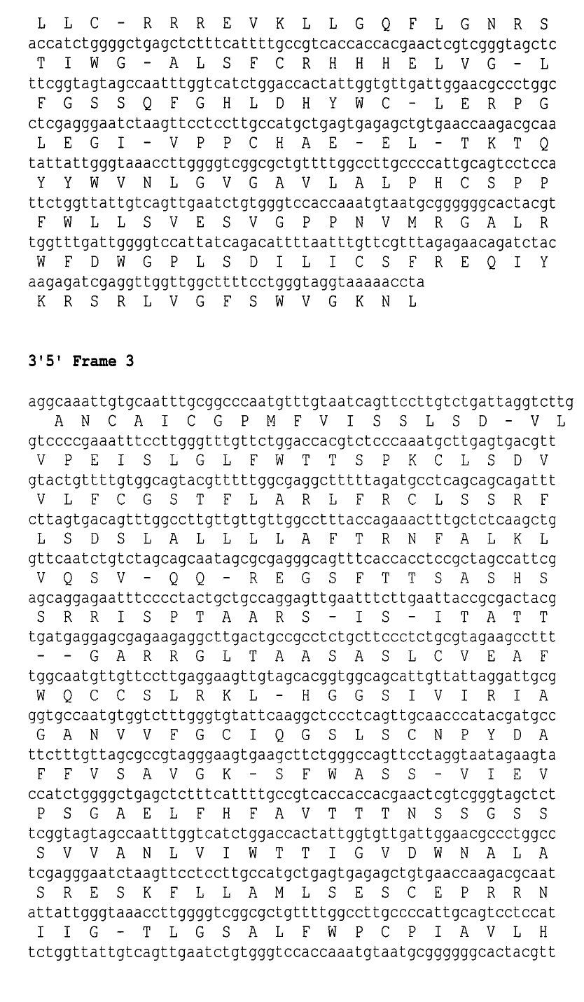

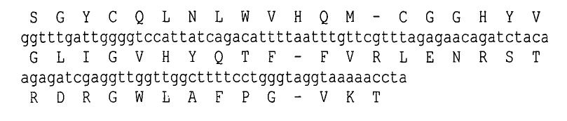

The invention provides a SARS polynucleotide sequence SEQ ID NO: 10505. All six reading frames of this sequence are shown in FIG. 127 (see also FIG. 132). The constituent amino acid sequences from FIG. 127, having at least 4 amino acids, are listed as SEQ ID NOS: 10506 to 10570.

The invention includes a polynucleotide sequence comprising SEQ ID NO: 10505. The invention also provides polynucleotide sequences having sequence identity to SEQ ID NO: 10505. The invention also provides for polynucleotide sequences comprising fragments of SEQ ID NO: 10505. In one embodiment, the polynucleotide fragment does not consist entirely of a known SARS virus polynucleotide sequence or of a known coronavirus polynucleotide sequence.

The invention includes an amino acid sequence encoded by the polynucleotide sequence of SEQ ID NO: 10505, including the amino acid sequences shown in FIG. 127, and particularly those selected from the group consisting of SEQ ID NO^{S}: 10506 to 10570. Preferably, the amino acid sequence comprises SEQ ID NO: 10532 and/or SEQ ID NO: 10533.

The invention also provides amino acid sequences having sequence identity to an amino acid sequence encoded by SEQ ID NO: 10505. The invention provides amino acid sequences having sequence identity to an amino acid sequence selected from the group consisting of the sequences shown in FIG. 127, and in particular SEQ ID NO^{S}: 10506 to 10570.

The invention also provides fragments of amino acid sequences encoded by SEQ ID NO: 10505. The invention also provides fragments of amino acid sequences selected from the group consisting of SEQ ID NO^{S}: 10506 to 10570. In one embodiment, the fragment does not consist entirely of a known amino acid sequence of a SARS virus or a known amino acid sequence of a coronavirus.

In one embodiment, the invention includes a polypeptide comprising an amino acid sequence from the 5′3′ Frame 3 of FIG. 127. Some encoded open reading frames within this translation are: SEQ ID NO: 10533; SEQ ID NO: 10571; SEQ ID NO: 10572; SEQ ID NO: 10573; SEQ ID NO: 10574.

The invention includes a polypeptide sequence comprising an amino acid sequence selected from the group consisting of SEQ ID NO: 10533, SEQ ID NO: 10571, SEQ ID NO: 10572, SEQ ID NO: 10573 and SEQ ID NO: 10574. The invention includes a polypeptide having sequence identity to the amino acid sequence selected from the group consisting of SEQ ID NO: 10533, SEQ ID NO: 10571, SEQ ID NO: 10572, SEQ ID NO: 10573 and SEQ ID NO: 10574. The invention includes a fragment of a polypeptide sequence comprising an amino acid sequence selected from the group consisting of SEQ ID NO: 10533, SEQ ID NO: 10571, SEQ ID NO: 10572, SEQ ID NO: 10573 and SEQ ID NO: 10574.

Partial BLAST results of SEQ ID NO: 10533 against GenBank are given below:

```
>gi|7739601|gb|AAF68926.1|AF207902_11 nucleocapsid protein [murine
hepatitis virus strain ML-11]
                    Length = 451
  Score = 147 bits (370), Expect = 3e−34
  Identities = 102/252 (40%), Positives = 137/252 (54%), Gaps = 18/252 (7%)
Query: 49 SWFTALTQHGK-EELRFPRGQGVPINTNSGPDDQIGYYRRATRR-VRGGDGKMKELSPRW 106
                      SWF+ +TQ K +E +F +GQGVPI + +Q GY+ R RR + DG+ K+L PRW
Sbjct: 63 SWFSGITQFQKGKEFQFAQGQGVPIASGIPASEQKGYWYRHNRRSFKTPDGQHKQLLPRW 122
Query: 107 YFYYLGTGPEASLPYGANKEGIVWVATEGALNTPKDHIGTRNPNNNAATVLQLPQGTTLP 166
                      YFYYLGTGP A YG + EG+VWVA++ A + R+P+++ A + GT LP
Sbjct: 123 YFYYLGTGPHAGAEYGDDIEGVVWVASQQADTKTTADVVERDPSSHEAIPTRFAPGTVLP 182
Query: 167 KGFYAEGSRGGSQASSRSSSRSRGNSRNSTPGSSRGNSPARMASGGGETALALLLLDRLN 226
                      +GFY EGS + AS S N SS PA +A L+L +L
Sbjct: 183 QGFYVEGSGRSAPASRSGSRSQSRGPNNRARSSSNQRQPASAVKPDMAEEIAALVLAKLG 242
Query: 227 QLESKVSGKGQQQQGQTVTKKSAAEASK----KPRQKRTATKQYNVTQAFGRRGPEQTQG 282
                      + GQ +Q VTK+SA E + KPRQKRT KQ V Q FG+RGP Q
Sbjct: 243 K------DAGQPKQ---VTKQSAKEVRQKILTKPRQKRTPNKQCPVQQCFGKRGPNQ--- 290
Query: 283 NFGDQDLIRQGT 294
                      NFG ++++ GT
Sbjct: 291 NFGGSEMLKLGT 302
>gi|3132999|gb|AAC16422.1| nucleocapsid protein [murine hepatitis virus
strain 2]
                    Length = 451
  Score = 147 bits (370), Expect = 3e−34
  Identities = 102/252 (40%), Positives = 137/252 (54%), Gaps = 18/252 (7%)
Query: 49 SWFTALTQHGK-EELRFPRGQGVPINTNSGPDDQIGYYRRATRR-VRGGDGKMKELSPRW 106
                      SWF+ +TQ K +E +F +GQGVPI + +Q GY+ R RR + DG+ K+L PRW
Sbjct: 63 SWFSGITQFQKGKEFQFAQGQGVPIASGIPASEQKGYWYRHNRRSFKTPDGQHKQLLPRW 122
Query: 107 YFYYLGTGPEASLPYGANKEGIVWVATEGALNTPKDHIGTRNPNNNAATVLQLPQGTTLP 166
                      YFYYLGTGP A YG + EG+VWVA++ A + R+P+++ A + GT LP
Sbjct: 123 YFYYLGTGPHAGAEYGDDIEGVVWVASQQADTKTTADVVERDPSSHEAIPTKFAPGTVLP 182
Query: 167 KGFYAEGSRGGSQASSRSSSRSRGNSRNSTPGSSRGNSPARMASGGGETALALLLLDRLN 226
                      +GFY EGS + AS S N SS PA +A L+L +L
Sbjct: 183 QGFYVEGSGKSAPASRSGSRSQSRGPNNRARSSSNQRQPASAVKPDMAEEIAALVLAKLG 242
Query: 227 QLESKVSGKGQQQQGQTVTKKSAAEASK----KPRQKRTATKQYNVTQAFGRRGPEQTQG 282
                      + GQ +Q VTK+SA E + KPRQKRT KQ V Q FG+RGP Q
Sbjct: 243 K------DAGQPKQ---VTKQSAKEVRQKILTKPRQKRTPNKQCPVQQCFGKRGPNQ--- 290
Query: 283 NFGDQDLIRQGT 294
                      NFG ++++ GT
Sbjct: 291 NFGGSEMLKLGT 302
>gi|127877|sp|P03417|NCAP_CVMJH Nucleocapsid protein
  gi|174859|pir||VHIHMJ nucleocapsid protein - murine hepatitis virus
(strain JHM)
  gi|58973|emb|CAA25497.1| nucleocapsid protein [Murine hepatitis virus]
                    Length = 455
  Score = 146 bits (369), Expect = 4e−34
  Identities = 110/254 (43%), Positives = 142/254 (55%), Gaps = 22/254 (8%)
Query: 49 SWFTALTQHGK-EELRFPRGQGVPINTNSGPDDQIGYYRRATRR-VRGGDGKMKELSPRW 106
                      SWF+ +TQ K +E +F +GQGVPI Q GY+ R RR + DG+ K+L PRW
Sbjct: 67 SWFSGITQFQKGKEFQFAQGQGVPIANGIPASQQKGYWYRHNRRSFKTPDGQQKQLLPRW 126
Query: 107 YFYYLGTGPEASLPYGANKEGIVWVATEGALNTPKDHIGTRNPNNNAATVLQLPQGTTLP 166
                      YFYYLGTGP A YG + EG+VWVA++ A I R+P+++ A + GT LP
Sbjct: 127 YFYYLGTGPYAGAEYGDDIEGVVWVASQQAETRTSADIVERDPSSHEAIPTRFAPGTVLP 186
Query: 167 KGFYAEGSRGGSQASSRSSSR--SRGNSRNSTPGSSRGNSPARMASGGGETALALLLLDR 224
                      +GFY EGS G S +SRS SR SRG N SS PA +A L+L +
Sbjct: 187 QGFYVEGS-GRSAPASRSGSRPQSRG-PNNRARSSSNQRQPASTVKPDMAEEIAALVLAK 244
Query: 225 LNQLESKVSGKGQQQQGQTVTKKSAAEASK----KPRQKRTATKQYNVTQAFGRRGPEQT 280
                      L + GQ +Q VTK+SA E + KPRQKRT KQ V Q FG+RGP Q
Sbjct: 245 LGK------DAGQPKQ---VTKQSAKEVRQKILNKPRQKRTPNKQCPVQQCFGKRGPNQ- 294
Query: 281 QGNFGDQDLIRQGT 294
                          NFG ++++ GT
Sbjct: 295 --NFGGPEMLKLGT 306
>gi|6625766|gb|AAF19389.1|AF201929_7 nucleocapsid protein [murine
hepatitis virus strain 2]
  gi|7769348|gb|AAF69338.1|AF208066_11 nucleocapsid protein [murine
hepatitis virus]
                    Length = 451
  Score = 146 bits (368), Expect = 5e−34
  Identities = 102/252 (40%), Positives = 137/252 (54%), Gaps = 18/252 (7%)
Query: 49 SWFTALTQHGK-EELRFPRGQGVPINTNSGPDDQIGYYRRATRR-VRGGDGKMKELSPRW 106
                      SWF+ +TQ K +E +F +GQGVPI + +Q GY+ R RR + DG+ K+L PRW
Sbjct: 63 SWFSGITQFQKGKEFQFAQGQGVPIASGIPASEQKGYWYRHNRRSFKTPDGQHKQLLPRW 122
Query: 107 YFYYLGTGPEASLPYGANKEGIVWVATEGALNTPKDHIGTRNPNNNAATVLQLPQGTTLP 166
                      YFYYLGTGP A YG + EG+VWVA++ A + R+P+++ A + GT LP
Sbjct: 123 YFYYLGTGPHAGAEYGDDIEGVVWVASQQADTKTTADVVERDPSSHEAIPTRFAPGTVLP 182
Query: 167 KGFYAEGSRGGSQASSRSSSRSRGNSRNSTPGSSRGNSPARMASGGGETALALLLLDRLN 226
                      +GFY EGS + AS S N SS PA +A L+L +L
Sbjct: 183 QGFYVEGSGRSAPASRSGSRSQSRGPNNRARSSSNQRQPASAVKPDMAEEIAALVLAKLG 242
Query: 227 QLESKVSGKGQQQQGQTVTKKSAAEASK----KPRQKRTATKQYNVTQAFGRRGPEQTQG 282
                      + GQ +Q VTK+SA E + KPRQKRT KQ V Q FG+RGP Q
Sbjct: 243 K------DAGQPKQ---VTKQSAKEVRQKILTKPRQKRTPNKQCPVQQCFGKRGPNQ--- 290
Query: 283 NFGDQDLIRQGT 294
                      NFG ++++ GT
Sbjct: 291 NFGGSEMLKLGT 302
>gi|21734854|gb|AAM77005.1|AF481863_7 phosphorylated nucleocapsid protein
N [porcine hemagglutinating encephalomyelitis virus]
                    Length = 449
  Score = 145 bits (366), Expect = 8e−34
  Identities = 107/253 (42%), Positives = 145/253 (57%), Gaps = 18/253 (7%)
Query: 49 SWFTALTQHGK-EELRFPRGQGVPINTNSGPDDQIGYYRRATRR-VRGGDGKMKELSPRW 106
                      SWF+ +TQ K +E F GQGVPI + GY+ R RR + DG ++L PRW
Sbjct: 64 SWFSGITQFQKGKEFEFAEGQGVPIAPGVPATEAKGYWYRHNRRSFKTADGNQRQLLPRW 123
Query: 107 YFYYLGTGPEASLPYGANKEGIVWVATEGA-LNTPKDHIGTRNPNNNAATVLQLPQGTTL 165
                      YFYYLGTGP A YG + +G+ WVA+ A +NTP D I R+P+++ A + P GT L
Sbjct: 124 YFYYLGTGPHAKHQYGTDIDGVFWVASNQADINTPAD-IVDRDPSSDEAIPTRFPPGTVL 182
Query: 166 PKGFYAEGSRGGSQASSRSSSRSRGNSRNSTPGSSRGNSPARMASGGGETALALLLLDRL 225
                      P+G+Y EGS G S +SRS+SR+ N S SR NS R ++ G +A D++
Sbjct: 183 PQGYYIEGS-GRSAPNSRSTSRA-PNRAPSAGSRSRANSGNRTSTPGVTPDMA----DQI 236
Query: 226 NQLESKVSGKGQQQQGQTVTKKSAAEASK----KPRQKRTATKQYNVTQAFGRRGPEQTQ 281
                          L GK + Q VTK++A E + KPRQKR+ KQ V Q FG+RGP Q
Sbjct: 237 ASLVLAKLGK-DATKPQQVTKQTAKEVRQKILNKPRQKRSPNKQCTVQQCFGKRGPNQ-- 293
Query: 282 GNFGDQDLIRQGT 294
                        NFG ++++ GT
Sbjct: 294 -NFGGGEMLKLGT 305
>gi|23295765|gb|AAL80036.1| nucleocapsid protein [porcine
hemagglutinating encephalomyelitis virus]
                    Length = 449
  Score = 145 bits (365), Expect = 1e−33
  Identities = 107/253 (42%), Positives = 145/253 (57%), Gaps = 18/253 (7%)
Query: 49 SWFTALTQHGK-EELRFPRGQGVPINTNSGPDDQIGYYRRATRR-VRGGDGKMKELSPRW 106
                      SWF+ +TQ K +E F GQGVPI + GY+ R RR + DG ++L PRW
Sbjct: 64 SWFSGITQFQKGKEFEFAEGQGVPIAPGVPSTEAKGYWYRHNRRSFKTADGNQRQLLPRW 123
Query: 107 YFYYLGTGPEASLPYGANKEGIVWVATEGA-LNTPKDHIGTRNPNNNAATVLQLPQGTTL 165
                      YFYYLGTGP A YG + +G+ WVA+ A +NTP D I R+P+++ A + P GT L
Sbjct: 124 YFYYLGTGPHAKDQYGTDIDGVFWVASNQADINTPAD-IVDRDPSSDEAIPTRFPPGTVL 182
Query: 166 PKGFYAEGSRGGSQASSRSSSRSRGNSRNSTPGSSRGNSPARMASGGGETALALLLLDRL 225
                      P+G+Y EGS G S +SRS+SR+ N S SR NS R ++ G +A D++
Sbjct: 183 PQGYYIEGS-GRSAPNSRSTSRA-PNRAPSAGSRSRANSGNRTSTPGVTPDMA----DQI 236
Query: 226 NQLESKVSGKGQQQQGQTVTKKSAAEASK----KPRQKRTATKQYNVTQAFGRRGPEQTQ 281
                          L GK + Q VTK++A E + KPRQKR+ KQ V Q FG+RGP Q
Sbjct: 237 ASLVLAKLGK-DATKPQQVTKQTAKEVRQKILNKPRQKRSPNKQCTVQQCFGKRGPNQ-- 293
Query: 282 GNFGDQDLIRQGT 294
                        NFG ++++ GT
Sbjct: 294 -NFGGGEMLKLGT 305
```

These results indicate that SEQ ID NO: 10533 has functional similarities to a coronavirus nucleocapsid protein.

In one embodiment, the invention comprises an amino acid sequence from the 5′3′ Frame 1 of FIG. 127 e.g. SEQ ID NO^{S}: 10506-10514. Some encoded open reading frames within this region are SEQ ID NO^{S}: 10575 to 10578.

Accordingly, the invention includes a polypeptide comprising the amino acid sequence selected from the group consisting of SEQ ID NO: 10575, SEQ ID NO: 10576, SEQ ID NO: 10577 and SEQ ID NO: 10578. The invention includes a polypeptide comprising an amino acid sequence having sequence identity to a sequence selected from the group consisting of SEQ ID NO: 10097, SEQ ID NO: 10576, SEQ ID NO: 10577 and SEQ ID NO: 10578. The invention includes a fragment of a polypeptide comprising an amino acid sequence selected from the group consisting of SEQ ID NO: 10097, SEQ ID NO: 10576, SEQ ID NO: 10577 and SEQ ID NO: 10578.

In one embodiment, the invention includes a polypeptide comprising an amino acid sequence from the 3′5′ Frame 2 of FIG. 127 e.g. SEQ ID NO^{S}: 10547-10559. An open reading frame within this region is SEQ ID NO: 10579.

The invention includes a polypeptide comprising an amino acid sequence of SEQ ID NO: 10579. The invention includes a polypeptide comprising an amino acid sequence having sequence identity to SEQ ID NO: 10579. The invention includes a fragment of a polypeptide comprising an amino acid sequence of SEQ ID NO: 10579.

The invention also includes polynucleotide sequences which can be used as probes for diagnostic reagents, kits (comprising such reagents) and methods which can be used to diagnose or identify the presence or absence of a SARS virus in a biological sample. The invention includes a polynucleotide sequence comprising one or more of the primer sequences identified in Table 33. The invention further includes polynucleotide sequence comprising the complement of one or more of the primer sequences identified in Table 33.

The invention includes a polynucleotide sequence comprising SEQ ID NO: 11323. A polypeptide encoded by SEQ ID NO: 11323 is SEQ ID NO: 11324.

The invention includes a polypeptide comprising SEQ ID NO: 11324, sequence having sequence identity to SEQ ID NO: 11324 and fragments of SEQ ID NO: 11324. The invention includes a fragment of SEQ ID NO: 11324, wherein said polypeptide fragment begins with a Methionine.

Accordingly, the invention includes a polynucleotide sequence comprising SEQ ID NO: 11323. It also provides polynucleotide sequences having sequence identity to SEQ ID NO: 11323. The invention also provides for polynucleotide sequences comprising fragments of SEQ ID NO: 11323. In one embodiment, the polynucleotide fragment does not consist entirely of a known SARS polynucleotide sequence or a known coronavirus polynucleotide sequence.

The invention includes an amino acid sequence encoded by the polynucleotide sequence SEQ ID NO: 11323, including the amino acid sequence of SEQ ID NO: 11324.

The invention also provides amino acid sequences having sequence identity to an amino acid sequence encoded by SEQ ID NO: 11323. The invention provides amino acid sequences having sequence identity to SEQ ID NO: 11324.

The invention provides fragments of amino acid sequences encoded by SEQ ID NO: 11323. The invention also provides fragments of amino acid sequences of SEQ ID NO: 11324. In one embodiment, the fragment does not consist entirely of a known SARS amino acid sequence or a known coronavirus amino acid sequence.

The invention also includes polynucleotide sequences which can be used as probes for diagnostic reagents, kits (comprising such reagents) and methods which can be used to diagnose or identify the presence or absence of a SARS virus in a biological sample. The invention includes a polynucleotide sequence comprising one or more of the primer sequences identified as SEQ ID NO_{S}: 11325-11440 (left part) and SEQ ID NO^{S}: 11441-11551 (right part). The invention further includes polynucleotide sequence comprising the complement of one or more of the primer sequences identified as SEQ ID NO^{S}: 11325-11551.

The invention includes a polypeptide comprising SEQ ID NO: 11552. The SARS virus contains polymorphism at the Isoleucine residue Ile-324. The invention includes a polypeptide comprising an amino acid sequence having sequence identity to SEQ ID NO: 11552, wherein said polypeptide includes an amino acid sequence selected from the group consisting of YSYAI (SEQ ID NO: 11553), SYAIH (SEQ ID NO: 11554), YAIHH (SEQ ID NO: 11555), IHHDK (SEQ ID NO: 11556), SYAI (SEQ ID NO: 11557), YAIH (SEQ ID NO: 11558), AIHH (SEQ ID NO: 11559), IHHD (SEQ ID NO: 11560), YAI, AIH, and IHH. The invention includes a fragment of a polypeptide comprising SEQ ID NO: 11552, wherein said fragment includes an amino acid sequence selected from the group consisting of YSYAI (SEQ ID NO: 11553), SYAIH (SEQ ID NO: 11554), YAIHH (SEQ ID NO: 11555), IHHDK (SEQ ID NO: 11556), SYAI (SEQ ID NO: 11557), YAIH (SEQ ID NO: 11558), AIHH (SEQ ID NO: 11559), IHHD (SEQ ID NO: 11560), YAI, AIH, and IHH.

The invention includes a polypeptide comprising an amino acid sequence selected from the group consisting of SEQ ID NO: 11561 and SEQ ID NO: 11562. The invention includes a fragment of a polypeptide comprising an amino acid sequence selected from the group consisting of SEQ ID NO: 11561 and SEQ ID NO: 11562.

The invention includes a diagnostic kit comprising a polypeptide comprising at least one of the amino acid sequences selected from the group consisting of SEQ ID NO^{S}: 11561 and 11562. The invention includes a diagnostic kit comprising a polynucleotide sequence encoding a polypeptide comprising at least one of the amino acid sequences selected from the group consisting of SEQ ID NO^{S}: 11561 and 11562. The invention includes an immunogenic composition comprising a polypeptide comprising at least one of the amino acid sequences selected from the group consisting of SEQ ID NO^{S}: 11561 and 11562. The invention includes an antibody which recognizes a polypeptide comprising at least one of the amino acid sequences selected from the group consisting of SEQ ID NO^{S}: 11561 and 11562.

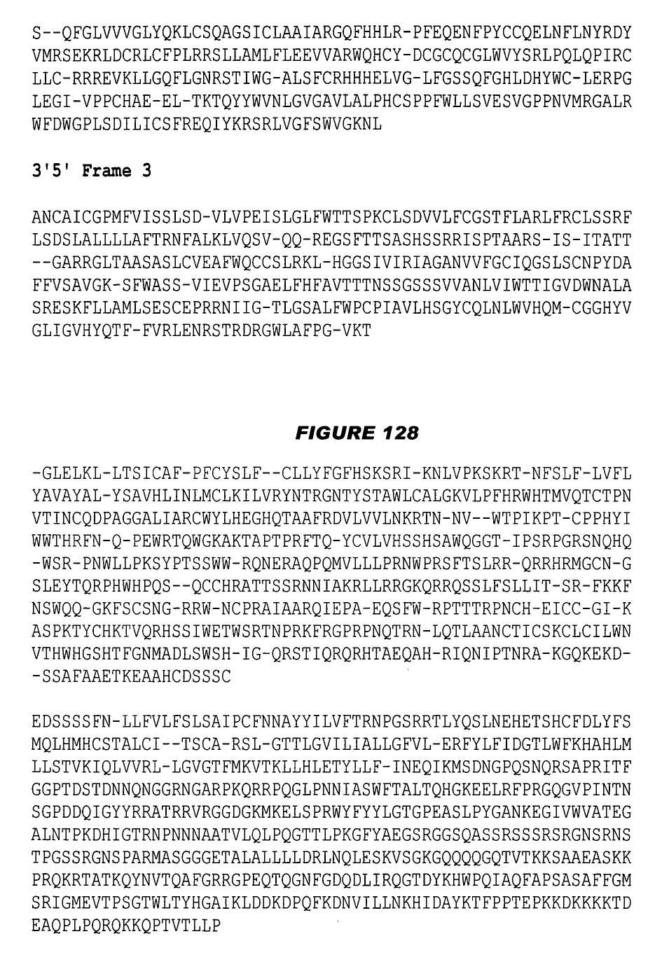

The invention includes a polynucleotide sequence SEQ ID NO: 11563 or a fragment thereof or a sequence having sequence identity thereto. Polypeptide sequences which can be translated from SEQ ID NO: 11563 are shown in FIG. 128. The constituent amino acid sequences from FIG. 128, having at least 4 amino acids, are listed as SEQ ID NO^{S}: 11564 to 11617.

The invention includes a polypeptide sequence selected from the group consisting of the sequences of FIG. 128, or a fragment thereof or a sequence having sequence identity thereto e.g. SEQ ID NO^{S}: 11563 to 11617.

A polypeptide sequence within SEQ ID NO: 11600 is SEQ ID NO: 11618. The invention includes a polypeptide comprising SEQ ID NO: 11618, or a fragment thereof or a sequence having sequence identity thereto.

A polypeptide sequence within SEQ ID NO: 11602 is SEQ ID NO: 11641. The invention includes a polypeptide comprising SEQ ID NO: 11641, or a fragment thereof or a sequence having sequence identity thereto.

A polypeptide sequence within SEQ ID NO: 11609 is SEQ ID NO: 11619.

The invention includes a polynucleotide encoding (i) an amino acid sequence selected from the group consisting of: (1) the amino acid sequences of FIG. 128, and in particular SEQ ID NO^{S}: 11564-11617; (2) SEQ ID NO: 11618; and (3) SEQ ID NO: 11619, or (ii) a fragment thereof. The invention includes a diagnostic kit comprising a one or more of these proteins. The invention includes a diagnostic kit comprising a polynucleotide sequence encoding one or more of these polypeptide sequences. The invention includes an antibody which recognizes one or more of the polypeptide sequences.

The SARS virus may contain polymorphism at isoleucine residue Ile-326 in SEQ ID NO: 11620 (Chi-PEP3). The invention includes a polypeptide comprising an amino acid sequence having sequence identity to SEQ ID NO: 11620, wherein said polypeptide includes an amino acid sequence selected from the group consisting of YAIHH (SEQ ID NO: 11621) and YATHH (SEQ ID NO: 11622). The invention includes a fragment of a polypeptide comprising SEQ ID NO: 11620, wherein said fragment includes an amino acid sequence selected from the group consisting of YAIHH (SEQ ID NO: 11621) and YATHH (SEQ ID NO: 11622).

The SARS virus may contain polymorphism at glutamine residue Gln-830 in SEQ ID NO: 11620. The invention includes a polypeptide comprising an amino acid sequence having sequence identity to SEQ ID NO: 11620, wherein said polypeptide includes an amino acid sequence selected from the group consisting of ASQAW (SEQ ID NO: 11623) and ASRAW (SEQ ID NO: 11624). The invention includes a fragment of a polypeptide comprising SEQ ID NO: 11620, wherein said fragment includes an amino acid sequence selected from the group consisting of ASQAW (SEQ ID NO: 11623) and ASRAW (SEQ ID NO: 11624).

The SARS virus may contain polymorphism at aspartic acid residue Asp-935 in SEQ ID NO: 11620. The invention includes a polypeptide comprising an amino acid sequence having sequence identity to SEQ ID NO: 11620, wherein said polypeptide includes an amino acid sequence selected from the group consisting of DADST (SEQ ID NO: 11625) and DAYST (SEQ ID NO: 11626). The invention includes a fragment of a polypeptide comprising SEQ ID NO: 11620, wherein said fragment includes an amino acid sequence selected from the group consisting of DADST (SEQ ID NO: 11625) and DAYST (SEQ ID NO: 11626).

The SARS virus may contain polymorphism at serine residue Ser-577 in SEQ ID NO: 11627 (Chi-PEP4). The invention includes a polypeptide comprising an amino acid sequence having sequence identity to SEQ ID NO: 11627, wherein said polypeptide includes an amino acid sequence selected from the group consisting of PCSFG (SEQ ID NO: 11628) and PCAFG (SEQ ID NO: 11629). The invention includes a fragment of a polypeptide comprising SEQ ID NO: 11627, wherein said fragment includes an amino acid sequence selected from the group consisting of PCSFG (SEQ ID NO: 11628) and PCAFG (SEQ ID NO: 11629).

The SARS virus may contain polymorphism at valine residue Val-68 in SEQ ID NO: 11630 (Chi-PEP8). The invention includes a polypeptide comprising an amino acid sequence having sequence identity to SEQ ID NO: 11630, wherein said polypeptide includes an amino acid sequence selected from the group consisting of LAVVY (SEQ ID NO: 11631) and LAAVY (SEQ ID NO: 11632). The invention includes a fragment of a polypeptide comprising SEQ ID NO: 11630, wherein said fragment includes an amino acid sequence selected from the group consisting of LAVVY (SEQ ID NO: 11631) and LAAVY (SEQ ID NO: 11632).

The SARS virus may contain polymorphism at isoleucine residue Ile-50 in SEQ ID NO: 11633 (Chi-PEP13). The invention includes a polypeptide comprising an amino acid sequence having sequence identity to SEQ ID NO: 11633, wherein said polypeptide includes an amino acid sequence selected from the group consisting of NNIAS (SEQ ID NO: 11634) and NNTAS (SEQ ID NO: 11635). The invention includes a fragment of a polypeptide comprising SEQ ID NO: 11633, wherein said fragment includes an amino acid sequence selected from the group consisting of NNIAS (SEQ ID NO: 11634) and NNTAS (SEQ ID NO: 11635).

The SARS virus may contain a polymorphism at Serine residue Ser-943 in SEQ ID NO: 11636. The invention includes a polypeptide comprising an amino acid sequence having sequence identity to SEQ ID NO: 11636, wherein said polypeptide includes an amino acid sequence selected from the group consisting of AVSAC (SEQ ID NO: 11637) and AVGAC (SEQ ID NO: 11638). The invention includes a fragment of a polypeptide comprising SEQ ID NO: 11636, wherein said fragment includes an amino acid seuence selected from the group consisting of AVSAC (SEQ ID NO: 11637) and AVGAC (SEQ ID NO: 11638).

The invention includes a polynucleotide SEQ ID NO: 11639, or a fragment thereof or a sequence having sequence identity thereto. The invention includes a polypeptide encoded by the polynucleotide sequence set forth in SEQ ID NO: 11639, or a fragment thereof or a polypeptide sequence having sequence identity thereto.

The invention includes a polynucleotide set forth in SEQ ID NO: 11640, or a fragment thereof or a sequence having sequence identity thereto. The invention includes a polypeptide encoded by the polynucleotide sequence set forth in SEQ ID NO: 11640, or a fragment thereof or a polypeptide sequence having sequence identity thereto.

The invention includes each of the polynucleotides identified above. The invention includes each of the polynucleotides set forth in the sequence listing. The invention further includes polynucleotides having sequence identity to each of the polynucleotides identified above. The degree of sequence identity is preferably greater than 50% (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more).

The invention includes polynucleotide sequences comprising fragments of each of the polynucleotide sequences identified above. The fragments should comprise at least n consecutive polynucleotides from a particular SEQ ID NO:, and, depending on the sequence, n is 7 or more (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 80, 90, 100, 120, 130, 140, 150, 160, 170, 180, 190, 200, 210, 220, 230, 240, 250, 260, 270, 280, 290, 300 or more).

The invention includes each of the amino acid sequences encoded by each of the polynucleotide sequences identified above. The invention includes each of the amino acid sequences encoded by each of the polynucleotide sequences set forth in the sequence listing. The invention further includes amino acid sequences having sequence identity to the amino acid sequences encoded by each of the polynucleotide sequences identified above. The degree of sequence identity is preferably greater than 50% (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more). The invention further includes fragments of amino acid sequences encoded by each of the polynucleotide sequences identified above. The fragments should comprise at least n consecutive amino acids from a particular SEQ ID NO:, and, depending on the sequence, n is 7 or more (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 80, 90, 100, 120, 130, 140, 150, 160, 170, 180, 190, 200, 210, 220, 230, 240, 250, 260, 270, 280, 290, 300 or more).

The invention includes each of the amino acid sequences identified above. The invention includes each of the amino acid sequence set forth in the sequence listing. The invention further includes amino acid sequences having sequence identity to each of the amino acid sequences identified above. The degree of sequence identity is preferably greater than 50% (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more).

The invention further includes fragments of the amino acid sequences identified above. The fragments should comprise at least n consecutive amino acids from a particular SEQ ID NO:, and, depending on the sequence, n is 7 or more (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 80, 90, 100, 120, 130, 140, 150, 160, 170, 180, 190, 200, 210, 220, 230, 240, 250, 260, 270, 280, 290, 300 or more).

The invention includes polynucleotides encoding each of the amino acid sequences identified above. The invention includes polynucleotides encoding each of the amino acid sequences set forth in the sequence listing. The invention further includes polynucleotides having sequence identity with each of the polynucleotides encoding each of the amino acid sequences identified above. The degree of sequence identity is preferably greater than 50% (e.g., 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more).

The invention further includes fragments of polynucleotides encoding each of the amino acid sequences identified above. The fragments should comprise at least n consecutive polynucleotides from a particular SEQ ID NO:, and, depending on the sequence, n is 7 or more (e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 80, 90, 100, 120, 130, 140, 150, 160, 170, 180, 190, 200, 210, 220, 230, 240, 250, 260, 270, 280, 290, 300 or more).

As described in more detail below, polynucleotides for use as primers and/or as probes may contain at least 4 or 8 contiguous nucleotides from a polynucleotide sequence of the invention e.g. at least 14, 15, 16, 17, 18, 19, 20, 21, 22, 23, 24, 25, 26, 27, 28, 29, or 30 contiguous nucleotides and up to about 50, 75, 100, 200 contiguous nucleotides or more. While 6-8 nucleotides may be a workable length, sequences of 10-12 nucleotides are preferred, and about 13, 14, 15, 16, 17, 18, 19, 20, or 21 or more nucleotides or more appears optimal for hybridisation.

In one embodiment, the invention is directed to polynucleotides and amino acid sequences that do not consist entirely of a known SARS virus polynucleotide or amino acid sequence or of a known coronavirus polynucleotide or amino acid sequence. In one embodiment, the polynucleotides and amino acid sequences of the invention do not consist entirely of the sequence SEQ ID NO: 1. In another embodiment, the polynucleotides and amino acid sequences of the invention do not consist entirely of the sequence SEQ ID NO: 2. SEQ ID NO: 9967 is a SARS genome sequence of the Frankfurt (FRA) isolate (GenBank: AY310120). Compared to SEQ ID NO: 1, it differs at nucleotides 2546, 2590, 11437, 18954, 19073, 20585, 20899, 23209, 24922, 26589 & 28257; compared to SEQ ID NO:2, it differs at nucleotides 2560, 7922, 11451, 16625, 18968 & 19067. Further genome sequences have become available from GenBank, since this application was originally filed, under accession numbers including AY559097, AY559096, AY559095, AY559094, AY559093, AY559092, AY559091, AY559090, AY559089, AY559088, AY559087, AY559086, AY559085, AY559084, AY559083, AY559082, AY559081, AY274119, AY323977, AY291315, AY502932, AY502931, AY502930, AY502929, AY502928, AY502927, AY502926, AY502925, AY502924, AY502923, AY291451, AY390556, AY395003, AY395002, AY395001, AY395000, AY394999, AY394998, AY394997, AY394996, AY394995, AY394994, AY394993, AY394992, AY394991, AY394990, AY394989, AY394987, AY394986, AY394985, AY394983, AY394979, AY394978, AY508724, AY394850, AY463059, AY463060, AY313906, AY310120, AY461660, AY485278, AY485277, AY345988, AY345987, AY345986, AY282752, AY357076, AY357075, AY350750, AY304495, AY304488, AY304486, AY427439, AY283798, AY278491, AY278489, AY362699, AY362698, AY283797, AY283796, AY283795, AY283794, AY278741, AY351680, AP006561, AP006560, AP006559, AP006558, AP006557, AY278554, AY348314, AY338175, AY338174, AY321118, AY279354, AY278490, AY278487, AY297028, AY278488, and ND_{—}004718.

In another embodiment, the invention is directed to polynucleotides that encode proteins which are not immunologically cross reactive with a protein of a mouse hepatitis virus, a bovine coronavirus or an avian infectious bronchitis virus. In another embodiment, the invention is directed to proteins which are not immunologically cross reactive with a protein of a mouse hepatitis virus, a bovine coronavirus or an avian infectious bronchitis virus.

Each of the polynucleotides identified above may be used to encode a portion of a fusion protein. Accordingly, the invention compries one or more of the polynucleotides identified above wherein the polynucleotides encoding for the start codon are removed. The invention further comprises one or more of the amino acids identified above wherein the starting methionine is removed.

Any of the polynucleotide or amino acid sequences discussed above may be used in vaccines for the treatment or prevention of SARS virus infection, including as a SARS viral antigen. Additionally, any of the polynucleotides or amino acid sequences discussed above may be used as diagnostic reagents, or in kits (comprising such reagents) or in methods used to diagnose or identify the presence or absence of a SARS virus in a biological sample.

SARS viral antigens of the invention may include a polypeptide with 99%, 95%, 90%, 85%, or 80% homology to one or more of the group consisting of the following proteins: nonstructural protein 2 (NS2); hemagglutinin-esterase glycoprotein (HE) (also referred to as E3), spike glycoprotein (S) (also referred to as E2), nonstructural region 4 (NS4), envelope (small membrane) protein (E) (also referred to as sM), membrane glycoprotein (M) (also referred to as E1), nucleocapsid phosphoprotein (N) or RNA dependent RNA polymerase (pol).

A detailed discussion of Coroavirus biology can be found in Fields Virology (2nd ed), Fields et al. (eds.), B.N. Raven Press, New York, N.Y., Chapter 35.

Another example of a SARS virus isolate is set forth in Example 1 below. The invention includes each of the polypeptide and polynucleotide sequences identified in Example 1. In addition, the invention includes vaccine formulations comprising one or more of the polypeptide or polynucleotide sequences identified in Example 1. The invention includes diagnostic regaents, kits (comprising such reagents) and methods which can be used to diagnose or identify the presence or absence of a SARS virus in a biological sample using one or more of the polypeptide or polynucleotide sequences identified in Example 1. The invention includes methods for the treatment or prevention of SARS virus infection utilizing small molecule viral inhibitors and combinations of small molecule viral inhibitors and kits for the treatment of SARS. The small molecule inhibitors may specifically target one or more of the polypeptides or polynucleotides identified in Example 1.

Further discussion of terms used in the application follows below.

“Respiratory Virus” as used herein refers to a virus capable of infecting the human respiratory tract. Respiratory Viral Antigens suitable for use in the invention include Severe Acute Respiratory Syndrome virus, coronavirus, influenza virus, human rhinovirus (HRV), parainfluenza virus (PWV), respiratory syncytial virus (RSV), adenovirus, metapneumovirus, and rhinovirus.

The terms “polypeptide”, “protein” and “amino acid sequence” as used herein generally refer to a polymer of amino acid residues and are not limited to a minimum length of the product. Thus, peptides, oligopeptides, dimers, mulimers, and the like, are included within the definition. Both full-length proteins and fragments thereof are encompassed by the definition. Minimum fragments of polypeptides useful in the invention can be at least 3, 4, 5, 6, 7, 8, 9, 10, 11, 12, 13, 14, or even 15 amino acids. Typically, polypeptides useful in this invention can have a maximum length suitable for the intended application. Generally, the maximum length is not critical and can easily be selected by one skilled in the art.

Polypeptides of the invention can be prepared in many ways e.g. by chemical synthesis (at least in part), by digesting longer polypeptides using proteases, by translation from RNA, by purification from cell culture (e.g. from recombinant expression), from the organism itself (e.g. after viral culture, or direct from patients), from a cell line source etc. A preferred method for production of peptides <40 amino acids long involves in vitro chemical synthesis (Bodanszky (1993) Principles of Peptide Synthesis (ISBN: 0387564314); Fields et al. (1997) Methods in Enzymology 289: Solid-Phase Peptide Synthesis. ISBN: 0121821900). Solid-phase peptide synthesis is particularly preferred, such as methods based on t-Boc or Fmoc (Chan & White (2000) Fmoc Solid Phase Peptide Synthesis ISBN: 0199637245) chemistry. Enzymatic synthesis (Kullmann (1987) Enzymatic Peptide Synthesis. ISBN: 0849368413) may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) (Ibba (1996) Biotechnol Genet Eng Rev 13:197-216.). Where D-amino acids are included, however, it is preferred to use chemical synthesis. Polypeptides of the invention may have covalent modifications at the C-terminus and/or N-terminus, particularly where they are for in vivo administration e.g by attachment of acetyl or carboxamide, as in the Fuzeon™ product.

Reference to polypeptides and the like also includes derivatives of the amino acid sequences of the invention. Such derivatives can include postexpression modifications of the polypeptide, for example, glycosylation, acetylation, phosphorylation, and the like. Amino acid derivatives can also include modifications to the native sequence, such as deletions, additions and substitutions (generally conservative in nature), so long as the protein maintains the desired activity. These modifications may be deliberate, as through site-directed mutagenesis, or may be accidental, such as through mutations of.hosts which produce the proteins or errors due to PCR amplification. Furthermore, modifications may be made that have one or more of the following effects: reducing toxicity; facilitating cell processing (e.g., secretion, antigen presentation, etc.); and facilitating presentation to B-cells and/or T-cells.

“Fragment” or “Portion” as used herein refers to a polypeptide consisting of only a part of the intact full-length polypeptide sequence and structure as found in nature. For instance, a fragment can include a C-terminal deletion and/or an N-terminal deletion of a protein.

A “recombinant” protein is a protein which has been prepared by recombinant DNA techniques as described herein. In general, the gene of interest is cloned and then expressed in transformed organisms, as described further below. The host organism expressed the foreign gene to produce the protein under expression conditions.

The term “polynucleotide”, as known in the art, generally refers to a nucleic acid molecule. A “polynucleotide” can include both double- and single-stranded sequences and refers to, but is not limited to, cDNA from viral, prokaryotic or eukaryotic mRNA, genomic RNA and DNA sequences from viral (e.g. RNA and DNA viruses and retroviruses) or prokaryotic DNA, and especially synthetic DNA sequences. The term also captures sequences that include any of the known base analogs of DNA and RNA, and includes modifications such as deletions, additions and substitutions (generally conservative in nature), to the native sequence, so long as the nucleic acid molecule encodes a therapeutic or antigenic protein. These modifications may be deliberate, as through site-directed mutagenesis, or may be accidental, such as through mutations of hosts that produce the antigens. Modifications of polynucleotides may have any number of effects including, for example, facilitating expression of the polypeptide product in a host cell.

Polynucleotides of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

A polynucleotide can encode a biologically active (e.g., immunogenic or therapeutic) protein or polypeptide. Depending on the nature of the polypeptide encoded by the polynucleotide, a polynucleotide can include as little as 10 nucleotides, e.g., where the polynucleotide encodes an antigen.

By “isolated” is meant, when referring to a polynucleotide or a polypeptide, that the indicated molecule is separate and discrete from the whole organism with which the molecule is found in nature or, when the polynucleotide or polypeptide is not found in nature, is sufficiently free of other biological macromolecules so that the polynucleotide or polypeptide can be used for its intended purpose. The polynucleotides and polypeptides of the invention are preferably isolated polynucleotides and isolated polypeptides.

“Antibody” as known in the art includes one or more biological moieties that, through chemical or physical means, can bind to or associate with an epitope of a polypeptide of interest. The antibodies of the invention include antibodies which specifically bind to a SARS viral antigen. The term “antibody” includes antibodies obtained from both polyclonal and monoclonal preparations, as well as the following: hybrid (chimeric) antibody molecules (see, for example, Winter et al. (1991) Nature 349: 293-299; and U.S. Pat. No. 4,816,567; F(ab′)₂ and F(ab) fragments; Fᵥ molecules (non-covalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5897-5883); dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, for example, Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain immunological binding properties of the parent antibody molecule. The term “antibody” further includes antibodies obtained through non-conventional processes, such as phage display.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human rather than murine hybridomas. See, e.g., Cote, et al. Monoclonal Antibodies and Cancer Therapy, Alan R. Liss, 1985, p 77.

An “immunogenic composition” as used herein refers to a composition that comprises an antigenic molecule where administration of the composition to a subject results in the development in the subject of a humoral and/or a cellular immune response to the antigenic molecule of interest. The immunogenic composition can be introduced directly into a recipient subject, such as by injection, inhalation, oral, intranasal or any other parenteral, mucosal or transdermal (e.g., intra-rectally or intra-vaginally) route of administration.

The term “derived from” is used to identify the source of molecule (e.g., a molecule can be derived from a polynucleotide, polypeptide, an immortalized cell line can be derived from any tissue, etc.). A first polynucleotide is “derived from” a second polynucleotide if it has the same or substantially the same basepair sequence as a region of the second polynucleotide, its cDNA, complements thereof, or if it displays sequence identity as described above. Thus, a first polynucleotide sequence is “derived from” a second sequence if it has (i) the same or substantially the same sequence as the second sequence or (ii) displays sequence identity to polypeptides of that sequence.

A first polypeptide is “derived from” a second polypeptide if it is (i) encoded by a first polynucleotide derived from a second polynucleotide, or (ii) displays sequence identity to the second polypeptides as described above. Thus, a polypeptide (protein) is “derived from” a particular SARS virus if it is (i) encoded by an open reading frame of a polynucleotide of that SARS virus, or (ii) displays sequence identity, as described above, to polypeptides of that SARS virus.

Both polynucleotide and polypeptide molecules can be physically derived from a SARS virus or produced recombinantly or synthetically, for example, based on known sequences.

A cultured cell or cell line is “derived from” another cell, cells or tissue if it is originally obtained from existing cells or tissue. Non-limiting examples of tissue that cells may be derived from include skin, retina, liver, kidney, heart, brain, muscle, intestinal, ovary, breast, prostate, cancerous tissue, tissue infected with one or more pathogens (e.g., viruses, bacteria etc.) and the like. The cells described herein may also be derived from other cells including, but not limited to, primary cultures, existing immortalized cells line and/or other isolated cells.

An “antigen” refers to a molecule containing one or more epitopes (either linear, conformational or both) that will stimulate a host's immune system to make a humoral and/or cellular antigen-specific response. The term is used interchangeably with the term “immunogen.” Normally, an epitope will include between about 3-15, generally about 5-15 amino acids. A B-cell epitope is normally about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term “antigen” denotes both subunit antigens, (i.e., antigens which are separate and discrete from a whole organism with which the antigen is associated in nature), as well as, killed, attenuated or inactivated bacteria, viruses, fungi, parasites or other microbes as well as tumor antigens, including extracellular domains of cell surface receptors and intracellular portions that may contain T-cell epitopes. Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide that expresses an antigen or antigenic determinant in vivo, such as in gene therapy and DNA immunization applications, is also included in the definition of antigen herein.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, including secretory (IgA) or IgG molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. In addition, a chemokine response may be induced by various white blood or endothelial cells in response to an administered antigen.

II. Vaccine Formulations

The invention relates to vaccine formulations for the treatment or prevention of Severe Acute Respiratory Syndrome (SARS). Vaccine formulations of the invention include an inactivated (or killed) SARS virus, an attenuated SARS virus, a split SARS virus preparation and a recombinant or purified subunit formulation of one or more SARS viral antigens. The invention includes polypeptides and polynucleotides encoding for SARS viral antigens and immunogenic fragments thereof. Expression and delivery of the polynucleotides of the invention may be facilitated via viral vectors and/or viral particles, including Virus Like Particles (VLPs).

A. Inactivated (or Killed) SARS Vaccines

The invention includes a composition comprising an inactivated (or killed) SARS virus and methods for the production thereof. Inactivated SARS viral compositions can be used as prophylactic or therapeutic SARS virus vaccine. Preferably the inactivated SARS virus vaccine composition comprises an amount of inactivated SARS virus which, before inactivation, is equivalent to a virus titer of from about 4 to 7 logs plaque forming units (PFU) or 4 to 7 logs tissue culture infectious dose 50 (TCID₅₀) per milliliter. More preferably, before inactivation the virus titer is from 4 to 11, 7 to 11 or 9 to 11 PFU or TCID₅₀. Still more preferably the inactivated SARS virus vaccine composition comprises an amount of inactivated SARS virus which, before inactivation, is equivalent to a virus titer of from about 5 to 9 PFU or 5 to 9 TCID₅₀ per milliliter. In one embodiment, the PFU or TCID₅₀ of the cultured SARS virus at harvest is 6 to 8, more preferably about 7.5 PFU or TCID₅₀ per milliliter. Upon concentration of the viral harvest, the PFU or TCID₅₀ is preferably 8 to 11, still more preferably about 9 PFU or TCID₅₀ per milliliter. The vaccine composition comprises a sufficient amount of the SARS virus antigen to produce an immunological response in a primate.

Methods of inactivating or killing viruses are known in the art to destroy the ability of the viruses to infect mammalian cells. Such methods include both chemical or physical means. Chemical means for inactivating a SARS virus include treatment of the virus with an effective amount of one or more of the following agents: detergents, formaldehyde, formalin, β-propiolactone, or UV light. Additional chemical means for inactivation include treatment with methylene blue, psoralen, carboxyfullerene (C60) or a combination of any thereof. Other methods of viral inactivation are known in the art, such as for example binary ethylamine, acetyl ethyleneimine, or gamma irradiation.

For example formaldehyde may be used at concentrations such as 0.1 to 0.02%, preferably at 0.02 to 0.1%, and still more preferably at 0.04 to 0.05%. The inactivating agent is added to virus containing culture supernatants, prior to or after harvesting said culture supernatants from vessels used for virus propagation, either with or without a step of cell disruption for release of cell-associated virus prior to harvesting. Further, the inactivating agent may be added after said culture supernatants have been stored frozen and thawed, or after one or more steps of purification to remove cell contaminants. Preferably, however, formaldehyde is added after removal of cells and cellular debris or after one or more purification steps. After addition of formaldehyde, the virus containing mixture is transferred into an incubation vessel and incubated at refrigeration temperatures (e.g. +2 to 8° C.) or alternatively at elevated temperatures, such as ambient temperatures between approximately 20 and 30° C. or at 33° C. to 37° C. for a period of 12 hours to 7 days, whereby the temperature chosen should be adjusted to the duration of incubation. Prefered conditions are e.g. +2-8° C. for 3-7 days (prefered are 3-7days), ambient temperatures and incubation for 16 hours to 3 days (prefered 24-48 hours), or 35-37° C. for 12-36 hours. If it is desirable to remove excess formalin, sodium thiosulfate or sodium metabisulfite at equimolar or 1.5-fold molar concentration (relative to formaldehyde) may be added after completing the inactivation process.

For example, β-propiolactone may be used at concentrations such as 0.01 to 0.5%, preferably at 0.5% to 0.2%, and still more preferably at 0.025 to 0.1%. The inactivating agent is added to virus containing culture supernatants (virus material) prior to or after harvesting said culture supernatants from vessels used for virus propagation, either with or without a step of cell disruption for release of cell-associated virus prior to harvesting. Further, the inactivating agent may be added after said culture supernatants have been stored frozen and thawed, or after one or more steps of purification to remove cell contaminants. β-propiolactone is added to the virus material, with the adverse shift in pH to acidity being controlled with sodium hydroxide (e.g., 1 N NaOH), a Tris-buffer or sodium bicarbonate solution. After transfering the mixture to another inactivation vessel, the combined inactivating agent-virus materials are incubated at temperatures from 4° C. to 37° C., for incubation times of preferably 24 to 72 hours.

Another inactivant which may be used is binary ethyleneimine (BEI). Equal volumes of a 0.2 molar bromoethylamine hydrobromide solution and a 0.4 molar sodium hydroxide solution are mixed and incubated at about 37° C. for 60 minutes. The resulting cyclized inactivant is binary ethyleneimine, which is added to the virus materials at 0.5 to 4 percent, and preferably at 1 to 3 percent, volume to volume. The inactivating virus materials are held from about 4° C. to 37° C. for 24 to 72 hours with periodic agitation. At the end of this incubation 20 ml. of a sterile 1 molar sodium thiosulfate solution was added to insure neutralization of the BEI.

In one embodiment, the invention includes an inactivating method is designed to maximize exposure of the virus to the inactivating agent and to minimize long-term exposure of the temperature sensitive SARS virus particles to elevated temperatures. The invention includes an inactivation method comprising exposing the virus to the inactivation agent (such as BPL) for 12 to 24 hours at refrigeration temperatures followed by hydrolysis of any residual inactivating agent by elevating the temperature for only 3 hours. Preferably, the refrigeration temperatures are between 0 and 8° C., more preferably around 4° C. Preferably, the elevated temperature is between 33 and 41° C., more preferably around 37° C. As assessed by a test for residual infectious virus using 10 ml aliquots of the inactivated preparation, the method is able to inactivate SARS-CoV in raw cell culture harvests below a theoretical limit of 0.03 infectious units/ml.

Diluted and undiluted samples of the inactivated virus materials are added to susceptible cell (tissue) culture (e.g., VERO) to detect any non-inactivated virus. The cultured cells are passaged multiple times and examined for the presence of SARS virus based on any of a variety of methods, such as, for example, cytopathic effect (CPE) and antigen detection (e.g., via fluoroscent antibody conjugates specific for SARS virus). Such tests allow determination of complete virus inactivation.

Prior to inactivation, the SARS virus will be cultured in a mammalian cell culture. The cell culture may be adherently growing cells or cells growing in suspension. Preferably the cells are of mammalian origin, but may also be derived from avian (e.g., hens' cells such as hens' embryo cells (CEF cells)), amphibian, reptile, insect, or fish sources. Mammalian sources of cells include, but are not limited to, human or non-human primate (e.g., MRC-5 (ATCC CCL-171), WI-38 (ATCC CCL-75), HeLa cells, human diploid cells, fetal rhesus lung cells (e.g. ATCC CL-160), human embryonic kidney cells (293 cells, typically transformed by sheared adenovirus type 5 DNA), VERO cells (e.g., from monkey kidneys), horse, cow (e.g., MDBK cells), sheep, dog (e.g., MDCK cells from dog kidneys, ATCC CCL34 MDCK (NBL2) or MDCK 33016, deposit number DSM ACC 2219 as described in WO 97/37001), cat, and rodent (e.g., hamster cells such as BHK21-F, HKCC cells, or Chinese hamster ovary cells (CHO cells)), and may be obtained from a wide variety of developmental stages, including for example, adult, neonatal, fetal, and embryo.

In certain embodiments the cells are immortalized (e.g., PERC.6 cells are described, for example, in WO 01/38362 and WO 02/40665, incorporated by reference herein in their entireties, as well as deposited under ECACC deposit number 96022940), or any other cell type immortalized using the techniques described herein.

In preferred embodiments, mammalian cells are utilized, and may be selected from and/or derived from one or more of the following non-limiting cell types: fibroblast cells (e.g., dermal, lung), endothelial cells (e.g., aortic, coronary, pulmonary, vascular, dermal microvascular, umbilical), hepatocytes, keratinocytes, immune cells (e.g., T cell, B cell, macrophage, NK, dendritic), mammary cells (e.g., epithelial), smooth muscle cells (e.g., vascular, aortic, coronary, arterial, uterine, bronchial, cervical, retinal pericytes), melanocytes, neural cells (e.g., astrocytes), prostate cells (e.g., epithelial, smooth muscle), renal cells (e.g., epithelial, mesangial, proximal tubule), skeletal cells (e.g., chondrocyte, osteoclast, osteoblast), muscle cells (e.g., myoblast, skeletal, smooth, bronchial), liver cells, retinoblasts, and stromal cells. WO 97/37000 and WO 97/37001, incorporated by reference herein in their entireties, describe production of animal cells and cell lines that capable of growth in suspension and in serum free media and are useful in the production and replication of viruses.

Preferably, the SARS viruses of the invention are grown on VERO cells or fetal rhesus kidney cells.

Culture conditions for the above cell types are well-described in a variety of publications, or alternatively culture medium, supplements, and conditions may be purchased commercially, such as for example, as described in the catalog and additional literature of Cambrex Bioproducts (East Rutherford, N.J.).

In certain embodiments, the host cells used in the methods described herein are cultured in serum free and/or protein free media. A medium is referred to as a serum-free medium in the context of the present invention in which there are no additives from serum of human or animal origin. Protein-free is understood to mean cultures in which multiplication of the cells occurs with exclusion of proteins, growth factors, other protein additives and non-serum proteins. The cells growing in such cultures naturally contain proteins themselves.

Known serum-free media include Iscove's medium, Ultra-CHO medium (BioWhittaker) or EX-CELL (JRH Bioscience). Ordinary serum-containing media include Eagle's Basal Medium (BME) or Minimum Essential Medium (MEM) (Eagle, Science, 130, 432 (1959)) or Dulbecco's Modified Eagle Medium (DMEM or EDM), which are ordinarily used with up to 10% fetal calf serum or similar additives. Optionally, Minimum Essential Medium (MEM) (Eagle, Science, 130, 432 (1959)) or Dulbecco's Modified Eagle Medium (DMEM or EDM) may be used without any serum containing supplement. Protein-free media like PF-CHO (JHR Bioscience), chemically-defined media like ProCHO 4CDM (BioWhittaker) or SMIF 7 (Gibco/BRL Life Technologies) and mitogenic peptides like Primactone, Pepticase or HyPep™ (all from Quest International) or lactalbumin hydrolyzate (Gibco and other manufacturers) are also adequately known in the prior art. The media additives based on plant hydrolyzates have the special advantage that contamination with viruses, mycoplasma or unknown infectious agents can be ruled out.

The cell culture conditions to be used for the desired application (temperature, cell density, pH value, etc.) are variable over a very wide range owing to the suitability of the cell line employed according to the invention and can be adapted to the requirements of the SARS virus.

The method for propagating the SARS virus in cultured cells (e.g., mammalian cells) includes the steps of inoculating the cultured cells with SARS virus, cultivating the infected cells for a desired time period for virus propagation, such as for example as determined by SARS virus titer or SARS virus antigen expression (e.g., between 24 and 168 hours after inoculation) and collecting the propagated virus. The cultured cells are inoculated with a SARS virus (measured by PFU or TCID₅₀) to cell ratio of 1:10000 to 1:10. A lower range of ratios may also be used e.g. 1:500 to 1:1, preferably 1:100 to 1:5, more preferably 1:50 to 1:10. The SARS virus is added to a suspension of the cells or is applied to a monolayer of the cells, and the virus is absorbed on the cells for at least 60 minutes but usually less than 300 minutes, preferably between 90 and 240 minutes at 25° C to 40° C., more preferably 28° C. to 37° C., still more preferably at about 33° C. The infected cell culture (e.g., monolayers) may be treated either by freeze-thawing or by enzymatic action to increase the viral content of the harvested culture supernatants. The harvested fluids are then either inactivated or stored frozen.

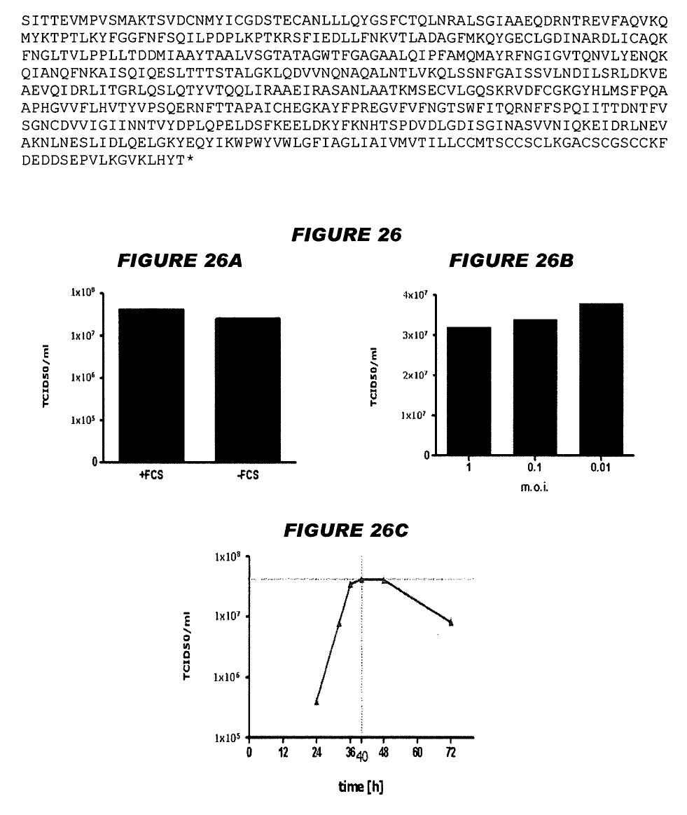

A comparison of SARS infected Vero cells grown with and without fetal calf serum (“FCS”) is shown in FIG. 26A. Briefly, Vero cells were split the day before infection and cultivated in T175 flasks. Infection of 90% confluent Vero cell monolayers the following day was performed with a SARS-CoV seed stock (strain FRA, passage 4, Accession number AY310120), with or without 3% FCS (FIG. 26A). The addition of FCS to the cell media showed little impact on virus yield.

Cultured cells may be infected at a multiplicity of infection (“m.o.i.”) of about 0.0001 to 10, preferably 0.002 to 5, more preferably to 0.001 to 2. Still more preferably, the cells are infected at a m.o.i of about 0.01. A comparison of viral yield at varying m.o.i. levels is shown in FIG. 26B.

Infected cells may be harvested 30 to 60 hours post infection. Preferably, the cells are harvested 34-48 hours post infection. Still more preferably, the cells are harvested 38 to 40 hours post infection. See FIG. 26C.

Methods of purification of inactivated virus are known in the art and may include one or more of, for instance gradient centrifugation, ultracentrifugation, continuous-flow ultracentrifugation and chromatography, such as ion exchange chromatography, size exclusion chromatography, and liquid affinity chromatography. Additional method of purification include ultrafiltration and dialfiltration. See J P Gregersen “Herstellung von Virussimpfstoffen aus Zellkulturen” Chapter 4.2 in Pharmazeutische Biotecnology (eds. O. Kayser and R H Mueller) Wissenschaftliche Verlagsgesellschaft, Stuttgart, 2000. See also, O'Neil et al., “Virus Harvesting and Affinity Based Liquid Chromatography. A Method for Virus Concentration and Purification”, Biotechnology (1993) 11:173-177; Prior et al., “Process Development for Manufacture of Inactivated HIV-1”, Pharmaceutical Technology (1995) 30-52; and Majhdi et al., “Isolation and Characterization of a Coronavirus from Elk Calves with diarrhea” Journal of Clinical Microbiology (1995) 35(11): 2937-2942.

Other examples of purification methods suitable for use in the invention include polyethylene glycol or ammonium sulfate precipitation (see Trepanier et al., “Concentration of human respiratory syncytial virus using ammonium sulfate, polyethylene glycol or hollow fiber ultrafiltration” Journal of Virological Methods (1981) 3(4):201-211; Hagen et al., “Optimization of Poly(ethylene glycol) Precipitation of Hepatitis Virus Used to prepare VAQTA, a Highly Purified Inactivated Vaccine” Biotechnology Progress (1996) 12:406-412; and Carlsson et al., “Purification of Infectious Pancreatic Necrosis Virus by Anion Exchange Chromatography Increases the Specific Infectivity” Journal of Virological Methods (1994) 47:27-36) as well as ultrafiltration and microfiltration (see Pay et al., Developments in Biological Standardization (1985) 60:171-174; Tsurumi et al., “Structure and filtration performances of improved cuprammonium regenerated cellulose hollow fibre (improved BMM hollow fibre) for virus removal” Polymer Journal (1990) 22(12):1085-1100; and Makino et al., “Concentration of live retrovirus with a regenerated cellulose hollow fibre, BMM”, Archives of Virology (1994) 139(1-2):87-96.).

Preferably, the virus is purified using chromatography, such as ion exchange, chromatography. Chromatic purification allows for the production of large volumes of virus containing suspension. The viral product of interest can interact with the chromatic medium by a simple adsorption/desorption mechanism, and large volumes of sample can be processed in a single load. Contaminants which do not have affinity for the adsorbent pass through the column. The virus material can then be eluted in concentrated form.

Preferred anion exchange resins for use in the invention include DEAE, EMD TMAE. Preferred cation exchange resins may comprise a sulfonic acid-modified surface. In one embodiment, the virus is purified using ion exchange chromatography comprising a strong anion exchange resin (e.g. EMD TMAE) for the first step and EMD-SO₃ (cation exchange resin) for the second step. A metal-binding affinity chromatography step can optionally be included for further purification. (See, e.g., WO 97/06243).

A preferred resin for use in the invention is Fractogel™ EMD. This synthetic methacrylate based resin has long, linear polymer chains (so-called “tentacles”) covalently attached. This “tentacle chemistry” allows for a large amount of sterically accessible ligands for the binding of biomolecules without any steric hindrance. This resin also has improved pressure stability.

Column-based liquid affinity chromatography is another preferred purification method for use in the invention. One example of a resin for use in this purification method is Matrex™ Cellufine™ Sulfate (MCS). MCS consists of a rigid spherical (approx. 45-105 μm diameter) cellulose matrix of 3,000 Dalton exclusion limit (its pore structure excludes macromolecules), with a low concentration of sulfate ester functionality on the 6-position of cellulose. As the functional ligand (sulfate ester) is relatively highly dispersed, it presents insufficient cationic charge density to allow for most soluble proteins to adsorb onto the bead surface. Therefore the bulk of the protein found in typical virus pools (cell culture supernatants, e.g. pyrogens and most contaminating proteins, as well as nucleic acids and endotoxins) are washed from the column and a degree of purification of the bound virus is achieved.

The rigid, high-strength beads of MCS tend to resist compression. The pressure/flow characteristics the MCS resin permit high linear flow rates allowing high-speed processing, even in large columns, making it an easily scalable unit operation. In addition a chromatographic purification step with MCS provides increased assurance of safety and product sterility, avoiding excessive product handling and safety concerns. As endotoxins do not bind to it, the MCS purification step allows a rapid and contaminant free depyrogenation. Gentle binding and elution conditions provide high capacity and product yield. The MCS resin therefore represents a simple, rapid, effective, and cost-saving means for concentration, purification and depyrogenation. In addition, MCS resins can be reused repeatedly.

The inactivated virus may be further purified by gradient centrifugation, preferably density gradient centrifugation. For commercial scale operation a continuous flow sucrose gradient centrifugation would be the preferred option. This method is widely used to purify antiviral vaccines and is known to the expert in the field (See J P Gregersen “Herstellung von Virussimpfstoffen aus Zellkulturen” Chapter 4.2 in Pharmazeutische Biotechnology (eds. O. Kayser and R H Mueller) Wissenschaftliche Verlagsgesellschaft, Stuttgart, 2000.)

The density gradient centrifugation step may be performed using laboratory or commercial scale gradient centrifugation equipment. For example, a swinging bucket rotor, a fixed angle rotor, or a vertical tube rotor, particularly for laboratory scale production of the virus. Preferably, the gradient centrifugation step is performed using a swinging bucket rotor. This type of rotor has a sufficiently long pathlength to provide high quality separations, particularly with multicomponent samples. In addition, swinging bucket rotors have greatly reduced wall effects, and the contents do not reorient during acceleration and deceleration. Because of their longer pathlength, separations take longer compared to fixed angle or vertical tube rotors. The prepared sucrose solutions are controlled via refractometer on their sucrose concentration.

Sucrose gradients for density gradient centrifugation, such as in a swinging bucket centrifuge tubes may be formed prior to centrifugation by the use of a gradient former (continuous/linear). The volume of sample which can be applied to the gradient in a swinging bucket rotor tube is a function of the cross-sectional area of the gradient that is exposed to the sample. If the sample volume is too high, there is not sufficient radial distance in the centrifuge tube for effective separation of components in a multicomponent sample.

An approximate sample volume for swinging bucket rotor SW 28 is 1-5 ml per tube (with a tube diameter of 2.54 cm). The sample is applied to the gradient by pipetting the volume on top of the gradient. The blunt end of the pipette is placed at 45-60° angle to the tube wall, approximately 2-3 mm above the gradient. The sample is injected slowly and allowed to run down the wall of the tube onto the gradient. After centrifugation gradient fractions are recovered by carefully inserting a gauge needle until the bottom of the tube and starting to collect fractions of 2 ml by pumping the liquid from the tube into falcon tubes.

Sucrose density gradients suitable for use with this density gradient centrifugation purification step include 0-60%, 5-60%, 15-60%, 0-50%, 5-50%, 15-50%, 0-40%, 5-40%, and 15-40%. Preferably, the sucrose density gradient is 15-40%, 5-40% or 0-40%.

Alternatively, a discontinuous sucrose density gradient may be used for purification. A discontinuous sucrose density scheme provides for discrete, overlaying layers of differing sucrose concentrations. In one example, a first layer of 50% sucrose is covered by a second layer of 40% sucrose; the second layer is covered by a third layer of 20% sucrose; the third layer is covered by a fourth layer of 10% sucrose; and the fourth layer is covered by the solution containing the virus to be purified.

In one embodiment, inactivated virus is purified by a method comprising a first step of chromatography purification and a second step of gradient centrifugation. Preferably the first step comprises liquid affinity chromatography, such as MCS. Preferably, the second step comprises density gradient centrifugation using a swinging bucket rotor.

Additional purification methods which may be used to purify inactivated SARS virus include the use of a nucleic acid degrading agent, preferably a nucleic acid degrading enzyme, such as a nuclease having DNase and RNase activity, or an endonuclease, such as from Serratia marcescens, commercially available as Benzonase™, membrane adsorbers with anionic functional groups (e.g. Sartobind™) or additional chromatographic steps with anionic functional groups (e.g. DEAE or TMAE). An ultrafiltration/dialfiltration and final sterile filtration step could also be added to the purification method.

Preferably, the purification includes treatment of the SARS viral isolate with one or more nucleic acid degrading enzymes. These enzymes may be used to reduce the level of host cell nucleic acid in the viral purification process. Nucleic acid digesting enzymes for use in cell culture are known in the art and include, for example, Benzonase™.

The treatment of the virus with the nucleic acid degrading enzyme and inactivating agent can be performed by a sequential treatment or in a combined or simultaneous manner. Preferably, the nucleic acid degrading agent is added to the virus preparation prior to the addition of the inactivating agent.

The purified viral preparation of the invention is substantially free of contaminating proteins derived from the cells or cell culture and preferably comprises less than about 1000, 500, 250, 150, 100, or 50 pg cellular nucleic acid/μg virus antigen, preferably less than about 1000, 500, 250, 150, 100, or 50 pg cellular nucleic acid/dose. Still more preferably, the purified viral preparation comprises less than about 20 pg, and even more preferably, less than about 10 pg. Methods of measuring host cell nucleic acid levels in a viral sample are known in the art. Standardized methods approved or recommended by regulatory authorities such as the WHO or the FDA are preferred.

The invention includes an inactivated vaccine composition comprising a prophylactically effective amount of SARS viral antigen, preferably spike or an immunogenic fragment thereof. The SARS viral antigen is preferably present in a concentration amount of 0.1 to 50 μg antigen/dose, more preferably 0.3 to 30 μg antigen/dose. Still more preferably, the antigen is about 15 μg/dose.

In one embodiment, a lower concentration of SARS viral antigen is used in inactivated vaccine compositions of the invention. Such lower concentration vaccines may optionally comprise an adjuvant to boost the host immune response to the antigen. In such a “low dose” vaccine, the SARS viral antigen is preferably present in a concentration of less than 15 μg antigen/dose, (i.e., less than 10, 7.5, 5 or 3 μg antigen/dose.

The inactivated vaccine preparations of the invention may further comprise a stabilizer to preserve the integrity of the immunogenic proteins in the inactivated viral preparation. Stabilizers suitable for use in vaccines are known in the art and may include, for example, buffers, sugars, sugar alcohols, and amino acids. Stabilizing buffers are preferably adjusted to a physiological pH range and may include phosphate buffers, Tris buffers, TE (Tris/EDTA), TEN (Tris/NaCl/EDTA) and Earle's salt solution. Stabilizing sugars may include, for example, one or more of saccharose, glucose, fructose, dextranes, dextranesulphate, and trehalose. Stabilizing sugar alcohols may include, for example, Xylite/Xylitole, Mannite/Mannitol, Sorbite/Sorbitol, and Glycerol. Amino acids suitable for use in the invention include, for example, L-glutamine, arginine, cysteine, and lysine. Additional stabilizers which may be used in the invention include Tartaric acid, Pluronic F 68, and Tween 80.

SARS viral isolates which may be used for the inactivated viral preparations of the invention may be obtained and identified by any of the mechanisms described supra. For example, a SARS isolate may be obtained from a clinical sample and plaque purified. Such methods of viral isolation are known in the art.

Further purification procedures can be applied to ensure the seed virus used for preparation of the vaccine does not contain, for example, unwanted adventitious agents. In one embodiment, viral RNA from the viral isolate can be isolated from the virus, purified (and, optionally, the sequence verified through PCR or other means) and then introduced into a suitable cell culture.

As an example of this technique, a clinical viral sample is plaque purified and amplified on vero cells to generate a sufficient amount of the viral sample for analysis. Cellular remnants are then cleared from the supernatant by centrifugation. The virus can then be pelleted by ultracentrifugation and the pellet resuspended in PBS. After further centrifugation purification, the virus containing fraction is treated with a DNase (and optionally also an RNase). Viral RNA is then isolated from this fraction and transfected into a host cell.

Examples 2 and 3 provide an illustration of purification of inactivated whole SARS virus using MCS chromatography resin purification followed by density gradient ultracentrifugation.

Routes and methods of immunization of the vaccines of the invention are discussed in more detail in a section below. Examples 4 and 5 provide illustrations of a mouse immunization scheme with the inactivated SARS virus of the invention.

B. Attenuated SARS Vaccines

The invention includes a composition comprising an attenuated SARS virus. This composition can be used as a prophylactic or therapeutic SARS virus vaccine. Methods of attenuating viruses are known in the art. Such methods include serial passage of the SARS virus in cultured cells (e.g., mammalian cell culture, preferably fetal rhesus kidney cells or VERO cells—see the discussion in Section A above regarding culture of SARS virus), until the SARS virus demonstrates attenuated function. The temperature at which the virus is grown can be any temperature at which with tissue culture passage attenuation occurs. Attenuated function of the SARS virus after one or more passages in cell culture can be measured by one skilled in the art. As used herein, attenuation refers to the decreased virulence of the SARS virus in a human subject. Evidence of attenuated function may be indicated by decreased levels of viral replication or by decreased virulence in an animal model.

Other methods of producing an attenuated SARS virus include passage of the virus in cell culture at sub-optimal or “cold” temperatures and introduction of attenuating mutations into the SARS viral genome by random mutagenesis (e.g., chemical mutagenesis) or site specific directed mutagenesis. Preparation and generation of attenuated RSV vaccines (the methods of which will generally applicable to SARS virus) are disclosed in, for example, EP 0 640 128, U.S. Pat. No. 6,284,254, U.S. Pat. No. 5,922,326, U.S. Pat. No. 5,882,651.

The attenuated derivatives of SARS virus are produced in several ways, such as for example, by introduction of temperature sensitive-mutations either with or without chemical mutagenesis (e.g., 5-fluorouracil), by passage in culture at “cold” temperatures. Such cold adaptation includes passage at temperatures between about 20° C. to about 32° C., and preferably between temperatures of about 22° C. to about 30° C., and most preferably between temperatures of about 24° C. and 28° C. The cold adaptation or attenuation may be performed by passage at increasingly reduced temperatures to introduce additional growth restriction mutations. The number of passages required to obtain safe, immunizing attenuated virus is dependent at least in part on the conditions employed. Periodic testing of the SARS virus culture for virulence and immunizing ability in animals (e.g., mouse, primate) can readily determine the parameters for a particular combination of tissue culture and temperature. The attenuated vaccine will typically be formulated in a dose of from about 10³ to 10⁶ PFU or TCID₅₀, or more for maximal efficacy.

Attenuated virus vaccines for SARS-CoV also are produced by creating virus chimeras comprising sequences derived from at least two different coronaviruses, one of which is a SARS-CoV. For example, a virus chimera is produced that comprises nonstructural protein encoding genes derived from a first coronavirus (e.g., murine, bovine, porcine, canine, feline, avian coronavirus) and one or more structural protein encoding genes (e.g., spike, E, M) from a SARS-CoV. Alternatively, the virus chimera may comprise sequences derived from a human coronavirus that is not a SARS-CoV (e.g., OC43, 229E) together with sequences from a SARS-CoV. Chimeric coronaviruses of the present invention are generated by a variety of methods, including for example allowing for natural RNA recombination in a eukaryotic (e.g., mammalian) cell that contains RNA from each of the parental coronaviruses (e.g., following infection) or by using standard molecular biology techniques known to those of skill in the art to engineer desired virus chimeras (or portions thereof) as cDNA clones, which may then be used to produce infectious virus (see for example, U.S. Pat. No. 6,593,111 B2; Yount et al., 2003, Proc. Natl. Acad. Sci. USA 100(22):12995-13000). An attenuated phenotype of the coronavirus chimeras described herein can be readily measured by one of skill in the art.

Attenuated viruses can be also generated by deleting one or more open reading frames (ORFs) that are not essential for viral replication. Preferably, these deletions occur in the structural region of the genome, such as ORF 3a, 3b, 6, 7a, 7b, 8a, 8b, 9b. See e.g., Haijema B J, Volders H, Rottier P J. J Virol. (2004) 78(8):3863-71; and de Haan, C. A., P. S. Masters, X. Shen, S. Weiss, and P. J. Rottier, “The group-specific murine coronavirus genes are not essential, but their deletion, by reverse genetics, is attenuating in the natural host.” Virology (2002) 296:177-189. Deletion of such regions within a coronavirus such as SARS can be achieved, for example, by reverse genetics or “targeted recombination” (See, e.g., Masters, P. S., “Reverse genetics of the largest RNA viruses”, Adv. Virus Res. (1999) 53:245-264.

Methods of purification of attenuated virus are known in the art and may include one or more of, for instance gradient centrifugation and chromatography. See Gregersen “Herstellung von Virussimpfstoffen aus Zellkulturen” Chapter 4.2 in Pharmazeutische Biotechnology (eds. O. Kayser and R H Mueller) Wissenschaftliche Verlagsgesellschaft, Stuttgart, 2000.

C. Split SARS Vaccines

The invention includes a composition comprising a split SARS virus formulation and methods for the manufacture thereof. This composition can be used as a prophylactic or therapeutic SARS virus vaccine.

Methods of splitting enveloped viruses are known in the art. Methods of splitting enveloped viruses are disclosed, for example, in WO 02/28422, incorporated herein by reference in its entirety, and specifically including the splitting agents and methods described therein. Methods of splitting influenza viruses are disclosed, for example, in WO 02/067983, WO 02/074336, and WO 01/21151, each of which is incorporated herein by reference in its entirety.

The splitting of the virus is carried out by disrupting or fragmenting whole virus, infectious (wild-type or attenuated) or non-infectious (for example inactivated), with a disrupting concentration of a splitting agent. The disruption results in a full or partial solubilisation of the virus proteins, altering the integrity of the virus.

Preferably, the splitting agent is a non-ionic or an ionic surfactant. Accordingly, the split SARS virus formulations of the invention may also comprise at least one non-ionic surfactant or detergent. Examples of splitting agents useful in the invention include: bile acids and derivatives thereof, non-ionic surfactants, alkylglycosides or alkylthioglycosides and derivatives thereof, acyl sugars, sulphobetaines, betains, polyoxyethylenealkylethers, N,N-dialkyl-Glucamides, Hecameg, alkylphenoxypolyethoxyethanols, quaternary ammonium compounds, sarcosyl, CTAB (cetyl trimethyl ammonium bromide) or Cetavlon.

Preferably, the ionic surfactant is a cationic detergent. Cationic detergents suitable for use in the invention include detergents comprising a compound of the following formula:

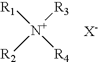

wherein

R₁ , R₂ and R₃ are the same or different and each signifies alkyl or aryl, or

R₁and R₂, together with the nitrogen atom to which these are attached form a 5- or 6-membered heterocyclic ring, and

R₃ signifies alkyl or aryl, or

R₁, R₂ and R₃ together with the nitrogen atom to which these are attached, signify a 5- or 6-membered heterocyclic ring, unsaturated at the nitrogen atom,

R₄ signfies alkyl or aryl, and

X signifies an anion.

Examples of such cationic detergents are cetyltrimethylammonium salts, such as ceytltrimethylammonium bromide (CTAB) and myristyltrimethylammonium salt.

Additional cationic detergents suitable for use in the invention include lipofectine, lipofectamine, and DOT-MA.

Non-ionic surfactants suitable for use in the invention include one or more selected from the group consisting of the octyl- or nonylphenoxy polyoxyethanols (for example the commercially available Triton series), polyoxyethylene sorbitan esters (Tween series) and polyoxyethylene ethers or esters of the general formula:
O(CH₂CH₂O)n-A-R

wherein n is 1-50, A is a bond or —C(O)—, R is C₁₋₅₀ alkyl or phenyl C₁₋₅₀ alkyl; and combinations of two or more of these.

The invention comprises a method of preparing a split SARS virus comprising contacting the SARS virus with a sufficient amount of splitting agent to disrupt the viral envelope. The loss of integrity after splitting renders the virus non-infectious. Once the disrupted viral envelope proteins are generally no longer associated with whole intact virions, other viral proteins are preferably fully or partially solubilized and are therefore not associated, or only in part associated, with whole intact virions after splitting.

The method of preparing a split SARS virus may further comprise removal of the splitting agents and some or most of the viral lipid material. The process may also include a number of different filtration and/or other separation steps such as ultracentrifugation, ultrafiltration, zonal centrifugation and chromatographic steps in a variety of combinations. The process may also optionally include an inactivation step (as described above) which may be carried out before or after the splitting. The splitting process may be carried out as a batch, continuous, or semi-continuous process.

Split SARS virus vaccines of the invention may include structual proteins, membrane fragments and membrane envelope proteins. Preferably, the split SARS virus preparations of the invention comprise at least half of the viral structural proteins.

One example of a method of preparing a split SARS virus formulation includes the following steps:

(i) propagation of the SARS virus in cell culture, such as MRC-5 cells (ATCC CCL-171), WI-38 cells (ATCC CCL-75), fetal rhesus kidney cells or vero cells (See the discussion in Section A, above, regarding culture of SARS virus);

(ii) harvesting of SARS virus-containing material from the cell culture;

(iii) clarification of the harvested material to remove non-SARS virus material;

(iv) concentration of the harvested SARS virus;

(v) separation of the whole SARS virus from non-virus material;

(vi) splitting of the whole SARS virus using a suitable splitting agent in a density gradient centrifugation step; and

(vii) filtration to remove undesired materials.

The above steps are preferably performed sequentially.

The clarification step is preferably performed by centrifugation at a moderate speed. Alternatively, a filtration step may be used for example with a 0.2 μm membrane.

The concentration step may preferably employ an adsorption method, for instance, using CaHPO₄. Alternatively, filtration may be used, for example ultrafiltration.

A further separation step may also be used in the method of the invention. This further separation step is preferably a zonal centrifugation separation, and may optionally use a sucrose gradient. The sucrose gradient may further comprise a preservative to prevent microbial growth.

The splitting step may also be performed in a sucrose gradient, wherein the sucrose gradient contains the splitting agent.

The method may further comprise a sterile filtration step, optionally at the end of the process. Preferably, there is an inactivation step prior to the final filtration step.

Methods of preparing split SARS virus formulations may further include treatment of the viral formulation with a DNA digesting enzyme. These enzymes may be used to reduce the level of host cell DNA in the viral purification process. DNA digesting enzymes for use in cell culture are known in the art and include, for example, Benzonase®.

Treatment of the SARS virus formulation with a DNA digesting enzyme may occur at any time in the purification and splitting process. Preferably, however, the SARS virus formulation is treated with a DNA digesting enzyme prior to use of a detergent. Still more preferably, the SARS virus formulation is treated with a DNA digesting enzyme, such as Benzonas, prior to treatment with a cationic detergent, such as CTAB.

Methods of purification of split virus are known in the art. See J P Gregersen “Herstellung von Virussimpfstoffen aus Zellkulturen” Chapter 4.2 in Pharmazeutische Biotecnologie (eds. O. Kayser and R H Mueller) Wissenschaftliche Verlagsgesellschaft, Stuttgart, 2000.

The invention includes a split vaccine composition comprising a prophylactically effective amount of SARS viral antigen, preferably spike or an immunogenic fragment thereof. The SARS viral antigen is preferably present in a concentration amount of 0.1 to 50 μg antigen/dose, more preferably 0.3 to 30 μg antigen/dose. Still more preferably, the antigen is about 15 μg/dose.

In one embodiment, a lower concentration of SARS viral antigen is used in split vaccine compositions of the invention. Such lower concentration vaccines may optionally comprise an adjuvant to boost the host immune response to the antigen. In such a “low dose” vaccine, the SARS viral antigen is preferably present in a concentration of less than 15 μg antigen/dose, (i.e., less than 10, 7.5, 5 or 3 μg antigen/dose.

D. Subunit SARS Vaccines

The invention includes a composition comprising an isolated or purified SARS viral antigen or a derivative thereof. The composition may further comprise one or more adjuvants.

SARS viral antigens can be isolated or purified from a SARS virus grown in cell culture. Alternatively, SARS viral antigens can be recombinantly produced by methods known in the art.

The SARS viral antigens used in the invention can be produced in a variety of different expression systems which are known in the art; for example those used with mammalian cells, baculoviruses, bacteria, and yeast. Such expression systems will typically use polynucleotides encoding the viral antigens of the invention. Such sequences can be obtained using standard techniques of molecular biology, including translating the amino acid sequences listed herein. Accordingly, the invention includes polynucleotides encoding for the viral antigens of the invention. In addition, the viral antigens of the invention can be produced (at least in part, preferably in whole) via synthetic chemistry methods.

Insect cell expression systems, such as baculovirus systems, are known to those of skill in the art and described in, e.g., Summers and Smith, Texas Agricultural Experiment Station Bulletin No. 1555 (1987). Materials and methods for baculovirus/insert cell expression systems are commercially available in kit form from, inter alia, Invitrogen, San Diego Calif. Similarly, bacterial and mammalian cell expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (e.g., Hep G2), Madin-Darby bovine kidney (“MDBK”) cells, as well as others. Mammalian sources of cells include, but are not limited to, human or non-human primate (e.g., MRC-5 (ATCC CCL-171), WI-38 (ATCC CCL-75), fetal rhesus lung cells (ATCC CL-160), human embryonic kidney cells (293 cells, typically transformed by sheared adenovirus type 5 DNA), VERO cells from monkey kidneys), horse, cow (e.g., MDBK cells), sheep, dog (e.g., MDCK cells from dog kidneys, ATCC CCL34 MDCK (NBL2) or MDCK 33016, deposit number DSM ACC 2219 as described in WO 97/37001), cat, and rodent (e.g., hamster cells such as BHK21-F, HKCC cells, or Chinese hamster ovary cells (CHO cells)), and may be obtained from a wide variety of developmental stages, including for example, adult, neonatal, fetal, and embryo.

Similarly, bacterial hosts such as E. coli, Bacillus subtilis, and Streptococcus spp., will find use with the present expression constructs. Yeast hosts useful in the present invention include, inter alia, Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Nucleic acid molecules comprising nucleotide sequences of the viral antigens or antibodies of the invention can be stably integrated into a host cell genome or maintained on a stable episomal element in a suitable host cell using various gene delivery techniques well known in the art. See., e.g., U.S. Pat. No. 5,399,346.

Depending on the expression system and host selected, the molecules are produced by growing host cells transformed by an expression vector under conditions whereby the protein is expressed. The expressed protein is then isolated from the host cells and purified. If the expression system secretes the protein into growth media, the product can be purified directly from the media. If it is not secreted, it can be isolated from cell lysates. The selection of the appropriate growth conditions and recovery methods are within the skill of the art.

The invention includes a composition comprising an isolated or purified SARS viral antigen or a derivative thereof. The invention also includes a composition comprising at least two isolated or purified SARS viral antigens or derivatives thereof, which have been co-purified or purified separately and then combined. In one embodiment, the SARS viral antigen is a spike (S) protein. In yet another embodiment, the SARS viral antigen is a nucleocapsid (N) protein, a membrane (M) glycoprotein, or an envelope (E) protein. Preferably, the SARS viral antigen is present in the composition in a purity greater than 75% (e.g., 78%, 80%, 82%, 85%, 88%, 90%, 92%, 95%, 98%).

The invention includes a vaccine composition comprising a prophylactically effective amount of SARS viral antigen, preferably spike or an immunogenic fragment thereof. The SARS viral antigen is preferably present in a concentration amount of 0.1 to 50 μg antigen/dose, more preferably 0.3 to 30 μg antigen/dose. Still more preferably, the antigen is about 15 μg/dose.

In one embodiment, a lower concentration of SARS viral antigen is used in vaccine compositions of the invention. Such lower concentration vaccines may optionally comprise an adjuvant to boost the host immune response to the antigen. In such a “low dose” vaccine, the SARS viral antigen is preferably present in a concentration of less than 15 μg antigen/dose, (i.e., less than 10, 7.5, 5 or 3 μg antigen/dose.

The following example illustrates a method of preparing a SARS virus spike (S) protein subunit vaccine.

SARS virus S antigen may be isolated and purified from a variety of sources and using a variety of methods, including, but not limited to, S antigen expressed in cultured eukaryotic cells (e.g., mammalian cells, such as VERO, CHO) or bacteria (e.g., E. coli). Expression of may be achieved by a variety of means, such as, for example, from SARS virus infected cell culture or cell culture supernatants, from cultured cells stably transformed with a DNA expression cassette encoding the SARS virus S protein (e.g., RNA polymerase II promoter operably linked to a SARS virus S gene), or from cultured cells infected with a replication-competent or replication-incompetent virus-based expression vector (e.g., adenovirus vector, poxvirus vector, alphavirus vector, retrovirus vector) encoding the SARS virus S protein, as a means to eliminate the need to work with infectious SARS virus.

1. Subunit SARS Vaccines Produced from SARS Virus Cultures

The SARS virus may be grown in cultured mammalian celle, such as VERO cells, then separated from the cultured cells. A SARS viral antigen, such as the S protein, can then be solubilized and separated from the SARS virus, and further isolated and purified.

In one example, the SARS virus may be produced as described in the Inactivated SARS vaccine examples, then the desired SARS antigen, such as spike protein, may be further purified from the end product using techniques known in the art.

In another example, a SARS subunit vaccine may be produced as follows. SARS virus may be produced using a desired mammalian cell line on microcarrier beads in large, controlled fermentors. For example, vaccine quality African Green Monkey kidney cells (VERO cells) at a concentration of 10⁵ cells/mL are added to 60 to 75 L of CMRL 1969 media, pH 7.2, in a 150 L bioreactor containing 360 g of Cytodex-1 microcarrier beads and stirred for 2 hours. Additional CMRL 1969 is added to give a total volume of 150 L. Fetal bovine serum (FBS) is added to a final concentration of 3.5%. Glucose is added to a final concentration of 3.0 g/L and glutamine is added to a final concentration of 0.6 g/L. Dissolved oxygen, pH, agitation and temperature are controlled, and cell growth, glucose, lactate and glutamine levels are monitored. When cells are in logarithmic phases usually on days 3 to 4 reached a density of about 1.0-2.5×10⁶ cells/mL, the culture medium is drained from the fermentor and 120 L of CMRL 1969, pH 7.2 (no FBS) is added and the culture stirred for 10 minutes. The draining and filling of the fermentor is usually repeated once but could be repeated up to three times. After washing the cells, the fermentor is drained and 50 L of CMRL 1969 containing 0.1% (v/v) FBS is added. The SARS virus inoculum is added at a multiplicity of infection (m.o.i.) of 0.001 to 0.01. Trypsin may be added to promote efficient infection. Additional CMRL 1969 with 0.1% FBS is added to give a final volume of 150 L. Incubation is continued at 34 C. One viral harvest is obtained from a single fermentor lot, typically at 2-7 days post-infection. Multiple harvests from a single fermentation may also be obtained.

The isolation and purification of S protein may be effected by a variety of means, as described below. For example, collecting S protein-containing flow-through from ion exchange chromatography of solubilized SARS virus envelope proteins; loading the flow through onto a hydroxyapatite matrix, and selectively eluting the S protein from the hydroxyapatite matrix. The selectively eluted S protein may be further concentrated by tangential flow ultrafiltration.

Alternatively, the isolation and purification may be effected by collecting S protein-containing flow-through from ion exchange chromatography of the solubilized SARS virus envelope proteins; loading the flow through onto a hydroxyapatite matrix and collecting an S protein-containing flow through, selectively removing detergent used in the solubilization step from the hydroxyapatite matrix flow through to provide isolated and purified S protein. The isolated and purified S protein may be subsequently concentrated by tangential flow ultrafiltration

Nucleic acid contaminants may be removed from the isolated and purified S protein by treatment with a nucleic acid degrading agent as described above in the Inactivation section. Preferably, the nucleic acid degrading agent is a nuclease, such as for example, Benzonase.

The isolated and purified S protein may be applied to a gel filtration medium and the S protein subsequently collected therefrom to separate the S protein from contaminants of other molecular weights.

Alternatively, the isolation and purification may be effected by loading S protein on a first ion-exchange medium while permitting contaminants to pass through the medium, eluting the S protein from the first ion-exchange medium, to separate the S protein from contaminants of other molecular weights. The eluted S protein is applied to a second ion-exchange medium while allowing contaminants to pass through the second ion-exchange medium. The S protein is subsequently eluted therefrom, to provide the isolated and purified S protein. The eluted S protein may be concentrated by tangential flow ultrafiltration.

Alternatively, substantially pure SARS virus S protein suitable for use as an immunogen in a subunit vaccine formulation may be prepared from infected cell lysates, such as for example using a non-denaturing detergent buffer containing 1% Triton X-100 and deoxycholate to lyse infected cells. The cell lysates are clarified by centrifugation and S protein is purified from the cell lysates by immunoaffinity purification. A monoclonal antibody against the S protein is generated and coupled to beads and a column is constructed with those beads. SARS-infected cell lysates are applied to the column, and the column is washed with PBS containing 0.1% Triton X-100. Protein bound to the column is eluted with 0.1M glycine, pH 2.5, 0.1% Triton X-100. Elution samples are buffered, such as for example, with Tris, and analyzed for the presence of protein. Fractions containing the protein are pooled and dialyzed against PBS

As discussed above, the present invention includes isolated and purified S protein of SARS virus. In one example, the virus is grown on a vaccine quality cell line, such as VERO cells, and the grown virus is harvested. The virus harvest is filtered and then concentrated typically using tangential flow ultrafiltration using a membrane of desired molecular weight cut-off and diafiltered. The virus harvest concentrate may be centrifuged and the supernatant discarded. The pellet from the centrifugation then is detergent extracted to solubilize the S protein, for example, by resuspending the pellet to the original harvest concentrate volume in an extraction buffer containing a detergent such as a non-ionic detergent including TRITON X-100.

Following centrifugation to remove non-soluble proteins, the S protein extract is purified by chromatographic procedures. The extract may first be applied to an ion exchange chromatography column such as a TMAE-fractogel or S-fractogel column equilibrated to permit the S protein to flow through while impurities are retained on the column.

Next, the flow through may be loaded onto a hydroxyapatite column, equilibrated to permit binding of the S protein to the matrix and to permit contaminants to pass from the column. The bound S protein is then eluted from the column by a suitable elutant. The resulting purified solution of S protein may be further processed to increase its purity. The eluate first may be concentrated by tangential flow ultrafiltration using a membrane of desired molecular weight cut-off. The filtrate may be contacted with a polyethylene glycol of desired molecular weight, for example, about 6000 to 8000, to precipitate the protein. Following centrifugation and discard of the supernatant, the pellet may be resuspended in PBS and dialyzed to remove the polyethylene glycol. Finally, the dialyzed solution of S protein may be sterile filtered. The sterile filtered solution may be adsorbed onto alum. The polyethylene glycol precipitation and resuspension purification step may be effected at an earlier stage of the purification operation, if desired.

Alternatively, SARS virus is recovered following growth and harvesting of the virus, and a concentrate obtained such as, for example using PEG precipitation or tangential flow filtration. The virus is contacted with detergent to solubilize the S proteins. Following centrifugation, the supernatant is recovered to further purification of the S protein and the non-soluble proteins discarded.

The supernatant is applied to an ion exchange chromatography column, such as a TMAE-fractogel or S-fractogel column, suitably equilibrated to permit retention of the S protein on the column. The S protein is eluted from the ion-exchange column under suitable conditions. The eluate then may be passed through a gel filtration column, such as a Sephacryl S-300 column, to separate the S protein from contaminants of other molecular weights. A hydroxyapatite column may be employed in place of the Sephacryl column.

The S protein may be eluted from the column to provide a purified solution of S protein. The eluate may be concentrated by tangential flow ultrafiltration using a membrane of desired molecular weight cut-off. The concentrated S protein solution then may be sterile filtered.

Alternatively, viral harvests may be concentrated by ultrafiltration and the concentrated viral harvests may be subjected to an initial purification step, for example, by gel filtration chromatography, polyethylene glycol precipitation or Cellufine sulfate chromatography. The purified virus may then be detergent extracted to solubilize the S protein. Following solubilization of the S protein, the supernatant may be loaded onto an ion-exchange column such as Cellufine sulfate chromatography column equilibrated to permit the protein to bind to the column while permitting contaminants to flow through. Similarly, a TMAE-fractogel or S-fractogel column may be used in place of the Cellufine sulfate column. The two columns also may be combined in sequential purification steps. The S protein is eluted from the columns to provide a purified solution of the protein. This solution may be concentrated by tangential flow ultrafiltration using a membrane of desired molecular weight cut-off and diafiltered.

Specifically, in one method of S protein purification, the virus harvest concentrate is centrifuged at 28,000×g for 30 minutes at 4 C. The supernatant is discarded and the pellet resuspended in extraction buffer consisting of 10 mM Tris-HCl, pH 7.0, 150 mM NaCl, 2% (w/v) Triton X-100 to the original harvest concentrate volume. Pefabloc is added to a final concentration of 5 mM. The suspension is stirred at room temperature for 30 minutes. The supernatant, containing the soluble S protein, is clarified by centrifugation at 28,000×g for 30 minutes at 4 C. A TMAE--Fractogel column is equilibrated with 10 mM Tris-HCl, pH 7.0, 150 mM NaCl containing 0.02% Triton X-100. The Triton X-100 supernatant, containing the soluble S protein, is loaded directly onto the TRAE-Fractogel column. The total volume added plus 2 bed volumes of 10 mM Tris-HCl, pH 7.0, 150 mM NaCl containing 0.02% Triton X-100 are collected. The TMAE-Fractogel flow-through containing S protein is diluted 3-fold with 10 mM Tris-HCl, pH 7.0, containing 0.02% Triton X-100.

An hydroxyapatite column is equilibrated with 10 mM Tris-HCl, pH 7.0, 50 mM NaCl, 0.02% Triton X-100. After loading the TMAE flow-through, the column is washed with 2 column volumes of 10 mM Tris-HCl, pH 7.0, 50 mM NaCl, 0.02% Triton X-100 followed by 4 column volumes of 5 mM sodium phosphate, pH 7.0, 1M NaCl, 0.02% Triton X-100. The proteins are eluted with 4 column volumes of 20 mM sodium phosphate, pH 7.0, 1M NaCl, 0.02% Triton X-100. Fractions are collected based on A280 and the protein content and antigen concentrations are measured. The purified S protein is ultrafiltered by tangential flow ultrafiltration using a 300 kDa NMWL membrane.

2. Recombinant Production of Subunit SARS Vaccines

As discussed above, SARS virus proteins may be produced by recombinant expression. Host cells suitable for recombinant expression include bacterial, mammalian, insect, yeast, etc. Recombinant expression may be used to produce a full length SARS protein, a fragment thereof, or a fusion therewith.

Fusion peptides may be used to facilitate the expression and purification of the recombinant SARS protein. For example, recombinant production of the SARS polypeptides can be facilitated by the addition a tag protein to the SARS antigen to be expressed as a fusion protein comprising the tag protein and the SARS antigen. Such tag proteins can facilitate purification, detection and stability of the expressed protein. Tag proteins suitable for use in the invention include a polyarginine tag (Arg-tag), polyhistidine tag (His-tag), FLAG-tag, Strep-tag, c-myc-tag, S-tag, calmodulin-binding peptide, cellulose-binding domain, SBP-tag,, chitin-binding domain, glutathione S-transferase-tag (GST), maltose-binding protein, transcription termination anti-terminiantion factor (NusA), E. coli thioredoxin (TrxA) and protein disulfide isomerase I (DsbA). Preferred tag proteins include His-tag and GST. A full discussion on the use of tag proteins can be found at Terpe et al., “Overview of tag protein fusions: from molecular and biochemical fumdamentals to commercial systems”, Appl Microbiol Biotechnol (2003) 60:523-533.

After purification, the tag proteins may optionally be removed from the expressed fusion protein, i.e., by specifically tailored enzymatic treatments known in the art. Commonly used proteases include enterokinase, tobacco etch virus (TEV), thrombin, and factor Xₐ.

Accordingly, the invention further includes a SARS virus subunit vaccine comprising a fusion protein. Preferably, the fusion protein comprises a first amino acid sequence encoded by a SARS virus polynucleotide sequence. SARS virus polynucleotide sequences which may encode said first amino acid sequence include one or more of the SARS virus polynucleotide sequences identified in this application and fragments thereof.

The fusion protein may comprise an amino acid sequence of a SARS virus protein or a fragment thereof. Said SARS virus protein may be selected from one or more of the group consisting of the following SARS virus proteins: P28, P65, Nsp1, Nsp2 (3CL protease), Nsp3, Nsp3, Nsp4, Nsp 5, Nsp6, Nsp 7, Nsp 8, Nsp 9 (RNA polymerase), Nsp 10 (helicase), Nsp 11, Nsp 12, Nsp 13, Spike, Orf 3, Orf 4, Envelope, Matrix, Orf7, Orf8, Orf9, Orf10, Orf11, Nucleocapsid and Orf13.

In one embodiment, the fusion protein comprises a first amino acid sequence comprising a SARS virus antigen or a fragment thereof. Said SARS virus amino acid sequence may comprise one or more of the T-epitope sequences identified above.

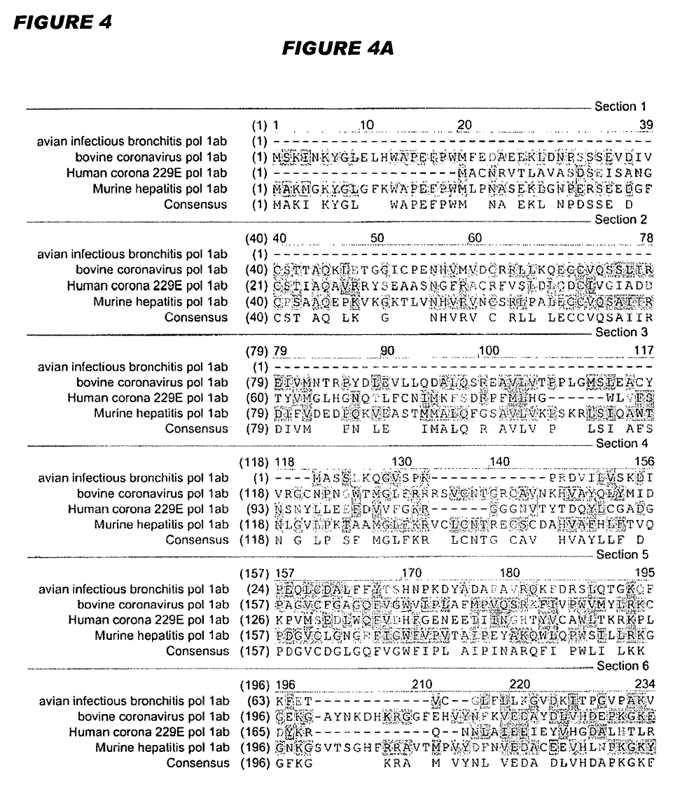

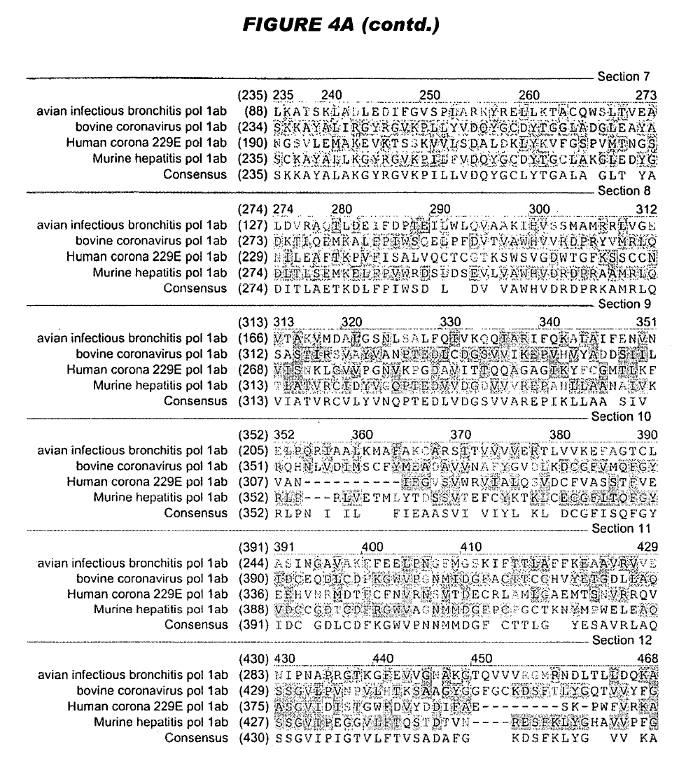

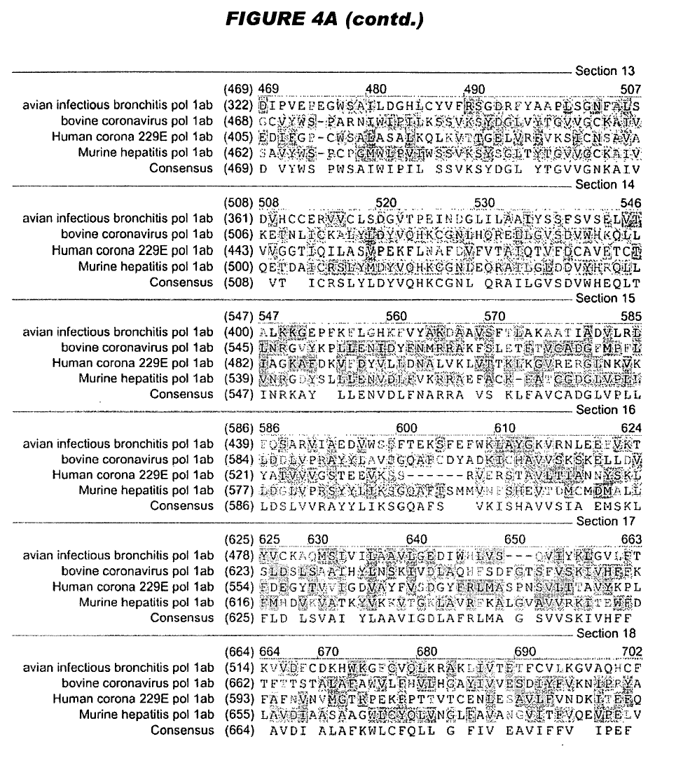

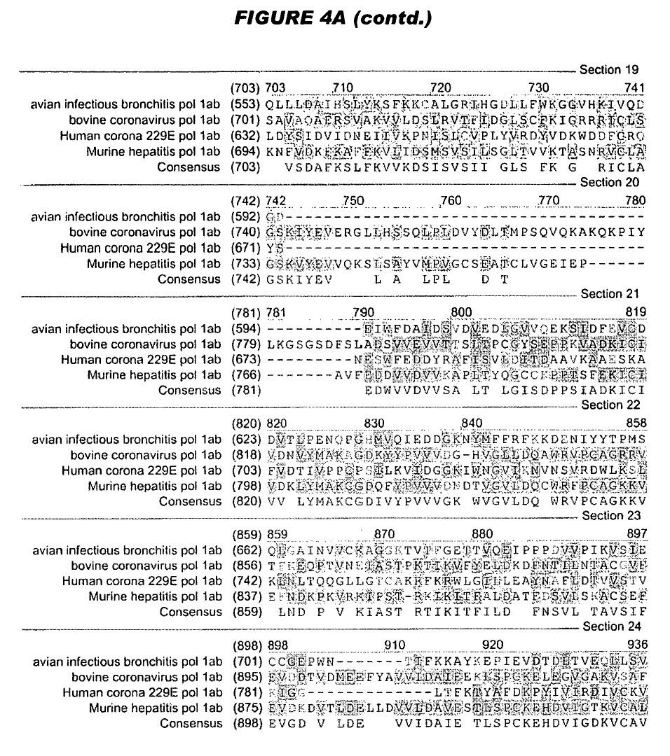

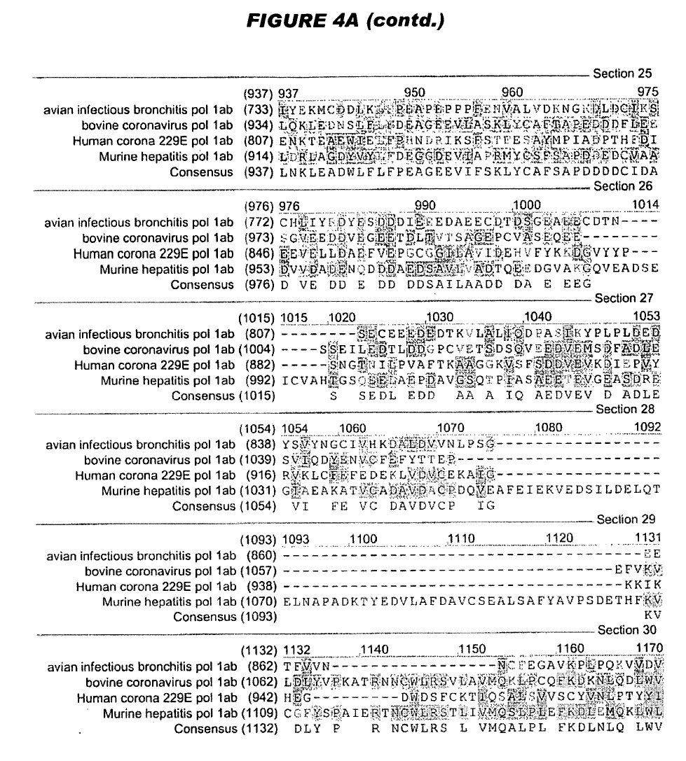

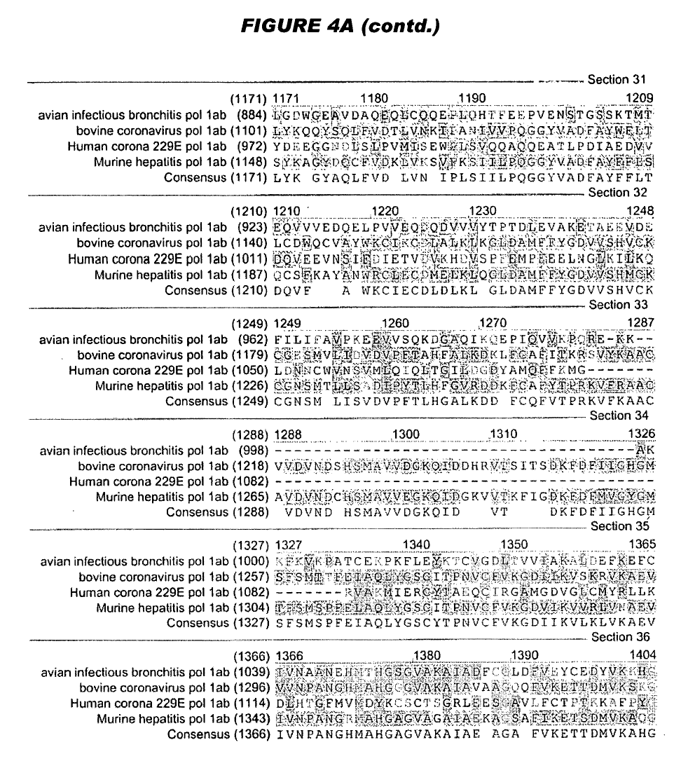

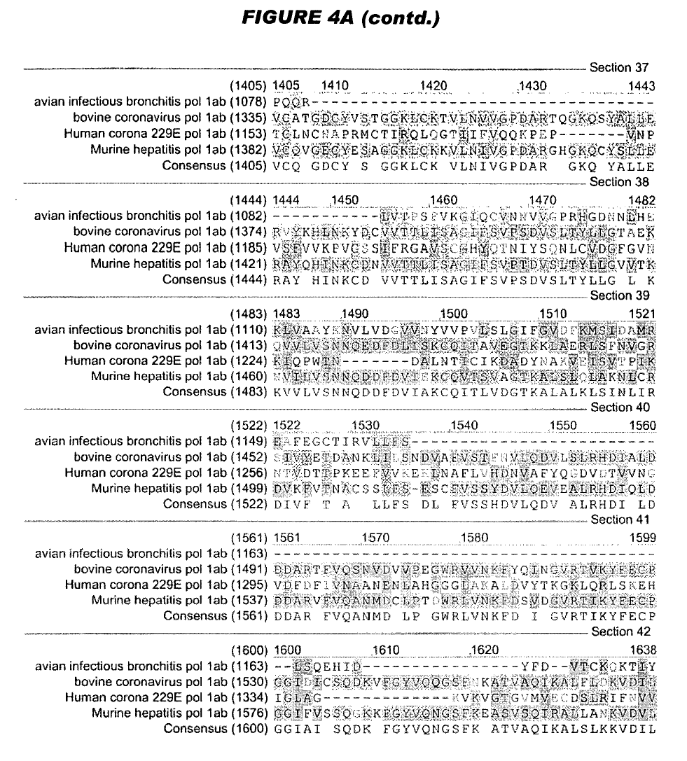

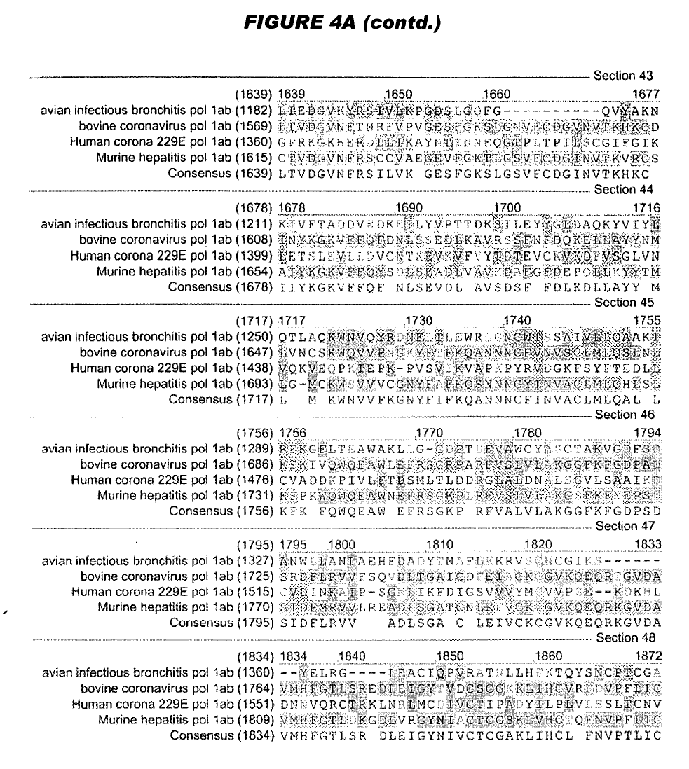

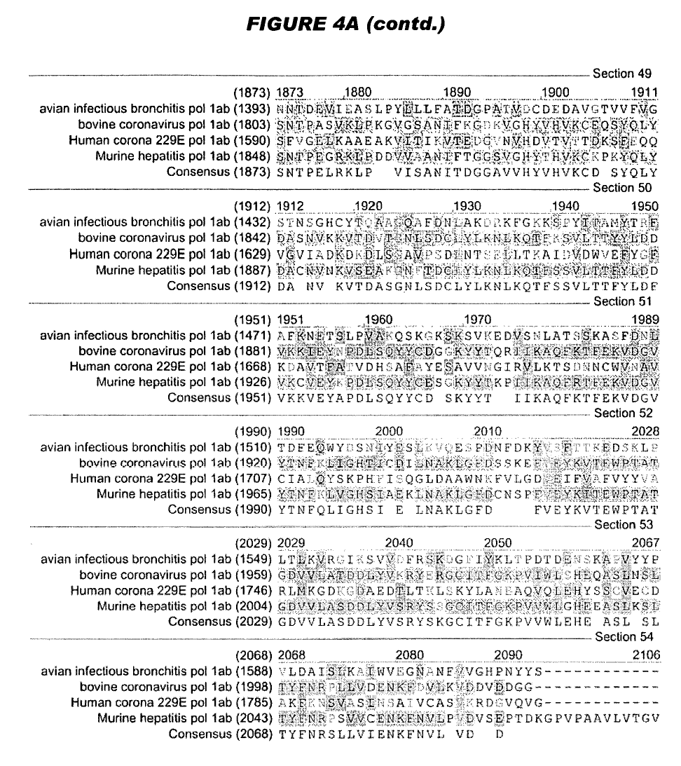

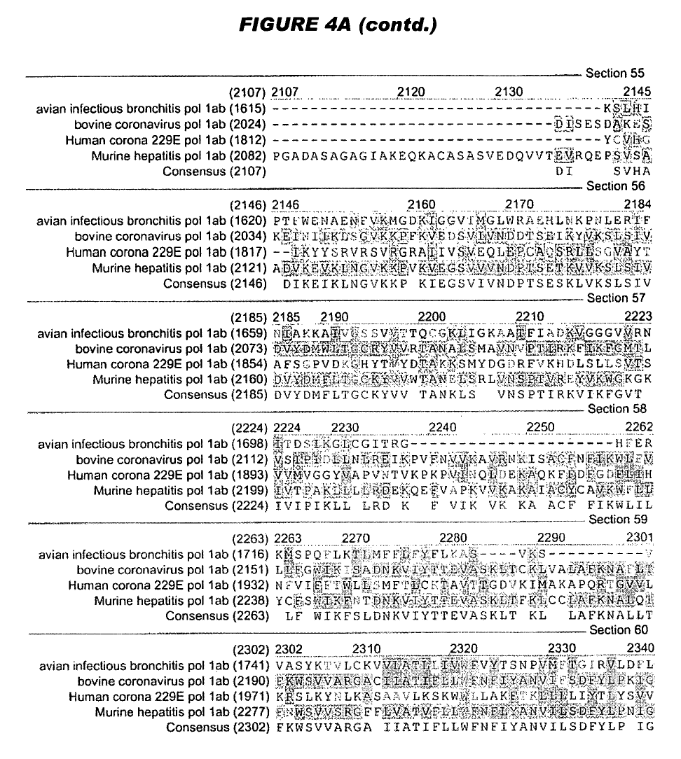

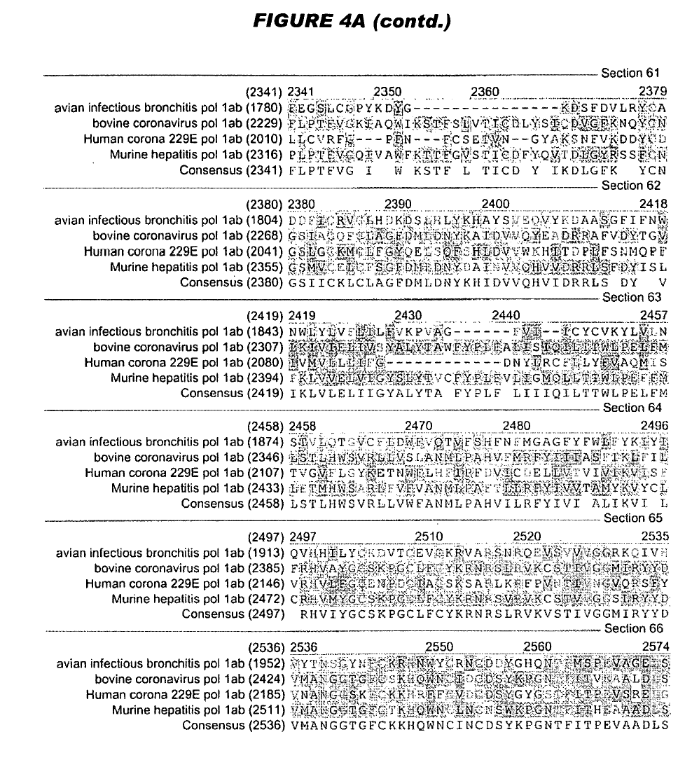

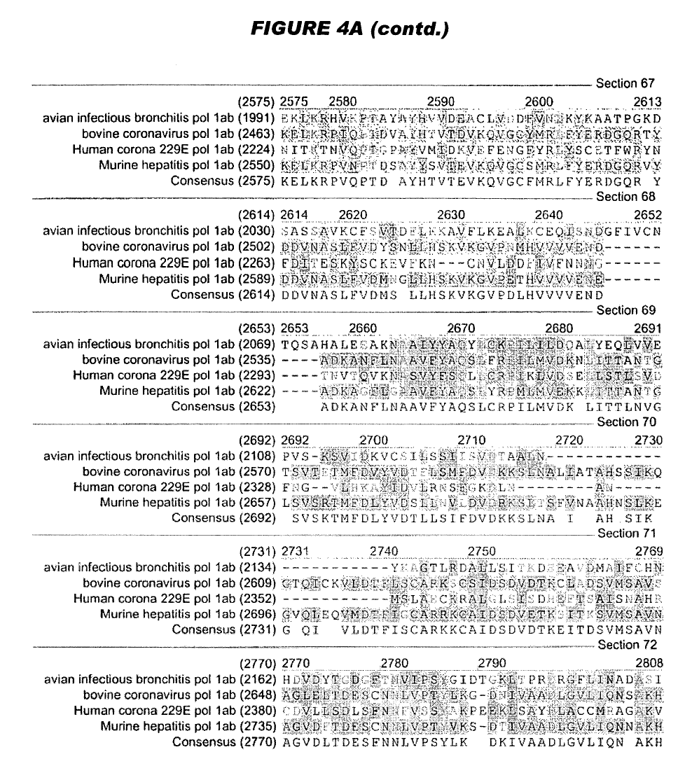

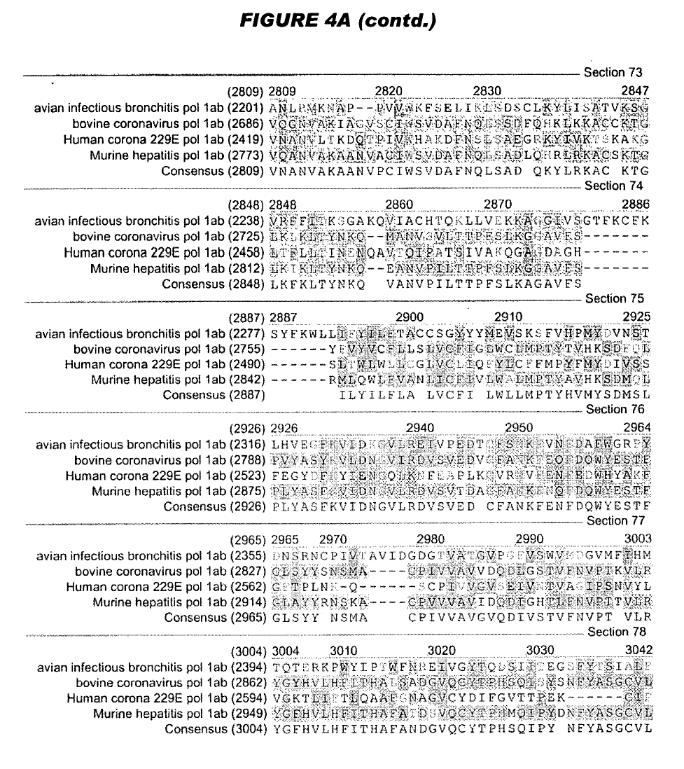

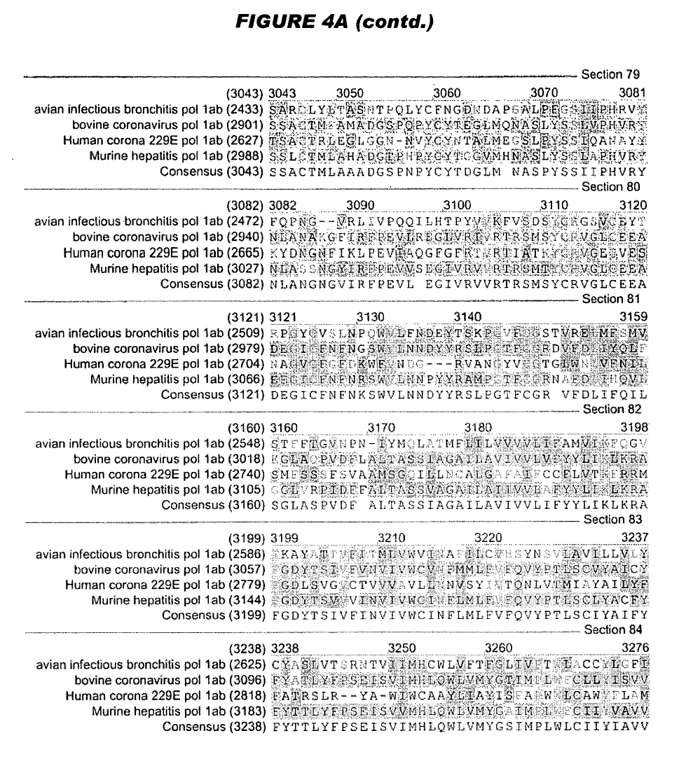

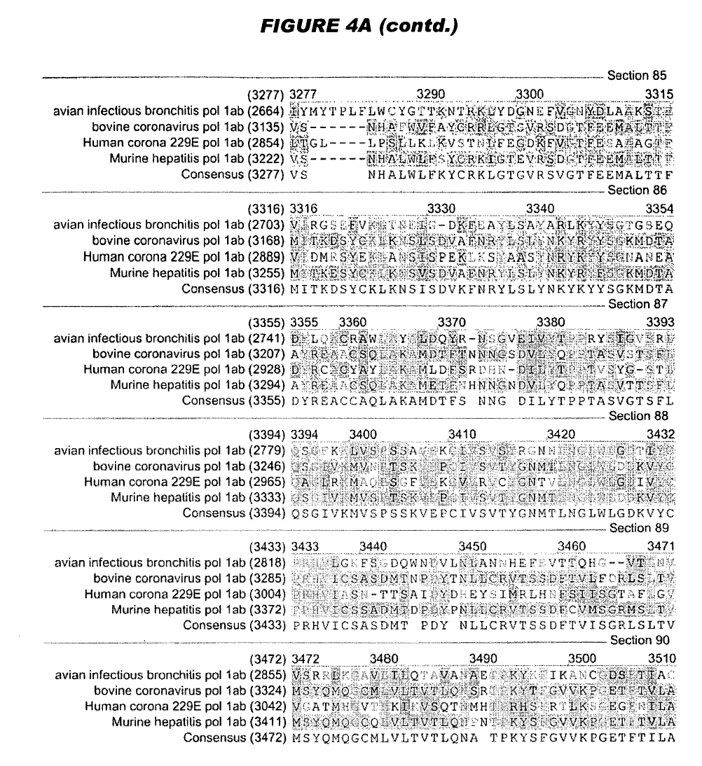

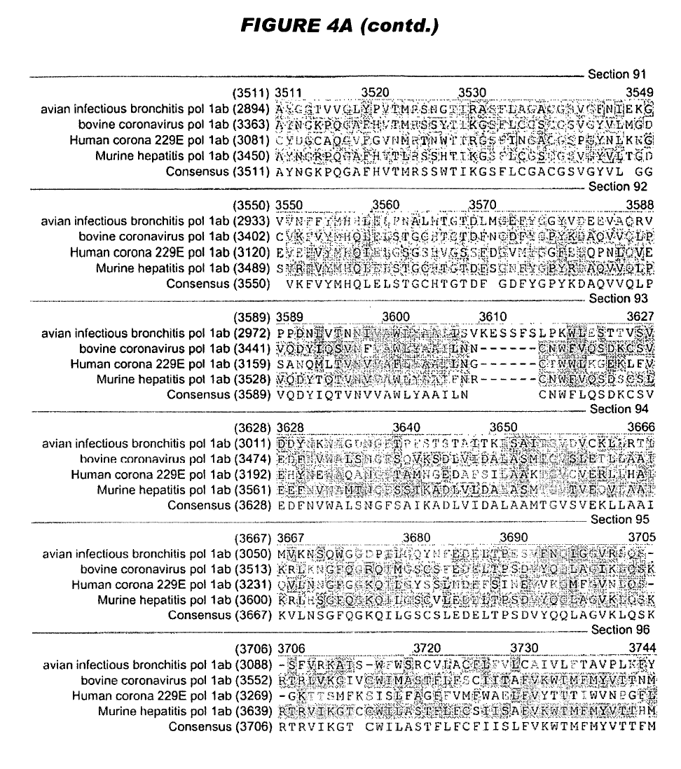

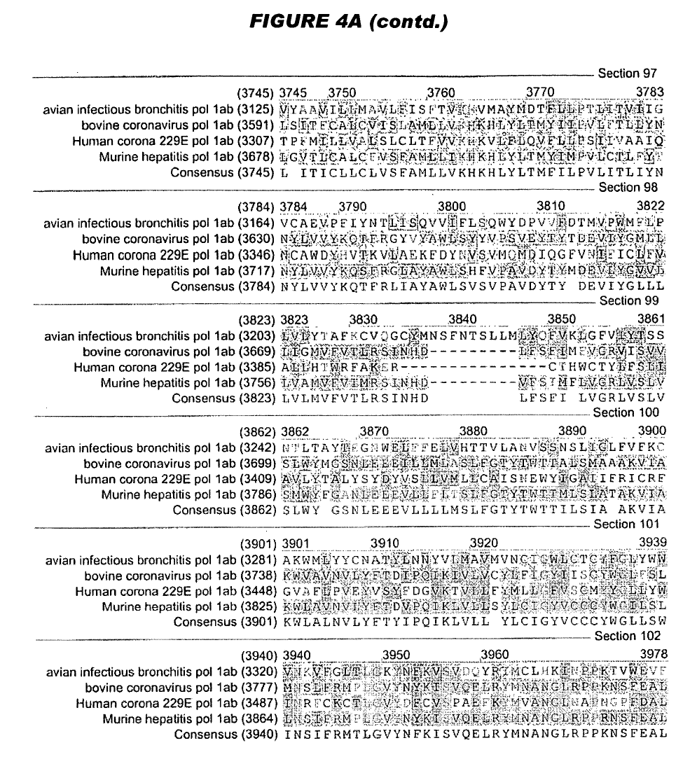

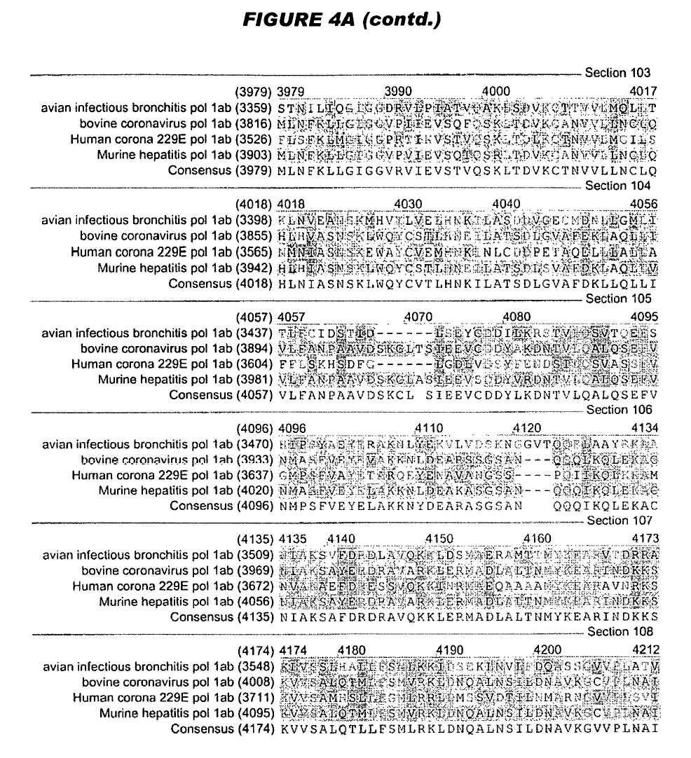

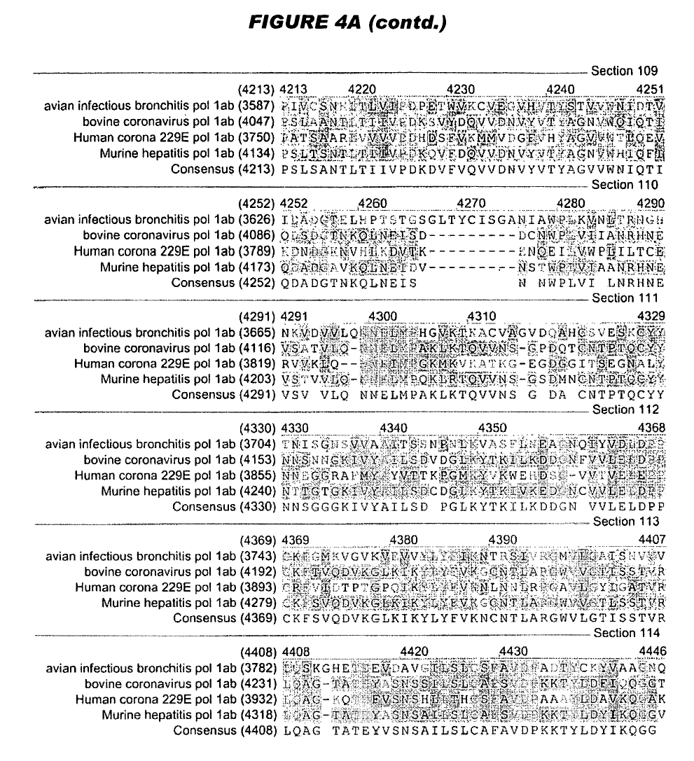

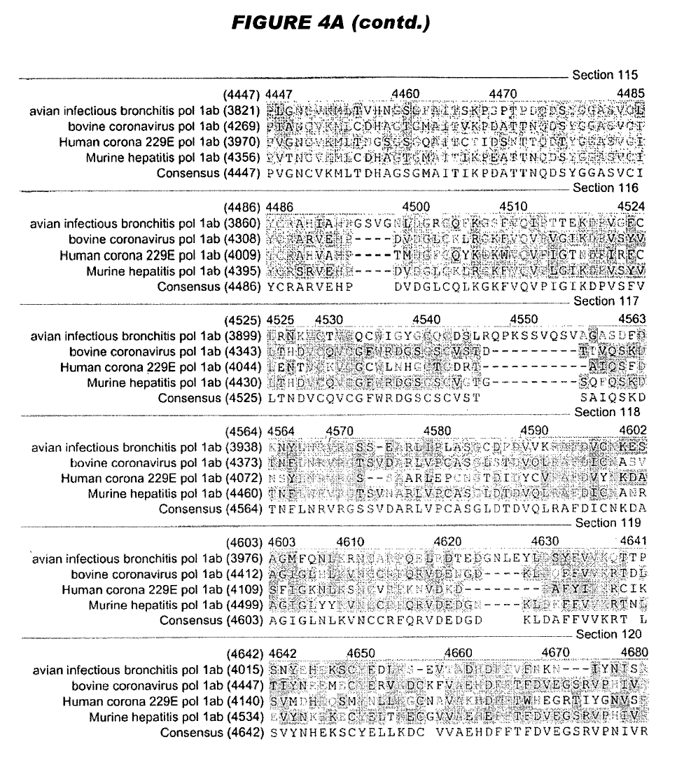

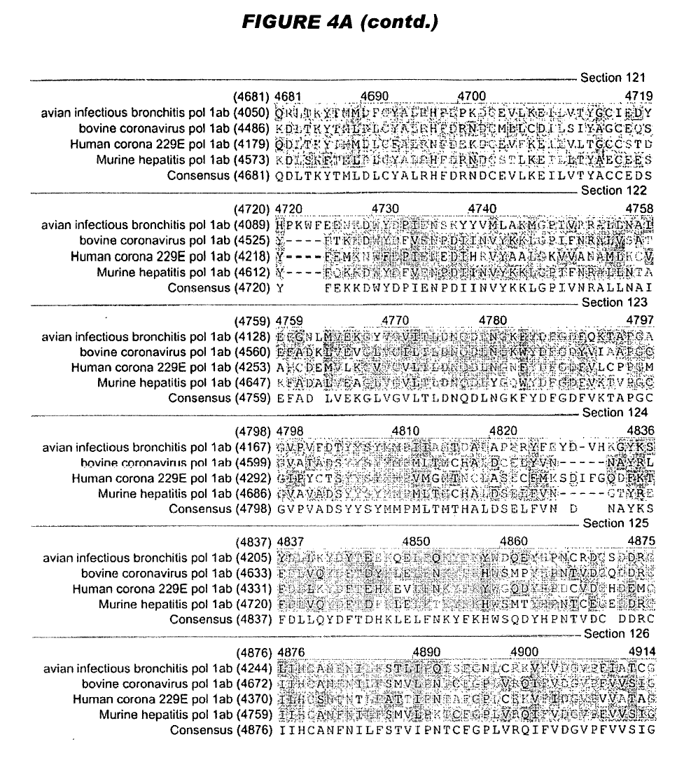

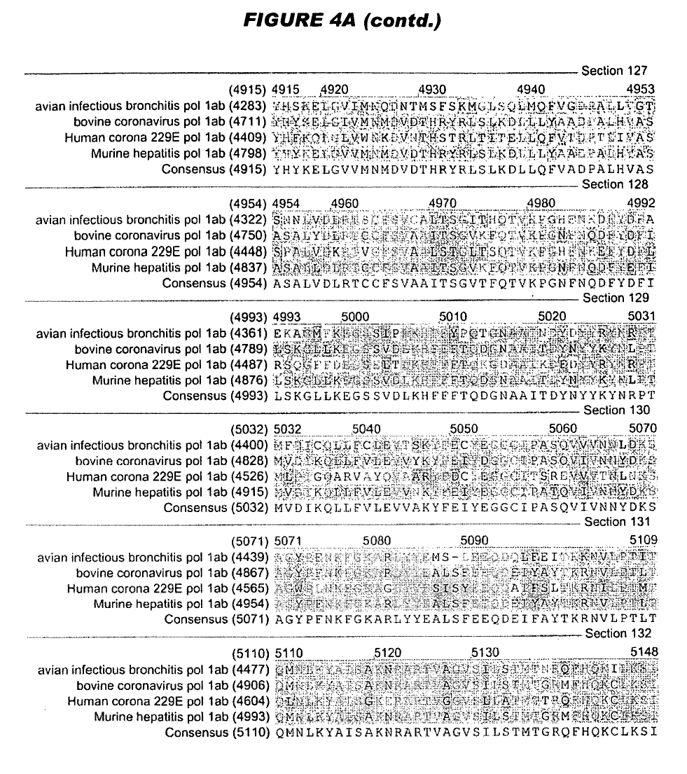

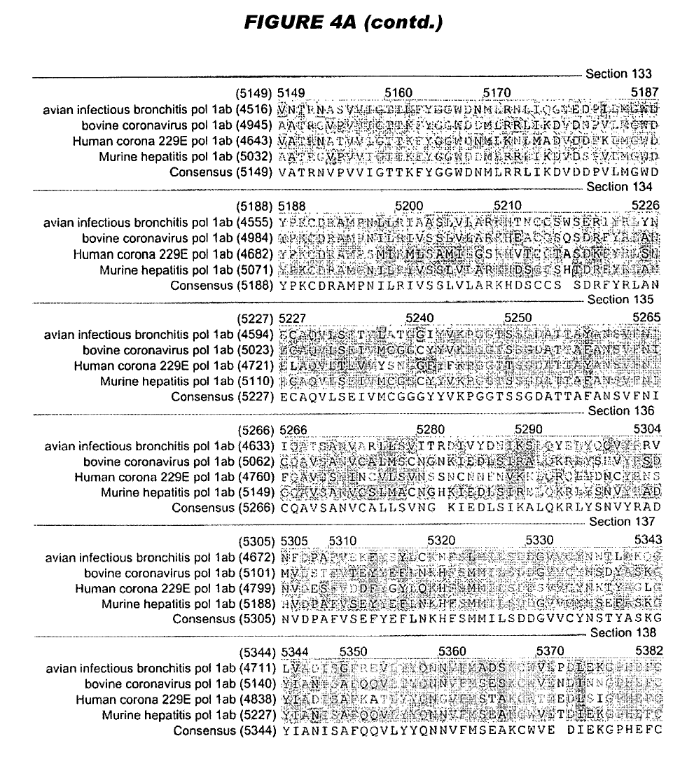

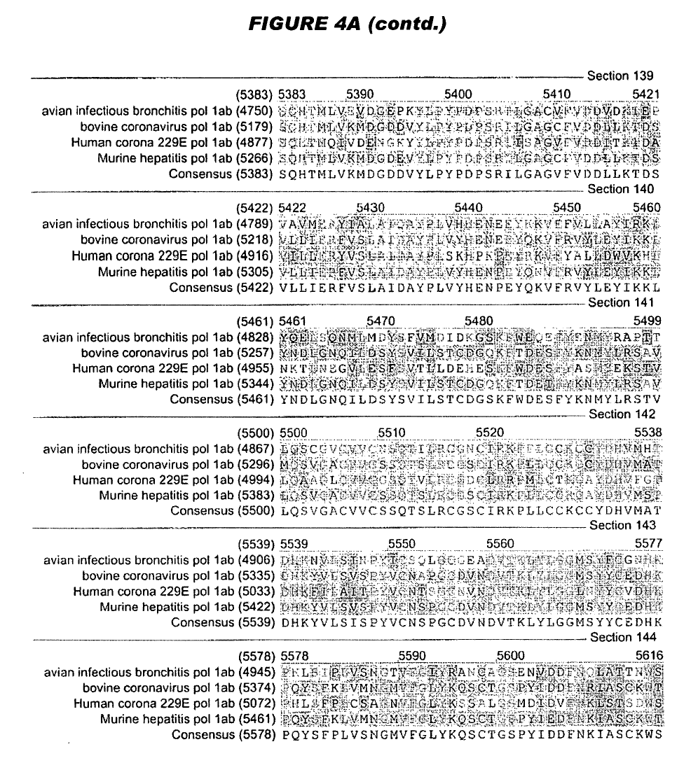

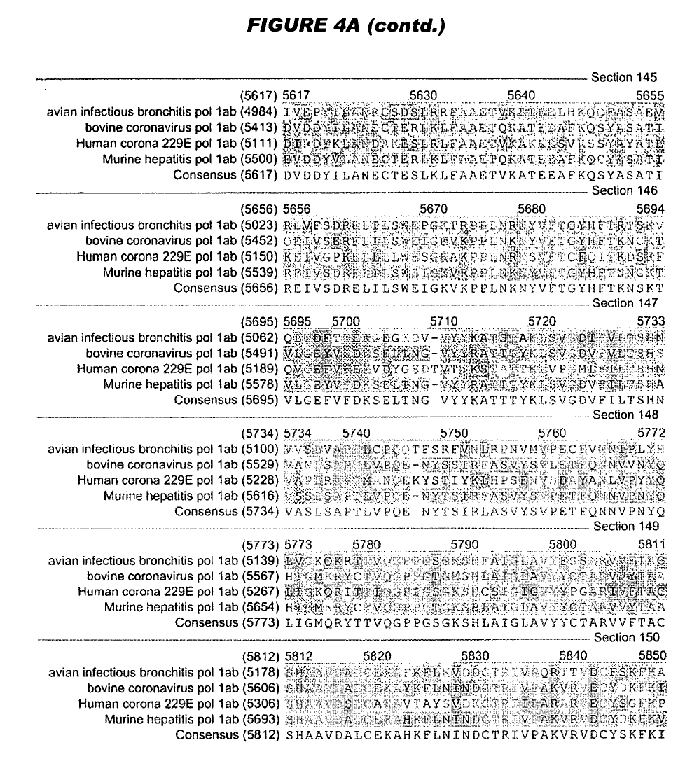

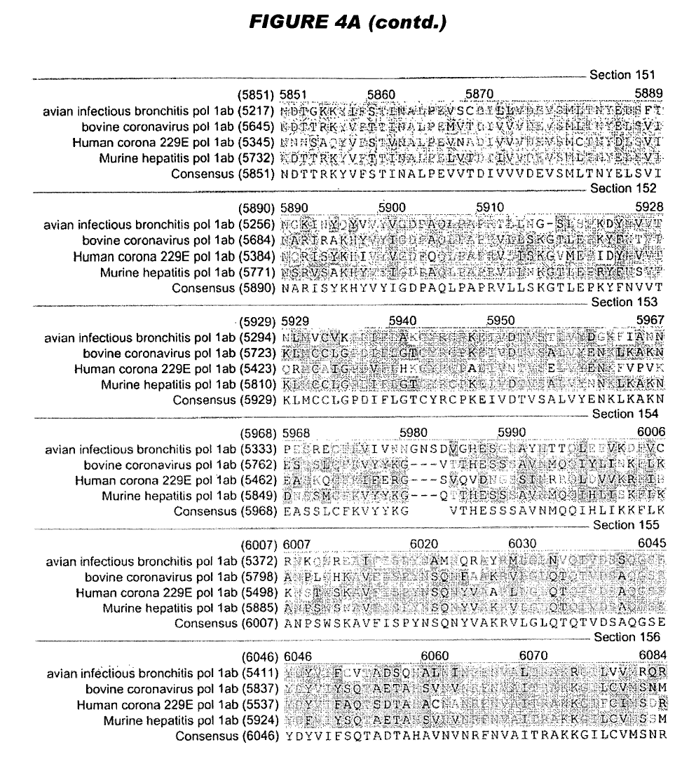

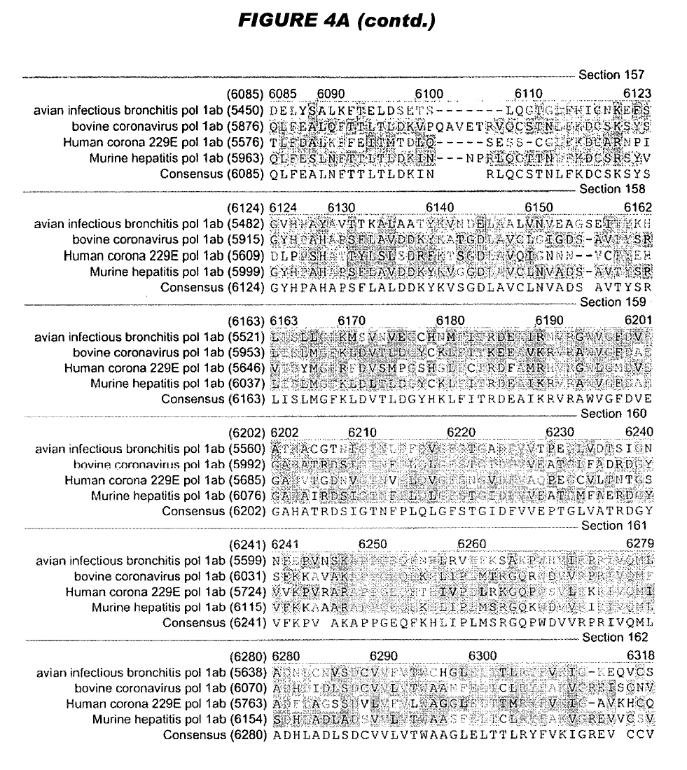

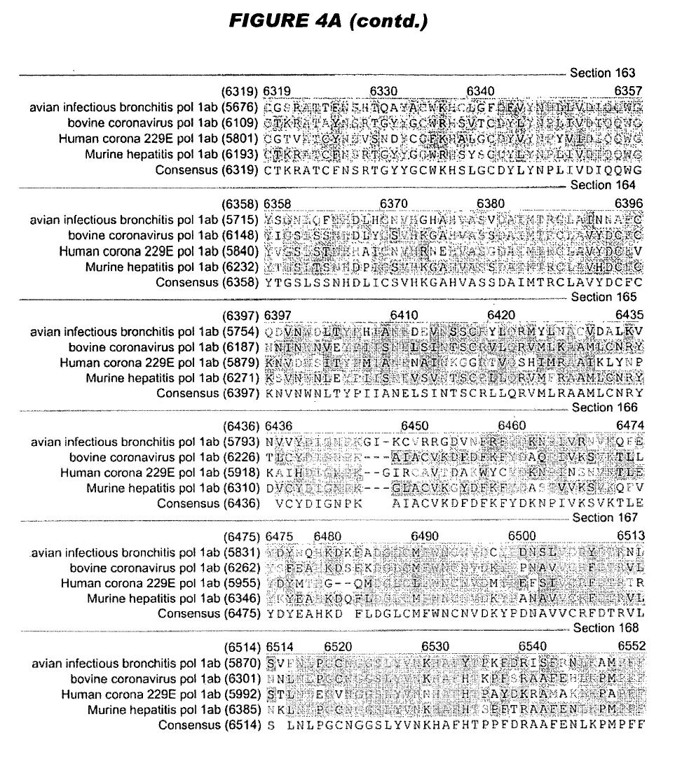

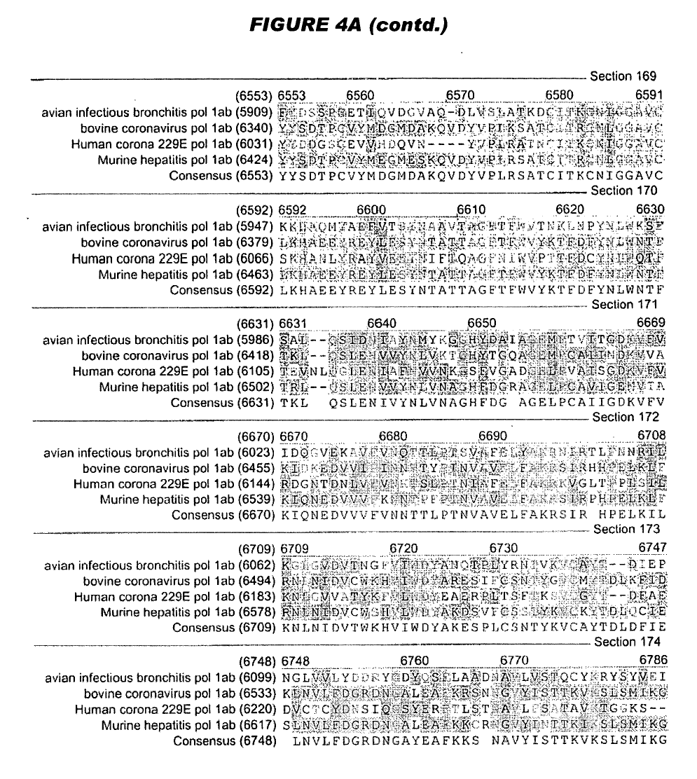

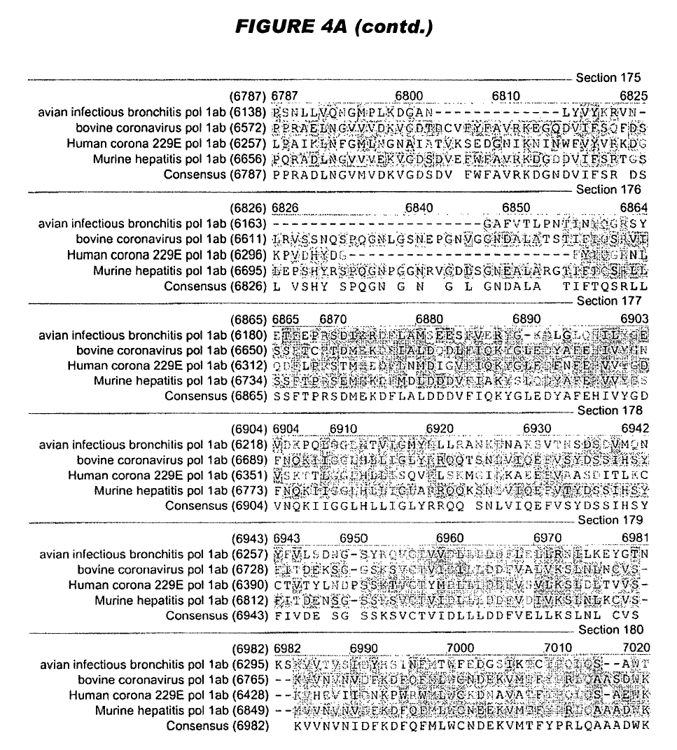

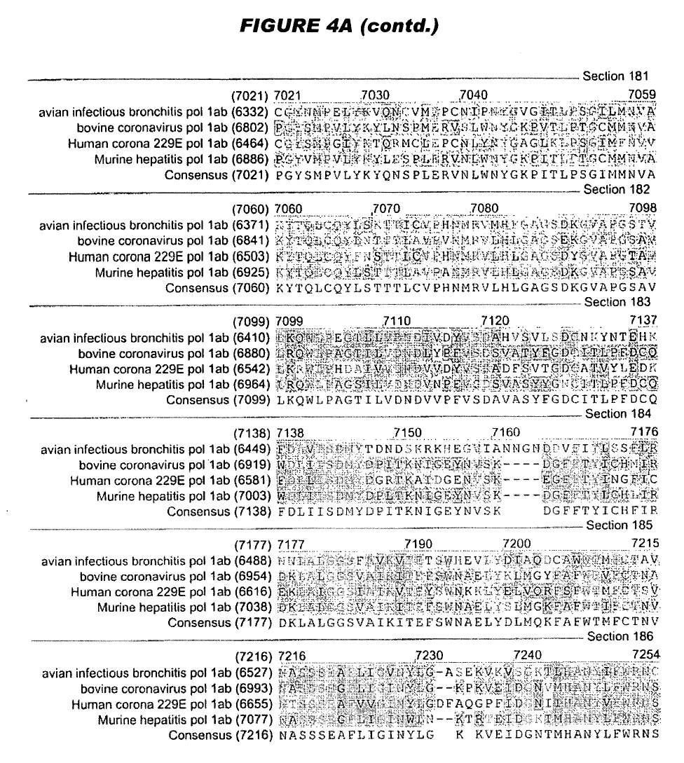

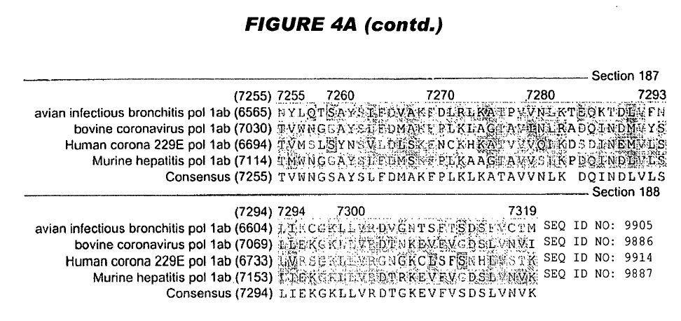

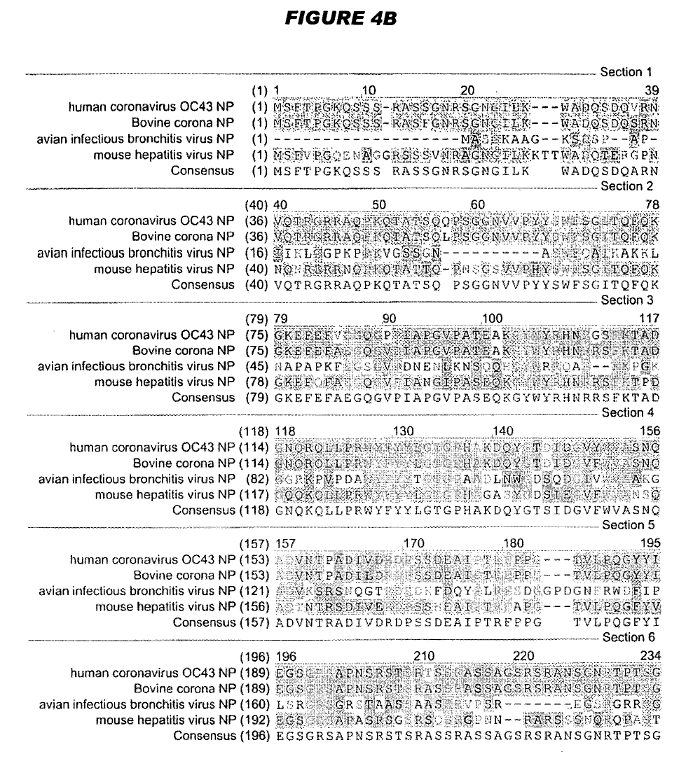

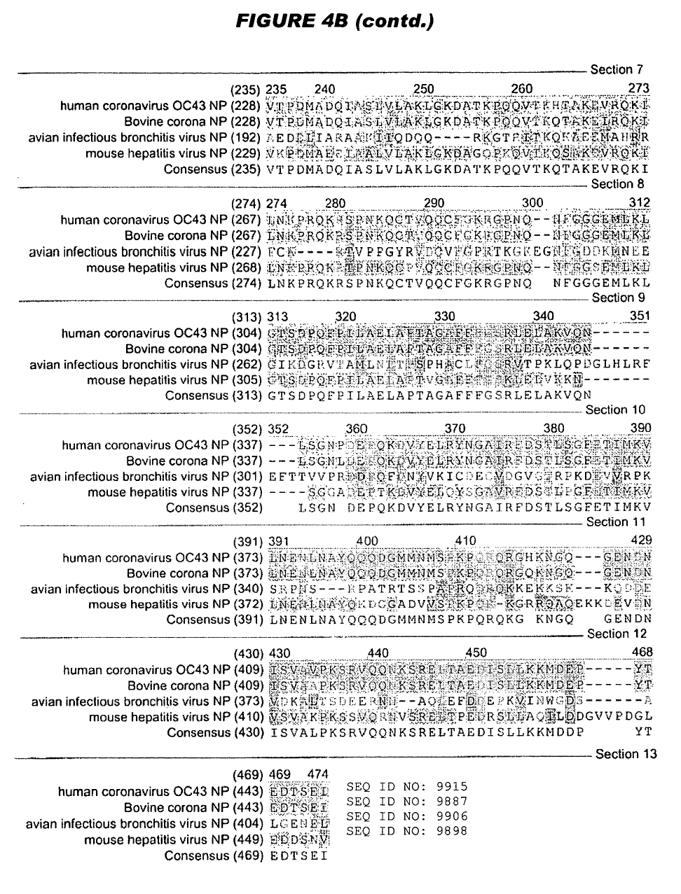

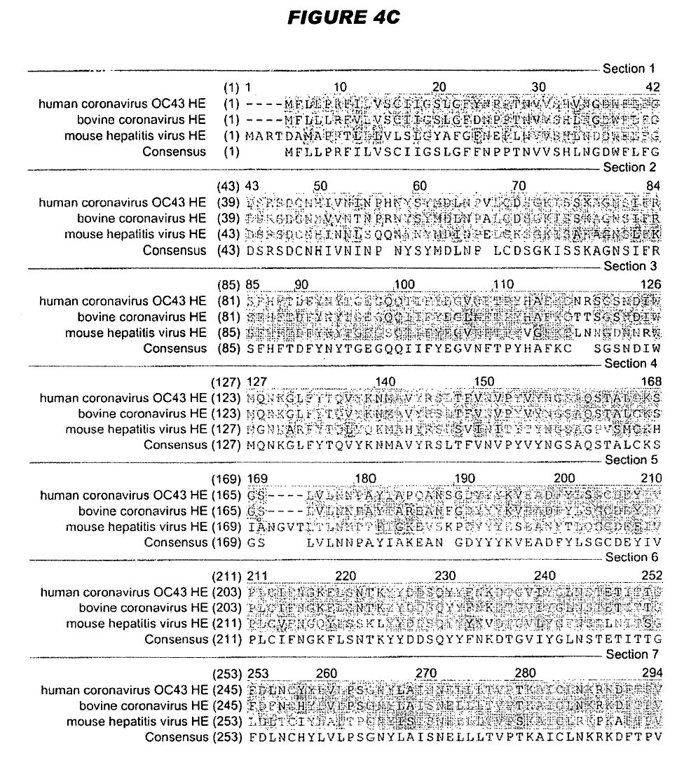

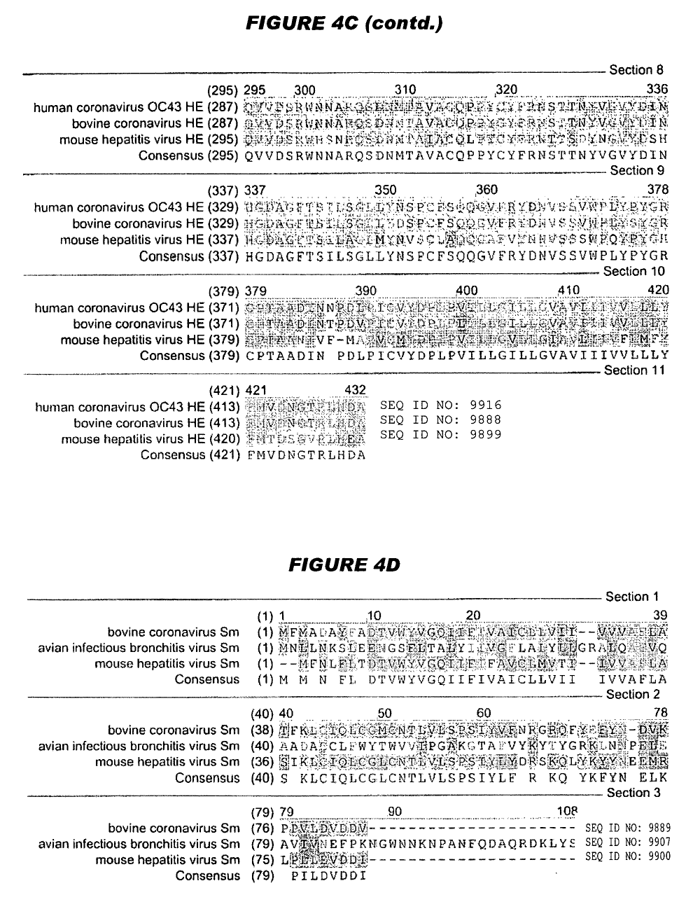

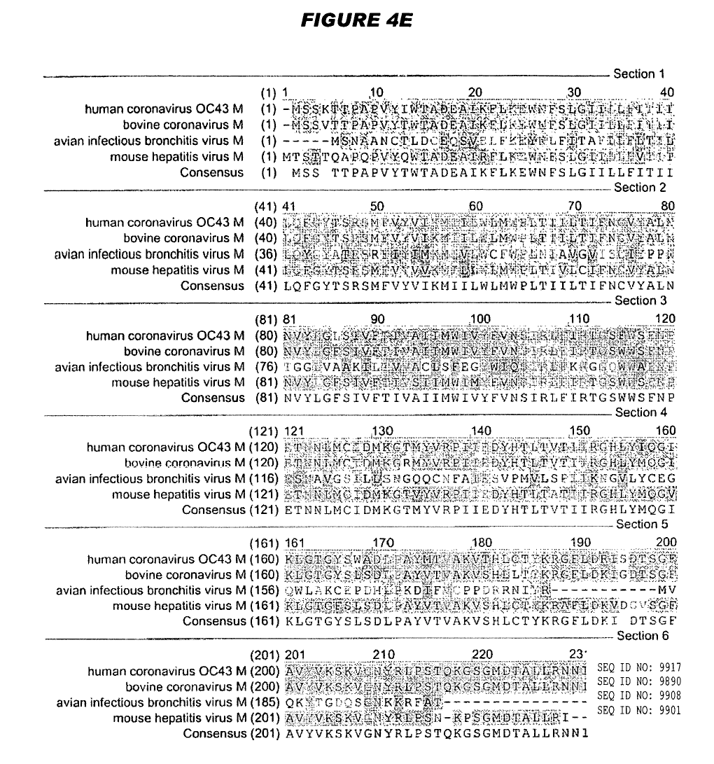

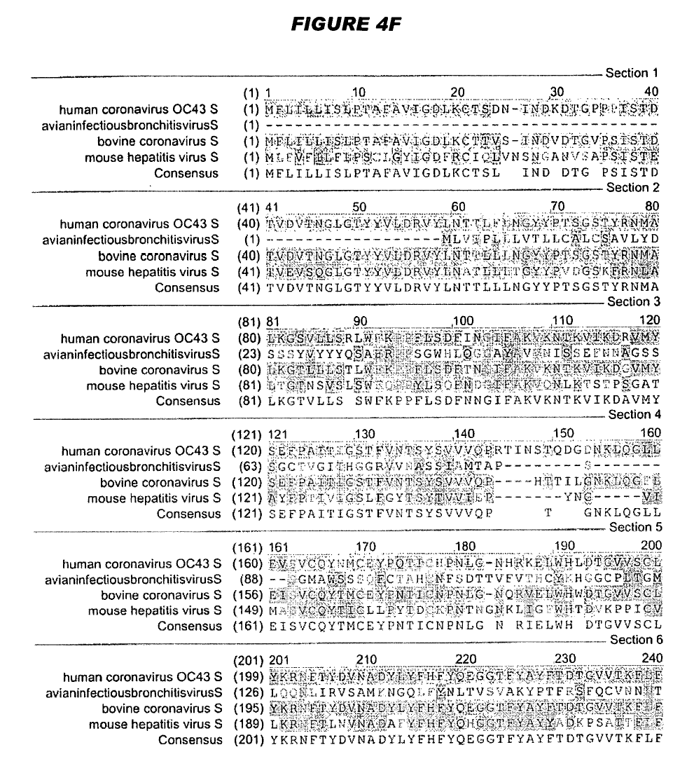

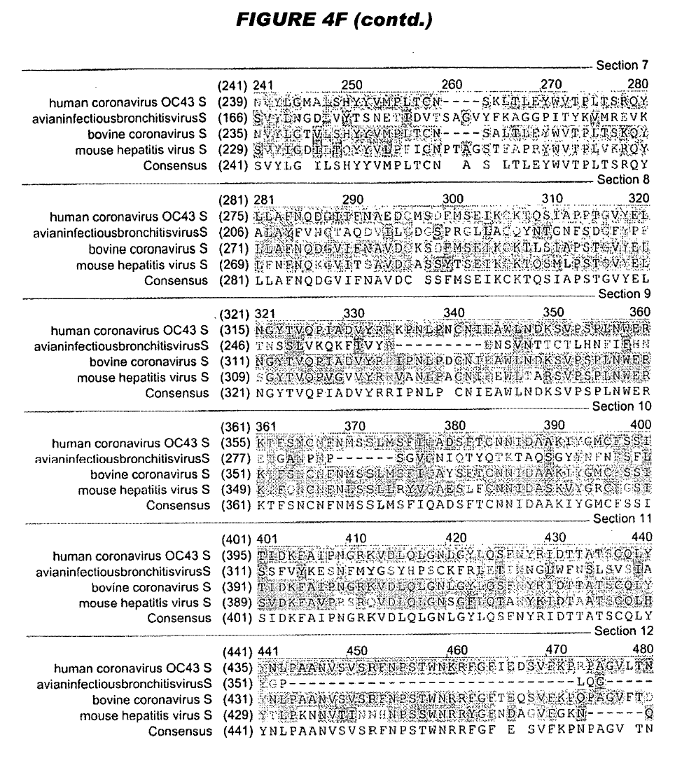

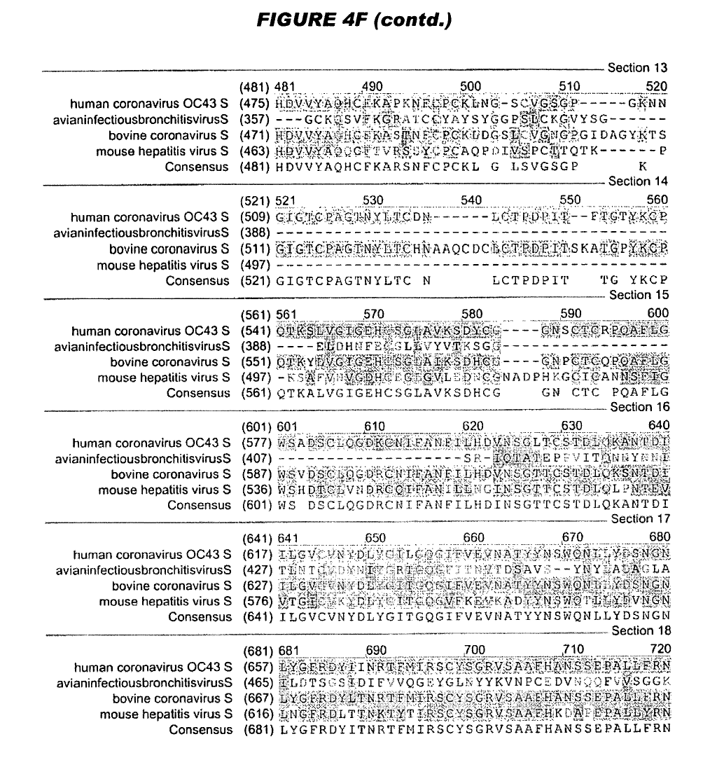

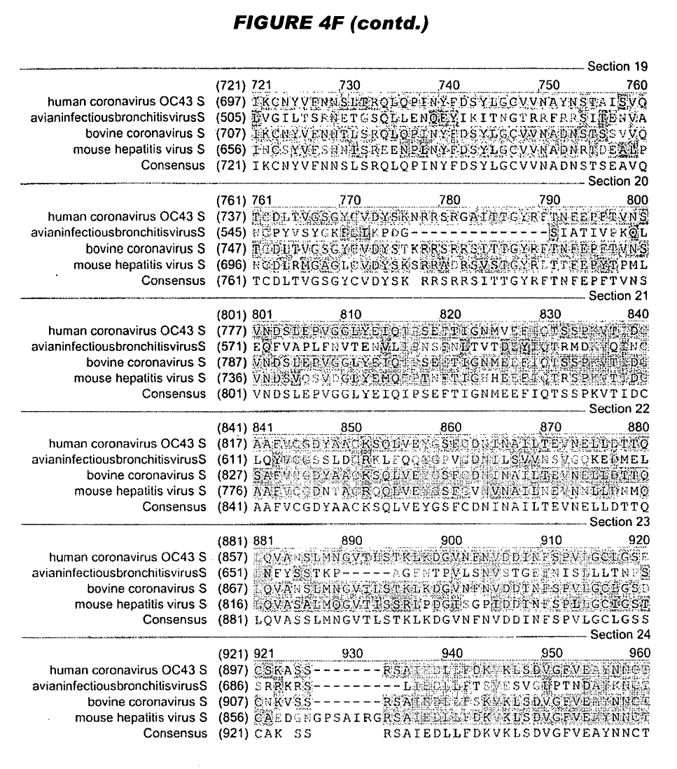

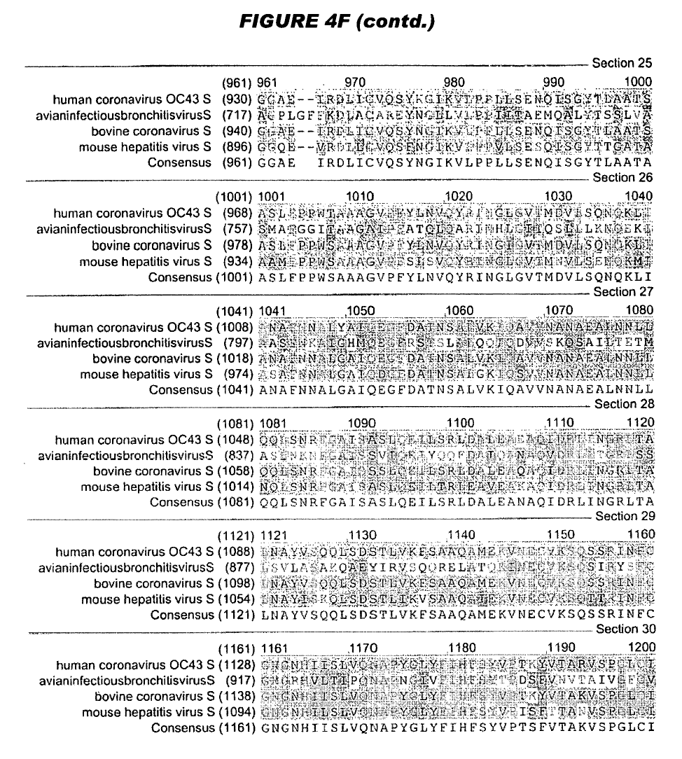

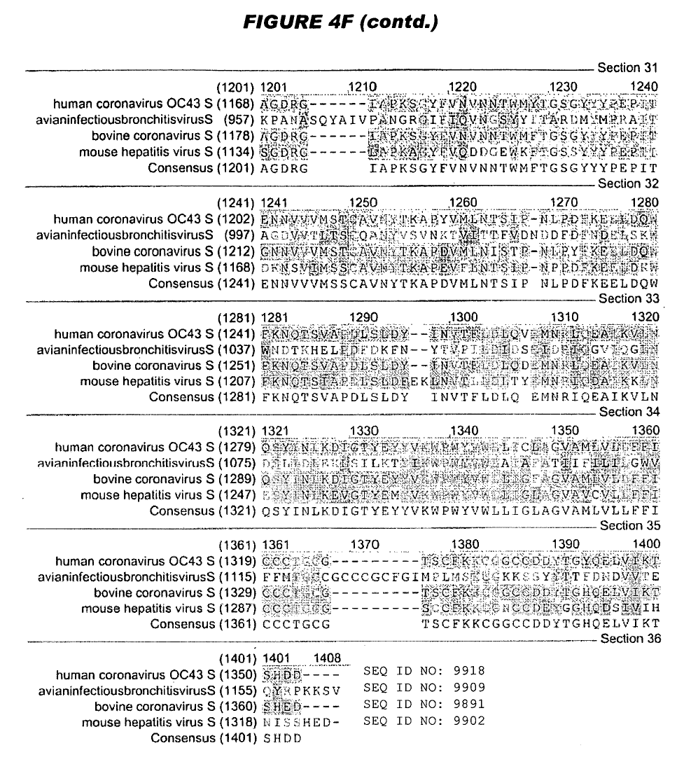

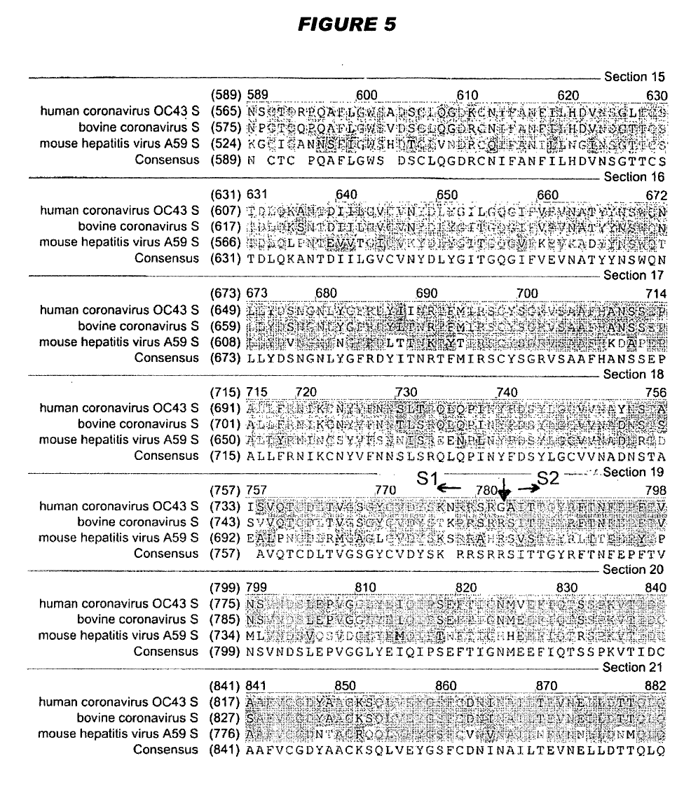

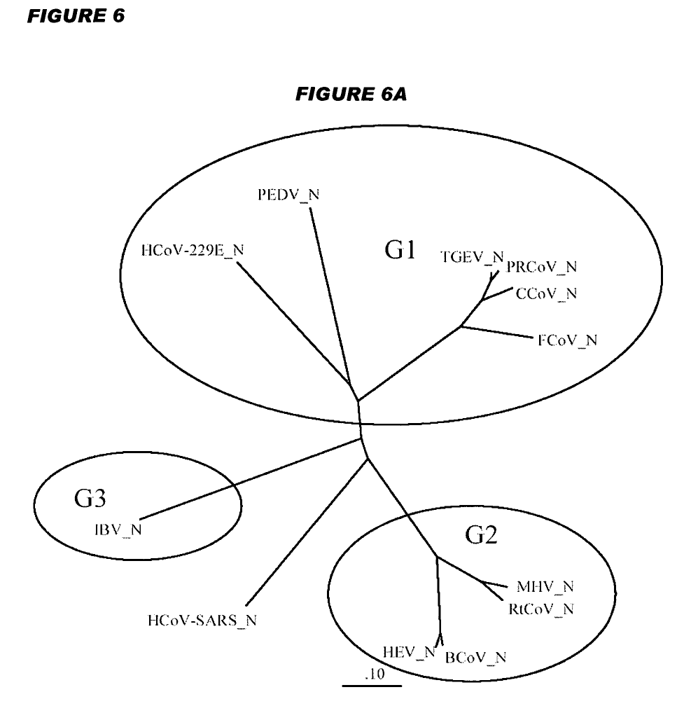

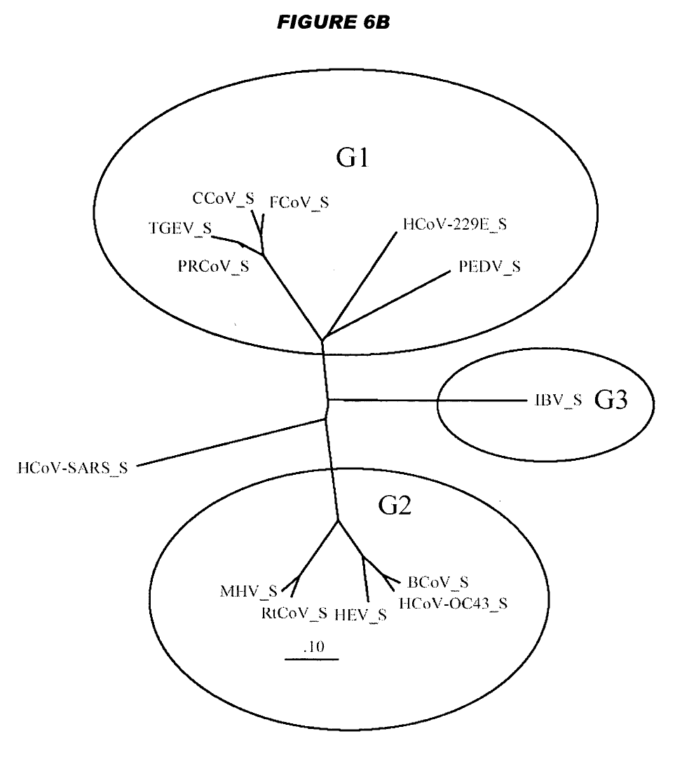

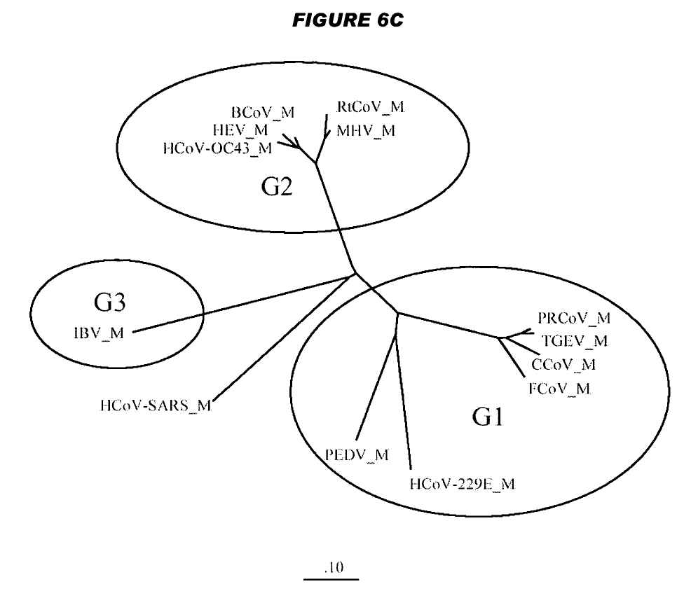

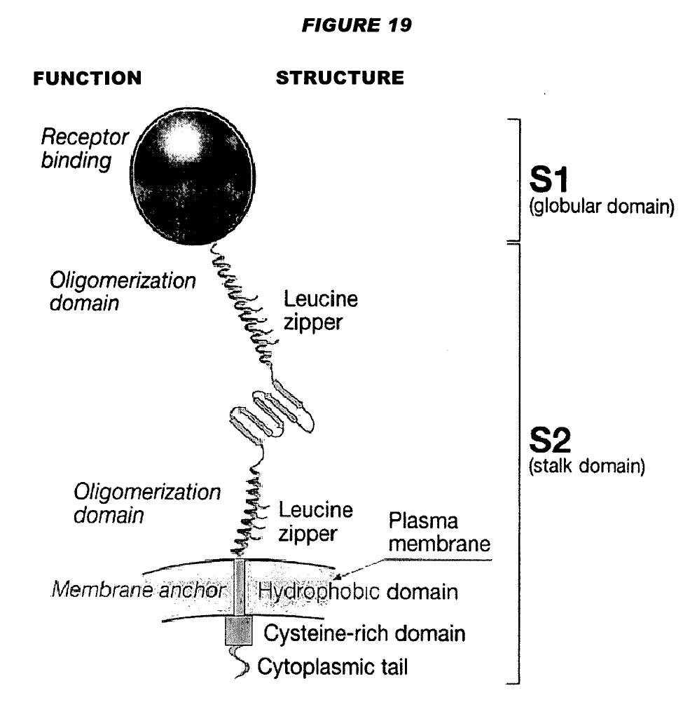

Preferably, the fusion protein comprises an amino acid sequence of a SARS virus spike protein, or a fragment thereof. Specific fragments of the spike protein which may be used in the fusion protein include the S1 domain and the S2 domain. Further fragments of the spike protein which may be used in the fusion protein include regions of each of the S1 and S2 domains, including the receptor binding region of the S1 domain, the oligomerization domain regions of the S2 domain, the leucine zipper regions of the S2 domain, the membrane anchor region of the S2 domain, the hydrophobic domain region of the S2 domain, the cystein-rich domain region of the S2 domain, and the cytoplasmic tail region of the S2 domain. (See FIG. 19). Amino acid sequences of the Spike protein corresponding to these regions can be identified by those skilled in the art, including, for example, using the functional predictions set forth earlier in the application (predicted transmembrane helices, predicted N-terminus signaling regions, predicted coiled-coil regions, etc.) as well as by homology comparison to the sequences of other known Coronaviruses (See FIGS. 4F and 5).

The fusion protein may further comprise a second amino acid sequence. Said second amino acid sequence may comprise a polypeptide sequence which facilitates protein expression or purification, preferably one of the tag sequences discussed above. Alternatively, said second amino acid sequence may comprise a second amino acid sequence from a SARS virus. Alternatively, said second amino acid sequence may comprises an amino acid sequence from another virus or bacteria, including one or more of the viruses or bacteria identified in Section I, below.

Said second amino acid sequence may comprise an amino acid sequence from another respiratory virus. Said second amino acid sequence may comprise an amino acid sequence from a virus selected from the group consisting of coronavirus, influenza virus, rhinovirus, parainfluenza virus (PIV), respiratory syncytial virus (RSV), adenovirus, and metapneumovirus.

In one embodiment, said second amino acid sequence may comprise an amino acid sequence from an adjuvant, including one or more of the adjuvants identified in section I, below.

In one embodiment, the invention includes a fusion protein comprising an amino acid sequence of a SARS virus spike protein or a fragment thereof. The fusion protein may further comprise a second amino acid sequence comprising an amino acid sequence selected from the group consisting of a second SARS virus protein, a non-SARS virus protein, a bacterial protein, and an adjuvant.

(a) Bacterial Expression of Subunit SARS Vaccines

In one embodiment, bacterial host cells are used for recombinant expression of SARS virus proteins. Bacterial host cells suitable for use in the invention include, for example, E. coli, Bacillus subtilis, and Streptococcus spp.

The SARS viral protein may be modified to facilitate bacterial recombinant expression. In particular, the SARS spike protein may be modified to facilitate transport of the spike protein to the surface of the bacterial host cell.

Applicants have discovered that there is strong structural homology between the SARS virus spike protein and the NadA protein of Neisseria meningitidis. Both proteins have an N-terminal globular “head” domain (amino acids 24-87), an intermediate alpha-helix region with high propensity to form coiled-coil structures (amino acids 88-350), and a C-terminal membrane anchor domain formed by four amphipatix transmembrane beta strands (amino acids 351-405 of NadA). In addition, a leucine zipper motive is present within the coiled-coil segment. See, FIG. 19 depicting the SARS spike protein structure Comanducci et al., “NadA, a Novel Vaccine Candidate of Neisseria meningitidis”, J. Exp. Med. 195 (11): 1445-1454 (2002). In addition, a leucine zipper motif of NadA is present within the coiled-coil segment. The NadA protein also forms high molecular weight surface-exposed oligomers (corresponding to three or four monomers) anchored to meningococcal outer membrane.

When the NadA protein is expressed in E. coli, the full-length protein is assembled in oligomers anchored to the outer membrane of E. coli, similar to the way the protein is presented in meningococcus. The NadA protein devoid of the predicted membrane anchor domain is then secreted into the culture supernatant. This secreted protein is soluble and still organized in trimers.

The invention therefore includes a fusion protein comprising an amino acid sequence of a SARS virus spike protein or a fragment thereof and a second amino acid sequence of a bacterial adhesion protein or a fragment thereof. Preferably, said adhesion protein is selected from the group consisting of NadA, YadA (of enteropathogenic Yersinia), and UspA2 (of Moraxella catarrhalis). Additional NadA-like proteins include serum resistance protein DsrA of Haemophilus ducreyi, the immunoglobulin binding proteins EibA, C, D, and F of E. coli, outer membrane protein 100 of Actinobacillus actinomycetemcomitans, the saa gene carried on the large virulence plasmid present in shiga toxigenic strains of E. coli (STEC), and each of the bacterial adhesion proteins described in U.K. Patent Application No. 0315022.4, filed on Jun. 26, 2003, each of which are specifically incorporated herein by reference.

Preferably, said adhesion protein comprises NadA or a fragment thereof.

Such fusion proteins may be used to facilitate recombinant expression of immunogenic portions of SARS surface antigens, such as spike. These fusion constructs may also allow the SARS S1 and/or S2 domains to adapt to a native confirmation. These fusion proteins are also able to oligomerize and form dimers or trimers, allowing the S1 and/or S2 domains to associate and adapt conformations as in the native SARS spike protein. Further, these expression constructs facilitate surface exposure of the SARS spike protein.

The fusion proteins of the invention preferably comprise a leader peptide from a NadA like protein, preferably NadA, a polypeptide from the immunogenic “head” region of the spike protein, and a stalk region from either the NadA like protein or the Spike protein. During expression and processing of the fusion protein, one or more amino acids may be cleaved off or removed, such as, i.e., the leader peptide or a membrane anchor domain.

The stalk regions facilitate oligomerization of the expression protein. Optionally, the fusion proteins of the invention further include an anchor region of a NadA like protein. This anchor region allows the expression fusion protein to anchor and assemble on the bacterial cell surface.

The fusion proteins of the invention include the following constructs:

(i) the NadA leader peptide (optionally also including the first 6 amino acids of the mature NadA protein to facilitate processing of the leader peptide and appropriate maturation of the protein) followed by the Spike S1 domain. Preferably, this construct comprises amino acids 1-29 of NadA (corresponding to the NadA leader peptide and the first 6 amino acids of the mature NadA protein, as shown in FIG. 22 and as set forth below) followed by amino acids 14-662 of a SARS virus Spike protein (corresponding to the S1 domain, see FIG. 19 and SEQ ID NO: 6042 and as set forth below). Specifically, construct (i) comprises SEQ ID NO: 7302.

(ii) the NadA leader peptide (optionally also including the first 6 amino acids of the mature NadA protein to facilitate processing of the leader peptide and appropriate maturation of the protein) followed by the Spike S1 domain, followed by the stalk and anchor membrane domains of NadA. Preferably, this construct comprises amino acids 1-29 of NadA (corresponding to the NadA leader peptide and the first 6 amino acids of the mature NadA protein, as shown in FIG. 22 and as set forth below) followed by amino acids 14-662 of a SARS virus Spike protein (corresponding to the S1 domain, see FIG. 19 and SEQ ID NO: 6042 and as set forth below) followed by amino acids 88-405 of NadA (corresponding to the stalk and the anchor membrane domains). Specifically, construct (ii) comprises SEQ ID NO: 7303.

(iii) the NadA leader peptide (optionally also including the first 6 amino acids of the mature NadA protein) followed by a SARS virus Spike S1 domain, followed by the NadA stalk domain. Preferably, this construct-comprises amino acids 1-29 of NadA followed by amino acids 14-662 of a SARS virus Spike protein (corresponding to the S1 domain), followed by amino acids 88-350 of NadA (corresponding to the stalk domain). Specifically, construct (iii) comprises SEQ ID NO: 7304.

(iv) the NadA leader peptide (optionally also including the first 6 amino acids of the mature NadA protein), followed by a SARS virus Spike S1 and S2 domain (excluding the putative transmembrane region), followed by the anchor domain of NadA. Preferably, this construct comprises amino acids 1-29 of NadA, followed by amino acids 14-1195 of a SARS virus Spike protein (corresponding to S1 and S2, excluding the putative transmembrane region), followed by amino acids 351-405 of NadA (corresponding to the NadA anchor domain). Specifically, construct (iv) comprises SEQ ID NO: 7305. Alternatively, the NadA anchor domain may comprise amino acids 332-405 of NadA.

(v) the NadA leader peptide (optionally also including the first 6 amino acids of the mature NadA protein), followed by a SARS virus Spike S1 and S2 domain (exclusing the putative transmembrane region). Preferably, this construct comprises amino acids 1-29 of NadA, followed by amino acids 14-1195 of a SARS virus Spike protein. Specifically, construct (v) comprises SEQ ID NO: 7306.

In each of constructs (i) to (v), the first 23 amino acids are the NadA leader peptide, and the GS dipeptide at residues 679-680 arises from the insertion of a restriction enzyme site.

In constructs (i), (ii) and (iii), the NadA “head” is replaced by the Spike S1 domain, and the fusion proteins are anchored to the outer membrane of E. coli or secreted in the culture supernatant, respectively. In constructs (iv) and (v), the “head” and “stalk” domains of NadA are replaced by S1 and S2 Spike domains; also in this case, the two fusion proteins are anchored to the outer membrane of E. Coli or secreted in the culture supernatant, respectively.

Accordingly, the invention further includes a fusion protein comprising an amino acid sequence of a SARS virus spike protein or a fragment thereof and a second amino acid sequence of a bacterial adhesion protein or a fragment thereof. Preferably, amino acids corresponding to the “head” of the adhesion protein are replaced by amino acids corresponding to a SARS virus Spike S1 domain. Alternatively, the amino acids corresponding to the “head” and “stalk” domains of the bacterial adhesion protein are replaced by amino acids corresponding to the SARS virus spike protein S1 and S2 domains.

As discussed above and shown in FIG. 19, the S1 domain of the Spike protein is identified as the globular receptor binding “head” region. The S1 domain of the Spike protein preferably comprises about amino acids 14-662 of SEQ ID NO: 6042. The S1 domain may comprise a shorter amino acid sequence, wherein amino acids are removed from either the N-terminal or C-terminal regions. Preferably, 3, 5, 7, 9, 13, 15, 20 or 25 amino acids are removed from either the N-terminal or C-terminal regions. The S1 domain further includes amino acid sequences having sequence identity to the S1 region of SEQ ID NO: 6042. An example of the S1 domain is SEQ ID NO: 7307:

As discussed above and shown in FIG. 19, the S2 domain of the Spike protein is identified as the “stalk” region. The “stalk” region comprises oligomerization domain regions, a leucine zipper domain regions, membrane anchor regions, hydrophobic domain regions, cystein-rich domain region and a cytoplasmic tail region. The S2 domain of the Spike protein preferably excludes the transmembrane region and comprises about amino acids 663-1195 of SEQ ID NO: 6042. The S2 domain may comprise a shorter amino acid sequence, wherein amino acids are removed from either the N-terminal or C-terminal regions. Preferably, 3, 5, 7, 9, 13, 15, 20 or 25 amino acids are removed from either the N-terminal or C-terminal regions. The S2 domain further includes amino acid sequences having sequence identity to the S2 region of SEQ ID NO: 6042. An example of the S1 domain (with the transmembrane region excluded) is SEQ ID NO: 7308.

An example of the NadA protein described above is SEQ ID NO: 7309. As discussed above, the leader sequence of NadA used in the fusion protein preferably comprises about the first 29 amino acids of NadA (including a leader sequence with about 6 amino acids of the NadA head protein). Examples of such a leader sequences are set forth as SEQ ID NOS: 7310 and 7311 below. The fusion protein may use a leader sequence comprising a shorter amino acid sequence, wherein amino acids are removed from either the N-terminal or C-terminal regions. Preferably, 1, 2, 3, 4, or 5 amino acids are removed from either the N-terminal or C-terminal end of the sequence. The leader sequence used in the fusion protein may also include an amino acid sequences having sequence identity to SEQ ID NO: 7310 or SEQ ID NO: 7311. Preferably, the leader sequence comprises SEQ ID NO: 7311.

Optionally, the fusion peptide comprises about the first 6 amino acids of the mature NadA protein to facilitate processing of the leader peptide and appropriate maturation of the protein. An examples of the first 6 amino acids of a mature NadA proteins is SEQ ID NO: 7312.

As discussed above, the stalk and anchor sequences of NadA used in the fusion protein preferably comprise about amino acids 88-405 of NadA. An example of an amino acid sequence comprising NadA stalk and anchor regions is set forth below as SEQ ID NO: 7313 below. An example of an amino acid sequence comprising a NadA stalk region (without the anchor region) is set forth as SEQ ID NO: 7314 below. An example of an amino acid sequence comprising a NadA anchor region is set forth as SEQ ID NO: 7315 below. The fusion protein may use a stalk (and/or anchor) sequence comprising a shorter amino acid sequence, wherein amino acids are removed from either the N-terminal or C-terminal regions. Preferably, 1, 2, 3, 4, 5, 6, 7, 8 or 9 amino acids are removed from either the N-terminal or C-terminal end of the sequence. The leader sequence used in the fusion protein may also include an amino acid sequences having sequence identity to the SEQ ID NO: 7313.

The fusion proteins of the invention, including those described above, may be prepared, for example, as follows. Single fragments (such as the regions described above) may be amplified by PCR using the oligonucleotide primers set forth in the Table below. (S1_{L} refers to the Spike protein fused to the leader peptide of NadA; S2 refers to the stalk region of the Spike protein, with and without the stop codon). The oligonucleotides were designed on the basis of the DNA sequence of NadA from N. meningitidis B 2996 strain and of Spike from SARS virus isolate FRA1. Each oligonucleotide includes a restriction site as a tail in order to direct the cloning into the expression vector pET21b.

| | | SEQ ID NO: | Restriction site |
|---|---|---|---|
| S1_{L} | For | 7316 | NdeI |
| S1_{L} | Rev | 7317 | BamHI |
| S2 | For | 7318 | BamHI |
| S2 | Rev | 7319 | HindIII |
| S2-stop | Rev | 7320 | XhoI |
| NadA₈₈ | For | 7321 | BamHI |
| NadA₃₅₀ | Rev | 7322 | XhoI |
| NadA₃₃₂ | For | 7323 | HindIII |
| NadA₄₀₅ | Rev | 7324 | XhoI |

The single fragments are sequentially cloned into pET21b vector, in order to express the proteins under the control of inducible T7 promoter. The S1 domain of the Spike protein fused to the leader peptide of NadA (S1_{L}) was obtained by PCR using the primers S1_{L}-For and S1_{L}-Rev. The forward oligonucleotide primer contains the NdeI restriction sequence and the sequence coding for the leader peptide of NadA plus the first 6 aminoacids of the mature protein. The PCR fragment was cloned as a NdeI/BamHI fragment in the pET21b vector opened with the same restriction enzymes. This clone (PET-S1_{L}) was then used to sequentially clone the other different domains, as BamHI/XhoI, BamHI/HindIII or HindIII/XhoI fragments. BamHI and HindIII restriction sites introduce the aminoacids GS and KL, respectively.

The PCR amplification protocol was as follows: 200 ng of genomic DNA from Neisseria meningitidis 2996 or 10 ng of plasmid DNA preparation (plasmid pCMVnew, containing the entire gene coding of the Spike protein), were used as template in the presence of 40 μM of each oligonucletide primer, 400-800 μM dNTPs solution, 1× PCR buffer (including 1.5 mM MgCl₂), 2.5 units TaqI DNA polymerase (using Perkin-Elmer AmpliTaQ or Invitrogen Platinum Pfx DNA polymerase).

After a preliminary 3 minute incubation of the whole mix at 95° C., each sample underwent a two-step amplification: the first 5 cycles were performed using the hybridisation temperature that excluded the restriction enzyme tail of the primer (Tm1). This was followed by 30 cycles according to the hybridisation temperature calculated for the whole length oligos (Tm2). Elongation times, performed at 68° C. or 72° C., varied according to the length of the fragment to be amplified. The cycles were completed with a 10 minute extension step at 68° C. or 72° C.

The amplified DNA was either loaded directly on agarose gel and the DNA fragment corresponding to the band of correct size was purified from the gel using the Qiagen™ Gel Extraction Kit, following the manufacturer's protocol.

The purified DNA corresponding to the amplified fragment and the plasmid vectors were digested with the appropriate restriction enzymes, purified using the QIAquick™ PCR purification kit (following the manufacturer's instructions) and ligation reactions were performed.

The ligation products were transformed into competent E. coli DH5α and screening for recombinant clones was performed by growing randomly-selected colonies and extracting the plasmid DNA using the Qiagen QIAprep Spin Miniprep Kit, following the manufacturer's instructions.

Recombinant plasmids were introduced into E. coli BL21(DE3) used as expression host. Single recombinant colonies were inoculated into LB+ ampicillin and incubated at 37° C. for 14-16 h. Bacteria were directly recovered by centrifugation (uninduced conditions) or diluted in fresh medium and grown at 37° C. until OD₆₀₀ between 0.4-0.8. Protein expression was induced by addition of 1 mM Isopropyl-1-thio-β-D-galactopyranoside (IPTG) for three hours (induced conditions).

Whole cell lysates were obtained resuspending bacteria in SDS-sample buffer 1× and boiling for 5-10 min. Equal amounts of proteins were separated using NuPAGE (Invitrogen) or BIORAD Gel System, according to the manufacturer's instructions. Proteins were revealed by Coomassie-blue staining or transferred onto nitrocellulose membranes for western blot analysis. Western blot was performed using a rabbit polyclonal anti-serum against purified NadA_{Δ351-405} (diluted 1:3000) and a secondary peroxidase-conjugate antibody (DAKO).

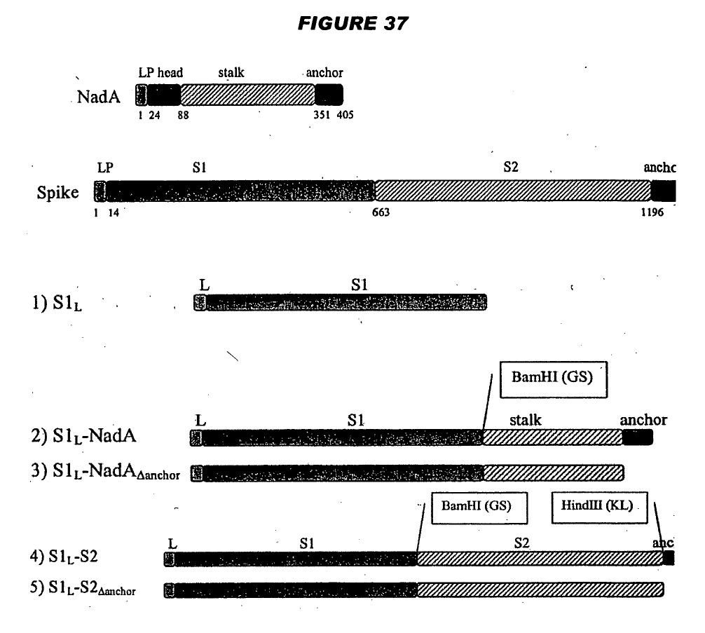

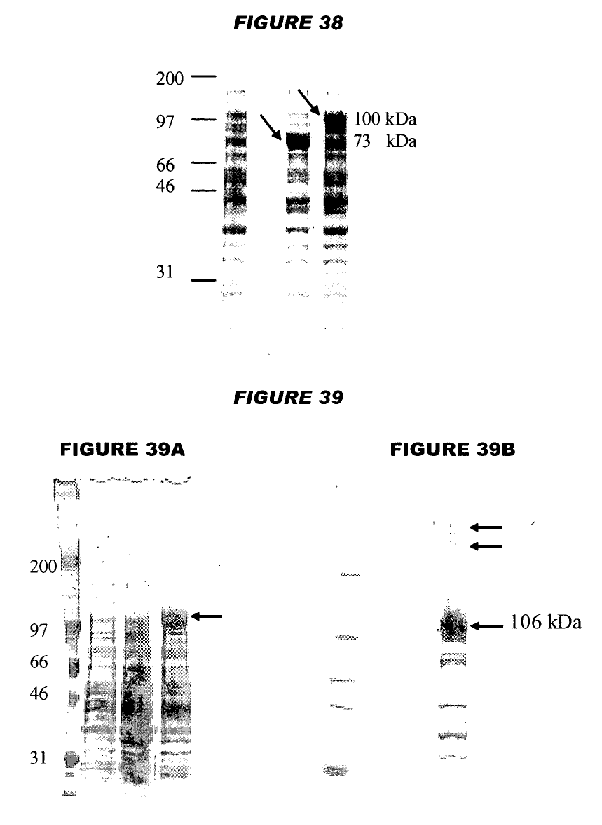

Results of the expression in E. coli of S1_{L}, S1_{L}-NadA and S1_{L}-Nad_{Δanchor} are shown in FIGS. 38 and 39. Schematics of the fusion constructs are shown in FIG. 37.

Bacterial expression of the SARS viral antigens may also be used to prepare compositions comprising outer membrane vesicles wherein said outer membrane vesicles comprise one or more SARS viral antigens.

Outer Membrane Vesicles (“OMV”), also referred to as blebs, refer to vesicles formed or derived from fragments of the outer membrane of a Gram negative bacterium. OMVs typically comprise outer membrane proteins (OMPs), lipids, phospholipids, periplasmic material and lipopolysaccharide (LPS). Gram negative bacteria often shed OMVs during virulent infections in a process known as blebbing. OMVs can also be obtained from Gram negative bacteria via a number of chemical denaturation processes, such as detergent extraction. Synthetic OMVs or liposomes, comprising a lipid bilayer and typically enclosing an aqueous core, can also be prepared with the SARS viral antigens of the invention.

The OMVs of the invention are preferably lipid vesicles comprising a lipid bilayer surrounding an aquous core. Typically the lipid vesicles are of unilamellar structure (i.e., a single lipid bilayer surrounds the aquous core), although multilammelar lipid vesicles may also be used in the compositions of the invention. OMVs typically have sizes in the nanomolar to micromolar range, e.g., from 1 nM to 100 μM, more typically from 10 nM to 10 μM and preferably from 30 nM to 1 μM.

The OMVs of the invention are preferably prepared from gram negative bacteria. Gram negative bacteria are those bacteria that fail to resist decolorization in the commonly known Gram staining method. Gram negative bacteria are characterized by a complex multilater cell wall and often possess an outer layer polysaccharide capsule. Gram negative bacteria suitable for producing OMVs include, for example, species from Neisseria, Moraxella, Kingella, Acinetobacter, Brucella, Bordetella, Chlamydia, Porphyromonas, Actinobacillus, Borelia, Serratia, Campylobacter, Helicobacter, Haemophilus, Escherichia, Legionella, Salmonella, Pseudomonas and Yersinia.

The OMVs of the invention preferably comprise one or more SARS viral antigens or a fragment thereof. The SARS viral antigens may be recombinantly expressed in a Gram negative bacterial host cell and then harvested with the OMV.

Antigenic components, such as recombinantly expressed SARS viral antigens, may be located in any or all of the three main compartments of the lipid vesicles, including attached to either the interior or exterior surface of the lipid vesicle, for example via a membrane anchor domain, or attachment to a lipid moiety; inserted into the lipid bilayer, for example where the antigenic component is itself a hydrophobic or lipid based entity; or located within the aqueous center or core of the lipid vesicle.

Synthetically prepared OMVs, or liposomes, may be used in the invention. Such liposomes may comprise a number of different lipids and fatty acids. Suitable lipids for inclusion in liposomes of the invention include but are not limited to phophatidylinositol-(4,5)-diphosphate, phosphatidylserine, phosphatidylcholine, phosphatidylethanolamine, phosphatidyglycerol, cholesterol, beta-oleolyl-gamma-palmitoyl, lipopolysaccharides and galactocerbrosides.

Suitable means for extraction of OMVs from bacterial sources include deoxycholate extraction, Tris/HCl/EDTA extraction, and lithium acetate extraction. Preferably, the extraction process comprises a physical and/or chemical means to disrupt the bacterial cell outer membrane in order to release sufficient OMVs for purification and isolation. See, e.g., WO 03/051379.

The OMVs of the invention may be enriched and/or supplemented with antigenic components, such as SARS viral antigens, by methods known in the art, including, for example, direct combination in vitro where an energetic combination step can optionally be applied to facilitate integration of the antigenic component into a compartment of the liposome. Methods of energetic combination suitable for use in the invention include homogenization, ultrasonication, extrusion, and combinations thereof.

Preferably, the antigenic component, such as the SARS viral antigen, is recombinantly produced by the host cell from which the OMV is derived. In one embodiment, such OMVs are prepared by introducing nucleic acid sequence encoding for the SARS viral antigen into the recombinant host cell. Preferably the nucleic acid sequence encoding for the SARS viral antigen is controlled by a strong promoter sequence. Preferably, the nucleic acid sequence encoding the SARS viral antigen further comprises an outer-membrane targeting signal. For example, the nucleic acid sequence encoding the SARS viral antigen may be fused to a sequence encoding for a naturally occurring outer membrane protein of the bacterial host. Preferably, the nucleic acid sequence encoding the SARS viral antigen is fused to the signal peptide sequence of the naturally occurring outer membrane protein of the bacterial host.

Methods of preparing an optimizing OMVs for use in vaccines are disclosed in, for example Filip et al., J. Bact. (1973) 115: 717-722; Davies et al., J. Immunol. Method (1990) 143:215-225; and WO 01/09350.

In one embodiment, a bacterial host cell, such as E. coli, are transformed to express the SARS spike protein. As discussed above, the spike protein may be modified to facilitate bacterial expression and transport of the spike protein to the surface of the host cell. Each of the Spike/NadA fusion constructs discussed above may be used in the OMV preparations of the invention. Preferably, constructs comprising the spike S1 globular head domain fused to the stalk region of NadA are used to generate OMVs. The construct may optionally include the NadA leader peptide as well as the NadA anchor peptide. Schematic diagrams of these preferred OMV constructs are depicted in FIG. 49.

Example 6 describes one method of preparing the OMVs of the invention.

(b) Mammalian Expression of Subunit SARS Vaccine

As discussed above, mammalian host cells may be used for recombinant expression of SARS virus proteins. Mammalian host cells suitable for use in the invention include, for example, Chinese hamster ovary (CHO) cells, HeLa cells, baby hamster kidney (BHK) cells, monkey kidney cells (e.g., Hep G2), Madin-Darby bovine kidney (“MDBK”) cells, as well as others. Mammalian sources of cells include, but are not limited to, human or non-human primate (e.g., MRC-5 (ATCC CCL-171), WI-38 (ATCC CCL-75), human embryonic kidney cells (293 cells, typically transformed by sheared adenovirus type 5 DNA), VERO cells from monkey kidneys (including, for example COS7 cells), horse, cow (e.g., MDBK cells), sheep, dog (e.g., MDCK cells from dog kidneys, ATCC CCL34 MDCK (NBL2) or MDCK 33016, deposit number DSM ACC 2219 as described in WO 97/37001), cat, and rodent (e.g., hamster cells such as BHK21-F, HKCC cells, or Chinese hamster ovary cells (CHO cells)), and may be obtained from a wide variety of developmental stages, including for example, adult, neonatal, fetal, and embryo.

The polynucleotides encoding the SARS viral proteins may be modified to facilitate or enhance expression. For example, commercial leader sequences known in the art, such as tPA or IgK or interleukin-2, may be used in the recombinant constructs. Preferably, however, the natural SARS leader sequence is used. Use of the natural leader sequence can be used to ensure that the protein will be trafficked in human cells in the same way as during a normal viral infection, which may be advantageous e.g. for DNA vaccines, where antigen is expressed in situ.

As discussed above, tag sequences can be used in the expression constructs to facilitate purification, detection and stability of the expressed protein. Tag proteins suitable for use in the invention include a polyarginine tag (Arg-tag), polyhistidine tag (His-tag), FLAG-tag, Strep-tag, c-myc-tag, S-tag, calmodulin-binding peptide, cellulose-binding domain, SBP-tag, chitin-binding domain, glutathione S-transferase-tag (GST), maltose-binding protein, transcription termination anti-terminiantion factor (NusA), E. coli thioredoxin (TrxA) and protein disulfide isomerase I (DsbA). Preferred tag proteins include His-tag and GST. A full discussion on the use of tag proteins can be found at Terpe et al., “Overview of tag protein fusions: from molecular and biochemical fundamentals to commercial systems”, Appl Microbiol Biotechnol (2003) 60:523-533.

After purification, the tag proteins may optionally be removed from the expressed fusion protein, i.e., by specifically tailored enzymatic treatments known in the art. Commonly used proteases include enterokinase, tobacco etch virus (TEV), thrombin, and factor Xₐ.

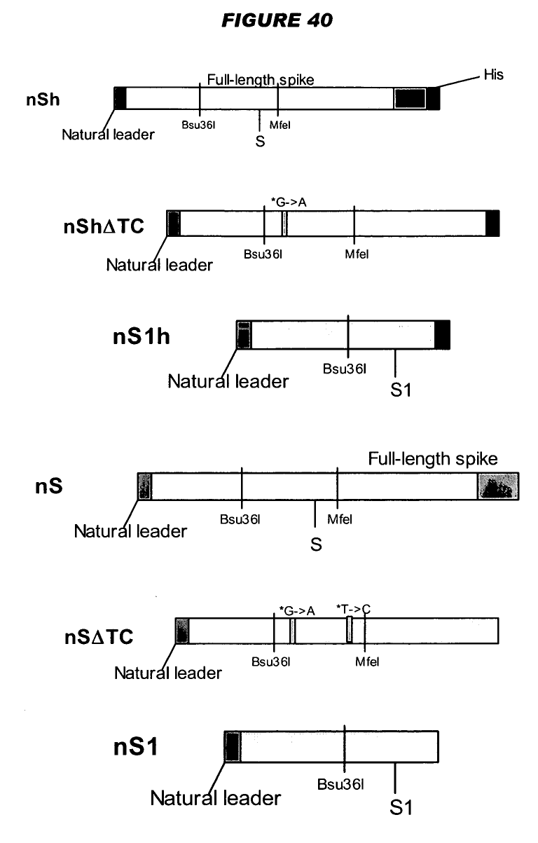

One or more amino acid sequences or amino acid domains of the spike protein may be removed to facilitate mammalian recombinant expression. For instance, the entire S2 domain or the spike transmembrane region may be removed. Representative examples of some expression constructs of both full length and truncated spike glycoprotein suitable for mammalian expression are shown in FIG. 40. Polynucleotide sequences representing each construct are shown in SEQ ID NOS 6578-6583. A description of each annotation is shown below:

| Clone | | Expression |
|---|---|---|
| Name | Description | Construct |
| nSh | natural leader sequence | SEQ ID NO: 6578 |
| | full length Spike | |
| | histidine tag | |
| nS | natural leader sequence | SEQ ID NO: 6579 |
| | full length Spike | |
| nShΔTC | natural leader sequence | SEQ ID NO: 6580 |
| | Spike without transmembrane sequence | |
| | histidine tag | |
| nSΔTC | natural leader sequence | SEQ ID NO: 6581 |
| | Spike without transmembrane sequence | |
| nS1h | natural leader sequence | SEQ ID NO: 6582 |
| | S1 domain | |
| | histidine tag | |
| nS1 | natural leader sequence | SEQ ID NO: 6583 |
| | S1 domain | |

Cloned cDNA fragments that encompass full-length Spike coding sequences, as well as a Spike construct deleted of the transmembrane and cytoplasmic domains (TM-Cy-deleted Spike) for secretion were inserted into an expression vector pCMVIII to create nSh and nShΔTC, respectively. Both spike proteins were tagged with six histidine residues at the end of C-terminus to aid initial characterization of the expressed spike proteins. Similar sequences encoding full-length Spike or transmembrane and cytoplasmic domain deleted Spike, but without the histidine “tag” are readily substituted by one of skill in the art.

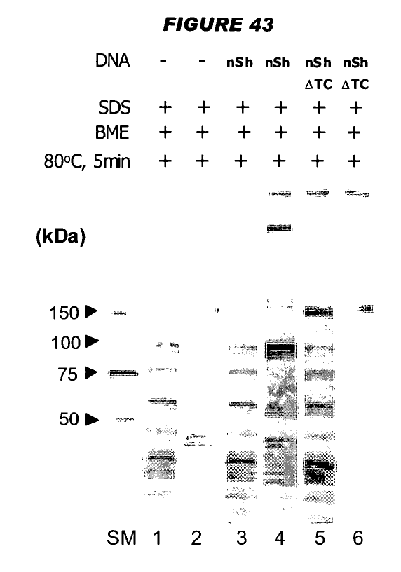

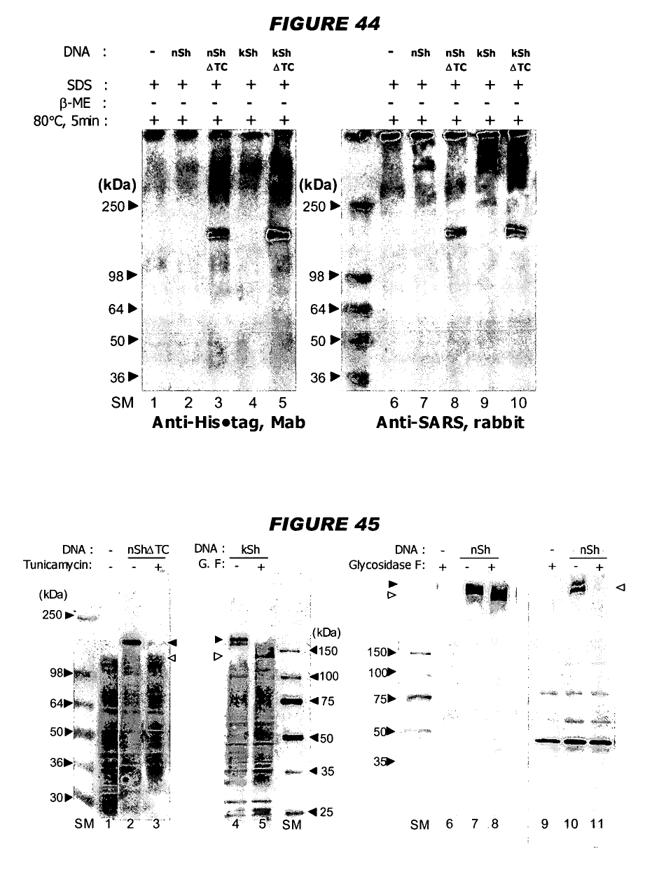

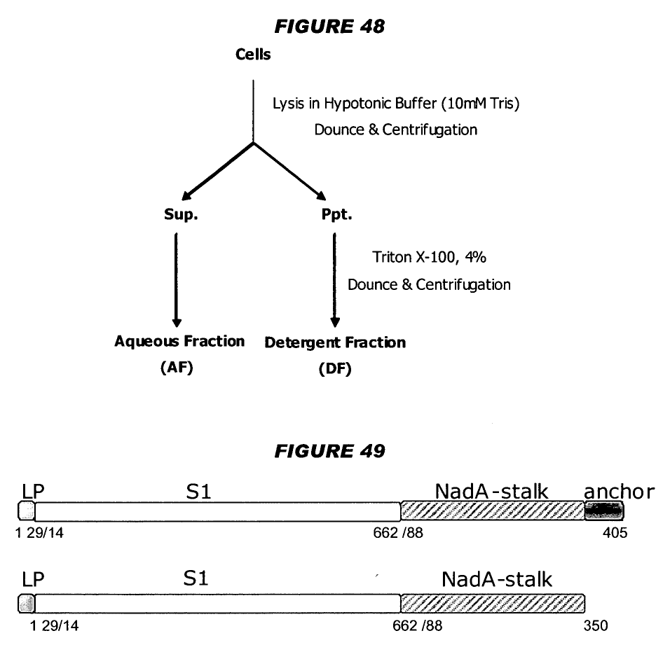

The likely locations of the expressed spike constructs was assessed by separating expressed proteins into an aqueous fraction (AF) and a detergent fraction (DF) using the procedure shown in FIG. 48, with results of western blot analysis shown in FIG. 43. The above described vector constructs were evaluated for expression after transfection into COS7 cells. The construct expressing the full length spike protein remained in the cell membrane while the construct expressing the truncated spike protein was located either in the cytosol (FIG. 43) or secreted into the cell medium (FIG. 44). As shown in FIG. 43, full-length spike protein is found in DF (membrane) in an aggregated form, while the truncated protein is found in AF (cytosol) as a monomer. As shown in FIG. 44, deleted proteins (ShΔTC) are secreted, and a small fraction of full-length spike protein is detected in the medium by rabbit serum.

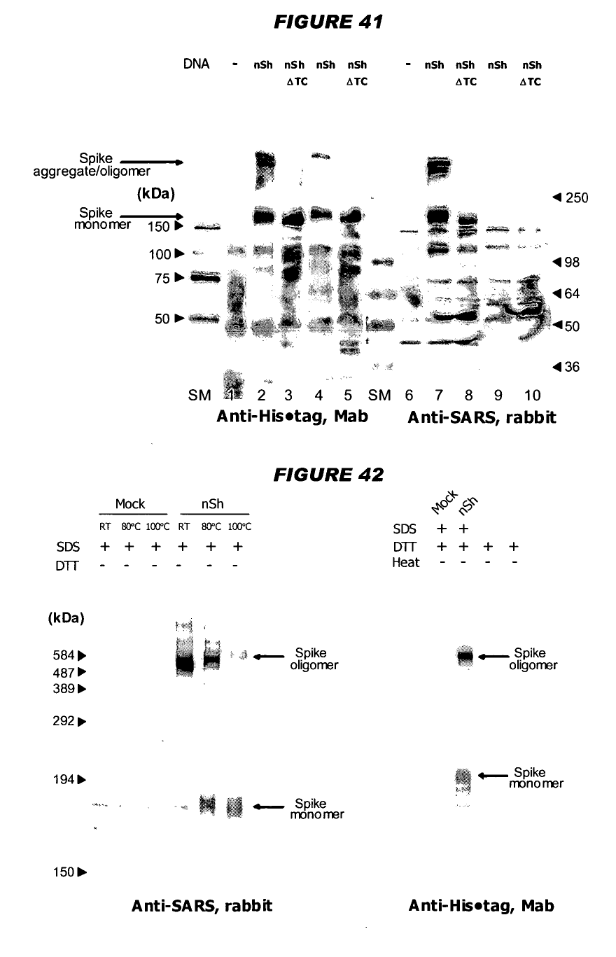

Recombinantly expressed spike proteins may be oligomerized. When the spike proteins are to be used in a vaccine or to generate antibodies specific to the spike protein, they are preferably oligomerized. In order to obtain oligomerized spike protein, it is preferred to maintain the transmembrane domain in the recombinant expression construct. For example, FIG. 41 illustrates a western blot of COS7 cell lysates comparing expressed nSh and nShΔTC using both anti-his tag and rabbit anti-SARS antibodies. As shown full-length (nSh) aggregates, but the truncated (nShΔTC) spike protein does not. Antibody raised against the His-tagged protein recognizes full-length and truncated spike proteins in native and reduced forms. Rabbit antiserum recognizes spike protein only in non-reducing conditions. Spike aggregates or oligomers were present in larger amounts in the cell lysates from the expressed nSh constructs. Preferably, the oligomerized spike proteins form a homotrimer, as indicated in FIG. 47

A further experiment, illustrated in FIG. 42, demonstrates that the oligomerization of the expressed nSh constructs is likely due to a non-covalent linkage (and is likely not due to, for example, a disulfide bond). The oligomer dissociates into monomers at elevated temperature (80-100° C.), but is stable in reducing conditions if not heated.

It is further preferred that recombinantly expressed spike proteins are glycoslyated. Tunicamycin and glycosidases were used to assess glycosylation. FIG. 45 illustrates that glycoslation of expressed spike proteins is not affected by removal of the transmembrane domain region. Both full-length (Sh) and truncated (ShΔTC) SARS spike proteins are glycosylated.

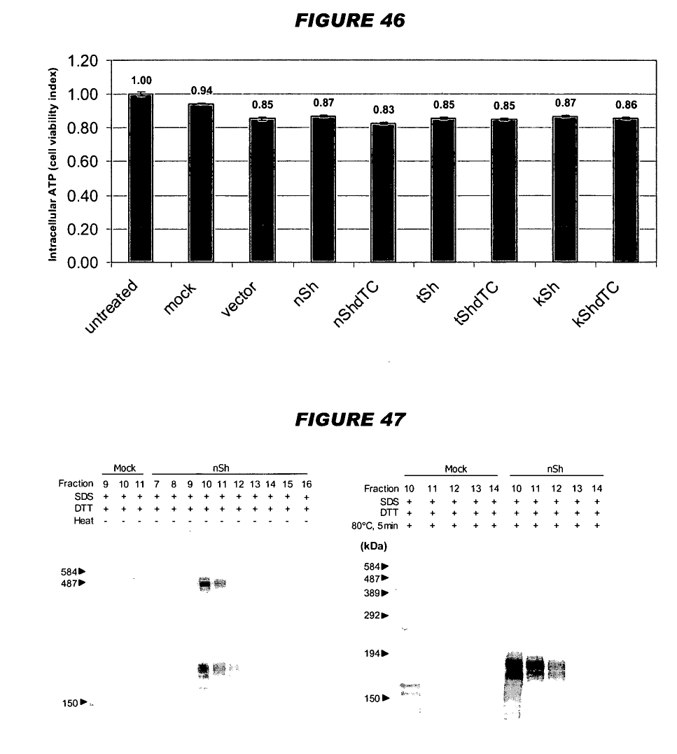

Preferably, expression of the constructs of the invention is not toxic to the mammalian host cell. FIG. 46 demonstrates that expression of the illustrated spike constructs is not toxic to the COS7 host cell.

Methods for transfecting, expressing, culturing, isolating and purifying recombinant proteins from mammalian cell cultures are known in the art. For example, the SARS spike constructs of the invention may be expressed in 293 cells. These cells may be cultured and transfected in static or monolayer cultures. For rapid large-scale production of SARS protein antigens in sufficient quantities for in vitro and in vivo evaluation, including immunogenicity studies, large-scale transient transfection of 293 (human embryonic kidney) cells may be used to obtain milligram quantities of the recombinant antigen(s). Alternatively, larger scale transfection of these cells may be performed with 293 cells in suspension culture. Preferably, the expressed SARS proteins are harvested from the transfected cells between 48 and 72 hours after transfection or even from 72 to 96 or more hours after transfection.

Where the host cells are transfected with truncated spike expression constructs, the expressed spike protein is secreted from the host cells and collected from the cell media. After concentration, the spike protein may be purified from the media using, for example, GNA lectin followed by DEAE and ceramic hydroxyapatite column chromatography.

Where the host cells are transfected with full length spike expression constructs, but rather is retained within the cells, and may be purified from triton X-100 detergent extracted cells. The full-length Spike protein can then be captured on GNA lectin, followed by hydroxyapatite and SP chromatography.

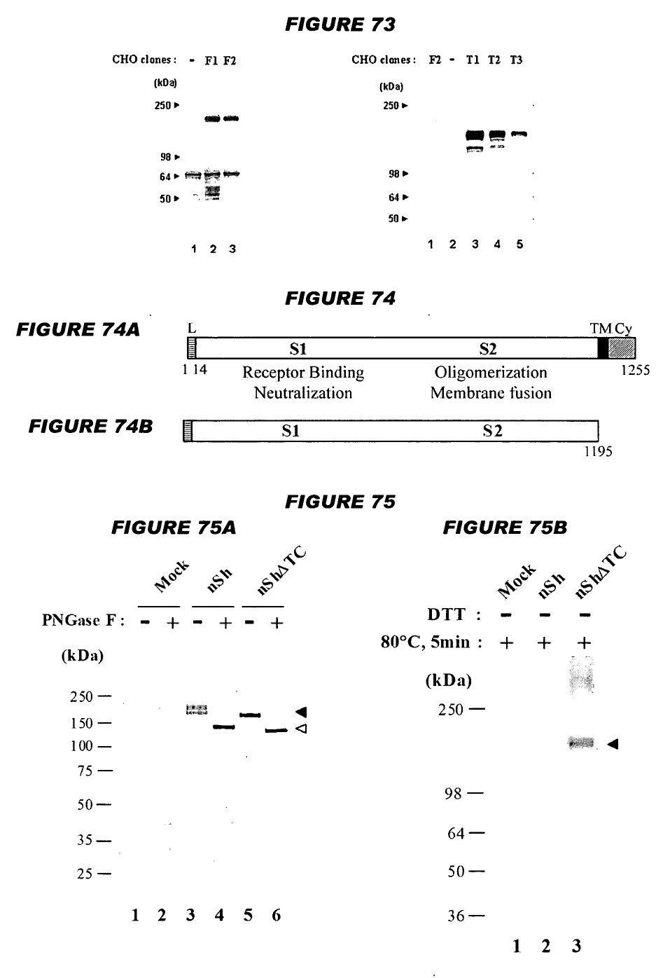

Chinese Hamster Ovary (CHO) or other eukaryotic (e.g., mammalian) cells that stably express the SARS viral antigens of the invention may also be derived (e.g. FIG. 73). Preferably, the cells are CHO cells, and these constructs will comprise one or more marker or selection genes in order to select for the desired CHO cells. In one embodiment, the constructs comprise a CMV enhancer/promoter, ampicillin resistance gene, and a fused DHFR and attenuated neomycin gene for selection purposes. Stable cell lines can then be produced using the neomycin selection system in CHOK-1 cells. Selected clones can then be sequenced to verify the integrity of the insert, and transient transfections can then be performed using Trans-LT1 polyamine transfection reagent (PanVera Corp., Madison, Wis.) to assess the expression level and also the integrity of the expressed protein by ELISA and western blot analysis.

Methods for derivation of CHO cells stably expressing the SARS viral antigens of the invention comprise the steps of transfection and primary screening with selective medium. Optionally, these steps are followed by subcloning to assure purity of cell lines. Cell culture supernatants can be assayed using an antigen capture ELISA to quantify expression levels at all stages of selection and amplification.

For full-length Spike expression constructs, methanol fixed cells can be screened for internal expression by immunofluorescent staining using a rabbit anti-SARS antibody. Successive measurements at the T75-flask stage of expansion can be employed to assure stability of expression levels. The molecular mass and integrity of the expressed proteins can be checked by PAGE both under native and reducing and denaturing conditions, followed by immunoprobing.

In one embodiment, the pCMV3 vectors expressing SARS-CoV Spike proteins in either full-length or truncated forms is introduced into CHOK-1 cells using the Trans-LT-1 reagent. On day one, 1×10⁶ cells are plated on 100 mm dishes in non-selective F12 media+10% Fetal Bovine Serum+4 mM Glutamine. On day two, the cells are transfected with a DNA:LT-1 mixture and the media then replaced with complete F12 media. Twenty-four to forty-eight hours later depending on the cell density, each 100 mm dish is split to 4-6 100 mm dishes. The medium is changed to complete selective media containing Geneticin (neomycin) at 500 μg/ml. All bovine serum used in these procedures is from TSE-free sources that meet current FDA standards. Twenty-four hours later the medium is changed to complete selective medium plus 500 ug/ml neomycin. Ten to fourteen days later, individual colonies are picked and transferred to 96 well plates and cultured in complete selective medium but without G418. When approximately 80% of the wells are confluent, twenty-four hour supernatants are screened by spike capture ELISA positive clones are transferred to twenty-four well plates. For the initial expression of full length Spike protein, methanol fixed cells will be screened by immunoflourescent staining using a rabbit anti-SARS antibody. After the low expressing cell lines have been eliminated and there are less than 20-30 cell lines, capture ELISA and westerns will be used to determine the expression level after cell lysis. A portion of each cell line will be pelleted, weighed and lysed in 1% triton lysis buffer containing MOPS, NaCl and MgCl₂ at the same ratio of cell weight to lysis buffer. After lysis the supernatant is collected and expression level is determined. Three to four clones producing the highest levels of spike protein in correct structure and conformation will be grown in three-liter bioreactors for expansion and adaptation to low serum suspension culture conditions for scale-up.

The antigen capture ELISA assay for the SARS spike protein can be performed as described in the art. A brief description of this assay follows. 96 well flat-bottom plates (Corning, Corning, N.Y.) are coated with 250 ng per well of purified immunoglobulin obtained from rabbit sera that were immunized with inactivated SARS virus. Between steps, the plates are washed in a buffer containing 16% NaCl and 1% Triton X100. 100 μL of supernatant or lysate samples (diluted in a buffer containing 100 mM NaPO₄, 0.1% Casein, 1 mM EDTA, 1% Triton X100, 0.5M NaCl and 0.01% Thiomersal, pH 7.5) are added and incubated for 2 hours at 37° C. Bound antigen is reacted against pooled SARS+ve serum or high affinity monoclonal antibody either human or mouse against SARS spike protein (1 hour incubation, 37° C.) and detected using appropriate species-specific peroxidase conjugated second antibody (30 minute incubation at 37° C.; TAGO, Burlingame, Calif.). The plates are developed for 15 minutes at room temperature using TMB substrate (Pierce, Rockford, Ill.) and the reaction stopped using 4N phosphoric acid. The plates are read at a wavelength of 450 nm and the concentration of protein per ml sample is derived from a standard curve (OD vs. protein concentration) based on serial dilutions of a known concentration of recombinant spike protein.

The immunoprobing analysis can also be performed following the standard methods described elsewhere in the art. A brief description follows. 10-20 μl of the sample is analyzed on 4-20% SDS PAGE under non-reducing/denaturing conditions with mild heating. The gels are run for 1.5-2.0 hours at 100V constant voltage. The proteins are then transferred onto nitrocellulose membranes (Millipore, Bedford, Mass.) for 45 min using the semidry western transfer system (BioRad, Hercules, Calif.) following the manufacturer's instructions. The membrane is then reacted against polyclonal anti-spike rabbit serum, followed by anti-rabbit Ig conjugated to Alexa 688 (Molecular Probes, Oregon). The blots are scanned using an infrared imaging system (LI-Cor, Inc., Lincoln, Nebr.).

The highest expressing candidate cell lines can be screened for spike protein expression and stability in small-scale (3 liter) suspension cultures. The candidate clone can be further evaluated for level of expression as well as integrity of expressed protein after amplification, and subsequently tested for expression stability in the absence of selection. The selected clones can also be tested for maintenance of the DNA sequence integrity of the integrated SARS spike protein gene. To quickly monitor the expression levels in small flask (T25 or T75) and in the three liter evaluation cultures, a lectin-based process (Gluvanthus Nivalis lectin) may be used to isolate SARS spike protein to a degree of purity that allows semi-quantitation and characterization of the protein in CHO supernatant. For full-length spike protein, it will be obtained from triton X-100 detergent extracted cells. Full-length Spike protein will be then captured on GNA lectin, followed by hydroxyapatite and SP chromatograph. Eluted protein is then characterized by: 1) polyacrylamide gel electrophoresis (PAGE) and Coomassie staining, 2) Immunoprobing with anti-SARS rabbit sera, 3) structural characterization using size exclusion chromatography (SEC), as well as mass spec analysis using MALDI-TOF.

Routes and methods of immunization of the vaccines of the invention are discussed in more detail in a section below. Examples 7 to 9 illustrate sample immunization protocols for the recombinant spike proteins.

Vaccine Testing

Prior to human administration, it is normal to test vaccines in animal models. A mouse model of SARS coronavirus infection is known (Subbarao et al. (2004) J Virol 78:3572-77), and other animals that may be used as models of infection and/or disease include ferrets and monkeys. Thus the invention provides a non-human animal that is infected by the SARS coronavirus, wherein the animal is preferably a ferret or a primate (e.g. a monkey or a macaque). The animal may be gnotobiotic. The animal is preferably not a cat (Felis domesticus). The animal may or may not display SARS disease symptoms e.g. ferrets (Mustela furo) show prominent pulmonary pathology after infection. See: Martina et al. (2003) Nature 425:915.

E. Polynucleotides Encoding the SARS Antigens of the Invention

The invention includes polynucleotides encoding for the SARS antigens of the invention. In addition, the invention includes polynucleotides which have been optimized for recombinant production (e.g. codon optimization) of the SARS antigens of the invention, including polynucleotides encoding for each of the SARS fusion constructs discussed above.

F. Viral Vector or Viral Particle Delivery of the SARS Antigens of the Invention

The antigens of the invention may be expressed in vivo or in vitro by polynucleotides encoding the antigens. Expression and delivery of the polynucleotides of the invention may be facilitated via viral vectors and/or viral particles.

Gene-based delivery systems derived from viruses, such as alphaviruses, are useful for the ex vivo and in vivo administration of heterologous genes, including one or more SARS genes, having therapeutic or prophylactic applications. These systems can also be used for the production of recombinant proteins derived from the SARS virus in cultured cells. Gene-based delivery systems of the invention include viral vectors (e.g., adenovirus vector, poxvirus vector, alphavirus vector) and non-viral nucleic acid vectors (e.g., DNA, RNA) encoding one or more SARS virus antigens. Polynucleotides encoding SARS virus antigen(s) are incorporated into the gene-based vaccines individually or in combination (e.g., as bicistronic constructs).

1. Alphavirus

Alphaviruses are members of Togaviridae family and share common structural and replicative properties. Sindbis virus (SIN) is the prototype virus for the molecular study of other alphaviruses, and together with Venezuelan equine encephalitis virus (VEE) and Semliki Forest virus (SFV), are the most widely utilized alphaviruses being developed into expression vectors for heterologous genes (Schlesinger and Dubensky (1999) Curr Opin. Biotechnol. 10:434-439; Schlesinger (2001) Expert Opin. Biol. Ther. 1:177-91).

Alphaviruses possess a relatively small single-stranded RNA genome of positive polarity, which is approximately 12 kb in length, capped and polyadenylated. The RNA interacts with viral capsid protein monomers to form nucleocapsids, which in turn, are surrounded by a host cell-derived lipid envelope from which two viral glycoproteins, E1 and E2, protrude forming “spike” trimers of heterodimeric subunits. Two open reading frames (ORFs) encode as polyproteins the enzymatic nonstructural replicase proteins (5′ ORF) and the virion structural proteins (3′ ORF). The structural polyprotein is translated from a highly abundant subgenomic mRNA, which is transcribed from a strong internal alphavirus promoter (Strauss and Strauss (1994) Microbiol. Rev. 58:491-562). Replication of the genome occurs exclusively within the host cell cytoplasm as RNA.

The most common alphavirus expression vectors have exploited both the positive-stranded nature and modular organization of the RNA genome. These vectors, termed “replicons” due to their property of self-amplification, permit insertion of heterologous sequences in place of the structural polyprotein genes, while maintaining the 5′- and 3′-end cis replication signals, the nonstructural replicase genes, and the subgenomic junction region promoter (Xiong et al. (1989) Science 243:1188-1191; Liljestrom (1991) Bio/Technology 9:1356-1361). Chimeric alphavirus vectors (and particles) from sequences derived from divergent virus families have also been described. (see, for example U.S. patent application Ser. No. 09/236,140; see also, U.S. Pat. Nos. 5,789,245, 5,842,723, 5,789,245, 5,842,723, and 6,015,694; as well as WO 95/07994, WO 97/38087 and WO 99/18226). Co-owned International Publication WO 02/099035, published Dec. 12, 2002 and incorporated by reference in its entirety herein, describes chimeric alphavirus molecules and modified alphavirus molecules having modified Biosafety Levels.

The absence of structural protein genes renders alphavirus replicon vectors defective, in that RNA amplification and high-level heterologous gene expression occurs within the target cell, but cell-to-cell spread of vector is not possible due to the inability to form progeny virions. Through the years, several synonymous terms have emerged that are used to describe alphavirus replicon particles. These terms include recombinant viral particle, recombinant alphavirus particle, alphavirus replicon particle and replicon particle. However, as used herein, these terms all refer to a virion-like unit containing an alphavirus-derived RNA vector replicon. Moreover, these terms may be referred to collectively as vectors, vector constructs or gene delivery vectors.

Packaging of replicon RNA into particles can be accomplished by introducing the replicon RNA into permissive cells (e.g., RNA or DNA transfection, or particle infection) that also contain one or more structural protein expression cassettes or “defective helper” constructs encoding the alphavirus structural proteins. These structural protein encoding constructs may themselves be introduced into the cells by transfection of either RNA or DNA, and most commonly retain the native alphavirus subgenomic promoter, as well as 5′- and 3′-end cis signals for co-amplification with the replicon, but are devoid of any replicase genes and the RNA packaging signal (Liljestrom (1991) Bio/Technology 9:1356-1361; Pushko et al. (1997) Virology 239:389-401; Polo et al. (1999) PNAS 96:4598-4603). Permanent cell lines that are stable transformed with constructs expressing the alphavirus structural proteins (e.g., packaging cell lines) offer a means to avoid transient transfection production methods (Polo et al. (1999) PNAS 96:4598-4603).

The present invention includes compositions and methods for the production of replication defective viral vector particles (e.g., alphavirus replicon particles) for use in the ex vivo and in vivo administration of heterologous genes encoding proteins having therapeutic or prophylactic application, including genes encoding for one or more SARS viral antigens.

In one aspect, the invention includes a method of producing replication defective viral vector particles (e.g., alphavirus replicon particles) comprising the steps of introducing at least one nucleic acid molecule comprising a viral vector (e.g., alphavirus replicon RNA) into immortalized cells of the present invention, under conditions that allow for complementation of the viral vector (e.g., alphavirus replicon RNA) and production of viral vector particles (e.g., alphavirus replicon particles), and isolating the viral vector particles from the cells or cell culture supernatants. In certain embodiments, the immortalized cells are grown in suspension, for example PERC.6 cells. In other embodiments, the methods are performed in large-scale volumes, for example, liter volumes or greater, such as for example in roller bottles, large flasks, Nunc Cell Factories, Corning Cell Cubes, fermentation vessels, etc).

In certain embodiments, the viral vector is an alphavirus replicon RNA that requires complementation by providing one or more alphavirus structural proteins in trans, within the immortalized cell. In such instances, the methods of complementation to produce alphavirus replicon particles may involve the introduction of one or more nucleic acids (e.g., RNA, DNA) encoding said alphavirus structural protein(s) (e.g., capsid and/or envelope glycoproteins) into the immortalized cells, either transiently or stably, and either concurrent with or prior to the introduction of the alphavirus replicon RNA. In certain embodiments, the alphavirus replicon RNA is introduced into the cell by transfection an in vitro transcribed RNA. In other embodiments, the alphavirus replicon RNA is introduced into the cell by transfection of a DNA (e.g., ELVIS), which is capable of transcribing within the cell, the replicon RNA. In yet other embodiments, the alphavirus replicon RNA is introduced into the cell by infection with a seed stock of alphavirus replicon particles. In certain embodiments, the nucleic acids encoding said alphavirus structural protein(s) are defective helper RNA or are DNA that can transcribe within the cell defective helper RNAs.

As discussed herein, “alphavirus RNA replicon vector”, “RNA replicon vector”, “replicon vector” or “replicon” refers to an RNA molecule that is capable of directing its own amplification or self-replication in vivo, within a target cell. To direct its own amplification, the RNA molecule should encode the polymerase(s) necessary to catalyze RNA amplification (e.g., alphavirus nonstructural proteins nsP1, nsP2, nsP3, nsP4) and also contain cis RNA sequences required for replication which are recognized and utilized by the encoded polymerase(s). An alphavirus RNA vector replicon should contain the following ordered elements: 5′ viral or cellular sequences required for nonstructural protein-mediated amplification (may also be referred to as 5′ CSE, or 5′ cis replication sequence, or 5′ viral sequences required in cis for replication, or 5′ sequence which is capable of initiating transcription of an alphavirus), sequences which, when expressed, code for biologically active alphavirus nonstructural proteins (e.g., nsP1, nsP2, nsP3, nsP4), and 3′ viral or cellular sequences required for nonstructural protein-mediated amplification (may also be referred as 3′ CSE, or 3′ viral sequences required in cis for replication, or an alphavirus RNA polymerase recognition sequence). The alphavirus RNA vector replicon also should contain a means to express one or more heterologous sequence(s), such as for example, an IRES or a viral (e.g., alphaviral) subgenomic promoter (e.g., junction region promoter) which may, in certain embodiments, be modified in order to increase or reduce viral transcription of the subgenomic fragment, or to decrease homology with defective helper or structural protein expression cassettes, and one or more heterologous sequence(s) to be expressed. Preferably the heterologous sequence(s) comprises a protein-encoding gene, which is the 3′ proximal gene within the vector replicon. And preferably the replicon further comprises a polyadenylate tract.

As discussed herein, “recombinant Alphavirus Particle”, “alphavirus replicon particle” and “replicon particle” refers to a virion-like unit containing an alphavirus RNA vector replicon. Generally, the recombinant alphavirus particle comprises one or more alphavirus structural proteins, a lipid envelope and an RNA vector replicon. Preferably, the recombinant alphavirus particle contains a nucleocapsid structure that is contained within a host cell-derived lipid bilayer, such as a plasma membrane, in which one or more alphaviral envelope glycoproteins (e.g., E2, E1) are embedded. The particle may also contain other components (e.g., targeting elements such as biotin, other viral structural proteins or portions thereof, hybrid envelopes, or other receptor binding ligands), which direct the tropism of the particle from which the alphavirus was derived. Generally the interaction between alphavirus RNA and structural protein(s) necessary to efficiently form a replicon particle or nucleocapsid may be an RNA-protein interaction between a capsid protein and a packaging signal or packaging sequence contained within the RNA.

“Alphavirus packaging cell line” refers to a cell which contains one or more alphavirus structural protein expression cassettes and which produces recombinant alphavirus particles (replicon particles) after introduction of an alphavirus RNA vector replicon, eukaryotic layered vector initiation system, or recombinant alphavirus particle. The parental cell may be of mammalian or non-mammalian origin. Within preferred embodiments, the packaging cell line is stably transformed with the structural protein expression cassette(s).

“Defective helper RNA” refers to an RNA molecule that is capable of being amplified and expressing one or more alphavirus structural proteins within a eukaryotic cell, when that cell also contains functional alphavirus nonstructural “replicase” proteins. The alphavirus nonstructural proteins may be expressed within the cell by an alphavirus RNA replicon vector or other means. To permit amplification and structural protein expression, mediated by alphavirus nonstructural proteins, the defective helper RNA molecule should contain 5′-end and 3′-end RNA sequences required for amplification, which are recognized and utilized by the nonstructural proteins, as well as a means to express one or more alphavirus structural proteins. Thus, an alphavirus defective helper RNA should contain the following ordered elements: 5′ viral or cellular sequences required for RNA amplification by alphavirus nonstructural proteins (also referred to elsewhere as 5′ CSE, or 5′ cis replication sequence, or 5′ viral sequences required in cis for replication, or 5′ sequence which is capable of initiating transcription of an alphavirus), a means to express one or more alphavirus structural proteins, gene sequence(s) which, when expressed, codes for one or more alphavirus structural proteins (e.g., C, E2, E1), 3′ viral or cellular sequences required for amplification by alphavirus nonstructural proteins (also referred to as 3′ CSE, or 3′ viral sequences required in cis for replication, or an alphavirus RNA polymerase recognition sequence), and a preferably a polyadenylate tract. Generally, the defective helper RNA should not itself encode or express in their entirety all four alphavirus nonstructural proteins (nsP1, nsP2, nsP3, nsP4), but may encode or express a subset of these proteins or portions thereof, or contain sequence(s) derived from one or more nonstructural protein genes, but which by the nature of their inclusion in the defective helper do not express nonstructural protein(s) or portions thereof. As a means to express alphavirus structural protein(s), the defective helper RNA may contain a viral (e.g., alphaviral) subgenomic promoter which may, in certain embodiments, be modified to modulate transcription of the subgenomic fragment, or to decrease homology with replicon RNA, or alternatively some other means to effect expression of the alphavirus structural protein (e.g., internal ribosome entry site, ribosomal readthrough element). Preferably an alphavirus structural protein gene is the 3′ proximal gene within the defective helper. In addition, it is also preferable that the defective helper RNA does not contain sequences that facilitate RNA-protein interactions with alphavirus structural protein(s) and packaging into nucleocapsids, virion-like particles or alphavirus replicon particles. A defective helper RNA is one specific embodiment of an alphavirus structural protein expression cassette.

Alphavirus for use in the invention may be grown in any one of the cell lines discussed above as suitable for the SARS virus.

Alphavirus replicon particles may be produced according to the present invention by using the above cell lines (e.g., immortalized cell lines) and a variety of published and accepted alphavirus vector methodologies. Such methodologies include, for example, transient packaging approaches, such as the co-transfection of in vitro transcribed replicon and defective helper RNA(s) (Liljestrom, Bio/Technology 9:1356-1361, 1991; Bredenbeek et al., J. Virol. 67:6439-6446, 1993; Frolov et al., J. Virol. 71:2819-2829, 1997; Pushko et al., Virology 239:389-401, 1997; U.S. Pat. Nos. 5,789,245 and 5,842,723) or co-transfection of plasmid DNA-based replicon and defective helper construct(s) (Dubensky et al., J. Virol. 70:508-519, 1996), as well as introduction of alphavirus structural protein expression cassettes (e.g., DNA-based defective helper) into immortalized cell lines of the present invention to create stable packaging cell lines (PCL) (Polo et al., PNAS 96:4598-4603, 1999; U.S. Pat. Nos. 5,789,245, 5,842,723, 6,015,694; WO 97/38087, WO 99/18226, WO 00/61772, and WO 00/39318). Stable packaging cell lines may then be utilized for alphavirus replicon particle production. The PCL may be transfected with in vitro transcribed alphavirus replicon RNA, transfected with a plasmid DNA-based replicon (e.g., ELVIS vector), or infected with a seed stock of alphavirus replicon particles, and then incubated under conditions and for a time sufficient to produce progeny alphavirus replicon particles in the culture supernatant. In addition, progeny replicon particles can subsequently be passaged in additional cultures of naive PCL by infection, resulting in further expansion and commercial scale preparations. Importantly, by using defective helper RNA or stable PCL based on the “split” structural gene configuration, these replicon particle stocks may be produced free from detectable contaminating RCV.

Following harvest, crude culture supernatants containing the chimeric alphavirus replicon particles may be clarified by passing the harvest through a filter (e.g., 0.2 uM, 0.45 uM, 0.65 uM, 0.8 uM pore size). Optionally, the crude supernatants may be subjected to low speed centrifugation prior to filtration to remove large cell debris. Within one embodiment, an endonuclease (e.g., Benzonase, Sigma #E8263) is added to the preparation of alphavirus replicon particles before or after a chromatographic purification step to digest exogenous nucleic acid. Further, the preparation may be concentrated prior to purification using one of any widely known methods (e.g., tangential flow filtration). Crude or clarified alphavirus replicon particles may be concentrated and purified by chromatographic techniques (e.g., ion exchange chromatography, size exclusion chromatography, hydrophobic interaction chromatography, affinity chromatography), such as those described in WO01/92552, incorporated by reference in its entirety herein. Two or more such purification methods may be performed sequentially.

### Example of Alphavirus Replicon Particles Encoding SARS Virus Spike (S) Antigen

The invention includes compositions and methods for the production of replication defective viral vector particles (e.g., alphavirus replicon particles) for use in the ex vivo and in vivo administration of heterologous genes encoding proteins having therapeutic or prophylactic application, including genes encoding for one or more SARS viral antigens.

The following example illustrates a method of preparing alphavirus replicon particles encoding SARS virus spike (s) antigen.

The SARS virus spike gene can be incorporated into alphavirus replicon particles derived from a variety of alphavirus, such as Sindbis virus, Semliki Forest virus (U.S. Pat. No. 5,739,026), Venezuelan equine encephalitis virus (U.S. Pat. No. 6,531,135), and replicon particle chimeras derived from more than one alphavirus (U.S. Pat. No. 6,376,236, WO 02/99035). In addition, the SARS virus spike gene can be incorporated in its entirety (encoding full-length spike protein) or in a modified form that includes, for example, sequence deletions or truncations, such that the encoded a spike protein is of less than full-length (e.g., C-terminal truncation of one or more (e.g. at least 1, 2, 3, 4, 5, 6, 7, 8, 9, 10, 15, 20, 25, 30 etc.) amino acids, deleted of transmembrane region and cytoplasmic tail).

For example, the spike gene may be cloned as a full-length gene into the VCR-chim2.1 vector (WO 02/99035) by standard RT-PCR conditions or by standard subcloning from one of the other plasmids described herein, using commercially available restriction endonucleases. For the reverse transcription step in standard RT-PCR, the Superscript pre-amplification kit (Invitrogen™) and the primer SEQ ID NO: 7325 (sp-RT-R) are used:

For the amplification step, the cDNA polymerase advantage kit (Clonetech) and two primers Sp-F-BbvCI (SEQ ID NO: 7326) and Sp-R-NotI (SEQ ID NO: 7327) are used:

The forward primer is designed to contain the ccacc sequence (Kozak, 1991 JBC 19867-70) in front of the ATG codon to optimize translation efficiency of the spike gene. Also, the forward primer contains the BbvCI restriction site and the reverse primer contains the NotI restriction site for subsequent cloning of the PCR amplified gene.

The PCR product is purified using the QIAquick Nucleotide Removal kit (QIAgen), digested with BbvCI and NotI, gel purified with QIAquick Gel Extraction kit (QIAgen), and ligated to plasmid VCR-Chim2.1 pre-digested with the same enzymes. Clones containing the SARS spike sequence are verified by sequencing and the new construct is called VCR-Chim2.1-SARSspike.

To generate VEErep/SINenv-SARSspike replicon particles the plasmids VCR-Chim2.1-SARSspike, VCR-DH-Scap (WO 02/99035), and VCR-DH-Sglyd1160 (WO 02/99035) are linearized with the restriction enzyme PmeI and used for in vitro transcription as described previously (Polo et al. 1999, PNAS 96: 4598-603; WO02/99035). The transcripts are co-transfected into BHK cells as previously described (Polo et al., 1999, ibid.; WO02/99035). The transfected cells are incubated at 34° C., the supernatants collected at 20 and 30 hrs post-electroporation, clarified by centrifugation, and purified by chromatography as previously described (WO 01/92552).

Expression of the SARS spike protein from the replicon particle vector is verified by infecting BHK cells overnight with purified VEErep/SINenv-SARSspike or VEErep/SINenv-GFP (WO 02/99035) replicon particles. In addition, BHK cells also were transfected in parallel with in vitro transcribed VCR-Chim2.1-SARSspike replicon RNA. At 16 hrs post-infection and transfection cells are lysed and a sample of the lysate analyzed by western blot using an antibody that recognizes SARS virus spike protein. The proteins on the gel are stained or transferred to a membrane for Western blot analysis with sera from convalescent patients or alternatively murine or rabbit antisera generated against SARS virus. VEErep/SINenv-SARSspike replicon particles are administered to the vaccine recipient (e.g., rodent, non-human primate, human) as described elsewhere in the present invention.

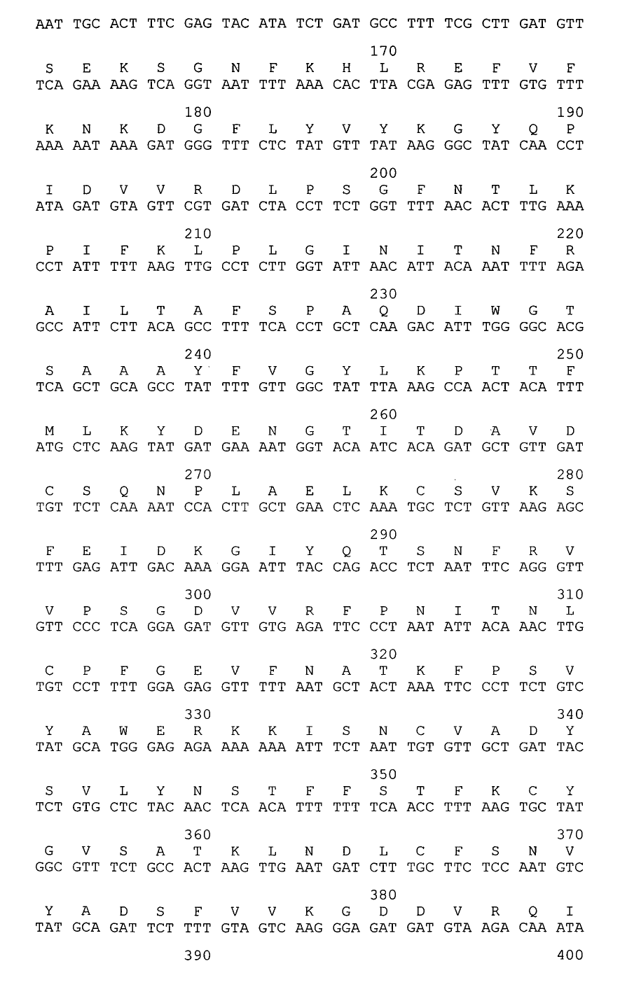

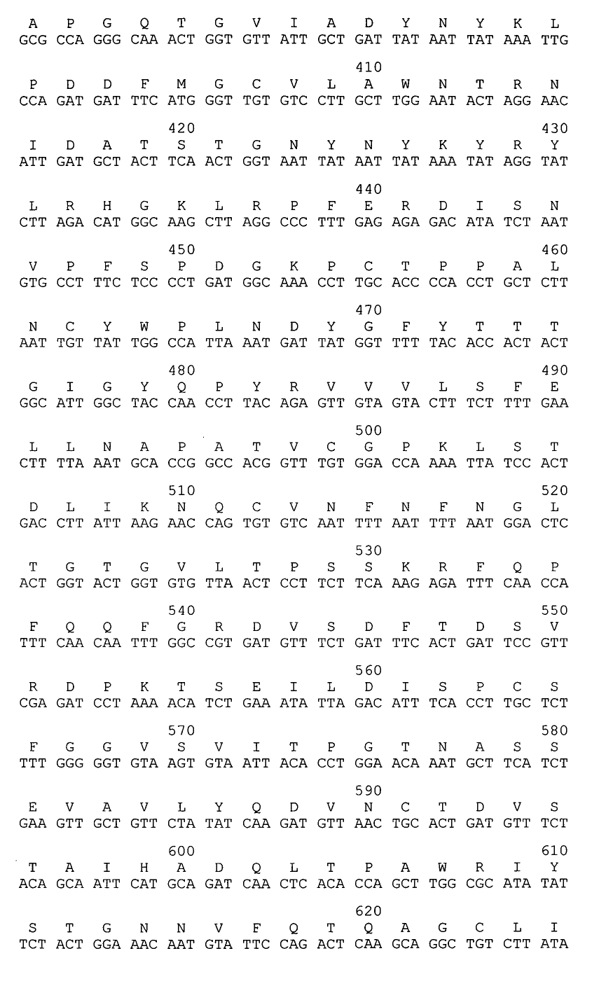

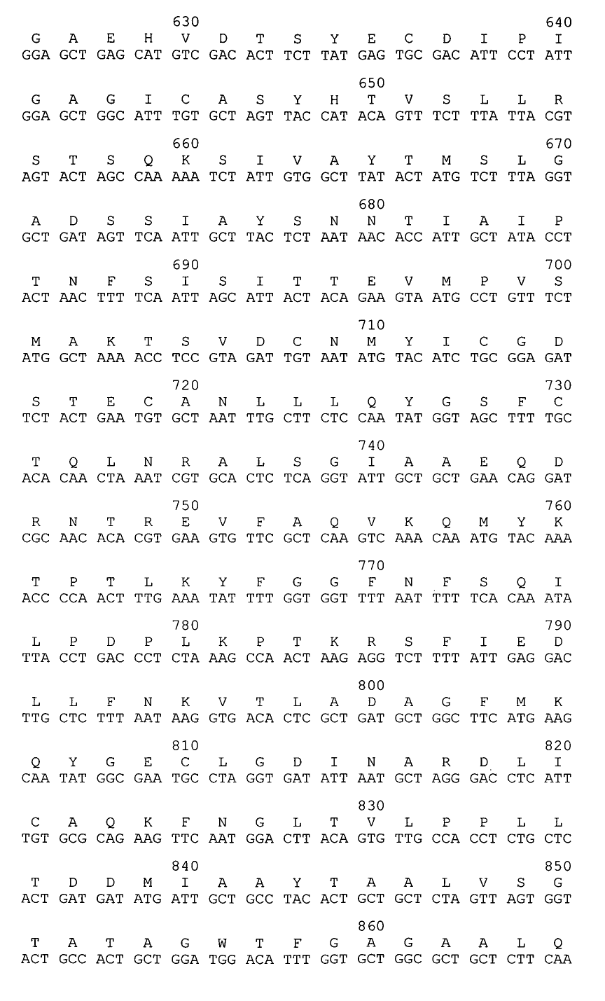

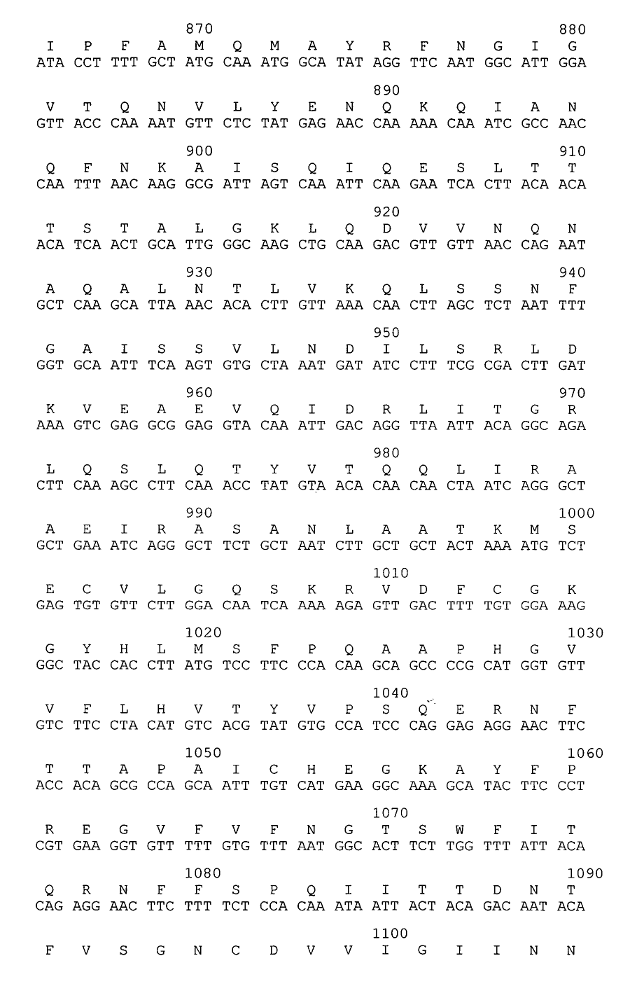

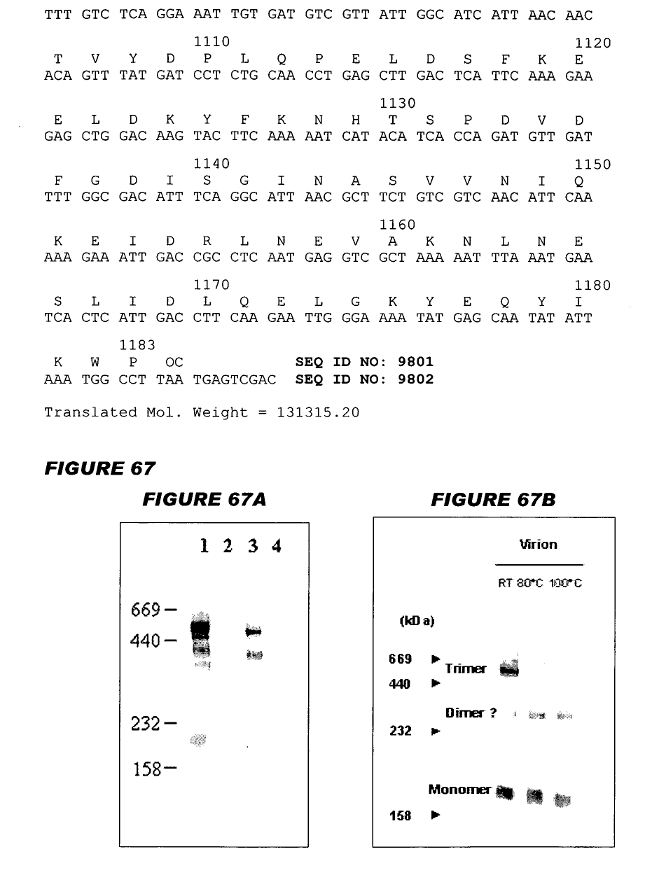

FIG. 67 shows data from western blot analysis performed under non-reducing conditions, using a SARS virus specific rabbit polyclonal antisera. The western data demonstrate that not only is SARS spike protein expressed in cells infected with alphavirus replicon particles or transfected with replicon RNA, but the predominant form of spike is that of a homotrimer (FIG. 67A). Similar homotrimeric association of the spike protein was observed in western blots of SARS virions purified from SARS virus infected VERO cell supernatants, and this homotrimer is heat labile, as indicated by the dissociation into monomeric forms at 80° C. and 100° C. (FIG. 67B).

To further characterize SARS Spike protein expression and processing following expression from alphavirus replicon vectors, BHK-21 cells were infected with alphavirus replicon particles expressing the full-length Spike. At 6 hr post-infection with an MOI of 5, infected cells were labeled for 1 hr with L-[³⁵S]methionine/cysteine and chased for the indicated time. The [³⁵S]-labeled spike protein was immunoprecipitated by anti-SARS rabbit serum and digested with Endo-H. Both digested and undigested proteins were analysed by 4% polyacrylamide-SDS PAGE under reducing conditions. As shown in FIG. 55, the full-length spike protein is synthesized as an Endo-H sensitive high mannose glycoprotein (gp170, an ER form) that undergoes modification to an Endo-H resistant glycoprotein with complex oligosaccharides (gp180, a Golgi form). The conversion of gp170 into the gp180 form takes place within 2 hr.

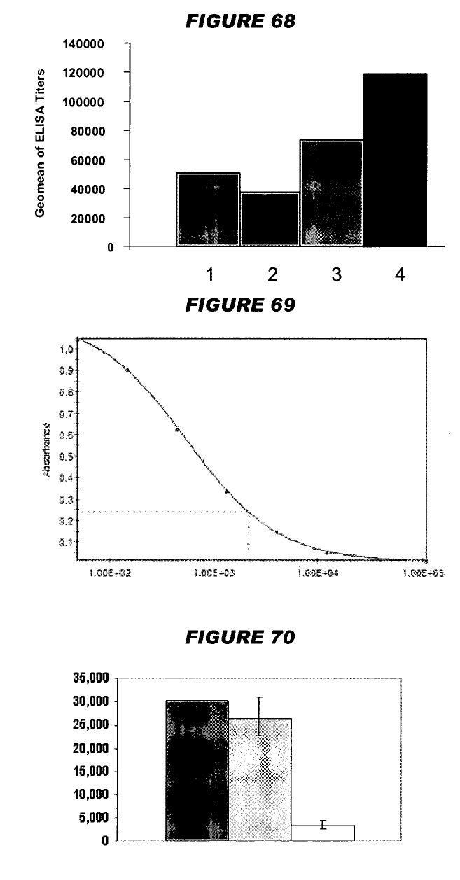

Alphavirus replicon particles expressing one or more SARS proteins (e.g., VEErep/SINenv-SARSspike replicon particles) are administered to the vaccine recipient in order to induce a SARS specific immune response (e.g., rodent, ferret, non-human primate, human) as described elsewhere in the present invention. Immunization may be performed through a variety of routes, including for example, intramuscular, subcutaneous, intradermal, and intranasal. In additon, the alphavirus replicon particles may be used alone or in combination (e.g., “prime-boost”) with other vaccine approaches of the present invention, or alternatively the alphavirus replicon particles may co-express antigen from other respiratory pathogens or be co-administered in combination with alphavirus replicon particles expressing antigens from other respiratory pathogens (e.g., influenza virus, parainfluenza virus, respiratory syncytial virus, human metapneumovirus). For example, the induction of anti-spike protein antibodies in animals immunized IM with VEErep/SINenv-SARSspike replicon particles was demonstrated in mice (FIG. 68). These mouse studies also included addtional vaccine groups for comparison, including the inactivated SARS virus and recombinant truncated spike protein vaccines describe elsewhere herein, as well as plasmid DNA used as a prime, followed by alphavirus replicon particles as a boost. The data clearly show very potent immune responses for all vaccine groups, including the alphavirus replicon particle group. It should be noted that the level of antibody induced by the inactivated SARS virus vaccine used in these experiments has been shown to be protective in a SARS virus animal challenge model.

Similarly, genes encoding other SARS virus antigens (e.g., nucleocapsid protein, membrane glycoprotein) are cloned into alphavirus replicon vectors, either individually or in combination, to generate alphavirus replicon particles according to the teachings of the present invention and using standard molecular biology techniques.

### Example of Alphavirus-Based Plasmid DNA Expressing SARS Virus Spike (S)

The invention includes preparation of plasmid DNA expressing a SARS virus antigen for prophylactic or therapeutic immunization against SARS virus infection. In one embodiment, the SARS viral antigen is a spike (S) protein. In one embodiment, the plasmid DNA is alphavirus-based.

The following example illustrates one method for preparing an alphavirus-based plasmid DNA expressing SARS virus spike (S).

SARS spike gene can be delivered using any of the alphavirus-based plasmid DNA replicons such as ELVS (Dubensky et al, 1996 J Virol. 70: 508-19), SINCP (WO 01/81609), or VCP (PCT WO 02/99035).

For example, the SARS spike gene is cloned into SINCP using the standard RT-PCR techniques. The oligo Sp-RT-R is used for the reverse transcription step with the Superscript pre-amplification kit (Invitrogen). For the amplification step, the cDNA polymerase advantage kit (Clonetech) with the Sp-R-NotI and Sp-F-XhoI (SEQ ID NO: 7328) primers is used.

The Sp-F-XhoI primer was designed to contain the ccacc sequence in front of the ATG codon to optimize translation efficiency (Kozak 1991, ibid) of the spike gene. Also, the primer contains the XhoI restriction site for the subsequent cloning of the PCR amplified gene.

The PCR product is purified using the QIAquick Nucleotide removal kit, digested with XhoI and NotI, gel purified with QIAquick Gel Extraction kit, and ligated to plasmid SINCP pre-digested with the same enzymes. Clones containing the SARS spike sequence are verified by sequencing and the new construct is called SINCP-SARSspike.

Expression of the SARS spike gene is verified by transient transfection of BHK cells with 2 μg of either plasmid DNA SINCP-SARSspike or SINCP pre-incubated for 5 minutes with 5 μl of TransIT Polyamine reagent (Mirrus) in low serum medium Optimem (Life Technologies). At 48 hrs pos-transfection cells are lysed and a sample of the lysate is run on 8% SDS-PAGE. The proteins on the gel are either stained or transferred to a membrane for Western blot analysis with sera from convalescent patients, or alternatively with sera from mouse or rabbits.

SINCP-SARSspike plasmid replicon is administered to the vaccine recipient (e.g., rodent, non-human primate, human) as a formulated or unformulated plasmid vaccine, alone or in combination (e.g., “prime-boost”) with other vaccines of the present invention, as described elsewhere herein.

Similarly, genes encoding other SARS virus antigens (e.g., nucleocapsid protein, membrane glycoprotein) are cloned into alphavirus plasmid replicon vectors.

2. Plasmid Expression Vectors

### Example of Plasmid DNA Expressing SARS Virus Spike (S)

The following example illustrates a method for preparing plasmid DNA expressing SARS virus spike (s).

The SARS virus spike antigen also may be delivered using other plasmid DNA expression vectors (sometimes referred to as “conventional” DNA vaccines), based on a polymerase II promoter, such as, for example, a CMV promoter. A DNA vaccine of the spike antigen gene induces an antibody response in mice (Zhao et al. (2004) Acta Biochim et Biophysica Sinica 36:37-41), and has been found to induce viral neutralization and protective immunity in mice (Yang et al. (2004) Nature 428:561-564), particularly when truncated at the C-terminus.

For example, the SARS spike gene is cloned into pCMVKm2 (Zur Megede et al., J. Virol., 74:2628-2635, 2000; SEQ ID NO: 9923) using standard RT-PCR techniques. The oligo Sp-RT-R is used for the reverse transcription step with the Superscript pre-amplification kit (Invitrogen). For the amplification step, the cDNA polymerase advantage kit (Clonetech) is used with primers Sp-F-EcoRI (SEQ ID NO: 7329) and Sp-R-XbaI (SEQ ID NO: 7330).

The forward primer was designed to contain the CCACC (SEQ ID NO: 7331) sequence in front of the ATG codon to optimize translation efficiency (Kozak 1991, ibid.) of the spike gene. Also, the forward primer contains the EcoRI restriction site and the reverse primer contains the XbaI restriction site for the subsequent cloning of the PCR amplified gene.

The PCR product is purified using the QIAquick Nucleotide Removal kit, digested with XhoI and NotI, gel purified with QIAquick Gel Extraction kit, and ligated to plasmid pCMVKm2 pre-digested with the same enzymes. Clones containing the SARS spike sequence are verified by sequencing and the new construct is called pCMVKm2-SARSspike.

Expression of the SARS spike gene is verified by transient transfection of BHK or 293 cells with 2 μg of either plasmid DNA pCMVKm2-SARSspike or pCMVKm2 pre-incubated for 5 minutes with 5 μl of TransIT Polyamine reagent (Mirrus) in low serum medium Optimem (Life Technologies). At 48 hrs pos-transfection cells are lysed and a sample of the lysate is run on 8% SDS-PAGE. The proteins on the gel are either stained or transferred to a membrane for Western blot analysis with sera from convalescent patients, or alternatively using mouse or rabbit antisera.

Plasmid pCMVKm2-SARSspike is administered to the vaccine recipient (e.g., rodent, non-human primate, human) as a formulated or unformulated plasmid vaccine, as described elsewhere in the present invention.

Similarly, genes encoding other SARS virus antigens (e.g., nucleocapsid protein, membrane glycoprotein) are cloned into plasmid expression vectors

3. Virus-Like Particles Comprising SARS Antigens

The SARS viral antigens of the invention may be formulated into Virus Like Particles (“VLPs”). The invention thus includes virus-like particles (or VLPs) comprising one or more SARS viral antigens. Preferably, the VLPs comprise one or more SARS viral antigens selected from the group consisting of Spike (S), nucleocapsid (N), membrane (M) and envelope (E). Preferably, the VLPs comprise at least M and E.

The VLPs of the invention comprise at least one particle-forming polypeptide. Said particle-forming polypeptide is preferably selected from a Coronavirus structural protein. In one embodiment, the particle-forming polypeptide is selected from one or more SARS viral antigens. In another embodiment, the particle-forming polypeptide is selected from the structural protein of a non-SARS Coronavirus, such as, for example, Mouse Hepatitis Virus.

VLPs can be formed when viral structural proteins are expressed in eukaryotic or prokaryotic expression systems. Upon expression, the structural proteins self-assemble to form particles. Alternatively, viral structural proteins may be isolated from whole virus and formulated with phospholipids. Such viral structural proteins are referred to herein as “particle-forming polypeptides”. VLPs are not infectious because no viral genome is present, however, these non-replicating, virus capsids mimic the structure of native virions.

Due to their structure, VLPs can display a large number of antigenic sites on their surface (similar to a native virus). VLPs offer an advantage to live or attenuated vaccines in that they are much safer to both produce and administer, since they are not infectious. VLPs have been shown to induce both neutralizing antibodies as well as T-cell responses and can be presented by both class I and II MHC pathways.

Previous work creating VLPs from coronavirus indicates that E and M proteins along may be sufficient for coronavirus VLP formation. See Fischer et al., J. Virol. (1998) 72:7885-7894 and Vennema et al. EMBO J. (1996) 15:2020-2028.

Chimeric VLPs comprising particle-forming polypeptides or portions thereof from non-SARS Coronaviruses are also included in the invention. Such particle-forming polypeptides may comprise a full length polypeptide from a non-SARS Coronavirus. Alternatively, a particle-forming fragment may be used.

In one embodiment, a fragment of a non-SARS particle-forming polypeptide and a fragment of a SARS viral antigen are fused together. For instance, such chimeric polypeptides may comprise the the endodomain and transmembrane domain of a non-SARS particle-forming polypeptide and the ectodomain of a SARS viral antigen. In one example, the VLPs of the invention comprise a chimeric spike protein comprising an endodomain and transmembrane domain of the spike protein of Mouse Hepatitis Virus (MHV) and the chimeric spike protein further comprises the ectodomain of the SARS spike protein. Such VLPs may further comprise Coronavirus M and E proteins. Said M and E proteins may be selected from any coronavirus, including Mouse Hepatitis Virus (MHV) or SARS. Sample sequences of S, M and E proteins of MHV are included in the figures, supra.

Chimeric spike proteins derived from the ectodomain of feline infectious peritonitis virus (FIPV) spike protein fused to the endo and transmembrane domains of MHV spike protein have been previously disclosed. See WO 98/49195 and WO 02/092827. In these chimeric VLP structures, the capsid structure of the VLPs is formed by the M and E protein of MHV. The chimeric spike protein provides for the surface exposure of the ectodomain of the FIPV spike protein.

As used herein, the term “virus-like particle” or “VLP” refers to a non-replicating, empty virus shell. VLPs are generally composed of one or more viral proteins, such as, but not limited to those proteins referred to as capsid, coat, shell, surface and/or envelope proteins, or particle-forming polypeptides derived from these proteins. VLPs can form spontaneously upon recombinant expression of the protein in an approrpirate expression system. Alternatively, viral structural proteins may be isolated from whole virus and formulated with phospholipids. Methods for producing particular VLPs are known in the art and discussed more fully below. The presence of VLPs in a composition can be detected using conventional techniques known in the art, such as by electron microscopy, x-ray crystallography, and the like. See, e.g., Baker et al., Biophys. J. (1991) 60:1445-1456; Hagensee et al., J. Virol. (1994) 68:4503-4505. For example, cryoelectron microscopy can be performed on vitrified aqueous samples of the VLP preparation in question, and images recorded under appropriate exposure conditions.

The phrase “particle-forming polypeptide” includes a full-length or near full-length viral protein, as well as a fragment thereof, or a viral protein with internal deletion, which has the ability to form VLPs under conditions that favor VLP formation. Accordingly, the polypeptide may comprise the full-length sequence, fragments, truncated and partial sequences, as well as analogs and precursor forms of the reference molecule. The term therefore includes deletions, additions and substitutions to the sequence, so long as the polypeptide retains the ability to form a VLP. Thus, the term includes natural variations of the specified polypeptide since variations in coat proteins often occur between viral isolates. The term also includes deletions, addition and substitutions that do not naturally occur in the reference protein, so long as the protein retains the ability to form a VLP.

Preferred substitutions are those which are conservative in nature, i.e., those substitutions that take place within a family of amino acids that are related in their side chains. Specifically, amino acids are generally divided into four families: (1) acidic: aspartate and glutamate; (2) basic: lysine, arginine, and histidine; (3) non-polar: alanine, valine, leucine, isoleucine, proline, phenylalanine, methionine, tryptophan; and (4) uncharged polar: glycine, asparagine, glutamine, cystine, serine, theronine, tyrosine. Phenylalanine, tryptophan, and tyrosine are sometimes classified as aromatic amino acids. For example, it is reasonably predictable that an isolated replacement of leucine with isoleucine or valine, an asparate with a glutamate, a threonine with a serine, or a similar conservative replacement of an amino acid with a structurally related amino acid, will not have a major effect on the biological activity. Proteins having substantially the same amino acid sequence as the reference molecule, but possessing minor amino acid substitutions that do not substantially affect the immunogenicity of the protein, are therefore within the definition of the reference polypeptide.

The VLPs of the invention can be formed from any viral protein, particle-forming polypeptide derived from the viral protein, or combination of viral proteins or fragments thereof, that have the capability of forming particles under appropriate conditions. The requirements for the particle-forming viral proteins are that if the particle is formed in the cytoplasm of the host cell, the protein must be sufficiently stable in the host cell in which it is expressed such that formation of virus-like structures will result, and that the polypeptide will automatically assemble into a virus-like structure in the cell of the recombinant expression system used. If the protein is secreted into culture media, conditions can be adjusted such that VLPs will form. Furthermore, the particle-forming protein should not be cytotoxic in the expression host and should not be able to replicate in the host in which the VLP will be used.

Preferred particle-forming polypeptides include coronavirus M and E proteins, preferably SARS M and E proteins.

Methods and suitable conditions for forming particles from a wide variety of viral proteins are known in the art. VLPs have been produced, for example from proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO 03/024480, WO 03/024481, and Niikura et al., Virology (2002) 293:273-280; Lenz et al., J. Immunology (2001) 5246-5355; Pinto, et al., J. Infectious Diseases (2003) 188:327-338; and Gerber et al., J. Virology (2001) 75(10):4752-4760.

As explained above, VLPs can spontaneously form when the particle-forming polypeptide of interest is recombinantly expressed in an appropriate host cell. Thus, the VLPs for use in the present invention may be prepared using recombinant techniques, well known in the art. In this regard, genes encoding the particle-forming polypeptide in question can be isolated from DNA libraries or directly from cells and tissues containing the same, using known techniques. The genes encoding the particle-forming polypeptides can also be produced synthetically, based on the known sequences. The nucleotide sequence can be designed with the appropriate codons for the particular amino sequence desired. In general, one will select preferred codons for the intended host in which the sequence will be expressed (e.g. human codons for human DNA vaccines). The complete sequence is generally assembled from overlapping oligonucleotides prepared by standard methods and assembled into a complete coding sequence. See., e.g., Edge, Nature (1981) 292:756; Nambair et al. Science (1984) 223:1299; Jay et al., J. Biol. Chem. (1984) 259:6311.

Once the coding sequences for the desired particle-forming polypeptides have been isolated or synthesized, they can be cloned into any suitable vector or replicon for expression. Numerous cloning vectors are known to those of skill in the art, and the selection of an appropriate cloning vector is a matter of choice. See, generally, Sambrook et al. The vector is then used to transform an appropriate host cell. Suitable expression systems include, but are not limited to, bacterial, mammalian, bacuolvirus/insect, vaccinia, Semliki Forest virus (SFV), yeast, and Xenopus expression systems, well known in the art.

A number of cell lines suitable for use as host cells for producing the VLPs of the invention are known in the art. Suitable mammalian cell lines include, but are not limited to, Chinese Hamster Ovary (CHO) cells, HeLa cells, baby hamster kidney (BHK) cells, monkey kidney cells (COS), human hepatocellular carcinoma cells (e.g., Hep G2), Madin-Darby bovine kidney (“MDBK”) cells, as well as others. Mammalian sources of cells include, but are not limited to, human or non-human primate (e.g., MRC-5 (ATCC CCL-171), WI-38 (ATCC CCL-75), HUH, human embryonic kidney cells (293 cells, typically transformed by sheared adenovirus type 5 DNA), VERO cells from monkey kidneys (including, for example COS7 cells), horse, cow (e.g., MDBK cells), sheep, dog (e.g., MDCK cells from dog kidneys, ATCC CCL34 MDCK (NBL2) or MDCK 33016, deposit number DSM ACC 2219 as described in WO 97/37001), cat, and rodent (e.g., hamster cells such as BHK21-F, HKCC cells, or Chinese hamster ovary cells (CHO cells)), and may be obtained from a wide variety of developmental stages, including for example, adult, neonatal, fetal, and embryo.

Bacterial hosts suitable for production of VLPs of the invention include E. coli, Bacillus subtilis, and Streptoccocus spp. Yeast hosts suitable for production of VLPs of the invention include Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells suitable for production of VLPs of the invention (i.e., via baculovirus expression vectors) include Aedes aegypti, Autographa californica, Bombyx mori, Drosophila melanogaster, Spodptera frugiperda, and Trichoplusia ni.

Viral vectors can be used for the production of particles in eukaryotic cells, such as those derived from the pox family of viruses, including vaccinia virus and avian poxvirus. Additional, vaccinia based infection/transfection systems, such as those as described in Tomei et al., J. Virol (1993) 67:4017-4026 and Selby et al., J. Gen. Virol. (1993) 74:1103-1113, can also be used to generate the VLPs of the invention. In this system, cells are first transfected in vitro with a vaccinia virus recombinant that encodes the bacteriophage T7 RNA polymerase. This polymerase only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA which is then translated into protein by the host translation machinery. The method provides for high level, transient, cytoplasmic production of large quantities of RNA and its translation products.

Depending on the expression system and host selected, the VLPs are produced by growing host cells transformed by an expression vector under conditions whereby the particle-forming polypeptide is expressed and VLPs can be formed. The selection of the appropriate growth conditions is within the skill of the art. If the VLPs are formed intracellularly, the cells are then disrupted, using chemical, physical or mechanical means, which lyse the cells yet keep the VLPs substantially intact. Such methods are known the those of skill in the art and are described in, e.g., Protein Purification Applications: A Practical Approach, (E. L. V. Harris and S. Angal, Eds., 1990).

The particles are then isolated using methods that preserve the integrity thereof, such as by gradient centrifugation, e.g., cesium chloride (CsCl) and sucrose gradients, and the like (see, e.g., Kirnbauer et al., J. Virol. (1993) 67:6929-6936), ion exchange chromatography (including anion exchange chromatography such as DMAE and TMAE), hydroxyapatitie chromatography (see WO 00/09671), hydrophobic interaction chromatography, gel filtration chromatography and other filtration methods such as nanometric filtration and ultrafiltration. Preferably at least one anion exchange step is performed during purification, and more preferably at least two anion exchange steps are used.

VLP formulations of the invention may be further processed by methods known in the art to disassemble the VLPs into smaller, protein containing moieties using a high concentration of reducing agent, followed by reassembly of the VLPs by either removal of the reducing agent or by addition of excess oxidant. The resulting reassembled VLPs may have improved homogeneity, stability and immunogenic properties. In addition, further therapeutic or prophylactic agents may be formulated into the VLPs upon reassembly. See McCarthy et al., J. Virology (1998) 72(1):32-41. See also WO 99/13056 and WO 01/42780. Reducing agents suitable for use in VLP disassembly include sulfhydryl reducing agents (such as glutathion, beta mercaptoethanol, dithiothreitol, dithioerythritol, cysteine, hydrogen sulfide and mixtures thereof) preferably contained in moderate to low ionic strength buffers. Sufficient exposure time of the VLPs to the reducing agent will be required to achieve a suitable amount of VLP disassembly.

Adjuvants may be added to the VLPs of the invention to enhance the immunogenicity of the SARS viral antigens. Antigens suitable for use with VLPs include those described, supra. For example, the VLPs of the invention may be adsorbed onto an aluminum adjuvant.

The VLPs of the invention may formulated to enhance their stability. Additional components which may enhance the stability of a VLP formulation include salts, buffers, non-ionic surfactants and other stabilizers such as polymeric polyanion stabilizers. See WO 00/45841.

The ionic strength of a solution comprising VLP particles may be maintained by the presence of salts. Almost any salt which can contribute to the control of the ionic strength may be used. Preferred salts which can be used to adjust ionic strength include physiologically acceptable salts such as NaCl, KCl, Na₂SO₄, (NH₄)₂SO₄, sodium phosphate and sodium citrate. Preferably, the salt component is present in concentrations of from about 0.10 M to 1 M. Very high concentrations are not preferred due to the practical limitations of parenteral injection of high salt concentrations. Instead, more moderate salt concentrations, such as more physiological concentrations of about 0.15M to about 0.5M with 0.15M-0.32M NaCl are preferred.

Buffers may also be used to enhance the stability of the VLP formulations of the invention. Preferably, the buffer optimizes the VLP stability while maintaining the pH range so that the vaccine formulation will not be irritating to the recipient. Buffers preferably maintain the pH of the vaccine formulation within a range of p/H 5.5-7.0, more preferably 6.0-6.5. Buffers suitable for vaccine formulations are known in the art and include, for example, histidine and imidazole. Preferably, the concentration of the buffer will range from about 2 mM to about 100 mM, more preferably 5 mM to about 20 mM. Phosphate containing buffers are generally not preferred when the VLP is adsorbed or otherwise formulated with an aluminum compound.

Non-ionic surfactants may be used to enchance the stability of the VLP formulations of the invention. Surfactants suitable for use in vaccine formulations are known in the art and include, for example, polyoxyethylene sorbital fatty acid esters (Polysorbates) such as Polysorbate 80 (e.g., TWEEN 80), Polysorbate 20 (e.g., TWEEN 20), polyoxyethylene alkyl ethers (e.g., Brij 35, Brij 58), as well as others, including Triton X-100, Triton X-114, NP-40, Span 85 and the Pluronic series of non-ionic surfactants (e.g., Pluronic 121). The surfactant is preferably present in a concentration of from about 0.0005% to about 0.5% (wt/vol).

Polymeric polyanion stabilizers may also be used to enchance the stability of the VLP formulations of the invention. Suitable polymeric polyanionic stabilizers for use in the invention comprise either a single long chain or multiple cross linked chains; either type possessing multiple negative charges along the chains when in solution. Examples of suitable polyanionic polymers include proteins, polyanions, peptides and polynucelic acids. Specific examples include carboxymethyl cellulose, heparin, polyamino acids (such as poly(Glu), poly(Asp), and Poly (Glu, Phe), oxidized glutathione, polynuceltodies, RNA, DNA and serum albumins. The concentration of the polmeric polyanion stabilizers is preferably from about 0.01% to about 0.5%, particularly about 0.05-0.1% (by weight).

G. Passive Immunization via Antibodies to the SARS Antigens of the Invention

The invention includes antibodies specific to the SARS antigens of the invention and methods of treatment or prevention of SARS virus related disease by administrating an effective amount of SARS antibodies to a mammalian subject. Antibodies specific the SARS antigens can be produced by one skilled in the art. Preferably, the antibodies are specific to the spike (S) protein of the SARS virus. Potent neutralization of the SARS coronavirus using a human monoclonal anti-spike antibody has been reported (Sui et al. (2004) PNAS USA 101:2536-2541). A IgG1 form of the monoclonal antibody showed a higher affinity (1.59 nM) than a scFv form (32.3 nM).

The antibodies of the invention are specific and selective to SARS antigens.

In one embodiment, the antibodies of the invention are generated by administering a SARS antigen to an animal. The method may also include isolating the antibodies from the animal.

The antibodies of the invention may be polyclonal or monoclonal antibody preparations, monospecific antisera, human antibodies, or may be hybrid or chimeric antibodies, such as humanized antibodies, altered antibodies (Fab′)₂ fragments, F(ab) fragments, Fv fragments, single-domain antibodies, dimeric or trimeric antibody fragments or constructs, minibodies, or functional fragments thereof which bind to the antigen in question.

Antibodies are produced using techniques well known to those of skill in the art and disclosed in, for example, U.S. Pat. Nos. 4,011,308; 4,722,890; 4,016,043; 3,876,504; 3,770,380; and 4,372,745. For example, polyclonal antibodies are generated by immunizing a suitable animal, such as a mouse, rat, rabbit, sheep, or goat, with an antigen of interest. In order to enhance immunogenicity, the antigen can be linked to a carrier prior to immunization. Such carriers are well known to those of ordinary skill in the art. Immunization is generally performed by mixing or emulsifying the antigen in saline, preferably in an adjuvant such as Freund's complete adjuvant, and injecting the mixture or emulsion parenterally (generally subcutaneously or intramuscularly). The animal is generally boosted 2-6 weeks later with one or more injections of the antigen in saline, preferably using Freund's incomplete adjuvant. Antibodies may also be generated by in vitro immunization, using methods known in the art. Polyclonal antiserum is then obtained from the immunized animal.

Monoclonal antibodies are generally prepared using the method of Kohler & Milstein (1975) Nature 256:495-497, or a modification thereof. Typically, a mouse or rat is immunized as described above. Rabbits may also be used. However, rather than bleeding the animal to extract serum, the spleen (and optionally several large lymph nodes) is removed and dissociated into single cells. If desired, the spleen cells may be screened (after removal of non-specifically adherent cells) by applying a cell suspension to a plate or well coated with the antigen. B-cells, expressing membrane-bound immunoglobulin specific for the antigen, will bind to the plate, and are not rinsed away with the rest of the suspension. Resulting B-cells, or all dissociated spleen cells, are then induced to fuse with myeloma cells to form hybridomas, and are cultured in a selective medium (e.g., hypoxanthine, aminopterin, thymidine medium, “HAT”). The resulting hybridomas are plated by limiting dilution, and are assayed for the production of antibodies which bind specifically to the immunizing antigen (and which do not bind to unrelated antigens). The selected monoclonal antibody-secreting hybridomas are then cultured either in vitro (e.g., in tissue culture bottles or hollow fiber reactors), or in vivo (e.g., as ascites in mice).

Humanized and chimeric antibodies are also useful in the invention. Hybrid (chimeric) antibody molecules are generally discussed in Winter et al. (1991) Nature 349: 293-299 and U.S. Pat. No. 4,816,567. Humanized antibody molecules are generally discussed in Riechmann et al. (1988) Nature 332:323-327; Verhoeyan et al. (1988) Science 239:1534-1536; and U.K. Patent Publication No. GB 2,276,169, published 21 Sep. 1994). One approach to engineering a humanized antibody involves cloning recombinant DNA containing the promoter, leader, and variable-region sequences from a mouse antibody gene and the constant-region exons from a human antibody gene to create a mouse-human chimera, a humanized antibody. See generally, Kuby, “Immunology, 3^{rd} Edition”, W.H. Freeman and Company, New York (1998) at page 136.

Antibody fragments which retain the ability to recognize a SARS antigen are also included within the scope of the invention. A number of antibody fragments are known in the art which comprise antigen-binding sites capable of exhibiting immunological binding properties of an intact antibody molecule. For example, functional antibody fragments can be produced by cleaving a constant region, not responsible for antigen binding, from the antibody molecule, using e.g., pepsin, to produce F(ab′)₂ fragments. These fragments will contain two antigen binding sites, but lack a portion of the constant region from each of the heavy chains. Similarly, if desired, Fab fragments, comprising a single antigen binding site, can be produced, e.g., by digestion of polyclonal or monclonal antibodies with papain. Functional fragments, includnig only the variable regions of the heavy and light chains, can also be produced, using standard techniques such as recombinant production or preferential proteolytic cleavage of immunoglobulin molecules. These fragments are known as Fᵥ. See, e.g., Inbar et al. (1972) Proc. Nat. Acad. Sci USA 69:2659-2662; Hochman et al. (1976) Biochem 15:2706-2710; and Ehrlich et al. (1980) Biochem 19:4091-4096.

A single-chain Fv (“sFv” or scFv”) polypeptide is a covalently linked V_{H}-V_{L} heterodimer which is expressed from a gene fusion including V_{H}- and V_{L}-encoding genes linked by a peptide-encoding linker. Huston et al. (1988) Proc. Nat. Acad. Sci. USA 85:5879-5883. A number of methods have been described to discern and develop chemical strucutres (linkers) for converting the naturally aggregated, but chemically separated, light and heavy polypeptide chains from an antibody V region into an sFv molecule which will fold into a three dimensional structure substantially similar to the structure of an antigen-binding site. See, e.g., U.S. Pat. Nos. 5,091,513; 5,132,405; and 4,946,778. The sFv molecules may be produced using methods described in thea rt. See, e.g., Huston et al. (1988) Proc. Nat. Acad. Sci USA 85:5879-5338; U.S. Pat. Nos. 5,091,513; 5,132,405 and 4,946,778. Design criteria include determining the appropriate length to span the distance between the C-terminus of one chain and the N-terminus of the other, wherein the linker is generally formed from small hydrophilic amino acid residues that do not coil or form secondary structures. Such methods have been described in the art. See, e.g., U.S. Pat. Nos. 5,091,513; 5,132,405 and 4,946,778. Suitable linkers generally comprise polypeptide chains of alternating sets of glycine and serine residues, and may include glutamic acid and lysine residues inserted to enhance solubility. Anti-spike scFv antibodies have been reported (Sui et al. (2004) PNAS USA 101:2536-2541).

“Mini-antibodies” or “minibodies” will also find use with the present invention. Minibodies are sFv polypeptide chains which include oligomerization domains at their C-termini, separated from the sFv by a hinge region. Pack et al., (1992) Biochem 31:1579-1584. The oligomerization domain comprises self-associating α-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al., (1992) Biochem 31:1579-1584; Cumber et al. (1992) J. Immunology 149B: 120-126.

Non-conventional means can also be used to generate and identify the antibodies of the invention. For example, a phage display library can be screened for antibodies which bind to the SARS antigens of the invention. See generally, Siegel, “Recombinant Monoclonal Antibody Technology”, Transfus. Clin. Biol. (2002) 9(1): 15-22; Sidhu, “Phage Display in Pharmaceutical Biotechnology”, Curr. Opin. Biotechnol. (2000) 11(6):610-616; Sharon, et al., “Recombinant Polyclonal Antibody Libraries”, Comb. Chem. High Throughput Screen (2000) 3(3): 185-196; and Schmitz et al., “Phage Display: A Molecular Tool for the Generation of Antibodies-Review”, Placenta, (2000) 21 SupplA: S106-12.

The antibodies of the invention may also be generated by administering the polynucleotide sequence encoding for the SARS antigen into an animal. The SARS antigen is then expressed in vivo, and antibodies specific to the SARS antigen are generated in vivo. Methods for polynucleotide delivery of the SARS antigens of the invention are discussed in section 4 below.

The antibodies of the invention are preferably specific to the SARS virus.

H. Combinations of One or More of any of the Above Approaches in a Vaccine

The compositions of the invention further comprise combinations of one or more of the compositions discussed above. For instance, the invention comprises a composition comprising an attenuated SARS virus and a subunit SARS viral antigen.

I. Combinations of SARS Antigens and Other Respiratory Virus Antigens

The invention further relates to vaccine formulations comprising one or more SARS virus antigens and one or more other respiratory virus antigens. Additional respiratory virus antigens suitable for use in the invention include antigens from influenza virus, human rhinovirus (HRV), parainfluenza virus (PIV), respiratory syncytial virus (RSV), adenovirus, metapneumovirus, and rhinovirus. The additional respiratory virus antigen could also be from a coronavirus other than the SARS coronavirus, such as the NL63 human coronavirus (van der Hoek et al. (2004) Nature Medicine 10:368-373). Preferably, the additional respiratory virus antigen is an influenza viral antigen.

The invention may also comprise one or more bacterial or viral antigens in combination with the SARS viral antigen. Antigens may be used alone or in any combination. (See, e.g., WO 02/00249 describing the use of combinations of bacterial antigens). The combinations may include multiple antigens from the same pathogen, multiple antigens from different pathogens or multiple antigens from the same and from different pathogens. Thus, bacterial, viral, and/or other antigens may be included in the same composition or may be administered to the same subject separately. It is generally preferred that combinations of antigens be used to raise an immune response be used in combinations.

Non-limiting examples of bacterial pathogens which may be used in the invention include diphtheria (See, e.g., Chapter 3 of Vaccines, 1998, eds. Plotkin & Mortimer (ISBN 0-7216-1946-0), staphylococcus (e.g., Staphylococcus aureus as described in Kuroda et al. (2001) Lancet 357:1225-1240), cholera, tuberculosis, C. tetani, also known as tetanus (See, e.g., Chapter 4 of Vaccines, 1998, eds. Plotkin & Mortimer (ISBN 0-7216-1946-0), Group A and Group B streptococcus (including Streptococcus pneumoniae, Streptococcus agalactiae and Streptococcus pyogenes as described, for example, in Watson et al. (2000) Pediatr. Infect. Dis. J. 19:331-332; Rubin et al. (2000) Pediatr Clin. North Am. 47:269-284; Jedrzejas et al. (2001) Microbiol Mol Biol Rev 65:187-207; Schuchat (1999) Lancet 353:51-56; GB patent applications 0026333.5; 0028727.6; 015640.7; Dale et al. (1999) Infect Dis Clin North Am 13:227-1243; Ferretti et al. (2001) PNAS USA 98:4658-4663), pertussis (See, e.g., Gusttafsson et al. (1996) N. Engl. J. Med. 334:349-355; Rappuoli et al. (1991) TIBTECH 9:232-238), meningitis, Moraxella catarrhalis (See, e.g., McMichael (2000) Vaccine 19 Suppl. 1:S101-107) and other pathogenic states, including, without limitation, Neisseria meningitides (A, B, C, Y), Neisseria gonorrhoeae (See, e.g., WO 99/24578; WO 99/36544; and WO 99/57280), Helicobacter pylori (e.g., CagA, VacA, NAP, HopX, HopY and/or urease as described, for example, WO 93/18150; WO 99/53310; WO 98/04702) and Haemophilus influenza. Hemophilus influenza type B (HIB) (See, e.g., Costantino et al. (1999) Vaccine 17:1251-1263), Porphyromonas gingivalis (Ross et al. (2001) Vaccine 19:4135-4132) and combinations thereof.

Non-limiting examples of viral pathogens which may be used in the invention include meningitis, rhinovirus, influenza (Kawaoka et al., Virology (1990) 179:759-767; Webster et al., “Antigenic variation among type A influenza viruses,” p. 127-168. In: P. Palese and D. W. Kingsbury (ed.), Genetics of influenza viruses. Springer-Verlag, New York), respiratory syncytial virus (RSV), parainfluenza virus (PIV), rotavirus (e.g., VP1, VP2, VP3, VP4, VP6, VP7, NSP1, NSP2, NSP3, NSP4 or NSP5 and other rotavirus antigens, for example as described in WO 00/26380) and the like. Antigens derived from other viruses will also find use in the present invention, such as without limitation, proteins from members of the families Picomaviridae (e.g., polioviruses, etc. as described, for example, in Sutter et al. (2000) Pediatr Clin North Am 47:287-308; Zimmerman & Spann (1999) Am Fam Physician 59:113-118; 125-126); Caliciviridae; Togaviridae (e.g., rubella virus, etc.); Flaviviridae, including the genera flavivirus (e.g., yellow fever virus, Japanese encephalitis virus, serotypes of Dengue virus, tick borne encephalitis virus, West Nile virus, St. Louis encephalitis virus); pestivirus (e.g., classical porcine fever virus, bovine viral diarrhea virus, border disease virus); and hepacivirus (e.g., hepatitis A, B and C as described, for example, in U.S. Pat. Nos. 4,702,909; 5,011,915; 5,698,390; 6,027,729; and 6,297,048); Parvovirus (e.g., parvovirus B19); Coronaviridae; Reoviridae; Bimaviridae; Rhabodoviridae (e.g., rabies virus, etc. as described for example in Dressen et al. (1997) Vaccine 15 Suppl:s2-6; MMWR Morb Mortal Wkly Rep. Jan. 16, 1998; 47(1):12, 19); Filoviridae; Paramyxoviridae (e.g., mumps virus, measles virus, respiratory syncytial virus, etc. as described in Chapters 9 to 11 of Vaccines, 1998, eds. Plotkin & Mortimer (ISBN 0-7216-1946-0); Orthomyxoviridae (e.g., influenza virus types A, B and C, etc. as described in Chapter 19 of Vaccines, 1998, eds. Plotkin & Mortimer (ISBN 0-7216-1946-0),); Bunyaviridae; Arenaviridae; Retroviradae (e.g., HTLV-1; HTLV-11; HIV-1 (also known as HTLV-III, LAV, ARV, HTI,R, etc.)), including but not limited to antigens from the isolates HIVI11b, HIVSF2, HIVLAV, HIVI-AL, I-IIVMN, SF162); HIV-I CM235, HIV-I US4; HIV-2; simian immunodeficiency virus (SIV) among others. Additionally, antigens may also be derived from human papilloma virus (HPV) and the tick-borne encephalitis viruses. See, e.g. Virology, 3^{rd} Edition (W. K. Joklik ed. 1988); Fundamental Virology, 2nd Edition (B. N. Fields and D. M. Knipe, eds, 1991), for a description of these and other viruses.

Proteins may also be derived from the herpesvirus family, including proteins derived from herpes simplex virus (HSV) types 1 and 2, such as HSV-1 and HSV-2 glycoproteins gB, gD and gH; antigens derived from varicella zoster virus (VZV), Epstein-Barr virus (EBV) and cytomegalovirus (CMV) including CMV gB and gH (See, U.S. Pat. No. 4,689,225 and PCT Publication WO 89/07143); and antigens derived from other human herpesviruses such as HHV6 and HHV7. (See, e.g. Chee et al., Cytomegaloviruses (J. K. McDougall, ed., Springer-Verlag 1990) pp. 125-169, for a review of the protein coding content of cytomegalovirus; McGeoch et al., J. Gen. Virol. (1988) 69:1531-1574, for a discussion of the various HSV-1 encoded proteins; U.S. Pat. No. 5,171,568 for a discussion of HSV-1 and HSV-2 gB and gD proteins and the genes encoding therefor; Baer et al., Nature (1984) 310:207-211, for the identification of protein coding sequences in an EBV genome; and Davison and Scott, J. Gen. Virol. (1986) 67:1759-1816, for a review of VZV). Herpes simplex virus (HSV) rgD2 is a recombinant protein produced in genetically engineered Chinese hamster ovary cells. This protein has the normal anchor region truncated, resulting in a glycosylated protein secreted into tissue culture medium. The gD2 can be purified in the CHO medium to greater than 90% purity. Human immunodeficiency virus (HIV) env-2-3 is a recombinant form of the HIV enveloped protein produced in genetically engineered Saccharomyces cerevisae. This protein represents the entire protein region of HIV gp120 but is non-glycosylated and denatured as purified from the yeast. HIV gp120 is a fully glycosylated, secreted form of gp120 produced in CHO cells in a fashion similar to the gD2 above. Additional HSV antigens suitable for use in immunogenic compositions are described in PCT Publications W0 85/04587 and W0 88/02634, the disclosures of which are incorporated herein by reference in their entirety. Mixtures of gB and gD antigens, which are truncated surface antigens lacking the anchor regions, are particularly preferred.

Antigens from the hepatitis family of viruses, including hepatitis A virus (HAV) (See, e.g., Bell et al. (2000) Pediatr Infect Dis. J. 19:1187-1188; Iwarson (1995) APMIS 103:321-326), hepatitis B virus (HBV) (See, e.g., Gerlich et al. (1990) Vaccine 8 Suppl:S63-68 & 79-80), hepatitis C virus (HCV) (See, e.g., PCT/US88/04125, published European application number 318216), the delta hepatitis virus (HDV), hepatitis E virus (HEV) and hepatitis G virus (HGV), can also be conveniently used in the techniques described herein. By way of example, the viral genomic sequence of HCV is known, as are methods for obtaining the sequence. See, e.g., International Publication Nos. WO 89/04669; WO 90/11089; and WO 90/14436. Also included in the invention are molecular variants of such polypeptides, for example as described in PCT/US99/31245; PCT/US99/31273 and PCT/US99/31272. The HCV genome encodes several viral proteins, including E1 (also known as E) and E2 (also known as E2/NSI) and an N-terminal nucleocapsid protein (termed “core”) (see, Houghton et al., Hepatology (1991) 14:381-388, for a discussion of HCV proteins, including E1 and E2). Similarly, the sequence for the δ-antigen from HDV is known (see, e.g., U.S. Pat. No. 5,378,814) and this antigen can also be conveniently used in the present composition and methods. Additionally, antigens derived from HBV, such as the core antigen, the surface antigen, SAg, as well as the presurface sequences, pre-S1 and pre-S2 (formerly called pre-S), as well as combinations of the above, such as SAg/pre-S1, SAg/pre-S2, SAg/pre-S1/pre-S2, and pre-S1/pre-S2, will find use herein. See, e.g., “HBV Vaccines—from the laboratory to license: a case study” in Mackett, M. and Williamson, J. D., Human Vaccines and Vaccination, pp. 159-176, for a discussion of HBV structure; and U.S. Pat. Nos. 4,722,840, 5,098,704, 5,324,513, incorporated herein by reference in their entireties; Beames et al., J. Virol. (1995) 69:6833-6838, Birnbaum et al., J. Virol. (1990) 64:3319-3330; and Zhou et al., J. Virol. (1991) 65:5457-5464. Each of these proteins, as well as antigenic fragments thereof, will find use in the present composition and methods.

Influenza virus is another example of a virus for which the present invention will be particularly useful. Specifically, the envelope glycoproteins HA and NA of influenza A are of particular interest for generating an immune response. Numerous HA subtypes of influenza A have been identified (Kawaoka et al., Virology (1990) 179:759-767; Webster et al., “Antigenic variation among type A influenza viruses,” p. 127-168. In: P. Palese and D. W. Kingsbury (ed.), Genetics of influenza viruses. Springer-Verlag, New York). Thus, proteins derived from any of these isolates can also be used in the compositions and methods described herein.

Non-limiting examples of parasitic antigens include those derived from organisms causing malaria and Lyme disease.

The methods of the invention comprise administering an immunogenic composition comprising a SARS viral antigen (including one or more of an inactivated SARS virus, an attenuated SARS virus, a split SARS virus preparation or a recombinant or purified subunit formulation of one or more SARS viral antigens) to an animal. The immunogenic compositions used in the invention can comprise an immunologically effective amount of the SARS viral antigen. An “immunologically effective amount” is an amount sufficient to allow the mammal to raise an immune response to the SARS antigen.

The immune response preferably involves the production of antibodies specific to the SARS antigen. The amount of antibodies produced will vary depending on several factors including the animal used, the presence of an adjuvant, etc.

The immunogenic compositions of the invention may further comprise one or more adjuvants.

The immunogenic compositions of the invention may be administered mucosally. Suitable routes of mucosal administration include oral, intranasal, intragastric, pulmonary, intestinal, rectal, ocular and vaginal routes. The immunogenic composition may be adapted for mucosal administration. For instance, where the composition is for oral administration, it may be in the form of tablets or capsules, optionally enteric-coated, liquid, transgenic plants, etc. Where the composition is for intranasal administration, it may be in the form of a nasal spray, nasal drops, gel or powder.

The immunogenic compositions of the invention may be administered parenterally. Suitable routes of parenteral administration include intramuscular (IM), subcutaneous, intravenous, intraperitoneal, intradermal, transcutaneous, and transdermal (see e.g., International patent application WO 98/20734) routes, as well as delivery to the interstitial space of a tissue. The immunogenic composition may be adapted for parenteral administration, for instance in the form of an injectable that may be sterile and pyrogen free.

Vaccines of the invention may be administered in conjunction with other immunoregulatory agents. In particular, compositions will usually include an adjuvant. Preferred further adjuvants include, but are not limited to, one or more of the following set forth below:

A. Mineral Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphoshpates, orthophosphates), sulphates, etc. (e.g. see chapters 8 & 9 of Vaccine design: the subunit and adjuvant approach (1995) Powell & Newman. ISBN 0-306-44867-X.), or mixtures of different mineral compounds, with the compounds taking any suitable form (e.g. gel, crystalline, amorphous, etc.), and with adsorption being preferred. The mineral containing compositions may also be formulated as a particle of metal salt. See WO00/23105.

B. Oil-Emulsions

Oil-emulsion compositions suitable for use as adjuvants in the invention include squalene-water emulsions, such as MF59 (5% Squalene, 0.5% Tween 80, and 0.5% Span 85, formulated into submicron particles using a microfluidizer). See WO90/14837. See also, Frey et al., “Comparison of the safety, tolerability, and immunogenicity of a MF59-adjuvanted influenza vaccine and a non-adjuvanted influenza vaccine in non-elderly adults”, Vaccine (2003) 21:4234-4237.

Particularly preferred adjuvants for use in the compositions are submicron oil-inwater emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween 80™ (polyoxyelthylenesorbitan monooleate), and/or 0.25-1.0% Span 85™ (sorbitan trioleate), and, optionally, N-acetylmuramyl-L-alanyl-D-isogluatminyl-L-alanine-2-(1′-2′-dipalmitoyl-sn-glycero-3-huydroxyphosphophoryloxy)-ethylamine (MTP-PE), for example, the submicron oil-in-water emulsion known as “MF59” (International Publication No. WO 90/14837; U.S. Pat. Nos. 6,299,884 and 6,451,325, incorporated herein by reference in their entireties; and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g., 4.3%), 0.25-0.5% w/v Tween 80™, and 0.5% w/v Span 85™ and optionally contains various amounts of MTP-PE, formulated into submicron particles using a microfluidizer such as Model 110Y microfluidizer (Microfluidics, Newton, Mass.). For example, MTP-PE may be present in an amount of about 0-500 μg/dose, more preferably 0-250 μg/dose and most preferably, 0-100 μg/dose. As used herein, the term “MF59-0” refers to the above submicron oil-in-water emulsion lacking MTP-PE, while the term MF59-MTP denotes a formulation that contains MTP-PE. For instance, “MF59-100” contains 100 μg MTP-PE per dose, and so on. MF69, another submicron oil-in-water emulsion for use herein, contains 4.3% w/v squalene, 0.25% w/v Tween 80™, and 0.75% w/v Span 85™ and optionally MTP-PE. Yet another submicron oil-in-water emulsion is MF75, also known as SAF, containing 10% squalene, 0.4% Tween 80™, 5% pluronic-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO 90114837 and U.S. Pat. Nos. 6,299,884 and 6,451,325, incorporated herein by reference in their entireties.

Complete Freund's adjuvant (CFA) and incomplete Freund's adjuvant (IFA) may also be used as adjuvants in the invention.

C. Saponin Formulations

Saponin formulations, may also be used as adjuvants in the invention. Saponins are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs.

Saponin compositions have been purified using High Performance Thin Layer Chromatography (HP-LC) and Reversed Phase High Performance Liquid Chromatography (RP-HPLC). Specific purified fractions using these techniques have been identified, including QS7, QS17, QS18, QS21, QH-A, QH-B and QH-C. Preferably, the saponin is QS21. A method of production of QS21 is disclosed in U.S. Pat. No. 5,057,540. Saponin formulations may also comprise a sterol, such as cholesterol (see WO 96/33739).

Combinations of saponins and cholesterols can be used to form unique particles called Immunostimulating Complexs (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP 0 109 942, WO 96/11711 and WO 96/33739. Optionally, the ISCOMS may be devoid of additional detergent. See WO00/07621.

A review of the development of saponin based adjuvants can be found at Barr, et al., “ISCOMs and other saponin based adjuvants”, Advanced Drug Delivery Reviews (1998) 32:247-271. See also Sjolander, et al., “Uptake and adjuvant activity of orally delivered saponin and ISCOM vaccines”, Advanced Drug Delivery Reviews (1998) 32:321-338.

D. Bacterial or Microbial Derivatives

Adjuvants suitable for use in the invention include bacterial or microbial derivatives such as:

(1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives e.g. RC-529. See Johnson et al. (1999) Bioorg Med Chem Lett 9:2273-2278.

(2) Lipid A Derivatives

Lipid A derivatives include derivatives of lipid A from Escherichia coli such as OM-174. OM-174 is described for example in Meraldi et al., “OM-174, a New Adjuvant with a Potential for Human Use, Induces a Protective Response with Administered with the Synthetic C-Terminal Fragment 242-310 from the circumsporozoite protein of Plasmodium berghei”, Vaccine (2003) 21:2485-2491; and Pajak, et al., “The Adjuvant OM-174 induces both the migration and maturation of murine dendritic cells in vivo”, Vaccine (2003) 21:836-842.

(3) Immunostimulatory Oligonucleotides

Immunostimulatory oligonucleotides suitable for use as adjuvants in the invention include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpG's can include nucleotide modifications/analogs such as phosphorothioate modifications and can be double-stranded or single-stranded. Optionally, the guanosine may be replaced with an analog such as 2′-deoxy-7-deazaguanosine. See Kandimalla, et al., “Divergent synthetic nucleotide motif recognition pattern: design and development of potent immunomodulatory oligodeoxyribonucleotide agents with distinct cytokine induction profiles”, Nucleic Acids Research (2003) 31(9): 2393-2400; WO 02/26757 and WO 99/62923 for examples of possible analog substitutions. The adjuvant effect of CpG oligonucleotides is further discussed in Krieg, “CpG motifs: the active ingredient in bacterial extracts?”, Nature Medicine (2003) 9(7): 831-835; McCluskie, et al., “Parenteral and mucosal prime-boost immunization strategies in mice with hepatitis B surface antigen and CpG DNA”, FEMS Immunology and Medical Microbiology (2002) 32:179-185; WO 98/40100; U.S. Pat. No. 6,207,646; U.S. Pat. No. 6,239,116 and U.S. Pat. No. 6,429,199.

The CpG sequence may be directed to TLR9, such as the motif GTCGTT or TTCGTT. See Kandimalla, et al., “Toll-like receptor 9: modulation of recognition and cytokine induction by novel synthetic CpG DNAs”, Biochemical Society Transactions (2003) 31 (part 3): 654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell, et al., “CpG-A-Induced Monocyte IFN-gamma-Inducible Protein-10 Production is Regulated by Plasmacytoid Dendritic Cell Derived IFN-alpha”, J. Immunol. (2003) 170(8):4061-4068; Krieg, “From A to Z on CpG”, TRENDS in Immunology (2002) 23(2): 64-65 and WO 01/95935. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, Kandimalla, et al., “Secondary structures in CpG oligonucleotides affect immunostimulatory activity”, BBRC (2003) 306:948-953; Kandimalla, et al., “Toll-like receptor 9: modulation of recognition and cytokine induction by novel synthetic GpG DNAs”, Biochemical Society Transactions (2003) 31(part 3):664-658; Bhagat et al., “CpG penta- and hexadeoxyribonucleotides as potent immunomodulatory agents” BBRC (2003) 300:853-861 and WO 03/035836.

(4) ADP-Ribosylating Toxins and Detoxified Derivatives Thereof.

Bacterial ADP-ribosylating toxins and detoxified derivatives thereof may be used as adjuvants in the invention. Preferably, the protein is derived from E. coli (i.e., E. coli heat labile enterotoxin “LT), cholera (“CT”), or pertussis (“PT”). The use of detoxified ADP-ribosylating toxins as mucosal adjuvants is described in WO 95/17211 and as parenteral adjuvants in WO 98/42375. Preferably, the adjuvant is a detoxified LT mutant such as LT-K63, LT-R72, and LTR192G. The use of ADP-ribosylating toxins and detoxified derivaties thereof, particularly LT-K63 and LT-R72, as adjuvants can be found in the following references, each of which is specifically incorporated by reference herein in their entirety: Beignon, et al., “The LTR72 Mutant of Heat-Labile Enterotoxin of Escherichia coli Enahnces the Ability of Peptide Antigens to Elicit CD4+ T Cells and Secrete Gamma Interferon after Coapplication onto Bare Skin”, Infection and Immunity (2002) 70(6):3012-3019; Pizza, et al., “Mucosal vaccines: non-toxic derivatives of LT and CT as mucosal adjuvants”, Vaccine (2001) 19:2534-2541; Pizza, et al., “LTK63 and LTR72, two mucosal adjuvants ready for clinical trials” Int. J. Med. Microbiol (2000) 290(4-5):455-461; Scharton-Kersten et al., “Transcutaneous Immunization with Bacterial ADP-Ribosylating Exotoxins, Subunits and Unrelated Adjuvants”, Infection and Immunity (2000) 68(9):5306-5313; Ryan et al., “Mutants of Escherichia coli Heat-Labile Toxin Act as Effective Mucosal Adjuvants for Nasal Delivery of an Acellular Pertussis Vaccine: Differential Effects of the Nontoxic AB Complex and Enzyme Activity on Th1 and Th2 Cells” Infection and Immunity (1999) 67(12):6270-6280; Partidos et al., “Heat-labile enterotoxin of Escherichia coli and its site-directed mutant LTK63 enhance the proliferative and cytotoxic T-cell responses to intranasally co-immunized synthetic peptides”, Immunol. Lett. (1999) 67(3):209-216; Peppoloni et al., “Mutants of the Escherichia coli heat-labile enterotoxin as safe and strong adjuvants for intranasal delivery of vaccines”, Vaccines (2003) 2(2):285-293; and Pine et al., (2002) “Intranasal immunization with influenza vaccine and a detoxified mutant of heat labile enterotoxin from Escherichia coli (LTK63)” J. Control Release (2002) 85(1-3):263-270. Numerical reference for amino acid substitutions is preferably based on the alignments of the A and B subunits of ADP-ribosylating toxins set forth in Domenighini et al., Mol. Microbiol (1995) 15(6): 1165-1167, specifically incorporated herein by reference in its entirety.

E. Human Immunomodulators

Human immunomodulators suitable for use as adjuvants in the invention include cytokines, such as interleukins (e.g. IL-1, IL-2, IL-4, IL-5, IL-6, IL-7, IL-12, etc.), interferons (e.g. interferon-γ), macrophage colony stimulating factor, and tumor necrosis factor.

F. Bioadhesives and Mucoadhesives

Bioadhesives and mucoadhesives may also be used as adjuvants in the invention. Suitable bioadhesives include esterified hyaluronic acid microspheres (Singh et al. (2001) J. Cont. Rele. 70:267-276) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention. E.g., WO99/27960.

G. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

H. Liposomes

Examples of liposome formulations suitable for use as adjuvants are described in U.S. Pat. No. 6,090,406, U.S. Pat. No. 5,916,588, and EP 0 626 169.

I. Polyoxyethylene Ether and Polyoxyethylene Ester Formulations

Adjuvants suitable for use in the invention include polyoxyethylene ethers and polyoxyethylene esters. WO99/52549. Such formulations further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152).

Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

J. Polyphosphazene (PCPP)

PCPP formulations are described, for example, in Andrianov et al., “Preparation of hydrogel microspheres by coacervation of aqueous polyphophazene solutions”, Biomaterials (1998) 19(1-3):109-115 and Payne et al., “Protein Release from Polyphosphazene Matrices”, Adv. Drug. Delivery Review (1998) 31(3):185-196.

K. Muramyl peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylaamine MTP-PE).

L. Imidazoquinolone Compounds.

Examples of imidazoquinolone compounds suitable for use adjuvants in the invention include Imiquamod and its homologues, described further in Stanley, “Imiquimod and the imidazoquinolones: mechanism of action and therapeutic potential” Clin Exp Dermatol (2002) 27(7):571-577 and Jones, “Resiquimod 3M”, Curr Opin Investig Drugs (2003) 4(2):214-218

M. Virosomes and Virus Like Particles (VLPs)

Virosomes and Virus Like Particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO 03/024480, WO 03/024481, and Niukura et al., “Chimeric Recombinant Hepatitis E Virus-Like Particles as an Oral Vaccine Vehicle Presenting Foreign Epitopes”, Virology (2002) 293:273-280; Lenz et al., “Papillomarivurs-Like Particles Induce Acute Activation of Dendritic Cells”, Journal of Immunology (2001) 5246-5355; Pinto, et al., “Cellular Immune Responses to Human Papillomavirus (HPV)-16 L1 Healthy Volunteers Immunized with Recombinant HPV-16 L1 Virus-Like Particles”, Journal of Infectious Diseases (2003) 188:327-338; and Gerber et al., “Human Papillomavrisu Virus-Like Particles Are Efficient Oral immunogens when Coadministered with Escherichia coli Heat-Labile Entertoxin Mutant R192G or CpG”, Journal of Virology (2001) 75(10):4752-4760. Virosomes are discussed further in, for example, Gluck et al., “New Technology Platforms in the Development of Vaccines for the Future”, Vaccine (2002) 20:B10-B16.

The invention may also comprise combinations of aspects of one or more of the adjuvants identified above. For example, the following adjuvant compositions may be used in the invention:

(1) a saponin and an oil-in-water emulsion (WO99/11241);

(2) a saponin (e.g., QS21)+a non-toxic LPS derivative (e.g., 3dMPL) (see WO 94/00153);

(3) a saponin (e.g., QS21)+a non-toxic LPS derivative (e.g., 3dMPL)+a cholesterol;

(4) a saponin (e.g. QS21)+3dMPL+IL-12 (optionally +a sterol) (WO98/57659);

(5) combinations of 3dMPL with, for example, QS21 and/or oil-in-water emulsions (See European patent applications 0835318, 0735898 and 0761231);

(6) SAF, containing 10% Squalane, 0.4% Tween 80, 5% pluronic-block polymer L121, and thr-MDP, either microfluidized into a submicron emulsion or vortexed to generate a larger particle size emulsion.

(7) Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and

(8) one or more mineral salts (such as an aluminum salt)+a non-toxic derivative of LPS (such as 3dPML).

Aluminium salts and MF59 are preferred adjuvants for parenteral immunisation. Mutant bacterial toxins are preferred mucosal adjuvants.

As mentioned above, adjuvants suitable for use in the invention may also include one or more of the following:

E. coli heat-labile enterotoxin (“LT”), or detoxified mutants thereof, such as the K63 or R72 mutants;

cholera toxin (“CT”), or detoxified mutants thereof;

microparticles (i.e., a particle of ˜100 nm to ˜150 μm in diameter, more preferably ˜200 nm to ˜30 μm in diameter, and most preferably 500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone etc.);

a polyoxyethylene ether or a polyoxyethylene ester (see International patent application WO 99/52549);

a polyoxyethylene sorbitan ester surfactant in combination with an octoxynol (see International patent application WO 01/21207) or a polyoxyethylene alkyl ether or ester surfactant in combination with at least one additional non-ionic surfactant such as an octoxynol (see International patent application WO 01/21152);

chitosan (e.g. International patent application WO 99/27960)

an immunostimulatory oligonucleotide (e.g. a CpG oligonucleotide) and a saponin (see International patent application WO 00/62800)

immunostimulatory double stranded RNA.

aluminum compounds (e.g. aluminum hydroxide, aluminum phosphate, aluminum hydroxyphosphate, oxyhydroxide, orthophosphate, sulfate etc. (e.g. see chapters 8 & 9 of Vaccine design: the subunit and adjuvant aproach, eds. Powell & Newman, Plenum Press 1995 (ISBN 0-306-44867-X) (hereinafter “Vaccine design”), or mixtures of different aluminum compounds, with the compounds taking any suitable form (e.g. gel, crystalline, amorphous etc.), and with adsorption being preferred;

MF59 (5% Squalene, 0.5% Tween 80, and 0.5% Span 85, formulated into submicron particles using a microfluidizer) (see Chapter 10 of Vaccine design; see also International patent application WO 90/14837);

liposomes (see Chapters 13 and 14 of Vaccine design);

ISCOMs (see Chapter 23 of Vaccine design);

SAF, containing 10% Squalane, 0.4% Tween 80, 5% pluronic-block polymer L121, and thr-MDP, either microfluidized into a submicron emulsion or vortexed to generate a larger particle size emulsion (see Chapter 12 of Vaccine design);

Ribi™ adjuvant system (RAS), (Ribi Immunochem) containing 2% Squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™);

saponin adjuvants, such as QuilA or QS21 (see Chapter 22 of Vaccine design), also known as Stimulon™;

ISCOMs, which may be devoid of additional detergent (WO 00/07621);

complete Freund's adjuvant (CFA) and incomplete Freund's adjuvant (IFA);

cytokines, such as interleukins (e.g. IL-1, IL-2, IL-4, IL-5, IL-6, IL-7, IL-12, etc.), interferons (e.g. interferon-γ), macrophage colony stimulating factor, tumor necrosis factor, etc. (see Chapters 27 & 28 of Vaccine design);

monophosphoryl lipid A (MPL) or 3-O-deacylated MPL (3dMPL) (e.g. chapter 21 of Vaccine design);

combinations of 3dMPL with, for example, QS21 and/or oil-in-water emulsions (European patent applications 0835318, 0735898 and 0761231);

oligonucleotides comprising CpG motifs (see Krieg (2000) Vaccine, 19:618-622; Krieg (2001) Curr. Opin. Mol. Ther., 2001, 3:15-24; WO 96/02555, WO 98/16247, WO 98/18810, WO 98/40100, WO 98/55495, WO 98/37919 and WO 98/52581, etc.) i.e. containing at least one CG dinucleotide,

a polyoxyethylene ether or a polyoxyethylene ester (International patent application WO99/52549);

a polyoxyethylene sorbitan ester surfactant in combination with an octoxynol (International patent application WO 01/21207) or a polyoxyethylene alkyl ether or ester surfactant in combination with at least one additional non-ionic surfactant such as an octoxynol (WO 01/21152);

an immunostimulatory oligonucleotide (e.g. a CpG oligonucleotide) and a saponin (WO00/62800);

an immunostimulant and a particle of metal salt (International patent application WO00/23105);

a saponin and an oil-in-water emulsion (WO 99/11241);

a saponin (e.g. QS21)+3dMPL+IL-12 (optionally+a sterol) (WO 98/57659).

Other adjuvants suitable for mucosal or parenteral administration are also available (e.g. see chapter 7 of Vaccine design: the subunit and adjuvant aproach, eds. Powell & Newman, Plenum Press 1995 (ISBN 0-306-44867-X).

Mutants of LT are preferred mucosal adjuvants, in particular the “K63” and “R72” mutants (e.g. see International patent application WO 98/18928), as these result in an enhanced immune response.

Microparticles are also preferred mucosal adjuvants. These are preferably derived from a poly(a-hydroxy acid), in particular, from a poly(lactide) (“PLA”), a copolymer of D,L-lactide and glycolide or glycolic acid, such as a poly(D,L-lactide-co-glycolide) (“PLG” or “PLGA”), or a copolymer of D,L-lactide and caprolactone. The microparticles may be derived from any of various polymeric starting materials which have a variety of molecular weights and, in the case of the copolymers such as PLG, a variety of lactide:glycolide ratios, the selection of which will be largely a matter of choice, depending in part on the coadministered antigen.

The SARS virus (inactivated or attenuated), viral antigens, antibodies or adjuvants of the invention may be entrapped within the microparticles, or may be adsorbed to them. Entrapment within PLG microparticles is preferred. PLG microparticles are discussed in further detail in Morris et al., (1994), Vaccine, 12:5-11, in chapter 13 of Mucosal Vaccines, eds. Kiyono et al., Academic Press 1996 (ISBN 012410587), and in chapters 16 & 18 of Vaccine design: the subunit and adjuvant aproach, eds. Powell & Newman, Plenum Press 1995 (ISBN 0-306-44867-X).

LT mutants may advantageously be used in combination with microparticle-entrapped antigen, resulting in significantly enhanced immune responses.

Aluminium compounds and MF59 are preferred adjuvants for parenteral use.

The composition may include an antibiotic.

The immunogenic compositions of the invention may be administered in a single dose, or as part of an administration regime. The regime may include priming and boosting doses, which may be administered mucosally, parenterally, or various combinations thereof.

The methods of the invention further comprise treating or preventing a SARS virus-related disease by administering to an animal a composition comprising an effective amount of the antibodies of the invention. An “effective amount” of the antibodies of the invention is an amount sufficient to provide passive immunization protection or treatment to the animal. Preferably, the antibodies of the invention are specific to the SARS viral antigen.

Methods of treatment may combine both immunogenic compositions and antibody compositions. Accordingly the invention comprises a method for treating or preventing a SARS virus-related disease comprising administering an immunogenic composition comprising an immunologically effective amount of a SARS viral antigen and administering an effective amount of antibodies specific to SARS viral antigen. The immunogenic composition and the antibodies may be administered together or separately. The invention further comprises a composition comprising an immunogenic composition comprising an immunologically effective amount of a SARS viral antigen and further comprising an effective amount of antibodies specific to a SARS viral antigen.

The SARS viral antigens and antibodies of the invention may also be administered in polynucleotide form. The SARS viral antigens and/or antibody proteins are then expressed in vivo.

The SARS viral antigens and the antibodies of the invention can also be delivered using one or more gene vectors, administered via nucleic acid immunization or the like using standard gene delivery protocols. Methods for gene delivery are known in the art. See, e.g., U.S. Pat. Nos. 5,399,346, 5,580,859, 5,589,466. The constructs can be delivered (e.g., injected) either subcutaneously, epidermally, intradermally, intramuscularly, intravenous, mucosally (such as nasally, rectally and vaginally), intraperitoneally, orally or combinations thereof. Intramuscular injection of 25 μg plasmid DNA encoding spike antigens, in 200 μl PBS pH 7.4, at weeks 0, 3 and 6, has been described for mice by Yang et al. (2004) Nature 428:561-564.

An exemplary replication-deficient gene delivery vehicle that may be used in the practice of the present invention is any of the alphavirus vectors, described in, for example, U.S. Pat. Nos. 6,342,372; 6,329,201 and International Publication WO 01/92552.

A number of viral based systems have been developed for gene transfer into mammalian cells. For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller & Rosman, BioTechniques (1989) 7:980-990; Miller, A. D., Human Gene Therapy (1990) 1:5-14; Scarpa et al., Virology (1991) 180:849-852; Burns et al., Proc. Natl. Acad. Sci. USA (1993) 90:8033-8037; and Boris-Lawrie & Temin, Cur. Opin. Genet. Develop. (1993) 3:102-109.

A number of adenovirus vectors have also been described. Unlike retroviruses which integrate into the host genome, adenoviruses persist extrachromosomally thus minimizing the risks associated with insertional mutagenesis (Haj-Ahmad and Graham, J. Virol. (1986) 57:267-274; Bett et al., J. Virol. (1993) 67:5911-5921; Mittereder et al., Human Gene Therapy (1994) 5:717-729; Seth et al., J. Virol. (1994) 68:933-940; Barr et al., Gene Therapy (1994) 1:51-58; Berkner, K. L. BioTechniques (1988) 6:616-629; and Rich et al., Human Gene Therapy (1993) 4:461-476). Adenoviral delivery of codon-optimsed versions of the genes encoding SARS coronavirus structural antigens spike S1, membrane protein and nucleocapsid protein has been investigated in rhesus macaques and found to invoke a strong neutralizing antibody response (Gao et al. (2003) Lancet 362(9399):1895-1896).

Additionally, various adeno-associated virus (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor Laboratory Press); Carter, B. J. Current Opinion in Biotechnology (1992) 3:533-539; Muzyczka, N. Current Topics in Microbiol. and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering polynucleotides, mucosally and otherwise, is the enterically administered recombinant poxvirus vaccines described by Small, Jr., P. A., et al. (U.S. Pat. No. 5,676,950, issued Oct. 14, 1997, herein incorporated by reference) as well as the vaccinia virus and avian poxviruses. By way of example, vaccinia virus recombinants expressing the genes can be constructed as follows. The DNA encoding the SARS antigen or antibody or antibody coding sequence is first inserted into an appropriate vector so that it is adjacent to a vaccinia promoter and flanking vaccinia DNA sequences, such as the sequence encoding thymidine kinase (TK). This vector is then used to transfect cells that are simultaneously infected with vaccinia. Homologous recombination serves to insert the vaccinia promoter plus the gene encoding the coding sequences of interest into the viral genome. The resulting TK⁻ recombinant can be selected by culturing the cells in the presence of 5-bromodeoxyuridine and picking viral plaques resistant thereto.

Alternatively, avipoxviruses, such as the fowlpox and canarypox viruses, can also be used to deliver genes encoding the SARS viral antigens or antibodies of the invention. Recombinant avipox viruses, expressing immunogens from mammalian pathogens, are known to confer protective immunity when administered to non-avian species. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545. Picornavirus-derived vectors can also be used. (See, e.g., U.S. Pat. Nos. 5,614,413 and 6,063,384).

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

A vaccinia based infection/transfection system can be conveniently used to provide for inducible, transient expression of the coding sequences of interest (for example, a SARS viral antigen or antibody expression cassette) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the polynucleotide of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation products. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

As an alternative approach to infection with vaccinia or avipox virus recombinants, or to the delivery of genes using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase that in turn will transcribe more template. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

The immunogenic compositions of the invention may further comprise diluents, such as water, saline, glycerol, ethanol, etc. Additionally, auxiliary substances, such as wetting or emulsifying agents, pH buffering substances, and the like may be included in the immunogenic composition.

The immunogenic compositions used in the invention can be administered to an animal. Animals suitable for use in the methods of the invention include humans and other primates, including non-human primates such as chimpanzees, and other apes and monkey species; farm animals such as cattle, sheep, pigs, goats and horses, domestic animals such as dogs and cats; laboratory animals including rodents such as mice, rats and guinea pigs; birds, including domestic, wild and game birds such as chickens, turkeys and other gallinaceous birds, ducks, geese and the like. Animals suitable for use in the invention can be of any age, including both adult and newborn. Transgenic animals can also be used in the invention.

The immunogenic compositions of the invention can be-used to treat or prevent SARS virus-related diseases.

The compositions of the invention are preferably pharmaceutically acceptable and pharmacologically acceptable. In particularly, the compositions are preferably not biologically or otherwise undesirable, i.e., the material may be administered to an individual in a formulation or composition without causing any undesirable biological effects or interacting in a deleterious manner with any of the components of the composition in which it is contained.

Pharmaceutically acceptable salts can also be used in compositions of the invention, for example, mineral salts such as hydrochlorides, hydrobromides, phosphates, or sulfates, as well as salts of organic acids such as acetates, proprionates, malonates, or benzoates. Especially useful protein substrates are serum albumins, keyhole limpet hemocyanin, immunoglobulin molecules, thyroglobulin, ovalbumin, tetanus toxoid, and other proteins well known to those of skill in the art. Compositions of the invention can also contain liquids or excipients, such as water, saline, glycerol, dextrose, ethanol, or the like, singly or in combination, as well as substances such as wetting agents, emulsifying agents, or pH buffering agents. Liposomes can also be used as a carrier for a composition of the invention.

SARS specific reagents and analytical assays may be used in the manufacture and testing of the vaccines of the invention. Such analytical assays include, for example: 1) virus titration and plaque assays for quantitation of infectious virus particles, 2) a neutralization assay with constant virus and varying serum dilutions, 3) a two step RT-PCR system (Light Cycler-Roche) for detection of negative strand viral RNA, with the target sequence located within the N gene, providing highest possible sensitivity, and 4) ELISA and western blot assays for detection and qualification of viral proteins.

In addition, rabbit polyclonal antiserum has been generated to obtain antibody reagents (and demonstrate induction of neutralizing antibodies) against the SARS-CoV. A sample protocol for generating such reagents is set forth below. The virus is first cultivated in suitable cell culture, such as Vero cells, and pelleted through a 20% sucrose (w/v) cushion. The pellet is then subjected to a glycerol potassium-tartrate gradient for further purification. The virus-containing fraction is then diluted and pelleted by ultracentrifugation. The pellet is then dissolved in PBS and the virus is inactivated with C₃H₄O₂ (beta-propiolactone, BPL). Two rabbits are immunized subcutaneously (SC) on day 0, 14, and 2 8 with 1×10⁹ inactivated viral particles mixed with IFA as adjuvant. Rabbits are bled on days 0 (p,re-inoculation), 13, 28, and 35 (1 week after 3rd immunization). Sera obtained from this protocol were tested for their reactivity against SARS-CoV proteins in western blots and found to react with the major structural proteins spike (S), membrane (M), and nucleocapsid (N).

J. Emerging Coronavirus Vaccines

The SARS epidemic has lead to increased awareness of viral infections caused by coronaviruses. The vaccines of the invention may be adapted to prevent or treat emerging strains of coronavirus, including emerging strains of SARS virus.

The invention provides a vaccine comprising an inactivated (or killed) human coronavirus, an attenuated human coronavirus, a split human coronavirus preparation, or a recombinant or purified subunit formulation of one or more antigens from a human coronavirus, wherein the human coronavirus is not the SARS coronavirus. Optionally, the human coronavirus is not the 229E coronavirus. Optionally, the human coronavirus is not the OC43 coronavirus. Optionally, the human coronavirus is not the NL63 coronavirus. Thus the invention provides a vaccine as defined above, wherein the human coronavirus is not the SARS coronavirus, is not the 229E coronavirus, is not the OC43 coronavirus and is not the NL63 coronavirus. Such vaccines are useful for preventing and/or treating emerging human coronavirus infections.

The invention also provides a vaccine comprising: (a) an inactivated (or killed) human coronavirus, an attenuated human coronavirus, a split human coronavirus preparation, or a recombinant or purified subunit formulation of one or more antigens from a human coronavirus, wherein the human coronavirus is not the SARS coronavirus, as defined above; and (b) an inactivated (or killed) human coronavirus, an attenuated human coronavirus, a split human coronavirus preparation, or a recombinant or purified subunit formulation of one or more antigens from a human coronavirus, wherein the human coronavirus is the SARS coronavirus. Such vaccines are useful for preventing and/or treating both SARS and other human coronaviruses.

As well as providing vaccines comprising antigens from more than one type of coronavirus, the invention also provides vaccines comprising antigens from more than one strain of the same coronavirus e.g. different strains of the SARS coronavirus, or different strains of a coronavirus other than the SARS coronavirus. In one embodiment, the vaccine comprises antigens from at least two strains of coronavirus, or at least three strains of coronavirus. In one embodiment, the vaccine comprises antigens from at least two types of coronavirus. In one embodiment, the vaccine comprises at least one antigen from each of the known types of coronaviruses (type I, type II and type III). Such vaccines follow the model of current influenza vaccines.

The selection of coronaviruses and/or coronavirus strains for use in vaccines of the invention can be based on various criteria. For instance, selection may be based on viruses and/or strains that have been detected in the geographical region (e.g. northern or southern hemisphere, a particular country, etc.) where the vaccine targeted. Selection may be based on the results of animal surveillance e.g. of viruses detected in cat populations. Selection may be based on the results of clinical surveillance e.g. of viruses detected in patients hospitalized with respiratory infection. Selection may be performed every year e.g. prior to winter. Vaccines may also be administered yearly, again following the model of current influenza vaccines.

Preferred vaccines are sufficiently immunogenic to provide a neutralizing immune response, and more preferably a protective and/or therapeutic immune response. Particularly preferred vaccines meet the efficacy requirements that may be specified by the WHO from time to time.

A preferred subunit antigen for inclusion in vaccines of the invention is a purified spike protein, more preferably in oligomeric (e.g. trimeric) form. The spike protein may or my not be cleaved e.g. into its S1 and S2 products.

The techniques disclosed above for selecting viruses and/or strains for production of vaccines can also be used to select appropriate viruses and/or strains from which HR1 and HR2 sequences can be obtained for providing therapeutic peptides, as disclosed above.

III. Diagnostic Compositions and Methods of the Invention

The invention provides methods for detecting the SARS coronavirus. Detection in patient samples can be used to detect and diagnose infections by the virus. Detection in donated blood can be used to prevent inadvertent transmission of the virus during blood transplant procedures Detection methods fall into three main categories: detection of SARS virus nucleic acids; detection of SARS virus proteins; and detection of anti-SARS virus immune responses. The invention provides all such methods.

As used herein when referring to nucleotide sequences, particularly oligonucleotide probes and primers, “similar” sequences includes those sequences that are at least 90% identical to known SARSV genomic sequence and includes sequences that are at least 95% identical, at least 99% identical and 100% identical to the SARSV genomic sequence over the length of the probe or primer.

As used herein, the term “target nucleic acid region” or “target nucleic acid” denotes a nucleic acid molecule with a “target sequence” to be amplified. The target nucleic acid may be either single-stranded or double-stranded and may include other sequences besides the target sequence, which may not be amplified. The term “target sequence” refers to the particular nucleotide sequence of the target nucleic acid which is to be amplified. The target sequence may include a probe-hybridizing region contained within the target molecule with which a probe will form a stable hybrid under desired conditions. The “target sequence” may also include the complexing sequences to which the oligonucleotide primers complex and be extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands.

The term “primer” or “oligonucleotide primer” as used herein, refers to an oligonucleotide which acts to initiate synthesis of a complementary DNA strand when placed under conditions in which synthesis of a primer extension product is induced i.e. in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer is first treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “probe” or “oligonucleotide probe” refers to a structure comprised of a polynucleotide, as defined above, that contains a nucleic acid sequence complementary to a nucleic acid sequence present in the target nucleic acid analyte. The polynucleotide regions of probes may be composed of DNA, and/or RNA, and/or synthetic nucleotide analogs. When an “oligonucleotide probe” is to be used in a 5′ nuclease assay, such as the TaqMan™ technique, the probe will contain at least one fluorescer and at least one quencher which is digested by the 5′ endonuclease activity of a polymerase used in the reaction in order to detect any amplified target oligonucleotide sequences. In this context, the oligonucleotide probe will have a sufficient number of phosphodiester linkages adjacent to its 5′ end so that the 5′ to 3′ nuclease activity employed can efficiently degrade the bound probe to separate the fluorescers and quenchers. When an oligonucleotide probe is used in the TMA technique, it will be suitably labeled, as described below.

It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by e.g. the DNA polymerase to initiate DNA synthesis.

Stringent hybridization conditions will typically include salt concentrations of less than about 1 M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. Other factors may affect the stringency of hybridization, including base composition and length of the complementary strands, presence of organic solvents and extent of base mismatching, and the combination of parameters used is more important than the absolute measure of any one alone. Other hybridization conditions which may be controlled include buffer type and concentration, solution pH, presence and concentration of blocking reagents to decrease background binding such as repeat sequences or blocking protein solutions, detergent type(s) and concentrations, molecules such as polymers which increase the relative concentration of the polynucleotides, metal ion(s) and their concentration(s), chelator(s) and their concentrations, and other conditions known in the art. Less stringent, and/or more physiological, hybridization conditions are used where a labeled polynucleotide amplification product cycles on and off a substrate linked to a complementary probe polynucleotide during a real-time assay which is monitored during PCR amplification such as a molecular beacon assay. Such less stringent hybridization conditions can also comprise solution conditions effective for other aspects of the method, for example reverse transcription or PCR.

As used herein, a “biological sample” refers to a sample of tissue, cells or fluid isolated from a subject, that commonly includes antibodies produced by the subject. Typical samples include but are not limited to, blood, plasma, serum, fecal matter, urine, bone marrow, bile, spinal fluid, lymph fluid, samples of the skin, secretions of the skin, respiratory, intestinal, and genitourinary tracts, tears, saliva, sputum, mucous, milk, blood cells, organs, tissues, biopsies (e.g. lung, liver, kidney) and also samples of in vitro cell culture constituents including but not limited to conditioned media resulting from the growth of cells and tissues in culture medium e.g. recombinant cells, and cell components. Other samples that may be used for diagnosis include stool samples and nasopharyngeal aspirates.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fv molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); oligobodies; dimeric and trimeric antibody fragment constructs; minibodies (see, e.g., Packet al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and UK Patent Publication No. GB 2,276,169, published 21 Sep. 1994); and, any functional fragments obtained from such molecules, wherein such fragments retain specific-binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins.

Methods of making polyclonal and monoclonal antibodies are known in the art. Polyclonal antibodies are generated by immunizing a suitable animal, such as a mouse, rat, rabbit, sheep or goat, with an antigen of interest. In order to enhance immunogenicity, the antigen can be linked to a carrier prior to immunization. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are well known to those of ordinary skill in the art. Furthermore, the antigen may be conjugated to a bacterial toxoid, such as toxoid from diphtheria, tetanus, cholera, etc., in order to enhance the immunogenicity thereof.

Rabbits, sheep and goats are preferred for the preparation of polyclonal sera when large volumes of sera are desired. These animals are good design choices also because of the availability of labeled anti-rabbit, anti-sheep and anti-goat antibodies. Immunization is generally performed by mixing or emulsifying the antigen in saline, preferably in an adjuvant such as Freund's complete adjuvant (“FCA”), and injecting the mixture or emulsion parenterally (generally subcutaneously or intramuscularly). The animal is generally boosted 2-6 weeks later with one or more injections of the antigen in saline, preferably using Freund's incomplete adjuvant (“FIA”). Antibodies may also be generated by in vitro immunization, using methods known in the art. Polyclonal antisera is then obtained from the immunized animal.

Monoclonal antibodies are generally prepared using the method of Kohler & Milstein (1975) Nature 256:495-497, or a modification thereof, as described above.

Nucleic Acid Detection Methods

There are many well known methods of amplifying targeted sequences, such as the polymerase chain reaction (PCR), reverse transcription PCR (RT-PCR), the ligase chain reaction (LCR), the strand displacement amplification (SDA), and the nucleic acid sequence-based amplification (NASBA), transcription-mediated amplification (TMA) to name a few. These methods are described generally in the following references: (PCR) U.S. Pat. Nos. 4,683,195, 4,683,202, and 4,800,159; (RT-PCR) U.S. Pat. Nos. 5,310,652, 5,322,770; (LCR) EP Application No., 320,308 published Jun. 14, 1989; (SDA) U.S. Pat. Nos. 5,270,184, and 5,455,166 and “Empirical Aspects of Strand Displacement Amplification” by G. T. Walker in PCR Methods and Applications, 3(1):1-6 (1993), Cold Spring Harbor Laboratory Press; (TMA) U.S. Pat. No. 5,399,491, and (NASBA) “Nucleic Acid Sequence-Based Amplification (NASBA™)” by L. Malek et al., Ch. 36 in Methods in Molecular Biology, Vol. 28: Protocols for Nucleic Acid Analysis by Nonradioactive Probes, 1994 Ed. P. G. Isaac, Humana Press, Inc., Totowa, N.J. PCR methods may include variations that permit quantitation of the target sequence, for example, by real time PCR analysis (e.g., as described in U.S. Pat. Nos. 5,210,015, 5,487,972, 5,994,056, 6,171,785 inter alia). (Each of the above references are hereby incorporated by reference).

One embodiment of the method of the invention for detecting the presence of SARS virus in a sample comprises providing a sample suspected of containing a SARS virus nucleic acid target, amplifying a template sequence contained within said SARS virus nucleic acid target by any known technique of nucleic acid amplification, including any of those mentioned herein, using the oligonucleotide primers described herein, particularly those primers comprising the kits described herein, and detecting the amplified template sequence, wherein the presence of the amplified template sequence indicates the presence of SARS virus in said sample.

Amplification techniques generally involve the use of two primers. Where a target sequence is single-stranded, the techniques generally involve a preliminary step in which a complementary strand is made in order to give a double-stranded target. The two primers hybridize to different strands of the double-stranded target and are then extended. The extended products can serve as targets for further rounds of hybridization/extension. The net effect is to amplify a template sequence within the target, the 5′ and 3′ termini of the template being defined by the locations of the two primers in the target. As an alternative, if one or both of the primers contains a promoter sequence then the target can be amplified (by transcription) using a RNA polymerase (as in TMA).

The present invention provides methods and kits for amplifying and/or detecting a template or target sequence in the SARSV viral nucleic acid. The invention provides a kit comprising primers for amplifying a template sequence contained within a SARSV nucleic acid target, the kit comprising a first primer and a second primer, wherein the first primer comprises a sequence substantially complementary to a portion of said template sequence and the second primer comprises a sequence substantially complementary to a portion of the complement of said template sequence, wherein the sequences within said primers which have substantial complementarity define the termini of the template sequence to be amplified.

Kits of the invention may further comprise a probe which is substantially complementary to the template sequence and/or to its complement and which can hybridize thereto. This probe can be used in a hybridization technique to detect amplified template, or to isolate (i.e. “capture) the amplified template or the original target nucleic acid.

Kits of the invention may further comprise primers and/or probes for generating and detecting an internal standard, in order to aid quantitative measurements (e.g Fille et al. 1997 Biotechniques 23:34-36).

Kits of the invention may further comprise a DNA polymerase, which will generally be a thermostable DNA polymerase where a non-isothermal amplification process is to be used. The kits may also comprise supplies of dNTPs, a magnesium salt (e.g. MgCl₂), buffer solutions, etc.

Kits of the invention may comprise more than one pair of primers (e.g. for nested amplification), and one primer may be common to more than one primer pair. The kit may also comprise more than one probe.

Oligomer Probes and Primers

In connection with the nucleic acid detection methods of the present invention described above, oligomers having sequence similarity, or complementarity, to the SARSV genome are useful. The SARSV genome sequences mentioned herein may be used to produce probes and primers which can be used in assays for the detection of nucleic acids in test samples. The probes may be designed from conserved nucleotide regions of the polynucleotides of interest or from non-conserved nucleotide regions of the polynucleotide of interest. The design of such probes for optimization in assays is within the skill of those of ordinary skill in the art. Generally, nucleic acid probes are developed from non-conserved or unique regions when maximum specificity is desired, and nucleic acid probes are developed from conserved regions when assaying for nucleotide regions that are closely related to, for example, different members of a multi-gene family or in related species like mouse and man.

Using as a basis the SARSV genome which can be found as described herein, and/or preferably conserved regions of the SARSV genome, and/or the particularly described primer and probe sequences as disclosed herein, oligomers of approximately 8 nucleotides or more can be prepared which hybridize with the positive strand(s) of SARSV RNA or its complement, as well as to SARSV cDNAs. These oligomers can serve as probes for the detection (including isolation and/or labeling) of polynucleotides which contain SARSV nucleotide sequences, and/or as primers for the transcription and/or replication of targeted SARSV sequences. The oligomers contain a targeting polynucleotide sequence, which is comprised of nucleotides which are complementary to a target SARSV nucleotide sequence; the sequence is of sufficient length and complementarity with the SARSV sequence to form a duplex which has sufficient stability for the purpose intended. For example, if the purpose is the isolation, via immobilization, of an analyte containing a target SARSV sequence, the oligomers would contain a polynucleotide region which is of sufficient length and complementarity to the targeted SARSV sequence to afford sufficient duplex stability to immobilize the analyte on a solid surface, via its binding to the oligomers, under the isolation conditions. For example, also, if the oligomers are to serve as primers for the transcription and/or replication of target SARSV sequences in an analyte polynucleotide, the oligomers would contain a polynucleotide region of sufficient length and complementarity to the targeted SARSV sequence to allow the polymerizing agent to continue replication from the primers which are in stable duplex form with the target sequence, under the polymerizing conditions. For example, also, if the oligomers are to be used as label probes, or are to bind to multimers, the targeting polynucleotide region would be of sufficient length and complementarity to form stable hybrid duplex structures with the label probes and/or multimers to allow detection of the duplex. The oligomers may contain a minimum of about 4 contiguous nucleotides which are complementary to targeted SARSV sequence; usually the oligomers will contain a minimum of about 8 contiguous nucleotides which are complementary to the targeted SARSV sequence, and preferably will contain a minimum of about 14, 15, 16, 17, 18, 19, 20,21, 22, 23, 24, 25, 26, 27, 28, 29, or 30 contiguous nucleotides and up to about 50, 75, 100, 200 contiguous nucleotides or more, which are complementary to the targeted SARSV sequence.

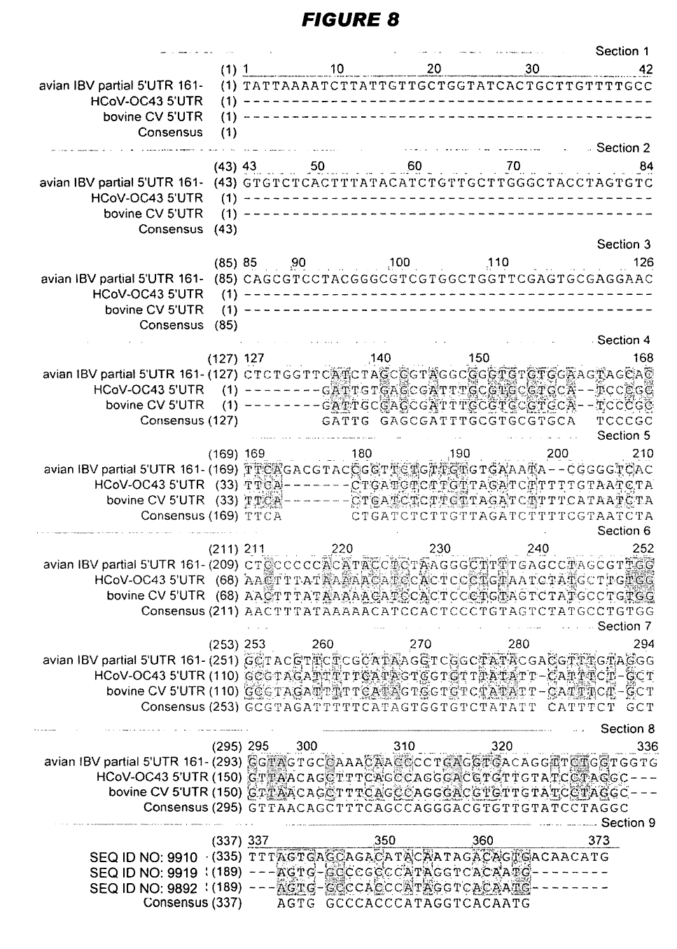

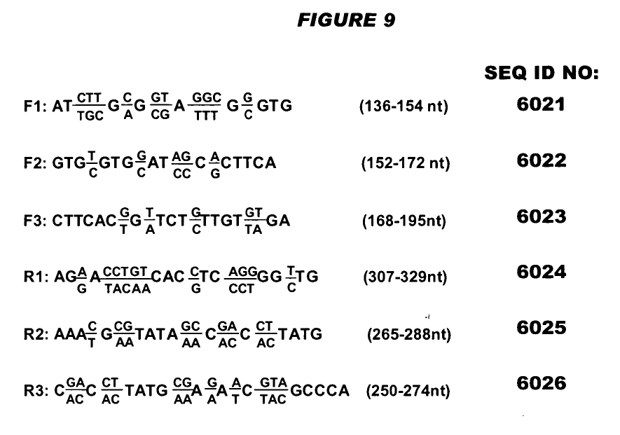

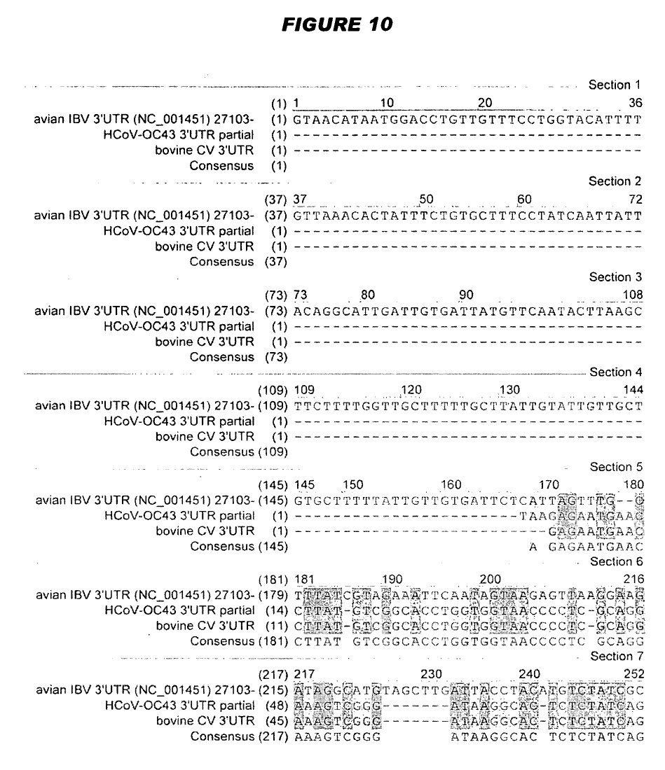

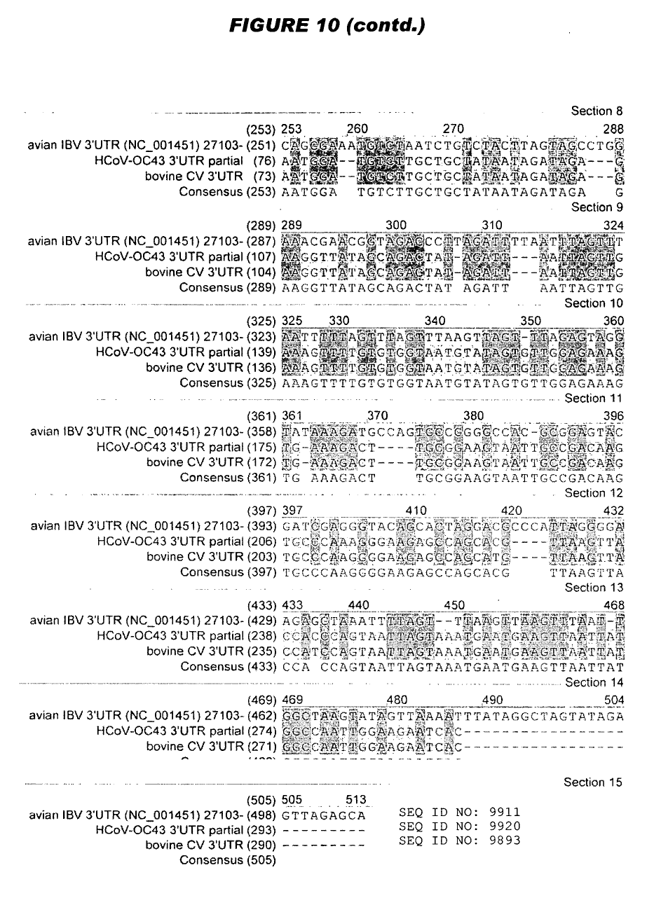

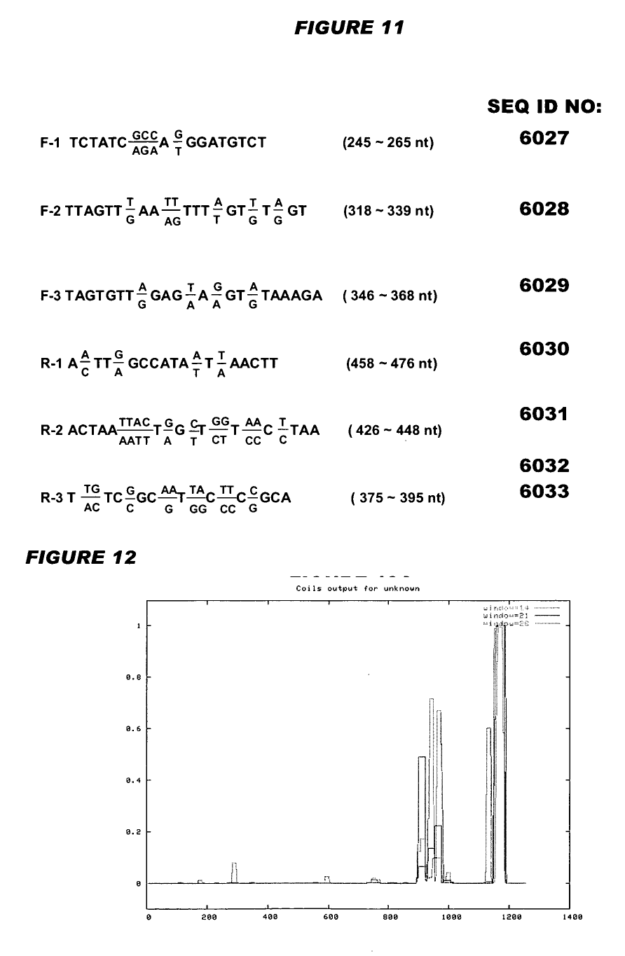

Typically, for use in the amplification based methods (for example, PCR, RT-PCR, TMA) oligomers will be used as primer sets such that one member of the primer set has sequence similarity or complementarity to a more conserved (among coronaviruses) portion of the SARSV genome and the other member of the primer set has sequence similarity or complementarity to a less conserved portion. The primer sets can be used to amplify the target region in ways that are well known in the art. Typically, the 5′ untranslated region (5′UTR) and the 3′ untranslated region (3′UTR) are among the most conserved regions. FIG. 8 shows an alignment of the 5′UTR of several coronaviruses. FIG. 10 shows an alignment of the 3′UTR of several coronaviruses. FIGS. 9 and 11 show the sequences of preferred primers for amplification of the 5′UTR and 3′UTR, respectively. Other primers and probes can readily be designed based on the sequence alignments provided herein.

The oligomer, however, need not consist only of the sequence which is complementary to the targeted SARSV sequence. It may contain in addition, nucleotide sequences (e.g. promoters) or other moieties which are suitable for the purposes for which the oligomers are used. For example, if the oligomers are used as primers for the amplification of SARSV sequences via, for example, PCR, they may contain sequences which, when in duplex, form restriction enzyme sites which facilitate the cloning of the amplified sequences. For example, also, if the oligomers are to be used as “capture probes” in hybridization assays, they would contain in addition a binding partner which is coupled to the oligomer containing the nucleotide sequence which is complementary to the targeted SARSV sequence. Other types of moieities or sequences which are useful of which the oligomers may be comprised or coupled to, are those which are known in the art to be suitable for a variety of purposes, including the labeling of nucleotide probes.

Table 4 (SEQ ID NOS: 1021-6020) shows forward and reverse primers that are useful for nucleic acid amplification of SARSV for diagnostic and screening methods.

Preferred primers and probes for SARS nucleic acid detection for diagnostic and screening are SEQ ID NOS: 7332-7336 (forward primers), SEQ ID NOS: 7337-7341 (reverse primers) and SEQ ID NOS: 7342-7352 (probes). These primers and probes are useful for detection of sequences in the 3′ UTR.

Any of the above forward primers may be used in combination with any of the above reverse primers for amplification of SARSV nucleic acid. The amplified product may be detected (or captured) with any of the above probes. Particularly preferred combinations of forward and reverse primers and the probes for detecting the amplified product include: Forward SEQ ID NO: 7332 with reverse SEQ ID NO: 7337, 7338, 7339 or 7341 and probe SEQ ID NO: 7342; forward SEQ ID NO: 7333 or 7334 with reverse SEQ ID NO: 7340 and any of probes SEQ ID NO: 7343-7351; Forward SEQ ID NO: 7335 and reverse SEQ ID NO: 7340 or 7341 and any of probes SEQ ID NO: 7342-7352. Other combinations of forward and reverse primers and appropriate probes can readily be determined by those skilled in the art from the above information.

Additional preferred primers and probes for SARS nucleic acid detection for diagnostic and screening are SEQ ID NOS: 7353-7362 (forward primers), SEQ ID NOS: 7363-7373 (reverse primers) and SEQ ID NOS: 7374-7385 (probes). The primers and probes are useful for detection of sequences in the 5′ UTR.

The above primers may be used in combination for amplification of SARSV nucleic acid as follows: any of forward primers SEQ ID NO: 7353-7356 with any of reverse primers SEQ ID NO: 7363-7366, 7368 and the amplified product detected (or captured) with probes SEQ ID NO: 7374; any of forward primers SEQ ID NO: 7357-7362 with any of reverse primers SEQ ID NO: 7367, 7369-7373 and the amplified products detected (or captured) with any of probes SEQ ID NO: 7375-7385. Particularly preferred combinations of forward and reverse primers and probes are: Forward primers SEQ ID NO: 7353-7356 with any of reverse primers SEQ ID NO: 7363-7366 and probes SEQ ID NO: 7374; forward primers SEQ ID NO: 7357-7358 with reverse primers SEQ ID NO: 7367, 7369 and probes SEQ ID NO: 7375 or 7376; Forward primers SEQ ID NO: 7357-7359 with reverse primers SEQ ID NO: 7367, 7369 or 7370 and probe SEQ ID NO: 7375 or 7376. More preferred are combinations of SEQ ID NO: 7353 or 7354 with SEQ ID NO: 7363 or 7364 and probe SEQ ID NO: 7374. Other combinations of forward and reverse primers and appropriate probes can readily be determined by those skilled in the art from the above information. A particularly conserved octanucleotide sequence (SEQ ID NO: 7386) occurs in the 3′UTR of SARS (approximately 70-80 bases from the 3′ end) and of several other Coronaviruses that may be particularly useful in identifying SARSV. Primers including in this region are preferably combined with reverse primers from regions of sequence that are more specific for SARS.

In addition to the above, the intergenic sequence (IS) that is characteristic of Coronavirus has been identified in SARSV (see above). The IS minimally comprises the sequence ACGAAC (SEQ ID NO: 7293) which occurs upstream of each open reading frame (ORF) in the viral genome. The 5′UTR which includes the IS is spliced onto the 5′ end of each viral mRNA at or adjacent to the site of the IS. Thus, primers comprising the IS or its complement are useful for amplifying viral nucleic acids, including cDNA made from the viral RNAs. The invention thus comprises a set of primers in which one primer comprises ACGAAC (SEQ ID NO: 7293) or its complement (SEQ ID NO: 7387) and one primer comprises any appropriate sequence from the SARS genome, or a complementary sequence. Useful probes for detecting and/or capturing the viral RNAs or cDNA made from the viral RNAs may also comprise the IS sequence, or its complement, described above.

One set of primers for amplification of SARS sequences, particularly by RT-PCR, uses SEQ ID NOs 6562, 6563, 6564 and 6565. Of these, 6562 & 6564 are sense primers and 6563 & 6565 are antisense primers. Primers SEQ ID NOS: 6562 & 6565 may be used in a first amplification, with a second nested amplification being performed using primers SEQ ID NOS: 6563 & 6564. In some embodiments of the invention, these four primers are excluded.

One kit for amplification and detection of SARS sequences, particularly by RT-PCR, uses SEQ ID NOs 6567 & 6568 as primers, and SEQ ID NO 6566 as a probe (typically labeled e.g. with TAMRA and/or FAM) for the amplified sequence. In some embodiments of the invention, these primers and probe are excluded.

One kit for amplification and detection of SARS sequences, particularly by RT-PCR, uses SEQ if NOs 7395 & 6568 as primers, and SEQ ID NO 6566 as a probe (typically labeled e.g. with TAMRA and/or FAM) for the amplified sequence. In some embodiments of the invention, these primers and probe are excluded.

One kit for amplification of SARS sequences, particularly the nucleocapsid gene, uses SEQ ID NOs 6560 & 6561 as primers. In some embodiments of the invention, these primers are excluded.

One kit for amplification of SARS sequences uses SEQ ID NOs 6496, 6497, 6562, 6563, 6564 & 6565 as primers. In some embodiments of the invention, these primers are excluded.

One kit for amplification of SARS sequences uses SEQ ID NOs 6562, 6563, 6564 & 6565 as primers. In some embodiments of the invention, these primers are excluded.

One kit for amplification of SARS sequences uses SEQ ID NOs 6500, 6501, 6502 & 6503 as primers. In some embodiments of the invention, these primers are excluded.

One kit for amplification of SARS sequences uses SEQ ID NOs 6496, 6497, 6500, 6501, 6502, 6503, 6562, 6563, 6564 & 6565 as primers. In some embodiments of the invention, these primers are excluded.

One kit for amplification and detection of SARS sequences, particularly by realtime (e.g. TaqMan™) PCR, uses SEQ ID NOs 6567 & 6568 as primers, and SEQ ID NO 6566 as a probe (typically labeled e.g. with TAMRA and/or FAM) for the amplified sequence. In some embodiments of the invention, these primers and probe are excluded.

One kit for amplification and detection of SARS sequences, particularly by realtime (e.g. TaqMan™) PCR, uses SEQ ID NOs 7395 & 6568 as primers, and SEQ ID NO 6566 as a probe (typically labeled e.g. with TAMRA and/or FAM) for the amplified sequence. In some embodiments of the invention, these primers and probe are excluded.

One kit for amplification and detection of SARS sequences uses SEQ ID NOs 6562, 6565 and 6568 as primers, and SEQ ID NOs 7396 and 7397 as probes (typically labeled e.g. with TAMRA and/or FAM) for the amplified sequence. In some embodiments of the invention, these primers and probe are excluded.

One kit for amplification and detection of SARS sequences uses an oligonucleotide comprising SEQ ID NO: 9780 as a forward primer, an oligonucleotide comprising SEQ ID NO: 9781 as a reverse primer, and an oligonucleotide comprising SEQ ID NO: 9782 as a probe.

Preferred sequences for use with RT-PCR and LightCycler analysis include SEQ ID NOs 6562, 6568, 6565, 7396 & 7397. In some embodiments of the invention, these primers and probe are excluded.

The preparation of the oligomers is by means known in the art, including, for example, by methods which include excision, transcription, or chemical synthesis. The target sequences and/or regions of the genome which are selected to which the targeting polynucleotides of the oligomers are complementary depend upon the purpose. For example, if the goal is to screen for the presence of SARSV in biological samples (e.g. blood, respiratory material, liver, lung), the preferred oligomers would be used as probes and/or primers, and would hybridize to conserved regions of the SARSV genome. Some of the conserved regions of the SARSV genome to which the oligomers may bind are described herein, for example, 5′UTR and 3′UTR.

In the basic nucleic acid hybridization assay, single-stranded analyte nucleic acid (either DNA or RNA) is hybridized to a nucleic acid probe, and resulting duplexes are detected. The probes for SARSV polynucleotides (natural or derived) are a length which allows the detection of unique viral sequences by hybridization. While 6-8 nucleotides may be a workable length, sequences of 10-12 nucleotides are preferred, and about 13, 14, 15, 16, 17, 18, 19, 20, or 21 or more nucleotides or more appears optimal. Preferably, these sequences will derive from regions which lack heterogeneity. These probes can be prepared using routine methods, including automated oligonucleotide synthetic methods. Among useful probes, for example, are those derived from less conserved regions of the SARSV genome. Regions of the genome that are typically less conserved can be readily ascertained from the sequence alignments provided herein, as well as by any other well known techniques. A complement to any unique portion of the SARSV genome will be satisfactory. For use as probes, complete complementarity is desirable, though it may be unnecessary as the length of the fragment is increased.

For use of such probes as agents to detect the presence of SARSV polynucleotides (for example in screening for contaminated blood or for diagnosing infected individuals), the biological sample to be analyzed, such as, without limitation, blood, serum, lung, liver, mucous, kidney, saliva, or sputum, may be treated, if desired, to extract the nucleic acids contained therein. The resulting nucleic acid from the sample may be subjected to gel electrophoresis or other size separation techniques; alternatively, the nucleic acid sample may be dot blotted without size separation. In order to form hybrid duplexes with the targeting sequence of the probe, the targeted region of the analyte nucleic acid must be in single stranded form. Where the sequence is naturally present in single stranded form, denaturation will not be required. However, where the sequence is present in double stranded form, the sequence will be denatured. Denaturation can be carried out by various techniques known in the art. Subsequent to denaturation, the analyte nucleic acid and probe are incubated under conditions which promote stable hybrid formation of the target sequence in the probe with the putative targeted sequence in the analyte, and the resulting duplexes containing the probe(s) are detected.

Detection of the resulting duplex, if any, is usually accomplished by the use of labeled probes; alternatively, the probe may be unlabeled, but may be detectable by specific binding with a ligand which is labeled, either directly or indirectly. Suitable labels, and methods for labeling probes and ligands are known in the art, and include, for example, radioactive labels which may be incorporated by known methods (e.g., nick translation or kinasing), biotin, fluorescent groups, chemiluminescent groups (e.g., dioxetanes, particularly triggered dioxetanes), enzymes, antibodies, and the like.

The region of the probes which are used to bind to the analyte can be made completely complementary to the SARSV genome. Therefore, usually high stringency conditions are desirable in order to prevent false positives. However, conditions of high stringency should only be used if the probes are complementary to regions of the viral genome which lack heterogeneity. The stringency of hybridization is determined by a number of factors during hybridization and during the washing procedure, including temperature, ionic strength, length of time, and concentration of formamide. These factors are outlined in, for example, Maniatis T. (1982).

Variations of this basic scheme which are known in the art, including those which facilitate separation of the duplexes to be detected from extraneous materials and/or which amplify the signal from the labeled moiety, may also be used. A number of these variations are reviewed in, for example: Matthews & Kricka (1988), Analytical Biochemistry 169:1; Landegren et al. (1988), Science 242:229; and Mittlin (1989), Clinical Chem. 35:1819. These and the following publications describing assay formats are hereby incorporated by reference herein. Probes suitable for detecting SARSV in these assays are comprised of sequences which hybridize with target SARSV polynucleotide sequences to form duplexes with the analyte strand, wherein the duplexes are of sufficient stability for detection in the specified assay system.

A suitable variation is, for example, one which is described in U.S. Pat. No. 4,868,105, issued Sep. 9, 1989, and in EPO Publication No. 225,807 (published Jun. 16, 1987). These publications describe a solution phase nucleic acid hybridization assay in which the analyte nucleic acid is hybridized to a labeling probe set and to a capturing probe set. The probe-analyte complex is coupled by hybridization with a solid-supported capture probe that is complementary to the capture probe set. This permits the analyte nucleic acid to be removed from solution as a solid phase complex. Having the analyte in the form of a solid phase complex facilitates subsequent separation steps in the assay. The labeling probe set is complementary to a labeled probe that is bound through hybridization to the solid phase/analyte complex.

The polymerase chain reaction (PCR) is a technique for amplifying a desired nucleic acid sequence (target) contained in a nucleic acid or mixture thereof. In PCR, a pair of primers are employed in excess to hybridize to the complementary strands of the target nucleic acid. The primers are each extended by a polymerase using the target nucleic acid as a template. The extension products become target sequences themselves, following dissociation from the original target strand. New primers then are hybridized and extended by a polymerase, and the cycle is repeated to geometrically increase the number of target sequence molecules. PCR is disclosed in U.S. Pat. Nos. 4,683,195 and 4,683,202, which are incorporated herein by reference.

The Ligase Chain Reaction (LCR) is an alternate method for nucleic acid amplification. In LCR, probe pairs are used which include two primary (first and second) and two secondary (third and fourth) probes, all of which are employed in molar excess to target. The first probe hybridizes to a first segment of the target strand, and the second probe hybridizes to a second segment of the target strand, the first and second segments being contiguous so that the primary probes abut one another in 5′ phosphate-3′ hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. Of course, if the target is initially double stranded, the secondary probes also will hybridize to the target complement in the first instance. Once the ligated strand of primary probes is separated from the target strand, it will hybridize with the third and fourth probes which can be ligated to form a complementary, secondary ligated product. It is important to realize that the ligated products are functionally equivalent to either the target or its complement. By repeated cycles of hybridization and ligation, amplification of the target sequence is achieved. This technique is described more completely in EP-A-320 308 to K. Backman published Jun. 16, 1989 and EP-A-0439182 to K. Backman et al., published Jul. 31, 1991, both of which are incorporated herein by reference.

For amplification of mRNAs, it is within the scope of the present invention to reverse transcribe mRNA into cDNA followed by polymerase chain reaction (RT-PCR); or, to use a single enzyme for both steps as described in U.S. Pat. No. 5,322,770, which is incorporated herein by reference; or reverse transcribe mRNA into cDNA followed by asymmetric gap ligase chain reaction (RT-AGLCR) as described by R. L. Marshall et al., PCR Methods and Applications 4:80-84 (1994), which also is incorporated herein by reference.

TMA is described in detail in, e.g., U.S. Pat. No. 5,399,491, the disclosure of which is incorporated herein by reference in its entirety. In one example of a typical assay, an isolated nucleic acid sample, suspected of containing a SARSV target sequence, is mixed with a buffer concentrate containing the buffer, salts, magnesium, nucleotide triphosphates, primers, dithiothreitol, and spermidine. The reaction is optionally incubated at about 100° C. for approximately two minutes to denature any secondary structure. After cooling to room temperature, reverse transcriptase, RNA polymerase, and RNAse H are added and the mixture is incubated for two to four hours at 37° C. The reaction can then be assayed by denaturing the product, adding a probe solution, incubating 20 minutes at 60° C., adding a solution to selectively hydrolyze the unhybridized probe, incubating the reaction six minutes at 60° C., and measuring the remaining chemiluminescence in a luminometer.

Generally, TMA includes the following steps: (a) isolating nucleic acid, including RNA, from the biological sample of interest suspected of being infected with SARSV; and (b) combining into a reaction mixture (i) the isolated nucleic acid, (ii) first and second oligonucleotide primers, the first primer having a complexing sequence sufficiently complementary to the 3′ terminal portion of an RNA target sequence, if present (for example the (+) strand), to complex therewith, and the second primer having a complexing sequence sufficiently complementary to the 3′ terminal portion of the target sequence of its complement (for example, the (−) strand) to complex therewith, wherein the first oligonucleotide further comprises a sequence 5′ to the complexing sequence which includes a promoter, (iii) a reverse transcriptase or RNA and DNA dependent DNA polymerases, (iv) an enzyme activity which selectively degrades the RNA strand of an RNA-DNA complex (such as an RNAse H) and (v) an RNA polymerase which recognizes the promoter.

The components of the reaction mixture may be combined stepwise or at once. The reaction mixture is incubated under conditions whereby an oligonucleotide/target sequence is formed, including DNA priming and nucleic acid synthesizing conditions (including ribonucleotide triphosphates and deoxyribonucleotide triphosphates) for a period of time sufficient to provide multiple copies of the target sequence. The reaction advantageously takes place under conditions suitable for maintaining the stability of reaction components such as the component enzymes and without requiring modification or manipulation of reaction conditions during the course of the amplification reaction. Accordingly, the reaction may take place under conditions that are substantially isothermal and include substantially constant ionic strength and pH. The reaction conveniently does not require a denaturation step to separate the RNA-DNA complex produced by the first DNA extension reaction.

Suitable DNA polymerases include reverse transcriptases, such as avian myeloblastosis virus (AMV) reverse transcriptase (available from, e.g., Seikagaku America, Inc.) and Moloney murine leukemia virus (MMLV) reverse transcriptase (available from, e.g., Bethesda Research Laboratories).

Promoters or promoter sequences suitable for incorporation in the primers are nucleic acid sequences (either naturally occurring, produced synthetically or a product of a restriction digest) that are specifically recognized by an RNA polymerase that recognizes and binds to that sequence and initiates the process of transcription whereby RNA transcripts are produced. The sequence may optionally include nucleotide bases extending beyond the actual recognition site for the RNA polymerase which may impart added stability or susceptibility to degradation processes or increased transcription efficiency. Examples of useful promoters include those which are recognized by certain bacteriophage polymerases such as those from bacteriophage T3, T7 or SP6, or a promoter from E. coli. These RNA polymerases are readily available from commercial sources, such as New England Biolabs and Epicentre.

Some of the reverse transcriptases suitable for use in the methods herein have an RNAse H activity, such as AMV reverse transcriptase. It may, however, be preferable to add exogenous RNAse H, such as E. coli RNAse H, even when AMV reverse transcriptase is used. RNAse H is readily available from, e.g., Bethesda Research Laboratories.

The RNA transcripts produced by these methods may serve as templates to produce additional copies of the target sequence through the above-described mechanisms. The system is autocatalytic and amplification occurs autocatalytically without the need for repeatedly modifying or changing reaction conditions such as temperature, pH, ionic strength or the like.

Detection may be done using a wide variety of methods, including direct sequencing, hybridization with sequence-specific oligomers, gel electrophoresis and mass spectrometry. these methods can use heterogeneous or homogeneous formats, isotopic or nonisotopic labels, as well as no labels at all.

Suitable labeling moieties for attachment to primers and/or to probes used in methods of the invention include, but are not limited to: 5-FAM (also called 5-carboxyfluorescein; also called Spiro(isobenzofuran-1(3H), 9′-(9H)xanthene)-5-carboxylic acid,3′,6′-dihydroxy-3-oxo-6-carboxyfluorescein); 5-Hexachloro-Fluorescein ([4,7,2′,4′,5′,7′-hexachloro-(3′,6′-dipivaloylfluoresceinyl)-6-carboxylic acid]); 6-Hexachloro-Fluorescein ([4,7,2′,4′,5′,7′-hexachloro-(3′,6′-dipivaloylfluoresceinyl)-5-carboxylic acid]); 5-Tetrachloro-Fluorescein ([4,7,2′,7′-tetrachloro-(3′,6′-dipivaloylfluoresceinyl)-5-carboxylic acid]); 6-Tetrachloro-Fluorescein ([4,7,2′,7′-tetrachloro-(3′,6′-dipivaloylfluoresceinyl)-6-carboxylic acid]); tetramethylrhodamines (TAMRA), including (i) 5-TAMRA (5-carboxytetramethylrhodamine; Xanthylium, 9-(2,4-dicarboxyphenyl)-3,6-bis(dimethylamino) and (ii) 6-TAMRA (6-carboxytetramethylrhodamine; Xanthylium, 9-(2,5-dicarboxyphenyl)-3,6-bis(dimethylamino); EDANS (5-((2-aminoethyl)amino)naphthalene-1-sulfonic acid); 1,5-IAEDANS (5-((((2-iodoacetyl)amino)ethyl)amino)naphthalene-1-sulfonic acid); DABCYL (4-((4-(dimethylamino)phenyl)azo)benzoic acid); Cy5 (Indodicarbocyanine-5); Cy3 (Indodicarbocyanine-3); and BODIPY™ FL (4,4-difluoro-5,7-dimethyl-4-bora-3a,4a-diaza-s-indacene-3-propionic acid). Labelling of probes with both FAM (e.g. at 5′) and TAMRA (e.g. at 3′) is preferred.

Nucleic acids of the invention may be used in solution or may be bound to a solid matrix or support e.g. in the format of a DNA array,

As is readily apparent, design of the assays described herein are subject to a great deal of variation, and many formats are known in the art. The above descriptions are merely provided as guidance and one of skill in the art can readily modify the described protocols, using techniques well known in the art.

One 302nt amplicon of the SARS virus is known as “BNI-1” (SEQ ID NO: 9927). It was sequenced at the Bernhard Nocht Institute, Hamburg, Germany. In April 2003 the BNI-1 sequence was published on the WHO website (http://www.who.int/csr/sars/primers/en/) and in Dorsten et al., “Identification of a Novel Coronavirus in Patients with Severe Acute Respiratory Syndrome”, New England Journal of Medicine, published online at http://www.nejm.org. Both references are incorporated herein by reference in their entirety. Some embodiments of the invention do not encompass a nucleic acid consisting of SEQ ID NO: 9927. Some other embodiments of the invention do not encompass a nucleic acid comprising SEQ ID NO: 9927. Some embodiments of the invention do not encompass a polypeptide consisting of any one of SEQ ID NO^{S}: 9928 to 9959. Some other embodiments of the invention do not encompass a nucleic acid comprising any one of SEQ ID NOs: 9928 to 9959. Some embodiments of the invention are not subject to these exclusions.

Immunoassays

The present invention utilizes various immunoassay techniques for identifying individuals exposed to SARSV and/or biological samples containing SARSV antigens or antibodies to SARSV.

Immunoassay Formats

The SARSV antigens may be employed in virtually any assay format that employs a known antigen to detect antibodies. A common feature of all of these assays is that the antigen is contacted with biological sample suspected of containing SARSV antibodies under conditions that permit the antigen to bind to any such antibody present in the component. Such conditions will typically be physiologic temperature, pH and ionic strength using an excess of antigen. The incubation of the antigen with the specimen is followed by detection of immune complexes comprised of the antigen. Alternatively, anti-SARSV antibodies may be employed to detect the presence of SARSV antigens in a biological sample. Combination antigen/antibody assays are also contemplated; for example, as described for HCV detection in US patent 6,630,298.

Design of the immunoassays is subject to a great deal of variation, and many formats are known in the art. Protocols may, for example, use solid supports, or immunoprecipitation. Most assays involve the use of labeled antibody or polypeptide; the labels may be, for example, enzymatic, fluorescent, chemiluminescent, radioactive, or dye molecules. Assays which amplify the signals from the immune complex are also known; examples of which are assays which utilize biotin and avidin, and enzyme-labeled and mediated immunoassays, such as ELISA assays.

The immunoassay may be, without limitation, in a heterogeneous or in a homogeneous format, and of a standard or competitive type. In a heterogeneous format, the polypeptide is typically bound to a solid matrix or support to facilitate separation of the sample from the polypeptide after incubation. Examples of solid supports that can be used are nitrocellulose (e.g., in membrane or microtiter well form), polyvinyl chloride (e.g., in sheets or microtiter wells), polystyrene latex (e.g., in beads or microtiter plates, polyvinylidine fluoride, diazotized paper, nylon membranes, microchips, high or low density biochips, recombinant immunoassays (RIBA), microfluidity devices, micromagnetic beads, activated beads, and Protein A beads. For example, Dynatech Immunlon or Immunlon 2 microtiter plates or 0.25 inch polystyrene beads (Precision Plastic Ball) can be used in the heterogeneous format. The solid support containing the antigenic polypeptides is typically washed after separating it from the test sample, and prior to detection of bound antibodies. Both standard and competitive formats are known in the art.

In a homogenous format, the test sample is incubated with the combination of antigens in solution. For example, it may be under conditions that will precipitate any antigen-antibody complexes which are formed. Both standard and competitive formats for these assays are known in the art.

In a standard format, the amount of SARSV antibodies in the antibody-antigen complexes is directly monitored. This may be accomplished by determining whether labeled anti-xenogeneic (e.g., anti-human) antibodies which recognize an epitope on anti-SARSV antibodies will bind due to complex formation. In a competitive format, the amount of SARSV antibodies in the sample is deduced by monitoring the competitive effect on the binding of a known amount of labeled antibody (or other competing ligand) in the complex.

Complexes formed comprising anti-SARSV antibody (or in the case of competitive assays, the amount of competing antibody) are detected by any of a number of known techniques, depending on the format. For example, unlabeled SARSV antibodies in the complex may be detected using a conjugate of antixenogeneic Ig complexed with a label, (e.g., an enzyme label).

In an immunoprecipitation or agglutination assay format the reaction between the SARSV antigens and the antibody forms a network that precipitates from the solution or suspension and forms a visible layer or film of precipitate. If no anti-SARSV antibody is present in the test specimen, no visible precipitate is formed.

There are at least three specific types of particle agglutination (PA) assays. These assays are used for the detection of antibodies to various antigens when coated to a support. One type of this assay is the hemagglutination assay using red blood cells (RBCs) that are sensitized by passively adsorbing antigen (or antibody) to the RBC. The addition of specific antigen antibodies present in the body component, if any, causes the RBCs coated with the purified antigen to agglutinate.

To eliminate potential non-specific reactions in the hemagglutination assay, two artificial carriers may be used instead of RBC in the PA. The most common of these are latex particles. However, gelatin particles may also be used. The assays utilizing either of these carriers are based on passive agglutination of the particles coated with purified antigens.

The SARSV antigens will typically be packaged in the form of a kit for use in these immunoassays. The kit will normally contain in separate containers the native SARSV antigen, control antibody formulations (positive and/or negative), labeled antibody when the assay format requires same and signal generating reagents (e.g., enzyme substrate) if the label does not generate a signal directly. The native SARSV antigen may be already bound to a solid matrix or separate with reagents for binding it to the matrix. Instructions (e.g., written, tape, CD-ROM, etc.) for carrying out the assay usually will be included in the kit.

Immunoassays that utilize the native SARSV antigen are additionally useful in screening blood for the preparation of a supply from which potentially infective SARSV is lacking. The method for the preparation of the blood supply comprises the following steps. Reacting a body component, preferably blood or a blood component, from the individual donating blood with native SARSV antigen to allow an immunological reaction between SARSV antibodies, if any, and the SARSV antigen. Detecting whether anti-SARSV antibody-SARSV antigen complexes are formed as a result of the reacting. Blood contributed to the blood supply is from donors that do not exhibit antibodies to the native SARSV antigens.

Production of Antibodies

As explained above, the assay may utilize various antibodies which may be bound to a solid support, and that detect antigen or antigen/antibody complexes formed when SARSV infection is present in the sample. These antibodies may be polyclonal or monoclonal antibody preparations, monospecific antisera, human antibodies, or may be hybrid or chimeric antibodies, such as humanized antibodies, altered antibodies, F(ab′)₂ fragments, F(ab) fragments, Fv fragments, single-domain antibodies, dimeric or trimeric antibody fragment constructs, minibodies, or functional fragments thereof which bind to the antigen in question.

Antibodies are produced using techniques well known to those of skill in the art and disclosed in, for example, U.S. Pat. Nos. 4,011,308; 4,722,890; 4,016,043; 3,876,504; 3,770,380; and 4,372,745. For example, polyclonal antibodies are generated by immunizing a suitable animal, such as a mouse, rat, rabbit, sheep or goat, with an antigen of interest. In order to enhance immunogenicity, the antigen can be linked to a carrier prior to immunization. Such carriers are well known to those of ordinary skill in the art. Immunization is generally performed by mixing or emulsifying the antigen in saline, preferably in an adjuvant such as Freund's complete adjuvant, and injecting the mixture or emulsion parenterally (generally subcutaneously or intramuscularly). The animal is generally boosted 2-6 weeks later with one or more injections of the antigen in saline, preferably using Freund's incomplete adjuvant. Antibodies may also be generated by in vitro immunization, using methods known in the art. Polyclonal antiserum is then obtained from the immunized animal.

Monoclonal antibodies are generally prepared using the method of Kohler & Milstein (1975) Nature 256:495-497, or a modification thereof, as described above.

As explained above, antibody fragments which retain the ability to recognize the antigen of interest, will also find use in the subject immunoassays. A number of antibody fragments are known in the art which comprise antigen-binding sites capable of exhibiting immunological binding properties of an intact antibody molecule. For example, functional antibody fragments can be produced by cleaving a constant region, not responsible for antigen binding, from the antibody molecule, using e.g., pepsin, to produce F(ab′)₂ fragments. These fragments will contain two antigen binding sites, but lack a portion of the constant region from each of the heavy chains. Similarly, if desired, Fab fragments, comprising a single antigen binding site, can be produced, e.g., by digestion of polyclonal or monoclonal antibodies with papain. Functional fragments, including only the variable regions of the heavy and light chains, can also be produced, using standard techniques such as recombinant production or preferential proteolytic cleavage of immunoglobulin molecules. These fragments are known as Fv. See, e.g., Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single-chain Fv (“sFv” or “scFv”) polypeptide is a covalently linked V_{H}-V_{L} heterodimer which is expressed from a gene fusion including V_{H}- and V_{L}-encoding genes linked by a peptide-encoding linker. Huston et al. (1988) Proc. Nat. Acad. Sci. USA 85:5879-5883. A number of methods have been described to discern and develop chemical structures (linkers) for converting the naturally aggregated, but chemically separated, light and heavy polypeptide chains from an antibody V region into an sFv molecule which will fold into a three dimensional structure substantially similar to the structure of an antigen-binding site. See, e.g., U.S. Pat. Nos. 5,091,513, 5,132,405 and 4,946,778. The sFv molecules may be produced using methods described in the art. See, e.g., Huston et al. (1988) Proc. Nat. Acad. Sci. USA 85:5879-5883; U.S. Pat. Nos. 5,091,513, 5,132,405 and 4,946,778. Design criteria include determining the appropriate length to span the distance between the C-terminus of one chain and the N-terminus of the other, wherein the linker is generally formed from small hydrophilic amino acid residues that do not tend to coil or form secondary structures. Such methods have been described in the art. See, e.g., U.S. Pat. Nos. 5,091,513, 5,132,405 and 4,946,778. Suitable linkers generally comprise polypeptide chains of alternating sets of glycine and serine residues, and may include glutamic acid and lysine residues inserted to enhance solubility.

“Mini-antibodies” or “minibodies” will also find use with the present invention. Minibodies are sFv polypeptide chains which include oligomerization domains at their C-termini, separated from the sFv by a hinge region. Pack et al. (1992) Biochem 31:1579-1584. The oligomerization domain comprises self-associating a-helices, e.g., leucine zippers, that can be further stabilized by additional disulfide bonds. The oligomerization domain is designed to be compatible with vectorial folding across a membrane, a process thought to facilitate in vivo folding of the polypeptide into a functional binding protein. Generally, minibodies are produced using recombinant methods well known in the art. See, e.g., Pack et al. (1992) Biochem 31:1579-1584; Cumber et al. (1992) J. Immunology 149B: 120-126.

Production of SARS Antigens

The SARSV antigens used in the present invention are generally produced recombinantly. Thus, polynucleotides encoding SARSV antigens for use with the present invention can be made using standard techniques of molecular biology. For example, polynucleotide sequences coding for the above-described molecules can be obtained using recombinant methods, such as by screening cDNA and genomic libraries from cells expressing the gene, or by deriving the gene from a vector known to include the same. Furthermore, the desired gene can be isolated directly from viral nucleic acid molecules, using techniques described in the art, such as those described for HCV in Houghton et al., U.S. Pat. No. 5,350,671. The gene encoding the antigen of interest can also be produced synthetically, rather than cloned. The molecules can be designed with appropriate codons for the particular sequence (preferably optimum codons for the expression host of choice). The complete sequence is then assembled from overlapping oligonucleotides prepared by standard methods and assembled into a complete coding sequence. See, e.g., Edge (1981) Nature 292:756; Nambair et al. (1984) Science 223:1299; and Jay et al. (1984) J. Biol. Chem. 259:6311.

Thus, particular nucleotide sequences can be obtained from vectors harboring the desired sequences or synthesized completely or in part using various oligonucleotide synthesis techniques known in the art, such as site-directed mutagenesis and polymerase chain reaction (PCR) techniques where appropriate. See, e.g., Sambrook, supra. In particular, one method of obtaining nucleotide sequences encoding the desired sequences is by annealing complementary sets of overlapping synthetic oligonucleotides produced in a conventional, automated polynucleotide synthesizer, followed by ligation with an appropriate DNA ligase and amplification of the ligated nucleotide sequence via PCR. See, e.g., Jayaraman et al. (1991) Proc. Natl. Acad. Sci. USA 88:4084-4088. Additionally, oligonucleotide directed synthesis (Jones et al. (1986) Nature 54:75-82), oligonucleotide directed mutagenesis of pre-existing nucleotide regions (Riechmann et al. (1988) Nature 332:323-327 and Verhoeyen et al. (1988) Science 239:1534-1536), and enzymatic filling-in of gapped oligonucleotides using T4 DNA polymerase (Queen et al. (1989) Proc. Natl. Acad. Sci. USA 86:10029-10033) can be used under the invention to provide molecules having altered or enhanced antigen-binding capabilities, and/or reduced immunogenicity.

Once coding sequences have been prepared or isolated, such sequences can be cloned into any suitable vector or replicon. Numerous cloning vectors are known to those of skill in the art, and the selection of an appropriate cloning vector is a matter of choice. Suitable vectors include, but are not limited to, plasmids, phages, transposons, cosmids, chromosomes (including artificial chromosomes, such as BACs or YACs) or viruses which are capable of replication when associated with the proper control elements.

The coding sequence is then placed under the control of suitable control elements, depending on the system to be used for expression. Thus, the coding sequence can be placed under the control of a promoter, ribosome binding site (for bacterial expression) and, optionally, an operator, so that the DNA sequence of interest is transcribed into RNA by a suitable transformant. The coding sequence may or may not contain a signal peptide or leader sequence which can later be removed by the host in post-translational processing. See, e.g., U.S. Pat. Nos. 4,431,739; 4,425,437; 4,338,397.

In addition to control sequences, it may be desirable to add regulatory sequences which allow for regulation of the expression of the sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector. For example, enhancer elements may be used herein to increase expression levels of the constructs. Examples include the SV40 early gene enhancer (Dijkema et al. (1985) EMBO J 4:761), the enhancer/promoter derived from the long terminal repeat (LTR) of the Rous Sarcoma Virus (Gorman et al. (1982) Proc. Natl. Acad. Sci. USA 79:6777) and elements derived from human CMV (Boshart et al. (1985) Cell 41:521), such as elements included in the CMV intron A sequence (U.S. Pat. No. 5,688,688). The expression cassette may further include an origin of replication for autonomous replication in a suitable host cell, one or more selectable markers, one or more restriction sites, a potential for high copy number and a strong promoter.

An expression vector is constructed so that the particular coding sequence is located in the vector with the appropriate regulatory sequences, the positioning and orientation of the coding sequence with respect to the control sequences being such that the coding sequence is transcribed under the “control” of the control sequences (i.e., RNA polymerase which binds to the DNA molecule at the control sequences transcribes the coding sequence). Modification of the sequences encoding the molecule of interest may be desirable to achieve this end. For example, in some cases it may be necessary to modify the sequence so that it can be attached to the control sequences in the appropriate orientation; i.e., to maintain the reading frame. The control sequences and other regulatory sequences may be ligated to the coding sequence prior to insertion into a vector. Alternatively, the coding sequence can be cloned directly into an expression vector which already contains the control sequences and an appropriate restriction site.

As explained above, it may also be desirable to produce mutants or analogs of the antigen of interest. Methods for doing so are described in, e.g., Dasmahapatra et al., U.S. Pat. No. 5,843,752 and Zhang et al., U.S. Pat. No. 5,990,276. Mutants or analogs of SARSV proteins for use in the subject assays may be prepared by the deletion of a portion of the sequence encoding the polypeptide of interest, by insertion of a sequence, and/or by substitution of one or more nucleotides within the sequence. Techniques for modifying nucleotide sequences, such as site-directed mutagenesis, and the like, are well known to those skilled in the art. See, e.g., Sambrook et al., supra; Kunkel, T. A. (1985) Proc. Natl. Acad. Sci. USA (1985) 82:448; Geisselsoder et al. (1987) BioTechniques 5:786; Zoller & Smith (1983) Methods Enzymol. 100:468; Dalbie-McFarland et al. (1982) Proc. Natl. Acad. Sci USA 79:6409.

The molecules can be expressed in a wide variety of systems, including insect, mammalian, bacterial, viral and yeast expression systems, all well known in the art.

For example, insect cell expression systems, such as baculovirus systems, are known to those of skill in the art and described in, e.g., Summers & Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells, human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa califormica, Bombyx mori, Drosophila melanogaster, Spodoptera frugiperda, and Trichoplusia ni.

Nucleic acid molecules comprising nucleotide sequences of interest can be stably integrated into a host cell genome or maintained on a stable episomal element in a suitable host cell using various gene delivery techniques well known in the art. See, e.g., U.S. Pat. No. 5,399,346.

Depending on the expression system and host selected, the molecules are produced by growing host cells transformed by an expression vector described above under conditions whereby the protein is expressed. The expressed protein is then isolated from the host cells and purified. If the expression system secretes the protein into growth media, the product can be purified directly from the media. If it is not secreted, it can be isolated from cell lysates. The selection of the appropriate growth conditions and recovery methods are within the skill of the art.

### EXAMPLE

For useful expression of SARSV antigens in Saccharomyces cerevisiae and Pichia pastoris, insect cells, and mammalian cells, the following domains are cloned into expression vectors as listed in the Table below. The nt sequence numbers are from the SARSV sequence of SEQ ID NO: 1.
RNA polymerase 1a: SARS nt 250-13398
RNA polymerase 1b: SARS nt 13399-21470
ORFns.envelope (homologous to ns2, hemagglutinin-esterase envelope glycoprotein, and spike glycoprotein): SARS nt 21477-25244
Membrane: SARS nt 27849-28103
Nucleocapsid: SARS nt 28105-29373

A combination of PCR and synthetic oligos is used to create the above domains with restriction sites tailored to the following expression vectors:

| Restriction ends | Vector | Promoter | Expression host |
|---|---|---|---|
| HindIII/SalI | pBS24.1 | ADH2/GAPDH | AD3/Saccharomyces |
| EcoRI/Sal\\\ | pBS24.1 | ADH2/GAPDH/SOD fusion | AD3/Saccharomyces |
| XbaI/SalI | pAO815 | AOXI | GS115/Pichia pastoris |
| | | | HVK-293/Transient |
| EcoRI/BamHI | pCMVkm2 | CMVp/Enhancer/IntronA | transfection |
| EcoRI/XmaI | pCMVIII | CMVp/Enhancer/IntronA | CHO stable cell line |
| | | | Cell lines employed by Chiron |
| NheI/SalI | pBluBac4.5 | Polyhedrin | include: Sf9, Sf21, Tn5 |

IV. Treatment of SARS Infection With RNAi

RNA interference or “RNAi” is a term initially coined by Fire and co-workers to describe the observation that double-stranded RNA (dsRNA) can block gene expression when it is introduced into worms (Fire et al., Nature 391, 806-811(1998)). RNAi most likely involves mRNA degradation, resulting in sequence-specific, post-transcriptional gene silencing in many organisms. RNAi is a post-transcriptional process triggered by the introduction of double-stranded RNA which leads to gene silencing in a sequence-specific manner. RNAi has been reported to occur naturally in organisms as diverse as nematodes, trypanosmes, plants and fungi. It most likely serves to protect organisms from viruses, modulate transposon activity and eliminate aberrant transcription products.

The first evidence that dsRNA could achieve efficient gene silencing through RNAi came from studies on the nematode Caenorhabditis elegans (Fire et al. (1998) Nature, 391:806-811 and U.S. Pat. No. 6,506,559). Later studies in the fruit fly Drosophila melanogaster demonstrated that RNAi is a two-step mechanism (Elbashir et al. (2001) Genes Dev., 15(2): 188-200). First, long dsRNAs are cleaved by an enzyme known as Dicer in 21-23 nucleotides (nt) fragments, called small interfering RNAs (siRNAs). Then, siRNAs associate with a ribonuclease complex (termed RISC for RNA Induced Silencing Complex) which target this complex to complementary mRNAs. RISC then cleaves the targeted mRNAs opposite the complementary siRNA, which makes the mRNA susceptible to other RNA degradation pathways.

RNAi is the phenomenon where dsRNA corresponding to a targeted DNA or RNA sequence can suppress or silence gene expression. Even though dsRNA can mediate gene-specific interference in mammalian cells in some circumstances (Wianny & Zernicka-Goetz (2000) Nature Cell Biol. 2:70-75; Svoboda et al. (2000) Development 17:4147-4156) the use of RNAi in mammalian somatic cells is often limited due to the dsRNA triggering dsRNA-dependent protein kinase (PKR) which in turn inactivates translation factor eIF2a and causes a generalized suppression of protein synthesis and often times apoptosis (Gil & Esteban (2000) Apoptosis 5:107-114).

Recently, gene-specific suppression using siRNA of approximately 21 or 22 base pairs in length, corresponding to targeted RNA or DNA sequences, were shown to disrupt the expression of these targeted sequences in mammalian cells (Elbashir, S. M., et al., Nature 411: 494-498 (2001)). However, it is not clear that all RNA or DNA sequences of a mammalian cell's genome are susceptible to siRNA. It is also uncertain that every mammalian cell type possesses the necessary machinery for effecting gene-specific suppression using siRNA. Further, siRNA is of limited use for at least two reasons: the transient nature of the suppression effect seen in cells where the siRNA has been administered; and in some instances the necessity for chemical synthesis of siRNAs before their use (Tuschl T., Nature Biotechnol., 20: 446-448 (2002)). Also the instability of these short, synthetic RNAs makes it presents problems for any long term use of these siRNAs a pharmaceutical.

To overcome this limitation, the present invention provides a modified siRNA with increased stability against nuclease degradation while still maintaining its ability to inhibit viral replication via RNA interference. Such modification to the ribonucleotides in the siRNAs, adds a chemical group via chemical synthesis or in vitro transcription or longer modified RNAs can be prepared by either of these methods and cut into siRNAs using Dicer.

Although other methods for gene-specific suppression have utilized chemically-modified nucleic acids, such as antisense and ribozyme technology, such modification destroys critical enzymatic activities necessary for the function of these technologies. In regard to antisense technology, modification of the ribonucleotides destroys RNaseH activity, whereas such modification abolishes the catalytic activity of ribozymes.

The present invention provides a double-stranded RNA (dsRNA) molecule modified for protection against nuclease degradation with a length from about 10 to about 30 nucleotides which is able to inactivate a virus in a mammalian cell. The invention also provides a method of inactivating a virus by administering modified small interfering RNAs (siRNAs) that are modified so that they are nuclease or RNase resistant and retain the biological activity of being able to inhibit viral replication by targeting a RNA sequence in a virus.

The invention is further directed to a method of making modified siRNAs that target a RNA sequence in a virus comprising preparing a modified-double stranded RNA (dsRNA) fragment containing at least one modified ribonucleotide in at least one strand that spans the genome of the virus; and cleaving the modified-dsRNA fragments with recombinant human Dicer resulting in more than one modified siRNA.

The present invention provides a modified dsRNA molecule of from about 10 to about 30 nucleotides which mediates targeted RNA interference in hepatic or SARS-infected cells.

As used herein RNA interference, or RNAi, is used to mean sequence-specific, or gene specific, suppression of gene expression (protein synthesis), without causing a generalized suppression of protein synthesis in cells harboring the siRNA. The invention is not limited to a particular theory of the mechanism of action of RNAi. For example, RNAi may involve degradation of messenger RNA (mRNA) in an RNA-induced silencing complex (RISC), preventing translation of the transcribed mRNA, or it may involve the methylation of genomic DNA, shunting transcription of the gene. The lack of gene expression caused by RNAi may be transient, lasting a short period of time, or it may be stable, or permanent, lasting an indefinite period of time.

The term RNA is meant as is recognized in the art. Further, as used herein, RNA is used to mean double-stranded RNA (dsRNA) or single-stranded RNA (ssRNA) or a dsRNA with a single-stranded overhang. dsRNAs-within the meaning of the present invention includes short interfering RNA (siRNA), micro RNA (miRNA) and small hairpin RNA (shRNA), Additionally, RNA is also used to mean messenger RNA (mRNA), transfer RNA (tRNA) or ribosomal RNA (rRNA).

The present invention is directed to small interfering RNA (siRNA) which have been chemically modified to confer increased stability against nuclease degradation yet these siRNAs are still able to bind to target RNAs, that may be present in a cells. In the case where the target RNA is a virus specific RNA, the modified siRNAs are able to bind to the virus specific RNAs and inactivate the virus. A modified siRNA of the present invention comprises a modified ribonucleotide, wherein the siRNA is resistant to enzymatic degradation, such as RNase degradation, and yet retains the ability to inhibit viral replication. The modified siRNA is more specifically modified at the 2′ position of the ribose in the siRNA. The modification is at the 2′ position of at least one ribonucleotide of said siRNA. Attachment of receptor-binding ligands to siRNA molecules can be used to target the siRNA to a desired cell type. For example, attachment of cholesterol at the 5′-end or 3′-end of the siRNA molecule, to give a cholesteryl siRNA, can enhance targeting to hepatocytes. Other ligands for receptor mediated siRNA targeting to liver include HBV surface antigen, LDL, and others.

More specifically, the siRNA is modified at at least one pyrimidine, at least one purine or a combination thereof. However, generally all pyrimidines, or all purines or a combination of all pyrimidines and all purines of the siRNA are modified. More preferably, the pyrimidines are modified and these pyrimidines are cytosine, a derivative of cytosine, uracil, a derivative of uracil or a combination thereof. It also is contemplated to modify the selected ribonucleotides in at least one strand of the siRNA or the ribonucleotides in both strands of the siRNA are modified.

The nucleotides containing pyrimidine bases found in RNA (cytidine and uridine) can be chemically modified by adding any molecule that inhibits RNA degradation or breakdown to the 2′ position of the ribose molecule. The 2′-modified pyrimidine nucleotide can be formed using a number of different methods. The 2′ modification confers increased stability to the siRNA by making the siRNA impervious or resistant to nuclease activity. Thus, the 2′ modified siRNA has a longer serum half-life and is resistant to degradation compared to unmodified siRNA. The siRNA also may be modified completely or partially.

Regarding chemical modification of siRNAs, a molecule from the halide chemical group is preferably added to the ribonucleotide of the siRNA. Within the halides, fluorine is the preferred molecule but other chemical molecules, in addition to fluoro-, such as methyl-, methoxyethyl- and propyl-modifications can also we made. But the preferred modications is fluoro-modification, such as a 2′-fluoro-modication or a 2′,2′-fluoro-modification. Thus, in a preferred embodiment of the invention, the siRNA is modified by adding a fluorine molecule to the 2′ carbon of the pyrimidine ribonucleotide. The siRNA may be fluorinated completely or partially. For example, only the cytosine nucleotides need be fluorinated. Alternatively, only the uracil nucleotide need be fluorinated but both uracil and cytosine can be fluorinated. Furthermore, only one strand, either sense or antisense, of the siRNA can be fluorinated. Even partial 2′ fluorination the siRNA gives protection against nucleolytic degradation. Furthermore, it is important to note the 2′ fluorinated siRNA is not toxic to cells, an unexpected result given that fluorine chemistry usually is toxic to living organisms.

The siRNA of the present invention is designed to interact with a target nucleotide sequence. Most preferably this target nucleotide sequence is a disease producing agent or pathogen of which one wishes to inhibit gene expression. More preferably, this target nucleotide sequence is in a virus genome, and further this virus genome is from a RNA virus or a DNA virus is selected from the group consisting of hepatitis C virus (HCV), hepatitis A virus, hepatitis B virus, hepatitis D virus, hepatitis E virus, Ebola virus, influenza virus, rotavirus, reovirus, retrovirus, poliovirus, human papilloma virus (HPV), metapneumovirus and coronavirus. The most preferred virus is SARS virus.

Modfied siRNA may be prepared in a number of ways, such as by chemical synthesis, T7 polymerase transcription, or by treating modified long double stranded RNA (dsRNA) prepared by one of the two previous methods with Dicer enzyme. Dicer enzyme can be used to cleave dsRNA that is about 500 base pairs to about 1000 base pairs in size, to created mixed populations of dsRNA from about 21 to about 23 base pairs in length. Furthermore, an unexpected result of using the Dicer enzyme method is that Dicer enzyme will cleave modified strands of dsRNA, such as 2′ fluorinated modified dsRNA. Before development of this method, it was previously thought that Dicer would not be able to cleave modified siRNA. The Dicer method can be carried out using the Dicer siRNA Generation Kit available from Gene Therapy Systems, San Diego, Calif.

As used herein, small interfering RNA (siRNA) is defined as double- or single-stranded RNA of from about 10 to about 30 nucleotides in length, more preferably 12-28 nucleotides, more preferably 15-25 nucleotides, even more preferably 19-23 nucleotides and most preferably 21-23 nucleotides. The length of a siRNA as used herein, is determined by the length of one of the strands of the RNA. For example, a siRNA that is described as 21 nucleotides long (a 21-mer) may comprise two opposite strands of RNA which anneal together for 19 contiguous base pairings. The two remaining nucleotides on one end of the molecule would not anneal to the opposite strand, thus creating an “overhang”. The overhang can be at the 5′ or the 3′ end of the dsRNA. Preferably, the overhang is at the 3′ end of the RNA strand. The length of a double-stranded RNA where the two opposite strands are not the same length will be designated by the longer of the two strands. For example, a dsRNA comprising one strand which is 21 nucleotides long and anneals to an opposite strand that is 20 nucleotides long, will be considered, as used herein, a 21-mer.

Preferably, the siRNA of the present invention will comprise a 3′ overhang of about 2 to 4 bases. More preferably, the 3′ overhang is 2 nucleotides long. Even more preferably, the 2 nucleotides comprising the 3′ overhang are uridine (U).

In one embodiment, the invention provides an RNA molecule comprising a nucleotide sequence at least 80% identical to the nucleotide sequence of the target agent or virus. Preferably, the RNA molecule of the present invention is at least 90%, 95%, 96%, 97%, 98%, 99% or 100% identical to the nucleotide sequence of the target agent or virus.

As a practical matter, whether any particular nucleic acid molecule is at least 90%, 95%, 96%, 97% 98%, 99% or 100% identical to the nucleotide sequence of the target agent or virus can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith & Waterman (Advances in Applied Mathematics 2:482-489 (1981)) to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present invention provides a method of inactivating a target agent or preferably a virus in a patient comprising administering to the patient a modified siRNA in an effective amount to inactivate the targeted agent or virus. RNA interference towards a targeted DNA segment in a cell can be achieved by administering a dsRNA molecule or siRNA to the cells, wherein the nucleotide sequence of the dsRNA molecule corresponds to the nucleotide sequence of the targeted DNA segment. Preferably, the RNA molecule used to induce targeted RNAi is siRNA.

Gene suppression, targeted suppression, sequence-specific suppression, targeted RNAi or sequence-specific RNAi are used interchangeably herein. Furthermore, sequence-specific suppression, as used herein, is determined by separately assaying the levels of the protein targeted for suppression in cells containing the siRNA (experimental cells) and in cells not containing the identical siRNA (control cells), and comparing the two values. Furthermore, the experimental and control cells must be derived from the same source and same animal. For example, the control and experimental cells can be, but are not limited to, normal human hepatic cells as cell culture in vitro, or they can derived from a hepatocellular carcinoma. Further, the control and experimental cells used in determining the level or quantity of gene suppression must be assayed under similar, if not identical, conditions.

As used herein the phrase “targeted DNA segment” is used to mean a DNA sequence encoding, in whole or in part, an mRNA for a targeted protein, including introns or exons, where suppression is desired. DNA segment can also mean a DNA sequence that normally regulates expression of the targeted protein, including but not limited to the promoter of the targeted protein. Furthermore, the DNA segment may or may not be a part of the cell's genome or it may be extrachromosomal, such as plasmid DNA.

The present invention is further directed to inactivating a virus in a patient comprising administering to a patient a modified siRNA in an effective amount to inactivate the virus. The siRNA is preferably about 10 to about 30 nucleotides in length, more preferably 12-28 nucleotides, more preferably 15-25 nucleotides, even more preferably 19-23 nucleotides and most preferably 21-23 nucleotides. The method preferably utilizes a 2′ modified siRNA that is modified at the 2′ position of at least one ribonucleotide of said siRNA. The method utilizes a siRNA that is modified with chemical groups selected from the group consisting of fluoro-, methyl-, methoxyethyl- and propyl-modification. The fluoro-modification is preferred and either a 2′-fluoro-modication or a 2′,2′-fluoro-modification is useful in the present invention and preferred.

The modification may be at the pyrimidines, the purines or a combination thereof of the siRNA are modified. More preferably the pyrimidines are modified, such as cytosine, a derivative of cytosine, uracil, a derivative of uracil or a combination thereof. In one embodiment, at least one strand of the siRNA contains at least one modified nucleotide and in an alternate embodiment, oth strands of the siRNA contains at least one modified nucleotide.

The method is intended to target disease causing agents or pathogens, an more particularly viruses, which can be either a RNA virus or a DNA virus, which are selected from the group consisting of hepatitis C virus (HCV), hepatitis A virus, hepatitis B virus, hepatitis D virus, hepatitis E virus, Ebola virus, influenza virus, rotavirus, reovirus, retrovirus, poliovirus, human papilloma virus (HPV), metapneumovirus and coronavirus. More preferably the target virus is a SARS virus. The present method utilizes a siRNA prepared by (a) identifying a target nucleotide sequence in a virus genome, preferably SARS virus, for designing a small interfering RNA (siRNA); and (b) producing a siRNA that has been modified to contain at least one modified nucleotide. More preferably, the siRNA comprises a dsRNA molecule with a first strand ribonucleotide sequence corresponding to a nucleotide sequence corresponding to a target nucleotide sequence in said virus and a second strand comprising a ribonucleotide sequence complementary to said target nucleotide sequence, wherein said first and second strands are separate complementary strands that hybridize to each other to form said dsRNA molecule, and further wherein the first strand ribonucleotide sequence, the second strand ribonucleotide sequence or both the first and second strand ribonucletide sequences comprise at least one modified nucleotide. In this method, the target nucleotide sequence comprises a conserved nucleotide sequence necessary for SARS virus replication, and the conserved nucleotide sequence is selected from the group consisting of SEQ ID NO: 7292, SEQ ID NO: 7293, SEQ ID NO: 7294, SEQ ID NO: 7295, SEQ ID NO: 7296, SEQ ID NO: 7297, SEQ ID NO: 7298, SEQ ID NO: 7299, SEQ ID NO: 7300 and SEQ ID NO: 7301. Preferably, the nucleotide sequence is selected from the group consisting of SEQ ID NO: 7292 and SEQ ID NO: 7293. Still more preferably, the nucleotide sequence is SEQ ID NO: 7293.

The siRNA disclosed in this application may be prepared with modified ribonucleotides as described herein. Further, the modified ribonucleotide of the siRNA used in the present method is incorporated into said siRNA by chemical synthesis or enzymatic synthesis.

The siRNA disclosed in this application may or may not have a 5′ triphosphate group.

The modified siRNA is administered to a patient by a method selected from the group consisting of intravenous injection, subcutaneous injection, oral delivery, and liposome delivery. The modified siRNA accumulates in an organ, tissue or body system of the patient that are the liver, gastrointestinal tract, respiratory tract, cervix or skin.

The present invention also provides a method of inhibiting the replication of a virus, such as SARS virus, in cells positive for SARS virus comprising transfecting SARS-positive cells with a vector that directs the expression of modified siRNA which is specific for SARS. The cells are evaluated to determine if a marker in the cells has been inhibited by the modified siRNA.

The term patient, as used herein, can be an animal, preferably a mammal. More preferably the subject can be a primate, including non-human and humans. The terms subject and patient can be used interchangeably.

The treatment envisioned by the current invention can be used for subjects with a pre-existing viral infection, or for subjects pre-disposed to a SARS virus infection. Additionally, the method of the current invention can be used to correct or compensate for cellular or physiological abnormalities involved in conferring susceptibility to viral infections in patients, and/or to alleviate symptoms of a viral infection in patients, or as a preventative measure in patients.

The method of treating a patient having a viral infection involves administration of compositions to the subjects. As used herein, composition can mean a pure compound, agent or substance or a mixture of two or more compounds, agents or substances. As used herein, the term agent, substance or compound is intended to mean a protein, nucleic acid, carbohydrate, lipid, polymer or a small molecule, such as a drug.

In one embodiment of the current invention, the composition administered to the subject is a pharmaceutical composition. Further, the pharmaceutical composition can be administered orally, nasally, parenterally, intrasystemically, intraperitoneally, topically (as by drops or transdermal patch), bucally, or as an oral or nasal spray. The term “parenteral,” as used herein, refers to modes of administration which include intravenous, intramuscular, intraperitoneal, intrasternal, subcutaneous and intraarticular injection and infusion. The pharmaceutical compositions as contemplated by the current invention may also include a pharmaceutically acceptable carrier.

By “pharmaceutically acceptable carrier” is intended, but not limited to, a non-toxic solid, semisolid or liquid filler, diluent, encapsulating material or formulation auxiliary of any type, such as liposomes.

A pharmaceutical composition of the present invention for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention can also contain adjuvants such as, but not limited to, preservatives, wetting agents, emulsifying agents, and dispersing agents. Prevention of the action of microorganisms can be ensured by the inclusion of various antibacterial and antifungal agents, for example, paraben, chlorobutanol, phenol, sorbic acid, and the like. It can also be desirable to include isotonic agents such as sugars, sodium chloride, and the like. Prolonged absorption of the injectable pharmaceutical form can be brought about by the inclusion of agents which delay absorption such as aluminum monostearate and gelatin.

In some cases, to prolong the effect of the drugs, it is desirable to slow the absorption from subcutaneous or intramuscular injection. This can be accomplished by the use of a liquid suspension of crystalline or amorphous material with poor water solubility. The rate of absorption of the drug then depends upon its rate of dissolution which, in turn, can depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include, but are not limited to, capsules, tablets, pills, powders, and granules. In such solid dosage forms, the active compounds are mixed with at least one item pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, acetyl alcohol and glycerol monostearate, h) absorbents such as kaolin and bentonite clay, and i) lubricants such as talc, calcium stearate, magnesium stearate, solid polyethylene glycols, sodium lauryl -sulfate, and mixtures thereof. In the case of capsules, tablets and pills, the dosage form can also comprise buffering agents.

Solid compositions of a similar type can also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They can optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include, but are not limited to, pharmaceutically acceptable emulsions, solutions, suspensions, syrups and elixirs. In addition to the active compounds, the liquid dosage forms can contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, can contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Alternatively, the composition can be pressurized and contain a compressed gas, such as nitrogen or a liquefied gas propellant. The liquefied propellant medium and indeed the total composition is preferably such that the active ingredients do not dissolve therein to any substantial extent. The pressurized composition can also contain a surface active agent. The surface active agent can be a liquid or solid non-ionic surface active agent or can be a solid anionic surface active agent. It is preferred to use the solid anionic surface active agent in the form of a sodium salt.

The compositions of the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the compounds of the invention, stabilizers, preservatives, excipients, and the like. The preferred lipids are the phospholipids and the phosphatidyl cholines (lecithins), both natural and synthetic. Methods to form liposomes are known in the art (see, for example, Prescott, Ed., Meth. Cell Biol. 14:33 et seq (1976)).

One of ordinary skill will appreciate that effective amounts of the agents of the invention can be determined empirically and can be employed in pure form or, where such forms exist, in pharmaceutically acceptable salt, ester or prodrug form. The agents can be administered to a subject, in need of treatment of viral infection, as pharmaceutical compositions in combination with one or more pharmaceutically acceptable excipients. It will be understood that, when administered to a human patient, the total daily usage of the agents or composition of the present invention will be decided by the attending physician within the scope of sound medical judgement. The specific therapeutically effective dose level for any particular patient will depend upon a variety of factors: the type and degree of the cellular or physiological response to be achieved; activity of the specific agent or composition employed; the specific agents or composition employed; the age, body weight, general health, sex and diet of the patient; the time of administration, route of administration, and rate of excretion of the agent; the duration of the treatment; drugs used in combination or coincidental with the specific agent; and like factors well known in the medical arts. For example, it is well within the skill of the art to start doses of the agents at levels lower than those required to achieve the desired therapeutic effect and to gradually increase the dosages until the desired effect is achieved.

Dosing can also be arranged in a patient specific manner to provide a predetermined concentration of the agents in the blood, as determined by techniques accepted and routine in the art. Thus patient dosaging can be adjusted to achieve regular on-going blood levels, as measured by HPLC, on the order of from 50 to 1000 ng/ml.

It will be readily apparent to one of ordinary skill in the relevant arts that other suitable modifications and adaptations to the methods and applications described herein can be made without departing from the scope of the invention or any embodiment thereof.

The modified siRNA is prepared by custom chemical synthesis by Dharmacon, at Lafayette Colo. Each C and U within the siRNA duplex (GL2), has been substituted with 2′-F-U and 2′-F-C except for the 3′-end overhang, which was dTdT.

To test the stability of 2′ chemically modified siRNA compared to unmodified siRNA (siRNA), the following experiment is performed. 4 ngs of siRNA are added to a 20 μL volume of 80% human serum from a healthy donor. This mixture is incubated at 37° C. for various times ranging from 1 minute up to 10 days. The same process is performed for 2′ fluorine modified siRNA (2′-F siRNA). When the incubation process is finished, the mixtures are placed on ice and then immediately separated by PAGE along with a ³²P-siRNA control. The 2′ modified siRNA is stable as compared to unmodified siRNA.

V. Identification of Therapeutically Active Agents for Treatment of SARS Virus Infection

The invention provides methods for treating SARS by administering therapeutically active agents, such as small molecule compounds, to a mammal, as well as methods of identifying therapeutically active agents, such as potent small molecules, for the treatment of SARS virus infection.

In one aspect of the invention a method of identifying a therapeutically active agent is provided comprising: (a) contacting the therapeutically active agent with a cell infected with the SARS virus; (b) measuring attenuation of a SARS related enzyme.

In a more particular embodiment, the therapeutically active agent is a small molecule. In another more particular embodiment, the therapeutically active agent is a nucleoside analog (e.g. Ribavirin). In another more particular embodiment the small molecule is a SMIP or peptidic immunomodulating compound. In another more particular embodiment the therapeutically active agent is a peptoid, oligopeptide, or polypeptide. In another embodiment the SARS related enzyme is SARS protease. In another embodiment the SARS related enzyme is SARS polymerase. In still another embodiment the SARS related enzyme is a kinase. In still another embodiment, the SARS related enzyme is a protease. The furin inhibitor peptidyl chloromethylketone prevents blocks cell-cell fusion after MHV infection (de Haan et al. (2004) J Virol), which offers guidance for SARS therapy.

The invention includes a cell-based assay that can be used to screen for and identify a therapeutically active agent for the treatment of SARS virus infection. Therapeutically active agents of the invention include agents that inhibit, prevent or reduce the replication of a SARS virus. Such agents can be identified by infecting a cultured cell (such as, for example, VERO cells) with a SARS virus and evaluating the impact of potential antiviral compounds on SARS virus replication. Assays to measure the effect of a potential antiviral compound on virus replication are known in the art and may be based on a variety of parameters.

The cell-based assay may be used in a high-throughput screen to identify therapeutically active compounds from chemical libraries comprising potential antiviral compounds. Therapeutically active compounds suitable for use in the invention may inhibit any SARS viral target that is essential for viral replication in whole cells. Efficacy (the ability of a compound to inhibit or inactivate the target, be it viral or cellular, that results in the reduction of virus in the culture) of the therapeutic agent is measured by assessing the viability and/or the proliferation of surviving cells in a SARS virus infected cell culture.

A number of methods can be used to measure cell viability are known in the art, such as assays measuring cellular enzymes, proteins, nucleotide triphoshates (e.g. ATP), nucleic acids (e.g. host cell mRNA (e.g. GAPDH) or rRNA sequences) or cellular metabolites such as MTT or MTS. In addition, fluorescent (including, for example HSV paper) or non-fluorescent dyes (e.g. propidium diiodide) or labeling of DNA can be used to measure indications of cell viability and/or proliferation.

Alternatively, efficacy of a compound or sample can be determined by directly measuring the amount of virus or viral products in the culture. Methods for measuring the amount of virus, viral genome or viral products include: PCR, RT-PCR, TMA, reporter proteins with fluourescent or luminescent qualities or enzymatic functions (e.g., luciferase, alkaline phosphatase, GFP) or proteins that can be detected by antibodies (e.g. EGF) that might be incorporated into the viral genome prior to infection of the cell culture. Further, viral products such as viral proteins can be measured by ELISA or enzymatic activities. Methods for identifying viral polynucleotides, viral proteins and antibodies specific to viral proteins are discussed above.

Potential antiviral compounds are applied to the cell-based assay at a concentration of approximately 10 μM and compound classes having therapeutic effect are identified by measuring the parameter of choice (such as cell viability/proliferation or the virus or viral genome or a viral product be it viral in origin or non-virus in orgin). Once compounds are identified as having activity, they are resynthesized, and analoged. Starting with the identified compound, many analogs and new compounds are synthesized during consecutive optimization cycles of synthesis, biological profiling and modeling techniques to optimize the to the lead structure until in vivo activity is elucidated and optimized.

Cells suitable for use in the assay include the cells described above as suitable for vaccine production. Preferably, the cells are African green monkey kidney cells (Vero) cells. Human embyronic lung fibroblasts or normal human diploid fibroblasts may also be used in the invention.

In one embodiment, the invention includes a fluorescence based cytopathogenicity assay to measure the effect of a potential antiviral compound on a cell-based assay. One example of a fluorescence based cytopathogenicity assay is illustrated below.

1×10⁴ Vero cells per well of a microtiter plate (MTP) are infected with a defined amount of SARS virus selected within the following ranges for optimal MOI: 5-10, 10-25, 25-50, 50-100, 100-500, or 500-1000 PFU in a total volume of 200 μl media (M199 medium supplemented with 5% FCS, 2 mM glutamine, 100 IU/ml penicillin and 100 μg/ml streptomycin) in the presence or absence of the potential antiviral compound and incubated for at least 1, 2, 3, 4, 5, 6, or 7 days at 37° C., 5% CO₂. The wells of the MTP are washed with PBS (200 μl) and then filled with 200 μl PBS containing 10 μg/ml fluorescein diacetate. After a 45-min incubation at room temperature, fluorescence is measured at 485 nm excitation and 538 nm emission wavelengths. IC₅₀ values are determined by a nonlinear plot of antiviral activity as a function of drug concentration.

Other cell based assays are known in the art and include, among others, methods of GFP detection and Luc detection. In addition, a Promega kit is commercially available that provides additional methods of measuring cell viability, etc.

In one embodiment, the invention includes a method of measuring the efficacy of a potential antiviral compound using RT-PCR to detect the levels of SARS viral RNA in the cell based assay. Methods of using RT-PCR are known in the art. One example of such an assay is described below.

5×10⁶ Vero cells are seeded in tissue culture. Flasks containing the cells are incubated over night at 37° C., 5% CO₂. The cells are infected (m.o.i.=1) with SARS virus in the presence and absence of potential antiviral compounds. Optionally, the cells may be pretreated with the potential compound prior to infection. In either case, a suitable control cell assay is also prepared.

The RNA of infected cells is purified at 2 h (UL54), 12 h (UL8) and 16 h (UL13) after infection, (Qiagen) RNA purification (RNeasy kit; 40 μl elution) and quantified (absorption at 260 nm). The RNA (2 μg) is reverse transcribed with a specific primer (2 pmol, using one of the primer pairs described herein) into cDNA according to the Superscript II protocol (Invitrogen). Aliquots (2 μl) of the reverse transcription reaction are amplified by PCR. Fragments of the appropriate target SARS gene, i.e., a gene encoding a SARS enzyme, are amplified in 30 cycles (UL54 and UL8: 3 min, 94° C. hot start; 1 min, 94° C. denaturation; 1 min, 55° C. annealing; 1 min, 72° C. polymerization. UL13: 3 min, 94° C. hot start; 1 min, 94° C. denaturation; 1 min, 60° C. annealing; 1 min, 72° C. polymerization) by PCR (Taq-Polymerase, Stratagene), in a 100-μl reaction volume with the appropriate oligonucleotides, as described herein at 0.1 nmol each. 8-μl aliquots of cycle 20-30 (lanes 2-12) of the PCR were resolved on a 2% agarose gel (Invitrogen) according to the manufacturer's instructions.

Cell-based assays of the invention may optionally use a variant or derivative of a wild-type SARS virus that has reduced or attenuated virulence in humans and/or animal models (e.g., mouse, non-human primate, etc.) Use of such attenuated SARS viruses in screening methods may reduce safety concerns and precautions that would otherwise be associated with the pathogenic nature of the SARS virus and may eliminate or reduce the need for the implementation of cumbersome high containment levels during performance of the assays and screening of compounds.

The invention includes an enzyme-based assay that can be used to screen for and identify a therapeutically active agent for the treatment of SARS virus infection.

An embodiment of the invention is an assay comprising contacting a known quantity of SARS protease in solution to a peptide containing a detectable marker and cleavage site for SARS protease, wherein SARS protease activity is monitored by measuring the intensity of the marker on the cleaved product.

In a more particular embodiment, a method of assaying for SARS protease is provided comprising contacting a sample solution containing SARS protease with a peptide containing a fluorescent donor, fluorescent quencher, and cleavage site for SARS protease, said peptide being detectable with a fluorometer when cleaved, wherein SARS protease activity is determined in the sample by the amount of fluorescence detected by the fluorometer.

Assays based on the direct measurement of SARS protease inhibition may be utilized for screening for SARS therapeutics. Protease for such assays such as 3C-like protease and papain-like protease may be isolated and purified for such assays as described in Seybert, et al., J. Gen. Virol., 78:71-75, 1997, Ziebuhr, et al., Adv. Exp. Med. Biol., 440:115-120, 1998, Sims, et al., Adv. Exp. Med. Biol. 440:129-134, 1998, Ziebuhr, et al., J. Virol., 73:177-185, 1999, Teng, et al., J. Virol., 73:2658-2666, 1999, Herold, et al., J. Biol. Chem. 274:14918-14925, 1999, and Ziebuhr, et al., J. Biol. Chem. 276:33220-33232, 2001. Furthermore, Example 30 describes a novel method of purifying SARS protease using column chromatography. Example 31 describes a continuous fluorescence resonance energy transfer (FRET) assay for measuring SARS protease activity. Protease enzyme based assays such as the FRET assay demonstrated in Example 31 are readily adapted for high-throughput screening and are used for screening candidate antiviral compounds. Performance of the protease enzymatic assay in the presence of a SARS protease inhibitor compound will show a decreased amount of fluorescence at a given time when compared to negative control assay containing no test compound on a non-inhibiting control compound. Such a method would involve the steps of: (a) providing an assay solution comprising SARS protease; (b) adding a test compound to the assay solution; (c) adding a substrate for SARS protease to the assay solution; and (d) measuring the proteolytic activity in the assay solution. In a preferred embodiment, the proteolytic activity is measured by the fluorescence of fluorophore product produced by the enzymatic activity of SARS protease.

Attenuated SARS virus variants generally contain one or more genome modifications or mutations (e.g., substitutions, deletions, insertions) in protein encoding or non-coding regions. Specific examples of attenuating mutations include, for example, genetic modifications in the 5′-end noncoding region, leader sequence, intergenic regions, 3′-end noncoding region, ORF 1a, ORF 1b, S gene, E gene, M gene, N gene, or any of the nonstructural protein genes outside of the ORF 1a/1b region. Preferred attenuating mutations are in a SARS virus structural protein (e.g., Spike (S)), a protease or polymerase domain, or a non-coding sequence (e.g., 5′-end noncoding region, intergenic sequence). In addition, a cleavage site may be introduced or eliminated within the spike protein (see for example, Gombold et al., J. Virol. 67:4504-4512, 1993; Bos et al., Virology 214:453-463, 1995), such modification that may also be useful for optimization of expression of recombinant spike protein antigen (e.g., for vaccine purposes).

A variety of methods are used according to the present invention in order to obtain attenuated variants of SARS virus. Such methods include serial passage of the SARS virus in cultured cells (e.g., mammalian cell culture, such as fetal rhesus kidney cells or VERO cells), until the SARS virus demonstrates attenuated function. The serial propagation of virus may be performed at any temperature at which tissue culture passage attenuation occurs, and may be performed in conjunction with one or more steps of mutagenesis (e.g., chemical mutagenesis). The attenuated phenotype of SARS virus variants, obtained after one or more cell culture passages, is readily measured by one skilled in the art. As used herein, attenuation refers to the decreased virulence of the SARS virus in a human subject. Evidence of attenuated function may be indicated by decreased levels of viral replication or by decreased virulence in an animal model.

Other methods of producing an attenuated SARS virus include cell culture passage of the virus at sub-optimal temperatures (cold passage), as well as introduction of attenuating mutations into the SARS viral genome by random mutagenesis (e.g., chemical mutagenesis, such as using 5-fluorouracil) or using directed mutagenesis. Preparation and generation of attenuated RSV vaccines (the methods of which will generally applicable to SARS virus) are disclosed in, for example, EP 0640128, U.S. Pat. No. 6,284,254, U.S. Pat. No. 5,922,326, U.S. Pat. No. 5,882,651.

The number of passages required to obtain safe, immunizing attenuated virus is dependent at least in part on the conditions employed. Periodic testing of the SARS virus culture for virulence and immunizing ability in animals (e.g., mouse, primate) can readily determine the parameters for a particular combination of tissue culture and temperature.

In another embodiment, the cell-based assay for screening of antiviral compounds is based on the readout of expression of a gene product (e.g., reporter gene product) that is not from SARS virus. Gene products particularly suitable to the present invention include, but are not limited to those of the above-described assays.

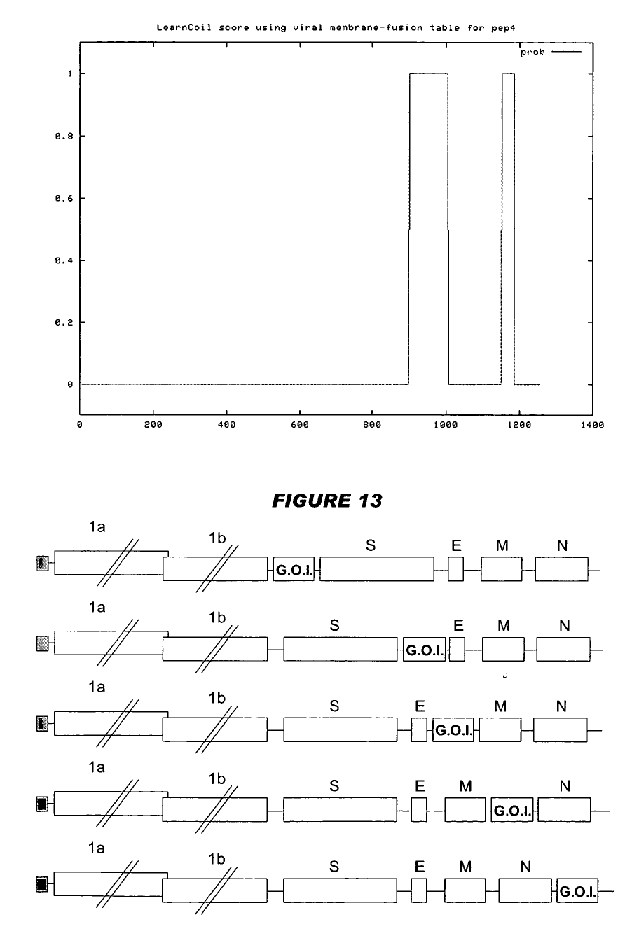

In order to achieve such a read-out, the gene-of-interest (GOI) encoding said gene reporter gene product must be incorporated into a replicating SARS virus genome or construct derived from a SARS virus genome (e.g., SARS virus replicon, SARS virus defective-interfering (DI) RNA). FIG. 13 is a schematic depicting locations for incorporation of the reporter gene into a SARS virus genome. Preferably, insertion of a heterologous reporter gene-of-interest is at a site between existing SARS virus genes, such as for example, as shown in FIG. 13. For example, the GOI may be inserted closely following the termination codon of a SARS virus gene (e.g., ORF 1b, S, E, M, N). Insertion should be positioned in order to minimize disruption of mRNA transcription for the SARS virus gene(s). The GOI may also be inserted as an in-frame “fusion” with an existing SARS virus gene, such that sufficient function of the GOI is maintained for detection. To optimize expression, an additional SARS virus intergenic sequence (e.g., SEQ ID NO: 7388, with or without additional flanking SARS virus sequences) may also be engineered into a position preceding the inserted GOI.

Incorporation of a GOI into SARS virus may be accomplished by one of skill in the art using a variety of techniques. For example, one preferred method is targeted RNA recombination, that takes advantage of the ability of coronavirus RNAs to undergo recombination within the cell (see for example Fischer et al., J. Virol. 71:5148-5160, 1997; Koljesar et al., J. Vet. Sci. 2:149-157, 2001). A construct of desired configuration (e.g., cDNA of defective interfering RNA of SARS virus) containing the GOI flanked by SARS virus sequence (e.g., intergenic sequence) is generated such that RNA may be transcribed directly within a eukaryotic cell or in vitro and transfected into susceptible cells also infected with SARS virus. Recombinant virus containing the GOI is identified based on expression of the GOI encoded marker.

Alternatively, incorporation of a GOI into SARS virus may be accomplished by one of skill in the art by first assembling a full-length cDNA clone of the SARS virus, that can be used to produce infectious RNA transcripts in vivo (e.g., from an RNA polymerase II promoter) or in vitro (e.g., from a bacteriophage promoter). Although relatively long in genome length, such assembly of a full-length cDNA clone is now readily obtainable by one of skill in the art using standard molecular biology and reverse genetics techniques and the genome sequence of SARS virus (see for example, Thiel et al., J. Gen. Virol., 82:1273-1281, 2001; Almazan et al., Proc. Natl. Acad. Sci. USA 97:5516-5521, 2000; Thiel et al. (2003) J Gen Virol 82:1273-1281; Yount et al (2003) PNAS USA 100:12995-13000). Insertion of a heterologous GOI into a full-length SARS virus genome cDNA may be performed using a variety of techniques, such as for example, ligation into natural or synthetic restriction sites, PCR (e.g., overlapping PCR), and recombination.

It may also be desirable to utilize similar SARS virus recombinants containing a gene-of-interest for antiviral screening, however, with further modification-to minimize or eliminate virus-induced cytopathology (e.g., CPE). Non-cytopathic derivatives from SARS virus may be obtained by one of skill in the art using a variety of methods. For example, a selectable marker (e.g., drug resistance marker) may be incorporated as GOI into a SARS virus genome to produce infectious virus as described above (see for example, Perri et al., J. Virol., 74:9802-9807, 2000). Infectious GOI-containing SARS virus or infectious genome RNA/cDNA is then used to infect/transfect cells (e.g., VERO), with or without prior mutagenesis, after which time the infected cells are subjected to the appropriate selection. Only those cells containing SARS virus harboring both the selectable marker and one or more mutations rendering the virus non-cytopathic will survive the selection process and grow out. Active SARS virus replication in these cells is readily detected using a variety of detection techniques (e.g., PCR, Northern blot) and such cells may serve as the substrate for cell-based screening assays. Mutations that result in the desired noncytopathic SARS virus phenotype may include nucleotide substitutions, deletions or additions, and may occur in a variety of genome coding or non-coding regions (e.g., 5′ or 3′-end noncoding regions, intergenic regions, ORF1a, ORF1b, a protease domain, a polymerase domain). The identification of such mutations is readily accomplished by exchange of sequences with wild-type (e.g., parental) SARS virus and demonstrating transfer of the phenotype, and sequencing of the appropriate genome region. Similar mutations that reduce or eliminate cytopathogenicity also may be utilized in the context of a SARS virus derived replicon vector, either by similar selection directly using a SARS virus replicon or by specific engineering of the replicon based on mutation(s) identified in the context of infectious SARS virus as described above. In addition, such mutations may serve as the basis for attenuated SARS virus derivatives, as described elsewhere in this document.

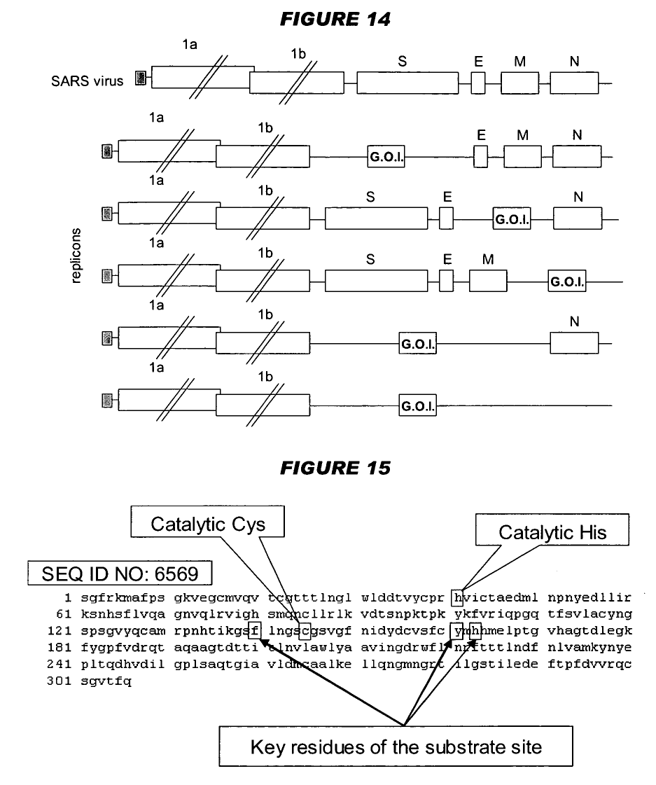

Alternatively, rather than using infectious SARS virus or its derivatives for cell-based screening assays, propagation defective “replicons” may be engineered and utilized. Such replicons maintain all protein encoding sequences and cis replication sequences required for RNA replication and expression within a cell, but are deleted of one or more sequences or genes required for packaging of progeny SARS virus (see for example Curtis et al., J. Virol., 76:1422-1434, 2002). FIG. 14 is a schematic depicting representative examples of SARS virus replicons according to the present invention. For example a SARS virus cDNA construct is generated, that is lacking one or more (or all) structural protein encoding genes, whereby the missing SARS virus gene(s) is/are replaced by the GOI, maintaining all necessary transcription signals for expression of the GOI. Operably linked with the SARS virus replicon cDNA construct is a promoter for RNA polymerase that can be used to transcribe the replicon RNA in vivo (e.g., RNA polymerase II promoter) or in vitro (e.g., bacteriophage promoter). The SARS replicon may be introduced into a susceptible cell by transfection as RNA or DNA, depending on the promoter of choice, and the transfected cells may be utilized for the evaluation of antiviral compounds. By incorporating one or more mutations rendering the replicon noncytopathic for the cells (see above), one can avoid the need for nucleic acid transfection each time an assay is to be performed.

Alternatively, SARS virus replicons may be packaged into virus like particles that allow infection of cells, rather than requiring transfection of nucleic acid molecules. A requirement for replicon packaging is that essential SARS virus gene functions deleted from the replicon (e.g., one or more structural proteins) are provided in trans within the cell containing the replicon. A variety of methods for packaging of replicon RNA can be utilized to one of skill in the art (see for example, Curtis et al., ibid: Ortego, et al., J. Virol., 76:11518-11529, 2002). For example, stably transformed cell lines constitutively or inducibly expressing the required SARS virus gene functions may be utilized. Alternatively, the required SARS virus gene functions may be expressed by viral vectors that are introduced into the replicon-containing cell. Alternatively a defective interfering (DI) SARS virus derived RNA containing the required gene functions may be introduced into the replicon-containing cell. Such DI constructs used to complement missing replicon functions may be more commonly referred to as defective helper RNA or defective helpers.

Another configuration useful for cell-based antiviral screening assays according to the present invention utilizes SARS virus derived DI RNAs encoding a GOI (see for example Stirrups, et al., J. Gen. Virol., 81:1687-1698, 2000; Liao, et al., Virology 208:319-327, 1995). Introduction of a SARS DI, either as cDNA linked to an RNA polymerase II promoter or as in vitro transcribed RNA, into susceptible cells also infected with SARS virus, allows for a readout of the GOI reporter product in assays.

A replicon-based system for rapid identification of coronavirus replicase inhibitors is described by Hertzig et al. (2004) J Gen Virol DOI 10.1099/vir/0/80044-0. Briefly, the system uses a non-cytopathic selectable replicon RNA that can be stably maintained in eukaryotic cells. The replicon RNA mediates reporter gene expression as a marker for coronavirus replication, and expression of the reporter can be used to test the inhibitory effect of test compounds in vitro, thereby allowing high throughput screening for replicase inhibitors without the need to grow infectious virus. Preferred replicon RNAs include a neomycin resistance gene in the replicase gene with a downstream reporter gene (e.g. GFP) that is expressed via replicase-mediated synthesis of a sub-genomic mRNA.

VI. Compositions and Methods for Treatment of SARS Virus Infection

The present invention relates to compositions and methods for the treatment and/or prevention of SARS. The invention further includes a method for the treatment and/or prevention of SARS through the administration of a therapeutically effective amount of at least one antiviral compound from among those described in the US patents and published international patent applications listed in Table 1 and Table 2. In one embodiment of the method, the antiviral compound is a small molecule. In another embodiment, the antiviral compound is a protease inhibitor. In a further embodiment, the antiviral protease inhibitor is a 3C-like protease inhibitor and/or a papain-like protease inhibitor. Combined treatment with the lopinavir/ritonavir (Kaletra) protease inhibitor and ribavirin has shown a favorable clinical response (Chu et al. (2004) Thorax 59:252-256). In another embodiment, the antiviral compound is an inhibitor of an RNA dependent RNA polymerase. In another embodiment, a first antiviral compound that is a protease inhibitor is administered with a second antiviral compound that is an RNA-dependent RNA polymerase inhibitor. The invention further provides for the administration of a steroidal anti-inflammatory drug in combination with at least one antiviral compound, for example, from the antiviral compounds described in the documents listed in Table 1 and Table 2. A combination treatment of steroids and ribavirin has been described by Fujii et al. (2004) J Infect Chemother 10:1-7. A combination treatment of corticosteroids and interferon alfacon-1 has also been reported (Loutfy et al. (2003) JAMA 290:3222-3228).

The invention further provides for a method for the treatment and/or prevention of SARS through the administration of a therapeutically effective amount of at least one antiviral compound from among those described in the US Patents and published international patent applications listed in Table 1 and Table 2 by inhalation. In another aspect, the antiviral compound may be administered in combination with a SMIP, SMIS, or other immunomodulatory compound such as those in Table 34 and in Table 35. In one embodiment of the method, the antiviral compound is a small molecule. In another embodiment, the antiviral compound is a protease inhibitor. In a further embodiment, the antiviral protease inhibitor is a 3C-like protease inhibitor and/or a papain-like protease inhibitor. In another embodiment, the antiviral compound is an inhibitor of an RNA dependent RNA polymerase. In another embodiment, a first antiviral compound that is a protease inhibitor is administered with a second antiviral compound that is an RNA-dependent RNA polymerase inhibitor. The invention further provides for the administration of a steroidal anti-inflammatory drug in combination with at least one antiviral compound, for example, from the antiviral compounds described in the documents listed in Table 1 and Table 2. The steroidal anti-inflammatory drug may be administered by inhalation for a local effect or administered for systemic absorption such as via an oral or intravenous route.

The invention further provides for methods for treating SARS infection comprising administering a small molecule immunopotentiator (SMIP) compound either alone or in combination with an antiviral compound or in combination with a SARS vaccine. In a further embodiment, the SMIP is a compound disclosed herein or set forth in Table 34.

The invention further provides for methods for treating SARS infection comprising administering an immunosuppressant compound, optionally a small molecule suppressant (SMIS) compound either alone or in combination with an antiviral compound. In a further embodiment, the immunosuppressant compound is disclosed herein or set forth in Table 35.

The invention further provides peptidic immunomodulating compositions, that include oligo and polypeptides, capable of effecting inflammatory response in a patient. In one embodiment, the peptidic immunomodulating composition is able to stimulate human cells to produce cytokines. In another embodiment the peptidic immunomodulating composition is capable of decreasing cytokine levels in the human. Preferred Examples of peptidic immunomodulating compositions include those listed in Table 35, as well as TGFβ2, TGFβ1, TGFβ3, thymopentin (TP5), β-mercaptopropionyl-arginyl-lysyl-aspartyl-valyl-tyrosyl-cysteine amide, colostrinine, lactoferrin (LF), cyclolinopeptide A (CLA), and tuftsin (TKPR). The peptidic immunomodulating compositions of the invention may be used alone or in combination with other agents, preferably antiviral compounds, for the treatment of SARS.

The invention further provides for a kit for use by a consumer for the treatment and/or prevention of SARS. Such a kit comprises: a) a pharmaceutical composition comprising a therapeutically effective amount of at least one antiviral, SMIP, SMIS, or other immunomodulating compound from among those described in the US patents and published international patent applications listed in Table 1, Table 2, Table 34 and Table 35 and a pharmaceutically acceptable carrier, vehicle or diluent; b) a container for holding the pharmaceutical composition; and, optionally, c) instructions describing a method of using the pharmaceutical compositions for the treatment and or the prevention of SARS. The kit may optionally contain a plurality of compounds for the treatment of SARS wherein the antiviral compounds are selected from 3C-like protease inhibitors and papain-like protease inhibitors. In a further embodiment, the kit contains an antiviral compound that is an RNA-dependent RNA polymerase inhibitor. When the kit comprises more than one antiviral, SMIP, SMIS, or other immunomodulating compound, the compounds contained in the kit may be optionally combined in the same pharmaceutical composition.

An additional aspect of the invention provides for the use of at least-one of the antiviral, SMIP, SMIS, or other immunomodulating compounds described in the US Patents and published international patent applications listed in Table 1, Table 2, Table 34 and Table 35 for the manufacture of a medicament for the treatment or prevention of SARS.

An additional aspect of the invention provides for the use of at least one SMIP compound, or at least one immunosuppressant compound, or at least one SMIS compound for the manufacture of a medicament for the treatment or prevention of SARS. Preferred SMIP, immunosuppressant, and SMIS compounds are described herein.

Unless otherwise specified, the following terms, when used within Section VI: “Compositions and Methods for Treatment of SARS Virus Infection” of the present application have the meanings as defined below:

As used herein, “limit”, “treat” and “treatment” are interchangeable terms as are “limiting” and “treating” and, as used herein, include preventative (e.g., prophylactic) and palliative treatment or the act of providing preventative or palliative treatment. The terms include a postponement of development of SARS symptoms and/or a reduction in the severity of such symptoms that will or are expected to develop following infection with a SARS virus. The terms further include ameliorating existing SARS symptoms, preventing additional symptoms, ameliorating or preventing the underlying metabolic causes of symptoms.

Representative uses of the compositions and methods of the present invention include: the elimination or reduction of the viral load of the SARS virus in a vertebrate, including humans, the elimination or reduction of symptoms associated with SARS, and a reduction in morbidity associated with SARS. In a SARS patient population, the use of the compositions and methods of the invention will result in the reduction in the high mortality rates associated with SARS.

Infection with the SARS virus and the symptoms associated with SARS can be treated in a subject by administering the compositions of the invention. The compositions of the invention may be administered systemically. For systemic use, the compounds herein are formulated for parenteral (e.g., intravenous, subcutaneous, intramuscular, intraperitoneal, intranasal or transdermal) or enteral (e.g., oral or rectal) delivery according to conventional methods. Intravenous administration can be by a series of injections or by continuous infusion over an extended period. Administration by injection or other routes of discretely spaced administration can be performed at intervals ranging from weekly to once to three times daily or more. Alternatively, the compositions disclosed herein may be administered in a cyclical manner (administration of disclosed composition, followed by no administration, followed by administration of disclosed compositions, and the like). Treatment will continue until the desired outcome is achieved.

A “subject” is a vertebrate animal including a human that is in need of treatment with the compositions, methods and kits of the present invention. The term “subject” or “subjects” is intended to refer to both the male and female gender unless one gender is specifically indicated.

“Coadministration” of a combination of a plurality of antiviral compounds means that these components can be administered together as a composition or as part of the same, unitary dosage form. “Co-administration” also includes administering a plurality of antiviral compounds separately but as part of the same therapeutic treatment program or regimen. “Co-administration” also includes administering a plurality of other agents, such as, for example an oligopeptide, a polypeptide, a peptidic immunomodulator, nucleic acid, antibodies, or a vaccine wherein the compounds or agents are administered separately but as part of the same therapeutic treatment program or regimen. The components need not necessarily be administered at essentially the same time, although they can if so desired. “Co-administration” also includes separate administration at different times and in any order. For example, where appropriate a patient may take one or more component(s) of the treatment in the morning and the one or more of the other component(s) at night.

By “antiviral compound” as used herein is meant an antiviral compound as described in the US patents and published international patent applications listed in Table 1 and Table 2. The US patents and published international patent applications listed in Table 1, Table 2 and Table 35 are incorporated herein in their entirety. In one embodiment, the antiviral compound is an RNA-dependent RNA polymerase. In another preferred embodiment the antiviral compound is a 3C-like protease inhibitor or a papain-like protease inhibitor. The antiviral compounds may be administered in the form of the acid, or of a soluble alkali metal salt or alkaline earth metal salt where appropriate.

The precise dosage of the antiviral compound will vary with the dosing schedule, the oral potency of the particular antiviral compound chosen, the age, size, sex and condition of the subject, the severity of the disorder to be treated, and other relevant medical and physical factors. Thus, a precise pharmaceutically effective amount cannot be specified in advance and can be readily determined by the caregiver or clinician.

Generally, an appropriate amount of antiviral compound is chosen to obtain a reduction in the load of the SARS virus in the subject and/or to obtain a reduction in the symptoms associated with SARS. For humans, an effective oral dose of antiviral compound is typically from about 1.5 to about 6000 μg/kg body weight per day and preferably about 10 to about 2000 μg/kg of body weight per day.

One of ordinary skill in the art will recognize that certain antiviral, SMIP, SMIS, and immunomodulating compounds of the invention including 3C-like protease inhibitors, papain-like protease inhibitators, and RNA-dependent RNA polymerase inhibitors will contain one or more atoms that may be in a particular stereochemical, tautomeric, or geometric configuration, giving rise to stereoisomers, tautomers and configurational isomers. All such isomers and mixtures thereof are included in this invention, when active. Crystalline and amorphous forms of the antiviral compounds of this invention are also included as are hydrates, solvates, polymorphs, and isomorphs of the antiviral compounds of the invention.

SMIP compounds of the invention include compounds are described in issued U.S. Pat. Nos. 4,547,511 and 4,738,971 with the general structure (a): 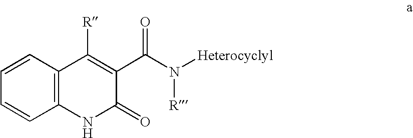
for the treatment of disorders responsive to agents that enhance cell-mediated immunity.

Immunostimulatory oligonucleotides and polynucleotides are described in PCT WO 98/55495 and PCT WO 98/16247. U.S. Patent Application No. 2002/0164341 describes adjuvants including an unmethylated CpG dinucleotide (CpG ODN) and a non-nucleic acid adjuvant. U.S. Patent Application No. 2002/0197269 describes compositions comprising an antigen, an antigenic CpG-ODN and a polycationic polymer.

Additionally, issued U.S. Pat. Nos. 4,689,338, 5,389,640, 5,268,376, 4,929,624, 5,266,575, 5,352,784, 5,494,916, 5,482,936, 5,346,905, 5,395,937, 5,238,944, 5,525,612, WO99/29693 and U.S. Ser. No. 09/361,544 disclose compounds of the general structure (b): 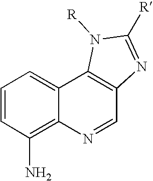
for the use as “immune response modifiers.”

Further compounds with SMIP and antiviral activity are described below and in US patent application entitled Thiosemicarbazones as Anti-Virals and Immunopotentiators filed on Dec. 29, 2003 with an attorney docket number of PP19814.004US generally disclosing compounds of the following structures:

A compound of formula c:

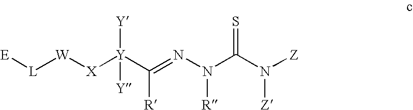

wherein: E is absent or selected from the group consisting of alkyl, substituted alkyl, cycloalkyl, substituted cycloalkyl, aryl, substituted aryl, heterocyclyl, substituted heterocyclyl, heteroaryl, and substituted heteroaryl;

L is absent or is selected from the group consisting of oxo, amino, alkylene, substituted alkylene, alkoxy, alkylamino, aminoalkyl, heterocyclyl, carbocyclyl, and carbonyl;

W is absent or selected from the group consisting of cycloalkyl, substituted cycloalkyl, aryl, substituted aryl, heterocyclyl, substituted heterocyclyl, heteroaryl, and substituted heteroaryl;

X is absent or is selected from the group consisting of oxo, amino, alkylene, substituted alkylene, alkoxy, alkylamino, aminoalkyl, heterocyclyl, carbocyclyl, and carbonyl;

Y is selected from the group consisting of cycloalkyl, substituted cycloalkyl, aryl, substituted aryl, heterocyclyl, substituted heterocyclyl, heteroaryl, and substituted heteroaryl;

Y′ is absent or is selected from the group consisting of F, Cl, Br, I, nitro, alkyl, substituted alkyl, and optionally substituted heterocyclyl, amino, alkylamino, dialkylamino;

Y″ is absent or is selected from the group consisting of F, Cl, Br, I, nitro, alkyl, substituted alkyl, and optionally substituted heterocyclyl, amino, alkylamino, dialkylamino;

R′ is H, alkyl, or substituted alkyl;

R″ is H, or

R′ and R″ are taken together to form a hetercyclic ring;

Z and Z′ are independently selected from the group consisting of hydrogen, alkyl, substituted alkyl, aryl, substituted aryl, arylalkyl, substituted arylalkyl, heteroaryl, substituted heteroaryl, heteroarylalkyl, substituted heteroarylalkyl, alkoxy, substituted alkoxy, aminocarbonyl, alkoxycarbonyl, carboxyl sulfonyl, methanesulfonyl, and substituted or unsubstituted alkylcarbonyl, arylcarbonyl, aralkylcarbonyl, heteroarylcarbonyl, heteroaralkylcarbonyl, alkylcarbonyloxy, arylcarbonyloxy, aralkylcarbonyloxy, heteroarylcarbonyloxy, heteroaralkylcarbonyloxy, alkylaminocarbonyloxy, arylaminocarbonyloxy, formyl, loweralkylcarbonyl, loweralkoxycarbonyl, aminocarbonyl, aminoaryl, alkylsulfonyl, sulfonamido, aminoalkoxy, alkylamino, heteroarylamino, alkylcarbonylamino, alkylaminocarbonylamino, arylaminocarbonylamino, aralkylcarbonylamino, heteroarylcarbonylamino, arylcarbonylamino, cycloamidino, cycloalkyl, cycloimido, arylsulfonyl and arylsulfonamido; or

Z and Z′ are taken together to form a heterocyclic group, that may be optionally substituted and the tautomers and the pharmaceutically acceptable salts, esters, or prodrugs thereof.

Further SMIP compounds are described below and in U.S. patent application Ser. No. 10/762,873, Use of Tryptanthrin Compounds for Immune Potentiation, filed on Jan. 21, 2004 and disclosing the general embodiment of compounds represented by Formula (d): 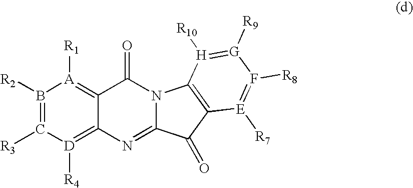
wherein

A, B, C, D, E, F, G, and H are independently selected from carbon and nitrogen, or A and B and/or C and D can be taken together to be nitrogen or sulfur;

R₁, R₂, R₃, R₄, R₈, and R₁₀ are independently selected from the group consisting of hydrogen, halogen, loweralkyl, alkyl, substituted alkyl, cycloalkyl, heterocyclyl, alkylheterocyclyl, substituted heterocyclyl, substituted alkenyl, amino, (substituted alkyl)(alkyl)amino, imino, haloloweralkyl, hydroxy, alkoxy, substituted alkoxy, hydroxyalkylthio, nitro, alkylsulfonyl, N-alkylsulfonamide, arylalkyl, arylalkylaryl, arylaryl, aryloxy, arylamino, acylamino, acyloxyamino, alkylaminoacylamino, alkylaminosulfonylamino, alkylamino, alkenylamino, dialkylamino, alkoxyalkylamino, alkoxyalkylheterocyclyl, mercaptoalkoxyalkyl, cyano, formyl, —COOR₁₁ wherein R₁₁ is hydrogen, loweralkyl, aryl, heterocyclyl, monosaccharide or disaccharide, and —CONR₁₂R₁₃ wherein R₁₂ and R₁₃ are independently selected from hydrogen, loweralkyl, aryl, heterocyclyl, saccharide, peptide and amino acid residues; or R₂ and R₃ taken together form a six membered aromatic ring;

R₇ and R₉ are independently selected from hydrogen, halogen, loweralkyl, haloloweralkyl, cycloalkyl, heterocyclyl, substituted heterocyclyl or heterocyclylalkyl; and

R₁, R₂, R₃, R₄, R₇, R₈, R₉, and R₁₀ are absent when the ring atom to which they would otherwise be bonded is sulfur or double-bonded nitrogen; or

the a pharmaceutically acceptable salts, esters, or prodrugs thereof, provided that R₁, R₂, R₃, R₄, R₇, R₈, R₉, and R₁₀ are not all hydrogen when A, B, C, D, E, F, and H are carbon.

In one embodiment, the compounds of Formula (I) have a backbone structure wherein D is nitrogen, and A-C and E-H are carbon.

In one embodiment, when D is carbon, at least one, or at least two of R₁—R₄, and R₇—R₁₀ are not hydrogen.

In one embodiment, R₁ through R₄, and R₈ and R₁₀ are independently selected from at least two of the group consisting of hydrogen, halogen, loweralkyl, cycloalkyl, heterocyclyl, substituted heterocyclyl, alkylheterocyclyl, amino, imino, haloloweralkyl, alkoxy, nitro, alkylsulfonyl, arylalkyl, arylalkylaryl, arylaryl, aryloxy, arylamino, acylamino, acyloxyamino, alkylaminoacylamino, alkylaminosulfonylamino, alkylamino, alkenylamino, dialkylamino, alkoxyalkylamino, alkoxyalkylheterocyclyl, mercaptoalkoxyalkyl, cyano, formyl, —COOR₁₁ where R₁₁ is hydrogen, loweralkyl, aryl, heterocyclyl, monosaccharide or disaccharide, and —CONR₁₂R₁₃ where R₁₂ and R₁₃ are independently selected from hydrogen, loweralkyl, aryl, heterocyclyl, saccharide, peptide and amino acid residues; and R₄ is not present when D is nitrogen.

In an additional embodiment, 4A, B, C, D, E, F, G, and H are independently selected from carbon and nitrogen;

R₁, R₂, R₃, R₄, R₈ and R₁₀ are independently selected from the group consisting of hydrogen, halogen, loweralkyl, alkyl, substituted alkyl, heterocyclyl, substituted heterocyclyl, substituted alkenyl, (substituted alkyl)(alkyl)amino, haloloweralkyl, hydroxy, alkoxy, substituted alkoxy, hydroxyalkylthio, nitro, N-alkylsulfonamide, cyano, —COOR₁₁ wherein R₁₁ is hydrogen, loweralkyl, aryl, heterocyclyl, monosaccharide or disaccharide, and —CONR₁₂R₁₃ wherein R₁₂ and R₁₃ are independently selected from hydrogen, loweralkyl, aryl, heterocyclyl, saccharide, peptide and amino acid residues.

For the compounds described herein:

The term “loweralkyl” refers to branched or straight chain acyclical alkyl groups comprising one to ten carbon atoms, including, e.g., methyl, ethyl, propyl, isopropyl, n-butyl, t-butyl, neopentyl and the like.

The term “alkyl” refers to alkyl groups that do not contain heteroatoms. Thus the term includes straight chain alkyl groups such as methyl, ethyl, propyl, butyl, pentyl, hexyl, heptyl, octyl, nonyl, decyl, undecyl, dodecyl and the like. The phrase also includes branched chain isomers of straight chain alkyl groups, including but not limited to, the following that are provided by way of example: —CH(CH₃)₂, —CH(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₃)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃), —CH(CH₃)CH₂CH(CH₃)₂, —CH(CH₃)CH(CH₃)CH(CH₃)₂, —CH(CH₂CH₃)CH(CH₃)CH(CH₃)(CH₂CH₃), and others. The term also includes cyclic alkyl groups such as cyclopropyl, cyclobutyl, cyclopentyl, cyclohexyl, cycloheptyl, and cyclooctyl and such rings substituted with straight and branched chain alkyl groups as defined above. The term also includes polycyclic alkyl groups such as, but not limited to, adamantyl norbornyl, and bicyclo[2.2.2]octyl and such rings substituted with straight and branched chain alkyl groups as defined above. Thus, the phrase unsubstituted alkyl groups includes primary alkyl groups, secondary alkyl groups, and tertiary alkyl groups. Unsubstituted alkyl groups may be bonded to one or more carbon atom(s), oxygen atom(s), nitrogen atom(s), and/or sulfur atom(s) in the parent compound. Preferred unsubstituted alkyl groups include straight and branched chain alkyl groups and cyclic alkyl groups having 1 to 20 carbon atoms. More preferred such unsubstituted alkyl groups have from 1 to 10 carbon atoms while even more preferred such groups have from 1 to 5 carbon atoms. Most preferred unsubstituted alkyl groups include straight and branched chain alkyl groups having from 1 to 3 carbon atoms and include methyl, ethyl, propyl, and —CH(CH₃)₂.

The phrase “substituted alkyl” refers to an unsubstituted alkyl group as defined above in which one or more bonds to a carbon(s) or hydrogen(s) are replaced by a bond to non-hydrogen and non-carbon atoms such as, but not limited to, a halogen atom in halides such as F, Cl, Br, and I; a phosphorus atom in groups such as phosphate and dialkyl alkylphosphonate; oxygen atom in groups such as hydroxyl groups, alkoxy groups, aryloxy groups, and ester groups; a sulfur atom in groups such as thiol groups, alkyl and aryl sulfide groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as in trialkylsilyl groups, dialkylarylsilyl groups, alkyldiarylsilyl groups, and triarylsilyl groups; and other heteroatoms in various other groups. Substituted alkyl groups also include groups in which one or more bonds to a carbon(s) or hydrogen(s) atom is replaced by a bond to a heteroatom such as oxygen in carbonyl, carboxyl, and ester groups; nitrogen in groups such as imines, oximes, hydrazones, and nitriles. Preferred substituted alkyl groups include, among others, alkyl groups in which one or more bonds to a carbon or hydrogen atom is/are replaced by one or more bonds to fluorine atoms. One example of a substituted alkyl group is the trifluoromethyl group and other alkyl groups that contain the trifluoromethyl group. Other alkyl groups include those in which one or more bonds to a carbon or hydrogen atom is replaced by a bond to an oxygen atom such that the substituted alkyl group contains a hydroxyl, alkoxy, aryloxy group, or heterocyclyloxy group. Still other alkyl groups include alkyl groups that have an amine, alkylamine, dialkylamine, arylamine, (alkyl)(aryl)amine,diarylamine, heterocyclylamine, (alkyl)(heterocyclyl)amine, (aryl)(heterocyclyl)amine, or diheterocyclylamine group.

The term “alkoxy” refers to RO— wherein R, for example, is alkyl such as loweralkyl defined above. Representative examples of loweralkyl alkoxy groups include methoxy, ethoxy, t-butoxy and the like.

The phrase “substituted alkoxy” refers to RO—, where R is, for example, an alkyl substituted, for example, with a halogen. RO is for example OCF₃.

The term “alkenyl” refers to a branched or straight chain groups comprising two to twenty carbon atoms that also comprises one or more carbon-carbon double bonds. Representative alkenyl groups include prenyl, 2-propenyl (i.e., allyl), 3-methyl-2-butenyl, 3,7-dimethyl-2,6-octadienyl, 4,8-dimethyl-3,7-nonadienyl, 3,7,11-trimethyl-2,6,10-dodecatrienyl and the like.

The phrase “substituted alkenyl” refers to alkenyl groups that are substituted, for example, diethyl hex-5-enylphosponate, and others with an alkyl or substituted alkyl group such as dialkyl phosphate or an ester such as an acetate ester.

The phrase “dialkyl amino” refers to an amino group substituted with two alkyl groups such as C1-20 alkyl groups.

The phrase “substituted dialkyl amino” refers to a dialkylamino substituted, for example, with a carboxylic acid, ester, hydroxy or alkoxy.

The term “hydroxyalkylthio” refers to a thio radical to which is appended a hydroxyalkyl group, where the alkyl is for example lower alkyl. An example is hydroxyethylthio, —SCH₂CH₂OH.

The term “N-alkylsulfonamide” refers to the group —SO₂NHalkyl, where alkyl is, for example, octyl.

The term “alkynyl” refers to a branched or straight chain comprising two to twenty carbon atoms that also comprises one or more carbon-carbon triple bonds. Representative alkynyl groups include ethynyl, 2-propynyl (propargyl), 1-propynyl and the like.

The term “aryl” refers to aryl groups that do not contain heteroatoms. Thus the term includes, but is not limited to, groups such as phenyl, biphenyl, anthracenyl, naphthenyl by way of example. Although the phrase “unsubstituted aryl” includes groups containing condensed rings such as naphthalene, it does not include aryl groups that have other groups such as alkyl or halo groups bonded to one of the ring members, as aryl groups such as tolyl are considered herein to be substituted aryl groups as described below. A preferred unsubstituted aryl group is phenyl. Unsubstituted aryl groups may be bonded to one or more carbon atom(s), oxygen atom(s), nitrogen atom(s), and/or sulfur atom(s) in the parent compound, however.

The phrase “substituted aryl group” has the same meaning with respect to aryl groups that substituted alkyl groups had with respect to alkyl groups. However, a substituted aryl group also includes aryl groups in which one of the aromatic carbons is bonded to one of the non-carbon or non-hydrogen atoms described above and also includes aryl groups in which one or more aromatic carbons of the aryl group is bonded to a substituted and/or unsubstituted alkyl, alkenyl, or alkynyl group as defined herein. This includes bonding arrangements in which two carbon atoms of an aryl group are bonded to two atoms of an alkyl, alkenyl, or alkynyl group to define a fused ring system (e.g. dihydronaphthyl or tetrahydronaphthyl). Thus, the phrase “substituted aryl” includes, but is not limited to tolyl, and hydroxyphenyl among others.

The term “arylalkyl” refers to a loweralkyl radical to which is appended an aryl group. Representative arylalkyl groups include benzyl, phenylethyl, hydroxybenzyl, fluorobenzyl, fluorophenylethyl and the like.

The phrase “unfused arylaryl” refers to a group or substituent to which two aryl groups, that are not condensed to each other, are bound. Exemplary unfused arylaryl compounds include, for example, phenylbenzene, diphenyldiazene, 4-methylthio-1-phenylbenzene, phenoxybenzene, (2-phenylethynyl)benzene, diphenyl ketone, (4-phenylbuta-1,3-diynyl)benzene, phenylbenzylamine, (phenylmethoxy)benzene, and the like. Preferred substituted unfused arylaryl groups include: 2-(phenylamino)-N-[4-(2-phenylethynyl)phenyl]acetamide, 1,4-diphenylbenzene, N-[4-(2-phenylethynyl)phenyl]-2-[benzylamino]acetamide, 2-amino-N-[4-(2-phenylethynyl)phenyl]propanamide, 2-amino-N-[4-(2-phenylethynyl)phenyl]acetamide, 2-(cyclopropylamino)-N-[4-(2-phenylethynyl)phenyl]acetamide, 2-(ethylamino)-N-[4-(2-phenylethynyl)phenyl]acetamide, 2-[(2-methylpropyl)amino]-N-[4-(2-phenylethynyl)phenyl]acetamide, 5-phenyl-2H-benzo[d]1,3-dioxolene, 2-chloro-1-methoxy-4-phenylbenzene, 2-[(imidazolylmethyl)amino]-N-[4-(2-phenylethynyl)phenyl]acetamide, 4-phenyl-1-phenoxybenzene, N-(2-aminoethyl)[4-(2-phenylethynyl)phenyl]carboxamide, 2-{[(4-fluorophenyl)methyl]amino}-N-[4-(2-phenylethynyl)phenyl]acetamide, 2-{[(4-methylphenyl)methyl]amino}-N-[4-(2-phenylethynyl)phenyl]acetamide, 4-phenyl-1-(trifluoromethyl)benzene, 1-butyl-4-phenylbenzene, 2-(cyclohexylamino)-N-[4-(2-phenylethynyl)phenyl]acetamide, 2-(ethylmethylamino)-N-[4-(2-phenylethynyl)phenyl]acetamide, 2-(butylamino)-N-[4-(2-phenylethynyl)phenyl]acetamide, N-[4-(2-phenylethynyl)phenyl]-2-(4-pyridylamino)acetamide, N-[4-(2-phenylethynyl)phenyl]-2-(quinuclidin-3-ylamino)acetamide, N-[4-(2-phenylethynyl)phenyl]pyrrolidin-2-ylcarboxamide, 2-amino-3-methyl-N-[4-(2-phenylethynyl)phenyl]butanamide, 4-(4-phenylbuta-1,3-diynyl)phenylamine, 2-(dimethylamino)-N-[4-(4-phenylbuta-1,3-diynyl)phenyl]acetamide, 2-(ethylamino)-N-[4-(4-phenylbuta-1,3-diynyl)phenyl]acetamide, 4-ethyl-1-phenylbenzene, 1-[4-(2-phenylethynyl)phenyl]ethan-1-one, N-(1-carbamoyl-2-hydroxypropyl)[4-(4-phenylbuta-1,3-diynyl)phenyl]carboxamide, N-[4-(2-phenylethynyl)phenyl]propanamide, 4-methoxyphenyl phenyl ketone, phenyl-N-benzamide, (tert-butoxy)-N-[(4-phenylphenyl)methyl]carboxamide, 2-(3-phenylphenoxy)ethanehydroxamic acid, 3-phenylphenyl propanoate, 1-(4-ethoxyphenyl)-4-methoxybenzene, and [4-(2-phenylethynyl)phenyl]pyrrole.

The phrase “unfused heteroarylaryl” refers to a unfused arylaryl group where one of the aryl groups is a heteroaryl group. Exemplary heteroarylaryl groups include, for example, 2-phenylpyridine, phenylpyrrole, 3-(2-phenylethynyl)pyridine, phenylpyrazole, 5-(2-phenylethynyl)-1,3-dihydropyrimidine-2,4-dione, 4-phenyl-1,2,3-thiadiazole, 2-(2-phenylethynyl)pyrazine, 2-phenylthiophene, phenylimidazole, 3-(2-piperazinylphenyl)furan, 3-(2,4-dichlorophenyl)-4-methylpyrrole, and the like. Preferred substituted unfused heteroarylaryl groups include: 5-(2-phenylethynyl)pyrimidine-2-ylamine, 1-methoxy-4-(2-thienyl)benzene, 1-methoxy-3-(2-thienyl)benzene, 5-methyl-2-phenylpyridine, 5-methyl-3-phenylisoxazole, 2-[3-(trifluoromethyl)phenyl]furan, 3-fluoro-5-(2-furyl)-2-methoxy-1-prop-2-enylbenzene, (hydroxyimino)(5-phenyl(2-thienyl))methane, 5-[(4-methylpiperazinyl)methyl]-2-phenylthiophene, 2-(4-ethylphenyl)thiophene, 4-methylthio-1-(2-thienyl)benzene, 2-(3-nitrophenyl)thiophene, (tert-butoxy)-N-[(5-phenyl(3-pyridyl))methyl]carboxamide, hydroxy-N-[(5-phenyl(3-pyridyl))methyl]amide, 2-(phenylmethylthio)pyridine, and benzylimidazole.

The phrase “unfused heteroarylheteroaryl” refers to an unfused arylaryl group where both of the aryl groups is a heteroaryl group. Exemplary heteroarylheteroaryl groups include, for example, 3-pyridylimidazole, 2-imidazolylpyrazine, and the like. Preferred substituted unfused heteroarylheteroaryl groups include: 2-(4-piperazinyl-3-pyridyl)furan, diethyl(3-pyrazin-2-yl(4-pyridyl))amine, and dimethyl {2-[2-(5-methylpyrazin-2-yl)ethynyl](4-pyridyl)}amine.

The phrase “fused arylaryl” refers to an aryl group as previously defined that is condensed, and fully conjugated to an aryl group. Representative fused arylaryl groups include biphenyl, 4-(1-naphthyl)phenyl, 4-(2-naphthyl)phenyl and the like.

The phrase “fused heteroarylaryl” refers to an aryl group as previously defined that is condensed, and fully conjugated with a heteroaryl group. Representative fused heteroarylaryl groups include quinoline, quinazoline and the like.

The phrase “fused heteroarylheteroaryl” refers to a heteroaryl group as previously defined that is condensed, and fully conjugated with another heteroaryl group. Representative fused heteroarylheteroaryl groups include pyrazalopyrimidine, imidazoquinoline and the like.

The term “aryloxy” refers to RO— wherein R is an aryl group. Representative arylalkoxy group include benzyloxy, phenylethoxy and the like.

The term “arylalkoxy” refers to a lower alkoxy radical to which is appended an aryl group. Representative arylalkoxy group include benzyloxy, phenylethoxy and the like.

The term “aryloxyaryl” refers to an aryl radical to which is appended an aryloxy group. Representative aryloxyaryl groups include 4-phenoxyphenyl, 3-phenoxyphenyl, 4-phenoxy-1-naphthyl, 3-phenoxy-1-naphthyl and the like.

The term “aryloxyarylalkyl” refers to an arylalkyl radical to which is appended an aryloxy group. Representative aryloxyarylalkyl groups include 4-phenoxyphenylmethyl, 3-phenoxyphenylmethyl, 4-phenoxyphenylethyl, 3-phenoxy-phenylethyl and the like.

The term “arylalkoxyaryl” refers to an aryl radical to which is appended an arylalkoxy group. Representative arylalkoxyaryl groups include 4-benzyloxylphenyl, 3-benzyloxyphenyl and the like.

The term “arylalkoxyarylalkyl” refers to an arylalkyl radical to which is appended an arylalkoxy group. Representative arylalkoxyarylalkyl groups include 4-benzyloxylbenzyl, 3-benzyloxybenzyl and the like.

The term “cycloalkyl” refers to an alicyclic group comprising from 3 to 7 carbon atoms including, but not limited to, cyclopropyl, cyclobutyl, cyclopentyl, cyclohexyl and the like.

The term “cycloalkylalkyl” refers to a loweralkyl radical to which is appended a cycloalkyl group. Representative examples of cycloalkylalkyl include cyclopropylmethyl, cyclohexylmethyl, 2-(cyclopropyl)ethyl and the like.

The term “halogen” refers to iodine, bromine, chlorine or fluorine; “halo” refers to iodo, bromo, chloro or fluoro.

The term “haloalkyl” refers to a lower alkyl radical, as defined above, bearing at least one halogen substituent, for example, chloromethyl, fluoroethyl or trifluoromethyl and the like.

The term “heterocyclyl” (or heterocyclic, or heterocyclo) refers to both aromatic and nonaromatic ring compounds including monocyclic, bicyclic, and polycyclic ring compounds such as, but not limited to, quinuclidyl, containing 3 or more ring members of which one or more is a heteroatom such as, but not limited to, N, O, and S. Although the phrase “unsubstituted heterocyclyl” includes condensed heterocyclic rings such as benzimidazolyl, it does not include heterocyclyl groups that have other groups such as alkyl or halo groups bonded to one of the ring members as compounds such as 2-methylbenzimidazolyl are substituted heterocyclyl groups. Examples of heterocyclyl groups include, but are not limited to: unsaturated 3 to 8 membered rings containing 1 to 4 nitrogen atoms such as, but not limited to pyrrolyl, pyrrolinyl, imidazolyl, pyrazolyl, pyridyl, dihydropyridyl, pyrimidyl, pyrazinyl, pyridazinyl, triazolyl (e.g. 4H-1,2,4-triazolyl, 1H-1,2,3-triazolyl, 2H-1,2,3-triazolyl etc.), tetrazolyl, (e.g. 1H-tetrazolyl, 2H tetrazolyl, etc.); saturated 3 to 8 membered rings containing 1 to 4 nitrogen atoms such as, but not limited to, pyrrolidinyl, imidazolidinyl, piperidinyl, piperazinyl; condensed unsaturated heterocyclic groups containing 1 to 4 nitrogen atoms such as, but not limited to, indolyl, isoindolyl, indolinyl, indolizinyl, benzimidazolyl, quinolyl, isoquinolyl, indazolyl, benzotriazolyl; unsaturated 3 to 8 membered rings containing 1 to 2 oxygen atoms and 1 to 3 nitrogen atoms such as, but not limited to, oxazolyl, isoxazolyl, oxadiazolyl (e.g. 1,2,4-oxadiazolyl, 1,3,4-oxadiazolyl, 1,2,5-oxadiazolyl, etc.); saturated 3 to 8 membered rings containing 1 to 2 oxygen atoms and 1 to 3 nitrogen atoms such as, but not limited to, morpholinyl; unsaturated condensed heterocyclic groups containing 1 to 2 oxygen atoms and 1 to 3 nitrogen atoms, for example, benzoxazolyl, benzoxadiazolyl, benzoxazinyl (e.g. 2H-1,4-benzoxazinyl etc.); unsaturated 3 to 8 membered rings containing 1 to 3 sulfur atoms and 1 to 3 nitrogen atoms-such as, but not limited to, thiazolyl, isothiazolyl, thiadiazolyl (e.g. 1,2,3-thiadiazolyl, 1,2,4-thiadiazolyl, 1,3,4-thiadiazolyl, 1,2,5-thiadiazolyl, etc.); saturated 3 to 8 membered rings containing 1 to 2 sulfur atoms and 1 to 3 nitrogen atoms such as, but not limited to, thiazolodinyl; saturated and unsaturated 3 to 8 membered rings containing 1 to 2 sulfur atoms such as, but not limited to, thienyl, dihydrodithiinyl, dihydrodithionyl, tetrahydrothiophene, tetrahydrothiopyran; unsaturated condensed heterocyclic rings containing 1 to 2 sulfur atoms and 1 to 3 nitrogen atoms such as, but not limited to, benzothiazolyl, benzothiadiazolyl, benzothiazinyl (e.g. 2H-1,4-benzothiazinyl, etc.), dihydrobenzothiazinyl (e.g. 2H-3,4-dihydrobenzothiazinyl, etc.), unsaturated 3 to 8 membered rings containing oxygen atoms such as, but not limited to furyl; unsaturated condensed heterocyclic rings containing 1 to 2 oxygen atoms such as benzodioxolyl (e.g. 1,3-benzodioxoyl, etc.); unsaturated 3 to 8 membered rings containing an oxygen atom and 1 to 2 sulfur atoms such as, but not limited to, dihydrooxathiinyl; saturated 3 to 8 membered rings containing 1 to 2 oxygen atoms and 1 to 2 sulfur atoms such as 1,4-oxathiane; unsaturated condensed rings containing 1 to 2 sulfur atoms such as benzothienyl, benzodithiinyl; and unsaturated condensed heterocyclic rings containing an oxygen atom and 1 to 2 oxygen atoms such as benzoxathiinyl. Heterocyclyl group also include those described above in which one or more S atoms in the ring is double-bonded to one or two oxygen atoms (sulfoxides and sulfones). For example, heterocyclyl groups include tetrahydrothiophene, tetrahydrothiophene oxide, and tetrahydrothiophene 1,1-dioxide. Preferred heterocyclyl groups contain 5 or 6 ring members. More preferred heterocyclyl groups include morpholine, piperazine, piperidine, pyrrolidine, imidazole, pyrazole, 1,2,3-triazole, 1,2,4-triazole, tetrazole, thiomorpholine, thiomorpholine in which the S atom of the thiomorpholine is bonded to one or more O atoms, pyrrole, homopiperazine, oxazolidin-2-one, pyrrolidin-2-one, oxazole, quinuclidine, thiazole, isoxazole, furan, and tetrahydrofuran.

The phrase “substituted heterocyclyl” refers to an heterocyclyl group as defined above in which one of the ring members is bonded to a non-hydrogen atom such as described above with respect to substituted alkyl groups and substituted aryl groups. Examples, include, but are not limited to, 2-methylbenzimidazolyl, 5-methylbenzimidazolyl, 5-chlorobenzthiazolyl, 1-methyl piperazinyl, and 2-chloropyridyl among others.

“Aminosulfonyl” refers to the group -S(O) 2-NH2. “Substituted aminosulfonyl” refersto the group —S(O)₂—NRR′ where R is loweralkyl and R′ is hydrogen or a loweralkyl. The term “aralkylaminosulfonlyaryl” refers to the group -aryl-S(O)₂—NH-aralkyl, where the aralkyl is loweraralkyl.

“Carbonyl” refers to the divalent group —C(O)—.

“Carbonyloxy” refers generally to the group —C(O)—O—,. Such groups include esters, —C(O)—O—R, where R is loweralkyl, cycloalkyl, aryl, or loweraralkyl. The term “carbonyloxycycloalkyl” refers generally to both an “carbonyloxycarbocycloalkyl” and an “carbonyloxyheterocycloalkyl”, i.e., where R is a carbocycloalkyl or heterocycloalkyl, respectively. The term “arylcarbonyloxy” refers to the group —C(O)—O-aryl, where aryl is a mono- or polycyclic, carbocycloaryl or heterocycloaryl. The term “aralkylcarbonyloxy” refers to the group —C(O)—O-aralkyl, where the aralkyl is loweraralkyl.

The term “sulfonyl” refers to the group —SO₂—. “Alkylsulfonyl” refers to a substituted sulfonyl of the structure —SO₂R— in which R is alkyl. Alkylsulfonyl groups employed in compounds of the present invention are typically loweralkylsulfonyl groups having from 1 to 6 carbon atoms in its backbone structure. Thus, typical alkylsulfonyl groups employed in compounds of the present invention include, for example, methylsulfonyl (i.e., where R is methyl), ethylsulfonyl (i.e., where R is ethyl), propylsulfonyl (i.e., where R is propyl), and the like. The term “arylsulfonyl” refersto the group —SO₂-aryl. The term “aralkylsulfonyl” refers to the group —SO₂-aralkyl, in which the aralkyl is loweraralkyl. The term “sulfonamido” refers to —SO₂NH₂.

The term “carbonylamino” refers to the divalent group —NH—C(O)— in which the hydrogen atom of the amide nitrogen of the carbonylamino group can be replaced a loweralkyl, aryl, or loweraralkyl group. Such groups include moieties such as carbamate esters (—NH—C(O)—O—R) and amides —NH—C(O)—O—R, where R is a straight or branched chain loweralkyl, cycloalkyl, or aryl or loweraralkyl. The term “loweralkylcarbonylamino” refers to alkylcarbonylamino where R is a loweralkyl having from 1 to about 6 carbon atoms in its backbone structure. The term “arylcarbonylamino” refers to group —NH—C(O)—R where R is an aryl. Similarly, the term “aralkylcarbonylamino ” refers to carbonylamino where R is a lower aralkyl.

The term “guanidino” or “guanidyl” refers to moieties derived from guanidine, H2N—C(═NH)—NH₂. Such moieties include those bonded at the nitrogen atom carrying the formal double bond (the “2”-position of the guanidine, e.g., diaminomethyleneamino, (H2N)₂C═NH—) and those bonded at either of the nitrogen atoms carrying a formal single bond (the “1-” and/or “3”-positions of the guandine, e.g., H₂N—C(═NH)—NH—). The hydrogen atoms at any of the nitrogens can be replaced with a suitable substituent, such as loweralkyl, aryl, or loweraralkyl.

Representative cycloimido and heterocycloimido groups include, for example, those shown below. These cycloimido and heterocycloimido can be further substituted and may be attached at various positions as will be apparent to those having skill in the organic and medicinal chemistry arts in conjunction with the disclosure herein.

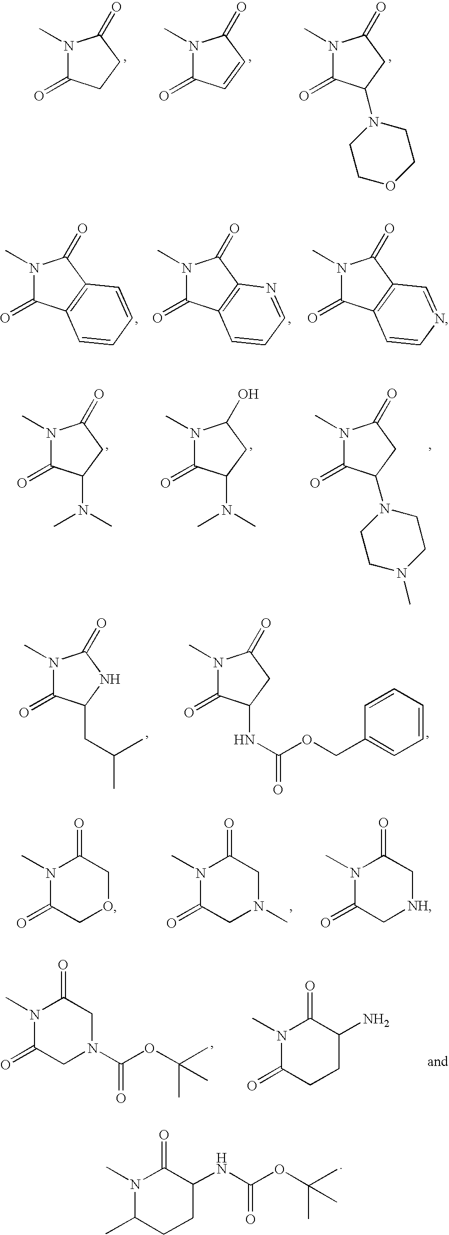

Representative substituted amidino and heterocycloamidino groups include, for example, those shown below. These amidino and heterocycloamidino groups can be further substituted as will be apparent to those having skill in the organic and medicinal chemistry arts in conjunction with the disclosure herein.

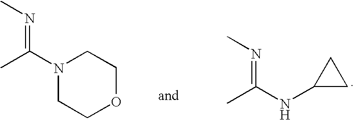

Representative substituted alkylcarbonylamino, alkyloxycarbonylamino, aminoalkyloxycarbonylamino, and arylcarbonylamino groups include, for example, those shown below. These groups can be further substituted as will be apparent to those having skill in the organic and medicinal chemistry arts in conjunction with the disclosure herein.

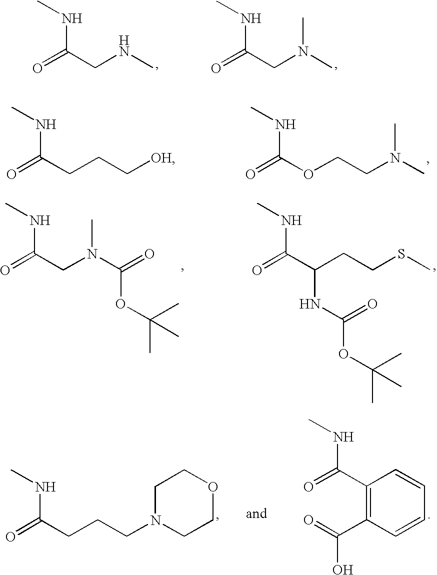

Representative substituted aminocarbonyl groups include, for example, those shown below. These can heterocyclo groups be further substituted as will be apparent to those having skill in the organic and medicinal chemistry arts in conjunction with the disclosure herein.

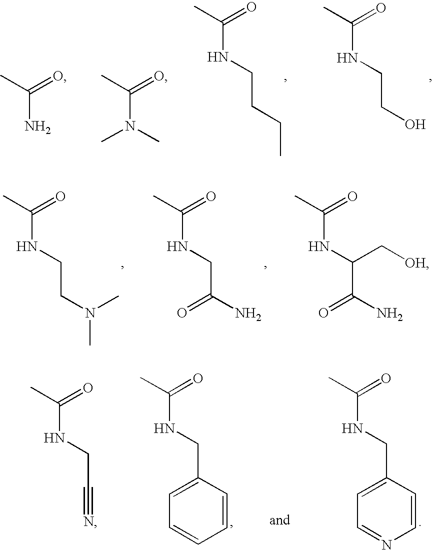

Representative substituted alkoxycarbonyl groups include, for example, those shown below. These alkoxycarbonyl groups can be further substituted as will be apparent to those having skill in the organic and medicinal chemistry arts in conjunction with the disclosure herein.

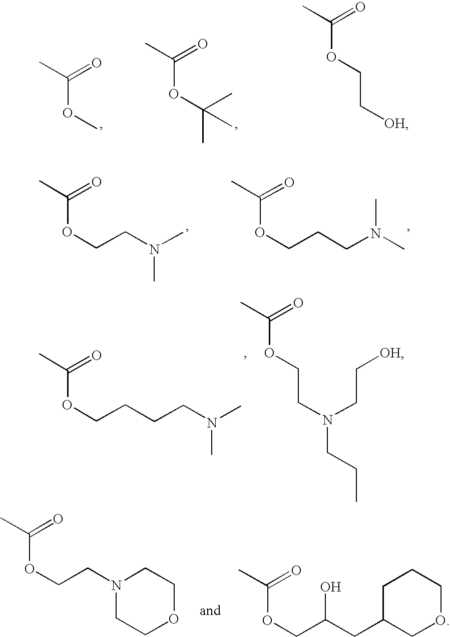

“Substituted” refers to the definite replacement of hydrogen with one or more monovalent or divalent radicals. Suitable substitution groups include, those described herein for particular groups, as well as hydroxyl, nitro, amino, imino, cyano, halo, thio, thioamido, amidino, imidino, oxo, oxamidino, methoxamidino, imidino, guanidino, sulfonamido, carboxyl, formyl, alkyl, substituted alkyl, haloloweralkyl, loweralkoxy, haloloweralkoxy, loweralkoxyalkyl, alkylcarbonyl, arylcarbonyl, aralkylcarbonyl, heteroarylcarbonyl, heteroaralkylcarbonyl, alkylthio, aminoalkyl, cyanoalkyl, benzyl, pyridyl, pyrazolyl, pyrrole, thiophene, imidazolyl, and the like.

The term “linking moiety” refers to a covalent bond or an uncyclized divalent group, such as, for example, —CO—, —O—, —S—, —CH₂—, —NH—, and substituted or unsubstituted alkyl, alkenyl, alkynyl, carbonyl, alkoxycarbonyl groups as defined herein.

The term “SMIP compound” refers to small molecule immunopotentiating compounds, that include small molecule compounds below about MW 1000 g/mol, preferably MW 800 g/mol that are capable of stimulating or modulating a pro-inflammatory response in a patient. In an embodiment, the SMIP compounds are able to stimulate human peripheral blood mononuclear cells to produce cytokines. Preferred SMIP compounds and derivatives thereof include, for example, aminoazavinyl compounds, benzazole compounds, acylpiperazine compounds, indoledione compounds, tetrahydroisoquinoline (THIQ) compounds, anthraquinone compounds, indanedione compounds, pthalimide compounds, benzocyclodione compounds, aminobenzimidazole quinolinone (ABIQ) compounds, hydraphthalimide compounds, pyrazolopyrimidine compounds, quinazilinone compounds, quinoxaline compounds, triazine compounds, tetrahydropyrrolidinoquinoxaline compounds, pyrrole compounds, benzophenone compounds, sterol compound, and isoxazole compounds.

The term “SMIS compound” refers to small molecule immunosuppressant compounds, that include small molecule compounds below about about MW 1000 g/mol, preferably MW 800 g/mol, capable of suppressing or modulating a pro-inflammatory response in a patient.

Acylpiperazine compounds as described throughout this application include compounds of formula (III) as shown below: 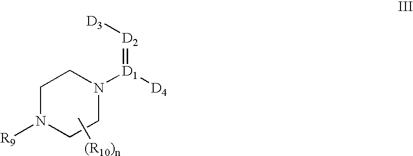
wherein,
R₉ is selected from the group consisting of substituted or unsubstituted aryl, heteroaryl, arylalkyl, arylalkenyl, heteroarylalkyl, and heteroarylalkenyl;
R₁₀ is substituted or unsubstituted alkyl;
n is an integer from 0-2; and
if D₁ is carbon than D₂ is oxygen, D₃ is absent, and D₄ is selected from the group consisting of substituted or unsubstituted aryl, heteroaryl, carbocycyl, alkoxyaryl, fused arylaryl, fused arylheteroaryl, and fused heteroarylaryl; or,
if D₁ is nitrogen than D₂ is nitrogen, D₄ is absent, and D₃ is selected from the group consisting of substituted or unsubstituted aryl, heteroaryl, carbocycyl, alkoxyaryl, fused arylaryl, fused arylheteroaryl, and fused heteroarylaryl.

Indoledione compounds as described throughout this application include compounds of formula (IV) as shown below: 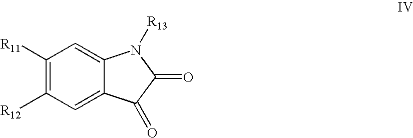
wherein,
R₁₁ and R₁₂ are independently selected from the group consisting of H, nitro, halogen, amino, hydroxy, cyano, carboxcyclic acid, and substituted or unsubstituted alkyl, aryl, heteroaryl, alkoxy, alkylcarbonyl, alkylcarbonylamino, alkylaminocarbonyl, aminocarbonyl, arylalkoxy, heteroarylalkoxy, alkylamino, arylalkylamino, arylamino, heteroarylamino, heteroarylaminoalkyl, heterocyclyl, heterocyclylalkoxy, heterocyclylalkyl, and carbocyclyl groups; and,
R₁₃ is selected from the group consisting of substituted or unsubstituted aryl, heteroaryl, arylalkyl, heteroarylalkyl, heterocyclyl, heterocyclylalkyl, and alkylbenzyl.

Tetrahydroisoquinoline (THIQ) compounds as described throughout this application include compounds of formula (V) as shown below: 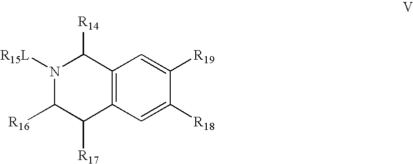
wherein,
L is a covalent bond or selected from the group consisting of —CH₂—, —CO—, —O—, —S—, CHF, —NH—, —NR₂₀—, where R₂₀ is lower alkyl;
R₁₄ is selected from the group consisting of hydrogen, halogen, and substituted or unsubstituted alkyl;
R₁₅ is selected from the group consisting of substituted or unsubstituted carbocyclyl, aryl, arylalkyl, alkoxyaryl, heteroaryl, heterocyclyl;
R₁₆ is selected from the group consisting of hydrogen, halogen, and substituted or unsubstituted alkyl;
R₁₇ is selected from the group consisting of hydrogen, halogen, and substituted or unsubstituted alkyl; and,
P₁₈ and R₁₉ are independently selected from the group consisting of H, hydroxy, halogen, alkoxy, amino, unsubstituted alkyl, substituted alkyl, and alkylamino.

Benzocyclodione compounds as described throughout this application include compounds of formula (VI) as shown below: 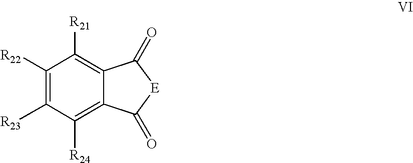
wherein,
E is selected from the group consisting of NR₂₅ or CR₂₆R₂₇;
R₂₁, R₂₃, and R₂₄ are independently selected from the group consisting of H, hydroxy, halogen, alkoxy, amino, unsubstituted alkyl, substituted alkyl, and alkylamino;
R₂₂ is selected from the group consisting or H, hydroxy, halogen, alkoxy, amino, and unsubstituted or substituted alkyl, and alkylamino, arylalkyl, heteroarylalkyl, aryl, heteroaryl, arylcarbonyl, heterocyclyl, heterocyclylalkyl, and heteroarylcarbonyl;
R₂₅ is selected from the group consisting of substituted or unsubstituted aryl, heteroaryl, heterocyclyl, carbocyclyl, arylalkyl, heteroarylalkyl, and heterocyclyalkyl;
R₂₆ is selected from the group consisting of H, halogen, hydroxy, amino, and substituted or unsubstituted alkyl, carbonylalkyl, and alkylcarbonylalkyl; and,
R₂₇ is selected from the group aryl, arylalkyl, heteroarylalkyl, heterocyclyl, heterocyclylalkyl, carbocyclyl, arylcarbonylalkyl, and arylalkylcarbonyl.

Aminoazavinyl compounds as described throughout this application include compounds of formula (VII) as shown below: 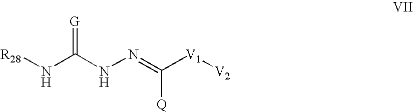
wherein,
G is either S or NH;
R₂₈ is selected from the group consisting of H, and substituted or unsubstituted alkyl, aryl, heteroaryl, heteroarylalkyl, arylalkyl, carbocyclyl, carbocyclylalkyl, heterocyclyl, and heterocyclylalkyl;
Q is selected from the group consisting of hydrogen, substituted alkyl, unsubstituted alkyl, and aryl, substituted aryl, heteroaryl, substituted heteroaryl, heterocyclyl, substituted heterocyclyl, fused or unfused arylaryl, substituted arylaryl, arylheteroaryl, substituted arylheteroaryl, heteroarylheteroaryl, and substituted heteroarylheteroaryl;
V₁ is selected from the group consisting of alkyl, substituted alkyl, aryl, substituted aryl, arylalkyl, substituted arylalkyl, heteroaryl, substituted heteroaryl, heteroarylalkyl, substituted heteroarylalkyl, alkoxy, substituted alkoxy, aminocarbonyl, alkoxycarbonyl, carboxyl sulfonyl, methanesulfonyl, and substituted or unsubstituted alkylcarbonyl, arylcarbonyl, aralkylcarbonyl, heteroarylcarbonyl, heteroaralkylcarbonyl, alkylcarbonyloxy, arylcarbonyloxy, aralkylcarbonyloxy, heteroarylcarbonyloxy, heteroaralkylcarbonyloxy, alkylaminocarbonyloxy, arylaminocarbonyloxy, formyl, loweralkylcarbonyl, loweralkoxycarbonyl, aminocarbonyl, aminoaryl, alkylsulfonyl, sulfonamido, aminoalkoxy, alkylamino, heteroarylamino, alkylcarbonylamino, alkylaminocarbonylamino, arylaminocarbonylamino, aralkylcarbonylamino, heteroarylcarbonylamino, arylcarbonylamino, cycloamidino, cycloalkyl, cycloimido, arylsulfonyl and arylsulfonamido; and,
V₂ is selected from the group consisting of hydrodgen, halogen, alkyl, substituted alkyl, aryl, substituted aryl, arylalkyl, substituted arylalkyl, heteroaryl, substituted heteroaryl, heteroarylalkyl, substituted heteroarylalkyl, alkoxy, substituted alkoxy, aminocarbonyl, alkoxycarbonyl, carboxyl sulfonyl, methanesulfonyl, and substituted or unsubstituted alkylcarbonyl, arylcarbonyl, aralkylcarbonyl, heteroarylcarbonyl, heteroaralkylcarbonyl, alkylcarbonyloxy, arylcarbonyloxy, aralkylcarbonyloxy, heteroarylcarbonyloxy, heteroaralkylcarbonyloxy, alkylaminocarbonyloxy, arylaminocarbonyloxy, formyl, loweralkylcarbonyl, loweralkoxycarbonyl, aminocarbonyl, aminoaryl, alkylsulfonyl, sulfonamido, aminoalkoxy, alkylamino, heteroarylamino, alkylcarbonylamino, alkylaminocarbonylamino, arylaminocarbonylamino, aralkylcarbonylamino, heteroarylcarbonylamino, arylcarbonylaamino, cycloamidino, cycloalkyl, cycloimido, arylsulfonyl and arylsulfonamido.

Lactam compounds as described throughout this application include compounds of formula (VIII) as shown below: 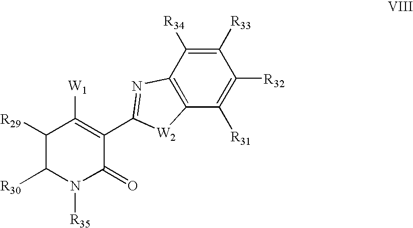
wherein,
W₁ is selected from the group consisting of —OH, —OR₃₆ groups, —NR₃₇R₃₈;
W₂ is selected from the group consisting of O, S, and NR₃₉ groups;
R₂₉ and R₃₀ join to form a 5 to 6 membered substituted or unsubstituted ring comprising all carbon atoms or at least one O, N, or S atom;
R₃₅ and R₃₉ may be the same or different and are selected from the group consisting of H, —OH substituted and unsubstituted alkyl groups, substituted and unsubstituted aryl groups, —C(═O)H, —C(═O)-alkyl groups, and —C(═O)-aryl groups;
R₃₁, R₃₂, R₃₃, and R₃₄ may be the same or different and are independently selected from the group consisting of H, Cl, Br, F, I, —NO₂, —CN, —OH, —OR₄₀ groups, —NR₄₁R₄₂ groups, —C(═O)R₄₃ groups, —SH groups, substituted and unsubstituted amidinyl groups, substituted and unsubstituted guanidinyl groups, substituted and unsubstituted alkyl groups, substituted and unsubstituted aryl groups, substituted and unsubstituted alkenyl groups, substituted and unsubstituted alkynyl groups, substituted and unsubstituted heterocyclyl groups, substituted and unsubstituted alkylaminoalkyl groups, substituted and unsubstituted dialkylaminoalkyl groups, substituted and unsubstituted arylaminoalkyl groups, substituted and unsubstituted diarylaminoalkyl groups, substituted and unsubstituted (alkyl)(aryl)aminoalkyl groups, substituted and unsubstituted heterocyclylalkyl groups, substituted and unsubstituted aminoalkyl groups, substituted and unsubstituted heterocyclylaminoalkyl groups, substituted and unsubstituted diheterocyclylaminoalkyl groups, substituted and unsubstituted (alkyl)(heterocyclyl)aminoalkyl groups, substituted and unsubstituted (aryl)(heterocyclyl)aminoalkyl groups, substituted and unsubstituted hydroxyalkyl groups, substituted and unsubstituted alkoxyalkyl groups, substituted and unsubstituted aryloxyalkyl groups, and substituted and unsubstituted heterocyclyloxyalkyl groups;
R₃₆ is selected from the group consisting of substituted and unsubstituted alkyl groups, substituted and unsubstituted aryl groups, substituted and unsubstituted heterocyclyl groups, substituted and unsubstituted heterocyclylalkyl groups, —C(═O)H, —C(═O)-alkyl groups, —C(═O)-aryl groups, —C(═O)O-alkyl groups, —C(═O)O-aryl groups, —C(═O)NH₂, —C(═O)NH(alkyl) groups, —C(═O)NH(aryl) groups, —C(═O)N(alkyl)₂ groups, —C(═O)N(aryl)₂ groups, —C(═O)N(alkyl)(aryl) groups, —NH₂, —NH(alkyl) groups, —NH(aryl) groups, —N(alkyl)₂ groups, —N(alkyl)(aryl) groups, —N(aryl)₂ groups, —C(═O)NH(heterocyclyl) groups, —C(═O)N(heterocyclyl)₂ groups, —C(═O)N(alkyl)(heterocyclyl) groups, and —C(═O)N(aryl)(heterocyclyl) groups;
R₃₇ is selected from the group consisting of H, substituted and unsubstituted alkyl groups, substituted and unsubstituted aryl groups, and substituted and unsubstituted heterocyclyl groups;
R₃₈ is selected from the group consisting of H, substituted and unsubstituted alkyl groups, substituted and unsubstituted aryl groups, substituted and unsubstituted heterocyclyl groups, —OH, alkoxy groups, aryloxy groups, —NH₂, substituted and unsubstituted heterocyclylalkyl groups, substituted and unsubstituted aminoalkyl groups, substituted and unsubstituted alkylaminoalkyl groups, substituted and unsubstituted dialkylaminoalkyl groups, substituted and unsubstituted arylaminoalkyl groups, substituted and unsubstituted diarylaminoalkyl groups, substituted and unsubstituted (alkyl)(aryl)aminoalkyl groups, substituted and unsubstituted alkylamino groups, substituted and unsubstituted arylamino groups, substituted and unsubstituted dialkylamino groups, substituted and unsubstituted diarylamino groups, substituted and unsubstituted (alkyl)(aryl)amino groups, —C(═O)H, —C(═O)-alkyl groups, —C(═O)-aryl groups, —C(═O)O-alkyl groups, —C(═O)O-aryl groups, —C(═O)NH₂, —C(═O)NH(alkyl) groups, —C(═O)NH(aryl) groups, —C(═O)N(alkyl)₂ groups, —C(═O)N(aryl)₂ groups, —C(═O)N(alkyl)(aryl) groups, —C(═O)-heterocyclyl groups, —C(═O)—O-heterocyclyl groups, —C(═O)NH(heterocyclyl) groups, —C(═O)-N(heterocyclyl)₂ groups, —C(═O)-N(alkyl)(heterocyclyl) groups, —C(═O)-N(aryl)(heterocyclyl) groups, substituted and unsubstituted heterocyclylaminoalkyl groups, substituted and unsubstituted diheterocyclylaminoalkyl groups, substituted and unsubstituted (alkyl)(heterocyclyl)aminoalkyl groups, substituted and unsubstituted (aryl)(heterocyclyl)aminoalkyl groups, substituted and unsubstituted hydroxyalkyl groups, substituted and unsubstituted alkoxyalkyl groups, substituted and unsubstituted aryloxyalkyl groups, and substituted and unsubstituted heterocyclyloxyalkyl groups;
R₄₁ is selected from the group consisting of H, substituted and unsubstituted alkyl groups, substituted and unsubstituted aryl groups, and substituted and unsubstituted heterocyclyl groups;
R₄₂ is selected from the group consisting of H, substituted and unsubstituted alkyl groups, substituted and unsubstituted aryl groups, substituted and unsubstituted heterocyclyl groups, —C(═O)H, —C(═O)-alkyl groups, —C(═O)-aryl groups, —C(═O)NH₂, —C(═O)NH(alkyl) groups, —C(═O)NH(aryl) groups, —C(═O)N(alkyl)₂ groups, —C(═O)N(aryl)₂ groups, —C(═O)N(alkyl)(aryl) groups, —C(═O)O-alkyl groups, —C(═O)O-aryl groups, substituted and unsubstituted aminoalkyl groups, substituted and unsubstituted alkylaminoalkyl groups, substituted and unsubstituted dialkylaminoalkyl groups, substituted and unsubstituted arylaminoalkyl groups, substituted and unsubstituted diarylaminoalkyl groups, substituted and unsubstituted (alkyl)(aryl)aminoalkyl groups, substituted and unsubstituted heterocyclylalkyl groups, —C(═O)-heterocyclyl groups, —C(═O)—O-heterocyclyl groups, —C(═O)NH(heterocyclyl) groups, —C(═O)—N(heterocyclyl)₂ groups, —C(═O)—N(alkyl)(heterocyclyl) groups, —C(═O)—N(aryl)(heterocyclyl) groups, substituted and unsubstituted heterocyclylaminoalkyl groups, substituted and unsubstituted diheterocyclylaminoalkyl groups, substituted and unsubstituted (heterocyclyl)(alkyl)aminoalkyl groups, substituted and unsubstituted (heterocyclyl)(aryl)aminoalkyl groups, substituted and unsubstituted hydroxyalkyl groups, substituted and unsubstituted alkoxyalkyl groups, substituted and unsubstituted aryloxyalkyl groups, and substituted and unsubstituted heterocyclyloxyalkyl groups; and
R₄₃ is selected from the group consisting of H, —NH₂, —NH(alkyl) groups, —NH(aryl) groups, —N(alkyl)₂ groups, —N(aryl)₂ groups, —N(alkyl)(aryl) groups, —NH(heterocyclyl) groups, —N(heterocyclyl)(alkyl) groups, —N(heterocyclyl)(aryl) groups, —N(heterocyclyl)₂ groups, substituted and unsubstituted alkyl groups, substituted and unsubstituted aryl groups, —OH, substituted and unsubstituted alkoxy groups, substituted and unsubstituted heterocyclyl groups, substituted and unsubstituted aryloxy groups, heterocyclyloxy groups, —NHOH, —N(alkyl)OH groups, —N(aryl)OH groups, —N(alkyl)O-alkyl groups, —N(aryl)O-alkyl groups, —N(alkyl)O-aryl groups, and —N(aryl)O-aryl groups.

Preferably R₂₉ and R₃₀ join together to form a substituted or unsubstituted phenyl ring.

Hydropthalamide compounds as described throughout this application include compounds of formula (IX) as shown below: 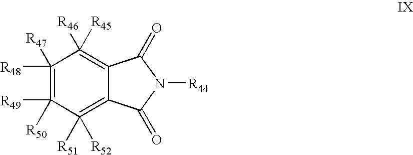
wherein,
R₄₄ is selected from the group consisting of substituted or unsubstituted aryl, heteroaryl, arylalkyl, heteroarylalkyl, fused arylaryl, unfused arylaryl, fused heteroarylaryl, unfused heteroarylaryl, fused arylheteroaryl, and unfused arylheteroaryl;
R₄₅, R₄₇, R₄₉, and R₅₁ may be the same or different and are independently selected from the group consisting of H, nitro, halogen, amino, hydroxy, cyano, carboxcyclic acid, and substituted or unsubstituted alkyl, aryl, heteroaryl, alkoxy, alkylcarbonyl, alkylcarbonylamino, alkylaminocarbonyl, aminocarbonyl, arylalkoxy, heteroarylalkoxy, alkylamino, arylalkylamino, arylamino, heteroarylamino, heteroarylaminoalkyl, heterocyclyl, heterocyclylalkoxy, heterocyclylalkyl, and carbocyclyl; and
R₄₆, R₄₈, R₅₀, and R₅₂ may be the same or different and are independently selected from the group consisting of H, halogen, and substituted or unsubstituted alkyl groups.

Benzophenone compounds as described throughout this application include compounds of formula (X) as shown below: 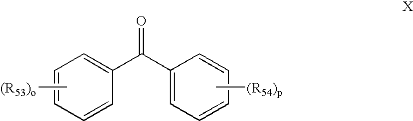
wherein,
R₅₃ is independently selected from the group consisting of H, nitro, halogen, amino, hydroxy, cyano, carboxcyclic acid, and substituted or unsubstituted alkyl, aryl, heteroaryl, alkoxy, alkylcarbonyl, alkylcarbonylamino, alkylaminocarbonyl, aminocarbonyl, arylalkoxy, heteroarylalkoxy, alkylamino, arylalkylamino, arylamino, heteroarylamino, heteroarylaminoalkyl, heterocyclyl, heterocyclylalkoxy, heterocyclylalkyl, and carbocyclyl;
R₅₄ is independently selected from the group consisting of H, nitro, halogen, amino, hydroxy, cyano, carboxcyclic acid, and substituted or unsubstituted alkyl, aryl, heteroaryl, alkoxy, alkylcarbonyl, alkylcarbonylamino, alkylaminocarbonyl, aminocarbonyl, arylalkoxy, heteroarylalkoxy, alkylamino, arylalkylamino, arylamino, heteroarylamino, heteroarylaminoalkyl, heterocyclyl, heterocyclylalkoxy, heterocyclylalkyl, and carbocyclyl; and
o and p are integers from 0-4.

Isoxazole compounds as described throughout this application include compounds of formula (XI) as shown below: 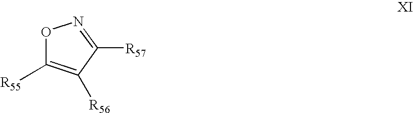
wherein,
R₅₅ is selected from the group consisting of substituted or unsubstituted aryl, arylalkyl, heteroaryl, heteroarylalkyl, heterocyclyl, and heterocyclylalkyl;
R₅₆ is selected from the group consisting of substituted or unsubstituted aryl, arylalkyl, heteroaryl, heteroarylalkyl, heterocyclyl, and heterocyclylalkyl; and,
R₅₇ is selected from the group consisting of H, halogen, hydoxy, and substituted or unsubstituted alkyl, aryl, heteroaryl, heterocyclyl, and carbonyl.

Sterol compounds as described throughout this application include compounds of formula (XII) as shown below: 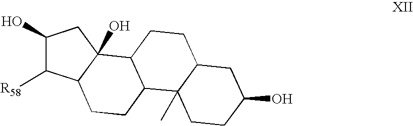
wherein,
R₅₈ is selected from the group consisting of substituted or unsubstituted aryl, arylalkyl, heteroaryl, heteroarylalkyl, heterocyclyl, and heterocyclylalkyl.

Preferably R₅₈ is a pyranone substituent.

Quinazilinone compounds as described throughout this application include compounds of formula (XIII) as shown below: 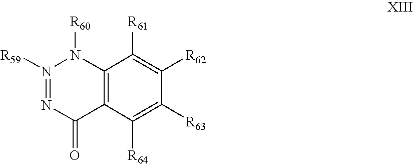
wherein,
R₅₉ is selected from the group consisting of H, halogen, hydroxy, and substituted or unsubstituted alkyl, aminoalkyl, alklyaminoalkyl, alkoxy, dialkylaminoalkyl, hydroxyalkyl, alkenyl, alkynyl, carbocyclyl, carbocyclylalkyl, heterocyclyl, and heterocyclylalkyl;
R₆₀ is selected from the group consisting of substituted or unsubstituted aryl, heteroaryl, arylalkyl, heteroarylalkyl, and heterocyclylalkyl; and,
R₆₁, R₆₂, R₆₃, and R₆₄ may be the same or different and are independently selected from the group consisting of H, nitro, halogen, amino, hydroxy, cyano, carboxcyclic acid, and substituted or unsubstituted alkyl, aryl, heteroaryl, alkoxy, alkylcarbonyl, alkylcarbonylamino, alkylaminocarbonyl, aminocarbonyl, arylalkoxy, heteroarylalkoxy, alkylamino, arylalkylamino, arylamino, heteroarylamino, heteroarylaminoalkyl, heterocyclyl, heterocyclylalkoxy, heterocyclylalkyl, and carbocyclyl groups.

Pyrrole compounds as described throughout this application include compounds of formula (XIV) as shown below: 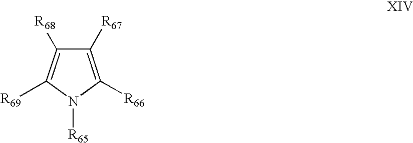
wherein,
R₆₅ is selected from the group consisting of H, hydroxy, and substituted or unsubstituted alkyl, aryl, heteroaryl, heteroarylalkyl, arylalkyl, heteroarylaminoalkyl, arylaminoalkyl, heteroaryloxyalkyl, and aryloxyalkyl groups;
R₆₆, R₆₇, R₆₈, and R₆₉ may be the same or different and are independently selected from the group consisting of H, nitro, halogen, amino, hydroxy, cyano, carboxcyclic acid, and substituted or unsubstituted alkyl, aryl, heteroaryl, alkoxy, alkylcarbonyl, alkylcarbonylamino, alkylaminocarbonyl, aminocarbonyl, arylalkoxy, heteroarylalkoxy, alkylamino, arylalkylamino, arylamino, heteroarylamino, heteroarylaminoalkyl, heterocyclyl, heterocyclylalkoxy, heterocyclylalkyl, and carbocyclyl groups.

Further preferred pyrrole compounds include those shown in Formula (XV): 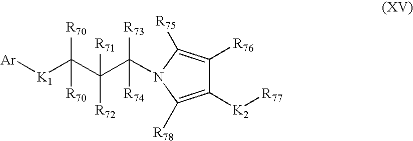
wherein:
K₁ is nitrogen, oxygen, or optionally substituted carbon;
W is absent or is selected from the group consisting of —O—, —S—, —S(O)—, —SO₂—, —NH—, —NH—CO—, —NR′CO—, —NHSO₂—, —NR′SO₂—, —CO—, —CO₂—, —CH₂—, —CF₂—, CHF, —CONH—, —CONR′—, and —NR′—, where R′ is alkyl, substituted alkyl, cycloalkyl, aryl, heteroaryl, heterocyclo; Ar is optionally substituted aryl, heteroaryl, or a protecting group;
R₇₀ and R₇₀′ are independently selected from the group consisting of hydrogen and methyl;
R₇₁, R₇₂, R₇₃, and R₇₄ are independently selected from the group consisting of hydrogen, hydroxyl, and optionally substituted loweralkyl, cycloloweralkyl, cyclicaminoalkyl, alkylaminoalkyl, loweralkoxy, amino, alkylamino, alkylcarbonyl, arylcarbonyl, aralkylcarbonyl, heteroarylcarbonyl, heteroaralkylcarbonyl, aryl and heteroaryl;
R₇₅ and R₇₈ are independently selected from the group consisting of hydrogen, halo, and optionally substituted loweralkyl, cycloalkyl, alkoxy, amino, aminoalkoxy, carbonyloxy, aminocarbonyloxy, alkylcarbonylamino, arylcarbonylamino, aralkylcarbonylamino, heteroarylcarbonylamino, heteroaralkylcarbonylamino, cycloimido, heterocycloimido, amidino, cycloamidino, heterocycloamidino, guanidinyl, aryl, heteroaryl, heterocycloalkyl, heterocyclocarbonyloxy, heteroarylcarbonyloxy, and arylsulfonamido;
R₇₆ is selected from the group consisting of hydrogen, aryl, heteroaryl, substituted heteroaryl, heterocyclyl, and substituted heterocyclyl;
R₇₇ is selected from the group consisting of hydrogen, hydroxy, halo, carboxyl, nitro, amino, amido, amidino, imido, cyano, sulfonyl, methanesulonyl, and substituted or unsubstituted alkyl, alkoxy, alkylcarbonyl, arylcarbonyl, aralkylcarbonyl, heteroarylcarbonyl, heteroaralkylcarbonyl, alkylcarbonyloxy, arylcarbonyloxy, aralkylcarbonyloxy, heteroarylcarbonyloxy, heteroaralkylcarbonyloxy, alkylaminocarbonyloxy, arylaminocarbonyloxy, formyl, loweralkylcarbonyl, loweralkoxycarbonyl, aminocarbonyl, aminoaryl, alkylsulfonyl, sulfonamido, aminoalkoxy, alkylamino, heteroarylamino, alkylcarbonylamino, alkylaminocarbonylamino, arylaminocarbonylamino, aralkylcarbonylamino, heteroarylcarbonylamino, arylcarbonylamino, heteroarylcarbonylamino cycloamido, cyclothioamido, cycloamidino, heterocycloamidino, cycloalkyl, cycloimido, heterocycloimido, guanidinyl, aryl, heteroaryl, heterocyclo, heterocycloalkyl, arylsulfonyl and arylsulfonamido;

Anthraquinone compounds of the instant invention include, for example, compounds of Formula (XVI): 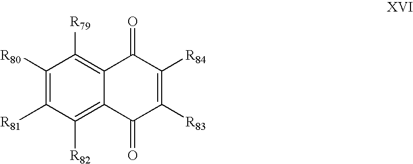
wherein,
R₇₉, R₈₀, R₈₁, and R₈₂ may be the same or different and are independently selected from the group consisting of H, nitro, halogen, amino, hydroxy, cyano, carboxcyclic acid, and substituted or unsubstituted alkyl, aryl, heteroaryl, alkoxy, alkylcarbonyl, alkylcarbonylamino, sulfonyl, aminosulfonyl, alkylaminocarbonyl, aminocarbonyl, arylalkoxy, heteroarylalkoxy, alkylamino, arylalkylamino, arylamino, heteroarylamino, heteroarylaminoalkyl, heterocyclyl, heterocyclylalkoxy, heterocyclylalkyl, and carbocyclyl groups; and,
R₈₃ and R₈₄ are taken together to form a substituted or unsubstituted 5-6 membered ring containing all carbon atoms or 1-2 heteroatoms selected from the group consisting of O, S, and N.

Quinoxaline compounds referred to throughout this application include tricyclic, partially unconjugated compounds optionally substituted with nitrogen heteroatoms as shown in the preferred quinoxaline embodiment (XVII) below: 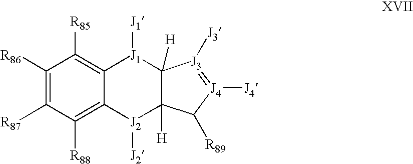
wherein,
J₁ is either C or N,
J₁′ is selected from the group consisting of H, substituted aryl, unsubstituted aryl, substituted heteroaryl, and unsubstituted heteroaryl;
J₂ is either C or N,
J₂′ is selected from the group consisting of H, substituted aryl, unsubstituted aryl, substituted heteroaryl, and unsubstituted heteroaryl;
J₃ is selected from the group consisting of —CO—, —NH—, and —N═;
if J₄ is —O— then J₄′ is absent; or,
if J₄ is ═C— then J₄′ is selected from the group consisting of H and substituted or unsubstituted alkyl, alkoxy, aryl, heteroaryl, heteroarylalkyl, arylalkyl, aminoalkyl, alkylamino, and alkylthio groups; and,
R₈₅, R₈₆, R₈₇, R₈₈, and R₈₉ may be the same or different and are independently selected from the group consisting of H, nitro, halogen, amino, hydroxy, cyano, carboxcyclic acid, and substituted or unsubstituted alkyl, aryl, heteroaryl, alkoxy, alkylcarbonyl, alkylcarbonylamino, sulfonyl, aminosulfonyl, alkylaminocarbonyl, aminocarbonyl, arylalkoxy, heteroarylalkoxy, alkylamino, arylalkylamino, arylamino, heteroarylamino, heteroarylaminoalkyl, heterocyclyl, heterocyclylalkoxy, heterocyclylalkyl, and carbocyclyl groups.

Triazine compounds refer to substituted 6-membered heterocyclic groups with 3 nitrogen atoms distributed throughout the ring. The preferred embodiments of the instant invention include those shown in structures (XVIII), (XIX) and (XX) shown below: 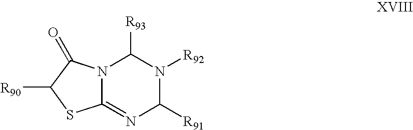
wherein,
R₉₀ is selected from the group consisting of substituted or unsubstituted alkyl, alkenyl, akynyl, aryl, heteroaryl, heteroarylalkyl, heteroarylalkenyl, arylalkyl, and arylalkenyl;
R₉₁ and R₉₃ are independently selected from the group consisting of H, and unsubstituted alkyl;
R₉₁ is aryl; preferably phenyl, 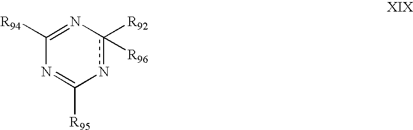
   wherein,
R₉₄ is selected from the group consisting of H, amino, alkyl, aminoalkyl, and halogen;
R₉₅ is selected from the group consisting of substituted or unsubstituted aryl, arylamino, arylalkylamino, heteroaryl, heteroarylamino, and heteroalkylamino;
R₉₆ and R₉₇ are independently selected from the group consisting of H, halogen, and alkyl, preferably methyl; or,
R₉₆ may form a double bond with the nitrogen atom directly below it as indicated by the dashed line in the above structure; and, 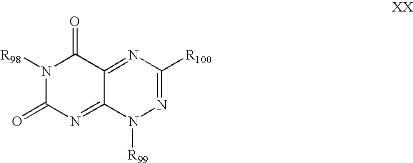
   wherein,

R₉₈ is selected from the group consisting of H, substituted alkyl, and unsubstituted alkyl; preferably methyl,
R₉₉ is selected from the group consisting of H, substituted alkyl, and unsubstituted alkyl; preferably ethyl,
R₁₀₀ is selected from the group consisting of substituted or unsubstituted aryl, heteroaryl, alkoxyaryl, arylalkyl, and heteroarylalkyl.

Benzazole compounds as described throughout this application include compounds of formula (XXI) as shown below: 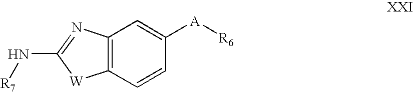
wherein,
A is selected from the group consisting of —O—, —S—, —NH—, and —NR₈—;
W is selected from the group consisting of —CH₂—, —O—, —S—, —NH—, and —NR₈—;
R₇ is selected from the group consisting of carbocyclyl, unfused carbocyclylcarbocyclyl, substituted aryl, unsubstituted aryl, substituted heteroaryl, unsubstituted heteroaryl, substituted fused arylheteroaryl, unsubstituted fused arylheteroaryl, substituted unfused arylaryl and unsubstituted unfused arylaryl;
R₆ is selected from the group consisting of substituted or unsubstituted aryl, and heteroaryl; and,
R₈ is independently substituted or unsubstituted alkyl.

Pyrazalopyrimidine compounds as described throughout this application include compounds of formula (XXII) as shown below: 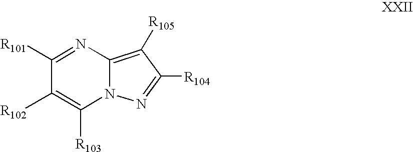
wherein,
R₁₀₁ is selected from the group consisting of H, nitro, halogen, amino, hydroxy, cyano, carboxcyclic acid, and substituted or unsubstituted alkyl, aryl, heteroaryl, alkoxy, alkylcarbonyl, alkylcarbonylamino, sulfonyl, aminosulfonyl, alkylaminocarbonyl, aminocarbonyl, arylalkoxy, heteroarylalkoxy, alkylamino, arylalkylamino, arylamino, heteroarylamino, heteroarylaminoalkyl, heterocyclyl, heterocyclylalkoxy, heterocyclylalkyl, and carbocyclyl groups;
R₁₀₂ is selected from the group consisting of H, nitro, halogen, amino, hydroxy, cyano, carboxcyclic acid, and substituted or unsubstituted alkyl, aryl, heteroaryl, alkoxy, alkylcarbonyl, alkylcarbonylamino, alkylaminocarbonyl, aminocarbonyl, arylalkoxy, heteroarylalkoxy, alkylamino, arylalkylamino, arylamino, heteroarylamino, heteroarylaminoalkyl, heterocyclyl, heterocyclylalkoxy, heterocyclylalkyl, and carbocyclyl groups;
R₁₀₃ is selected from the group consisting of H, nitro, halogen, amino, hydroxy, cyano, carboxcyclic acid, trifluoromethyl, and substituted or unsubstituted alkyl, aryl, heteroaryl, alkoxy, alkylcarbonyl, alkylcarbonylamino, alkylaminocarbonyl, aminocarbonyl, arylalkoxy, heteroarylalkoxy, alkylamino, arylalkylamino, arylamino, heteroarylamino, heteroarylaminoalkyl, heterocyclyl, heterocyclylalkoxy, heterocyclylalkyl, and carbocyclyl groups;
R₁₀₄ is selected from the group consisting of H and substituted or unsubstituted aryl, heteroaryl, arylalkoxy, heteroarylalkoxy, arylalkylamino, arylamino, heteroarylamino, heteroarylaminoalkyl, heterocyclyl, heterocyclylalkoxy, heterocyclylalkyl, carbocyclylalkyl and carbocyclyl groups;
R₁₀₅ is selected from the group consisting of H and substituted or unsubstituted aryl, heteroaryl, arylalkoxy, heteroarylalkoxy, arylalkylamino, arylamino, heteroarylamino, heteroarylaminoalkyl, heterocyclyl, heterocyclylalkoxy, heterocyclylalkyl, carbocyclylalkyl and carbocyclyl groups;
wherein at least one of R₁₀₄ and R₁₀₅ is not H.

SMIP compounds identified by in-vitro (cellular or non-cellular assays) or in-vivo methods are thoroughly described in Methods 1 and 2 below.

Pharmaceutical compositions containing the compounds of the invention may be in any form suitable for the intended method of administration, including, for example, a solution, a suspension, or an emulsion. Liquid carriers are typically used in preparing solutions, suspensions, and emulsions. Liquid carriers contemplated for use in the practice of the present invention include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, coconut oil, olive oil, safflower oil, cottonseed oil, and the like. For parenteral administration, the carrier can also be an oily ester such as ethyl oleate, isopropyl myristate, and the like. Compositions of the present invention may also be in the form of microparticles, microcapsules, liposomal encapsulates, and the like, as well as combinations of any two or more thereof.

Other additives include immunostimulatory agents known in the art. Immunostimulatory oligonucleotides and polynucleotides are described in PCT WO 98/55495 and PCT WO 98/16247. U.S. Patent Application No. 2002/0164341 describes adjuvants including an unmethylated CpG dinucleotide (CpG ODN) and a non-nucleic acid adjuvant. U.S. Patent Application No. 2002/0197269 describes compositions comprising an antigen, an antigenic CpG-ODN and a polycationic polymer. Other immunostimulatory additives described in the art may be used, for example, as described in U.S. Pat. No. 5,026,546; U.S. Pat. No. 4,806,352; and U.S. Pat. No. 5,026,543.

A controlled release delivery system may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York 1992. The matrix may be, for example, a biodegradable material that can degrade spontaneously in situ and in vivo for, example, by hydrolysis or enzymatic cleavage, e.g., by proteases. The delivery system may be, for example, a naturally occurring or synthetic polymer or copolymer, for example in the form of a hydrogel. Exemplary polymers with cleavable linkages include polyesters, polyorthoesters, polyanhydrides, polysaccharides, poly(phosphoesters), polyamides, polyurethanes, poly(imidocarbonates) and poly(phosphazenes).

The compounds of the invention may be administered enterally, orally, parenterally, sublingually, by inhalation spray, rectally, or topically in dosage unit formulations containing conventional nontoxic pharmaceutically acceptable carriers, adjuvants, and vehicles as desired. For example, suitable modes of administration include oral, subcutaneous, transdermal, transmucosal, iontophoretic, intravenous, intramuscular, intraperitoneal, intranasal, subdermal, rectal, and the like. Topical administration may also involve the use of transdermal administration such as transdermal patches or ionophoresis devices. The term parenteral includes subcutaneous injections, intravenous, intramuscular, intrastemal injection, or infusion techniques.

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-propanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Suppositories for rectal administration of the drug can be prepared by mixing the drug with a suitable nonirritating excipient such as cocoa butter and polyethylene glycols that are solid at ordinary temperatures but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose lactose or starch. Such dosage forms may also comprise, as is normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

As to the mode of administration, it should be emphasized that it is the combination of therapeutic agents that gives rise to its synergistic therapeutic effect no matter whether the first and the second agent are administered together or separately. Therefore, the two agents may be given together in a single dose or in separate ones with respect to space and time.

Effective amounts of the compounds of the invention generally include any amount sufficient to detectably treat viral infections.

Successful treatment of a subject in accordance with the invention may result in the inducement of a reduction or alleviation of symptoms in a subject afflicted with a medical or biological disorder to, for example, halt the further progression of the disorder, or the prevention of the disorder.

The amount of active ingredient that may be combined with the carrier materials to produce a single dosage form will vary depending upon the host treated and the particular mode of administration. It will be understood, however, that the specific dose level for any particular patient will depend upon a variety of factors including the activity of the specific compound employed, the age, body weight, general health, sex, diet, time of administration, route of administration, rate of excretion, drug combination, and the severity of the particular disease undergoing therapy. The therapeutically effective amount for a given situation can be readily determined by routine experimentation and is within the skill and judgment of the ordinary clinician.

The compounds of the present invention can also be administered in the form of liposomes. As is known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

While the SMIP compounds of the invention can be administered as the sole active pharmaceutical agent, they can also be used in combination with one or more other agents used in the treatment of SARSs. Other representative agents useful in combination with the compounds of the invention for the treatment of viral infections include, for example, interferon, ribavirin, gancyclovir and the like.

When additional active agents are used in combination with the compounds of the present invention, the additional active agents may generally be employed in therapeutic amounts as indicated in the PHYSICIANS' DESK REFERENCE (PDR) 53^{rd} Edition (1999), that is incorporated herein by reference, or such therapeutically useful amounts as would be known to one of ordinary skill in the art.

The compounds of the invention and the other therapeutically active agents can be administered at the recommended maximum clinical dosage or at lower doses. Dosage levels of the active compounds in the compositions of the invention may be varied so as to obtain a desired therapeutic response depending on the route of administration, severity of the disease and the response of the patient. The combination can be administered as separate compositions or as a single dosage form containing both agents. When administered as a combination, the therapeutic agents can be formulated as separate compositions that are given at the same time or different times, or the therapeutic agents can be given as a single composition.

Compounds of the present invention can be readily synthesized using the methods described herein, or other methods, that are well known in the art.

The compounds can be used in the form of salts derived from inorganic or organic acids. These salts include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxyethanesulfonate, lactate, maleate, methanesulfonate, nicotinate, 2-napthalenesulfonate, oxalate, pamoate, pectinate, persulfate, 3-phenylproionate, picrate, pivalate, propionate, succinate, tartrate, thiocyanate, p-toluenesulfonate and undecanoate. Also, the basic nitrogen-containing groups can be quaternized with such agents as loweralkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides, and others. Water or oil-soluble or dispersible products are thereby obtained.

Examples of acids that may be employed to form pharmaceutically acceptable acid addition salts include such inorganic acids as hydrochloric acid, sulphuric acid and phosphoric acid and such organic acids as oxalic acid, maleic acid, succinic acid and citric acid. Basic addition salts can be prepared in situ during the final isolation and purification of the compounds of formula (I), or separately by reacting carboxylic acid moieties with a suitable base such as the hydroxide, carbonate or bicarbonate of a pharmaceutical acceptable metal cation or with ammonia, or an organic primary, secondary or tertiary amine. Pharmaceutical acceptable salts include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine and the like.

Various compounds and methods of their synthesis are disclosed in international patent application Publication Nos. WO02/18327 (benzamide and pyridylamide based compounds); WO0222598, and WO02/18383 (ABIQ based compounds); and WO 02/81443 (pthalamide base compounds), that have been found within context of this invention to be useful for immune potentiation. The entire disclosure of these U.S. and international publications is incorporated herein by this reference. Other compounds or intermediates of interest in the present invention were purchased from commercially available sources using the following method: the chemical structure of interest was drawn into the ACD-SC database (from MDL Information Systems). A search of the following companies/institutions, among others, retrieved the identified compound's supplier and purchasing information: ASDI, ASINEX, BIONET, CHEMBRIDGE, CHEMDIV, CHEMEX, CHEMSTAR, COMGENEX, CSC, INTERBIOSCREEN, LABOTEST, MAYBRIDGE, MICROSOURCE/GENESIS, OLIVIA, ORION, PEAKDALE, RYAN SCIENTIFIC, SPECS, TIMTEC, U OF FLORIDA, and ZELINSKY.

Benzazle Compounds

### Scheme 1

Compounds of the invention containing a benzimidazole core may be prepared using a number of methods familiar to one of skill in the art. In one method, suitably functionalized diamines may be coupled with various thioisocyanates to form the intermediate thioureas. Cyclization to form the benzimidazole moiety may be effected under known conditions such as with treatment carbodiimides or alkyl halides. Alternatively the diamines may be reacted sequentially with carbonyl diimidazole and phosphoryl chloride followed by coupling with the appropriate amine.

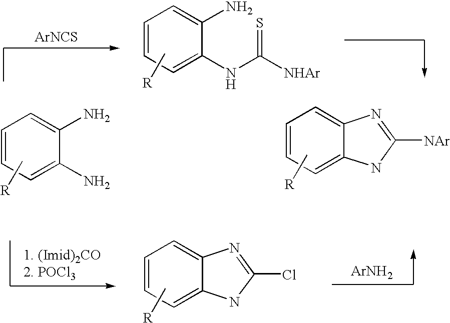

Compounds containing the oxazole structure may similarly be prepared according to the methods above or according to other known general procedures. Haviv et. al. (J. Med. Chem. 1988, 31, 1719) describes a procedure for assembling oxazole cores wherein a hydroxy aniline is treated with ethyl potassium xanthate. The resulting sulfuryl benzoxazole may then be chlorinated and coupled with an amine.

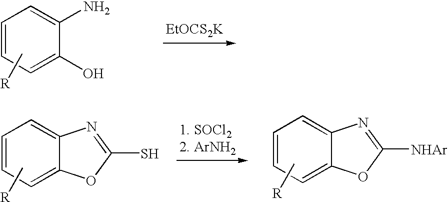

Compounds containing a benzothiazole core may also be prepared according to known methods. An ortho-halothioisocyanate may be reacted with an amine to form a thiourea. Reduction with NaH then allows formation of the thiazole ring.

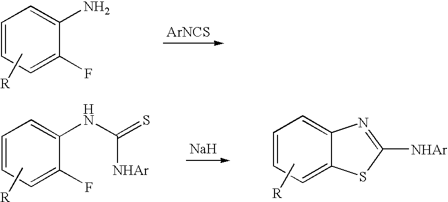

Benzothiazoles may generally be substituted in accordance with the present invention, such as through the following synthetic pathway:

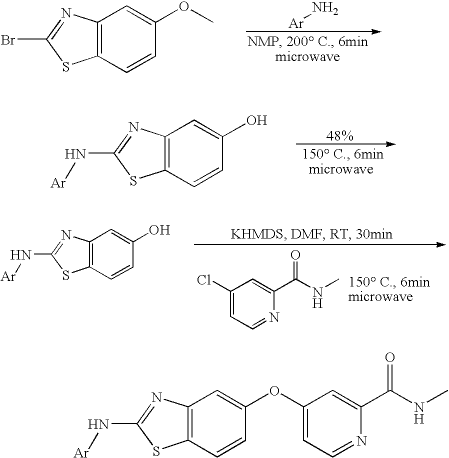

### Synthesis of 4-[(2-{[4-chloro-3-(trifluoromethyl)phenyl]amino}-1H-benzimidazol-6-yl)oxyl-N-methylpyridine-2-carboxamide

The compound 4-[(2-{[4-chloro-3-(trifluoromethyl)phenyl]amino}-1H-benzimidazol-6-yl)oxy]-N-methylpyridine-2-carboxamide (159322) was synthesized as follows:

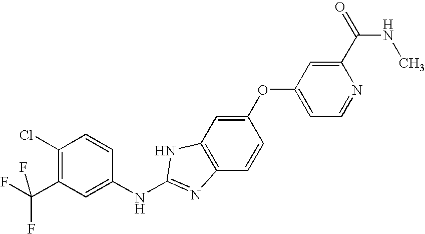

Step 1. Synthesis of 4-[(4-amino-3-nitrophenyl)oxy]-N-methylpyridine-2-carboxamide: A mixture containing 4-amino-3-nitrophenol (1 eq) and potassium bis(trimethylsilyl)amide (2 eq) was stirred in dimethylformamide for 2 hours at room temperature. To this mixture was added (4-chloro(2-pyridyl))-N-methylcarboxamide (1 eq) and potassium carbonate (1.2eq) and stirred at 90° C. for 3 days. The reaction mixture was then concentrated and partitioned between ethyl acetate and water. The organic layer was separated and washed with brine, dried, filtered, and concentrated in vacuum to give brown solid. Purification on silica gel (2% triethyl amine/50% ethyl acetate in hexane) gave 4-[(4-amino-3-nitrophenyl)oxy]-N-methylpyridine-2-carboxamide as an orange solid. The product gave satisfactory NMR. HPLC, 3.39 min; MS: MH⁺=289.

Step 2. Synthesis of 4-[(3,4-diaminophenyl)oxy]-N-methylpyridine-2-carboxamide: The mixture containing [4-(3-amino-4-nitrophenoxy)(2-pyridyl)]-N- in methanol with catalytic amount of 10% Pd/C was hydrogenated until disappearance of the yellow color to yield the product amine. HPLC, 2.5 mins; MS: MH⁺=259.

Step 3. Synthesis of 4-[(2-{[4-chloro-3-(trifluoromethyl)phenyl]amino}-1H-benzimidazol-6-yl)oxy]-N-methylpyridine-2-carboxamide: The mixture containing 4-[(3,4-diaminophenyl)oxy]-N-methylpyridine-2-carboxamide (1 eq) and 4-chloro-3-(trifluoromethyl)benzeneisothiocyanate (1 eq) in tetrahydrofuran was stirred at room temperature for 16 hours to give the corresponding thiourea. To the resulting mixture was added 1-ethyl-3-(3-dimethylaminopropyl)carbodiimide hydrochloride (2 eq) and the mixture was stirred for another 10 hours. The mixture was concentrated and partitioned between ethyl acetate and water. The organic layer was washed with brine and dried. Purification on HPLC gave 4-[(2-{[4-chloro-3-(trifluoromethyl)phenyl]amino}-1H-benzimidazol-6-yl)oxy]-N-methylpyridine-2-carboxamide. MS: MH⁺=462

### Synthesis of 4-({2-[(4-bromophenyl)amino]-1-methyl-1H-benzimidazol-5-yl}oxy)-N-methylpyridine-2-carboxamide

The compound 4-({2-[(4-bromophenyl)amino]-1-methyl-1H-benzimidazol-5-yl}oxy)-N-methylpyridine-2-carboxamide (161651) was synthesized as follows:

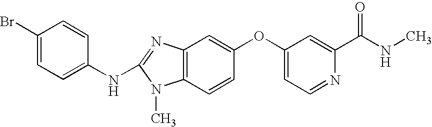

Step 1. Synthesis of 4-{[3-amino-4-(methylamino)phenyl]oxy}-N-methylpyridine-2-carboxamide: A solution of 4-[(4-amino-3-nitrophenyl)oxy]-N-methylpyridine-2-carboxamide (1 eq) in methylene chloride was treated with trifluoroacetic anhydride (I eq) and stirred for 10 minutes at 0° C. The mixture was quenched with satd. NaHCO₃ solution. The organic layer was separated and washed with water, brine, dried and evaporated. MS: MH⁺=385.2

To a solution of the trifluroacetamide (1 eq) in a mixture of toluene, acetonitrile and sodium hydroxide solution (50%) was added benzyltrimethylammonium chloride (1 eq) and dimethyl sulfate (1.2 eq). The biphasic mixture was stirred overnight at room temperature and evaporated. The mixture was taken up in ethyl acetate, washed with water, brine, dried and evaporated. The crude product was purified by column chromatography eluting with 1:1 hexanes and ethylacetate followed by 2% triethylamine in 1:1 hexanes and ethyl acetate followed by 2% triethylamine in 1:1 hexanes and ethyl acetate to afford N-methyl-4-{[4-(methylamino)-3-nitrophenyl]oxy}pyridine-2-carboxamide as a reddish orange solid. MS: MH⁺=303.1.

The solution of nitromethylaniline in methanol was treated with 5% palladium on carbon and stirred under hydrogen atmosphere for 15 min. (until the disappearance of yellow coloration) at room temperature. The mixture was filtered and the filtrate was concentrated to provide 0.36 g of the diamine 4-{[3-amino-4-(methylamino)phenyl]oxy}-N-methylpyridine-2-carboxamide. MS: MH⁺=273.3.

Step 2. Synthesis of 4-({2-[(4-bromophenyl)amino]-1-methyl-1H-benzimidazol-5-yl}oxy)-N-methylpyridine-2-carboxamide: A solution of the diamine 4-{[3-amino-4-(methylamino)phenyl]oxy}-N-methylpyridine-2-carboxamide (1 eq) in methanol was treated with 4-bromophenylisothiocyanate (1 eq) and stirred at 60° C.-65° C. for 2 hours. The reaction mixture was cooled down to room temperature and methyl iodide (1 eq) was added and stirred overnight at 60° C. The reaction was cooled to room temperature, evaporated, taken up in ethyl acetate, and washed with water and brine, dried, and evaporated under reduced pressure. Column chromatography using a gradient solvent system of hexanes and ethyl acetate and either 1:1 methylene chloride and acetone or 5% methanol in methylene chloride yielded the product as a half white powder. MS: MH⁺=452.3

Aminobenzimidazolylquinolinones

Compounds of structure I may be synthesized from simple starting molecules as shown in Schemes 1-4 and exemplified in the Examples. As shown in Scheme 1, compounds of structure I may generally be prepared using aromatic compounds substituted with amines and carboxylic acid groups.

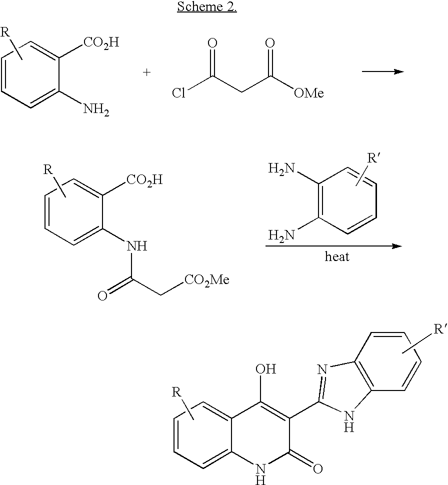

As shown in Scheme 2, a substituted aromatic compound such as a substituted or unsubstituted 2-aminobenzoic acid may be reacted with an acyl halide such as methyl 2-(chlorocarbonyl)acetate to produce an amide that will react with a substituted or unsubstituted 1,2-diaminobenzene. The resulting product is a 4-hydroxy-substituted compound of structure I. One skilled in the art will recognize that the procedure set forth in Scheme 1 may be modified to produce various compounds.

A method for preparing 4-amino substituted compounds of structure I is shown in Scheme 3. As shown in Scheme 3, aromatic compounds substituted with amine and nitrile 20 groups may be used to synthesize 4-amino substituted compounds of structure I. A compound such as ethyl 2-cyanoacetate may be reacted with ethanol to produce ethyl 3-ethoxy-3-iminopropanoate hydrochloride. Subsequent reaction with a substituted or unsubstituted 1,2-phenylenediamine provides substituted or unsubstituted ethyl 2-benzimidazol-2-ylacetate. Reaction of a substituted or unsubstituted ethyl 2-benzimidazol-2-ylacetate with an aromatic compound having an amine and nitrile group such as substituted or unsubstituted 2-aminobenzonitrile with a base such as lithium bis(trimethylsilyl)amide or a Lewis acid such as tin tetrachloride provides the substituted or unsubstituted 4-amino substituted compound of structure I.

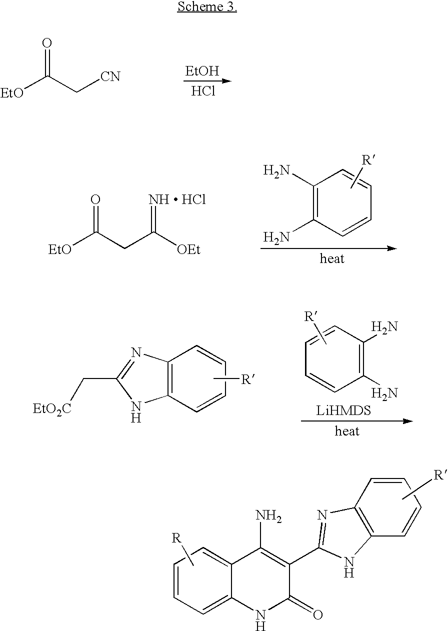

Scheme 4 illustrates a general synthetic route that allows for the synthesis of 4-dialkylamino and 4-alkylamino compounds of structure I. An inspection of Scheme 3 shows that 4-hydroxy substituted compounds of structure I may be converted into the 4-chloro derivative by reaction with phosphorous oxychloride or thionyl chloride. The 4-chloro derivative may then be reacted with an alkylamine or dialkylamine to produce the corresponding 4-alkylamino or 4-dialkylamino derivative. Deprotection affords the final 4-alkylamino or 4-dialkylamino compounds of structure I. Other groups that may be reacted with the 4-chloro derivative in this manner include, but are not limited to, ROH, RSH, and CuCN.

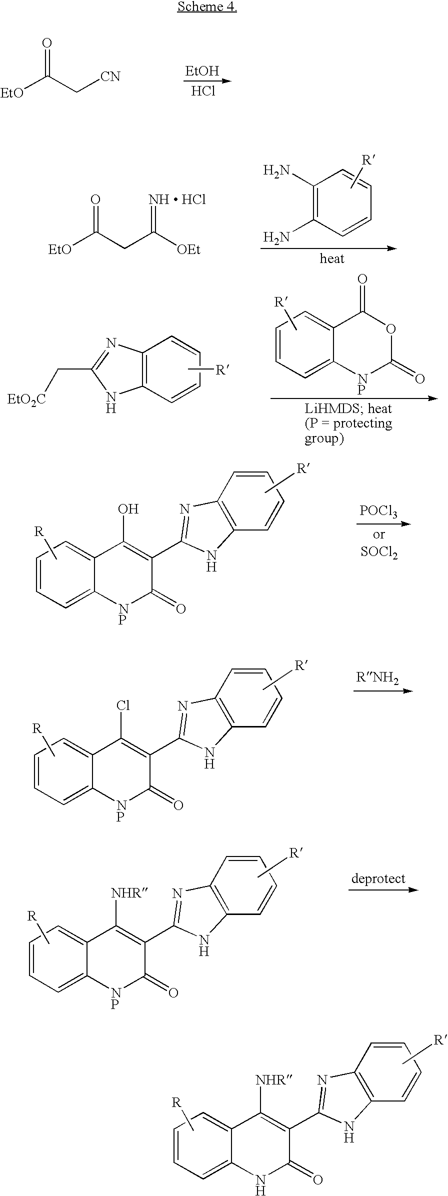

As shown in Scheme 5, the synthesis of compounds of structure I having a H, alkyl group, aryl group, or heterocyclyl group in the 4-position may be accomplished using a substituted or unsubstituted 2-benzimidazol-2-ylacetate prepared as shown in Schemes 3 and 4. 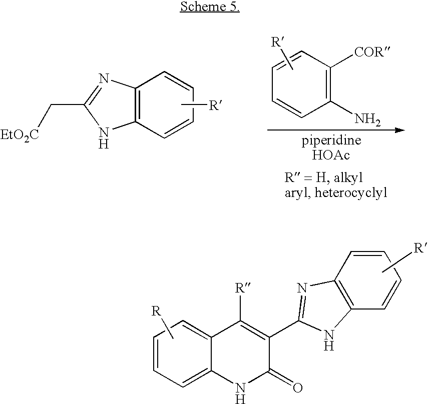
Thiosemcarbazones

General procedure for the preparation of thiosemicarbazones

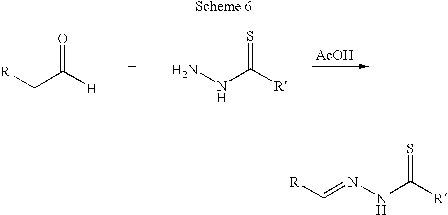

A solution of aldehyde (1.0 equiv.) and thiosemicarbazide (1.05 equiv.) in acetic acid was stirred overnight. Excess of acetic acid was removed to give a residue, that was washed with ethanol, or purified by preparative-HPLC to give the thiosemicarbazone.

### Scheme 7

A solution of aldehyde (1.0 equiv.), thiosemicarbazide (1.05 equiv.) and acetic acid (0.1 equiv.) in methanol was stirred overnight. Methanol was removed to give a residue, that was worked up as in Scheme 6.

### Scheme 8

To a solution of {[(1E)-1-aza-2-(4-fluoro-3-nitrophenyl)vinyl]aamino}-aminomethane-1-thione in ethanol was added an arylamine (2.1 equiv.). The solution was stirred at room temperature until the starting fluoride disappeared. The solution was purified to the product.

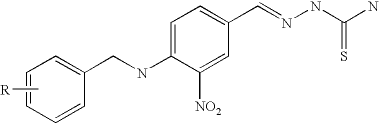

### Scheme 9

A mixture of 4-(diethylamino)-2-hydroxybenzaldehyde (1 equiv.), benzylic bromide (1.2 equiv.) and powder potassium carbonate in ethanol was stirred at room temperature for 2 days. Ethanol was removed, and the residue was dissolved in ethyl acetate and water. The organic layer was washed with aqueous NaHCO₃ and brine, dried over Na₂SO₄., and concentrated. The residue was purified on silica gel eluting with ethyl acetate/hexane to give 4-(diethylamino)-2-benzoxylic-benzaldehyde.

The aldehydes were converted to thiosemicarbazones according to Scheme 7.

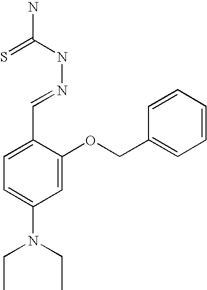

### Scheme 10

A solution of 3,4-difluorobenzenecarbonitrile (1 equiv.), amine (1.5 equiv.) and DIEA (2 equiv.) in NMP was heated in a Smith Microwave (Personal Chemistry) for 30 minutes. The reaction mixture was purified on silica gel to give 4-substituted 3-fluorobenzenecarbonitrile.

To a solution of nitrile in toluene at −78° C. was added DIBAL-H (1 M in toluene, 1.5 equiv.). The reaction mixture was warmed to rt, and stirred for 16 h, and quenched with methanol/ethyl acetate/brine (1:1:4). After being stirred at rt for 30 min, the solution was extracted with ethyl acetate (3×). The combined organic layers were washed with aqueous NaHCO₃, brine and concentrated. The aldehyde was purified on silica gel or directly converted to thiosemicarbazones (Scheme 7).

### Scheme 11

A solution of 2,4,5-trifluorobenzenecarbonitrile (1 equiv.) and 4-arylpiperazine (1.2 equiv.) and DIEA (1.2 equiv.) in THF was heated at 80° C. for 2 hours. The mixture was purified on silica gel to give 4-substituted 2,5-difluorobenzenecarbonitrile.

### Scheme 12

To an alcohol (1.0 equiv) was added potassium t-butoxide in THF (1 M, 1.1 equiv). After 5 minutes, the solution was added to a solution of 4-N-substituted-2,5-difluorobenzenecarbonitrile (1 equiv.) in THF. The reaction mixture was stirred at rt overnight and quenched with aqueous ammonium chloride. The aqueous layer was extracted with ethyl acetate (3×). The combined organic layers were washed with brine, and concentrated to give a residue, that was purified to give 4-N-substituted-2-O-substituted-5-fluorobenzenecarbonitrile.

4-N-substituted-2-O-substituted-5-fluorobenzenecarbonitrile was reduced with DIBAL-H to give a 4-N-substituted-2-O-substituted-5-fluorobenzaldehyde according to procedure in Scheme 10.

The aldehyde was converted to the corresponding thiosemicarbazone using Scheme 7.

### Scheme 13

A solution of 4-N-substituted-2,5-difluorobenzenecarbonitrile (1 equiv.), amine (1.5 equiv.) and DIEA (2 equiv.) in NMP was heated in a Smith Microwave (Personal Chemistry) for 30 minutes. The reaction mixture was purified on silica gel to give 4-N-substituted-2-N-substituted-5-fluorobenzenecarbonitrile.

4-N-substituted-2-N-substituted-5-fluorobenzenecarbonitrile was reduced with DIBAL-H according to procedure described in Scheme 10 to give 4-N-substituted-2-N-substituted-5-fluorobenzaldehyde.

### Preparation of amino {3-[5-(3-chlorophenyl)(2-furyl)](2-pyrazolinyl)}methane-1-thione

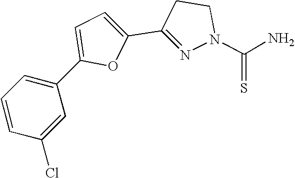

To a solution of 5-(3-chlorophenyl)furan-2-carbaldehyde (1.0 equiv.) in THF at 0° C. was added MeMgBr in ether (3.0 equiv.) and stirred for 45 min. The reaction was quenched with water, diluted with ether and filtered through Celite. The organic layer was separated and washed with brine, dried over MgSO₄, and concentrated to give the 1-[5-(3-chlorophenyl)-2-furyl]ethan-1-ol.

To a solution of secondary alcohol(1.0 equiv.) in CH₂Cl₂ was added MnO₂ (10 equiv.). The reaction was stirred overnight, filtered through Celite, and concentrated to give 1-[5-(3-chlorophenyl)-2-furyl]ethan-1-one.

To a mixture of ketone (1.0 equiv.), paraformaldehyde (2.0 equiv.), and dimethylamine hydrochloride (2.0 equiv) and molecular sieves in ethanol was added concentrated hydrochloric acid (cat.). The reaction was refluxed overnight under nitrogen and the concentrated. A few drops of HCl was added, and the mixture was worked up with DCM and water. The organic layer was discarded. The aqueous layer was adjusted to basic and extracted with DCM (3×). The organic layer was washed with brine, dried over MgSO₄, and concentrated to yield 3-(dimethylamino)-1-[5-(3-chlorophenyl)(2-furyl)]propan-1-one.

Thiosemicarbazide (1.0 equiv.) was dissolved in MeOH upon heating under nitrogen. Aqueous sodium hydroxide (6 M, 9.0 equiv.) was added to the reaction. A methanol solution of 3-(dimethylamino)-1-[5-(3-chlorophenyl)(2-furyl)]propan-1-one (1.0 equiv) was then added dropwise to the reaction mixture. The solvent was removed and the residue was dissolved in DCM and washed with water, brine, dried over MgSO₄, and concentrated. The final compound was purified by preparative-HPLC to give amino {3-[5-(3-chlorophenyl)(2-furyl)](2-pyrazolinyl)}methane-1-thione; LC/MS m/z 306.2 (MH+); Rt=3.06 minutes.

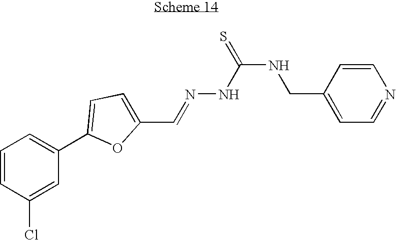

To a solution of 4-pyridylmethylamine (1.0 equiv.) and triethylamine (2.0 equiv.) in CHCl₃ was added CS₂ (1.0 equiv.)) and stirred overnight. The reaction was cooled to 0° C. and ethyl chloroformate (1.0 equiv.) was added dropwise. The reaction was stirred for 15 min at 0° C. and then stirred at room temperature for 2 hrs followed by addition of (tert-butyl)oxycarbohydrazide (1.2 equiv.). After stirring for an addition hour the mixture was washed with aqueous citric acid (5%), saturated NaHCO₃, brine, dried over MgSO₄, and concentrated. The desired Boc protected thiosemicarbazide was purified using column chromatography.

To a solution of Boc protected thiosemicarbazide (1.0 equiv.) dissolved in DCM was added HCl in dioxane (2M, 8.3 equiv.) and stirred for 15 min. MeOH is then added to dissolve the precipitate, followed by addition of the furfural, and small amount of acetic acid (0.5 mL). The mixture is stirred overnight and the solvents are removed to give a residue purified by preparative-HPLC to give the thiosemicarbazone.

### Synthesis of 4-[4-(4-methylpiperazin-1-yl)phenoxymethyl]benzaldehyde

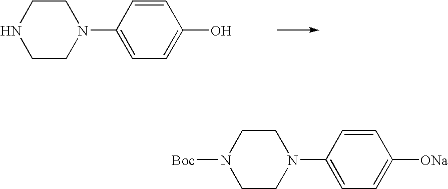

To a solution of 4-piperazin-1-yl phenol (1 equivalent) in CHCl₃, cooled to 0° C., was added di-t-butyl dicarbonate (1 equivalent) in CHCl₃ drop-wise. The solution was stirred at 0° C. for 1 hour before removing from the cold bath and stirring at ambient temperatures for 18 hours. The organic solution was washed aqueous NaHCO₃ and brine dried over MgSO₄ and concentrated the crude material was used without purification.

A solution of the resulting 4-(1-BOC-piperazin-4-yl)phenol (1 equivalent) in dry CH₃CN was slowly added drop-wise to a slurry of NaH (1 equivalent) in dry CH₃CN at room temperature under N₂. The slurry was stirred at room temperature for 2 hours before the solids were filtered and washed with Et₂O.

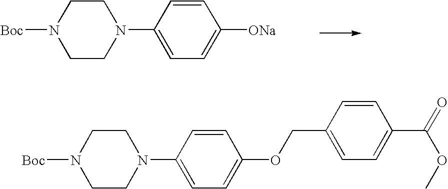

Sodium 4-(1-BOC-piperazin-4-yl)phenoxide (1 equivalent) and methyl 4-bromomethylbenzoate (1 equivalent) were combined in dry acetone and heated to reflux at 60° C. for 18 hours. The slurry was filtered and the filtrate was then concentrated to provide the crude methyl 4-[4-(1-BOC-piperazin-4-yl)phenoxymethyl]benzoate, that was used without purification.

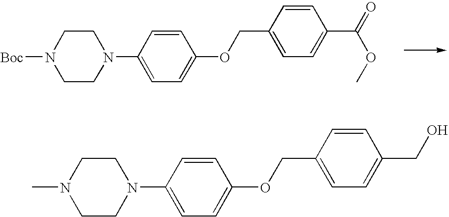

To a slurry of LiAlH₄ (4 equivalents) in dry THF, cooled to 0° C. under N₂, was slowly added drop-wise a solution of methyl 4-[4-(1-BOC-piperazin-4-yl)phenoxymethyl]benzoate (1 equivalent) in dry THF. Once the addition was complete, the slurry was heated to reflux at 80° C. for 1 hour. The slurry was subsequently cooled to 0° C. and treated with water, 10% aq. NaOH and with water again. The resulting solids were filtered, and the filtrate was diluted with chloroform, washed with brine, dried over MgSO₄ and concentrated, providing the crude 4-[4-(4-methylpiperazin-1-yl)phenoxymethyl]benzyl alcohol that was used without purification.

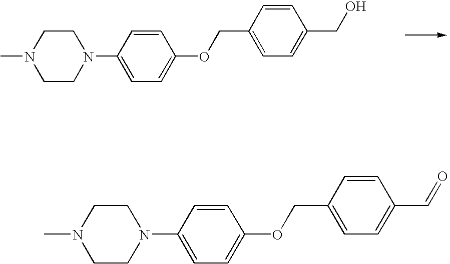

To a solution of DMSO (2.6 equivalents) in dry DCM, cooled to −78° C. under N₂ was added oxalyl chloride (1.1 equivalents) in DCM drop-wise. The solution was stirred at −78° C. for 5 minutes before a solution of 4-[4-(4-methylpiperazin-1-yl)phenoxymethyl]benzyl alcohol (1 equivalent) in DCM was added drop-wise, and allowed to stir at −78° C. for another 30 minutes. Triethylamine (2.5 equivalents) was slowly dripped in before allowing the solution to reach ambient temperatures. The solution was washed with aqueous NaHCO₃ and brine, dried over MgSO₄ and concentrated to provide the crude 4-[4-(4-methylpiperazin-1-yl)phenoxymethyl]benzaldehyde that was converted to thiosemicarbazones according to Scheme 7.
Pyrroles

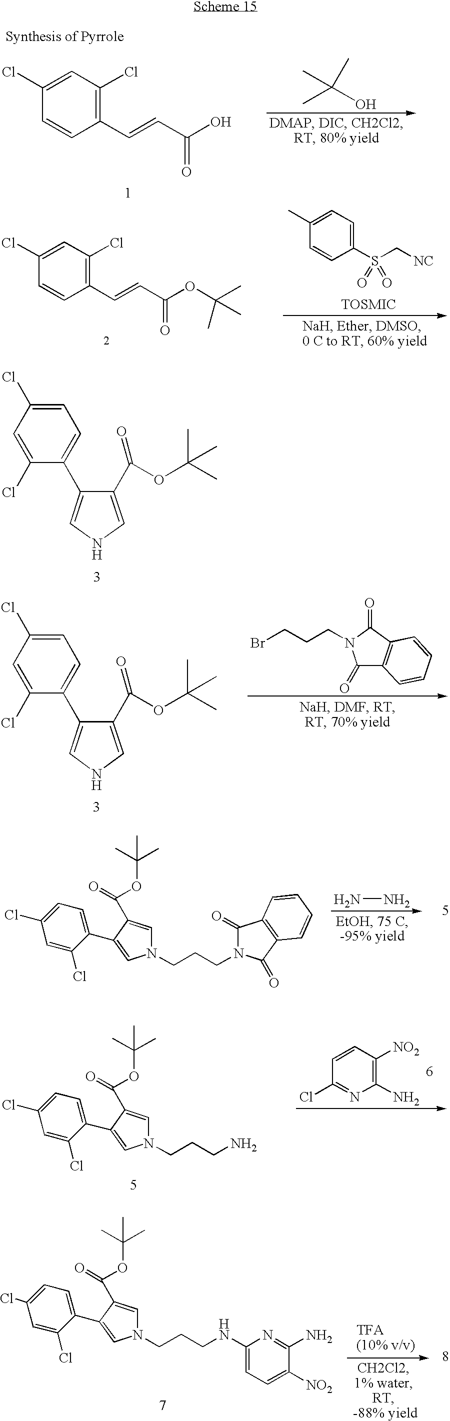

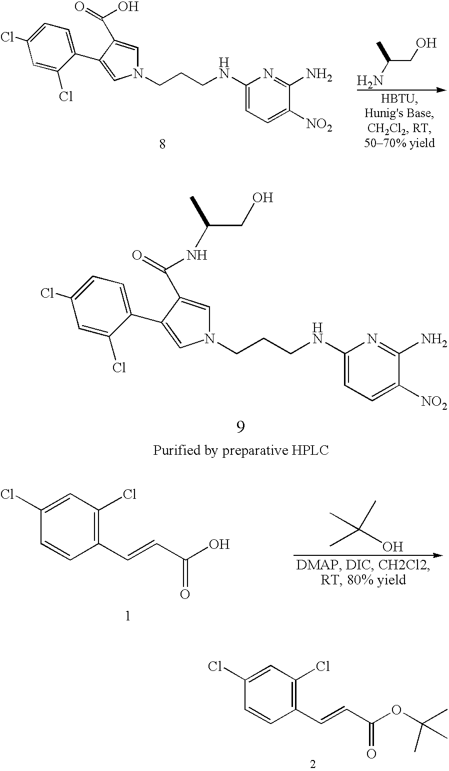

### Preparation of tert-butyl (2E)-3-(2,4-dichlorophenyl)prop-2-enoate (2)

Neat DIC (1.4 eq) was added to a well stirred solution of cinnamate (1 eq), t-butyl alcohol (4 eq), DMAP (1.4 eq) and CH₂Cl₂ under argon at rt. (Note—The cinnamate must be completely in solution that may require gentle warming. Allow the solution to cool to room temperature before adding the DIC. To avoid an exotherm on larger scales, it may be prudent to dilute the DIC with CH₂Cl₂ before the addition and have an ice bath ready.) After stirring for 8 hours, the reaction develops a white precipitate. The reaction may be monitored by TLC eluting with 25% EtOAc/Hexane (R_{f} of product was 0.9). The entire reaction was loaded into a separatory funnel (washing with CH₂Cl₂). The organic mixture was washed with citrate, sat. aq. NaHCO₃, water, and brine. The organic layer was dried (Na₂SO₄), filtered, and concentrated to dryness to give the crude product as an oil. The crude oil was mixed with hexane and stirred for 30 min. The precipitate that forms was filtered over celite and the filtrate was evaporated. The hexane mixture was loaded onto a filter plug of silica and eluted with EtOAc/hexane (97:2 v/v). The first eluted UV active fractions are collected and evaporated to give >99% pure 2 (75-80% yields).

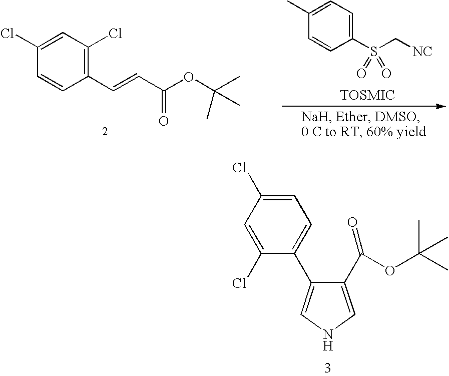

### Preparation of tert-butyl 4-(2,4-dichlorophenyl)pyrrole-3-carboxylate (3)

Dry ether was added to NaH (1.5 eq as the oil dispersion) under argon. After decanting off the ether via syringe, the NaH was suspended again with fresh ether under argon. A solution of TOSMIC (1.1 eq) and 2 (1 eq) dissolved in a mixture of ether and DMSO was added dropwise to the stirred suspension of NaH at 0° C. over 20-30 min. The addition was mildly exothermic and evolved gas. After the addition, the reaction was allowed to warm to ambient rt. The progress of the reaction was followed by TLC (25% EtOAc/Hexane, the UV active product was at R_{f}=0.4) and LCMS until done (˜2-3 h). Upon completion, the reaction was carefully quenched with sat. aq. NH₄Cl (added slowly to avoid strong gas evolution and exotherm) and diluted with ether. The layers were separated and the organic phase was washed with sat. aq. NaHCO₃, water, and brine. The crude dark solid can be purified by recrystallization. Best results were achieved either through recrystallization directly from a mixture of hot EtOAc/hexane (1:3 v/v) or by dissolving the crude product in minimal hot EtOAc followed by addition of hexane (˜2 volumes of hexane based on the volume of EtOAc). The hot solutions were allowed to cool to room temperature and age over night. The crystals were first filtered and then washed with hexane giving 99% pure product in 60-70% yield.

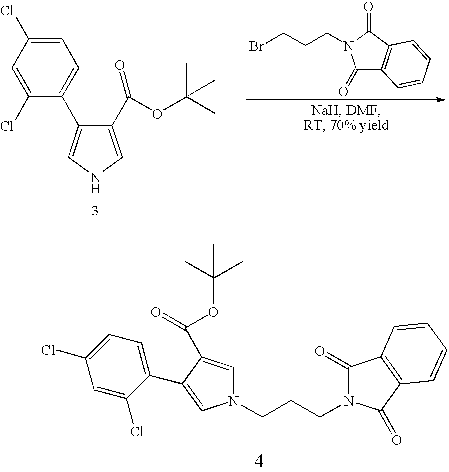

### Preparation of tert-butyl 4-(2,4-dichlorophenyl)-1-[3-(1,3-dioxobenzo[c]azolin-2-yl)propyl]pyrrole-3-carboxylate (4)

Solid NaH (1.5 eq as the oil dispersion) was added in small portions to a solution of pyrrole 3 (1 eq) and 3-bromopropyl phthalimide (1.2 eq) dissolved in DMF stirred at room temperature and flushed with argon. NOTE—Some gas evolves, but the temperature does not seem to rise above 40-50° C. The reaction was stirred for 1.5 h at room temperature under argon. The reaction was followed by TLC (CH₂Cl₂/acetonitrile (95:5 v/v), the UV active product was at R_{f}=0.5) and LCMS. Upon completion, the reaction was quenched with sat. aq. NH₄Cl (add slowly to avoid strong gas evolution and exotherm). Sat. aq. NaHCO₃ was then added to avoid an emulsion, and the basic organic mixture was extracted with ether. The combined ether layers were washed with sat. aq. NaHCO₃, water, brine, dried Na₂SO₄, filtered, and concentrated to dryness to give the crude product. The crude product was purified by eluting through silica with EtOAc/Hexane (1:4 v/v). The purified product contained some residual 3-bromopropyl phthalimide, that did not interfere with subsequent synthetic steps. The material was taken on and used without further purification. Assume a quantitative yield.

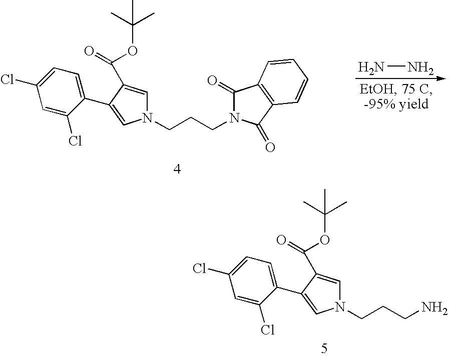

### Preparation of tert-butyl 1-(3-aminopropyl)-4-(2,4-dichlorophenyl)pyrrole-3-carboxylate (5)

The Pthalimido Pyrrole 4 (1 eq) was dissolved in ethanol and hydrazine (3 eq) at room temperature under nitrogen. Upon heating to reflux, the reaction generated a white precipitate. Stir at reflux until complete (˜2 h) by TLC (CH₂Cl₂/acetonitrile (95:5 v/v), the UV active product was at R_{f}=0.2) and LCMS. Upon reaching completion, the reaction was allowed to cool to room temperature and the precipitate was vacuum-filtered off using a medium to fine cintered-glass filter. The filtrate was concentrated under reduced pressure to a gummy solid. The crude material was taken up in ethanol/EtOAc (1:1 v/v), stirred and the precipitate was filtered off in the same fashion as before. The filtrate was concentrated under reduced pressure and than dried in vacuo for 10-15 min. This process of adding ethanol/EtOAc, filtering and concentrating was done one more time or as needed to remove the majority of the white precipitate and residual hydrazine. The product was then dried in vacuo overnight. The material was used without further purification. Once dried, the reaction yielded the product as a glass (˜87% yield over 2 steps).

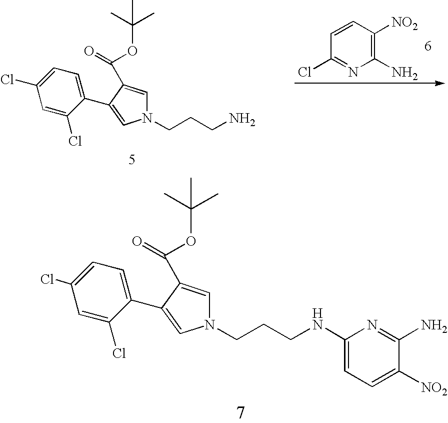

### Preparation of tert-butyl 1-{3-[(6-amino-5-nitro(2-pyridyl))amino]propyl}-4-(2,4-dichlorophenyl)pyrrole-3-carboxylate (7)

To the premixed dry reagents, pyrrole 5 (1 eq) and powdered 6-chloro-3-nitro-2-pyridylamine (6) (1.1 eq), was added the DMA followed by Hünig's base (2 eq) sequentially with stirring at rt. The reaction was then heated to 80° C. overnight. The reaction was followed by TLC (EtOAc/hexane (1:1 v/v), the UV active yellow product was at R_{f}=0.25), HPLC and LCMS. Upon completion as judged by HPLC, the reaction was allowed to cool to 70° C. Ethylene diamine (anhydrous) was then added to the reaction to destroy any remaining unreacted chloropyridine 6. After 15 min stirring at 70° C., the reaction was cooled and quenched with the addition of sat. aq. NaHCO₃. The aqueous mixture was extracted with EtOAc, and the combined organic layers were washed with sat. aq. NaHCO₃, water, brine, dried, filtered, and concentrated to dryness to give the crude product as a brown-yellow solid. The crude product was purified by flash chromatography eluted with EtOAc/hexane (4:6 v/v). The purified SnAr adduct 7 was isolated in 58% yield as a yellow solid.

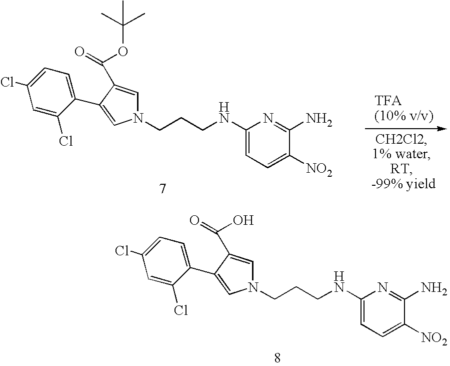

### Preparation of 1-{3-[(6-amino-5-nitro(2-pyridyl))amino]propyl}-4-(2,4-dichlorophenyl)pyrrole-3-carboxylic acid (8)

In a vial, TFA (catalytic amount) was added to a stirred mixture of tert-butyl ester pyrrole 7 (1 eq), water (0.1%), and CH₂Cl₂ at rt. The vial stirred at room temperature until done (˜12 h. The reaction was then concentrated under reduced pressure at room temperature and dried in vacuo. The crude residue was dissolved again in CH₂Cl₂ and concentrated under reduced pressure at rt. The material was used in the final coupling step without further purification as the TFA salt.

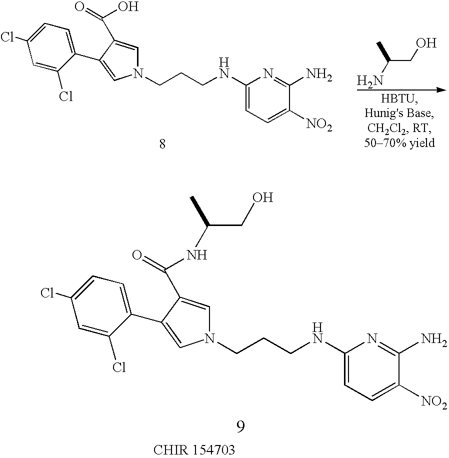

### Preparation of N-((1S)-2-hydroxy-isopropyl)(1-{3-1(6-amino-5-nitro(2-pyridyl))amino]propyl}-4-(2,4-dichlorophenyl)pyrrol-3-yl)carboxamide (9,)

(2S)-(+)-2-Aminopropan-1-ol (1.5 eq) was added to a stirred mixture of acid (8) (1 eq), HBTU (1.5 eq), Hünig's base (2 eq) and DMF (premixed sequentially in this order in a vial) at room temperature under argon. The reaction was stirred for 3-4 h until complete as shown by LCMS and HPLC. The reaction mixture was subsequently diluted with EtOAc, washed with NaHCO₃, and concentrated to afford a powder in a 70% yield.

Nomenclature for the Example compounds was provided using ACD Name version 5.07 software (Nov. 14, 2001) available from Advanced Chemistry Development, Inc. Some of the compounds and starting materials were named using standard IUPAC nomenclature.

The compounds of Table 34 were synthesized following the synthetic methodology described above in the Examples and Schemes, and screened following methods 1 and 2 below. The precursors are readily recognizable by one skilled in the art and are commercially available from Aldrich (Milwaukee, Wis.) or Acros Organics (Pittsburgh, Pa.), among others.

### Screening Methods for SMIP/SMIS Compounds

Method 1

Candidate small molecule immuno-potentiators can be identified in vitro. Compounds are screened in vitro for their ability to activate immune cells. One marker of such activation is the induction of cytokine production, for example TNF-α production. Apoptosis inducing small molecules may be identified having this activity. These small molecule immuno-potentiators have potential utility as adjuvants and immuno-therapeutics.

In an assay procedure (High Throughput Screening (HTS)) for small molecule immune potentiators (SMIPs), human peripheral blood mononuclear cells (PBMC), 500,000 per mL in RPMI 1640 medium with 10% FCS, were distributed in 96 well plates (100,000 per well) already containing 5 μM of compound in DMSO. The PBMCs were incubated for 18 h at 37° C. in 5% CO₂. Their ability to produce cytokines in response to the small molecule compounds is determined using a modified sandwich ELISA.

Briefly supernatants from the PBMC cultures were assayed for secreted TNF using a primary plate bound antibody for capture followed by a secondary biotinylated anti-TNF antibody forming a sandwich. The biotinylated second antibody was then detected using streptavidin-Europium and the amount of bound europium was determined by time resolved fluorescence. SMIP compounds were confirmed by their TNF inducing activity that was measured in the assay as increased Europim counts over cells incubated in RPMI medium alone. “Hits” were selected based on their TNF-inducing activity relative to an optimal dose of lipopolysaccaride LPS (1 μg/ml), a strong TNF inducer. The robustness of the assay and low backgrounds allowed for the routine selection of hits with ˜10% of LPS activity that was normally between 5-10× background (cells alone). Selected hits are then subjected to confirmation for their ability to induce cytokines from multiple donors at decreasing concentrations. Those compounds with consistent activity at or below 5 μM are considered confirmed for the purposes of this assay. The assay is readily modified for screening for compounds effective at higher or lower concentrations.

Method 2

Each of the compounds in the above Table 34 elicited TNF-α production in human peripheral blood mononuclear cells. Many of the compounds showed activity at less than 20 μM with respect to production of TNF-α. Many of these compounds showed activity at less than 5 μM with respect to production of TNF-α. Many of these compounds showed activity in the production of TNF-α at less than 1.5 μM.

For this reason, each of the R groups of any of the compounds listed in Table 34 are preferred. Additionally, because of the excellent activity of each of the compounds, each of these compounds is individually preferred and is preferred as a member of a group that includes any or all of the other compounds and each compound is preferred in methods of modulating immunopotentiation and in methods of treating biological conditions associated therewith, for example to be used as a vaccine adjuvant. Each of the compounds is also preferred for use in preparation of medicaments for vaccines, immunopotentiation, reducing tumor growth and in treating biological conditions mediated therefrom.

In additon to the procedure described above, methods of measuring other cytokines (e.g. IL1-beta, IL-12, IL-6, IFN-gamma, IL-10 etc.) are well known in the art and can be used to find active SMIP compounds of the present invention.

Compounds may be useful that cause production of TNF-α at higher concentrations, such as 100 μM, 200 μM or 300 μM in the assays described herein. For example Loxoribine causes useful production of TNF-α at 300 μM (see Pope et al Immunostimulatory Compound 7-Allyl-8-Oxoguanosine (Loxoribine) Induces a Distinct Subset of Murine Cytokines Cellular Immunology 162: 333-339 (1995)).

The subject invention also includes isotopically-labeled antiviral compounds, that are structurally identical to those disclosed above, but for the fact that one or more atoms are replaced by an atom having an atomic mass or mass number different from the atomic mass or mass number usually found in nature. Examples of isotopes that can be incorporated into antiviral compounds of the invention include isotopes of hydrogen, carbon, nitrogen, oxygen, phosphorous, sulfur, fluorine and chlorine, such as ²H, ³H, ¹³C, ¹⁴C, ¹⁵N, ¹⁸O, ¹⁷O, ³¹P, ³²P, ³⁵S, ¹⁸F and ³⁶Cl, respectively. Antiviral compounds of the present invention, derivatives thereof, and pharmaceutically acceptable salts of said compounds and of said derivatives that contain the aforementioned isotopes and/or other isotopes of other atoms are within the scope of this invention. Certain isotopically-labeled antiviral compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, may afford certain therapeutic advantages resulting from greater metabolic stability, for example increased in vivo half-life or reduced dosage requirements and, hence, may be preferred in some circumstances. Isotopically labeled antiviral compounds of this invention and derivatives thereof can generally be prepared by carrying out known or referenced procedures and by substituting a readily available isotopically labeled reagent for a non-isotopically labeled reagent.

In accordance with the present invention, methods are provided for the administration of an effective amount of a SMIP compound to act as an adjuvant. Also provided are immunogenic compositions comprising a SMIP compound, an antigen, and optionally other adjuvants.

As adjuvants, the SMIP compounds are combined with antigens and delivery systems to form a final immunogenic composition or vaccine product.

As immunotherapeutics, the SMIP compounds are used alone or in combination with other therapies for treatment of SARS.

Those of ordinary skill in the art will recognize that physiologically active antiviral compounds, SMIPs or SMISs that have accessible hydroxy groups are frequently administered in the form of pharmaceutically acceptable esters. The antiviral compounds of this invention can be effectively administered as an ester, formed on the hydroxy groups, just as one skilled in pharmaceutical chemistry would expect. It is possible, as has long been known in pharmaceutical chemistry, to adjust the rate or duration of action of the antiviral compound by appropriate choices of ester groups.

Other compounds that can be used in combination with the therapeutic agents described herein include, pentoxifylline (PTX), methylprednisolone, trimetrexate (Neutrexin), Zadaxin (thymosin alpha 1), optionally substituted 5-aminomethinimino-3-methyl-4-isoxazolecarboxylic acid phenylamides, cyclosporine A (CsA), 6-oxo-1,4,5-thiadiazin[2,3-b]quinazoline, 3-amino-2(1H)-thioxo-4(3H)-quinazolinone, gangciclovir, glycyrrhizin, tetracyclines, aminoglycosides, quinolones, bicyclam (1,4-Bis(1,4,8,11-tetraazacyclotetradec-1-ylmethyl)benzene octahydrochloride dihydrate), rapamycin, wortmannin, enalapril, roquinimex/linomide, inactivin, DNCB, AG7088, 9-aminocamptothecin (CPT-11), loxorobine, bropirimine, Ononase ® (ranpimase), statins, such as: lovastatin—Mevacor®, pravastatin—Pravachol®, simvastatin—Zocor®, fluvastatin—Lescol®, atorvastatin—Lipitor® and rosuvastatin—Crestor®.

As used herein, the term “effective amount” means an amount of antiviral compound of the compositions, kits and methods of the present invention that is capable of treating the symptoms of the described conditions. The specific dose of a compound administered according to this invention will, of course, be determined by the particular circumstances surrounding the case including, for example, the compound administered, the route of administration, the state of being of the patient, and the severity of the condition being treated.

The dose of an antiviral compound of this invention to be administered to a subject is rather widely variable and subject to the judgment of the attending physician. It should be noted that it may be necessary to adjust the dose of a compound when it is administered in the form of a salt, such as a laureate, the salt forming moiety of which has an appreciable molecular weight.

The following dosage amounts and other dosage amounts set forth elsewhere in this description are for an average human subject having a weight of about 65 kg to about 70 kg. The skilled practitioner will readily be able to determine the dosage amount required for a subject whose weight falls outside the 65 kg to 70 kg range, based upon the medical history of the subject and the presence of diseases, e.g., diabetes, in the subject. Calculation of the dosage amount for other forms of the free base form such as salts or hydrates is easily accomplished by performing a simple ratio relative to the molecular weights of the species involved.

In general, the pharmaceutical compositions will include at least one antiviral compound in combination with a pharmaceutically acceptable vehicle, such as saline, buffered saline, 5% dextrose in water, borate-buffered saline containing trace metals or the like. Formulations may further include one or more excipients, preservatives, solubilizers, buffering agents, lubricants, fillers, stabilizers, etc. Methods of formulation are well known in the art and are disclosed, for example, in “Remington's Pharmaceutical Sciences,” Mack Pub. Co., New Jersey (1991) or “Remington: The Science and Practice of Pharmacy,” 20^{th} ed., Lippincott Williams & Wilkins, Baltimore, Md. (2000), incorporated herein by reference.

Pharmaceutical compositions for use within the present invention can be in the form of sterile, non-pyrogenic liquid solutions or suspensions, coated capsules, suppositories, lyophilized powders, transdermal patches or other forms known in the art.

Many of the active ingredient antiviral compounds are known to be absorbed from the alimentary tract, and so it is usually preferred to administer a compound orally for reasons of convenience. However, the compounds may equally effectively be administered intravenously, subcutaneously, percutaneously, or as suppositories for absorption by the rectum or vagina, if desired in a given instance. All of the usual types of compositions may be used, including tablets, chewable tablets, capsules, solutions, parenteral solutions, troches, suppositories and suspensions. Compositions are formulated to contain a daily dose, or a convenient fraction of daily dose, in a dosage unit, that may be a single tablet or capsule or convenient volume of a liquid.

Capsules are prepared by mixing the compound or compounds with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound or compounds. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant is generally necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances that swell when wetted to break up the tablet and release the compound or compounds. They include starches, clays, celluloses, algins and gums, more particularly, corn and potato starches, methylcellulose, agar, bentonite, wood cellulose, powdered natural sponge, cation-exchange resins, alginic acid, guar gum, citrus pulp and carboxymethylcellulose, for example, may be used as well as sodium lauryl sulfate.

Tablets are often coated with sugar as a flavor and sealant, or with film-forming protecting agents to modify the dissolution properties of the tablet. The compounds may also be formulated as chewable tablets, by using relatively large amounts of pleasant-tasting substances such as mannitol in the formulation, as is now well-established in the art.

When it is desired to administer a compound as a suppository, the typical bases may be used. Cocoa butter is a traditional suppository base, that may be modified by addition of waxes to raise its melting point slightly. Water-miscible suppository bases comprising, particularly, polyethylene glycols of various molecular weights are in wide use.

The effect of the compounds may be delayed or prolonged by proper formulation. For example, a slowly soluble pellet of the compound may be prepared and incorporated in a tablet or capsule. The technique may be improved by making pellets of several different dissolution rates and filling capsules with a mixture of the pellets. Tablets or capsules may be coated with a film that resists dissolution for a predictable period of time. Even the parenteral preparations may be made long-acting by dissolving or suspending the compound or compounds in oily or emulsified vehicles that allow dispersion slowly in the serum.

The combinations of this invention may be administered in a controlled release formulation such as a slow release or a fast release formulation. Such controlled release formulations of the combination of this invention may be prepared using methods well known to those skilled in the art. The method of administration will be determined by the attendant physician or other person skilled in the art after an evaluation of the subject's condition and requirements.

The term “prodrug” means compounds that are transformed in vivo to yield an antiviral compound of the present invention. The transformation may occur by various mechanisms, such as through hydrolysis in blood. A good discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987. The term, “prodrug” also encompasses mutual prodrugs in which one or more antiviral compounds are combined in a single molecule that may then undergo transformation to yield the individual antiviral compounds of the present invention.

For example, if an antiviral compound of the present invention contains a carboxylic acid functional group, a prodrug can comprise an ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or morpholino(C₂-C₃)alkyl.

Similarly, if an antiviral compound of the present invention comprises an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If an antiviral compound of the present invention comprises an amine functional group, a prodrug can be formed by the replacement of a hydrogen atom in the amine group with a group such as R^{X}-carbonyl, R^{X}O-carbonyl, NR^{X}R^{X}′-carbonyl where R^{X} and R^{X}′ are each independently ((C₁-C₁₀)alkyl, (C₃-C₇)cycloalkyl, benzyl, or R^{X}-carbonyl is a natural α-aminoacyl or natural α-aminoacyl-natural α-aminoacyl, —C(OH)C(O)OY^{X} wherein (Y^{X} is H, (C₁-C₆)alkyl or benzyl), —C(OY^{X}O)Y^{X1} wherein Y^{X0} is (C₁-C₄)alkyl and Y^{X1} is ((C₁-C₆)alkyl, carboxy(C₁-C₆)alkyl, amino(C₁-C₄)alkyl or mono-N- or di-N,N—(C₁-C₆)alkylaminoalkyl, —C(Y^{X2})Y^{X3} wherein Y^{X2} is H or methyl and Y^{X3} is mono-N- or di-N,N—(C₁-C₆)alkylamino, morpholino, piperidin-1-yl or pyrrolidin-1-yl.

The compositions for use in accordance with the present invention may be formulated in a conventional manner using one or more physiologically acceptable carriers or excipients. Antiviral, SMIP, SMIS, or other immunomodulating compounds are prepared or obtained as described herein and in the U.S. Patents and published international patent applications listed in Table 1, Table 2, Table 34 and Table 35. The antiviral compounds can be formulated in pharmaceutically acceptable compositions suitable for delivery to the lungs. Particular formulations include dry powders, liquid solutions or suspensions suitable for nebulization and propellant formulations suitable for use in metered dose inhalers. The preparation of such formulations is well know to those skilled in the art, and is described in U.S. Pat. Nos. 5,814,607 and 5,654,007 and in the U.S. Patents and published international patent applications listed in Table 3 the disclosures of which are incorporated herein by reference.

Dry powder formulations will comprise an antiviral compound in a dry, optionally lyophilized form with a particle size within a preferred range for deposition within the lung. Typically the particle size for deposition in the lung will range between 1 and 5 μm. When systemic delivery of the antiviral compound via absorption from the lung into the bloodstream is desired the antiviral compound formulation particle size is generally between 0.1 and 2 μm in size. The preferred size range of particles can be produced using methods such as jet-milling, spray drying and solvent precipitation, for example. Dry powder devices typically require a powder mass in the range from about 1 mg to 100 mg to produce an aerosolized dose. Thus, the antiviral compound will typically be combined with a pharmaceutically acceptable dry bulking powder. Preferred dry bulking powders include sucrose, lactose, trehalose, human serum albumin (HSA), phospholipids and glycine as well as those disclosed in the documents listed in Table 3. Dry powders can be administered to the subject in conventional dry powder inhalers. For liquid formulations the antiviral compound can be dissolved in any recognized physiologically acceptable carrier for use in delivery of aerosolized formulations. Such carriers include buffered and unbuffered aqueous solutions for water soluble compounds, and physiological solutions including saline solution (preferably between 0.2 and 2 N NaCl). For antiviral compounds with limited solubility, other liquid vehicles such as ethanol, propylene glycol and ethanol-propylene combinations may be used. The antiviral compounds may also be administered as solids in suspension.

For administration by inhalation, the compositions for use according to the present invention are conveniently delivered in the form of an aerosol spray administered via pressurized packs or a nebulizer, with the use of a propellant, e.g., air, dichlorordifluoromethane, dichloroterafluoroethane or other suitable gas. Preferably, for incorporation into the aerosol propellant, the antiviral compound formulations of the present invention will be processed into respirable particles as described above for the dry powder formulations. The particles are then suspended in the propellant, optionally being coated with a surfactant to enhance their disbursement. In the use of a pressurized aerosol the dosage unit may be determined by providing a valve to deliver a metered amount.

Commercially available jet nebulizers are available and may be used to deliver aerosolized antiviral compound to a subject. Such jet nebulizers include, but are not limited to, those supplied by AeroTech 11 (CIS-US, Bedford, Mass.). In addition, for delivery of aerosolized antiviral compound to the lungs of a subject an oxygen source can be attached to the nebulizer providing a flow rate of, for example, 10 L/min. In general, inhalation is performed over a 5-40 minute time interval through a mouthpiece during spontaneous respiration. The present invention provides for novel compositions comprising a suitable carrier and aerosolized antiviral compound in doses sufficient to reduce or ameliorate viral load and SARS symptoms in subjects having SARS. Such doses can be lower than corresponding systemic doses that may be used to those generally used to reduce or ameliorate viral load and SARS symptoms in subjects having SARS.

The antiviral, SMIP, SMIS, and immunomodulating compositions of the present invention may be administered with a steroidal anti-inflammatory drug for the treatment of SARS and SARS symptoms. Examples of steroidal anti-inflammatory drugs of the invention include hydrocortisone, prednisolone, dexamethasone, triamcinolone acetonide, fluocinolone acetonide, fludrocortisone acetate, betamethasone, etc.

The antiviral compound composition of the invention is nebulized predominantly into particle sizes allowing a delivery of the drug into the terminal and respiratory bronchioles. For efficacious delivery of antiviral compound to the lung endobronchial space of airways in an aerosol, the formation of aerosol particles having mass medium average diameter predominantly between 1 to 5 μm is necessary. The formulation must additionally provide conditions that would not adversely affect the functionality of the airways. Consequently, the formulation must contain enough of the drug formulated under the conditions that allow its efficacious delivery while avoiding undesirable reaction.

For liquid solutions and suspensions, the choice of the nebulizer is made from among commercially available nebulizers. The jet nebulizers known as Sidestream O, obtained from Medicaid and Pari LCS, LC Plus, and eFlow obtained from Pari Respiratory Equipment, Richmond, Va., are examples of typical nebulizers suitable for the practice of the invention. Ultrasonic nebulizers that produce appropriate particle sizes of about 1 to 5 μm such as Aerosonic by DeVilbiss and UltraAire by Omron are also suitable.

Advantageously, the present invention also provides for a kit for use by a consumer for the treatment and/or prevention of SARS. Such a kit comprises: (a) a pharmaceutical composition comprising a therapeutically effective amount of at least one compound from among those described herein or listed in Table 34 and Table 35 or described in the U.S. Patents and published international patent applications listed in Table 1, Table 2, and Table 35 and a pharmaceutically acceptable carrier, vehicle or diluent; (b) a container for holding the pharmaceutical composition; and, optionally, (c) instructions describing a method of using the pharmaceutical compositions for the treatment and or the prevention of SARS. The kit may optionally contain a plurality of antiviral compounds for the treatment of SARS wherein the anti viral compounds are selected from 3C-like protease inhibitors and papain-like protease inhibitors. In a further embodiment, the kit contains an antiviral compound which is an RNA-dependent RNA polymerase inhibitor. When the kit comprises more than one antiviral compound, the antiviral compounds contained in the kit may be optionally combined in the same pharmaceutical composition.

A “kit” as used in the instant application includes a container for containing the separate compositions such as a divided bottle or a divided foil packet. The container can be in any conventional shape or form as known in the art that is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil that is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It maybe desirable to provide a written memory aid, where the written memory aid is of the type containing information and/or instructions for the physician, pharmacist or subject, e.g., in the form of numbers next to the tablets or capsules whereby the numbers correspond with the days of the regimen which the tablets or capsules so specified should be ingested or a card that contains the same type of information. Another example of such a memory aid is a calendar printed on the card e.g., as follows “First Week, Monday, Tuesday,” . . . etc . . . . “Second Week, Monday, Tuesday, . . . ” etc. Other variations of memory aids will be readily apparent. A “daily dose” can be a single tablet or capsule or several tablets or capsules to be taken on a given day. Also a daily dose of one or more component(s) of the kit can consist of one tablet or capsule while a daily dose of another one or more component(s) of the kit can consist of several tablets or capsules.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### EXAMPLES

### Example 1

### Example of a SARS Virus Isolate

A SARS virus was isolated from clinical specimens of a patient in Frankfurt, Germany (FRA). The isolate was grown in Vero cells. RNA of the SARS virus was extracted and amplified by RT-PCR. Nucleotide sequence of the viral genome was determined by direct sequencing of the PCR product. Computer analysis was used to predict the features of the genome, to compare it to previously known coronaviruses and to the sequence of different SARS virus isolates.

More specifically, isolation and sequence was performed as follows. After the third passage of the SARS virus in Vero cells, viral particles were purified by ultra centrifugation from 3×10⁷ cells supernatant. Viral RNA was extracted by Triazol method (Gibco-BRL). Viral RNA (200 ng) was transcribed into cDNA with avian RNaseH-thermostable reverse transcriptase following the instructions of the manufacturer (ThermoScript RT System, Invitrogen). Briefly, either 50 pmoles of oligo (dT)₂₀ (SEQ ID NO: 7389) or 25 ng of random hexamers were used to prime the RT reaction in a 20 μl final volume. Amplification and sequencing of the SARS genome were accomplished by direct sequencing of PCR products obtained with: i) specific primers from conserved regions of homology found through multiple alignment among known coronaviruses; ii) oligonucleotides designed around short sequences of SARS isolates available on the Web through WHO network laboratories; iii) degenerate primers to amplify the cDNA mixture with multiple overlapping fragments as end products. Gap closure was realized by long distance PCR with high fidelity Taq (Expand High Fidelity system, Roche) using primers designed on selected fragments. Sequence was collected by primer walking using a BigDye terminator chemistry (Applied Biosystems) and an automated DNA sequencer (3700 capillary model, Applied Biosystems). After obtaining a first pass of the entire genome, a set of both forward and reverse primers were used to amplify and sequence de novo the genome using as a template DNA segments of 2 kb on average. Readings from overlapping fragments were automatically assembled by AutoAssembler (Applied Biosystems) and the 29,740 bp contiguous edited manually.

Computer analysis of the sequence was performed as follows. The GCG Wisconsin Package suite (version 10.0) was used for computer analysis of gene and protein sequences. The PSORT program (http://psort.nibb.ac.jp/) was used for localization predictions. For secondary structure analysis, the PHD software available on the Web at http://cubic.bioc.columbia.edu/predictprotein/was applied. The PSI-BLAST algorithm was used for homology searches (http://www.ncbi.nlm.nih.gov/blast) using the non-redundant protein database. ClustalW was applied to obtain multiple sequence alignments of gene and protein sequences. The LeamCoil-VMF program was used to predict coiled-coil regions in the spike proteins (http://learncoil-vmf.lcs.mit.edu/cgi-bin/vmf). Leucine zippers were predicted with the program 2ZIP, available at http://2Zip.molgen.mpg.de.

Phylogenetic analysis was performed using the neighbor-joining algorithm as implemented in the program NEIGHBOR within the Phylogeny Inference Package (Phylip) (Felsenstein J 1993, program distributed by the author). Bootstrap analysis was always performed with 100 replicates using the program Seqboot. Trees were handled and displayed using TreeView. The program HMMER was used to generate sequence profiles from multiple sequence alignments of the S1 domains of spike proteins. Subsequently, the HMMPFAM program was used to compare the S1 domain of SARS spike to the profiles.

The genome of this SARS virus isolate is 29,740 bases long and the overall structure of the genome is similar to that of the three known groups of coronaviruses. Starting from the 5′ end a leader sequence, an untranslated region (UTR) and two overlapping open reading frames coding for one polyprotein containing the enzymes necessary for replication can be identified. They are followed by a region coding for the spike (S), envelope (E), matrix (M), nucleocapsid (N) structural proteins and eight additional ORFs specific for the SARS virus. At the 3′-end of the genome a UTR with a poly(A) is located. The overall homology to coronaviruses groups 1, 2 and 3 is low and therefore the SARS virus belongs to a new group (group 4) of coronavirus. More detailed analysis of the spike protein amino acid sequence shows that the SARS virus isolate is more closely related to coronavirus group 2.

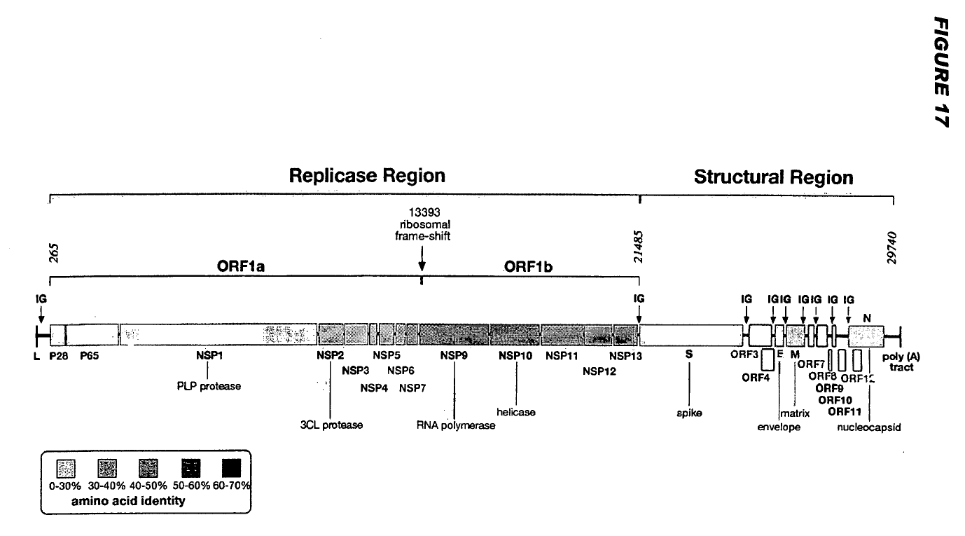

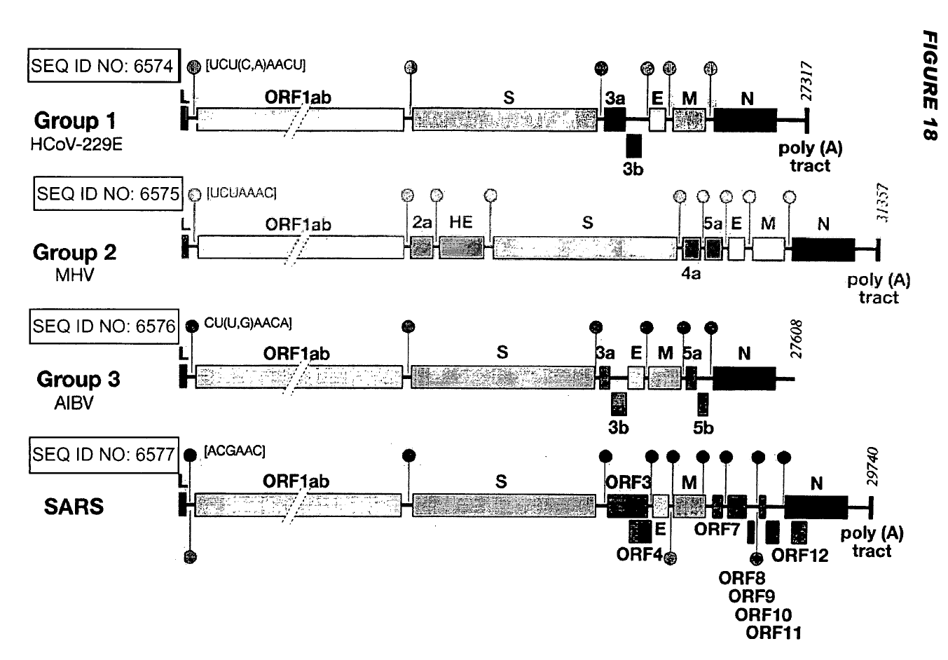

The complete genome sequence of the SARS virus isolate is 29,740 bp in length. The sequence is available on Genbank and has a GC content of 40.8%, comparable with that of known viruses of the same family. Genome structure is similar to that of other coronaviruses. 14 open reading frames have been predicted. The principal features of the genome and gene products are illustrated reported in FIG. 17 and Table 10. The comparison between the SARS genome and those of group 1, 2 and 3 coronaviruses is reported in FIG. 18.

Nucleotides 1-73 contain a predicted RNA leader sequence followed by an untranslated region (UTR) of 197 nucleotides. The UTR is followed by two overlapping open reading frames (ORF1a, ORF1b), which encompass two-thirds of the genome (nucleotides 265-21485). They encode for a large polyprotein, which is predicted to be processed by viral proteases to generate the replicase complex. The 3′ part of the genome contains the genes coding for the four structural proteins (S, spike protein, E, envelope protein, M, matrix glycoprotein, and N, nucleocapsid protein), and eight predicted ORFs of unknown function (FIG. 17). Finally, at the 3′ end of the genome, we found a second UTR of 340 bases followed by a poly(A) tract. We identified a putative intergenic (IG) sequence also referred to as transcription-associated sequence (TAS), which is a typical feature for coronaviruses. The IG sequence is characterized by 6-18 nucleotides present at the 3′ end of the leader and can be found in front of each gene. The IG sequence plays a key role in RNA transcription and its regulation. The IG sequence of the SARS virus is characterized by the sequence SEQ ID NO: 7293 and is present nine times in the genome (FIG. 17). The sequence of the leader and IG are peculiar for each coronavirus and represent a specific signature for the virus.

The Replicase Region

The replicase gene, ORF1ab (SEQ ID NO: 7232), consists of two overlapping ORFs, ORF1a and ORF1b, which can be translated as a single polyprotein by frame shift of the ribosome in position 13,393, within the polymerase encoding region. See Brierley et al, Embo J 1987: 6(12): 3779-3785. As expected, a stem-loop sequence is present ten base pairs downstream of this site (SEQ ID NO: 7390; 5′-CGGTGTAAGTGCAGCCCGTCTTACACCG-3′). The polyprotein is cleaved co- and/or post-translationally into multiple proteins by its own encoded proteases. Using the cleavage consensus sequence and by analogy with other coronaviruses, we have mapped the possible cleavage sites of the polyprotein and have identified 14 products, which comprise the leader protein p28, the homologue of the MHV p65 protein and other twelve proteins, named from nsp1 to nsp13 (nsp, non structural protein) (FIG. 17 and Table 10). The amino acid sequence analysis suggests the presence of several functional motifs within the putative ORF1ab proteins. In particular, we have mapped two potential proteases (nsp1 and nsp2), one growth factor-like motif (nsp7) within ORF1a, whereas in ORF1b we identified the RNA polymerase (nsp9), and a predicted helicase (nsp10). The other predicted cleavage products (nsp3, nsp4, nsp5, nsp6, nsp11, nsp12 and nsp13) are proteins of unknown function. Many of these proteins are presumably present in the RNA replication complex, which is associated with the membranous structures in the infected cells. In particular, nsp3 and nsp4 contain hydrophobic domains. As shown in FIG. 18, the replicase region of SARS has a similar organization to group 1, 2 and 3 coronaviruses; however, the overall aminoacid conservation is low (Table 11). The most conserved proteins are the polymerase and the helices.

Nsp1 is the papain-like cysteine protease (PLP), which cleaves the first two protein products (leader protein p28 and p65 homologue). Within the nsp1 of MHV, two domains with papain-like protease activity (PLP1 and PLP2) have been mapped, (Kanjanahaluethai et al (2000) J. Virol 74(17):7911-21) which are also conserved with Bovine, transmittable gastroenteritis virus (TGV) and Human 229E coronaviruses. However, by sequence alignment with the SARS nsp 1, we identified only one PLP domain containing the catalytic residues Cys833 and His994.

Nsp2 is the chymotrypsin-picornavirus 3C-like protease (3CLp), which is responsible for the post-translational processing of the other 12 proteins, most of them cleaved at Q/A or Q/S sites. (Ziebuhr et al (1999) J. Virol 73(1):177-85). It also performs autoproteolytic activity. The principal catalytic residues are well conserved with other coronaviruses and are located at position His41 and Cys145. Furthermore, even the conserved aminoacids Tyr161 and His163, which are believed to be involved in substrate recognition and to be indispensable for proteolytic activity, (Hegyi et al (2002) J. Gen Virol 83(Pt3):581-593) were found in the sequence of the SARS 3CLp.

The invention includes the orflab sequence of SEQ ID NO: 9960 and the orfla sequence of SEQ ID NO: 9961, including fragments, variants, homologs, etc. thereof.

The Structural Region

Analysis of the nucleotide sequence at the 3′ part of the SARS genome identified 12 predicted open reading frames. They are coded within 8.2 kb and comprise the four structural proteins S, E, M and N, common for all coronaviruses and eight predicted ORFs, which are specific for this virus (FIG. 18). SARS-specific IG sequences upstream of most ORFs (FIGS. 17 & 18) suggest that most genes are likely to be transcribed independently. Interestingly, sequences identical to the group 2 IG are also present at the end of the RNA leader and in front of the Matrix encoding gene and of ORF 10.

The spike is a type I glycoprotein, which forms the large spikes on the surface of the virion and is responsible for receptor-binding and membrane fusion. (Gallagher (2001) Adv Exp Med Biol 494: 183-92). The protein is 1255 residues long with 17 predicted N-glycosylation sites. It has a 13aa leader peptide and a 17 aa C-terminal membrane anchoring sequence (1202-1218). Some (MHV, HCoV-OC43, AIBV and BCoV), but not all (TGV, FIPV, HCoV-229E) coronavirus spike proteins are proteolytically cleaved in two subunits, S1 and S2. S1 is supposed to form the bulbous head, which stays non-covalently linked to the C-terminal membrane anchor. Cleavage is mediated by a basic aminoacid sequence, which resembles the consensus sequence for a furin cleavage site. (Garten et al., Biochimie 1994; 76(3-4): 217-225). However, in case of this SARS virus isolate, we were not able to identify such a sequence, implicating that the S protein of this SARS virus isolate is unlikely to be cleaved during maturation. Secondary structure predictions indicated that the global architecture of the spike protein is conserved within all known coronaviruses. The S1 domain is mainly formed by beta sheets and likely adopts a globular fold, while in the S2 domain extensive alpha helical regions are predicted. In addition, the LearnCoil-VMF program, specifically designed to identify coiled-coil-like regions in viral membrane-fusion proteins, predicts two coiled-coils within S2, spanning aminoacids 900-1005 and 1151-1185, respectively (FIG. 19). Both coiled-coil regions contain a leucine-zipper motif, which is also present in the spikes of all coronaviruses. Leucine zippers are known to promote protein oligomerization; since the spike proteins of TGV and MHV form hetero-trimers, (Delmas et al, J Virol 1990; 64(11):5367-5375) (Godeke, et al., J Virology 2000; 74(3):1566-1571) it is conceivable that in SARS leucine zippers play a role in promoting and/or stabilizing a similar quaternary structure. The spike protein plays a major role in the biology of coronaviruses because the S1 domain contains the receptor-binding domain and the virus neutralizing epitopes, while the S2 domain is involved in the process of membrane fusion, which is essential for virus infectivity. As expected, multiple sequence alignment of different spike proteins showed a major degree of variability within the S1 domain, whereas S2 is more conserved.

The envelope protein E is a very short polypeptide of 76 aa, involved in the morphogenesis of the virion envelope. (Godet et al., Virology 1992; 188(2):666-675). Computer analysis predicts a long transmembrane domain close to the N-terminus and two N-glycosylation sites. The level of aminoacid similarity with other coronaviruses is very low and the best homology is with the small envelope protein of the transmissible gastroenteritis virus (TGV).

The matrix glycoprotein (M) is a 221-residue polypeptide with a predicted molecular weight of 25 kDa. Computer analysis predicts a topology consisting of a short aminoterminal ectodomain, three transmembrane segments and a carboxyl terminus located at the interior side of the viral envelope. In analogy with the matrix glycoprotein of TGV, that of the avian infective bronchitis virus (AIBV) and that of the hypervirulent MHV-2 strain the SARS M glycoprotein is N-glycosylated at the N-terminus. SARS M protein shows highest similarity to group 2 viruses (Table 11).

Finally, the nucleocapsid protein N is a 397-residue-long phosphoprotein that interacts with viral genomic RNA to form the nucleocapsid. The level of conservation with other coronaviruses is low, ranging from 26.9% identity with the HCoV-229E to 37.4% identity to the Bovine coronavirus (BcoV) (Table 11). Epitope analysis of the nucleocapsid protein has been carried out (Li et al. (2003) Geno Prot & Bioinfo 1:198-206) in which the epitope site at the C terminus of the protein was located as SEQ ID NO: 7394 (amino acids 371-407 of SEQ ID NO: 6052).

In addition to the above fundamental proteins, many viruses express a set of other peptides, which are generally dispensable for viability, but can influence the infectivity potential of the virus. (de Haan et al., Virology 2002; 296(1):177-189). These proteins are generally conserved within members of the same serogroup, but differ profoundly among the groups. For this reason, they are generally referred to as group-specific proteins (FIG. 11). Members of the group 1, represented here by HcoV-229E, have two group-specific genes located between the S and E genes and sometimes one or two ORFs downstream of the N gene, preceding the 3′ UTR region of the genome. Viruses of the group 2, with MHV as prototype, have two group-specific genes (2a and HE) between ORF1b and S, as well as other two between S and E genes. Finally, the group 3 viruses, represented by the prototype AIBV, have two group-specific genes between S and E and other two between the M and N genes.

With the exception of the hemagglutinin esterase HE, for which hemagglutinating and acetyl-esterase enzymatic activities have been demonstrated, all the other group-specific ORFs encode proteins whose role has not yet been established.

Interestingly, the arrangement of specific genes in the SARS genome is peculiar and the predicted ORFs do not display any significant homology with ORFs present in the other coronaviruses, nor with any other known protein from different organisms. Like viruses of the group 1 and 3, SARS lacks the HE hemagglutinin and does not contain ORFs between the ORF1b and the S gene. Furthermore, two predicted ORFs (ORF3 and ORF4) are encoded in the region between S and E, and superimpose for most of their length. ORF3 has an IG sequence 2 bp upstream of the ATG start codon. In contrast to the other groups, SARS contains five predicted ORFs in the region between M and N genes. ORF7 is located 10 bases downstream of the stop codon of M gene, and has an IG sequence 155 nucleotides upstream from the ATG start codon. Similarly, ORF8 and ORF10 present an IG right upstream of their ATG start codons. On the other hand, the 5′ ends of ORF9 and ORF11 shortly superimpose with the flanking genes, and for this reason they do not need an IG to activate transcription. ORF12 totally superimposes with the N gene and shares very low homology with a 22 kDa protein of the MHV virus, coded in the corresponding region.

Despite the absence of indications of possible localization and function deriving from sequence similarity, ORF3, ORF7 and ORF8 contain hydrophobic segments, suggesting association with membrane structures. In addition, ORF3, the longest among the SARS specific gene, is the only one that encodes for a peptide containing a high number of predicted O-glycosylation sites (Table 11). Predicted N-glycosylation sites have been identified in ORF3, ORF11 and ORF12.

Two shorter ORFs in the non-structural regions are SEQ ID NOS: 9965 and 9966. The invention includes polypeptides with these sequences, and also fragments, variants, etc.

Phylozenetic Analysis

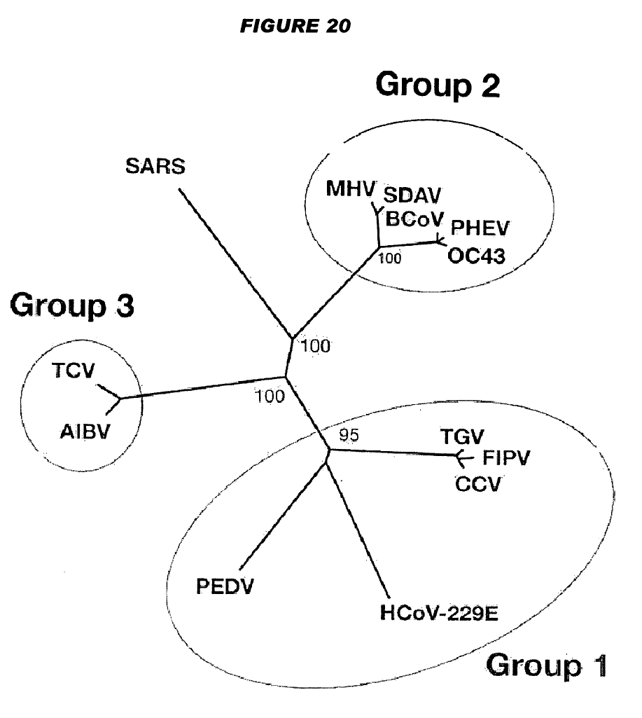

The substitution frequency within 922 conserved bases from the pol gene of eleven coronaviruses from the three different serogroups has been used in the past to show that the variability within members of each serogroup is much smaller than between members of different serogroups, confirming the previously described serological groupings. (Stephensen et al., Virus Res 1999; 60(2):181-9). We used the 922 bp region of the pol gene of SARS and aligned it with the same fragment from other 12 coronaviruses. The tree obtained showed that the SARS virus is distinct from the other three groups of coronaviruses (FIG. 20). Similar results were obtained using the full-length aminoacid sequences of pol, 3CL-protease and helicase from the replicase region and those of the spike and the matrix glycoproteins from the structural region (data not shown). These data confirmed that the entire genome of the SARS virus clusters in a new group (group 4) of coronavirus.

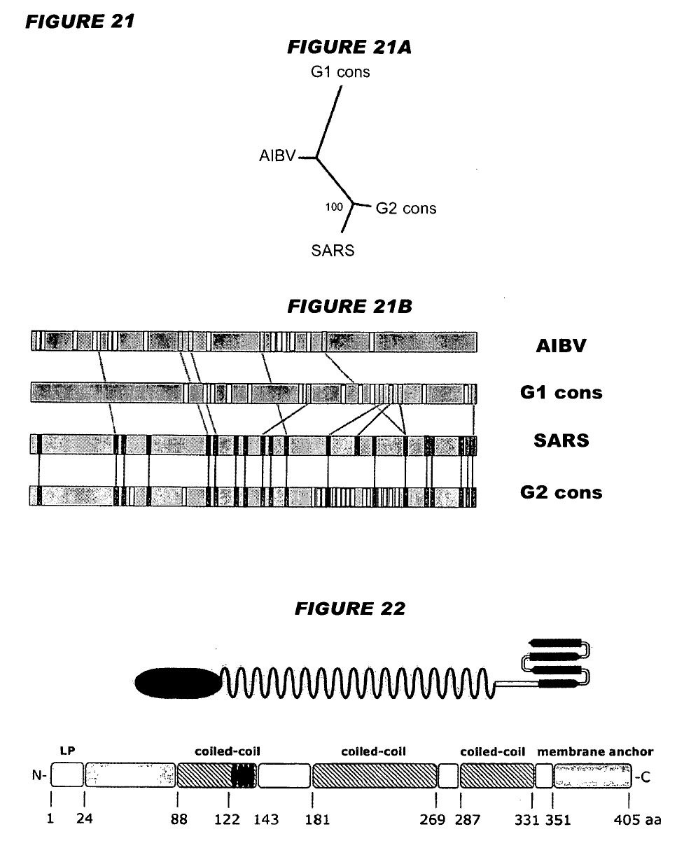

To gain more resolution for possible evolutionary relationships we performed the analysis using consensus sequences of predicted domains of the proteins. In particular, we generated consensus sequences of the S1 domain of the spike protein from the group 1 and group 2 and then we compared them to the S1 domain of the SARS spike. No consensus could be generated from the group 3 since only the spike protein of AIBV is known. Interestingly, the tree constructed from the alignment of SARS SI with the consensus generated from the two groups of spike proteins was different from that in FIG. 20, and showed a much closer relationship between SARS and group 2 coronaviruses (FIG. 21A). Further analysis showed that 19 out of the 20 cysteines present in the SARS S 1 domain are spatially conserved with the group 2 consensus sequence, while only five are maintained either within the group 1 and group 3 sequences (FIG. 21B). Given the fundamental role played by cysteines in protein folding, it is likely that the S1 domain of SARS and group 2 coronaviruses share a similar spatial organization.

Seguence Variability Between SARS Coronaviruses

We compared the FRA sequence to the four complete SARS genomes available on the Web. A total of 30 mutations were detected. Nine of these mutations were silent while 21 resulted in aminoacid substitutions (Table 12). Within ORF1a, three silent and seven productive mutations were detected. In ORF1b, there were five silent and three productive mutations. One of the productive mutations was caused by two nucleotide substitutions resulting in a single aminoacid change. Five changes were located in the spike protein, four of these were productive and one silent. Two productive mutations were in ORF3 and in the matrix glycoprotein M. One productive mutation each was in ORF10 and in the nucleocapsid protein N.

The overall difference between FRA and TOR2 was of nine nucleotides resulting in two silent mutations and seven aminoacid changes. The difference between FRA and Urbani is 12 nucleotides, which result in five silent mutations and seven aninoacid changes. For CUHK 16 nucleotides were different, five of which were silent mutations. For FRA and HKU 14 nucleotide changes resulted in four silent and nine productive mutations.

### Example 2

### Production, Inactivation and Purification of Whole SARS Virus Using MCS Chromatography Resin Purification Followed by Density Gradient Ultracentrifugation

A SARS isolate FRAI (EMBL: AY310120) was passaged on VERO cells that were cultivated in DMEM (Gibco: Cat No. 21969-035, Lot No. 3078864), Penicillin/Strep (Gibco: Cat No. 15070-063, Lot No. 1120042), and 3% FCS (Gibco: Cat No. 10270-106, Lot No. 40F6130K) at 37° C., 5% CO₂. Trypsin (Gibco: Cat No. 25300-054, Lot No. 3078729) was used for detaching the cells.

For virus production the third passage was used for inoculation of VERO cells at a moi of ˜0.1. Cells were incubated with the virus for 1 h at 37° C. in infection medium (DMEM without PS, FCS); after 1 h cells were washed twice and further incubated at 37° C. for 48 h in the presents of 3% FCS and antibiotics. The supernatant was harvested 48 hours post infection (p.i.) and precleared by centrifugation at 3000 rpm at 4° C. for 10 min.

The SARS virus was inactivated by (β-propiolactone (BPL) treatment (1:2000) for 18 h at 4° C., followed by 3 h at 37° C. Testing the virus on successful inactivation, VERO cells were incubated with 10 ml BPL treated supernatant for 4 days at 37° C.; subsequentially, the supernatant was transferred to a fresh VERO cell culture and further incubated for another 4 days. Cells were checked for cytopathic effect (CPE).

200 ml of the BPL-inactivated SARS virus harvest was then clarified using a 0.65 μm-pore-size filter (47 mm diameter) to pass virus particles and retain cell debris. The filter unit was connected to a Masterflex pump, which accomplished a consistent flow rate of 40 ml/min.

A. MCS Chromatography Purification Step

The filtered virus suspension was then subjected to MCS chromatography. The MCS column was prepared as follows. 27 ml slurry led to 14 ml sedimentated resin which Was packed using a Gotec Superformance Column (diameter 1.0 cm, height 15.7 cm, volume 12.33 ml). 1% of the column volume of a 1% acetone solution was injected to the column and the column was run with a flow of 100 cm/h. The HETP, N and A, values were then calculated as HETP: 0.056 cm, N/m: 1790 and Aₛ=1.20.

The amount of proteins in the purified solution after the MCS chromatography step were assessed with a bicinchoninic acid (BCA) method (Interchim) (see, e.g., http://www.piercenet.com/files/bca.pdj) and electrophoresis.

SDS-PAGE was done in accordance to Laemmli, Nature (1970) 227:680-685. Samples for SDS-PAGE were diluted to a protein concentration of 77 μg/ml. Different protein concentrations were loaded depending on the gel types used (10/12/15 Wells, Novex/Invitrogen):

| | Protein Concentration | | | | |
|---|---|---|---|---|---|
| Number of Wells | in the Dilution | Load | | Protein/Well | |
| 10 Wells | 77 μg/ml | 20 | μl | 1 | μg |
| 12 Wells | 77 μg/ml | 15-20 | μl | 0.75-1 | μg |
| 15 Wells | 77 μg/ml | 10 | μl | 0.5 | μg |

Samples for use in a reducing SDS-PAGE were prepared as, follows:

| | |
|---|---|
| | 26 μl sample or diluted sample |
| | +10 μl NuPage Sample Buffer (4×) SDS NP0003 |
| | +4 μl TCEP Bondbreaker Solution 77720 |
| | (1:2 in MilliQ water) |
| Final Volume: | 40 μl |

The samples were heated for 10 minutes at 70° C. or left at room temperature for 1 hour (leaving the samples at room temperature prevents the M protein of Corona Virus to coagulate/forming complexes), and then centrifuged for approximately one minute at 14,000 rpm in a table top centrifuge.

Markers for use on the gel were prepared as follows. Gel bands containing less than 1 μg of proteins were easily visualised with the silver staining procedure using the Silver Staining Kit Protein, Plus One Staining Protocol (Pharmacia Biotech).

Western blotting was performed as follows. A semi-dry blotting technique was used to transfer the proteins from the SDS gel to a nitrocellulose membrane. The transfer was performed with a current of 0.8 mA/cm² for 1 hour. A rabbit polyclonal antibody against SARS virus was used to perform the immuno probing using the Western Breeze, Novex Chromogenic Western Blot Immunodetection Kit (Novex/Invitrogen).

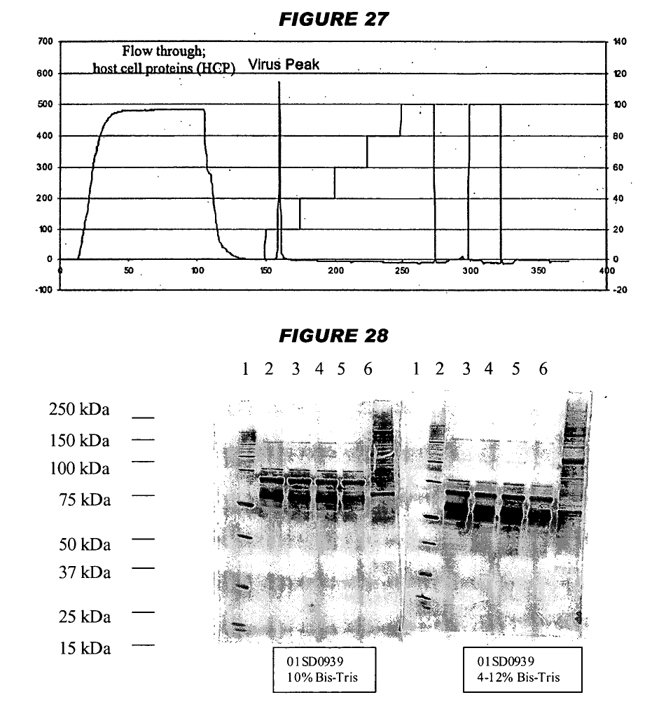

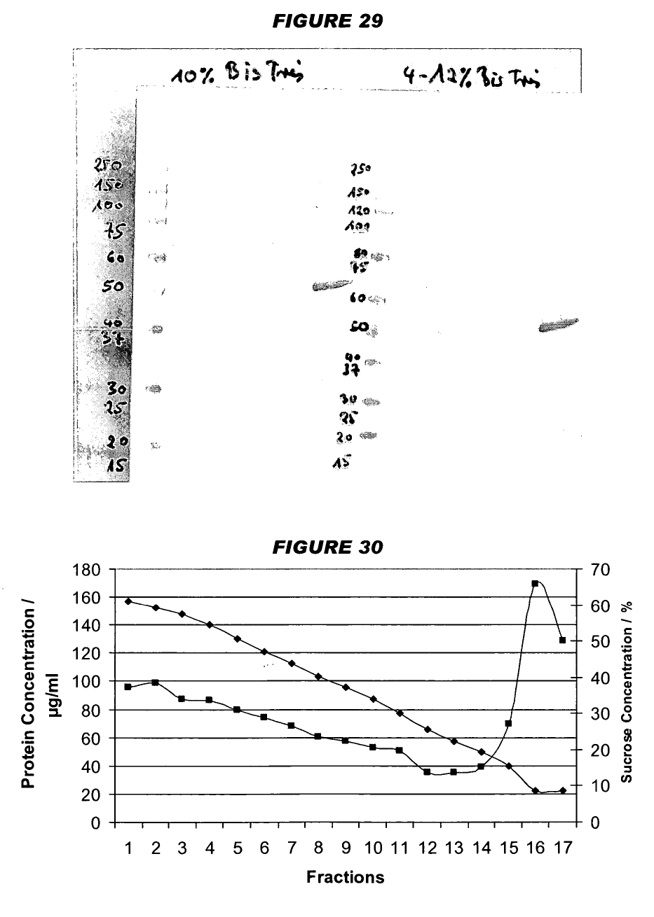

The chromatogram of the inactivated SARS MCS capture step is depicted in FIG. 27. To estimate purity, MCS chromatography fractions were analysed by silver staining on NuPage 10% or 4-12% Bis-Tris-Gel (Novex) under reduced conditions, heated for 10 minutes at 70° C. (FIG. 28). THe fractions were also analysed under the same conditions by western blot (FIG. 29) to estimate using PAK 11/03 SARS Cov 270603 neutralizing titer 1:512 (this antibody was used for this and subsequent western blots). Purity estimates are as follows:

| | Volume/ | [Protein]/ | Total Protein/ | Step Recovery |
|---|---|---|---|---|
| Sample | ml | μg/ml | mg | Protein/% |
| Corona Harvest | 100 | 2547.6 | 254.76 | 100 |
| After | 100 | 2440.3 | 244.03 | 95.8 |
| Filtration = | | | | |
| Load | | | | |
| Flow Through | 85 | 2321.4 | 197.32 | 77.5 |
| Wash | 49.32 | 468.5 | 23.11 | 9.1 |
| Peak 1 | 12.12 | 252.7 | 3.062 | 1.2 |
| Total Recovery | — | — | 464.4 | 86.5 |

B. Density Gradient Ultracentrifugation Step

The eluted SARS virus fraction was then subjected to density gradient ultracentrifugation with a swinging bucket rotor to further purify the inactivated virus. 3 ml of MCS peak fraction were loaded onto a linear gradient (15-60% sucrose; 17 ml 15% and 17 ml 60% sucrose in gradient mixer). The separation was performed with a Beckman SW 28 rotor at 20,000 rpm for 2 hours.

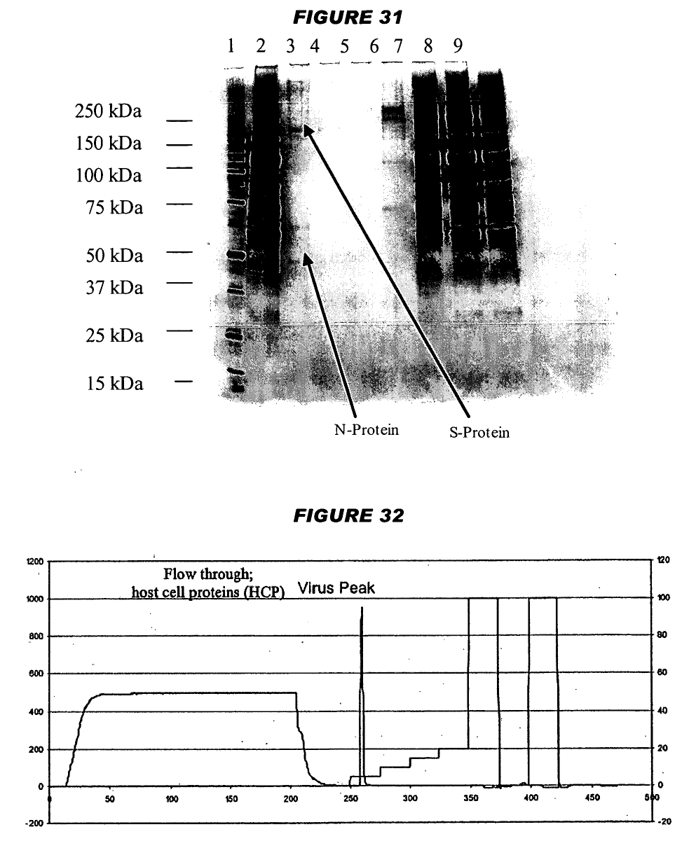

The content of sucrose and protein in the linear density gradient ultracentrifugation fractions are depicted in the following table, the graph in FIG. 30 and the estimation of purity in FIG. 31:

| Fraction | Fraction Size/ml | [Sucrose]/% | [Protein]/μg/ml |
|---|---|---|---|
| 1 | 2 | 61 | 96.12 |
| 2 | 2 | 59.4 | 98.62 |
| 3 | 2 | 57.5 | 87.63 |
| 4 | 2 | 54.5 | 86.91 |
| 5 | 2 | 50.5 | 79.9 |
| 6 | 2 | 47.2 | 74.3 |
| 7 | 2 | 43.7 | 68.05 |
| 8 | 2 | 40.2 | 60.43 |
| 9 | 2 | 37.2 | 57.38 |
| 10 | 2 | 34 | 53.12 |
| 11 | 2 | 30 | 50.63 |
| 12 | 2 | 25.7 | 35.02 |
| 13 | 2 | 22.4 | 35.33 |
| 14 | 2 | 19.5 | 39.25 |
| 15 | 2 | 15.5 | 69.79 |
| 16 | 2 | 8.5 | 169.03 |
| 17 | 2 | 8.5 | 128.96 |

The protein concentration of fraction 11 (FIG. 31 SDS-gel) was measured again against a standard curve prepared in 30% sucrose and lead to a protein concentration of 3.67 μg/ml (0.05 μg on the gel). The M protein appears to be missing in this preparation possibily due to sample treatment procedure (heated samples).

There may be discrepancies in the protein concentration measurements in Table 2 due to sucrose interface with this assay.

### Example 3

### Production, Inactivation and Purification of Whole SARS Virus Using MCS Chromatography Resin Purification Followed by Density Gradient Ultracentrifugation

Inactivated SARS virus was prepared as described in Example above.

A. MCS Chromatography Purification Step

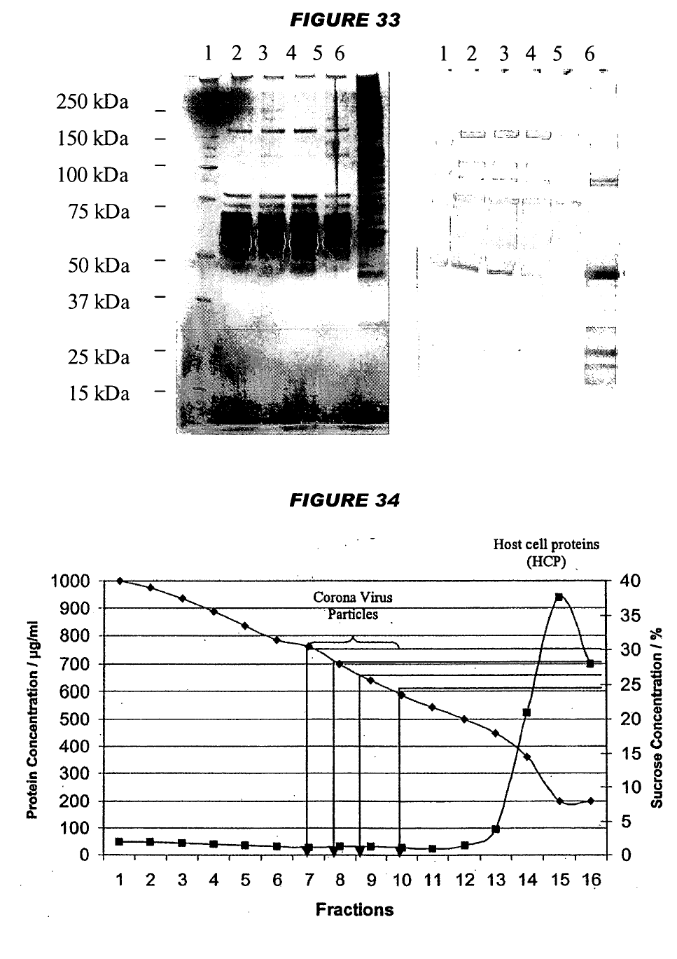

In this example, 200 ml of inactivated SARS virus harvest were subjected to MCS chromatography. The chromatogram of the capture step of inactivated SARS virus purification with MCS is depicted in FIG. 32, the protein recovery in the following table and the estimation of purity in FIG. 33:

| | Volume/ | [Protein]/ | Total Protein/ | Step Recovery |
|---|---|---|---|---|
| Sample | ml | μg/ml | mg | Protein/% |
| Corona | 200 | 2239.2 | 447.83 | 100 |
| Virus Harvest | | | | |
| After | 200 | 2245.1 | 449.02 | 100.3 |
| Filtration = | | | | |
| Load | | | | |
| Flow Through | 185 | 2126.3 | 393.37 | 87.8 |
| Wash | 49.32 | 450.1 | 22.2 | 5.0 |
| Peak 1 | 4.43 | 1245.6 | 5.52 | 1.2 |
| Total Recovery | — | | 421.08 | 93.7 |

Density Gradient Ultracentrifugation Step

3.5 ml of MCS peak fraction were then loaded onto a linear gradient (15-40% sucrose: 16 ml 15% and 16 ml 40% sucrose in gradient mixer). The separation was performed with a Beckman SW 28 rotor at 20,000 rpm for 2 hours.

The content of sucrose and protein in the linear density gradient ultracentrifugation fractions are depicted in the following table and the graph in FIG. 34:

| Tube | Fraction Size/ml | [Sucrose]/% | [Protein]/μg/ml |
|---|---|---|---|
| 1 | 2 | 40 | 45.86 |
| 2 | 2 | 39 | 45.68 |
| 3 | 2 | 37.5 | 44.14 |
| 4 | 2 | 35.5 | 37.82 |
| 5 | 2 | 33.5 | 34.48 |
| 6 | 2 | 31.5 | 31.76 |
| 7 | 2 | 30.5 | 29.49 |
| 8 | 2 | 28 | 30.87 |
| 9 | 2 | 25.5 | 31.7 |
| 10 | 2 | 23.5 | 26.74 |
| 11 | 2 | 21.75 | 23.58 |
| 12 | 2 | 20 | 35.33 |
| 13 | 2 | 18 | 96.38 |
| 14 | 2 | 14.5 | 523.79 |
| 15 | 2 | 8 | 941.97 |
| 16 | 2 | 8 | 696.7 |

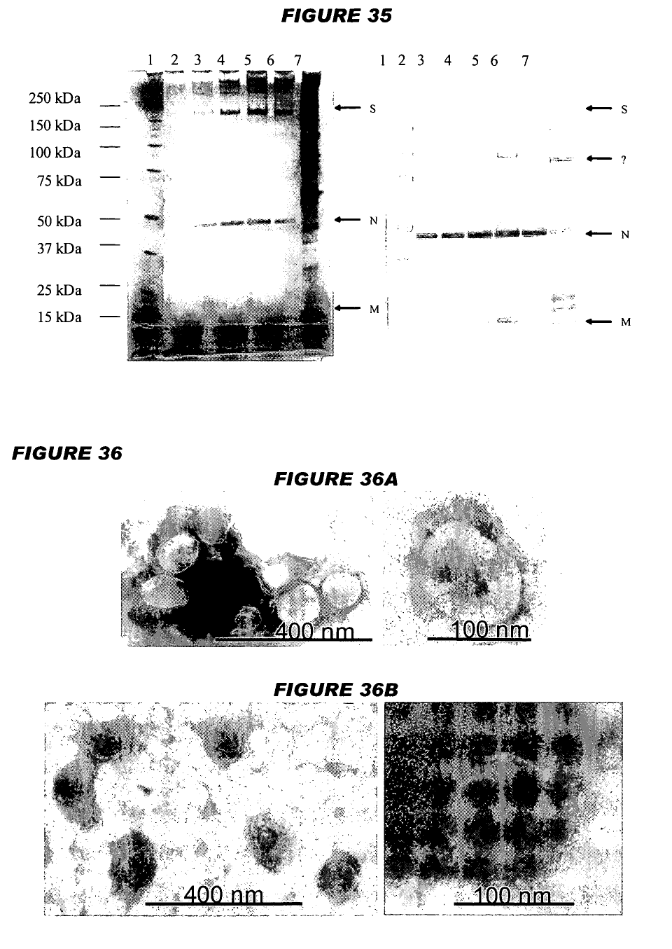

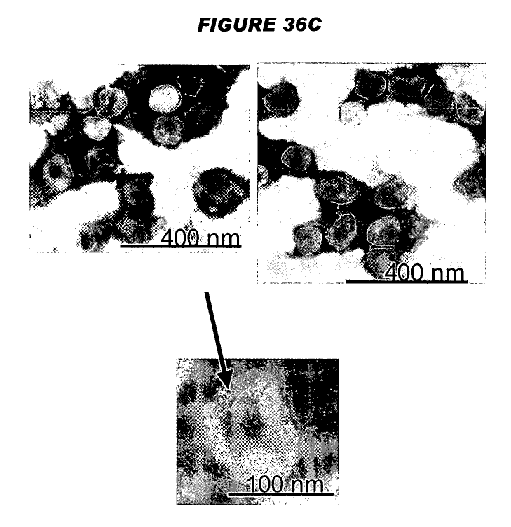

Protein recovery is shown in the following table and the estimation of purity is shown in FIG. 35. Electron Micrograph pictures of density gradient fractions 8, 9 and 10 are shown in FIG. 36:

| | | Protein/ | Total | Step |
|---|---|---|---|---|
| Step | Volume/ml | μg/ml | Protein/mg | Protein % |
| Load | 3.5 ml | 1245.6 | 4359.6 | 100 |
| Bulk Protein Fractions | 3.5 ml | 720.8 | 4324.9 | 99.2 |
| Viral Peak Fraction | 8 ml | 29.7 | 237.6 | 5.5 |
| Total Recovery | | | 4562.5 | 104.7 |

### Example 4

### Mouse Immunization with Inactivated SARS Virus

Mice were immunized subcutaneously on days 0, 14, and 28 with 5 μg BPL-inactivated SARS-CoV particles (BPL-SARS-CoV), either alone or together with Alum or MF59 as adjuvants. Serum was collected on days 0 (pre-immunization), 13 (post 1st immunization), 28 (post 2nd), and 35 (1 week post 3rd immunization). Neutralizing antibodies were assessed for blocking SARS-CoV infection of Vero cells in vitro. After 3 immunizations, neutralization titers were in the range 1:100-1:1000, which are levels similar to those present in the serum of SARS convalescent patients. As shown in the following table, the non-adjuvanted vaccine induced neutralizing antibody after the third immunization, and potency of this vaccine was enhanced significantly by including the adjuvants, with neutralizing antibody appearing after then 2nd immunization and overall titers increasing after then 3rd immunization:

| | Neutralization Titer | | | |
|---|---|---|---|---|
| Immunogen | pre | post 1st | post 2nd | post 3rd |
| BPL-SARS-CoV + MF59 (5 μg) | <1:20 | <1:20 | 1:158 | 1:630 |
| BPL-SARS-CoV + Alum (5 μg) | <1:20 | <1:20 | 1:67 | 1:612 |
| BPL-SARS-CoV (5 μg) | <1:20 | <1:20 | <1:20 | 1:71 |
| PBS | <1:20 | <1:20 | <1:20 | <1:20 |

### Example 5

### Balb/cMouse Immunization with Inactivated SARS Virus

A Balb/c mouse model for SARS infection has been developed (Subbarao et al. (2004), J. Virol., 78:3572-77. In this model, Balb/c mice are inoculated intranasally with 10⁴ TCID₅₀ of virus. At 48 hours post-inoculation, a 2-log increase in the TCID₅₀ virus titer can be detected in the lungs of infected mice. While virus replication is readily detected, the mice do not show any SARS disease symptoms and spontaneously clear the virus one week after inoculation. A decrease in virus titer in previously-immunized animals as compared to control animals demonstrates a protective effect of the vaccine being evaluated.

In this example, four Balb/c mice per group are immunized three times with 5 μg BPL inactivated SARS-CoV (days 0, 14, 28) either alone or in combination with MF59 and challenged with 10⁴ TCID₅₀ of SARS-CoV on day 43. Two days following virus challenge the mice are euthanized and SARS-CoV is quantified from nasal turbinates (NT) and lungs and the mean virus titer for each mouse is measured. Control groups received PBS alone, or an influenza virus vaccine (FLU) with or without MF59 adjuvant. Data were as follows (see also FIG. 51), where four mice were tested per group and virus titers are expressed as log₁₀ TCID₅₀ per gram of tissue:

| | Virus replication | | Virus replication in nasal | |
|---|---|---|---|---|
| | in lungs of | | turbinates of | |
| | challenged mice | | challenged mice | |
| | # infected/ | Mean (± SE) | # infected/ | Mean (± SE) |
| Immunogen | # tested | virus titer | # tested | virus titer |
| PBS | 4/4 | 6.3 ± 0.3 | 3/4 | 2.8 ± 0.35 |
| MF-59 alone | 4/4 | 6.1 ± 0.13 | 3/4 | 3.0 ± 0.38 |
| FLU vaccine (5 μg) | 4/4 | 6.3 ± 0.07 | 3/4 | 2.9 ± 0.36 |
| FLU vaccine (5 μg) + MF-59 | 4/4 | 6.0 ± 0.19 | 4/4 | 3.0 ± 0.11 |
| BPL-SARS-CoV (5 μg) | 1/4 | 1.6 ± 0.13* | 0/4 | Not detected** |
| BPL-SARS-CoV (5 μg) + MF-59 | 0/4 | Not detected* | 0/4 | Not detected** |

Two-tailed Student's t-test, compared to PBS-immunized mice, showed:
*P < 0.00001 or
**P = 0.025

As shown, virus could not be detected in the BPL-SARS-CoV immunized mice. The lower limit of detection of infectious virus in a 10% w/v suspension of lung homogenate was 1.5 log₁₀TCID₅₀/gm, and in a 5% w/v suspension of nasal turbinates the limit was 1.8 log₁₀TCID₅₀/gm. Viral titers in the immunized mammals were thus below these threshold values.

Thus the inactivated SARS-CoV vaccine was very efficient at preventing virus infection, as only one of eight mice immunized with the vaccine, either with or without MF59 adjuvant, was infected. Similar protection was not observed in control groups of PBS diluent, MF59 adjuvant, or influenza virus vaccine with or without adjuvant.

Neutralization titers of sera taken from the animals in the challenge study were assessed at two weeks post-1st, one week post-2nd, and one week post-3rd immunization. Mice immunized with the vaccine with MF59 adjuvant had already developed a neutralization titer of 1:71 after the 2nd immunization, which increased to 1:588 after the 3rd immunization, whereas mice receiving the unadjuvanted vaccine did not have any neutralizing activity post-2nd and a neutralization titer of 1:64 post-3rd immunization. Sera from mice in each of the control groups did not show any neutralization activity. These data clearly demonstrate not only the ability of the inactivated SARS-CoV vaccine to induce protective levels of SARS neutralizing antibodies, but also a beneficial effect of formulating the vaccine with adjuvant for elevated neutralization titers.

### Example 6

### Preparation of OMV Comprising SARS Viral Antigens

E. coli were transfected with a plasmid of interest (encoding a SARS viral antigen). Single colonies harbouring the plasmid of interest were grown overnight at 37° C. in 20 ml of LB/Amp (100 μg/ml) liquid culture. Bacteria were diluted 1:30 in 1.0 L of fresh medium and grown at either 30° C. or 37° C. until the OD₅₅₀ reached 0.6-0.8. Expression of recombinant protein was induced with IPTG at a final concentration of 1.0 mM. After incubation for 3 hours, bacteria were harvested by centrifugation at 8 000×g for 15 minutes at 4° C. and resuspended in 20 ml of 20 mM Tris-HCl (pH 7.5) and complete protease inhibitors (Boehringer-Mannheim™). All subsequent procedures were performed at 4° C. or on ice.

Cells were disrupted by sonication using a Branson Sonifier 450 and centrifuged at 5 000×g for 20 min to sediment unbroken cells and inclusion bodies. The supernatant, containing membranes and cellular debris, was centrifuged at 50000 g (Beckman Ti50, 29 000 rpm) for 75 min, washed with 20 mM Bis-tris propane (pH 6.5), 1.0 M NaCl, 10% (v/v) glycerol and sedimented again at 50000 g for 75 minutes. The pellet was resuspended in 20 mM Tris-HCl (pH 7.5), 2.0% (v/v) Sarkosyl, complete protease inhibitor (1.0 mM EDTA, final concentration) and incubated for 20 minutes to dissolve inner membrane. Cellular debris was pelleted by centrifugation at 5000 g for 10 min and the supernatant centrifuged at 75000 g for 75 minutes (Beckman Ti50, 33000 rpm). Outer membrane vesicles were washed with 20 mM Tris-HCl (pH 7.5) and centrifuged at 75 000×g for 75 minutes or overnight. The OMV was finally resuspended in 500 μl of 20 mM Tris-HCl (pH 7.5), 10% v/v glycerol. Protein concentration was estimated by standard Bradford Assay (Bio-Rad), while protein concentration of inner membrane fraction was determined with the DC protein assay (Bio-Rad). Various fractions from the isolation procedure were assayed by SDS-PAGE.

### Example 7

### Immunogenicity, Dose and Route Schedule for Recombinant Spike Protein in Mice

The immunogenicity, route and dosing of the recombinant spike proteins of the invention in mice may be assessed using the below detailed protocol. Preferably, the administered antigen will elicit neutralizing antibody titers at least in the range of 1/100- 1/1000. Increasing doses of antigen can be tested in the range from 5 to 20 μg of recombinant Spike antigen alone or mixed with an equal volume of MF59-citrate, administered SC or IM to anesthetized mice in 100 μl of inoculum. Groups of BALB/c mice, 6 per treatment are primed at day 0 and boosted at day 14 and 28.

| Group | Treatment | Dose/Route | Sampling interval | Number of mice |
|---|---|---|---|---|
| 1-3 | Rec-Spike protein | 20, 10, 5 μg/SC | 7, 21, 35, 42 d | 6 per dose level |
| 4-6 | Rec-Spike protein | 20, 10, 5 μg/SC | 7 | 6 per dose level |
| 7-9 | Rec-Spike protein | 20, 10, 5 μg/IM | 7, 21, 35, 42 d | 6 per dose level |
| 10-12 | Rec-Spike protein | 20, 10, 5 μg/IM | 7 | 6 per dose level |
| 13-15 | Rec-Spike - MF59 | 20, 10, 5 μg/SC | 7, 21, 35, 42 d | 6 per dose level |
| 16-18 | Rec-Spike - MF59 | 20, 10, 5 μg/SC | 7 | 6 per dose level |
| 19-21 | Rec-Spike - MF59 | 20, 10, 5 μg/IM | 7, 21, 35, 42 d | 6 per dose level |
| 22-24 | Rec-Spike - MF59 | 20, 10, 5 μg/IM | 7 | 6 per dose level |
| 25 | MF59 | NA/SC | 7, 21, 35, 42 d | 6 + 6 (sac d 7 and 42) |
| 27 | MF59 | NA/IM | 7, 21, 35, 42 d | 6 + 6 (sac d 7 and 42) |
| 29 | Saline | NA/SC | 7, 21, 35, 42 d | 6 + 6 (sac d 7 and 42) |
| 31 | Saline | NA/IM | 7, 21, 35, 42 d | 6 + 6 (sac d 7 and 42) |

This protocol can also be used to assess the Th1/Th2 profile of the specific immune response elicited by the recombinant Spike protein. Neutralizing and Spike-specific antibody titers will be assessed at days 7, 21, and 35; IgG2a vs IgG1 isotype of the Spike-specific antibodies will be determined at days 21 and 35; in vitro proliferation of lymph node and splenic T cells against the recombinant Spike protein will be determined at days 7 and 42, respectively; IFN-γ and IL-4 production by splenic T cell against the recombinant Spike protein from SARS-CoV will be assessed at day 42. Peripheral blood will be collected at days 7, 21, 35; lymph nodes cells at day 7, and spleen cells at day 42. Neutralizing and Spike-specific antibody titers and isotypes will be determined by inhibition of SARS-CoV infection of Vero cells and by ELISA, respectively. Proliferation of lymph node and splenic cells will be determined by ³[H]-Thymidine uptake. Frequencies of splenic IFN-γ and IL-4 producing T lymphocytes, will be determined by ELISPOT and FACS.

### Example 8

### Immunogenicity, Dosing and Route Schedule for Spike Proteins in Rabbits

The immunogenicity, route and dosing of the recombinant spike proteins of the invention in rabbits may be assessed using the below detailed protocol. Increasing doses can be tested in the range from 5 to 40 μg of recombinant Spike antigen alone or mixed with an equal volume of MF59-citrate, administered SC or IM to anesthetized animals in 200 μl of inoculum. Groups of New Zealand white female rabbits, 10 per treatment, will be immunized as shown in the table 20 below. The animals will be primed at day 0 and boosted at days 14 and 28. Peripheral blood will be collected at days 7, 21, and 35. Neutralizing and Spike-specific antibody titers will be determined by inhibition of SARS-CoV infection of Vero cells and by ELISA, respectively.

| Group | Treatment | Dose/Route | Sampling interval | Number of rabbits |
|---|---|---|---|---|
| 1-4 | Full-length Spike protein | 40, 20, 10, 5 μg/SC | 7, 21, 35 d | 10 per dose level |
| 5-8 | Full-length Spike protein | 40, 20, 10, 5 μg/IM | 7, 21, 35 d | 10 per dose level |
| 9-12 | Truncated Spike protein | 40, 20, 10, 5 μg/SC | 7, 21, 35 d | 10 per dose level |
| 13-16 | Truncated Spike protein | 40, 20, 10, 5 μg/IM | 7, 21, 35 d | 10 per dose level |
| 17-20 | Full-length Spike protein - MF59 | 40, 20, 10, 5 μg/SC | 7, 21, 35 d | 10 per dose level |
| 21-24 | Full-length Spike protein - MF59 | 40, 20, 10, 5 μg/IM | 7, 21, 35 d | 10 per dose level |
| 25-28 | Truncated Spike protein - MF59 | 40, 20, 10, 5 μg/SC | 7, 21, 35 d | 10 per dose level |
| 29-32 | Truncated Spike protein - MF59 | 40, 20, 10, 5 μg/IM | 7, 21, 35 d | 10 per dose level |
| 33 | MF59 | NA/SC | 7, 21, 35 d | 10 |
| 34 | MF59 | NA/IM | 7, 21, 35 d | 10 |
| 35 | Saline | NA/SC | 7, 21, 35 d | 10 |
| 36 | Saline | NA/IM | 7, 21, 35 d | 10 |

### Example 9

### Immunogenicity and Dose Schedule for Recombinant Spike in Ferrets

The immunogenicity and dosing of the recombinant spike proteins of the invention in ferrets may be assessed using the below detailed protocol. Three groups of ferrets, 6 for treatment, will be immunized with recombinant SARS-CoV Spike protein from CHO cell lines, alone or mixed with an equal volume of MF59-citrate, administered SC to anesthetized animals in 200 μl of inoculum. The recombinant Spike protein vaccine will be tested at the dose eliciting the highest neutralizing antibody titers in mice at day 35 after the second boost. The animals will be primed at day 0 and boosted at day 14 and 28. Peripheral blood will be collected at days 7, 21, and 35. Neutralizing and Spike-specific antibodies titers will be determined by inhibition of SARS-CoV infection of Vero cells and by ELISA, respectively.

| | | | Sampling | Number |
|---|---|---|---|---|
| Groups | Treatment | Dose/Route | interval | of ferrets |
| 1 & 2 | Rec-Spike protein | Y μg or 2Y μg/SC | 7, 21, 35 d | 6 |
| 3 & 4 | Rec-Spike protein + | Y μg or 2Y μg/SC | 7, 21, 35 d | 6 |
| | MF59 | | | |
| 5 | Saline | NA/SC | 7, 21, 35 d | 6 |

The 3 groups of ferrets, 6 animals per group, used for the immunogenicity studies above can then be used to assess efficacy of the recombinant Spike protein in protecting vaccinated animals from infection and/or disease. Anestethized animals will be challenged two weks after the last boost intratracheally with 10⁶ median tissue culture infectious dose unit (TCID₅₀) of the SARS-CoV Utah strain. Infection by SARS-CoV will be assessed by taking nasal, faringeal and rectal swabs from animals for 20 days after challenge as described (12). The presence of SARS-CoV in sample materials will be assessed by RT-PCR and infection assay of Vero cells. Animals will be monitored for clinical signs of SARS disease by assessing sleeping time, temperature, respiratory symptoms, diarrhea, body weight and survival. Protection will be determined by the magnitude and duration of virus shedding and by duration and severity of disease symptoms and percentages of surviving animals.

### Example 10

### Expression of Spike Protein for Vaccination

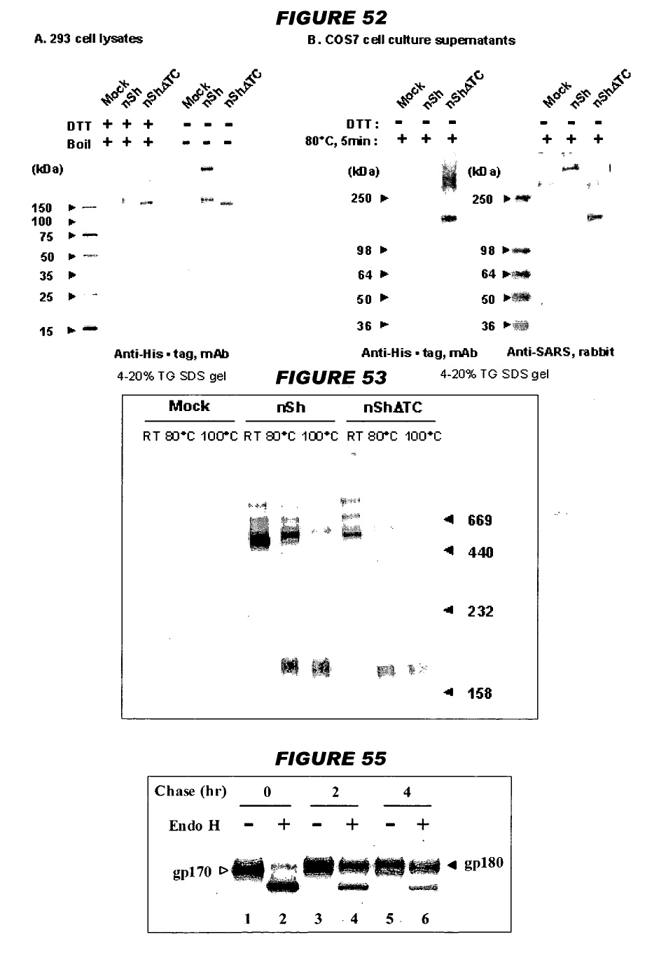

The SARS-CoV Spike glycoprotein was expressed in both full-length and truncated forms, using the nSh and nShATC pCMVIII constructs described above, both with hexahistidine tags. The vector constructs were evaluated for expression 48 hr after transfection into 293 cells and COS7 cells. The full-length Spike protein (nSh) was detected by western blot only in cell lysate, but not in culture media (FIG. 52).

The majority of SARS-CoV full-length Spike protein was expressed in transiently-transfected COS7 cells as a high molecular glycoprotein which ran at 540 kDa in non-reducing gels (FIG. 53). The gp540 is heat labile as indicated by the complete dissociation into monomeric forms (gp170 & gp180) by boiling, but it was resistant to DTT treatment. These data suggest that the recombinant Spike protein is noncovalently associated into a homotrimer (gp540). The presence of Spike protein in homotrimeric association also was confirmed in inactivated, purified SARS-CoV virion particles. Analysis of virion proteins by western blot under the same condition used for the characterization of recombinant Spike protein generated essentially identical results (FIG. 54).

### Example 11

### Spike Protein Processing

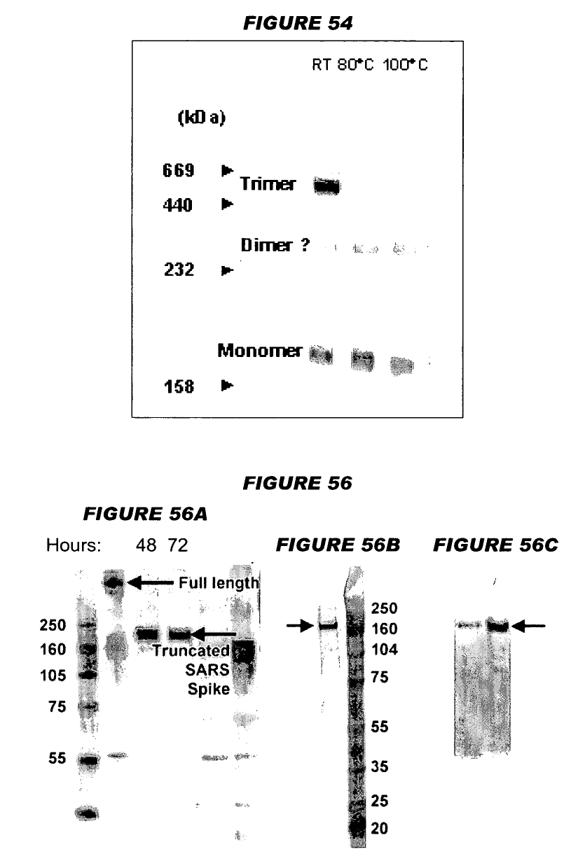

In order to characterize Spike protein processing, BHK-21 cells were infected with alphavirus replicon particles expressing the SARS-CoV full-length Spike. At 6 hoursr post-infection with an MOI of 5, infected cells were labeled for 1 hr with L-[³⁵S]methionine/cysteine and chased for up to 4 hours. The [³⁵S]-labeled spike protein was immunoprecipitated by anti-SARS rabbit serum and digested with Endo-H. Both digested and undigested proteins were analyzed by SDS-PAGE (4% polyacrylamide). As shown in FIG. 55, the full-length spike protein is synthesized as an Endo-H sensitive high-mannose glycoprotein (gp170, an ER form) that undergoes modification to an Endo-H resistant glycoprotein with complex oligosaccharides (gp180, a Golgi form). The conversion of gp170 into the gp180 form takes place within 2 hours (FIG. 56).

### Example 12

### High-Level Protein Expression

To develop a system for rapid expression of protein antigens, DNA transfection of 293 (human embryonic kidney) cells was used, to obtain milligram quantities of recombinant antigen. The most common method for culturing and transfecting 293 cells is in static or monolayer cultures. These procedures were modified by performing large-scale transfection of 293 cells in suspension and expanding the transfected cells in suspension culture for production of secreted or intracellular proteins. Several initial experiments were performed at the 100-milliliter scale cultures to determine optimum conditions, such as number of cells, type of transfecting reagent (FuGENE 6, Lipitoid or RO-1538) and the ratio of DNA to transfection reagent. Based upon pilot experiments, FuGENE 6 was the best transfecting reagent.

The kinetics of gene expression was compared to other viral envelope glycoproteins, and the data suggest that stable protein expression peaks around 72 to 96 hours post-transfection, depending upon the gene of interest, and then significantly decreases thereafter. Thus, using the optimum conditions, the transfection process was scaled from 100 ml to 4 liters. The 4 liter culture can be used for rapidly producing 2-10 milligrams of protein antigens. To facilitate antigen purification and also maximize the yield and recovery of the purified protein, transfection conditions were optimized by using serum-free medium.

Bulk transfection procedure has been used for the expression of truncated and full-length Spike antigens. The kinetics of expression for truncated form of the spike protein is presented in FIG. 56A. Expression of the truncated form of Spike protein peaked around 48 hrs and was stable until 72 hrs, therefore the cultures were harvested at 72 hrs post transfection.

Collected media were concentrated 20X and used for purification of truncated Spike protein by a very simple purification strategy where the truncated form of the spike was captured on GNA lectin followed by DEAE and ceramic hydroxyapatite column chromatography. The purified protein was analyzed on SDS-PAGE by silver stain (FIG. 56B) and also by western blot (FIG. 56C). Early efforts were able to purify the truncated form of the spike protein with >95% purity and approximately 50% recovery. The molecular mass of the truncated form of the Spike protein is approximately 170-180 kDa.

Full-length Spike protein was expressed in 293 cells using the bulk transfection strategy. The expression data suggest that, like the truncated form, expression peaked around 48 hrs post-transfection and remained stable until 72 hrs. However, contrary to the truncated form and as expected, full-length protein is not secreted, but rather is retained within the cells, as shown by the absence of any signal in western blots of cell culture supernatants. The full-length form of the protein was purified from Triton X-100 detergent-extracted cells. Full-length Spike protein was then captured on GNA lectin, followed by hydroxyapatite and SP chromatography. The calculated molecular mass of full-length spike protein is approximately 600 kDa, which is close to the theoretical mass for the trimer.

### Example 13

### SARS Virus Seed Cultures

A SARS-CoV reference seed virus propagated only in certified Vero cells will be used for the generation of the Master and Working Virus Seeds under GMP. A clinical specimen from the respiratory tract of a patient infected by the SARS-CoV is inoculated onto documented VERO cells, with certified culture media. Culture media containing the virus are harvested at 4 days post-infection and designated Passage 1 (P1). A second round of virus propagation is again performed in certified VERO cells with certified media, by inoculation of 1 ml per T-75 flask of 100 times diluted P1 virus. Culture supernatant was harvested at 3 days post-infection and stored at −80° C. as a P2 reference stock virus, without plaque purification.

Cell banks of Vero cells for further production of SARS-CoV are prepared from specific cell subsets that have not been used since the emergence of transmissible spongiform enephalopathies (e.g. since 1980). A research cell bank of these cells has been prepared using specified New Zealand-origin fetal bovine serum. From this research cell bank, a Master Cell Bank (MCB) is made under GMP conditions and using only specified and well-controlled media and supplements. The cell bank will is tested for absence of adventitious agents according to applicable US, EU, and international guidelines (see Points To Consider “Characterization of cell lines used to produce biologicals”, FDA/CBER 7/1993; ICH Q5D Draft 6 “Cell substrates”, Oct. 23, 1996; CPMP/ICH/294/95 “Note for Guidance on Quality of Biotechnological Products: Derivation and Characterisation of Cell Substrates Used for Production of Biotechnological/Biological Products (Step 4, 16. Jul. 1997); WHO final draft “Requirements for use of animal cells as in vitro substrates for the production of biologicals” Jul. 3, 1997). Tumorigenicity and identity testing is also required for this cell bank.

The reference virus is plaque-purified and expanded in certified Vero cells in the absence of FCS in order to generate Master and Working Seeds. Another option to help ensure purity and facilitate the assessment of safety of the Master Seed is to subject the SARS-CoV to pelleting and resuspension in PBS. The virus suspension is made up to 60% (w/w) sucrose with crystalline sucrose, transferred to a centrifuge tube and overlayed with 50, 40, 30, and 20% (w/w) sucrose solutions in PBS. The gradient is centrifuged for 72 h and then fractionated. The virus-containing fraction is diluted and the virions re-pelleted by ultracentrifugation. RNA from the virus pellet is isolated and transfected into certified Vero cells whereby the “infectious” positive-strand RNA will lead to the production of infectious virus, which can be plaque-purified and expanded to generate alternative Master and Working Seeds from purified virus RNA.

Viral seeds are tested for the absence of adventitious agents (see e.g. 21 CFR Revised as of Apr. 1, 1994, §630.35 Test for safety) and for identity, using a highly-specific neutralizing antiserum prepared from an independent source. Safety testing of viral seeds for vaccine purposes is done routinely by service laboratories. Broad-spectrum PCR testing can be used as an addition and/or alternative for testing.

### Example 14

### Scale-Up of Virus Production and Inactivation

A protocol for the production, inactivation, and purification of inactivated SARS-CoV with sufficient structural integrity to elicit protective neutralizing antibody responses in animal models involves: Vero cells are infected with virus at an M.O.I. of 0.01 in the absence of FCS and antibiotics; culture medium is collected, cleared by centrifugation, and inactivated with BPL, followed by confirmatory testing for complete inactivation; the inactivated material is filtered, subjected to MCS-column purification, and further purified by sucrose gradient centrifugation.

Several modifications and improvements can be developed when adapting this basic protocol to a larger scale for commercial use. Firstly, the cell culture and infection process can be adapted to roller bottles, as an intermediate step to allow rapid production for preliminary trials within existing BSL 3+ facilities. Full commercial production will typically use a fermentation process in a closed system, but a roller bottle system can be achieved more rapidly. The roller bottles do offer a true suspension culture system for Vero cells, which gives various technical and safety advantages over microcarrier cultures. Suspension cultures can be grown to any desired fermentation scale without interfering with the closed system between cell passages, as no trypsinization is required.

To scale up the infection process in roller bottles to 30-50 liters per batch, the optimum M.O.I. and harvesting periods for selected media and culture conditions should first be determined. For the larger scale, methods for harvesting and handling larger volumes of highly infectious material safely should be used, and so cell separation via centrifugation should be replaced by a method such as filtration through single-use filter cartridges.

The MCS-chromatography and the gradient purification steps described above can readily be scaled to a batch volume of up to 50 liters. For larger volumes, however, and for increased purity, ultrafiltration and sterile filtration steps will be used. Nuclease treatment to remove host cell DNA will also be included.

### Example 15

### Large Scale Analytical Methods

Analytical methods for the SARS coronavirus include virus titration methods, immunological and physico-chemical methods to quantitate and characterize the purified antigen (ELISA, PAGE, western blots using specific antisera against purified whole virus, etc.). Other analytical tests include: fast yield testing via asymmetric field flow separation and laser particle detection and counting; Western blot using specific antisera against individual viral proteins; and tests for residual host cell DNA.

Residual DNA testing is generally done by hybridization e.g. using a limit test. Such testing is performed according to methods already established and validated for other cell lines. As an alternative, the Threshold™ method may be used.

For producing specific antibodies, recombinant protein expression of all the ORFs from the structural and non-structural gene regions of the SARS-CoV is used. The ORFs can be cloned and expressed in E. coli and, if necessary, also in eukaryotic vectors such as baculovirus. This can provide sufficient amounts of purified soluble protein to immunize mice and rabbits to produce polyclonal and monoclonal antibodies against SARS proteins and to set up specific ELISA assays. Different expression vectors can be tested to maximize the yield of recombinant protein in a soluble form e.g. different vectors, one containing sequences coding for six N-terminal histidine residues and another containing a Glutathione-S-transferase protein fused to the C-terminus of the SARS protein. The recombinant proteins can be purified by single step column chromatography on either Nickel chelating Sepharose or Glutathione-Sepharose 4B resin. These procedures are very rapid and generally produce protein of 60-90% purity, which is suitable for raising specific antisera (Pizza et al. (2000) Science 287:1816-20). Five mice and two rabbits for each recombinant protein can be immunized SC with 20 and 50 μg recombinant protein, respectively, given in IFA as adjuvant, at day 0, 14 and 28. Sera are collected at day 7, 21 and 35 to assess specific titers before euthanasia of the animals for collection of blood and removal of spleens.

For the detection of impurities (e.g. Vero cell derived proteins) in the vaccine preparation, rabbit serum reactive against Vero-derived proteins can be used. Such antisera are obtained by immunizing rabbits with at least 10 μg of Vero cell lysate with CFA/IFA. The sera can be verified for reactivity against Vero-derived proteins in western blots. For more specific antisera against specific relevant cell-derived proteins that tend to be co-purified with the virus, mock-infected cell culture harvest that have undergone the purification process can be prepared and used for immunizing rabbits.

Methods to determine neutralization titers of sera from immunized animals and humans can be developed, without the constraints of using infectious SARS-CoV in a BSL-3+ laboratory. One such strategy will be to use recombinant antigens, particularly Spike protein or Spike-derived epitopes, and to develop ELISA assays for measuring antibodies against the target protein. Suitable epitopes allow a correlation to be established between the ELISA values and virus neutralization assay values. This approach provides a faster and more efficient (higher-throughput) comparison of specific and protective antibody titers. This ELISA test is also the ideal tool to monitor specific antibodies in safety trials, where several hundred animal sera must be tested.

Another strategy is to combine structural elements from both the pathogenic SARS-CoV and the non-pathogenic coronavirus mouse hepatitis virus (MHV) to construct chimeric virus-like particles (VLPs) that can be labeled. The assay is based on fusion between octadecyl rhodamine (R18)-labeled VLPs and cells (Hoekstra et al. (1984) Biochemistry 23:5675-81). The method relies on the relief of fluorescence self-quenching of R18 incorporated into VLPs upon fusion with cellular membranes. Coronavirus VLPs have been shown to mimic native virions with respect to their appearance in the electron microscope (EM) and their biological activities. As they do not contain viral RNA, however, then they cannot cause a productive infection (Vennema et al. (1996) EMBO J 15:2020-2028). The VLP system can be used for the mouse hepatitis virus (MHV) strain A59 (MHV-A59) )Godeke et al. (2000) J Virol 74:1566-15) containing a chimeric S protein. The protein chimera, consisting of the ectodomain of the SARS-CoV and the transmembrane and endodomain (64 C-terminal amino acid residues) from the MHV spike protein, can be co-expressed with the MHV M (membrane) and E (envelope) protein in OST-7 cells )Godeke et al.). VLPs secreted in the supernatant are harvested, purified and labeled with octadecyl rhodamine (R18) (Hoekstra et al). A constant amount of VLPs is incubated with a serial dilution of sera at 37° C. for 1 hour in a 96-well plate. Subsequently, cells expressing the receptor for the SARS-CoV, the angiotensin-converting enzyme 2 (ACE2) (Li et al. (2003) Nature 426:450-54) is be added and the extent of fusion can be measured with a fluorescence spectrophotometer.

A final strategy to monitor the ability of sera to inhibit cell-cell fusion interactions between cells expressing the SARS-CoV S protein and a human cell line expressing the angiotensin-converting enzyme 2 (ACE2), a functional receptor for SARS-CoV (Li et al.). This reporter gene-based assay uses the fluorescent shift (green to blue) of the fluorogenic substrate CCF2/AM (AM=acetoxymethyl) upon cleavage by β-lactamase (Bla) as read-out for cell-cell fusion (Zlokarnik et al. (1998) Science 279:84-88). For this assay, a BHK-derived cell line, stably expressing Bla and the SARS-CoV S protein is generated. In addition, a human cell line expressing ACE2 on its surface is used. BHK cells, expressing the S protein on their surface and Bla in their cytosol are incubated with serial dilutions of the sera to be tested for 1 h at 37° C. The cell line expressing the ACE2 is loaded with 1 μM CCF2/AM for 1 h at 22° C., washed twice with PBS, and co-cultivated with the BHK cells. In case of cell-cell fusion, Bla cleaves the substrate, resulting in a green blue shift with excitation at 409 nm. Inhibition of fusion by sera thus provides a detectable change.

### Example 16

### Stabilisation of Inactivated SARS-CoV

Although the purified inactivated SARS-CoV vaccine is capable of inducing potent neutralizing antibody responses in animals, it is relatively instable and can benefit from formulation to increase stability for an acceptable period of time. Suitable formulation changes include the use of various buffer systems, pH ranges, stabilizing excipients (e.g. sugars and sugar alcohols, amino acids, etc.) etc.. Stability testing can be conducted in real-time at normal storage temperatures, or can be conducted in an accelerated manner by using elevated temperatures. Vaccine stability can thus be increased to approximately one year or longer. Lyophilized vaccine formulation can also be used to extend shelf-life, possibly with further additives for stability during lyophilisation.

### Example 17

### Dose and Schedule Optimization for Inactivated Virus

Animal models of SARS-CoV infection have been reported, including mice, ferrets and macaques. As mentioned in example 4 above, mice immunized with the BPL-SARS-CoV vaccine achieve neutralizing antibody titers in the range of 1:100-1:1000, similar to levels found in convalescent patients, and are 100% protected from infection with a challenge virus. While the mouse challenge model is limited only to infection but not disease, ferrets and macaques are useful models of the human SARS disease. Two to four days after inoculation with SARS-CoV, both ferrets and macaques have been found to shed infectious SARS-CoV particles from the throat, nose and pharynx, as demonstrated by RT-PCR and/or virus isolation on Vero cells. At approximately the same time, the infected animals became lethargic, show respiratory distress and eventually die. Histologically, SARS-CoV infection in these animals associates with pulmonary lesions of different severity, similar to those found in biopsied lung tissue and autopsy material from SARS patients. With the availability of these models, preclinical studies with vaccines can be performed initially in mice for immunogenicity readouts, while efficacy of optimal doses and schedules can be assessed in the ferret and macaque models.

Initial studies in mice are used to determine the optimal dose and schedule required to elicit the highest levels of neutralizing antibody, with titers at least in the range of 1/100- 1/1000. In parallel to the assessment of neutralizing activity, other features of the humoral immune response and cellular immune responses can be investigated. In particular sera from immunized mice can be assessed for the isotype (IgG1 vs. IgG2a) of the Spike-specific antibody response. Also, the frequencies of splenic CD4+ T cells producing IFN-γ and IL-4 in response to BPL-SARS-CoV particles will be assessed by ELISPOT and ELISA. These experiments can provide insight into the quality of the T cell response helping the priming of a protective antibody response.

Increasing vaccine doses can be tested (e.g. from 5 to 20 μg of BPL-SARS-CoV alone or mixed with an equal volume of MF59-citrate), administered SC to anesthetized mice in 100 μl of inoculum. Groups of BALB/c mice, 10 per treatment, are immunized, with priming at day 0 and boosting at days 14 and 28. Secondary endpoints compare the kinetics of neutralizing vs. Spike-specific antibody titers and assess the Th1/Th2 profile of the specific immune response, and so neutralizing and Spike-specific antibody titers are assessed at days 7, 21, 35, and at 2, 3, 4, and 5 months after priming. The IgG2a and IgG1 titers of Spike-specific antibodies are determined at days 21, 35, and at 2, 3, 4, and 5 months after priming. Proliferation and IFN-γ and IL-4 production by splenic T cells against recombinant Spike protein from SARS-CoV are assessed at day 42, and at the end of the 5th month. Peripheral blood is collected at days 7, 21, 35, and at 2, 3, 4, and 5 months after priming. Spleen cells will be obtained at day 42 and at the end of the 5th month. Neutralizing and Spike-specific antibody titers and isotypes are determined by inhibition of infection of Vero cells and by ELISA, respectively. Proliferation of splenic cells is determined by ³[H]-thymidine uptake. Frequencies of splenic IFN-γ and IL-4 producing CD4⁺ T lymphocytes is determined by ELISPOT and FACS analysis.

Based on mouse results, the BPL-SARS-CoV vaccine can be tested in ferrets for the induction of protective neutralizing antibody titers. Ferrets are immunized according to a similar schedule as the mice and at the dose that elicits the highest neutralizing antibody titers in mice at day 35 after the second boost. Three groups of ferrets, 6 per treatment, are immunized with BPL-SARS-CoV, alone or mixed with an equal volume of MF59-citrate, administered SC to anesthetized animals in 200 μl of inoculum. The animals are primed at day 0 and boosted at days 14 and 28. Peripheral blood is collected at days 7, 21, and 35. Neutralizing and Spike-specific antibodies titers are determined by inhibition of SARS-CoV infection of Vero cells and by ELISA, respectively. Each group of ferrets is used to assess efficacy of the BPL-SARS-CoV in protecting vaccinated animals from infection and/or disease. Anesthetized animals are challenged intratracheally, two weeks after the last boost, with 10⁶ median tissue culture infectious dose units (TCID₅₀) of the SARS-CoV CDC strain. Infection by SARS-CoV can be assessed by taking nasal, pharyngeal and rectal swabs from animals for 20 days after challenge (Martina et al. supra). The presence of SARS-CoV in sample materials can be assessed by RT-PCR and infection assay of Vero cells. Animals can be monitored for clinical signs of SARS disease by assessing sleeping time, temperature, respiratory symptoms, diarrhea, body weight and survival. Protection can be determined by the magnitude and duration of virus shedding, by duration and severity of disease symptoms, and by percentage of surviving animals. The formulation eliciting the highest neutralizing antibody titers at day 35 can then be tested against a two-fold higher dose of BPL-SARS-CoV given in the same formulation in the same regimen.

Additional studies can evaluate immunogenicity and efficacy of the candidate vaccine in non-human primates. Three groups of adult cynomolgus macaques, 4 per treatment, are immunized with BPL-SARS-CoV, alone or mixed with an equal volume of MF59-citrate, administered SC to anesthetized animals in 500 μl of inoculum. The BPL-SARS-CoV vaccine can be tested at the dose eliciting the highest neutralizing antibody titers in ferrets at day 35 after the second boost. The animals are primed at day 0 and boosted at 3 and 6 weeks. Peripheral blood is collected at weeks 1, 4, and 7. A secondary endpoint is to assess the Th1/Th2 profile of the specific immune response. Neutralizing and Spike-specific antibody titers and frequencies of peripheral blood CD4+ T cells producing IFN-γ and IL-4 in response to the recombinant SARS-CoV Spike protein is thus assessed at weeks 1, 4, and 7. Neutralizing and Spike-specific antibody titers can be determined by inhibition of SARS-CoV infection of Vero cells and by ELISA, respectively. Intracellular cytokine staining and FACS analysis will be used to quantify IFN-γ- and IL-4-producing CD4⁺ T cells. The macaques can also be used to assess efficacy of the BPL-SARS-CoV in protecting vaccinated animals from infection and/or disease. Anesthetized macaques can be challenged two weeks after the last boost with 10⁶ median tissue culture infectious dose unit (TCID₅₀) of the SARS-CoV CDC strain in a 5 ml volume. A few drops of the virus can also be administered on each of the conjunctiva, 0.5 ml in the nose and the remainder in the trachea. Infection by SARS-CoV can be assessed by taking nasal, pharyngeal, and rectal swabs, and feces from animals for 20 days after challenge (Fouchier et al. (20030 Nature 423:240). The presence of SARS-CoV in sample materials can be assessed by RT-PCR and infection assay of Vero cells. Animals can also be monitored for clinical signs of SARS disease by assessing sleeping time, temperature, respiratory symptoms, diarrhea, body weight and survival. Protection can be determined by the magnitude and duration of virus shedding, by duration and severity of disease symptoms, and by percentage of surviving animals.

Mice

| Group | Treatment | Dose/Route | Sampling interval | Number of mice |
|---|---|---|---|---|
| 1-3 | BPL-SARS-CoV | 20, 10, 5 μg/SC | 7, 21, 35 d; | 10 per dose level |
| | | | 2, 3, 4, 5 m; | |
| 4-6 | BPL-SARS-CoV | 20, 10, 5 μg/SC | 42 d | 10 per dose level |
| 7-9 | BPL-SARS-CoV MF59 | 20, 10, 5 μg/SC | 7, 21, 35 d; | 10 per dose level |
| | | | 2, 3, 4, 5 m; | |
| 10-12 | BPL-SARS-CoV MF59 | 20, 10, 5 μg/SC | 42 d | 10 per dose level |
| 13 | MF59 | NA/SC | 7, 21, 35 d; | 10 + 10 (sacrificed at |
| | | | 2, 3, 4, 5 m; | 42 d and end 5 m) |
| 14 | Saline | NA/SC | 7, 21, 35 d; | 10 + 10 (sacrificed at |
| | | | 2, 3, 4, 5 m; | 42 d and end 5 m) |

Ferrets

| | | | Sampling | |
|---|---|---|---|---|
| Group | Treatment | Route | interval | No. of ferrets |
| 1 | BPL-SARS-CoV | SC | 7, 21, 35 d | 6 |
| 2 | BPL-SARS-CoV-MF59 | SC | 7, 21, 35 d | 6 |
| 3 | Saline | SC | 7, 21, 35 d | 6 |

Macaques

| | | | | No. of |
|---|---|---|---|---|
| Group | Treatment | Route | Sampling interval | macaques |
| 1 | BPL-SARS-CoV | SC | 1, 4, 7 w | 4 |
| 2 | BPL-SARS-CoV - | SC | 1, 4, 7 w | 4 |
| | MF59 | | | |
| 3 | Saline | SC | 1, 4, 7 w | 4 |

### Example 18

### Human T Cell Responses

Asia prelude to initiation of clinical studies in humans, the reactivity of peripheral blood T lymphocytes from healthy donors with different HLA haplotypes can be assessed using the in vitro priming technique (Abrignani et al. (1990) Proc Natl Acad Sci USA 87:6136-40). The aim of this study is to have a first indication of the immune-dominant T cell epitopes in SARS-CoV proteins. Briefly PBMCs from 20 healthy donors with different HLA haplotypes will be cultured in medium containing 5% autologous serum, in the presence of different concentration of SARS-BPL-CoV particles in the range from 0.5 to 20 μg/ml. The expression of activation markers will be assessed after 24 and 48 hours. Frequencies of IFN-γ- and IL-4-producing T lymphocytes will be assessed after 12 h and after 15 days in culture, in the presence of 100 U/ml recombinant human IL-2. Activated and cytokines producing CD4 T lymphocytes will be sorted and eventually cloned as single cells using FACS technologies. The CD4+ T cell repertoire from human subjects with different HLA will be assessed by proliferation assays of the CD4+ T cell lines and clones against autologous EBV-transformed cell lines loaded with 15-mer overlapping peptides from the most relevant structural and non structural protein of the SARS-CoV.

When moving to actual human trials, safety and immune responses will be evaluated in healthy adults following intramuscular immunization with escalating doses of the BPL-inactivated SARS-CoV vaccine, with MF59 adjuvant being included or omitted depending on preclinical data. Three/four immunizations will be given at 0, 1, 6 months in the first cohort, and at 0, 1, 2, 6 months and 0, 2, 6 weeks in the second and third cohorts respectively. The trial will be observer blind and placebo controlled. Subjects will be randomized into each dose level. Immune response parameters to be measured will include serum neutralizing antibodies, ELISA antibodies and peripheral blood IFN-gamma-producing CD4+ T cells by intracellular cytokine staining.

| | Antigen | Administration | No. treated | No. subjects | Sampling |
|---|---|---|---|---|---|
| Group | dose (μg) | schedule | subjects | with placebo | interval |
| A1 | 10 | 0, 1, 6 months | 18 | 6 | 0, 1, 2, 6, 7 mos |
| A2 | 20 | 0, 1, 6 months | 18 | 6 | 0, 1, 2, 6, 7 mos |
| B1 | 10 | 0, 1, 2, 6 months | 18 | 12 | 0, 1, 2, 6, 7 mos |
| B2 | 20 | 0, 1, 2, 6 months | 18 | 12 | 0, 1, 2, 6, 7 mos |
| C1 | 10 | 0, 2, 6 weeks | 18 | 12 | 0, 2, 6, 10, 30 wks |
| C2 | 20 | 0, 2, 6 weeks | 18 | 12 | 0, 2, 6, 10, 30 wks |

### Example 19

### Selection of CHO Cell Lines for Spike Protein Expression

Methods for the derivation of Chinese Hamster Ovary (CHO) cell lines that stably express viral envelope glycoproteins that are conformationally intact, appropriately glycosylated and efficiently bind neutralizing antibodies are well established for HIV and HCV (Srivastava et al. (2002) J Virol 76:2835-47; Srivastava et al. (2003) J Virol 77:11244-259; Heile et al. (2000) J Virol 74:6885-92). The same techniques can be applied to SARS-CoV, to generate two different stable CHOK-1 cell lines producing either full-length or truncated SARS Spike proteins. The Spike proteins can be expressed using the constructs described herein, but without the hexa-His tags. These proteins can compared for their ability to produce neutralizing antibodies in immunized animals as well as for their expression levels in CHOK-1 cells.

A pCMV3 vector expressing Spike can be used for the derivation of stable CHOK-1 cell lines, containing the CMV enhancer/promoter, ampicillin resistance, and a fused DHFR and attenuated neomycin gene for selection purposes. Stable cell lines can produced using the neomycin selection system in CHOK-1 cells. Clones can be sequenced to verify the integrity of the insert, and transient transfections can be performed using Trans-LT1 polyamine transfection reagent (PanVera Corp., Madison, Wis.) to assess the expression level and also the integrity of the expressed protein by ELISA and western blot analysis.

Initial CHO cells will be selected to be free from TSE/BSE contaminants and risks according to relevant regulatory standards. To construct cell lines, procedures involve transfection, primary screening with selective medium, followed by subcloning to assure purity of cell lines. Cell supernatants can be assayed using an antigen capture ELISA to quantify expression levels at all stages of selection and amplification. For full-length Spike expression, methanol fixed cells can be screened for internal expression by immunofluorescent staining using a rabbit anti-SARS antibody. Successive measurements at the T75-flask stage of expansion canbe employed to assure stability of expression levels. The molecular mass and integrity of the expressed proteins can be checked by PAGE both under native and reducing and denaturing conditions, followed by immunoprobing.

The pCMV3 vectors expressing SARS-CoV Spike proteins in either full-length or truncated forms can be introduced into CHOK-1 cells using the Trans-LT-1 reagent and non-selective media. 24-48 hours post-transfection, depending on cell density, cells are split at a 1:5 ratio and the medium can be changed to selective media containing neomycin at 500 μg/ml. Any bovine serum used in these procedures will be from TSE-free sources that meet regulatory standards. Ten to fourteen days later, individual colonies can be picked and transferred to 96 well plates and cultured in complete non-selective medium. When approximately 80% of the wells are confluent, 24 hour supernatants can be screened by Spike capture ELISA. For initial expression of full length Spike protein, cells can be fixed with methanol and screened by immunofluorescent staining using a rabbit anti-SARS antibody. After low-expressing cell lines have been eliminated and there are fewer than 20-30 cell lines, capture ELISA and western blots can then be used to determine the expression level after cell lysis. A portion of each cell line can be pelleted, weighed and lysed in 1% Triton lysis buffer for determination of expression levels. Three to four clones producing the highest levels of spike protein in correct structure and conformation can be expanded to three-liter bioreactors and adapted to low serum suspension culture conditions for scale-up.

The antigen capture ELISA assay for the SARS spike protein can be performed using 96 well flat-bottom plates coated with 250 ng per well of purified immunoglobulin obtained from rabbit sera that were immunized with inactivated SARS virus. Supernatant or lysate samples are added and incubated for 2 hours at 37° C. Bound antigen is reacted against pooled SARS^{+ve} serum or high affinity monoclonal antibody either human or mouse against SARS spike protein and detected using appropriate species-specific peroxidase-conjugated second antibody. The plates are developed using TMB substrate (Pierce, Rockford, Ill.), read at a wavelength of 450 nm, and the concentration of protein per ml sample is derived from a standard curve (OD vs. protein concentration) based on serial dilutions of a known concentration of recombinant spike protein.

The immunoprobing analysis will also be performed following the standard methods described by Srivastava et al. (2002) supra. Briefly, 10-20 μl of the sample is analyzed on 4-20% SDS PAGE under non-reducing/denaturing conditions with mild heating. The proteins are then transferred onto nitrocellulose membranes and reacted against polyclonal anti-Spike rabbit serum, followed by anti-rabbit Ig conjugated to Alexa 688 (Molecular Probes, Oreg.). The blots are scanned using an infrared imaging system.

The highest expressing candidate cell lines will be screened for Spike protein expression and stability in small-scale (3 liter) perfusion bioreactors. The candidate clones will be further evaluated for level of expression as well as integrity of expressed protein, and subsequently tested for expression stability in the absence of selection. The selected clones also will be tested for maintenance of the DNA sequence integrity of the integrated SARS spike protein gene. To quickly monitor the expression levels in small flasks and in the three liter evaluation cultures, a lectin-based process (Gluvanthus Nivalis lectin) has been developed to isolate SARS spike protein to a degree of purity that allows semi-quantitation and characterization of the protein in CHO supernatant. Full-length Spike protein will be obtained from Triton X-100 detergent extracted cells and then captured on GNA lectin, followed by hydroxyapatite and SP chromatograph. Eluted protein is then characterized by: (1) polyacrylamide gel electrophoresis (PAGE) and Coomassie staining, (2) immunoprobing with anti-SARS rabbit sera, (3) structural characterization using size exclusion chromatography (SEC), as well as mass spec analysis using MALDI-TOF.

Productivity from the CHO cell line expressing SARS spike protein should be at least 2 mg/L and for full-length Spike protein will be 3 mg/100 gm of cells, at steady-state cell density. Yield from one 45 day, 2.5-liter bioreactor will be ˜1000 mg crude protein.

### Example 20

### Purification of Spike Protein for Human Vaccines

To purify SARS spike protein for the purpose of producing GMP grade material for human use, the following basic process is used, with all steps being performed at 2-8° C.: the starting material, concentrated CHO cell culture supernatant (20-30×) is thawed and filtered through a 0.45 μm membrane; this material is heavily contaminated proteins from culture, as well as DNA; the first purification step is affinity chromatography using Gluvanthus Nivalis (GNA), a lectin that preferentially recognizes terminal mannose containing carbohydrates; glycosylated proteins, including SARS spike protein are captured and non-glycosylated proteins, as well as DNA, do not bind to this column; the GNA column is followed by two chromatographic steps operated in the flow through mode; the anion exchanger, DEAE, and ceramic hydroxyapatite (cHAP); DEAE binds some contaminating supernatant proteins and DNA, whereas cHAP binds any contaminating serum proteins; full-length Spike protein is purified from the cell pellet; the cells are lysed with Triton X-100 and full-length Spike protein is then captured on GNA lectin, followed by hydroxyapatite and SP chromatography.

The purified SARS spike can be further treated to remove adventitious viruses: viral inactivation at pH 3.5 for 1 hour; the sample is then concentrated and diafiltered into a buffer at pH 4 and finally captured the purified protein using SP resin; the spike protein binds to this resin and many viruses flow through.

The spike protein is eluted, concentrated and diafiltered into formulation buffer. This formulated bulk product is then filtered through a DV50 viral removal membrane followed by filtration through a 0.2 μm membrane. The formulated bulk is filled into suitable containers e.g. into 3.0 ml vials, in a class 100 laminar flow hood.

In process testing at each step of the purification includes protein concentration, endotoxin (LAL), bioburden, and recovery.

Prior to human administration, a test for potency will evaluate the specific ability of the vaccine in an in vitro or in vivo test to effect a given response. The in vivo immunogenicity will be determined by dosing groups of 10 mice with various doses of the protein antigen. Sera will be analyzed for the presence of IgG antibodies using an ELISA. The criterion for passing will be based upon the number of vaccine treated animals that are seropositive compared to a reference standard. Other tests include General Safety, sterility, purity, identity of the vaccine (using an ELISA specific for Spike protein), and quantity & protein concentration (UV spectrophotometric absorbance procedure based on the molar absorbance of the aromatic amino acids).

Stability testing will be performed on the bulk drug substance and on the final container product. Bulk product will be evaluated at temperatures of −60° C. (recommended storage condition), 25±2° C. and 40±2° C. protected from light, at time points of 0, 3, 6, 9, 12 months. Final container product will be tested at temperatures of −60° C., and inverted at 5±3° C., 25±2° C., and 40±2° C. at time points of 0, 3, 6, 9, 12 months. Stability-indicating assays may include appearance, pH, protein content, SDS-PAGE, size exclusion HPLC, and container/closure integrity, performed on single samples of bulk and triplicate vials of final container material.

The protein purified in this way can be evaluated in mice, rabbits and ferrets as described in, and based on the results of, examples 4, 5, 8 and 9 above.

Initial experiments will be performed in mice to determine optimal dose and schedule of the GMP Spike protein required to elicit the highest levels of neutralizing antibody, with titers at least in the range of 1/100- 1/1000. Spike protein will be tested in the range from 5 to 40 μg, alone or mixed with an equal volume of MF59-citrate, to anesthetized mice in 100 μl of inoculum. Groups of BALB/c mice, 10 per treatment, will be immunized. The animals will be primed at day 0 and boosted at days 14 and 28. Secondary endpoints will be to compare the kinetics of neutralizing vs. Spike-specific antibody titers and to assess the Th1/Th2 profile of the specific immune response. Neutralizing and Spike-specific antibody titers will be assessed at days 7, 21, and 35 and at 2, 3, 4, and 5 months after priming; the IgG2a and IgG1 titers of Spike-specific antibodies will be determined at days 21 and 35, and at 2, 3, 4, and 5 months after priming; proliferation and IFN-γ and IL-4 production by splenic T cell against the recombinant Spike protein from SARS-CoV will be assessed at day 42 and at the end of the 5th month. Peripheral blood will be collected at days 7, 21, and 35 and at 2, 3, 4, and 5 months after priming; spleen cells at day 42 and at the end of the 5th month. Neutralizing and Spike-specific antibody titers and isotypes will be determined by inhibition of SARS-CoV infection of Vero cells and by ELISA, respectively. Proliferation of splenic cells will be determined by ³[H]-thymidine uptake. Frequencies of splenic IFN-γ and IL-4 producing CD4+ T lymphocytes, will be determined by ELISPOT and FACS analysis.

Next, the optimal dosing and schedule for recombinant Spike vaccine will be determined in ferrets. Based on the mouse results, the Spike vaccine eliciting the highest antibody neutralizing titers will be tested against a two-fold higher dose of recombinant Spike protein given in the same formulation. Three groups of ferrets, 6 per treatment, will be immunized SC under anesthesia with 200 μl of inoculum. The animals will be primed at day 0 and boosted at days 14 and 28. Peripheral blood will be collected at days 7, 21, and 35. Neutralizing and Spike-specific antibodies titers will be determined by inhibition of SARS-CoV infection of Vero cells and by ELISA, respectively. Similar to the previous ferret studies, each group of animals will be used to assess efficacy of the vaccine in protecting immunized animals from infection and/or disease.

Immunogenicity and efficacy of the candidate vaccine also will be evaluated in nonhuman primates. Three groups of adult cynomolgus macaques, 4 per treatment, will be immunized with recombinant SARS-CoV Spike protein, alone or mixed with an equal volume of MF59-citrate, administered SC to anesthetized animals in 500 μl of inoculum. The Spike protein vaccine will be tested at the dose eliciting the highest neutralizing antibody titers in ferrets at day 35. The animals will be primed at day 0 and boosted at 3 and 6 weeks. Peripheral blood will be collected at weeks 1, 4, and 7. A secondary endpoint will be to assess the Th1/Th2 profile of the specific immune response, as described above (neutralizing and Spike-specific antibody titers, frequencies of peripheral blood CD4+ T cells producing IFN-γ and IL-4 in response to the recombinant Spike protein, assessed at at weeks 1, 4, and 7).

Finally, human phase I, placebo-controlled, dose-escalation, safety/immunogenicity trials will be performed for the IM recombinant SARS vaccine with MF59 adjuvant. The trial will evaluate safety and immune responses in healthy adults following immunization with escalating doses of SARS recombinant vaccine with MF59 adjuvant, administered intramuscularly. Three/four immunizations will be given at 0, 1, 6 months. The trial will be observer blind and placebo controlled. Subjects will be randomized into each dose level. Immune response parameters to be measured include serum neutralizing antibodies, ELISA antibodies and peripheral blood IFN-γ-producing CD4+ T cells by intracellular cytokine staining:

| | Vaccine | | | No. of | |
|---|---|---|---|---|---|
| | Antigen | | No. of | subjects | |
| | dose | Administration | treated | with placebo | Sampling |
| Group | (μg) | schedule | subjects | (MF59) | interval |
| A1 | 50 | 0, 1, 6 months | 18 | 6 | 0, 1, 2, 6, 7 |
| | | | | | months |
| A2 | 100 | 0, 1, 6 months | 18 | 6 | 0, 1, 2, 6, 7 |
| | | | | | months |

### Example 21

### Comparison of Inactivated Virus and Purified Spike Protein

Immunogenicity and efficacy of the inactivated virus vaccine and the purified Spike protein can be compared in non-human primates. Three groups of adult cynomolgus macaques, 4 for treatment, will be immunized with recombinant SARS-CoV Spike protein from CHO cell lines or with BPL-SARS-COV, given in the dose and formulation eliciting the highest neutralizing antibody titers in previous immunogenicity challenge experiments, administered SC to anesthetized animals in 500 μl of inoculum. The animals will be primed at day 0 and boosted at 3 and 6 weeks. Peripheral blood will be collected at weeks 1, 4, 7. A secondary endpoint will be to assess the Th1/Th2 profile of the specific immune response, as described above.

| | | | Sampling | No. of |
|---|---|---|---|---|
| Group | Treatment | Dose/Route | interval | macaques |
| 1 | Rec-Spike protein + or − | Y μg/SC | 1, 4, 7 w | 4 |
| | MF59 | | | |
| 2 | BPL-SARS-CoV + or − | Y μg/SC | 1, 4, 7 w | 4 |
| | MF59 | | | |
| 3 | Saline | NA/SC | 1, 4, 7 w | 4 |

### Example 22

### Expression in Yeast

Yeast is a useful and inexpensive eukaryotic expression system. Yeast-expressed proteins are used in recombinant hepatitis B virus vaccines, and recombinant SARS antigens may also be expressed in yeast for vaccine purposes. Yeast-expression is also convenient for the production of antigens for preparing monoclonal and polyclonal antitobodies, or for use in serological assays.

The nucleocapsid protein (N) and two different versions of the spike glycoprotein (S) from SARS coronavirus FRA strain (AY310120) were cloned for expression in S. cerevisiae:

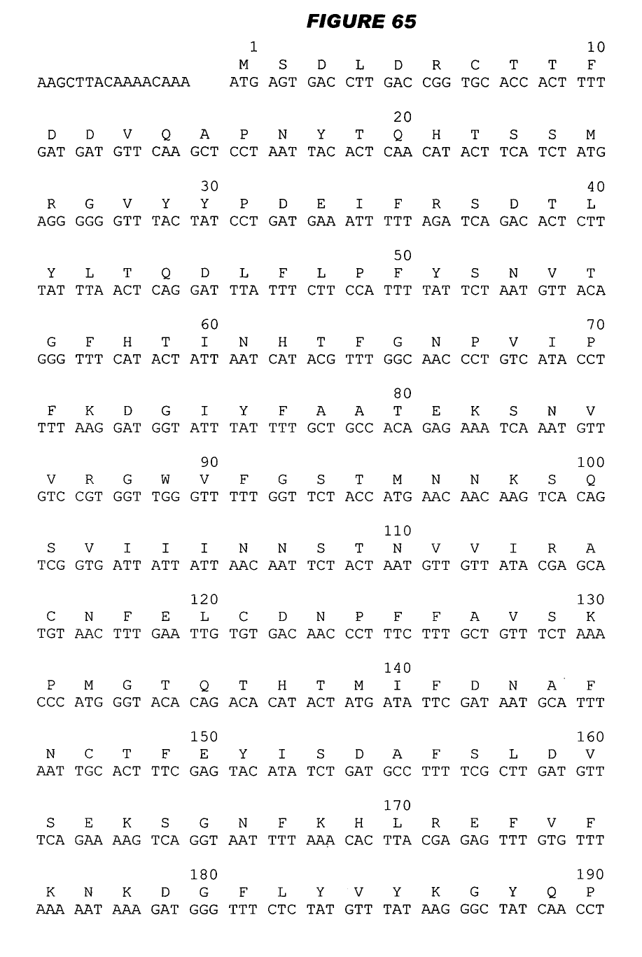

SARS N: aa 1-422 (coordinates 28120-29388 of AY310120 strain)—FIG. 65

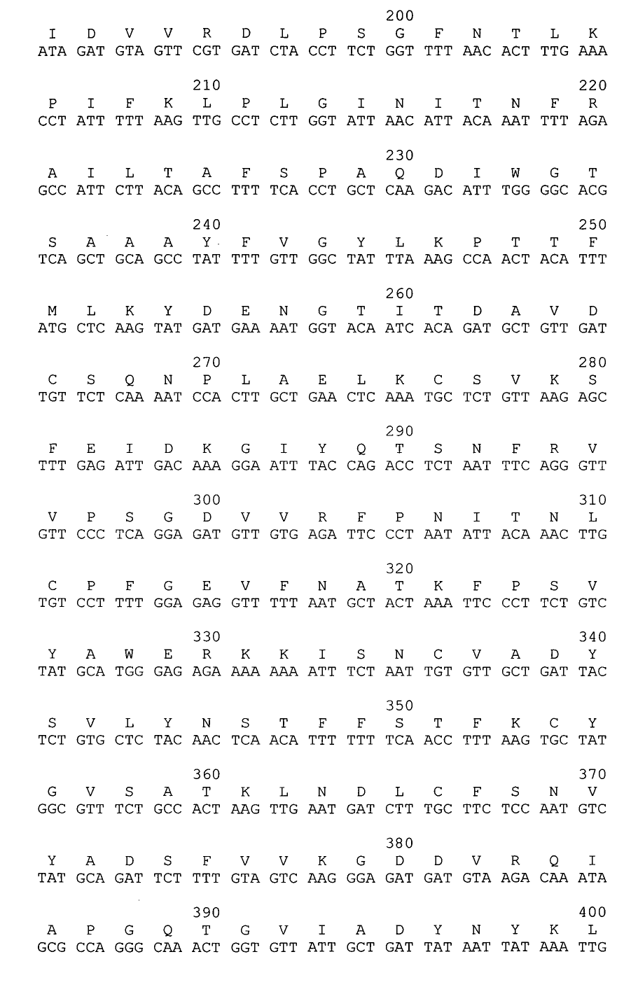

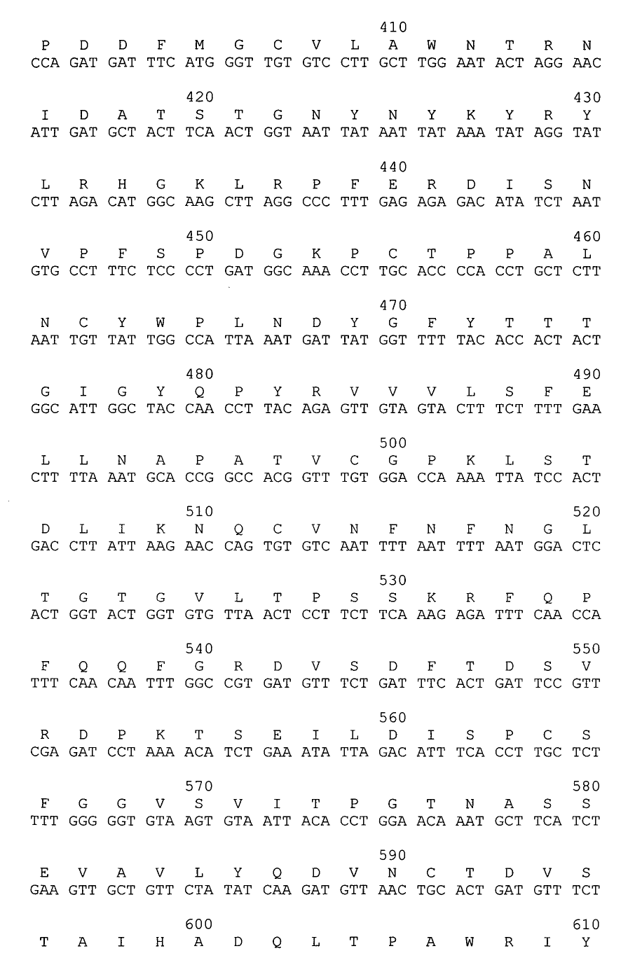

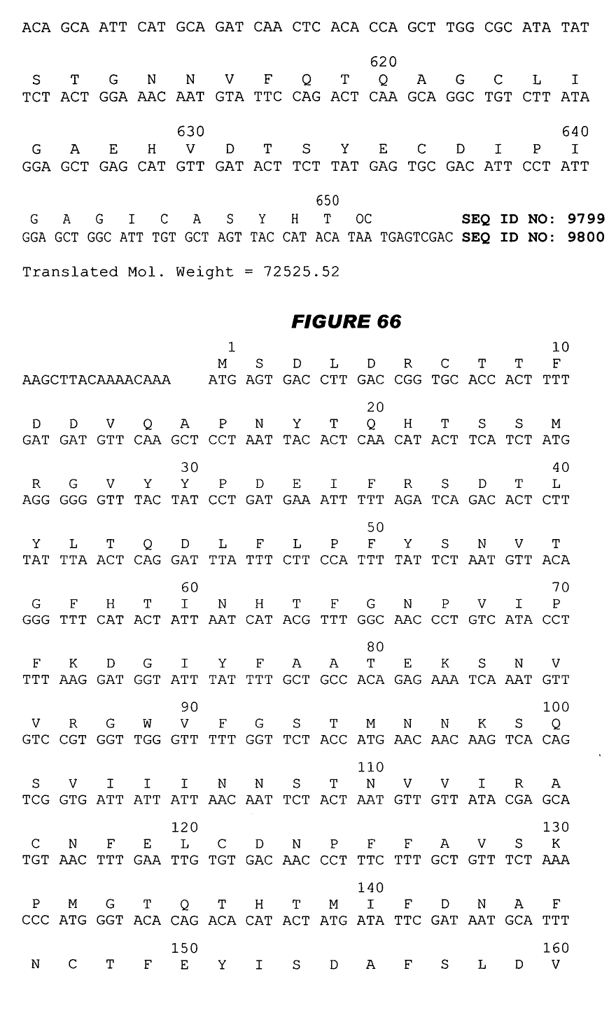

SARS spike: aa 14-1195 (transmembrane domain and cytoplasmic tail deleted)—FIG. 66

SARS spike: aa 14-662 (S1 domain)

To make the S1 construct, a XhoI-NotI fragment of approximately 3733 bp encoding the full-length spike glycoprotein was the starting point. PCR was used to amplify the full-length gene in two pieces: XbaI-BlnI of 2440 bp and BlnI-SalI of 1306 bp. These fragments were subcloned into commercial vectors (Novagen): pT7Blue2 XbaI-BlnI (5′ end of spike glycoprotein) and pT7Blue2 BinI-SalI (3′ end of spike glycoprotein; FIG. 58), respectively. The following primers were used in the subsequent PCR reactions: Spk-1 (5′) SEQ ID NO: 9785; Spk-2 (5′) SEQ ID NO: 9786; Spk-3 (5′) SEQ ID NO: 9787; Spk-4 (5′) SEQ ID NO: 9788.

E. coli HBO101 competent cells were transformed with the PCR ligation product and plated on Luria agar plates, containing 100 μg/ml ampicillin. The desired clones were identified using miniscreen DNA analysis. After sequence verification and plasmid amplification of the desired subclones, it was desirable to eliminate the internal SalI site present in the XbaI-BlnI portion of the spike sequence in order to facilitate future cloning into the yeast expression vector (BamHI-SalI). Therefore, we prepared a CelII-MfeI vector from the pT7Blue2 XbaI-BlnI (5′ end Spike) subclone to eliminated a 143 bp sequence containing the SalI site. Kinased oligos DSI-6 (SEQ ID NOS: 9789-9794) were then ligated into the CelII-MfeI vector to replace the 143 bp that were removed to mutate the SalI site (no aa changes), creating pT7Blue2.XbaI-BlnIΔsal.

The 5′ XbaI-BlnI (from pT7Blue2.XbaI-BlnI ΔSal) and the 3′ BlnI-SalI (from pT7Blue2 BlnI-SalI) spike glycoprotein inserts were gel-purified and ligated them into the p893-1 XbaI-SalI vector (a vector derived from pLitmus 38 (New England Biolabs) with the alpha-factor leader sequence cloned into the BamHI-SalI sites of the MCS). The resulting full-length SARS Spike coding sequence was named p893-1.SARS Spike 1255 #9 (FIG. 58).

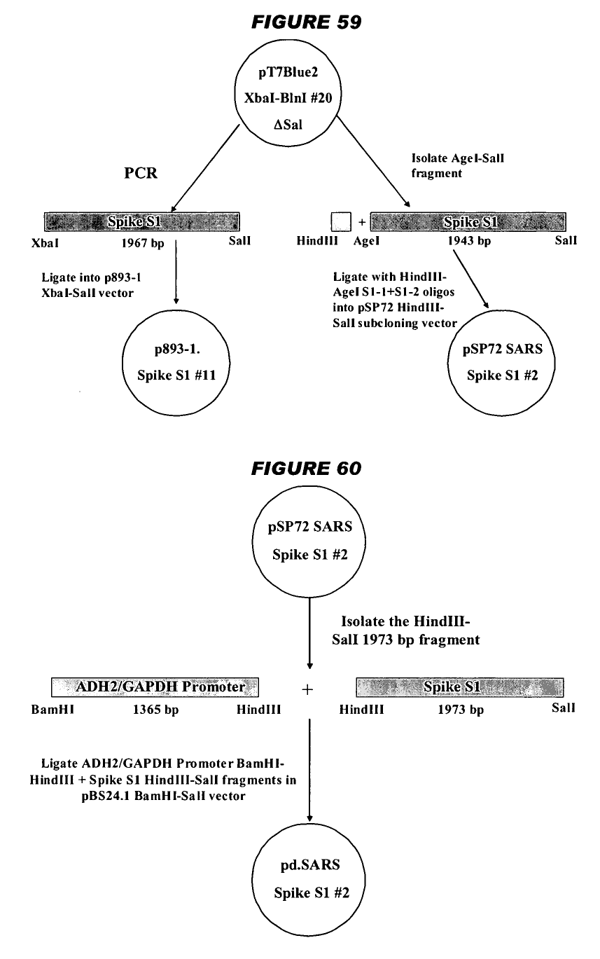

E. coli HB 101 competent cells were transformed with the oligo replacement ligation product and plated on Luria agar plates, containing 100 μg/ml ampicillin. The desired clones were identified using miniscreen DNA analysis. After sequence verification of the positive clones, pT7Blue2 Xba-Bln ΔSal was chosen for use as a template for PCR reactions to amplify the Spike S1 1967 bp Xba-Sal fragment. The fragment was then subcloned into the p893-1 Xba-Sal vector, sequence verified, and named it p893-1.Spike S1 #11 (FIG. 59).

In order to clone into the S.cerevisiae expression vector, pBS24.1, the 5′ end of the S1 sequence had to be modified from XbaI to HindIII to allow ligation with the 3′ HindIII end of the ADH2/GAPDH BamHI-HindIII promoter fragment. From pT7Blue2 Xba-BlnΔSal (described above) an AgeI-SaII 1943 bp fragment was gel-purified. This fragment was ligated along with a synthetic pair of HindIII-AgeI 30 bp kinased oligos (S1-1+S1-2 creating the necessary 5′ HindIII site) into the pSP72 HindIII-SalI commercial subcloning vector (named pSP72.SARS Spike S1 #2; FIG. 59). S1-1 had SEQ ID NO: 9795 and S1-2 has SEQ ID NO: 9796.

After sequence verification of the positive clone from miniscreen DNA analysis, the HindIII-SalI fragment was gel purified. The 1365 bp BamHI-HindIII ADH2/GAPDH promoter fragment was ligated along with the 1973 bp HindIII-SalI S1 fragment into the pBS24.1 BamHI-SalI vector creating the genetically engineered pd.SARS Spike S1 #2 expression plasmid (FIG. 60).

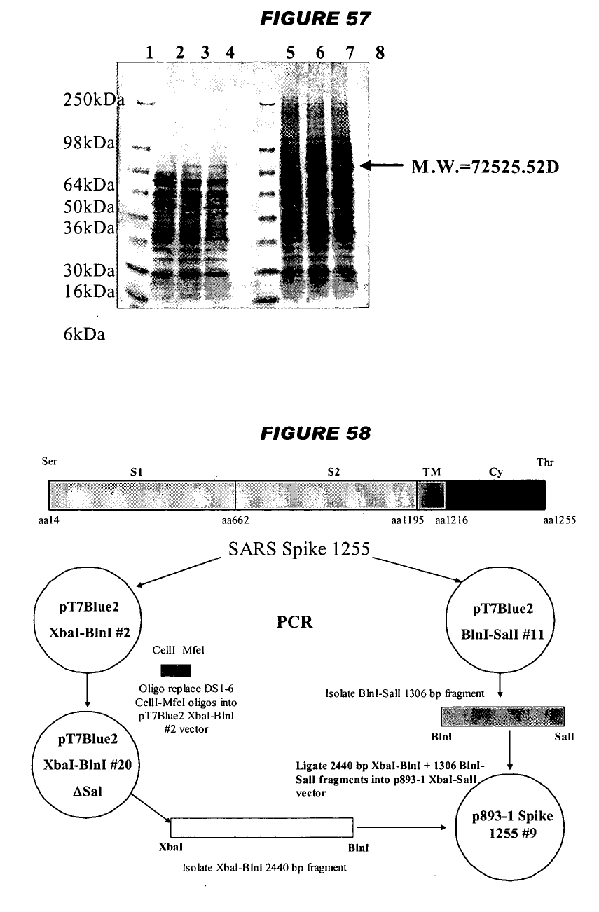

S. cerevisiae strain AD3 was transformed with pd.SARS Spike S1 #2 and single transformants were checked for expression after depletion of glucose in the medium. The recombinant protein was expressed at high levels in yeast, as detected by Coomassie blue staining. In particular, yeast cells were transformed with the SARS S1 expression plasmid using the Invitrogen S.c. EasyComp™ Transformation Kit. Expression in shown in FIG. 57.

To express Spike 1195 protein, which does not contain the trans-membrane (TM) region or cytoplasmic tail that are present in the full-length SARS construct, the following series of genetic manipulations was performed:

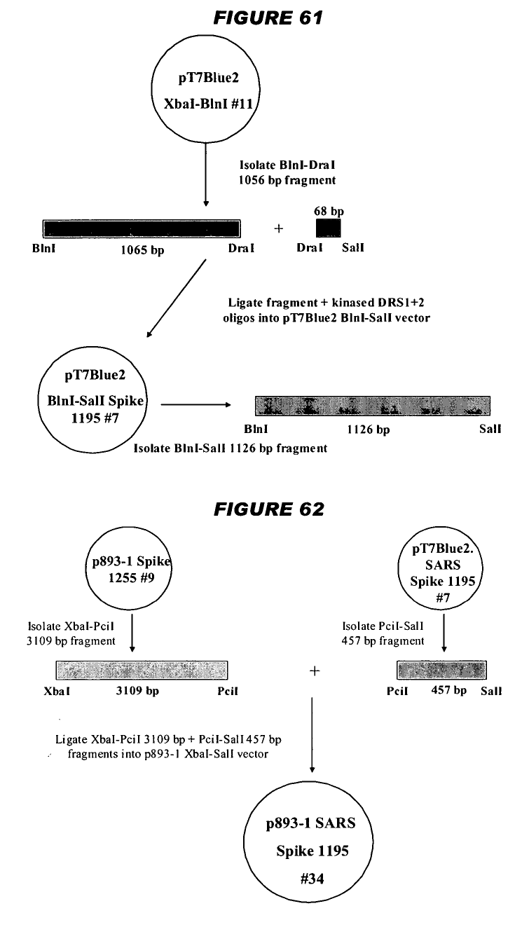

From pT7Blue2 BlnI-SalI #11 (described above) a BlnI-Dral 1056bp fragment was gel purified. This fragment was ligated with a synthetic pair of 68 bp DraI-SalI kinased oligos (DRS1+2; SEQ ID NOS: 9797 & 9798) into a pT7Blue2 BlnI-SalI vector (FIG. 61). E. coli HB101 competent cells were transformed with the oligo replacement ligation product and plated on Luria agar plates, containing 100 μg/ml ampicillin. The desired clones were identified using miniscreen DNA analysis. After sequence confirmation the clone was named pT7Blue2 BInI-Sal Spike 1195 #7. The 1126 bp BlnI-SalI fragment encoding the 3′ end of the Spike 1195 was gel purified (FIG. 61).

In order to generate the XbaI-SalI Spike 1195 fragment, the 3109bp XbaI-PciI fragment was isolated from the p893-1.SARS Spike 1255 #9 (described above) and a 457bp PciI-SalI fragment from pT7Blue2.SARS Spike 1195 #7 (described above). The two fragments were cloned into the p893-1 XbaI-SalI vector, creating the p893-1.SARS Spike 1195 #34 plasmid (FIG. 62).

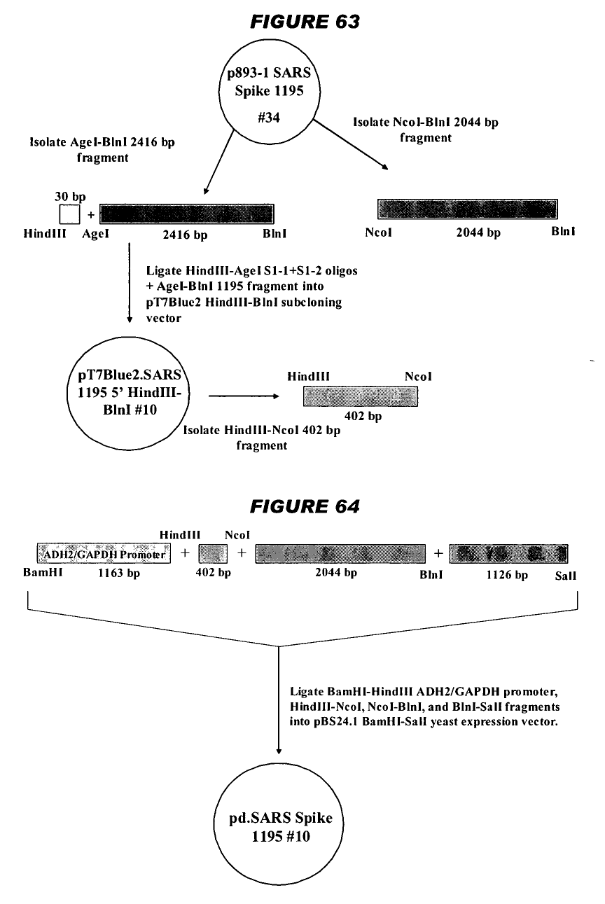

To clone SARS Spike 1195 into the pBS24.1 Saccharomyces cerevisisae expression vector, it was necessary to modify the 5′ end of the SARS Spike 1195 from XbaI to HindIII, as done for the Spike S1 expression clone described above. To begin, the 2416 bp AgeI-BlnI fragment was isolated from p893-1.SARS Spike 1195 #34. This fragment was ligated with the synthetic HindIII-AgeI 30 bp oligos (described above to generate the S1 protein for expression in S. cerevisiae) into the pT7Blue2 HindIII-BlnI vector. E. coli HB101 competent cells were transformed with the oligo replacement ligation product and plated on Luria agar plates, containing 100 μg/ml ampicillin. The desired clones were identified using miniscreen DNA analysis. After sequence verification of the positive clone and plasmid amplification of pT7Blue2.SARS 1195 5′ HindIII-BlnI #10 (FIG. 63), we isolated a 402 bp HindIII-NcoI fragment and the 2044 bp NcoI-BlnI fragment (FIG. 63). It was necessary for the HindIII-BlnI isolation to be done in two steps to avoid cloning issues related to the internal HindIII site located at nucleotide number 1319 of the spike 1195 protein.

To assemble the BamHI-SalI expression cassette of Spike 1195 into the pBS24.1 vector E.coli HB101 competent cells were transformed with the the BamHI-HindIII (ADH2/GAPDH promoter), HindIII-NcoI 402bp fragment, NcoI-BlnI 2044 bp and the BInI-Sall 1126 bp fragments into the pBS24.1 BamHI-SalI vector. The samples were plated on Luria agar plates, containing 100 μg/ml ampicillin. The desired clone was identified using miniscreen DNA analysis, thus creating the genetically engineered pd.SARS Spike 1195 #10 (FIG. 64).

S.cerevisiae strain AD3 was transformed with pd.SARS Spike 1195 #10 and single transformants were checked for expression after depletion of glucose in the medium. The recombinant protein was detected by Coomassie blue staining. In particular, yeast cells were transformed with the SARS 1195 expression plasmid using the Invitrogen S.c. EasyComp™ Transformation Kit.

### Example 23

### Expression in Mammalian Cell Lines

cDNA fragments containing the S protein ORF of 1255 amino acids were amplified by RT-PCR from SARS viral RNA (Frankfurt isolate) grown in Vero cells. The amplified PCR fragments were cloned into pBlueScript vector, sequenced, and consensus spike sequence was assembled to create a full-length SARS spike clone, pBSnSh. In vitro transcription of pBSnSh followed by translation in a rabbit reticulocyte lysate resulted in the production of single polypeptide with an estimated molecular mass of ˜140 kDa.

The insert of this plasmid was recloned via XhoI and Not I into a mammlian expression vector pCMVIII (Srivastava et al. (2003) J. Virol. 77:11244-11259) to create a construct, nSh (FIG. 74A). A PCR fragment containing a spike protein of 1195 amino acid, which was deleted for transmembrane (TM) domain and cystein-rich cytoplasmic tail (Cy) was amplified and cloned pCMVIII vector to generate the contstruct nShATC (FIG. 74B). Both constructs were tagged with six histidine residues at the C-terminus in order to aid in their characterization. The Xho I/Not I fragment without a histidine tag also was subcloned into the alphavirus replicon vector backbone pVCRchim2.1 for use in the production of an alphavirus replicon particle chimera that expresses S protein. Production and characterization of the replication defective alphavirus vector particles was performed essentially as described previously (Perri et al. (2003) J. Virol. 77:10394-10403; Polo et al. (1999) PNAS USA. 96:4598-4603). The resultant alphavirus vector particles were named as VEE/SIN.

COS7 cells and BHK-21 cells were maintained in Dulbecco's modified Eagle's medium supplemented with 10% fetal bovine serum at 37° C. and 5% CO₂ in air. COS7 cells were transfected with expression plasmids (nSh, nShΔTC) using a transfection kit (TransIt-COS, Mirus) following the manufacturer's protocol. The cells were washed once with ice-cold PBS and lysed with 1× Lysis buffer (20 mM MOPS, 10 mM NaCl, 1.5 mM MgCl₂, and 1% Triton X-100) containing complete mini protease inhibitor (Roche). After a 30-min incubation on ice, the debris was cleared by centrifugation. The cleared lysate was either purified or used directly in western blotting.

To purify secreted spike proteins, medium from transfected cells was collected and subjected to centrifugation at 12,000 rpm for 10 min to remove cellular debris. The cleared medium was applied to a ConA-agarose column (Vector Lab). The column was washed extensively with 20 mM sodium phosphate buffer, and then the bound proteins were eluted with 1M methyl α-D-mannopyranoside (MMP), 1M NaCl in 20 mM sodium phosphate buffer. Column fractions containing SARS-CoV spike proteins were applied to MagneHis Protein purification system (Promega) following the protocol suggested by the manufacturer.

For western blot analysis, proteins were separated by 4-20% SDS-PAGE and then transferred electrophoretically to nitrocellulose membrane (Invitrogen). Membrane was blocked in blocking buffer (5% skim milk and 0.1% Tween 20 in PBS) and incubated with indicated antibody at room temperature for 1 hr, washed and probed with horseradish peroxidase (HRP)-conjugated secondary antibody (Biosource) followed by chemiluminescence (ECL system, Amersham) and exposed by X-ray films. The antibodies used were a mouse monoclonal anti-histidine antibody (anti-His•tag Mab, Novagen), a rabbit polyclonlal antipeptide antibody against SARS-CoV spike proten (SmPab, Abgent), or rabbit anti-SARS sera (2BE) obtained by immunization of rabbits with purified SARS-CoV virion. The latter has a cell culture neutralizing titer of 1/2,500. Unless stated otherwise, antibody was used at 1/1,000 for anti-histidine antibody and SmPab and 1/10,000 for anti-SARS rabbit sera.

Some spike proteins were treated with Peptide-N glycosidase F (PNGase F). Cell lysates were diluted in 0.5% SDS and 1% β-mercaptoethanol and denatured at 100° C. for 10 min. After 2-fold dilution with 1% NP-40 in 50 mM sodium phosphate (pH 7.5), the samples were treated with PNGase F (NEB) at 37° C. for 1 hr. Enzyme-treated samples were analyzed by 4-12% SDS-PAGE in reducing condition. For a partial digestion with the PNGase, the cell lysates were diluted with 50 mM sodium phosphate (pH 6.0) containing 0.75% Triton-X and treated with PNGase F (Calbiochem) at 37° C. for 3 hr. Enzyme-treated samples were analyzed by 4-20% SDS-PAGE in nonreducing condition.

Western blots of cells 48-hours after transfection are shown in FIG. 75. The S protein was detected in cell lysates as a doublet with estimated molecular weight of ˜170-180 kDa, when the lysate was boiled and analysed under reducing SDS-PAGE conditions (FIG. 75A, lane 3). This doublet appears to result from differential glycosylation of one polypeptide product since pre-treatment of the cell lysate with PNGase F reduced the doublet to a single species of ˜140 kDa (FIG. 75A, lane 4). This is the expected size predicted from the aa sequence for a full-length, intact polypeptide product. This experiment indicates that the full length SARS-CoV S is expressed in mammalian cells as a single, uncleaved polypeptide, but in two differentially glycosylated forms, gp170 and gp180 respectively. Unlike the two S glycoforms encoded by the full-length sequence, none of which were secreted, the SΔ protein product was detected both in cell lysates (FIG. 75A, lane 5) as well as in the cell culture medium (FIG. 75B, lane3) as a single species of 160 kDa.

In order to further characterize the intracellular processing of the S protein, and as described above, BHK21 cells were infected with defective alphavirus particles expressing the full-length S. At 6 hr post infection with a MOI of 5, infected cells were pulse labeled for 1 hr with L-[³⁵S] methionine/cysteine and chased for 2 or 4 hours. The [³⁵S]-labeled S protein was immuno-precipitated using the rabbit antiserum raised against inactivated, purified virus and then digested with Endo H. The Endo H treatment involved dilution with a sample buffer (50 mM sodium phosphate, 0.1% SDS, 50 mM DTT, pH 6.0) and boiling for 5 min. After denaturation, the samples were further diluted with 0.75% Triton-X 100 and treated with endoglycosidase H (Endo H) following manufacturer's protocol (Calbiochem) for 3 hr at 37° C. Enzyme-treated samples were added with gel loading buffer containing 0.1% SDS and DTT and analyzed by 8% SDS-PAGE.

Both digested and undigested proteins were boiled in SDS and analysed by reducing SDS-PAGE (FIG. 55). After a 1-hr pulse, the S protein was apparent as a single gp170 component that was Endo H sensitive (lanes 1 and 2). After a 2-hr chase, a new species (gp180) was present along with gp170 in approximately equal proportions (lane3). After a 4-hr chase, the gp180 species was the dominant S protein component (lane 5) that was now Endo H resistant (lanes 5 and 6). This data is consistent with gp170 being an ER-resident glycoprotein containing high mannose chains and with gp180 corresponding with a Golgi-processed glycoprotein containing Endo H-resistant complex oligosaccharides.

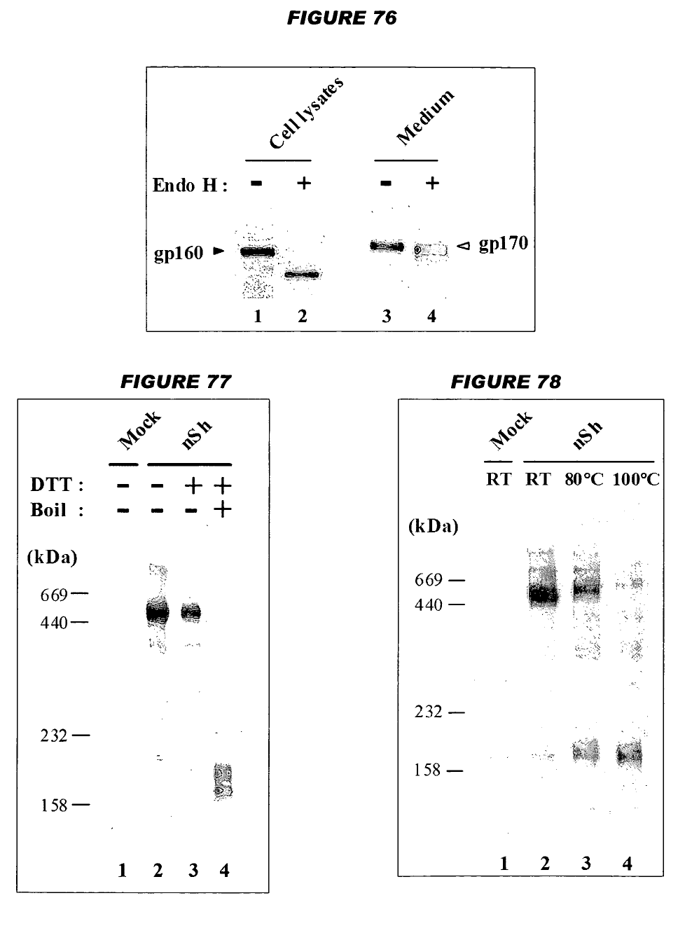

The Endo H sensitivity of the C-terminus deleted SΔ protein purified from cell culture media ws also tested. As shown in FIG. 76, the SΔ observed within cell lysates was found to be Endo H sensitive (lanes 1 and 2), while the secreted SΔ in cell culture media was Endo H resistant (lanes 3 and 4). This result is consistent with this glycoprotein being synthesized in an immature form in the ER prior to transfer to the Golgi where the complex carbohydrate is added and the protein then secreted.

As already described, the S protein expressed in COS7 cells was detected as a gp170/gp180 doublet in western blot analyses of cell lysates that were fully denatured by boiling in the presence of DTT. However, the majority of S protein was detected as a high molecular glycoprotein in the 440-669 kDa range when the same cell lysate was not heat-denatured prior to western blot analysis using SDS-PAGE (FIG. 77, lane 1). The ˜500 kDa species was resistant to 10 mM DTT treatment (lane 3) and not dissociated into the monomeric form unless the lysate was first heat-denatured at 100° C. (lane 4). In contrast, oligomeric form of a test protein (Thyroglobulin) of which quaternary structure is held by disulfide-linkage was converted into subunit form by the 10 mM DTT treatment. These data suggest that the ˜500 kDa oligomeric form of S protein is not disulfide-linked and is heat labile. To confirm the heat-sensitivity of the ˜500 kDa species of S protein, the heat-denaturation experiment was repeated but without DTT. As shown in FIG. 78, heat denaturation of 500 kDa protein at 100° C. alone was sufficient to convert it into gp170/180 monomeric forms (lane 4). Using a 80° C. heat-denaturation step, both the ˜500 kDa and monomeric forms were detectable in similar proportion (lane 3).

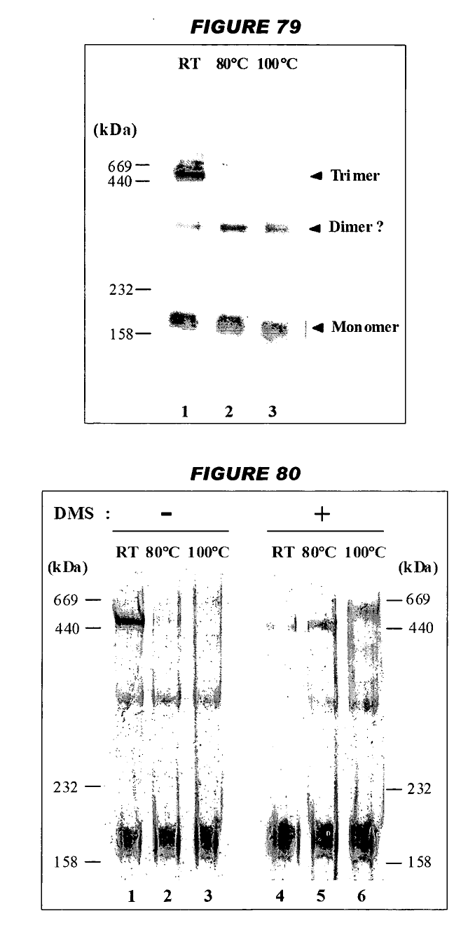

In order to investigate further whether this ˜500 kDa species represents an S protein oligomer in native conformation, comparative analyses with virion-derived S glycoprotein derived from Vero cell cultures was performed. The purified virions were solubilised in 1% SDS prior to Western blot analyses after SDS PAGE. The presence of the ˜500 kDa spike protein oligomer was confirmed in virion particles (FIG. 79, lane 1). In addition, heat denaturation of solubilised virions produced the same oligomer-to-monomer conversion as seen with the full-length recombinant S (lanes 2,3). The oligomeric nature of virion S was further analysed in a cross-linking experiment. Aliquots of inactivated virion from sucrose gradient fractions were treated with 10% SDS at 1% final concentration and diluted 2-fold with 0.2M Triethanolamine-HCl (pH 8, Sigma); Dimethyl suberimidate (DMS; Pierce Chemical Co.) was then added from a freshly prepared solution (10 mg/ml in 0.2M Triethanolamine-HCl) at 3.3 mg/ml final concentration. After 2 hr at room temperature, samples were concentrated with Centricon-30 and analyzed by silver staining after electrophoresis on a 4% polyacrylamide gel. Both untreated and DMS cross-linked virion proteins were heat-denatured, and the heat effect on the maintenance of oligomer structure was analysed by SDS-PAGE and silver staining (FIG. 80). In the absence of cross-linking, heat denaturation resulted in the replacement of the ˜500 kD spike protein species with the monomer species. In contrast, in the cross-linked proteins, the levels of the ˜500 kD and monomer species did not change significantly after heating. These data support the fact that the ˜500 kD protein is an oligomer of S monomer proteins that are bound non-covalently. After cross-linking and boiling, the ˜500 kDa species migrated as a somewhat slower diffuse form than the untreated form. This mobility shift is probably due to a structural change resulting from boiling. In addition, a minor protein species of ˜300 kD, which may represent a non-dissociated S dimer, could be seen.

To estimate more precisely the size of the recombinant ˜500 kDa S species expressed in COS7 cells, a COS7 cell lysate containing the S protein oligomer was fractionated using size-exclusion column chromatography. The major portion of the ˜500 kDa oligomer co-eluted with a 572 kDa marker protein. Taken together, these experiments suggest that the ˜500 kDa S species seen in COS7 cell lysates is probably a homotrimer of the S protein monomer.

The oligomeric status of the SΔ spike protein was also examined after expression in COS7 cells. As shown in FIG. 81, the recombinant SΔ proteins present in cell lysates were also detected in high molecular weight forms of ˜500 kDa range when the lysate was not heated prior to SDS-PAGE and Western blot analysis (lane 1). However, the efficiency of oligomerization by intracellular SΔ protein appears to be much less (<10%) compared to that of full-length S protein under the same western analysis conditions. A heat-sensitivity test on this ˜500 kDa protein showed that the SΔ oligomer was more heat labile than that of the full-length S oligomer, as demonstrated by the >90% conversion of all of the ˜500 kDa species into monomeric Sd forms at 80° C. (lane 2). Also (FIG. 82), the majority of the secreted SΔ protein was found in monomeric form with the ˜500 kDa species barely detectable (and only detectable when the protein was loaded in excess for Western analysis) (lane 1). At a temperature above 80° C., all secreted SΔ proteins were detected as monomers (lanes 2, 3).

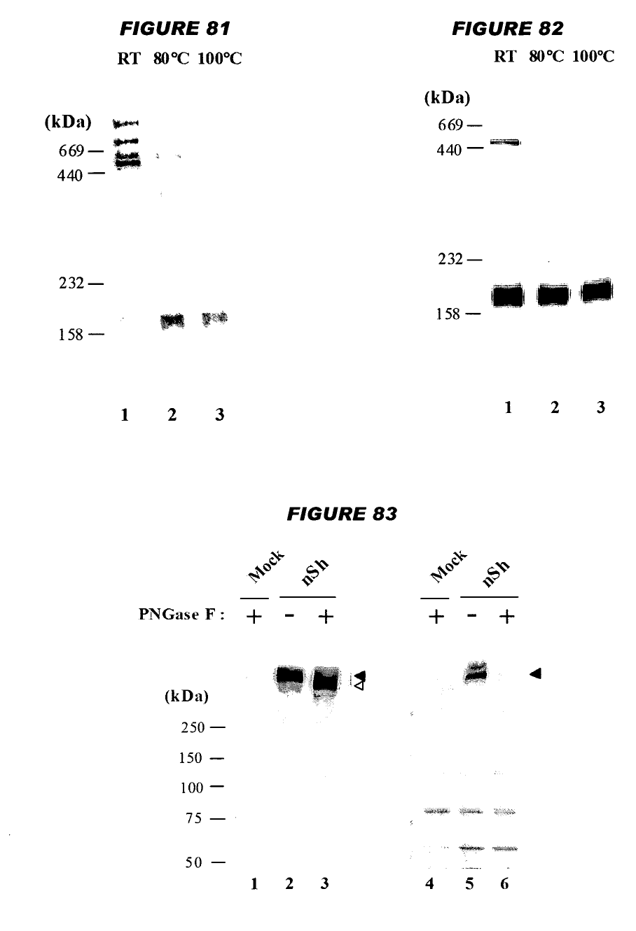

The ˜500kDa protein is glycosylated, and the effect of deglycosylation on its antibody binding was investigated. The recombinant COS7 lysate was treated with PNGase F under non-denaturing condition (as described above) and analysed by western blot. As shown in FIG. 83, deglycosylation did not affect the binding of anti-histidine Mab antibody to the treated S oligomer (lanes 2,3). However, it compromised the reactivity with the rabbit antisera raised against purified virus (lane 6). This antiserum binds to virion-derived S in western blot analyses only when DTT is omitted from the sample for SDS-PAGE indicating that it recognizes primarily a discontinuous, conformational epitope(s). This antisera has also been shown to have a high-titer of viral neutralizing antibodies. Its lack of binding to deglycosylated, recombinant S suggests that the carbohydrate actively contributes to the higher order, native structure of the S polypeptide oligomer.

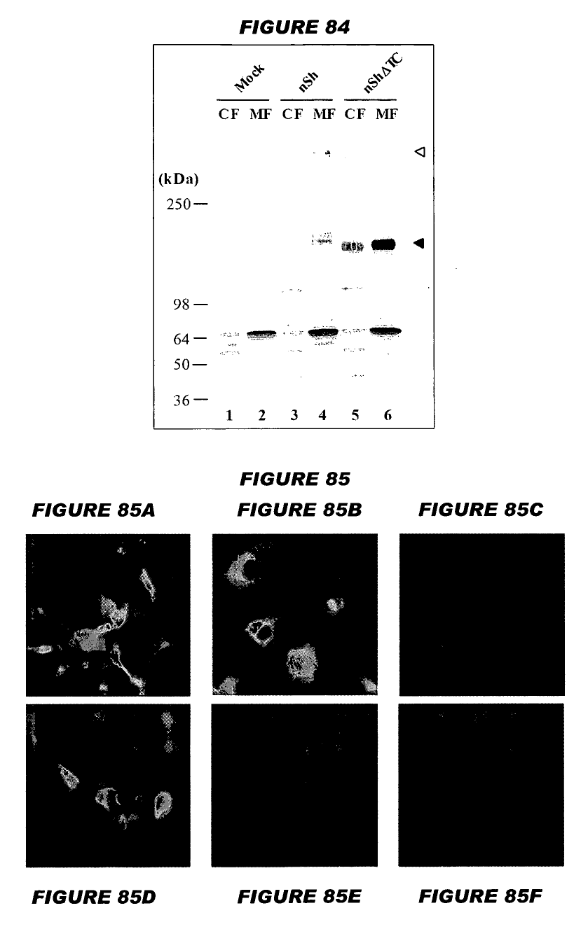

The difference between the recombinant S and SΔ protein is the presence or absence of the TM-and Cys-rich domains at the C-terminus. This difference predicts that full-length S would be found associated with the membrane fraction while Sd would be in the soluble fraction upon lysis of transfected cells. Therefore, nSh- or nShΔTC-transfected cells were lysed under hypotonic conditions and the soluble cytosol fraction was separated from the insoluble membrane fraction by centrifugation (FIG. 48). As shown in FIG. 84, the S protein was found in the membrane fraction (DF) both as a ˜500 kDa and 180/170 kDa species (lane 4) but was not detectable in the soluble cytosol fraction (AF) (lane 3). However, the truncated SΔ protein was found as a monomeric species (gp170) in both fractions (lanes 5,6). This indicates that the C-terminal TM and Cys-rich domains are required for the anchorage of the S protein to cell membrane.

The cellular location of the S and SΔ proteins in COS7 cells was analyzed by indirect immunofluorescence microscopy. At 48 hr post-transfection, cells were directly fixed with 2% paraformaldehyde without detergent for cell surface staining or treated with detergent followed by Cytofix/Cytoperm solution for intracellular staining. Fixed cells were then stained with rabbit anti-SARS sera (2BE) and FITC-conjugated antibody. The nSh-transfected cells showed foci of S protein indicative of Golgi-localisation (FIG. 85A), while the nShΔTC-transfected cells displayed a uniform distribution of SΔ protein throughout the cytoplasm indicative of ER localisation (FIG. 85B). While the complete S protein was also observed on the surface of transfected cells in unfixed cells (FIG. 85D), the SΔ was undetectable on the cell surface (FIG. 85E). These results indicate the role played by the TM-and Cys-rich domains in anchoring the S protein to the plasma membrane. Although the TM-region alone is likely responsible for membrane anchorage, the potential role played by the Cys-rich region remains to be determined.

The SARS recombinant full-length S protein is thus an N-linked glycoprotein with an estimated molecular weight of 170-180,000 kDa. Deglycosylation with PNGase F resulted in a polypeptide of the expected size for the uncleaved, encoded polypeptide (140 kDa). Both transient and stable expression of the full-length SARS-CoV S gene in a variety of mammalian cells, including COS7, 293, BHK21, and Huh7 cell lines, consistently produced a S protein doublet (gp170/180) as detected in western blot analyses. Pulse-chase analyses of transfected cells demonstrated that the SARS CoV S protein was initially synthesized as an Endo H sensitive gp170 species followed by the gradual appearance of an Endo H resistant gp180 form, presumably as a result of the addition of complex carbohydrate within the Golgi apparatus.

The recombinant S protein was not secreted into the cell culture medium unless the C-terminal 60 amino acids containing the TM-region and the Cys-rich tail were deleted.

The quaternary structure of the full-length recombinant S protein was investigated using cross-linking treatment, heat-denaturation, and size fractionation analyses. The results data are consistent with the recombinant S protein existing as a homotrimer of ˜500 kDa. Similar analyses of virion-derived S yielded the same results. Such a trimeric structure has been reported for other enveloped RNA viruses: the hemagglutinin HA of influenza virus, the E1-E2 heterodimer of alphaviruses and the G protein of vesicular stomatitis virus. Incubations under reducing conditions indicate that the SARS-CoV S trimeric structure is non-covalently associated, and is very stable. S oligomers present in the cell lysate were shown to be resistant to reduction by 10 mM DTT, detergent treatment with 1% SDS, and heat denaturation at up to 60° C. . Incubation at a temperature higher than >80° C. resulted in the dissociation of the trimeric complex as evidenced by the decrease in trimer with the concomitant increase in the monomer bands. The temperature-induced appearance of the high-mannosylated gp170 (ER monomer form) as well as the complex-glycosylated gp180 (Golgi monomer form) suggests that trimerization can occur before the transport of the monomer spike protein to the medial Golgi apparatus. This is consistent with other reports for TGEV, influenza virus HA, and vesicular stomatitis virus G proteins. With these proteins, trimerization was reported to take place before addition of complex oligosaccharides in the Golgi apparatus.

The C-terminally truncated form of S was found in the cell lysate in both oligomeric and monomeric forms at a frequency of 10% and 90%, respectively. The truncated protein secreted into medium was found fully glysosylated and it was essentially all in monomeric form. We conclude that the C-terminal 60 amino acids of the S glycoprotein contains a membrane anchor region that affects the efficiency of trimerization. In S protein trimerization, it is possible that the C-terminal region is required to initiate the event and the triple-stranded coiled coil structures in the S2 stalk domain provide further stabilizing force as seen in HA oligomer of influenza virus.

### Example 24

### CHO Cells for Spike Protein Expression

CHO cell lines that stably express either the full-length or truncated SARS-CoV spike proteins were prepared. Several stably transfected CHO cell lines were obtained, and FIG. 73 shows western blot data from a panel of representative clones.

### Example 25

### Expression in E.coli

All SARS-CoV ORFs (FIG. 17, Table 10) were cloned in the pET vector and expressed as C-terminal His-Tag fusion proteins in E. coli. The proteins smaller than 16 KD were also expressed as N-terminal GST (Glutathione S-transferase) fusion proteins using pGEX vector.

Nsp1 and Nsp2, the two SARS-CoV proteins with proteolytic activity, were not expressed as full length proteins due to toxicity in E. coli. The respective genes were instead cloned in different portions in order to separate the catalytic residues (Cys833/His994 for Nsp1; His41/Cys145 for Nsp2) in the resulting recombinant proteins: Nsp1A from nucleotides 2719-5214 of AY310120; Nsp1B from nucleotides 5218-7371; Nsp1C from nucleotide 7372-9984; Nsp2A from nucleotide 9985-10416; Nsp2B from nucleotide 10476-10902.

Nsp9 (SEQ ID NO: 9775) was divided into two portions: Nsp9A from nucleotide 13371-14756; Nsp9B from nucleotide 14757-16166.

Matrix (M), ORF3 and ORF7 contain respectively three, two and one transmembrane domains. These proteins were expressed as deletion proteins excluding the first 100 amino acids (M and ORF3) or the first 18 amino acids (ORF7) that include the hydrophobic regions.

The cloned sequences are shown in Table 26.

A two-step strategy was used to amplify the cloned sequences. In the first step, amplification of DNA fragments containing more than one gene or single gene used sequenced cDNA as template. Eleven cDNA sequences were amplified: (1) a fragment, named amplC1, including genes coding for protein E, protein M, orf 7-8-9-10; (2) a fragment, named amplC2, including genes coding for orf 3-4; (3) a fragment, named amplC5, including genes coding for proteins Nsp12 and Nsp13; (4) Nsp11gene; (5) P28 and P65 genes; (6) Nsp1B and Nsp1C genes portion; (7) a fragment, named amplC9, including genes coding for proteins Nsp2 and Nsp3; (8) a fragment, named amplNsp4-7, including genes coding for proteins Nsp4, Nsp5, Nsp6, Nsp7 and for amplification of Nsp9A gene portion; (9) Nsp 9B gene portion and Nsp10 gene; (10) a fragment, named amplCO, including genes coding for proteins Orf11, Nucleocapsid (N) and Orf12; (11) Nsp1A gene portion. The primers used in this first step are given in Table 27:

In the second step, amplification of single genes was performed using DNA fragments from the first amplification step as templates. The primers are shown in Table 28.

Of the proteins where expression was seen, it was either in inclusion bodies (insoluble) or in a soluble form. Purification proceeded on appropriate material. Table 29 shows the molecular weight of the expressed fragments of SARS-CoV ORFs, whether they were cloned (+ or −), whether the cloned fragment was seen to be expressed (+ or −) and the form of protein which was chosen for purification.

Where a protein was a soluble His-tagged product, a single colony was streaked and grown overnight at 37° C. on a LB/Amp (100 μg/ml) agar plate. An isolated colony from this plate was inoculated into 20 ml of LB/Amp (100 μg/ml) liquid medium and grown overnight at 37° C. with shaking. The overnight culture was diluted 1:30 into 1.0 L LB/Amp (100 μg/ml) liquid medium and allowed to grow at the optimal temperature (30 or 37° C.) until the OD550 nm reached 0.6-0.8. Expression of recombinant protein was induced by addition of IPTG (final concentration 1.0 mM) and the culture incubated for a further 3 hours. Bacteria were harvested by centrifugation at 8000×g for 15 min at 4° C. The bacterial pellet was resuspended in 10 ml of cold buffer A (300 mM NaCl, 50 mM phosphate buffer, 10 mM imidazole, pH 8.0). Cells were disrupted by sonication (or French Press) on ice four times for 30 sec at 40 W using a Branson sonifier 450 and centrifuged at 13 000× g for 30 min at 4° C. Supernatants were mixed with 150 μl Ni²⁺-resin (previously equilibrated with buffer A) and incubated at room temperature with gentle agitation for 30 min. The resin was Chelating Sepharose Fast Flow (Pharmacia), prepared according to the manufacturer's protocol. The batch-wise preparation was centrifuged at 700×g for 5 min at 4° C. and the supernatant discarded. The resin was washed twice (batch-wise) with 10 ml buffer A for 10 min, resuspended in 1.0 ml buffer A and loaded onto a disposable column. The resin continued to be washed with buffer A at 4° C. until the OD280nm of the flow-through reached 0.02-0.01. The resin was further washed with cold buffer B(300 mM NaCl, 50 mM phosphate buffer, 20 mM imidazole, pH 8.0) until the the OD280 nm of the flow-through reached 0.02-0.01. The His-fusion protein was eluted by addition of 700 μl of cold elution buffer C (300 mM NaCl, 50 mM phosphate buffer, 250 mM imidazole, pH 8.0) and fractions collected until the OD₂₈₀ₘₘ indicated all the recombinant protein was obtained. 20 μl aliquots of each elution fraction were analyzed by SDS-PAGE. Protein concentrations were estimated using the Bradford assay.

Where a protein was seen as an insoluble product, the inclusion bodies were purified as follows: homogenize cells (5 g wet weight) in 25 ml 0.1M Tris HCl pH 7, 1 mM EDTA, at 4° C. using an ultraturrax (10000 rpm); add 1.5 mg lysozyme per gram cells; mix shortly with an ultraturrax and incubate at 4° C. for 30′; use sonication or high-pressure homogenization to disrupt the cells; to digest DNA, add MgCl₂ to a final concentration of 3 mM and DNase to a final concentration of 10 ug/ml and incubate 30′ at 25° C. add 0.5 vol of 60 mM EDTA, 6% Triton x-100, 1.5M NaCl pH 7.0 to the solution, and incubate for 30′ at 4° C.; centrifugation at 31000 g for 10′ at 4° C.; re-suspend pellet in 40 ml of 0.1M Tris HCl pH 7.0, 20 mM EDTA using ultraturrax; centrifugation at 31000 g for 10′ a 4° C.; store the IB pellet at −20° C.

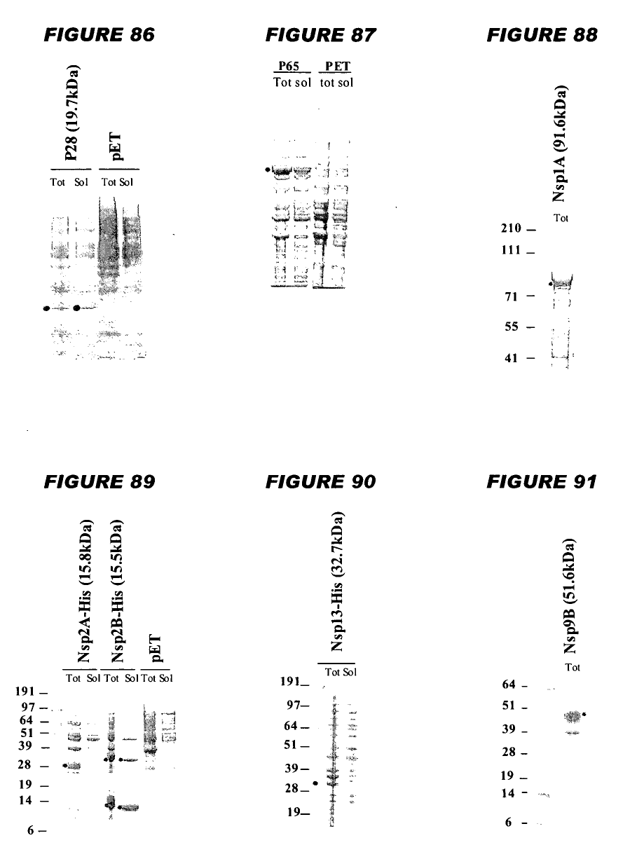

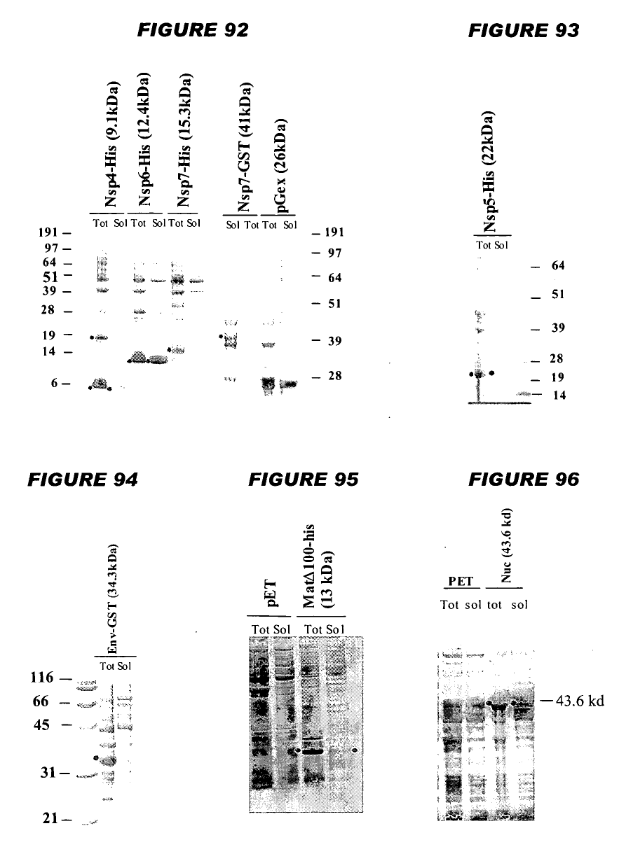

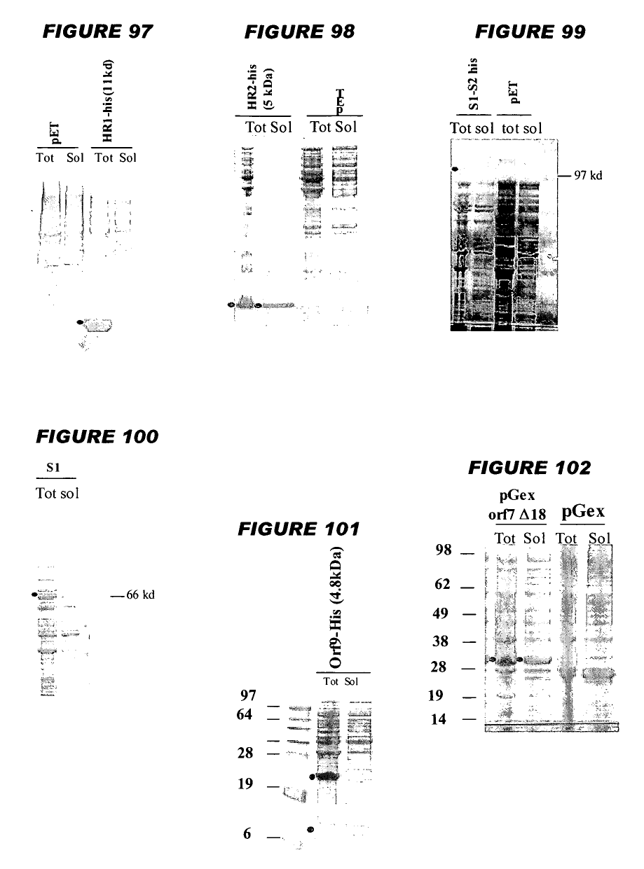

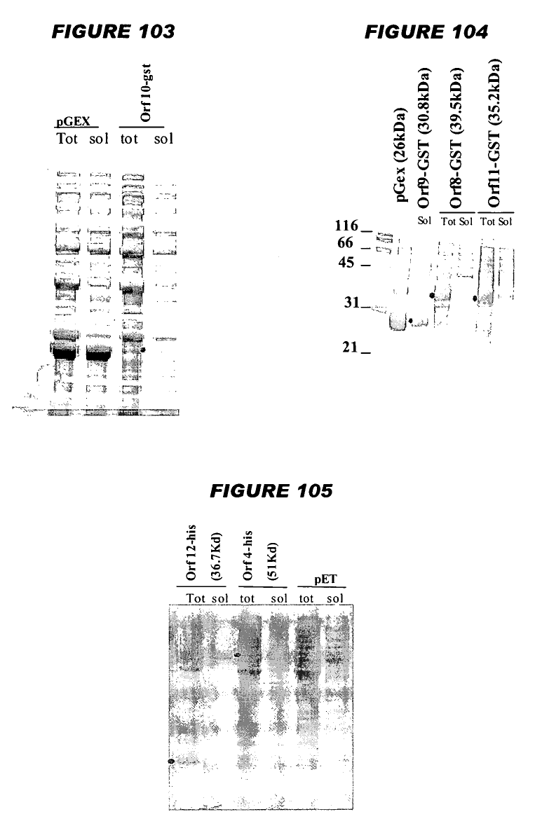

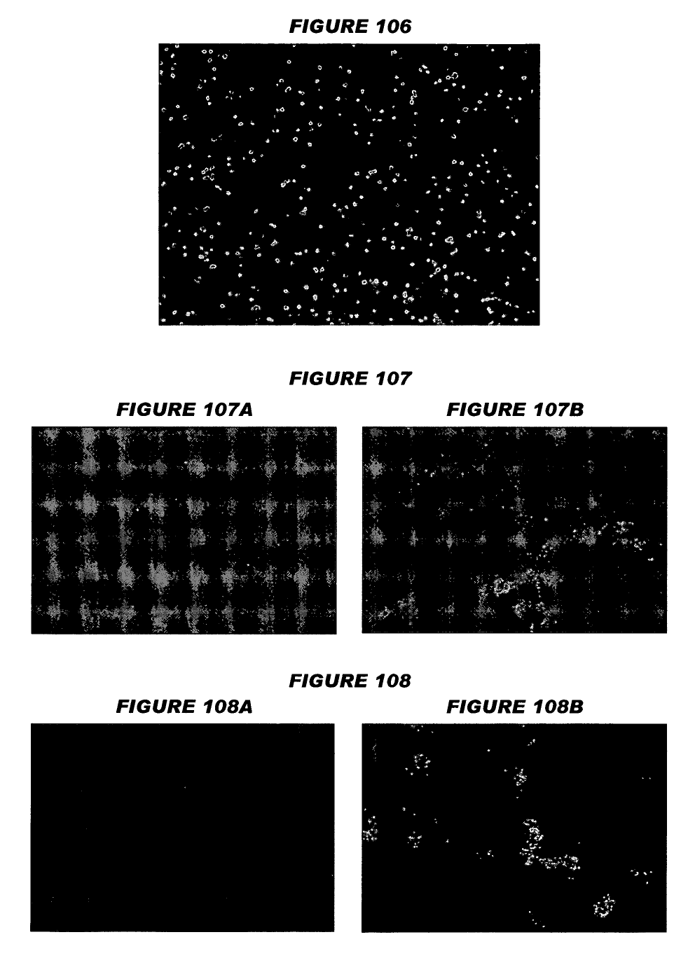

The results of expression are shown in FIGS. 86 to 105. Examples of purity and yield are given in Table 30.

### Example 26

### Retention of Critical Epitope on Truncated Spike Antigen

A human monoclonal antibody having neutralizing activity was tested in an ELISA assay for reactivity with the purified truncated Spike protein. Briefly, ELISA plates were coated with truncated form of the spike protein at a concentration of 1 μg/ml (100 μ/well) by incubating the plates overnight at 4° C. The plates were washed, non-specific binding sites were blocked and then different dilutions of the antibody were added and plates were incubated for 1 hour at room temperature. At the end of incubation, the plates were washed and bound antibody was detected by using anti-human IgG conjugated to horse radish peroxidase (HRP) and an appropriate substrate. The optical density of each well was recorded at 405 nm using an ELISA reader. The data are shown in FIG. 69 and clearly demonstrate that the neutralizing epitope recognized by the mAb is preserved and exposed on the recombinant truncated Spike protein.

### Example 27

### Different Spike Vaccines

Purified truncated spike protein was used to immunize mice and the level of binding antibodies induced against the truncated spike protein was determined by ELISA assay. Briefly a group of 10 mice were immunized with 3 μg of truncated spike protein adjuvanted in MF59 at 0, 4 and 8 weeks intervals. Sera samples were collected from these animals and assayed for antibodies induced by truncated spike protein in an ELISA assay. An additional group of 8 mice was immunized with 75 jig of DNA encoding the truncated form of the spike protein on PLG particles at 0, 4 and 13 weeks intervals, the sera were collected and analyzed as above for anti-spike antibodies as above

The profile of binding antibodies induced in each group was plotted as geometric mean titer (GMT). Compared to a plasmid DNA vaccine expressing truncated spike antigen and delivered using a PLG microparticle formulation, the purified truncated spike protein was significantly more potent for inducing strong antibody responses. Further comparison with the antibody responses induced by inactivated BPL-SARS-CoV (already shown protective) in the same mouse strain revealed that the magnitude of antibody responses induced by purified truncated spike protein and the inactivated virus vaccine are in the same range (FIG. 70).

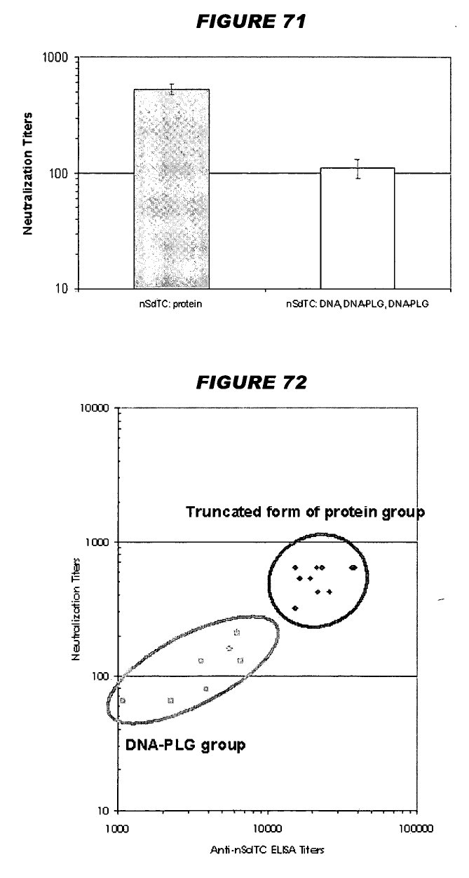

The neutralization potential of antibodies induced by the recombinant truncated spike protein, or plasmid DNA expressing the same spike antigen, were also evaluated. The GMT values obtained in both groups are shown in FIG. 71. From these data, it appears that the purified protein is significantly more effective at inducing neutralizing antibody responses against the SARS-CoV spike. Furthermore, the neutralization titers typically induced by the purified truncated spike protein are comparable to neutralization titers induced by an inactivated SARS-CoV vaccine.

FIG. 72 shows a comparison of antibody binding levels (ELISA, X-axis) with neutralization titers (Y-axis). In general there is a very good correlation between the binding and neutralizing antibodies. The bottom-left grouping shows ratios 2 weeks post-3rd immunization with the DNA vaccine; the top-right grouping shows ratios 2 weeks post-2nd immunization with the protein vaccine. Both forms of vaccine show a consistent correlation.

In further experiments, the ability of a DNA vaccine to invoke an immune response in mice was studied. Mice were immunized with pCMV-nSdTC plasmid, either free or with PLG microparticles. Serum from the mice was then used as the staining antibody against cultured 293 cells that had been transfected with spike, either full-length or truncated. The cells were centrifuged prior to testing and the pellet was lysed. The antibody was tested against the culture supernatant and against the cell lysate. As shown in FIG. 112, the mouse serum was able to detect spike protein in the lysate of cells that expressed full-length spike and in the supernatant of cells that expressed the truncated spike protein. Results were comparable to the staining seen when using rabbit serum that had been obtained after immunization with whole killed virus. Thus anti-spike antibodies can be induced by the use of DNA vaccination.

### Example 28

### Expression Cassettes in pCMV

The sequence of plasmid pCMVKm2 is given as SEQ ID NO: 9923. Genes encoding the spike protein either in full-length form (pCMVKm2 SARS Spike nS; SEQ ID NO: 9921) or in its ΔTC form (pCMVKm2 SARS Spike nSΔTC; SEQ ID NO: 9922) were inserted into this basic vector.

Mice were immunized with these vectors, and with similar vectors encoding the N, M or E proteins. Vectors encoding the same proteins but with optimized codon usage were also prepared. Codons were optimized for efficient human expression starting from the FRA sequence (GenBank: AY310120). The optimized sequences are: N (SEQ ID NO: 9924); M (SEQ ID NO: 9925); E (SEQ ID NO: 9926).

After administration, expression of proteins could be detected by immunofluorescence in all cases. For example, FIG. 106 shows immunofluorescence (using anti-SARS rabbit serum) results after administration of the vector encoding optimsed N antigen, revealing high level expression. Mice receiving the control vector alone showed no fluorescence.

FIG. 107 compares immunofluorescence (using Abgent anti-M antibody) of the native M sequence (107A) or the codon-optimsed M sequence (107B). Similarly, FIG. 108 compares immunofluorescence (using Abgent anti-E antibody) of the native E sequence (108A) or the codon-optimsed E sequence (108B).

Four groups of mice (8 mice per group) were immunized with: (1) SARS nS Spike, nSdTC truncated Spike, and N proteins; (2) pCMV-SARS-nSdTC: DNA+DNA-PLG at weeks 0,4 and 13 wks; (3) CMV-nS: DNA+DNA-PLG+VEE/SIN Rep at 0, 4 and 9 wks; (4) VEE/SIN Rep-SARS-nS three times at 0, 4 and 13 wks. Sera from all groups recognized SARS nS and nSdTC proteins, and also showed virus binding and neutralization activity.

### Example 29

### Spike Protein Cleavage

To investigate the effect of proteolytic cleavage on SARS-CoV Spike protein, it was expressed in various forms in E.coli, including: (1) full-length S1-S2; (2) S1 alone; (3) HR1 heptad; and (4) HR2 heptad. The expressed proteins were used to raise immune rabbit sera, which were then used for visualizing western blots of Vero cells, either infected or not infected with SARS-CoV.

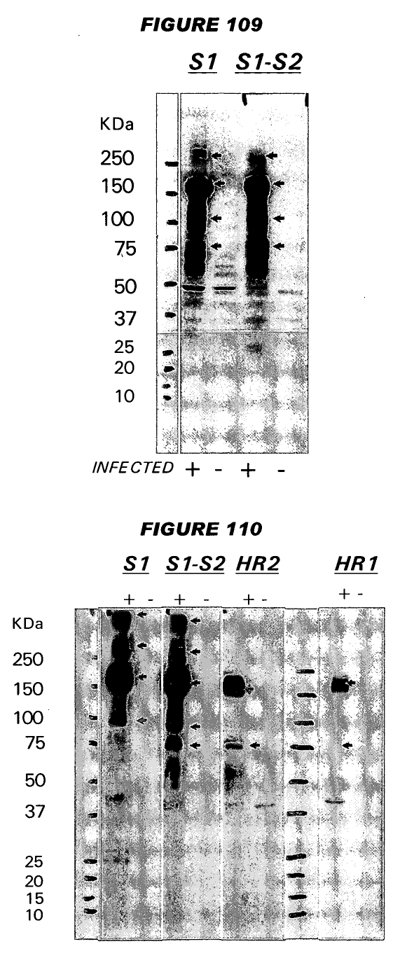

FIG. 109 shows a western blot using a 1:10000 dilution of antibody raised against either the S1 domain or the uncleaved S1-S2 domains. FIG. 110 shows a western blot using a 1:10000 dilution of antibody raised against each of the four proteins. The difference in antigen reactivity is readily apparent.

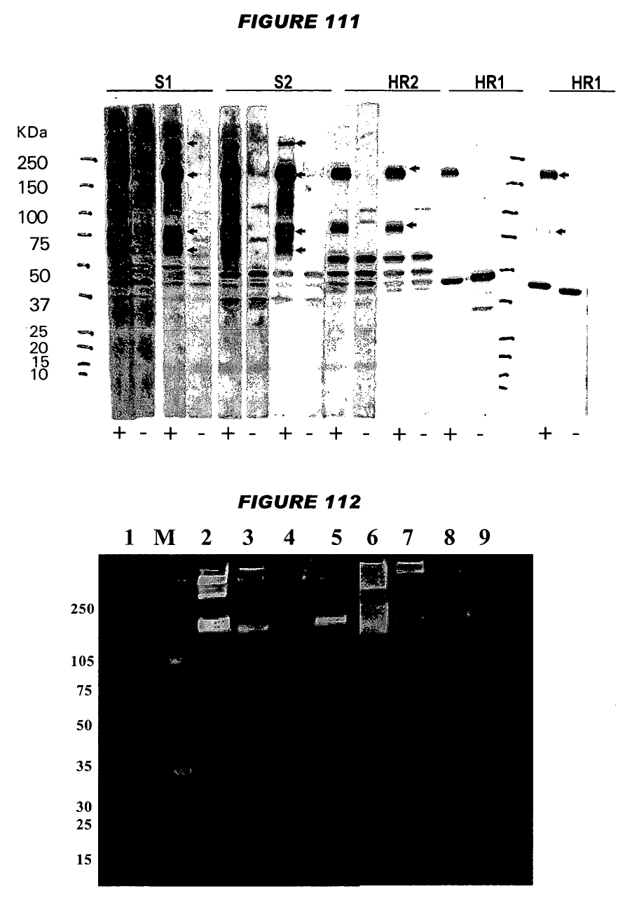

FIG. 111 shows similar data. Each serum was tested against four lanes, with those gour lanes being from left to right: (a) serum at 1:500 dilution, SARS-CoV-infected cells; (b) serum at 1:500 dilution, non-infected cells; (c) serum at 1:2500 dilution, SARS-CoV-infected cells; (d) serum at 1:2500 dilution, non-infected cells. Again, the difference in antigen reactivity is readily apparent.

FIGS. 109-111 show that the Spike protein exists in various forms in infected Vero cells, with sizes of approx. 75 kDa, 90 kDa, 180 kDa and >250 kDa. The Spike protein is cleaved (at least partially) either intracellulary or after release of the particles.

If enzymatic cleavage of the mouse hepatitis coronavirus spike protein is inhibited then cell-cell fusion (syncytia formation) is also inhibited, but virus-cell fusion is not (de Haan et al. (2004) J Virol). Syncytia are observed in vivo in the lungs of SARS-infected patients, but are not seen in Vero cell cultures of the SARS-CoV. Inhibition of Spike protein cleavage may thus be used to prevent syncytia formation and related pathology, even though viral infectivity may not be blocked.

### Example 30

### Purification of SARS Protease

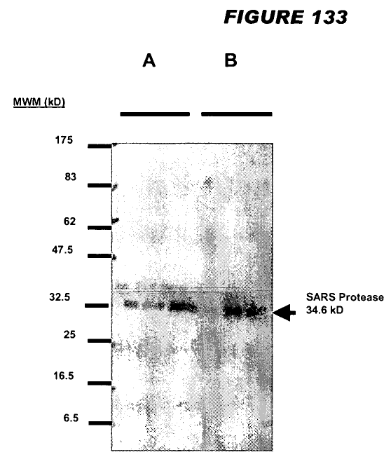

Cells were grown at 37° C. to mid-log phase and induced with 0.2% L-arabinose. Cells were harvested by centrifugation, and the cells resuspended in lysis buffer (LB) containing 20 mM Tris pH 7.5, 500 mM NaCl, 5% glycerol V/V, 0.05% Triton X-100, 5 mM βME, 5 mM imidazole, and complete protease inhibitors (−)EDTA. Benzonase was added to a final concentration of 50 U/ml of lysate. Cells were then lysed using two passes through a pre-chilled microfluidizer. The lysate was clarified by high speed centrifugation at 44,000× g. Clarified lysate was applied to a prepared Pharmacia chelating FF column charged with nickel sulfate. After application of the lysate the column was washed with 5 column volumes of LB, followed by 5 column volumes of LB supplemented with 45 mM imidazole. The column was then eluted using LB supplemented with 250 mM imidazole. Purity of the isolated SARS protease was 50%. Fractions containing protease were pooled, adjusted to 5 mM EDTA, and then applied to a Superdex 200 gel filtration column equilibrated in 20 mM Tris pH 7.5, 150 mM NaCl, 5% V/V glycerol, 0.05% Triton X-100, and 5 mM DTT. Purity of the isolated SARS protease was 70%. Again, fractions containing the protease were pooled, and then stored at −80° C. until used. Activity assay, mass spectrometry and western blot analysis were used to positively identify the protein (FIG. 133). All steps were carried out with pre-chilled buffers, and kept at 4° C. for as much of the preparation as possible.

Western of SARS Protease Purification Fractions

Protocol: Briefly, protein concentration was based on Absorbance at 280 nm, and coomassie stained gel estimates of purity. Protein was run on a 4-20% gradient gel, and transferred to nitrocellulose. The blot was then blocked with 3% BSA, probed with Mouse IgG anti-pentaHis, and then probed with a secondary antibody to Mouse IgG conjugated with HRP. The blot was visualized using an ECL kit (Pharmacia Biotech). The results are shown in FIG. 133 where A is the sizing column pool loaded at 50, 100 and 200 ng of target protein and B is the immobilized metal affinity column pool loaded at 50, 100 and 200 ng of target protein.

### Example 31

### Continuous Fluorescence Resonance Energy Transfer (FRET) Enzyme Assay

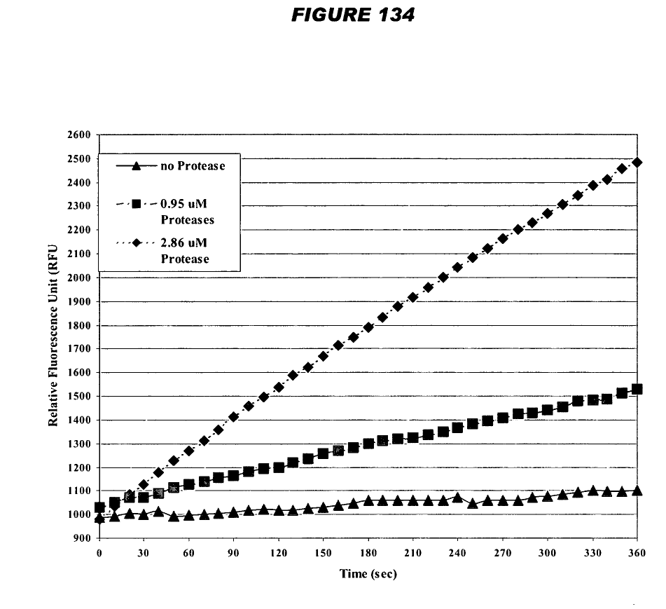

The peptide containing EDANS, the fluorescence donor, and DABCYL, the fluorescence quencher (DABCYL-VNSTLQ ∇SGLRK-EDANS) was synthesized by Syn. Pep. (Dublin, Calif.). The peptide contains the cleavage site Gln-Ser in the middle. Meyers, G. et al. Handbook of Proteolytic Enzymes and Barrett, A et al., Academic Press, London, 1998, 726-728. The proteolytic activity of SARS protease was followed kinetically by measuring the level of formation of cleaved product that contains the fluorescence donor, SGLRK-EDANS using the Hitachi fluorometer (F-4500 FL Spec.) set at 340 nm excitation and 490 nm emission wave length. 5 μL of 5 mM peptide stock in DMSO solution was added to the reaction mixture, containing 295 μl of standard buffer (75 mM Tris-Hcl, 25 mM NaOAc, 25 mM Bis-Tris, 25 mM glycine, 5 mM EDTA, and 1 mM EDTA, pH 7.4) and 100 ul of buffer or 100 ul of 3.6 uM protease stock solution. The kinetic curve was followed for 6 minutes (the reaction was linear with R2 value of 0.998 (FIG. 134)). The formation of fluorescence (proteolytic reaction) is likely enzyme dependent, as concentration of enzyme was tripled three times as much fluorescence was formed in the 6 minutes time frame.

It will be understood that the invention has been described by way of example only and modifications may be made whilst remaining within the scope and spirit of the invention.

TABLE 1
US Patents and Published International Patent Applications

| Publication | | Publication |
|---|---|---|
| Number | Title | Date |
| US-3927216 | 1,2,4-Triazol E-3-Carboxamides For Inhibiting Virus Infections | Dec. 16, 1975 |
| US-4010269 | Antiviral Quinazoline Compositions And Methods Of Use | Mar. 1, 1977 |
| US-4065570 | Antiviral 5-(Substituted Benzal) Hydantoins | Dec. 27, 1977 |
| US-4089965 | Thiazolylphenylguanidines As Antirhinovirus Agents | May 16, 1978 |
| US-4122191 | Antirhinovirus Agents | Oct. 24, 1978 |
| US-4192895 | Antirhinovirus Agents | Mar. 11, 1980 |
| US-4254144 | Substituted Benzonitriles Having Antiviral Activity | Mar. 3, 1981 |
| US-4264617 | Antiviral 5-(Substituted Benzal) Hydantoins | Apr. 28, 1981 |
| US-4287188 | Purine Derivatives | Sep. 1, 1981 |
| US-4327088 | Phosphonooxy- Or Glycosyloxy-Substituted Acrylophenones, Compositions And Uses Thereof | Apr. 27, 1982 |
| US-4332820 | Substituted Benzonitriles Having Antiviral Activity | Jun. 1, 1982 |
| US-4349568 | Sulfur-Substituted Diphenyl Ethers Having Antiviral Activity | Sep. 14, 1982 |
| US-4352792 | 3-Alkoxyflavone Antiviral Agents | Oct. 5, 1982 |
| US-4371537 | Sulfur-Substituted Phenoxypyridines Having Antiviral Activity | Feb. 1, 1983 |
| US-4423053 | Derivatives Of 2-Amino-5-(O-Sulphamidophenyl)-1,3,4-Thiadiazol As Antiviral Agents And A | Dec. 27, 1983 |
| | Process For The Preparation Thereof | |
| US-4505929 | Sulfur-Substituted Diphenyl Ethers Having Antiviral Activity | Mar. 19, 1985 |
| US-4526897 | Hypertensive Isoindolin-2-Yl-Aminoimidazolines And Isoindolin-2-Yl-Guanidines | Jul. 2, 1985 |
| US-4558134 | Certain Phenoxy-Pyridine-Carbonitriles Having Antiviral Activity | Dec. 10, 1985 |
| US-4629729 | Endowed With Anti-Viral Activity 2-Alkylamino-4,6-Dihalo Pyrimidines | Dec. 16, 1986 |
| US-4636492 | Inhibition Of Viral Protease Activity By Peptide Halomethyl Ketones | Jan. 13, 1987 |
| US-4652552 | Tetrapeptide Methyl Ketone Inhibitors Of Viral Proteases | Mar. 24, 1987 |
| US-4724233 | Therapeutical Application Of Phosphonylmethoxyalkyl Adenines | Feb. 9, 1988 |
| US-4738984 | Antirhinovirus Agents | Apr. 19, 1988 |
| US-4847246 | Antiviral Compositions Derived From Fireflies And Their Methods Of Use | Jul. 11, 1989 |
| US-4855283 | Novel Pharmaceutically Active N-(2-Aminoacylamido-2-Deoxy-Hexosyl)-Amides, -Carbamates | Aug. 8, 1989 |
| | And -Ureas | |
| US-4885285 | Phosphorus Compounds, Processes For Their Manufacture, And Their Use | Dec. 5, 1989 |
| US-4956351 | Antiviral Pharmaceutical Compositions Containing Cyclodextrins | Sep. 11, 1990 |
| US-5001125 | Anti-Virally Active Pyridazinamines | Mar. 19, 1991 |
| US-5036072 | Antiviral Agent | Jul. 30, 1991 |
| US-5070090 | Antipicorpaviral Herterocyclic-Substituted Morpholinyl Alkylphenol Ethers | Dec. 3, 1991 |
| US-5100893 | Antipicornaviral Pyridazinamines | Mar. 31, 1992 |
| US-5112825 | Antirhinoviral Heteroamine-Substituted Pyridazines | May 12, 1992 |
| US-5157035 | Anti-Virally Active Pyridazinaniines | Oct. 20, 1992 |
| US-5240694 | Combined Antiviral And Antimediator Treatment Of Common Colds | Aug. 31, 1993 |
| US-5242924 | Tetrazolyl-(Phenoxy And Phenoxyalkyl)-Piperidinylpyridazines As Antiviral Agents | Sep. 7, 1993 |
| US-5278184 | Synthetic Derivatives Of Pyrrole And Pyrrolidine Suitable For The Therapy Of Infections Caused | Jan. 11, 1994 |
| | By Rhinoviruses | |
| US-5364865 | Phenoxy- And Phenoxyalkyl-Piperidines As Antiviral Agents | Nov. 15, 1994 |
| US-5453433 | Thiadiazoles And Antipicornaviral Compositions | Sep. 26, 1995 |
| US-5492689 | Combined Virustatic Antimediator (COVAM) Treatment Of Common Colds | Feb. 20, 1996 |
| US-5514679 | Therapeutic Phenoxyalklpyridazines And Intermediates Therefor | May 7, 1996 |
| US-5514692 | Substituted Quinoline Derivatives Useful As Antipiconaviral Agents | May 7, 1996 |
| US-5523312 | Antipicornaviral Agents | Jun. 4, 1996 |
| US-5545653 | Anti-Viral Compounds | Aug. 13, 1996 |
| US-5552420 | Therapeutic Phenoxyalkylazoles And Phenoxyalkylazines | Sep. 3, 1996 |
| US-5567719 | Thiadiazoles And Their Use As Antipicornaviral Agents | Oct. 22, 1996 |
| US-5580897 | 1,2-Dithiins Having Antifungal Activity | Dec. 3, 1996 |
| US-5618821 | Therapeutic Phenoxyalkylheterocycles | Apr. 8, 1997 |
| US-5618849 | Orally Active Antiviral Compounds | Apr. 8, 1997 |
| US-5648354 | 1,2-Dithiins Having Antifungal Activity | Jul. 15, 1997 |
| US-5650419 | Thiadiazoles And Their Use As Antipicornaviral Agents | Jul. 22, 1997 |
| US-5693661 | Anti-Viral Compounds | Dec. 2, 1997 |
| US-5721261 | Therapeutic Phenoxyalkylazoles And Phenoxyalkylazines | Feb. 24, 1998 |
| US-5725859 | Plant-Based Therapeutic Agent With Virustatic And Antiviral Effect | Mar. 10, 1998 |
| US-5750527 | Thiadiazoles And Their Use As Antipicornaviral Agents | May 12, 1998 |
| US-5750551 | Treatment For Viral Diseases | May 12, 1998 |
| US-5762940 | Methods And Compositions For Inhibiting Or Destroying Viruses Or Retroviruses | Jun. 9, 1998 |
| US-5763461 | Therapeutic Phenoxyalkylheterocycles | Jun. 9, 1998 |
| US-5821242 | Anti-Viral Compounds | Oct. 13, 1998 |
| US-5821257 | Thiadiazoles And Their Uses As Antipicornaviral Agents | Oct. 13, 1998 |
| US-5821331 | Anti-Picornaviral Agents | Oct. 13, 1998 |
| US-5846986 | Therapeutic Phenoxyalkylazoles And Phenoxyalkylazines | Dec. 8, 1998 |
| US-5856530 | Antipicornaviral Compounds And Methods For Their Use And Preparation | Jan. 5, 1999 |
| US-5891874 | Anti-Viral Compound | Apr. 6, 1999 |
| US-5962487 | Antipicornaviral Compounds And Methods For Their Use And Preparation | Oct. 5, 1999 |
| US-6004933 | Cysteine Protease Inhibitors | Dec. 21, 1999 |
| US-6020371 | Antipicornaviral Compounds Compositions Containing Them And Methods For Their Use | Feb. 1, 2000 |
| US-6087374 | Anti-Viral Compounds | Jul. 11, 2000 |
| US-6114327 | Anti-Viral Compounds | Sep. 5, 2000 |
| US-6117844 | Method And Composition For Antiviral Therapy | Sep. 12, 2000 |
| US-6194447 | Bis (Benzimidazole) Derivatives Serving As Potassium Blocking Agents | Feb. 27, 2001 |
| US-6214799 | Antipicornaviral Compounds And Methods For Their Use And Preparation | Apr. 10, 2001 |
| US-6277891 | Nitric Oxide Inhibits Rhinovirus Infection | Aug. 21, 2001 |
| US-6294186 | Antimicrobial Compositions Comprising A Benzoic Acid Analog And A Metal Salt | Sep. 25, 2001 |
| US-6331554 | Antipicornaviral Compounds, Compositions Containing Them, And Methods For Their Use | Dec. 18, 2001 |
| US-6358971 | Anti-Viral Compounds | Mar. 19, 2002 |
| US-6362166 | Antipicornaviral Compounds And Methods For Their Use And Preparation | Mar. 26, 2002 |
| US-6414004 | 3-Substituted 5-Aryl-4-Isoxazolecarbonitriles Having Antiviral Activity | Jul. 2, 2002 |
| US-6420591 | Carbamates And Compositions Thereof, And Methods For Their Use For Treating Cancer, | Jul. 16, 2002 |
| | Inflammation, Or A Viral Infection | |
| US-6469018 | Compounds | Oct. 22, 2002 |
| US-6498178 | Inhibitors Of IMPDH Enzyme | Dec. 24, 2002 |
| US-6514997 | Antipicornaviral Compounds And Compositions, Their Pharmaceutical Uses, And Materials For | Feb. 4, 2003 |
| | Their Synthesis | |
| US-6525043 | Use Of Ion Channel Modulating Agents | Feb. 25, 2003 |
| US-6531452 | Antipicornaviral Compounds And Compositions, Their Pharmaceutical Uses, And Materials For | Mar. 11, 2003 |
| | Their Synthesis | |
| US-6534489 | Organophosphorus Compounds And The Use Thereof | Mar. 18, 2003 |
| WO 00/06529 | Diketoacid-Derivatives As Inhibitors Of Polymerases | Feb. 10, 2000 |
| WO 00/25791 | Pyridin-4-Yl Or Pyrimidin-4-Yl Substituted Pyrazines | May 11, 2000 |
| WO 00/27423 | Methods And Compositions For Treating Common Cold Symptoms | May 18, 2000 |
| WO 00/34308 | Protein Transduction System And Methods Of Use Thereof | Jun. 15, 2000 |
| WO 00/39348 | Methods And Compositions For Identifying Protease Modulators | Jul. 6, 2000 |
| WO 00/40243 | Novel Compounds | Jul. 13, 2000 |
| WO 00/50037 | Nitrosated And Nitrosylated Proton Pump Inhibitors, Compositions And Methods Of Use | Aug. 31, 2000 |
| WO 00/56331 | Inhibitors Of Impdh Enzyme | Sep. 28, 2000 |
| WO 00/56757 | Immunomodulatory Steroids, In Particular The Hemihydrate Of 16.Alpha.-Bromoepiandrosterone | Sep. 28, 2000 |
| WO 00/66096 | New Indication For Use Of Antiepileptic Agents And Medicines | Nov. 9, 2000 |
| WO 00/78746 | Antiviral Agents | Dec. 28, 2000 |
| WO 01/00199 | Compounds Obtained From Salvia Species Having Antiviral Activity | Jan. 4, 2001 |
| WO 01/00585 | Pyrazolidinol Compounds | Jan. 4, 2001 |
| WO 01/02551 | Virus Like Particles, Their Preparation And Their Use Preferably In Pharmaceutical Screening | Jan. 11, 2001 |
| | And Functional Genomics | |
| WO 01/03681 | Use Of Flavones, Coumarins And Related Compounds To Treat Infections | Jan. 18, 2001 |
| WO 01/05396 | Use Of Cobalt Chelates For Treating Or Preventing Virus Infection | Jan. 25, 2001 |
| WO 01/10894 | Antipicornaviral Compounds And Compositions, Their Pharmaceutical Uses, And Materials For | Feb. 15, 2001 |
| | Their Synthesis | |
| WO 01/19322 | Use Of Csaids In Rhinovirus Infection | Mar. 22, 2001 |
| WO 01/19822 | Antiviral Agents | Mar. 22, 2001 |
| WO 01/22920 | Colon And Colon Cancer Associated Polynucleotides And Polypeptides | Apr. 5, 2001 |
| WO 01/25188 | Novel Carbamates And Ureas | Apr. 12, 2001 |
| WO 01/31016 | Processed Human Chemokines Phc-1 And Phc-2 | May 3, 2001 |
| WO 01/37837 | 3,4-Dihydro-(1h)-Quinazolin-2-Ones And Their Use As Csbp/P38 Kinase Inhibitors | May 31, 2001 |
| WO 01/38312 | 3,4-Dihydro-(1h)Quinazolin-2-One Compounds As Csbp/P38 Kinase Inhibitors | May 31, 2001 |
| WO 01/38313 | 3,4-Dihydro-(1h)Quinazolin-2-One Compounds As Csbp/P39 Kinase Inhibitors | May 31, 2001 |
| WO 01/38314 | 3,4-Dihydro-(1h)Quinazolin-2-One Compounds As Csbp/P38 Kinase Inhibitors | May 31, 2001 |
| WO 01/40189 | Antipicornaviral Compounds And Compositions, Their Pharmaceutical Uses, And Materials For | Jun. 7, 2001 |
| | Their Synthesis | |
| WO 01/49303 | Multivalent Electron Active Compositions And Methods Of Making And Using Same | Jul. 12, 2001 |
| WO 01/60393 | Selective Destruction Of Cells Infected With Human Immunodeficiency Virus | Aug. 23, 2001 |
| WO 01/62726 | 2-Oxo-1-Pyrrolidine Derivatives, Processes For Preparing Them And Their Uses | Aug. 30, 2001 |
| WO 01/79167 | Antipicornaviral Compounds And Compositions, Their Pharmaceutical Uses, And Materials For | Oct. 25, 2001 |
| | Their Synthesis | |
| WO 01/90047 | Novel Mmp-2/Mmp-9 Inhibitors | Nov. 29, 2001 |
| WO 01/90129 | Prophylactic And Therapeutic Treatment Of Infectious And Other Diseases With Mono- And | Nov. 29, 2001 |
| | Disaccharide-Based Compounds | |
| WO 01/92499 | Nucleic Acid Molecules Encoding A Protein Interacting With Ser/Thr Kinase Akt | Dec. 6, 2001 |
| WO 01/93883 | Therapeutic Agents - Iii | Dec. 13, 2001 |
| WO 01/93884 | Therapeutic Agents - I | Dec. 13, 2001 |
| WO 01/93885 | Therapeutic Agents - Ii | Dec. 13, 2001 |
| WO 01/96297 | Antipicornaviral Compounds And Compositions, Their Pharmaceutical Uses, And Materials For | Dec. 20, 2001 |
| | Their Synthesis | |
| WO 02/04413 | Chiral Integrin Modulators And Methods Of Use Thereof | Jan. 17, 2002 |
| WO 02/11743 | Treatment Of Prostate Cancer | Feb. 14, 2002 |
| WO 02/12477 | Enhanced Replication Of Hcv Rna | Feb. 14, 2002 |
| WO 02/14343 | Immunosuppressive, Antiinflammatory And Analgetic Compounds | Feb. 21, 2002 |
| WO 02/24145 | Antiviral Substances From Plant Cuticular And Epicuticular Material | Mar. 28, 2002 |
| WO 02/28351 | Recombinant Mucin Binding Proteins From Steptococcus Pneumoniae | Apr. 11, 2002 |
| WO 02/30442 | Method For Treating Cytokine Mediated Hepatic Injury | Apr. 18, 2002 |
| WO 02/34771 | Nucleic Acids And Proteins From Streptococcus Groups A & B | May 2, 2002 |
| WO 02/44737 | Diagnostic And Therapeutic Compositions And Methods Related To G Protein-Coupled Receptor | Jun. 6, 2002 |
| | (Gpcr) Anaphylatoxin C3a Receptor | |
| WO 02/50045 | Antiviral Agents | Jun. 27, 2002 |
| WO 02/51413 | Macrocyclic Anti-Viral Compounds | Jul. 4, 2002 |
| WO 02/53138 | Treatment For Inhibiting Neoplastic Lesions | Jul. 11, 2002 |
| WO 02/57425 | Nucleoside Derivatives As Inhibitors Of Rna-Dependent Rna Viral Polymerase | Jul. 25, 2002 |
| WO 02/59083 | Novel Compounds | Aug. 1, 2002 |
| WO 02/60875 | Nicotinamide Biaryl Derivatives Useful As Inhibitors Of Pde4 Isozymes | Aug. 8, 2002 |
| WO 02/60898 | Thiazolyl-, Oxazolyl-, Pyrrolyl-, And Imidazolyl-Acid Amide Derivatives Useful As Inhibitors | Aug. 8, 2002 |
| | Of Pde4 Isozymes | |
| WO 02/69903 | Nucleosides, Preparation Thereof And Use As Inhibitors Of Rna Viral Polymerases | Sep. 12, 2002 |
| WO 02/72022 | Substituted Tetracycline Compounds As Antifungal Agents | Sep. 19, 2002 |
| WO 02/72031 | Substituted Tetracycline Compounds As Synergistic Antifungal Agents | Sep. 19, 2002 |
| WO 02/76939 | Cysteine Protease Inhibitors | Oct. 3, 2002 |
| WO 02/77021 | Streptococcus Pneumoniae Proteins And Nucleic Acids | Oct. 3, 2002 |
| WO 02/79401 | Novel Rgs9 Protein Binding Interactions And Methods Of Use Thereof | Oct. 10, 2002 |
| WO 02/82041 | Production And Use Of Novel Peptide-Based Agents For Use With Bi-Specific Antibodies | Oct. 17, 2002 |
| WO 02/87465 | Compositions And Methods Of Double-Targeting Virus Infections And Cancer Cells | Nov. 7, 2002 |
| WO 02/87500 | Viral Enzyme Activated Prototoxophores And Use Of Same To Treat Viral Infections | Nov. 7, 2002 |
| WO 02/88091 | Inhibitors Of Human Rhinovirus 2a Cysteine Protease | Nov. 7, 2002 |
| WO 02/89832 | Pharmaceutical Compositions For Preventing Or Treating Th1 And Th2 Cell Related Diseases By | Nov. 14, 2002 |
| | Modulating The Th1/Th2 Ratio. | |
| WO 02/92779 | Method For Enriching Tissues In Long Chain Polyunsaturated Fatty Acids | Nov. 21, 2002 |
| WO 02/94185 | Conjugates And Compositions For Cellular Delivery | Nov. 28, 2002 |
| WO 02/94868 | Staphylococcus Aureus Proteins And Nucleic Acids | Nov. 28, 2002 |
| WO 02/96867 | Inhibitors Of Protein Kinase For The Treatment Of Disease | Dec. 5, 2002 |
| WO 02/98424 | Novel Anti-Infectives | Dec. 12, 2002 |
| WO 03/04489 | Compositions And Methods For Inhibiting Prenyltransferases | Jan. 16, 2003 |
| WO 03/08628 | Enzymatic Nucleic Acid Peptide Conjugates | Jan. 30, 2003 |
| WO 03/15744 | Chitin Microparticles And Their Medical Uses | Feb. 27, 2003 |
| WO 03/20222 | Dioxolane And Oxathiolane Derivatives As Inhibitors Of Rna-Dependent Rna Viral Polymerase | Mar. 13, 2003 |
| WO 03/20270 | Oxadiazolyl-Phenoxyalkylisoxazoles, Compositions Thereof And Methods For Their Use As | Mar. 13, 2003 |
| | Anti-Picornaviral Agents | |
| WO 03/20271 | Oxadiazolyl-Phenoxyalkylisoxazoles, Compositions Thereof And Methods For Their Use As | Mar. 13, 2003 |
| | Anti-Picornaviral Agents | |
| WO 03/20712 | Oxadiazolyl-Phenoxyalkylthiadiazoles, Compositions Thereof And Methods For Their Use As | Mar. 13, 2003 |
| | Anti-Picornaviral Agents | |
| WO 86/03412 | Improvements Relating To The Treatment Control And Prevention Of Rhinovirus Infections | Jun. 19, 1986 |
| WO 86/03971 | Antiviral Agents | Jul. 17, 1986 |
| WO 88/09669 | Avirulent Microbes And Uses Therefor | Dec. 15, 1988 |
| WO 92/03475 | Enterovirus Peptides | Mar. 5, 1992 |
| WO 92/22520 | Orally Active Antiviral Compounds | Dec. 23, 1992 |
| WO 92/22570 | Inhibitors Of Picornavirus Proteases | Dec. 23, 1992 |
| WO 94/00012 | Nucleic Acids And Methods Of Use Thereof For Controlling Viral Pathogens | Jan. 6, 1994 |
| WO 95/03821 | Prosaposin And Cytokine-Derived Peptides As Therapeutic Agents | Feb. 9, 1995 |
| WO 95/09175 | Ring-Expanded Nucleosides And Nucleotides | Apr. 6, 1995 |
| WO 95/11992 | Antiviral Compounds | May 4, 1995 |
| WO 95/31198 | Thiadiazoles And Their Use As Antipicornaviral Agents | Nov. 23, 1995 |
| WO 95/31438 | Therapeutic Phenoxyalkylheterocycles | Nov. 23, 1995 |
| WO 95/31439 | Therapeutic Phenoxyalkylpyridazines And Intermediates Therefor | Nov. 23, 1995 |
| WO 95/31452 | Therapeutic Phenoxyalkylazoles And Phenoxyalkylazines | Nov. 23, 1995 |
| WO 95/34595 | Antiviral Dendrimers | Dec. 21, 1995 |
| WO 95/35103 | A Pharmaceutical Composition For The Prevention And/Or Treatment Of Viral Infections And | Dec. 28, 1995 |
| | Optionally Inflammations As Well As A Method For The Treatment Thereof | |
| WO 96/05836 | Methods Of Treating Cold Symptoms Using Pentoxifylline | Feb. 29, 1996 |
| WO 96/05854 | Combination Preparation, Containing Cyclosporin A Or Fk506 Or Rapamycin And A Xanthine | Feb. 29, 1996 |
| | Derivative | |
| WO 96/09822 | Antipicornaviral Agents | Apr. 4, 1996 |
| WO 96/11211 | Selective Inhibition Of Internally Initiated Rna Translation | Apr. 18, 1996 |
| WO 96/22689 | Multiple Component Rna Catalysts And Uses Thereof | Aug. 1, 1996 |
| WO 96/40641 | Sulfonamide Derivatives As Cell Adhesion Modulators | Dec. 19, 1996 |
| WO 97/08553 | Targeting Of Proteins To The Cell Wall Of Gram-Positive Bacteria | Mar. 6, 1997 |
| WO 97/34566 | Electrophilic Ketones For The Treatment Of Herpesvirus Infections | Sep. 25, 1997 |
| WO 97/41137 | Use Of Anthocyanidin And Anthocyanidin Derivatives | Nov. 6, 1997 |
| WO 97/43305 | Inhibitors Of Picornavirus 3c Proteases And Methods For Their Use And Preparation | Nov. 20, 1997 |
| WO 97/47270 | Novel Anti-Viral Compounds | Dec. 18, 1997 |
| WO 98/03572 | Antiviral Linear Polymers | Jan. 29, 1998 |
| WO 98/07745 | Compositions And Methods For Treating Infections Using Analogues Of Indolicidin | Feb. 26, 1998 |
| WO 98/11778 | Antimicrobial Treatment For Herpes Simplex Virus And Other Infectious Diseases | Mar. 26, 1998 |
| WO 98/22495 | Antikinin Compounds And Uses Thereof | May 28, 1998 |
| WO 98/31363 | Anti-Viral Compounds | Jul. 23, 1998 |
| WO 98/31374 | Method Of Treating Rhinoviral Infections | Jul. 23, 1998 |
| WO 98/32427 | Therapeutic Treatment And Prevention Of Infections With A Bioactive Material Encapsulated | Jul. 30, 1998 |
| | Within A Biodegradable-Biocompatible Polymeric Matrix | |
| WO 98/34601 | Method For Inhibiting Intracellular Viral Replication | Aug. 13, 1998 |
| WO 98/42188 | Antimicrobial Prevention And Treatment Of Human Immunedeficiency Virus And Other | Oct. 1, 1998 |
| | Infectious Diseases | |
| WO 98/43950 | Antipicornaviral Compouds, Compositions Containing Them, And Methods For Their Use | Oct. 8, 1998 |
| WO 98/49190 | Substituted Oxadiazole Cysteine Protease Inhibitors | Nov. 5, 1998 |
| WO 98/55120 | Anti-Viral Compounds | Dec. 10, 1998 |
| WO 99/30699 | Modulators Of Cysteine Protease | Jun. 24, 1999 |
| WO 99/31122 | Antipicornaviral Compounds And Methods For Their Use And Preparation | Jun. 24, 1999 |
| WO 99/54317 | Cysteine Protease Inhibitors | Oct. 28, 1999 |
| WO 99/55663 | Inhibitors Of Impdh Enzyme | Nov. 4, 1999 |
| WO 99/57135 | Antipicornaviral Compounds, Their Preparation And Use | Nov. 11, 1999 |
| WO 99/59587 | Anti-Viral Compounds | Nov. 25, 1999 |
| WO 99/61437 | Novel 2-Alkyl Substituted Imidazole Compounds | Dec. 2, 1999 |

TABLE 2
US Patents and Published International Patent Applications

| Publication | | Publication |
|---|---|---|
| Number | Title | Date |
| WO 02/69903 | Nucleosides, Preparation Thereof And Use As Inhibitors Of Rna Viral Polymerases | Sep. 12, 2002 |
| WO 02/48116 | Inhibitors Of Hepatitis C Virus Ns3 Protease | Jun. 20, 2002 |
| WO 02/48157 | Imidazolidinones And Their Related Derivatives As Hepatitis C Virus Ns3 Protease Inhibitors | Jun. 20, 2002 |
| WO 02/61048 | In Vitro System For Replication Of Rna-Dependent Rna Polymerase (Rdrp) Viruses | Aug. 8, 2002 |
| WO 03/02518 | Novel 2,4-Difluorobenzamide Derivatives As Antiviral Agents | Jan. 9, 2003 |
| WO 02/79187 | Methoxy-1,3,5-Triazine Derivatives As Antiviral Agents | Oct. 10, 2002 |
| WO 01/78648 | 6-Methylnicotinamide Derivatives As Antiviral Agents | Oct. 25, 2001 |
| WO 01/12214 | MYCOPHENOLATE MOFETIL IN ASSOCIATION WITH PEG-IFN-.Alpha. | Feb. 22, 2001 |
| WO 02/100415 | 4′-Substituted Nucleosides | Dec. 19, 2002 |
| WO 02/18404 | Nucleoside Derivatives | Mar. 7, 2002 |
| WO 02/94289 | Antiviral Nucleoside Derivatives | Nov. 28, 2002 |
| WO 96/39500 | Oligonucleotides Specific For Hepatitis C Virus | Dec. 12, 1996 |
| WO 03/00713 | Nucleoside Compounds In Hcv | Jan. 3, 2003 |
| WO 01/60381 | Nucleoside Analogs With Carboxamidine-Modified Bicyclic Base | Aug. 23, 2001 |
| WO 02/03997 | Pyrido[2,3-D]Pyrimidine And Pyrimido[4,5-D]Pyrimidine Nucleosides | Jan. 17, 2002 |
| WO 97/26883 | Modulation Of Th1/Th2 Cytokine Expression By Ribavirin3 And Ribavirin3 Analogs In | Jul. 31, 1997 |
| | Activated T-Lymphocytes | |
| WO 03/26589 | Methods And Compositions For Treating Hepatitis C Virus Using 4′-Modified Nucleosides | Apr. 3, 2003 |
| WO 03/26675 | Methods And Compositions For Treating Flaviviruses And Pestiviruses Using 4′-Modified | Apr. 3, 2003 |
| | Nucleoside | |
| WO 97/30067 | Sugar-Modified Gapped Oligonucleotides | Aug. 21, 1997 |
| WO 01/47883 | Fused-Ring Compounds And Use Thereof As Drugs | Jul. 5, 2001 |
| WO 03/00254 | Fused Cyclic Compounds And Medicinal Use Thereof | Jan. 3, 2003 |
| WO 02/100354 | Pyrrolo[2,3-D]Pyrimidine Nucleoside Analogs | Dec. 19, 2002 |
| WO 01/55111 | Biaryl Compounds, Their Preparation And Their Use In Therapy | Aug. 2, 2001 |
| WO 01/16149 | 2-Azapurine Compounds And Their Use | Mar. 8, 2001 |
| WO 01/85770 | Sentinel Virus Ii | Nov. 15, 2001 |
| WO 02/12263 | Nucleic Acid Binding Compounds Containing Pyrazolo[3,4-D]Pyrimidine Analogues Of Purin- | Feb. 14, 2002 |
| | 2,6-Diamine And Their Uses | |
| JP 2001-247550 A2 | Condensed Ring Compound And Its Medicinal Use | Sep. 11, 2001 |
| 6210675 | PT-NANB Hepatitis Polypeptides | Apr. 3, 2001 |
| 6451991 | Sugar-Modified Gapped Oligonucleotides | Sep. 17, 2002 |
| 5830455 | Method Of Treatment Using A Therapeutic Combination Of α-Interferon And Free Radical | Nov. 3, 1998 |
| | Scavengers | |
| 5908621 | Polyethylene Glycol Modified Interferon Therapy | Jun. 1, 1999 |
| 5990276 | Synthetic Inhibitors Of Hepatitis C Virus NS3 Protease | Nov. 23, 1999 |
| 6172046 | Combination Therapy For Eradicating Detectable HCV-RNA In Patients Having Chronic | Jan. 9, 2001 |
| | Hepatitis C Infection | |
| 6177074 | Polyethylene Glycol Modified Interferon Therapy | Jan. 23, 2001 |
| 6326137 | Hepatitis C Virus Protease-Dependent Chimeric Pestivirus | Dec. 4, 2001 |
| 6434489 | Compositions Of Hepatitis C Virus NS5B Polymerase And Methods For Crystallizing Same | Aug. 13, 2002 |
| 6461605 | Continuous Low-Dose Cytokine Infusion Therapy | Oct. 8, 2002 |
| 6472373 | Combination Therapy For Eradicating Detectable HCV-RNA In Antiviral Treatment Naive | Oct. 29, 2002 |
| | Patients Having Chronic Hepatitis C Infection | |
| 6524570 | Polyethylene Glycol Modified Interferon Therapy | Feb. 25, 2003 |
| WO 00/37097 | Ribavirin-Interferon Alfa Induction Hcv Combination Therapy | Jun. 29, 2000 |
| WO 00/37110 | Ribavirin-Pegylated Interferon Alfa Induction Hcv Combination Therapy | Jun. 29, 2000 |
| WO 00/62799 | Hcv Combination Therapy, Containing Ribavirin In Association With Antioxidants | Oct. 26, 2000 |
| WO 01/58929 | Azapeptides Useful In The Treatment Of Hepatitis C | Aug. 16, 2001 |
| WO 02/32414 | Ribavirin-Pegylated Interferon Alfa Hcv Combination Therapy | Apr. 25, 2002 |
| WO 03/24461 | Hcv Combination Therapy | Mar. 27, 2003 |
| WO 93/20835 | Treatment Of Hepatitis With Gm-Csf | Oct. 28, 1993 |
| WO 96/36702 | Soluble, Active Hepatitis C Virus Protease | Nov. 21, 1996 |
| WO 97/16204 | Continuous Low-Dose Cytokine Infusion Therapy | May 9, 1997 |
| WO 97/43310 | Synthetic Inhibitors Of Hepatitis C Virus Ns3 Protease | Nov. 20, 1997 |
| WO 98/48840 | Polyethylene Glycol-Interferon Alpha Conjugates For Therapy Of Infection | Nov. 5, 1998 |
| WO 99/15194 | Combination Therapy For Eradicating Detectable Hcv-Rna In Patients Having Chronic | Apr. 1, 1999 |
| | Hepatitis C Infection | |
| WO 99/59621 | Combination Therapy Comprising Ribavirin And Interferon Alpha In Antiviral Treatment | Nov. 25, 1999 |
| | Naive Patients Having G Chronic Hepatitis C Infection | |
| WO 02/100846 | Compounds And Methods For The Treatment Or Prevention Of Flavivirus Infections | Dec. 19, 2002 |
| WO 02/100851 | Compounds And Methods For The Treatment Or Prevention Of Flavivirus Infections | Dec. 19, 2002 |
| 5241053 | Fused Proteins Comprising Glycoprotein Gd Of HSV-1 And LTB | Aug. 31, 1993 |
| 5556946 | Interleukin-2/Viral Antigen Protein Chimers | Sep. 17, 1996 |
| 6087484 | Enhancement Of Ribozyme Catalytic Activity By A 2′-O-Substituted Facilitator | Jul. 11, 2000 |
| | Oligonucleotide | |
| 5830905 | Compounds, Compositions And Methods For Treatment Of Hepatitis C | Nov. 3, 1998 |
| 6316492 | Methods For Treating Or Preventing Viral Infections And Associated Diseases | Nov. 13, 2001 |
| 6440985 | Methods For Treating Viral Infections | Aug. 27, 2002 |
| WO 00/10573 | Compounds, Compositions And Methods For Treating Or Preventing Viral Infections And | Mar. 2, 2000 |
| | Associated Diseases | |
| WO 00/13708 | Methods For Treating Or Preventing Viral Infections And Associated Diseases | Mar. 16, 2000 |
| WO 00/18231 | Methods For Treating Or Preventing Viral Infections And Associated Diseases | Apr. 6, 2000 |
| WO 99/51781 | Hepatitis C Virus Ns5b Compositions And Methods Of Use Thereof | Oct. 14, 1999 |
| 6323180 | Hepatitis C Inhibitor Tri-Peptides | Nov. 27, 2001 |
| 6143715 | Hepatitis C Inhibitor Peptide Analogues | Nov. 7, 2000 |
| 6329379 | Hepatitis C Inhibitor Tri-Peptides | Dec. 11, 2001 |
| 6329417 | Hepatitis C Inhibitor Tri-Pepitides | Dec. 11, 2001 |
| 6410531 | Hepatitis C Inhibitor Tri-Peptides | Jun. 25, 2002 |
| 6420380 | Hepatitis C Inhibitor Tri-Peptides | Jul. 16, 2002 |
| 6448281 | Viral Polymerase Inhibitors | Sep. 10, 2002 |
| 6479508 | Viral Polymerase Inhibitors | Nov. 12, 2002 |
| 6534523 | Hepatitis C Inhibitor Tri-Peptides | Mar. 18, 2003 |
| WO 00/09543 | Hepatitis C Inhibitor Tri-Peptides | Feb. 24, 2000 |
| WO 00/09558 | Hepatitis C Inhibitor Peptides | Feb. 24, 2000 |
| WO 00/59929 | Macrocyclic Peptides Active Against The Hepatitis C Virus | Oct. 12, 2000 |
| WO 02/04425 | Viral Polymerase Inhibitors | Jan. 17, 2002 |
| WO 02/70739 | Hcv Polymerase Inhibitor Assay | Sep. 12, 2002 |
| WO 03/07945 | Viral Polymerase Inhibitors | Jan. 30, 2003 |
| WO 03/10140 | Viral Polymerase Inhibitors | Feb. 6, 2003 |
| WO 03/10141 | Viral Polymerase Inhibitors | Feb. 6, 2003 |
| WO 99/07734 | Hepatitis C Inhibitor Peptide Analogues | Feb. 18, 1999 |
| WO 01/16379 | Hepatitis C Virus Replication Inhibitors | Mar. 8, 2001 |
| WO 02/07761 | Inhibiting Hepatitis C Virus Processing And Replication | Jan. 31, 2002 |
| WO 02/57287 | Nucleoside Derivatives As Inhibitors Of Rna-Dependent Rna Viral Polymerase | Jul. 25, 2002 |
| WO 02/57425 | Nucleoside Derivatives As Inhibitors Of Rna-Dependent Rna Viral Polymerase | Jul. 25, 2002 |
| WO 02/70651 | Viral Reporter Particles | Sep. 12, 2002 |
| WO 03/20222 | Dioxolane And Oxathiolane Derivatives As Inhibitors Of Rna-Dependent Rna Viral Polymerase | Mar. 13, 2003 |
| PCT/US2003/ | Thiosemicarbazones as Anti-Virals and Immunopotentiators | Jan. 10, 2003 |
| 041493 | | |

TABLE 3
US Patents and published international patent applications relating to inhalation
technology for the delivery of antiviral compounds of the invention.

| Publication | | Publication |
|---|---|---|
| Number | Title | Date |
| 5740794 | Apparatus and methods for dispersing dry powder medicaments | Apr. 21, 1998 |
| 5775320 | Method and device for delivering aerosolized medicaments | Jul. 7, 1998 |
| 5785049 | Method and apparatus for dispersion of dry powder medicaments | Jul. 28, 1998 |
| 5814607 | Pulmonary delivery of active fragments of parathyroid hormone | Sep. 29, 1998 |
| 5826633 | Powder filling systems, apparatus and methods | Oct. 27, 1998 |
| 5458135 | Method and device for delivering aerosolized medicaments | Oct. 17, 1995 |
| 5607915 | Pulmonary delivery of active fragments of parathyroid hormone | Mar. 4, 1997 |
| 5654007 | Methods and system for processing dispersible fine powders | Aug. 5, 1997 |
| 5922354 | Methods and system for processing dispersible fine powders | Jul. 13, 1999 |
| 5928469 | Process for storage of materials | Jul. 27, 1999 |
| 5976574 | Processes for spray drying hydrophobic drugs in organic solvent suspensions | Nov. 2, 1999 |
| 5985248 | processes for spray drying solutions of hydrophobic drugs and compositions thereof | Nov. 16, 1999 |
| 5994314 | Compositions and methods for nucleic acid delivery to the lung | Nov. 30, 1999 |
| 5997848 | Methods and compositions for pulmonary delivery of insulin | Dec. 7, 1999 |
| 6001336 | Processes for spray drying aqueous suspensions of hydrophobic drugs and compositions thereof | Dec. 14, 1999 |
| 6019968 | Dispersible antibody compositions and methods for their preparation and use | Feb. 1, 2000 |
| 6051256 | Dispersible macromolecule compositions and methods for their preparation and use | Apr. 18, 2000 |
| 6071428 | Stable compositions | Jun. 6, 2000 |
| 6077543 | Systems and processes for spray drying hydrophobic drugs with hydrophilic excipients | Jun. 20, 2000 |
| 6080721 | Pulmonary delivery of active fragments of parathyroid hormone | Jun. 27, 2000 |
| 6089228 | Apparatus and methods for dispersing dry powder medicaments | Jul. 18, 2000 |
| 6103270 | Methods and system for processing dispersible fine powders | Aug. 15, 2000 |
| 6123936 | Methods and compositions for the dry powder formulation of interferons | Sep. 26, 2000 |
| 6136346 | Powdered pharmaceutical formulations having improved dispersibility | Oct. 24, 2000 |
| 6138668 | Method and device for delivering aerosolized medicaments | Oct. 31, 2000 |
| 6165463 | Dispersible antibody compositions and methods for their preparation and use | Dec. 26, 2000 |
| 6182712 | Power filling apparatus and methods for their use | Feb. 6, 2001 |
| 6187344 | Powdered pharmaceutical formulations having improved dispersibility | Feb. 13, 2001 |
| 6207135 | Gaseous microparticles for ultrasonic diagnosis and process for their production | Mar. 27, 2001 |
| 6231851 | Methods and compositions for the dry powder formulation of interferons | May 15, 2001 |
| 6257233 | Dry powder dispersing apparatus and methods for their use | Jul. 10, 2001 |
| 6258341 | Stable glassy state powder formulations | Jul. 10, 2001 |
| 6267155 | Powder filling systems, apparatus and methods | Jul. 31, 2001 |
| 6294204 | Method of producing morphologically uniform microcapsules and microcapsules produced by | Sep. 25, 2001 |
| | this method | |
| 6303582 | Compositions and methods for nucleic acid delivery to the lung | Oct. 16, 2001 |
| 6309623 | Stabilized preparations for use in metered dose inhalers | Oct. 30, 2001 |
| 6309671 | Stable glassy state powder formulations | Oct. 30, 2001 |
| 6358530 | Powdered pharmaceutical formulations having improved dispersibility | Mar. 19, 2002 |
| 6365190 | Systems and processes for spray drying hydrophobic drugs with hydrophilic excipients | Apr. 2, 2002 |
| 6372258 | Methods of spray-drying a drug and a hydrophobic amino acid | Apr. 16, 2002 |
| 6423344 | Dispersible macromolecule compositions and methods for their preparation and use | Jul. 23, 2002 |
| 6426210 | Storage of materials | Jul. 30, 2002 |
| 6433040 | Stabilized bioactive preparations and methods of use | Aug. 13, 2002 |
| 6440337 | Method and apparatus for the formation of particles | Aug. 27, 2002 |
| RE37872 | Storage of materials | Oct. 8, 2002 |
| 6479049 | Methods and compositions for the dry powder formulation of interferons | Nov. 12, 2002 |
| 6503411 | Stable compositions | Jan. 7, 2003 |
| 6509006 | Devices compositions and methods for the pulmonary delivery of aerosolized medicaments | Jan. 21, 2003 |
| 6514496 | Dispersible antibody compositions and methods for their preparation and use | Feb. 4, 2003 |
| 6518239 | dry powder compositions having improved dispersivity | Feb. 11, 2003 |
| 6543448 | apparatus and methods for dispersing dry powder medicaments | Apr. 8, 2003 |
| 6546929 | dry powder dispersing apparatus and methods for their use | Apr. 15, 2003 |
| WO 00/15262 | dry powder active agent pulmonary delivery | Mar. 23, 2000 |
| WO 93/00951 | method and device for delivering aerosolized medicaments | Jan. 21, 1993 |
| WO 94/07514 | pulmonary delivery of active fragments of parathyroid hormone | Apr. 14, 1994 |
| WO 95/24183 | methods and compositions for pulmonary delivery of insulin | Sep. 14, 1995 |
| WO 95/31479 | methods and compositions for the dry powder formulation of interferons | Nov. 23, 1995 |
| WO 96/09085 | apparatus and methods for dispersing dry powder medicaments | Mar. 28, 1996 |
| WO 96/32096 | powdered pharmaceutical formulations having improved dispersibility | Oct. 17, 1996 |
| WO 96/32116 | compositions and methods for nucleic acid delivery to the lung | Oct. 17, 1996 |
| WO 96/32149 | pulmonary delivery of aerosolized medicaments | Oct. 17, 1996 |
| WO 96/32152 | pulmonary administration of dry powder alpha 1-antitrypsin | Oct. 17, 1996 |
| WO 96/40068 | methods and system for processing dispersible fine powders | Dec. 19, 1996 |
| WO 97/41031 | powder filling systems, apparatus and methods | Nov. 6, 1997 |
| WO 97/41833 | dispersible macromolecule compositions and methods for their preparation and use | Nov. 13, 1997 |
| WO 98/16205 | stable glassy state powder formulations | Apr. 23, 1998 |
| WO 98/29096 | aerosolized hydrophobic drug | Jul. 9, 1998 |
| WO 98/29098 | processes for spray drying aqueous suspensions of hydrophobic drugs with hydrophilic | Jul. 9, 1998 |
| | excipients and compositions prepared by such processes | |
| WO 98/29140 | processes and compositions for spray drying hydrophobic drugs in organic solvent suspensions | Jul. 9, 1998 |
| | of hydrophilic excipients | |
| WO 98/29141 | processes for spray drying solutions of hydrophobic drugs with hydrophilic excipients and | Jul. 9, 1998 |
| | compositions prepared by such processes | |
| WO 99/19215 | powder filling apparatus and method | Apr. 22, 1999 |
| WO 99/42124 | liquid crystal forms of cyclosporin | Aug. 26, 1999 |
| WO 99/47196 | aerosolized active agent delivery | Sep. 23, 1999 |
| WO 99/62495 | dry powder dispersing apparatus and methods for their use | Dec. 9, 1999 |
| WO 00/21594 | flow resistance modulated aerosolized active agent delivery | Apr. 20, 2000 |
| WO 00/61178 | pulmonary administration of dry powder formulations for treating infertility | Oct. 19, 2000 |
| WO 00/72904 | apparatus and method for dispensing metered amount of aerosolized medication | Dec. 7, 2000 |
| WO 01/00263 | systems and methods for aerosolizing pharmaceutical formulations | Jan. 4, 2001 |
| WO 01/00312 | spray drying process for preparing dry powders | Jan. 4, 2001 |
| WO 01/32144 | dry powder compositions having improved dispersivity | May 10, 2001 |
| WO 01/43529 | receptacles to facilitate the extraction of powders | Jun. 21, 2001 |
| WO 01/43530 | systems and methods for extracting powders from receptacles | Jun. 21, 2001 |
| WO 01/43802 | systems and methods for treating packaged powders | Jun. 21, 2001 |
| WO 01/44764 | systems and methods for non-destructive mass sensing | Jun. 21, 2001 |
| WO 01/87393 | systems, devices and methods for opening receptacles having a powder to be fluidized | Nov. 22, 2001 |
| WO 01/93932 | lockout mechanism for aerosol drug delivery devices | Dec. 13, 2001 |
| WO 02/09669 | apparatus and process to produce particles having a narrow size distribution and particles made | Feb. 7, 2002 |
| | thereby | |
| WO 02/11695 | inhaleable spray dried 4-helix bundle protein powders having minimized aggregation | Feb. 14, 2002 |
| WO 02/49619 | induced phase transition method for the production of microparticles containing hydrophilic | Jun. 27, 2002 |
| | active agents | |
| WO 02/49620 | induced phase transition method for the production of microparticles containing hydrophobic | Jun. 27, 2002 |
| | active agents | |
| WO 02/54868 | pulmonary delivery of polyene antifungal agents | Jul. 18, 2002 |
| WO 02/87542 | novel methods and compositions for delivering macromolecules to or via the respiratory tract | Nov. 7, 2002 |
| WO 02/100548 | centrifuged rotating drum for treating cohesive powders | Dec. 19, 2002 |
| WO 03/00326 | powder aerosolization apparatus and method | Jan. 3, 2003 |
| WO 03/00329 | flow regulator for aerosol drug delivery device and methods | Jan. 3, 2003 |

TABLE 4
Forward and reverse primers for nucleic acid amplification of SARSV

| | | | | For- | | | | | | | | | | | | |
|---|---|---|---|---|---|---|---|---|---|---|---|---|---|---|---|---|
| | Forward | | | ward | | Reverse | | | | | Prim- | | | Prod- | | Opti- |
| Pair | primer | Forward | Forward | Prim- | Forward | primer | Reverse | Reverse | Reverse | Reverse | er | | Prod- | uct | An- | mum |
| Numb- | SEQ | Primer | Primer | er | Primer | SEQ | Primer | Primer | Primer | Primer | Tm | Product | uct | % | neal | Anneal |
| er | ID NO | Start | Stop | Tm | % GC | ID NO | Start | Stop | Tm | % GC | Diff | Length | Tm | GC | Score | Temp |
| 1 | 1021 | 12726 | 12746 | 51.3 | 47.6 | 3521 | 12996 | 12977 | 50.2 | 40 | 1 | 271 | 75 | 42.8 | 26 | 52.6 |
| 2 | 1022 | 12236 | 12256 | 51.2 | 42.9 | 3522 | 12993 | 12975 | 51.4 | 47.4 | 0.2 | 758 | 76.4 | 42.5 | 26 | 54 |
| 3 | 1023 | 12373 | 12391 | 50.8 | 47.4 | 3523 | 12993 | 12975 | 51.4 | 47.4 | 0.6 | 621 | 76.4 | 43 | 26 | 53.8 |
| 4 | 1024 | 12236 | 12256 | 51.2 | 42.9 | 3524 | 12996 | 12977 | 50.2 | 40 | 0.9 | 761 | 76.4 | 42.3 | 26 | 53.6 |
| 5 | 1025 | 12373 | 12391 | 50.8 | 47.4 | 3525 | 12996 | 12977 | 50.2 | 40 | 0.5 | 624 | 76.4 | 42.8 | 26 | 53.6 |
| 6 | 1026 | 12726 | 12746 | 51.3 | 47.6 | 3526 | 12993 | 12975 | 51.4 | 47.4 | 0.1 | 268 | 75.1 | 43.3 | 26 | 53.1 |
| 7 | 1027 | 2671 | 2692 | 52.1 | 40.9 | 3527 | 3185 | 3164 | 51 | 45.5 | 1.2 | 515 | 75.6 | 41.6 | 26 | 53.3 |
| 8 | 1028 | 28942 | 28961 | 50.2 | 45 | 3528 | 29298 | 29280 | 51.4 | 52.6 | 1.2 | 357 | 76.4 | 44.8 | 26 | 53.6 |
| 9 | 1029 | 19801 | 19819 | 53.2 | 52.6 | 3529 | 19922 | 19901 | 51.5 | 45.5 | 1.7 | 122 | 72.2 | 43.4 | 26 | 51.1 |
| 10 | 1030 | 19800 | 19817 | 50.4 | 50 | 3530 | 19921 | 19901 | 50.2 | 47.6 | 0.3 | 122 | 72.2 | 43.4 | 26 | 50.7 |
| 11 | 1031 | 9930 | 9948 | 51.5 | 52.6 | 3531 | 10605 | 10588 | 51.1 | 50 | 0.4 | 676 | 75.8 | 41.3 | 27 | 53.5 |
| 12 | 1032 | 9933 | 9952 | 50.9 | 45 | 3532 | 10605 | 10588 | 51.1 | 50 | 0.2 | 673 | 75.8 | 41.2 | 27 | 53.4 |
| 13 | 1033 | 9930 | 9949 | 52.2 | 50 | 3533 | 10605 | 10588 | 51.1 | 50 | 1.1 | 676 | 75.8 | 41.3 | 27 | 53.5 |
| 14 | 1034 | 9927 | 9945 | 50.8 | 52.6 | 3534 | 10605 | 10588 | 51.1 | 50 | 0.3 | 679 | 75.8 | 41.2 | 28 | 53.4 |
| 15 | 1035 | 3789 | 3806 | 50 | 50 | 3535 | 4445 | 4425 | 50.6 | 42.9 | 0.5 | 657 | 75.5 | 40.5 | 28 | 52.9 |
| 16 | 1036 | 3788 | 3805 | 50 | 50 | 3536 | 4444 | 4424 | 50.6 | 42.9 | 0.5 | 657 | 75.5 | 40.5 | 28 | 52.9 |
| 17 | 1037 | 3795 | 3813 | 52.1 | 52.6 | 3537 | 4445 | 4425 | 50.6 | 42.9 | 1.5 | 651 | 75.5 | 40.6 | 28 | 53.1 |
| 18 | 1038 | 3787 | 3804 | 50 | 50 | 3538 | 4445 | 4425 | 50.6 | 42.9 | 0.5 | 659 | 75.4 | 40.4 | 28 | 52.9 |
| 19 | 1039 | 19801 | 19819 | 53.2 | 52.6 | 3539 | 19921 | 19900 | 51.8 | 45.5 | 1.4 | 121 | 72.3 | 43.8 | 28 | 51.2 |
| 20 | 1040 | 24418 | 24436 | 50 | 47.4 | 3540 | 25182 | 25164 | 51.4 | 47.4 | 1.4 | 765 | 76.1 | 41.7 | 28 | 53.4 |
| 21 | 1041 | 9929 | 9949 | 53.8 | 47.6 | 3541 | 10449 | 10425 | 54.6 | 40 | 0.8 | 521 | 75.4 | 40.9 | 28 | 54 |
| 22 | 1042 | 2671 | 2692 | 52.1 | 40.9 | 3542 | 3186 | 3165 | 50.4 | 40.9 | 1.7 | 516 | 75.6 | 41.5 | 28 | 53.1 |
| 23 | 1043 | 3792 | 3810 | 52.9 | 52.6 | 3543 | 4446 | 4425 | 51.8 | 45.5 | 1.1 | 655 | 75.5 | 40.6 | 28 | 53.5 |
| 24 | 1044 | 9933 | 9952 | 50.9 | 45 | 3544 | 10449 | 10431 | 50.9 | 47.4 | 0.1 | 517 | 75.3 | 40.8 | 28 | 53.1 |
| 25 | 1045 | 3792 | 3810 | 52.9 | 52.6 | 3545 | 4445 | 4424 | 51.3 | 40.9 | 1.6 | 654 | 75.5 | 40.5 | 28 | 53.3 |
| 26 | 1046 | 25782 | 25806 | 53.5 | 40 | 3546 | 26184 | 26164 | 52.4 | 42.9 | 1.1 | 403 | 74.7 | 40.2 | 28 | 53.1 |
| 27 | 1047 | 9927 | 9945 | 50.8 | 52.6 | 3547 | 10449 | 10431 | 50.9 | 47.4 | 0.1 | 523 | 75.4 | 40.9 | 28 | 53.1 |
| 28 | 1048 | 9927 | 9945 | 50.8 | 52.6 | 3548 | 10449 | 10428 | 51.9 | 40.9 | 1.1 | 523 | 75.4 | 40.9 | 28 | 53.1 |
| 29 | 1049 | 3789 | 3806 | 50 | 50 | 3549 | 4444 | 4424 | 50.6 | 42.9 | 0.5 | 656 | 75.5 | 40.5 | 28 | 53 |
| 30 | 1050 | 3795 | 3813 | 52.1 | 52.6 | 3550 | 4444 | 4424 | 50.6 | 42.9 | 1.5 | 650 | 75.5 | 40.6 | 28 | 53.1 |
| 31 | 1051 | 9933 | 9952 | 50.9 | 45 | 3551 | 10449 | 10428 | 51.9 | 40.9 | 1.1 | 517 | 75.3 | 40.8 | 28 | 53.1 |
| 32 | 1052 | 9930 | 9948 | 51.5 | 52.6 | 3552 | 10449 | 10431 | 50.9 | 47.4 | 0.5 | 520 | 75.4 | 41 | 28 | 53.2 |
| 33 | 1053 | 9930 | 9948 | 51.5 | 52.6 | 3553 | 10449 | 10428 | 51.9 | 40.9 | 0.4 | 520 | 75.4 | 41 | 28 | 53.3 |
| 34 | 1054 | 9929 | 9948 | 53.2 | 50 | 3554 | 10449 | 10425 | 54.6 | 40 | 1.4 | 521 | 75.4 | 40.9 | 28 | 53.8 |
| 35 | 1055 | 9931 | 9952 | 53 | 45.5 | 3555 | 10449 | 10425 | 54.6 | 40 | 1.6 | 519 | 75.3 | 40.8 | 28 | 53.7 |
| 36 | 1056 | 3791 | 3808 | 50 | 50 | 3556 | 4445 | 4425 | 50.6 | 42.9 | 0.5 | 655 | 75.5 | 40.5 | 28 | 52.9 |
| 37 | 1057 | 3791 | 3808 | 50 | 50 | 3557 | 4444 | 4424 | 50.6 | 42.9 | 0.5 | 654 | 75.5 | 40.5 | 28 | 53 |
| 38 | 1058 | 9930 | 9949 | 52.2 | 50 | 3558 | 10449 | 10431 | 50.9 | 47.4 | 1.2 | 520 | 75.4 | 41 | 28 | 53.2 |
| 39 | 1059 | 9930 | 9949 | 52.2 | 50 | 3559 | 10449 | 10428 | 51.9 | 40.9 | 0.3 | 520 | 75.4 | 41 | 28 | 53.5 |
| 40 | 1060 | 3788 | 3805 | 50 | 50 | 3560 | 4445 | 4425 | 50.6 | 42.9 | 0.5 | 658 | 75.5 | 40.4 | 28 | 52.9 |
| 41 | 1061 | 19800 | 19817 | 50.4 | 50 | 3561 | 19921 | 19900 | 51.8 | 45.5 | 1.4 | 122 | 72.2 | 43.4 | 28 | 50.8 |
| 42 | 1062 | 3787 | 3804 | 50 | 50 | 3562 | 4444 | 4424 | 50.6 | 42.9 | 0.5 | 658 | 75.5 | 40.4 | 28 | 52.9 |
| 43 | 1063 | 25782 | 25806 | 53.5 | 40 | 3563 | 26183 | 26163 | 51.7 | 42.9 | 1.7 | 402 | 74.7 | 40.3 | 28 | 52.9 |
| 44 | 1064 | 25782 | 25806 | 53.5 | 40 | 3564 | 26183 | 26160 | 54.5 | 41.7 | 1 | 402 | 74.7 | 40.3 | 28 | 53.5 |
| 45 | 1065 | 25782 | 25806 | 53.5 | 40 | 3565 | 26183 | 26159 | 54.9 | 40 | 1.5 | 402 | 74.7 | 40.3 | 28 | 53.5 |
| 46 | 1066 | 2429 | 2447 | 50.2 | 47.4 | 3566 | 3187 | 3166 | 50.3 | 45.5 | 0.1 | 759 | 76.6 | 43 | 29 | 53.8 |
| 47 | 1067 | 2427 | 2445 | 52.1 | 52.6 | 3567 | 3185 | 3164 | 51 | 45.5 | 1.1 | 759 | 76.7 | 43.1 | 29 | 54.1 |
| 48 | 1068 | 2429 | 2447 | 50.2 | 47.4 | 3568 | 3185 | 3164 | 51 | 45.5 | 0.7 | 757 | 76.6 | 42.9 | 29 | 53.8 |
| 49 | 1069 | 19800 | 19817 | 50.4 | 50 | 3569 | 19923 | 19904 | 50.1 | 50 | 0.3 | 124 | 72.3 | 43.5 | 29 | 50.8 |
| 50 | 1070 | 2427 | 2445 | 52.1 | 52.6 | 3570 | 3187 | 3166 | 50.3 | 45.5 | 1.8 | 761 | 76.7 | 43.1 | 29 | 53.9 |
| 51 | 1071 | 29183 | 29204 | 50.4 | 40.9 | 3571 | 29412 | 29393 | 50.3 | 45 | 0 | 230 | 75.3 | 44.8 | 29 | 52.9 |
| 52 | 1072 | 16367 | 16386 | 51.4 | 50 | 3572 | 16780 | 16760 | 51.4 | 42.9 | 0.1 | 414 | 75 | 40.8 | 30 | 53 |
| 53 | 1073 | 11543 | 11562 | 50.4 | 40 | 3573 | 12254 | 12236 | 50.5 | 47.4 | 0.1 | 712 | 76.2 | 42 | 30 | 53.6 |
| 54 | 1074 | 12976 | 12995 | 51.1 | 45 | 3574 | 13547 | 13528 | 50.2 | 45 | 0.9 | 572 | 77.4 | 45.5 | 30 | 54.3 |
| 55 | 1075 | 12040 | 12057 | 50.6 | 50 | 3575 | 12254 | 12236 | 50.5 | 47.4 | 0.1 | 215 | 75.5 | 45.6 | 30 | 53.1 |
| 56 | 1076 | 12976 | 12996 | 51.8 | 42.9 | 3576 | 13544 | 13525 | 52.6 | 55 | 0.8 | 569 | 77.5 | 45.7 | 30 | 54.8 |
| 57 | 1077 | 10141 | 10160 | 51 | 45 | 3577 | 10605 | 10588 | 51.1 | 50 | 0.1 | 465 | 74.9 | 40.2 | 30 | 52.8 |
| 58 | 1078 | 12235 | 12253 | 50.1 | 52.6 | 3578 | 12996 | 12977 | 50.2 | 40 | 0.1 | 762 | 76.4 | 42.4 | 30 | 53.6 |
| 59 | 1079 | 19795 | 19814 | 50.4 | 45 | 3579 | 19921 | 19901 | 50.2 | 47.6 | 0.3 | 127 | 72.3 | 43.3 | 30 | 50.8 |
| 60 | 1080 | 12235 | 12253 | 50.1 | 52.6 | 3580 | 12993 | 12975 | 51.4 | 47.4 | 1.3 | 759 | 76.5 | 42.6 | 30 | 53.7 |
| 61 | 1081 | 12976 | 12994 | 50.3 | 47.4 | 3581 | 13547 | 13528 | 50.2 | 45 | 0.1 | 572 | 77.4 | 45.5 | 30 | 54.3 |
| 62 | 1082 | 12975 | 12994 | 52.1 | 45 | 3582 | 13544 | 13525 | 52.6 | 55 | 0.5 | 570 | 77.4 | 45.6 | 30 | 54.9 |
| 63 | 1083 | 12977 | 12996 | 50.2 | 40 | 3583 | 13547 | 13528 | 50.2 | 45 | 0 | 571 | 77.3 | 45.4 | 30 | 54.3 |
| 64 | 1084 | 11541 | 11561 | 50.9 | 42.9 | 3584 | 12254 | 12236 | 50.5 | 47.4 | 0.3 | 714 | 76.2 | 42 | 30 | 53.6 |
| 65 | 1085 | 28394 | 28411 | 50.3 | 50 | 3585 | 28672 | 28654 | 50.6 | 52.6 | 0.3 | 279 | 78.6 | 51.6 | 30 | 55.2 |
| 66 | 1086 | 9930 | 9948 | 51.5 | 52.6 | 3586 | 10455 | 10434 | 51.1 | 40.9 | 0.3 | 526 | 75.3 | 40.7 | 30 | 53.1 |
| 67 | 1087 | 8220 | 8238 | 51.5 | 47.4 | 3587 | 8929 | 8911 | 53.4 | 52.6 | 1.9 | 710 | 75.4 | 40 | 30 | 53.3 |
| 68 | 1088 | 9930 | 9949 | 52.2 | 50 | 3588 | 10455 | 10435 | 50.5 | 42.9 | 1.7 | 526 | 75.3 | 40.7 | 30 | 52.9 |
| 69 | 1089 | 12236 | 12256 | 51.2 | 42.9 | 3589 | 12412 | 12392 | 50 | 42.9 | 1.2 | 177 | 73 | 41.2 | 30 | 51.2 |
| 70 | 1090 | 9930 | 9949 | 52.2 | 50 | 3590 | 10455 | 10434 | 51.1 | 40.9 | 1.1 | 526 | 75.3 | 40.7 | 30 | 53.1 |
| 71 | 1091 | 9933 | 9952 | 50.9 | 45 | 3591 | 10455 | 10435 | 50.5 | 42.9 | 0.4 | 523 | 75.2 | 40.5 | 30 | 52.9 |
| 72 | 1092 | 12726 | 12746 | 51.3 | 47.6 | 3592 | 13314 | 13297 | 51 | 50 | 0.3 | 589 | 76.6 | 43.6 | 30 | 54 |
| 73 | 1093 | 9933 | 9952 | 50.9 | 45 | 3593 | 10455 | 10434 | 51.1 | 40.9 | 0.3 | 523 | 75.2 | 40.5 | 30 | 53 |
| 74 | 1094 | 16909 | 16928 | 50.8 | 45 | 3594 | 17501 | 17481 | 51.2 | 42.9 | 0.4 | 593 | 75.9 | 41.8 | 30 | 53.5 |
| 75 | 1095 | 12975 | 12993 | 51.4 | 47.4 | 3595 | 13544 | 13525 | 52.6 | 55 | 1.2 | 570 | 77.4 | 45.6 | 30 | 54.7 |
| 76 | 1096 | 2671 | 2692 | 52.1 | 40.9 | 3596 | 3187 | 3166 | 50.3 | 45.5 | 1.8 | 517 | 75.6 | 41.6 | 30 | 53.1 |
| 77 | 1097 | 19800 | 19818 | 52.1 | 52.6 | 3597 | 19921 | 19900 | 51.8 | 45.5 | 0.3 | 122 | 72.2 | 43.4 | 30 | 51.2 |
| 78 | 1098 | 12975 | 12993 | 51.4 | 47.4 | 3598 | 13547 | 13528 | 50.2 | 45 | 1.2 | 573 | 77.3 | 45.4 | 30 | 54.3 |
| 79 | 1099 | 9930 | 9948 | 51.5 | 52.6 | 3599 | 10455 | 10435 | 50.5 | 42.9 | 1 | 526 | 75.3 | 40.7 | 30 | 52.9 |
| 80 | 1100 | 12976 | 12995 | 51.1 | 45 | 3600 | 13544 | 13525 | 52.6 | 55 | 1.5 | 569 | 77.5 | 45.7 | 30 | 54.6 |
| 81 | 1101 | 24635 | 24653 | 50.5 | 52.6 | 3601 | 25182 | 25164 | 51.4 | 47.4 | 0.9 | 548 | 75.1 | 40.1 | 30 | 52.8 |
| 82 | 1102 | 24633 | 24651 | 50.1 | 52.6 | 3602 | 25182 | 25164 | 51.4 | 47.4 | 1.3 | 550 | 75.2 | 40.2 | 30 | 52.7 |
| 83 | 1103 | 24630 | 24648 | 50.8 | 52.6 | 3603 | 25182 | 25164 | 51.4 | 47.4 | 0.6 | 553 | 75.2 | 40.3 | 30 | 53 |
| 84 | 1104 | 28394 | 28412 | 51.1 | 47.4 | 3604 | 28672 | 28654 | 50.6 | 52.6 | 0.5 | 279 | 78.6 | 51.6 | 30 | 55.3 |
| 85 | 1105 | 28395 | 28413 | 50.2 | 42.1 | 3605 | 28672 | 28654 | 50.6 | 52.6 | 0.4 | 278 | 78.6 | 51.4 | 30 | 55.2 |
| 86 | 1106 | 28396 | 28415 | 51.2 | 45 | 3606 | 28672 | 28654 | 50.6 | 52.6 | 0.6 | 277 | 78.6 | 51.6 | 30 | 55.3 |
| 87 | 1107 | 26421 | 26441 | 51.5 | 42.9 | 3607 | 26587 | 26568 | 52.7 | 45 | 1.2 | 167 | 72.3 | 40.1 | 30 | 51.2 |
| 88 | 1108 | 26421 | 26441 | 51.5 | 42.9 | 3608 | 26589 | 26571 | 51.7 | 47.4 | 0.2 | 169 | 72.4 | 40.2 | 30 | 51.2 |
| 89 | 1109 | 26421 | 26441 | 51.5 | 42.9 | 3609 | 26589 | 26572 | 51 | 50 | 0.5 | 169 | 72.4 | 40.2 | 30 | 51.1 |
| 90 | 1110 | 26421 | 26441 | 51.5 | 42.9 | 3610 | 26590 | 26573 | 51.7 | 50 | 0.3 | 170 | 72.3 | 40 | 30 | 51.2 |
| 91 | 1111 | 26040 | 26061 | 56.4 | 54.5 | 3611 | 26589 | 26568 | 55.2 | 45.5 | 1.2 | 550 | 75.1 | 40 | 30 | 54.2 |
| 92 | 1112 | 26039 | 26057 | 52.6 | 52.6 | 3612 | 26183 | 26160 | 54.5 | 41.7 | 1.9 | 145 | 71.9 | 40.7 | 30 | 51.2 |
| 93 | 1113 | 26039 | 26057 | 52.6 | 52.6 | 3613 | 26182 | 26161 | 51.2 | 40.9 | 1.4 | 144 | 71.7 | 40.3 | 30 | 50.7 |
| 94 | 1114 | 26039 | 26057 | 52.6 | 52.6 | 3614 | 26183 | 26163 | 51.7 | 42.9 | 0.9 | 145 | 71.9 | 40.7 | 30 | 51 |
| 95 | 1115 | 8867 | 8887 | 52.3 | 47.6 | 3615 | 9253 | 9235 | 51.6 | 47.4 | 0.7 | 387 | 75.1 | 41.3 | 30 | 53.2 |
| 96 | 1116 | 10247 | 10267 | 50.5 | 47.6 | 3616 | 10605 | 10588 | 51.1 | 50 | 0.6 | 359 | 74.6 | 40.4 | 30 | 52.4 |
| 97 | 1117 | 11540 | 11557 | 50.4 | 50 | 3617 | 12254 | 12236 | 50.5 | 47.4 | 0.1 | 715 | 76.2 | 42.1 | 30 | 53.6 |
| 98 | 1118 | 11541 | 11560 | 50.1 | 45 | 3618 | 12254 | 12236 | 50.5 | 47.4 | 0.4 | 714 | 76.2 | 42 | 30 | 53.5 |
| 99 | 1119 | 8221 | 8240 | 52.4 | 50 | 3619 | 8929 | 8911 | 53.4 | 52.6 | 1 | 709 | 75.4 | 40.1 | 30 | 53.6 |
| 100 | 1120 | 13039 | 13057 | 51.1 | 52.6 | 3620 | 13177 | 13156 | 50.4 | 40.9 | 0.7 | 139 | 73.9 | 46 | 31 | 52 |
| 101 | 1121 | 19801 | 19819 | 53.2 | 52.6 | 3621 | 19917 | 19895 | 52.5 | 43.5 | 0.8 | 117 | 72 | 43.6 | 31 | 51.2 |
| 102 | 1122 | 19709 | 19730 | 51.3 | 40.9 | 3622 | 19921 | 19900 | 51.8 | 45.5 | 0.5 | 213 | 73.9 | 41.8 | 31 | 52.2 |
| 103 | 1123 | 16366 | 16386 | 54.4 | 52.4 | 3623 | 16774 | 16751 | 53.6 | 41.7 | 0.8 | 409 | 75.1 | 41.1 | 31 | 53.8 |
| 104 | 1124 | 3 | 21 | 53.4 | 52.6 | 3624 | 256 | 235 | 52.6 | 45.5 | 0.8 | 254 | 76.1 | 46.1 | 31 | 54.2 |
| 105 | 1125 | 4 | 22 | 52.3 | 52.6 | 3625 | 314 | 296 | 50.6 | 47.4 | 1.7 | 311 | 76.8 | 46.6 | 31 | 54.1 |
| 106 | 1126 | 13039 | 13058 | 51.8 | 50 | 3626 | 13177 | 13156 | 50.4 | 40.9 | 1.5 | 139 | 73.9 | 46 | 31 | 52 |
| 107 | 1127 | 19800 | 19817 | 50.4 | 50 | 3627 | 19916 | 19895 | 50.2 | 40.9 | 0.2 | 117 | 71.7 | 42.7 | 31 | 50.3 |
| 108 | 1128 | 4645 | 4665 | 50.2 | 42.9 | 3628 | 5306 | 5289 | 50.8 | 50 | 0.5 | 662 | 75.6 | 40.8 | 31 | 53.1 |
| 109 | 1129 | 13039 | 13057 | 51.1 | 52.6 | 3629 | 13747 | 13726 | 50.8 | 40.9 | 0.4 | 709 | 76.6 | 43.2 | 31 | 54 |
| 110 | 1130 | 13039 | 13058 | 51.8 | 50 | 3630 | 13747 | 13726 | 50.8 | 40.9 | 1.1 | 709 | 76.6 | 43.2 | 31 | 54 |
| 111 | 1131 | 3 | 21 | 53.4 | 52.6 | 3631 | 253 | 233 | 51.8 | 47.6 | 1.6 | 251 | 76.2 | 46.2 | 31 | 54 |
| 112 | 1132 | 27365 | 27385 | 53.2 | 47.6 | 3632 | 27464 | 27444 | 53 | 42.9 | 0.2 | 100 | 70.8 | 43 | 31 | 50.6 |
| 113 | 1133 | 24418 | 24436 | 50 | 47.4 | 3633 | 24527 | 24508 | 50.5 | 45 | 0.5 | 110 | 71.3 | 42.7 | 31 | 50 |
| 114 | 1134 | 26708 | 26727 | 50 | 45 | 3634 | 27463 | 27446 | 50 | 44.4 | 0 | 756 | 75.9 | 41.1 | 31 | 53.2 |
| 115 | 1135 | 24179 | 24200 | 53.3 | 40.9 | 3635 | 24936 | 24919 | 51.8 | 50 | 1.5 | 758 | 75.8 | 41 | 31 | 53.7 |
| 116 | 1136 | 26708 | 26727 | 50 | 45 | 3636 | 27462 | 27444 | 50.1 | 42.1 | 0.1 | 755 | 75.9 | 41.2 | 31 | 53.2 |
| 117 | 1137 | 26708 | 26731 | 54.2 | 41.7 | 3637 | 27465 | 27446 | 54.6 | 50 | 0.4 | 758 | 75.9 | 41.3 | 31 | 54.5 |
| 118 | 1138 | 27365 | 27384 | 52.6 | 50 | 3638 | 27464 | 27446 | 51.7 | 47.4 | 0.9 | 100 | 70.8 | 43 | 31 | 50.2 |
| 119 | 1139 | 27365 | 27384 | 52.6 | 50 | 3639 | 27464 | 27445 | 52.4 | 45 | 0.2 | 100 | 70.8 | 43 | 31 | 50.4 |
| 120 | 1140 | 27365 | 27384 | 52.6 | 50 | 3640 | 27464 | 27444 | 53 | 42.9 | 0.4 | 100 | 70.8 | 43 | 31 | 50.4 |
| 121 | 1141 | 27367 | 27385 | 51.4 | 52.6 | 3641 | 27571 | 27552 | 50.1 | 40 | 1.3 | 205 | 74.6 | 43.9 | 31 | 52.4 |
| 122 | 1142 | 27367 | 27385 | 51.4 | 52.6 | 3642 | 27567 | 27547 | 51.1 | 42.9 | 0.2 | 201 | 74.7 | 44.3 | 31 | 52.7 |
| 123 | 1143 | 2427 | 2445 | 52.1 | 52.6 | 3643 | 3186 | 3165 | 50.4 | 40.9 | 1.7 | 760 | 76.7 | 43 | 31 | 53.9 |
| 124 | 1144 | 8867 | 8887 | 52.3 | 47.6 | 3644 | 9256 | 9237 | 50.8 | 45 | 1.5 | 390 | 75.1 | 41.3 | 31 | 52.9 |
| 125 | 1145 | 9934 | 9953 | 50.7 | 50 | 3645 | 10605 | 10588 | 51.1 | 50 | 0.4 | 672 | 75.8 | 41.2 | 31 | 53.4 |
| 126 | 1146 | 2429 | 2447 | 50.2 | 47.4 | 3646 | 3186 | 3165 | 50.4 | 40.9 | 0.2 | 758 | 76.6 | 42.9 | 31 | 53.8 |
| 127 | 1147 | 27365 | 27385 | 53.2 | 47.6 | 3647 | 27464 | 27445 | 52.4 | 45 | 0.8 | 100 | 70.8 | 43 | 31 | 50.4 |
| 128 | 1148 | 19994 | 20011 | 50.4 | 50 | 3648 | 20615 | 20597 | 50.6 | 47.4 | 0.2 | 622 | 75.2 | 40 | 31 | 52.9 |
| 129 | 1149 | 9922 | 9941 | 51.3 | 50 | 3649 | 10605 | 10588 | 51.1 | 50 | 0.2 | 684 | 75.8 | 41.2 | 32 | 53.5 |
| 130 | 1150 | 12962 | 12980 | 50.7 | 47.4 | 3650 | 13544 | 13525 | 52.6 | 55 | 1.8 | 583 | 77.5 | 45.6 | 32 | 54.5 |
| 131 | 1151 | 12965 | 12988 | 54 | 41.7 | 3651 | 13544 | 13525 | 52.6 | 55 | 1.5 | 580 | 77.4 | 45.5 | 32 | 55 |
| 132 | 1152 | 13176 | 13197 | 52.7 | 45.5 | 3652 | 13544 | 13525 | 52.6 | 55 | 0.1 | 369 | 77.1 | 46.3 | 32 | 54.8 |
| 133 | 1153 | 28867 | 28886 | 53.2 | 50 | 3653 | 29298 | 29280 | 51.4 | 52.6 | 1.7 | 432 | 76.8 | 45.1 | 32 | 54.3 |
| 134 | 1154 | 24418 | 24439 | 52.9 | 45.5 | 3654 | 25182 | 25164 | 51.4 | 47.4 | 1.5 | 765 | 76.1 | 41.7 | 32 | 53.8 |
| 135 | 1155 | 24420 | 24440 | 50.8 | 42.9 | 3655 | 25182 | 25164 | 51.4 | 47.4 | 0.6 | 763 | 76.1 | 41.5 | 32 | 53.6 |
| 136 | 1156 | 8867 | 8887 | 52.3 | 47.6 | 3656 | 9107 | 9086 | 51.6 | 45.5 | 0.7 | 241 | 74.1 | 41.5 | 32 | 52.5 |
| 137 | 1157 | 1402 | 1422 | 50.2 | 42.9 | 3657 | 2103 | 2083 | 50.6 | 42.9 | 0.4 | 702 | 76.7 | 43.3 | 32 | 53.8 |
| 138 | 1158 | 25782 | 25805 | 52.1 | 41.7 | 3658 | 26183 | 26163 | 51.7 | 42.9 | 0.4 | 402 | 74.7 | 40.3 | 32 | 52.9 |
| 139 | 1159 | 25781 | 25805 | 53.5 | 40 | 3659 | 26183 | 26160 | 54.5 | 41.7 | 1 | 403 | 74.7 | 40.2 | 32 | 53.4 |
| 140 | 1160 | 25781 | 25805 | 53.5 | 40 | 3660 | 26183 | 26159 | 54.9 | 40 | 1.5 | 403 | 74.7 | 40.2 | 32 | 53.4 |
| 141 | 1161 | 2671 | 2692 | 52.1 | 40.9 | 3661 | 3052 | 3033 | 50.3 | 50 | 1.8 | 382 | 74.8 | 40.6 | 32 | 52.5 |
| 142 | 1162 | 12726 | 12746 | 51.3 | 47.6 | 3662 | 13177 | 13156 | 50.4 | 40.9 | 0.9 | 452 | 76.4 | 43.8 | 32 | 53.7 |
| 143 | 1163 | 16909 | 16928 | 50.8 | 45 | 3663 | 17111 | 17090 | 51.1 | 40.9 | 0.3 | 203 | 75 | 44.8 | 32 | 52.8 |
| 144 | 1164 | 12234 | 12252 | 50.6 | 47.4 | 3664 | 12993 | 12975 | 51.4 | 47.4 | 0.8 | 760 | 76.4 | 42.5 | 32 | 53.8 |
| 145 | 1165 | 26039 | 26057 | 52.6 | 52.6 | 3665 | 26828 | 26810 | 52.9 | 52.6 | 0.2 | 790 | 76.4 | 42.4 | 32 | 54.4 |
| 146 | 1166 | 26039 | 26057 | 52.6 | 52.6 | 3666 | 26694 | 26677 | 51.4 | 50 | 1.2 | 656 | 75.7 | 41 | 32 | 53.5 |
| 147 | 1167 | 26039 | 26057 | 52.6 | 52.6 | 3667 | 26692 | 26674 | 51.9 | 52.6 | 0.7 | 654 | 75.7 | 41 | 32 | 53.6 |
| 148 | 1168 | 26039 | 26057 | 52.6 | 52.6 | 3668 | 26691 | 26673 | 51.3 | 47.4 | 1.3 | 653 | 75.6 | 40.9 | 32 | 53.4 |
| 149 | 1169 | 26039 | 26057 | 52.6 | 52.6 | 3669 | 26687 | 26669 | 51.3 | 47.4 | 1.3 | 649 | 75.6 | 40.8 | 32 | 53.4 |
| 150 | 1170 | 26039 | 26057 | 52.6 | 52.6 | 3670 | 26684 | 26666 | 53.4 | 52.6 | 0.8 | 646 | 75.6 | 40.9 | 32 | 53.8 |
| 151 | 1171 | 26039 | 26057 | 52.6 | 52.6 | 3671 | 26683 | 26665 | 52.7 | 52.6 | 0.1 | 645 | 75.6 | 40.9 | 32 | 53.8 |
| 152 | 1172 | 9934 | 9953 | 50.7 | 50 | 3672 | 10449 | 10431 | 50.9 | 47.4 | 0.2 | 516 | 75.4 | 40.9 | 32 | 53.1 |
| 153 | 1173 | 9927 | 9945 | 50.8 | 52.6 | 3673 | 10455 | 10434 | 51.1 | 40.9 | 0.3 | 529 | 75.3 | 40.6 | 32 | 53 |
| 154 | 1174 | 7728 | 7746 | 51.7 | 52.6 | 3674 | 8188 | 8169 | 50.5 | 45 | 1.2 | 461 | 75.6 | 41.9 | 32 | 53.2 |
| 155 | 1175 | 18550 | 18571 | 50.4 | 40.9 | 3675 | 19216 | 19195 | 50.2 | 40.9 | 0.2 | 667 | 75.7 | 41.1 | 32 | 53.2 |
| 156 | 1176 | 19801 | 19819 | 53.2 | 52.6 | 3676 | 19921 | 19899 | 52.4 | 43.5 | 0.8 | 121 | 72.3 | 43.8 | 32 | 51.4 |
| 157 | 1177 | 19709 | 19730 | 51.3 | 40.9 | 3677 | 19923 | 19904 | 50.1 | 50 | 1.2 | 215 | 73.9 | 41.9 | 32 | 51.9 |
| 158 | 1178 | 4639 | 4659 | 51.1 | 47.6 | 3678 | 5306 | 5289 | 50.8 | 50 | 0.3 | 668 | 75.6 | 40.9 | 32 | 53.3 |
| 159 | 1179 | 19794 | 19813 | 50 | 50 | 3679 | 19921 | 19901 | 50.2 | 47.6 | 0.2 | 128 | 72.6 | 43.8 | 32 | 50.9 |
| 160 | 1180 | 12965 | 12985 | 51.2 | 42.9 | 3680 | 13544 | 13525 | 52.6 | 55 | 1.4 | 580 | 77.4 | 45.5 | 32 | 54.6 |
| 161 | 1181 | 9932 | 9953 | 53 | 45.5 | 3681 | 10449 | 10425 | 54.6 | 40 | 1.6 | 518 | 75.3 | 40.7 | 32 | 53.7 |
| 162 | 1182 | 19795 | 19814 | 50.4 | 45 | 3682 | 19921 | 19900 | 51.8 | 45.5 | 1.4 | 127 | 72.3 | 43.3 | 32 | 50.9 |
| 163 | 1183 | 27366 | 27384 | 52.2 | 52.6 | 3683 | 27468 | 27451 | 51.1 | 50 | 1 | 103 | 71.3 | 43.7 | 32 | 50.3 |
| 164 | 1184 | 27366 | 27384 | 52.2 | 52.6 | 3684 | 27467 | 27450 | 52.1 | 50 | 0.1 | 102 | 71.4 | 44.1 | 32 | 50.7 |
| 165 | 1185 | 27366 | 27384 | 52.2 | 52.6 | 3685 | 27466 | 27449 | 51 | 50 | 1.2 | 101 | 71.5 | 44.6 | 32 | 50.4 |
| 166 | 1186 | 25782 | 25805 | 52.1 | 41.7 | 3686 | 26183 | 26164 | 51 | 45 | 1.1 | 402 | 74.7 | 40.3 | 32 | 52.7 |
| 167 | 1187 | 9934 | 9953 | 50.7 | 50 | 3687 | 10449 | 10428 | 51.9 | 40.9 | 1.2 | 516 | 75.4 | 40.9 | 32 | 53.1 |
| 168 | 1188 | 9925 | 9945 | 53.4 | 52.4 | 3688 | 10449 | 10425 | 54.6 | 40 | 1.2 | 525 | 75.4 | 41 | 32 | 53.9 |
| 169 | 1189 | 19800 | 19817 | 50.4 | 50 | 3689 | 19922 | 19902 | 50 | 42.9 | 0.4 | 123 | 72.1 | 43.1 | 32 | 50.6 |
| 170 | 1190 | 8867 | 8887 | 52.3 | 47.6 | 3690 | 9310 | 9291 | 51.2 | 45 | 1.2 | 444 | 75.4 | 41.4 | 32 | 53.2 |
| 171 | 1191 | 27367 | 27385 | 51.4 | 52.6 | 3691 | 27468 | 27451 | 51.1 | 50 | 0.3 | 102 | 71.4 | 44.1 | 32 | 50.4 |
| 172 | 1192 | 27367 | 27385 | 51.4 | 52.6 | 3692 | 27467 | 27450 | 52.1 | 50 | 0.7 | 101 | 71.5 | 44.6 | 32 | 50.6 |
| 173 | 1193 | 2671 | 2692 | 52.1 | 40.9 | 3693 | 3082 | 3058 | 52.3 | 40 | 0.2 | 412 | 74.9 | 40.5 | 32 | 53.2 |
| 174 | 1194 | 9927 | 9945 | 50.8 | 52.6 | 3694 | 10608 | 10589 | 51 | 50 | 0.2 | 682 | 75.8 | 41.2 | 32 | 53.4 |
| 175 | 1195 | 19800 | 19817 | 50.4 | 50 | 3695 | 19920 | 19899 | 50.2 | 40.9 | 0.3 | 121 | 71.9 | 43 | 32 | 50.5 |
| 176 | 1196 | 13177 | 13197 | 50.3 | 42.9 | 3696 | 13547 | 13528 | 50.2 | 45 | 0.1 | 371 | 76.9 | 45.8 | 32 | 54 |
| 177 | 1197 | 28179 | 28200 | 50.8 | 40.9 | 3697 | 28672 | 28654 | 50.6 | 52.6 | 0.3 | 494 | 79.8 | 51.8 | 32 | 56.1 |
| 178 | 1198 | 27367 | 27385 | 51.4 | 52.6 | 3698 | 27466 | 27449 | 51 | 50 | 0.4 | 100 | 71.6 | 45 | 32 | 50.5 |
| 179 | 1199 | 27366 | 27385 | 52.8 | 50 | 3699 | 27465 | 27446 | 54.6 | 50 | 1.7 | 100 | 71.2 | 44 | 32 | 50.8 |
| 180 | 1200 | 19800 | 19818 | 52.1 | 52.6 | 3700 | 19921 | 19901 | 50.2 | 47.6 | 2 | 122 | 72.2 | 43.4 | 32 | 50.7 |
| 181 | 1201 | 9927 | 9945 | 50.8 | 52.6 | 3701 | 10455 | 10435 | 50.5 | 42.9 | 0.3 | 529 | 75.3 | 40.6 | 32 | 52.9 |
| 182 | 1202 | 28868 | 28887 | 50.7 | 45 | 3702 | 29298 | 29280 | 51.4 | 52.6 | 0.7 | 431 | 76.8 | 45 | 32 | 54.1 |
| 183 | 1203 | 28867 | 28887 | 53.7 | 47.6 | 3703 | 29306 | 29288 | 53.5 | 52.6 | 0.3 | 440 | 76.9 | 45.2 | 32 | 55 |
| 184 | 1204 | 28867 | 28887 | 53.7 | 47.6 | 3704 | 29301 | 29282 | 55.3 | 55 | 1.5 | 435 | 76.9 | 45.3 | 32 | 55.1 |
| 185 | 1205 | 28868 | 28888 | 51.4 | 42.9 | 3705 | 29298 | 29280 | 51.4 | 52.6 | 0 | 431 | 76.8 | 45 | 32 | 54.3 |
| 186 | 1206 | 28867 | 28888 | 54.3 | 45.5 | 3706 | 29306 | 29288 | 53.5 | 52.6 | 0.8 | 440 | 76.9 | 45.2 | 32 | 55 |
| 187 | 1207 | 28867 | 28888 | 54.3 | 45.5 | 3707 | 29301 | 29282 | 55.3 | 55 | 1 | 435 | 76.9 | 45.3 | 32 | 55.2 |
| 188 | 1208 | 28870 | 28889 | 50.1 | 40 | 3708 | 29298 | 29280 | 51.4 | 52.6 | 1.3 | 429 | 76.8 | 45 | 32 | 53.9 |
| 189 | 1209 | 28868 | 28889 | 52 | 40.9 | 3709 | 29306 | 29288 | 53.5 | 52.6 | 1.5 | 439 | 76.9 | 45.1 | 32 | 54.5 |
| 190 | 1210 | 28867 | 28889 | 54.8 | 43.5 | 3710 | 29301 | 29282 | 55.3 | 55 | 0.5 | 435 | 76.9 | 45.3 | 32 | 55.4 |
| 191 | 1211 | 28867 | 28890 | 55.2 | 41.7 | 3711 | 29306 | 29288 | 53.5 | 52.6 | 1.7 | 440 | 76.9 | 45.2 | 32 | 55 |
| 192 | 1212 | 28867 | 28890 | 55.2 | 41.7 | 3712 | 29301 | 29282 | 55.3 | 55 | 0.1 | 435 | 76.9 | 45.3 | 32 | 55.5 |
| 193 | 1213 | 28867 | 28890 | 55.2 | 41.7 | 3713 | 29299 | 29280 | 53.9 | 55 | 1.3 | 433 | 76.9 | 45.3 | 32 | 55.1 |
| 194 | 1214 | 12234 | 12252 | 50.6 | 47.4 | 3714 | 12996 | 12977 | 50.2 | 40 | 0.3 | 763 | 76.4 | 42.3 | 32 | 53.6 |
| 195 | 1215 | 28968 | 28988 | 50.9 | 47.6 | 3715 | 29298 | 29280 | 51.4 | 52.6 | 0.6 | 331 | 76.2 | 44.7 | 32 | 53.7 |
| 196 | 1216 | 28968 | 28989 | 51.5 | 45.5 | 3716 | 29298 | 29280 | 51.4 | 52.6 | 0.1 | 331 | 76.2 | 44.7 | 32 | 53.9 |
| 197 | 1217 | 13230 | 13251 | 52.4 | 45.5 | 3717 | 13544 | 13525 | 52.6 | 55 | 0.1 | 315 | 77.2 | 47.3 | 32 | 54.8 |
| 198 | 1218 | 29186 | 29205 | 50.1 | 40 | 3718 | 29298 | 29280 | 51.4 | 52.6 | 1.3 | 113 | 72.8 | 46 | 32 | 51.1 |
| 199 | 1219 | 29195 | 29213 | 51.9 | 52.6 | 3719 | 29306 | 29288 | 53.5 | 52.6 | 1.6 | 112 | 73.6 | 48.2 | 32 | 52.2 |
| 200 | 1220 | 29195 | 29213 | 51.9 | 52.6 | 3720 | 29298 | 29280 | 51.4 | 52.6 | 0.5 | 104 | 73.1 | 48.1 | 32 | 51.7 |
| 201 | 1221 | 29196 | 29214 | 51.1 | 52.6 | 3721 | 29298 | 29280 | 51.4 | 52.6 | 0.3 | 103 | 73.3 | 48.5 | 32 | 51.7 |
| 202 | 1222 | 29195 | 29214 | 52.6 | 50 | 3722 | 29306 | 29288 | 53.5 | 52.6 | 0.9 | 112 | 73.6 | 48.2 | 32 | 52.4 |
| 203 | 1223 | 29196 | 29215 | 51.8 | 50 | 3723 | 29306 | 29288 | 53.5 | 52.6 | 1.6 | 111 | 73.8 | 48.6 | 32 | 52.3 |
| 204 | 1224 | 29196 | 29215 | 51.8 | 50 | 3724 | 29298 | 29280 | 51.4 | 52.6 | 0.4 | 103 | 73.3 | 48.5 | 32 | 51.8 |
| 205 | 1225 | 29197 | 29216 | 50 | 45 | 3725 | 29298 | 29280 | 51.4 | 52.6 | 1.4 | 102 | 73 | 48 | 32 | 51.2 |
| 206 | 1226 | 29196 | 29216 | 52.5 | 47.6 | 3726 | 29306 | 29288 | 53.5 | 52.6 | 1 | 111 | 73.8 | 48.6 | 32 | 52.5 |
| 207 | 1227 | 29195 | 29216 | 53.8 | 45.5 | 3727 | 29301 | 29282 | 55.3 | 55 | 1.5 | 107 | 73.5 | 48.6 | 32 | 52.7 |
| 208 | 1228 | 29254 | 29273 | 53.1 | 50 | 3728 | 29358 | 29339 | 52.8 | 50 | 0.2 | 105 | 73.4 | 48.6 | 32 | 52.3 |
| 209 | 1229 | 29259 | 29278 | 52.6 | 50 | 3729 | 29358 | 29339 | 52.8 | 50 | 0.2 | 100 | 72.4 | 47 | 32 | 51.6 |
| 210 | 1230 | 1402 | 1422 | 50.2 | 42.9 | 3730 | 1773 | 1755 | 51.7 | 52.6 | 1.5 | 372 | 75.8 | 43.3 | 33 | 53.2 |
| 211 | 1231 | 12726 | 12746 | 51.3 | 47.6 | 3731 | 13326 | 13306 | 50.7 | 42.9 | 0.6 | 601 | 76.7 | 43.6 | 33 | 54 |
| 212 | 1232 | 4 | 22 | 52.3 | 52.6 | 3732 | 269 | 251 | 51.1 | 52.6 | 1.2 | 266 | 76.5 | 46.6 | 33 | 54 |
| 213 | 1233 | 19800 | 19817 | 50.4 | 50 | 3733 | 19923 | 19903 | 50.9 | 47.6 | 0.4 | 124 | 72.3 | 43.5 | 33 | 50.9 |
| 214 | 1234 | 2371 | 2389 | 50.3 | 47.4 | 3734 | 3082 | 3058 | 52.3 | 40 | 2 | 712 | 76.7 | 43.3 | 33 | 53.9 |
| 215 | 1235 | 3 | 21 | 53.4 | 52.6 | 3735 | 270 | 251 | 52.9 | 50 | 0.5 | 268 | 76.4 | 46.3 | 33 | 54.4 |
| 216 | 1236 | 9930 | 9949 | 52.2 | 50 | 3736 | 10183 | 10166 | 50.9 | 50 | 1.3 | 254 | 75.3 | 44.1 | 33 | 53.1 |
| 217 | 1237 | 19795 | 19814 | 50.4 | 45 | 3737 | 19923 | 19904 | 50.1 | 50 | 0.3 | 129 | 72.5 | 43.4 | 33 | 50.9 |
| 218 | 1238 | 8867 | 8887 | 52.3 | 47.6 | 3738 | 9365 | 9347 | 53 | 52.6 | 0.7 | 499 | 75.8 | 42.1 | 33 | 53.9 |
| 219 | 1239 | 2371 | 2389 | 50.3 | 47.4 | 3739 | 3055 | 3036 | 50.6 | 50 | 0.3 | 685 | 76.7 | 43.4 | 33 | 53.9 |
| 220 | 1240 | 19709 | 19730 | 51.3 | 40.9 | 3740 | 19921 | 19901 | 50.2 | 47.6 | 1.1 | 213 | 73.9 | 41.8 | 33 | 51.9 |
| 221 | 1241 | 9930 | 9949 | 52.2 | 50 | 3741 | 10183 | 10165 | 51.7 | 47.4 | 0.5 | 254 | 75.3 | 44.1 | 33 | 53.3 |
| 222 | 1242 | 2371 | 2389 | 50.3 | 47.4 | 3742 | 2747 | 2727 | 50 | 42.9 | 0.3 | 377 | 76.9 | 45.9 | 33 | 54 |
| 223 | 1243 | 24921 | 24938 | 50.4 | 50 | 3743 | 25182 | 25164 | 51.4 | 47.4 | 1 | 262 | 74.2 | 41.2 | 33 | 52.2 |
| 224 | 1244 | 18077 | 18099 | 54.4 | 47.8 | 3744 | 18443 | 18424 | 55.9 | 55 | 1.5 | 367 | 75.8 | 43.3 | 33 | 54.5 |
| 225 | 1245 | 25772 | 25793 | 52.4 | 40.9 | 3745 | 26183 | 26164 | 51 | 45 | 1.3 | 412 | 74.8 | 40.3 | 33 | 52.8 |
| 226 | 1246 | 25769 | 25786 | 50.3 | 50 | 3746 | 26183 | 26164 | 51 | 45 | 0.8 | 415 | 74.9 | 40.5 | 33 | 52.6 |
| 227 | 1247 | 25348 | 25366 | 51.2 | 47.4 | 3747 | 25548 | 25531 | 51.1 | 50 | 0.1 | 201 | 74.3 | 43.3 | 33 | 52.4 |
| 228 | 1248 | 12726 | 12746 | 51.3 | 47.6 | 3748 | 13323 | 13304 | 51.1 | 45 | 0.2 | 598 | 76.7 | 43.6 | 33 | 54.1 |
| 229 | 1249 | 8372 | 8390 | 50.7 | 47.4 | 3749 | 8928 | 8911 | 51.9 | 50 | 1.2 | 557 | 75.1 | 40 | 33 | 52.9 |
| 230 | 1250 | 2671 | 2692 | 52.1 | 40.9 | 3750 | 3189 | 3168 | 51 | 45.5 | 1.2 | 519 | 75.7 | 41.6 | 33 | 53.4 |
| 231 | 1251 | 25348 | 25365 | 50.4 | 50 | 3751 | 25548 | 25531 | 51.1 | 50 | 0.7 | 201 | 74.3 | 43.3 | 33 | 52.2 |
| 232 | 1252 | 19801 | 19819 | 53.2 | 52.6 | 3752 | 19923 | 19902 | 51.5 | 45.5 | 1.7 | 123 | 72.4 | 43.9 | 33 | 51.3 |
| 233 | 1253 | 27442 | 27461 | 51.5 | 40 | 3753 | 27546 | 27527 | 51.3 | 50 | 0.2 | 105 | 71.8 | 44.8 | 33 | 50.8 |
| 234 | 1254 | 8867 | 8887 | 52.3 | 47.6 | 3754 | 9312 | 9293 | 50.6 | 45 | 1.8 | 446 | 75.4 | 41.5 | 33 | 53 |
| 235 | 1255 | 2671 | 2692 | 52.1 | 40.9 | 3755 | 3056 | 3038 | 50.8 | 52.6 | 1.3 | 386 | 74.8 | 40.7 | 33 | 52.7 |
| 236 | 1256 | 13231 | 13251 | 50.1 | 42.9 | 3756 | 13547 | 13528 | 50.2 | 45 | 0.2 | 317 | 76.9 | 46.7 | 33 | 54 |
| 237 | 1257 | 9055 | 9079 | 52.8 | 40 | 3757 | 9310 | 9291 | 51.2 | 45 | 1.7 | 256 | 74.4 | 41.8 | 33 | 52.5 |
| 238 | 1258 | 28821 | 28838 | 50.3 | 50 | 3758 | 29298 | 29280 | 51.4 | 52.6 | 1.1 | 478 | 77 | 45.2 | 33 | 54.1 |
| 239 | 1259 | 9055 | 9079 | 52.8 | 40 | 3759 | 9253 | 9235 | 51.6 | 47.4 | 1.2 | 199 | 73.6 | 41.7 | 33 | 52.1 |
| 240 | 1260 | 23840 | 23863 | 55.2 | 45.8 | 3760 | 24050 | 24031 | 56.5 | 55 | 1.4 | 211 | 75 | 44.5 | 33 | 54.1 |
| 241 | 1261 | 18074 | 18093 | 50.3 | 45 | 3761 | 18233 | 18214 | 52 | 50 | 1.7 | 160 | 73.9 | 44.4 | 33 | 51.9 |
| 242 | 1262 | 27366 | 27384 | 52.2 | 52.6 | 3762 | 27674 | 27654 | 51.9 | 42.9 | 0.3 | 309 | 74.3 | 40.5 | 33 | 52.7 |
| 243 | 1263 | 28967 | 28989 | 53.7 | 47.8 | 3763 | 29301 | 29282 | 55.3 | 55 | 1.5 | 335 | 76.4 | 45.1 | 33 | 54.7 |
| 244 | 1264 | 27366 | 27384 | 52.2 | 52.6 | 3764 | 27674 | 27653 | 52.5 | 40.9 | 0.3 | 309 | 74.3 | 40.5 | 33 | 52.8 |
| 245 | 1265 | 28966 | 28988 | 55.3 | 52.2 | 3765 | 29301 | 29282 | 55.3 | 55 | 0.1 | 336 | 76.4 | 45.2 | 33 | 55.2 |
| 246 | 1266 | 18074 | 18094 | 51.1 | 42.9 | 3766 | 18233 | 18214 | 52 | 50 | 1 | 160 | 73.9 | 44.4 | 33 | 52.1 |
| 247 | 1267 | 28965 | 28984 | 52.9 | 55 | 3767 | 29298 | 29280 | 51.4 | 52.6 | 1.5 | 334 | 76.4 | 45.2 | 33 | 54 |
| 248 | 1268 | 18081 | 18099 | 51.2 | 52.6 | 3768 | 18233 | 18215 | 51.3 | 52.6 | 0.11 | 153 | 74 | 45.1 | 33 | 52.2 |
| 249 | 1269 | 18081 | 18099 | 51.2 | 52.6 | 3769 | 18233 | 18214 | 52 | 50 | 0.81 | 1531 | 74 | 45.1 | 33 | 52.2 |
| 250 | 1270 | 18081 | 18099 | 51.2 | 52.6 | 3770 | 18231 | 18210 | 52.2 | 45.5 | 1 | 151 | 73.6 | 44.4 | 33 | 52 |
| 251 | 1271 | 24480 | 245001 | 53.2 | 47.6 | 3771 | 24815 | 24791 | 54.5 | 40 | 1.3 | 336 | 75.6 | 43.2 | 33 | 54 |
| 252 | 1272 | 24481 | 24503 | 52.7 | 43.5 | 3772 | 24815 | 24791 | 54.5 | 40 | 1.8 | 335 | 75.5 | 43 | 33 | 53.8 |
| 253 | 1273 | 27367 | 27385 | 51.4 | 52.6 | 3773 | 27675 | 27656 | 50 | 40 | 1.4 | 309 | 74.3 | 40.5 | 33 | 52.1 |
| 254 | 1274 | 27367 | 27385 | 51.4 | 52.6 | 3774 | 27674 | 27654 | 51.9 | 42.9 | 0.5 | 308 | 74.4 | 40.6 | 33 | 52.6 |
| 255 | 1275 | 27367 | 27385 | 51.4 | 52.6 | 3775 | 27674 | 27653 | 52.5 | 40.9 | 1.1 | 308 | 74.4 | 40.6 | 33 | 52.6 |
| 256 | 1276 | 18081 | 18099 | 51.2 | 52.6 | 3776 | 18223 | 18206 | 51.8 | 50 | 0.6 | 143 | 73.2 | 44.1 | 33 | 51.7 |
| 257 | 1277 | 18080 | 18099 | 53 | 50 | 3777 | 18220 | 18202 | 54.8 | 52.6 | 1.9 | 141 | 73.1 | 44 | 33 | 52.2 |
| 258 | 1278 | 9933 | 9952 | 50.9 | 45 | 3778 | 10670 | 10649 | 51.3 | 40.9 | 0.5 | 738 | 75.7 | 40.8 | 33 | 53.4 |
| 259 | 1279 | 27665 | 27686 | 51.4 | 40.9 | 3779 | 28208 | 28190 | 51.7 | 52.6 | 0.4 | 544 | 75.1 | 40.1 | 33 | 53.4 |
| 260 | 1280 | 27665 | 27685 | 50.7 | 42.9 | 3780 | 28208 | 28190 | 51.7 | 52.6 | 1.1 | 544 | 75.1 | 40.1 | 33 | 52.9 |
| 261 | 1281 | 27442 | 27461 | 51.5 | 40 | 3781 | 27541 | 27522 | 50.1 | 45 | 1.4 | 100 | 71.2 | 44 | 33 | 50 |
| 262 | 1282 | 28821 | 28840 | 51.8 | 45 | 3782 | 29298 | 29280 | 51.4 | 52.6 | 0.4 | 478 | 77 | 45.2 | 33 | 54.4 |
| 263 | 1283 | 28821 | 28839 | 51.1 | 47.4 | 3783 | 29298 | 29280 | 51.4 | 52.6 | 0.3 | 478 | 77 | 45.2 | 33 | 54.3 |
| 264 | 1284 | 8868 | 8889 | 50.4 | 40.9 | 3784 | 9252 | 9235 | 50.1 | 50 | 0.3 | 385 | 75.1 | 41.3 | 34 | 52.7 |
| 265 | 1285 | 19800 | 19818 | 52.1 | 52.6 | 3785 | 19920 | 19899 | 50.2 | 40.9 | 2 | 121 | 71.9 | 43 | 34 | 50.5 |
| 266 | 1286 | 9055 | 9079 | 52.8 | 40 | 3786 | 9313 | 9293 | 52.1 | 47.6 | 0.7 | 259 | 74.6 | 42.1 | 34 | 52.9 |
| 267 | 1287 | 10142 | 10163 | 51.3 | 40.9 | 3787 | 10605 | 10588 | 51.1 | 50 | 0.2 | 464 | 74.9 | 40.1 | 34 | 52.8 |
| 268 | 1288 | 12726 | 12746 | 51.3 | 47.6 | 3788 | 13312 | 13294 | 51 | 52.6 | 0.3 | 587 | 76.6 | 43.6 | 34 | 54 |
| 269 | 1289 | 9055 | 90791 | 52.8 | 40 | 3789 | 9257 | 9237 | 52.2 | 42.9 | 0.7 | 203 | 73.5 | 41.4 | 34 | 52.2 |
| 270 | 1290 | 7876 | 78951 | 51.5 | 45 | 3790 | 8188 | 8169 | 50.5 | 45 | 1.1 | 313 | 75 | 42.2 | 34 | 52.8 |
| 271 | 1291 | 23843 | 23863 | 50.3 | 42.9 | 3791 | 24527 | 24507 | 51 | 42.9 | 0.7 | 685 | 76 | 41.8 | 34 | 53.4 |
| 272 | 1292 | 10247 | 10267 | 50.5 | 47.6 | 3792 | 10608 | 10589 | 51 | 50 | 0.5 | 362 | 74.6 | 40.3 | 34 | 52.4 |
| 273 | 1293 | 24179 | 24199 | 52.7 | 42.9 | 3793 | 24815 | 24791 | 54.5 | 40 | 1.8 | 637 | 75.8 | 41.3 | 34 | 53.9 |
| 274 | 1294 | 12236 | 12256 | 51.2 | 42.9 | 3794 | 12998 | 12979 | 50.1 | 45 | 1.1 | 763 | 76.4 | 42.5 | 34 | 53.6 |
| 275 | 1295 | 7869 | 7889 | 52.5 | 47.6 | 3795 | 8189 | 8169 | 52 | 47.6 | 0.5 | 321 | 75.3 | 42.7 | 34 | 53.4 |
| 276 | 1296 | 1402 | 1422 | 50.2 | 42.9 | 3796 | 2152 | 2133 | 50.7 | 45 | 0.5 | 751 | 76.7 | 43.1 | 34 | 53.8 |
| 277 | 1297 | 12233 | 12251 | 51.1 | 52.6 | 3797 | 12993 | 12975 | 51.4 | 47.4 | 0.2 | 761 | 76.5 | 42.6 | 34 | 54 |
| 278 | 1298 | 3033 | 3053 | 51.7 | 47.6 | 3798 | 3650 | 3631 | 53.1 | 50 | 1.4 | 618 | 76.4 | 42.9 | 34 | 54.1 |
| 279 | 1299 | 12233 | 12251 | 51.1 | 52.6 | 3799 | 12996 | 12977 | 50.2 | 40 | 0.9 | 764 | 76.4 | 42.4 | 34 | 53.7 |
| 280 | 1300 | 24483 | 24503 | 51 | 42.9 | 3800 | 24938 | 24921 | 50.4 | 50 | 0.6 | 456 | 75.6 | 41.9 | 34 | 53.1 |
| 281 | 1301 | 11541 | 11561 | 50.9 | 42.9 | 3801 | 12253 | 12235 | 50.1 | 52.6 | 0.8 | 713 | 76.2 | 42.1 | 34 | 53.5 |
| 282 | 1302 | 24622 | 24643 | 57.1 | 54.5 | 3802 | 25400 | 25379 | 56 | 50 | 1.1 | 779 | 75.7 | 40.7 | 34 | 54.9 |
| 283 | 1303 | 24622 | 24643 | 57.1 | 54.5 | 3803 | 25400 | 25378 | 56.4 | 47.8 | 0.6 | 779 | 75.7 | 40.7 | 34 | 55 |
| 284 | 1304 | 24630 | 24648 | 50.8 | 52.6 | 3804 | 25403 | 25385 | 51.1 | 47.4 | 0.3 | 774 | 75.7 | 40.6 | 34 | 53.3 |
| 285 | 1305 | 9929 | 9946 | 50 | 50 | 3805 | 10605 | 10588 | 51.1 | 50 | 1 | 677 | 75.8 | 41.2 | 34 | 53.2 |
| 286 | 1306 | 24633 | 24651 | 50.1 | 52.6 | 3806 | 25403 | 25385 | 51.1 | 47.4 | 1 | 771 | 75.6 | 40.5 | 34 | 53.1 |
| 287 | 1307 | 11541 | 11560 | 50.1 | 45 | 3807 | 12253 | 12235 | 50.1 | 52.6 | 0 | 713 | 76.2 | 42.1 | 34 | 53.5 |
| 288 | 1308 | 24635 | 24653 | 50.5 | 52.6 | 3808 | 25403 | 25385 | 51.1 | 47.4 | 0.7 | 769 | 75.6 | 40.4 | 34 | 53.2 |
| 289 | 1309 | 9933 | 9952 | 50.9 | 45 | 3809 | 10608 | 10589 | 51 | 50 | 0.1 | 676 | 75.8 | 41.1 | 34 | 53.4 |
| 290 | 1310 | 24921 | 24938 | 50.4 | 50 | 3810 | 25548 | 25531 | 51.1 | 50 | 0.7 | 628 | 75.6 | 40.9 | 34 | 53.1 |
| 291 | 1311 | 7725 | 7743 | 50.8 | 47.4 | 3811 | 8188 | 8169 | 50.5 | 45 | 0.4 | 464 | 75.6 | 41.8 | 34 | 53.2 |
| 292 | 1312 | 28547 | 28568 | 53.5 | 45.5 | 3812 | 29301 | 29282 | 55.3 | 55 | 1.8 | 755 | 78.5 | 47.5 | 34 | 56.1 |
| 293 | 1313 | 28547 | 28568 | 53.5 | 45.5 | 3813 | 29306 | 29288 | 53.5 | 52.6 | 0 | 760 | 78.5 | 47.5 | 34 | 56.1 |
| 294 | 1314 | 28548 | 28568 | 50.5 | 42.9 | 3814 | 29298 | 29280 | 51.4 | 52.6 | 0.9 | 751 | 78.4 | 47.4 | 34 | 55.2 |
| 295 | 1315 | 28546 | 28567 | 55.1 | 50 | 3815 | 29301 | 29282 | 55.3 | 55 | 0.2 | 756 | 78.5 | 47.6 | 34 | 56.6 |
| 296 | 1316 | 28547 | 28567 | 52.9 | 47.6 | 3816 | 29298 | 29280 | 51.4 | 52.6 | 1.5 | 752 | 78.5 | 47.5 | 34 | 55.5 |
| 297 | 1317 | 28546 | 28565 | 52.2 | 50 | 3817 | 29298 | 29280 | 51.4 | 52.6 | 0.8 | 753 | 78.5 | 47.5 | 34 | 55.5 |
| 298 | 1318 | 28546 | 28565 | 52.2 | 50 | 3818 | 29306 | 29288 | 53.5 | 52.6 | 1.3 | 761 | 78.5 | 47.6 | 34 | 55.7 |
| 299 | 1319 | 28396 | 28416 | 52.4 | 47.6 | 3819 | 28672 | 28654 | 50.6 | 52.6 | 1.8 | 277 | 78.6 | 51.6 | 34 | 55.3 |
| 300 | 1320 | 28396 | 28415 | 51.2 | 45 | 3820 | 28671 | 28652 | 52.8 | 55 | 1.6 | 276 | 78.6 | 51.4 | 34 | 55.4 |
| 301 | 1321 | 28396 | 28415 | 51.2 | 45 | 3821 | 28671 | 28653 | 50.2 | 52.6 | 1 | 276 | 78.6 | 51.4 | 34 | 55.2 |
| 302 | 1322 | 12976 | 12995 | 51.1 | 45 | 3822 | 13545 | 13527 | 50.3 | 52.6 | 0.8 | 570 | 77.4 | 45.6 | 34 | 54.4 |
| 303 | 1323 | 16551 | 16568 | 51.1 | 50 | 3823 | 16711 | 16691 | 51 | 42.9 | 0.1 | 161 | 73.8 | 44.1 | 34 | 52.1 |
| 304 | 1324 | 28395 | 28414 | 51.5 | 45 | 3824 | 28672 | 28654 | 50.6 | 52.6 | 0.9 | 278 | 78.6 | 51.4 | 34 | 55.3 |
| 305 | 1325 | 16555 | 16572 | 50.3 | 50 | 3825 | 16780 | 16760 | 51.4 | 42.9 | 1.1 | 226 | 73.6 | 40.7 | 34 | 51.7 |
| 306 | 1326 | 28394 | 28413 | 51.8 | 45 | 3826 | 28671 | 28652 | 52.8 | 55 | 1 | 278 | 78.6 | 51.4 | 34 | 55.6 |
| 307 | 1327 | 28395 | 28413 | 50.2 | 42.1 | 3827 | 28671 | 28653 | 50.2 | 52.6 | 0 | 277 | 78.5 | 51.3 | 34 | 55.1 |
| 308 | 1328 | 7728 | 7746 | 51.7 | 52.6 | 3828 | 8049 | 8032 | 50.4 | 50 | 1.3 | 322 | 74.9 | 41.6 | 34 | 52.6 |
| 309 | 1329 | 28394 | 28412 | 51.1 | 47.4 | 3829 | 28671 | 28652 | 52.8 | 55 | 1.7 | 278 | 78.6 | 51.4 | 34 | 55.4 |
| 310 | 1330 | 28394 | 28412 | 51.1 | 47.4 | 3830 | 28671 | 28653 | 50.2 | 52.6 | 0.8 | 278 | 78.6 | 51.4 | 34 | 55.2 |
| 311 | 1331 | 11543 | 11562 | 50.4 | 40 | 3831 | 12257 | 12237 | 51.3 | 47.6 | 0.9 | 715 | 76.2 | 42 | 34 | 53.5 |
| 312 | 1332 | 28393 | 28411 | 52.9 | 52.6 | 3832 | 28671 | 28652 | 52.8 | 55 | 0.1 | 279 | 78.6 | 51.6 | 34 | 56 |
| 313 | 1333 | 28394 | 28411 | 50.3 | 50 | 3833 | 28671 | 28653 | 50.2 | 52.6 | 0 | 278 | 78.6 | 51.4 | 34 | 55.2 |
| 314 | 1334 | 4255 | 4276 | 51.7 | 45.5 | 3834 | 4710 | 4691 | 50.2 | 45 | 1.5 | 456 | 75.1 | 40.8 | 34 | 52.8 |
| 315 | 1335 | 12975 | 12994 | 52.1 | 45 | 3835 | 13545 | 13526 | 52.9 | 55 | 0.8 | 571 | 77.4 | 45.5 | 34 | 54.9 |
| 316 | 1336 | 9930 | 9948 | 51.5 | 52.6 | 3836 | 10608 | 10589 | 51 | 50 | 0.5 | 679 | 75.8 | 41.2 | 34 | 53.5 |
| 317 | 1337 | 27665 | 27686 | 51.4 | 40.9 | 3837 | 28411 | 28393 | 52.9 | 52.6 | 1.6 | 747 | 76.8 | 43.5 | 34 | 54.3 |
| 318 | 1338 | 27665 | 27686 | 51.4 | 40.9 | 3838 | 28415 | 28396 | 51.2 | 45 | 0.2 | 751 | 76.8 | 43.4 | 34 | 54.2 |
| 319 | 1339 | 11541 | 11561 | 50.9 | 42.9 | 3839 | 12257 | 12237 | 51.3 | 47.6 | 0.5 | 717 | 76.2 | 42 | 34 | 53.7 |
| 320 | 1340 | 27665 | 27685 | 50.7 | 42.9 | 3840 | 28415 | 28396 | 51.2 | 45 | 0.5 | 751 | 76.8 | 43.4 | 34 | 54.1 |
| 321 | 1341 | 11543 | 11562 | 50.4 | 40 | 3841 | 12253 | 12235 | 50.1 | 52.6 | 0.3 | 711 | 76.2 | 42.1 | 34 | 53.5 |
| 322 | 1342 | 11545 | 11563 | 50.8 | 47.4 | 3842 | 12254 | 12236 | 50.5 | 47.4 | 0.2 | 710 | 76.2 | 42.1 | 34 | 53.6 |
| 323 | 1343 | 27436 | 27455 | 52.7 | 45 | 3843 | 27542 | 27522 | 50.9 | 42.9 | 1.8 | 107 | 72 | 44.9 | 34 | 50.8 |
| 324 | 1344 | 27436 | 27455 | 52.7 | 45 | 3844 | 27546 | 27527 | 51.3 | 50 | 1.3 | 111 | 72.7 | 45.9 | 34 | 51.4 |
| 325 | 1345 | 27389 | 27407 | 50.6 | 47.4 | 3845 | 27541 | 27522 | 50.1 | 45 | 0.5 | 153 | 73.2 | 43.1 | 34 | 51.4 |
| 326 | 1346 | 27389 | 27407 | 50.6 | 47.4 | 3846 | 27546 | 27527 | 51.3 | 50 | 0.7 | 158 | 73.5 | 43.7 | 34 | 51.8 |
| 327 | 1347 | 27369 | 27392 | 57.2 | 50 | 3847 | 27468 | 27446 | 57.7 | 47.8 | 0.5 | 100 | 71.2 | 44 | 34 | 52.1 |
| 328 | 1348 | 27367 | 27389 | 55 | 47.8 | 3848 | 27466 | 27446 | 56.8 | 52.4 | 1.8 | 100 | 71.6 | 45 | 34 | 51.7 |
| 329 | 1349 | 11541 | 11560 | 50.1 | 45 | 3849 | 12257 | 12237 | 51.3 | 47.6 | 1.2 | 717 | 76.2 | 42 | 34 | 53.5 |
| 330 | 1350 | 7725 | 7742 | 50 | 50 | 3850 | 8188 | 8169 | 50.5 | 45 | 0.4 | 464 | 75.6 | 41.8 | 34 | 53 |
| 331 | 1351 | 2223 | 2243 | 50.2 | 42.9 | 3851 | 2672 | 2653 | 51.6 | 50 | 1.4 | 450 | 77 | 45.3 | 34 | 54.1 |
| 332 | 1352 | 9930 | 9949 | 52.2 | 50 | 3852 | 10608 | 10589 | 51 | 50 | 1.2 | 679 | 75.8 | 41.2 | 34 | 53.5 |
| 333 | 1353 | 9934 | 9953 | 50.7 | 50 | 3853 | 10455 | 10435 | 50.5 | 42.9 | 0.3 | 522 | 75.3 | 40.6 | 34 | 52.9 |
| 334 | 1354 | 2223 | 2243 | 50.2 | 42.9 | 3854 | 2672 | 2654 | 50.9 | 52.6 | 0.7 | 450 | 77 | 45.3 | 34 | 54.1 |
| 335 | 1355 | 3797 | 3815 | 50.9 | 47.4 | 3855 | 4445 | 4425 | 50.6 | 42.9 | 0.4 | 649 | 75.4 | 40.4 | 34 | 53.1 |
| 336 | 1356 | 9934 | 9953 | 50.7 | 50 | 3856 | 10455 | 10434 | 51.1 | 40.9 | 0.4 | 522 | 75.3 | 40.6 | 34 | 53 |
| 337 | 1357 | 18074 | 18093 | 50.3 | 45 | 3857 | 18697 | 18679 | 51.9 | 52.6 | 1.5 | 624 | 76.2 | 42.5 | 34 | 53.6 |
| 338 | 1358 | 12976 | 12994 | 50.3 | 47.4 | 3858 | 13545 | 13527 | 50.3 | 52.6 | 0.1 | 570 | 77.4 | 45.6 | 34 | 54.4 |
| 339 | 1359 | 12040 | 12057 | 50.6 | 50 | 3859 | 12498 | 12480 | 50 | 47.4 | 0.6 | 459 | 76.3 | 43.6 | 34 | 53.5 |
| 340 | 1360 | 12040 | 12057 | 50.6 | 50 | 3860 | 12257 | 12237 | 51.3 | 47.6 | 0.7 | 218 | 75.4 | 45.4 | 34 | 53.1 |
| 341 | 1361 | 11540 | 11557 | 50.4 | 50 | 3861 | 12257 | 12237 | 51.3 | 47.6 | 0.9 | 718 | 76.2 | 42.1 | 34 | 53.6 |
| 342 | 1362 | 12975 | 12993 | 51.4 | 47.4 | 3862 | 13545 | 13526 | 52.9 | 55 | 1.5 | 571 | 77.4 | 45.5 | 34 | 54.7 |
| 343 | 1363 | 12975 | 12993 | 51.4 | 47.4 | 3863 | 13545 | 13527 | 50.3 | 52.6 | 1.1 | 571 | 77.4 | 45.5 | 34 | 54.4 |
| 344 | 1364 | 11540 | 11560 | 53.2 | 47.6 | 3864 | 11983 | 11965 | 53 | 52.6 | 0.2 | 444 | 75.1 | 40.8 | 34 | 53.6 |
| 345 | 1365 | 12040 | 12057 | 50.6 | 50 | 3865 | 12253 | 12235 | 50.1 | 52.6 | 0.5 | 214 | 75.5 | 45.8 | 34 | 53 |
| 346 | 1366 | 12976 | 12995 | 51.1 | 45 | 3866 | 13545 | 13526 | 52.9 | 55 | 1.8 | 570 | 77.4 | 45.6 | 34 | 54.6 |
| 347 | 1367 | 13039 | 13057 | 51.1 | 52.6 | 3867 | 13314 | 13297 | 51 | 50 | 0.1 | 276 | 75.7 | 44.6 | 34 | 53.4 |
| 348 | 1368 | 27361 | 27380 | 52.4 | 55 | 3868 | 27463 | 27444 | 51.6 | 40 | 0.8 | 103 | 71.7 | 44.7 | 34 | 50.7 |
| 349 | 1369 | 27361 | 27380 | 52.4 | 55 | 3869 | 27463 | 27445 | 50.8 | 42.1 | 1.6 | 103 | 71.7 | 44.7 | 34 | 50.5 |
| 350 | 1370 | 27361 | 27380 | 52.4 | 55 | 3870 | 27464 | 27446 | 51.7 | 47.4 | 0.7 | 104 | 71.9 | 45.2 | 34 | 51 |
| 351 | 1371 | 25348 | 25365 | 50.4 | 50 | 3871 | 25645 | 25626 | 50.8 | 45 | 0.4 | 298 | 74.6 | 41.3 | 34 | 52.4 |
| 352 | 1372 | 9922 | 9941 | 51.3 | 50 | 3872 | 10449 | 10431 | 50.9 | 47.4 | 0.3 | 528 | 75.4 | 40.9 | 34 | 53.2 |
| 353 | 1373 | 13039 | 13057 | 51.1 | 52.6 | 3873 | 13323 | 13304 | 51.1 | 45 | 0 | 285 | 75.8 | 44.6 | 34 | 53.5 |
| 354 | 1374 | 12235 | 12253 | 50.1 | 52.6 | 3874 | 12412 | 12392 | 50 | 42.9 | 0.1 | 178 | 73.2 | 41.6 | 34 | 51.3 |
| 355 | 1375 | 3016 | 3036 | 50.2 | 42.9 | 3875 | 3185 | 3164 | 51 | 45.5 | 0.7 | 170 | 74.5 | 45.3 | 34 | 52.3 |
| 356 | 1376 | 13039 | 13057 | 51.1 | 52.6 | 3876 | 13326 | 13306 | 50.7 | 42.9 | 0.4 | 288 | 75.8 | 44.4 | 34 | 53.4 |
| 357 | 1377 | 7869 | 7889 | 52.5 | 47.6 | 3877 | 8050 | 8032 | 52 | 52.6 | 0.5 | 182 | 73.8 | 42.9 | 34 | 52.3 |
| 358 | 1378 | 26421 | 26441 | 51.5 | 42.9 | 3878 | 26655 | 26634 | 50.6 | 40.9 | 0.9 | 235 | 74.1 | 41.7 | 34 | 52.2 |
| 359 | 1379 | 26421 | 26441 | 51.5 | 42.9 | 3879 | 26657 | 26639 | 50.8 | 47.4 | 0.7 | 237 | 74.2 | 41.8 | 34 | 52.3 |
| 360 | 1380 | 26040 | 26061 | 56.4 | 54.5 | 3880 | 26183 | 26159 | 54.9 | 40 | 1.5 | 144 | 72 | 41 | 34 | 52 |
| 361 | 1381 | 26040 | 26061 | 56.4 | 54.5 | 3881 | 26183 | 26160 | 54.5 | 41.7 | 2 | 144 | 72 | 41 | 34 | 51.9 |
| 362 | 1382 | 26040 | 26061 | 56.4 | 54.5 | 3882 | 26184 | 26161 | 55.1 | 41.7 | 1.3 | 145 | 71.9 | 40.7 | 34 | 52 |
| 363 | 1383 | 12373 | 12391 | 50.8 | 47.4 | 3883 | 12724 | 12705 | 52.4 | 55 | 1.6 | 352 | 75.6 | 42.9 | 34 | 53.2 |
| 364 | 1384 | 26040 | 26061 | 56.4 | 54.5 | 3884 | 26589 | 26569 | 54.7 | 47.6 | 1.7 | 550 | 75.1 | 40 | 34 | 54.1 |
| 365 | 1385 | 26039 | 26058 | 54 | 55 | 3885 | 26183 | 26159 | 54.9 | 40 | 0.9 | 145 | 71.9 | 40.7 | 34 | 51.7 |
| 366 | 1386 | 26039 | 26058 | 54 | 55 | 3886 | 26183 | 26160 | 54.5 | 41.7 | 0.4 | 145 | 71.9 | 40.7 | 34 | 51.7 |
| 367 | 1387 | 26039 | 26058 | 54 | 55 | 3887 | 26183 | 26161 | 54 | 43.5 | 0 | 145 | 71.9 | 40.7 | 34 | 51.7 |
| 368 | 1388 | 26039 | 26058 | 54 | 55 | 3888 | 26184 | 26163 | 53 | 40.9 | 1 | 146 | 71.8 | 40.4 | 34 | 51.3 |
| 369 | 1389 | 26039 | 26057 | 52.6 | 52.6 | 3889 | 26174 | 26153 | 51 | 40.9 | 1.6 | 136 | 71.8 | 41.2 | 34 | 50.7 |
| 370 | 1390 | 10246 | 10266 | 50.4 | 47.6 | 3890 | 10605 | 10588 | 51.1 | 50 | 0.6 | 360 | 74.5 | 40.3 | 34 | 52.4 |
| 371 | 1391 | 3234 | 3254 | 51.1 | 47.6 | 3891 | 3497 | 3478 | 51.3 | 50 | 0.2 | 264 | 74.3 | 41.3 | 34 | 52.4 |
| 372 | 1392 | 26039 | 26057 | 52.6 | 52.6 | 3892 | 26183 | 26162 | 52.81 | 45.5 | 0.2 | 145 | 71.9 | 40.7 | 34 | 51.2 |
| 373 | 1393 | 11540 | 11557 | 50.4 | 50 | 3893 | 12253 | 12235 | 50.1 | 52.6 | 0.3 | 714 | 76.2 | 42.2 | 34 | 53.5 |
| 374 | 1394 | 3234 | 3254 | 51.1 | 47.6 | 3894 | 3500 | 3481 | 51.2 | 50 | 0.1 | 267 | 74.3 | 41.2 | 34 | 52.4 |
| 375 | 1395 | 3794 | 3812 | 52.9 | 52.6 | 3895 | 4445 | 4424 | 51.3 | 40.9 | 1.6 | 652 | 75.5 | 40.5 | 34 | 53.3 |
| 376 | 1396 | 3794 | 3812 | 52.9 | 52.6 | 3896 | 4446 | 4425 | 51.8 | 45.5 | 1.1 | 653 | 75.5 | 40.6 | 34 | 53.5 |
| 377 | 1397 | 3234 | 3254 | 51.1 | 47.6 | 3897 | 3646 | 3625 | 52 | 40.9 | 1 | 413 | 75.1 | 41.2 | 34 | 53 |
| 378 | 1398 | 3234 | 3254 | 51.1 | 47.6 | 3898 | 3647 | 3628 | 50.6 | 45 | 0.5 | 414 | 75.2 | 41.3 | 34 | 52.9 |
| 379 | 1399 | 3226 | 3245 | 51.7 | 55 | 3899 | 3497 | 3478 | 51.3 | 50 | 0.4 | 272 | 74.6 | 41.9 | 34 | 52.7 |
| 380 | 1400 | 3797 | 3815 | 50.9 | 47.4 | 3900 | 4444 | 4424 | 50.6 | 42.9 | 0.4 | 648 | 75.4 | 40.4 | 34 | 53.1 |
| 381 | 1401 | 3226 | 3245 | 51.7 | 55 | 3901 | 3500 | 3481 | 51.2 | 50 | 0.5 | 275 | 74.6 | 41.8 | 34 | 52.7 |
| 382 | 1402 | 16366 | 16384 | 50.3 | 52.6 | 3902 | 16780 | 16760 | 51.4 | 42.9 | 1.1 | 415 | 75.1 | 41 | 34 | 52.7 |
| 383 | 1403 | 25782 | 25806 | 53.5 | 40 | 3903 | 26183 | 26161 | 54 | 43.5 | 0.5 | 402 | 74.7 | 40.3 | 34 | 53.5 |
| 384 | 1404 | 16366 | 16385 | 52.9 | 55 | 3904 | 16780 | 16760 | 51.4 | 42.9 | 1.4 | 415 | 75.1 | 41 | 34 | 53.1 |
| 385 | 1405 | 16367 | 16386 | 51.4 | 50 | 3905 | 16781 | 16761 | 51.3 | 47.6 | 0.1 | 415 | 75.1 | 41 | 34 | 53 |
| 386 | 1406 | 12236 | 12256 | 51.2 | 42.9 | 3906 | 12992 | 12974 | 51.2 | 52.6 | 0 | 757 | 76.5 | 42.5 | 34 | 54 |
| 387 | 1407 | 16367 | 16386 | 51.4 | 50 | 3907 | 16777 | 16758 | 51.5 | 50 | 0.1 | 411 | 75 | 40.9 | 34 | 53 |
| 388 | 1408 | 16367 | 16386 | 51.4 | 50 | 3908 | 16711 | 16691 | 51 | 42.9 | 0.3 | 345 | 75.2 | 42 | 34 | 53 |
| 389 | 1409 | 3226 | 3245 | 51.7 | 55 | 3909 | 3503 | 3484 | 51.5 | 50 | 0.3 | 278 | 74.7 | 42.1 | 34 | 52.9 |
| 390 | 1410 | 16548 | 16566 | 54.9 | 52.6 | 3910 | 16782 | 16760 | 54.3 | 43.5 | 0.6 | 235 | 74 | 41.3 | 34 | 53.2 |
| 391 | 1411 | 16549 | 16567 | 54.9 | 52.6 | 3911 | 16782 | 16760 | 54.3 | 43.5 | 0.6 | 234 | 74 | 41.5 | 34 | 53.2 |
| 392 | 1412 | 25354 | 25372 | 50.9 | 52.6 | 3912 | 25645 | 25626 | 50.8 | 45 | 0.2 | 292 | 74.4 | 41.1 | 34 | 52.4 |
| 393 | 1413 | 16551 | 16568 | 51.1 | 50 | 3913 | 17038 | 17021 | 50.7 | 50 | 0.4 | 488 | 75.8 | 42.2 | 34 | 53.4 |
| 394 | 1414 | 25348 | 25366 | 51.2 | 47.4 | 3914 | 25645 | 25626 | 50.8 | 45 | 0.4 | 298 | 74.6 | 41.3 | 34 | 52.5 |
| 395 | 1415 | 16551 | 16568 | 51.1 | 50 | 3915 | 16780 | 16760 | 51.4 | 42.9 | 0.3 | 230 | 73.9 | 41.3 | 34 | 52.2 |
| 396 | 1416 | 7725 | 7743 | 50.8 | 47.4 | 3916 | 8049 | 8032 | 50.4 | 50 | 0.5 | 325 | 74.9 | 41.5 | 34 | 52.6 |
| 397 | 1417 | 29200 | 29224 | 54.2 | 40 | 3917 | 29299 | 29280 | 53.9 | 55 | 0.3 | 100 | 72.8 | 48 | 34 | 52.3 |
| 398 | 1418 | 29200 | 29224 | 54.2 | 40 | 3918 | 29301 | 29282 | 55.3 | 55 | 1.1 | 102 | 73 | 48 | 34 | 52.4 |
| 399 | 1419 | 29200 | 29223 | 53.7 | 41.7 | 3919 | 29299 | 29280 | 53.9 | 55 | 0.2 | 100 | 72.8 | 48 | 34 | 52.2 |
| 400 | 1420 | 29200 | 29223 | 53.7 | 41.7 | 3920 | 29301 | 29282 | 55.3 | 55 | 1.6 | 102 | 73 | 48 | 34 | 52.3 |
| 401 | 1421 | 29199 | 29222 | 54.6 | 41.7 | 3921 | 29301 | 29282 | 55.3 | 55 | 0.7 | 103 | 72.9 | 47.6 | 34 | 52.5 |
| 402 | 1422 | 29200 | 29222 | 53.2 | 43.5 | 3922 | 29299 | 29280 | 53.9 | 55 | 0.7 | 100 | 72.8 | 48 | 34 | 52 |
| 403 | 1423 | 29199 | 29221 | 54.1 | 43.5 | 3923 | 29301 | 29282 | 55.3 | 55 | 1.2 | 103 | 72.9 | 47.6 | 34 | 52.3 |
| 404 | 1424 | 29200 | 29221 | 52.6 | 45.5 | 3924 | 29299 | 29280 | 53.9 | 55 | 1.3 | 100 | 72.8 | 48 | 34 | 51.9 |
| 405 | 1425 | 18074 | 18093 | 50.3 | 45 | 3925 | 18239 | 18220 | 50 | 45 | 0.3 | 166 | 73.9 | 44 | 34 | 51.8 |
| 406 | 1426 | 18074 | 18093 | 50.3 | 45 | 3926 | 18238 | 18219 | 50.3 | 45 | 0 | 165 | 74 | 44.2 | 34 | 51.9 |
| 407 | 1427 | 1402 | 1426 | 54.1 | 40 | 3927 | 1774 | 1755 | 53.1 | 50 | 1 | 373 | 75.8 | 43.2 | 34 | 54.1 |
| 408 | 1428 | 18074 | 18094 | 51.1 | 42.9 | 3928 | 18697 | 18679 | 51.9 | 52.6 | 0.8 | 624 | 76.2 | 42.5 | 34 | 53.8 |
| 409 | 1429 | 18074 | 18094 | 51.1 | 42.9 | 3929 | 18239 | 18220 | 50 | 45 | 1 | 166 | 73.9 | 44 | 34 | 51.8 |
| 410 | 1430 | 18074 | 18094 | 51.1 | 42.9 | 3930 | 18238 | 18219 | 50.3 | 45 | 0.8 | 165 | 74 | 44.2 | 34 | 51.9 |
| 411 | 1431 | 3226 | 3245 | 51.7 | 55 | 3931 | 3504 | 3485 | 50.4 | 45 | 1.3 | 279 | 74.7 | 41.9 | 34 | 52.5 |
| 412 | 1432 | 18081 | 18099 | 51.2 | 52.6 | 3932 | 18662 | 18641 | 50.4 | 40.9 | 0.7 | 582 | 76.3 | 42.8 | 34 | 53.6 |
| 413 | 1433 | 7725 | 7742 | 50 | 50 | 3933 | 8049 | 8032 | 50.4 | 50 | 0.3 | 325 | 74.9 | 41.5 | 34 | 52.5 |
| 414 | 1434 | 29182 | 29205 | 54.6 | 41.7 | 3934 | 29301 | 29282 | 55.3 | 55 | 0.7 | 120 | 73.4 | 46.7 | 34 | 52.9 |
| 415 | 1435 | 4255 | 4276 | 51.7 | 45.5 | 3935 | 4711 | 4692 | 51.2 | 45 | 0.5 | 457 | 75.1 | 40.7 | 34 | 53 |
| 416 | 1436 | 29183 | 29204 | 50.4 | 40.9 | 3936 | 29298 | 29280 | 51.4 | 52.6 | 1.1 | 116 | 72.8 | 45.7 | 34 | 51.2 |
| 417 | 1437 | 3225 | 3243 | 50.9 | 52.6 | 3937 | 3497 | 3478 | 51.3 | 50 | 0.4 | 273 | 74.7 | 42.1 | 34 | 52.7 |
| 418 | 1438 | 29181 | 29202 | 53.9 | 45.5 | 3938 | 29301 | 29282 | 55.3 | 55 | 1.4 | 121 | 73.6 | 47.1 | 34 | 52.8 |
| 419 | 1439 | 29182 | 29202 | 51.2 | 42.9 | 3939 | 29298 | 29280 | 51.4 | 52.6 | 0.2 | 117 | 73.1 | 46.2 | 34 | 51.6 |
| 420 | 1440 | 29180 | 29199 | 50.1 | 40 | 3940 | 29298 | 29280 | 51.4 | 52.6 | 1.3 | 119 | 73.2 | 46.2 | 34 | 51.4 |
| 421 | 1441 | 28970 | 28993 | 53.3 | 41.7 | 3941 | 29301 | 29282 | 55.3 | 55 | 1.9 | 332 | 76.1 | 44.6 | 34 | 54.4 |
| 422 | 1442 | 28971 | 28993 | 51.9 | 43.5 | 3942 | 29298 | 29280 | 51.4 | 52.6 | 0.5 | 328 | 76.1 | 44.5 | 34 | 53.8 |
| 423 | 1443 | 4255 | 4276 | 51.7 | 45.5 | 3943 | 4711 | 4693 | 50.4 | 47.4 | 1.3 | 457 | 75.1 | 40.7 | 34 | 52.8 |
| 424 | 1444 | 12976 | 12996 | 51.8 | 42.9 | 3944 | 13545 | 13526 | 52.9 | 55 | 1.1 | 570 | 77.4 | 45.6 | 34 | 54.8 |
| 425 | 1445 | 28968 | 28989 | 51.5 | 45.5 | 3945 | 29306 | 29288 | 53.5 | 52.6 | 2 | 339 | 76.3 | 44.8 | 34 | 54 |
| 426 | 1446 | 3225 | 3243 | 50.9 | 52.6 | 3946 | 3500 | 3481 | 51.2 | 50 | 0.3 | 276 | 74.7 | 42 | 34 | 52.6 |
| 427 | 1447 | 3225 | 3243 | 50.9 | 52.6 | 3947 | 3503 | 3484 | 51.5 | 50 | 0.6 | 279 | 74.8 | 42.3 | 34 | 52.7 |
| 428 | 1448 | 3225 | 3243 | 50.9 | 52.6 | 3948 | 3504 | 3485 | 50.4 | 45 | 0.5 | 280 | 74.8 | 42.1 | 34 | 52.6 |
| 429 | 1449 | 28939 | 28961 | 55.2 | 47.8 | 3949 | 29301 | 29282 | 55.3 | 55 | 0.1 | 363 | 76.6 | 45.2 | 34 | 55.3 |
| 430 | 1450 | 28941 | 28961 | 51.6 | 42.9 | 3950 | 29306 | 29288 | 53.5 | 52.6 | 1.8 | 366 | 76.4 | 44.8 | 34 | 54.1 |
| 431 | 1451 | 8867 | 8886 | 50.7 | 50 | 3951 | 9252 | 9235 | 50.1 | 50 | 0.6 | 386 | 75.1 | 41.5 | 34 | 52.7 |
| 432 | 1452 | 28939 | 28960 | 54.7 | 50 | 3952 | 29301 | 29282 | 55.3 | 55 | 0.6 | 363 | 76.6 | 45.2 | 34 | 55.1 |
| 433 | 1453 | 28940 | 28960 | 52.4 | 47.6 | 3953 | 29306 | 29288 | 53.5 | 52.6 | 1 | 367 | 76.5 | 45 | 34 | 54.4 |
| 434 | 1454 | 28941 | 28960 | 50.9 | 45 | 3954 | 29298 | 29280 | 51.4 | 52.6 | 0.5 | 358 | 76.3 | 44.7 | 34 | 53.8 |
| 435 | 1455 | 3360 | 3380 | 51.4 | 42.9 | 3955 | 3494 | 3473 | 50.4 | 40.9 | 1 | 135 | 73.7 | 45.9 | 34 | 51.8 |
| 436 | 1456 | 19709 | 19730 | 51.3 | 40.9 | 3956 | 19916 | 19895 | 50.2 | 40.9 | 1 | 208 | 73.6 | 41.3 | 34 | 51.7 |
| 437 | 1457 | 12373 | 12391 | 50.8 | 47.4 | 3957 | 12994 | 12976 | 50.3 | 47.4 | 0.4 | 622 | 76.4 | 42.9 | 34 | 53.7 |
| 438 | 1458 | 19794 | 19813 | 50 | 50 | 3958 | 19921 | 19900 | 51.8 | 45.5 | 1.8 | 128 | 72.6 | 43.8 | 34 | 50.9 |
| 439 | 1459 | 3361 | 3381 | 50.5 | 42.9 | 3959 | 3494 | 3473 | 50.4 | 40.9 | 0.1 | 134 | 73.8 | 46.3 | 34 | 51.9 |
| 440 | 1460 | 12234 | 12252 | 50.6 | 47.4 | 3960 | 12992 | 12974 | 51.2 | 52.6 | 0.6 | 759 | 76.5 | 42.6 | 34 | 53.8 |
| 441 | 1461 | 12234 | 12252 | 50.6 | 47.4 | 3961 | 12994 | 12976 | 50.3 | 47.4 | 0.2 | 761 | 76.4 | 42.4 | 34 | 53.7 |
| 442 | 1462 | 3034 | 3053 | 50.3 | 50 | 3962 | 3647 | 3628 | 50.6 | 45 | 0.3 | 614 | 76.4 | 42.8 | 34 | 53.6 |
| 443 | 1463 | 8867 | 8887 | 52.3 | 47.6 | 3963 | 9254 | 9236 | 50.6 | 47.4 | 1.7 | 388 | 75.1 | 41.2 | 34 | 52.8 |
| 444 | 1464 | 12726 | 12746 | 51.3 | 47.6 | 3964 | 12994 | 12976 | 50.3 | 47.4 | 1 | 269 | 75.1 | 43.1 | 34 | 52.8 |
| 445 | 1465 | 12234 | 12252 | 50.6 | 47.4 | 3965 | 12998 | 12979 | 50.1 | 45 | 0.5 | 765 | 76.4 | 42.5 | 34 | 53.6 |
| 446 | 1466 | 9926 | 9944 | 50.5 | 52.6 | 3966 | 10605 | 10588 | 51.1 | 50 | 0.6 | 680 | 75.8 | 41.2 | 34 | 53.3 |
| 447 | 1467 | 3034 | 3053 | 50.3 | 50 | 3967 | 3646 | 3625 | 52 | 40.9 | 1.7 | 613 | 76.3 | 42.7 | 34 | 53.6 |
| 448 | 1468 | 12977 | 12996 | 50.2 | 40 | 3968 | 13545 | 13527 | 50.3 | 52.6 | 0 | 569 | 77.4 | 45.5 | 34 | 54.3 |
| 449 | 1469 | 19799 | 19817 | 52.2 | 52.6 | 3969 | 19909 | 19885 | 52.5 | 40 | 0.3 | 111 | 71.6 | 43.2 | 34 | 50.8 |
| 450 | 1470 | 8867 | 8887 | 52.3 | 47.6 | 3970 | 9246 | 9226 | 50.5 | 42.9 | 1.8 | 380 | 75 | 41.1 | 34 | 52.7 |
| 451 | 1471 | 8867 | 8887 | 52.3 | 47.6 | 3971 | 9342 | 9323 | 52.1 | 50 | 0.3 | 476 | 75.7 | 42 | 35 | 53.7 |
| 452 | 1472 | 10141 | 10160 | 51 | 45 | 3972 | 10608 | 10589 | 51 | 50 | 0 | 468 | 74.9 | 40.2 | 35 | 52.8 |
| 453 | 1473 | 3192 | 3213 | 51.8 | 45.5 | 3973 | 3494 | 3473 | 50.4 | 40.9 | 1.4 | 303 | 74.9 | 41.9 | 35 | 52.6 |
| 454 | 1474 | 3360 | 3379 | 50.7 | 45 | 3974 | 3647 | 3628 | 50.6 | 45 | 0.1 | 288 | 75.5 | 43.8 | 35 | 53.1 |
| 455 | 1475 | 27367 | 27385 | 51.4 | 52.6 | 3975 | 27566 | 27546 | 50.7 | 47.6 | 0.7 | 200 | 74.8 | 44.5 | 35 | 52.7 |
| 456 | 1476 | 10250 | 10274 | 51.6 | 40 | 3976 | 10605 | 10588 | 51.1 | 50 | 0.5 | 356 | 74.6 | 40.4 | 35 | 52.6 |
| 457 | 1477 | 27367 | 27385 | 51.4 | 52.6 | 3977 | 27568 | 27548 | 50.2 | 42.9 | 1.2 | 202 | 74.6 | 44.1 | 35 | 52.4 |
| 458 | 1478 | 27367 | 27385 | 51.4 | 52.6 | 3978 | 27571 | 27551 | 51.4 | 42.9 | 0 | 205 | 74.6 | 43.9 | 35 | 52.7 |
| 459 | 1479 | 8867 | 8887 | 52.3 | 47.6 | 3979 | 9376 | 9355 | 51 | 40.9 | 1.3 | 510 | 75.7 | 41.8 | 35 | 53.4 |
| 460 | 1480 | 27367 | 27385 | 51.4 | 52.6 | 3980 | 27576 | 27555 | 51 | 40.9 | 0.4 | 210 | 74.8 | 44.3 | 35 | 52.8 |
| 461 | 1481 | 27367 | 27385 | 51.4 | 52.6 | 3981 | 27579 | 27558 | 51.1 | 40.9 | 0.3 | 213 | 75 | 44.6 | 35 | 52.9 |
| 462 | 1482 | 18704 | 18724 | 50.8 | 47.6 | 3982 | 19215 | 19194 | 50.2 | 40.9 | 0.5 | 512 | 75.5 | 41.2 | 35 | 53 |
| 463 | 1483 | 18704 | 18724 | 50.8 | 47.6 | 3983 | 19217 | 19196 | 50.2 | 40.9 | 0.5 | 514 | 75.5 | 41.2 | 35 | 53 |
| 464 | 1484 | 18696 | 18715 | 51.7 | 50 | 3984 | 19215 | 19194 | 50.2 | 40.9 | 1.5 | 520 | 75.6 | 41.3 | 35 | 53.1 |
| 465 | 1485 | 27365 | 27384 | 52.6 | 50 | 3985 | 27464 | 27443 | 54 | 45.5 | 1.4 | 100 | 70.8 | 43 | 35 | 50.4 |
| 466 | 1486 | 18696 | 18715 | 51.7 | 50 | 3986 | 19217 | 19196 | 50.2 | 40.9 | 1.5 | 522 | 75.6 | 41.4 | 35 | 53.1 |
| 467 | 1487 | 3361 | 3381 | 50.5 | 42.9 | 3987 | 3646 | 3625 | 52 | 40.9 | 1.5 | 286 | 75.5 | 43.7 | 35 | 53.1 |
| 468 | 1488 | 3361 | 3381 | 50.5 | 42.9 | 3988 | 3647 | 3628 | 50.6 | 45 | 0.1 | 287 | 75.6 | 43.9 | 35 | 53.1 |
| 469 | 1489 | 3782 | 3801 | 51.3 | 50 | 3989 | 4445 | 4425 | 50.6 | 42.9 | 0.7 | 664 | 75.5 | 40.5 | 35 | 53.1 |
| 470 | 1490 | 13039 | 13058 | 51.8 | 50 | 3990 | 13155 | 13137 | 52.1 | 52.6 | 0.3 | 117 | 73.4 | 47 | 35 | 52 |
| 471 | 1491 | 3782 | 3801 | 51.3 | 50 | 3991 | 4444 | 4424 | 50.6 | 42.9 | 0.7 | 663 | 75.5 | 40.6 | 35 | 53.1 |
| 472 | 1492 | 13040 | 13059 | 50.9 | 50 | 3992 | 13747 | 13726 | 50.8 | 40.9 | 0.1 | 708 | 76.6 | 43.1 | 35 | 54 |
| 473 | 1493 | 2223 | 2243 | 50.2 | 42.9 | 3993 | 2747 | 2727 | 50 | 42.9 | 0.2 | 525 | 76.9 | 44.6 | 35 | 53.9 |
| 474 | 1494 | 9929 | 9946 | 50 | 50 | 3994 | 10449 | 10431 | 50.9 | 47.4 | 0.9 | 521 | 75.4 | 40.9 | 35 | 52.9 |
| 475 | 1495 | 18077 | 18097 | 51.5 | 47.6 | 3995 | 18702 | 18685 | 50.2 | 50 | 1.4 | 626 | 76.2 | 42.3 | 35 | 53.5 |
| 476 | 1496 | 3360 | 3379 | 50.7 | 45 | 3996 | 3646 | 3625 | 52 | 40.9 | 1.3 | 287 | 75.4 | 43.6 | 35 | 53.1 |
| 477 | 1497 | 26708 | 26731 | 54.2 | 41.7 | 3997 | 27463 | 27443 | 52.7 | 42.9 | 1.5 | 756 | 75.9 | 41.1 | 35 | 54 |
| 478 | 1498 | 26708 | 26731 | 54.2 | 41.7 | 3998 | 27464 | 27444 | 53 | 42.9 | 1.2 | 757 | 75.9 | 41.2 | 35 | 54.1 |
| 479 | 1499 | 26708 | 26731 | 54.2 | 41.7 | 3999 | 27464 | 27445 | 52.4 | 45 | 1.8 | 757 | 75.9 | 41.2 | 35 | 54 |
| 480 | 1500 | 4 | 22 | 52.3 | 52.6 | 4000 | 713 | 695 | 50.7 | 47.4 | 1.6 | 710 | 79 | 49 | 35 | 55.7 |
| 481 | 1501 | 26708 | 26727 | 50 | 45 | 4001 | 27462 | 27443 | 51.4 | 45 | 1.3 | 755 | 75.9 | 41.2 | 35 | 53.2 |
| 482 | 1502 | 3360 | 3380 | 51.4 | 42.9 | 4002 | 3646 | 3625 | 52 | 40.9 | 0.6 | 287 | 75.4 | 43.6 | 35 | 53.3 |
| 483 | 1503 | 26708 | 26727 | 50 | 45 | 4003 | 27463 | 27445 | 50.8 | 42.1 | 0.8 | 756 | 75.9 | 41.1 | 35 | 53.2 |
| 484 | 1504 | 988 | 1006 | 52.2 | 52.6 | 4004 | 1493 | 1474 | 50.8 | 45 | 1.5 | 506 | 76.5 | 43.7 | 35 | 53.9 |
| 485 | 1505 | 12352 | 12375 | 52.9 | 41.7 | 4005 | 12993 | 12975 | 51.4 | 47.4 | 1.5 | 642 | 76.5 | 43 | 35 | 54 |
| 486 | 1506 | 3360 | 3380 | 51.4 | 42.9 | 4006 | 3647 | 3628 | 50.6 | 45 | 0.8 | 288 | 75.5 | 43.8 | 35 | 53.1 |
| 487 | 1507 | 18074 | 18094 | 51.1 | 42.9 | 4007 | 18702 | 18685 | 50.2 | 50 | 0.9 | 629 | 76.2 | 42.3 | 35 | 53.5 |
| 488 | 1508 | 3360 | 3380 | 51.4 | 42.9 | 4008 | 3650 | 3631 | 53.1 | 50 | 1.7 | 291 | 75.6 | 44 | 35 | 53.5 |
| 489 | 1509 | 8374 | 8395 | 52.4 | 45.5 | 4009 | 8928 | 8911 | 51.9 | 50 | 0.6 | 555 | 75.1 | 40 | 35 | 53.2 |
| 490 | 1510 | 9929 | 9946 | 50 | 50 | 4010 | 10449 | 10428 | 51.9 | 40.9 | 1.9 | 521 | 75.4 | 40.9 | 35 | 52.9 |
| 491 | 1511 | 26421 | 26441 | 51.5 | 42.9 | 4011 | 27132 | 27111 | 50.3 | 40.9 | 1.2 | 712 | 77.1 | 44.2 | 35 | 54.2 |
| 492 | 1512 | 10250 | 10274 | 51.6 | 40 | 4012 | 10356 | 10336 | 52.4 | 47.6 | 0.8 | 107 | 70.8 | 42.1 | 35 | 50.2 |
| 493 | 1513 | 18074 | 18093 | 50.3 | 45 | 4013 | 18702 | 18685 | 50.2 | 50 | 0.2 | 629 | 76.2 | 42.3 | 35 | 53.5 |
| 494 | 1514 | 18017 | 18036 | 54.8 | 55 | 4014 | 18220 | 18202 | 54.8 | 52.6 | 0 | 204 | 74.3 | 43.1 | 35 | 53.5 |
| 495 | 1515 | 18017 | 18036 | 54.8 | 55 | 4015 | 18225 | 18206 | 53.7 | 50 | 1.1 | 209 | 74.3 | 43.1 | 35 | 53.2 |
| 496 | 1516 | 18017 | 18036 | 54.8 | 55 | 4016 | 18232 | 18210 | 54.4 | 47.8 | 0.4 | 216 | 74.6 | 43.5 | 35 | 53.6 |
| 497 | 1517 | 18017 | 18036 | 54.8 | 55 | 4017 | 18234 | 18214 | 53.4 | 47.6 | 1.4 | 218 | 74.7 | 43.6 | 35 | 53.4 |
| 498 | 1518 | 18017 | 18036 | 54.8 | 55 | 4018 | 18235 | 18215 | 54.2 | 52.4 | 0.6 | 219 | 74.8 | 43.8 | 35 | 53.7 |
| 499 | 1519 | 18017 | 18036 | 54.8 | 55 | 4019 | 18443 | 18424 | 55.9 | 55 | 1.1 | 427 | 76 | 43.1 | 35 | 54.7 |
| 500 | 1520 | 18012 | 18031 | 53.2 | 55 | 4020 | 18220 | 18202 | 54.8 | 52.6 | 1.7 | 209 | 74.5 | 43.5 | 35 | 53.2 |
| 501 | 1521 | 18013 | 18031 | 50.6 | 52.6 | 4021 | 18223 | 18206 | 51.8 | 50 | 1.1 | 211 | 74.4 | 43.1 | 35 | 52.4 |
| 502 | 1522 | 18013 | 18031 | 50.6 | 52.6 | 4022 | 18231 | 18210 | 52.2 | 45.5 | 1.6 | 219 | 74.6 | 43.4 | 35 | 52.5 |
| 503 | 1523 | 18013 | 18031 | 50.6 | 52.6 | 4023 | 18233 | 18214 | 52 | 50 | 1.4 | 221 | 74.8 | 43.9 | 35 | 52.7 |
| 504 | 1524 | 18013 | 18031 | 50.6 | 52.6 | 4024 | 18233 | 18215 | 51.3 | 52.6 | 0.7 | 221 | 74.8 | 43.9 | 35 | 52.7 |
| 505 | 1525 | 18013 | 18031 | 50.6 | 52.6 | 4025 | 18662 | 18641 | 50.4 | 40.9 | 0.2 | 650 | 76.3 | 42.6 | 35 | 53.7 |
| 506 | 1526 | 18009 | 18029 | 53.3 | 52.4 | 4026 | 18220 | 18202 | 54.8 | 52.6 | 1.6 | 212 | 74.5 | 43.4 | 35 | 53.2 |
| 507 | 1527 | 18011 | 18029 | 51.3 | 52.6 | 4027 | 18223 | 18206 | 51.8 | 50 | 0.5 | 213 | 74.4 | 43.2 | 35 | 52.6 |
| 508 | 1528 | 18011 | 18029 | 51.3 | 52.6 | 4028 | 18231 | 18210 | 52.2 | 45.5 | 0.9 | 221 | 74.7 | 43.4 | 35 | 52.8 |
| 509 | 1529 | 18011 | 18029 | 51.3 | 52.6 | 4029 | 18233 | 18214 | 52 | 50 | 0.7 | 223 | 74.9 | 43.9 | 35 | 52.9 |
| 510 | 1530 | 18011 | 18029 | 51.3 | 52.6 | 4030 | 18233 | 18215 | 51.3 | 52.6 | 0 | 223 | 74.9 | 43.9 | 35 | 52.9 |
| 511 | 1531 | 16374 | 16397 | 52.8 | 41.7 | 4031 | 16774 | 16751 | 53.6 | 41.7 | 0.8 | 401 | 75 | 40.9 | 35 | 53.4 |
| 512 | 1532 | 16378 | 16397 | 50.4 | 45 | 4032 | 16780 | 16760 | 51.4 | 42.9 | 1 | 403 | 75 | 40.9 | 35 | 52.7 |
| 513 | 1533 | 2223 | 2243 | 50.2 | 42.9 | 4033 | 2997 | 2976 | 51.4 | 40.9 | 1.2 | 775 | 76.7 | 43.1 | 35 | 53.9 |
| 514 | 1534 | 2428 | 2447 | 51.5 | 50 | 4034 | 3082 | 3058 | 52.3 | 40 | 0.8 | 655 | 76.3 | 42.6 | 35 | 54 |
| 515 | 1535 | 16548 | 16566 | 54.9 | 52.6 | 4035 | 16774 | 16751 | 53.6 | 41.7 | 1.3 | 227 | 73.9 | 41.4 | 35 | 52.9 |
| 516 | 1536 | 16367 | 16386 | 51.4 | 50 | 4036 | 16774 | 16752 | 52.2 | 43.5 | 0.8 | 408 | 75 | 40.9 | 35 | 53 |
| 517 | 1537 | 3230 | 3249 | 50.1 | 45 | 4037 | 3497 | 3478 | 51.3 | 50 | 1.2 | 268 | 74.4 | 41.4 | 35 | 52.2 |
| 518 | 1538 | 8221 | 8240 | 52.4 | 50 | 4038 | 8920 | 8901 | 53.4 | 50 | 1 | 700 | 75.3 | 40 | 35 | 53.6 |
| 519 | 1539 | 3232 | 3252 | 51.1 | 47.6 | 4039 | 3500 | 3481 | 51.2 | 50 | 0.1 | 269 | 74.5 | 41.6 | 35 | 52.5 |
| 520 | 1540 | 3232 | 3252 | 51.1 | 47.6 | 4040 | 3497 | 3478 | 51.3 | 50 | 0.2 | 266 | 74.5 | 41.7 | 35 | 52.6 |
| 521 | 1541 | 16367 | 16386 | 51.4 | 50 | 4041 | 17111 | 17090 | 51.1 | 40.9 | 0.3 | 745 | 76.3 | 42.1 | 35 | 53.8 |
| 522 | 1542 | 16366 | 16385 | 52.9 | 55 | 4042 | 16774 | 16751 | 53.6 | 41.7 | 0.8 | 409 | 75.1 | 41.1 | 35 | 53.5 |
| 523 | 1543 | 9930 | 9948 | 51.5 | 52.6 | 4043 | 10670 | 10649 | 51.3 | 40.9 | 0.2 | 741 | 75.8 | 40.9 | 35 | 53.5 |
| 524 | 1544 | 12370 | 12388 | 50.1 | 47.4 | 4044 | 12996 | 12977 | 50.2 | 40 | 0.2 | 627 | 76.4 | 42.7 | 35 | 53.6 |
| 525 | 1545 | 25354 | 25372 | 50.9 | 52.6 | 4045 | 25650 | 25631 | 51.3 | 45 | 0.4 | 297 | 74.5 | 41.1 | 35 | 52.5 |
| 526 | 1546 | 25354 | 25372 | 50.9 | 52.6 | 4046 | 25651 | 25634 | 50.4 | 50 | 0.5 | 298 | 74.6 | 41.3 | 35 | 52.4 |
| 527 | 1547 | 25354 | 25372 | 50.9 | 52.6 | 4047 | 25772 | 25753 | 51.9 | 50 | 1 | 419 | 74.8 | 40.3 | 35 | 52.8 |
| 528 | 1548 | 1402 | 1422 | 50.2 | 42.9 | 4048 | 1501 | 1482 | 50.5 | 45 | 0.3 | 100 | 72 | 46 | 35 | 50.6 |
| 529 | 1549 | 25354 | 25372 | 50.9 | 52.6 | 4049 | 25831 | 25809 | 51.4 | 43.5 | 0.5 | 478 | 75 | 40.2 | 35 | 52.8 |
| 530 | 1550 | 3797 | 3815 | 50.9 | 47.4 | 4050 | 4434 | 4416 | 51.5 | 52.6 | 0.5 | 638 | 75.4 | 40.3 | 35 | 53.1 |
| 531 | 1551 | 25354 | 25372 | 50.9 | 52.6 | 4051 | 25831 | 25810 | 50.7 | 45.5 | 0.2 | 478 | 75 | 40.2 | 35 | 52.8 |
| 532 | 1552 | 3797 | 3815 | 50.9 | 47.4 | 4052 | 4435 | 4417 | 50.5 | 52.6 | 0.5 | 639 | 75.4 | 40.4 | 35 | 53 |
| 533 | 1553 | 24481 | 24500 | 50.1 | 45 | 4053 | 24938 | 24921 | 50.4 | 50 | 0.3 | 458 | 75.6 | 41.9 | 35 | 53.1 |
| 534 | 1554 | 25348 | 25366 | 51.2 | 47.4 | 4054 | 25831 | 25809 | 51.4 | 43.5 | 0.2 | 484 | 75 | 40.3 | 35 | 53 |
| 535 | 1555 | 25348 | 25366 | 51.2 | 47.4 | 4055 | 25831 | 25810 | 50.7 | 45.5 | 0.4 | 484 | 75 | 40.3 | 35 | 52.8 |
| 536 | 1556 | 24419 | 24440 | 52.3 | 45.5 | 4056 | 25080 | 25062 | 53.5 | 52.6 | 1.2 | 662 | 75.7 | 41.1 | 35 | 53.8 |
| 537 | 1557 | 24420 | 24440 | 50.8 | 42.9 | 4057 | 24527 | 24508 | 50.5 | 45 | 0.3 | 108 | 70.7 | 41.7 | 35 | 49.8 |
| 538 | 1558 | 25348 | 25365 | 50.4 | 50 | 4058 | 25650 | 25631 | 51.3 | 45 | 0.9 | 303 | 74.6 | 41.3 | 35 | 52.4 |
| 539 | 1559 | 25348 | 25365 | 50.4 | 50 | 4059 | 25651 | 25634 | 50.4 | 50 | 0.1 | 304 | 74.7 | 41.4 | 35 | 52.5 |
| 540 | 1560 | 25348 | 25365 | 50.4 | 50 | 4060 | 25831 | 25809 | 51.4 | 43.5 | 1 | 484 | 75 | 40.3 | 35 | 52.7 |
| 541 | 1561 | 25348 | 25365 | 50.4 | 50 | 4061 | 25831 | 25810 | 50.7 | 45.5 | 0.3 | 484 | 75 | 40.3 | 35 | 52.7 |
| 542 | 1562 | 28618 | 28636 | 52.5 | 52.6 | 4062 | 29298 | 29280 | 51.4 | 52.6 | 1.1 | 681 | 78.3 | 47.3 | 35 | 55.3 |
| 543 | 1563 | 8867 | 8887 | 52.3 | 47.6 | 4063 | 9317 | 9297 | 50.5 | 42.9 | 1.8 | 451 | 75.5 | 41.7 | 35 | 53.1 |
| 544 | 1564 | 28820 | 28838 | 53.7 | 52.6 | 4064 | 29301 | 29282 | 55.3 | 55 | 1.6 | 482 | 77.1 | 45.4 | 35 | 55.2 |
| 545 | 1565 | 27365 | 27385 | 53.2 | 47.6 | 4065 | 27464 | 27443 | 54 | 45.5 | 0.8 | 100 | 70.8 | 43 | 35 | 50.6 |
| 546 | 1566 | 28820 | 28839 | 54.3 | 50 | 4066 | 29306 | 29288 | 53.5 | 52.6 | 0.8 | 487 | 77.1 | 45.4 | 35 | 55.1 |
| 547 | 1567 | 28820 | 28839 | 54.3 | 50 | 4067 | 29301 | 29282 | 55.3 | 55 | 1 | 482 | 77.1 | 45.4 | 35 | 55.4 |
| 548 | 1568 | 28821 | 28840 | 51.8 | 45 | 4068 | 29306 | 29288 | 53.5 | 52.6 | 1.7 | 486 | 77.1 | 45.3 | 35 | 54.6 |
| 549 | 1569 | 27370 | 27389 | 50.1 | 45 | 4069 | 27675 | 27656 | 50 | 40 | 0.1 | 306 | 74.2 | 40.2 | 35 | 52 |
| 550 | 1570 | 28820 | 28840 | 54.8 | 47.6 | 4070 | 29301 | 29282 | 55.3 | 55 | 0.4 | 482 | 77.1 | 45.4 | 35 | 55.5 |
| 551 | 1571 | 27370 | 27389 | 50.1 | 45 | 4071 | 27674 | 27654 | 51.9 | 42.9 | 1.8 | 305 | 74.2 | 40.3 | 35 | 52.1 |
| 552 | 1572 | 2429 | 2447 | 50.2 | 47.4 | 4072 | 3188 | 3167 | 50.2 | 40.9 | 0 | 760 | 76.6 | 42.9 | 35 | 53.8 |
| 553 | 1573 | 27375 | 27392 | 50 | 50 | 4073 | 27675 | 27656 | 50 | 40 | 0 | 301 | 74.1 | 40.2 | 35 | 52 |
| 554 | 1574 | 27375 | 27392 | 50 | 50 | 4074 | 27674 | 27654 | 51.9 | 42.9 | 1.9 | 300 | 74.2 | 40.3 | 35 | 52 |
| 555 | 1575 | 19795 | 19814 | 50.4 | 45 | 4075 | 19916 | 19895 | 50.2 | 40.9 | 0.2 | 122 | 71.8 | 42.6 | 35 | 50.5 |
| 556 | 1576 | 3168 | 3189 | 51 | 45.5 | 4076 | 3646 | 3625 | 52 | 40.9 | 1.1 | 479 | 75.7 | 42 | 35 | 53.4 |
| 557 | 1577 | 3168 | 3189 | 51 | 45.5 | 4077 | 3647 | 3628 | 50.6 | 45 | 0.4 | 480 | 75.8 | 42.1 | 35 | 53.3 |
| 558 | 1578 | 18011 | 18029 | 51.3 | 52.6 | 4078 | 18662 | 18641 | 50.4 | 40.9 | 0.9 | 652 | 76.3 | 42.6 | 35 | 53.7 |
| 559 | 1579 | 985 | 1004 | 51.1 | 50 | 4079 | 1493 | 1474 | 50.8 | 45 | 0.3 | 509 | 76.5 | 43.6 | 35 | 53.9 |
| 560 | 1580 | 12965 | 12985 | 51.2 | 42.9 | 4080 | 13547 | 13528 | 50.2 | 45 | 0.9 | 583 | 77.3 | 45.3 | 35 | 54.3 |
| 561 | 1581 | 2427 | 2445 | 52.1 | 52.6 | 4081 | 3188 | 3167 | 50.2 | 40.9 | 1.9 | 762 | 76.7 | 43 | 35 | 53.8 |
| 562 | 1582 | 3360 | 3381 | 52.1 | 40.9 | 4082 | 3650 | 3631 | 53.1 | 50 | 1 | 291 | 75.6 | 44 | 35 | 53.7 |
| 563 | 1583 | 12726 | 12746 | 51.3 | 47.6 | 4083 | 12911 | 12892 | 50.5 | 50 | 0.8 | 186 | 73.5 | 41.9 | 35 | 51.7 |
| 564 | 1584 | 19800 | 19817 | 50.4 | 50 | 4084 | 19917 | 19896 | 50.9 | 45.5 | 0.5 | 118 | 71.9 | 43.2 | 35 | 50.6 |
| 565 | 1585 | 1402 | 1426 | 54.1 | 40 | 4085 | 1501 | 1478 | 54.6 | 41.7 | 0.5 | 100 | 72 | 46 | 35 | 51.8 |
| 566 | 1586 | 2427 | 2445 | 52.1 | 52.6 | 4086 | 3082 | 3058 | 52.3 | 40 | 0.2 | 656 | 76.4 | 42.7 | 35 | 54.2 |
| 567 | 1587 | 8867 | 8887 | 52.3 | 47.6 | 4087 | 9257 | 9238 | 50.5 | 45 | 1.8 | 391 | 75.1 | 41.2 | 35 | 52.8 |
| 568 | 1588 | 8867 | 8887 | 52.3 | 47.6 | 4088 | 9249 | 9231 | 50.8 | 47.4 | 1.5 | 383 | 75.2 | 41.5 | 35 | 53 |
| 569 | 1589 | 8374 | 8394 | 51 | 42.9 | 4089 | 8928 | 8911 | 51.9 | 50 | 0.8 | 555 | 75.1 | 40 | 35 | 53 |
| 570 | 1590 | 8867 | 8887 | 52.3 | 47.6 | 4090 | 9249 | 9230 | 51.5 | 45 | 0.8 | 383 | 75.2 | 41.5 | 35 | 53.2 |
| 571 | 1591 | 28964 | 28984 | 54.3 | 52.4 | 4091 | 29301 | 29282 | 55.3 | 55 | 1 | 338 | 76.5 | 45.3 | 35 | 54.9 |
| 572 | 1592 | 8867 | 8887 | 52.3 | 47.6 | 4092 | 9249 | 9229 | 53 | 47.6 | 0.6 | 383 | 75.2 | 41.5 | 35 | 53.4 |
| 573 | 1593 | 12962 | 12980 | 50.7 | 47.4 | 4093 | 13547 | 13528 | 50.2 | 45 | 0.5 | 586 | 77.4 | 45.4 | 35 | 54.3 |
| 574 | 1594 | 9931 | 9950 | 50.2 | 45 | 4094 | 10605 | 10588 | 51.1 | 50 | 0.9 | 675 | 75.8 | 41.2 | 35 | 53.2 |
| 575 | 1595 | 19801 | 19819 | 53.2 | 52.6 | 4095 | 19918 | 19896 | 52.2 | 43.5 | 1 | 118 | 71.9 | 43.2 | 35 | 51.1 |
| 576 | 1596 | 9055 | 9079 | 52.8 | 40 | 4096 | 9376 | 9355 | 51 | 40.9 | 1.8 | 322 | 75.1 | 42.2 | 35 | 53 |
| 577 | 1597 | 19878 | 19899 | 50.5 | 40.9 | 4097 | 20033 | 20016 | 50.4 | 50 | 0.1 | 156 | 73.4 | 43.6 | 35 | 51.6 |
| 578 | 1598 | 17608 | 17628 | 50.9 | 42.9 | 4098 | 18233 | 18214 | 52 | 50 | 1.1 | 626 | 75.3 | 40.3 | 35 | 53.1 |
| 579 | 1599 | 17608 | 17627 | 50.2 | 45 | 4099 | 18233 | 18214 | 52 | 50 | 1.8 | 626 | 75.3 | 40.3 | 35 | 52.9 |
| 580 | 1600 | 29179 | 29199 | 51.4 | 42.9 | 4100 | 29358 | 29339 | 52.8 | 50 | 1.4 | 180 | 74.8 | 45.6 | 35 | 52.9 |
| 581 | 1601 | 29182 | 29202 | 51.2 | 42.9 | 4101 | 29358 | 29339 | 52.8 | 50 | 1.6 | 177 | 74.6 | 45.2 | 35 | 52.7 |
| 582 | 1602 | 4 | 22 | 52.3 | 52.6 | 4102 | 253 | 233 | 51.8 | 47.6 | 0.5 | 250 | 76.2 | 46.4 | 35 | 54 |
| 583 | 1603 | 8221 | 8240 | 52.4 | 50 | 4103 | 8920 | 8902 | 52.8 | 52.6 | 0.3 | 700 | 75.3 | 40 | 35 | 53.6 |
| 584 | 1604 | 16554 | 16572 | 53.7 | 52.6 | 4104 | 16774 | 16751 | 53.6 | 41.7 | 0.1 | 221 | 73.7 | 41.2 | 35 | 52.8 |
| 585 | 1605 | 16555 | 16572 | 50.3 | 50 | 4105 | 16711 | 16691 | 51 | 42.9 | 0.7 | 157 | 73.4 | 43.3 | 35 | 51.6 |
| 586 | 1606 | 29186 | 29205 | 50.1 | 40 | 4106 | 29412 | 29393 | 50.3 | 45 | 0.3 | 227 | 75.4 | 44.9 | 35 | 52.9 |
| 587 | 1607 | 2429 | 2447 | 50.2 | 47.4 | 4107 | 3052 | 3033 | 50.3 | 50 | 0.1 | 624 | 76.3 | 42.6 | 35 | 53.6 |
| 588 | 1608 | 29182 | 29205 | 54.6 | 41.7 | 4108 | 29358 | 29339 | 52.8 | 50 | 1.7 | 177 | 74.6 | 45.2 | 35 | 53.2 |
| 589 | 1609 | 4 | 22 | 52.3 | 52.6 | 4109 | 255 | 235 | 51.3 | 47.6 | 1 | 252 | 76.3 | 46.4 | 35 | 53.9 |
| 590 | 1610 | 3230 | 3249 | 50.1 | 45 | 4110 | 3500 | 3481 | 51.2 | 50 | 1.1 | 271 | 74.4 | 41.3 | 35 | 52.2 |
| 591 | 1611 | 13040 | 13059 | 50.9 | 50 | 4111 | 13177 | 13156 | 50.4 | 40.9 | 0.5 | 138 | 73.7 | 45.7 | 35 | 51.8 |
| 592 | 1612 | 16551 | 16568 | 51.1 | 50 | 4112 | 17039 | 17022 | 51.4 | 50 | 0.3 | 489 | 75.8 | 42.1 | 35 | 53.5 |
| 593 | 1613 | 19995 | 20012 | 50.4 | 50 | 4113 | 20615 | 20597 | 50.6 | 47.4 | 0.2 | 621 | 75.3 | 40.1 | 35 | 52.9 |
| 594 | 1614 | 19995 | 20013 | 51.8 | 52.6 | 4114 | 20615 | 20597 | 50.6 | 47.4 | 1.2 | 621 | 75.3 | 40.1 | 35 | 53 |
| 595 | 1615 | 12370 | 12388 | 50.1 | 47.4 | 4115 | 12993 | 12975 | 51.4 | 47.4 | 1.3 | 624 | 76.4 | 42.9 | 35 | 53.6 |
| 596 | 1616 | 8374 | 8393 | 51.2 | 45 | 4116 | 8928 | 8911 | 51.9 | 50 | 0.7 | 555 | 75.1 | 40 | 35 | 53 |
| 597 | 1617 | 24174 | 24194 | 50.9 | 42.9 | 4117 | 24936 | 24919 | 51.8 | 50 | 0.8 | 763 | 75.8 | 41 | 35 | 53.5 |
| 598 | 1618 | 24179 | 24198 | 51 | 45 | 4118 | 24936 | 24919 | 51.8 | 50 | 0.7 | 758 | 75.8 | 41 | 35 | 53.5 |
| 599 | 1619 | 7679 | 7698 | 50.6 | 50 | 4119 | 8049 | 8032 | 50.4 | 50 | 0.2 | 371 | 75.4 | 42.3 | 35 | 53 |
| 600 | 1620 | 13177 | 13197 | 50.3 | 42.9 | 4120 | 13320 | 13300 | 51.4 | 47.6 | 1.1 | 144 | 73.2 | 43.8 | 35 | 51.4 |
| 601 | 1621 | 24179 | 24200 | 53.3 | 40.9 | 4121 | 24934 | 24913 | 53.4 | 45.5 | 0.2 | 756 | 75.8 | 41 | 35 | 54.2 |
| 602 | 1622 | 9927 | 9945 | 50.8 | 52.6 | 4122 | 10670 | 10649 | 51.3 | 40.9 | 0.5 | 744 | 75.7 | 40.9 | 35 | 53.4 |
| 603 | 1623 | 2427 | 2445 | 52.1 | 52.6 | 4123 | 3052 | 3033 | 50.3 | 50 | 1.8 | 626 | 76.4 | 42.8 | 35 | 53.6 |
| 604 | 1624 | 24418 | 24436 | 50 | 47.4 | 4124 | 24527 | 24507 | 51 | 42.9 | 1 | 110 | 71.3 | 42.7 | 35 | 50 |
| 605 | 1625 | 24417 | 24436 | 52.6 | 50 | 4125 | 24517 | 24494 | 53.2 | 41.7 | 0.6 | 101 | 71.1 | 43.6 | 35 | 50.6 |
| 606 | 1626 | 8375 | 8396 | 51.8 | 45.5 | 4126 | 8929 | 8911 | 53.4 | 52.6 | 1.6 | 555 | 75.1 | 40 | 35 | 53.2 |
| 607 | 1627 | 24418 | 24439 | 52.9 | 45.5 | 4127 | 25080 | 25062 | 53.5 | 52.6 | 0.6 | 663 | 75.8 | 41.2 | 35 | 54 |
| 608 | 1628 | 18074 | 18094 | 51.1 | 42.9 | 4128 | 18662 | 18641 | 50.4 | 40.9 | 0.6 | 589 | 76.2 | 42.6 | 36 | 53.6 |
| 609 | 1629 | 18074 | 18094 | 51.1 | 42.9 | 4129 | 18632 | 18611 | 50.2 | 40.9 | 0.9 | 559 | 76.2 | 42.8 | 36 | 53.5 |
| 610 | 1630 | 13231 | 13251 | 50.1 | 42.9 | 4130 | 13545 | 13527 | 50.3 | 52.6 | 0.2 | 315 | 77 | 47 | 36 | 54 |
| 611 | 1631 | 7400 | 7417 | 50.2 | 50 | 4131 | 8188 | 8169 | 50.5 | 45 | 0.3 | 789 | 76.4 | 42.2 | 36 | 53.6 |
| 612 | 1632 | 3792 | 3811 | 54 | 55 | 4132 | 4446 | 4424 | 52.4 | 43.5 | 1.6 | 655 | 75.5 | 40.6 | 36 | 53.7 |
| 613 | 1633 | 25782 | 25805 | 52.1 | 41.7 | 4133 | 26182 | 26161 | 51.2 | 40.9 | 0.9 | 401 | 74.7 | 40.1 | 36 | 52.7 |
| 614 | 1634 | 13230 | 13251 | 52.4 | 45.5 | 4134 | 13545 | 13526 | 52.9 | 55 | 0.5 | 316 | 77.1 | 47.2 | 36 | 54.8 |
| 615 | 1635 | 985 | 1004 | 51.1 | 50 | 4135 | 1480 | 1462 | 51.6 | 47.4 | 0.5 | 496 | 76.4 | 43.5 | 36 | 53.9 |
| 616 | 1636 | 7400 | 7417 | 50.2 | 50 | 4136 | 8049 | 8032 | 50.4 | 50 | 0.2 | 650 | 76.1 | 42.2 | 36 | 53.5 |
| 617 | 1637 | 13176 | 13197 | 52.7 | 45.5 | 4137 | 13545 | 13526 | 52.9 | 55 | 0.2 | 370 | 77 | 46.2 | 36 | 54.8 |
| 618 | 1638 | 25782 | 25806 | 53.5 | 40 | 4138 | 26183 | 26162 | 52.8 | 45.5 | 0.7 | 402 | 74.7 | 40.3 | 36 | 53.3 |
| 619 | 1639 | 13176 | 13196 | 51.4 | 47.6 | 4139 | 13547 | 13528 | 50.2 | 45 | 1.2 | 372 | 76.9 | 46 | 36 | 54 |
| 620 | 1640 | 12938 | 12956 | 50.1 | 47.4 | 4140 | 13155 | 13138 | 50.4 | 50 | 0.3 | 218 | 75.4 | 45.4 | 36 | 52.9 |
| 621 | 1641 | 18080 | 18099 | 53 | 50 | 4141 | 18712 | 18693 | 54.8 | 55 | 1.9 | 633 | 76.3 | 42.7 | 36 | 54.4 |
| 622 | 1642 | 9140 | 9159 | 50.1 | 45 | 4142 | 9375 | 9354 | 50.4 | 40.9 | 0.3 | 236 | 74.6 | 42.8 | 36 | 52.3 |
| 623 | 1643 | 7725 | 7742 | 50 | 50 | 4143 | 8054 | 8035 | 50.4 | 50 | 0.4 | 330 | 75 | 41.8 | 36 | 52.6 |
| 624 | 1644 | 9922 | 9941 | 51.3 | 50 | 4144 | 10455 | 10435 | 50.5 | 42.9 | 0.8 | 534 | 75.3 | 40.6 | 36 | 52.9 |
| 625 | 1645 | 12938 | 12957 | 50.9 | 45 | 4145 | 13155 | 13138 | 50.4 | 50 | 0.5 | 218 | 75.4 | 45.4 | 36 | 53 |
| 626 | 1646 | 12366 | 12384 | 51.7 | 52.6 | 4146 | 12996 | 12977 | 50.2 | 40 | 1.4 | 631 | 76.4 | 42.6. | 36 | 53.6 |
| 627 | 1647 | 7617 | 7636 | 50.9 | 50 | 4147 | 8049 | 8032 | 50.4 | 50 | 0.6 | 433 | 75.7 | 42.3 | 36 | 53.2 |
| 628 | 1648 | 2671 | 2692 | 52.1 | 40.9 | 4148 | 3188 | 3167 | 50.2 | 40.9 | 2 | 518 | 75.6 | 41.5 | 36 | 53.1 |
| 629 | 1649 | 26039 | 26057 | 52.6 | 52.6 | 4149 | 26183 | 26164 | 51 | 45 | 1.6 | 145 | 71.9 | 40.7 | 36 | 50.8 |
| 630 | 1650 | 11540 | 11557 | 50.4 | 50 | 4150 | 11727 | 11708 | 50.4 | 45 | 0.1 | 188 | 73.1 | 41 | 36 | 51.4 |
| 631 | 1651 | 12962 | 12980 | 50.7 | 47.4 | 4151 | 13545 | 13527 | 50.3 | 52.6 | 0.4 | 584 | 77.4 | 45.5 | 36 | 54.4 |
| 632 | 1652 | 12961 | 12980 | 53.2 | 50 | 4152 | 13545 | 13526 | 52.9 | 55 | 0.4 | 585 | 77.5 | 45.6 | 36 | 55.2 |
| 633 | 1653 | 9055 | 9079 | 52.8 | 40 | 4153 | 9369 | 9350 | 51.5 | 50 | 1.4 | 315 | 75.3 | 42.9 | 36 | 53.3 |
| 634 | 1654 | 12965 | 12985 | 51.2 | 42.9 | 4154 | 13545 | 13527 | 50.3 | 52.6 | 0.9 | 581 | 77.4 | 45.4 | 36 | 54.3 |
| 635 | 1655 | 26039 | 26058 | 54 | 55 | 4155 | 26693 | 26674 | 54.8 | 55 | 0.8 | 655 | 75.7 | 41.1 | 36 | 54.3 |
| 636 | 1656 | 26039 | 26058 | 54 | 55 | 4156 | 26692 | 26673 | 52.6 | 50 | 1.4 | 654 | 75.7 | 41 | 36 | 53.8 |
| 637 | 1657 | 26039 | 26058 | 54 | 55 | 4157 | 26688 | 26669 | 52.1 | 45 | 2 | 650 | 75.6 | 40.8 | 36 | 53.6 |
| 638 | 1658 | 26039 | 26058 | 54 | 55 | 4158 | 26684 | 26666 | 53.4 | 52.6 | 0.6 | 646 | 75.6 | 40.9 | 36 | 54.1 |
| 639 | 1659 | 26039 | 26058 | 54 | 55 | 4159 | 26683 | 26665 | 52.7 | 52.6 | 1.4 | 645 | 75.6 | 40.9 | 36 | 53.8 |
| 640 | 1660 | 12965 | 12985 | 51.2 | 42.9 | 4160 | 13545 | 13526 | 52.9 | 55 | 1.7 | 581 | 77.4 | 45.4 | 36 | 54.6 |
| 641 | 1661 | 26039 | 26058 | 54 | 55 | 4161 | 26183 | 26162 | 52.8 | 45.5 | 1.2 | 145 | 71.9 | 40.7 | 36 | 51.3 |
| 642 | 1662 | 9055 | 9079 | 52.8 | 40 | 4162 | 9365 | 9347 | 53 | 52.6 | 0.2 | 311 | 75.3 | 42.8 | 36 | 53.6 |
| 643 | 1663 | 19795 | 19814 | 50.4 | 45 | 4163 | 19922 | 19902 | 50 | 42.9 | 0.4 | 128 | 72.2 | 43 | 36 | 50.7 |
| 644 | 1664 | 12965 | 12988 | 54 | 41.7 | 4164 | 13545 | 13526 | 52.9 | 55 | 1.2 | 581 | 77.4 | 45.4 | 36 | 55.1 |
| 645 | 1665 | 26040 | 26061 | 56.4 | 54.5 | 4165 | 26693 | 26674 | 54.8 | 55 | 1.6 | 654 | 75.7 | 41.1 | 36 | 54.6 |
| 646 | 1666 | 26040 | 26061 | 56.4 | 54.5 | 4166 | 26693 | 26673 | 55.3 | 52.4 | 1.1 | 654 | 75.7 | 41.1 | 36 | 54.7 |
| 647 | 1667 | 26040 | 26061 | 56.4 | 54.5 | 4167 | 26690 | 26669 | 56.3 | 50 | 0.1 | 651 | 75.7 | 41 | 36 | 55 |
| 648 | 1668 | 26040 | 26061 | 56.4 | 54.5 | 4168 | 26685 | 26666 | 54.8 | 55 | 1.6 | 646 | 75.7 | 41 | 36 | 54.5 |
| 649 | 1669 | 26040 | 26061 | 56.4 | 54.5 | 4169 | 26685 | 26665 | 55.3 | 52.4 | 1.1 | 646 | 75.7 | 41 | 36 | 54.7 |
| 650 | 1670 | 18011 | 18031 | 54.5 | 52.4 | 4170 | 18443 | 18424 | 55.9 | 55 | 1.4 | 433 | 76.1 | 43.2 | 36 | 54.7 |
| 651 | 1671 | 7876 | 7895 | 51.5 | 45 | 4171 | 8049 | 8032 | 50.4 | 50 | 1.2 | 174 | 73.2 | 42 | 36 | 51.5 |
| 652 | 1672 | 3230 | 3249 | 50.1 | 45 | 4172 | 3646 | 3625 | 52 | 40.9 | 1.9 | 417 | 75.2 | 41.2 | 36 | 52.8 |
| 653 | 1673 | 19795 | 19814 | 50.4 | 45 | 4173 | 19920 | 19899 | 50.2 | 40.9 | 0.3 | 126 | 72.1 | 42.9 | 36 | 50.6 |
| 654 | 1674 | 12366 | 12384 | 51.7 | 52.6 | 4174 | 12993 | 12975 | 51.4 | 47.4 | 0.3 | 628 | 76.5 | 43 | 36 | 54 |
| 655 | 1675 | 19793 | 19814 | 54 | 50 | 4175 | 20544 | 20524 | 52.3 | 47.6 | 1.7 | 752 | 75.4 | 40 | 36 | 53.6 |
| 656 | 1676 | 12366 | 12384 | 51.7 | 52.6 | 4176 | 12911 | 12892 | 50.5 | 50 | 1.2 | 546 | 76.1 | 42.5 | 36 | 53.5 |
| 657 | 1677 | 7728 | 7746 | 51.7 | 52.6 | 4177 | 8188 | 8168 | 50.4 | 42.9 | 1.3 | 461 | 75.6 | 41.9 | 36 | 53.1 |
| 658 | 1678 | 26421 | 26441 | 51.5 | 42.9 | 4178 | 27084 | 27063 | 51.6 | 40.9 | 0.2 | 664 | 77.3 | 45 | 36 | 54.7 |
| 659 | 1679 | 9929 | 9946 | 50 | 50 | 4179 | 10455 | 10434 | 51.1 | 40.9 | 1.1 | 527 | 75.3 | 40.6 | 36 | 52.8 |
| 660 | 1680 | 26421 | 26441 | 51.5 | 42.9 | 4180 | 27083 | 27062 | 50.7 | 40.9 | 0.8 | 663 | 77.4 | 45.1 | 36 | 54.5 |
| 661 | 1681 | 12236 | 12256 | 51.2 | 42.9 | 4181 | 12999 | 12980 | 50.6 | 40 | 0.6 | 764 | 76.4 | 42.4 | 36 | 53.8 |
| 662 | 1682 | 26421 | 26441 | 51.5 | 42.9 | 4182 | 26694 | 26677 | 51.4 | 50 | 0 | 274 | 74.9 | 42.7 | 36 | 53 |
| 663 | 1683 | 9929 | 9946 | 50 | 50 | 4183 | 10183 | 10166 | 50.9 | 50 | 0.8 | 255 | 75.3 | 43.9 | 36 | 52.8 |
| 664 | 1684 | 12234 | 12252 | 50.6 | 47.4 | 4184 | 13000 | 12981 | 51.1 | 45 | 0.5 | 767 | 76.4 | 42.5 | 36 | 53.8 |
| 665 | 1685 | 8868 | 8889 | 50.4 | 40.9 | 4185 | 9254 | 9236 | 50.6 | 47.4 | 0.2 | 387 | 75 | 41.1 | 36 | 52.7 |
| 666 | 1686 | 9130 | 9150 | 51.3 | 42.9 | 4186 | 9597 | 9577 | 50.3 | 42.9 | 1 | 468 | 75.4 | 41.2 | 36 | 52.9 |
| 667 | 1687 | 9935 | 9955 | 50.4 | 42.9 | 4187 | 10605 | 10588 | 51.1 | 50 | 0.7 | 671 | 75.8 | 41.1 | 36 | 53.2 |
| 668 | 1688 | 26421 | 26441 | 51.5 | 42.9 | 4188 | 26587 | 26569 | 52 | 47.4 | 0.5 | 167 | 72.3 | 40.1 | 36 | 51.2 |
| 669 | 1689 | 9130 | 9150 | 51.3 | 42.9 | 4189 | 9597 | 9576 | 51 | 40.9 | 0.3 | 468 | 75.4 | 41.2 | 36 | 53.2 |
| 670 | 1690 | 26708 | 26727 | 50 | 45 | 4190 | 27466 | 27449 | 51 | 50 | 0.9 | 759 | 76 | 41.4 | 36 | 53.3 |
| 671 | 1691 | 9130 | 9150 | 51.3 | 42.9 | 4191 | 9375 | 9354 | 50.4 | 40.9 | 0.9 | 246 | 74.7 | 42.7 | 36 | 52.5 |
| 672 | 1692 | 10246 | 10266 | 50.4 | 47.6 | 4192 | 10608 | 10589 | 51 | 50 | 0.5 | 363 | 74.5 | 40.2 | 36 | 52.4 |
| 673 | 1693 | 9924 | 9944 | 53.1 | 52.4 | 4193 | 10449 | 10425 | 54.6 | 40 | 1.5 | 526 | 75.4 | 40.9 | 36 | 53.8 |
| 674 | 1694 | 12366 | 12384 | 51.7 | 52.6 | 4194 | 12911 | 12891 | 51.2 | 47.6 | 0.5 | 546 | 76.1 | 42.5 | 36 | 53.7 |
| 675 | 1695 | 26708 | 26731 | 54.2 | 41.7 | 4195 | 27466 | 27448 | 52.3 | 52.6 | 1.9 | 759 | 76 | 41.4 | 36 | 54 |
| 676 | 1696 | 8867 | 8888 | 52.7 | 45.5 | 4196 | 9107 | 9086 | 51.6 | 45.5 | 1.1 | 241 | 74.1 | 41.5 | 36 | 52.5 |
| 677 | 1697 | 9131 | 9151 | 50.4 | 42.9 | 4197 | 9597 | 9577 | 50.3 | 42.9 | 0.1 | 467 | 75.4 | 41.3 | 36 | 53 |
| 678 | 1698 | 9131 | 9151 | 50.4 | 42.9 | 4198 | 9597 | 9576 | 51 | 40.9 | 0.6 | 467 | 75.4 | 41.3 | 36 | 53 |
| 679 | 1699 | 10242 | 10265 | 51.2 | 41.7 | 4199 | 10608 | 10589 | 51 | 50 | 0.3 | 367 | 74.5 | 40.1 | 36 | 52.5 |
| 680 | 1700 | 27361 | 27380 | 52.4 | 55 | 4200 | 27468 | 27451 | 51.1 | 50 | 1.3 | 108 | 72.3 | 45.4 | 36 | 51 |
| 681 | 1701 | 27361 | 27380 | 52.4 | 55 | 4201 | 27467 | 27450 | 52.1 | 50 | 0.3 | 107 | 72.4 | 45.8 | 36 | 51.4 |
| 682 | 1702 | 27361 | 27380 | 52.4 | 55 | 4202 | 27466 | 27449 | 51 | 50 | 1.4 | 106 | 72.5 | 46.2 | 36 | 51.1 |
| 683 | 1703 | 9926 | 9944 | 50.5 | 52.6 | 4203 | 10449 | 10428 | 51.9 | 40.9 | 1.4 | 524 | 75.4 | 40.8 | 36 | 53 |
| 684 | 1704 | 9926 | 9944 | 50.5 | 52.6 | 4204 | 10449 | 10431 | 50.9 | 47.4 | 0.5 | 524 | 75.4 | 40.8 | 36 | 53 |
| 685 | 1705 | 19802 | 19820 | 53 | 52.6 | 4205 | 19922 | 19901 | 51.5 | 45.5 | 1.4 | 121 | 72.3 | 43.8 | 36 | 51.2 |
| 686 | 1706 | 27361 | 27380 | 52.4 | 55 | 4206 | 27462 | 27443 | 51.4 | 45 | 1 | 102 | 71.8 | 45.1 | 36 | 50.8 |
| 687 | 1707 | 10140 | 10159 | 52.4 | 50 | 4207 | 10605 | 10588 | 51.1 | 50 | 1.3 | 466 | 75 | 40.3 | 36 | 52.9 |
| 688 | 1708 | 16366 | 16384 | 50.3 | 52.6 | 4208 | 16777 | 16758 | 51.5 | 50 | 1.2 | 412 | 75.1 | 41 | 36 | 52.8 |
| 689 | 1709 | 16366 | 16385 | 52.9 | 55 | 4209 | 16781 | 16761 | 51.3 | 47.6 | 1.6 | 416 | 75.1 | 41.1 | 36 | 53.1 |
| 690 | 1710 | 985 | 1008 | 56.1 | 50 | 4210 | 1484 | 1464 | 54.3 | 47.6 | 1.8 | 500 | 76.4 | 43.6 | 36 | 54.9 |
| 691 | 1711 | 16366 | 16385 | 52.9 | 55 | 4211 | 16777 | 16758 | 51.5 | 50 | 1.4 | 412 | 75.1 | 41 | 36 | 53.1 |
| 692 | 1712 | 27366 | 27384 | 52.2 | 52.6 | 4212 | 27466 | 27448 | 52.3 | 52.6 | 0.1 | 101 | 71.5 | 44.6 | 36 | 50.8 |
| 693 | 1713 | 985 | 1008 | 56.1 | 50 | 4213 | 1483 | 1462 | 54.3 | 45.5 | 1.8 | 499 | 76.4 | 43.5 | 36 | 54.8 |
| 694 | 1714 | 2823 | 2844 | 50.4 | 45.5 | 4214 | 3052 | 3033 | 50.3 | 50 | 0.2 | 230 | 74.1 | 41.7 | 36 | 52 |
| 695 | 1715 | 3224 | 3242 | 50.5 | 52.6 | 4215 | 3504 | 3485 | 50.4 | 45 | 0.1 | 281 | 74.7 | 42 | 36 | 52.5 |
| 696 | 1716 | 8867 | 8886 | 50.7 | 50 | 4216 | 9310 | 9291 | 51.2 | 45 | 0.5 | 444 | 75.4 | 41.4 | 36 | 53.1 |
| 697 | 1717 | 8867 | 8886 | 50.7 | 50 | 4217 | 9254 | 9236 | 50.6 | 47.4 | 0.1 | 388 | 75.1 | 41.2 | 36 | 52.8 |
| 698 | 1718 | 9349 | 9367 | 51.7 | 52.6 | 4218 | 9989 | 9968 | 51 | 40.9 | 0.7 | 641 | 75.4 | 40.4 | 36 | 53.2 |
| 699 | 1719 | 8867 | 8887 | 52.3 | 47.6 | 4219 | 9369 | 9350 | 51.5 | 50 | 0.8 | 503 | 75.8 | 42.1 | 36 | 53.6 |
| 700 | 1720 | 8867 | 8887 | 52.3 | 47.6 | 4220 | 9341 | 9322 | 51.1 | 50 | 1.2 | 475 | 75.7 | 41.9 | 36 | 53.4 |
| 701 | 1721 | 9926 | 9944 | 50.5 | 52.6 | 4221 | 10608 | 10589 | 51 | 50 | 0.5 | 683 | 75.8 | 41.1 | 36 | 53.3 |
| 702 | 1722 | 7725 | 7742 | 50 | 50 | 4222 | 8190 | 8172 | 50.3 | 47.4 | 0.3 | 466 | 75.6 | 41.8 | 36 | 53 |
| 703 | 1723 | 9131 | 9151 | 50.4 | 42.9 | 4223 | 9375 | 9354 | 50.4 | 40.9 | 0 | 245 | 74.7 | 42.9 | 36 | 52.5 |
| 704 | 1724 | 3055 | 3075 | 51.8 | 47.6 | 4224 | 3494 | 3473 | 50.4 | 40.9 | 1.4 | 440 | 76 | 43 | 36 | 53.4 |
| 705 | 1725 | 7725 | 7742 | 50 | 50 | 4225 | 8189 | 8170 | 50.6 | 50 | 0.6 | 465 | 75.6 | 41.9 | 36 | 53.1 |
| 706 | 1726 | 2823 | 2844 | 50.4 | 45.5 | 4226 | 3056 | 3038 | 50.8 | 52.6 | 0.3 | 234 | 74.2 | 41.9 | 36 | 52.2 |
| 707 | 1727 | 12370 | 12388 | 50.1 | 47.4 | 4227 | 13155 | 13138 | 50.4 | 50 | 0.3 | 786 | 76.8 | 43.4 | 36 | 53.9 |
| 708 | 1728 | 3055 | 3075 | 51.8 | 47.6 | 4228 | 3209 | 3189 | 50.5 | 47.6 | 1.3 | 155 | 74.1 | 45.2 | 36 | 52.1 |
| 709 | 1729 | 8867 | 8887 | 52.3 | 47.6 | 4229 | 9340 | 9319 | 50.8 | 45.5 | 1.6 | 474 | 75.6 | 41.8 | 36 | 53.3 |
| 710 | 1730 | 27367 | 27385 | 51.4 | 52.6 | 4230 | 27466 | 27448 | 52.3 | 52.6 | 0.9 | 100 | 71.6 | 45 | 36 | 50.6 |
| 711 | 1731 | 14951 | 14975 | 52.2 | 40 | 4231 | 15146 | 15129 | 50.3 | 50 | 1.9 | 196 | 73.2 | 40.8 | 36 | 51.4 |
| 712 | 1732 | 8867 | 8887 | 52.3 | 47.6 | 4232 | 9311 | 9292 | 50.7 | 50 | 1.6 | 445 | 75.4 | 41.6 | 36 | 53.1 |
| 713 | 1733 | 12234 | 12252 | 50.6 | 47.4 | 4233 | 12999 | 12980 | 50.6 | 40 | 0 | 766 | 76.4 | 42.4 | 36 | 53.8 |
| 714 | 1734 | 3055 | 3076 | 52.4 | 45.5 | 4234 | 3495 | 3473 | 51.8 | 43.5 | 0.6 | 441 | 76 | 43.1 | 36 | 53.9 |
| 715 | 1735 | 8867 | 8887 | 52.3 | 47.6 | 4235 | 9109 | 9087 | 50.5 | 43.5 | 1.8 | 243 | 74 | 41.2 | 36 | 52.1 |
| 716 | 1736 | 3055 | 3076 | 52.4 | 45.5 | 4236 | 3209 | 3189 | 50.5 | 47.6 | 2 | 155 | 74.1 | 45.2 | 36 | 52.1 |
| 717 | 1737 | 2671 | 2692 | 52.1 | 40.9 | 4237 | 3053 | 3034 | 50.3 | 50 | 1.8 | 383 | 74.7 | 40.5 | 36 | 52.5 |
| 718 | 1738 | 16981 | 17000 | 51.3 | 50 | 4238 | 17501 | 17481 | 51.2 | 42.9 | 0.1 | 521 | 75.9 | 42.2 | 36 | 53.6 |
| 719 | 1739 | 3796 | 3814 | 50.8 | 52.6 | 4239 | 4444 | 4424 | 50.6 | 42.9 | 0.2 | 649 | 75.5 | 40.5 | 36 | 53.1 |
| 720 | 1740 | 3796 | 3814 | 50.8 | 52.6 | 4240 | 4445 | 4425 | 50.6 | 42.9 | 0.2 | 650 | 75.5 | 40.5 | 36 | 53.1 |
| 721 | 1741 | 27382 | 27401 | 50.8 | 45 | 4241 | 27546 | 27527 | 51.3 | 50 | 0.6 | 165 | 73.7 | 43.6 | 36 | 51.9 |
| 722 | 1742 | 27382 | 27401 | 50.8 | 45 | 4242 | 27541 | 27522 | 50.1 | 45 | 0.6 | 160 | 73.4 | 43.1 | 36 | 51.5 |
| 723 | 1743 | 27383 | 27403 | 50.3 | 42.9 | 4243 | 27546 | 27527 | 51.3 | 50 | 1.1 | 164 | 73.5 | 43.3 | 36 | 51.7 |
| 724 | 1744 | 27383 | 27403 | 50.3 | 42.9 | 4244 | 27541 | 27522 | 50.1 | 45 | 0.1 | 159 | 73.2 | 42.8 | 36 | 51.4 |
| 725 | 1745 | 17789 | 17811 | 52.9 | 43.5 | 4245 | 18220 | 18202 | 54.8 | 52.6 | 1.9 | 432 | 74.9 | 40.5 | 36 | 53.4 |
| 726 | 1746 | 17791 | 17813 | 52.9 | 43.5 | 4246 | 18220 | 18202 | 54.8 | 52.6 | 1.9 | 430 | 74.9 | 40.5 | 36 | 53.4 |
| 727 | 1747 | 18004 | 18023 | 51.1 | 50 | 4247 | 18233 | 18215 | 51.3 | 52.6 | 0.2 | 230 | 75 | 43.9 | 36 | 52.9 |
| 728 | 1748 | 18004 | 18023 | 51.1 | 50 | 4248 | 18231 | 18210 | 52.2 | 45.5 | 1.1 | 228 | 74.7 | 43.4 | 36 | 52.8 |
| 729 | 1749 | 27437 | 27456 | 50.2 | 40 | 4249 | 27546 | 27527 | 51.3 | 50 | 1.1 | 110 | 72.4 | 45.5 | 36 | 50.8 |
| 730 | 1750 | 27437 | 27456 | 50.2 | 40 | 4250 | 27541 | 27522 | 50.1 | 45 | 0.1 | 105 | 71.8 | 44.8 | 36 | 50.4 |
| 731 | 1751 | 18004 | 18023 | 51.1 | 50 | 4251 | 18223 | 18206 | 51.8 | 50 | 0.6 | 220 | 74.5 | 43.2 | 36 | 52.6 |
| 732 | 1752 | 12233 | 12251 | 51.1 | 52.6 | 4252 | 12994 | 12976 | 50.3 | 47.4 | 0.8 | 762 | 76.5 | 42.5 | 36 | 53.7 |
| 733 | 1753 | 7869 | 7889 | 52.5 | 47.6 | 4253 | 8192 | 8172 | 50.9 | 42.9 | 1.6 | 324 | 75.2 | 42.3 | 36 | 53 |
| 734 | 1754 | 3224 | 3242 | 50.5 | 52.6 | 4254 | 3503 | 3484 | 51.5 | 50 | 0.9 | 280 | 74.8 | 42.1 | 36 | 52.6 |
| 735 | 1755 | 3224 | 3242 | 50.5 | 52.6 | 4255 | 3500 | 3481 | 51.2 | 50 | 0.6 | 277 | 74.6 | 41.9 | 36 | 52.5 |
| 736 | 1756 | 3224 | 3242 | 50.5 | 52.6 | 4256 | 3497 | 3478 | 51.3 | 50 | 0.8 | 274 | 74.6 | 42 | 36 | 52.5 |
| 737 | 1757 | 1 | 22 | 54.8 | 50 | 4257 | 204 | 185 | 56.6 | 55 | 1.8 | 204 | 75.1 | 45.1 | 36 | 54.1 |
| 738 | 1758 | 9140 | 9159 | 50.1 | 45 | 4258 | 9597 | 9576 | 51 | 40.9 | 0.9 | 458 | 75.3 | 41.3 | 36 | 52.9 |
| 739 | 1759 | 28179 | 28200 | 50.8 | 40.9 | 4259 | 28671 | 28653 | 50.2 | 52.6 | 0.6 | 493 | 79.7 | 51.7 | 36 | 56 |
| 740 | 1760 | 9140 | 9159 | 50.1 | 45 | 4260 | 9560 | 9540 | 51.6 | 42.9 | 1.5 | 421 | 75.2 | 41.3 | 36 | 52.8 |
| 741 | 1761 | 7728 | 7746 | 51.7 | 52.6 | 4261 | 8189 | 8170 | 50.6 | 50 | 1.1 | 462 | 75.7 | 42 | 36 | 53.3 |
| 742 | 1762 | 9140 | 9159 | 50.1 | 45 | 4262 | 9559 | 9539 | 50.6 | 42.9 | 0.5 | 420 | 75.3 | 41.4 | 36 | 52.8 |
| 743 | 1763 | 12235 | 12253 | 50.1 | 52.6 | 4263 | 12998 | 12979 | 50.1 | 45 | 0 | 764 | 76.5 | 42.5 | 36 | 53.7 |
| 744 | 1764 | 3225 | 3244 | 52.4 | 55 | 4264 | 3503 | 3484 | 51.5 | 50 | 1 | 279 | 74.8 | 42.3 | 36 | 52.9 |
| 745 | 1765 | 14951 | 14975 | 52.2 | 40 | 4265 | 15595 | 15576 | 50.8 | 45 | 1.3 | 645 | 75.5 | 40.6 | 36 | 53.2 |
| 746 | 1766 | 3225 | 3244 | 52.4 | 55 | 4266 | 3500 | 3481 | 51.2 | 50 | 1.3 | 276 | 74.7 | 42 | 36 | 52.7 |
| 747 | 1767 | 12233 | 12251 | 51.1 | 52.6 | 4267 | 12999 | 12980 | 50.6 | 40 | 0.6 | 767 | 76.4 | 42.5 | 36 | 53.8 |
| 748 | 1768 | 3225 | 3244 | 52.4 | 55 | 4268 | 3497 | 3478 | 51.3 | 50 | 1.1 | 273 | 74.7 | 42.1 | 36 | 52.8 |
| 749 | 1769 | 12233 | 12251 | 51.1 | 52.6 | 4269 | 13000 | 12981 | 51.1 | 45 | 0.1 | 768 | 76.5 | 42.6 | 36 | 54 |
| 750 | 1770 | 28395 | 28414 | 51.5 | 45 | 4270 | 28671 | 28653 | 50.2 | 52.6 | 1.3 | 277 | 78.5 | 51.3 | 36 | 55.1 |
| 751 | 1771 | 28395 | 28414 | 51.5 | 45 | 4271 | 28671 | 28652 | 52.8 | 55 | 1.3 | 277 | 78.5 | 51.3 | 36 | 55.5 |
| 752 | 1772 | 9931 | 9950 | 50.2 | 45 | 4272 | 10449 | 10431 | 50.9 | 47.4 | 0.8 | 519 | 75.3 | 40.8 | 36 | 52.9 |
| 753 | 1773 | 12235 | 12253 | 50.1 | 52.6 | 4273 | 12994 | 12976 | 50.3 | 47.4 | 0.2 | 760 | 76.4 | 42.5 | 36 | 53.6 |
| 754 | 1774 | 3359 | 3379 | 51.2 | 42.9 | 4274 | 3650 | 3631 | 53.1 | 50 | 1.9 | 292 | 75.6 | 43.8 | 36 | 53.4 |
| 755 | 1775 | 11543 | 11562 | 50.4 | 40 | 4275 | 12258 | 12238 | 50.3 | 42.9 | 0.2 | 716 | 76.1 | 41.9 | 36 | 53.5 |
| 756 | 1776 | 28396 | 28416 | 52.4 | 47.6 | 4276 | 28672 | 28653 | 51.8 | 55 | 0.5 | 277 | 78.6 | 51.6 | 36 | 55.7 |
| 757 | 1777 | 28396 | 28416 | 52.4 | 47.6 | 4277 | 28671 | 28652 | 52.8 | 55 | 0.4 | 276 | 78.6 | 51.4 | 36 | 55.8 |
| 758 | 1778 | 3229 | 3248 | 50.6 | 50 | 4278 | 3647 | 3628 | 50.6 | 45 | 0 | 419 | 75.3 | 41.5 | 36 | 53 |
| 759 | 1779 | 12235 | 12253 | 50.1 | 52.6 | 4279 | 12992 | 12974 | 51.2 | 52.6 | 1.1 | 758 | 76.5 | 42.6 | 36 | 53.7 |
| 760 | 1780 | 3229 | 3248 | 50.6 | 50 | 4280 | 3646 | 3625 | 52 | 40.9 | 1.4 | 418 | 75.3 | 41.4 | 36 | 53 |
| 761 | 1781 | 3228 | 3248 | 52 | 47.6 | 4281 | 3650 | 3631 | 53.1 | 50 | 1.1 | 423 | 75.4 | 41.6 | 36 | 53.5 |
| 762 | 1782 | 3230 | 3249 | 50.1 | 45 | 4282 | 3647 | 3628 | 50.6 | 45 | 0.5 | 418 | 75.3 | 41.4 | 36 | 52.8 |
| 763 | 1783 | 9931 | 9950 | 50.2 | 45 | 4283 | 10449 | 10428 | 51.9 | 40.9 | 1.8 | 519 | 75.3 | 40.8 | 36 | 52.9 |
| 764 | 1784 | 1402 | 1422 | 50.2 | 42.9 | 4284 | 1622 | 1602 | 51.6 | 47.6 | 1.4 | 221 | 76.5 | 48 | 36 | 53.7 |
| 765 | 1785 | 9922 | 9941 | 51.3 | 50 | 4285 | 10608 | 10589 | 51 | 50 | 0.3 | 687 | 75.8 | 41.2 | 36 | 53.5 |
| 766 | 1786 | 3792 | 3810 | 52.9 | 52.6 | 4286 | 4318 | 4294 | 54.4 | 40 | 1.5 | 527 | 75.5 | 41.2 | 36 | 53.8 |
| 767 | 1787 | 2429 | 2447 | 50.2 | 47.4 | 4287 | 3189 | 3168 | 51 | 45.5 | 0.7 | 761 | 76.6 | 43 | 36 | 53.8 |
| 768 | 1788 | 18008 | 18029 | 54.5 | 50 | 4288 | 18443 | 18424 | 55.9 | 55 | 1.4 | 436 | 76 | 43.1 | 36 | 54.7 |
| 769 | 1789 | 13039 | 13058 | 51.8 | 50 | 4289 | 13179 | 13158 | 50.4 | 40.9 | 1.5 | 141 | 74 | 46.1 | 36 | 52 |
| 770 | 1790 | 942 | 961 | 52.8 | 50 | 4290 | 1484 | 1466 | 53.1 | 52.6 | 0.4 | 543 | 76.9 | 44.4 | 36 | 54.7 |
| 771 | 1791 | 943 | 961 | 50.3 | 47.4 | 4291 | 1483 | 1464 | 51.3 | 45 | 1 | 541 | 76.8 | 44.2 | 36 | 53.9 |
| 772 | 1792 | 28867 | 28886 | 53.2 | 50 | 4292 | 29358 | 29339 | 52.8 | 50 | 0.3 | 492 | 76.9 | 44.9 | 36 | 54.8 |
| 773 | 1793 | 943 | 961 | 50.3 | 47.4 | 4293 | 1483 | 1465 | 50.5 | 47.4 | 0.3 | 541 | 76.8 | 44.2 | 36 | 53.9 |
| 774 | 1794 | 28866 | 28886 | 55.4 | 52.4 | 4294 | 29301 | 29282 | 55.3 | 55 | 0.2 | 436 | 77 | 45.4 | 36 | 55.6 |
| 775 | 1795 | 12352 | 12375 | 52.9 | 41.7 | 4295 | 12997 | 12977 | 51.8 | 42.9 | 1.1 | 646 | 76.4 | 42.9 | 36 | 54.1 |
| 776 | 1796 | 28867 | 28887 | 53.7 | 47.6 | 4296 | 29358 | 29339 | 52.8 | 50 | 0.9 | 492 | 76.9 | 44.9 | 36 | 54.8 |
| 777 | 1797 | 3896 | 3917 | 50.7 | 40.9 | 4297 | 4608 | 4590 | 51.5 | 52.6 | 0.9 | 713 | 75.5 | 40.4 | 36 | 53.2 |
| 778 | 1798 | 6098 | 6118 | 50.3 | 42.9 | 4298 | 6486 | 6467 | 50.8 | 45 | 0.5 | 389 | 74.6 | 40.1 | 36 | 52.4 |
| 779 | 1799 | 28868 | 28888 | 51.4 | 42.9 | 4299 | 29358 | 29339 | 52.8 | 50 | 1.4 | 491 | 76.9 | 44.8 | 36 | 54.3 |
| 780 | 1800 | 8220 | 8240 | 54 | 47.6 | 4300 | 8931 | 8913 | 55.5 | 52.6 | 1.4 | 712 | 75.4 | 40 | 36 | 54.1 |
| 781 | 1801 | 2220 | 2239 | 51.3 | 45 | 4301 | 2672 | 2653 | 51.6 | 50 | 0.4 | 453 | 77 | 45.3 | 36 | 54.4 |
| 782 | 1802 | 12040 | 12057 | 50.6 | 50 | 4302 | 12493 | 12476 | 50.7 | 50 | 0.1 | 454 | 76.3 | 43.6 | 36 | 53.7 |
| 783 | 1803 | 942 | 960 | 52.1 | 52.6 | 4303 | 1483 | 1464 | 51.3 | 45 | 0.8 | 542 | 76.8 | 44.3 | 36 | 54.3 |
| 784 | 1804 | 28868 | 28889 | 52 | 40.9 | 4304 | 29358 | 29339 | 52.8 | 50 | 0.8 | 491 | 76.9 | 44.8 | 36 | 54.5 |
| 785 | 1805 | 942 | 960 | 52.1 | 52.6 | 4305 | 1483 | 1465 | 50.5 | 47.4 | 1.6 | 542 | 76.8 | 44.3 | 36 | 54 |
| 786 | 1806 | 12040 | 12057 | 50.6 | 50 | 4306 | 12724 | 12705 | 52.4 | 55 | 1.8 | 685 | 76.6 | 43.1 | 36 | 53.9 |
| 787 | 1807 | 942 | 960 | 52.1 | 52.6 | 4307 | 1484 | 1466 | 53.1 | 52.6 | 1 | 543 | 76.9 | 44.4 | 36 | 54.5 |
| 788 | 1808 | 11545 | 11563 | 50.8 | 47.4 | 4308 | 12253 | 12235 | 50.1 | 52.6 | 0.7 | 709 | 76.2 | 42.2 | 36 | 53.5 |
| 789 | 1809 | 98 | 118 | 50.6 | 42.9 | 4309 | 269 | 251 | 51.1 | 52.6 | 0.5 | 172 | 75 | 46.5 | 36 | 52.8 |
| 790 | 1810 | 12373 | 12391 | 50.8 | 47.4 | 4310 | 12911 | 12892 | 50.5 | 50 | 0.3 | 539 | 76.1 | 42.5 | 36 | 53.5 |
| 791 | 1811 | 16366 | 16384 | 50.3 | 52.6 | 4311 | 16781 | 16761 | 51.3 | 47.6 | 0.9 | 416 | 75.1 | 41.1 | 36 | 52.8 |
| 792 | 1812 | 9929 | 9946 | 50 | 50 | 4312 | 10183 | 10165 | 51.7 | 47.4 | 1.6 | 255 | 75.3 | 43.9 | 36 | 52.8 |
| 793 | 1813 | 12236 | 12256 | 51.2 | 42.9 | 4313 | 13000 | 12981 | 51.1 | 45 | 0.1 | 765 | 76.4 | 42.5 | 36 | 53.9 |
| 794 | 1814 | 3231 | 3252 | 52.7 | 45.5 | 4314 | 3650 | 3631 | 53.1 | 50 | 0.4 | 420 | 75.4 | 41.7 | 36 | 53.7 |
| 795 | 1815 | 11541 | 11560 | 50.1 | 45 | 4315 | 11727 | 11708 | 50.4 | 45 | 0.3 | 187 | 73 | 40.6 | 36 | 51.2 |
| 796 | 1816 | 3232 | 3252 | 51.1 | 47.6 | 4316 | 3494 | 3473 | 50.4 | 40.9 | 0.7 | 263 | 74.3 | 41.4 | 36 | 52.3 |
| 797 | 1817 | 7725 | 7743 | 50.8 | 47.4 | 4317 | 8054 | 8035 | 50.4 | 50 | 0.4 | 330 | 75 | 41.8 | 36 | 52.7 |
| 798 | 1818 | 28968 | 28988 | 50.9 | 47.6 | 4318 | 29358 | 29339 | 52.8 | 50 | 2 | 391 | 76.4 | 44.5 | 36 | 53.9 |
| 799 | 1819 | 11545 | 11563 | 50.8 | 47.4 | 4319 | 12257 | 12237 | 51.3 | 47.6 | 0.5 | 713 | 76.2 | 42.1 | 36 | 53.7 |
| 800 | 1820 | 24417 | 24436 | 52.6 | 50 | 4320 | 25080 | 25062 | 53.5 | 52.6 | 0.9 | 664 | 75.8 | 41.3 | 36 | 53.9 |
| 801 | 1821 | 28968 | 28989 | 51.5 | 45.5 | 4321 | 29358 | 29339 | 52.8 | 50 | 1.3 | 391 | 76.4 | 44.5 | 36 | 54.1 |
| 802 | 1822 | 3789 | 3808 | 53.5 | 50 | 4322 | 4318 | 4294 | 54.4 | 40 | 0.9 | 530 | 75.5 | 41.1 | 36 | 54 |
| 803 | 1823 | 3232 | 3252 | 51.1 | 47.6 | 4323 | 3646 | 3625 | 52 | 40.9 | 0.9 | 415 | 75.3 | 41.4 | 36 | 53.1 |
| 804 | 1824 | 3232 | 3252 | 51.1 | 47.6 | 4324 | 3647 | 3628 | 50.6 | 45 | 0.5 | 416 | 75.3 | 41.6 | 36 | 53 |
| 805 | 1825 | 28971 | 28993 | 51.9 | 43.5 | 4325 | 29306 | 29288 | 53.5 | 52.6 | 1.6 | 336 | 76.2 | 44.6 | 36 | 54 |
| 806 | 1826 | 24179 | 24200 | 53.3 | 40.9 | 4326 | 24818 | 24797 | 51.6 | 40.9 | 1.6 | 640 | 75.8 | 41.2 | 36 | 53.6 |
| 807 | 1827 | 3231 | 3251 | 52 | 47.6 | 4327 | 3650 | 3631 | 53.1 | 50 | 1.1 | 420 | 75.4 | 41.7 | 36 | 53.5 |
| 808 | 1828 | 9930 | 9950 | 52.6 | 47.6 | 4328 | 10449 | 10425 | 54.6 | 40 | 2 | 520 | 75.4 | 41 | 36 | 53.7 |
| 809 | 1829 | 8866 | 8885 | 51.1 | 45 | 4329 | 9254 | 9236 | 50.6 | 47.4 | 0.5 | 389 | 75 | 41.1 | 36 | 52.8 |
| 810 | 1830 | 2522 | 2541 | 51.4 | 45 | 4330 | 2672 | 2653 | 51.6 | 50 | 0.2 | 151 | 75.3 | 48.3 | 36 | 53.2 |
| 811 | 1831 | 11541 | 11561 | 50.9 | 42.9 | 4331 | 12258 | 12238 | 50.3 | 42.9 | 0.6 | 718 | 76.2 | 41.9 | 36 | 53.5 |
| 812 | 1832 | 3232 | 3251 | 50.3 | 50 | 4332 | 3646 | 3625 | 52 | 40.9 | 1.7 | 415 | 75.3 | 41.4 | 36 | 52.9 |
| 813 | 1833 | 3232 | 3251 | 50.3 | 50 | 4333 | 3647 | 3628 | 50.6 | 45 | 0.3 | 416 | 75.3 | 41.6 | 36 | 52.9 |
| 814 | 1834 | 23843 | 23863 | 50.3 | 42.9 | 4334 | 24527 | 24508 | 50.5 | 45 | 0.2 | 685 | 76 | 41.8 | 36 | 53.4 |
| 815 | 1835 | 21210 | 21228 | 53.2 | 52.6 | 4335 | 21317 | 21293 | 53.2 | 40 | 0 | 108 | 71.1 | 42.6 | 36 | 50.8 |
| 816 | 1836 | 3229 | 3249 | 51.4 | 47.6 | 4336 | 3650 | 3631 | 53.1 | 50 | 1.7 | 422 | 75.4 | 41.7 | 36 | 53.3 |
| 817 | 1837 | 3230 | 3249 | 50.1 | 45 | 4337 | 3494 | 3473 | 50.4 | 40.9 | 0.3 | 265 | 74.2 | 41.1 | 36 | 52.1 |
| 818 | 1838 | 2371 | 2389 | 50.3 | 47.4 | 4338 | 2997 | 2976 | 51.4 | 40.9 | 1.1 | 627 | 76.7 | 43.5 | 36 | 53.9 |
| 819 | 1839 | 29186 | 29206 | 51.3 | 42.9 | 4339 | 29298 | 29280 | 51.4 | 52.6 | 0.1 | 113 | 72.8 | 46 | 36 | 51.5 |
| 820 | 1840 | 9929 | 9946 | 50 | 50 | 4340 | 10455 | 10435 | 50.5 | 42.9 | 0.4 | 527 | 75.3 | 40.6 | 36 | 52.8 |
| 821 | 1841 | 9351 | 9370 | 51.2 | 50 | 4341 | 9989 | 9968 | 51 | 40.9 | 0.2 | 639 | 75.4 | 40.4 | 36 | 53.2 |
| 822 | 1842 | 25348 | 25365 | 50.4 | 50 | 4342 | 25772 | 25753 | 51.9 | 50 | 1.5 | 425 | 74.9 | 40.5 | 36 | 52.6 |
| 823 | 1843 | 1402 | 1422 | 50.2 | 42.9 | 4343 | 2103 | 2082 | 52 | 45.5 | 1.8 | 702 | 76.7 | 43.3 | 36 | 53.8 |
| 824 | 1844 | 9929 | 9946 | 50 | 50 | 4344 | 10608 | 10589 | 51 | 50 | 0.9 | 680 | 75.8 | 41.2 | 36 | 53.2 |
| 825 | 1845 | 9934 | 9953 | 50.7 | 50 | 4345 | 10608 | 10589 | 51 | 50 | 0.3 | 675 | 75.8 | 41.2 | 36 | 53.4 |
| 826 | 1846 | 13176 | 13196 | 51.4 | 47.6 | 4346 | 13544 | 13525 | 52.6 | 55 | 1.2 | 369 | 77.1 | 46.3 | 36 | 54.5 |
| 827 | 1847 | 7725 | 7743 | 50.8 | 47.4 | 4347 | 8189 | 8170 | 50.6 | 50 | 0.2 | 465 | 75.6 | 41.9 | 36 | 53.2 |
| 828 | 1848 | 7725 | 7743 | 50.8 | 47.4 | 4348 | 8190 | 8172 | 50.3 | 47.4 | 0.6 | 466 | 75.6 | 41.8 | 36 | 53.1 |
| 829 | 1849 | 18074 | 18093 | 50.3 | 45 | 4349 | 18662 | 18641 | 50.4 | 40.9 | 0.1 | 589 | 76.2 | 42.6 | 36 | 53.6 |
| 830 | 1850 | 18074 | 18093 | 50.3 | 45 | 4350 | 18632 | 18611 | 50.2 | 40.9 | 0.1 | 559 | 76.2 | 42.8 | 36 | 53.5 |
| 831 | 1851 | 29200 | 29222 | 53.2 | 43.5 | 4351 | 29306 | 29288 | 53.5 | 52.6 | 0.3 | 107 | 73.1 | 47.7 | 36 | 52.3 |
| 832 | 1852 | 25348 | 25366 | 51.2 | 47.4 | 4352 | 25545 | 25526 | 51.7 | 45 | 0.5 | 198 | 74.1 | 42.9 | 36 | 52.3 |
| 833 | 1853 | 25348 | 25366 | 51.2 | 47.4 | 4353 | 25545 | 25525 | 52.3 | 42.9 | 1.1 | 198 | 74.1 | 42.9 | 36 | 52.3 |
| 834 | 1854 | 29200 | 29223 | 53.7 | 41.7 | 4354 | 29306 | 29288 | 53.5 | 52.6 | 0.2 | 107 | 73.1 | 47.7 | 36 | 52.3 |
| 835 | 1855 | 25347 | 25366 | 52.7 | 50 | 4355 | 25545 | 25521 | 54.5 | 40 | 1.8 | 199 | 74.2 | 43.2 | 36 | 52.9 |
| 836 | 1856 | 3792 | 3811 | 54 | 55 | 4356 | 4447 | 4425 | 53 | 43.5 | 1 | 656 | 75.5 | 40.5 | 36 | 53.9 |
| 837 | 1857 | 29200 | 29224 | 54.2 | 40 | 4357 | 29306 | 29288 | 53.5 | 52.6 | 0.7 | 107 | 73.1 | 47.7 | 36 | 52.3 |
| 838 | 1858 | 985 | 1004 | 51.1 | 50 | 4358 | 1483 | 1465 | 50.5 | 47.4 | 0.6 | 499 | 76.4 | 43.5 | 36 | 53.7 |
| 839 | 1859 | 2427 | 2445 | 52.1 | 52.6 | 4359 | 3189 | 3168 | 51 | 45.5 | 1.1 | 763 | 76.7 | 43.1 | 36 | 54.1 |
| 840 | 1860 | 13701 | 13725 | 53.6 | 40 | 4360 | 14084 | 14060 | 53.6 | 40 | 0.1 | 384 | 74.6 | 40.1 | 36 | 53.4 |
| 841 | 1861 | 985 | 1004 | 51.1 | 50 | 4361 | 1483 | 1464 | 51.3 | 45 | 0.2 | 499 | 76.4 | 43.5 | 36 | 53.9 |
| 842 | 1862 | 8794 | 8813 | 51.6 | 45 | 4362 | 9559 | 9539 | 50.6 | 42.9 | 1 | 766 | 75.9 | 41.3 | 37 | 53.4 |
| 843 | 1863 | 3789 | 3806 | 50 | 50 | 4363 | 4435 | 4417 | 50.5 | 52.6 | 0.4 | 647 | 75.5 | 40.5 | 37 | 52.9 |
| 844 | 1864 | 13177 | 13197 | 50.3 | 42.9 | 4364 | 13314 | 13297 | 51 | 50 | 0.6 | 138 | 72.8 | 43.5 | 37 | 51.2 |
| 845 | 1865 | 3791 | 3808 | 50 | 50 | 4365 | 4435 | 4417 | 50.5 | 52.6 | 0.4 | 645 | 75.4 | 40.5 | 37 | 52.9 |
| 846 | 1866 | 9139 | 9159 | 52.5 | 47.6 | 4366 | 9364 | 9346 | 53.9 | 52.6 | 1.4 | 226 | 74.9 | 43.8 | 37 | 53.3 |
| 847 | 1867 | 3226 | 3245 | 51.7 | 55 | 4367 | 3494 | 3473 | 50.4 | 40.9 | 1.3 | 269 | 74.5 | 41.6 | 37 | 52.4 |
| 848 | 1868 | 13040 | 13059 | 50.9 | 50 | 4368 | 13314 | 13297 | 51 | 50 | 0.1 | 275 | 75.6 | 44.4 | 37 | 53.3 |
| 849 | 1869 | 2522 | 2541 | 51.4 | 45 | 4369 | 2891 | 2873 | 50.8 | 47.4 | 0.6 | 370 | 76 | 43.8 | 37 | 53.6 |
| 850 | 1870 | 8865 | 8884 | 50.4 | 45 | 4370 | 9245 | 9226 | 50 | 45 | 0.4 | 381 | 74.9 | 40.9 | 37 | 52.6 |
| 851 | 1871 | 3787 | 3804 | 50 | 50 | 4371 | 4434 | 4416 | 51.5 | 52.6 | 1.4 | 648 | 75.4 | 40.3 | 37 | 52.9 |
| 852 | 1872 | 3226 | 3245 | 51.7 | 55 | 4372 | 3646 | 3625 | 52 | 40.9 | 0.3 | 421 | 75.3 | 41.6 | 37 | 53.4 |
| 853 | 1873 | 3226 | 3245 | 51.7 | 55 | 4373 | 3647 | 3628 | 50.6 | 45 | 1.1 | 422 | 75.4 | 41.7 | 37 | 53.1 |
| 854 | 1874 | 3226 | 3245 | 51.7 | 55 | 4374 | 3650 | 3631 | 53.1 | 50 | 1.4 | 425 | 75.5 | 41.9 | 37 | 53.5 |
| 855 | 1875 | 2387 | 2405 | 51.6 | 52.6 | 4375 | 2747 | 2727 | 50 | 42.9 | 1.6 | 361 | 76.9 | 46 | 37 | 53.9 |
| 856 | 1876 | 18074 | 18093 | 50.3 | 45 | 4376 | 18229 | 18209 | 50.1 | 42.9 | 0.2 | 156 | 73.2 | 42.9 | 37 | 51.4 |
| 857 | 1877 | 13701 | 13725 | 53.6 | 40 | 4377 | 14059 | 14040 | 52.8 | 50 | 0.8 | 359 | 74.5 | 40.1 | 37 | 53.1 |
| 858 | 1878 | 3787 | 3804 | 50 | 50 | 4378 | 4435 | 4417 | 50.5 | 52.6 | 0.4 | 649 | 75.4 | 40.4 | 37 | 52.9 |
| 859 | 1879 | 13040 | 13059 | 50.9 | 50 | 4379 | 13323 | 13304 | 51.1 | 45 | 0.2 | 284 | 75.7 | 44.4 | 37 | 53.4 |
| 860 | 1880 | 3789 | 3806 | 50 | 50 | 4380 | 4434 | 4416 | 51.5 | 52.6 | 1.4 | 646 | 75.4 | 40.4 | 37 | 52.9 |
| 861 | 1881 | 15506 | 15527 | 50.8 | 40.9 | 4381 | 16214 | 16196 | 51.8 | 52.6 | 1 | 709 | 75.5 | 40.3 | 37 | 53.2 |
| 862 | 1882 | 12234 | 12252 | 50.6 | 47.4 | 4382 | 12412 | 12392 | 50 | 42.9 | 0.6 | 179 | 73.1 | 41.3 | 37 | 51.3 |
| 863 | 1883 | 12234 | 12252 | 50.6 | 47.4 | 4383 | 12739 | 12718 | 51 | 40.9 | 0.4 | 506 | 75.8 | 42.1 | 37 | 53.4 |
| 864 | 1884 | 18074 | 18094 | 51.1 | 42.9 | 4384 | 18229 | 18209 | 50.1 | 42.9 | 0.9 | 156 | 73.2 | 42.9 | 37 | 51.4 |
| 865 | 1885 | 18075 | 18095 | 50.6 | 47.6 | 4385 | 18223 | 18206 | 51.8 | 50 | 1.2 | 149 | 73.3 | 43.6 | 37 | 51.6 |
| 866 | 1886 | 13040 | 13059 | 50.9 | 50 | 4386 | 13326 | 13306 | 50.7 | 42.9 | 0.2 | 287 | 75.7 | 44.3 | 37 | 53.3 |
| 867 | 1887 | 18080 | 18098 | 51.2 | 52.6 | 4387 | 18233 | 18215 | 51.3 | 52.6 | 0.1 | 154 | 73.9 | 44.8 | 37 | 52.2 |
| 868 | 1888 | 18080 | 18098 | 51.2 | 52.6 | 4388 | 18233 | 18214 | 52 | 50 | 0.9 | 154 | 73.9 | 44.8 | 37 | 52.2 |
| 869 | 1889 | 18080 | 18098 | 51.2 | 52.6 | 4389 | 18231 | 18210 | 52.2 | 45.5 | 1 | 152 | 73.5 | 44.1 | 37 | 51.9 |
| 870 | 1890 | 18080 | 18098 | 51.2 | 52.6 | 4390 | 18223 | 18206 | 51.8 | 50 | 0.6 | 144 | 73.2 | 43.8 | 37 | 51.7 |
| 871 | 1891 | 18077 | 18098 | 52.9 | 50 | 4391 | 18220 | 18202 | 54.8 | 52.6 | 1.9 | 144 | 73.2 | 43.8 | 37 | 52.2 |
| 872 | 1892 | 18076 | 18098 | 54.4 | 47.8 | 4392 | 18443 | 18424 | 55.9 | 55 | 1.5 | 368 | 75.8 | 43.2 | 37 | 54.5 |
| 873 | 1893 | 3792 | 3810 | 52.9 | 52.6 | 4393 | 4436 | 4417 | 52.2 | 50 | 0.6 | 645 | 75.4 | 40.5 | 37 | 53.6 |
| 874 | 1894 | 3055 | 3074 | 51.1 | 50 | 4394 | 3647 | 3628 | 50.6 | 45 | 0.5 | 593 | 76.3 | 42.7 | 37 | 53.7 |
| 875 | 1895 | 3055 | 3074 | 51.1 | 50 | 4395 | 3646 | 3625 | 52 | 40.9 | 0.9 | 592 | 76.2 | 42.6 | 37 | 53.8 |
| 876 | 1896 | 15506 | 15527 | 50.8 | 40.9 | 4396 | 15645 | 15625 | 51.1 | 42.9 | 0.4 | 140 | 71.8 | 40.7 | 37 | 50.6 |
| 877 | 1897 | 18081 | 18099 | 51.2 | 52.6 | 4397 | 18229 | 18209 | 50.1 | 42.9 | 1.1 | 149 | 73.3 | 43.6 | 37 | 51.4 |
| 878 | 1898 | 13039 | 13058 | 51.8 | 50 | 4398 | 13155 | 13138 | 50.4 | 50 | 1.4 | 117 | 73.4 | 47 | 37 | 51.6 |
| 879 | 1899 | 3055 | 3075 | 51.8 | 47.6 | 4399 | 3650 | 3631 | 53.1 | 50 | 1.3 | 596 | 76.3 | 42.8 | 37 | 54.1 |
| 880 | 1900 | 18080 | 18099 | 53 | 50 | 4400 | 18223 | 18205 | 53.3 | 52.6 | 0.4 | 144 | 73.2 | 43.8 | 37 | 52.2 |
| 881 | 1901 | 27361 | 27380 | 52.4 | 55 | 4401 | 27579 | 27558 | 51.1 | 40.9 | 1.3 | 219 | 75.4 | 45.2 | 37 | 53.2 |
| 882 | 1902 | 3221 | 3239 | 51.5 | 52.6 | 4402 | 3503 | 3484 | 51.5 | 50 | 0 | 283 | 74.8 | 42 | 37 | 52.9 |
| 883 | 1903 | 3221 | 3239 | 51.5 | 52.6 | 4403 | 3504 | 3485 | 50.4 | 45 | 1.1 | 284 | 74.7 | 41.9 | 37 | 52.5 |
| 884 | 1904 | 18077 | 18099 | 54.4 | 47.8 | 4404 | 18220 | 18201 | 56.1 | 55 | 1.7 | 144 | 73.2 | 43.8 | 37 | 52.6 |
| 885 | 1905 | 3055 | 3075 | 51.8 | 47.6 | 4405 | 3647 | 3628 | 50.6 | 45 | 1.2 | 593 | 76.3 | 42.7 | 37 | 53.7 |
| 886 | 1906 | 18581 | 18599 | 51.4 | 47.4 | 4406 | 18697 | 18679 | 51.9 | 52.6 | 0.4 | 117 | 71 | 41 | 37 | 50.2 |
| 887 | 1907 | 18616 | 18636 | 51.4 | 47.6 | 4407 | 19216 | 19195 | 50.2 | 40.9 | 1.1 | 601 | 75.6 | 41.1 | 37 | 53.1 |
| 888 | 1908 | 3219 | 3238 | 50.7 | 50 | 4408 | 3503 | 3484 | 51.5 | 50 | 0.8 | 285 | 74.8 | 42.1 | 37 | 52.7 |
| 889 | 1909 | 18696 | 18715 | 51.7 | 50 | 4409 | 19216 | 19195 | 50.2 | 40.9 | 1.5 | 521 | 75.5 | 41.3 | 37 | 53 |
| 890 | 1910 | 3219 | 3238 | 50.7 | 50 | 4410 | 3504 | 3485 | 50.4 | 45 | 0.3 | 286 | 74.7 | 42 | 37 | 52.5 |
| 891 | 1911 | 27366 | 27384 | 52.2 | 52.6 | 4411 | 27573 | 27552 | 52.3 | 40.9 | 0.1 | 208 | 74.6 | 43.8 | 37 | 53 |
| 892 | 1912 | 27366 | 27384 | 52.2 | 52.6 | 4412 | 27567 | 27547 | 51.1 | 42.9 | 1 | 202 | 74.6 | 44.1 | 37 | 52.7 |
| 893 | 1913 | 3055 | 3075 | 51.8 | 47.6 | 4413 | 3646 | 3625 | 52 | 40.9 | 0.2 | 592 | 76.2 | 42.6 | 37 | 54 |
| 894 | 1914 | 18704 | 18724 | 50.8 | 47.6 | 4414 | 19216 | 19195 | 50.2 | 40.9 | 0.5 | 513 | 75.5 | 41.1 | 37 | 53 |
| 895 | 1915 | 16874 | 16893 | 52.1 | 50 | 4415 | 17056 | 17035 | 51.8 | 45.5 | 0.4 | 183 | 74.4 | 44.3 | 37 | 52.7 |
| 896 | 1916 | 12234 | 12252 | 50.6 | 47.4 | 4416 | 12739 | 12719 | 50.3 | 42.9 | 0.3 | 506 | 75.8 | 42.1 | 37 | 53.3 |
| 897 | 1917 | 7728 | 7746 | 51.7 | 52.6 | 4417 | 8054 | 8035 | 50.4 | 50 | 1.2 | 327 | 75 | 41.9 | 37 | 52.7 |
| 898 | 1918 | 15506 | 15527 | 50.8 | 40.9 | 4418 | 15647 | 15628 | 51 | 45 | 0.3 | 142 | 71.9 | 40.8 | 37 | 50.7 |
| 899 | 1919 | 985 | 1004 | 51.1 | 50 | 4419 | 1773 | 1755 | 51.7 | 52.6 | 0.6 | 789 | 76.7 | 43.1 | 37 | 54.1 |
| 900 | 1920 | 3217 | 3236 | 51.1 | 50 | 4420 | 3503 | 3484 | 51.5 | 50 | 0.4 | 287 | 74.8 | 42.2 | 37 | 52.8 |
| 901 | 1921 | 3791 | 3808 | 50 | 50 | 4421 | 4434 | 4416 | 51.5 | 52.6 | 1.4 | 644 | 75.4 | 40.4 | 37 | 52.9 |
| 902 | 1922 | 19794 | 19813 | 50 | 50 | 4422 | 19923 | 19904 | 50.1 | 50 | 0.1 | 130 | 72.7 | 43.8 | 37 | 51 |
| 903 | 1923 | 13039 | 13058 | 51.8 | 50 | 4423 | 13178 | 13157 | 50.4 | 40.9 | 1.5 | 140 | 73.8 | 45.7 | 37 | 51.9 |
| 904 | 1924 | 13033 | 13051 | 52.1 | 52.6 | 4424 | 13155 | 13138 | 50.4 | 50 | 1.7 | 123 | 73.7 | 47.2 | 37 | 51.8 |
| 905 | 1925 | 12233 | 12251 | 51.1 | 52.6 | 4425 | 12739 | 12719 | 50.3 | 42.9 | 0.9 | 507 | 75.9 | 42.2 | 37 | 53.3 |
| 906 | 1926 | 19795 | 19814 | 50.4 | 45 | 4426 | 19923 | 19903 | 50.9 | 47.6 | 0.4 | 129 | 72.5 | 43.4 | 37 | 51 |
| 907 | 1927 | 13177 | 13197 | 50.3 | 42.9 | 4427 | 13946 | 13929 | 51.5 | 50 | 1.2 | 770 | 75.9 | 41 | 37 | 53.3 |
| 908 | 1928 | 3799 | 3820 | 52.9 | 45.5 | 4428 | 4318 | 4294 | 54.4 | 40 | 1.5 | 520 | 75.4 | 41 | 37 | 53.7 |
| 909 | 1929 | 8867 | 8887 | 52.3 | 47.6 | 4429 | 9364 | 9346 | 53.9 | 52.6 | 1.6 | 498 | 75.8 | 42.2 | 37 | 53.9 |
| 910 | 1930 | 1472 | 1491 | 51.2 | 45 | 4430 | 2152 | 2133 | 50.7 | 45 | 0.5 | 681 | 76.5 | 42.9 | 37 | 53.8 |
| 911 | 1931 | 12233 | 12251 | 51.1 | 52.6 | 4431 | 12739 | 12718 | 51 | 40.9 | 0.2 | 507 | 75.9 | 42.2 | 37 | 53.5 |
| 912 | 1932 | 3055 | 3076 | 52.4 | 45.5 | 4432 | 3650 | 3631 | 53.1 | 50 | 0.7 | 596 | 76.3 | 42.8 | 37 | 54.2 |
| 913 | 1933 | 12726 | 12746 | 51.3 | 47.6 | 4433 | 13325 | 13305 | 50.5 | 47.6 | 0.7 | 600 | 76.7 | 43.7 | 37 | 53.9 |
| 914 | 1934 | 8867 | 8887 | 52.3 | 47.6 | 4434 | 9316 | 9296 | 50.8 | 42.9 | 1.5 | 450 | 75.4 | 41.6 | 37 | 53.1 |
| 915 | 1935 | 8867 | 8887 | 52.3 | 47.6 | 4435 | 9314 | 9295 | 51.1 | 50 | 1.2 | 448 | 75.5 | 41.7 | 37 | 53.3 |
| 916 | 1936 | 8867 | 8887 | 52.3 | 47.6 | 4436 | 9313 | 9294 | 50.4 | 50 | 1.9 | 447 | 75.5 | 41.6 | 37 | 53 |
| 917 | 1937 | 3055 | 3076 | 52.4 | 45.5 | 4437 | 3647 | 3628 | 50.6 | 45 | 1.8 | 593 | 76.3 | 42.7 | 37 | 53.7 |
| 918 | 1938 | 13176 | 13196 | 51.4 | 47.6 | 4438 | 13312 | 13294 | 51 | 52.6 | 0.4 | 137 | 72.9 | 43.8 | 37 | 51.5 |
| 919 | 1939 | 12726 | 12746 | 51.3 | 47.6 | 4439 | 13155 | 13138 | 50.4 | 50 | 0.9 | 430 | 76.4 | 44 | 37 | 53.7 |
| 920 | 1940 | 13701 | 13724 | 53.1 | 41.7 | 4440 | 14058 | 14040 | 51.4 | 52.6 | 1.7 | 358 | 74.5 | 40.2 | 37 | 52.7 |
| 921 | 1941 | 8372 | 8390 | 50.7 | 47.4 | 4441 | 9101 | 9081 | 50.5 | 47.6 | 0.2 | 730 | 75.5 | 40.3 | 37 | 53.1 |
| 922 | 1942 | 3055 | 3076 | 52.4 | 45.5 | 4442 | 3646 | 3625 | 52 | 40.9 | 0.4 | 592 | 76.2 | 42.6 | 37 | 54.1 |
| 923 | 1943 | 887 | 905 | 50.1 | 47.4 | 4443 | 1493 | 1474 | 50.8 | 45 | 0.7 | 607 | 77.1 | 44.6 | 37 | 54.1 |
| 924 | 1944 | 1046 | 1063 | 50.3 | 50 | 4444 | 1697 | 1677 | 51 | 42.9 | 0.7 | 652 | 76.9 | 43.9 | 37 | 54 |
| 925 | 1945 | 27378 | 27397 | 50.5 | 45 | 4445 | 27675 | 27656 | 50 | 40 | 0.5 | 298 | 74.1 | 40.3 | 37 | 52 |
| 926 | 1946 | 27378 | 27397 | 50.5 | 45 | 4446 | 27674 | 27654 | 51.9 | 42.9 | 1.4 | 297 | 74.2 | 40.4 | 37 | 52.2 |
| 927 | 1947 | 2671 | 2692 | 52.1 | 40.9 | 4447 | 3056 | 3037 | 52.1 | 55 | 0 | 386 | 74.8 | 40.7 | 37 | 53.1 |
| 928 | 1948 | 1046 | 1063 | 50.3 | 50 | 4448 | 1697 | 1678 | 50.3 | 45 | 0.1 | 652 | 76.9 | 43.9 | 37 | 54 |
| 929 | 1949 | 2387 | 2405 | 51.6 | 52.6 | 4449 | 2672 | 2654 | 50.9 | 52.6 | 0.8 | 286 | 77 | 47.6 | 37 | 54.3 |
| 930 | 1950 | 3792 | 3810 | 52.9 | 52.6 | 4450 | 4565 | 4542 | 53.9 | 41.7 | 1 | 774 | 75.6 | 40.3 | 37 | 53.8 |
| 931 | 1951 | 15506 | 15527 | 50.8 | 40.9 | 4451 | 15647 | 15629 | 50.3 | 47.4 | 0.5 | 142 | 71.9 | 40.8 | 37 | 50.5 |
| 932 | 1952 | 8794 | 8813 | 51.6 | 45 | 4452 | 9560 | 9540 | 51.6 | 42.9 | 0 | 767 | 75.9 | 41.2 | 37 | 53.7 |
| 933 | 1953 | 19801 | 19819 | 53.2 | 52.6 | 4453 | 19909 | 19885 | 52.5 | 40 | 0.7 | 109 | 71.4 | 43.1 | 37 | 50.8 |
| 934 | 1954 | 19988 | 20006 | 50.4 | 47.4 | 4454 | 20615 | 20597 | 50.6 | 47.4 | 0.2 | 628 | 75.3 | 40.1 | 37 | 52.9 |
| 935 | 1955 | 19991 | 20009 | 52.8 | 52.6 | 4455 | 20616 | 20597 | 52.3 | 45 | 0.5 | 626 | 75.3 | 40.1 | 37 | 53.5 |
| 936 | 1956 | 16875 | 16895 | 51.6 | 47.6 | 4456 | 17060 | 17041 | 51.1 | 50 | 0.5 | 186 | 74.6 | 44.6 | 37 | 52.6 |
| 937 | 1957 | 16875 | 16895 | 51.6 | 47.6 | 4457 | 17059 | 17039 | 50.6 | 47.6 | 1 | 185 | 74.4 | 44.3 | 37 | 52.4 |
| 938 | 1958 | 16875 | 16895 | 51.6 | 47.6 | 4458 | 17056 | 17035 | 51.8 | 45.5 | 0.2 | 182 | 74.2 | 44 | 37 | 52.5 |
| 939 | 1959 | 27442 | 27461 | 51.5 | 40 | 4459 | 27541 | 27521 | 51.7 | 47.6 | 0.1 | 100 | 71.2 | 44 | 37 | 50.4 |
| 940 | 1960 | 16875 | 16896 | 52.2 | 45.5 | 4460 | 17060 | 17041 | 51.1 | 50 | 1.1 | 186 | 74.6 | 44.6 | 37 | 52.6 |
| 941 | 1961 | 23841 | 23859 | 50.5 | 52.6 | 4461 | 24527 | 24507 | 51 | 42.9 | 0.5 | 687 | 76.1 | 41.9 | 37 | 53.5 |
| 942 | 1962 | 23841 | 23859 | 50.5 | 52.6 | 4462 | 24093 | 24075 | 50.9 | 52.6 | 0.4 | 253 | 76 | 45.8 | 37 | 53.5 |
| 943 | 1963 | 16875 | 16896 | 52.2 | 45.5 | 4463 | 17059 | 17039 | 50.6 | 47.6 | 1.6 | 185 | 74.4 | 44.3 | 37 | 52.4 |
| 944 | 1964 | 16875 | 16896 | 52.2 | 45.5 | 4464 | 17056 | 17035 | 51.8 | 45.5 | 0.5 | 182 | 74.2 | 44 | 37 | 52.6 |
| 945 | 1965 | 23843 | 23863 | 50.3 | 42.9 | 4465 | 24093 | 24075 | 50.9 | 52.6 | 0.5 | 251 | 75.8 | 45.4 | 37 | 53.3 |
| 946 | 1966 | 16875 | 16896 | 52.2 | 45.5 | 4466 | 17041 | 17023 | 53.5 | 52.6 | 1.3 | 167 | 73.8 | 43.7 | 37 | 52.4 |
| 947 | 1967 | 28187 | 28205 | 53.1 | 52.6 | 4467 | 28673 | 28654 | 53.5 | 55 | 0.5 | 487 | 80 | 52.4 | 37 | 57 |
| 948 | 1968 | 28190 | 28208 | 51.7 | 52.6 | 4468 | 28672 | 28654 | 50.6 | 52.6 | 1.2 | 483 | 79.9 | 52.2 | 37 | 56.2 |
| 949 | 1969 | 24030 | 24047 | 50.7 | 50 | 4469 | 24527 | 24508 | 50.5 | 45 | 0.2 | 498 | 75.4 | 41.2 | 37 | 53 |
| 950 | 1970 | 24031 | 24050 | 56.5 | 55 | 4470 | 24816 | 24792 | 54.7 | 40 | 1.8 | 786 | 76.3 | 42 | 37 | 54.9 |
| 951 | 1971 | 7880 | 7900 | 50.3 | 42.9 | 4471 | 8049 | 8032 | 50.4 | 50 | 0 | 170 | 72.8 | 41.2 | 37 | 51.2 |
| 952 | 1972 | 24096 | 24119 | 54.4 | 41.7 | 4472 | 24815 | 24791 | 54.5 | 40 | 0.1 | 720 | 75.8 | 41 | 37 | 54.5 |
| 953 | 1973 | 17790 | 17811 | 51.6 | 40.9 | 4473 | 18233 | 18214 | 52 | 50 | 0.4 | 444 | 75.1 | 40.8 | 37 | 53.2 |
| 954 | 1974 | 24174 | 24194 | 50.9 | 42.9 | 4474 | 24938 | 24921 | 50.4 | 50 | 0.5 | 765 | 75.8 | 40.9 | 37 | 53.3 |
| 955 | 1975 | 16875 | 16896 | 52.2 | 45.5 | 4475 | 17039 | 17022 | 51.4 | 50 | 0.8 | 165 | 73.7 | 43.6 | 37 | 52.1 |
| 956 | 1976 | 24174 | 24195 | 52.5 | 40.9 | 4476 | 24936 | 24919 | 51.8 | 50 | 0.7 | 763 | 75.8 | 41 | 37 | 53.7 |
| 957 | 1977 | 24179 | 24198 | 51 | 45 | 4477 | 24938 | 24921 | 50.4 | 50 | 0.6 | 760 | 75.8 | 40.9 | 37 | 53.3 |
| 958 | 1978 | 16875 | 16896 | 52.2 | 45.5 | 4478 | 17038 | 17021 | 50.7 | 50 | 1.6 | 164 | 73.8 | 43.9 | 37 | 52 |
| 959 | 1979 | 24180 | 24199 | 50.3 | 40 | 4479 | 24936 | 24919 | 51.8 | 50 | 1.5 | 757 | 75.8 | 41 | 37 | 53.2 |
| 960 | 1980 | 2823 | 2844 | 50.4 | 45.5 | 4480 | 3186 | 3165 | 50.4 | 40.9 | 0 | 364 | 75.5 | 42.6 | 37 | 53.1 |
| 961 | 1981 | 10142 | 10163 | 51.3 | 40.9 | 4481 | 10608 | 10589 | 51 | 50 | 0.3 | 467 | 74.9 | 40 | 37 | 52.8 |
| 962 | 1982 | 1046 | 1063 | 50.3 | 50 | 4482 | 1483 | 1464 | 51.3 | 45 | 0.9 | 438 | 76.2 | 43.6 | 37 | 53.6 |
| 963 | 1983 | 17388 | 17408 | 50.7 | 42.9 | 4483 | 17501 | 17481 | 51.2 | 42.9 | 0.6 | 114 | 70.5 | 40.4 | 37 | 49.7 |
| 964 | 1984 | 24179 | 24200 | 53.3 | 40.9 | 4484 | 24740 | 24717 | 52.5 | 41.7 | 0.8 | 562 | 76 | 42.2 | 37 | 54 |
| 965 | 1985 | 24379 | 24398 | 55 | 55 | 4485 | 25088 | 25070 | 54.5 | 52.6 | 0.5 | 710 | 75.9 | 41.3 | 37 | 54.6 |
| 966 | 1986 | 24379 | 24398 | 55 | 55 | 4486 | 25087 | 25069 | 53.7 | 52.6 | 1.3 | 709 | 75.9 | 41.3 | 37 | 54.3 |
| 967 | 1987 | 16874 | 16893 | 52.1 | 50 | 4487 | 17059 | 17039 | 50.6 | 47.6 | 1.5 | 186 | 74.6 | 44.6 | 37 | 52.5 |
| 968 | 1988 | 24380 | 24399 | 55 | 55 | 4488 | 25088 | 25070 | 54.5 | 52.6 | 0.5 | 709 | 75.9 | 41.3 | 37 | 54.6 |
| 969 | 1989 | 28522 | 28542 | 50.2 | 42.9 | 4489 | 28671 | 28653 | 50.2 | 52.6 | 0 | 150 | 76.2 | 50.7 | 37 | 53.5 |
| 970 | 1990 | 16874 | 16893 | 52.1 | 50 | 4490 | 17060 | 17041 | 51.1 | 50 | 1 | 187 | 74.7 | 44.9 | 37 | 52.7 |
| 971 | 1991 | 24380 | 24399 | 55 | 55 | 4491 | 25087 | 25069 | 53.7 | 52.6 | 1.3 | 708 | 75.9 | 41.4 | 37 | 54.4 |
| 972 | 1992 | 17608 | 17627 | 50.2 | 45 | 4492 | 18239 | 18220 | 50 | 45 | 0.2 | 632 | 75.3 | 40.2 | 37 | 52.8 |
| 973 | 1993 | 17608 | 17627 | 50.2 | 45 | 4493 | 18238 | 18219 | 50.3 | 45 | 0.1 | 631 | 75.3 | 40.3 | 37 | 52.9 |
| 974 | 1994 | 1046 | 1063 | 50.3 | 50 | 4494 | 1483 | 1465 | 50.5 | 47.4 | 0.2 | 438 | 76.2 | 43.6 | 37 | 53.6 |
| 975 | 1995 | 17608 | 17628 | 50.9 | 42.9 | 4495 | 18239 | 18220 | 50 | 45 | 0.9 | 632 | 75.3 | 40.2 | 37 | 52.8 |
| 976 | 1996 | 17608 | 17628 | 50.9 | 42.9 | 4496 | 18238 | 18219 | 50.3 | 45 | 0.7 | 631 | 75.3 | 40.3 | 37 | 52.9 |
| 977 | 1997 | 8063 | 8084 | 51.4 | 45.5 | 4497 | 8188 | 8169 | 50.5 | 45 | 0.9 | 126 | 72.1 | 42.9 | 37 | 50.7 |
| 978 | 1998 | 12236 | 12256 | 51.2 | 42.9 | 4498 | 12739 | 12718 | 51 | 40.9 | 0.2 | 504 | 75.8 | 42.1 | 37 | 53.5 |
| 979 | 1999 | 13176 | 13196 | 51.4 | 47.6 | 4499 | 13325 | 13305 | 50.5 | 47.6 | 0.8 | 150 | 73.4 | 44 | 37 | 51.7 |
| 980 | 2000 | 2371 | 2389 | 50.3 | 47.4 | 4500 | 2749 | 2728 | 50.3 | 45.5 | 0 | 379 | 76.9 | 45.9 | 37 | 54.1 |
| 981 | 2001 | 9402 | 9420 | 51.3 | 47.4 | 4501 | 9989 | 9968 | 51 | 40.9 | 0.4 | 588 | 75.4 | 40.5 | 37 | 53.1 |
| 982 | 2002 | 9931 | 9950 | 50.2 | 45 | 4502 | 10183 | 10166 | 50.9 | 50 | 0.7 | 253 | 75.2 | 43.9 | 37 | 52.8 |
| 983 | 2003 | 2387 | 2405 | 51.6 | 52.6 | 4503 | 2997 | 2976 | 51.4 | 40.9 | 0.2 | 611 | 76.6 | 43.5 | 37 | 54.2 |
| 984 | 2004 | 3788 | 3805 | 50 | 50 | 4504 | 4435 | 4417 | 50.5 | 52.6 | 0.4 | 648 | 75.4 | 40.4 | 37 | 52.9 |
| 985 | 2005 | 26039 | 26057 | 52.6 | 52.6 | 4505 | 26650 | 26630 | 51.4 | 42.9 | 1.2 | 612 | 75.3 | 40.4 | 37 | 53.3 |
| 986 | 2006 | 2371 | 2389 | 50.3 | 47.4 | 4506 | 3053 | 3034 | 50.3 | 50 | 0 | 683 | 76.7 | 43.3 | 37 | 53.9 |
| 987 | 2007 | 3 | 21 | 53.4 | 52.6 | 4507 | 315 | 296 | 51.9 | 50 | 1.5 | 313 | 76.9 | 46.6 | 37 | 54.5 |
| 988 | 2008 | 2371 | 2389 | 50.3 | 47.4 | 4508 | 3056 | 3037 | 52.1 | 55 | 1.7 | 686 | 76.7 | 43.4 | 37 | 53.9 |
| 989 | 2009 | 13040 | 13059 | 50.9 | 50 | 4509 | 13155 | 13137 | 52.1 | 52.6 | 1.2 | 116 | 73.2 | 46.6 | 37 | 51.6 |
| 990 | 2010 | 9931 | 9950 | 50.2 | 45 | 4510 | 10183 | 10165 | 51.7 | 47.4 | 1.5 | 253 | 75.2 | 43.9 | 37 | 52.8 |
| 991 | 2011 | 3788 | 3805 | 50 | 50 | 4511 | 4434 | 4416 | 51.5 | 52.6 | 1.4 | 647 | 75.4 | 40.3 | 37 | 52.9 |
| 992 | 2012 | 13176 | 13196 | 51.4 | 47.6 | 4512 | 13946 | 13929 | 51.5 | 50 | 0.2 | 771 | 75.9 | 41.1 | 37 | 53.6 |
| 993 | 2013 | 3772 | 3792 | 51.2 | 42.9 | 4513 | 4444 | 4424 | 50.6 | 42.9 | 0.7 | 673 | 75.6 | 40.7 | 37 | 53.2 |
| 994 | 2014 | 13176 | 13196 | 51.4 | 47.6 | 4514 | 13320 | 13300 | 51.4 | 47.6 | 0 | 145 | 73.3 | 44.1 | 37 | 51.9 |
| 995 | 2015 | 8861 | 8880 | 50.2 | 45 | 4515 | 9245 | 9226 | 50 | 45 | 0.1 | 385 | 74.9 | 40.8 | 37 | 52.5 |
| 996 | 2016 | 8868 | 8889 | 50.4 | 40.9 | 4516 | 9310 | 9291 | 51.2 | 45 | 0.8 | 443 | 75.3 | 41.3 | 37 | 52.9 |
| 997 | 2017 | 16366 | 16384 | 50.3 | 52.6 | 4517 | 16774 | 16752 | 52.2 | 43.5 | 1.9 | 409 | 75.1 | 41.1 | 37 | 52.8 |
| 998 | 2018 | 9934 | 9953 | 50.7 | 50 | 4518 | 10183 | 10166 | 50.9 | 50 | 0.2 | 250 | 75.2 | 44 | 37 | 53 |
| 999 | 2019 | 9055 | 9079 | 52.8 | 40 | 4519 | 9342 | 9323 | 52.1 | 50 | 0.8 | 288 | 75.1 | 42.7 | 37 | 53.3 |
| 1000 | 2020 | 8868 | 8889 | 50.4 | 40.9 | 4520 | 9249 | 9231 | 50.8 | 47.4 | 0.4 | 382 | 75.1 | 41.4 | 37 | 52.8 |
| 1001 | 2021 | 16366 | 16385 | 52.9 | 55 | 4521 | 16774 | 16752 | 52.2 | 43.5 | 0.6 | 409 | 75.1 | 41.1 | 37 | 53.3 |
| 1002 | 2022 | 8868 | 8889 | 50.4 | 40.9 | 4522 | 9249 | 9230 | 51.5 | 45 | 1.1 | 382 | 75.1 | 41.4 | 37 | 52.8 |
| 1003 | 2023 | 9934 | 9953 | 50.7 | 50 | 4523 | 10183 | 10165 | 51.7 | 47.4 | 0.9 | 250 | 75.2 | 44 | 37 | 53 |
| 1004 | 2024 | 25772 | 25793 | 52.4 | 40.9 | 4524 | 26183 | 26163 | 51.7 | 42.9 | 0.7 | 412 | 74.8 | 40.3 | 37 | 53 |
| 1005 | 2025 | 13039 | 13057 | 51.1 | 52.6 | 4525 | 13155 | 13138 | 50.4 | 50 | 0.7 | 117 | 73.4 | 47 | 37 | 51.6 |
| 1006 | 2026 | 25771 | 25790 | 51.1 | 45 | 4526 | 26183 | 26164 | 51 | 45 | 0.1 | 413 | 74.8 | 40.4 | 37 | 52.8 |
| 1007 | 2027 | 25769 | 25786 | 50.3 | 50 | 4527 | 26183 | 26163 | 51.7 | 42.9 | 1.4 | 415 | 74.9 | 40.5 | 37 | 52.6 |
| 1008 | 2028 | 3794 | 3812 | 52.9 | 52.6 | 4528 | 4436 | 4417 | 52.2 | 50 | 0.6 | 643 | 75.4 | 40.4 | 37 | 53.6 |
| 1009 | 2029 | 887 | 905 | 50.1 | 47.4 | 4529 | 1480 | 1462 | 51.6 | 47.4 | 1.5 | 594 | 77.1 | 44.6 | 37 | 54.1 |
| 1010 | 2030 | 3794 | 3812 | 52.9 | 52.6 | 4530 | 4434 | 4416 | 51.5 | 52.6 | 1.4 | 641 | 75.4 | 40.4 | 37 | 53.3 |
| 1011 | 2031 | 12370 | 12388 | 50.1 | 47.4 | 4531 | 12994 | 12976 | 50.3 | 47.4 | 0.3 | 625 | 76.4 | 42.9 | 37 | 53.6 |
| 1012 | 2032 | 3797 | 3815 | 50.9 | 47.4 | 4532 | 4186 | 4168 | 51.8 | 52.6 | 0.9 | 390 | 75.3 | 41.8 | 37 | 53.1 |
| 1013 | 2033 | 3795 | 3813 | 52.1 | 52.6 | 4533 | 4435 | 4417 | 50.5 | 52.6 | 1.6 | 641 | 75.5 | 40.6 | 37 | 53.1 |
| 1014 | 2034 | 3795 | 3813 | 52.1 | 52.6 | 4534 | 4434 | 4416 | 51.5 | 52.6 | 0.6 | 640 | 75.4 | 40.5 | 37 | 53.3 |
| 1015 | 2035 | 13177 | 13197 | 50.3 | 42.9 | 4535 | 13323 | 13304 | 51.1 | 45 | 0.8 | 147 | 73.2 | 43.5 | 37 | 51.4 |
| 1016 | 2036 | 1046 | 1064 | 51.2 | 47.4 | 4536 | 1401 | 1382 | 50.6 | 45 | 0.6 | 356 | 75.6 | 43 | 37 | 53.2 |
| 1017 | 2037 | 16549 | 16567 | 54.9 | 52.6 | 4537 | 17057 | 17035 | 53 | 43.5 | 1.9 | 509 | 75.9 | 42.2 | 37 | 54.1 |
| 1018 | 2038 | 1046 | 1064 | 51.2 | 47.4 | 4538 | 1483 | 1464 | 51.3 | 45 | 0.1 | 438 | 76.2 | 43.6 | 37 | 53.8 |
| 1019 | 2039 | 16551 | 16568 | 51.1 | 50 | 4539 | 17056 | 17035 | 51.8 | 45.5 | 0.7 | 506 | 75.9 | 42.3 | 37 | 53.6 |
| 1020 | 2040 | 1046 | 1064 | 51.2 | 47.4 | 4540 | 1483 | 1465 | 50.5 | 47.4 | 0.6 | 438 | 76.2 | 43.6 | 37 | 53.6 |
| 1021 | 2041 | 1046 | 1064 | 51.2 | 47.4 | 4541 | 1484 | 1466 | 53.1 | 52.6 | 2 | 439 | 76.3 | 43.7 | 37 | 53.9 |
| 1022 | 2042 | 1046 | 1064 | 51.2 | 47.4 | 4542 | 1697 | 1676 | 51.7 | 40.9 | 0.5 | 652 | 76.9 | 43.9 | 37 | 54.2 |
| 1023 | 2043 | 1046 | 1064 | 51.2 | 47.4 | 4543 | 1697 | 1677 | 51 | 42.9 | 0.2 | 652 | 76.9 | 43.9 | 37 | 54.2 |
| 1024 | 2044 | 16555 | 16572 | 50.3 | 50 | 4544 | 17111 | 17090 | 51.1 | 40.9 | 0.8 | 557 | 76.1 | 42.5 | 37 | 53.5 |
| 1025 | 2045 | 1046 | 1064 | 51.2 | 47.4 | 4545 | 1697 | 1678 | 50.3 | 45 | 0.9 | 652 | 76.9 | 43.9 | 37 | 54 |
| 1026 | 2046 | 1046 | 1063 | 50.3 | 50 | 4546 | 1401 | 1382 | 50.6 | 45 | 0.2 | 356 | 75.6 | 43 | 37 | 53.1 |
| 1027 | 2047 | 3796 | 3814 | 50.8 | 52.6 | 4547 | 4435 | 4417 | 50.5 | 52.6 | 0.3 | 640 | 75.4 | 40.5 | 37 | 53.1 |
| 1028 | 2048 | 12232 | 12250 | 51.9 | 52.6 | 4548 | 12993 | 12975 | 51.4 | 47.4 | 0.5 | 762 | 76.5 | 42.5 | 37 | 54 |
| 1029 | 2049 | 12236 | 12256 | 51.2 | 42.9 | 4549 | 12739 | 12719 | 50.3 | 42.9 | 0.9 | 504 | 75.8 | 42.1 | 37 | 53.2 |
| 1030 | 2050 | 28937 | 28956 | 52.4 | 50 | 4550 | 29306 | 29288 | 53.5 | 52.6 | 1.1 | 370 | 76.6 | 45.1 | 37 | 54.4 |
| 1031 | 2051 | 12232 | 12250 | 51.9 | 52.6 | 4551 | 12996 | 12977 | 50.2 | 40 | 1.7 | 765 | 76.4 | 42.4 | 37 | 53.6 |
| 1032 | 2052 | 3234 | 3254 | 51.1 | 47.6 | 4552 | 3504 | 3485 | 50.4 | 45 | 0.7 | 271 | 74.4 | 41.3 | 37 | 52.3 |
| 1033 | 2053 | 3234 | 3254 | 51.1 | 47.6 | 4553 | 3503 | 3484 | 51.5 | 50 | 0.4 | 270 | 74.4 | 41.5 | 37 | 52.5 |
| 1034 | 2054 | 9922 | 9941 | 51.3 | 50 | 4554 | 10670 | 10649 | 51.3 | 40.9 | 0.1 | 749 | 75.8 | 40.9 | 37 | 53.5 |
| 1035 | 2055 | 3792 | 3810 | 52.9 | 52.6 | 4555 | 4434 | 4416 | 51.5 | 52.6 | 1.4 | 643 | 75.4 | 40.4 | 37 | 53.3 |
| 1036 | 2056 | 3234 | 3254 | 51.1 | 47.6 | 4556 | 3494 | 3473 | 50.4 | 40.9 | 0.6 | 261 | 74.1 | 41 | 37 | 52.1 |
| 1037 | 2057 | 4255 | 4276 | 51.7 | 45.5 | 4557 | 4608 | 4590 | 51.5 | 52.6 | 0.2 | 354 | 74.7 | 40.7 | 37 | 52.8 |
| 1038 | 2058 | 24562 | 24580 | 50.1 | 52.6 | 4558 | 24936 | 24919 | 51.8 | 50 | 1.7 | 375 | 75.6 | 42.7 | 37 | 53 |
| 1039 | 2059 | 24562 | 24580 | 50.1 | 52.6 | 4559 | 24938 | 24921 | 50.4 | 50 | 0.3 | 377 | 75.5 | 42.4 | 37 | 53 |
| 1040 | 2060 | 24562 | 24580 | 50.1 | 52.6 | 4560 | 25182 | 25164 | 51.4 | 47.4 | 1.3 | 621 | 75.9 | 41.7 | 37 | 53.3 |
| 1041 | 2061 | 24559 | 24579 | 52 | 52.4 | 4561 | 24936 | 24919 | 51.8 | 50 | 0.2 | 378 | 75.7 | 42.9 | 37 | 53.6 |
| 1042 | 2062 | 24559 | 24579 | 52 | 52.4 | 4562 | 24938 | 24921 | 50.4 | 50 | 1.6 | 380 | 75.6 | 42.6 | 37 | 53.1 |
| 1043 | 2063 | 1046 | 1063 | 50.3 | 50 | 4563 | 1697 | 1676 | 51.7 | 40.9 | 1.3 | 652 | 76.9 | 43.9 | 37 | 54 |
| 1044 | 2064 | 24482 | 24503 | 51.6 | 40.9 | 4564 | 24815 | 24792 | 53.4 | 41.7 | 1.8 | 334 | 75.4 | 42.8 | 37 | 53.4 |
| 1045 | 2065 | 13177 | 13197 | 50.3 | 42.9 | 4565 | 13326 | 13306 | 50.7 | 42.9 | 0.4 | 150 | 73.2 | 43.3 | 37 | 51.4 |
| 1046 | 2066 | 24480 | 24502 | 54.2 | 47.8 | 4566 | 24815 | 24791 | 54.5 | 40 | 0.3 | 336 | 75.6 | 43.2 | 37 | 54.3 |
| 1047 | 2067 | 17840 | 17859 | 50.8 | 45 | 4567 | 18223 | 18206 | 51.8 | 50 | 1 | 384 | 74.7 | 40.4 | 37 | 52.6 |
| 1048 | 2068 | 24480 | 24500 | 53.2 | 47.6 | 4568 | 24815 | 24792 | 53.4 | 41.7 | 0.2 | 336 | 75.6 | 43.2 | 37 | 54 |
| 1049 | 2069 | 17840 | 17859 | 50.8 | 45 | 4569 | 18231 | 18210 | 52.2 | 45.5 | 1.4 | 392 | 74.8 | 40.6 | 37 | 52.7 |
| 1050 | 2070 | 28821 | 28840 | 51.8 | 45 | 4570 | 29298 | 29279 | 52.6 | 55 | 0.8 | 478 | 77 | 45.2 | 37 | 54.6 |
| 1051 | 2071 | 24418 | 24440 | 55 | 47.8 | 4571 | 24517 | 24494 | 53.2 | 41.7 | 1.8 | 100 | 70.8 | 43 | 37 | 50.6 |
| 1052 | 2072 | 13701 | 13722 | 50.4 | 40.9 | 4572 | 14058 | 14040 | 51.4 | 52.6 | 1 | 358 | 74.5 | 40.2 | 37 | 52.4 |
| 1053 | 2073 | 28821 | 28840 | 51.8 | 45 | 4573 | 29358 | 29339 | 52.8 | 50 | 1 | 538 | 77.1 | 45 | 37 | 54.6 |
| 1054 | 2074 | 17792 | 17813 | 51.6 | 40.9 | 4574 | 18233 | 18214 | 52 | 50 | 0.4 | 442 | 75.1 | 40.7 | 37 | 53.1 |
| 1055 | 2075 | 24420 | 24440 | 50.8 | 42.9 | 4575 | 25079 | 25061 | 52.7 | 52.6 | 1.9 | 660 | 75.7 | 41.1 | 37 | 53.3 |
| 1056 | 2076 | 28821 | 28839 | 51.1 | 47.4 | 4576 | 29298 | 29279 | 52.6 | 55 | 1.5 | 478 | 77 | 45.2 | 37 | 54.3 |
| 1057 | 2077 | 3796 | 3814 | 50.8 | 52.6 | 4577 | 4434 | 4416 | 51.5 | 52.6 | 0.7 | 639 | 75.4 | 40.4 | 37 | 53.1 |
| 1058 | 2078 | 28821 | 28839 | 51.1 | 47.4 | 4578 | 29358 | 29339 | 52.8 | 50 | 1.7 | 538 | 77.1 | 45 | 37 | 54.4 |
| 1059 | 2079 | 28820 | 28838 | 53.7 | 52.6 | 4579 | 29298 | 29279 | 52.6 | 55 | 1.1 | 479 | 77.1 | 45.3 | 37 | 54.8 |
| 1060 | 2080 | 24418 | 24439 | 52.9 | 45.5 | 4580 | 25079 | 25061 | 52.7 | 52.6 | 0.2 | 662 | 75.8 | 41.2 | 37 | 54 |
| 1061 | 2081 | 28820 | 28838 | 53.7 | 52.6 | 4581 | 29358 | 29339 | 52.8 | 50 | 0.9 | 539 | 77.1 | 45.1 | 37 | 54.9 |
| 1062 | 2082 | 27369 | 27389 | 52.5 | 47.6 | 4582 | 27468 | 27451 | 51.1 | 50 | 1.4 | 100 | 71.2 | 44 | 38 | 50.3 |
| 1063 | 2083 | 7725 | 7742 | 50 | 50 | 4583 | 8187 | 8167 | 50.4 | 42.9 | 0.3 | 463 | 75.6 | 41.9 | 38 | 53 |
| 1064 | 2084 | 16549 | 16567 | 54.9 | 52.6 | 4584 | 17040 | 17021 | 53.4 | 50 | 1.5 | 492 | 75.9 | 42.3 | 38 | 54.2 |
| 1065 | 2085 | 3221 | 3239 | 51.5 | 52.6 | 4585 | 3500 | 3481 | 51.2 | 50 | 0.3 | 280 | 74.6 | 41.8 | 38 | 52.7 |
| 1066 | 2086 | 16549 | 16567 | 54.9 | 52.6 | 4586 | 17041 | 17022 | 54.1 | 50 | 0.8 | 493 | 75.8 | 42.2 | 38 | 54.4 |
| 1067 | 2087 | 20138 | 20158 | 50.1 | 42.9 | 4587 | 20615 | 20597 | 50.6 | 47.4 | 0.5 | 478 | 75 | 40.4 | 38 | 52.7 |
| 1068 | 2088 | 20078 | 20099 | 50.5 | 40.9 | 4588 | 20615 | 20597 | 50.6 | 47.4 | 0.1 | 538 | 75.3 | 40.5 | 38 | 52.9 |
| 1069 | 2089 | 13039 | 13057 | 51.1 | 52.6 | 4589 | 13325 | 13305 | 50.5 | 47.6 | 0.6 | 287 | 75.8 | 44.6 | 38 | 53.3 |
| 1070 | 2090 | 16549 | 16567 | 54.9 | 52.6 | 4590 | 17041 | 17023 | 53.5 | 52.6 | 1.4 | 493 | 75.8 | 42.2 | 38 | 54.2 |
| 1071 | 2091 | 13701 | 13725 | 53.6 | 40 | 4591 | 14124 | 14106 | 52.4 | 52.6 | 1.2 | 424 | 75.1 | 41 | 38 | 53.4 |
| 1072 | 2092 | 12975 | 12993 | 51.4 | 47.4 | 4592 | 13320 | 13300 | 51.4 | 47.6 | 0 | 346 | 76.1 | 44.2 | 38 | 53.8 |
| 1073 | 2093 | 16548 | 16566 | 54.9 | 52.6 | 4593 | 16779 | 16758 | 53.5 | 50 | 1.4 | 232 | 74 | 41.4 | 38 | 52.9 |
| 1074 | 2094 | 3361 | 3381 | 50.5 | 42.9 | 4594 | 3500 | 3481 | 51.2 | 50 | 0.7 | 140 | 74.1 | 46.4 | 38 | 52.1 |
| 1075 | 2095 | 3361 | 3381 | 50.5 | 42.9 | 4595 | 3503 | 3484 | 51.5 | 50 | 1 | 143 | 74.4 | 46.9 | 38 | 52.3 |
| 1076 | 2096 | 16368 | 16387 | 50.2 | 45 | 4596 | 16780 | 16760 | 51.4 | 42.9 | 1.2 | 413 | 74.9 | 40.7 | 38 | 52.6 |
| 1077 | 2097 | 7725 | 7742 | 50 | 50 | 4597 | 8188 | 8168 | 50.4 | 42.9 | 0.3 | 464 | 75.6 | 41.8 | 38 | 53 |
| 1078 | 2098 | 8867 | 8887 | 52.3 | 47.6 | 4598 | 9597 | 9573 | 53.4 | 40 | 1.1 | 731 | 75.9 | 41.2 | 38 | 53.9 |
| 1079 | 2099 | 2223 | 2244 | 51.4 | 45.5 | 4599 | 2672 | 2654 | 50.9 | 52.6 | 0.5 | 450 | 77 | 45.3 | 38 | 54.3 |
| 1080 | 2100 | 10242 | 10265 | 51.2 | 41.7 | 4600 | 10605 | 10588 | 51.1 | 50 | 0.2 | 364 | 74.5 | 40.1 | 38 | 52.6 |
| 1081 | 2101 | 8867 | 8888 | 52.7 | 45.5 | 4601 | 9253 | 9235 | 51.6 | 47.4 | 1.1 | 387 | 75.1 | 41.3 | 38 | 53.2 |
| 1082 | 2102 | 3361 | 3381 | 50.5 | 42.9 | 4602 | 3504 | 3485 | 50.4 | 45 | 0.1 | 144 | 74.3 | 46.5 | 38 | 52.2 |
| 1083 | 2103 | 98 | 118 | 50.6 | 42.9 | 4603 | 314 | 296 | 50.6 | 47.4 | 0 | 217 | 75.9 | 46.5 | 38 | 53.4 |
| 1084 | 2104 | 12233 | 12251 | 51.1 | 52.6 | 4604 | 12498 | 12480 | 50 | 47.4 | 1.1 | 266 | 74.8 | 42.5 | 38 | 52.5 |
| 1085 | 2105 | 9926 | 9944 | 50.5 | 52.6 | 4605 | 10455 | 10434 | 51.1 | 40.9 | 0.6 | 530 | 75.3 | 40.6 | 38 | 52.9 |
| 1086 | 2106 | 3360 | 3380 | 51.4 | 42.9 | 4606 | 3497 | 3478 | 51.3 | 50 | 0.1 | 138 | 74 | 46.4 | 38 | 52.3 |
| 1087 | 2107 | 9926 | 9944 | 50.5 | 52.6 | 4607 | 10455 | 10435 | 50.5 | 42.9 | 0 | 530 | 75.3 | 40.6 | 38 | 52.9 |
| 1088 | 2108 | 10140 | 10159 | 52.4 | 50 | 4608 | 10608 | 10589 | 51 | 50 | 1.4 | 469 | 75 | 40.3 | 38 | 52.9 |
| 1089 | 2109 | 9931 | 9950 | 50.2 | 45 | 4609 | 10455 | 10435 | 50.5 | 42.9 | 0.3 | 525 | 75.3 | 40.6 | 38 | 52.8 |
| 1090 | 2110 | 3219 | 3238 | 50.7 | 50 | 4610 | 3500 | 3481 | 51.2 | 50 | 0.5 | 282 | 74.7 | 41.8 | 38 | 52.6 |
| 1091 | 2111 | 3219 | 3238 | 50.7 | 50 | 4611 | 3497 | 3478 | 51.3 | 50 | 0.6 | 279 | 74.7 | 41.9 | 38 | 52.6 |
| 1092 | 2112 | 3360 | 3380 | 51.4 | 42.9 | 4612 | 3500 | 3481 | 51.2 | 50 | 0.3 | 141 | 74 | 46.1 | 38 | 52.3 |
| 1093 | 2113 | 3360 | 3380 | 51.4 | 42.9 | 4613 | 3503 | 3484 | 51.5 | 50 | 0 | 144 | 74.3 | 46.5 | 38 | 52.5 |
| 1094 | 2114 | 2223 | 2244 | 51.4 | 45.5 | 4614 | 2672 | 2653 | 51.6 | 50 | 0.2 | 450 | 77 | 45.3 | 38 | 54.4 |
| 1095 | 2115 | 9922 | 9941 | 51.3 | 50 | 4615 | 10449 | 10428 | 51.9 | 40.9 | 0.7 | 528 | 75.4 | 40.9 | 38 | 53.3 |
| 1096 | 2116 | 13039 | 13057 | 51.1 | 52.6 | 4616 | 13312 | 13294 | 51 | 52.6 | 0.1 | 274 | 75.7 | 44.5 | 38 | 53.4 |
| 1097 | 2117 | 15951 | 15973 | 52.1 | 43.5 | 4617 | 16174 | 16154 | 50.4 | 42.9 | 1.7 | 224 | 73.5 | 40.6 | 38 | 51.7 |
| 1098 | 2118 | 13176 | 13196 | 51.4 | 47.6 | 4618 | 13545 | 13526 | 52.9 | 55 | 1.5 | 370 | 77 | 46.2 | 38 | 54.4 |
| 1099 | 2119 | 11541 | 11562 | 51.5 | 40.9 | 4619 | 11983 | 11965 | 53 | 52.6 | 1.5 | 443 | 75 | 40.6 | 38 | 53.1 |
| 1100 | 2120 | 2429 | 2447 | 50.2 | 47.4 | 4620 | 3056 | 3038 | 50.8 | 52.6 | 0.6 | 628 | 76.3 | 42.7 | 38 | 53.6 |
| 1101 | 2121 | 11545 | 11563 | 50.8 | 47.4 | 4621 | 12258 | 12238 | 50.3 | 42.9 | 0.5 | 714 | 76.2 | 42 | 38 | 53.5 |
| 1102 | 2122 | 8868 | 8889 | 50.4 | 40.9 | 4622 | 9245 | 9226 | 50 | 45 | 0.4 | 378 | 74.9 | 41 | 38 | 52.6 |
| 1103 | 2123 | 27361 | 27380 | 52.4 | 55 | 4623 | 27466 | 27448 | 52.3 | 52.6 | 0.1 | 106 | 72.5 | 46.2 | 38 | 51.5 |
| 1104 | 2124 | 8861 | 8880 | 50.2 | 45 | 4624 | 9340 | 9319 | 50.8 | 45.5 | 0.6 | 480 | 75.5 | 41.5 | 38 | 53 |
| 1105 | 2125 | 1784 | 1802 | 51.8 | 52.6 | 4625 | 2113 | 2094 | 50.1 | 45 | 1.7 | 330 | 76 | 44.2 | 38 | 53.3 |
| 1106 | 2126 | 8868 | 8889 | 50.4 | 40.9 | 4626 | 9107 | 9086 | 51.6 | 45.5 | 1.2 | 240 | 74 | 41.2 | 38 | 52 |
| 1107 | 2127 | 19795 | 19814 | 50.4 | 45 | 4627 | 20099 | 20078 | 50.5 | 40.9 | 0 | 305 | 74.4 | 40.7 | 38 | 52.3 |
| 1108 | 2128 | 26708 | 26731 | 54.2 | 41.7 | 4628 | 27347 | 27324 | 52.3 | 41.7 | 1.9 | 640 | 75.6 | 40.8 | 38 | 53.7 |
| 1109 | 2129 | 19794 | 19813 | 50 | 50 | 4629 | 19920 | 19899 | 50.2 | 40.9 | 0.2 | 127 | 72.3 | 43.3 | 38 | 50.7 |
| 1110 | 2130 | 3031 | 3051 | 51.3 | 52.4 | 4630 | 3650 | 3631 | 53.1 | 50 | 1.8 | 620 | 76.5 | 43.1 | 38 | 54 |
| 1111 | 2131 | 3031 | 3051 | 51.3 | 52.4 | 4631 | 3647 | 3628 | 50.6 | 45 | 0.7 | 617 | 76.4 | 42.9 | 38 | 53.8 |
| 1112 | 2132 | 19794 | 19813 | 50 | 50 | 4632 | 19922 | 19902 | 50 | 42.9 | 0 | 129 | 72.5 | 43.4 | 38 | 50.8 |
| 1113 | 2133 | 12236 | 12256 | 51.2 | 42.9 | 4633 | 12994 | 12976 | 50.3 | 47.4 | 0.8 | 759 | 76.4 | 42.4 | 38 | 53.7 |
| 1114 | 2134 | 26708 | 26731 | 54.2 | 41.7 | 4634 | 27467 | 27449 | 52.8 | 47.4 | 1.4 | 760 | 76 | 41.3 | 38 | 54.1 |
| 1115 | 2135 | 19716 | 19737 | 52.2 | 45.5 | 4635 | 19922 | 19901 | 51.5 | 45.5 | 0.7 | 207 | 73.5 | 41.1 | 38 | 52 |
| 1116 | 2136 | 19715 | 19735 | 52.5 | 47.6 | 4636 | 19922 | 19901 | 51.5 | 45.5 | 0.9 | 208 | 73.6 | 41.3 | 38 | 52.1 |
| 1117 | 2137 | 3360 | 3379 | 50.7 | 45 | 4637 | 3503 | 3484 | 51.5 | 50 | 0.7 | 144 | 74.3 | 46.5 | 38 | 52.3 |
| 1118 | 2138 | 9055 | 19079 | 52.8 | 40 | 4638 | 9364 | 9346 | 53.9 | 52.6 | 1.1 | 310 | 75.3 | 42.9 | 38 | 53.7 |
| 1119 | 2139 | 1782 | 1801 | 52.7 | 50 | 4639 | 1881 | 1861 | 54.5 | 52.4 | 1.8 | 100 | 72.4 | 47 | 38 | 51.6 |
| 1120 | 2140 | 26708 | 26727 | 50 | 45 | 4640 | 27468 | 27450 | 51.9 | 47.4 | 1.8 | 761 | 75.9 | 41.3 | 38 | 53.3 |
| 1121 | 2141 | 26708 | 26727 | 50 | 45 | 4641 | 27468 | 27451 | 51.1 | 50 | 1.1 | 761 | 75.9 | 41.3 | 38 | 53.3 |
| 1122 | 2142 | 4593 | 4613 | 51.5 | 47.6 | 4642 | 4995 | 4975 | 51.7 | 42.9 | 0.1 | 403 | 76 | 43.4 | 38 | 53.8 |
| 1123 | 2143 | 19709 | 19730 | 51.3 | 40.9 | 4643 | 19930 | 19911 | 50.7 | 50 | 0.5 | 222 | 74 | 41.9 | 38 | 52.1 |
| 1124 | 2144 | 26421 | 26441 | 51.5 | 42.9 | 4644 | 26587 | 26570 | 50.2 | 50 | 1.3 | 167 | 72.3 | 40.1 | 38 | 50.8 |
| 1125 | 2145 | 18979 | 19000 | 51.6 | 45.5 | 4645 | 19217 | 19195 | 51.7 | 43.5 | 0 | 239 | 73.5 | 40.2 | 38 | 52.1 |
| 1126 | 2146 | 18703 | 18724 | 53.5 | 50 | 4646 | 19476 | 19453 | 53.5 | 41.7 | 0 | 774 | 75.6 | 40.3 | 38 | 54 |
| 1127 | 2147 | 4255 | 4276 | 51.7 | 45.5 | 4647 | 4708 | 4690 | 50.3 | 47.4 | 1.4 | 454 | 75.1 | 40.7 | 38 | 52.8 |
| 1128 | 2148 | 3232 | 3252 | 51.1 | 47.6 | 4648 | 3503 | 3484 | 51.5 | 50 | 0.4 | 272 | 74.6 | 41.9 | 38 | 52.6 |
| 1129 | 2149 | 26421 | 26441 | 51.5 | 42.9 | 4649 | 26656 | 26636 | 51.3 | 47.6 | 0.2 | 236 | 74.2 | 41.9 | 38 | 52.5 |
| 1130 | 2150 | 3232 | 3252 | 51.1 | 47.6 | 4650 | 3504 | 3485 | 50.4 | 45 | 0.7 | 273 | 74.6 | 41.8 | 38 | 52.4 |
| 1131 | 2151 | 26421 | 26441 | 51.5 | 42.9 | 4651 | 26660 | 26641 | 50.2 | 50 | 1.3 | 240 | 74.2 | 41.7 | 38 | 52.1 |
| 1132 | 2152 | 26421 | 26441 | 51.5 | 42.9 | 4652 | 26683 | 26665 | 52.7 | 52.6 | 1.2 | 263 | 74.8 | 42.6 | 38 | 52.9 |
| 1133 | 2153 | 26421 | 26441 | 51.5 | 42.9 | 4653 | 26686 | 26669 | 50.5 | 50 | 0.9 | 266 | 74.8 | 42.5 | 38 | 52.6 |
| 1134 | 2154 | 26421 | 26441 | 51.5 | 42.9 | 4654 | 26691 | 26673 | 51.3 | 47.4 | 0.1 | 271 | 74.8 | 42.4 | 38 | 52.9 |
| 1135 | 2155 | 18704 | 18724 | 50.8 | 47.6 | 4655 | 19476 | 19456 | 50.5 | 42.9 | 0.3 | 773 | 75.5 | 40.2 | 38 | 53.1 |
| 1136 | 2156 | 18704 | 18724 | 50.8 | 47.6 | 4656 | 19482 | 19463 | 50.1 | 45 | 0.7 | 779 | 75.5 | 40.2 | 38 | 53 |
| 1137 | 2157 | 942 | 960 | 52.1 | 52.6 | 4657 | 1498 | 1481 | 51 | 50 | 1.1 | 557 | 76.9 | 44.5 | 38 | 54.3 |
| 1138 | 2158 | 942 | 960 | 52.1 | 52.6 | 4658 | 1497 | 1480 | 50.3 | 50 | 1.9 | 556 | 77 | 44.6 | 38 | 54.1 |
| 1139 | 2159 | 13040 | 13059 | 50.9 | 50 | 4659 | 13312 | 13294 | 51 | 52.6 | 0.1 | 273 | 75.6 | 44.3 | 38 | 53.3 |
| 1140 | 2160 | 18696 | 18715 | 51.7 | 50 | 4660 | 19476 | 19453 | 53.5 | 41.7 | 1.8 | 781 | 75.6 | 40.3 | 38 | 53.5 |
| 1141 | 2161 | 18696 | 18715 | 51.7 | 50 | 4661 | 19476 | 19456 | 50.5 | 42.9 | 1.3 | 781 | 75.6 | 40.3 | 38 | 53.1 |
| 1142 | 2162 | 3232 | 3251 | 50.3 | 50 | 4662 | 3503 | 3484 | 51.5 | 50 | 1.1 | 272 | 74.6 | 41.9 | 38 | 52.4 |
| 1143 | 2163 | 3031 | 3051 | 51.3 | 52.4 | 4663 | 3646 | 3625 | 52 | 40.9 | 0.7 | 616 | 76.4 | 42.9 | 38 | 54 |
| 1144 | 2164 | 9130 | 9150 | 51.3 | 42.9 | 4664 | 9560 | 9541 | 50.9 | 45 | 0.4 | 431 | 75.3 | 41.3 | 38 | 53 |
| 1145 | 2165 | 18224 | 18243 | 53.1 | 50 | 4665 | 18696 | 18672 | 53.9 | 40 | 0.8 | 473 | 75.7 | 42.1 | 38 | 54 |
| 1146 | 2166 | 18224 | 18243 | 53.1 | 50 | 4666 | 18696 | 18673 | 53.4 | 41.7 | 0.3 | 473 | 75.7 | 42.1 | 38 | 54 |
| 1147 | 2167 | 18225 | 18243 | 51.4 | 52.6 | 4667 | 18697 | 18679 | 51.9 | 52.6 | 0.5 | 473 | 75.8 | 42.3 | 38 | 53.6 |
| 1148 | 2168 | 9130 | 9150 | 51.3 | 42.9 | 4668 | 9560 | 9540 | 51.6 | 42.9 | 0.3 | 431 | 75.3 | 41.3 | 38 | 53.2 |
| 1149 | 2169 | 8866 | 8885 | 51.1 | 45 | 4669 | 9252 | 9235 | 50.1 | 50 | 1 | 387 | 75.1 | 41.3 | 38 | 52.7 |
| 1150 | 2170 | 9130 | 9150 | 51.3 | 42.9 | 4670 | 9559 | 9539 | 50.6 | 42.9 | 0.7 | 430 | 75.3 | 41.4 | 38 | 53 |
| 1151 | 2171 | 12267 | 12290 | 54.5 | 41.7 | 4671 | 12501 | 12480 | 53.5 | 45.5 | 1 | 235 | 74.3 | 42.1 | 38 | 53.2 |
| 1152 | 2172 | 3427 | 3446 | 52.7 | 50 | 4672 | 3650 | 3631 | 53.1 | 50 | 0.4 | 224 | 74.3 | 42.4 | 38 | 52.9 |
| 1153 | 2173 | 3427 | 3446 | 52.7 | 50 | 4673 | 3648 | 3628 | 52.3 | 42.9 | 0.4 | 222 | 74 | 41.9 | 38 | 52.6 |
| 1154 | 2174 | 26039 | 26058 | 54 | 55 | 4674 | 26184 | 26164 | 52.4 | 42.9 | 1.6 | 146 | 71.8 | 40.4 | 38 | 51.1 |
| 1155 | 2175 | 3230 | 3249 | 50.1 | 45 | 4675 | 3503 | 3484 | 51.5 | 50 | 1.4 | 274 | 74.5 | 41.6 | 38 | 52.3 |
| 1156 | 2176 | 3230 | 3249 | 50.1 | 45 | 4676 | 3504 | 3485 | 50.4 | 45 | 0.3 | 275 | 74.4 | 41.5 | 38 | 52.2 |
| 1157 | 2177 | 3427 | 3446 | 52.7 | 50 | 4677 | 3646 | 3625 | 52 | 40.9 | 0.6 | 220 | 74 | 41.8 | 38 | 52.5 |
| 1158 | 2178 | 3429 | 3449 | 50.4 | 42.9 | 4678 | 3647 | 3628 | 50.6 | 45 | 0.2 | 219 | 73.9 | 41.6 | 38 | 51.9 |
| 1159 | 2179 | 3429 | 3449 | 50.4 | 42.9 | 4679 | 3646 | 3625 | 52 | 40.9 | 1.6 | 218 | 73.7 | 41.3 | 38 | 51.8 |
| 1160 | 2180 | 8866 | 8885 | 51.1 | 45 | 4680 | 9249 | 9231 | 50.8 | 47.4 | 0.3 | 384 | 75.1 | 41.4 | 38 | 52.9 |
| 1161 | 2181 | 3428 | 3449 | 52.8 | 45.5 | 4681 | 3650 | 3631 | 53.1 | 50 | 0.3 | 223 | 74.2 | 42.2 | 38 | 52.8 |
| 1162 | 2182 | 18077 | 18098 | 52.9 | 50 | 4682 | 18696 | 18672 | 53.9 | 40 | 1 | 620 | 76.2 | 42.4 | 38 | 54.3 |
| 1163 | 2183 | 18078 | 18098 | 51.5 | 47.6 | 4683 | 18696 | 18673 | 53.4 | 41.7 | 1.9 | 619 | 76.2 | 42.3 | 38 | 53.9 |
| 1164 | 2184 | 8866 | 8885 | 51.1 | 45 | 4684 | 9249 | 9230 | 51.5 | 45 | 0.4 | 384 | 75.1 | 41.4 | 38 | 53 |
| 1165 | 2185 | 3229 | 3248 | 50.6 | 50 | 4685 | 3503 | 3484 | 51.5 | 50 | 0.8 | 275 | 74.6 | 41.8 | 38 | 52.5 |
| 1166 | 2186 | 12267 | 12290 | 54.5 | 41.7 | 4686 | 12495 | 12476 | 52.7 | 45 | 1.9 | 229 | 74.1 | 41.9 | 38 | 52.8 |
| 1167 | 2187 | 8220 | 8240 | 54 | 47.6 | 4687 | 8929 | 8910 | 54.5 | 55 | 0.4 | 710 | 75.4 | 40 | 38 | 54.1 |
| 1168 | 2188 | 18080 | 18098 | 51.2 | 52.6 | 4688 | 18238 | 18219 | 50.3 | 45 | 0.9 | 159 | 74 | 44.7 | 38 | 52 |
| 1169 | 2189 | 18080 | 18098 | 51.2 | 52.6 | 4689 | 18239 | 18220 | 50 | 45 | 1.2 | 160 | 73.9 | 44.4 | 38 | 51.8 |
| 1170 | 2190 | 18080 | 18098 | 51.2 | 52.6 | 4690 | 18697 | 18679 | 51.9 | 52.6 | 0.7 | 618 | 76.3 | 42.6 | 38 | 53.8 |
| 1171 | 2191 | 18076 | 18097 | 53.1 | 45.5 | 4691 | 18712 | 18693 | 54.8 | 55 | 1.7 | 637 | 76.3 | 42.5 | 38 | 54.4 |
| 1172 | 2192 | 8866 | 8885 | 51.1 | 45 | 4692 | 9245 | 9226 | 50 | 45 | 1.1 | 380 | 75 | 41.1 | 38 | 52.6 |
| 1173 | 2193 | 943 | 961 | 50.3 | 47.4 | 4693 | 1498 | 1481 | 51 | 50 | 0.8 | 556 | 76.9 | 44.4 | 38 | 54 |
| 1174 | 2194 | 18075 | 18095 | 50.6 | 47.6 | 4694 | 18642 | 18622 | 50.5 | 42.9 | 0.1 | 568 | 76.2 | 42.6 | 38 | 53.6 |
| 1175 | 2195 | 18075 | 18095 | 50.6 | 47.6 | 4695 | 18662 | 18641 | 50.4 | 40.9 | 0.2 | 588 | 76.3 | 42.7 | 38 | 53.6 |
| 1176 | 2196 | 8866 | 8885 | 51.1 | 45 | 4696 | 9107 | 9086 | 51.6 | 45.5 | 0.5 | 242 | 74.1 | 41.3 | 38 | 52.3 |
| 1177 | 2197 | 943 | 961 | 50.3 | 47.4 | 4697 | 1497 | 1480 | 50.3 | 50 | 0 | 555 | 76.9 | 44.5 | 38 | 54 |
| 1178 | 2198 | 7400 | 7417 | 50.2 | 50 | 4698 | 8190 | 8172 | 50.3 | 47.4 | 0.1 | 791 | 76.4 | 42.2 | 38 | 53.6 |
| 1179 | 2199 | 13039 | 13058 | 51.8 | 50 | 4699 | 13314 | 13297 | 51 | 50 | 0.9 | 276 | 75.7 | 44.6 | 38 | 53.4 |
| 1180 | 2200 | 7725 | 7743 | 50.8 | 47.4 | 4700 | 8187 | 8167 | 50.4 | 42.9 | 0.5 | 463 | 75.6 | 41.9 | 38 | 53.1 |
| 1181 | 2201 | 18074 | 18094 | 51.1 | 42.9 | 4701 | 18642 | 18622 | 50.5 | 42.9 | 0.5 | 569 | 76.2 | 42.5 | 38 | 53.6 |
| 1182 | 2202 | 25782 | 25805 | 52.1 | 41.7 | 4702 | 26174 | 26153 | 51 | 40.9 | 1.1 | 393 | 74.8 | 40.5 | 38 | 52.8 |
| 1183 | 2203 | 9131 | 9151 | 50.4 | 42.9 | 4703 | 9560 | 9541 | 50.9 | 45 | 0.5 | 430 | 75.3 | 41.4 | 38 | 52.9 |
| 1184 | 2204 | 25782 | 25805 | 52.1 | 41.7 | 4704 | 26183 | 26162 | 52.8 | 45.5 | 0.7 | 402 | 74.7 | 40.3 | 38 | 53.1 |
| 1185 | 2205 | 9131 | 9151 | 50.4 | 42.9 | 4705 | 9560 | 9540 | 51.6 | 42.9 | 1.2 | 430 | 75.3 | 41.4 | 38 | 52.9 |
| 1186 | 2206 | 7725 | 7743 | 50.8 | 47.4 | 4706 | 8188 | 8168 | 50.4 | 42.9 | 0.5 | 464 | 75.6 | 41.8 | 38 | 53.1 |
| 1187 | 2207 | 985 | 1004 | 51.1 | 50 | 4707 | 1494 | 1476 | 50.7 | 47.4 | 0.4 | 510 | 76.5 | 43.7 | 38 | 53.9 |
| 1188 | 2208 | 13039 | 13058 | 51.8 | 50 | 4708 | 13323 | 13304 | 51.1 | 45 | 0.7 | 285 | 75.8 | 44.6 | 38 | 53.5 |
| 1189 | 2209 | 9131 | 9151 | 50.4 | 42.9 | 4709 | 9559 | 9539 | 50.6 | 42.9 | 0.3 | 429 | 75.3 | 41.5 | 38 | 52.9 |
| 1190 | 2210 | 12352 | 12375 | 52.9 | 41.7 | 4710 | 12499 | 12480 | 51.8 | 45 | 1.1 | 148 | 73.3 | 43.9 | 38 | 52 |
| 1191 | 2211 | 3225 | 3244 | 52.4 | 55 | 4711 | 3646 | 3625 | 52 | 40.9 | 0.4 | 422 | 75.4 | 41.7 | 38 | 53.5 |
| 1192 | 2212 | 25676 | 25697 | 51.9 | 40.9 | 4712 | 25784 | 25765 | 53.3 | 50 | 1.4 | 109 | 70.3 | 40.4 | 38 | 49.9 |
| 1193 | 2213 | 25363 | 25381 | 51.1 | 52.6 | 4713 | 25548 | 25531 | 51.1 | 50 | 0 | 186 | 73.7 | 42.5 | 38 | 52 |
| 1194 | 2214 | 25363 | 25381 | 51.1 | 52.6 | 4714 | 25645 | 25626 | 50.8 | 45 | 0.4 | 283 | 74.2 | 40.6 | 38 | 52.3 |
| 1195 | 2215 | 18074 | 18093 | 50.3 | 45 | 4715 | 18642 | 18622 | 50.5 | 42.9 | 0.2 | 569 | 76.2 | 42.5 | 38 | 53.5 |
| 1196 | 2216 | 3225 | 3244 | 52.4 | 55 | 4716 | 3647 | 3628 | 50.6 | 45 | 1.8 | 423 | 75.5 | 41.8 | 38 | 53.1 |
| 1197 | 2217 | 3225 | 3244 | 52.4 | 55 | 4717 | 3650 | 3631 | 53.1 | 50 | 0.7 | 426 | 75.5 | 42 | 38 | 53.7 |
| 1198 | 2218 | 12352 | 12375 | 52.9 | 41.7 | 4718 | 12494 | 12476 | 52.2 | 47.4 | 0.6 | 143 | 73.2 | 44.1 | 38 | 52 |
| 1199 | 2219 | 13039 | 13058 | 51.8 | 50 | 4719 | 13326 | 13306 | 50.7 | 42.9 | 1.2 | 288 | 75.8 | 44.4 | 38 | 53.4 |
| 1200 | 2220 | 7617 | 7636 | 50.9 | 50 | 4720 | 8188 | 8169 | 50.5 | 45 | 0.5 | 572 | 76.1 | 42.3 | 38 | 53.5 |
| 1201 | 2221 | 988 | 1006 | 52.2 | 52.6 | 4721 | 1697 | 1678 | 50.3 | 45 | 2 | 710 | 76.9 | 43.8 | 38 | 54 |
| 1202 | 2222 | 12232 | 12250 | 51.9 | 52.6 | 4722 | 12739 | 12719 | 50.3 | 42.9 | 1.7 | 508 | 75.8 | 42.1 | 38 | 53.3 |
| 1203 | 2223 | 12232 | 12250 | 51.9 | 52.6 | 4723 | 12739 | 12718 | 51 | 40.9 | 1 | 508 | 75.8 | 42.1 | 38 | 53.5 |
| 1204 | 2224 | 988 | 1006 | 52.2 | 52.6 | 4724 | 1697 | 1677 | 51 | 42.9 | 1.2 | 710 | 76.9 | 43.8 | 38 | 54.2 |
| 1205 | 2225 | 8867 | 8886 | 50.7 | 50 | 4725 | 9341 | 9322 | 51.1 | 50 | 0.5 | 475 | 75.7 | 41.9 | 38 | 53.3 |
| 1206 | 2226 | 3223 | 3242 | 51.8 | 55 | 4726 | 3650 | 3631 | 53.1 | 50 | 1.3 | 428 | 75.6 | 42.1 | 38 | 53.6 |
| 1207 | 2227 | 8867 | 8886 | 50.7 | 50 | 4727 | 9340 | 9319 | 50.8 | 45.5 | 0.1 | 474 | 75.6 | 41.8 | 38 | 53.2 |
| 1208 | 2228 | 988 | 1006 | 52.2 | 52.6 | 4728 | 1697 | 1676 | 51.7 | 40.9 | 0.6 | 710 | 76.9 | 43.8 | 38 | 54.4 |
| 1209 | 2229 | 988 | 1006 | 52.2 | 52.6 | 4729 | 1694 | 1673 | 51.7 | 40.9 | 0.5 | 707 | 76.9 | 43.8 | 38 | 54.5 |
| 1210 | 2230 | 988 | 1006 | 52.2 | 52.6 | 4730 | 1494 | 1476 | 50.7 | 47.4 | 1.5 | 507 | 76.5 | 43.8 | 38 | 53.9 |
| 1211 | 2231 | 9931 | 9950 | 50.2 | 45 | 4731 | 10455 | 10434 | 51.1 | 40.9 | 1 | 525 | 75.3 | 40.6 | 38 | 52.8 |
| 1212 | 2232 | 3224 | 3242 | 50.5 | 52.6 | 4732 | 3646 | 3625 | 52 | 40.9 | 1.5 | 423 | 75.4 | 41.6 | 38 | 53 |
| 1213 | 2233 | 3224 | 3242 | 50.5 | 52.6 | 4733 | 3647 | 3628 | 50.6 | 45 | 0.1 | 424 | 75.4 | 41.7 | 38 | 53.1 |
| 1214 | 2234 | 3016 | 3036 | 50.2 | 42.9 | 4734 | 3187 | 3166 | 50.3 | 45.5 | 0.1 | 172 | 74.6 | 45.3 | 38 | 52.4 |
| 1215 | 2235 | 24559 | 24579 | 52 | 52.4 | 4735 | 25182 | 25164 | 51.4 | 47.4 | 0.6 | 624 | 76 | 41.8 | 38 | 53.7 |
| 1216 | 2236 | 1782 | 1802 | 53.3 | 47.6 | 4736 | 1881 | 1861 | 54.5 | 52.4 | 1.2 | 100 | 72.4 | 47 | 38 | 51.8 |
| 1217 | 2237 | 7880 | 7900 | 50.3 | 42.9 | 4737 | 8188 | 8169 | 50.5 | 45 | 0.1 | 309 | 74.8 | 41.7 | 38 | 52.6 |
| 1218 | 2238 | 8861 | 8880 | 50.2 | 45 | 4738 | 9248 | 9229 | 50.1 | 45 | 0 | 388 | 75 | 41 | 38 | 52.6 |
| 1219 | 2239 | 8868 | 8889 | 50.4 | 40.9 | 4739 | 9312 | 9293 | 50.6 | 45 | 0.1 | 445 | 75.3 | 41.3 | 38 | 53 |
| 1220 | 2240 | 17790 | 17813 | 54.3 | 41.7 | 4740 | 18220 | 18201 | 56.1 | 55 | 1.8 | 431 | 74.9 | 40.4 | 38 | 53.8 |
| 1221 | 2241 | 24569 | 24590 | 56.6 | 54.5 | 4741 | 25184 | 25164 | 55.9 | 52.4 | 0.7 | 616 | 75.9 | 41.7 | 38 | 55 |
| 1222 | 2242 | 13176 | 13196 | 51.4 | 47.6 | 4742 | 13328 | 13307 | 51.2 | 45.5 | 0.2 | 153 | 73.4 | 43.8 | 38 | 51.9 |
| 1223 | 2243 | 8861 | 8880 | 50.2 | 45 | 4743 | 9254 | 9236 | 50.6 | 47.4 | 0.4 | 394 | 74.9 | 40.9 | 38 | 52.6 |
| 1224 | 2244 | 24622 | 24643 | 57.1 | 54.5 | 4744 | 25400 | 25377 | 57.2 | 50 | 0.1 | 779 | 75.7 | 40.7 | 38 | 55.2 |
| 1225 | 2245 | 8868 | 8889 | 50.4 | 40.9 | 4745 | 9256 | 9237 | 50.8 | 45 | 0.4 | 389 | 75 | 41.1 | 38 | 52.7 |
| 1226 | 2246 | 3361 | 3381 | 50.5 | 42.9 | 4746 | 3497 | 3478 | 51.3 | 50 | 0.8 | 137 | 74.1 | 46.7 | 38 | 52.1 |
| 1227 | 2247 | 4593 | 4613 | 51.5 | 47.6 | 4747 | 4711 | 4693 | 50.4 | 47.4 | 1.1 | 119 | 71.5 | 42 | 38 | 50.2 |
| 1228 | 2248 | 19911 | 19930 | 50.7 | 50 | 4748 | 20615 | 20597 | 50.6 | 47.4 | 0.1 | 705 | 75.5 | 40.3 | 38 | 53.1 |
| 1229 | 2249 | 3221 | 3239 | 51.5 | 52.6 | 4749 | 3497 | 3478 | 51.3 | 50 | 0.2 | 277 | 74.6 | 41.9 | 38 | 52.7 |
| 1230 | 2250 | 3223 | 3241 | 50.2 | 52.6 | 4750 | 3504 | 3485 | 50.4 | 45 | 0.2 | 282 | 74.8 | 42.2 | 38 | 52.5 |
| 1231 | 2251 | 3223 | 3241 | 50.2 | 52.6 | 4751 | 3503 | 3484 | 51.5 | 50 | 1.2 | 281 | 74.9 | 42.3 | 38 | 52.6 |
| 1232 | 2252 | 3360 | 3380 | 51.4 | 42.9 | 4752 | 3504 | 3485 | 50.4 | 45 | 1 | 145 | 74.2 | 46.2 | 38 | 52.2 |
| 1233 | 2253 | 4593 | 4613 | 51.5 | 47.6 | 4753 | 4711 | 4692 | 51.2 | 45 | 0.3 | 119 | 71.5 | 42 | 38 | 50.5 |
| 1234 | 2254 | 4593 | 4613 | 51.5 | 47.6 | 4754 | 4710 | 4691 | 50.2 | 45 | 1.4 | 118 | 71.6 | 42.4 | 38 | 50.2 |
| 1235 | 2255 | 3016 | 3036 | 50.2 | 42.9 | 4755 | 3186 | 3165 | 50.4 | 40.9 | 0.2 | 171 | 74.4 | 45 | 38 | 52.3 |
| 1236 | 2256 | 29182 | 29206 | 55.4 | 44 | 4756 | 29301 | 29282 | 55.3 | 55 | 0.1 | 120 | 73.4 | 46.7 | 38 | 53.1 |
| 1237 | 2257 | 29183 | 29206 | 52.9 | 41.7 | 4757 | 29306 | 29287 | 54.6 | 55 | 1.7 | 124 | 73.3 | 46 | 38 | 52.3 |
| 1238 | 2258 | 29186 | 29206 | 51.3 | 42.9 | 4758 | 29298 | 29279 | 52.6 | 55 | 1.3 | 113 | 72.8 | 46 | 38 | 51.5 |
| 1239 | 2259 | 16979 | 17000 | 52.6 | 50 | 4759 | 17483 | 17465 | 54.4 | 52.6 | 1.8 | 505 | 75.9 | 42.2 | 38 | 54 |
| 1240 | 2260 | 29182 | 29205 | 54.6 | 41.7 | 4760 | 29298 | 29279 | 52.6 | 55 | 1.9 | 117 | 73.1 | 46.2 | 38 | 52 |
| 1241 | 2261 | 16981 | 17000 | 51.3 | 50 | 4761 | 17111 | 17090 | 51.1 | 40.9 | 0.2 | 131 | 74.5 | 48.1 | 38 | 52.6 |
| 1242 | 2262 | 13177 | 13197 | 50.3 | 42.9 | 4762 | 13949 | 13932 | 51.6 | 50 | 1.3 | 773 | 75.8 | 41 | 38 | 53.3 |
| 1243 | 2263 | 8867 | 8887 | 52.3 | 47.6 | 4763 | 9252 | 9234 | 51.4 | 52.6 | 0.9 | 386 | 75.1 | 41.5 | 38 | 53.1 |
| 1244 | 2264 | 24420 | 24440 | 50.8 | 42.9 | 4764 | 25081 | 25063 | 52.4 | 52.6 | 1.6 | 662 | 75.7 | 40.9 | 38 | 53.3 |
| 1245 | 2265 | 7727 | 7745 | 50.8 | 47.4 | 4765 | 8188 | 8169 | 50.5 | 45 | 0.4 | 462 | 75.6 | 41.8 | 38 | 53.1 |
| 1246 | 2266 | 2387 | 2405 | 51.6 | 52.6 | 4766 | 3055 | 3036 | 50.6 | 50 | 1.1 | 669 | 76.7 | 43.3 | 38 | 53.9 |
| 1247 | 2267 | 2671 | 2692 | 52.1 | 40.9 | 4767 | 3055 | 3036 | 50.6 | 50 | 1.5 | 385 | 74.8 | 40.5 | 38 | 52.6 |
| 1248 | 2268 | 29182 | 29202 | 51.2 | 42.9 | 4768 | 29298 | 29279 | 52.6 | 55 | 1.4 | 117 | 73.1 | 46.2 | 38 | 51.6 |
| 1249 | 2269 | 24418 | 24439 | 52.9 | 45.5 | 4769 | 25081 | 25063 | 52.4 | 52.6 | 0.5 | 664 | 75.7 | 41.1 | 38 | 53.8 |
| 1250 | 2270 | 12373 | 12391 | 50.8 | 47.4 | 4770 | 12992 | 12974 | 51.2 | 52.6 | 0.4 | 620 | 76.5 | 43.1 | 38 | 53.9 |
| 1251 | 2271 | 29179 | 29199 | 51.4 | 42.9 | 4771 | 29298 | 29279 | 52.6 | 55 | 1.2 | 120 | 73.4 | 46.7 | 38 | 51.9 |
| 1252 | 2272 | 1783 | 1803 | 54.2 | 47.6 | 4772 | 1882 | 1861 | 56 | 50 | 1.8 | 100 | 72.4 | 47 | 38 | 52 |
| 1253 | 2273 | 12373 | 12391 | 50.8 | 47.4 | 4773 | 12498 | 12480 | 50 | 47.4 | 0.7 | 126 | 72.8 | 44.4 | 38 | 51 |
| 1254 | 2274 | 7728 | 7746 | 51.7 | 52.6 | 4774 | 8190 | 8172 | 50.3 | 47.4 | 1.4 | 463 | 75.6 | 41.9 | 38 | 53.1 |
| 1255 | 2275 | 16875 | 16896 | 52.2 | 45.5 | 4775 | 17064 | 17045 | 51.4 | 50 | 0.8 | 190 | 74.5 | 44.2 | 38 | 52.7 |
| 1256 | 2276 | 1402 | 1425 | 52.8 | 41.7 | 4776 | 2103 | 2082 | 52 | 45.5 | 0.8 | 702 | 76.7 | 43.3 | 38 | 54.4 |
| 1257 | 2277 | 28971 | 28993 | 51.9 | 43.5 | 4777 | 29358 | 29339 | 52.8 | 50 | 0.9 | 388 | 76.3 | 44.3 | 38 | 54.1 |
| 1258 | 2278 | 24380 | 24399 | 55 | 55 | 4778 | 25080 | 25061 | 54.1 | 50 | 1 | 701 | 75.9 | 41.4 | 38 | 54.5 |
| 1259 | 2279 | 24380 | 24399 | 55 | 55 | 4779 | 25080 | 25062 | 53.5 | 52.6 | 1.6 | 701 | 75.9 | 41.4 | 38 | 54.3 |
| 1260 | 2280 | 3168 | 3189 | 51 | 45.5 | 4780 | 3497 | 3478 | 51.3 | 50 | 0.3 | 330 | 75.3 | 42.4 | 38 | 53.1 |
| 1261 | 2281 | 1402 | 1426 | 54.1 | 40 | 4781 | 1626 | 1602 | 56.1 | 44 | 1.9 | 225 | 76.4 | 47.6 | 38 | 54.8 |
| 1262 | 2282 | 24379 | 24398 | 55 | 55 | 4782 | 25080 | 25061 | 54.1 | 50 | 1 | 702 | 75.9 | 41.3 | 38 | 54.4 |
| 1263 | 2283 | 24379 | 24398 | 55 | 55 | 4783 | 25080 | 25062 | 53.5 | 52.6 | 1.6 | 702 | 75.9 | 41.3 | 38 | 54.3 |
| 1264 | 2284 | 16875 | 16895 | 51.6 | 47.6 | 4784 | 17062 | 17045 | 50.2 | 50 | 1.4 | 188 | 74.4 | 44.1 | 38 | 52.2 |
| 1265 | 2285 | 12726 | 12746 | 51.3 | 47.6 | 4785 | 12998 | 12979 | 50.1 | 45 | 1.2 | 273 | 75.2 | 43.2 | 38 | 52.7 |
| 1266 | 2286 | 24378 | 24397 | 55 | 55 | 4786 | 24517 | 24494 | 53.2 | 41.7 | 1.8 | 140 | 72.7 | 42.9 | 38 | 51.9 |
| 1267 | 2287 | 24378 | 24397 | 55 | 55 | 4787 | 25080 | 25061 | 54.1 | 50 | 1 | 703 | 75.9 | 41.3 | 38 | 54.4 |
| 1268 | 2288 | 28939 | 28961 | 55.2 | 47.8 | 4788 | 29306 | 29285 | 56.7 | 54.5 | 1.5 | 368 | 76.6 | 45.1 | 38 | 55.3 |
| 1269 | 2289 | 28940 | 28961 | 53.1 | 45.5 | 4789 | 29306 | 29287 | 54.6 | 55 | 1.5 | 367 | 76.5 | 45 | 38 | 54.6 |
| 1270 | 2290 | 28941 | 28961 | 51.6 | 42.9 | 4790 | 29298 | 29279 | 52.6 | 55 | 1 | 358 | 76.3 | 44.7 | 38 | 54 |
| 1271 | 2291 | 28178 | 28200 | 52 | 43.5 | 4791 | 28284 | 28265 | 52.9 | 50 | 0.9 | 107 | 74.7 | 51.4 | 38 | 53 |
| 1272 | 2292 | 28941 | 28961 | 51.6 | 42.9 | 4792 | 29358 | 29339 | 52.8 | 50 | 1.2 | 418 | 76.5 | 44.5 | 38 | 54.2 |
| 1273 | 2293 | 24378 | 24397 | 55 | 55 | 4793 | 25080 | 25062 | 53.5 | 52.6 | 1.6 | 703 | 75.9 | 41.3 | 38 | 54.2 |
| 1274 | 2294 | 28938 | 28960 | 56.1 | 47.8 | 4794 | 29306 | 29285 | 56.7 | 54.5 | 0.6 | 369 | 76.5 | 45 | 38 | 55.5 |
| 1275 | 2295 | 12234 | 12252 | 50.6 | 47.4 | 4795 | 12498 | 12480 | 50 | 47.4 | 0.5 | 265 | 74.7 | 42.3 | 38 | 52.4 |
| 1276 | 2296 | 28939 | 28960 | 54.7 | 50 | 4796 | 29306 | 29287 | 54.6 | 55 | 0.1 | 368 | 76.6 | 45.1 | 38 | 55.1 |
| 1277 | 2297 | 28140 | 28158 | 54.1 | 52.6 | 4797 | 28411 | 28393 | 52.9 | 52.6 | 1.1 | 272 | 78.8 | 52.2 | 38 | 56.2 |
| 1278 | 2298 | 28941 | 28960 | 50.9 | 45 | 4798 | 29298 | 29279 | 52.6 | 55 | 1.7 | 358 | 76.3 | 44.7 | 38 | 53.8 |
| 1279 | 2299 | 28140 | 28158 | 54.1 | 52.6 | 4799 | 28416 | 28396 | 52.4 | 47.6 | 1.7 | 277 | 78.8 | 52 | 38 | 56 |
| 1280 | 2300 | 28941 | 28960 | 50.9 | 45 | 4800 | 29358 | 29339 | 52.8 | 50 | 1.9 | 418 | 76.5 | 44.5 | 38 | 53.9 |
| 1281 | 2301 | 24179 | 24200 | 53.3 | 40.9 | 4801 | 24815 | 24791 | 54.5 | 40 | 1.2 | 637 | 75.8 | 41.3 | 38 | 54.1 |
| 1282 | 2302 | 28938 | 28956 | 50.8 | 47.4 | 4802 | 29298 | 29279 | 52.6 | 55 | 1.8 | 361 | 76.4 | 44.9 | 38 | 53.8 |
| 1283 | 2303 | 12726 | 12746 | 51.3 | 47.6 | 4803 | 12992 | 12974 | 51.2 | 52.6 | 0.1 | 267 | 75.2 | 43.4 | 38 | 53.1 |
| 1284 | 2304 | 16874 | 16893 | 52.1 | 50 | 4804 | 17062 | 17045 | 50.2 | 50 | 1.9 | 189 | 74.6 | 44.4 | 38 | 52.3 |
| 1285 | 2305 | 1352 | 1371 | 56.1 | 55 | 4805 | 1484 | 1464 | 54.3 | 47.6 | 1.8 | 133 | 74.9 | 48.9 | 38 | 53.8 |
| 1286 | 2306 | 11540 | 11561 | 53.8 | 45.5 | 4806 | 11983 | 11965 | 53 | 52.6 | 0.7 | 444 | 75.1 | 40.8 | 38 | 53.6 |
| 1287 | 2307 | 24179 | 24199 | 52.7 | 42.9 | 4807 | 24815 | 24792 | 53.4 | 41.7 | 0.7 | 637 | 75.8 | 41.3 | 38 | 53.9 |
| 1288 | 2308 | 16555 | 16572 | 50.3 | 50 | 4808 | 16777 | 16758 | 51.5 | 50 | 1.2 | 223 | 73.6 | 40.8 | 38 | 51.7 |
| 1289 | 2309 | 24178 | 24198 | 52.7 | 42.9 | 4809 | 24815 | 24791 | 54.5 | 40 | 1.8 | 638 | 75.7 | 41.2 | 38 | 53.9 |
| 1290 | 2310 | 3192 | 3213 | 51.8 | 45.5 | 4810 | 3650 | 3631 | 53.1 | 50 | 1.3 | 459 | 75.7 | 42 | 38 | 53.6 |
| 1291 | 2311 | 3192 | 3213 | 51.8 | 45.5 | 4811 | 3647 | 3628 | 50.6 | 45 | 1.2 | 456 | 75.6 | 41.9 | 38 | 53.2 |
| 1292 | 2312 | 24174 | 24195 | 52.5 | 40.9 | 4812 | 24815 | 24792 | 53.4 | 41.7 | 0.9 | 642 | 75.8 | 41.3 | 38 | 53.9 |
| 1293 | 2313 | 16553 | 16571 | 53.4 | 52.6 | 4813 | 16780 | 16760 | 51.4 | 42.9 | 2 | 228 | 73.7 | 40.8 | 38 | 52.1 |
| 1294 | 2314 | 16550 | 16568 | 54.1 | 52.6 | 4814 | 17041 | 17023 | 53.5 | 52.6 | 0.6 | 492 | 75.9 | 42.3 | 38 | 54.3 |
| 1295 | 2315 | 3192 | 3213 | 51.8 | 45.5 | 4815 | 3646 | 3625 | 52 | 40.9 | 0.2 | 455 | 75.5 | 41.8 | 38 | 53.5 |
| 1296 | 2316 | 16551 | 16568 | 51.1 | 50 | 4816 | 16777 | 16758 | 51.5 | 50 | 0.4 | 227 | 73.9 | 41.4 | 38 | 52.2 |
| 1297 | 2317 | 12373 | 12391 | 50.8 | 47.4 | 4817 | 12998 | 12979 | 50.1 | 45 | 0.7 | 626 | 76.4 | 43 | 38 | 53.6 |
| 1298 | 2318 | 28868 | 28887 | 50.7 | 45 | 4818 | 29414 | 29395 | 50.5 | 50 | 0.2 | 547 | 77 | 44.8 | 38 | 54.2 |
| 1299 | 2319 | 24028 | 24047 | 53.8 | 50 | 4819 | 24815 | 24791 | 54.5 | 40 | 0.7 | 788 | 76.3 | 42 | 38 | 54.6 |
| 1300 | 2320 | 2427 | 2445 | 52.1 | 52.6 | 4820 | 3056 | 3038 | 50.8 | 52.6 | 1.3 | 630 | 76.4 | 42.9 | 38 | 53.8 |
| 1301 | 2321 | 28867 | 28886 | 53.2 | 50 | 4821 | 29306 | 29288 | 53.5 | 52.6 | 0.3 | 440 | 76.9 | 45.2 | 38 | 54.9 |
| 1302 | 2322 | 24021 | 24044 | 52.8 | 41.7 | 4822 | 24815 | 24791 | 54.5 | 40 | 1.6 | 795 | 76.2 | 41.9 | 38 | 54.3 |
| 1303 | 2323 | 28867 | 28885 | 51.5 | 52.6 | 4823 | 29414 | 29395 | 50.5 | 50 | 0.9 | 548 | 77.1 | 44.9 | 38 | 54.2 |
| 1304 | 2324 | 12369 | 12388 | 50.6 | 45 | 4824 | 13155 | 13137 | 52.1 | 52.6 | 1.5 | 787 | 76.8 | 43.3 | 38 | 54 |
| 1305 | 2325 | 27368 | 27392 | 58.2 | 48 | 4825 | 27467 | 27443 | 59.4 | 48 | 1.2 | 100 | 71.2 | 44 | 38 | 52.4 |
| 1306 | 2326 | 27369 | 27392 | 57.2 | 50 | 4826 | 27468 | 27444 | 58.4 | 44 | 1.2 | 100 | 71.2 | 44 | 38 | 52.1 |
| 1307 | 2327 | 27369 | 27392 | 57.2 | 50 | 4827 | 27468 | 27445 | 58.1 | 45.8 | 0.8 | 100 | 71.2 | 44 | 38 | 52.1 |
| 1308 | 2328 | 23841 | 23863 | 53.7 | 47.8 | 4828 | 24022 | 24003 | 55.5 | 55 | 1.7 | 182 | 74.4 | 44.5 | 38 | 53.3 |
| 1309 | 2329 | 3192 | 3213 | 51.8 | 45.5 | 4829 | 3497 | 3478 | 51.3 | 50 | 0.5 | 306 | 75 | 42.2 | 38 | 53 |
| 1310 | 2330 | 23843 | 23863 | 50.3 | 42.9 | 4830 | 24526 | 24506 | 50.3 | 42.9 | 0 | 684 | 76.1 | 41.8 | 38 | 53.4 |
| 1311 | 2331 | 27366 | 27389 | 56.1 | 45.8 | 4831 | 27465 | 27443 | 56.4 | 47.8 | 0.3 | 100 | 71.2 | 44 | 38 | 51.8 |
| 1312 | 2332 | 27366 | 27389 | 56.1 | 45.8 | 4832 | 27465 | 27444 | 55.6 | 45.5 | 0.6 | 100 | 71.2 | 44 | 38 | 51.6 |
| 1313 | 2333 | 27366 | 27389 | 56.1 | 45.8 | 4833 | 27465 | 27445 | 55.1 | 47.6 | 1 | 100 | 71.2 | 44 | 38 | 51.5 |
| 1314 | 2334 | 16549 | 16567 | 54.9 | 52.6 | 4834 | 16779 | 16758 | 53.5 | 50 | 1.4 | 231 | 74 | 41.6 | 38 | 53 |
| 1315 | 2335 | 27369 | 27389 | 52.5 | 47.6 | 4835 | 27468 | 27448 | 53.7 | 47.6 | 1.1 | 100 | 71.2 | 44 | 38 | 50.7 |
| 1316 | 2336 | 27369 | 27389 | 52.5 | 47.6 | 4836 | 27468 | 27449 | 52.6 | 45 | 0 | 100 | 71.2 | 44 | 38 | 50.7 |
| 1317 | 2337 | 27369 | 27389 | 52.5 | 47.6 | 4837 | 27468 | 27450 | 51.9 | 47.4 | 0.7 | 100 | 71.2 | 44 | 38 | 50.5 |
| 1318 | 2338 | 28654 | 28672 | 50.6 | 52.6 | 4838 | 29412 | 29393 | 50.3 | 45 | 0.2 | 759 | 77.9 | 46.1 | 38 | 54.7 |
| 1319 | 2339 | 2429 | 2447 | 50.2 | 47.4 | 4839 | 3053 | 3034 | 50.3 | 50 | 0.1 | 625 | 76.3 | 42.6 | 39 | 53.6 |
| 1320 | 2340 | 1442 | 1461 | 51.6 | 55 | 4840 | 1697 | 1676 | 51.7 | 40.9 | 0 | 256 | 75.8 | 45.3 | 39 | 53.7 |
| 1321 | 2341 | 1442 | 1461 | 51.6 | 55 | 4841 | 1697 | 1677 | 51 | 42.9 | 0.6 | 256 | 75.8 | 45.3 | 39 | 53.5 |
| 1322 | 2342 | 1442 | 1461 | 51.6 | 55 | 4842 | 1697 | 1678 | 50.3 | 45 | 1.3 | 256 | 75.8 | 45.3 | 39 | 53.3 |
| 1323 | 2343 | 3214 | 3233 | 51.1 | 50 | 4843 | 3504 | 3485 | 50.4 | 45 | 0.7 | 291 | 74.8 | 41.9 | 39 | 52.6 |
| 1324 | 2344 | 3214 | 3233 | 51.1 | 50 | 4844 | 3503 | 3484 | 51.5 | 50 | 0.4 | 290 | 74.8 | 42.1 | 39 | 52.8 |
| 1325 | 2345 | 27374 | 27392 | 50.6 | 47.4 | 4845 | 27674 | 27653 | 52.5 | 40.9 | 1.9 | 301 | 74.1 | 40.2 | 39 | 52.2 |
| 1326 | 2346 | 9930 | 9949 | 52.2 | 50 | 4846 | 10670 | 10649 | 51.3 | 40.9 | 0.9 | 741 | 75.8 | 40.9 | 39 | 53.5 |
| 1327 | 2347 | 1442 | 1461 | 51.6 | 55 | 4847 | 2103 | 2083 | 50.6 | 42.9 | 1 | 662 | 76.7 | 43.4 | 39 | 53.9 |
| 1328 | 2348 | 8867 | 8887 | 52.3 | 47.6 | 4848 | 9375 | 9354 | 50.4 | 40.9 | 2 | 509 | 75.7 | 41.8 | 39 | 53.2 |
| 1329 | 2349 | 16367 | 16386 | 51.4 | 50 | 4849 | 16775 | 16755 | 51.1 | 42.9 | 0.3 | 409 | 75 | 40.8 | 39 | 52.9 |
| 1330 | 2350 | 18081 | 18100 | 51.7 | 50 | 4850 | 18702 | 18685 | 50.2 | 50 | 1.5 | 622 | 76.2 | 42.4 | 39 | 53.5 |
| 1331 | 2351 | 18083 | 18102 | 50.6 | 45 | 4851 | 18702 | 18685 | 50.2 | 50 | 0.4 | 620 | 76.1 | 42.3 | 39 | 53.4 |
| 1332 | 2352 | 18094 | 18113 | 51 | 50 | 4852 | 18702 | 18685 | 50.2 | 50 | 0.8 | 609 | 76.1 | 42.2 | 39 | 53.4 |
| 1333 | 2353 | 8865 | 8884 | 50.4 | 45 | 4853 | 9254 | 9236 | 50.6 | 47.4 | 0.2 | 390 | 75 | 41 | 39 | 52.7 |
| 1334 | 2354 | 16367 | 16386 | 51.4 | 50 | 4854 | 16774 | 16754 | 50.4 | 42.9 | 1 | 408 | 75 | 40.9 | 39 | 52.7 |
| 1335 | 2355 | 18008 | 18028 | 53 | 52.4 | 4855 | 18220 | 18202 | 54.8 | 52.6 | 1.9 | 213 | 74.4 | 43.2 | 39 | 53.1 |
| 1336 | 2356 | 27369 | 27389 | 52.5 | 47.6 | 4856 | 27674 | 27653 | 52.5 | 40.9 | 0.1 | 306 | 74.3 | 40.5 | 39 | 52.9 |
| 1337 | 2357 | 16367 | 16386 | 51.4 | 50 | 4857 | 16774 | 16753 | 51.1 | 40.9 | 0.3 | 408 | 75 | 40.9 | 39 | 52.9 |
| 1338 | 2358 | 1442 | 1461 | 51.6 | 55 | 4858 | 2113 | 2094 | 50.1 | 45 | 1.5 | 672 | 76.7 | 43.3 | 39 | 53.8 |
| 1339 | 2359 | 7876 | 7895 | 51.5 | 45 | 4859 | 8190 | 8172 | 50.3 | 47.4 | 1.2 | 315 | 75.1 | 42.2 | 39 | 52.7 |
| 1340 | 2360 | 18696 | 18715 | 51.7 | 50 | 4860 | 19482 | 19463 | 50.1 | 45 | 1.7 | 787 | 75.6 | 40.3 | 39 | 53 |
| 1341 | 2361 | 12370 | 12388 | 50.1 | 47.4 | 4861 | 12911 | 12892 | 50.5 | 50 | 0.4 | 542 | 76.1 | 42.4 | 39 | 53.4 |
| 1342 | 2362 | 887 | 905 | 50.1 | 47.4 | 4862 | 1493 | 1473 | 52 | 47.6 | 1.9 | 607 | 77.1 | 44.6 | 39 | 54.1 |
| 1343 | 2363 | 16367 | 16387 | 51.8 | 47.6 | 4863 | 16774 | 16751 | 53.6 | 41.7 | 1.8 | 408 | 75 | 40.9 | 39 | 53.2 |
| 1344 | 2364 | 16378 | 16397 | 50.4 | 45 | 4864 | 17111 | 17090 | 51.1 | 40.9 | 0.7 | 734 | 76.3 | 42.2 | 39 | 53.6 |
| 1345 | 2365 | 16378 | 16397 | 50.4 | 45 | 4865 | 16781 | 16761 | 51.3 | 47.6 | 0.8 | 404 | 75.1 | 41.1 | 39 | 52.8 |
| 1346 | 2366 | 1402 | 1425 | 52.8 | 41.7 | 4866 | 1501 | 1478 | 54.6 | 41.7 | 1.8 | 100 | 72 | 46 | 39 | 51.3 |
| 1347 | 2367 | 16378 | 16397 | 50.4 | 45 | 4867 | 16777 | 16758 | 51.5 | 50 | 1 | 400 | 75 | 41 | 39 | 52.7 |
| 1348 | 2368 | 16378 | 16397 | 50.4 | 45 | 4868 | 16775 | 16756 | 50.3 | 45 | 0.1 | 398 | 75 | 41 | 39 | 52.7 |
| 1349 | 2369 | 16378 | 16397 | 50.4 | 45 | 4869 | 16775 | 16755 | 51.1 | 42.9 | 0.6 | 398 | 75 | 41 | 39 | 52.7 |
| 1350 | 2370 | 16378 | 16397 | 50.4 | 45 | 4870 | 16774 | 16754 | 50.4 | 42.9 | 0 | 397 | 75 | 41.1 | 39 | 52.7 |
| 1351 | 2371 | 16378 | 16397 | 50.4 | 45 | 4871 | 16774 | 16753 | 51.1 | 40.9 | 0.7 | 397 | 75 | 41.1 | 39 | 52.8 |
| 1352 | 2372 | 16378 | 16397 | 50.4 | 45 | 4872 | 16774 | 16752 | 52.2 | 43.5 | 1.8 | 397 | 75 | 41.1 | 39 | 52.8 |
| 1353 | 2373 | 10250 | 10274 | 51.6 | 40 | 4873 | 10608 | 10589 | 51 | 50 | 0.6 | 359 | 74.6 | 40.4 | 39 | 52.6 |
| 1354 | 2374 | 16548 | 16566 | 54.9 | 52.6 | 4874 | 17112 | 17090 | 53.3 | 43.5 | 1.6 | 565 | 76.3 | 42.8 | 39 | 54.5 |
| 1355 | 2375 | 19709 | 19730 | 51.3 | 40.9 | 4875 | 19922 | 19902 | 50 | 42.9 | 1.2 | 214 | 73.8 | 41.6 | 39 | 51.8 |
| 1356 | 2376 | 3218 | 3237 | 50.5 | 45 | 4876 | 3504 | 3485 | 50.4 | 45 | 0.1 | 287 | 74.7 | 41.8 | 39 | 52.5 |
| 1357 | 2377 | 3218 | 3237 | 50.5 | 45 | 4877 | 3503 | 3484 | 51.5 | 50 | 0.9 | 286 | 74.7 | 42 | 39 | 52.6 |
| 1358 | 2378 | 19709 | 19730 | 51.3 | 40.9 | 4878 | 19920 | 19899 | 50.2 | 40.9 | 1.1 | 212 | 73.7 | 41.5 | 39 | 51.8 |
| 1359 | 2379 | 1402 | 1422 | 50.2 | 42.9 | 4879 | 1501 | 1480 | 51.9 | 40.9 | 1.7 | 100 | 72 | 46 | 39 | 50.6 |
| 1360 | 2380 | 1402 | 1422 | 50.2 | 42.9 | 4880 | 1501 | 1481 | 51.2 | 42.9 | 1.1 | 100 | 72 | 46 | 39 | 50.6 |
| 1361 | 2381 | 8867 | 8886 | 50.7 | 50 | 4881 | 9249 | 9230 | 51.5 | 45 | 0.9 | 383 | 75.2 | 41.5 | 39 | 52.9 |
| 1362 | 2382 | 19794 | 19813 | 50 | 50 | 4882 | 19928 | 19908 | 52 | 52.4 | 2 | 135 | 72.8 | 43.7 | 39 | 51.1 |
| 1363 | 2383 | 8867 | 8886 | 50.7 | 50 | 4883 | 9249 | 9231 | 50.8 | 47.4 | 0.2 | 383 | 75.2 | 41.5 | 39 | 52.9 |
| 1364 | 2384 | 9927 | 9945 | 50.8 | 52.6 | 4884 | 10183 | 10165 | 51.7 | 47.4 | 0.9 | 257 | 75.3 | 44 | 39 | 53.1 |
| 1365 | 2385 | 27366 | 27384 | 52.2 | 52.6 | 4885 | 27566 | 27546 | 50.7 | 47.6 | 1.5 | 201 | 74.7 | 44.3 | 39 | 52.6 |
| 1366 | 2386 | 9927 | 9945 | 50.8 | 52.6 | 4886 | 10183 | 10166 | 50.9 | 50 | 0.1 | 257 | 75.3 | 44 | 39 | 53.1 |
| 1367 | 2387 | 27366 | 27384 | 52.2 | 52.6 | 4887 | 27568 | 27548 | 50.2 | 42.9 | 1.9 | 203 | 74.6 | 43.8 | 39 | 52.4 |
| 1368 | 2388 | 27366 | 27384 | 52.2 | 52.6 | 4888 | 27571 | 27551 | 51.4 | 42.9 | 0.8 | 206 | 74.5 | 43.7 | 39 | 52.7 |
| 1369 | 2389 | 887 | 905 | 50.1 | 47.4 | 4889 | 1483 | 1465 | 50.5 | 47.4 | 0.4 | 597 | 77 | 44.6 | 39 | 54.1 |
| 1370 | 2390 | 27366 | 27384 | 52.2 | 52.6 | 4890 | 27579 | 27558 | 51.1 | 40.9 | 1.1 | 214 | 74.9 | 44.4 | 39 | 52.9 |
| 1371 | 2391 | 16549 | 16567 | 54.9 | 52.6 | 4891 | 16774 | 16751 | 53.6 | 41.7 | 1.3 | 226 | 74 | 41.6 | 39 | 53 |
| 1372 | 2392 | 19794 | 19813 | 50 | 50 | 4892 | 19916 | 19895 | 50.2 | 40.9 | 0.2 | 123 | 72.1 | 43.1 | 39 | 50.6 |
| 1373 | 2393 | 16551 | 16568 | 51.1 | 50 | 4893 | 17062 | 17045 | 50.2 | 50 | 0.9 | 512 | 76 | 42.4 | 39 | 53.3 |
| 1374 | 2394 | 12726 | 12746 | 51.3 | 47.6 | 4894 | 13155 | 13137 | 52.1 | 52.6 | 0.8 | 430 | 76.4 | 44 | 39 | 53.9 |
| 1375 | 2395 | 545 | 564 | 50.7 | 50 | 4895 | 1171 | 1153 | 50.4 | 47.4 | 0.3 | 627 | 78.2 | 47.2 | 39 | 54.9 |
| 1376 | 2396 | 887 | 905 | 50.1 | 47.4 | 4896 | 1483 | 1464 | 51.3 | 45 | 1.2 | 597 | 77 | 44.6 | 39 | 54.1 |
| 1377 | 2397 | 9927 | 9945 | 50.8 | 52.6 | 4897 | 10356 | 10336 | 52.4 | 47.6 | 1.6 | 430 | 75.6 | 42.1 | 39 | 53.3 |
| 1378 | 2398 | 887 | 905 | 50.1 | 47.4 | 4898 | 1481 | 1463 | 50.5 | 47.4 | 0.4 | 595 | 77 | 44.5 | 39 | 54.1 |
| 1379 | 2399 | 12726 | 12746 | 51.3 | 47.6 | 4899 | 12911 | 12891 | 51.2 | 47.6 | 0.1 | 186 | 73.5 | 41.9 | 39 | 51.9 |
| 1380 | 2400 | 19795 | 19814 | 50.4 | 45 | 4900 | 19917 | 19896 | 50.9 | 45.5 | 0.5 | 123 | 72.1 | 43.1 | 39 | 50.7 |
| 1381 | 2401 | 27361 | 27380 | 52.4 | 55 | 4901 | 27566 | 27546 | 50.7 | 47.6 | 1.7 | 206 | 75.1 | 45.1 | 39 | 52.9 |
| 1382 | 2402 | 27361 | 27380 | 52.4 | 55 | 4902 | 27569 | 27548 | 50.9 | 40.9 | 1.5 | 209 | 74.9 | 44.5 | 39 | 52.8 |
| 1383 | 2403 | 27361 | 27380 | 52.4 | 55 | 4903 | 27571 | 27551 | 51.4 | 42.9 | 1 | 211 | 75 | 44.5 | 39 | 53 |
| 1384 | 2404 | 8867 | 8886 | 50.7 | 50 | 4904 | 9256 | 9237 | 50.8 | 45 | 0.1 | 390 | 75.1 | 41.3 | 39 | 52.9 |
| 1385 | 2405 | 8373 | 8391 | 50.7 | 47.4 | 4905 | 9109 | 9087 | 50.5 | 43.5 | 0.1 | 737 | 75.4 | 40 | 39 | 53 |
| 1386 | 2406 | 19800 | 19817 | 50.4 | 50 | 4906 | 19927 | 19908 | 52.1 | 55 | 1.7 | 128 | 72.6 | 43.8 | 39 | 51 |
| 1387 | 2407 | 19800 | 19817 | 50.4 | 50 | 4907 | 19924 | 19905 | 50.1 | 50 | 0.3 | 125 | 72.2 | 43.2 | 39 | 50.7 |
| 1388 | 2408 | 16553 | 16571 | 53.4 | 52.6 | 4908 | 16774 | 16751 | 53.6 | 41.7 | 0.3 | 222 | 73.7 | 41 | 39 | 52.7 |
| 1389 | 2409 | 2427 | 2445 | 52.1 | 52.6 | 4909 | 3053 | 3034 | 50.3 | 50 | 1.8 | 627 | 76.4 | 42.7 | 39 | 53.6 |
| 1390 | 2410 | 887 | 905 | 50.1 | 47.4 | 4910 | 1479 | 1460 | 51.6 | 50 | 1.5 | 593 | 77.1 | 44.7 | 39 | 54.1 |
| 1391 | 2411 | 13177 | 13197 | 50.3 | 42.9 | 4911 | 13321 | 13301 | 50.3 | 42.9 | 0 | 145 | 73.1 | 43.4 | 39 | 51.3 |
| 1392 | 2412 | 8374 | 8395 | 52.4 | 45.5 | 4912 | 9109 | 9087 | 50.5 | 43.5 | 1.9 | 736 | 75.4 | 40.1 | 39 | 53.1 |
| 1393 | 2413 | 9926 | 9944 | 50.5 | 52.6 | 4913 | 10183 | 10165 | 51.7 | 47.4 | 1.2 | 258 | 75.2 | 43.8 | 39 | 52.9 |
| 1394 | 2414 | 16562 | 16580 | 51.9 | 52.6 | 4914 | 17056 | 17035 | 51.8 | 45.5 | 0.1 | 495 | 75.8 | 42 | 39 | 53.7 |
| 1395 | 2415 | 16562 | 16581 | 52.6 | 50 | 4915 | 17056 | 17035 | 51.8 | 45.5 | 0.8 | 495 | 75.8 | 42 | 39 | 53.7 |
| 1396 | 2416 | 9926 | 9944 | 50.5 | 52.6 | 4916 | 10183 | 10166 | 50.9 | 50 | 0.4 | 258 | 75.2 | 43.8 | 39 | 52.9 |
| 1397 | 2417 | 13177 | 13197 | 50.3 | 42.9 | 4917 | 13325 | 13305 | 50.5 | 47.6 | 0.2 | 149 | 73.3 | 43.6 | 39 | 51.5 |
| 1398 | 2418 | 10141 | 10160 | 51 | 45 | 4918 | 10356 | 10336 | 52.4 | 47.6 | 1.4 | 216 | 73.5 | 40.7 | 39 | 51.8 |
| 1399 | 2419 | 2823 | 2844 | 50.4 | 45.5 | 4919 | 3185 | 3164 | 51 | 45.5 | 0.5 | 363 | 75.6 | 42.7 | 39 | 53.1 |
| 1400 | 2420 | 19800 | 19818 | 52.1 | 52.6 | 4920 | 19916 | 19895 | 50.2 | 40.9 | 1.9 | 117 | 71.7 | 42.7 | 39 | 50.3 |
| 1401 | 2421 | 8063 | 8084 | 51.4 | 45.5 | 4921 | 8189 | 8170 | 50.6 | 50 | 0.8 | 127 | 72.3 | 43.3 | 39 | 50.9 |
| 1402 | 2422 | 985 | 1008 | 56.1 | 50 | 4922 | 1485 | 1465 | 56 | 52.4 | 0 | 501 | 76.5 | 43.7 | 39 | 55.4 |
| 1403 | 2423 | 985 | 1008 | 56.1 | 50 | 4923 | 1485 | 1466 | 55.6 | 55 | 0.5 | 501 | 76.5 | 43.7 | 39 | 55.3 |
| 1404 | 2424 | 985 | 1008 | 56.1 | 50 | 4924 | 1495 | 1474 | 55.1 | 45.5 | 1 | 511 | 76.5 | 43.6 | 39 | 55.2 |
| 1405 | 2425 | 18017 | 18036 | 54.8 | 55 | 4925 | 18231 | 18209 | 53.5 | 47.8 | 1.3 | 215 | 74.5 | 43.3 | 39 | 53.3 |
| 1406 | 2426 | 985 | 1008 | 56.1 | 50 | 4926 | 1497 | 1476 | 56.4 | 50 | 0.3 | 513 | 76.6 | 43.9 | 39 | 55.5 |
| 1407 | 2427 | 13039 | 13057 | 51.1 | 52.6 | 4927 | 13155 | 13137 | 52.1 | 52.6 | 1 | 117 | 73.4 | 47 | 39 | 51.8 |
| 1408 | 2428 | 985 | 1008 | 56.1 | 50 | 4928 | 1498 | 1478 | 54.9 | 47.6 | 1.2 | 514 | 76.5 | 43.8 | 39 | 55.1 |
| 1409 | 2429 | 988 | 1006 | 52.2 | 52.6 | 4929 | 1496 | 1478 | 50.4 | 47.4 | 1.9 | 509 | 76.5 | 43.8 | 39 | 53.8 |
| 1410 | 2430 | 988 | 1006 | 52.2 | 52.6 | 4930 | 1497 | 1480 | 50.3 | 50 | 2 | 510 | 76.6 | 43.9 | 39 | 53.8 |
| 1411 | 2431 | 19856 | 19875 | 50.2 | 45 | 4931 | 20033 | 20016 | 50.4 | 50 | 0.2 | 178 | 74.1 | 43.8 | 39 | 52 |
| 1412 | 2432 | 988 | 1006 | 52.2 | 52.6 | 4932 | 1498 | 1481 | 51 | 50 | 1.2 | 511 | 76.6 | 43.8 | 39 | 54 |
| 1413 | 2433 | 3361 | 3382 | 51.9 | 45.5 | 4933 | 3650 | 3631 | 53.1 | 50 | 1.2 | 290 | 75.7 | 44.1 | 39 | 53.6 |
| 1414 | 2434 | 8867 | 8888 | 52.7 | 45.5 | 4934 | 9365 | 9347 | 53 | 52.6 | 0.3 | 499 | 75.8 | 42.1 | 39 | 54 |
| 1415 | 2435 | 24921 | 24938 | 50.4 | 50 | 4935 | 25645 | 25626 | 50.8 | 45 | 0.4 | 725 | 75.5 | 40.4 | 39 | 53.1 |
| 1416 | 2436 | 3361 | 3382 | 51.9 | 45.5 | 4936 | 3647 | 3628 | 50.6 | 45 | 1.3 | 287 | 75.6 | 43.9 | 39 | 53.2 |
| 1417 | 2437 | 24635 | 24653 | 50.5 | 52.6 | 4937 | 25398 | 25378 | 51.1 | 42.9 | 0.6 | 764 | 75.5 | 40.3 | 39 | 53.1 |
| 1418 | 2438 | 8867 | 8888 | 52.7 | 45.5 | 4938 | 9256 | 9237 | 50.8 | 45 | 1.9 | 390 | 75.1 | 41.3 | 39 | 52.9 |
| 1419 | 2439 | 18017 | 18036 | 54.8 | 55 | 4939 | 18712 | 18693 | 54.8 | 55 | 0 | 696 | 76.4 | 42.5 | 39 | 55 |
| 1420 | 2440 | 24633 | 24651 | 50.1 | 52.6 | 4940 | 25398 | 25378 | 51.1 | 42.9 | 0.9 | 766 | 75.6 | 40.3 | 39 | 53 |
| 1421 | 2441 | 18011 | 18032 | 55.7 | 54.5 | 4941 | 18220 | 18202 | 54.8 | 52.6 | 0.9 | 210 | 74.5 | 43.3 | 39 | 53.7 |
| 1422 | 2442 | 18014 | 18032 | 51 | 52.6 | 4942 | 18223 | 18206 | 51.8 | 50 | 0.8 | 210 | 74.3 | 42.9 | 39 | 52.4 |
| 1423 | 2443 | 24630 | 24648 | 50.8 | 52.6 | 4943 | 25398 | 25378 | 51.1 | 42.9 | 0.2 | 769 | 75.6 | 40.4 | 39 | 53.3 |
| 1424 | 2444 | 18014 | 18032 | 51 | 52.6 | 4944 | 18231 | 18210 | 52.2 | 45.5 | 1.2 | 218 | 74.5 | 43.1 | 39 | 52.5 |
| 1425 | 2445 | 18014 | 18032 | 51 | 52.6 | 4945 | 18233 | 18214 | 52 | 50 | 1.1 | 220 | 74.7 | 43.6 | 39 | 52.7 |
| 1426 | 2446 | 18014 | 18032 | 51 | 52.6 | 4946 | 18233 | 18215 | 51.3 | 52.6 | 0.4 | 220 | 74.7 | 43.6 | 39 | 52.7 |
| 1427 | 2447 | 18011 | 18031 | 54.5 | 52.4 | 4947 | 18220 | 18201 | 56.1 | 55 | 1.6 | 210 | 74.5 | 43.3 | 39 | 53.6 |
| 1428 | 2448 | 3361 | 3382 | 51.9 | 45.5 | 4948 | 3646 | 3625 | 52 | 40.9 | 0.1 | 286 | 75.5 | 43.7 | 39 | 53.5 |
| 1429 | 2449 | 4658 | 4677 | 50.5 | 50 | 4949 | 5306 | 5288 | 52.4 | 52.6 | 2 | 649 | 75.5 | 40.7 | 39 | 53.1 |
| 1430 | 2450 | 18012 | 18031 | 53.2 | 55 | 4950 | 18223 | 18205 | 53.3 | 52.6 | 0.2 | 212 | 74.5 | 43.4 | 39 | 53.2 |
| 1431 | 2451 | 18012 | 18031 | 53.2 | 55 | 4951 | 18712 | 18693 | 54.8 | 55 | 1.7 | 701 | 76.4 | 42.7 | 39 | 54.6 |
| 1432 | 2452 | 13040 | 13059 | 50.9 | 50 | 4952 | 13325 | 13305 | 50.5 | 47.6 | 0.4 | 286 | 75.7 | 44.4 | 39 | 53.3 |
| 1433 | 2453 | 8867 | 8888 | 52.7 | 45.5 | 4953 | 9249 | 9231 | 50.8 | 47.4 | 1.9 | 383 | 75.2 | 41.5 | 39 | 53 |
| 1434 | 2454 | 24179 | 24198 | 51 | 45 | 4954 | 24740 | 24717 | 52.5 | 41.7 | 1.4 | 562 | 76 | 42.2 | 39 | 53.6 |
| 1435 | 2455 | 18013 | 18031 | 50.6 | 52.6 | 4955 | 18229 | 18209 | 50.1 | 42.9 | 0.5 | 217 | 74.4 | 42.9 | 39 | 52.2 |
| 1436 | 2456 | 8865 | 8884 | 50.4 | 45 | 4956 | 9340 | 9319 | 50.8 | 45.5 | 0.3 | 476 | 75.5 | 41.6 | 39 | 53.1 |
| 1437 | 2457 | 24558 | 24577 | 50.7 | 50 | 4957 | 24936 | 24919 | 51.8 | 50 | 1.1 | 379 | 75.8 | 43 | 39 | 53.3 |
| 1438 | 2458 | 8867 | 8888 | 52.7 | 45.5 | 4958 | 9249 | 9230 | 51.5 | 45 | 1.2 | 383 | 75.2 | 41.5 | 39 | 53.2 |
| 1439 | 2459 | 26039 | 26058 | 54 | 55 | 4959 | 26753 | 26733 | 54 | 52.4 | 0.1 | 715 | 76 | 41.5 | 39 | 54.5 |
| 1440 | 2460 | 26039 | 26058 | 54 | 55 | 4960 | 26753 | 26734 | 52.6 | 55 | 1.4 | 715 | 76 | 41.5 | 39 | 54.1 |
| 1441 | 2461 | 18009 | 18028 | 51.6 | 55 | 4961 | 18223 | 18206 | 51.8 | 50 | 0.1 | 215 | 74.5 | 43.3 | 39 | 52.7 |
| 1442 | 2462 | 24482 | 24503 | 51.6 | 40.9 | 4962 | 25080 | 25062 | 53.5 | 52.6 | 1.8 | 599 | 75.5 | 40.7 | 39 | 53.4 |
| 1443 | 2463 | 8861 | 8880 | 50.2 | 45 | 4963 | 9109 | 9087 | 50.5 | 43.5 | 0.4 | 249 | 73.8 | 40.6 | 39 | 51.8 |
| 1444 | 2464 | 24483 | 24503 | 51 | 42.9 | 4964 | 25086 | 25069 | 50.3 | 50 | 0.6 | 604 | 75.5 | 40.7 | 39 | 53 |
| 1445 | 2465 | 18011 | 18030 | 52.9 | 55 | 4965 | 18220 | 18202 | 54.8 | 52.6 | 2 | 210 | 74.5 | 43.3 | 39 | 53.1 |
| 1446 | 2466 | 24481 | 24502 | 51.5 | 45.5 | 4966 | 24815 | 24792 | 53.4 | 41.7 | 1.9 | 335 | 75.5 | 43 | 39 | 53.4 |
| 1447 | 2467 | 24481 | 24502 | 51.5 | 45.5 | 4967 | 25081 | 25063 | 52.4 | 52.6 | 0.9 | 601 | 75.5 | 40.8 | 39 | 53.4 |
| 1448 | 2468 | 24482 | 24502 | 50.3 | 42.9 | 4968 | 25082 | 25064 | 51.1 | 52.6 | 0.8 | 601 | 75.5 | 40.8 | 39 | 53 |
| 1449 | 2469 | 24482 | 24502 | 50.3 | 42.9 | 4969 | 25085 | 25068 | 50.3 | 50 | 0 | 604 | 75.4 | 40.6 | 39 | 53 |
| 1450 | 2470 | 24482 | 24502 | 50.3 | 42.9 | 4970 | 25086 | 25069 | 50.3 | 50 | 0 | 605 | 75.5 | 40.7 | 39 | 53 |
| 1451 | 2471 | 18011 | 18030 | 52.9 | 55 | 4971 | 18223 | 18206 | 51.8 | 50 | 1.1 | 213 | 74.4 | 43.2 | 39 | 52.7 |
| 1452 | 2472 | 18011 | 18030 | 52.9 | 55 | 4972 | 18231 | 18210 | 52.2 | 45.5 | 0.7 | 221 | 74.7 | 43.4 | 39 | 53 |
| 1453 | 2473 | 18011 | 18030 | 52.9 | 55 | 4973 | 18233 | 18214 | 52 | 50 | 0.9 | 223 | 74.9 | 43.9 | 39 | 53.1 |
| 1454 | 2474 | 18011 | 18030 | 52.9 | 55 | 4974 | 18233 | 18215 | 51.3 | 52.6 | 1.6 | 223 | 74.9 | 43.9 | 39 | 52.9 |
| 1455 | 2475 | 24419 | 24440 | 52.3 | 45.5 | 4975 | 24815 | 24792 | 53.4 | 41.7 | 1.2 | 397 | 75.9 | 43.1 | 39 | 53.9 |
| 1456 | 2476 | 18008 | 18029 | 54.5 | 50 | 4976 | 18220 | 18201 | 56.1 | 55 | 1.6 | 213 | 74.4 | 43.2 | 39 | 53.6 |
| 1457 | 2477 | 24420 | 24440 | 50.8 | 42.9 | 4977 | 24527 | 24507 | 51 | 42.9 | 0.2 | 108 | 70.7 | 41.7 | 39 | 49.9 |
| 1458 | 2478 | 12232 | 12250 | 51.9 | 52.6 | 4978 | 12994 | 12976 | 50.3 | 47.4 | 1.6 | 763 | 76.4 | 42.5 | 39 | 53.7 |
| 1459 | 2479 | 4644 | 4665 | 52.5 | 45.5 | 4979 | 5306 | 5288 | 52.4 | 52.6 | 0.1 | 663 | 75.6 | 40.9 | 39 | 53.8 |
| 1460 | 2480 | 18009 | 18029 | 53.3 | 52.4 | 4980 | 18712 | 18693 | 54.8 | 55 | 1.6 | 704 | 76.4 | 42.6 | 39 | 54.6 |
| 1461 | 2481 | 18010 | 18029 | 51.8 | 50 | 4981 | 18223 | 18205 | 53.3 | 52.6 | 1.5 | 214 | 74.4 | 43 | 39 | 52.7 |
| 1462 | 2482 | 24418 | 24439 | 52.9 | 45.5 | 4982 | 24527 | 24507 | 51 | 42.9 | 1.9 | 110 | 71.3 | 42.7 | 39 | 50.3 |
| 1463 | 2483 | 24418 | 24439 | 52.9 | 45.5 | 4983 | 24815 | 24792 | 53.4 | 41.7 | 0.5 | 398 | 75.9 | 43.2 | 39 | 54.1 |
| 1464 | 2484 | 9351 | 9370 | 51.2 | 50 | 4984 | 10017 | 9999 | 52.8 | 52.6 | 1.6 | 667 | 75.7 | 40.9 | 39 | 53.4 |
| 1465 | 2485 | 18011 | 18029 | 51.3 | 52.6 | 4985 | 18229 | 18209 | 50.1 | 42.9 | 1.2 | 219 | 74.4 | 42.9 | 39 | 52.2 |
| 1466 | 2486 | 13176 | 13196 | 51.4 | 47.6 | 4986 | 13314 | 13297 | 51 | 50 | 0.4 | 139 | 73 | 43.9 | 39 | 51.5 |
| 1467 | 2487 | 3229 | 3248 | 50.6 | 50 | 4987 | 3497 | 3478 | 51.3 | 50 | 0.6 | 269 | 74.5 | 41.6 | 39 | 52.4 |
| 1468 | 2488 | 25772 | 25793 | 52.4 | 40.9 | 4988 | 26182 | 26161 | 51.2 | 40.9 | 1.2 | 411 | 74.7 | 40.1 | 39 | 52.8 |
| 1469 | 2489 | 3229 | 3248 | 50.6 | 50 | 4989 | 3500 | 3481 | 51.2 | 50 | 0.5 | 272 | 74.5 | 41.5 | 39 | 52.4 |
| 1470 | 2490 | 13176 | 13196 | 51.4 | 47.6 | 4990 | 13323 | 13304 | 51.1 | 45 | 0.3 | 148 | 73.3 | 43.9 | 39 | 51.8 |
| 1471 | 2491 | 25771 | 25790 | 51.1 | 45 | 4991 | 26183 | 26163 | 51.7 | 42.9 | 0.6 | 413 | 74.8 | 40.4 | 39 | 52.8 |
| 1472 | 2492 | 24418 | 24436 | 50 | 47.4 | 4992 | 24526 | 24506 | 50.3 | 42.9 | 0.3 | 109 | 71.4 | 43.1 | 39 | 50.1 |
| 1473 | 2493 | 25769 | 25786 | 50.3 | 50 | 4993 | 26182 | 26161 | 51.2 | 40.9 | 0.9 | 414 | 74.8 | 40.3 | 39 | 52.6 |
| 1474 | 2494 | 18009 | 18028 | 51.6 | 55 | 4994 | 18231 | 18210 | 52.2 | 45.5 | 0.6 | 223 | 74.7 | 43.5 | 39 | 52.9 |
| 1475 | 2495 | 18009 | 18028 | 51.6 | 55 | 4995 | 18233 | 18214 | 52 | 50 | 0.4 | 225 | 74.9 | 44 | 39 | 53 |
| 1476 | 2496 | 24418 | 24436 | 50 | 47.4 | 4996 | 25082 | 25064 | 51.1 | 52.6 | 1.1 | 665 | 75.8 | 41.2 | 39 | 53.2 |
| 1477 | 2497 | 18009 | 18028 | 51.6 | 55 | 4997 | 18233 | 18215 | 51.3 | 52.6 | 0.3 | 225 | 74.9 | 44 | 39 | 53 |
| 1478 | 2498 | 24418 | 24436 | 50 | 47.4 | 4998 | 25209 | 25190 | 50.6 | 50 | 0.6 | 792 | 76.2 | 41.9 | 39 | 53.5 |
| 1479 | 2499 | 25363 | 25381 | 51.1 | 52.6 | 4999 | 25650 | 25631 | 51.3 | 45 | 0.1 | 288 | 74.2 | 40.6 | 39 | 52.4 |
| 1480 | 2500 | 25363 | 25381 | 51.1 | 52.6 | 5000 | 25651 | 25634 | 50.4 | 50 | 0.7 | 289 | 74.3 | 40.8 | 39 | 52.2 |
| 1481 | 2501 | 25354 | 25372 | 50.9 | 52.6 | 5001 | 25548 | 25531 | 51.1 | 50 | 0.2 | 195 | 74.1 | 43.1 | 39 | 52.2 |
| 1482 | 2502 | 18005 | 18024 | 51.1 | 50 | 5002 | 18223 | 18206 | 51.8 | 50 | 0.6 | 219 | 74.4 | 42.9 | 39 | 52.5 |
| 1483 | 2503 | 18005 | 18024 | 51.1 | 50 | 5003 | 18231 | 18210 | 52.2 | 45.5 | 1.1 | 227 | 74.6 | 43.2 | 39 | 52.7 |
| 1484 | 2504 | 25354 | 25372 | 50.9 | 52.6 | 5004 | 25651 | 25632 | 52.7 | 50 | 1.8 | 298 | 74.6 | 41.3 | 39 | 52.6 |
| 1485 | 2505 | 18005 | 18024 | 51.1 | 50 | 5005 | 18233 | 18215 | 51.3 | 52.6 | 0.2 | 229 | 74.9 | 43.7 | 39 | 52.8 |
| 1486 | 2506 | 18003 | 18023 | 53.5 | 52.4 | 5006 | 18712 | 18693 | 54.8 | 55 | 1.3 | 710 | 76.4 | 42.7 | 39 | 54.7 |
| 1487 | 2507 | 13176 | 13196 | 51.4 | 47.6 | 5007 | 13326 | 13306 | 50.7 | 42.9 | 0.7 | 151 | 73.4 | 43.7 | 39 | 51.7 |
| 1488 | 2508 | 8868 | 8889 | 50.4 | 40.9 | 5008 | 9311 | 9292 | 50.7 | 50 | 0.3 | 444 | 75.4 | 41.4 | 39 | 53 |
| 1489 | 2509 | 25354 | 25372 | 50.9 | 52.6 | 5009 | 25832 | 25811 | 52.1 | 50 | 1.2 | 479 | 75 | 40.3 | 39 | 52.9 |
| 1490 | 2510 | 8375 | 8396 | 51.8 | 45.5 | 5010 | 9109 | 9087 | 50.5 | 43.5 | 1.2 | 735 | 75.4 | 40 | 39 | 53 |
| 1491 | 2511 | 9918 | 9938 | 51.4 | 47.6 | 5011 | 10017 | 9999 | 52.8 | 52.6 | 1.3 | 100 | 72.4 | 47 | 39 | 51.2 |
| 1492 | 2512 | 8375 | 8396 | 51.8 | 45.5 | 5012 | 8933 | 8916 | 52.2 | 50 | 0.4 | 559 | 75.1 | 40.1 | 39 | 53.2 |
| 1493 | 2513 | 17840 | 17859 | 50.8 | 45 | 5013 | 18632 | 18611 | 50.2 | 40.9 | 0.6 | 793 | 76.1 | 41.5 | 39 | 53.4 |
| 1494 | 2514 | 13040 | 13059 | 50.9 | 50 | 5014 | 13155 | 13138 | 50.4 | 50 | 0.5 | 116 | 73.2 | 46.6 | 39 | 51.4 |
| 1495 | 2515 | 25348 | 25366 | 51.2 | 47.4 | 5015 | 25650 | 25631 | 51.3 | 45 | 0.1 | 303 | 74.6 | 41.3 | 39 | 52.7 |
| 1496 | 2516 | 25348 | 25366 | 51.2 | 47.4 | 5016 | 25651 | 25634 | 50.4 | 50 | 0.7 | 304 | 74.7 | 41.4 | 39 | 52.5 |
| 1497 | 2517 | 13040 | 13059 | 50.9 | 50 | 5017 | 13178 | 13157 | 50.4 | 40.9 | 0.5 | 139 | 73.6 | 45.3 | 39 | 51.7 |
| 1498 | 2518 | 17792 | 17813 | 51.6 | 40.9 | 5018 | 18223 | 18205 | 53.3 | 52.6 | 1.7 | 432 | 74.9 | 40.3 | 39 | 53 |
| 1499 | 2519 | 25348 | 25366 | 51.2 | 47.4 | 5019 | 25832 | 25811 | 52.1 | 50 | 0.9 | 485 | 75.1 | 40.4 | 39 | 53 |
| 1500 | 2520 | 25348 | 25366 | 51.2 | 47.4 | 5020 | 25833 | 25812 | 51.4 | 45.5 | 0.2 | 486 | 75 | 40.3 | 39 | 53 |
| 1501 | 2521 | 25347 | 25365 | 52 | 52.6 | 5021 | 25651 | 25632 | 52.7 | 50 | 0.7 | 305 | 74.8 | 41.6 | 39 | 53 |
| 1502 | 2522 | 8868 | 8889 | 50.4 | 40.9 | 5022 | 9252 | 9234 | 51.4 | 52.6 | 1 | 385 | 75.1 | 41.3 | 39 | 52.8 |
| 1503 | 2523 | 17793 | 17813 | 50 | 42.9 | 5023 | 18229 | 18209 | 50.1 | 42.9 | 0.1 | 437 | 74.9 | 40.3 | 39 | 52.5 |
| 1504 | 2524 | 17793 | 17813 | 50 | 42.9 | 5024 | 18231 | 18211 | 50.6 | 47.6 | 0.6 | 439 | 75 | 40.5 | 39 | 52.6 |
| 1505 | 2525 | 17793 | 17813 | 50 | 42.9 | 5025 | 18234 | 18216 | 51 | 52.6 | 1 | 442 | 75.1 | 40.7 | 39 | 52.7 |
| 1506 | 2526 | 17793 | 17813 | 50 | 42.9 | 5026 | 18238 | 18219 | 50.3 | 45 | 0.2 | 446 | 75.1 | 40.8 | 39 | 52.7 |
| 1507 | 2527 | 17793 | 17813 | 50 | 42.9 | 5027 | 18239 | 18220 | 50 | 45 | 0 | 447 | 75.1 | 40.7 | 39 | 52.7 |
| 1508 | 2528 | 24180 | 24199 | 50.3 | 40 | 5028 | 24938 | 24921 | 50.4 | 50 | 0.1 | 759 | 75.8 | 40.8 | 39 | 53.2 |
| 1509 | 2529 | 25348 | 25365 | 50.4 | 50 | 5029 | 25832 | 25811 | 52.1 | 50 | 1.7 | 485 | 75.1 | 40.4 | 39 | 52.8 |
| 1510 | 2530 | 25068 | 25085 | 50.3 | 50 | 5030 | 25182 | 25164 | 51.4 | 47.4 | 1.1 | 115 | 73.3 | 47 | 39 | 51.5 |
| 1511 | 2531 | 29260 | 29278 | 51.3 | 47.4 | 5031 | 29414 | 29395 | 50.5 | 50 | 0.8 | 155 | 74.3 | 45.8 | 39 | 52.3 |
| 1512 | 2532 | 24179 | 24198 | 51 | 45 | 5032 | 24933 | 24913 | 51.1 | 42.9 | 0.1 | 755 | 75.8 | 40.9 | 39 | 53.5 |
| 1513 | 2533 | 17790 | 17811 | 51.6 | 40.9 | 5033 | 18223 | 18205 | 53.3 | 52.6 | 1.7 | 434 | 74.9 | 40.3 | 39 | 53 |
| 1514 | 2534 | 8063 | 8084 | 51.4 | 45.5 | 5034 | 8190 | 8172 | 50.3 | 47.4 | 1.1 | 128 | 72.2 | 43 | 39 | 50.8 |
| 1515 | 2535 | 24178 | 24197 | 50.3 | 40 | 5035 | 24936 | 24919 | 51.8 | 50 | 1.5 | 759 | 75.8 | 41 | 39 | 53.2 |
| 1516 | 2536 | 17791 | 17811 | 50 | 42.9 | 5036 | 18229 | 18209 | 50.1 | 42.9 | 0.1 | 439 | 74.9 | 40.3 | 39 | 52.5 |
| 1517 | 2537 | 17791 | 17811 | 50 | 42.9 | 5037 | 18231 | 18211 | 50.6 | 47.6 | 0.6 | 441 | 75 | 40.6 | 39 | 52.6 |
| 1518 | 2538 | 24174 | 24194 | 50.9 | 42.9 | 5038 | 24740 | 24717 | 52.5 | 41.7 | 1.5 | 567 | 76 | 42.2 | 39 | 53.6 |
| 1519 | 2539 | 24174 | 24194 | 50.9 | 42.9 | 5039 | 24933 | 24913 | 51.1 | 42.9 | 0.2 | 760 | 75.8 | 40.9 | 39 | 53.4 |
| 1520 | 2540 | 17791 | 17811 | 50 | 42.9 | 5040 | 18234 | 18216 | 51 | 52.6 | 1 | 444 | 75.1 | 40.8 | 39 | 52.7 |
| 1521 | 2541 | 17791 | 17811 | 50 | 42.9 | 5041 | 18238 | 18219 | 50.3 | 45 | 0.2 | 448 | 75.1 | 40.8 | 39 | 52.7 |
| 1522 | 2542 | 17791 | 17811 | 50 | 42.9 | 5042 | 18239 | 18220 | 50 | 45 | 0 | 449 | 75.1 | 40.8 | 39 | 52.7 |
| 1523 | 2543 | 24035 | 24053 | 52.2 | 52.6 | 5043 | 24526 | 24506 | 50.3 | 42.9 | 1.9 | 492 | 75.4 | 41.3 | 39 | 53 |
| 1524 | 2544 | 24035 | 24053 | 52.2 | 52.6 | 5044 | 24527 | 24507 | 51 | 42.9 | 1.2 | 493 | 75.4 | 41.2 | 39 | 53.2 |
| 1525 | 2545 | 17607 | 17628 | 52.3 | 40.9 | 5045 | 18231 | 18209 | 53.5 | 47.8 | 1.2 | 625 | 75.2 | 40 | 39 | 53.4 |
| 1526 | 2546 | 29196 | 29216 | 52.5 | 47.6 | 5046 | 29358 | 29339 | 52.8 | 50 | 0.3 | 163 | 74.9 | 46.6 | 39 | 53.3 |
| 1527 | 2547 | 17608 | 17628 | 50.9 | 42.9 | 5047 | 18231 | 18211 | 50.6 | 47.6 | 0.4 | 624 | 75.2 | 40.1 | 39 | 52.9 |
| 1528 | 2548 | 8868 | 8889 | 50.4 | 40.9 | 5048 | 9248 | 9229 | 50.1 | 45 | 0.3 | 381 | 75 | 41.2 | 39 | 52.7 |
| 1529 | 2549 | 17608 | 17628 | 50.9 | 42.9 | 5049 | 18234 | 18216 | 51 | 52.6 | 0 | 627 | 75.3 | 40.2 | 39 | 53.1 |
| 1530 | 2550 | 29196 | 29215 | 51.8 | 50 | 5050 | 29358 | 29339 | 52.8 | 50 | 1 | 163 | 74.9 | 46.6 | 39 | 53.1 |
| 1531 | 2551 | 24023 | 24044 | 51.4 | 40.9 | 5051 | 24527 | 24508 | 50.5 | 45 | 0.9 | 505 | 75.4 | 41 | 39 | 53 |
| 1532 | 2552 | 9409 | 9428 | 51.6 | 45 | 5052 | 9989 | 9968 | 51 | 40.9 | 0.6 | 581 | 75.3 | 40.4 | 39 | 53.1 |
| 1533 | 2553 | 29196 | 29214 | 51.1 | 52.6 | 5053 | 29358 | 29339 | 52.8 | 50 | 1.7 | 163 | 74.9 | 46.6 | 39 | 52.8 |
| 1534 | 2554 | 8861 | 8880 | 50.2 | 45 | 5054 | 9257 | 9238 | 50.5 | 45 | 0.3 | 397 | 74.9 | 40.8 | 39 | 52.6 |
| 1535 | 2555 | 17607 | 17627 | 51.6 | 42.9 | 5055 | 18231 | 18209 | 53.5 | 47.8 | 1.9 | 625 | 75.2 | 40 | 39 | 53.2 |
| 1536 | 2556 | 29195 | 29213 | 51.9 | 52.6 | 5056 | 29358 | 29339 | 52.8 | 50 | 0.9 | 164 | 74.8 | 46.3 | 39 | 53 |
| 1537 | 2557 | 17608 | 17627 | 50.2 | 45 | 5057 | 18231 | 18211 | 50.6 | 47.6 | 0.4 | 624 | 75.2 | 40.1 | 39 | 52.8 |
| 1538 | 2558 | 985 | 1004 | 51.1 | 50 | 5058 | 1622 | 1602 | 51.6 | 47.6 | 0.5 | 638 | 77.2 | 44.7 | 39 | 54.4 |
| 1539 | 2559 | 17608 | 17627 | 50.2 | 45 | 5059 | 18234 | 18216 | 51 | 52.6 | 0.8 | 627 | 75.3 | 40.2 | 39 | 52.9 |
| 1540 | 2560 | 23841 | 23860 | 52.1 | 55 | 5060 | 24496 | 24478 | 50.7 | 52.6 | 1.4 | 656 | 76.1 | 42.1 | 39 | 53.6 |
| 1541 | 2561 | 23841 | 23860 | 52.1 | 55 | 5061 | 24498 | 24479 | 51.2 | 50 | 0.9 | 658 | 76.1 | 42.1 | 39 | 53.8 |
| 1542 | 2562 | 3404 | 3422 | 50.5 | 47.4 | 5062 | 3647 | 3628 | 50.6 | 45 | 0.1 | 244 | 74.6 | 42.6 | 39 | 52.5 |
| 1543 | 2563 | 9349 | 9367 | 51.7 | 52.6 | 5063 | 10017 | 9999 | 52.8 | 52.6 | 1.1 | 669 | 75.7 | 41 | 39 | 53.6 |
| 1544 | 2564 | 23841 | 23859 | 50.5 | 52.6 | 5064 | 24496 | 24478 | 50.7 | 52.6 | 0.2 | 656 | 76.1 | 42.1 | 39 | 53.5 |
| 1545 | 2565 | 25068 | 25085 | 50.3 | 50 | 5065 | 25548 | 25531 | 51.1 | 50 | 0.8 | 481 | 75.8 | 42.2 | 39 | 53.3 |
| 1546 | 2566 | 29186 | 29205 | 50.1 | 40 | 5066 | 29414 | 29395 | 50.5 | 50 | 0.4 | 229 | 75.4 | 45 | 39 | 52.9 |
| 1547 | 2567 | 29182 | 29204 | 53.2 | 43.5 | 5067 | 29358 | 29339 | 52.8 | 50 | 0.4 | 177 | 74.6 | 45.2 | 39 | 53.2 |
| 1548 | 2568 | 23841 | 23859 | 50.5 | 52.6 | 5068 | 24498 | 24479 | 51.2 | 50 | 0.7 | 658 | 76.1 | 42.1 | 39 | 53.5 |
| 1549 | 2569 | 3404 | 3422 | 50.5 | 47.4 | 5069 | 3646 | 3625 | 52 | 40.9 | 1.5 | 243 | 74.5 | 42.4 | 39 | 52.4 |
| 1550 | 2570 | 29183 | 29204 | 50.4 | 40.9 | 5070 | 29414 | 29395 | 50.5 | 50 | 0.2 | 232 | 75.4 | 44.8 | 39 | 53 |
| 1551 | 2571 | 23841 | 23859 | 50.5 | 52.6 | 5071 | 24527 | 24508 | 50.5 | 45 | 0 | 687 | 76.1 | 41.9 | 39 | 53.5 |
| 1552 | 2572 | 23838 | 23857 | 50.4 | 50 | 5072 | 24093 | 24075 | 50.9 | 52.6 | 0.5 | 256 | 75.8 | 45.3 | 39 | 53.3 |
| 1553 | 2573 | 23838 | 23857 | 50.4 | 50 | 5073 | 24496 | 24478 | 50.7 | 52.6 | 0.3 | 659 | 76.1 | 41.9 | 39 | 53.4 |
| 1554 | 2574 | 23838 | 23857 | 50.4 | 50 | 5074 | 24498 | 24479 | 51.2 | 50 | 0.8 | 661 | 76.1 | 41.9 | 39 | 53.5 |
| 1555 | 2575 | 29181 | 29201 | 52.4 | 47.6 | 5075 | 29358 | 29339 | 52.8 | 50 | 0.4 | 178 | 74.8 | 45.5 | 39 | 53.2 |
| 1556 | 2576 | 29181 | 29201 | 52.4 | 47.6 | 5076 | 29414 | 29395 | 50.5 | 50 | 1.9 | 234 | 75.6 | 45.3 | 39 | 53.2 |
| 1557 | 2577 | 29180 | 29200 | 51.7 | 42.9 | 5077 | 29358 | 29339 | 52.8 | 50 | 1.2 | 179 | 74.7 | 45.3 | 39 | 52.9 |
| 1558 | 2578 | 985 | 1004 | 51.1 | 50 | 5078 | 1498 | 1481 | 51 | 50 | 0.1 | 514 | 76.5 | 43.8 | 39 | 54 |
| 1559 | 2579 | 985 | 1004 | 51.1 | 50 | 5079 | 1497 | 1480 | 50.3 | 50 | 0.8 | 513 | 76.6 | 43.9 | 39 | 53.8 |
| 1560 | 2580 | 8859 | 8879 | 50 | 42.9 | 5080 | 9254 | 9236 | 50.6 | 47.4 | 0.6 | 396 | 75 | 40.9 | 39 | 52.6 |
| 1561 | 2581 | 29178 | 29198 | 51.4 | 42.9 | 5081 | 29414 | 29395 | 50.5 | 50 | 0.9 | 237 | 75.6 | 45.1 | 39 | 53.2 |
| 1562 | 2582 | 8859 | 8879 | 50 | 42.9 | 5082 | 9340 | 9319 | 50.8 | 45.5 | 0.7 | 482 | 75.5 | 41.5 | 39 | 53 |
| 1563 | 2583 | 16909 | 16928 | 50.8 | 45 | 5083 | 17109 | 17089 | 50.4 | 42.9 | 0.4 | 201 | 74.9 | 44.8 | 39 | 52.7 |
| 1564 | 2584 | 8794 | 8813 | 51.6 | 45 | 5084 | 8919 | 8901 | 50.4 | 47.4 | 1.2 | 126 | 71.8 | 42.1 | 39 | 50.5 |
| 1565 | 2585 | 8794 | 8813 | 51.6 | 45 | 5085 | 8920 | 8902 | 52.8 | 52.6 | 1.2 | 127 | 72 | 42.5 | 39 | 51 |
| 1566 | 2586 | 985 | 1004 | 51.1 | 50 | 5086 | 1496 | 1478 | 50.4 | 47.4 | 0.7 | 512 | 76.5 | 43.8 | 39 | 53.8 |
| 1567 | 2587 | 18017 | 18036 | 54.8 | 55 | 5087 | 18223 | 18205 | 53.3 | 52.6 | 1.5 | 207 | 74.3 | 43 | 39 | 53.1 |
| 1568 | 2588 | 4593 | 4613 | 51.5 | 47.6 | 5088 | 4994 | 4974 | 51.2 | 47.6 | 0.3 | 402 | 76.1 | 43.5 | 39 | 53.7 |
| 1569 | 2589 | 18017 | 18036 | 54.8 | 55 | 5089 | 18220 | 18201 | 56.1 | 55 | 1.3 | 204 | 74.3 | 43.1 | 39 | 53.5 |
| 1570 | 2590 | 6155 | 6174 | 52.1 | 50 | 5090 | 6486 | 6467 | 50.8 | 45 | 1.2 | 332 | 74.5 | 40.7 | 39 | 52.5 |
| 1571 | 2591 | 6158 | 6178 | 51.3 | 42.9 | 5091 | 6486 | 6467 | 50.8 | 45 | 0.4 | 329 | 74.3 | 40.1 | 39 | 52.4 |
| 1572 | 2592 | 3232 | 3251 | 50.3 | 50 | 5092 | 3500 | 3481 | 51.2 | 50 | 0.8 | 269 | 74.5 | 41.6 | 39 | 52.3 |
| 1573 | 2593 | 3232 | 3251 | 50.3 | 50 | 5093 | 3497 | 3478 | 51.3 | 50 | 1 | 266 | 74.5 | 41.7 | 39 | 52.3 |
| 1574 | 2594 | 28523 | 28544 | 51.6 | 40.9 | 5094 | 29298 | 29279 | 52.6 | 55 | 1 | 776 | 78.4 | 47.3 | 39 | 52.3 |
| 1575 | 2595 | 28965 | 28984 | 52.9 | 55 | 5095 | 29358 | 29339 | 52.8 | 50 | 0.1 | 394 | 76.6 | 44.9 | 39 | 54.6 |
| 1576 | 2596 | 8866 | 8885 | 51.1 | 45 | 5096 | 9256 | 9237 | 50.8 | 45 | 0.3 | 391 | 75.1 | 41.2 | 39 | 52.9 |
| 1577 | 2597 | 28518 | 28538 | 51.2 | 42.9 | 5097 | 28672 | 28654 | 50.6 | 52.6 | 0.7 | 155 | 76.7 | 51.6 | 39 | 54 |
| 1578 | 2598 | 6165 | 6183 | 51.2 | 52.6 | 5098 | 6486 | 6467 | 50.8 | 45 | 0.3 | 322 | 74.5 | 40.7 | 39 | 52.5 |
| 1579 | 2599 | 6264 | 6283 | 50.4 | 50 | 5099 | 6483 | 6463 | 50.2 | 42.9 | 0.2 | 220 | 73.8 | 41.4 | 39 | 51.8 |
| 1580 | 2600 | 18074 | 18093 | 50.3 | 45 | 5100 | 18233 | 18215 | 51.3 | 52.6 | 1 | 160 | 73.9 | 44.4 | 39 | 51.9 |
| 1581 | 2601 | 6271 | 6291 | 51.1 | 47.6 | 5101 | 6483 | 6463 | 50.2 | 42.9 | 0.9 | 213 | 73.5 | 40.8 | 39 | 51.6 |
| 1582 | 2602 | 18074 | 18093 | 50.3 | 45 | 5102 | 18231 | 18210 | 52.2 | 45.5 | 1.9 | 158 | 73.5 | 43.7 | 39 | 51.7 |
| 1583 | 2603 | 6274 | 6293 | 50.1 | 45 | 5103 | 6483 | 6463 | 50.2 | 42.9 | 0.1 | 210 | 73.5 | 41 | 39 | 51.6 |
| 1584 | 2604 | 18074 | 18093 | 50.3 | 45 | 5104 | 18223 | 18206 | 51.8 | 50 | 1.5 | 150 | 73.2 | 43.3 | 39 | 51.4 |
| 1585 | 2605 | 5 | 23 | 51.3 | 52.6 | 5105 | 314 | 296 | 50.6 | 47.4 | 0.6 | 310 | 76.8 | 46.5 | 39 | 54 |
| 1586 | 2606 | 6343 | 6364 | 50.7 | 45.5 | 5106 | 6486 | 6467 | 50.8 | 45 | 0.1 | 144 | 71.7 | 40.3 | 39 | 50.5 |
| 1587 | 2607 | 3800 | 3820 | 50.6 | 42.9 | 5107 | 4445 | 4425 | 50.6 | 42.9 | 0 | 646 | 75.4 | 40.2 | 39 | 53 |
| 1588 | 2608 | 7615 | 7635 | 51.1 | 47.6 | 5108 | 7821 | 7798 | 52.8 | 41.7 | 1.6 | 207 | 73.7 | 41.5 | 39 | 52 |
| 1589 | 2609 | 7723 | 7741 | 52.2 | 52.6 | 5109 | 8049 | 8032 | 50.4 | 50 | 1.8 | 327 | 74.9 | 41.6 | 39 | 52.6 |
| 1590 | 2610 | 1 | 19 | 50.1 | 52.6 | 5110 | 314 | 296 | 50.6 | 47.4 | 0.6 | 314 | 76.8 | 46.5 | 39 | 53.9 |
| 1591 | 2611 | 7725 | 7742 | 50 | 50 | 5111 | 7856 | 7836 | 51.1 | 42.9 | 1.1 | 132 | 71.3 | 40.2 | 39 | 50 |
| 1592 | 2612 | 18074 | 18094 | 51.1 | 42.9 | 5112 | 18233 | 18215 | 51.3 | 52.6 | 0.3 | 160 | 73.9 | 44.4 | 39 | 52.1 |
| 1593 | 2613 | 3168 | 3189 | 51 | 45.5 | 5113 | 3503 | 3484 | 51.5 | 50 | 0.5 | 336 | 75.3 | 42.6 | 39 | 53.1 |
| 1594 | 2614 | 18074 | 18094 | 51.1 | 42.9 | 5114 | 18231 | 18210 | 52.2 | 45.5 | 1.1 | 158 | 73.5 | 43.7 | 39 | 51.9 |
| 1595 | 2615 | 13177 | 13197 | 50.3 | 42.9 | 5115 | 13312 | 13294 | 51 | 52.6 | 0.7 | 136 | 72.7 | 43.4 | 39 | 51.1 |
| 1596 | 2616 | 28190 | 28209 | 54.2 | 55 | 5116 | 28671 | 28652 | 52.8 | 55 | 1.5 | 482 | 79.9 | 52.1 | 39 | 56.8 |
| 1597 | 2617 | 28190 | 28209 | 54.2 | 55 | 5117 | 28673 | 28654 | 53.5 | 55 | 0.7 | 484 | 79.9 | 52.3 | 39 | 57.1 |
| 1598 | 2618 | 28190 | 28208 | 51.7 | 52.6 | 5118 | 28671 | 28652 | 52.8 | 55 | 1 | 482 | 79.9 | 52.1 | 39 | 56.5 |
| 1599 | 2619 | 28190 | 28208 | 51.7 | 52.6 | 5119 | 28671 | 28653 | 50.2 | 52.6 | 1.5 | 482 | 79.9 | 52.1 | 39 | 56.1 |
| 1600 | 2620 | 18074 | 18094 | 51.1 | 42.9 | 5120 | 18223 | 18206 | 51.8 | 50 | 0.7 | 150 | 73.2 | 43.3 | 39 | 51.6 |
| 1601 | 2621 | 28185 | 28205 | 53.5 | 47.6 | 5121 | 28284 | 28265 | 52.9 | 50 | 0.6 | 100 | 74.5 | 52 | 39 | 53.1 |
| 1602 | 2622 | 28187 | 28205 | 53.1 | 52.6 | 5122 | 28672 | 28653 | 51.8 | 55 | 1.2 | 486 | 79.9 | 52.3 | 39 | 56.6 |
| 1603 | 2623 | 2371 | 2389 | 50.3 | 47.4 | 5123 | 2900 | 2881 | 50.1 | 45 | 0.2 | 530 | 76.8 | 44.3 | 39 | 53.9 |
| 1604 | 2624 | 2371 | 2389 | 50.3 | 47.4 | 5124 | 3052 | 3033 | 50.3 | 50 | 0.1 | 682 | 76.7 | 43.4 | 39 | 53.9 |
| 1605 | 2625 | 2371 | 2389 | 50.3 | 47.4 | 5125 | 3056 | 3038 | 50.8 | 52.6 | 0.5 | 686 | 76.7 | 43.4 | 39 | 53.9 |
| 1606 | 2626 | 18074 | 18095 | 52.2 | 45.5 | 5126 | 18223 | 18205 | 53.3 | 52.6 | 1.1 | 150 | 73.2 | 43.3 | 39 | 52 |
| 1607 | 2627 | 2220 | 2239 | 51.3 | 45 | 5127 | 2891 | 2873 | 50.8 | 47.4 | 0.5 | 672 | 76.8 | 43.8 | 39 | 54.1 |
| 1608 | 2628 | 18077 | 18097 | 51.5 | 47.6 | 5128 | 18662 | 18641 | 50.4 | 40.9 | 1.1 | 586 | 76.2 | 42.7 | 39 | 53.6 |
| 1609 | 2629 | 28117 | 28135 | 50.6 | 52.6 | 5129 | 28671 | 28653 | 50.2 | 52.6 | 0.4 | 555 | 80 | 51.9 | 39 | 56.1 |
| 1610 | 2630 | 28116 | 28134 | 50.8 | 47.4 | 5130 | 28671 | 28653 | 50.2 | 52.6 | 0.6 | 556 | 79.9 | 51.8 | 39 | 56.1 |
| 1611 | 2631 | 12232 | 12250 | 51.9 | 52.6 | 5131 | 13000 | 12981 | 51.1 | 45 | 0.8 | 769 | 76.5 | 42.5 | 39 | 54 |
| 1612 | 2632 | 18080 | 18098 | 51.2 | 52.6 | 5132 | 18702 | 18685 | 50.2 | 50 | 1 | 623 | 76.2 | 42.4 | 39 | 53.5 |
| 1613 | 2633 | 12232 | 12250 | 51.9 | 52.6 | 5133 | 12999 | 12980 | 50.6 | 40 | 1.4 | 768 | 76.4 | 42.4 | 39 | 53.8 |
| 1614 | 2634 | 28820 | 28840 | 54.8 | 47.6 | 5134 | 29306 | 29285 | 56.7 | 54.5 | 1.9 | 487 | 77.1 | 45.4 | 39 | 55.5 |
| 1615 | 2635 | 28820 | 28840 | 54.8 | 47.6 | 5135 | 29306 | 29287 | 54.6 | 55 | 0.3 | 487 | 77.1 | 45.4 | 39 | 55.5 |
| 1616 | 2636 | 18080 | 18098 | 51.2 | 52.6 | 5136 | 18642 | 18622 | 50.5 | 42.9 | 0.7 | 563 | 76.2 | 42.6 | 39 | 53.6 |
| 1617 | 2637 | 8865 | 8884 | 50.4 | 45 | 5137 | 9249 | 9231 | 50.8 | 47.4 | 0.4 | 385 | 75.1 | 41.3 | 39 | 52.8 |
| 1618 | 2638 | 8865 | 8884 | 50.4 | 45 | 5138 | 9249 | 9230 | 51.5 | 45 | 1.1 | 385 | 75.1 | 41.3 | 39 | 52.8 |
| 1619 | 2639 | 8865 | 8884 | 50.4 | 45 | 5139 | 9109 | 9087 | 50.5 | 43.5 | 0.1 | 245 | 73.9 | 40.8 | 39 | 51.9 |
| 1620 | 2640 | 28819 | 28839 | 56.6 | 52.4 | 5140 | 29306 | 29285 | 56.7 | 54.5 | 0.2 | 488 | 77.2 | 45.5 | 39 | 56.1 |
| 1621 | 2641 | 9130 | 9151 | 52 | 40.9 | 5141 | 9364 | 9346 | 53.9 | 52.6 | 2 | 235 | 74.8 | 43.4 | 39 | 53.1 |
| 1622 | 2642 | 28820 | 28839 | 54.3 | 50 | 5142 | 29306 | 29287 | 54.6 | 55 | 0.3 | 487 | 77.1 | 45.4 | 39 | 55.4 |
| 1623 | 2643 | 8865 | 8884 | 50.4 | 45 | 5143 | 9248 | 9229 | 50.1 | 45 | 0.3 | 384 | 75 | 41.1 | 39 | 52.7 |
| 1624 | 2644 | 18080 | 18098 | 51.2 | 52.6 | 5144 | 18229 | 18209 | 50.1 | 42.9 | 1.1 | 150 | 73.2 | 43.3 | 39 | 51.4 |
| 1625 | 2645 | 15752 | 15772 | 50.8 | 47.6 | 5145 | 16175 | 16155 | 51.8 | 47.6 | 1 | 424 | 75.1 | 41 | 39 | 52.9 |
| 1626 | 2646 | 12232 | 12250 | 51.9 | 52.6 | 5146 | 12498 | 12480 | 50 | 47.4 | 1.9 | 267 | 74.7 | 42.3 | 39 | 52.4 |
| 1627 | 2647 | 18078 | 18098 | 51.5 | 47.6 | 5147 | 18223 | 18205 | 53.3 | 52.6 | 1.8 | 146 | 73 | 43.2 | 39 | 51.6 |
| 1628 | 2648 | 7833 | 7853 | 50.7 | 47.6 | 5148 | 8054 | 8035 | 50.4 | 50 | 0.2 | 222 | 74.6 | 43.2 | 39 | 52.4 |
| 1629 | 2649 | 230 | 248 | 51.2 | 52.6 | 5149 | 713 | 695 | 50.7 | 47.4 | 0.5 | 484 | 79.3 | 50.6 | 39 | 55.8 |
| 1630 | 2650 | 1472 | 1491 | 51.2 | 45 | 5150 | 2153 | 2134 | 50.4 | 45 | 0.8 | 682 | 76.5 | 42.8 | 39 | 53.7 |
| 1631 | 2651 | 18076 | 18098 | 54.4 | 47.8 | 5151 | 18220 | 18201 | 56.1 | 55 | 1.8 | 145 | 73.1 | 43.4 | 39 | 52.6 |
| 1632 | 2652 | 1442 | 1461 | 51.6 | 55 | 5152 | 1694 | 1673 | 51.7 | 40.9 | 0.1 | 253 | 75.9 | 45.5 | 39 | 53.7 |
| 1633 | 2653 | 28618 | 28636 | 52.5 | 52.6 | 5153 | 29358 | 29339 | 52.8 | 50 | 0.3 | 741 | 78.2 | 47 | 39 | 55.6 |
| 1634 | 2654 | 940 | 959 | 56.3 | 55 | 5154 | 1701 | 1677 | 54.7 | 40 | 1.6 | 762 | 77.1 | 44.2 | 40 | 55.5 |
| 1635 | 2655 | 18076 | 18097 | 53.1 | 45.5 | 5155 | 18696 | 18672 | 53.9 | 40 | 0.8 | 621 | 76.2 | 42.4 | 40 | 54.4 |
| 1636 | 2656 | 940 | 959 | 56.3 | 55 | 5156 | 1697 | 1673 | 54.4 | 40 | 1.9 | 758 | 77.2 | 44.3 | 40 | 55.5 |
| 1637 | 2657 | 3016 | 3036 | 50.2 | 42.9 | 5157 | 3188 | 3167 | 50.2 | 40.9 | 0.1 | 173 | 74.5 | 45.1 | 40 | 52.3 |
| 1638 | 2658 | 18077 | 18097 | 51.5 | 47.6 | 5158 | 18696 | 18673 | 53.4 | 41.7 | 1.9 | 620 | 76.2 | 42.4 | 40 | 53.9 |
| 1639 | 2659 | 18077 | 18097 | 51.5 | 47.6 | 5159 | 18697 | 18679 | 51.9 | 52.6 | 0.3 | 621 | 76.2 | 42.5 | 40 | 53.9 |
| 1640 | 2660 | 9352 | 9371 | 50.6 | 45 | 5160 | 9989 | 9968 | 51 | 40.9 | 0.4 | 638 | 75.4 | 40.3 | 40 | 53 |
| 1641 | 2661 | 6042 | 6062 | 50.4 | 47.6 | 5161 | 6374 | 6353 | 50 | 40.9 | 0.3 | 333 | 74.6 | 40.8 | 40 | 52.3 |
| 1642 | 2662 | 942 | 960 | 52.1 | 52.6 | 5162 | 1697 | 1678 | 50.3 | 45 | 1.8 | 756 | 77.2 | 44.3 | 40 | 54.2 |
| 1643 | 2663 | 942 | 960 | 52.1 | 52.6 | 5163 | 1697 | 1677 | 51 | 42.9 | 1.1 | 756 | 77.2 | 44.3 | 40 | 54.4 |
| 1644 | 2664 | 6042 | 6062 | 50.4 | 47.6 | 5164 | 6292 | 6273 | 50.8 | 45 | 0.4 | 251 | 73.9 | 40.6 | 40 | 51.9 |
| 1645 | 2665 | 942 | 960 | 52.1 | 52.6 | 5165 | 1697 | 1676 | 51.7 | 40.9 | 0.5 | 756 | 77.2 | 44.3 | 40 | 54.6 |
| 1646 | 2666 | 942 | 960 | 52.1 | 52.6 | 5166 | 1694 | 1673 | 51.7 | 40.9 | 0.4 | 753 | 77.2 | 44.4 | 40 | 54.7 |
| 1647 | 2667 | 13176 | 13196 | 51.4 | 47.6 | 5167 | 13749 | 13727 | 50.5 | 43.5 | 0.9 | 574 | 76.2 | 42.7 | 40 | 53.6 |
| 1648 | 2668 | 13176 | 13196 | 51.4 | 47.6 | 5168 | 13949 | 13932 | 51.6 | 50 | 0.2 | 774 | 75.9 | 41.1 | 40 | 53.6 |
| 1649 | 2669 | 942 | 960 | 52.1 | 52.6 | 5169 | 1493 | 1473 | 52 | 47.6 | 0.1 | 552 | 76.9 | 44.4 | 40 | 54.5 |
| 1650 | 2670 | 6042 | 6062 | 50.4 | 47.6 | 5170 | 6292 | 6272 | 51.5 | 42.9 | 1.1 | 251 | 73.9 | 40.6 | 40 | 51.9 |
| 1651 | 2671 | 6042 | 6062 | 50.4 | 47.6 | 5171 | 6290 | 6270 | 50.9 | 42.9 | 0.6 | 249 | 73.8 | 40.6 | 40 | 51.9 |
| 1652 | 2672 | 6042 | 6062 | 50.4 | 47.6 | 5172 | 6289 | 6267 | 52.2 | 43.5 | 1.8 | 248 | 73.9 | 40.7 | 40 | 51.9 |
| 1653 | 2673 | 9402 | 9420 | 51.3 | 47.4 | 5173 | 10017 | 9999 | 52.8 | 52.6 | 1.4 | 616 | 75.6 | 41.1 | 40 | 53.5 |
| 1654 | 2674 | 943 | 961 | 50.3 | 47.4 | 5174 | 1697 | 1678 | 50.3 | 45 | 0 | 755 | 77.1 | 44.2 | 40 | 54.2 |
| 1655 | 2675 | 9139 | 9159 | 52.5 | 47.6 | 5175 | 9324 | 9300 | 52.9 | 40 | 0.4 | 186 | 73.9 | 43 | 40 | 52.6 |
| 1656 | 2676 | 9139 | 9159 | 52.5 | 47.6 | 5176 | 9324 | 9301 | 52.4 | 41.7 | 0.1 | 186 | 73.9 | 43 | 40 | 52.6 |
| 1657 | 2677 | 943 | 961 | 50.3 | 47.4 | 5177 | 1697 | 1677 | 51 | 42.9 | 0.7 | 755 | 77.1 | 44.2 | 40 | 54.2 |
| 1658 | 2678 | 6222 | 6246 | 52.2 | 40 | 5178 | 6486 | 6467 | 50.8 | 45 | 1.4 | 265 | 73.8 | 40 | 40 | 52 |
| 1659 | 2679 | 3895 | 3914 | 50.3 | 45 | 5179 | 4608 | 4590 | 51.5 | 52.6 | 1.2 | 714 | 75.5 | 40.3 | 40 | 53 |
| 1660 | 2680 | 3889 | 3911 | 54.2 | 47.8 | 5180 | 4610 | 4590 | 53.2 | 52.4 | 1.1 | 722 | 75.5 | 40.4 | 40 | 53.9 |
| 1661 | 2681 | 3889 | 3908 | 51.3 | 50 | 5181 | 4608 | 4590 | 51.5 | 52.6 | 0.3 | 720 | 75.5 | 40.4 | 40 | 53.4 |
| 1662 | 2682 | 9139 | 9159 | 52.5 | 47.6 | 5182 | 9359 | 9335 | 54.5 | 40 | 1.9 | 221 | 74.7 | 43.4 | 40 | 53.1 |
| 1663 | 2683 | 943 | 961 | 50.3 | 47.4 | 5183 | 1697 | 1676 | 51.7 | 40.9 | 1.4 | 755 | 77.1 | 44.2 | 40 | 54.2 |
| 1664 | 2684 | 943 | 961 | 50.3 | 47.4 | 5184 | 1694 | 1673 | 51.7 | 40.9 | 1.5 | 752 | 77.2 | 44.3 | 40 | 54.2 |
| 1665 | 2685 | 6302 | 6321 | 51.4 | 50 | 5185 | 6483 | 6463 | 50.2 | 42.9 | 1.2 | 182 | 72.9 | 40.7 | 40 | 51.2 |
| 1666 | 2686 | 9409 | 9428 | 51.6 | 45 | 5186 | 10017 | 9999 | 52.8 | 52.6 | 1.2 | 609 | 75.6 | 41.1 | 40 | 53.5 |
| 1667 | 2687 | 943 | 961 | 50.3 | 47.4 | 5187 | 1493 | 1473 | 52 | 47.6 | 1.7 | 551 | 76.8 | 44.3 | 40 | 54 |
| 1668 | 2688 | 13039 | 13058 | 51.8 | 50 | 5188 | 13312 | 13294 | 51 | 52.6 | 0.8 | 274 | 75.7 | 44.5 | 40 | 53.4 |
| 1669 | 2689 | 3799 | 3820 | 52.9 | 45.5 | 5189 | 4565 | 4542 | 53.9 | 41.7 | 1 | 767 | 75.5 | 40.2 | 40 | 53.8 |
| 1670 | 2690 | 985 | 1004 | 51.1 | 50 | 5190 | 1481 | 1463 | 50.5 | 47.4 | 0.6 | 497 | 76.4 | 43.5 | 40 | 53.7 |
| 1671 | 2691 | 13039 | 13058 | 51.8 | 50 | 5191 | 13325 | 13305 | 50.5 | 47.6 | 1.3 | 287 | 75.8 | 44.6 | 40 | 53.3 |
| 1672 | 2692 | 7615 | 7635 | 51.1 | 47.6 | 5192 | 8049 | 8032 | 50.4 | 50 | 0.8 | 435 | 75.7 | 42.3 | 40 | 53.2 |
| 1673 | 2693 | 7615 | 7635 | 51.1 | 47.6 | 5193 | 7853 | 7833 | 50.7 | 47.6 | 0.4 | 239 | 74.4 | 42.3 | 40 | 52.4 |
| 1674 | 2694 | 3034 | 3053 | 50.3 | 50 | 5194 | 3503 | 3484 | 51.5 | 50 | 1.2 | 470 | 76.3 | 43.4 | 40 | 53.6 |
| 1675 | 2695 | 9140 | 9159 | 50.1 | 45 | 5195 | 9334 | 9315 | 52.1 | 50 | 2 | 195 | 74.3 | 43.6 | 40 | 52.1 |
| 1676 | 2696 | 3799 | 3819 | 51.3 | 47.6 | 5196 | 4186 | 4168 | 51.8 | 52.6 | 0.5 | 388 | 75.3 | 41.8 | 40 | 53.2 |
| 1677 | 2697 | 3799 | 3819 | 51.3 | 47.6 | 5197 | 4434 | 4416 | 51.5 | 52.6 | 0.2 | 636 | 75.3 | 40.3 | 40 | 53.2 |
| 1678 | 2698 | 3799 | 3819 | 51.3 | 47.6 | 5198 | 4435 | 4417 | 50.5 | 52.6 | 0.8 | 637 | 75.4 | 40.3 | 40 | 53 |
| 1679 | 2699 | 7617 | 7636 | 50.9 | 50 | 5199 | 8190 | 8172 | 50.3 | 47.4 | 0.6 | 574 | 76.1 | 42.3 | 40 | 53.4 |
| 1680 | 2700 | 18011 | 18032 | 55.7 | 54.5 | 5200 | 18443 | 18424 | 55.9 | 55 | 0.2 | 433 | 76.1 | 43.2 | 40 | 55.1 |
| 1681 | 2701 | 18013 | 18032 | 52.2 | 55 | 5201 | 18696 | 18672 | 53.9 | 40 | 1.7 | 684 | 76.3 | 42.4 | 40 | 54.2 |
| 1682 | 2702 | 18013 | 18032 | 52.2 | 55 | 5202 | 18696 | 18673 | 53.4 | 41.7 | 1.2 | 684 | 76.3 | 42.4 | 40 | 54.2 |
| 1683 | 2703 | 13177 | 13197 | 50.3 | 42.9 | 5203 | 13545 | 13527 | 50.3 | 52.6 | 0 | 369 | 77 | 46.1 | 40 | 54.1 |
| 1684 | 2704 | 9922 | 9941 | 51.3 | 50 | 5204 | 10455 | 10434 | 51.1 | 40.9 | 0.1 | 534 | 75.3 | 40.6 | 40 | 53.1 |
| 1685 | 2705 | 7617 | 7636 | 50.9 | 50 | 5205 | 7853 | 7833 | 50.7 | 47.6 | 0.3 | 237 | 74.4 | 42.2 | 40 | 52.4 |
| 1686 | 2706 | 13177 | 13197 | 50.3 | 42.9 | 5206 | 13329 | 13308 | 50.5 | 40.9 | 0.2 | 153 | 73.2 | 43.1 | 40 | 51.4 |
| 1687 | 2707 | 18014 | 18032 | 51 | 52.6 | 5207 | 18238 | 18219 | 50.3 | 45 | 0.7 | 225 | 74.8 | 43.6 | 40 | 52.5 |
| 1688 | 2708 | 18014 | 18032 | 51 | 52.6 | 5208 | 18239 | 18220 | 50 | 45 | 0.9 | 226 | 74.7 | 43.4 | 40 | 52.4 |
| 1689 | 2709 | 18014 | 18032 | 51 | 52.6 | 5209 | 18697 | 18679 | 51.9 | 52.6 | 0.9 | 684 | 76.3 | 42.4 | 40 | 53.8 |
| 1690 | 2710 | 7708 | 7730 | 50.6 | 43.5 | 5210 | 7853 | 7833 | 50.7 | 47.6 | 0.1 | 146 | 71.8 | 40.4 | 40 | 50.6 |
| 1691 | 2711 | 9140 | 9159 | 50.1 | 45 | 5211 | 9358 | 9338 | 51 | 42.9 | 0.9 | 219 | 74.4 | 42.9 | 40 | 52.2 |
| 1692 | 2712 | 7723 | 7741 | 52.2 | 52.6 | 5212 | 7856 | 7836 | 51.1 | 42.9 | 1.1 | 134 | 71.4 | 40.3 | 40 | 50.4 |
| 1693 | 2713 | 988 | 1006 | 52.2 | 52.6 | 5213 | 1171 | 1153 | 50.4 | 47.4 | 1.8 | 184 | 73.8 | 42.9 | 40 | 51.9 |
| 1694 | 2714 | 13177 | 13197 | 50.3 | 42.9 | 5214 | 13328 | 13307 | 51.2 | 45.5 | 0.9 | 152 | 73.3 | 43.4 | 40 | 51.5 |
| 1695 | 2715 | 9935 | 9955 | 50.4 | 42.9 | 5215 | 10608 | 10589 | 51 | 50 | 0.6 | 674 | 75.8 | 41.1 | 40 | 53.2 |
| 1696 | 2716 | 985 | 1008 | 56.1 | 50 | 5216 | 1484 | 1463 | 55.5 | 50 | 0.5 | 500 | 76.4 | 43.6 | 40 | 55.3 |
| 1697 | 2717 | 13033 | 13051 | 52.1 | 52.6 | 5217 | 13179 | 13158 | 50.4 | 40.9 | 1.7 | 147 | 74.3 | 46.3 | 40 | 52.2 |
| 1698 | 2718 | 12977 | 12996 | 50.2 | 40 | 5218 | 13320 | 13300 | 51.4 | 47.6 | 1.1 | 344 | 76.1 | 44.2 | 40 | 53.4 |
| 1699 | 2719 | 12977 | 12996 | 50.2 | 40 | 5219 | 13321 | 13301 | 50.3 | 42.9 | 0.1 | 345 | 76 | 44.1 | 40 | 53.4 |
| 1700 | 2720 | 2823 | 2844 | 50.4 | 45.5 | 5220 | 3192 | 3171 | 51.9 | 50 | 1.5 | 370 | 75.7 | 43 | 40 | 53.2 |
| 1701 | 2721 | 18009 | 18030 | 54.6 | 54.5 | 5221 | 18443 | 18424 | 55.9 | 55 | 1.4 | 435 | 76.1 | 43.2 | 40 | 54.7 |
| 1702 | 2722 | 12976 | 12995 | 51.1 | 45 | 5222 | 13320 | 13300 | 51.4 | 47.6 | 0.3 | 345 | 76.1 | 44.3 | 40 | 53.7 |
| 1703 | 2723 | 1046 | 1064 | 51.2 | 47.4 | 5223 | 1531 | 1512 | 52.7 | 55 | 1.5 | 486 | 76.7 | 44.2 | 40 | 54.1 |
| 1704 | 2724 | 12976 | 12995 | 51.1 | 45 | 5224 | 13321 | 13301 | 50.3 | 42.9 | 0.8 | 346 | 76.1 | 44.2 | 40 | 53.5 |
| 1705 | 2725 | 12976 | 12994 | 50.3 | 47.4 | 5225 | 13320 | 13300 | 51.4 | 47.6 | 1.1 | 345 | 76.1 | 44.3 | 40 | 53.5 |
| 1706 | 2726 | 12976 | 12994 | 50.3 | 47.4 | 5226 | 13321 | 13301 | 50.3 | 42.9 | 0 | 346 | 76.1 | 44.2 | 40 | 53.5 |
| 1707 | 2727 | 18011 | 18030 | 52.9 | 55 | 5227 | 18696 | 18672 | 53.9 | 40 | 1 | 686 | 76.3 | 42.4 | 40 | 54.4 |
| 1708 | 2728 | 18011 | 18030 | 52.9 | 55 | 5228 | 18696 | 18673 | 53.4 | 41.7 | 0.5 | 686 | 76.3 | 42.4 | 40 | 54.4 |
| 1709 | 2729 | 18011 | 18030 | 52.9 | 55 | 5229 | 18697 | 18679 | 51.9 | 52.6 | 1 | 687 | 76.3 | 42.5 | 40 | 54.1 |
| 1710 | 2730 | 9140 | 9159 | 50.1 | 45 | 5230 | 9374 | 9353 | 50.1 | 40.9 | 0 | 235 | 74.5 | 42.6 | 40 | 52.3 |
| 1711 | 2731 | 3 | 23 | 55.4 | 52.4 | 5231 | 204 | 185 | 56.6 | 55 | 1.3 | 202 | 75 | 45 | 40 | 54.2 |
| 1712 | 2732 | 15255 | 15273 | 50.3 | 52.6 | 5232 | 15761 | 15741 | 51.7 | 47.6 | 1.4 | 507 | 75 | 40 | 40 | 52.7 |
| 1713 | 2733 | 15255 | 15273 | 50.3 | 52.6 | 5233 | 15763 | 15743 | 52 | 47.6 | 1.7 | 509 | 75 | 40.1 | 40 | 52.7 |
| 1714 | 2734 | 12965 | 12985 | 51.2 | 42.9 | 5234 | 13320 | 13300 | 51.4 | 47.6 | 0.2 | 356 | 76.1 | 44.1 | 40 | 53.7 |
| 1715 | 2735 | 8373 | 8391 | 50.7 | 47.4 | 5235 | 9060 | 9039 | 50.3 | 40.9 | 0.4 | 688 | 75.4 | 40.1 | 40 | 53 |
| 1716 | 2736 | 12962 | 12980 | 50.7 | 47.4 | 5236 | 13320 | 13300 | 51.4 | 47.6 | 0.7 | 359 | 76.2 | 44.3 | 40 | 53.6 |
| 1717 | 2737 | 12938 | 12957 | 50.9 | 45 | 5237 | 13155 | 13137 | 52.1 | 52.6 | 1.2 | 218 | 75.4 | 45.4 | 40 | 53.2 |
| 1718 | 2738 | 2671 | 2692 | 52.1 | 40.9 | 5238 | 3190 | 3169 | 50.7 | 45.5 | 1.5 | 520 | 75.6 | 41.5 | 40 | 53.2 |
| 1719 | 2739 | 2671 | 2692 | 52.1 | 40.9 | 5239 | 3192 | 3171 | 51.9 | 50 | 0.2 | 522 | 75.7 | 41.8 | 40 | 53.7 |
| 1720 | 2740 | 12938 | 12956 | 50.1 | 47.4 | 5240 | 13155 | 13137 | 52.1 | 52.6 | 2 | 218 | 75.4 | 45.4 | 40 | 52.9 |
| 1721 | 2741 | 26421 | 26441 | 51.5 | 42.9 | 5241 | 26592 | 26574 | 52.4 | 52.6 | 0.9 | 172 | 72.4 | 40.1 | 40 | 51.2 |
| 1722 | 2742 | 18006 | 18028 | 54.5 | 52.2 | 5242 | 18443 | 18424 | 55.9 | 55 | 1.4 | 438 | 76.1 | 43.2 | 40 | 54.7 |
| 1723 | 2743 | 26421 | 26441 | 51.5 | 42.9 | 5243 | 26656 | 26635 | 52.9 | 45.5 | 1.4 | 236 | 74.2 | 41.9 | 40 | 52.5 |
| 1724 | 2744 | 3055 | 3074 | 51.1 | 50 | 5244 | 3210 | 3190 | 50.5 | 47.6 | 0.6 | 156 | 74.2 | 45.5 | 40 | 52.2 |
| 1725 | 2745 | 7833 | 7853 | 50.7 | 47.6 | 5245 | 8189 | 8170 | 50.6 | 50 | 0 | 357 | 75.6 | 42.9 | 40 | 53.2 |
| 1726 | 2746 | 26421 | 26441 | 51.5 | 42.9 | 5246 | 26658 | 26640 | 50.8 | 47.4 | 0.7 | 238 | 74.1 | 41.6 | 40 | 52.2 |
| 1727 | 2747 | 9131 | 9151 | 50.4 | 42.9 | 5247 | 9328 | 9310 | 51 | 52.6 | 0.7 | 198 | 74.3 | 43.4 | 40 | 52.2 |
| 1728 | 2748 | 24921 | 24938 | 50.4 | 50 | 5248 | 25650 | 25631 | 51.3 | 45 | 0.9 | 730 | 75.5 | 40.4 | 40 | 53.1 |
| 1729 | 2749 | 24921 | 24938 | 50.4 | 50 | 5249 | 25651 | 25634 | 50.4 | 50 | 0 | 731 | 75.6 | 40.5 | 40 | 53.1 |
| 1730 | 2750 | 9130 | 9151 | 52 | 40.9 | 5250 | 9324 | 9301 | 52.4 | 41.7 | 0.5 | 195 | 73.9 | 42.6 | 40 | 52.4 |
| 1731 | 2751 | 9130 | 9151 | 52 | 40.9 | 5251 | 9324 | 9300 | 52.9 | 40 | 1 | 195 | 73.9 | 42.6 | 40 | 52.4 |
| 1732 | 2752 | 8376 | 8396 | 50.6 | 42.9 | 5252 | 9107 | 9086 | 51.6 | 45.5 | 1 | 732 | 75.4 | 40 | 40 | 53.1 |
| 1733 | 2753 | 11541 | 11561 | 50.9 | 42.9 | 5253 | 11727 | 11708 | 50.4 | 45 | 0.5 | 187 | 73 | 40.6 | 40 | 51.3 |
| 1734 | 2754 | 11540 | 11561 | 53.8 | 45.5 | 5254 | 11984 | 11966 | 53 | 52.6 | 0.7 | 445 | 75.1 | 40.7 | 40 | 53.6 |
| 1735 | 2755 | 2371 | 2389 | 50.3 | 47.4 | 5255 | 2672 | 2654 | 50.9 | 52.6 | 0.5 | 302 | 77.1 | 47.4 | 40 | 54.2 |
| 1736 | 2756 | 2371 | 2389 | 50.3 | 47.4 | 5256 | 2998 | 2977 | 51.1 | 40.9 | 0.8 | 628 | 76.7 | 43.5 | 40 | 53.9 |
| 1737 | 2757 | 11543 | 11562 | 50.4 | 40 | 5257 | 11727 | 11708 | 50.4 | 45 | 0.1 | 185 | 72.9 | 40.5 | 40 | 51.2 |
| 1738 | 2758 | 26040 | 26061 | 56.4 | 54.5 | 5258 | 26589 | 26567 | 56.1 | 47.8 | 0.4 | 550 | 75.1 | 40 | 40 | 54.5 |
| 1739 | 2759 | 11541 | 11562 | 51.5 | 40.9 | 5259 | 11984 | 11966 | 53 | 52.6 | 1.5 | 444 | 75 | 40.5 | 40 | 53.1 |
| 1740 | 2760 | 7728 | 7746 | 51.7 | 52.6 | 5260 | 8187 | 8167 | 50.4 | 42.9 | 1.3 | 460 | 75.6 | 42 | 40 | 53.2 |
| 1741 | 2761 | 26040 | 26061 | 56.4 | 54.5 | 5261 | 26657 | 26634 | 54.6 | 41.7 | 1.9 | 618 | 75.5 | 40.6 | 40 | 54.3 |
| 1742 | 2762 | 2223 | 2243 | 50.2 | 42.9 | 5262 | 2675 | 2656 | 50.4 | 50 | 0.2 | 453 | 77 | 45.3 | 40 | 54 |
| 1743 | 2763 | 2220 | 2239 | 51.3 | 45 | 5263 | 2676 | 2657 | 50.7 | 50 | 0.5 | 457 | 76.9 | 45.1 | 40 | 54.1 |
| 1744 | 2764 | 11541 | 11560 | 50.1 | 45 | 5264 | 11727 | 11707 | 51.1 | 42.9 | 1 | 187 | 73 | 40.6 | 40 | 51.2 |
| 1745 | 2765 | 24559 | 24580 | 54.2 | 54.5 | 5265 | 25088 | 25070 | 54.5 | 52.6 | 0.2 | 530 | 75.5 | 41.1 | 40 | 54.2 |
| 1746 | 2766 | 12233 | 12251 | 51.1 | 52.6 | 5266 | 12998 | 12979 | 50.1 | 45 | 1 | 766 | 76.5 | 42.6 | 40 | 53.7 |
| 1747 | 2767 | 12233 | 12251 | 51.1 | 52.6 | 5267 | 12412 | 12392 | 50 | 42.9 | 1.1 | 180 | 73.2 | 41.7 | 40 | 51.4 |
| 1748 | 2768 | 24562 | 24580 | 50.1 | 52.6 | 5268 | 25086 | 25069 | 50.3 | 50 | 0.2 | 525 | 75.4 | 41 | 40 | 52.9 |
| 1749 | 2769 | 9931 | 9950 | 50.2 | 45 | 5269 | 10608 | 10589 | 51 | 50 | 0.8 | 678 | 75.8 | 41.2 | 40 | 53.2 |
| 1750 | 2770 | 24562 | 24580 | 50.1 | 52.6 | 5270 | 25209 | 25188 | 52 | 45.5 | 1.9 | 648 | 76.1 | 42 | 40 | 53.4 |
| 1751 | 2771 | 24562 | 24580 | 50.1 | 52.6 | 5271 | 25209 | 25189 | 51.4 | 47.6 | 1.3 | 648 | 76.1 | 42 | 40 | 53.4 |
| 1752 | 2772 | 3789 | 3807 | 51.8 | 52.6 | 5272 | 4445 | 4425 | 50.6 | 42.9 | 1.2 | 657 | 75.5 | 40.5 | 40 | 53.1 |
| 1753 | 2773 | 26039 | 26058 | 54 | 55 | 5273 | 26656 | 26634 | 53.4 | 43.5 | 0.6 | 618 | 75.5 | 40.6 | 40 | 53.9 |
| 1754 | 2774 | 26039 | 26058 | 54 | 55 | 5274 | 26660 | 26639 | 52.5 | 45.5 | 1.5 | 622 | 75.4 | 40.5 | 40 | 53.7 |
| 1755 | 2775 | 3789 | 3807 | 51.8 | 52.6 | 5275 | 4444 | 4424 | 50.6 | 42.9 | 1.2 | 656 | 75.5 | 40.5 | 40 | 53.1 |
| 1756 | 2776 | 24559 | 24579 | 52 | 52.4 | 5276 | 25086 | 25069 | 50.3 | 50 | 1.6 | 528 | 75.5 | 41.1 | 40 | 53 |
| 1757 | 2777 | 12235 | 12253 | 50.1 | 52.6 | 5277 | 12999 | 12980 | 50.6 | 40 | 0.5 | 765 | 76.4 | 42.5 | 40 | 53.6 |
| 1758 | 2778 | 24559 | 24579 | 52 | 52.4 | 5278 | 25209 | 25188 | 52 | 45.5 | 0 | 651 | 76.1 | 42.1 | 40 | 54 |
| 1759 | 2779 | 24559 | 24579 | 52 | 52.4 | 5279 | 25209 | 25189 | 51.4 | 47.6 | 0.6 | 651 | 76.1 | 42.1 | 40 | 53.8 |
| 1760 | 2780 | 887 | 905 | 50.1 | 47.4 | 5280 | 1499 | 1482 | 50.1 | 50 | 0.1 | 613 | 77.1 | 44.7 | 40 | 54.1 |
| 1761 | 2781 | 24558 | 24577 | 50.7 | 50 | 5281 | 25182 | 25164 | 51.4 | 47.4 | 0.7 | 625 | 76 | 41.9 | 40 | 53.5 |
| 1762 | 2782 | 26039 | 26058 | 54 | 55 | 5282 | 26828 | 26810 | 52.9 | 52.6 | 1.2 | 790 | 76.4 | 42.4 | 40 | 54.5 |
| 1763 | 2783 | 8866 | 8885 | 51.1 | 45 | 5283 | 9597 | 9577 | 50.3 | 42.9 | 0.8 | 732 | 75.8 | 41.1 | 40 | 53.3 |
| 1764 | 2784 | 7727 | 7745 | 50.8 | 47.4 | 5284 | 8049 | 8032 | 50.4 | 50 | 0.5 | 323 | 74.8 | 41.5 | 40 | 52.6 |
| 1765 | 2785 | 13177 | 13197 | 50.3 | 42.9 | 5285 | 13747 | 13726 | 50.8 | 40.9 | 0.4 | 571 | 76.2 | 42.6 | 40 | 53.5 |
| 1766 | 2786 | 887 | 905 | 50.1 | 47.4 | 5286 | 1498 | 1481 | 51 | 50 | 0.9 | 612 | 77.2 | 44.8 | 40 | 54.1 |
| 1767 | 2787 | 1784 | 1803 | 52.5 | 50 | 5287 | 2103 | 2083 | 50.6 | 42.9 | 2 | 320 | 76 | 44.4 | 40 | 53.5 |
| 1768 | 2788 | 12235 | 12253 | 50.1 | 52.6 | 5288 | 12498 | 12480 | 50 | 47.4 | 0.1 | 264 | 74.7 | 42.4 | 40 | 52.4 |
| 1769 | 2789 | 1784 | 1802 | 51.8 | 52.6 | 5289 | 2103 | 2083 | 50.6 | 42.9 | 1.3 | 320 | 76 | 44.4 | 40 | 53.5 |
| 1770 | 2790 | 887 | 905 | 50.1 | 47.4 | 5290 | 1496 | 1478 | 50.4 | 47.4 | 0.3 | 610 | 77.1 | 44.8 | 40 | 54.1 |
| 1771 | 2791 | 1783 | 1801 | 52.9 | 52.6 | 5291 | 2153 | 2133 | 52.1 | 42.9 | 0.9 | 371 | 76 | 43.7 | 40 | 53.9 |
| 1772 | 2792 | 887 | 905 | 50.1 | 47.4 | 5292 | 1494 | 1476 | 50.7 | 47.4 | 0.6 | 608 | 77.1 | 44.7 | 40 | 54.1 |
| 1773 | 2793 | 3791 | 3809 | 51.8 | 52.6 | 5293 | 4445 | 4425 | 50.6 | 42.9 | 1.2 | 655 | 75.5 | 40.5 | 40 | 53.1 |
| 1774 | 2794 | 17813 | 17832 | 50.1 | 45 | 5294 | 18506 | 18488 | 51.2 | 52.6 | 1.2 | 694 | 75.8 | 41.2 | 40 | 53.2 |
| 1775 | 2795 | 3791 | 3809 | 51.8 | 52.6 | 5295 | 4444 | 4424 | 50.6 | 42.9 | 1.2 | 654 | 75.5 | 40.5 | 40 | 53.1 |
| 1776 | 2796 | 17840 | 17859 | 50.8 | 45 | 5296 | 18233 | 18215 | 51.3 | 52.6 | 0.5 | 394 | 74.9 | 40.9 | 40 | 52.8 |
| 1777 | 2797 | 17840 | 17859 | 50.8 | 45 | 5297 | 18233 | 18214 | 52 | 50 | 1.3 | 394 | 74.9 | 40.9 | 40 | 52.8 |
| 1778 | 2798 | 9930 | 9949 | 52.2 | 50 | 5298 | 10356 | 10336 | 52.4 | 47.6 | 0.2 | 427 | 75.6 | 42.2 | 40 | 53.7 |
| 1779 | 2799 | 15211 | 15230 | 50.2 | 45 | 5299 | 15949 | 15930 | 51.1 | 45 | 0.9 | 739 | 75.5 | 40.3 | 40 | 53 |
| 1780 | 2800 | 98 | 118 | 50.6 | 42.9 | 5300 | 253 | 233 | 51.8 | 47.6 | 1.2 | 156 | 74.5 | 46.2 | 40 | 52.4 |
| 1781 | 2801 | 18004 | 18023 | 51.1 | 50 | 5301 | 18233 | 18214 | 52 | 50 | 0.9 | 230 | 75 | 43.9 | 40 | 52.9 |
| 1782 | 2802 | 24420 | 24440 | 50.8 | 42.9 | 5302 | 24936 | 24919 | 51.8 | 50 | 1 | 517 | 75.9 | 42.2 | 40 | 53.5 |
| 1783 | 2803 | 24420 | 24440 | 50.8 | 42.9 | 5303 | 24938 | 24921 | 50.4 | 50 | 0.4 | 519 | 75.8 | 42 | 40 | 53.3 |
| 1784 | 2804 | 98 | 118 | 50.6 | 42.9 | 5304 | 254 | 235 | 50 | 45 | 0.6 | 157 | 74.4 | 45.9 | 40 | 52.2 |
| 1785 | 2805 | 11540 | 11557 | 50.4 | 50 | 5305 | 11727 | 11707 | 51.1 | 42.9 | 0.7 | 188 | 73.1 | 41 | 40 | 51.4 |
| 1786 | 2806 | 98 | 118 | 50.6 | 42.9 | 5306 | 642 | 622 | 51.6 | 47.6 | 0.9 | 545 | 79.1 | 49.7 | 40 | 55.6 |
| 1787 | 2807 | 15211 | 15230 | 50.2 | 45 | 5307 | 15595 | 15576 | 50.8 | 45 | 0.6 | 385 | 75.1 | 41.3 | 40 | 52.7 |
| 1788 | 2808 | 15255 | 15273 | 50.3 | 52.6 | 5308 | 15767 | 15747 | 50 | 42.9 | 0.3 | 513 | 75.1 | 40.2 | 40 | 52.6 |
| 1789 | 2809 | 12373 | 12391 | 50.8 | 47.4 | 5309 | 12911 | 12891 | 51.2 | 47.6 | 0.4 | 539 | 76.1 | 42.5 | 40 | 53.6 |
| 1790 | 2810 | 18009 | 18028 | 51.6 | 55 | 5310 | 18697 | 18679 | 51.9 | 52.6 | 0.2 | 689 | 76.4 | 42.5 | 40 | 54 |
| 1791 | 2811 | 18009 | 18028 | 51.6 | 55 | 5311 | 18696 | 18673 | 53.4 | 41.7 | 1.8 | 688 | 76.3 | 42.4 | 40 | 54 |
| 1792 | 2812 | 18009 | 18028 | 51.6 | 55 | 5312 | 18239 | 18220 | 50 | 45 | 1.6 | 231 | 74.9 | 43.7 | 40 | 52.5 |
| 1793 | 2813 | 18009 | 18028 | 51.6 | 55 | 5313 | 18238 | 18219 | 50.3 | 45 | 1.4 | 230 | 75 | 43.9 | 40 | 52.7 |
| 1794 | 2814 | 24417 | 24436 | 52.6 | 50 | 5314 | 25079 | 25061 | 52.7 | 52.6 | 0.1 | 663 | 75.8 | 41.3 | 40 | 54 |
| 1795 | 2815 | 10250 | 10271 | 50.6 | 45.5 | 5315 | 10356 | 10336 | 52.4 | 47.6 | 1.8 | 107 | 70.8 | 42.1 | 40 | 49.9 |
| 1796 | 2816 | 1356 | 1375 | 53.8 | 55 | 5316 | 1484 | 1464 | 54.3 | 47.6 | 0.5 | 129 | 74.4 | 48.1 | 40 | 53.3 |
| 1797 | 2817 | 1356 | 1375 | 53.8 | 55 | 5317 | 1484 | 1465 | 53.8 | 50 | 0 | 129 | 74.4 | 48.1 | 40 | 53.3 |
| 1798 | 2818 | 1356 | 1375 | 53.8 | 55 | 5318 | 1484 | 1466 | 53.1 | 52.6 | 0.7 | 129 | 74.4 | 48.1 | 40 | 53.1 |
| 1799 | 2819 | 24418 | 24436 | 50 | 47.4 | 5319 | 24560 | 24542 | 50.2 | 47.4 | 0.1 | 143 | 72.4 | 42 | 40 | 50.8 |
| 1800 | 2820 | 18008 | 18028 | 53 | 52.4 | 5320 | 18696 | 18672 | 53.9 | 40 | 0.9 | 689 | 76.3 | 42.4 | 40 | 54.4 |
| 1801 | 2821 | 25363 | 25381 | 51.1 | 52.6 | 5321 | 25646 | 25627 | 50.5 | 45 | 0.7 | 284 | 74.1 | 40.5 | 40 | 52.1 |
| 1802 | 2822 | 9131 | 9151 | 50.4 | 42.9 | 5322 | 9333 | 9315 | 52.2 | 52.6 | 1.9 | 203 | 74.6 | 43.8 | 40 | 52.4 |
| 1803 | 2823 | 9131 | 9151 | 50.4 | 42.9 | 5323 | 9374 | 9353 | 50.1 | 40.9 | 0.3 | 244 | 74.6 | 42.6 | 40 | 52.4 |
| 1804 | 2824 | 24380 | 24399 | 55 | 55 | 5324 | 24582 | 24560 | 54.2 | 52.2 | 0.9 | 203 | 74.4 | 43.3 | 40 | 53.4 |
| 1805 | 2825 | 19802 | 19820 | 53 | 52.6 | 5325 | 19921 | 19900 | 51.8 | 45.5 | 1.2 | 120 | 72.4 | 44.2 | 40 | 51.3 |
| 1806 | 2826 | 3055 | 3075 | 51.8 | 47.6 | 5326 | 3210 | 3190 | 50.5 | 47.6 | 1.3 | 156 | 74.2 | 45.5 | 40 | 52.2 |
| 1807 | 2827 | 3055 | 3075 | 51.8 | 47.6 | 5327 | 3207 | 3187 | 50.5 | 47.6 | 1.3 | 153 | 74 | 45.1 | 40 | 52 |
| 1808 | 2828 | 3055 | 3076 | 52.4 | 45.5 | 5328 | 3210 | 3190 | 50.5 | 47.6 | 2 | 156 | 74.2 | 45.5 | 40 | 52.2 |
| 1809 | 2829 | 24379 | 24398 | 55 | 55 | 5329 | 24582 | 24560 | 54.2 | 52.2 | 0.9 | 204 | 74.3 | 43.1 | 40 | 53.3 |
| 1810 | 2830 | 7876 | 7895 | 51.5 | 45 | 5330 | 8054 | 8035 | 50.4 | 50 | 1.1 | 179 | 73.5 | 42.5 | 40 | 51.7 |
| 1811 | 2831 | 3055 | 3076 | 52.4 | 45.5 | 5331 | 3207 | 3187 | 50.5 | 47.6 | 2 | 153 | 74 | 45.1 | 40 | 52 |
| 1812 | 2832 | 9130 | 9150 | 51.3 | 42.9 | 5332 | 9324 | 9300 | 52.9 | 40 | 1.6 | 195 | 73.9 | 42.6 | 40 | 52.2 |
| 1813 | 2833 | 9130 | 9150 | 51.3 | 42.9 | 5333 | 9324 | 9301 | 52.4 | 41.7 | 1.1 | 195 | 73.9 | 42.6 | 40 | 52.2 |
| 1814 | 2834 | 8794 | 8813 | 51.6 | 45 | 5334 | 9324 | 9301 | 52.4 | 41.7 | 0.8 | 531 | 75.7 | 41.6 | 40 | 53.6 |
| 1815 | 2835 | 8794 | 8813 | 51.6 | 45 | 5335 | 9324 | 9300 | 52.9 | 40 | 1.3 | 531 | 75.7 | 41.6 | 40 | 53.6 |
| 1816 | 2836 | 9130 | 9150 | 51.3 | 42.9 | 5336 | 9328 | 9310 | 51 | 52.6 | 0.3 | 199 | 74.2 | 43.2 | 40 | 52.4 |
| 1817 | 2837 | 9130 | 9150 | 51.3 | 42.9 | 5337 | 9333 | 9315 | 52.2 | 52.6 | 0.9 | 204 | 74.5 | 43.6 | 40 | 52.6 |
| 1818 | 2838 | 24179 | 24200 | 53.3 | 40.9 | 5338 | 24807 | 24786 | 51.7 | 45.5 | 1.6 | 629 | 75.8 | 41.3 | 40 | 53.7 |
| 1819 | 2839 | 4593 | 4613 | 51.5 | 47.6 | 5339 | 4708 | 4690 | 50.3 | 47.4 | 1.3 | 116 | 71.4 | 42.2 | 40 | 50.2 |
| 1820 | 2840 | 9130 | 9150 | 51.3 | 42.9 | 5340 | 9374 | 9353 | 50.1 | 40.9 | 1.2 | 245 | 74.6 | 42.4 | 40 | 52.3 |
| 1821 | 2841 | 29180 | 29199 | 50.1 | 40 | 5341 | 29412 | 29393 | 50.3 | 45 | 0.2 | 233 | 75.5 | 45.1 | 40 | 53 |
| 1822 | 2842 | 25348 | 25366 | 51.2 | 47.4 | 5342 | 25772 | 25753 | 51.9 | 50 | 0.8 | 425 | 74.9 | 40.5 | 40 | 52.9 |
| 1823 | 2843 | 24179 | 24200 | 53.3 | 40.9 | 5343 | 24815 | 24792 | 53.4 | 41.7 | 0.2 | 637 | 75.8 | 41.3 | 40 | 54.1 |
| 1824 | 2844 | 8794 | 8813 | 51.6 | 45 | 5344 | 9101 | 9081 | 50.5 | 47.6 | 1.2 | 308 | 74.8 | 41.6 | 40 | 52.6 |
| 1825 | 2845 | 16861 | 16880 | 50.8 | 50 | 5345 | 17056 | 17035 | 51.8 | 45.5 | 1 | 196 | 74.7 | 44.4 | 40 | 52.6 |
| 1826 | 2846 | 16562 | 16581 | 52.6 | 50 | 5346 | 17038 | 17021 | 50.7 | 50 | 1.9 | 477 | 75.7 | 41.9 | 40 | 53.3 |
| 1827 | 2847 | 16562 | 16581 | 52.6 | 50 | 5347 | 17039 | 17022 | 51.4 | 50 | 1.2 | 478 | 75.6 | 41.8 | 40 | 53.5 |
| 1828 | 2848 | 16562 | 16581 | 52.6 | 50 | 5348 | 17041 | 17023 | 53.5 | 52.6 | 0.9 | 480 | 75.7 | 41.9 | 40 | 53.8 |
| 1829 | 2849 | 3090 | 3110 | 50.3 | 42.9 | 5349 | 3647 | 3628 | 50.6 | 45 | 0.3 | 558 | 76.2 | 42.7 | 40 | 53.5 |
| 1830 | 2850 | 16562 | 16580 | 51.9 | 52.6 | 5350 | 17038 | 17021 | 50.7 | 50 | 1.2 | 477 | 75.7 | 41.9 | 40 | 53.3 |
| 1831 | 2851 | 16562 | 16580 | 51.9 | 52.6 | 5351 | 17039 | 17022 | 51.4 | 50 | 0.5 | 478 | 75.6 | 41.8 | 40 | 53.5 |
| 1832 | 2852 | 24178 | 24198 | 52.7 | 42.9 | 5352 | 24815 | 24792 | 53.4 | 41.7 | 0.7 | 638 | 75.7 | 41.2 | 40 | 53.9 |
| 1833 | 2853 | 16562 | 16580 | 51.9 | 52.6 | 5353 | 17041 | 17023 | 53.5 | 52.6 | 1.6 | 480 | 75.7 | 41.9 | 40 | 53.6 |
| 1834 | 2854 | 24179 | 24198 | 51 | 45 | 5354 | 24807 | 24786 | 51.7 | 45.5 | 0.7 | 629 | 75.8 | 41.3 | 40 | 53.5 |
| 1835 | 2855 | 24179 | 24198 | 51 | 45 | 5355 | 24818 | 24797 | 51.6 | 40.9 | 0.6 | 640 | 75.8 | 41.2 | 40 | 53.4 |
| 1836 | 2856 | 3090 | 3110 | 50.3 | 42.9 | 5356 | 3646 | 3625 | 52 | 40.9 | 1.7 | 557 | 76.1 | 42.5 | 40 | 53.5 |
| 1837 | 2857 | 3089 | 3110 | 51.8 | 45.5 | 5357 | 3650 | 3631 | 53.1 | 50 | 1.3 | 562 | 76.3 | 42.9 | 40 | 54 |
| 1838 | 2858 | 29259 | 29279 | 54 | 52.4 | 5358 | 29358 | 29339 | 52.8 | 50 | 1.1 | 100 | 72.4 | 47 | 40 | 51.6 |
| 1839 | 2859 | 8794 | 8813 | 51.6 | 45 | 5359 | 8928 | 8911 | 51.9 | 50 | 0.2 | 135 | 72.2 | 42.2 | 40 | 51.1 |
| 1840 | 2860 | 24176 | 24197 | 52.1 | 40.9 | 5360 | 24815 | 24792 | 53.4 | 41.7 | 1.3 | 640 | 75.8 | 41.2 | 40 | 53.8 |
| 1841 | 2861 | 29259 | 29277 | 50.9 | 52.6 | 5361 | 29358 | 29339 | 52.8 | 50 | 2 | 100 | 72.4 | 47 | 40 | 51.1 |
| 1842 | 2862 | 29257 | 29276 | 51.3 | 50 | 5362 | 29358 | 29339 | 52.8 | 50 | 1.5 | 102 | 72.6 | 47.1 | 40 | 51.3 |
| 1843 | 2863 | 9915 | 9935 | 51.8 | 47.6 | 5363 | 10017 | 9999 | 52.8 | 52.6 | 1 | 103 | 72.9 | 47.6 | 40 | 51.6 |
| 1844 | 2864 | 4639 | 4659 | 51.1 | 47.6 | 5364 | 5306 | 5288 | 52.4 | 52.6 | 1.3 | 668 | 75.6 | 40.9 | 40 | 53.4 |
| 1845 | 2865 | 24178 | 24197 | 50.3 | 40 | 5365 | 24807 | 24786 | 51.7 | 45.5 | 1.4 | 630 | 75.8 | 41.3 | 40 | 53.2 |
| 1846 | 2866 | 28653 | 28671 | 50.2 | 52.6 | 5366 | 29414 | 29395 | 50.5 | 50 | 0.3 | 762 | 78 | 46.2 | 40 | 54.7 |
| 1847 | 2867 | 28653 | 28671 | 50.2 | 52.6 | 5367 | 29412 | 29393 | 50.3 | 45 | 0.1 | 760 | 78 | 46.2 | 40 | 54.7 |
| 1848 | 2868 | 28652 | 28671 | 52.8 | 55 | 5368 | 29358 | 29339 | 52.8 | 50 | 0 | 707 | 78 | 46.4 | 40 | 55.5 |
| 1849 | 2869 | 15752 | 15772 | 50.8 | 47.6 | 5369 | 16213 | 16195 | 50.8 | 52.6 | 0 | 462 | 75.4 | 41.3 | 40 | 53.1 |
| 1850 | 2870 | 24178 | 24197 | 50.3 | 40 | 5370 | 24818 | 24797 | 51.6 | 40.9 | 1.4 | 641 | 75.7 | 41.2 | 40 | 53.2 |
| 1851 | 2871 | 19794 | 19814 | 51.7 | 47.6 | 5371 | 19909 | 19885 | 52.5 | 40 | 0.8 | 116 | 71.8 | 43.1 | 40 | 50.8 |
| 1852 | 2872 | 8866 | 8885 | 51.1 | 45 | 5372 | 9341 | 9322 | 51.1 | 50 | 0 | 476 | 75.6 | 41.8 | 40 | 53.4 |
| 1853 | 2873 | 15951 | 15973 | 52.1 | 43.5 | 5373 | 16175 | 16155 | 51.8 | 47.6 | 0.3 | 225 | 73.7 | 40.9 | 40 | 52.2 |
| 1854 | 2874 | 24174 | 24195 | 52.5 | 40.9 | 5374 | 24815 | 24791 | 54.5 | 40 | 2 | 642 | 75.8 | 41.3 | 40 | 53.9 |
| 1855 | 2875 | 8866 | 8885 | 51.1 | 45 | 5375 | 9340 | 9319 | 50.8 | 45.5 | 0.3 | 475 | 75.6 | 41.7 | 40 | 53.2 |
| 1856 | 2876 | 15951 | 15973 | 52.1 | 43.5 | 5376 | 16169 | 16151 | 51.3 | 52.6 | 0.8 | 219 | 73.5 | 40.6 | 40 | 51.9 |
| 1857 | 2877 | 15951 | 15974 | 53.3 | 41.7 | 5377 | 16175 | 16154 | 53.4 | 45.5 | 0.1 | 225 | 73.7 | 40.9 | 40 | 52.7 |
| 1858 | 2878 | 27437 | 27456 | 50.2 | 40 | 5378 | 27541 | 27521 | 51.7 | 47.6 | 1.5 | 105 | 71.8 | 44.8 | 40 | 50.4 |
| 1859 | 2879 | 15650 | 15674 | 52.9 | 40 | 5379 | 16210 | 16192 | 54.3 | 52.6 | 1.4 | 561 | 75.1 | 40.1 | 40 | 53.6 |
| 1860 | 2880 | 8866 | 8885 | 51.1 | 45 | 5380 | 9334 | 9316 | 51.3 | 52.6 | 0.2 | 469 | 75.7 | 42 | 40 | 53.4 |
| 1861 | 2881 | 8866 | 8885 | 51.1 | 45 | 5381 | 9310 | 9291 | 51.2 | 45 | 0.1 | 445 | 75.3 | 41.3 | 40 | 53.2 |
| 1862 | 2882 | 8866 | 8885 | 51.1 | 45 | 5382 | 9252 | 9234 | 51.4 | 52.6 | 0.3 | 387 | 75.1 | 41.3 | 40 | 53 |
| 1863 | 2883 | 3360 | 3379 | 50.7 | 45 | 5383 | 3494 | 3473 | 50.4 | 40.9 | 0.3 | 135 | 73.7 | 45.9 | 40 | 51.8 |
| 1864 | 2884 | 8866 | 8885 | 51.1 | 45 | 5384 | 9248 | 9229 | 50.1 | 45 | 1 | 383 | 75.1 | 41.3 | 40 | 52.7 |
| 1865 | 2885 | 18081 | 18099 | 51.2 | 52.6 | 5385 | 18697 | 18679 | 51.9 | 52.6 | 0.7 | 617 | 76.3 | 42.6 | 40 | 53.9 |
| 1866 | 2886 | 8865 | 8884 | 50.4 | 45 | 5386 | 9257 | 9238 | 50.5 | 45 | 0.1 | 393 | 75 | 41 | 40 | 52.7 |
| 1867 | 2887 | 18081 | 18099 | 51.2 | 52.6 | 5387 | 18239 | 18220 | 50 | 45 | 1.2 | 159 | 74 | 44.7 | 40 | 51.9 |
| 1868 | 2888 | 18081 | 18099 | 51.2 | 52.6 | 5388 | 18238 | 18219 | 50.3 | 45 | 0.9 | 158 | 74.1 | 44.9 | 40 | 52 |
| 1869 | 2889 | 28117 | 28135 | 50.6 | 52.6 | 5389 | 28505 | 28487 | 50.2 | 47.4 | 0.4 | 389 | 79.5 | 51.9 | 40 | 55.8 |
| 1870 | 2890 | 8866 | 8885 | 51.1 | 45 | 5390 | 9109 | 9087 | 50.5 | 43.5 | 0.6 | 244 | 73.9 | 41 | 40 | 52 |
| 1871 | 2891 | 9055 | 9079 | 52.8 | 40 | 5391 | 9724 | 9706 | 51.3 | 52.6 | 1.5 | 670 | 75.4 | 40.3 | 40 | 53.3 |
| 1872 | 2892 | 3403 | 3423 | 54.1 | 47.6 | 5392 | 3502 | 3478 | 55.8 | 48 | 1.7 | 100 | 71.6 | 45 | 40 | 51.5 |
| 1873 | 2893 | 28855 | 28874 | 52.9 | 50 | 5393 | 29306 | 29288 | 53.5 | 52.6 | 0.6 | 452 | 77.1 | 45.6 | 40 | 54.9 |
| 1874 | 2894 | 24173 | 24194 | 52.5 | 40.9 | 5394 | 24815 | 24792 | 53.4 | 41.7 | 0.9 | 643 | 75.8 | 41.2 | 40 | 53.9 |
| 1875 | 2895 | 3094 | 3113 | 50 | 50 | 5395 | 3647 | 3628 | 50.6 | 45 | 0.6 | 554 | 76.2 | 42.8 | 40 | 53.5 |
| 1876 | 2896 | 24174 | 24194 | 50.9 | 42.9 | 5396 | 24807 | 24786 | 51.7 | 45.5 | 0.8 | 634 | 75.8 | 41.3 | 40 | 53.4 |
| 1877 | 2897 | 28856 | 28875 | 52.2 | 50 | 5397 | 29306 | 29288 | 53.5 | 52.6 | 1.3 | 451 | 77.1 | 45.7 | 40 | 54.7 |
| 1878 | 2898 | 24174 | 24194 | 50.9 | 42.9 | 5398 | 24818 | 24797 | 51.6 | 40.9 | 0.7 | 645 | 75.8 | 41.2 | 40 | 53.4 |
| 1879 | 2899 | 28857 | 28876 | 51.7 | 45 | 5399 | 29306 | 29288 | 53.5 | 52.6 | 1.8 | 450 | 77.1 | 45.6 | 40 | 54.6 |
| 1880 | 2900 | 8858 | 8877 | 51.2 | 45 | 5400 | 9254 | 9236 | 50.6 | 47.4 | 0.6 | 397 | 75 | 41.1 | 40 | 52.8 |
| 1881 | 2901 | 16553 | 16571 | 53.4 | 52.6 | 5401 | 16777 | 16758 | 51.5 | 50 | 1.9 | 225 | 73.7 | 40.9 | 40 | 52.1 |
| 1882 | 2902 | 29197 | 29219 | 54.8 | 47.8 | 5402 | 29301 | 29282 | 55.3 | 55 | 0.5 | 105 | 73.4 | 48.6 | 40 | 52.9 |
| 1883 | 2903 | 29198 | 29219 | 52.6 | 45.5 | 5403 | 29306 | 29288 | 53.5 | 52.6 | 0.9 | 109 | 73.3 | 47.7 | 40 | 52.2 |
| 1884 | 2904 | 28857 | 28877 | 52.3 | 42.9 | 5404 | 29306 | 29288 | 53.5 | 52.6 | 1.2 | 450 | 77.1 | 45.6 | 40 | 54.8 |
| 1885 | 2905 | 29199 | 29219 | 51.2 | 42.9 | 5405 | 29298 | 29280 | 51.4 | 52.6 | 0.2 | 100 | 72.4 | 47 | 40 | 51.1 |
| 1886 | 2906 | 3094 | 3113 | 50 | 50 | 5406 | 3646 | 3625 | 52 | 40.9 | 2 | 553 | 76.2 | 42.7 | 40 | 53.4 |
| 1887 | 2907 | 3224 | 3243 | 52.3 | 50 | 5407 | 3650 | 3631 | 53.1 | 50 | 0.8 | 427 | 75.5 | 41.9 | 40 | 53.7 |
| 1888 | 2908 | 29195 | 29216 | 53.8 | 45.5 | 5408 | 29306 | 29287 | 54.6 | 55 | 0.8 | 112 | 73.6 | 48.2 | 40 | 52.8 |
| 1889 | 2909 | 28867 | 28885 | 51.5 | 52.6 | 5409 | 29358 | 29339 | 52.8 | 50 | 1.4 | 492 | 76.9 | 44.9 | 40 | 54.4 |
| 1890 | 2910 | 29196 | 29216 | 52.5 | 47.6 | 5410 | 29298 | 29279 | 52.6 | 55 | 0.1 | 103 | 73.3 | 48.5 | 40 | 52.1 |
| 1891 | 2911 | 28867 | 28886 | 53.2 | 50 | 5411 | 29415 | 29395 | 53.4 | 52.4 | 0.2 | 549 | 77.1 | 45 | 40 | 55 |
| 1892 | 2912 | 3093 | 3113 | 51.7 | 47.6 | 5412 | 3650 | 3631 | 53.1 | 50 | 1.4 | 558 | 76.3 | 42.8 | 40 | 54 |
| 1893 | 2913 | 3225 | 3243 | 50.9 | 52.6 | 5413 | 3646 | 3625 | 52 | 40.9 | 1.2 | 422 | 75.4 | 41.7 | 40 | 53.1 |
| 1894 | 2914 | 3225 | 3243 | 50.9 | 52.6 | 5414 | 3647 | 3628 | 50.6 | 45 | 0.3 | 423 | 75.5 | 41.8 | 40 | 53.1 |
| 1895 | 2915 | 28867 | 28886 | 53.2 | 50 | 5415 | 29306 | 29287 | 54.6 | 55 | 1.4 | 440 | 76.9 | 45.2 | 40 | 54.9 |
| 1896 | 2916 | 3223 | 3241 | 50.2 | 52.6 | 5416 | 3500 | 3481 | 51.2 | 50 | 1 | 278 | 74.7 | 42.1 | 40 | 52.5 |
| 1897 | 2917 | 28867 | 28886 | 53.2 | 50 | 5417 | 29298 | 29279 | 52.6 | 55 | 0.5 | 432 | 76.8 | 45.1 | 40 | 54.7 |
| 1898 | 2918 | 24034 | 24053 | 53.4 | 55 | 5418 | 24815 | 24791 | 54.5 | 40 | 1.1 | 782 | 76.3 | 42.1 | 40 | 54.5 |
| 1899 | 2919 | 3221 | 3239 | 51.5 | 52.6 | 5419 | 3650 | 3631 | 53.1 | 50 | 1.6 | 430 | 75.5 | 41.9 | 40 | 53.4 |
| 1900 | 2920 | 18080 | 18099 | 53 | 50 | 5420 | 18696 | 18673 | 53.4 | 41.7 | 0.5 | 617 | 76.2 | 42.5 | 40 | 54.3 |
| 1901 | 2921 | 3095 | 3116 | 51.9 | 45.5 | 5421 | 3650 | 3631 | 53.1 | 50 | 1.2 | 556 | 76.2 | 42.8 | 40 | 54 |
| 1902 | 2922 | 18080 | 18099 | 53 | 50 | 5422 | 18696 | 18672 | 53.9 | 40 | 1 | 617 | 76.2 | 42.5 | 40 | 54.3 |
| 1903 | 2923 | 28868 | 28887 | 50.7 | 45 | 5423 | 29298 | 29279 | 52.6 | 55 | 1.9 | 431 | 76.8 | 45 | 40 | 54.1 |
| 1904 | 2924 | 3218 | 3238 | 52.1 | 47.6 | 5424 | 3650 | 3631 | 53.1 | 50 | 1 | 433 | 75.5 | 41.8 | 40 | 53.6 |
| 1905 | 2925 | 8867 | 8886 | 50.7 | 50 | 5425 | 9252 | 9234 | 51.4 | 52.6 | 0.8 | 386 | 75.1 | 41.5 | 40 | 52.9 |
| 1906 | 2926 | 28867 | 28887 | 53.7 | 47.6 | 5426 | 29306 | 29287 | 54.6 | 55 | 0.8 | 440 | 76.9 | 45.2 | 40 | 55.1 |
| 1907 | 2927 | 3218 | 3237 | 50.5 | 45 | 5427 | 3497 | 3478 | 51.3 | 50 | 0.8 | 280 | 74.6 | 41.8 | 40 | 52.5 |
| 1908 | 2928 | 29195 | 29215 | 53.2 | 47.6 | 5428 | 29306 | 29287 | 54.6 | 55 | 1.4 | 112 | 73.6 | 48.2 | 40 | 52.6 |
| 1909 | 2929 | 29196 | 29215 | 51.8 | 50 | 5429 | 29298 | 29279 | 52.6 | 55 | 0.8 | 103 | 73.3 | 48.5 | 40 | 51.9 |
| 1910 | 2930 | 3218 | 3237 | 50.5 | 45 | 5430 | 3500 | 3481 | 51.2 | 50 | 0.6 | 283 | 74.6 | 41.7 | 40 | 52.5 |
| 1911 | 2931 | 8867 | 8886 | 50.7 | 50 | 5431 | 9245 | 9226 | 50 | 45 | 0.6 | 379 | 75 | 41.2 | 40 | 52.6 |
| 1912 | 2932 | 28868 | 28888 | 51.4 | 42.9 | 5432 | 29298 | 29279 | 52.6 | 55 | 1.2 | 431 | 76.8 | 45 | 40 | 54.3 |
| 1913 | 2933 | 8867 | 8886 | 50.7 | 50 | 5433 | 9107 | 9086 | 51.6 | 45.5 | 0.9 | 241 | 74.1 | 41.5 | 40 | 52.2 |
| 1914 | 2934 | 28867 | 28888 | 54.3 | 45.5 | 5434 | 29306 | 29287 | 54.6 | 55 | 0.3 | 440 | 76.9 | 45.2 | 40 | 55.2 |
| 1915 | 2935 | 19906 | 19925 | 50.1 | 50 | 5435 | 20615 | 20597 | 50.6 | 47.4 | 0.5 | 710 | 75.5 | 40.3 | 40 | 53 |
| 1916 | 2936 | 16551 | 16568 | 51.1 | 50 | 5436 | 16775 | 16756 | 50.3 | 45 | 0.8 | 225 | 73.8 | 41.3 | 40 | 51.9 |
| 1917 | 2937 | 8861 | 8880 | 50.2 | 45 | 5437 | 9341 | 9322 | 51.1 | 50 | 0.9 | 481 | 75.5 | 41.6 | 40 | 53 |
| 1918 | 2938 | 16368 | 16387 | 50.2 | 45 | 5438 | 16781 | 16761 | 51.3 | 47.6 | 1 | 414 | 75 | 40.8 | 40 | 52.7 |
| 1919 | 2939 | 3055 | 3074 | 51.1 | 50 | 5439 | 3209 | 3189 | 50.5 | 47.6 | 0.6 | 155 | 74.1 | 45.2 | 40 | 52.1 |
| 1920 | 2940 | 3217 | 3236 | 51.1 | 50 | 5440 | 3650 | 3631 | 53.1 | 50 | 2 | 434 | 75.5 | 41.9 | 40 | 53.3 |
| 1921 | 2941 | 28868 | 28889 | 52 | 40.9 | 5441 | 29298 | 29279 | 52.6 | 55 | 0.6 | 431 | 76.8 | 45 | 40 | 54.5 |
| 1922 | 2942 | 28867 | 28889 | 54.8 | 43.5 | 5442 | 29306 | 29287 | 54.6 | 55 | 0.2 | 440 | 76.9 | 45.2 | 40 | 55.3 |
| 1923 | 2943 | 3404 | 3422 | 50.5 | 47.4 | 5443 | 3503 | 3484 | 51.5 | 50 | 0.9 | 100 | 71.6 | 45 | 40 | 50.4 |
| 1924 | 2944 | 16368 | 16387 | 50.2 | 45 | 5444 | 16777 | 16758 | 51.5 | 50 | 1.2 | 410 | 75 | 40.7 | 40 | 52.6 |
| 1925 | 2945 | 24029 | 24047 | 52.1 | 52.6 | 5445 | 24815 | 24792 | 53.4 | 41.7 | 1.3 | 787 | 76.3 | 42.1 | 40 | 54.1 |
| 1926 | 2946 | 16368 | 16387 | 50.2 | 45 | 5446 | 16711 | 16691 | 51 | 42.9 | 0.8 | 344 | 75.1 | 41.9 | 40 | 52.7 |
| 1927 | 2947 | 28867 | 28890 | 55.2 | 41.7 | 5447 | 29306 | 29287 | 54.6 | 55 | 0.6 | 440 | 76.9 | 45.2 | 40 | 55.3 |
| 1928 | 2948 | 29196 | 29214 | 51.1 | 52.6 | 5448 | 29298 | 29279 | 52.6 | 55 | 1.5 | 103 | 73.3 | 48.5 | 40 | 51.7 |
| 1929 | 2949 | 18488 | 18507 | 53.7 | 55 | 5449 | 19224 | 19200 | 52.4 | 40 | 1.3 | 737 | 76 | 41.5 | 40 | 54 |
| 1930 | 2950 | 28395 | 28413 | 50.2 | 42.1 | 5450 | 28506 | 28488 | 50.2 | 47.4 | 0 | 112 | 74.4 | 50 | 40 | 52.2 |
| 1931 | 2951 | 16551 | 16568 | 51.1 | 50 | 5451 | 17032 | 17011 | 52 | 45.5 | 0.9 | 482 | 75.8 | 42.1 | 40 | 53.5 |
| 1932 | 2952 | 28871 | 28891 | 50.9 | 42.9 | 5452 | 29358 | 29339 | 52.8 | 50 | 1.9 | 488 | 76.9 | 44.9 | 40 | 54.2 |
| 1933 | 2953 | 28871 | 28891 | 50.9 | 42.9 | 5453 | 29298 | 29280 | 51.4 | 52.6 | 0.5 | 428 | 76.8 | 45.1 | 40 | 54.2 |
| 1934 | 2954 | 28870 | 28891 | 52.2 | 40.9 | 5454 | 29306 | 29288 | 53.5 | 52.6 | 1.2 | 437 | 76.8 | 45.1 | 40 | 54.6 |
| 1935 | 2955 | 28868 | 28891 | 53.8 | 41.7 | 5455 | 29301 | 29282 | 55.3 | 55 | 1.5 | 434 | 76.9 | 45.2 | 40 | 55.1 |
| 1936 | 2956 | 3404 | 3422 | 50.5 | 47.4 | 5456 | 3504 | 3485 | 50.4 | 45 | 0.1 | 101 | 71.5 | 44.6 | 40 | 50.3 |
| 1937 | 2957 | 29195 | 29213 | 51.9 | 52.6 | 5457 | 29298 | 29279 | 52.6 | 55 | 0.7 | 104 | 73.1 | 48.1 | 40 | 51.9 |
| 1938 | 2958 | 28938 | 28956 | 50.8 | 47.4 | 5458 | 29298 | 29280 | 51.4 | 52.6 | 0.6 | 361 | 76.4 | 44.9 | 40 | 53.8 |
| 1939 | 2959 | 18488 | 18507 | 53.7 | 55 | 5459 | 19210 | 19191 | 52 | 50 | 1.7 | 723 | 76 | 41.6 | 40 | 53.9 |
| 1940 | 2960 | 3095 | 3116 | 51.9 | 45.5 | 5460 | 3647 | 3628 | 50.6 | 45 | 1.3 | 553 | 76.2 | 42.7 | 40 | 53.6 |
| 1941 | 2961 | 3214 | 3233 | 51.1 | 50 | 5461 | 3497 | 3478 | 51.3 | 50 | 0.2 | 284 | 74.7 | 41.9 | 40 | 52.7 |
| 1942 | 2962 | 24017 | 24039 | 53 | 43.5 | 5462 | 24815 | 24791 | 54.5 | 40 | 1.5 | 799 | 76.2 | 41.9 | 40 | 54.4 |
| 1943 | 2963 | 3095 | 3116 | 51.9 | 45.5 | 5463 | 3646 | 3625 | 52 | 40.9 | 0.1 | 552 | 76.1 | 42.6 | 40 | 54 |
| 1944 | 2964 | 18550 | 18571 | 50.4 | 40.9 | 5464 | 19215 | 19194 | 50.2 | 40.9 | 0.2 | 666 | 75.8 | 41.1 | 40 | 53.2 |
| 1945 | 2965 | 3214 | 3233 | 51.1 | 50 | 5465 | 3500 | 3481 | 51.2 | 50 | 0.1 | 287 | 74.7 | 41.8 | 40 | 52.7 |
| 1946 | 2966 | 18586 | 18603 | 50.4 | 44.4 | 5466 | 19224 | 19200 | 52.4 | 40 | 1.9 | 639 | 75.6 | 40.8 | 40 | 53.1 |
| 1947 | 2967 | 18586 | 18603 | 50.4 | 44.4 | 5467 | 19217 | 19196 | 50.2 | 40.9 | 0.2 | 632 | 75.6 | 41 | 40 | 53.1 |
| 1948 | 2968 | 18586 | 18603 | 50.4 | 44.4 | 5468 | 19215 | 19194 | 50.2 | 40.9 | 0.2 | 630 | 75.6 | 41 | 40 | 53.1 |
| 1949 | 2969 | 18590 | 18608 | 50.6 | 42.1 | 5469 | 19224 | 19200 | 52.4 | 40 | 1.8 | 635 | 75.6 | 40.9 | 40 | 53.2 |
| 1950 | 2970 | 15255 | 15273 | 50.3 | 52.6 | 5470 | 15767 | 15746 | 50.7 | 40.9 | 0.4 | 513 | 75.1 | 40.2 | 40 | 52.7 |
| 1951 | 2971 | 28942 | 28961 | 50.2 | 45 | 5471 | 29414 | 29395 | 50.5 | 50 | 0.3 | 473 | 76.8 | 44.6 | 40 | 53.9 |
| 1952 | 2972 | 18590 | 18608 | 50.6 | 42.1 | 5472 | 19217 | 19196 | 50.2 | 40.9 | 0.3 | 628 | 75.7 | 41.1 | 40 | 53.1 |
| 1953 | 2973 | 3055 | 3074 | 51.1 | 50 | 5473 | 3207 | 3187 | 50.5 | 47.6 | 0.6 | 153 | 74 | 45.1 | 40 | 52 |
| 1954 | 2974 | 18590 | 18608 | 50.6 | 42.1 | 5474 | 19215 | 19194 | 50.2 | 40.9 | 0.3 | 626 | 75.7 | 41.1 | 40 | 53.1 |
| 1955 | 2975 | 18591 | 18611 | 51.7 | 42.9 | 5475 | 19224 | 19200 | 52.4 | 40 | 0.7 | 634 | 75.7 | 41 | 40 | 53.6 |
| 1956 | 2976 | 18591 | 18611 | 51.7 | 42.9 | 5476 | 19217 | 19196 | 50.2 | 40.9 | 1.4 | 627 | 75.7 | 41.1 | 40 | 53.2 |
| 1957 | 2977 | 18591 | 18611 | 51.7 | 42.9 | 5477 | 19215 | 19194 | 50.2 | 40.9 | 1.4 | 625 | 75.7 | 41.1 | 40 | 53.2 |
| 1958 | 2978 | 28546 | 28565 | 52.2 | 50 | 5478 | 28672 | 28654 | 50.6 | 52.6 | 1.6 | 127 | 76.5 | 53.5 | 40 | 53.8 |
| 1959 | 2979 | 29191 | 29210 | 54.4 | 55 | 5479 | 29415 | 29395 | 53.4 | 52.4 | 1 | 225 | 75.7 | 45.8 | 40 | 54.1 |
| 1960 | 2980 | 7880 | 7900 | 50.3 | 42.9 | 5480 | 8190 | 8172 | 50.3 | 47.4 | 0 | 311 | 74.9 | 41.8 | 40 | 52.6 |
| 1961 | 2981 | 3167 | 3189 | 51.6 | 43.5 | 5481 | 3650 | 3631 | 53.1 | 50 | 1.5 | 484 | 75.8 | 42.1 | 40 | 53.6 |
| 1962 | 2982 | 28965 | 28984 | 52.9 | 55 | 5482 | 29306 | 29288 | 53.5 | 52.6 | 0.6 | 342 | 76.5 | 45.3 | 40 | 54.5 |
| 1963 | 2983 | 3166 | 3188 | 51.6 | 43.5 | 5483 | 3650 | 3631 | 53.1 | 50 | 1.5 | 485 | 75.8 | 42.3 | 40 | 53.7 |
| 1964 | 2984 | 8867 | 8887 | 52.3 | 47.6 | 5484 | 9101 | 9081 | 50.5 | 47.6 | 1.9 | 235 | 74.1 | 41.7 | 40 | 52.1 |
| 1965 | 2985 | 23843 | 23863 | 50.3 | 42.9 | 5485 | 24013 | 23995 | 50.3 | 47.4 | 0 | 171 | 73.7 | 43.3 | 40 | 51.8 |
| 1966 | 2986 | 3403 | 3421 | 53.1 | 52.6 | 5486 | 3503 | 3484 | 51.5 | 50 | 1.7 | 101 | 71.9 | 45.5 | 40 | 50.9 |
| 1967 | 2987 | 16549 | 16567 | 54.9 | 52.6 | 5487 | 16777 | 16756 | 53.4 | 45.5 | 1.5 | 229 | 74 | 41.5 | 40 | 52.9 |
| 1968 | 2988 | 8868 | 8889 | 50.4 | 40.9 | 5488 | 9109 | 9087 | 50.5 | 43.5 | 0.1 | 242 | 73.9 | 40.9 | 40 | 51.9 |
| 1969 | 2989 | 8861 | 8880 | 50.2 | 45 | 5489 | 9311 | 9292 | 50.7 | 50 | 0.6 | 451 | 75.3 | 41.2 | 40 | 52.9 |
| 1970 | 2990 | 8868 | 8889 | 50.4 | 40.9 | 5490 | 9257 | 9238 | 50.5 | 45 | 0.1 | 390 | 75 | 41 | 40 | 52.7 |
| 1971 | 2991 | 8868 | 8889 | 50.4 | 40.9 | 5491 | 9313 | 9294 | 50.4 | 50 | 0 | 446 | 75.4 | 41.5 | 40 | 53 |
| 1972 | 2992 | 23841 | 23859 | 50.5 | 52.6 | 5492 | 24013 | 23995 | 50.3 | 47.4 | 0.1 | 173 | 74 | 43.9 | 40 | 52 |
| 1973 | 2993 | 28548 | 28568 | 50.5 | 42.9 | 5493 | 28672 | 28654 | 50.6 | 52.6 | 0 | 125 | 76.2 | 52.8 | 40 | 53.6 |
| 1974 | 2994 | 8867 | 8888 | 52.7 | 45.5 | 5494 | 9310 | 9291 | 51.2 | 45 | 1.5 | 444 | 75.4 | 41.4 | 40 | 53.2 |
| 1975 | 2995 | 28968 | 28988 | 50.9 | 47.6 | 5495 | 29298 | 29279 | 52.6 | 55 | 1.8 | 331 | 76.2 | 44.7 | 40 | 53.7 |
| 1976 | 2996 | 19907 | 19926 | 52.1 | 55 | 5496 | 20615 | 20597 | 50.6 | 47.4 | 1.6 | 709 | 75.5 | 40.3 | 40 | 53.1 |
| 1977 | 2997 | 8861 | 8880 | 50.2 | 45 | 5497 | 9252 | 9235 | 50.1 | 50 | 0.1 | 392 | 75 | 41.1 | 40 | 52.6 |
| 1978 | 2998 | 19909 | 19929 | 50.7 | 52.4 | 5498 | 20615 | 20597 | 50.6 | 47.4 | 0.2 | 707 | 75.5 | 40.3 | 40 | 53.1 |
| 1979 | 2999 | 3361 | 3382 | 51.9 | 45.5 | 5499 | 3500 | 3481 | 51.2 | 50 | 0.7 | 140 | 74.1 | 46.4 | 40 | 52.3 |
| 1980 | 3000 | 18696 | 18715 | 51.7 | 50 | 5500 | 18881 | 18862 | 50.2 | 45 | 1.5 | 186 | 74.1 | 43.5 | 40 | 52.1 |
| 1981 | 3001 | 28968 | 28989 | 51.5 | 45.5 | 5501 | 29298 | 29279 | 52.6 | 55 | 1.1 | 331 | 76.2 | 44.7 | 40 | 53.9 |
| 1982 | 3002 | 19709 | 19730 | 51.3 | 40.9 | 5502 | 19923 | 19903 | 50.9 | 47.6 | 0.4 | 215 | 73.9 | 41.9 | 40 | 52.1 |
| 1983 | 3003 | 3361 | 3382 | 51.9 | 45.5 | 5503 | 3497 | 3478 | 51.3 | 50 | 0.6 | 137 | 74.1 | 46.7 | 40 | 52.4 |
| 1984 | 3004 | 3361 | 3384 | 53.7 | 41.7 | 5504 | 3495 | 3473 | 51.8 | 43.5 | 1.9 | 135 | 74 | 46.7 | 40 | 52.5 |
| 1985 | 3005 | 19709 | 19730 | 51.3 | 40.9 | 5505 | 19924 | 19905 | 50.1 | 50 | 1.2 | 216 | 73.9 | 41.7 | 40 | 51.8 |
| 1986 | 3006 | 16378 | 16397 | 50.4 | 45 | 5506 | 16711 | 16691 | 51 | 42.9 | 0.6 | 334 | 75.2 | 42.2 | 40 | 52.9 |
| 1987 | 3007 | 3361 | 3382 | 51.9 | 45.5 | 5507 | 3504 | 3485 | 50.4 | 45 | 1.5 | 144 | 74.3 | 46.5 | 40 | 52.2 |
| 1988 | 3008 | 18704 | 18724 | 50.8 | 47.6 | 5508 | 19406 | 19388 | 50.6 | 47.4 | 0.1 | 703 | 75.4 | 40.3 | 40 | 53.1 |
| 1989 | 3009 | 8868 | 8889 | 50.4 | 40.9 | 5509 | 9314 | 9295 | 51.1 | 50 | 0.7 | 447 | 75.5 | 41.6 | 40 | 53 |
| 1990 | 3010 | 3361 | 3382 | 51.9 | 45.5 | 5510 | 3503 | 3484 | 51.5 | 50 | 0.5 | 143 | 74.4 | 46.9 | 40 | 52.6 |
| 1991 | 3011 | 19709 | 19730 | 51.3 | 40.9 | 5511 | 19931 | 19912 | 50.9 | 55 | 0.4 | 223 | 74.2 | 42.2 | 40 | 52.3 |
| 1992 | 3012 | 16548 | 16566 | 54.9 | 52.6 | 5512 | 16777 | 16756 | 53.4 | 45.5 | 1.5 | 230 | 73.9 | 41.3 | 40 | 52.9 |
| 1993 | 3013 | 8868 | 8889 | 50.4 | 40.9 | 5513 | 9315 | 9296 | 50 | 45 | 0.4 | 448 | 75.4 | 41.5 | 40 | 52.9 |
| 1994 | 3014 | 22321 | 22341 | 51.6 | 42.9 | 5514 | 22460 | 22441 | 50.7 | 45 | 0.9 | 140 | 71.5 | 40 | 40 | 50.3 |
| 1995 | 3015 | 29182 | 29202 | 51.2 | 42.9 | 5515 | 29412 | 29393 | 50.3 | 45 | 0.9 | 231 | 75.4 | 45 | 40 | 53 |
| 1996 | 3016 | 22173 | 22193 | 51 | 42.9 | 5516 | 22460 | 22441 | 50.7 | 45 | 0.3 | 288 | 74.1 | 40.3 | 40 | 52.1 |
| 1997 | 3017 | 29181 | 29201 | 52.4 | 47.6 | 5517 | 29413 | 29393 | 51.1 | 42.9 | 1.3 | 233 | 75.5 | 45.1 | 40 | 53.3 |
| 1998 | 3018 | 18704 | 18724 | 50.8 | 47.6 | 5518 | 18881 | 18862 | 50.2 | 45 | 0.5 | 178 | 73.8 | 43.3 | 40 | 51.9 |
| 1999 | 3019 | 20751 | 20771 | 51.3 | 47.6 | 5519 | 21301 | 21278 | 51.3 | 41.7 | 0 | 551 | 75.5 | 41 | 40 | 53.3 |
| 2000 | 3020 | 29181 | 29200 | 50 | 45 | 5520 | 29412 | 29393 | 50.3 | 45 | 0.3 | 232 | 75.5 | 45.3 | 40 | 53 |
| 2001 | 3021 | 20751 | 20771 | 51.3 | 47.6 | 5521 | 21304 | 21283 | 50.5 | 40.9 | 0.8 | 554 | 75.5 | 41 | 40 | 53.1 |
| 2002 | 3022 | 29173 | 29197 | 54.2 | 40 | 5522 | 29415 | 29395 | 53.4 | 52.4 | 0.8 | 243 | 75.7 | 45.3 | 40 | 54.1 |
| 2003 | 3023 | 8867 | 8888 | 52.7 | 45.5 | 5523 | 9247 | 9226 | 52 | 45.5 | 0.7 | 381 | 75 | 41.2 | 40 | 53.2 |
| 2004 | 3024 | 8867 | 8888 | 52.7 | 45.5 | 5524 | 9255 | 9236 | 51.1 | 45 | 1.6 | 389 | 75 | 41.1 | 40 | 52.9 |
| 2005 | 3025 | 29178 | 29198 | 51.4 | 42.9 | 5525 | 29412 | 29393 | 50.3 | 45 | 1.1 | 235 | 75.5 | 45.1 | 40 | 53.1 |
| 2006 | 3026 | 3163 | 3185 | 53.6 | 47.8 | 5526 | 3650 | 3631 | 53.1 | 50 | 0.5 | 488 | 75.9 | 42.4 | 40 | 54.2 |
| 2007 | 3027 | 19800 | 19817 | 50.4 | 50 | 5527 | 20033 | 20016 | 50.4 | 50 | 0 | 234 | 74.9 | 43.6 | 41 | 52.7 |
| 2008 | 3028 | 8867 | 8886 | 50.7 | 50 | 5528 | 9376 | 9355 | 51 | 40.9 | 0.3 | 510 | 75.7 | 41.8 | 41 | 53.3 |
| 2009 | 3029 | 19800 | 19817 | 50.4 | 50 | 5529 | 19930 | 19910 | 50.6 | 47.6 | 0.2 | 131 | 72.6 | 43.5 | 41 | 51 |
| 2010 | 3030 | 24418 | 24439 | 52.9 | 45.5 | 5530 | 25082 | 25064 | 51.1 | 52.6 | 1.8 | 665 | 75.8 | 41.2 | 41 | 53.5 |
| 2011 | 3031 | 25771 | 25790 | 51.1 | 45 | 5531 | 26182 | 26161 | 51.2 | 40.9 | 0.1 | 412 | 74.8 | 40.3 | 41 | 52.8 |
| 2012 | 3032 | 12976 | 12994 | 50.3 | 47.4 | 5532 | 13326 | 13306 | 50.7 | 42.9 | 0.3 | 351 | 76.1 | 44.2 | 41 | 53.5 |
| 2013 | 3033 | 12976 | 12994 | 50.3 | 47.4 | 5533 | 13328 | 13307 | 51.2 | 45.5 | 0.9 | 353 | 76.1 | 44.2 | 41 | 53.5 |
| 2014 | 3034 | 2823 | 2844 | 50.4 | 45.5 | 5534 | 3500 | 3481 | 51.2 | 50 | 0.7 | 678 | 76.3 | 42.5 | 41 | 53.7 |
| 2015 | 3035 | 18009 | 18028 | 51.6 | 55 | 5535 | 18223 | 18205 | 53.3 | 52.6 | 1.7 | 215 | 74.5 | 43.3 | 41 | 52.7 |
| 2016 | 3036 | 8223 | 8240 | 50.4 | 50 | 5536 | 8933 | 8916 | 52.2 | 50 | 1.8 | 711 | 75.4 | 40.1 | 41 | 53 |
| 2017 | 3037 | 29180 | 29199 | 50.1 | 40 | 5537 | 29414 | 29395 | 50.5 | 50 | 0.4 | 235 | 75.5 | 45.1 | 41 | 53 |
| 2018 | 3038 | 19800 | 19817 | 50.4 | 50 | 5538 | 19925 | 19906 | 50.1 | 50 | 0.4 | 126 | 72.4 | 43.7 | 41 | 50.8 |
| 2019 | 3039 | 25772 | 25793 | 52.4 | 40.9 | 5539 | 26183 | 26162 | 52.8 | 45.5 | 0.4 | 412 | 74.8 | 40.3 | 41 | 53.2 |
| 2020 | 3040 | 14951 | 14975 | 52.2 | 40 | 5540 | 15152 | 15135 | 51.4 | 50 | 0.8 | 202 | 73.4 | 41.1 | 41 | 51.9 |
| 2021 | 3041 | 2823 | 2844 | 50.4 | 45.5 | 5541 | 3503 | 3484 | 51.5 | 50 | 1 | 681 | 76.4 | 42.6 | 41 | 53.7 |
| 2022 | 3042 | 18075 | 18095 | 50.6 | 47.6 | 5542 | 18231 | 18210 | 52.2 | 45.5 | 1.6 | 157 | 73.6 | 43.9 | 41 | 51.8 |
| 2023 | 3043 | 5 | 23 | 51.3 | 52.6 | 5543 | 269 | 251 | 51.1 | 52.6 | 0.1 | 265 | 76.4 | 46.4 | 41 | 53.9 |
| 2024 | 3044 | 9140 | 9159 | 50.1 | 45 | 5544 | 9249 | 9231 | 50.8 | 47.4 | 0.7 | 110 | 71.3 | 42.7 | 41 | 50 |
| 2025 | 3045 | 24418 | 24439 | 52.9 | 45.5 | 5545 | 24815 | 24791 | 54.5 | 40 | 1.6 | 398 | 75.9 | 43.2 | 41 | 54.1 |
| 2026 | 3046 | 8794 | 8813 | 51.6 | 45 | 5546 | 9358 | 9338 | 51 | 42.9 | 0.6 | 565 | 75.8 | 41.8 | 41 | 53.5 |
| 2027 | 3047 | 24418 | 24439 | 52.9 | 45.5 | 5547 | 24807 | 24786 | 51.7 | 45.5 | 1.2 | 390 | 75.9 | 43.3 | 41 | 53.8 |
| 2028 | 3048 | 2387 | 2405 | 51.6 | 52.6 | 5548 | 3186 | 3165 | 50.4 | 40.9 | 1.2 | 800 | 76.9 | 43.5 | 41 | 54.1 |
| 2029 | 3049 | 24418 | 24439 | 52.9 | 45.5 | 5549 | 24527 | 24506 | 51.7 | 40.9 | 1.2 | 110 | 71.3 | 42.7 | 41 | 50.5 |
| 2030 | 3050 | 24418 | 24439 | 52.9 | 45.5 | 5550 | 24517 | 24494 | 53.2 | 41.7 | 0.3 | 100 | 70.8 | 43 | 41 | 50.5 |
| 2031 | 3051 | 4255 | 4276 | 51.7 | 45.5 | 5551 | 4836 | 4817 | 51.2 | 45 | 0.5 | 582 | 75.9 | 41.9 | 41 | 53.6 |
| 2032 | 3052 | 24420 | 24440 | 50.8 | 42.9 | 5552 | 25082 | 25064 | 51.1 | 52.6 | 0.3 | 663 | 75.7 | 41 | 41 | 53.3 |
| 2033 | 3053 | 8867 | 8887 | 52.3 | 47.6 | 5553 | 9250 | 9232 | 51.6 | 47.4 | 0.8 | 384 | 75.1 | 41.4 | 41 | 53.2 |
| 2034 | 3054 | 14951 | 14975 | 52.2 | 40 | 5554 | 15275 | 15257 | 50.8 | 52.6 | 1.3 | 325 | 74.6 | 40.9 | 41 | 52.6 |
| 2035 | 3055 | 2387 | 2405 | 51.6 | 52.6 | 5555 | 3185 | 3164 | 51 | 45.5 | 0.7 | 799 | 76.9 | 43.6 | 41 | 54.2 |
| 2036 | 3056 | 8865 | 8884 | 50.4 | 45 | 5556 | 9252 | 9235 | 50.1 | 50 | 0.3 | 388 | 75.1 | 41.2 | 41 | 52.7 |
| 2037 | 3057 | 24420 | 24440 | 50.8 | 42.9 | 5557 | 24818 | 24797 | 51.6 | 40.9 | 0.8 | 399 | 75.8 | 42.9 | 41 | 53.4 |
| 2038 | 3058 | 24420 | 24440 | 50.8 | 42.9 | 5558 | 24807 | 24786 | 51.7 | 45.5 | 0.9 | 388 | 75.8 | 43 | 41 | 53.4 |
| 2039 | 3059 | 11541 | 11560 | 50.1 | 45 | 5559 | 12110 | 12090 | 51.1 | 42.9 | 1 | 570 | 75.9 | 41.9 | 41 | 53.3 |
| 2040 | 3060 | 2387 | 2405 | 51.6 | 52.6 | 5560 | 2672 | 2653 | 51.6 | 50 | 0 | 286 | 77 | 47.6 | 41 | 54.5 |
| 2041 | 3061 | 24420 | 24440 | 50.8 | 42.9 | 5561 | 24526 | 24506 | 50.3 | 42.9 | 0.5 | 107 | 70.8 | 42.1 | 41 | 49.8 |
| 2042 | 3062 | 11540 | 11557 | 50.4 | 50 | 5562 | 12110 | 12090 | 51.1 | 42.9 | 0.7 | 571 | 76 | 42 | 41 | 53.4 |
| 2043 | 3063 | 6263 | 6282 | 50.9 | 45 | 5563 | 6483 | 8463 | 50.2 | 42.9 | 0.7 | 221 | 73.7 | 41.2 | 41 | 51.8 |
| 2044 | 3064 | 24418 | 24440 | 55 | 47.8 | 5564 | 24815 | 24791 | 54.5 | 40 | 0.5 | 398 | 75.9 | 43.2 | 41 | 54.6 |
| 2045 | 3065 | 18075 | 118095 | 50.6 | 47.6 | 5565 | 18233 | 18214 | 52 | 50 | 1.4 | 159 | 74 | 44.7 | 41 | 52.1 |
| 2046 | 3066 | 18075 | 18095 | 50.6 | 47.6 | 5566 | 18233 | 18215 | 51.3 | 52.6 | 0.7 | 159 | 74 | 44.7 | 41 | 52.1 |
| 2047 | 3067 | 2429 | 2447 | 50.2 | 47.4 | 5567 | 3055 | 3036 | 50.6 | 50 | 0.4 | 627 | 76.3 | 42.6 | 41 | 53.6 |
| 2048 | 3068 | 19800 | 19818 | 52.1 | 52.6 | 5568 | 19917 | 19896 | 50.9 | 45.5 | 1.2 | 118 | 71.9 | 43.2 | 41 | 50.7 |
| 2049 | 3069 | 24481 | 24500 | 50.1 | 45 | 5569 | 24936 | 24919 | 51.8 | 50 | 1.7 | 456 | 75.7 | 42.1 | 41 | 53.1 |
| 2050 | 3070 | 276 | 294 | 50.5 | 47.4 | 5570 | 713 | 695 | 50.7 | 47.4 | 0.2 | 438 | 79.1 | 50.7 | 41 | 55.7 |
| 2051 | 3071 | 19801 | 19819 | 53.2 | 52.6 | 5571 | 19927 | 19908 | 52.1 | 55 | 1.1 | 127 | 72.7 | 44.1 | 41 | 51.6 |
| 2052 | 3072 | 19801 | 19819 | 53.2 | 52.6 | 5572 | 19925 | 19905 | 51.4 | 52.4 | 1.9 | 125 | 72.5 | 44 | 41 | 51.3 |
| 2053 | 3073 | 3800 | 3824 | 53.6 | 40 | 5573 | 4318 | 4294 | 54.4 | 40 | 0.8 | 519 | 75.3 | 40.8 | 41 | 53.9 |
| 2054 | 3074 | 11540 | 11557 | 50.4 | 50 | 5574 | 12258 | 12238 | 50.3 | 42.9 | 0.2 | 719 | 76.2 | 42 | 41 | 53.5 |
| 2055 | 3075 | 24482 | 24502 | 50.3 | 42.9 | 5575 | 24938 | 24921 | 50.4 | 50 | 0.1 | 457 | 75.6 | 41.8 | 41 | 53.1 |
| 2056 | 3076 | 24482 | 24502 | 50.3 | 42.9 | 5576 | 24807 | 24786 | 51.7 | 45.5 | 1.4 | 326 | 75.4 | 42.9 | 41 | 53 |
| 2057 | 3077 | 8867 | 8888 | 52.7 | 45.5 | 5577 | 9364 | 9346 | 53.9 | 52.6 | 1.2 | 498 | 75.8 | 42.2 | 41 | 54 |
| 2058 | 3078 | 24481 | 24502 | 51.5 | 45.5 | 5578 | 25080 | 25062 | 53.5 | 52.6 | 2 | 600 | 75.5 | 40.8 | 41 | 53.4 |
| 2059 | 3079 | 8865 | 8884 | 50.4 | 45 | 5579 | 9107 | 9086 | 51.6 | 45.5 | 1.2 | 243 | 74 | 41.2 | 41 | 52 |
| 2060 | 3080 | 8867 | 8888 | 52.7 | 45.5 | 5580 | 9313 | 9293 | 52.1 | 47.6 | 0.6 | 447 | 75.5 | 41.6 | 41 | 53.5 |
| 2061 | 3081 | 2427 | 2445 | 52.1 | 52.6 | 5581 | 3055 | 3036 | 50.6 | 50 | 1.5 | 629 | 76.4 | 42.8 | 41 | 53.7 |
| 2062 | 3082 | 2823 | 2844 | 50.4 | 45.5 | 5582 | 3504 | 3485 | 50.4 | 45 | 0.1 | 682 | 76.3 | 42.5 | 41 | 53.7 |
| 2063 | 3083 | 24483 | 24503 | 51 | 42.9 | 5583 | 25085 | 25068 | 50.3 | 50 | 0.6 | 603 | 75.4 | 40.6 | 41 | 53 |
| 2064 | 3084 | 15255 | 15273 | 50.3 | 52.6 | 5584 | 15649 | 15632 | 50.1 | 50 | 0.2 | 395 | 75.1 | 41.3 | 41 | 52.7 |
| 2065 | 3085 | 24483 | 24503 | 51 | 42.9 | 5585 | 25082 | 25064 | 51.1 | 52.6 | 0.1 | 600 | 75.5 | 40.8 | 41 | 53.3 |
| 2066 | 3086 | 8867 | 8886 | 50.7 | 50 | 5586 | 9375 | 9354 | 50.4 | 40.9 | 0.3 | 509 | 75.7 | 41.8 | 41 | 53.2 |
| 2067 | 3087 | 12976 | 12994 | 50.3 | 47.4 | 5587 | 13329 | 13308 | 50.5 | 40.9 | 0.2 | 354 | 76.1 | 44.1 | 41 | 53.4 |
| 2068 | 3088 | 24483 | 24503 | 51 | 42.9 | 5588 | 25081 | 25063 | 52.4 | 52.6 | 1.4 | 599 | 75.5 | 40.7 | 41 | 53.2 |
| 2069 | 3089 | 379 | 398 | 50.1 | 45 | 5589 | 941 | 922 | 50.5 | 50 | 0.4 | 563 | 78.7 | 48.8 | 41 | 55.2 |
| 2070 | 3090 | 24483 | 24503 | 51 | 42.9 | 5590 | 24936 | 24919 | 51.8 | 50 | 0.8 | 454 | 75.7 | 42.1 | 41 | 53.4 |
| 2071 | 3091 | 19802 | 19820 | 53 | 52.6 | 5591 | 19927 | 19908 | 52.1 | 55 | 0.8 | 126 | 72.8 | 44.4 | 41 | 51.7 |
| 2072 | 3092 | 9934 | 9953 | 50.7 | 50 | 5592 | 10670 | 10649 | 51.3 | 40.9 | 0.6 | 737 | 75.7 | 40.8 | 41 | 53.3 |
| 2073 | 3093 | 8866 | 8885 | 51.1 | 45 | 5593 | 9312 | 9293 | 50.6 | 45 | 0.5 | 447 | 75.4 | 41.4 | 41 | 53 |
| 2074 | 3094 | 19846 | 19866 | 51.2 | 42.9 | 5594 | 20033 | 20016 | 50.4 | 50 | 0.8 | 188 | 74.2 | 43.6 | 41 | 52.2 |
| 2075 | 3095 | 19848 | 19867 | 50.7 | 45 | 5595 | 20033 | 20016 | 50.4 | 50 | 0.3 | 186 | 74.1 | 43.5 | 41 | 52.1 |
| 2076 | 3096 | 9538 | 9558 | 50.9 | 42.9 | 5596 | 10017 | 9999 | 52.8 | 52.6 | 1.9 | 480 | 75.5 | 41.5 | 41 | 53.2 |
| 2077 | 3097 | 8220 | 8238 | 51.5 | 47.4 | 5597 | 8933 | 8916 | 52.2 | 50 | 0.7 | 714 | 75.4 | 40.1 | 41 | 53.3 |
| 2078 | 3098 | 9140 | 9159 | 50.1 | 45 | 5598 | 9249 | 9232 | 50 | 50 | 0.1 | 110 | 71.3 | 42.7 | 41 | 50 |
| 2079 | 3099 | 12976 | 12994 | 50.3 | 47.4 | 5599 | 13332 | 13312 | 50.9 | 47.6 | 0.6 | 357 | 76.2 | 44.3 | 41 | 53.5 |
| 2080 | 3100 | 15752 | 15772 | 50.8 | 47.6 | 5600 | 16174 | 16154 | 50.4 | 42.9 | 0.4 | 423 | 75.1 | 40.9 | 41 | 52.8 |
| 2081 | 3101 | 18074 | 18094 | 51.1 | 42.9 | 5601 | 18232 | 18212 | 50.6 | 47.6 | 0.5 | 159 | 73.7 | 44 | 41 | 51.9 |
| 2082 | 3102 | 24559 | 24579 | 52 | 52.4 | 5602 | 25081 | 25063 | 52.4 | 52.6 | 0.4 | 523 | 75.5 | 41.1 | 41 | 53.5 |
| 2083 | 3103 | 24559 | 24579 | 52 | 52.4 | 5603 | 25079 | 25061 | 52.7 | 52.6 | 0.7 | 521 | 75.5 | 41.3 | 41 | 53.6 |
| 2084 | 3104 | 3169 | 3191 | 52.1 | 47.8 | 5604 | 3650 | 3631 | 53.1 | 50 | 1 | 482 | 75.9 | 42.3 | 41 | 53.8 |
| 2085 | 3105 | 28117 | 28135 | 50.6 | 52.6 | 5605 | 28672 | 28654 | 50.6 | 52.6 | 0.1 | 556 | 80 | 52 | 41 | 56.3 |
| 2086 | 3106 | 1809 | 1829 | 50.6 | 42.9 | 5606 | 2103 | 2082 | 52 | 45.5 | 1.5 | 295 | 75.4 | 43.4 | 41 | 53 |
| 2087 | 3107 | 24559 | 24579 | 52 | 52.4 | 5607 | 24933 | 24913 | 51.1 | 42.9 | 0.8 | 375 | 75.6 | 42.7 | 41 | 53.4 |
| 2088 | 3108 | 1809 | 1829 | 50.6 | 42.9 | 5608 | 2113 | 2094 | 50.1 | 45 | 0.4 | 305 | 75.4 | 43.3 | 41 | 52.9 |
| 2089 | 3109 | 28116 | 28134 | 50.8 | 47.4 | 5609 | 28505 | 28487 | 50.2 | 47.4 | 0.6 | 390 | 79.4 | 51.8 | 41 | 55.8 |
| 2090 | 3110 | 1808 | 1828 | 50.6 | 42.9 | 5610 | 2103 | 2082 | 52 | 45.5 | 1.5 | 296 | 75.5 | 43.6 | 41 | 53.1 |
| 2091 | 3111 | 15951 | 15975 | 53.1 | 40 | 5611 | 16210 | 16192 | 54.3 | 52.6 | 1.2 | 260 | 74.3 | 41.5 | 41 | 53.1 |
| 2092 | 3112 | 8865 | 8884 | 50.4 | 45 | 5612 | 9341 | 9322 | 51.1 | 50 | 0.7 | 477 | 75.6 | 41.7 | 41 | 53.1 |
| 2093 | 3113 | 15 | 33 | 50.7 | 52.6 | 5613 | 642 | 622 | 51.6 | 47.6 | 0.9 | 628 | 79 | 49.2 | 41 | 55.6 |
| 2094 | 3114 | 8861 | 8880 | 50.2 | 45 | 5614 | 9107 | 9086 | 51.6 | 45.5 | 1.4 | 247 | 73.9 | 40.9 | 41 | 51.9 |
| 2095 | 3115 | 1808 | 1828 | 50.6 | 42.9 | 5615 | 2113 | 2094 | 50.1 | 45 | 0.4 | 306 | 75.5 | 43.5 | 41 | 53 |
| 2096 | 3116 | 24562 | 24580 | 50.1 | 52.6 | 5616 | 24933 | 24913 | 51.1 | 42.9 | 1.1 | 372 | 75.5 | 42.5 | 41 | 53 |
| 2097 | 3117 | 28116 | 28134 | 50.8 | 47.4 | 5617 | 28672 | 28654 | 50.6 | 52.6 | 0.2 | 557 | 80 | 51.9 | 41 | 56.2 |
| 2098 | 3118 | 24560 | 24580 | 51.3 | 52.4 | 5618 | 25081 | 25063 | 52.4 | 52.6 | 1.1 | 522 | 75.4 | 41 | 41 | 53.3 |
| 2099 | 3119 | 24560 | 24580 | 51.3 | 52.4 | 5619 | 25079 | 25061 | 52.7 | 52.6 | 1.4 | 520 | 75.5 | 41.2 | 41 | 53.3 |
| 2100 | 3120 | 16366 | 16384 | 50.3 | 52.6 | 5620 | 16775 | 16755 | 51.1 | 42.9 | 0.7 | 410 | 75.1 | 41 | 41 | 52.7 |
| 2101 | 3121 | 16366 | 16384 | 50.3 | 52.6 | 5621 | 16774 | 16754 | 50.4 | 42.9 | 0.1 | 409 | 75.1 | 41.1 | 41 | 52.8 |
| 2102 | 3122 | 24569 | 24590 | 56.6 | 54.5 | 5622 | 25089 | 25070 | 55.8 | 55 | 0.8 | 521 | 75.4 | 40.9 | 41 | 54.6 |
| 2103 | 3123 | 24569 | 24590 | 56.6 | 54.5 | 5623 | 25088 | 25069 | 55 | 50 | 1.6 | 520 | 75.3 | 40.8 | 41 | 54.3 |
| 2104 | 3124 | 24567 | 24590 | 57.8 | 54.2 | 5624 | 25095 | 25072 | 59.3 | 54.2 | 1.5 | 529 | 75.4 | 40.8 | 41 | 55.2 |
| 2105 | 3125 | 24568 | 24591 | 58.9 | 54.2 | 5625 | 25095 | 25072 | 59.3 | 54.2 | 0.4 | 528 | 75.3 | 40.7 | 41 | 55.5 |
| 2106 | 3126 | 24568 | 24591 | 58.9 | 54.2 | 5626 | 25091 | 25070 | 59.1 | 54.5 | 0.2 | 524 | 75.4 | 40.8 | 41 | 55.5 |
| 2107 | 3127 | 24568 | 24591 | 58.9 | 54.2 | 5627 | 25090 | 25069 | 58.3 | 54.5 | 0.6 | 523 | 75.4 | 40.9 | 41 | 55.4 |
| 2108 | 3128 | 16366 | 16384 | 50.3 | 52.6 | 5628 | 16774 | 16753 | 51.1 | 40.9 | 0.8 | 409 | 75.1 | 41.1 | 41 | 52.8 |
| 2109 | 3129 | 1806 | 1825 | 51.1 | 45 | 5629 | 2103 | 2082 | 52 | 45.5 | 1 | 298 | 75.5 | 43.6 | 41 | 53.3 |
| 2110 | 3130 | 8374 | 8395 | 52.4 | 45.5 | 5630 | 9107 | 9086 | 51.6 | 45.5 | 0.8 | 734 | 75.5 | 40.2 | 41 | 53.4 |
| 2111 | 3131 | 24622 | 24643 | 57.1 | 54.5 | 5631 | 24935 | 24913 | 56.1 | 47.8 | 1 | 314 | 74.5 | 40.8 | 41 | 54.1 |
| 2112 | 3132 | 9130 | 9150 | 51.3 | 42.9 | 5632 | 9358 | 9338 | 51 | 42.9 | 0.3 | 229 | 74.5 | 42.8 | 41 | 52.5 |
| 2113 | 3133 | 12936 | 12957 | 53.7 | 45.5 | 5633 | 13530 | 13511 | 55.6 | 55 | 1.9 | 595 | 77.4 | 45.4 | 41 | 55.4 |
| 2114 | 3134 | 8373 | 8391 | 50.7 | 47.4 | 5634 | 9107 | 9086 | 51.6 | 45.5 | 0.9 | 735 | 75.4 | 40.1 | 41 | 53.1 |
| 2115 | 3135 | 1352 | 1371 | 56.1 | 55 | 5635 | 1701 | 1678 | 54.3 | 41.7 | 1.8 | 350 | 76.7 | 45.7 | 41 | 55.1 |
| 2116 | 3136 | 8867 | 8886 | 50.7 | 50 | 5636 | 9342 | 9323 | 52.1 | 50 | 1.4 | 476 | 75.7 | 42 | 41 | 53.3 |
| 2117 | 3137 | 1352 | 1371 | 56.1 | 55 | 5637 | 1701 | 1677 | 54.7 | 40 | 1.4 | 350 | 76.7 | 45.7 | 41 | 55.2 |
| 2118 | 3138 | 9130 | 9150 | 51.3 | 42.9 | 5638 | 9249 | 9232 | 50 | 50 | 1.3 | 120 | 71.7 | 42.5 | 41 | 50.3 |
| 2119 | 3139 | 16861 | 16880 | 50.8 | 50 | 5639 | 17062 | 17045 | 50.2 | 50 | 0.6 | 202 | 74.8 | 44.6 | 41 | 52.5 |
| 2120 | 3140 | 9130 | 9150 | 51.3 | 42.9 | 5640 | 9249 | 9231 | 50.8 | 47.4 | 0.5 | 120 | 71.7 | 42.5 | 41 | 50.5 |
| 2121 | 3141 | 9130 | 9150 | 51.3 | 42.9 | 5641 | 9249 | 9230 | 51.5 | 45 | 0.2 | 120 | 71.7 | 42.5 | 41 | 50.7 |
| 2122 | 3142 | 8372 | 8390 | 50.7 | 47.4 | 5642 | 9060 | 9039 | 50.3 | 40.9 | 0.4 | 689 | 75.4 | 40.2 | 41 | 53 |
| 2123 | 3143 | 18074 | 18093 | 50.3 | 45 | 5643 | 18232 | 18212 | 50.6 | 47.6 | 0.3 | 159 | 73.7 | 44 | 41 | 51.8 |
| 2124 | 3144 | 2671 | 2692 | 52.1 | 40.9 | 5644 | 3193 | 3172 | 52.6 | 50 | 0.5 | 523 | 75.8 | 41.9 | 41 | 53.8 |
| 2125 | 3145 | 16562 | 16581 | 52.6 | 50 | 5645 | 17064 | 17045 | 51.4 | 50 | 1.2 | 503 | 75.8 | 42.1 | 41 | 53.6 |
| 2126 | 3146 | 2671 | 2692 | 52.1 | 40.9 | 5646 | 3193 | 3173 | 51.4 | 47.6 | 0.7 | 523 | 75.8 | 41.9 | 41 | 53.6 |
| 2127 | 3147 | 8372 | 8390 | 50.7 | 47.4 | 5647 | 9107 | 9086 | 51.6 | 45.5 | 0.9 | 736 | 75.5 | 40.2 | 41 | 53.1 |
| 2128 | 3148 | 12726 | 12746 | 51.3 | 47.6 | 5648 | 13321 | 13301 | 50.3 | 42.9 | 0.9 | 596 | 76.7 | 43.6 | 41 | 53.9 |
| 2129 | 3149 | 8867 | 8886 | 50.7 | 50 | 5649 | 9312 | 9293 | 50.6 | 45 | 0.1 | 446 | 75.4 | 41.5 | 41 | 53 |
| 2130 | 3150 | 16562 | 16580 | 51.9 | 52.6 | 5650 | 17062 | 17045 | 50.2 | 50 | 1.7 | 501 | 75.8 | 42.1 | 41 | 53.2 |
| 2131 | 3151 | 27377 | 27397 | 53.4 | 47.6 | 5651 | 27674 | 27653 | 52.5 | 40.9 | 0.9 | 298 | 74.3 | 40.6 | 41 | 52.8 |
| 2132 | 3152 | 16556 | 16573 | 50.3 | 50 | 5652 | 17111 | 17090 | 51.1 | 40.9 | 0.8 | 556 | 76.1 | 42.4 | 41 | 53.5 |
| 2133 | 3153 | 7815 | 7833 | 51.5 | 52.6 | 5653 | 8531 | 8512 | 52 | 45 | 0.5 | 717 | 75.7 | 40.7 | 41 | 53.5 |
| 2134 | 3154 | 3223 | 3241 | 50.2 | 52.6 | 5654 | 3494 | 3473 | 50.4 | 40.9 | 0.2 | 272 | 74.6 | 41.9 | 41 | 52.4 |
| 2135 | 3155 | 8372 | 8390 | 50.7 | 47.4 | 5655 | 9109 | 9087 | 50.5 | 43.5 | 0.1 | 738 | 75.4 | 40.1 | 41 | 53.1 |
| 2136 | 3156 | 3041 | 3065 | 57.7 | 48 | 5656 | 3650 | 3628 | 56.3 | 47.8 | 1.4 | 610 | 76.3 | 42.8 | 41 | 55.4 |
| 2137 | 3157 | 9569 | 9591 | 53 | 43.5 | 5657 | 10017 | 9999 | 52.8 | 52.6 | 0.3 | 449 | 75.4 | 41.4 | 41 | 53.7 |
| 2138 | 3158 | 3041 | 3065 | 57.7 | 48 | 5658 | 3649 | 3625 | 56.6 | 44 | 1.2 | 609 | 76.3 | 42.7 | 41 | 55.5 |
| 2139 | 3159 | 13176 | 13196 | 51.4 | 47.6 | 5659 | 13321 | 13301 | 50.3 | 42.9 | 1 | 146 | 73.3 | 43.8 | 41 | 51.5 |
| 2140 | 3160 | 16366 | 16385 | 52.9 | 55 | 5660 | 16775 | 16755 | 51.1 | 42.9 | 1.8 | 410 | 75.1 | 41 | 41 | 53 |
| 2141 | 3161 | 16366 | 16385 | 52.9 | 55 | 5661 | 16775 | 16754 | 51.7 | 40.9 | 1.1 | 410 | 75.1 | 41 | 41 | 53.2 |
| 2142 | 3162 | 16366 | 16385 | 52.9 | 55 | 5662 | 16774 | 16753 | 51.1 | 40.9 | 1.8 | 409 | 75.1 | 41.1 | 41 | 53 |
| 2143 | 3163 | 1402 | 1422 | 50.2 | 42.9 | 5663 | 2104 | 2084 | 50.6 | 42.9 | 0.4 | 703 | 76.7 | 43.2 | 41 | 53.8 |
| 2144 | 3164 | 1402 | 1422 | 50.2 | 42.9 | 5664 | 1697 | 1678 | 50.3 | 45 | 0.1 | 296 | 76 | 44.9 | 41 | 53.4 |
| 2145 | 3165 | 3055 | 3076 | 52.4 | 45.5 | 5665 | 3503 | 3484 | 51.5 | 50 | 1 | 449 | 76.1 | 43.2 | 41 | 53.8 |
| 2146 | 3166 | 15211 | 15230 | 50.2 | 45 | 5666 | 16001 | 15980 | 51.1 | 45.5 | 0.9 | 791 | 75.6 | 40.3 | 41 | 53.1 |
| 2147 | 3167 | 12267 | 12290 | 54.5 | 41.7 | 5667 | 12414 | 12392 | 53.9 | 43.5 | 0.6 | 148 | 72.2 | 41.2 | 41 | 51.8 |
| 2148 | 3168 | 8861 | 8880 | 50.2 | 45 | 5668 | 9256 | 9237 | 50.8 | 45 | 0.6 | 396 | 75 | 40.9 | 41 | 52.6 |
| 2149 | 3169 | 3049 | 3071 | 56.3 | 52.2 | 5669 | 3650 | 3628 | 56.3 | 47.8 | 0 | 602 | 76.4 | 42.9 | 41 | 55.4 |
| 2150 | 3170 | 3049 | 3071 | 56.3 | 52.2 | 5670 | 3648 | 3625 | 55.5 | 41.7 | 0.8 | 600 | 76.3 | 42.7 | 41 | 55.2 |
| 2151 | 3171 | 8861 | 8880 | 50.2 | 45 | 5671 | 9313 | 9294 | 50.4 | 50 | 0.3 | 453 | 75.3 | 41.3 | 41 | 52.9 |
| 2152 | 3172 | 12352 | 12375 | 52.9 | 41.7 | 5672 | 12911 | 12891 | 51.2 | 47.6 | 1.7 | 560 | 76.1 | 42.5 | 41 | 53.7 |
| 2153 | 3173 | 7965 | 7985 | 51.9 | 42.9 | 5673 | 8531 | 8512 | 52 | 45 | 0.2 | 567 | 75.1 | 40 | 41 | 53.2 |
| 2154 | 3174 | 18017 | 18036 | 54.8 | 55 | 5674 | 18233 | 18212 | 53.5 | 50 | 1.3 | 217 | 74.7 | 43.8 | 41 | 53.5 |
| 2155 | 3175 | 8867 | 8886 | 50.7 | 50 | 5675 | 9257 | 9238 | 50.5 | 45 | 0.2 | 391 | 75.1 | 41.2 | 41 | 52.8 |
| 2156 | 3176 | 3221 | 3239 | 51.5 | 52.6 | 5676 | 3494 | 3473 | 50.4 | 40.9 | 1.1 | 274 | 74.5 | 41.6 | 41 | 52.4 |
| 2157 | 3177 | 1402 | 1422 | 50.2 | 42.9 | 5677 | 1697 | 1677 | 51 | 42.9 | 0.8 | 296 | 76 | 44.9 | 41 | 53.4 |
| 2158 | 3178 | 1402 | 1422 | 50.2 | 42.9 | 5678 | 1697 | 1676 | 51.7 | 40.9 | 1.5 | 296 | 76 | 44.9 | 41 | 53.4 |
| 2159 | 3179 | 18011 | 18032 | 55.7 | 54.5 | 5679 | 18220 | 18201 | 56.1 | 55 | 0.4 | 210 | 74.5 | 43.3 | 41 | 53.9 |
| 2160 | 3180 | 12726 | 12746 | 51.3 | 47.6 | 5680 | 13329 | 13308 | 50.5 | 40.9 | 0.8 | 604 | 76.6 | 43.5 | 41 | 53.9 |
| 2161 | 3181 | 1402 | 1425 | 52.8 | 41.7 | 5681 | 1698 | 1678 | 51.7 | 42.9 | 1.1 | 297 | 76 | 44.8 | 41 | 53.8 |
| 2162 | 3182 | 18013 | 18032 | 52.2 | 55 | 5682 | 18223 | 18205 | 53.3 | 52.6 | 1.1 | 211 | 74.4 | 43.1 | 41 | 52.8 |
| 2163 | 3183 | 3777 | 3797 | 51.7 | 47.6 | 5683 | 4444 | 4424 | 50.6 | 42.9 | 1.1 | 668 | 75.6 | 40.7 | 41 | 53.2 |
| 2164 | 3184 | 3777 | 3797 | 51.7 | 47.6 | 5684 | 4445 | 4425 | 50.6 | 42.9 | 1.1 | 669 | 75.6 | 40.7 | 41 | 53.2 |
| 2165 | 3185 | 7876 | 7895 | 51.5 | 45 | 5685 | 8189 | 8170 | 50.6 | 50 | 0.9 | 314 | 75.1 | 42.4 | 41 | 52.9 |
| 2166 | 3186 | 18014 | 18032 | 51 | 52.6 | 5686 | 18229 | 18209 | 50.1 | 42.9 | 0.8 | 216 | 74.2 | 42.6 | 41 | 52.1 |
| 2167 | 3187 | 1402 | 1425 | 52.8 | 41.7 | 5687 | 1697 | 1677 | 51 | 42.9 | 1.8 | 296 | 76 | 44.9 | 41 | 53.6 |
| 2168 | 3188 | 1402 | 1425 | 52.8 | 41.7 | 5688 | 1697 | 1676 | 51.7 | 40.9 | 1.1 | 296 | 76 | 44.9 | 41 | 53.8 |
| 2169 | 3189 | 1402 | 1425 | 52.8 | 41.7 | 5689 | 1501 | 1481 | 51.2 | 42.9 | 1.6 | 100 | 72 | 46 | 41 | 50.9 |
| 2170 | 3190 | 12366 | 12384 | 51.7 | 52.6 | 5690 | 13155 | 13138 | 50.4 | 50 | 1.3 | 790 | 76.8 | 43.4 | 41 | 54 |
| 2171 | 3191 | 27361 | 27380 | 52.4 | 55 | 5691 | 27573 | 27552 | 52.3 | 40.9 | 0.1 | 213 | 75 | 44.6 | 41 | 53.3 |
| 2172 | 3192 | 18006 | 18028 | 54.5 | 52.2 | 5692 | 18220 | 18201 | 56.1 | 55 | 1.6 | 215 | 74.5 | 43.3 | 41 | 53.6 |
| 2173 | 3193 | 1442 | 1461 | 51.6 | 55 | 5693 | 1872 | 1854 | 53.2 | 52.6 | 1.6 | 431 | 76.2 | 43.6 | 41 | 53.9 |
| 2174 | 3194 | 27361 | 27380 | 52.4 | 55 | 5694 | 27567 | 27547 | 51.1 | 42.9 | 1.3 | 207 | 75.1 | 44.9 | 41 | 53 |
| 2175 | 3195 | 9131 | 9151 | 50.4 | 42.9 | 5695 | 9249 | 9230 | 51.5 | 45 | 1.2 | 119 | 71.8 | 42.9 | 41 | 50.5 |
| 2176 | 3196 | 3217 | 3236 | 51.1 | 50 | 5696 | 3504 | 3485 | 50.4 | 45 | 0.7 | 288 | 74.8 | 42 | 41 | 52.6 |
| 2177 | 3197 | 18011 | 18029 | 51.3 | 52.6 | 5697 | 18232 | 18212 | 50.6 | 47.6 | 0.7 | 222 | 74.8 | 43.7 | 41 | 52.6 |
| 2178 | 3198 | 3055 | 3074 | 51.1 | 50 | 5698 | 3503 | 3484 | 51.5 | 50 | 0.4 | 449 | 76.1 | 43.2 | 41 | 53.7 |
| 2179 | 3199 | 8866 | 8886 | 52.3 | 47.6 | 5699 | 9364 | 9346 | 53.9 | 52.6 | 1.6 | 499 | 75.8 | 42.1 | 41 | 53.9 |
| 2180 | 3200 | 16368 | 16387 | 50.2 | 45 | 5700 | 16774 | 16752 | 52.2 | 43.5 | 2 | 407 | 75 | 40.8 | 41 | 52.7 |
| 2181 | 3201 | 8859 | 8879 | 50 | 42.9 | 5701 | 9252 | 9235 | 50.1 | 50 | 0.1 | 394 | 75 | 41.1 | 41 | 52.6 |
| 2182 | 3202 | 9131 | 9151 | 50.4 | 42.9 | 5702 | 9249 | 9231 | 50.8 | 47.4 | 0.5 | 119 | 71.8 | 42.9 | 41 | 50.5 |
| 2183 | 3203 | 3217 | 3236 | 51.1 | 50 | 5703 | 3494 | 3473 | 50.4 | 40.9 | 0.7 | 278 | 74.6 | 41.7 | 41 | 52.4 |
| 2184 | 3204 | 8859 | 8879 | 50 | 42.9 | 5704 | 9341 | 9322 | 51.1 | 50 | 1.1 | 483 | 75.6 | 41.6 | 41 | 53 |
| 2185 | 3205 | 9131 | 9151 | 50.4 | 42.9 | 5705 | 9249 | 9232 | 50 | 50 | 0.3 | 119 | 71.8 | 42.9 | 41 | 50.4 |
| 2186 | 3206 | 8867 | 8886 | 50.7 | 50 | 5706 | 9248 | 9229 | 50.1 | 45 | 0.5 | 382 | 75.1 | 41.4 | 41 | 52.7 |
| 2187 | 3207 | 27366 | 27384 | 52.2 | 52.6 | 5707 | 27576 | 27555 | 51 | 40.9 | 1.2 | 211 | 74.8 | 44.1 | 41 | 52.7 |
| 2188 | 3208 | 1442 | 1461 | 51.6 | 55 | 5708 | 1879 | 1861 | 53 | 52.6 | 1.4 | 438 | 76.2 | 43.6 | 41 | 54 |
| 2189 | 3209 | 12366 | 12384 | 51.7 | 52.6 | 5709 | 12724 | 12705 | 52.4 | 55 | 0.7 | 359 | 75.6 | 42.9 | 41 | 53.5 |
| 2190 | 3210 | 12366 | 12384 | 51.7 | 52.6 | 5710 | 12498 | 12480 | 50 | 47.4 | 1.6 | 133 | 73 | 44.4 | 41 | 51.2 |
| 2191 | 3211 | 98 | 118 | 50.6 | 42.9 | 5711 | 713 | 695 | 50.7 | 47.4 | 0.1 | 616 | 79 | 49.4 | 41 | 55.6 |
| 2192 | 3212 | 12373 | 12391 | 50.8 | 47.4 | 5712 | 13155 | 13138 | 50.4 | 50 | 0.4 | 783 | 76.8 | 43.4 | 41 | 54 |
| 2193 | 3213 | 18011 | 18030 | 52.9 | 55 | 5713 | 18230 | 18209 | 51.3 | 45.5 | 1.6 | 220 | 74.5 | 43.2 | 41 | 52.7 |
| 2194 | 3214 | 1402 | 1426 | 54.1 | 40 | 5714 | 1700 | 1676 | 53.9 | 40 | 0.2 | 299 | 76 | 44.8 | 41 | 54.5 |
| 2195 | 3215 | 18011 | 18030 | 52.9 | 55 | 5715 | 18223 | 18205 | 53.3 | 52.6 | 0.5 | 213 | 74.4 | 43.2 | 41 | 53.1 |
| 2196 | 3216 | 1402 | 1426 | 54.1 | 40 | 5716 | 1698 | 1677 | 52.3 | 40.9 | 1.8 | 297 | 76 | 44.8 | 41 | 54 |
| 2197 | 3217 | 16463 | 16483 | 51.3 | 42.9 | 5717 | 17032 | 17011 | 52 | 45.5 | 0.7 | 570 | 76 | 42.1 | 41 | 53.7 |
| 2198 | 3218 | 18009 | 18030 | 54.6 | 54.5 | 5718 | 18220 | 18201 | 56.1 | 55 | 1.6 | 212 | 74.5 | 43.4 | 41 | 53.6 |
| 2199 | 3219 | 1402 | 1426 | 54.1 | 40 | 5719 | 1700 | 1678 | 52.9 | 43.5 | 1.3 | 299 | 76 | 44.8 | 41 | 54.2 |
| 2200 | 3220 | 9131 | 9151 | 50.4 | 42.9 | 5720 | 9358 | 9338 | 51 | 42.9 | 0.6 | 228 | 74.6 | 43 | 41 | 52.4 |
| 2201 | 3221 | 3055 | 3075 | 51.8 | 47.6 | 5721 | 3503 | 3484 | 51.5 | 50 | 0.3 | 449 | 76.1 | 43.2 | 41 | 53.8 |
| 2202 | 3222 | 18013 | 18031 | 50.6 | 52.6 | 5722 | 18232 | 18212 | 50.6 | 47.6 | 0 | 220 | 74.7 | 43.6 | 41 | 52.6 |
| 2203 | 3223 | 8794 | 8813 | 51.6 | 45 | 5723 | 9249 | 9230 | 51.5 | 45 | 0.1 | 456 | 75.4 | 41.4 | 41 | 53.4 |
| 2204 | 3224 | 8794 | 8813 | 51.6 | 45 | 5724 | 9249 | 9231 | 50.8 | 47.4 | 0.8 | 456 | 75.4 | 41.4 | 41 | 53.1 |
| 2205 | 3225 | 16549 | 16567 | 54.9 | 52.6 | 5725 | 17065 | 17045 | 53.1 | 47.6 | 1.9 | 517 | 76 | 42.4 | 41 | 54.2 |
| 2206 | 3226 | 8794 | 8813 | 51.6 | 45 | 5726 | 9249 | 9232 | 50 | 50 | 1.6 | 456 | 75.4 | 41.4 | 41 | 52.9 |
| 2207 | 3227 | 1402 | 1426 | 54.1 | 40 | 5727 | 2104 | 2082 | 53.5 | 43.5 | 0.6 | 703 | 76.7 | 43.2 | 41 | 54.8 |
| 2208 | 3228 | 9927 | 9946 | 51.3 | 50 | 5728 | 10356 | 10336 | 52.4 | 47.6 | 1.1 | 430 | 75.6 | 42.1 | 41 | 53.4 |
| 2209 | 3229 | 3219 | 3238 | 50.7 | 50 | 5729 | 3494 | 3473 | 50.4 | 40.9 | 0.3 | 276 | 74.5 | 41.7 | 41 | 52.4 |
| 2210 | 3230 | 16549 | 16567 | 54.9 | 52.6 | 5730 | 17033 | 17011 | 53.2 | 43.5 | 1.7 | 485 | 75.8 | 42.1 | 41 | 54.1 |
| 2211 | 3231 | 18014 | 18032 | 51 | 52.6 | 5731 | 18702 | 18685 | 50.2 | 50 | 0.8 | 689 | 76.2 | 42.2 | 41 | 53.5 |
| 2212 | 3232 | 8794 | 8813 | 51.6 | 45 | 5732 | 9333 | 9315 | 52.2 | 52.6 | 0.6 | 540 | 75.9 | 42 | 41 | 53.7 |
| 2213 | 3233 | 8867 | 8888 | 52.7 | 45.5 | 5733 | 9249 | 9229 | 53 | 47.6 | 0.2 | 383 | 75.2 | 41.5 | 41 | 53.5 |
| 2214 | 3234 | 18009 | 18028 | 51.6 | 55 | 5734 | 18229 | 18209 | 50.1 | 42.9 | 1.5 | 221 | 74.5 | 43 | 41 | 52.3 |
| 2215 | 3235 | 9633 | 9651 | 51 | 47.4 | 5735 | 10017 | 9999 | 52.8 | 52.6 | 1.8 | 385 | 75.6 | 42.6 | 41 | 53.3 |
| 2216 | 3236 | 9915 | 9935 | 51.8 | 47.6 | 5736 | 10449 | 10428 | 51.9 | 40.9 | 0.1 | 535 | 75.4 | 40.9 | 41 | 53.4 |
| 2217 | 3237 | 29259 | 29277 | 50.9 | 52.6 | 5737 | 29414 | 29395 | 50.5 | 50 | 0.3 | 156 | 74.5 | 46.2 | 41 | 52.4 |
| 2218 | 3238 | 8868 | 8889 | 50.4 | 40.9 | 5738 | 9317 | 9297 | 50.5 | 42.9 | 0.1 | 450 | 75.4 | 41.6 | 41 | 53 |
| 2219 | 3239 | 29257 | 29276 | 51.3 | 50 | 5739 | 29414 | 29395 | 50.5 | 50 | 0.8 | 158 | 74.6 | 46.2 | 41 | 52.5 |
| 2220 | 3240 | 13176 | 13196 | 51.4 | 47.6 | 5740 | 13332 | 13312 | 50.9 | 47.6 | 0.5 | 157 | 73.6 | 43.9 | 41 | 51.9 |
| 2221 | 3241 | 9918 | 9938 | 51.4 | 47.6 | 5741 | 10449 | 10428 | 51.9 | 40.9 | 0.5 | 53.2 | 75.4 | 40.8 | 41 | 53.3 |
| 2222 | 3242 | 13176 | 13196 | 51.4 | 47.6 | 5742 | 13856 | 13835 | 50.1 | 45.5 | 1.3 | 681 | 75.8 | 41.1 | 41 | 53.2 |
| 2223 | 3243 | 29253 | 29270 | 50 | 50 | 5743 | 29414 | 29395 | 50.5 | 50 | 0.5 | 162 | 75.2 | 47.5 | 41 | 52.8 |
| 2224 | 3244 | 13037 | 13058 | 54.8 | 50 | 5744 | 13530 | 13511 | 55.6 | 55 | 0.8 | 494 | 77.3 | 45.7 | 41 | 55.6 |
| 2225 | 3245 | 18009 | 18028 | 51.6 | 55 | 5745 | 18702 | 18685 | 50.2 | 50 | 1.5 | 694 | 76.3 | 42.4 | 41 | 53.6 |
| 2226 | 3246 | 24178 | 24197 | 50.3 | 40 | 5746 | 24938 | 24921 | 50.4 | 50 | 0.1 | 761 | 75.8 | 40.9 | 41 | 53.2 |
| 2227 | 3247 | 24174 | 24195 | 52.5 | 40.9 | 5747 | 24740 | 24717 | 52.5 | 41.7 | 0 | 567 | 76 | 42.2 | 41 | 54 |
| 2228 | 3248 | 7679 | 7698 | 50.6 | 50 | 5748 | 8054 | 8035 | 50.4 | 50 | 0.1 | 376 | 75.6 | 42.6 | 41 | 53.1 |
| 2229 | 3249 | 18005 | 18024 | 51.1 | 50 | 5749 | 18229 | 18209 | 50.1 | 42.9 | 1 | 225 | 74.4 | 42.7 | 41 | 52.2 |
| 2230 | 3250 | 24174 | 24195 | 52.5 | 40.9 | 5750 | 24933 | 24913 | 51.1 | 42.9 | 1.4 | 760 | 75.8 | 40.9 | 41 | 53.5 |
| 2231 | 3251 | 3016 | 3036 | 50.2 | 42.9 | 5751 | 3500 | 3481 | 51.2 | 50 | 0.9 | 485 | 76.3 | 43.3 | 41 | 53.6 |
| 2232 | 3252 | 28820 | 28838 | 53.7 | 52.6 | 5752 | 29306 | 29288 | 53.5 | 52.6 | 0.2 | 487 | 77.1 | 45.4 | 41 | 55.1 |
| 2233 | 3253 | 18005 | 18024 | 51.1 | 50 | 5753 | 18233 | 18214 | 52 | 50 | 0.9 | 229 | 74.9 | 43.7 | 41 | 52.8 |
| 2234 | 3254 | 3016 | 3036 | 50.2 | 42.9 | 5754 | 3503 | 3484 | 51.5 | 50 | 1.2 | 488 | 76.3 | 43.4 | 41 | 53.6 |
| 2235 | 3255 | 7723 | 7741 | 52.2 | 52.6 | 5755 | 8054 | 8035 | 50.4 | 50 | 1.7 | 332 | 75 | 41.9 | 41 | 52.8 |
| 2236 | 3256 | 29200 | 29224 | 54.2 | 40 | 5756 | 29358 | 29339 | 52.8 | 50 | 1.4 | 159 | 74.5 | 45.9 | 41 | 53.1 |
| 2237 | 3257 | 3016 | 3036 | 50.2 | 42.9 | 5757 | 3504 | 3485 | 50.4 | 45 | 0.1 | 489 | 76.3 | 43.4 | 41 | 53.6 |
| 2238 | 3258 | 985 | 1004 | 51.1 | 50 | 5758 | 1499 | 1482 | 50.1 | 50 | 1.1 | 515 | 76.5 | 43.7 | 41 | 53.7 |
| 2239 | 3259 | 8866 | 8885 | 51.1 | 45 | 5759 | 9257 | 9238 | 50.5 | 45 | 0.6 | 392 | 75 | 41.1 | 41 | 52.8 |
| 2240 | 3260 | 3016 | 3036 | 50.2 | 42.9 | 5760 | 3647 | 3628 | 50.6 | 45 | 0.4 | 632 | 76.4 | 42.9 | 41 | 53.7 |
| 2241 | 3261 | 13039 | 13058 | 51.8 | 50 | 5761 | 13749 | 13727 | 50.5 | 43.5 | 1.3 | 711 | 76.7 | 43.2 | 41 | 53.9 |
| 2242 | 3262 | 24096 | 24119 | 54.4 | 41.7 | 5762 | 24815 | 24792 | 53.4 | 41.7 | 1 | 720 | 75.8 | 41 | 41 | 54.2 |
| 2243 | 3263 | 17840 | 17859 | 50.8 | 45 | 5763 | 18229 | 18209 | 50.1 | 42.9 | 0.7 | 390 | 74.7 | 40.3 | 41 | 52.4 |
| 2244 | 3264 | 15255 | 15273 | 50.3 | 52.6 | 5764 | 15647 | 15628 | 51 | 45 | 0.7 | 393 | 75.1 | 41.2 | 41 | 52.7 |
| 2245 | 3265 | 988 | 1006 | 52.2 | 52.6 | 5765 | 1500 | 1482 | 50.6 | 47.4 | 1.6 | 513 | 76.5 | 43.7 | 41 | 53.8 |
| 2246 | 3266 | 24035 | 24053 | 52.2 | 52.6 | 5766 | 24527 | 24508 | 50.5 | 45 | 1.7 | 493 | 75.4 | 41.2 | 41 | 53 |
| 2247 | 3267 | 18616 | 18636 | 51.4 | 47.6 | 5767 | 19215 | 19194 | 50.2 | 40.9 | 1.1 | 600 | 75.7 | 41.2 | 41 | 53.1 |
| 2248 | 3268 | 8374 | 8393 | 51.2 | 45 | 5768 | 9101 | 9081 | 50.5 | 47.6 | 0.7 | 728 | 75.5 | 40.2 | 41 | 53.1 |
| 2249 | 3269 | 17840 | 17859 | 50.8 | 45 | 5769 | 18238 | 18219 | 50.3 | 45 | 0.5 | 399 | 75 | 40.9 | 41 | 52.7 |
| 2250 | 3270 | 24030 | 24047 | 50.7 | 50 | 5770 | 24526 | 24506 | 50.3 | 42.9 | 0.4 | 497 | 75.5 | 41.2 | 41 | 53 |
| 2251 | 3271 | 24030 | 24047 | 50.7 | 50 | 5771 | 24527 | 24507 | 51 | 42.9 | 0.3 | 498 | 75.4 | 41.2 | 41 | 53.1 |
| 2252 | 3272 | 17840 | 17859 | 50.8 | 45 | 5772 | 18239 | 18220 | 50 | 45 | 0.8 | 400 | 74.9 | 40.8 | 41 | 52.6 |
| 2253 | 3273 | 985 | 1008 | 56.1 | 50 | 5773 | 1626 | 1602 | 56.1 | 44 | 0 | 642 | 77.1 | 44.5 | 41 | 55.9 |
| 2254 | 3274 | 13039 | 13057 | 51.1 | 52.6 | 5774 | 13749 | 13727 | 50.5 | 43.5 | 0.6 | 711 | 76.7 | 43.2 | 41 | 53.9 |
| 2255 | 3275 | 29200 | 29223 | 53.7 | 41.7 | 5775 | 29358 | 29339 | 52.8 | 50 | 0.9 | 159 | 74.5 | 45.9 | 41 | 53.1 |
| 2256 | 3276 | 1046 | 1063 | 50.3 | 50 | 5776 | 1498 | 1481 | 51 | 50 | 0.7 | 453 | 76.4 | 43.9 | 41 | 53.7 |
| 2257 | 3277 | 24019 | 24039 | 50.1 | 42.9 | 5777 | 24527 | 24508 | 50.5 | 45 | 0.4 | 509 | 75.4 | 41.1 | 41 | 52.9 |
| 2258 | 3278 | 24014 | 24035 | 50.6 | 40.9 | 5778 | 24527 | 24508 | 50.5 | 45 | 0.1 | 514 | 75.5 | 41.2 | 41 | 53.1 |
| 2259 | 3279 | 1046 | 1063 | 50.3 | 50 | 5779 | 1497 | 1480 | 50.3 | 50 | 0.1 | 452 | 76.5 | 44 | 41 | 53.7 |
| 2260 | 3280 | 29201 | 29222 | 51 | 40.9 | 5780 | 29358 | 29339 | 52.8 | 50 | 1.9 | 158 | 74.3 | 45.6 | 41 | 52.4 |
| 2261 | 3281 | 18583 | 18603 | 54.8 | 47.6 | 5781 | 18696 | 18672 | 53.9 | 40 | 0.8 | 114 | 70.5 | 40.4 | 41 | 50.6 |
| 2262 | 3282 | 12977 | 12996 | 50.2 | 40 | 5782 | 13326 | 13306 | 50.7 | 42.9 | 0.4 | 350 | 76 | 44 | 41 | 53.4 |
| 2263 | 3283 | 23843 | 23863 | 50.3 | 42.9 | 5783 | 24088 | 24070 | 50.5 | 52.6 | 0.2 | 246 | 75.7 | 45.1 | 41 | 53.2 |
| 2264 | 3284 | 29200 | 29221 | 52.6 | 45.5 | 5784 | 29358 | 29339 | 52.8 | 50 | 0.2 | 159 | 74.5 | 45.9 | 41 | 53 |
| 2265 | 3285 | 23843 | 23863 | 50.3 | 42.9 | 5785 | 24091 | 24073 | 50.9 | 52.6 | 0.5 | 249 | 75.8 | 45.4 | 41 | 53.3 |
| 2266 | 3286 | 17792 | 17813 | 51.6 | 40.9 | 5786 | 18231 | 18210 | 52.2 | 45.5 | 0.6 | 440 | 75 | 40.5 | 41 | 53.1 |
| 2267 | 3287 | 23843 | 23863 | 50.3 | 42.9 | 5787 | 24094 | 24076 | 50.9 | 52.6 | 0.5 | 252 | 75.9 | 45.6 | 41 | 53.4 |
| 2268 | 3288 | 8374 | 8393 | 51.2 | 45 | 5788 | 9109 | 9087 | 50.5 | 43.5 | 0.6 | 736 | 75.4 | 40.1 | 41 | 53.1 |
| 2269 | 3289 | 17793 | 17813 | 50 | 42.9 | 5789 | 18223 | 18206 | 51.8 | 50 | 1.7 | 431 | 74.9 | 40.4 | 41 | 52.5 |
| 2270 | 3290 | 1046 | 1063 | 50.3 | 50 | 5790 | 1481 | 1463 | 50.5 | 47.4 | 0.2 | 436 | 76.2 | 43.6 | 41 | 53.6 |
| 2271 | 3291 | 23842 | 23862 | 50.9 | 47.6 | 5791 | 24093 | 24075 | 50.9 | 52.6 | 0 | 252 | 75.9 | 45.6 | 41 | 53.5 |
| 2272 | 3292 | 23842 | 23862 | 50.9 | 47.6 | 5792 | 24527 | 24507 | 51 | 42.9 | 0.1 | 686 | 76.1 | 41.8 | 41 | 53.6 |
| 2273 | 3293 | 2823 | 2844 | 50.4 | 45.5 | 5793 | 3082 | 3058 | 52.3 | 40 | 1.9 | 260 | 74.3 | 41.5 | 41 | 52.3 |
| 2274 | 3294 | 18550 | 18571 | 50.4 | 40.9 | 5794 | 19316 | 19295 | 50 | 40.9 | 0.4 | 767 | 75.5 | 40.3 | 41 | 53 |
| 2275 | 3295 | 23841 | 23860 | 52.1 | 55 | 5795 | 24527 | 24507 | 51 | 42.9 | 1.1 | 687 | 76.1 | 41.9 | 41 | 53.7 |
| 2276 | 3296 | 23841 | 23860 | 52.1 | 55 | 5796 | 24527 | 24508 | 50.5 | 45 | 1.6 | 687 | 76.1 | 41.9 | 41 | 53.5 |
| 2277 | 3297 | 17793 | 17813 | 50 | 42.9 | 5797 | 18233 | 18215 | 51.3 | 52.6 | 1.3 | 441 | 75.1 | 40.8 | 41 | 52.7 |
| 2278 | 3298 | 1 | 19 | 50.1 | 52.6 | 5798 | 269 | 251 | 51.1 | 52.6 | 1.1 | 269 | 76.4 | 46.5 | 41 | 53.6 |
| 2279 | 3299 | 23841 | 23859 | 50.5 | 52.6 | 5799 | 24094 | 24076 | 50.9 | 52.6 | 0.4 | 254 | 76.1 | 46.1 | 41 | 53.5 |
| 2280 | 3300 | 8908 | 8925 | 51.1 | 50 | 5800 | 9249 | 9231 | 50.8 | 47.4 | 0.2 | 342 | 75.1 | 41.8 | 41 | 52.9 |
| 2281 | 3301 | 8908 | 8925 | 51.1 | 50 | 5801 | 9249 | 9230 | 51.5 | 45 | 0.5 | 342 | 75.1 | 41.8 | 41 | 53 |
| 2282 | 3302 | 23841 | 23859 | 50.5 | 52.6 | 5802 | 24500 | 24481 | 50.1 | 45 | 0.4 | 660 | 76.1 | 42.1 | 41 | 53.4 |
| 2283 | 3303 | 23841 | 23859 | 50.5 | 52.6 | 5803 | 24526 | 24506 | 50.3 | 42.9 | 0.2 | 686 | 76.1 | 42 | 41 | 53.5 |
| 2284 | 3304 | 18225 | 18243 | 51.4 | 52.6 | 5804 | 18632 | 18611 | 50.2 | 40.9 | 1.2 | 408 | 75.7 | 42.6 | 41 | 53.2 |
| 2285 | 3305 | 3794 | 3812 | 52.9 | 52.6 | 5805 | 4318 | 4294 | 54.4 | 40 | 1.5 | 525 | 75.5 | 41.1 | 41 | 53.8 |
| 2286 | 3306 | 8908 | 8925 | 51.1 | 50 | 5806 | 9245 | 9226 | 50 | 45 | 1 | 338 | 74.9 | 41.4 | 41 | 52.5 |
| 2287 | 3307 | 17790 | 17811 | 51.6 | 40.9 | 5807 | 18231 | 18210 | 52.2 | 45.5 | 0.6 | 442 | 75 | 40.5 | 41 | 53.1 |
| 2288 | 3308 | 18077 | 18100 | 54.7 | 45.8 | 5808 | 18443 | 18424 | 55.9 | 55 | 1.3 | 367 | 75.8 | 43.3 | 41 | 54.6 |
| 2289 | 3309 | 23838 | 23857 | 50.4 | 50 | 5809 | 24527 | 24507 | 51 | 42.9 | 0.6 | 690 | 76 | 41.7 | 41 | 53.4 |
| 2290 | 3310 | 23838 | 23857 | 50.4 | 50 | 5810 | 24527 | 24508 | 50.5 | 45 | 0.1 | 690 | 76 | 41.7 | 41 | 53.4 |
| 2291 | 3311 | 23735 | 23752 | 51.2 | 50 | 5811 | 24013 | 23995 | 50.3 | 47.4 | 0.8 | 279 | 74.1 | 40.5 | 41 | 52.1 |
| 2292 | 3312 | 18080 | 18100 | 53.3 | 47.6 | 5812 | 18220 | 18202 | 54.8 | 52.6 | 1.5 | 141 | 73.1 | 44 | 41 | 52.3 |
| 2293 | 3313 | 18081 | 18100 | 51.7 | 50 | 5813 | 18223 | 18206 | 51.8 | 50 | 0.1 | 143 | 73.2 | 44.1 | 41 | 51.9 |
| 2294 | 3314 | 18081 | 18100 | 51.7 | 50 | 5814 | 18231 | 18210 | 52.2 | 45.5 | 0.5 | 151 | 73.6 | 44.4 | 41 | 52.1 |
| 2295 | 3315 | 18081 | 18100 | 51.7 | 50 | 5815 | 18233 | 18214 | 52 | 50 | 0.4 | 153 | 74 | 45.1 | 41 | 52.4 |
| 2296 | 3316 | 18081 | 18100 | 51.7 | 50 | 5816 | 18233 | 18215 | 51.3 | 52.6 | 0.4 | 153 | 74 | 45.1 | 41 | 52.3 |
| 2297 | 3317 | 8911 | 8928 | 51.9 | 50 | 5817 | 9252 | 9235 | 50.1 | 50 | 1.8 | 342 | 75 | 41.5 | 41 | 52.6 |
| 2298 | 3318 | 17791 | 17811 | 50 | 42.9 | 5818 | 18223 | 18206 | 51.8 | 50 | 1.7 | 433 | 74.9 | 40.4 | 41 | 52.5 |
| 2299 | 3319 | 8911 | 8928 | 51.9 | 50 | 5819 | 9249 | 9231 | 50.8 | 47.4 | 1 | 339 | 75 | 41.6 | 41 | 52.8 |
| 2300 | 3320 | 12352 | 12375 | 52.9 | 41.7 | 5820 | 12912 | 12892 | 53.6 | 52.4 | 0.8 | 561 | 76.2 | 42.6 | 41 | 54.3 |
| 2301 | 3321 | 8911 | 8928 | 51.9 | 50 | 5821 | 9249 | 9230 | 51.5 | 45 | 0.3 | 339 | 75 | 41.6 | 41 | 53 |
| 2302 | 3322 | 12352 | 12375 | 52.9 | 41.7 | 5822 | 12995 | 12976 | 51.1 | 45 | 1.8 | 644 | 76.4 | 42.9 | 41 | 53.9 |
| 2303 | 3323 | 17791 | 17811 | 50 | 42.9 | 5823 | 18233 | 18215 | 51.3 | 52.6 | 1.3 | 443 | 75.1 | 40.9 | 41 | 52.7 |
| 2304 | 3324 | 12977 | 12996 | 50.2 | 40 | 5824 | 13328 | 13307 | 51.2 | 45.5 | 1 | 352 | 76 | 44 | 41 | 53.4 |
| 2305 | 3325 | 12977 | 12996 | 50.2 | 40 | 5825 | 13329 | 13308 | 50.5 | 40.9 | 0.3 | 353 | 76 | 43.9 | 41 | 53.4 |
| 2306 | 3326 | 8911 | 8928 | 51.9 | 50 | 5826 | 9245 | 9226 | 50 | 45 | 1.8 | 335 | 74.8 | 41.2 | 41 | 52.5 |
| 2307 | 3327 | 8913 | 8931 | 55.5 | 52.6 | 5827 | 9252 | 9231 | 54 | 45.5 | 1.5 | 340 | 74.9 | 41.5 | 41 | 53.7 |
| 2308 | 3328 | 1402 | 1425 | 52.8 | 41.7 | 5828 | 1501 | 1480 | 51.9 | 40.9 | 0.9 | 100 | 72 | 46 | 41 | 51.1 |
| 2309 | 3329 | 24941 | 24960 | 52 | 50 | 5829 | 25646 | 25627 | 50.5 | 45 | 1.5 | 706 | 75.4 | 40.2 | 41 | 53 |
| 2310 | 3330 | 17608 | 17628 | 50.9 | 42.9 | 5830 | 17769 | 17749 | 50 | 42.9 | 0.9 | 162 | 72.4 | 40.7 | 41 | 50.8 |
| 2311 | 3331 | 24941 | 24960 | 52 | 50 | 5831 | 25404 | 25386 | 52.7 | 52.6 | 0.7 | 464 | 75.3 | 41.2 | 41 | 53.4 |
| 2312 | 3332 | 24941 | 24960 | 52 | 50 | 5832 | 25401 | 25383 | 50.6 | 47.4 | 1.4 | 461 | 75.2 | 41 | 41 | 53 |
| 2313 | 3333 | 24941 | 24960 | 52 | 50 | 5833 | 25400 | 25382 | 51.4 | 52.6 | 0.6 | 460 | 75.3 | 41.1 | 41 | 53.2 |
| 2314 | 3334 | 17608 | 17628 | 50.9 | 42.9 | 5834 | 18231 | 18210 | 52.2 | 45.5 | 1.2 | 624 | 75.2 | 40.1 | 41 | 53.1 |
| 2315 | 3335 | 18081 | 18099 | 51.2 | 52.6 | 5835 | 18232 | 18212 | 50.6 | 47.6 | 0.6 | 152 | 73.8 | 44.7 | 41 | 51.9 |
| 2316 | 3336 | 7725 | 7742 | 50 | 50 | 5836 | 7853 | 7833 | 50.7 | 47.6 | 0.7 | 129 | 71.2 | 40.3 | 41 | 49.9 |
| 2317 | 3337 | 8913 | 8931 | 55.5 | 52.6 | 5837 | 9252 | 9230 | 54.5 | 43.5 | 1 | 340 | 74.9 | 41.5 | 41 | 53.9 |
| 2318 | 3338 | 17608 | 17628 | 50.9 | 42.9 | 5838 | 18233 | 18215 | 51.3 | 52.6 | 0.4 | 626 | 75.3 | 40.3 | 41 | 53.1 |
| 2319 | 3339 | 19715 | 19735 | 52.5 | 47.6 | 5839 | 19931 | 19912 | 50.9 | 55 | 1.6 | 217 | 74 | 41.9 | 41 | 52.2 |
| 2320 | 3340 | 8913 | 8931 | 55.5 | 52.6 | 5840 | 9248 | 9226 | 54.7 | 47.8 | 0.7 | 336 | 74.9 | 41.4 | 41 | 53.9 |
| 2321 | 3341 | 18081 | 18099 | 51.2 | 52.6 | 5841 | 18642 | 18622 | 50.5 | 42.9 | 0.7 | 562 | 76.2 | 42.7 | 41 | 53.6 |
| 2322 | 3342 | 2823 | 2844 | 50.4 | 45.5 | 5842 | 3189 | 3168 | 51 | 45.5 | 0.5 | 367 | 75.6 | 42.8 | 41 | 53.2 |
| 2323 | 3343 | 19715 | 19735 | 52.5 | 47.6 | 5843 | 19927 | 19908 | 52.1 | 55 | 0.3 | 213 | 73.9 | 41.8 | 41 | 52.4 |
| 2324 | 3344 | 2823 | 2844 | 50.4 | 45.5 | 5844 | 3190 | 3169 | 50.7 | 45.5 | 0.2 | 368 | 75.6 | 42.7 | 41 | 53.1 |
| 2325 | 3345 | 28936 | 28956 | 55.2 | 52.4 | 5845 | 29306 | 29287 | 54.6 | 55 | 0.6 | 371 | 76.6 | 45.3 | 41 | 55.1 |
| 2326 | 3346 | 28936 | 28956 | 55.2 | 52.4 | 5846 | 29306 | 29285 | 56.7 | 54.5 | 1.6 | 371 | 76.6 | 45.3 | 41 | 55.3 |
| 2327 | 3347 | 28523 | 28544 | 51.6 | 40.9 | 5847 | 29298 | 29280 | 51.4 | 52.6 | 0.2 | 776 | 78.4 | 47.3 | 41 | 55.4 |
| 2328 | 3348 | 24180 | 24199 | 50.3 | 40 | 5848 | 24933 | 24913 | 51.1 | 42.9 | 0.9 | 754 | 75.8 | 40.8 | 41 | 53.2 |
| 2329 | 3349 | 19715 | 19735 | 52.5 | 47.6 | 5849 | 19925 | 19905 | 51.4 | 52.4 | 1.1 | 211 | 73.8 | 41.7 | 41 | 52.2 |
| 2330 | 3350 | 4645 | 4665 | 50.2 | 42.9 | 5850 | 4836 | 4817 | 51.2 | 45 | 0.9 | 192 | 75 | 45.3 | 41 | 52.6 |
| 2331 | 3351 | 18081 | 18099 | 51.2 | 52.6 | 5851 | 18702 | 18685 | 50.2 | 50 | 1 | 622 | 76.2 | 42.4 | 41 | 53.5 |
| 2332 | 3352 | 28522 | 28542 | 50.2 | 42.9 | 5852 | 29298 | 29280 | 51.4 | 52.6 | 1.2 | 777 | 78.4 | 47.2 | 41 | 55.1 |
| 2333 | 3353 | 24179 | 24199 | 52.7 | 42.9 | 5853 | 24740 | 24717 | 52.5 | 41.7 | 0.2 | 562 | 76 | 42.2 | 41 | 54 |
| 2334 | 3354 | 19715 | 19735 | 52.5 | 47.6 | 5854 | 19909 | 19885 | 52.5 | 40 | 0 | 195 | 73.3 | 41 | 41 | 52.1 |
| 2335 | 3355 | 1810 | 1830 | 51.2 | 42.9 | 5855 | 2103 | 2082 | 52 | 45.5 | 0.8 | 294 | 75.5 | 43.5 | 41 | 53.3 |
| 2336 | 3356 | 19716 | 19737 | 52.2 | 45.5 | 5856 | 19909 | 19885 | 52.5 | 40 | 0.3 | 194 | 73.1 | 40.7 | 41 | 51.9 |
| 2337 | 3357 | 19719 | 19739 | 50.6 | 42.9 | 5857 | 19909 | 19885 | 52.5 | 40 | 1.9 | 191 | 72.9 | 40.3 | 41 | 51.3 |
| 2338 | 3358 | 12977 | 12996 | 50.2 | 40 | 5858 | 13332 | 13312 | 50.9 | 47.6 | 0.6 | 356 | 76.1 | 44.1 | 41 | 53.4 |
| 2339 | 3359 | 19721 | 19745 | 52.3 | 40 | 5859 | 19909 | 19885 | 52.5 | 40 | 0.2 | 189 | 73 | 40.7 | 41 | 51.9 |
| 2340 | 3360 | 17608 | 17627 | 50.2 | 45 | 5860 | 17769 | 17749 | 50 | 42.9 | 0.2 | 162 | 72.4 | 40.7 | 41 | 50.8 |
| 2341 | 3361 | 17608 | 17627 | 50.2 | 45 | 5861 | 18231 | 18210 | 52.2 | 45.5 | 2 | 624 | 75.2 | 40.1 | 41 | 52.8 |
| 2342 | 3362 | 19794 | 19813 | 50 | 50 | 5862 | 20099 | 20078 | 50.5 | 40.9 | 0.5 | 306 | 74.4 | 40.8 | 41 | 52.2 |
| 2343 | 3363 | 4658 | 4677 | 50.5 | 50 | 5863 | 5306 | 5289 | 50.8 | 50 | 0.3 | 649 | 75.5 | 40.7 | 41 | 53.1 |
| 2344 | 3364 | 24179 | 24200 | 53.3 | 40.9 | 5864 | 24580 | 24560 | 51.3 | 52.4 | 2 | 402 | 74.6 | 40 | 41 | 52.7 |
| 2345 | 3365 | 19794 | 19813 | 50 | 50 | 5865 | 19925 | 19906 | 50.1 | 50 | 0.1 | 132 | 72.8 | 43.9 | 41 | 51.1 |
| 2346 | 3366 | 1046 | 1064 | 51.2 | 47.4 | 5866 | 1498 | 1481 | 51 | 50 | 0.2 | 453 | 76.4 | 43.9 | 41 | 53.9 |
| 2347 | 3367 | 1046 | 1064 | 51.2 | 47.4 | 5867 | 1497 | 1480 | 50.3 | 50 | 0.9 | 452 | 76.5 | 44 | 41 | 53.7 |
| 2348 | 3368 | 2133 | 2152 | 50.7 | 45 | 5868 | 2675 | 2656 | 50.4 | 50 | 0.3 | 543 | 76.8 | 44.2 | 41 | 54 |
| 2349 | 3369 | 28965 | 28984 | 52.9 | 55 | 5869 | 29298 | 29279 | 52.6 | 55 | 0.3 | 334 | 76.4 | 45.2 | 41 | 54.4 |
| 2350 | 3370 | 24378 | 24397 | 55 | 55 | 5870 | 24564 | 24542 | 55 | 47.8 | 0 | 187 | 73.6 | 42.2 | 41 | 53.1 |
| 2351 | 3371 | 25348 | 25366 | 51.2 | 47.4 | 5871 | 25651 | 25632 | 52.7 | 50 | 1.6 | 304 | 74.7 | 41.4 | 41 | 52.7 |
| 2352 | 3372 | 19794 | 19813 | 50 | 50 | 5872 | 19923 | 19903 | 50.9 | 47.6 | 0.9 | 130 | 72.7 | 43.8 | 41 | 51 |
| 2353 | 3373 | 28967 | 28987 | 51.6 | 52.4 | 5873 | 29358 | 29339 | 52.8 | 50 | 1.2 | 392 | 76.5 | 44.6 | 41 | 54.1 |
| 2354 | 3374 | 17608 | 17627 | 50.2 | 45 | 5874 | 18233 | 18215 | 51.3 | 52.6 | 1.1 | 626 | 75.3 | 40.3 | 41 | 52.9 |
| 2355 | 3375 | 29186 | 29206 | 51.3 | 42.9 | 5875 | 29358 | 29339 | 52.8 | 50 | 1.5 | 173 | 74.5 | 45.1 | 41 | 52.6 |
| 2356 | 3376 | 24379 | 24398 | 55 | 55 | 5876 | 25093 | 25074 | 54.6 | 55 | 0.4 | 715 | 75.9 | 41.4 | 41 | 54.6 |
| 2357 | 3377 | 3170 | 3191 | 50.9 | 45.5 | 5877 | 3646 | 3625 | 52 | 40.9 | 1.1 | 477 | 75.7 | 41.9 | 41 | 53.4 |
| 2358 | 3378 | 3170 | 3191 | 50.9 | 45.5 | 5878 | 3647 | 3628 | 50.6 | 45 | 0.3 | 478 | 75.7 | 42.1 | 41 | 53.3 |
| 2359 | 3379 | 9140 | 9159 | 50.1 | 45 | 5879 | 9249 | 9230 | 51.5 | 45 | 1.4 | 110 | 71.3 | 42.7 | 41 | 50 |
| 2360 | 3380 | 12976 | 12995 | 51.1 | 45 | 5880 | 13326 | 13306 | 50.7 | 42.9 | 0.4 | 351 | 76.1 | 44.2 | 41 | 53.6 |
| 2361 | 3381 | 12976 | 12995 | 51.1 | 45 | 5881 | 13328 | 13307 | 51.2 | 45.5 | 0.1 | 353 | 76.1 | 44.2 | 41 | 53.7 |
| 2362 | 3382 | 3168 | 3189 | 51 | 45.5 | 5882 | 3494 | 3473 | 50.4 | 40.9 | 0.5 | 327 | 75.1 | 42.2 | 41 | 52.8 |
| 2363 | 3383 | 19794 | 19813 | 50 | 50 | 5883 | 19917 | 19896 | 50.9 | 45.5 | 0.9 | 124 | 72.3 | 43.5 | 41 | 50.7 |
| 2364 | 3384 | 24379 | 24398 | 55 | 55 | 5884 | 24517 | 24494 | 53.2 | 41.7 | 1.8 | 139 | 72.7 | 43.2 | 41 | 52 |
| 2365 | 3385 | 24380 | 24399 | 55 | 55 | 5885 | 25093 | 25074 | 54.6 | 55 | 0.4 | 714 | 76 | 41.5 | 41 | 54.6 |
| 2366 | 3386 | 2823 | 2844 | 50.4 | 45.5 | 5886 | 3201 | 3183 | 50.6 | 52.6 | 0.2 | 379 | 75.8 | 43 | 41 | 53.3 |
| 2367 | 3387 | 3798 | 3819 | 54.2 | 50 | 5887 | 4318 | 4294 | 54.4 | 40 | 0.2 | 521 | 75.4 | 41.1 | 41 | 54.2 |
| 2368 | 3388 | 9139 | 9159 | 52.5 | 47.6 | 5888 | 9852 | 9829 | 53.1 | 45.8 | 0.6 | 714 | 75.4 | 40.1 | 41 | 53.6 |
| 2369 | 3389 | 9139 | 9159 | 52.5 | 47.6 | 5889 | 9852 | 9828 | 53.6 | 44 | 1.1 | 714 | 75.4 | 40.1 | 41 | 53.6 |
| 2370 | 3390 | 19795 | 19814 | 50.4 | 45 | 5890 | 19927 | 19908 | 52.1 | 55 | 1.7 | 133 | 72.7 | 43.6 | 41 | 51.1 |
| 2371 | 3391 | 19795 | 19814 | 50.4 | 45 | 5891 | 19924 | 19905 | 50.1 | 50 | 0.3 | 130 | 72.4 | 43.1 | 41 | 50.8 |
| 2372 | 3392 | 12976 | 12995 | 51.1 | 45 | 5892 | 13329 | 13308 | 50.5 | 40.9 | 0.6 | 354 | 76.1 | 44.1 | 41 | 53.5 |
| 2373 | 3393 | 2133 | 2152 | 50.7 | 45 | 5893 | 2672 | 2654 | 50.9 | 52.6 | 0.2 | 540 | 76.8 | 44.3 | 41 | 54.1 |
| 2374 | 3394 | 4593 | 4613 | 51.5 | 47.6 | 5894 | 4836 | 4817 | 51.2 | 45 | 0.3 | 244 | 75.3 | 44.3 | 41 | 53.2 |
| 2375 | 3395 | 1810 | 1830 | 51.2 | 42.9 | 5895 | 2113 | 2094 | 50.1 | 45 | 1.1 | 304 | 75.5 | 43.4 | 41 | 53 |
| 2376 | 3396 | 17036 | 17058 | 53.5 | 47.8 | 5896 | 17483 | 17465 | 54.4 | 52.6 | 0.9 | 448 | 75.3 | 41.3 | 41 | 53.9 |
| 2377 | 3397 | 1046 | 1064 | 51.2 | 47.4 | 5897 | 1481 | 1463 | 50.5 | 47.4 | 0.6 | 436 | 76.2 | 43.6 | 41 | 53.6 |
| 2378 | 3398 | 9055 | 9079 | 52.8 | 40 | 5898 | 9255 | 9236 | 51.1 | 45 | 1.8 | 201 | 73.5 | 41.3 | 41 | 51.9 |
| 2379 | 3399 | 12976 | 12995 | 51.1 | 45 | 5899 | 13332 | 13312 | 50.9 | 47.6 | 0.2 | 357 | 76.2 | 44.3 | 41 | 53.7 |
| 2380 | 3400 | 8865 | 8884 | 50.4 | 45 | 5900 | 9311 | 9292 | 50.7 | 50 | 0.3 | 447 | 75.4 | 41.4 | 41 | 53 |
| 2381 | 3401 | 25363 | 25381 | 51.1 | 52.6 | 5901 | 25651 | 25632 | 52.7 | 50 | 1.6 | 289 | 74.3 | 40.8 | 41 | 52.5 |
| 2382 | 3402 | 3168 | 3189 | 51 | 45.5 | 5902 | 3504 | 3485 | 50.4 | 45 | 0.6 | 337 | 75.3 | 42.4 | 41 | 52.9 |
| 2383 | 3403 | 25363 | 25381 | 51.1 | 52.6 | 5903 | 25649 | 25629 | 51.5 | 42.9 | 0.3 | 287 | 74.1 | 40.4 | 41 | 52.3 |
| 2384 | 3404 | 29182 | 29202 | 51.2 | 42.9 | 5904 | 29414 | 29395 | 50.5 | 50 | 0.7 | 233 | 75.5 | 45.1 | 41 | 53.1 |
| 2385 | 3405 | 3031 | 3051 | 51.3 | 52.4 | 5905 | 3497 | 3478 | 51.3 | 50 | 0.1 | 467 | 76.3 | 43.5 | 41 | 53.9 |
| 2386 | 3406 | 29172 | 29192 | 51.5 | 42.9 | 5906 | 29412 | 29393 | 50.3 | 45 | 1.1 | 241 | 75.6 | 45.2 | 41 | 53.2 |
| 2387 | 3407 | 12040 | 12057 | 50.6 | 50 | 5907 | 12412 | 12392 | 50 | 42.9 | 0.6 | 373 | 75.9 | 43.4 | 41 | 53.2 |
| 2388 | 3408 | 11543 | 11562 | 50.4 | 40 | 5908 | 12110 | 12090 | 51.1 | 42.9 | 0.7 | 568 | 75.9 | 41.9 | 41 | 53.4 |
| 2389 | 3409 | 16909 | 16928 | 50.8 | 45 | 5909 | 17038 | 17021 | 50.7 | 50 | 0.1 | 130 | 72.7 | 43.8 | 41 | 51.2 |
| 2390 | 3410 | 16909 | 16928 | 50.8 | 45 | 5910 | 17039 | 17022 | 51.4 | 50 | 0.6 | 131 | 72.6 | 43.5 | 41 | 51.2 |
| 2391 | 3411 | 18077 | 18097 | 51.5 | 47.6 | 5911 | 18233 | 18214 | 52 | 50 | 0.5 | 157 | 73.9 | 44.6 | 41 | 52.3 |
| 2392 | 3412 | 18077 | 18097 | 51.5 | 47.6 | 5912 | 18233 | 18215 | 51.3 | 52.6 | 0.2 | 157 | 73.9 | 44.6 | 41 | 52.2 |
| 2393 | 3413 | 9055 | 9079 | 52.8 | 40 | 5913 | 9252 | 9234 | 51.4 | 52.6 | 1.4 | 198 | 73.7 | 41.9 | 41 | 52.1 |
| 2394 | 3414 | 25676 | 25697 | 51.9 | 40.9 | 5914 | 25832 | 25810 | 53.6 | 47.8 | 1.7 | 157 | 72.1 | 40.1 | 41 | 51.1 |
| 2395 | 3415 | 2223 | 2244 | 51.4 | 45.5 | 5915 | 2676 | 2657 | 50.7 | 50 | 0.7 | 454 | 76.9 | 45.2 | 41 | 54.2 |
| 2396 | 3416 | 619 | 640 | 50.4 | 45.5 | 5916 | 1171 | 1153 | 50.4 | 47.4 | 0 | 553 | 77.9 | 46.8 | 41 | 54.7 |
| 2397 | 3417 | 11541 | 11561 | 50.9 | 42.9 | 5917 | 12110 | 12090 | 51.1 | 42.9 | 0.3 | 570 | 75.9 | 41.9 | 41 | 53.5 |
| 2398 | 3418 | 3360 | 3379 | 50.7 | 45 | 5918 | 3497 | 3478 | 51.3 | 50 | 0.6 | 138 | 74 | 46.4 | 42 | 52.1 |
| 2399 | 3419 | 19725 | 19745 | 50 | 42.9 | 5919 | 19921 | 19901 | 50.2 | 47.6 | 0.1 | 197 | 73.5 | 41.6 | 42 | 51.6 |
| 2400 | 3420 | 19720 | 19740 | 51.3 | 42.9 | 5920 | 19921 | 19901 | 50.2 | 47.6 | 1.1 | 202 | 73.4 | 41.1 | 42 | 51.5 |
| 2401 | 3421 | 3360 | 3379 | 50.7 | 45 | 5921 | 3500 | 3481 | 51.2 | 50 | 0.5 | 141 | 74 | 46.1 | 42 | 52.1 |
| 2402 | 3422 | 19717 | 19738 | 50.8 | 40.9 | 5922 | 19921 | 19901 | 50.2 | 47.6 | 0.6 | 205 | 73.4 | 41 | 42 | 51.5 |
| 2403 | 3423 | 24562 | 24580 | 50.1 | 52.6 | 5923 | 25209 | 25190 | 50.6 | 50 | 0.5 | 648 | 76.1 | 42 | 42 | 53.4 |
| 2404 | 3424 | 24559 | 24579 | 52 | 52.4 | 5924 | 24740 | 24717 | 52.5 | 41.7 | 0.5 | 182 | 76 | 48.4 | 42 | 53.9 |
| 2405 | 3425 | 3360 | 3379 | 50.7 | 45 | 5925 | 3504 | 3485 | 50.4 | 45 | 0.3 | 145 | 74.2 | 46.2 | 42 | 52.2 |
| 2406 | 3426 | 19716 | 19737 | 52.2 | 45.5 | 5926 | 19921 | 19900 | 51.8 | 45.5 | 0.4 | 206 | 73.5 | 41.3 | 42 | 52.1 |
| 2407 | 3427 | 3232 | 3251 | 50.3 | 50 | 5927 | 3494 | 3473 | 50.4 | 40.9 | 0.1 | 263 | 74.3 | 41.4 | 42 | 52.2 |
| 2408 | 3428 | 26039 | 26058 | 54 | 55 | 5928 | 26657 | 26636 | 52.6 | 45.5 | 1.5 | 619 | 75.4 | 40.5 | 42 | 53.7 |
| 2409 | 3429 | 3232 | 3251 | 50.3 | 50 | 5929 | 3504 | 3485 | 50.4 | 45 | 0.1 | 273 | 74.6 | 41.8 | 42 | 52.4 |
| 2410 | 3430 | 19715 | 19735 | 52.5 | 47.6 | 5930 | 19921 | 19900 | 51.8 | 45.5 | 0.7 | 207 | 73.7 | 41.5 | 42 | 52.2 |
| 2411 | 3431 | 26039 | 26058 | 54 | 55 | 5931 | 26653 | 26631 | 53.2 | 43.5 | 0.9 | 615 | 75.3 | 40.3 | 42 | 53.8 |
| 2412 | 3432 | 3229 | 3248 | 50.6 | 50 | 5932 | 3494 | 3473 | 50.4 | 40.9 | 0.2 | 266 | 74.3 | 41.4 | 42 | 52.3 |
| 2413 | 3433 | 3229 | 3248 | 50.6 | 50 | 5933 | 3504 | 3485 | 50.4 | 45 | 0.3 | 276 | 74.5 | 41.7 | 42 | 52.4 |
| 2414 | 3434 | 3225 | 3244 | 52.4 | 55 | 5934 | 3495 | 3473 | 51.8 | 43.5 | 0.6 | 271 | 74.7 | 42.1 | 42 | 52.9 |
| 2415 | 3435 | 3222 | 3241 | 52 | 50 | 5935 | 3650 | 3631 | 53.1 | 50 | 1.1 | 429 | 75.5 | 42 | 42 | 53.6 |
| 2416 | 3436 | 24559 | 24579 | 52 | 52.4 | 5936 | 25209 | 25190 | 50.6 | 50 | 1.3 | 651 | 76.1 | 42.1 | 42 | 53.6 |
| 2417 | 3437 | 6158 | 6178 | 51.3 | 42.9 | 5937 | 6289 | 6267 | 52.2 | 43.5 | 0.9 | 132 | 71.3 | 40.2 | 42 | 50.4 |
| 2418 | 3438 | 19709 | 19730 | 51.3 | 40.9 | 5938 | 19917 | 19896 | 50.9 | 45.5 | 0.3 | 209 | 73.7 | 41.6 | 42 | 52 |
| 2419 | 3439 | 3223 | 3241 | 50.2 | 52.6 | 5939 | 3497 | 3478 | 51.3 | 50 | 1.1 | 275 | 74.7 | 42.2 | 42 | 52.5 |
| 2420 | 3440 | 3223 | 3241 | 50.2 | 52.6 | 5940 | 3646 | 3625 | 52 | 40.9 | 1.8 | 424 | 75.4 | 41.7 | 42 | 53 |
| 2421 | 3441 | 3223 | 3241 | 50.2 | 52.6 | 5941 | 3647 | 3628 | 50.6 | 45 | 0.4 | 425 | 75.5 | 41.9 | 42 | 53 |
| 2422 | 3442 | 3217 | 3237 | 51.8 | 47.6 | 5942 | 3650 | 3631 | 53.1 | 50 | 1.3 | 434 | 75.5 | 41.9 | 42 | 53.5 |
| 2423 | 3443 | 9352 | 9372 | 51.3 | 42.9 | 5943 | 10014 | 9996 | 50.7 | 52.6 | 0.6 | 663 | 75.6 | 40.7 | 42 | 53.2 |
| 2424 | 3444 | 23733 | 23752 | 55.6 | 55 | 5944 | 24022 | 24003 | 55.5 | 55 | 0.1 | 290 | 74.5 | 41.4 | 42 | 53.9 |
| 2425 | 3445 | 26040 | 26061 | 56.4 | 54.5 | 5945 | 26661 | 26639 | 55.3 | 47.8 | 1.2 | 622 | 75.5 | 40.7 | 42 | 54.5 |
| 2426 | 3446 | 9918 | 9938 | 51.4 | 47.6 | 5946 | 10608 | 10589 | 51 | 50 | 0.4 | 691 | 75.8 | 41.1 | 42 | 53.4 |
| 2427 | 3447 | 7724 | 7742 | 51.4 | 52.6 | 5947 | 7843 | 7825 | 52.8 | 52.6 | 1.3 | 120 | 70.7 | 40 | 42 | 50 |
| 2428 | 3448 | 26040 | 26061 | 56.4 | 54.5 | 5948 | 26655 | 26631 | 56.2 | 48 | 0.2 | 616 | 75.4 | 40.6 | 42 | 54.8 |
| 2429 | 3449 | 28117 | 28135 | 50.6 | 52.6 | 5949 | 28506 | 28488 | 50.2 | 47.4 | 0.4 | 390 | 79.4 | 51.8 | 42 | 55.8 |
| 2430 | 3450 | 3217 | 3236 | 51.1 | 50 | 5950 | 3497 | 3478 | 51.3 | 50 | 0.2 | 281 | 74.7 | 42 | 42 | 52.7 |
| 2431 | 3451 | 3217 | 3236 | 51.1 | 50 | 5951 | 3500 | 3481 | 51.2 | 50 | 0.1 | 284 | 74.7 | 41.9 | 42 | 52.7 |
| 2432 | 3452 | 3165 | 3187 | 51.6 | 43.5 | 5952 | 3650 | 3631 | 53.1 | 50 | 1.5 | 486 | 75.8 | 42.2 | 42 | 53.6 |
| 2433 | 3453 | 19709 | 19730 | 51.3 | 40.9 | 5953 | 19925 | 19906 | 50.1 | 50 | 1.2 | 217 | 74 | 41.9 | 42 | 51.9 |
| 2434 | 3454 | 9927 | 9945 | 50.8 | 52.6 | 5954 | 10199 | 10180 | 51.5 | 45 | 0.7 | 273 | 75.3 | 43.6 | 42 | 53.1 |
| 2435 | 3455 | 9929 | 9946 | 50 | 50 | 5955 | 10670 | 10649 | 51.3 | 40.9 | 1.3 | 742 | 75.7 | 40.8 | 42 | 53.1 |
| 2436 | 3456 | 19709 | 19730 | 51.3 | 40.9 | 5956 | 19927 | 19908 | 52.1 | 55 | 0.9 | 219 | 74 | 42 | 42 | 52.3 |
| 2437 | 3457 | 9934 | 9953 | 50.7 | 50 | 5957 | 10356 | 10336 | 52.4 | 47.6 | 1.7 | 423 | 75.6 | 42.1 | 42 | 53.2 |
| 2438 | 3458 | 19709 | 19730 | 51.3 | 40.9 | 5958 | 19930 | 19910 | 50.6 | 47.6 | 0.7 | 222 | 74 | 41.9 | 42 | 52.1 |
| 2439 | 3459 | 3164 | 3186 | 51.6 | 43.5 | 5959 | 3650 | 3631 | 53.1 | 50 | 1.5 | 487 | 75.9 | 42.3 | 42 | 53.7 |
| 2440 | 3460 | 3089 | 3110 | 51.8 | 45.5 | 5960 | 3188 | 3166 | 51.6 | 43.5 | 0.2 | 100 | 72 | 46 | 42 | 51 |
| 2441 | 3461 | 18979 | 19000 | 51.6 | 45.5 | 5961 | 19215 | 19194 | 50.2 | 40.9 | 1.4 | 237 | 73.5 | 40.1 | 42 | 51.6 |
| 2442 | 3462 | 26421 | 26441 | 51.5 | 42.9 | 5962 | 26900 | 26882 | 51.5 | 52.6 | 0.1 | 480 | 77.5 | 46.2 | 42 | 54.8 |
| 2443 | 3463 | 26421 | 26441 | 51.5 | 42.9 | 5963 | 26828 | 26810 | 52.9 | 52.6 | 1.4 | 408 | 76.6 | 44.9 | 42 | 54.2 |
| 2444 | 3464 | 11540 | 11557 | 50.4 | 50 | 5964 | 11826 | 11802 | 51.3 | 40 | 0.8 | 287 | 74.4 | 41.1 | 42 | 52.3 |
| 2445 | 3465 | 26421 | 26441 | 51.5 | 42.9 | 5965 | 26695 | 26678 | 50.5 | 50 | 1 | 275 | 74.9 | 42.5 | 42 | 52.7 |
| 2446 | 3466 | 11540 | 11557 | 50.4 | 50 | 5966 | 11819 | 11798 | 50.3 | 40.9 | 0.1 | 280 | 74.3 | 41.1 | 42 | 52.2 |
| 2447 | 3467 | 11540 | 11557 | 50.4 | 50 | 5967 | 11817 | 11797 | 50.4 | 42.9 | 0.1 | 278 | 74.3 | 41 | 42 | 52.2 |
| 2448 | 3468 | 23841 | 23859 | 50.5 | 52.6 | 5968 | 24515 | 24494 | 50.4 | 40.9 | 0.1 | 675 | 76.1 | 41.9 | 42 | 53.5 |
| 2449 | 3469 | 3055 | 3077 | 52.8 | 43.5 | 5969 | 3495 | 3473 | 51.8 | 43.5 | 0.9 | 441 | 76 | 43.1 | 42 | 53.9 |
| 2450 | 3470 | 3795 | 3813 | 52.1 | 52.6 | 5970 | 4565 | 4542 | 53.9 | 41.7 | 1.8 | 771 | 75.6 | 40.3 | 42 | 53.6 |
| 2451 | 3471 | 11540 | 11560 | 53.2 | 47.6 | 5971 | 11984 | 11966 | 53 | 52.6 | 0.2 | 445 | 75.1 | 40.7 | 42 | 53.6 |
| 2452 | 3472 | 11541 | 11561 | 50.9 | 42.9 | 5972 | 12165 | 12147 | 51.2 | 47.4 | 0.4 | 625 | 75.7 | 41.3 | 42 | 53.4 |
| 2453 | 3473 | 3795 | 3815 | 54.6 | 52.4 | 5973 | 4318 | 4294 | 54.4 | 40 | 0.2 | 524 | 75.5 | 41.2 | 42 | 54.3 |
| 2454 | 3474 | 7723 | 7741 | 52.2 | 52.6 | 5974 | 7853 | 7833 | 50.7 | 47.6 | 1.5 | 131 | 71.3 | 40.5 | 42 | 50.2 |
| 2455 | 3475 | 3055 | 3075 | 51.8 | 47.6 | 5975 | 3504 | 3485 | 50.4 | 45 | 1.4 | 450 | 76.1 | 43.1 | 42 | 53.5 |
| 2456 | 3476 | 3055 | 3074 | 51.1 | 50 | 5976 | 3494 | 3473 | 50.4 | 40.9 | 0.7 | 440 | 76 | 43 | 42 | 53.4 |
| 2457 | 3477 | 26421 | 26441 | 51.5 | 42.9 | 5977 | 26651 | 26631 | 50.2 | 42.9 | 1.3 | 231 | 73.8 | 41.1 | 42 | 51.8 |
| 2458 | 3478 | 28109 | 28130 | 50.2 | 40.9 | 5978 | 28672 | 28654 | 50.6 | 52.6 | 0.4 | 564 | 79.9 | 51.6 | 42 | 56.1 |
| 2459 | 3479 | 3055 | 3074 | 51.1 | 50 | 5979 | 3504 | 3485 | 50.4 | 45 | 0.7 | 450 | 76.1 | 43.1 | 42 | 53.5 |
| 2460 | 3480 | 12232 | 12250 | 51.9 | 52.6 | 5980 | 12412 | 12392 | 50 | 42.9 | 1.9 | 181 | 73.2 | 41.4 | 42 | 51.3 |
| 2461 | 3481 | 3034 | 3053 | 50.3 | 50 | 5981 | 3210 | 3190 | 50.5 | 47.6 | 0.2 | 177 | 74.9 | 45.8 | 42 | 52.6 |
| 2462 | 3482 | 3034 | 3053 | 50.3 | 50 | 5982 | 3494 | 3473 | 50.4 | 40.9 | 0.1 | 461 | 76.1 | 43.2 | 42 | 53.5 |
| 2463 | 3483 | 3034 | 3053 | 50.3 | 50 | 5983 | 3504 | 3485 | 50.4 | 45 | 0.1 | 471 | 76.2 | 43.3 | 42 | 53.5 |
| 2464 | 3484 | 12236 | 12256 | 51.2 | 42.9 | 5984 | 12498 | 12480 | 50 | 47.4 | 1.1 | 263 | 74.6 | 42.2 | 42 | 52.4 |
| 2465 | 3485 | 12352 | 12375 | 52.9 | 41.7 | 5985 | 12724 | 12705 | 52.4 | 55 | 0.5 | 373 | 75.7 | 42.9 | 42 | 53.8 |
| 2466 | 3486 | 26421 | 26441 | 51.5 | 42.9 | 5986 | 26585 | 26567 | 51 | 47.4 | 0.5 | 165 | 72.2 | 40 | 42 | 50.9 |
| 2467 | 3487 | 3031 | 3051 | 51.3 | 52.4 | 5987 | 3503 | 3484 | 51.5 | 50 | 0.1 | 473 | 76.3 | 43.6 | 42 | 53.9 |
| 2468 | 3488 | 18704 | 18724 | 50.8 | 47.6 | 5988 | 19480 | 19459 | 50.3 | 40.9 | 0.4 | 777 | 75.5 | 40.2 | 42 | 53.1 |
| 2469 | 3489 | 3016 | 3036 | 50.2 | 42.9 | 5989 | 3646 | 3625 | 52 | 40.9 | 1.8 | 631 | 76.4 | 42.8 | 42 | 53.6 |
| 2470 | 3490 | 2823 | 2844 | 50.4 | 45.5 | 5990 | 3053 | 3034 | 50.3 | 50 | 0.2 | 231 | 74 | 41.6 | 42 | 52 |
| 2471 | 3491 | 12366 | 12384 | 51.7 | 52.6 | 5991 | 12994 | 12976 | 50.3 | 47.4 | 1.3 | 629 | 76.4 | 42.9 | 42 | 53.7 |
| 2472 | 3492 | 12366 | 12384 | 51.7 | 52.6 | 5992 | 12992 | 12974 | 51.2 | 52.6 | 0.5 | 627 | 76.5 | 43.1 | 42 | 54 |
| 2473 | 3493 | 2823 | 2844 | 50.4 | 45.5 | 5993 | 3056 | 3037 | 52.1 | 55 | 1.6 | 234 | 74.2 | 41.9 | 42 | 52.2 |
| 2474 | 3494 | 2522 | 2541 | 51.4 | 45 | 5994 | 2672 | 2654 | 50.9 | 52.6 | 0.5 | 151 | 75.3 | 48.3 | 42 | 53 |
| 2475 | 3495 | 2522 | 2541 | 51.4 | 45 | 5995 | 2675 | 2656 | 50.4 | 50 | 1 | 154 | 75.2 | 48.1 | 42 | 52.9 |
| 2476 | 3496 | 2429 | 2447 | 50.2 | 47.4 | 5996 | 3056 | 3037 | 52.1 | 55 | 1.9 | 628 | 76.3 | 42.7 | 42 | 53.6 |
| 2477 | 3497 | 2429 | 2447 | 50.2 | 47.4 | 5997 | 3190 | 3169 | 50.7 | 45.5 | 0.5 | 762 | 76.6 | 42.9 | 42 | 53.8 |
| 2478 | 3498 | 27436 | 27455 | 52.7 | 45 | 5998 | 27541 | 27521 | 51.7 | 47.6 | 1 | 106 | 72.1 | 45.3 | 42 | 51.1 |
| 2479 | 3499 | 2429 | 2447 | 50.2 | 47.4 | 5999 | 3192 | 3171 | 51.9 | 50 | 1.7 | 764 | 76.7 | 43.1 | 42 | 53.8 |
| 2480 | 3500 | 2427 | 2445 | 52.1 | 52.6 | 6000 | 3056 | 3037 | 52.1 | 55 | 0 | 630 | 76.4 | 42.9 | 42 | 54.2 |
| 2481 | 3501 | 27389 | 27407 | 50.6 | 47.4 | 6001 | 27541 | 27521 | 51.7 | 47.6 | 1.1 | 153 | 73.2 | 43.1 | 42 | 51.5 |
| 2482 | 3502 | 2427 | 2445 | 52.1 | 52.6 | 6002 | 3190 | 3169 | 50.7 | 45.5 | 1.4 | 764 | 76.7 | 43.1 | 42 | 54 |
| 2483 | 3503 | 18616 | 18636 | 51.4 | 47.6 | 6003 | 19316 | 19295 | 50 | 40.9 | 1.4 | 701 | 75.4 | 40.2 | 42 | 52.9 |
| 2484 | 3504 | 2377 | 2395 | 52.4 | 52.6 | 6004 | 2672 | 2653 | 51.6 | 50 | 0.8 | 296 | 77 | 47.3 | 42 | 54.5 |
| 2485 | 3505 | 18591 | 18611 | 51.7 | 42.9 | 6005 | 19216 | 19195 | 50.2 | 40.9 | 1.4 | 626 | 75.7 | 41.1 | 42 | 53.1 |
| 2486 | 3506 | 12366 | 12384 | 51.7 | 52.6 | 6006 | 12739 | 12718 | 51 | 40.9 | 0.7 | 374 | 75.6 | 42.8 | 42 | 53.3 |
| 2487 | 3507 | 2377 | 2395 | 52.4 | 52.6 | 6007 | 2672 | 2654 | 50.9 | 52.6 | 1.5 | 296 | 77 | 47.3 | 42 | 54.3 |
| 2488 | 3508 | 16982 | 17001 | 51.2 | 55 | 6008 | 17111 | 17090 | 51.1 | 40.9 | 0.1 | 130 | 74.6 | 48.5 | 42 | 52.6 |
| 2489 | 3509 | 2377 | 2395 | 52.4 | 52.6 | 6009 | 2675 | 2656 | 50.4 | 50 | 2 | 299 | 77 | 47.2 | 42 | 54.1 |
| 2490 | 3510 | 18590 | 18608 | 50.6 | 42.1 | 6010 | 19216 | 19195 | 50.2 | 40.9 | 0.3 | 627 | 75.6 | 41 | 42 | 53.1 |
| 2491 | 3511 | 2377 | 2395 | 52.4 | 52.6 | 6011 | 2891 | 2873 | 50.8 | 47.4 | 1.6 | 515 | 76.8 | 44.5 | 42 | 54.1 |
| 2492 | 3512 | 8220 | 8240 | 54 | 47.6 | 6012 | 8935 | 8917 | 54.5 | 52.6 | 0.4 | 716 | 75.4 | 40.1 | 42 | 54.1 |
| 2493 | 3513 | 12370 | 12388 | 50.1 | 47.4 | 6013 | 12998 | 12979 | 50.1 | 45 | 0.1 | 629 | 76.4 | 42.9 | 42 | 53.6 |
| 2494 | 3514 | 2223 | 2244 | 51.4 | 45.5 | 6014 | 2675 | 2656 | 50.4 | 50 | 1 | 453 | 77 | 45.3 | 42 | 54.1 |
| 2495 | 3515 | 2220 | 2239 | 51.3 | 45 | 6015 | 2672 | 2654 | 50.9 | 52.6 | 0.4 | 453 | 77 | 45.3 | 42 | 54.2 |
| 2496 | 3516 | 24418 | 24439 | 52.9 | 45.5 | 6016 | 24936 | 24919 | 51.8 | 50 | 1.1 | 519 | 76 | 42.4 | 42 | 53.8 |
| 2497 | 3517 | 18586 | 18603 | 50.4 | 44.4 | 6017 | 19216 | 19195 | 50.2 | 40.9 | 0.2 | 631 | 75.6 | 40.9 | 42 | 53.1 |
| 2498 | 3518 | 2220 | 2239 | 51.3 | 45 | 6018 | 2675 | 2656 | 50.4 | 50 | 0.8 | 456 | 76.9 | 45.2 | 42 | 54.1 |
| 2499 | 3519 | 1402 | 1422 | 50.2 | 42.9 | 6019 | 2153 | 2134 | 50.4 | 45 | 0.2 | 752 | 76.7 | 43.1 | 42 | 53.8 |
| 2500 | 3520 | 1356 | 1375 | 53.8 | 55 | 6020 | 2153 | 2133 | 52.1 | 42.9 | 1.7 | 798 | 76.9 | 43.5 | 42 | 54.5 |

TABLE 5
Primers

| | | | | T_{M} | | Product |
|---|---|---|---|---|---|---|
| Forward primer SEQ | | Reverse primer SEQ | | (FOR & REV) | | length |
| ID NO & Co-ordinates | | ID NO & Co-ordinates | | (° C.) | | (bp) |
| 6076 | 1-19 | 6171 | 199-183 | 50.1 | 50.3 | 199 |
| 6077 | 149-169 | 6172 | 334-315 | 51.5 | 52.4 | 186 |
| 6078 | 292-310 | 6173 | 560-541 | 50.8 | 51.1 | 269 |
| 6079 | 598-619 | 6174 | 749-731 | 52.6 | 50.6 | 152 |
| 6080 | 721-742 | 6175 | 930-912 | 50.4 | 50.3 | 210 |
| 6081 | 888-912 | 6176 | 1077-1058 | 52.8 | 51.2 | 190 |
| 6082 | 984-1003 | 6177 | 1149-1131 | 51.1 | 51.1 | 166 |
| 6083 | 1157-1175 | 6178 | 1479-1460 | 50.9 | 51.6 | 323 |
| 6084 | 1420-1441 | 6179 | 1700-1680 | 51.2 | 50.7 | 281 |
| 6085 | 1685-1707 | 6180 | 1834-1811 | 53.8 | 53.7 | 150 |
| 6086 | 1740-1764 | 6181 | 1987-1963 | 53.4 | 52.2 | 248 |
| 6087 | 2007-2025 | 6182 | 2251-2232 | 50.3 | 50.1 | 245 |
| 6088 | 2226-2245 | 6183 | 2385-2366 | 50.4 | 50.1 | 160 |
| 6089 | 2428-2446 | 6184 | 2749-2728 | 50.1 | 50.3 | 322 |
| 6090 | 2742-2763 | 6185 | 2893-2875 | 50.6 | 51.4 | 152 |
| 6091 | 2823-2844 | 6186 | 3082-3058 | 50.4 | 52.3 | 260 |
| 6092 | 3007-3031 | 6187 | 3185-3164 | 51.9 | 51.0 | 179 |
| 6093 | 3234-3254 | 6188 | 3497-3478 | 51.1 | 51.3 | 264 |
| 6094 | 3453-3476 | 6189 | 3647-3627 | 51.8 | 52.1 | 195 |
| 6095 | 3601-3622 | 6190 | 3877-3853 | 52.5 | 53.6 | 277 |
| 6096 | 4007-4027 | 6191 | 4158-4135 | 51.1 | 51.4 | 152 |
| 6097 | 4141-4165 | 6192 | 4316-4295 | 51.3 | 50.8 | 176 |
| 6098 | 4366-4387 | 6193 | 4567-4544 | 54.6 | 55.4 | 202 |
| 6099 | 4488-4508 | 6194 | 4708-4690 | 50.7 | 50.3 | 221 |
| 6100 | 4658-4677 | 6195 | 4994-4974 | 50.5 | 51.2 | 337 |
| 6101 | 4902-4922 | 6196 | 5115-5092 | 50.5 | 51.4 | 214 |
| 6102 | 5239-5260 | 6197 | 5450-5430 | 50.8 | 50.9 | 212 |
| 6103 | 5366-5389 | 6198 | 5560-5542 | 50.5 | 51.8 | 195 |
| 6104 | 5593-5612 | 6199 | 5860-5836 | 50.8 | 51.6 | 268 |
| 6105 | 6042-6062 | 6200 | 6291-6271 | 50.4 | 51.1 | 250 |
| 6106 | 6271-6291 | 6201 | 6483-6463 | 51.1 | 50.2 | 213 |
| 6107 | 7017-7040 | 6202 | 7171-7153 | 52.4 | 52.8 | 155 |
| 6108 | 7253-7272 | 6203 | 7504-7486 | 50.3 | 50.3 | 252 |
| 6109 | 7415-7434 | 6204 | 7677-7654 | 54.5 | 53.6 | 263 |
| 6110 | 7615-7635 | 6205 | 7821-7798 | 51.1 | 52.8 | 207 |
| 6111 | 7728-7746 | 6206 | 7936-7915 | 51.7 | 50.1 | 209 |
| 6112 | 7845-7867 | 6207 | 7994-7970 | 52.7 | 53.4 | 150 |
| 6113 | 8011-8029 | 6208 | 8189-8170 | 51.4 | 50.6 | 179 |
| 6114 | 8143-8166 | 6209 | 8300-8281 | 52.2 | 50.8 | 158 |
| 6115 | 8221-8239 | 6210 | 8388-8369 | 51.0 | 51.1 | 168 |
| 6116 | 8553-8575 | 6211 | 8931-8915 | 51.8 | 50.3 | 379 |
| 6117 | 8867-8886 | 6212 | 9254-9236 | 50.7 | 50.6 | 388 |
| 6118 | 9244-9267 | 6213 | 9597-9573 | 51.9 | 53.4 | 354 |
| 6119 | 9620-9640 | 6214 | 9990-9969 | 51.3 | 51.3 | 371 |
| 6120 | 10009-10027 | 6215 | 10188-10171 | 50.2 | 50.2 | 180 |
| 6121 | 10093-10113 | 6216 | 10244-10223 | 52.4 | 50.6 | 152 |
| 6122 | 10242-10265 | 6217 | 10608-10589 | 51.2 | 51.0 | 367 |
| 6123 | 10549-10571 | 6218 | 10783-10763 | 53.7 | 55.2 | 235 |
| 6124 | 10766-10785 | 6219 | 10930-10912 | 52.0 | 51.1 | 165 |
| 6125 | 11065-11085 | 6220 | 11305-11287 | 50.7 | 50.0 | 241 |
| 6126 | 11265-11287 | 6221 | 11429-11405 | 54.5 | 53.5 | 165 |
| 6127 | 11552-11571 | 6222 | 11730-11709 | 52.0 | 50.4 | 179 |
| 6128 | 11705-11726 | 6223 | 11869-11848 | 50.1 | 50.2 | 165 |
| 6129 | 11801-11824 | 6224 | 11984-11967 | 51.5 | 50.4 | 184 |
| 6130 | 12040-12058 | 6225 | 12254-12235 | 52.3 | 51.9 | 215 |
| 6131 | 12235-12253 | 6226 | 12406-12388 | 50.1 | 50.1 | 172 |
| 6132 | 12366-12384 | 6227 | 12730-12712 | 51.7 | 52.2 | 365 |
| 6133 | 12727-12748 | 6228 | 12994-12976 | 50.8 | 50.3 | 268 |
| 6134 | 12948-12966 | 6229 | 13224-13201 | 50.7 | 51.7 | 277 |
| 6135 | 13175-13196 | 6230 | 13324-13300 | 54.3 | 55.1 | 150 |
| 6136 | 13237-13258 | 6231 | 13545-13526 | 52.9 | 52.9 | 309 |
| 6137 | 13790-13810 | 6232 | 13963-13945 | 50.9 | 50.7 | 174 |
| 6138 | 14080-14098 | 6233 | 14280-14257 | 51.5 | 51.0 | 201 |
| 6139 | 14405-14427 | 6234 | 14561-14540 | 50.2 | 50.9 | 157 |
| 6140 | 14882-14906 | 6235 | 15046-15024 | 50.9 | 51.5 | 165 |
| 6141 | 14951-14976 | 6236 | 15145-15124 | 53.1 | 52.9 | 195 |
| 6142 | 15113-15134 | 6237 | 15275-15257 | 51.6 | 50.8 | 163 |
| 6143 | 15211-15230 | 6238 | 15383-15363 | 50.2 | 50.1 | 173 |
| 6144 | 15364-15387 | 6239 | 15528-15506 | 54.0 | 52.1 | 165 |
| 6145 | 15456-15477 | 6240 | 15605-15585 | 52.0 | 53.2 | 150 |
| 6146 | 15513-15532 | 6241 | 15897-15876 | 51.2 | 50.4 | 385 |
| 6147 | 15837-15856 | 6242 | 15999-15978 | 52.3 | 50.8 | 163 |
| 6148 | 16073-16096 | 6243 | 16301-16277 | 51.7 | 52.8 | 229 |
| 6149 | 16245-16266 | 6244 | 16404-16380 | 50.3 | 52.0 | 160 |
| 6150 | 16366-16385 | 6245 | 16515-16492 | 52.9 | 53.8 | 150 |
| 6151 | 16553-16571 | 6246 | 16777-16758 | 53.4 | 51.5 | 225 |
| 6152 | 16832-16852 | 6247 | 17026-17004 | 51.0 | 51.6 | 195 |
| 6153 | 16982-17001 | 6248 | 17359-17340 | 51.2 | 50.2 | 378 |
| 6154 | 17354-17372 | 6249 | 17511-17490 | 51.3 | 50.4 | 158 |
| 6155 | 17422-17443 | 6250 | 17573-17552 | 50.2 | 51.1 | 152 |
| 6156 | 17603-17623 | 6251 | 17769-17748 | 50.7 | 51.5 | 167 |
| 6157 | 17728-17746 | 6252 | 17883-17862 | 50.9 | 51.2 | 156 |
| 6158 | 18011-18030 | 6253 | 18163-18140 | 52.9 | 51.9 | 153 |
| 6159 | 18076-18098 | 6254 | 18225-18205 | 54.4 | 55.0 | 150 |
| 6160 | 18270-18292 | 6255 | 18432-18413 | 51.9 | 51.4 | 163 |
| 6161 | 18352-18373 | 6256 | 18648-18629 | 51.3 | 50.8 | 297 |
| 6162 | 18550-18571 | 6257 | 18702-18684 | 50.4 | 51.9 | 153 |
| 6163 | 18720-18738 | 6258 | 19004-18983 | 50.6 | 51.0 | 285 |
| 6164 | 18960-18981 | 6259 | 19109-19085 | 54.7 | 54.3 | 150 |
| 6165 | 19065-19089 | 6260 | 19217-19195 | 52.8 | 51.7 | 153 |
| 6166 | 19310-19329 | 6261 | 19476-19454 | 50.2 | 52.1 | 167 |
| 6167 | 19569-19589 | 6262 | 19719-19701 | 50.5 | 51.8 | 151 |
| 6168 | 19707-19731 | 6263 | 19856-19833 | 55.7 | 55.9 | 150 |
| 6169 | 19771-19792 | 6264 | 19921-19901 | 50.1 | 50.2 | 151 |
| 6170 | 19833-19851 | 6265 | 19986-19966 | 50.9 | 50.7 | 154 |

TABLE 6
Primers

| | | | | T_{M} | | Product |
|---|---|---|---|---|---|---|
| Forward primer SEQ | | Reverse primer SEQ | | (FOR & REV) | | length |
| ID NO & Co-ordinates | | ID NO & Co-ordinates | | (° C.) | | (bp) |
| 6266 | 20110-20132 | 6305 | 20425-20404 | 51.9 | 50.9 | 316 |
| 6267 | 20468-20492 | 6306 | 20617-20596 | 53.2 | 53.5 | 150 |
| 6268 | 20557-20578 | 6307 | 20891-20871 | 50.4 | 50.6 | 335 |
| 6269 | 20838-20856 | 6308 | 21037-21015 | 52.5 | 52.0 | 200 |
| 6270 | 21096-21116 | 6309 | 21295-21272 | 50.1 | 51.7 | 200 |
| 6271 | 22173-22194 | 6310 | 22414-22395 | 52.4 | 51.0 | 242 |
| 6272 | 22320-22342 | 6311 | 22501-22479 | 54.8 | 54.3 | 182 |
| 6273 | 22532-22552 | 6312 | 22695-22675 | 50.6 | 50.0 | 164 |
| 6274 | 22712-22736 | 6313 | 22873-22852 | 56.7 | 55.5 | 162 |
| 6275 | 22842-22861 | 6314 | 23086-23067 | 51.0 | 52.8 | 245 |
| 6276 | 23151-23170 | 6315 | 23395-23376 | 51.4 | 50.3 | 245 |
| 6277 | 23307-23326 | 6316 | 23524-23501 | 51.1 | 51.1 | 218 |
| 6278 | 23615-23635 | 6317 | 23776-23758 | 50.7 | 50.2 | 162 |
| 6279 | 23838-23857 | 6318 | 23996-23977 | 50.4 | 50.6 | 159 |
| 6280 | 24030-24051 | 6319 | 24407-24386 | 57.6 | 55.7 | 378 |
| 6281 | 24388-24407 | 6320 | 24581-24563 | 50.4 | 50.1 | 194 |
| 6282 | 24559-24579 | 6321 | 24938-24921 | 52.0 | 50.4 | 380 |
| 6283 | 24922-24941 | 6322 | 25184-25166 | 50.1 | 51.2 | 263 |
| 6284 | 25201-25220 | 6323 | 25400-25382 | 51.1 | 51.4 | 200 |
| 6285 | 25363-25381 | 6324 | 25646-25627 | 51.1 | 50.5 | 284 |
| 6286 | 25656-25681 | 6325 | 25839-25814 | 54.5 | 56.4 | 184 |
| 6287 | 25761-25782 | 6326 | 25982-25961 | 54.6 | 54.3 | 222 |
| 6288 | 26039-26058 | 6327 | 26189-26166 | 54.0 | 53.0 | 151 |
| 6289 | 26184-26205 | 6328 | 26333-26310 | 50.9 | 51.8 | 150 |
| 6290 | 26422-26442 | 6329 | 26660-26641 | 51.3 | 50.2 | 239 |
| 6291 | 26571-26589 | 6330 | 26739-26715 | 51.7 | 53.2 | 169 |
| 6292 | 26733-26752 | 6331 | 26960-26941 | 51.1 | 52.2 | 228 |
| 6293 | 26866-26885 | 6332 | 27139-27117 | 50.7 | 51.9 | 274 |
| 6294 | 27300-27321 | 6333 | 27458-27439 | 51.2 | 50.2 | 159 |
| 6295 | 27361-27380 | 6334 | 27579-27558 | 52.4 | 51.1 | 219 |
| 6296 | 27718-27740 | 6335 | 27917-27901 | 50.7 | 50.0 | 200 |
| 6297 | 28041-28059 | 6336 | 28207-28189 | 50.8 | 50.8 | 167 |
| 6298 | 28166-28189 | 6337 | 28411-28393 | 52.2 | 52.9 | 246 |
| 6299 | 28395-28414 | 6338 | 28671-28653 | 51.5 | 50.2 | 277 |
| 6300 | 28654-28672 | 6339 | 28821-28800 | 50.6 | 52.3 | 168 |
| 6301 | 28867-28885 | 6340 | 29184-29166 | 51.5 | 51.6 | 318 |
| 6302 | 29183-29204 | 6341 | 29360-29342 | 50.4 | 50.4 | 178 |
| 6303 | 29262-29279 | 6342 | 29626-29606 | 50.1 | 50.2 | 365 |
| 6304 | 29538-29559 | 6343 | 29690-29670 | 50.0 | 50.4 | 153 |

TABLE 7
Primers

| Name | SEQ ID NO: | Co-ordinates |
|---|---|---|
| AB4f | 6344 | 19869-19888 |
| AB5f | 6345 | 20238-20257 |
| BC1f | 6346 | 20581-20600 |
| BC2f | 6347 | 20950-20969 |
| BC3f | 6348 | 21339-21358 |
| BC4f | 6349 | 21708-21727 |
| BC5f | 6350 | 22041-22060 |
| BC6f | 6351 | 22410-22429 |
| BC7f | 6352 | 22759-22778 |
| BC8f | 6353 | 23131-23150 |
| BC9f | 6354 | 23500-23519 |
| BC10f | 6355 | 23841-23860 |
| BC11f | 6356 | 24210-24229 |
| BC12f | 6357 | 24560-24579 |
| BC13f | 6358 | 24941-24960 |
| BC14f | 6359 | 25310-25329 |
| BC15f | 6360 | 25675-25694 |
| BC16f | 6361 | 26044-26063 |
| BC17f | 6362 | 26413-26432 |
| BC18f | 6363 | 26763-26782 |
| BC19f | 6364 | 27132-27151 |
| BC20f | 6365 | 27491-27510 |
| BC21f | 6366 | 27845-27864 |
| CB1r | 6367 | 28011-28030 |
| CB2r | 6368 | 27671-27690 |
| CB3r | 6369 | 27301-27320 |
| CB4r | 6370 | 26931-26950 |
| CB5r | 6371 | 26575-26594 |
| CB6r | 6372 | 26191-26210 |
| CB7r | 6373 | 25841-25860 |
| CB8r | 6374 | 25476-25495 |
| CB9r | 6375 | 25126-25145 |
| CB10r | 6376 | 24791-24810 |
| CB11r | 6377 | 24422-24441 |
| CB12r | 6378 | 24031-24050 |
| CB13r | 6379 | 23673-23692 |
| CB14r | 6380 | 23298-23317 |
| CB15r | 6381 | 22928-22947 |
| CB16r | 6382 | 22567-22586 |
| CB17r | 6383 | 22196-22215 |
| CB18r | 6384 | 21831-21850 |
| CB19r | 6385 | 21431-21450 |
| CB20r | 6386 | 21073-21092 |
| CB21r | 6387 | 20715-20734 |
| BA1r | 6388 | 20345-20364 |
| BA2r | 6389 | 19969-19988 |
| BA3r | 6390 | 19599-19618 |
| BA4r | 6391 | 19228-19247 |
| BA5r | 6392 | 18852-18871 |

TABLE 8
Primers

| | | |
|---|---|---|
| Name | SEQ ID NO | Co-ordinates |

| | | |
|---|---|---|
| F1 | 6393 | 1-19 |
| F2 | 6394 | 292-310 |
| F3 | 6395 | 721-742 |
| F4 | 6396 | 984-1003 |
| F5 | 6397 | 1420-1441 |
| F6 | 6398 | 1740-1764 |
| F7 | 6399 | 2226-2245 |
| F8 | 6400 | 2742-2763 |
| F9 | 6401 | 3007-3031 |
| F10 | 6402 | 3453-3476 |
| F11 | 6403 | 4007-4027 |
| F12 | 6404 | 4366-4387 |
| F13 | 6405 | 4658-4677 |
| F14 | 6406 | 5239-5260 |
| F15 | 6407 | 5593-5612 |
| F16 | 6408 | 6271-6291 |
| F17 | 6409 | 7253-7272 |
| F18 | 6410 | 7615-7635 |
| F19 | 6411 | 7845-7867 |
| F20 | 6412 | 8143-8166 |
| F21 | 6413 | 8553-8575 |
| F22 | 6414 | 9244-9267 |
| F23 | 6415 | 10009-10027 |
| F24 | 6416 | 10242-10265 |
| F25 | 6417 | 10766-10785 |
| F26 | 6418 | 11265-11287 |
| F27 | 6419 | 11705-11726 |
| F28 | 6420 | 12040-12058 |
| F29 | 6421 | 12366-12384 |
| F30 | 6422 | 12948-12966 |
| F31 | 6423 | 13237-13258 |
| F32 | 6424 | 14080-14098 |
| F33 | 6425 | 14882-14906 |
| F34 | 6426 | 15113-15134 |
| F35 | 6427 | 15364-15387 |
| F36 | 6428 | 15513-15532 |
| F37 | 6429 | 16073-16096 |
| F38 | 6430 | 16366-16385 |
| F39 | 6431 | 16832-16852 |
| F40 | 6432 | 17354-17372 |
| F41 | 6433 | 17603-17623 |
| F42 | 6434 | 18011-18030 |
| F43 | 6435 | 18270-18292 |
| F44 | 6436 | 18550-18571 |
| F45 | 6437 | 18960-18981 |
| F46 | 6438 | 19310-19329 |
| F47 | 6439 | 19707-19731 |
| F48 | 6440 | 19833-19851 |
| R1 | 6441 | 334-315 |
| R2 | 6442 | 749-731 |
| R3 | 6443 | 1077-1058 |
| R4 | 6444 | 1479-1460 |
| R5 | 6445 | 1834-1811 |
| R6 | 6446 | 2251-2232 |
| R7 | 6447 | 2749-2728 |
| R8 | 6448 | 3082-3058 |
| R9 | 6449 | 3497-3478 |
| R10 | 6450 | 3877-3853 |
| R11 | 6451 | 4316-4295 |
| R12 | 6452 | 4708-4690 |
| R13 | 6453 | 5115-5092 |
| R14 | 6454 | 5560-5542 |
| R15 | 6455 | 6291-6271 |
| R16 | 6456 | 7171-7153 |
| R17 | 6457 | 7677-7654 |
| R18 | 6458 | 7936-7915 |
| R19 | 6459 | 8189-8170 |
| R20 | 6460 | 8388-8369 |
| R21 | 6461 | 9254-9236 |
| R22 | 6462 | 9990-9969 |
| R23 | 6463 | 10244-10223 |
| R24 | 6464 | 10783-10763 |
| R25 | 6465 | 11305-11287 |
| R26 | 6466 | 11730-11709 |
| R27 | 6467 | 11984-11967 |
| R28 | 6468 | 12406-12388 |
| R29 | 6469 | 12994-12976 |
| R30 | 6470 | 13324-13300 |
| R31 | 6471 | 13963-13945 |
| R32 | 6472 | 14561-14540 |
| R33 | 6473 | 15145-15124 |
| R34 | 6474 | 15383-15363 |
| R35 | 6475 | 15605-15585 |
| R36 | 6476 | 15999-15978 |
| R37 | 6477 | 16404-16380 |
| R38 | 6478 | 16777-16758 |
| R39 | 6479 | 17359-17340 |
| R40 | 6480 | 17573-17552 |
| R41 | 6481 | 17883-17862 |
| R42 | 6482 | 18225-18205 |
| R43 | 6483 | 18648-18629 |
| R44 | 6484 | 19004-18983 |
| R45 | 6485 | 19217-19195 |
| R46 | 6486 | 19719-19701 |
| R47 | 6487 | 19921-19901 |

TABLE 9
Primers

| | Name | SEQ ID NO: |
|---|---|---|
| 1 | CB12R | 6488 |
| 2 | R0010 | 6489 |
| 3 | R0011 | 6490 |
| 4 | R0012 | 6491 |
| 5 | BNI-ED | 6492 |
| 6 | BNI-EU | 6493 |
| 7 | SAR1S-U | 6494 |
| 8 | SAR1As-D | 6495 |
| 9 | SAR1S | 6496 |
| 10 | SAR1As | 6497 |
| 11 | IN2-U | 6498 |
| 12 | IN4-D | 6499 |
| 13 | IN-2 | 6500 |
| 14 | IN-4 | 6501 |
| 15 | IN-6 | 6502 |
| 16 | IN-7 | 6503 |
| 17 | COR1-U | 6504 |
| 18 | COR2-D | 6505 |
| 19 | COR-1 | 6506 |
| 20 | COR-2 | 6507 |
| 21 | HKUF-U | 6508 |
| 22 | HKUR-D | 6509 |
| 23 | HKU-F | 6510 |
| 24 | HKU-R | 6511 |
| 25 | 1451-D | 6512 |
| 26 | 1451-U | 6513 |
| 27 | 690-D | 6514 |
| 28 | 690-U | 6515 |
| 29 | 690-D2 | 6516 |
| 30 | 690-U2 | 6517 |
| 31 | EMC7-D | 6518 |
| 32 | EMC7-U | 6519 |
| 33 | EMC7-D2 | 6520 |
| 34 | EMC7-U2 | 6521 |
| 35 | EMC8-D | 6522 |
| 36 | EMC8-U | 6523 |
| 37 | EMC8-D2 | 6524 |
| 38 | EMC8-U2 | 6525 |
| 39 | EMC11-D | 6526 |
| 40 | EMC11-U | 6527 |
| 41 | ORF1B-D | 6528 |
| 42 | ORF1B-U | 6529 |
| 43 | ORFS-D | 6530 |
| 44 | ORES-U | 6531 |
| 45 | E7-717F | 6532 |
| 46 | E8-85R | 6533 |
| 47 | E8-307F | 6534 |
| 48 | E11-771F | 6535 |
| 49 | E11-96R | 6536 |
| 50 | CON1-F | 6537 |
| 51 | CON1-U | 6538 |
| 52 | CON2-F | 6539 |
| 53 | CON2-R | 6540 |
| 54 | CON3-F | 6541 |
| 55 | CON3-R | 6542 |
| 56 | 15-F | 6543 |
| 57 | 15-R | 6544 |
| 58 | 15-F2 | 6545 |
| 59 | 15-R2 | 6546 |
| 60 | 13-F | 6547 |
| 61 | 13-R | 6548 |
| 62 | 13-F2 | 6549 |
| 63 | 13-R2 | 6550 |
| 64 | CONTIG-F | 6551 |
| 65 | QT3-R | 6552 |
| 66 | QT3-F | 6553 |
| 67 | QIN-R | 6554 |
| 68 | QIN-F | 6555 |
| 69 | AB1-F | 6556 |
| 70 | AB2-F | 6557 |
| 71 | AB3-F | 6558 |
| 72 | AB1-R | 6559 |

TABLE 10
Features of the predicted proteins and open reading frames of the SARS virus

| | SARS ORF | Length | | Cleavage | | |
|---|---|---|---|---|---|---|
| | (SEQ ID NO) | (aa) | Role | site | Features | Cons^{d*} |
| ORF1a | P28 (9766) | 179 | Leader protein | 179 (G/G)^{#} | | + |
| | P65 (9767) | 639 | Homologue of MHV p65 | 818 (G/A) | | + |
| | | | cleavage product | | | |
| | Nsp1 | 2422^{##} | Papain like protease, | 3240 (Q/S) | phosphoesterase domain | + |
| | (9768) | | cleaves the first two | | Zn binding domain | |
| | | | proteins | | | |
| | Nsp2 | 306 | 3C-like protease, cleaves | 3546 (Q/G) | | + |
| | (9769) | | proteins nsp1-nsp12 | | | |
| | Nsp3 (9770) | 290 | ? | 3836 (Q/S) | 5 TMDs | + |
| | Nsp4 (9771) | 83 | ? | 3919 (Q/A) | 1 TMD | + |
| | Nsp5 (9772) | 198 | ? | 4117 (Q/N) | | + |
| | Nsp6 (9773) | 113 | ? | 4230 (Q/A) | | + |
| | Nsp7 (9774) | 139 | ? | 4369 (Q/S) | Putative growth factor-like motif | + |
| ORF1b | Nsp9 (9775) | 932 | RNA polymerase | 5298 (Q/A) | | + |
| | Nsp10 (9776) | 601 | Putative helicase | 5899 (Q/A) | Metal binding domain, | + |
| | | | Tanner et al. (2003) J Biol Chem | | ATP/GTP binding domain | |
| | | | 278: 39578-82 | | | |
| | Nsp11 (9777) | 527 | ? | 6426 (Q/S) | | + |
| | Nsp12 (9778) | 346 | ? | 6772 (Q/A) | | + |
| | Nsp13 (9779) | 298 | ? | — | | + |
| Structural | Spike (S) | | Major antigenic | | Leader peptide, 1 TMD, 17 N- | + |
| region | (6042) | | determinant, contains the | | glycosylation sites | |
| | | | receptor-binding domain | | | |
| | Orf3 (6043) | 274 | ? | | 2 TMDs, 1 N-glycosylation site, 10 | − |
| | | | | | O-glycosylation sites | |
| | Orf4 (6044) | 154 | ? | | | − |
| | Envelope (E) | 76 | Associated with viral | | 1 TMD, 2 N-glycosylation sites | + |
| | (6045) | | envelope | | | |
| | Matrix (M) | 221 | Associated with viral | | 3 TMDs, 1 N-glycosylation site | + |
| | (6046) | | envelope, membrane | | | |
| | | | spanning protein | | | |
| | Orf7 | 63 | ? | | 1 TMD | − |
| | (6047) | | | | | |
| | Orf8 (6048) | 122 | ? | | 1 TMD | − |
| | Orf9 (6049) | 44 | ? | | Surface-associated | − |
| | Orf10 | 39 | ? | | Surface-associated | − |
| | Orf11(6050) | 84 | ? | | 1 N-glycosylation site | − |
| | Nucleocapsid (N) | 422 | Associated with viral | | phosphoprotein | + |
| | (6052) | | genomic RNA | | | |
| | Orf13 | 98 | ? | | 1 O-glycosylation site | − |

TMD: predicted transmembrane domain.
Cons^{d}*: + indicates presence of corresponding protein at least in one of the other coronaviruses
^{#}Alternatively, cleaved after Gly-Gly (i.e. at G/A) to give a 180mer
^{##}This 2422mer may be further cleaved after residue 1922 (Gly-2740 of SEQ ID NO: 6039) to give a 1922mer PLpro containing the Zn-binding motif (SEQ ID NO: 7254) and a 500mer.

TABLE 11
Protein homologies between SARS and other coronaviruses

| | group 1 | | | group 2 | | group 3 |
|---|---|---|---|---|---|---|
| Proteins | 229E | TGV | PEDV | MHV | BCoV | AIBV |

REPLICASE REGION

| | | | | | | |
|---|---|---|---|---|---|---|
| leader protein | <20 | <20 | <20 | 27 | <20 | <20 |
| p28 | | | | | | |
| p65 homologue | <20 | 23 | 23 | <20 | 20 | <20 |
| nsp1 | 25.5 | 25.8 | 25.4 | 29 | 30 | 25 |
| (PLP protease) | | | | | | |
| nsp2 | 40.4 | 43.8 | 44.6 | 50 | 48.4 | 41 |
| (3CL protease) | | | | | | |
| nsp3 | 30 | 27 | 29.4 | 34.2 | 35.5 | 28.5 |
| nsp4 | 38.6 | 42.2 | 39.8 | 47.5 | 46.1 | 37.3 |
| nsp5 | 48.2 | 42.9 | 43.9 | 46.8 | 47.3 | 38.7 |
| nsp6 | 45.1 | 38.9 | 45.1 | 45.1 | 46.9 | 39.8 |
| nsp7 | 53.8 | 54.5 | 56.1 | 56.2 | 55.4 | 58.3 |
| nsp9 | 59.8 | 59.6 | 60 | 67.3 | 66.9 | 62.4 |
| (polymerase) | | | | | | |
| nsp10 | 60.7 | 62 | 62.3 | 67.2 | 68.6 | 58.9 |
| (helicase) | | | | | | |
| nsp11 | 52.3 | 53.7 | 52.3 | 57.6 | 57.6 | 52 |
| nsp12 | 43.1 | 43 | 45.4 | 45.9 | 45 | 40.2 |
| nsp13 | 56.4 | 54.4 | 55.3 | 63 | 65 | 53.4 |

STRUCTURAL REGION

| | | | | | | |
|---|---|---|---|---|---|---|
| Spike (S) | 28.8 | 31.6* | 30.3 | 31.1 | 31 | 32.7* |
| Envelope (E) | 33* | 27.9 | 20 | 23 | 26.5 | 23.2 |
| Matrix | 30.6 | 32.5 | 34.8 | 40.8 | 41.9 | 32.5 |
| glycoprotein | | | | | | |
| (M) | | | | | | |
| Nucleocapsid | 26.9 | 30.1 | 29.5 | 37.3 | 37.4 | 31.5 |
| (N) | | | | | | |

*These three alignments were obtained only on a fragment of the whole protein.
Numbers indicate percentage of aminoacid identity between SARS proteins and corresponding gene products of other coronaviruses. More conserved pairs are in bold; more variable pairs are underlined.

TABLE 12
Nucleotide and aminoacid differences between five SARS isolates

| | | FRA* | TOR2* | Urbani* | CUHK* | HKU* |
|---|---|---|---|---|---|---|
| | position° | base/aminoacid | base/aminoacid | base/aminoacid | base/aminoacid | base/aminoacid |
| ORF1a | 2557 | A/Thr | G/Ala | G/Ala | G/Ala | G/Ala |
| | 2601 | T/Val | | | | C |
| | 7746 | G/Pro | | | T | |
| | 7919 | C/Ala | | T/Val | | |
| | 7930 | G/Asp | | | | A/Asn |
| | 8387 | G/Ser | | | | C/Thr |
| | 8416 | G/Arg | | | | C/Thr |
| | 9404 | T/Val | | | C/Ala | |
| | 9479 | T/Val | | | C/Ala | |
| | 11448 | T/Ile | C | C | C | C |
| ORF1b | 13494 | GT/Val | | | | AG/Ser |
| | 16622 | C/Ala | | T | | |
| | 17564 | T/Asp | | | C/Glu | |
| | 17846 | C/Arg | | | T | |
| | 18065 | G/Lys | | | | A |
| | 18965 | A/Ile | T | T | T | T |
| | 19064 | A/Glu | | G | G | |
| | 19084 | T/Ile | C/Thr | C/Thr | C/Thr | C/Thr |
| spike | 21721 | G/Gly | | | A/Asp | |
| | 22222 | T/Ile | | | C/Thr | |
| | 23220 | T/Ser | G/Ala | | | |
| | 24872 | T/Leu | | C | | |
| | 24933 | T/Phe | C/Leu | C/Leu | C/Leu | C/Leu |
| ORF3 | 25298 | G/Gly | A/Arg | | | |
| | 25569 | T/Met | | | | A/Lys |
| matrix | 26600 | T/Val | C/Ala | C/Ala | C/Ala | |
| | 26857 | T/Ser | | C/Pro | | |
| ORF10 | 27827 | T/Cys | | | C/Arg | |
| nucleocapsid | 28268 | T/Ile | C/Thr | C/Thr | C/Thr | C/Thr |

*SARS coronavirus FRA (accession number AY310120)
SARS coronavirus TOR2 (accession number AY274119)
SARS coronavirus Urbani (accession number AY278741)
SARS coronavirus CUHK-W1 (accession number AY278554)
SARS coronavirus HKU-39849 (accession number AY278491)
°The position is based on the FRA sequence.

TABLES 13-25: T-epitope predictions for SEQ ID NOS: 6039-6050 & 6052

Epitope predictions were performed at http://www.mpiib-berlin.mpg.de/MAPPP/binding.html using a minimum score of 0.5 and the BIMAS matrix, with a maximum of 20 results being selected. The analysis revealed 9 mer and 10 mer epitopes.

TABLE 13
Epitopes for SEQ ID NO: 6039

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 1867 | SEQ ID NO: 7400 | 8% | 450 |
| 2 | 4139 | SEQ ID NO: 7401 | 5.55% | 312.5 |
| 3 | 88 | SEQ ID NO: 7402 | 4% | 225 |
| 4 | 4249 | SEQ ID NO: 7403 | 3.55% | 200 |
| 5 | 4059 | SEQ ID NO: 7404 | 2.22% | 125 |
| 6 | 2027 | SEQ ID NO: 7405 | 1.6% | 90 |
| 7 | 3413 | SEQ ID NO: 7406 | 1.11% | 62.5 |
| 8 | 1823 | SEQ ID NO: 7407 | 0.88% | 50 |
| 9 | 2798 | SEQ ID NO: 7408 | 0.88% | 50 |
| 10 | 220 | SEQ ID NO: 7409 | 0.8% | 45 |
| 11 | 3738 | SEQ ID NO: 7410 | 0.8% | 45 |
| 12 | 4182 | SEQ ID NO: 7411 | 0.8% | 45 |
| 13 | 4174 | SEQ ID NO: 7412 | 0.66% | 37.5 |
| 14 | 1940 | SEQ ID NO: 7413 | 0.55% | 31.25 |
| 15 | 38 | SEQ ID NO: 7414 | 0.48% | 27 |
| 16 | 1231 | SEQ ID NO: 7415 | 0.44% | 25 |
| 17 | 1613 | SEQ ID NO: 7416 | 0.44% | 25 |
| 18 | 3645 | SEQ ID NO: 7417 | 0.44% | 25 |
| 19 | 4192 | SEQ ID NO: 7418 | 0.44% | 25 |
| 20 | 378 | SEQ ID NO: 7419 | 0.4% | 22.5 |

HLA A1 - 10 mers

| Maximum possible score using this molecule type | | | | 5625 |
|---|---|---|---|---|
| 1 | 1867 | SEQ ID NO: 7420 | 8% | 450 |
| 2 | 1495 | SEQ ID NO: 7421 | 4% | 225 |
| 3 | 3921 | SEQ ID NO: 7422 | 2.4% | 135 |
| 4 | 486 | SEQ ID NO: 7423 | 2.22% | 125 |
| 5 | 4139 | SEQ ID NO: 7424 | 2.22% | 125 |
| 6 | 62 | SEQ ID NO: 7425 | 1.6% | 90 |
| 7 | 1190 | SEQ ID NO: 7426 | 1.6% | 90 |
| 8 | 1284 | SEQ ID NO: 7427 | 1.6% | 90 |
| 9 | 3284 | SEQ ID NO: 7428 | 1.6% | 90 |
| 10 | 2921 | SEQ ID NO: 7429 | 1.2% | 67.5 |
| 11 | 349 | SEQ ID NO: 7430 | 0.8% | 45 |
| 12 | 789 | SEQ ID NO: 7431 | 0.8% | 45 |
| 13 | 1185 | SEQ ID NO: 7432 | 0.8% | 45 |
| 14 | 4184 | SEQ ID NO: 7433 | 0.8% | 45 |
| 15 | 1313 | SEQ ID NO: 7434 | 0.64% | 36 |
| 16 | 3948 | SEQ ID NO: 7435 | 0.48% | 27 |
| 17 | 149 | SEQ ID NO: 7436 | 0.44% | 25 |
| 18 | 941 | SEQ ID NO: 7437 | 0.44% | 25 |
| 19 | 1390 | SEQ ID NO: 7438 | 0.44% | 25 |
| 20 | 1613 | SEQ ID NO: 7439 | 0.44% | 25 |

HLA A3 - 9 mers

| Maximum possible score using this molecule type | | | | 12150 |
|---|---|---|---|---|
| 1 | 1010 | SEQ ID NO: 7440 | 1.48% | 180 |
| 2 | 3155 | SEQ ID NO: 7441 | 1.48% | 180 |
| 3 | 1229 | SEQ ID NO: 7442 | 1.23% | 150 |
| 4 | 2405 | SEQ ID NO: 7443 | 0.88% | 108 |
| 5 | 2 | SEQ ID NO: 7444 | 0.74% | 90 |
| 6 | 2304 | SEQ ID NO: 7445 | 0.74% | 90 |
| 7 | 2358 | SEQ ID NO: 7446 | 0.74% | 90 |
| 8 | 3160 | SEQ ID NO: 7447 | 0.74% | 90 |
| 9 | 3771 | SEQ ID NO: 7448 | 0.74% | 90 |
| 10 | 4007 | SEQ ID NO: 7449 | 0.74% | 90 |
| 11 | 3079 | SEQ ID NO: 7450 | 0.66% | 81 |
| 12 | 4045 | SEQ ID NO: 7451 | 0.66% | 81 |
| 13 | 1081 | SEQ ID NO: 7452 | 0.49% | 60 |
| 14 | 3268 | SEQ ID NO: 7453 | 0.49% | 60 |
| 15 | 4144 | SEQ ID NO: 7454 | 0.49% | 60 |
| 16 | 614 | SEQ ID NO: 7455 | 0.37% | 45 |
| 17 | 728 | SEQ ID NO: 7456 | 0.37% | 45 |
| 18 | 1537 | SEQ ID NO: 7457 | 0.37% | 45 |
| 19 | 313 | SEQ ID NO: 7458 | 0.32% | 40 |
| 20 | 1744 | SEQ ID NO: 7459 | 0.32% | 40 |

HLA A3 - 10 mers

| Maximum possible score using this molecule type | | | | 12150 |
|---|---|---|---|---|
| 1 | 62 | SEQ ID NO: 7460 | 4.44% | 540 |
| 2 | 2151 | SEQ ID NO: 7461 | 2.46% | 300 |
| 3 | 633 | SEQ ID NO: 7462 | 2.22% | 270 |
| 4 | 1158 | SEQ ID NO: 7463 | 2.22% | 270 |
| 5 | 2565 | SEQ ID NO: 7464 | 2.22% | 270 |
| 6 | 2298 | SEQ ID NO: 7465 | 1.77% | 216 |
| 7 | 3159 | SEQ ID NO: 7466 | 1.11% | 135 |
| 8 | 640 | SEQ ID NO: 7467 | 0.98% | 120 |
| 9 | 2186 | SEQ ID NO: 7468 | 0.74% | 90 |
| 10 | 3869 | SEQ ID NO: 7469 | 0.74% | 90 |
| 11 | 2308 | SEQ ID NO: 7470 | 0.66% | 81 |
| 12 | 786 | SEQ ID NO: 7471 | 0.55% | 67.5 |
| 13 | 749 | SEQ ID NO: 7472 | 0.49% | 60 |
| 14 | 1080 | SEQ ID NO: 7473 | 0.49% | 60 |
| 15 | 2358 | SEQ ID NO: 7474 | 0.49% | 60 |
| 16 | 3955 | SEQ ID NO: 7475 | 0.49% | 60 |
| 17 | 714 | SEQ ID NO: 7476 | 0.37% | 45 |
| 18 | 1081 | SEQ ID NO: 7477 | 0.37% | 45 |
| 19 | 1170 | SEQ ID NO: 7478 | 0.37% | 45 |
| 20 | 1228 | SEQ ID NO: 7479 | 0.37% | 45 |

HLA A24 - 9 mers

| Maximum possible score using this molecule type | | | | 1596.672 |
|---|---|---|---|---|
| 1 | 3797 | SEQ ID NO: 7480 | 37.57% | 600 |
| 2 | 4202 | SEQ ID NO: 7481 | 37.57% | 600 |
| 3 | 3189 | SEQ ID NO: 7482 | 25.05% | 400 |
| 4 | 1864 | SEQ ID NO: 7483 | 23.14% | 369.6 |
| 5 | 1066 | SEQ ID NO: 7484 | 22.54% | 360 |
| 6 | 2143 | SEQ ID NO: 7485 | 22.54% | 360 |
| 7 | 2693 | SEQ ID NO: 7486 | 22.54% | 360 |
| 8 | 1426 | SEQ ID NO: 7487 | 18.78% | 300 |
| 9 | 1238 | SEQ ID NO: 7488 | 18.03% | 288 |
| 10 | 3768 | SEQ ID NO: 7489 | 18.03% | 288 |
| 11 | 797 | SEQ ID NO: 7490 | 15.03% | 240 |
| 12 | 1882 | SEQ ID NO: 7491 | 15.03% | 240 |
| 13 | 1490 | SEQ ID NO: 7492 | 13.77% | 220 |
| 14 | 2237 | SEQ ID NO: 7493 | 13.77% | 220 |
| 15 | 95 | SEQ ID NO: 7494 | 12.52% | 200 |
| 16 | 1821 | SEQ ID NO: 7495 | 12.52% | 200 |
| 17 | 2289 | SEQ ID NO: 7496 | 12.52% | 200 |
| 18 | 3080 | SEQ ID NO: 7497 | 12.52% | 200 |
| 19 | 3660 | SEQ ID NO: 7498 | 12.52% | 200 |
| 20 | 4354 | SEQ ID NO: 7499 | 12.52% | 200 |

HLA A24 - 10 mers

| Maximum possible score using this molecule type | | | | 1596.672 |
|---|---|---|---|---|
| 1 | 2143 | SEQ ID NO: 7500 | 37.87% | 604.8 |
| 2 | 1159 | SEQ ID NO: 7501 | 26.30% | 420 |
| 3 | 1650 | SEQ ID NO: 7502 | 26.30% | 420 |
| 4 | 1150 | SEQ ID NO: 7503 | 18.78% | 300 |
| 5 | 2763 | SEQ ID NO: 7504 | 18.78% | 300 |
| 6 | 3165 | SEQ ID NO: 7505 | 18.78% | 300 |
| 7 | 3201 | SEQ ID NO: 7506 | 15.03% | 240 |
| 8 | 3694 | SEQ ID NO: 7507 | 15.03% | 240 |
| 9 | 4204 | SEQ ID NO: 7508 | 15.03% | 240 |
| 10 | 1692 | SEQ ID NO: 7509 | 13.77% | 220 |
| 11 | 797 | SEQ ID NO: 7510 | 12.52% | 200 |
| 12 | 1610 | SEQ ID NO: 7511 | 12.52% | 200 |
| 13 | 1789 | SEQ ID NO: 7512 | 12.52% | 200 |
| 14 | 1881 | SEQ ID NO: 7513 | 12.52% | 200 |
| 15 | 3090 | SEQ ID NO: 7514 | 12.52% | 200 |
| 16 | 3763 | SEQ ID NO: 7515 | 12.52% | 200 |
| 17 | 2569 | SEQ ID NO: 7516 | 11.27% | 180 |
| 18 | 194 | SEQ ID NO: 7517 | 9.39% | 150 |
| 19 | 1771 | SEQ ID NO: 7518 | 9.39% | 150 |
| 20 | 2488 | SEQ ID NO: 7519 | 9.39% | 150 |

HLA A 0201 - 9 mers

| Maximum possible score using this molecule type | | | | 3925227.1 |
|---|---|---|---|---|
| 1 | 2308 | SEQ ID NO: 7520 | 0.20% | 8144.13515256 |
| 2 | 3729 | SEQ ID NO: 7521 | 0.10% | 4047.23088 |
| 3 | 3574 | SEQ ID NO: 7522 | 0.09% | 3547.4996634 |
| 4 | 3615 | SEQ ID NO: 7523 | 0.06% | 2722.682592 |
| 5 | 3159 | SEQ ID NO: 7524 | 0.05% | 1999.734264 |
| 6 | 2339 | SEQ ID NO: 7525 | 0.03% | 1551.92907744 |
| 7 | 2201 | SEQ ID NO: 7526 | 0.03% | 1521.53694 |
| 8 | 3559 | SEQ ID NO: 7527 | 0.02% | 1174.38939504 |
| 9 | 3085 | SEQ ID NO: 7528 | 0.02% | 1146.296448 |
| 10 | 4070 | SEQ ID NO: 7529 | 0.02% | 970.4103696 |
| 11 | 3708 | SEQ ID NO: 7530 | 0.02% | 958.92888 |
| 12 | 3098 | SEQ ID NO: 7531 | 0.02% | 942.678 |
| 13 | 1362 | SEQ ID NO: 7532 | 0.02% | 900.6984 |
| 14 | 3563 | SEQ ID NO: 7533 | 0.01% | 735.86016 |
| 15 | 3774 | SEQ ID NO: 7534 | 0.01% | 687.655656 |
| 16 | 4242 | SEQ ID NO: 7535 | 0.01% | 685.78272 |
| 17 | 2340 | SEQ ID NO: 7536 | 0.01% | 668.37342936 |
| 18 | 650 | SEQ ID NO: 7537 | 0.01% | 640.1983392 |
| 19 | 3862 | SEQ ID NO: 7538 | 0.01% | 620.57772 |
| 20 | 2860 | SEQ ID NO: 7539 | 0.01% | 607.88448 |

HLA A 0201 - 10 mers

| Maximum possible score using this molecule type | | | | 3925227.1 |
|---|---|---|---|---|
| 1 | 2307 | SEQ ID NO: 7540 | 0.40% | 15915.66281448 |
| 2 | 2201 | SEQ ID NO: 7541 | 0.12% | 4772.09313 |
| 3 | 3558 | SEQ ID NO: 7542 | 0.05% | 2295.04855632 |
| 4 | 1772 | SEQ ID NO: 7543 | 0.04% | 1759.6656 |
| 5 | 3087 | SEQ ID NO: 7544 | 0.03% | 1215.76896 |
| 6 | 2339 | SEQ ID NO: 7545 | 0.02% | 1116.29986272 |
| 7 | 2308 | SEQ ID NO: 7546 | 0.02% | 970.14776112 |
| 8 | 3061 | SEQ ID NO: 7547 | 0.02% | 836.2525104 |
| 9 | 2748 | SEQ ID NO: 7548 | 0.01% | 726.706344 |
| 10 | 3837 | SEQ ID NO: 7549 | 0.01% | 720.8292 |
| 11 | 59 | SEQ ID NO: 7550 | 0.01% | 650.3112 |
| 12 | 2877 | SEQ ID NO: 7551 | 0.01% | 620.22996 |
| 13 | 4114 | SEQ ID NO: 7552 | 0.01% | 559.8936 |
| 14 | 805 | SEQ ID NO: 7553 | 0.01% | 484.4565072 |
| 15 | 1655 | SEQ ID NO: 7554 | 0.01% | 437.48208 |
| 16 | 611 | SEQ ID NO: 7555 | 0.00% | 319.9392 |
| 17 | 1961 | SEQ ID NO: 7556 | 0.00% | 305.94186 |
| 18 | 1223 | SEQ ID NO: 7557 | 0.00% | 289.08792 |
| 19 | 852 | SEQ ID NO: 7558 | 0.00% | 285.67242 |
| 20 | 2139 | SEQ ID NO: 7559 | 0.00% | 284.845869 |

HLA A 1101 - 9 mers

| Maximum possible score using this molecule type | | | | 36 |
|---|---|---|---|---|
| 1 | 4200 | SEQ ID NO: 7560 | 50% | 18 |
| 2 | 281 | SEQ ID NO: 7561 | 25% | 9 |
| 3 | 3236 | SEQ ID NO: 7562 | 25% | 9 |
| 4 | 509 | SEQ ID NO: 7563 | 16.66% | 6 |
| 5 | 848 | SEQ ID NO: 7564 | 16.66% | 6 |
| 6 | 2193 | SEQ ID NO: 7565 | 16.66% | 6 |
| 7 | 3542 | SEQ ID NO: 7566 | 16.66% | 6 |
| 8 | 541 | SEQ ID NO: 7567 | 15% | 5.4 |
| 9 | 1748 | SEQ ID NO: 7568 | 12.5% | 4.5 |
| 10 | 829 | SEQ ID NO: 7569 | 11.11% | 4 |
| 11 | 1149 | SEQ ID NO: 7570 | 11.11% | 4 |
| 12 | 2027 | SEQ ID NO: 7571 | 11.11% | 4 |
| 13 | 2576 | SEQ ID NO: 7572 | 11.11% | 4 |
| 14 | 873 | SEQ ID NO: 7573 | 8.33% | 3 |
| 15 | 2725 | SEQ ID NO: 7574 | 8.33% | 3 |
| 16 | 3541 | SEQ ID NO: 7575 | 8.33% | 3 |
| 17 | 1837 | SEQ ID NO: 7576 | 7.5% | 2.7 |
| 18 | 2475 | SEQ ID NO: 7577 | 7.5% | 2.7 |
| 19 | 2703 | SEQ ID NO: 7578 | 7.5% | 2.7 |
| 20 | 1823 | SEQ ID NO: 7579 | 6.66% | 2.4 |

HLA A 1101 - 10 mers

| Maximum possible score using this molecule type | | | | 36 |
|---|---|---|---|---|
| 1 | 3541 | SEQ ID NO: 7580 | 50% | 18 |
| 2 | 281 | SEQ ID NO: 7581 | 25% | 9 |
| 3 | 1495 | SEQ ID NO: 7582 | 25% | 9 |
| 4 | 2303 | SEQ ID NO: 7583 | 25% | 9 |
| 5 | 2616 | SEQ ID NO: 7584 | 25% | 9 |
| 6 | 48 | SEQ ID NO: 7585 | 16.66% | 6 |
| 7 | 1394 | SEQ ID NO: 7586 | 16.66% | 6 |
| 8 | 1499 | SEQ ID NO: 7587 | 16.66% | 6 |
| 9 | 1862 | SEQ ID NO: 7588 | 16.66% | 6 |
| 10 | 1163 | SEQ ID NO: 7589 | 11.11% | 4 |
| 11 | 4006 | SEQ ID NO: 7590 | 11.11% | 4 |
| 12 | 4344 | SEQ ID NO: 7591 | 11.11% | 4 |
| 13 | 633 | SEQ ID NO: 7592 | 10% | 3.6 |
| 14 | 119 | SEQ ID NO: 7593 | 8.33% | 3 |
| 15 | 1190 | SEQ ID NO: 7594 | 8.33% | 3 |
| 16 | 1195 | SEQ ID NO: 7595 | 8.33% | 3 |
| 17 | 1725 | SEQ ID NO: 7596 | 8.33% | 3 |
| 18 | 2728 | SEQ ID NO: 7597 | 8.33% | 3 |
| 19 | 2895 | SEQ ID NO: 7598 | 8.33% | 3 |
| 20 | 3033 | SEQ ID NO: 7599 | 8.33% | 3 |

HLA B7 - 9 mers

| Maximum possible score using this molecule type | | | | 5400 |
|---|---|---|---|---|
| 1 | 1335 | SEQ ID NO: 7600 | 4.44% | 240 |
| 2 | 2580 | SEQ ID NO: 7601 | 4.44% | 240 |
| 3 | 1703 | SEQ ID NO: 7602 | 3.70% | 200 |
| 4 | 113 | SEQ ID NO: 7603 | 2.22% | 120 |
| 5 | 168 | SEQ ID NO: 7604 | 2.22% | 120 |
| 6 | 2842 | SEQ ID NO: 7605 | 2.22% | 120 |
| 7 | 4027 | SEQ ID NO: 7606 | 2.22% | 120 |
| 8 | 3680 | SEQ ID NO: 7607 | 1.66% | 90 |
| 9 | 2085 | SEQ ID NO: 7608 | 1.48% | 80 |
| 10 | 2492 | SEQ ID NO: 7609 | 1.48% | 80 |
| 11 | 2660 | SEQ ID NO: 7610 | 1.48% | 80 |
| 12 | 2906 | SEQ ID NO: 7611 | 1.48% | 80 |
| 13 | 3346 | SEQ ID NO: 7612 | 1.48% | 80 |
| 14 | 4038 | SEQ ID NO: 7613 | 1.48% | 80 |
| 15 | 1163 | SEQ ID NO: 7614 | 1.11% | 60 |
| 16 | 1457 | SEQ ID NO: 7615 | 1.11% | 60 |
| 17 | 2351 | SEQ ID NO: 7616 | 1.11% | 60 |
| 18 | 2471 | SEQ ID NO: 7617 | 1.11% | 60 |
| 19 | 3499 | SEQ ID NO: 7618 | 1.11% | 60 |
| 20 | 3635 | SEQ ID NO: 7619 | 1.11% | 60 |

HLA B7 - 10 mers

| Maximum possible score using this molecule type | | | | 5400 |
|---|---|---|---|---|
| 1 | 1703 | SEQ ID NO: 7620 | 3.70% | 200 |
| 2 | 17 | SEQ ID NO: 7621 | 2.22% | 120 |
| 3 | 3008 | SEQ ID NO: 7622 | 2.22% | 120 |
| 4 | 4106 | SEQ ID NO: 7623 | 2.22% | 120 |
| 5 | 3450 | SEQ ID NO: 7624 | 1.66% | 90 |
| 6 | 113 | SEQ ID NO: 7625 | 1.48% | 80 |
| 7 | 195 | SEQ ID NO: 7626 | 1.48% | 80 |
| 8 | 307 | SEQ ID NO: 7627 | 1.48% | 80 |
| 9 | 780 | SEQ ID NO: 7628 | 1.48% | 80 |
| 10 | 1000 | SEQ ID NO: 7629 | 1.48% | 80 |
| 11 | 1072 | SEQ ID NO: 7630 | 1.48% | 80 |
| 12 | 1404 | SEQ ID NO: 7631 | 1.48% | 80 |
| 13 | 1980 | SEQ ID NO: 7632 | 1.48% | 80 |
| 14 | 2262 | SEQ ID NO: 7633 | 1.48% | 80 |
| 15 | 2543 | SEQ ID NO: 7634 | 1.48% | 80 |
| 16 | 2906 | SEQ ID NO: 7635 | 1.48% | 80 |
| 17 | 3077 | SEQ ID NO: 7636 | 1.48% | 80 |
| 18 | 3175 | SEQ ID NO: 7637 | 1.48% | 80 |
| 19 | 4195 | SEQ ID NO: 7638 | 1.48% | 80 |
| 20 | 4251 | SEQ ID NO: 7639 | 1.48% | 80 |

TABLE 14
Epitopes for SEQ ID NO: 6040

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 20 | SEQ ID NO: 7640 | 0.04% | 2.25 |
| 2 | 91 | SEQ ID NO: 7641 | 0.01% | 1 |
| 3 | 125 | SEQ ID NO: 7642 | 0.01% | 0.75 |
| 4 | 56 | SEQ ID NO: 7643 | 0.00% | 0.5 |
| 5 | 145 | SEQ ID NO: 7644 | 0.00% | 0.5 |

HLA A1 - 10 mers

| Maximum possible score using this molecule type | | | | 5625 |
|---|---|---|---|---|
| 1 | 20 | SEQ ID NO: 7645 | 0.01% | 0.9 |
| 2 | 56 | SEQ ID NO: 7646 | 0.00% | 0.5 |
| 3 | 71 | SEQ ID NO: 7647 | 0.00% | 0.5 |
| 4 | 144 | SEQ ID NO: 7648 | 0.00% | 0.5 |

HLA A3 - 9 mers

| Maximum possible score using this molecule type | | | | 12150 |
|---|---|---|---|---|
| 1 | 115 | SEQ ID NO: 7649 | 0.24% | 30 |
| 2 | 87 | SEQ ID NO: 7650 | 0.04% | 6 |
| 3 | 80 | SEQ ID NO: 7651 | 0.03% | 4.05 |
| 4 | 125 | SEQ ID NO: 7652 | 0.01% | 1.8 |
| 5 | 39 | SEQ ID NO: 7653 | 0.01% | 1.5 |
| 6 | 56 | SEQ ID NO: 7654 | 0.01% | 1.5 |
| 7 | 135 | SEQ ID NO: 7655 | 0.00% | 1.2 |
| 8 | 91 | SEQ ID NO: 7656 | 0.00% | 1 |
| 9 | 119 | SEQ ID NO: 7657 | 0.00% | 1 |
| 10 | 141 | SEQ ID NO: 7658 | 0.00% | 0.9 |
| 11 | 150 | SEQ ID NO: 7659 | 0.00% | 0.6 |
| 12 | 137 | SEQ ID NO: 7660 | 0.00% | 0.54 |

HLA A3 - 10 mers

| Maximum possible score using this molecule type | | | | 12150 |
|---|---|---|---|---|
| 1 | 36 | SEQ ID NO: 7661 | 0.24% | 30 |
| 2 | 144 | SEQ ID NO: 7662 | 0.06% | 8 |
| 3 | 101 | SEQ ID NO: 7663 | 0.03% | 4 |
| 4 | 99 | SEQ ID NO: 7664 | 0.02% | 3.6 |
| 5 | 80 | SEQ ID NO: 7665 | 0.02% | 2.7 |
| 6 | 125 | SEQ ID NO: 7666 | 0.01% | 1.6875 |
| 7 | 71 | SEQ ID NO: 7667 | 0.01% | 1.5 |
| 8 | 118 | SEQ ID NO: 7668 | 0.01% | 1.5 |
| 9 | 40 | SEQ ID NO: 7669 | 0.01% | 1.35 |
| 10 | 5 | SEQ ID NO: 7670 | 0.00% | 0.9 |
| 11 | 56 | SEQ ID NO: 7671 | 0.00% | 0.9 |
| 12 | 107 | SEQ ID NO: 7672 | 0.00% | 0.6 |
| 13 | 135 | SEQ ID NO: 7673 | 0.00% | 0.6 |
| 14 | 141 | SEQ ID NO: 7674 | 0.00% | 0.6 |
| 15 | 148 | SEQ ID NO: 7675 | 0.00% | 0.6 |
| 16 | 116 | SEQ ID NO: 7676 | 0.00% | 0.5 |

HLA A24 - 9 mers

| Maximum possible score using this molecule type | | | | 1596.672 |
|---|---|---|---|---|
| 1 | 153 | SEQ ID NO: 7677 | 1.05% | 16.8 |
| 2 | 80 | SEQ ID NO: 7678 | 0.75% | 12 |
| 3 | 123 | SEQ ID NO: 7679 | 0.50% | 8 |
| 4 | 137 | SEQ ID NO: 7680 | 0.50% | 8 |
| 5 | 9 | SEQ ID NO: 7681 | 0.45% | 7.2 |
| 6 | 77 | SEQ ID NO: 7682 | 0.45% | 7.2 |
| 7 | 112 | SEQ ID NO: 7683 | 0.45% | 7.2 |
| 8 | 73 | SEQ ID NO: 7684 | 0.41% | 6.6 |
| 9 | 32 | SEQ ID NO: 7685 | 0.37% | 6 |
| 10 | 110 | SEQ ID NO: 7686 | 0.37% | 6 |
| 11 | 140 | SEQ ID NO: 7687 | 0.37% | 6 |
| 12 | 143 | SEQ ID NO: 7688 | 0.37% | 6 |
| 13 | 18 | SEQ ID NO: 7689 | 0.30% | 4.8 |
| 14 | 54 | SEQ ID NO: 7690 | 0.30% | 4.8 |
| 15 | 108 | SEQ ID NO: 7691 | 0.30% | 4.8 |
| 16 | 141 | SEQ ID NO: 7692 | 0.30% | 4.8 |
| 17 | 92 | SEQ ID NO: 7693 | 0.27% | 4.4 |
| 18 | 33 | SEQ ID NO: 7694 | 0.25% | 4 |
| 19 | 49 | SEQ ID NO: 7695 | 0.25% | 4 |
| 20 | 111 | SEQ ID NO: 7696 | 0.25% | 4 |

HLA A24 - 10 mers

| Maximum possible score using this molecule type | | | | 1596.672 |
|---|---|---|---|---|
| 1 | 142 | SEQ ID NO: 7697 | 12.52% | 200 |
| 2 | 110 | SEQ ID NO: 7698 | 0.75% | 12 |
| 3 | 99 | SEQ ID NO: 7699 | 0.50% | 8 |
| 4 | 8 | SEQ ID NO: 7700 | 0.45% | 7.2 |
| 5 | 140 | SEQ ID NO: 7701 | 0.45% | 7.2 |
| 6 | 32 | SEQ ID NO: 7702 | 0.37% | 6 |
| 7 | 17 | SEQ ID NO: 7703 | 0.30% | 4.8 |
| 8 | 53 | SEQ ID NO: 7704 | 0.30% | 4.8 |
| 9 | 76 | SEQ ID NO: 7705 | 0.30% | 4.8 |
| 10 | 107 | SEQ ID NO: 7706 | 0.30% | 4.8 |
| 11 | 111 | SEQ ID NO: 7707 | 0.30% | 4.8 |
| 12 | 72 | SEQ ID NO: 7708 | 0.27% | 4.4 |
| 13 | 91 | SEQ ID NO: 7709 | 0.27% | 4.4 |
| 14 | 31 | SEQ ID NO: 7710 | 0.25% | 4 |
| 15 | 127 | SEQ ID NO: 7711 | 0.25% | 4 |
| 16 | 139 | SEQ ID NO: 7712 | 0.25% | 4 |
| 17 | 80 | SEQ ID NO: 7713 | 0.22% | 3.6 |
| 18 | 38 | SEQ ID NO: 7714 | 0.18% | 3 |
| 19 | 118 | SEQ ID NO: 7715 | 0.18% | 3 |
| 20 | 49 | SEQ ID NO: 7716 | 0.12% | 2 |

HLA A 0201 - 9 mers

| Maximum possible score using this molecule type | | | | 3925227.1 |
|---|---|---|---|---|
| 1 | 80 | SEQ ID NO: 7717 | 0.00% | 171.96732 |
| 2 | 147 | SEQ ID NO: 7718 | 0.00% | 51.46848 |
| 3 | 143 | SEQ ID NO: 7719 | 0.00% | 11.6146182 |
| 4 | 56 | SEQ ID NO: 7720 | 0.00% | 11.304684 |
| 5 | 10 | SEQ ID NO: 7721 | 0.00% | 10.34586 |
| 6 | 6 | SEQ ID NO: 7722 | 0.00% | 6.56830734 |
| 7 | 26 | SEQ ID NO: 7723 | 0.00% | 6.07614 |
| 8 | 141 | SEQ ID NO: 7724 | 0.00% | 5.981472 |
| 9 | 148 | SEQ ID NO: 7725 | 0.00% | 5.194044 |
| 10 | 9 | SEQ ID NO: 7726 | 0.00% | 4.299183 |
| 11 | 137 | SEQ ID NO: 7727 | 0.00% | 4.299183 |
| 12 | 130 | SEQ ID NO: 7728 | 0.00% | 4.138344 |
| 13 | 84 | SEQ ID NO: 7729 | 0.00% | 3.42792 |
| 14 | 27 | SEQ ID NO: 7730 | 0.00% | 3.383484 |
| 15 | 2 | SEQ ID NO: 7731 | 0.00% | 3.381 |
| 16 | 62 | SEQ ID NO: 7732 | 0.00% | 3.251556 |
| 17 | 23 | SEQ ID NO: 7733 | 0.00% | 2.9542005 |
| 18 | 99 | SEQ ID NO: 7734 | 0.00% | 1.982232 |
| 19 | 33 | SEQ ID NO: 7735 | 0.00% | 1.86921 |
| 20 | 111 | SEQ ID NO: 7736 | 0.00% | 1.76402985 |

HLA A 0201 - 10 mers

| Maximum possible score using this molecule type | | | | 3925227.1 |
|---|---|---|---|---|
| 1 | 5 | SEQ ID NO: 7737 | 0.00% | 159.9696 |
| 2 | 25 | SEQ ID NO: 7738 | 0.00% | 69.552 |
| 3 | 80 | SEQ ID NO: 7739 | 0.00% | 36.5148 |
| 4 | 107 | SEQ ID NO: 7740 | 0.00% | 21.3624 |
| 5 | 148 | SEQ ID NO: 7741 | 0.00% | 17.73576 |
| 6 | 61 | SEQ ID NO: 7742 | 0.00% | 13.9104 |
| 7 | 147 | SEQ ID NO: 7743 | 0.00% | 11.304684 |
| 8 | 53 | SEQ ID NO: 7744 | 0.00% | 8.230458 |
| 9 | 17 | SEQ ID NO: 7745 | 0.00% | 7.3086111 |
| 10 | 110 | SEQ ID NO: 7746 | 0.00% | 6.174104475 |
| 11 | 9 | SEQ ID NO: 7747 | 000% | 6.0858 |
| 12 | 99 | SEQ ID NO: 7748 | 0.00% | 5.6823984 |
| 13 | 2 | SEQ ID NO: 7749 | 0.00% | 3.188283 |
| 14 | 41 | SEQ ID NO: 7750 | 0.00% | 12.206413 |
| 15 | 135 | SEQ ID NO: 7751 | 0.00% | 12.076624 |
| 16 | 76 | SEQ ID NO: 7752 | 0.00% | 2.005692 |
| 17 | 23 | SEQ ID NO: 7753 | 0.00% | 1.798209 |
| 18 | 40 | SEQ ID NO: 7754 | 0.00% | 1.68996456 |
| 19 | 39 | SEQ ID NO: 7755 | 0.00% | 1.516482 |
| 20 | 118 | SEQ ID NO: 7756 | 0.00% | 1.2683304 |

HLA A 1101 - 9 mers

| Maximum possible score using this molecule type | | | | 36 |
|---|---|---|---|---|
| 1 | 91 | SEQ ID NO: 7757 | 2.77% | 1 |

HLA A 1101 - 10 mers

| Maximum possible score using this molecule type | | | | 36 |
|---|---|---|---|---|
| 1 | 101 | SEQ ID NO: 7758 | 33.33% | 12 |
| 2 | 71 | SEQ ID NO: 7759 | 2.77% | 1 |
| 3 | 90 | SEQ ID NO: 7760 | 1.66% | 0.6 |

HLA B7 - 9 mers

| Maximum possible score using this molecule type | | | | 5400 |
|---|---|---|---|---|
| 1 | 49 | SEQ ID NO: 7761 | 2.22% | 120 |
| 2 | 9 | SEQ ID NO: 7762 | 1.11% | 60 |
| 3 | 73 | SEQ ID NO: 7763 | 0.66% | 36 |
| 4 | 33 | SEQ ID NO: 7764 | 0.37% | 20 |
| 5 | 137 | SEQ ID NO: 7765 | 0.37% | 20 |
| 6 | 141 | SEQ ID NO: 7766 | 0.37% | 20 |
| 7 | 77 | SEQ ID NO: 7767 | 0.22% | 12 |
| 8 | 112 | SEQ ID NO: 7768 | 0.22% | 12 |
| 9 | 143 | SEQ ID NO: 7769 | 0.22% | 12 |
| 10 | 81 | SEQ ID NO: 7770 | 0.14% | 8 |
| 11 | 13 | SEQ ID NO: 7771 | 0.09% | 5 |
| 12 | 69 | SEQ ID NO: 7772 | 0.09% | 5 |
| 13 | 18 | SEQ ID NO: 7773 | 0.07% | 4 |
| 14 | 32 | SEQ ID NO: 7774 | 0.07% | 4 |
| 15 | 54 | SEQ ID NO: 7775 | 0.07% | 4 |
| 16 | 80 | SEQ ID NO: 7776 | 0.07% | 4 |
| 17 | 92 | SEQ ID NO: 7777 | 0.07% | 4 |
| 18 | 108 | SEQ ID NO: 7778 | 0.07% | 4 |
| 19 | 111 | SEQ ID NO: 7779 | 0.07% | 4 |
| 20 | 123 | SEQ ID NO: 7780 | 0.07% | 4 |

HLA B7 - 10 mers

| Maximum possible score using this molecule type | | | | 5400 |
|---|---|---|---|---|
| 1 | 99 | SEQ ID NO: 7781 | 0.74% | 40 |
| 2 | 17 | SEQ ID NO: 7782 | 0.37% | 20 |
| 3 | 8 | SEQ ID NO: 7783 | 0.22% | 12 |
| 4 | 72 | SEQ ID NO: 7784 | 0.22% | 12 |
| 5 | 91 | SEQ ID NO: 7785 | 0.22% | 12 |
| 6 | 127 | SEQ ID NO: 7786 | 0.11% | 6 |
| 7 | 31 | SEQ ID NO: 7787 | 0.07% | 4 |
| 8 | 32 | SEQ ID NO: 7788 | 0.07% | 4 |
| 9 | 53 | SEQ ID NO: 7789 | 0.07% | 4 |
| 10 | 76 | SEQ ID NO: 7790 | 0.07% | 4 |
| 11 | 107 | SEQ ID NO: 7791 | 0.07% | 4 |
| 12 | 110 | SEQ ID NO: 7792 | 0.07% | 4 |
| 13 | 111 | SEQ ID NO: 7793 | 0.07% | 4 |
| 14 | 140 | SEQ ID NO: 7794 | 0.07% | 4 |
| 15 | 9 | SEQ ID NO: 7795 | 0.05% | 3 |
| 16 | 19 | SEQ ID NO: 7796 | 0.05% | 3 |
| 17 | 33 | SEQ ID NO: 7797 | 0.03% | 2 |
| 18 | 93 | SEQ ID NO: 7798 | 0.03% | 2 |
| 19 | 102 | SEQ ID NO: 7799 | 0.03% | 2 |
| 20 | 129 | SEQ ID NO: 7800 | 0.02% | 1.5 |

TABLE 15
Epitopes for SEQ ID NO: 6041

| | | | | |
|---|---|---|---|---|
| | | | % of | |
| | Start | | max. | |
| Rank | position | Sequence | score | Score |

HLA B7 - 10 mers

| Maximum possible score using | | | | 5625 |
|---|---|---|---|---|
| this molecule type | | | | |
| 1 | 1818 | SEQ ID NO: 7801 | 1.6% | 90 |
| 2 | 373 | SEQ ID NO: 7802 | 1.33% | 75 |
| 3 | 681 | SEQ ID NO: 7803 | 1.33% | 75 |
| 4 | 74 | SEQ ID NO: 7804 | 0.88% | 50 |
| 5 | 786 | SEQ ID NO: 7805 | 0.88% | 50 |
| 6 | 1495 | SEQ ID NO: 7806 | 0.88% | 50 |
| 7 | 88 | SEQ ID NO: 7807 | 0.8% | 45 |
| 8 | 357 | SEQ ID NO: 7808 | 0.8% | 45 |
| 9 | 1271 | SEQ ID NO: 7809 | 0.8% | 45 |
| 10 | 1799 | SEQ ID NO: 7810 | 0.8% | 45 |
| 11 | 1393 | SEQ ID NO: 7811 | 0.48% | 27 |
| 12 | 386 | SEQ ID NO: 7812 | 0.44% | 25 |
| 13 | 2304 | SEQ ID NO: 7813 | 0.44% | 25 |
| 14 | 198 | SEQ ID NO: 7814 | 0.4% | 22.5 |
| 15 | 840 | SEQ ID NO: 7815 | 0.4% | 22.5 |
| 16 | 2359 | SEQ ID NO: 7816 | 0.4% | 22.5 |
| 17 | 1194 | SEQ ID NO: 7817 | 0.32% | 18 |
| 18 | 1546 | SEQ ID NO: 7818 | 0.32% | 18 |
| 19 | 2200 | SEQ ID NO: 7819 | 0.22% | 12.5 |
| 20 | 996 | SEQ ID NO: 7820 | 0.2% | 11.25 |

HLA A1 - 9 mers

| Maximum possible score using | | | | 5625 |
|---|---|---|---|---|
| this molecule type | | | | |
| 1 | 995 | SEQ ID NO: 7821 | 10% | 562.5 |
| 2 | 1303 | SEQ ID NO: 7822 | 2.22% | 125 |
| 3 | 1582 | SEQ ID NO: 7823 | 2% | 112.5 |
| 4 | 1456 | SEQ ID NO: 7824 | 1.6% | 90 |
| 5 | 772 | SEQ ID NO: 7825 | 1.11% | 62.5 |
| 6 | 181 | SEQ ID NO: 7826 | 0.88% | 50 |
| 7 | 632 | SEQ ID NO: 7827 | 0.88% | 50 |
| 8 | 2281 | SEQ ID NO: 7828 | 0.88% | 50 |
| 9 | 1586 | SEQ ID NO: 7829 | 0.8% | 45 |
| 10 | 2109 | SEQ ID NO: 7830 | 0.8% | 45 |
| 11 | 745 | SEQ ID NO: 7831 | 0.55% | 31.25 |
| 12 | 1916 | SEQ ID NO: 7832 | 0.53% | 30 |
| 13 | 966 | SEQ ID NO: 7833 | 0.44% | 25 |
| 14 | 1387 | SEQ ID NO: 7834 | 0.44% | 25 |
| 15 | 2263 | SEQ ID NO: 7835 | 0.44% | 25 |
| 16 | 2457 | SEQ ID NO: 7836 | 0.26% | 15 |
| 17 | 1057 | SEQ ID NO: 7837 | 0.22% | 12.5 |
| 18 | 2562 | SEQ ID NO: 7838 | 0.22% | 12.5 |
| 19 | 74 | SEQ ID NO: 7839 | 0.17% | 10 |
| 20 | 298 | SEQ ID NO: 7840 | 0.17% | 10 |

HLA A1 - 10 mers

| Maximum possible score using | | | | 12150 |
|---|---|---|---|---|
| this molecule type | | | | |
| 1 | 536 | SEQ ID NO: 7841 | 3.33% | 405 |
| 2 | 986 | SEQ ID NO: 7842 | 2.46% | 300 |
| 3 | 805 | SEQ ID NO: 7843 | 1.64% | 200 |
| 4 | 2345 | SEQ ID NO: 7844 | 1.48% | 180 |
| 5 | 2481 | SEQ ID NO: 7845 | 0.55% | 67.5 |
| 6 | 204 | SEQ ID NO: 7846 | 0.49% | 60 |
| 7 | 895 | SEQ ID NO: 7847 | 0.44% | 54 |
| 8 | 1512 | SEQ ID NO: 7848 | 0.44% | 54 |
| 9 | 2491 | SEQ ID NO: 7849 | 0.37% | 45 |
| 10 | 436 | SEQ ID NO: 7850 | 0.32% | 40 |
| 11 | 917 | SEQ ID NO: 7851 | 0.32% | 40 |
| 12 | 1176 | SEQ ID NO: 7852 | 0.32% | 40 |
| 13 | 1517 | SEQ ID NO: 7853 | 0.29% | 36 |
| 14 | 466 | SEQ ID NO: 7854 | 0.24% | 30 |
| 15 | 1784 | SEQ ID NO: 7855 | 0.24% | 30 |
| 16 | 2039 | SEQ ID NO: 7856 | 0.24% | 30 |
| 17 | 2124 | SEQ ID NO: 7857 | 0.24% | 30 |
| 18 | 1049 | SEQ ID NO: 7858 | 0.22% | 27 |
| 19 | 2200 | SEQ ID NO: 7859 | 0.22% | 27 |
| 20 | 2598 | SEQ ID NO: 7860 | 0.22% | 27 |

HLA A3 - 9 mers

| Maximum possible score using | | | | 12150 |
|---|---|---|---|---|
| this molecule type | | | | |
| 1 | 392 | SEQ ID NO: 7861 | 2.46% | 300 |
| 2 | 2230 | SEQ ID NO: 7862 | 1.48% | 180 |
| 3 | 590 | SEQ ID NO: 7863 | 1.11% | 135 |
| 4 | 697 | SEQ ID NO: 7864 | 1.11% | 135 |
| 5 | 919 | SEQ ID NO: 7865 | 0.74% | 90 |
| 6 | 1354 | SEQ ID NO: 7866 | 0.74% | 90 |
| 7 | 1430 | SEQ ID NO: 7867 | 0.74% | 90 |
| 8 | 2534 | SEQ ID NO: 7868 | 0.74% | 90 |
| 9 | 202 | SEQ ID NO: 7869 | 0.49% | 60 |
| 10 | 488 | SEQ ID NO: 7870 | 0.49% | 60 |
| 11 | 922 | SEQ ID NO: 7871 | 0.49% | 60 |
| 12 | 1735 | SEQ ID NO: 7872 | 0.49% | 60 |
| 13 | 2281 | SEQ ID NO: 7873 | 0.49% | 60 |
| 14 | 1894 | SEQ ID NO: 7874 | 0.44% | 54 |
| 15 | 2552 | SEQ ID NO: 7875 | 0.44% | 54 |
| 16 | 555 | SEQ ID NO: 7876 | 0.37% | 45 |
| 17 | 1134 | SEQ ID NO: 7877 | 0.37% | 45 |
| 18 | 1149 | SEQ ID NO: 7878 | 0.29% | 36 |
| 19 | 283 | SEQ ID NO: 7879 | 0.24% | 30 |
| 20 | 917 | SEQ ID NO: 7880 | 0.24% | 30 |

HLA A3 - 10 mers

| Maximum possible score using | | | | 1596.672 |
|---|---|---|---|---|
| this molecule type | | | | |
| 1 | 2375 | SEQ ID NO: 7881 | 36.07% | 576 |
| 2 | 1751 | SEQ ID NO: 7882 | 28.93% | 462 |
| 3 | 195 | SEQ ID NO: 7883 | 25.05% | 400 |
| 4 | 2306 | SEQ ID NO: 7884 | 21.04% | 336 |
| 5 | 806 | SEQ ID NO: 7885 | 20.66% | 330 |
| 6 | 1252 | SEQ ID NO: 7886 | 18.78% | 300 |
| 7 | 160 | SEQ ID NO: 7887 | 15.03% | 240 |
| 8 | 517 | SEQ ID NO: 7888 | 15.03% | 240 |
| 9 | 375 | SEQ ID NO: 7889 | 12.52% | 200 |
| 10 | 1275 | SEQ ID NO: 7890 | 12.52% | 200 |
| 11 | 2175 | SEQ ID NO: 7891 | 12.52% | 200 |
| 12 | 2207 | SEQ ID NO: 7892 | 12.52% | 200 |
| 13 | 2343 | SEQ ID NO: 7893 | 12.52% | 200 |
| 14 | 443 | SEQ ID NO: 7894 | 11.27% | 180 |
| 15 | 668 | SEQ ID NO: 7895 | 7.51% | 120 |
| 16 | 1825 | SEQ ID NO: 7896 | 6.88% | 110 |
| 17 | 1690 | SEQ ID NO: 7897 | 4.69% | 75 |
| 18 | 159 | SEQ ID NO: 7898 | 3.75% | 60 |
| 19 | 2550 | SEQ ID NO: 7899 | 3.75% | 60 |
| 20 | 1949 | SEQ ID NO: 7900 | 3.38% | 54 |

HLA A24 - 9 mers

| Maximum possible score using | | | | 1596.672 |
|---|---|---|---|---|
| this molecule type | | | | |
| 1 | 641 | SEQ ID NO: 7901 | 45.09% | 720 |
| 2 | 809 | SEQ ID NO: 7902 | 24.80% | 396 |
| 3 | 1209 | SEQ ID NO: 7903 | 22.54% | 360 |
| 4 | 216 | SEQ ID NO: 7904 | 18.03% | 288 |
| 5 | 159 | SEQ ID NO: 7905 | 15.03% | 240 |
| 6 | 528 | SEQ ID NO: 7906 | 15.03% | 240 |
| 7 | 799 | SEQ ID NO: 7907 | 15.03% | 240 |
| 8 | 1436 | SEQ ID NO: 7908 | 15.03% | 240 |
| 9 | 2219 | SEQ ID NO: 7909 | 15.03% | 240 |
| 10 | 1065 | SEQ ID NO: 7910 | 13.77% | 220 |
| 11 | 1953 | SEQ ID NO: 7911 | 13.15% | 210 |
| 12 | 1966 | SEQ ID NO: 7912 | 12.52% | 200 |
| 13 | 2600 | SEQ ID NO: 7913 | 12.52% | 200 |
| 14 | 71 | SEQ ID NO: 7914 | 9.39% | 150 |
| 15 | 380 | SEQ ID NO: 7915 | 9.39% | 150 |
| 16 | 1989 | SEQ ID NO: 7916 | 9.39% | 150 |
| 17 | 342 | SEQ ID NO: 7917 | 8.76% | 140 |
| 18 | 1071 | SEQ ID NO: 7918 | 8.76% | 140 |
| 19 | 2570 | SEQ ID NO: 7919 | 6.88% | 110 |
| 20 | 2550 | SEQ ID NO: 7920 | 6.26% | 100 |

HLA A24 - 10 mers

| Maximum possible score using | | | | 3925227.1 |
|---|---|---|---|---|
| this molecule type | | | | |
| 1 | 1632 | SEQ ID NO: 7921 | 0.09% | 3607.31448 |
| 2 | 1640 | SEQ ID NO: 7922 | 0.04% | 1748.2560912 |
| 3 | 1776 | SEQ ID NO: 7923 | 0.03% | 1492.58592 |
| 4 | 2512 | SEQ ID NO: 7924 | 0.03% | 1434.16845 |
| 5 | 1073 | SEQ ID NO: 7925 | 0.03% | 1338.876 |
| 6 | 230 | SEQ ID NO: 7926 | 0.01% | 685.78272 |
| 7 | 1001 | SEQ ID NO: 7927 | 0.01% | 559.8936 |
| 8 | 716 | SEQ ID NO: 7928 | 0.01% | 558.27486 |
| 9 | 2280 | SEQ ID NO: 7929 | 0.01% | 511.19781048 |
| 10 | 590 | SEQ ID NO: 7930 | 0.01% | 469.6692 |
| 11 | 664 | SEQ ID NO: 7931 | 0.01% | 442.076389524 |
| 12 | 1094 | SEQ ID NO: 7932 | 0.00% | 382.536 |
| 13 | 1735 | SEQ ID NO: 7933 | 0.00% | 382.536 |
| 14 | 1625 | SEQ ID NO: 7934 | 0.00% | 342.4606344 |
| 15 | 1974 | SEQ ID NO: 7935 | 0.00% | 336.885048 |
| 16 | 2382 | SEQ ID NO: 7936 | 0.00% | 319.9392 |
| 17 | 2417 | SEQ ID NO: 7937 | 0.00% | 319.9392 |
| 18 | 744 | SEQ ID NO: 7938 | 0.00% | 256.416670125 |
| 19 | 108 | SEQ ID NO: 7939 | 0.00% | 232.52724 |
| 20 | 390 | SEQ ID NO: 7940 | 0.00% | 228.0411084 |

HLA A 0201 - 9 mers

| Maximum possible score using | | | | 3925227.1 |
|---|---|---|---|---|
| this molecule type | | | | |
| 1 | 2511 | SEQ ID NO: 7941 | 0.38% | 15126.90795 |
| 2 | 1608 | SEQ ID NO: 7942 | 0.05% | 2049.4656 |
| 3 | 2572 | SEQ ID NO: 7943 | 0.04% | 1879.5921264 |
| 4 | 255 | SEQ ID NO: 7944 | 0.03% | 1566.6522795 |
| 5 | 895 | SEQ ID NO: 7945 | 0.03% | 1338.876 |
| 6 | 1171 | SEQ ID NO: 7946 | 0.02% | 1107.960876 |
| 7 | 1691 | SEQ ID NO: 7947 | 0.01% | 782.95521024 |
| 8 | 20 | SEQ ID NO: 7948 | 0.01% | 549.9372312 |
| 9 | 1632 | SEQ ID NO: 7949 | 0.01% | 479.041993296 |
| 10 | 2280 | SEQ ID NO: 7950 | 0.01% | 472.418344576987 |
| 11 | 1963 | SEQ ID NO: 7951 | 0.00% | 358.73928 |
| 12 | 1955 | SEQ ID NO: 7952 | 0.00% | 331.093464 |
| 13 | 741 | SEQ ID NO: 7953 | 0.00% | 318.652488 |
| 14 | 523 | SEQ ID NO: 7954 | 0.00% | 278.7876 |
| 15 | 1073 | SEQ ID NO: 7955 | 0.00% | 266.6988828 |
| 16 | 2489 | SEQ ID NO: 7956 | 0.00% | 243.432 |
| 17 | 777 | SEQ ID NO: 7957 | 0.00% | 218.5730664 |
| 18 | 1737 | SEQ ID NO: 7958 | 0.00% | 218.0785572 |
| 19 | 589 | SEQ ID NO: 7959 | 0.00% | 210.538251 |
| 20 | 229 | SEQ ID NO: 7960 | 0.00% | 205.230564 |

HLA A 0201 - 10 mers

| Maximum possible score using | | | | 36 |
|---|---|---|---|---|
| this molecule type | | | | |
| 1 | 2337 | SEQ ID NO: 7961 | 33.33% | 12 |
| 2 | 2156 | SEQ ID NO: 7962 | 25% | 9 |
| 3 | 492 | SEQ ID NO: 7963 | 20% | 7.2 |
| 4 | 18 | SEQ ID NO: 7964 | 16.66% | 6 |
| 5 | 332 | SEQ ID NO: 7965 | 16.66% | 6 |
| 6 | 415 | SEQ ID NO: 7966 | 16.66% | 6 |
| 7 | 2479 | SEQ ID NO: 7967 | 16.66% | 6 |
| 8 | 1495 | SEQ ID NO: 7968 | 11.11% | 4 |
| 9 | 2035 | SEQ ID NO: 7969 | 11.11% | 4 |
| 10 | 1349 | SEQ ID NO: 7970 | 10% | 3.6 |
| 11 | 1194 | SEQ ID NO: 7971 | 8.33% | 3 |
| 12 | 1648 | SEQ ID NO: 7972 | 8.33% | 3 |
| 13 | 96 | SEQ ID NO: 7973 | 6.66% | 2.4 |
| 14 | 764 | SEQ ID NO: 7974 | 6.66% | 2.4 |
| 15 | 986 | SEQ ID NO: 7975 | 6.66% | 2.4 |
| 16 | 2345 | SEQ ID NO: 7976 | 6.66% | 2.4 |
| 17 | 698 | SEQ ID NO: 7977 | 5.55% | 2 |
| 18 | 1355 | SEQ ID NO: 7978 | 5.55% | 2 |
| 19 | 1987 | SEQ ID NO: 7979 | 5.55% | 2 |
| 20 | 2085 | SEQ ID NO: 7980 | 5.55% | 2 |

HLA A 1101 - 9 mers

| Maximum possible score using | | | | 36 |
|---|---|---|---|---|
| this molecule type | | | | |
| 1 | 2083 | SEQ ID NO: 7981 | 33.33% | 12 |
| 2 | 2123 | SEQ ID NO: 7982 | 25% | 9 |
| 3 | 2147 | SEQ ID NO: 7983 | 16.66% | 6 |
| 4 | 331 | SEQ ID NO: 7984 | 12.5% | 4.5 |
| 5 | 1035 | SEQ ID NO: 7985 | 11.11% | 4 |
| 6 | 1064 | SEQ ID NO: 7986 | 11.11% | 4 |
| 7 | 2154 | SEQ ID NO: 7987 | 11.11% | 4 |
| 8 | 1048 | SEQ ID NO: 7988 | 7.5% | 2.7 |
| 9 | 202 | SEQ ID NO: 7989 | 6.66% | 2.4 |
| 10 | 721 | SEQ ID NO: 7990 | 6.66% | 2.4 |
| 11 | 2109 | SEQ ID NO: 7991 | 6.66% | 2.4 |
| 12 | 2230 | SEQ ID NO: 7992 | 6.66% | 2.4 |
| 13 | 1306 | SEQ ID NO: 7993 | 5.55% | 2 |
| 14 | 1622 | SEQ ID NO: 7994 | 5.55% | 2 |
| 15 | 1772 | SEQ ID NO: 7995 | 5.55% | 2 |
| 16 | 1796 | SEQ ID NO: 7996 | 5.55% | 2 |
| 17 | 186 | SEQ ID NO: 7997 | 5% | 1.8 |
| 18 | 414 | SEQ ID NO: 7998 | 5% | 1.8 |
| 19 | 697 | SEQ ID NO: 7999 | 5% | 1.8 |
| 20 | 1175 | SEQ ID NO: 8000 | 5% | 1.8 |

HLA A 1101 - 10 mers

| Maximum possible score using | | | | 5400 |
|---|---|---|---|---|
| this molecule type | | | | |
| 1 | 1447 | SEQ ID NO: 8001 | 14.81% | 800 |
| 2 | 642 | SEQ ID NO: 8002 | 3.70% | 200 |
| 3 | 34 | SEQ ID NO: 8003 | 2.22% | 120 |
| 4 | 186 | SEQ ID NO: 8004 | 1.48% | 80 |
| 5 | 244 | SEQ ID NO: 8005 | 1.48% | 80 |
| 6 | 459 | SEQ ID NO: 8006 | 1.48% | 80 |
| 7 | 1475 | SEQ ID NO: 8007 | 1.48% | 80 |
| 8 | 1867 | SEQ ID NO: 8008 | 1.48% | 80 |
| 9 | 2032 | SEQ ID NO: 8009 | 1.48% | 80 |
| 10 | 2047 | SEQ ID NO: 8010 | 1.48% | 80 |
| 11 | 2335 | SEQ ID NO: 8011 | 1.48% | 80 |
| 12 | 622 | SEQ ID NO: 8012 | 1.11% | 60 |
| 13 | 1375 | SEQ ID NO: 8013 | 1.11% | 60 |
| 14 | 1617 | SEQ ID NO: 8014 | 0.92% | 50 |
| 15 | 1023 | SEQ ID NO: 8015 | 0.83% | 45 |
| 16 | 286 | SEQ ID NO: 8016 | 0.74% | 40 |
| 17 | 490 | SEQ ID NO: 8017 | 0.74% | 40 |
| 18 | 810 | SEQ ID NO: 8018 | 0.74% | 40 |
| 19 | 1420 | SEQ ID NO: 8019 | 0.74% | 40 |
| 20 | 1854 | SEQ ID NO: 8020 | 0.74% | 40 |

HLA B7 - 9 mers

| Maximum possible score using | | | | 5400 |
|---|---|---|---|---|
| this molecule type | | | | |
| 1 | 1617 | SEQ ID NO: 8021 | 3.70% | 200 |
| 2 | 752 | SEQ ID NO: 8022 | 2.22% | 120 |
| 3 | 1552 | SEQ ID NO: 8023 | 2.22% | 120 |
| 4 | 154 | SEQ ID NO: 8024 | 1.48% | 80 |
| 5 | 165 | SEQ ID NO: 8025 | 1.48% | 80 |
| 6 | 383 | SEQ ID NO: 8026 | 1.48% | 80 |
| 7 | 1501 | SEQ ID NO: 8027 | 1.48% | 80 |
| 8 | 2093 | SEQ ID NO: 8028 | 1.48% | 80 |
| 9 | 2564 | SEQ ID NO: 8029 | 1.48% | 80 |
| 10 | 622 | SEQ ID NO: 8030 | 1.11% | 60 |
| 11 | 1086 | SEQ ID NO: 8031 | 1.11% | 60 |
| 12 | 1262 | SEQ ID NO: 8032 | 1.11% | 60 |
| 13 | 1556 | SEQ ID NO: 8033 | 1.11% | 60 |
| 14 | 845 | SEQ ID NO: 8034 | 1% | 54 |
| 15 | 286 | SEQ ID NO: 8035 | 0.74% | 40 |
| 16 | 490 | SEQ ID NO: 8036 | 0.74% | 40 |
| 17 | 552 | SEQ ID NO: 8037 | 0.74% | 40 |
| 18 | 1858 | SEQ ID NO: 8038 | 0.74% | 40 |
| 19 | 2107 | SEQ ID NO: 8039 | 0.74% | 40 |
| 20 | 2582 | SEQ ID NO: 8040 | 0.74% | 40 |

TABLE 16
Epitopes for SEQ ID NO: 6042

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 846 | SEQ ID NO: 8041 | 2.22% | 125 |
| 2 | 798 | SEQ ID NO: 8042 | 1.6% | 90 |
| 3 | 787 | SEQ ID NO: 8043 | 0.88% | 50 |
| 4 | 1178 | SEQ ID NO: 8044 | 0.88% | 50 |
| 5 | 637 | SEQ ID NO: 8045 | 0.8% | 45 |
| 6 | 557 | SEQ ID NO: 8046 | 0.44% | 25 |
| 7 | 1020 | SEQ ID NO: 8047 | 0.44% | 25 |
| 8 | 282 | SEQ ID NO: 8048 | 0.32% | 18 |
| 9 | 1241 | SEQ ID NO: 8049 | 0.24% | 13.5 |
| 10 | 466 | SEQ ID NO: 8050 | 0.22% | 12.5 |
| 11 | 727 | SEQ ID NO: 8051 | 0.2% | 11.25 |
| 12 | 706 | SEQ ID NO: 8052 | 0.17% | 10 |
| 13 | 324 | SEQ ID NO: 8053 | 0.16% | 9 |
| 14 | 752 | SEQ ID NO: 8054 | 0.16% | 9 |
| 15 | 54 | SEQ ID NO: 8055 | 0.13% | 7.5 |
| 16 | 554 | SEQ ID NO: 8056 | 0.13% | 7.5 |
| 17 | 590 | SEQ ID NO: 8057 | 0.12% | 6.75 |
| 18 | 569 | SEQ ID NO: 8058 | 0.08% | 5 |
| 19 | 613 | SEQ ID NO: 8059 | 0.08% | 5 |
| 20 | 90 | SEQ ID NO: 8060 | 0.08% | 4.5 |

HLA A1 - 10 mers

| Maximum possible score using this molecule type | | | | 5625 |
|---|---|---|---|---|
| 1 | 1241 | SEQ ID NO: 8061 | 4.8% | 270 |
| 2 | 967 | SEQ ID NO: 8062 | 0.8% | 45 |
| 3 | 1010 | SEQ ID NO: 8063 | 0.48% | 27 |
| 4 | 426 | SEQ ID NO: 8064 | 0.44% | 25 |
| 5 | 809 | SEQ ID NO: 8065 | 0.44% | 25 |
| 6 | 1178 | SEQ ID NO: 8066 | 0.44% | 25 |
| 7 | 787 | SEQ ID NO: 8067 | 0.22% | 12.5 |
| 8 | 958 | SEQ ID NO: 8068 | 0.22% | 12.5 |
| 9 | 727 | SEQ ID NO: 8069 | 0.2% | 11.25 |
| 10 | 610 | SEQ ID NO: 8070 | 0.17% | 10 |
| 11 | 12 | SEQ ID NO: 8071 | 0.13% | 7.5 |
| 12 | 1181 | SEQ ID NO: 8072 | 0.12% | 6.75 |
| 13 | 373 | SEQ ID NO: 8073 | 0.11% | 6.25 |
| 14 | 602 | SEQ ID NO: 8074 | 0.11% | 6.25 |
| 15 | 20 | SEQ ID NO: 8075 | 0.04% | 2.5 |
| 16 | 32 | SEQ ID NO: 8076 | 0.04% | 2.5 |
| 17 | 53 | SEQ ID NO: 8077 | 0.04% | 2.5 |
| 18 | 400 | SEQ ID NO: 8078 | 0.04% | 2.5 |
| 19 | 557 | SEQ ID NO: 8079 | 0.04% | 2.5 |
| 20 | 667 | SEQ ID NO: 8080 | 0.04% | 2.5 |

HLA A3 - 9 mers

| Maximum possible score using this molecule type | | | | 12150 |
|---|---|---|---|---|
| 1 | 768 | SEQ ID NO: 8081 | 0.82% | 100 |
| 2 | 808 | SEQ ID NO: 8082 | 0.49% | 60 |
| 3 | 85 | SEQ ID NO: 8083 | 0.24% | 30 |
| 4 | 663 | SEQ ID NO: 8084 | 0.24% | 30 |
| 5 | 1245 | SEQ ID NO: 8085 | 0.14% | 18 |
| 6 | 288 | SEQ ID NO: 8086 | 0.09% | 12 |
| 7 | 50 | SEQ ID NO: 8087 | 0.08% | 10 |
| 8 | 320 | SEQ ID NO: 8088 | 0.07% | 9 |
| 9 | 402 | SEQ ID NO: 8089 | 0.07% | 9 |
| 10 | 798 | SEQ ID NO: 8090 | 0.07% | 9 |
| 11 | 902 | SEQ ID NO: 8091 | 0.06% | 8.1 |
| 12 | 364 | SEQ ID NO: 8092 | 0.05% | 6.75 |
| 13 | 297 | SEQ ID NO: 8093 | 0.04% | 6 |
| 14 | 992 | SEQ ID NO: 8094 | 0.04% | 6 |
| 15 | 38 | SEQ ID NO: 8095 | 0.03% | 4.5 |
| 16 | 249 | SEQ ID NO: 8096 | 0.03% | 4.5 |
| 17 | 706 | SEQ ID NO: 8097 | 0.03% | 4.05 |
| 18 | 1204 | SEQ ID NO: 8098 | 0.03% | 4.05 |
| 19 | 1178 | SEQ ID NO: 8099 | 0.03% | 4 |
| 20 | 343 | SEQ ID NO: 8100 | 0.02% | 3.6 |

HLA A3 - 10 mers

| Maximum possible score using this molecule type | | | | 12150 |
|---|---|---|---|---|
| 1 | 255 | SEQ ID NO: 8101 | 1.48% | 180 |
| 2 | 180 | SEQ ID NO: 8102 | 0.55% | 67.5 |
| 3 | 768 | SEQ ID NO: 8103 | 0.49% | 60 |
| 4 | 1177 | SEQ ID NO: 8104 | 0.49% | 60 |
| 5 | 380 | SEQ ID NO: 8105 | 0.24% | 30 |
| 6 | 100 | SEQ ID NO: 8106 | 0.18% | 22.5 |
| 7 | 786 | SEQ ID NO: 8107 | 0.16% | 20 |
| 8 | 1217 | SEQ ID NO: 8108 | 0.16% | 20 |
| 9 | 207 | SEQ ID NO: 8109 | 0.14% | 18 |
| 10 | 1183 | SEQ ID NO: 8110 | 0.14% | 18 |
| 11 | 38 | SEQ ID NO: 8111 | 0.09% | 12 |
| 12 | 52 | SEQ ID NO: 8112 | 0.09% | 12 |
| 13 | 8 | SEQ ID NO: 8113 | 0.06% | 8 |
| 14 | 679 | SEQ ID NO: 8114 | 0.06% | 8 |
| 15 | 73 | SEQ ID NO: 8115 | 0.05% | 6.75 |
| 16 | 1204 | SEQ ID NO: 8116 | 0.05% | 6.075 |
| 17 | 50 | SEQ ID NO: 8117 | 0.04% | 6 |
| 18 | 774 | SEQ ID NO: 8118 | 0.04% | 6 |
| 19 | 845 | SEQ ID NO: 8119 | 0.04% | 6 |
| 20 | 214 | SEQ ID NO: 8120 | 0.04% | 5.4 |

HLA A24 - 9 mers

| Maximum possible score using this molecule type | | | | 1596.672 |
|---|---|---|---|---|
| 1 | 1118 | SEQ ID NO: 8121 | 19.84% | 316.8 |
| 2 | 51 | SEQ ID NO: 8122 | 18.78% | 300 |
| 3 | 161 | SEQ ID NO: 8123 | 18.78% | 300 |
| 4 | 434 | SEQ ID NO: 8124 | 18.78% | 300 |
| 5 | 365 | SEQ ID NO: 8125 | 13.77% | 220 |
| 6 | 736 | SEQ ID NO: 8126 | 12.52% | 200 |
| 7 | 620 | SEQ ID NO: 8127 | 7.51% | 120 |
| 8 | 1068 | SEQ ID NO: 8128 | 7.51% | 120 |
| 9 | 817 | SEQ ID NO: 8129 | 3.75% | 60 |
| 10 | 336 | SEQ ID NO: 8130 | 3.44% | 55 |
| 11 | 687 | SEQ ID NO: 8131 | 3.13% | 50 |
| 12 | 254 | SEQ ID NO: 8132 | 2.34% | 37.5 |
| 13 | 627 | SEQ ID NO: 8133 | 1.87% | 30 |
| 14 | 950 | SEQ ID NO: 8134 | 1.75% | 28 |
| 15 | 28 | SEQ ID NO: 8135 | 1.56% | 25 |
| 16 | 408 | SEQ ID NO: 8136 | 1.56% | 25 |
| 17 | 159 | SEQ ID NO: 8137 | 1.31% | 21 |
| 18 | 1166 | SEQ ID NO: 8138 | 1.26% | 20.16 |
| 19 | 45 | SEQ ID NO: 8139 | 1.25% | 20 |
| 20 | 185 | SEQ ID NO: 8140 | 1.25% | 20 |

HLA A24 - 10 mers

| Maximum possible score using this molecule type | | | | 1596.672 |
|---|---|---|---|---|
| 1 | 438 | SEQ ID NO: 8141 | 27.55% | 440 |
| 2 | 489 | SEQ ID NO: 8142 | 22.54% | 360 |
| 3 | 254 | SEQ ID NO: 8143 | 18.78% | 300 |
| 4 | 354 | SEQ ID NO: 8144 | 11.27% | 180 |
| 5 | 406 | SEQ ID NO: 8145 | 11.27% | 180 |
| 6 | 1047 | SEQ ID NO: 8146 | 11.27% | 180 |
| 7 | 473 | SEQ ID NO: 8147 | 7.51% | 120 |
| 8 | 350 | SEQ ID NO: 8148 | 6.26% | 100 |
| 9 | 769 | SEQ ID NO: 8149 | 6.26% | 100 |
| 10 | 193 | SEQ ID NO: 8150 | 5.63% | 90 |
| 11 | 479 | SEQ ID NO: 8151 | 3.13% | 50 |
| 12 | 0 | SEQ ID NO: 8152 | 2.70% | 43.2 |
| 13 | 813 | SEQ ID NO: 8153 | 1.87% | 30 |
| 14 | 739 | SEQ ID NO: 8154 | 1.50% | 24 |
| 15 | 782 | SEQ ID NO: 8155 | 1.50% | 24 |
| 16 | 1186 | SEQ ID NO: 8156 | 1.31% | 21 |
| 17 | 910 | SEQ ID NO: 8157 | 1.05% | 16.8 |
| 18 | 128 | SEQ ID NO: 8158 | 0.93% | 15 |
| 19 | 183 | SEQ ID NO: 8159 | 0.93% | 15 |
| 20 | 1069 | SEQ ID NO: 8160 | 0.93% | 15 |

HLA A 0201 - 9 mers

| Maximum possible score using this molecule type | | | | 3925227.1 |
|---|---|---|---|---|
| 1 | 1041 | SEQ ID NO: 8161 | 0.01% | 484.2379773 |
| 2 | 981 | SEQ ID NO: 8162 | 0.00% | 382.536 |
| 3 | 957 | SEQ ID NO: 8163 | 0.00% | 342.4606344 |
| 4 | 896 | SEQ ID NO: 8164 | 0.00% | 232.6931712 |
| 5 | 1173 | SEQ ID NO: 8165 | 0.00% | 201.447432 |
| 6 | 733 | SEQ ID NO: 8166 | 0.00% | 171.86796 |
| 7 | 410 | SEQ ID NO: 8167 | 0.00% | 135.45252 |
| 8 | 786 | SEQ ID NO: 8168 | 0.00% | 119.463012 |
| 9 | 150 | SEQ ID NO: 8169 | 0.00% | 102.17550222 |
| 10 | 1 | SEQ ID NO: 8170 | 0.00% | 94.98737754 |
| 11 | 595 | SEQ ID NO: 8171 | 0.00% | 93.239424 |
| 12 | 1095 | SEQ ID NO: 8172 | 0.00% | 89.41779 |
| 13 | 1166 | SEQ ID NO: 8173 | 0.00% | 87.58584 |
| 14 | 845 | SEQ ID NO: 8174 | 0.00% | 79.642008 |
| 15 | 734 | SEQ ID NO: 8175 | 0.00% | 73.47672 |
| 16 | 802 | SEQ ID NO: 8176 | 0.00% | 71.872056 |
| 17 | 1213 | SEQ ID NO: 8177 | 0.00% | 71.872056 |
| 18 | 105 | SEQ ID NO: 8178 | 0.00% | 50.232 |
| 19 | 939 | SEQ ID NO: 8179 | 0.00% | 49.13352 |
| 20 | 130 | SEQ ID NO: 8180 | 0.00% | 48.732354 |

HLA A 0201 - 10 mers

| Maximum possible score using this molecule type | | | | 3925227.1 |
|---|---|---|---|---|
| 1 | 372 | SEQ ID NO: 8181 | 0.04% | 1896.33528 |
| 2 | 410 | SEQ ID NO: 8182 | 0.02% | 1134.00849744 |
| 3 | 162 | SEQ ID NO: 8183 | 0.01% | 685.3897512 |
| 4 | 1076 | SEQ ID NO: 8184 | 0.01% | 640.90320525 |
| 5 | 1196 | SEQ ID NO: 8185 | 0.01% | 623.742666372 |
| 6 | 353 | SEQ ID NO: 8186 | 0.01% | 446.7384576 |
| 7 | 50 | SEQ ID NO: 8187 | 0.00% | 375.97824 |
| 8 | 733 | SEQ ID NO: 8188 | 0.00% | 271.863864 |
| 9 | 130 | SEQ ID NO: 8189 | 0.00% | 235.6873848 |
| 10 | 415 | SEQ ID NO: 8190 | 0.00% | 185.679 |
| 11 | 297 | SEQ ID NO: 8191 | 0.00% | 177.496704 |
| 12 | 1 | SEQ ID NO: 8192 | 0.00% | 152.42160582 |
| 13 | 56 | SEQ ID NO: 8193 | 0.00% | 110.013876 |
| 14 | 732 | SEQ ID NO: 8194 | 0.00% | 101.0988 |
| 15 | 6 | SEQ ID NO: 8195 | 0.00% | 98.26704 |
| 16 | 261 | SEQ ID NO: 8196 | 0.00% | 91.60164 |
| 17 | 1040 | SEQ ID NO: 8197 | 0.00% | 76.98537 |
| 18 | 928 | SEQ ID NO: 8198 | 0.00% | 71.2908 |
| 19 | 1188 | SEQ ID NO: 8199 | 0.00% | 69.81282 |
| 20 | 1094 | SEQ ID NO: 8200 | 0.00% | 52.5987 |

HLA A 1101 - 9 mers

| Maximum possible score using this molecule type | | | | 36 |
|---|---|---|---|---|
| 1 | 402 | SEQ ID NO: 8201 | 25% | 9 |
| 2 | 902 | SEQ ID NO: 8202 | 22.5% | 8.1 |
| 3 | 288 | SEQ ID NO: 8203 | 11.11% | 4 |
| 4 | 85 | SEQ ID NO: 8204 | 6.66% | 2.4 |
| 5 | 706 | SEQ ID NO: 8205 | 6.66% | 2.4 |
| 6 | 456 | SEQ ID NO: 8206 | 5.55% | 2 |
| 7 | 920 | SEQ ID NO: 8207 | 5.55% | 2 |
| 8 | 535 | SEQ ID NO: 8208 | 5% | 1.8 |
| 9 | 364 | SEQ ID NO: 8209 | 3.33% | 1.2 |
| 10 | 438 | SEQ ID NO: 8210 | 3.33% | 1.2 |
| 11 | 798 | SEQ ID NO: 8211 | 3.33% | 1.2 |
| 12 | 808 | SEQ ID NO: 8212 | 3.33% | 1.2 |
| 13 | 937 | SEQ ID NO: 8213 | 3.33% | 1.2 |
| 14 | 956 | SEQ ID NO: 8214 | 3.33% | 1.2 |
| 15 | 557 | SEQ ID NO: 8215 | 2.77% | 1 |
| 16 | 1218 | SEQ ID NO: 8216 | 2.77% | 1 |
| 17 | 784 | SEQ ID NO: 8217 | 2.5% | 0.9 |
| 18 | 249 | SEQ ID NO: 8218 | 2.22% | 0.8 |
| 19 | 768 | SEQ ID NO: 8219 | 2.22% | 0.8 |
| 20 | 1178 | SEQ ID NO: 8220 | 2.22% | 0.8 |

HLA A 1101 - 10 mers

| Maximum possible score using this molecule type | | | | 36 |
|---|---|---|---|---|
| 1 | 38 | SEQ ID NO: 8221 | 13.33% | 4.8 |
| 2 | 807 | SEQ ID NO: 8222 | 12.5% | 4.5 |
| 3 | 100 | SEQ ID NO: 8223 | 11.11% | 4 |
| 4 | 380 | SEQ ID NO: 8224 | 11.11% | 4 |
| 5 | 767 | SEQ ID NO: 8225 | 10% | 3.6 |
| 6 | 533 | SEQ ID NO: 8226 | 8.33% | 3 |
| 7 | 967 | SEQ ID NO: 8227 | 6.66% | 2.4 |
| 8 | 919 | SEQ ID NO: 8228 | 5.55% | 2 |
| 9 | 305 | SEQ ID NO: 8229 | 5% | 1.8 |
| 10 | 211 | SEQ ID NO: 8230 | 3.33% | 1.2 |
| 11 | 511 | SEQ ID NO: 8231 | 3.33% | 1.2 |
| 12 | 1177 | SEQ ID NO: 8232 | 3.33% | 1.2 |
| 13 | 429 | SEQ ID NO: 8233 | 2.77% | 1 |
| 14 | 758 | SEQ ID NO: 8234 | 2.77% | 1 |
| 15 | 797 | SEQ ID NO: 8235 | 2.5% | 0.9 |
| 16 | 255 | SEQ ID NO: 8236 | 2.22% | 0.8 |
| 17 | 986 | SEQ ID NO: 8237 | 2.22% | 0.8 |
| 18 | 1157 | SEQ ID NO: 8238 | 2.22% | 0.8 |
| 19 | 170 | SEQ ID NO: 8239 | 1.66% | 0.6 |
| 20 | 893 | SEQ ID NO: 8240 | 1.66% | 0.6 |

HLA B7 - 9 mers

| Maximum possible score using this molecule type | | | | 5400 |
|---|---|---|---|---|
| 1 | 200 | SEQ ID NO: 8241 | 1.48% | 80 |
| 2 | 1243 | SEQ ID NO: 8242 | 1.48% | 80 |
| 3 | 123 | SEQ ID NO: 8243 | 0.74% | 40 |
| 4 | 248 | SEQ ID NO: 8244 | 0.66% | 36 |
| 5 | 1036 | SEQ ID NO: 8245 | 0.66% | 36 |
| 6 | 494 | SEQ ID NO: 8246 | 0.37% | 20 |
| 7 | 495 | SEQ ID NO: 8247 | 0.37% | 20 |
| 8 | 523 | SEQ ID NO: 8248 | 0.37% | 20 |
| 9 | 842 | SEQ ID NO: 8249 | 0.37% | 20 |
| 10 | 932 | SEQ ID NO: 8250 | 0.37% | 20 |
| 11 | 274 | SEQ ID NO: 8251 | 0.33% | 18 |
| 12 | 588 | SEQ ID NO: 8252 | 0.22% | 12 |
| 13 | 656 | SEQ ID NO: 8253 | 0.22% | 12 |
| 14 | 657 | SEQ ID NO: 8254 | 0.22% | 12 |
| 15 | 767 | SEQ ID NO: 8255 | 0.22% | 12 |
| 16 | 911 | SEQ ID NO: 8256 | 0.22% | 12 |
| 17 | 939 | SEQ ID NO: 8257 | 0.22% | 12 |
| 18 | 1007 | SEQ ID NO: 8258 | 0.22% | 12 |
| 19 | 1170 | SEQ ID NO: 8259 | 0.22% | 12 |
| 20 | 1206 | SEQ ID NO: 8260 | 0.22% | 12 |

HLA B7 - 10 mers

| Maximum possible score using this molecule type | | | | 5400 |
|---|---|---|---|---|
| 1 | 505 | SEQ ID NO: 8261 | 4.44% | 240 |
| 2 | 312 | SEQ ID NO: 8262 | 3.70% | 200 |
| 3 | 141 | SEQ ID NO: 8263 | 1.11% | 60 |
| 4 | 1006 | SEQ ID NO: 8264 | 0.66% | 36 |
| 5 | 411 | SEQ ID NO: 8265 | 0.44% | 24 |
| 6 | 122 | SEQ ID NO: 8266 | 0.37% | 20 |
| 7 | 134 | SEQ ID NO: 8267 | 0.37% | 20 |
| 8 | 184 | SEQ ID NO: 8268 | 0.37% | 20 |
| 9 | 367 | SEQ ID NO: 8269 | 0.37% | 20 |
| 10 | 402 | SEQ ID NO: 8270 | 0.37% | 20 |
| 11 | 494 | SEQ ID NO: 8271 | 0.37% | 20 |
| 12 | 560 | SEQ ID NO: 8272 | 0.37% | 20 |
| 13 | 626 | SEQ ID NO: 8273 | 0.37% | 20 |
| 14 | 931 | SEQ ID NO: 8274 | 0.37% | 20 |
| 15 | 956 | SEQ ID NO: 8275 | 0.37% | 20 |
| 16 | 1117 | SEQ ID NO: 8276 | 0.37% | 20 |
| 17 | 1169 | SEQ ID NO: 8277 | 0.37% | 20 |
| 18 | 1196 | SEQ ID NO: 8278 | 0.37% | 20 |
| 19 | 247 | SEQ ID NO: 8279 | 0.22% | 12 |
| 20 | 273 | SEQ ID NO: 8280 | 0.22% | 12 |

TABLE 17
Epitopes for SEQ ID NO: 6043

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 168 | SEQ ID NO: 8281 | 0.2% | 11.25 |
| 2 | 212 | SEQ ID NO: 8282 | 0.08% | 4.5 |
| 3 | 223 | SEQ ID NO: 8283 | 0.08% | 4.5 |
| 4 | 104 | SEQ ID NO: 8284 | 0.04% | 2.5 |
| 5 | 170 | SEQ ID NO: 8285 | 0.04% | 2.5 |
| 6 | 99 | SEQ ID NO: 8286 | 0.04% | 2.25 |
| 7 | 188 | SEQ ID NO: 8287 | 0.02% | 1.35 |
| 8 | 180 | SEQ ID NO: 8288 | 0.02% | 1.25 |
| 9 | 219 | SEQ ID NO: 8289 | 0.02% | 1.25 |
| 10 | 18 | SEQ ID NO: 8290 | 0.01% | 1 |
| 11 | 226 | SEQ ID NO: 8291 | 0.01% | 1 |
| 12 | 98 | SEQ ID NO: 8292 | 0.01% | 0.625 |
| 13 | 151 | SEQ ID NO: 8293 | 0.01% | 0.625 |
| 14 | 10 | SEQ ID NO: 8294 | 0.01% | 0.6 |
| 15 | 13 | SEQ ID NO: 8295 | 0.00% | 0.5 |
| 16 | 32 | SEQ ID NO: 8296 | 0.00% | 0.5 |
| 17 | 70 | SEQ ID NO: 8297 | 0.00% | 0.5 |
| 18 | 78 | SEQ ID NO: 8298 | 0.00% | 0.5 |
| 19 | 82 | SEQ ID NO: 8299 | 0.00% | 0.5 |
| 20 | 145 | SEQ ID NO: 8300 | 0.00% | 0.5 |

HLA A1 - 10 mers

| Maximum possible score using this molecule type | | | | 5625 |
|---|---|---|---|---|
| 1 | 99 | SEQ ID NO: 8301 | 0.8% | 45 |
| 2 | 223 | SEQ ID NO: 8302 | 0.8% | 45 |
| 3 | 188 | SEQ ID NO: 8303 | 0.48% | 27 |
| 4 | 206 | SEQ ID NO: 8304 | 0.2% | 11.25 |
| 5 | 253 | SEQ ID NO: 8305 | 0.17% | 10 |
| 6 | 174 | SEQ ID NO: 8306 | 0.13% | 7.5 |
| 7 | 97 | SEQ ID NO: 8307 | 0.04% | 2.5 |
| 8 | 257 | SEQ ID NO: 8308 | 0.04% | 2.5 |
| 9 | 179 | SEQ ID NO: 8309 | 0.04% | 2.25 |
| 10 | 162 | SEQ ID NO: 8310 | 0.02% | 1.25 |
| 11 | 196 | SEQ ID NO: 8311 | 0.02% | 1.25 |
| 12 | 219 | SEQ ID NO: 8312 | 0.02% | 1.25 |
| 13 | 18 | SEQ ID NO: 8313 | 0.01% | 1 |
| 14 | 246 | SEQ ID NO: 8314 | 0.01% | 1 |
| 15 | 38 | SEQ ID NO: 8315 | 0.01% | 0.75 |
| 16 | 33 | SEQ ID NO: 8316 | 0.00% | 0.5 |
| 17 | 69 | SEQ ID NO: 8317 | 0.00% | 0.5 |
| 18 | 81 | SEQ ID NO: 8318 | 0.00% | 0.5 |
| 19 | 104 | SEQ ID NO: 8319 | 0.00% | 0.5 |
| 20 | 116 | SEQ ID NO: 8320 | 0.00% | 0.5 |

HLA A3 - 9 mers

| Maximum possible score using this molecule type | | | | 12150 |
|---|---|---|---|---|
| 1 | 104 | SEQ ID NO: 8321 | 0.98% | 120 |
| 2 | 123 | SEQ ID NO: 8322 | 0.74% | 90 |
| 3 | 82 | SEQ ID NO: 8323 | 0.44% | 54 |
| 4 | 106 | SEQ ID NO: 8324 | 0.11% | 13.5 |
| 5 | 99 | SEQ ID NO: 8325 | 0.08% | 10.8 |
| 6 | 127 | SEQ ID NO: 8326 | 0.08% | 10 |
| 7 | 71 | SEQ ID NO: 8327 | 0.07% | 9 |
| 8 | 1 | SEQ ID NO: 8328 | 0.06% | 8.1 |
| 9 | 113 | SEQ ID NO: 8329 | 0.04% | 6 |
| 10 | 84 | SEQ ID NO: 8330 | 0.03% | 4.5 |
| 11 | 109 | SEQ ID NO: 8331 | 0.03% | 4.05 |
| 12 | 58 | SEQ ID NO: 8332 | 0.02% | 3 |
| 13 | 138 | SEQ ID NO: 8333 | 0.02% | 3 |
| 14 | 44 | SEQ ID NO: 8334 | 0.02% | 2.7 |
| 15 | 81 | SEQ ID NO: 8335 | 0.02% | 2.7 |
| 16 | 226 | SEQ ID NO: 8336 | 0.02% | 2.7 |
| 17 | 184 | SEQ ID NO: 8337 | 0.01% | 1.8 |
| 18 | 102 | SEQ ID NO: 8338 | 0.01% | 1.215 |
| 19 | 39 | SEQ ID NO: 8339 | 0.00% | 1.2 |
| 20 | 234 | SEQ ID NO: 8340 | 0.00% | 0.9 |

HLA A3 - 10 mers

| Maximum possible score using this molecule type | | | | 12150 |
|---|---|---|---|---|
| 1 | 99 | SEQ ID NO: 8341 | 1.33% | 162 |
| 2 | 81 | SEQ ID NO: 8342 | 0.44% | 54 |
| 3 | 104 | SEQ ID NO: 8343 | 0.24% | 30 |
| 4 | 51 | SEQ ID NO: 8344 | 0.16% | 20 |
| 5 | 122 | SEQ ID NO: 8345 | 0.11% | 13.5 |
| 6 | 71 | SEQ ID NO: 8346 | 0.07% | 9 |
| 7 | 69 | SEQ ID NO: 8347 | 0.04% | 6 |
| 8 | 223 | SEQ ID NO: 8348 | 0.04% | 5.4 |
| 9 | 84 | SEQ ID NO: 8349 | 0.03% | 4.5 |
| 10 | 63 | SEQ ID NO: 8350 | 0.02% | 3.6 |
| 11 | 138 | SEQ ID NO: 8351 | 0.02% | 3 |
| 12 | 201 | SEQ ID NO: 8352 | 0.01% | 1.8 |
| 13 | 44 | SEQ ID NO: 8353 | 0.01% | 1.35 |
| 14 | 83 | SEQ ID NO: 8354 | 0.01% | 1.35 |
| 15 | 116 | SEQ ID NO: 8355 | 0.00% | 1.2 |
| 16 | 46 | SEQ ID NO: 8356 | 0.00% | 0.9 |
| 17 | 183 | SEQ ID NO: 8357 | 0.00% | 0.81 |
| 18 | 57 | SEQ ID NO: 8358 | 0.00% | 0.6 |
| 19 | 93 | SEQ ID NO: 8359 | 0.00% | 0.6 |
| 20 | 113 | SEQ ID NO: 8360 | 0.00% | 0.6 |

HLA A24 - 9 mers

| Maximum possible score using this molecule type | | | | 1596.672 |
|---|---|---|---|---|
| 1 | 198 | SEQ ID NO: 8361 | 13.15% | 210 |
| 2 | 105 | SEQ ID NO: 8362 | 9.39% | 150 |
| 3 | 210 | SEQ ID NO: 8363 | 4.69% | 75 |
| 4 | 75 | SEQ ID NO: 8364 | 3.15% | 50.4 |
| 5 | 85 | SEQ ID NO: 8365 | 2.63% | 42 |
| 6 | 205 | SEQ ID NO: 8366 | 2.10% | 33.6 |
| 7 | 77 | SEQ ID NO: 8367 | 1.87% | 30 |
| 8 | 158 | SEQ ID NO: 8368 | 0.65% | 10.5 |
| 9 | 103 | SEQ ID NO: 8369 | 0.56% | 9 |
| 10 | 227 | SEQ ID NO: 8370 | 0.55% | 8.8704 |
| 11 | 32 | SEQ ID NO: 8371 | 0.54% | 8.64 |
| 12 | 74 | SEQ ID NO: 8372 | 0.50% | 8 |
| 13 | 131 | SEQ ID NO: 8373 | 0.50% | 8 |
| 14 | 54 | SEQ ID NO: 8374 | 0.46% | 7.5 |
| 15 | 99 | SEQ ID NO: 8375 | 0.45% | 7.2 |
| 16 | 44 | SEQ ID NO: 8376 | 0.37% | 6 |
| 17 | 62 | SEQ ID NO: 8377 | 0.37% | 6 |
| 18 | 87 | SEQ ID NO: 8378 | 0.37% | 6 |
| 19 | 89 | SEQ ID NO: 8379 | 0.37% | 6 |
| 20 | 154 | SEQ ID NO: 8380 | 0.37% | 6 |

HLA A24 - 10 mers

| Maximum possible score using this molecule type | | | | 1596.672 |
|---|---|---|---|---|
| 1 | 105 | SEQ ID NO: 8381 | 22.54% | 360 |
| 2 | 204 | SEQ ID NO: 8382 | 17.53% | 280 |
| 3 | 209 | SEQ ID NO: 8383 | 3.13% | 50 |
| 4 | 75 | SEQ ID NO: 8384 | 1.87% | 30 |
| 5 | 85 | SEQ ID NO: 8385 | 1.87% | 30 |
| 6 | 77 | SEQ ID NO: 8386 | 1.12% | 18 |
| 7 | 74 | SEQ ID NO: 8387 | 0.84% | 13.44 |
| 8 | 210 | SEQ ID NO: 8388 | 0.56% | 9 |
| 9 | 226 | SEQ ID NO: 8389 | 0.55% | 8.8704 |
| 10 | 98 | SEQ ID NO: 8390 | 0.54% | 8.64 |
| 11 | 198 | SEQ ID NO: 8391 | 0.46% | 7.5 |
| 12 | 67 | SEQ ID NO: 8392 | 0.45% | 7.2 |
| 13 | 152 | SEQ ID NO: 8393 | 0.43% | 7 |
| 14 | 43 | SEQ ID NO: 8394 | 0.37% | 6 |
| 15 | 63 | SEQ ID NO: 8395 | 0.37% | 6 |
| 16 | 72 | SEQ ID NO: 8396 | 0.37% | 6 |
| 17 | 89 | SEQ ID NO: 8397 | 0.37% | 6 |
| 18 | 101 | SEQ ID NO: 8398 | 0.37% | 6 |
| 19 | 107 | SEQ ID NO: 8399 | 0.37% | 6 |
| 20 | 111 | SEQ ID NO: 8400 | 0.37% | 6 |

HLA A 0201 - 9 mers

| Maximum possible score using this molecule type | | | | 3925227.1 |
|---|---|---|---|---|
| 1 | 138 | SEQ ID NO: 8401 | 0.21% | 8532.082944 |
| 2 | 106 | SEQ ID NO: 8402 | 0.10% | 3977.8497792 |
| 3 | 44 | SEQ ID NO: 8403 | 0.03% | 1243.078056 |
| 4 | 71 | SEQ ID NO: 8404 | 0.00% | 348.872832 |
| 5 | 234 | SEQ ID NO: 8405 | 0.00% | 243.432 |
| 6 | 51 | SEQ ID NO: 8406 | 0.00% | 130.26096 |
| 7 | 109 | SEQ ID NO: 8407 | 0.00% | 91.182672 |
| 8 | 81 | SEQ ID NO: 8408 | 0.00% | 73.342584 |
| 9 | 88 | SEQ ID NO: 8409 | 0.00% | 70.386624 |
| 10 | 1 | SEQ ID NO: 8410 | 0.00% | 65.32728732 |
| 11 | 38 | SEQ ID NO: 8411 | 0.00% | 47.876409 |
| 12 | 76 | SEQ ID NO: 8412 | 0.00% | 36.8637882 |
| 13 | 46 | SEQ ID NO: 8413 | 0.00% | 30.889782 |
| 14 | 211 | SEQ ID NO: 8414 | 0.00% | 21.616753941 |
| 15 | 201 | SEQ ID NO: 8415 | 0.00% | 19.657134 |
| 16 | 102 | SEQ ID NO: 8416 | 0.00% | 18.4318941 |
| 17 | 199 | SEQ ID NO: 8417 | 0.00% | 16.496865 |
| 18 | 74 | SEQ ID NO: 8418 | 0.00% | 15.783256167 |
| 19 | 62 | SEQ ID NO: 8419 | 0.00% | 13.9968225 |
| 20 | 99 | SEQ ID NO: 8420 | 0.00% | 10.31851392 |

HLA A 0201 - 10 mers

| Maximum possible score using this molecule type | | | | 3925227.1 |
|---|---|---|---|---|
| 1 | 78 | SEQ ID NO: 8421 | 0.01% | 556.494246 |
| 2 | 138 | SEQ ID NO: 8422 | 0.01% | 395.245972224 |
| 3 | 84 | SEQ ID NO: 8423 | 0.00% | 201.554244 |
| 4 | 71 | SEQ ID NO: 8424 | 0.00% | 143.65707264 |
| 5 | 44 | SEQ ID NO: 8425 | 0.00% | 132.54624 |
| 6 | 76 | SEQ ID NO: 8426 | 0.00% | 84.78671286 |
| 7 | 8 | SEQ ID NO: 8427 | 0.00% | 69.552 |
| 8 | 211 | SEQ ID NO: 8428 | 0.00% | 52.7237901 |
| 9 | 113 | SEQ ID NO: 8429 | 0.00% | 47.99088 |
| 10 | 61 | SEQ ID NO: 8430 | 0.00% | 37.4509575 |
| 11 | 93 | SEQ ID No: 8431 | 0.00% | 31.24872 |
| 12 | 137 | SEQ ID NO: 8432 | 0.00% | 31.1384304 |
| 13 | 37 | SEQ ID NO: 8433 | 0.00% | 27.531 |
| 14 | 55 | SEQ ID NO: 8434 | 0.00% | 22.9153278 |
| 15 | 98 | SEQ ID NO: 8435 | 0.00% | 22.1063618985 |
| 16 | 108 | SEQ ID NO: 8436 | 0.00% | 21.55457052 |
| 17 | 63 | SEQ ID NO: 8437 | 0.00% | 21.3624 |
| 18 | 45 | SEQ ID NO: 8438 | 0.00% | 19.657134 |
| 19 | 200 | SEQ ID NO: 8439 | 0.00% | 19.657134 |
| 20 | 104 | SEQ ID NO: 8440 | 0.00% | 13.87622016 |

HLA A 1101 - 9 mers

| Maximum possible score using this molecule type | | | | 36 |
|---|---|---|---|---|
| 1 | 58 | SEQ ID NO: 8441 | 5.55% | 2 |
| 2 | 125 | SEQ ID NO: 8442 | 1.66% | 0.6 |
| 3 | 226 | SEQ ID NO: 8443 | 1.66% | 0.6 |
| 4 | 229 | SEQ ID NO: 8444 | 1.66% | 0.6 |

HLA A 1101 - 10 mers

| Maximum possible score using this molecule type | | | | 36 |
|---|---|---|---|---|
| 1 | 122 | SEQ ID NO: 8445 | 2.22% | 0.8 |
| 2 | 228 | SEQ ID NO: 8446 | 2.22% | 0.8 |

HLA B7 - 9 mers

| Maximum possible score using this molecule type | | | | 5400 |
|---|---|---|---|---|
| 1 | 97 | SEQ ID NO: 8447 | 0.66% | 36 |
| 2 | 86 | SEQ ID NO: 8448 | 0.37% | 20 |
| 3 | 37 | SEQ ID NO: 8449 | 0.33% | 18 |
| 4 | 62 | SEQ ID NO: 8450 | 0.33% | 18 |
| 5 | 32 | SEQ ID NO: 8451 | 0.22% | 12 |
| 6 | 102 | SEQ ID NO: 8452 | 0.22% | 12 |
| 7 | 227 | SEQ ID NO: 8453 | 0.22% | 12 |
| 8 | 53 | SEQ ID NO: 8454 | 0.11% | 6 |
| 9 | 1 | SEQ ID NO: 8455 | 0.07% | 4 |
| 10 | 44 | SEQ ID NO: 8456 | 0.07% | 4 |
| 11 | 56 | SEQ ID NO: 8457 | 0.07% | 4 |
| 12 | 64 | SEQ ID NO: 8458 | 0.07% | 4 |
| 13 | 74 | SEQ ID NO: 8459 | 0.07% | 4 |
| 14 | 76 | SEQ ID NO: 8460 | 0.07% | 4 |
| 15 | 87 | SEQ ID NO: 8461 | 0.07% | 4 |
| 16 | 106 | SEQ ID NO: 8462 | 0.07% | 4 |
| 17 | 131 | SEQ ID NO: 8463 | 0.07% | 4 |
| 18 | 23 | SEQ ID NO: 8464 | 0.03% | 2 |
| 19 | 157 | SEQ ID NO: 8465 | 0.03% | 2 |
| 20 | 166 | SEQ ID NO: 8466 | 0.03% | 2 |

HLA B7 - 10 mers

| Maximum possible score using this molecule type | | | | 5400 |
|---|---|---|---|---|
| 1 | 119 | SEQ ID NO: 8467 | 3.33% | 180 |
| 2 | 264 | SEQ ID NO: 8468 | 1.48% | 80 |
| 3 | 98 | SEQ ID NO: 8469 | 0.66% | 36 |
| 4 | 27 | SEQ ID NO: 8470 | 0.37% | 20 |
| 5 | 86 | SEQ ID NO: 8471 | 0.37% | 20 |
| 6 | 31 | SEQ ID NO: 8472 | 0.22% | 12 |
| 7 | 63 | SEQ ID NO: 8473 | 0.22% | 12 |
| 8 | 96 | SEQ ID NO: 8474 | 0.22% | 12 |
| 9 | 101 | SEQ ID NO: 8475 | 0.22% | 12 |
| 10 | 226 | SEQ ID NO: 8476 | 0.22% | 12 |
| 11 | 157 | SEQ ID NO: 8477 | 0.14% | 8 |
| 12 | 176 | SEQ ID NO: 8478 | 0.14% | 8 |
| 13 | 238 | SEQ ID NO: 8479 | 0.14% | 8 |
| 14 | 36 | SEQ ID NO: 8480 | 0.11% | 6 |
| 15 | 53 | SEQ ID NO: 8481 | 0.11% | 6 |
| 16 | 61 | SEQ ID NO: 8482 | 0.11% | 6 |
| 17 | 3 | SEQ ID NO: 8483 | 0.07% | 4 |
| 18 | 40 | SEQ ID NO: 8484 | 0.07% | 4 |
| 19 | 55 | SEQ ID NO: 8485 | 0.07% | 4 |
| 20 | 74 | SEQ ID NO: 8486 | 0.07% | 4 |

TABLE 18
Epitopes for SEQ ID NO: 6044

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 69 | SEQ ID NO: 8487 | 0.04% | 2.5 |
| 2 | 89 | SEQ ID NO: 8488 | 0.02% | 1.5 |
| 3 | 141 | SEQ ID NO: 8489 | 0.01% | 1 |
| 4 | 113 | SEQ ID NO: 8490 | 0.00% | 0.5 |

HLA A1 - 10 mers

| Maximum possible score using this molecule type | | | | 5625 |
|---|---|---|---|---|
| 1 | 21 | SEQ ID NO: 8491 | 0.02% | 1.5 |
| 2 | 88 | SEQ ID NO: 8492 | 0.02% | 1.5 |
| 3 | 8 | SEQ ID NO: 8493 | 0.02% | 1.25 |
| 4 | 31 | SEQ ID NO: 8494 | 0.00% | 0.5 |
| 5 | 112 | SEQ ID NO: 8495 | 0.00% | 0.5 |

HLA A3 - 9 mers

| Maximum possible score using this molecule type | | | | 12150 |
|---|---|---|---|---|
| 1 | 60 | SEQ ID NO: 8496 | 1.23% | 150 |
| 2 | 77 | SEQ ID NO: 8497 | 1.11% | 135 |
| 3 | 141 | SEQ ID NO: 8498 | 0.49% | 60 |
| 4 | 95 | SEQ ID NO: 8499 | 0.32% | 40 |
| 5 | 128 | SEQ ID NO: 8500 | 0.08% | 10 |
| 6 | 113 | SEQ ID NO: 8501 | 0.04% | 6 |
| 7 | 69 | SEQ ID NO: 8502 | 0.01% | 2 |
| 8 | 22 | SEQ ID NO: 8503 | 0.01% | 1.8 |
| 9 | 42 | SEQ ID NO: 8504 | 0.01% | 1.8 |
| 10 | 78 | SEQ ID NO: 8505 | 0.00% | 1.2 |
| 11 | 32 | SEQ ID NO: 8506 | 0.00% | 1 |
| 12 | 54 | SEQ ID NO: 8507 | 0.00% | 0.9 |
| 13 | 74 | SEQ ID NO: 8508 | 0.00% | 0.9 |
| 14 | 28 | SEQ ID NO: 8509 | 0.00% | 0.6 |
| 15 | 36 | SEQ ID NO: 8510 | 0.00% | 0.6 |
| 16 | 48 | SEQ ID NO: 8511 | 0.00% | 0.6 |
| 17 | 118 | SEQ ID NO: 8512 | 0.00% | 0.6 |
| 18 | 4 | SEQ ID NO: 8513 | 0.00% | 0.5 |

HLA A3 - 10 mers

| Maximum possible score using this molecule type | | | | 12150 |
|---|---|---|---|---|
| 1 | 94 | SEQ ID NO: 8514 | 0.49% | 60 |
| 2 | 48 | SEQ ID NO: 8515 | 0.16% | 20 |
| 3 | 128 | SEQ ID NO: 8516 | 0.16% | 20 |
| 4 | 60 | SEQ ID NO: 8517 | 0.12% | 15 |
| 5 | 127 | SEQ ID NO: 8518 | 0.12% | 15 |
| 6 | 25 | SEQ ID NO: 8519 | 0.04% | 6 |
| 7 | 95 | SEQ ID NO: 8520 | 0.04% | 6 |
| 8 | 141 | SEQ ID NO: 8521 | 0.04% | 6 |
| 9 | 41 | SEQ ID NO: 8522 | 0.04% | 5.4 |
| 10 | 77 | SEQ ID NO: 8523 | 0.04% | 5.4 |
| 11 | 116 | SEQ ID NO: 8524 | 0.04% | 5.4 |
| 12 | 91 | SEQ ID NO: 8525 | 0.03% | 4 |
| 13 | 4 | SEQ ID NO: 8526 | 0.01% | 2 |
| 14 | 112 | SEQ ID NO: 8527 | 0.01% | 1.8 |
| 15 | 113 | SEQ ID NO: 8528 | 0.01% | 1.35 |
| 16 | 12 | SEQ ID NO: 8529 | 0.00% | 1.2 |
| 17 | 31 | SEQ ID NO: 8530 | 0.00% | 1 |
| 18 | 32 | SEQ ID NO: 8531 | 0.00% | 1 |
| 19 | 15 | SEQ ID NO: 8532 | 0.00% | 0.9 |
| 20 | 27 | SEQ ID NO: 8533 | 0.00% | 0.9 |

HLA A24 - 9 mers

| Maximum possible score using this molecule type | | | | 1596.672 |
|---|---|---|---|---|
| 1 | 61 | SEQ ID NO: 8534 | 14.46% | 231 |
| 2 | 16 | SEQ ID NO: 8535 | 3.13% | 50 |
| 3 | 120 | SEQ ID NO: 8536 | 1.87% | 30 |
| 4 | 41 | SEQ ID NO: 8537 | 0.60% | 9.6 |
| 5 | 71 | SEQ ID NO: 8538 | 0.45% | 7.2 |
| 6 | 21 | SEQ ID NO: 8539 | 0.37% | 6 |
| 7 | 53 | SEQ ID NO: 8540 | 0.37% | 6 |
| 8 | 65 | SEQ ID NO: 8541 | 0.37% | 6 |
| 9 | 121 | SEQ ID NO: 8542 | 0.37% | 6 |
| 10 | 74 | SEQ ID NO: 8543 | 0.36% | 5.76 |
| 11 | 20 | SEQ ID NO: 8544 | 0.35% | 5.6 |
| 12 | 79 | SEQ ID NO: 8545 | 0.35% | 5.6 |
| 13 | 105 | SEQ ID NO: 8546 | 0.33% | 5.28 |
| 14 | 48 | SEQ ID NO: 8547 | 0.30% | 4.8 |
| 15 | 88 | SEQ ID NO: 8548 | 0.30% | 4.8 |
| 16 | 106 | SEQ ID NO: 8549 | 0.30% | 4.8 |
| 17 | 37 | SEQ ID NO: 8550 | 0.27% | 4.4 |
| 18 | 70 | SEQ ID NO: 8551 | 0.27% | 4.4 |
| 19 | 18 | SEQ ID NO: 8552 | 0.25% | 4 |
| 20 | 57 | SEQ ID NO: 8553 | 0.22% | 3.6 |

HLA A24 - 10 mers

| Maximum possible score using this molecule type | | | | 1596.672 |
|---|---|---|---|---|
| 1 | 120 | SEQ ID NO: 8554 | 1.87% | 30 |
| 2 | 73 | SEQ ID NO: 8555 | 0.54% | 8.64 |
| 3 | 19 | SEQ ID NO: 8556 | 0.52% | 8.4 |
| 4 | 78 | SEQ ID NO: 8557 | 0.52% | 8.4 |
| 5 | 104 | SEQ ID NO: 8558 | 0.49% | 7.92 |
| 6 | 61 | SEQ ID NO: 8559 | 0.46% | 7.5 |
| 7 | 47 | SEQ ID NO: 8560 | 0.45% | 7.2 |
| 8 | 36 | SEQ ID NO: 8561 | 0.41% | 6.6 |
| 9 | 52 | SEQ ID NO: 8562 | 0.37% | 6 |
| 10 | 64 | SEQ ID NO: 8563 | 0.30% | 4.8 |
| 11 | 70 | SEQ ID NO: 8564 | 0.30% | 4.8 |
| 12 | 105 | SEQ ID NO: 8565 | 0.30% | 4.8 |
| 13 | 123 | SEQ ID NO: 8566 | 0.30% | 4.8 |
| 14 | 69 | SEQ ID NO: 8567 | 0.27% | 4.4 |
| 15 | 20 | SEQ ID NO: 8568 | 0.25% | 4 |
| 16 | 66 | SEQ ID NO: 8569 | 0.25% | 4 |
| 17 | 83 | SEQ ID NO: 8570 | 0.25% | 4 |
| 18 | 86 | SEQ ID NO: 8571 | 0.25% | 4 |
| 19 | 101 | SEQ ID NO: 8572 | 0.25% | 4 |
| 20 | 119 | SEQ ID NO: 8573 | 0.25% | 4 |

HLA A 0201 - 9 mers

| Maximum possible score using this molecule type | | | | 3925227.1 |
|---|---|---|---|---|
| 1 | 62 | SEQ ID NO: 8574 | 0.00% | 136.1646 |
| 2 | 85 | SEQ ID NO: 8575 | 0.00% | 69.6969 |
| 3 | 47 | SEQ ID NO: 8576 | 0.00% | 60.153786 |
| 4 | 121 | SEQ ID NO: 8577 | 0.00% | 52.5182736 |
| 5 | 74 | SEQ ID NO: 8578 | 0.00% | 49.13352 |
| 6 | 23 | SEQ ID NO: 8579 | 0.00% | 21.99582 |
| 7 | 78 | SEQ ID NO: 8580 | 0.00% | 19.42488 |
| 8 | 114 | SEQ ID NO: 8581 | 0.00% | 14.6900655 |
| 9 | 4 | SEQ ID NO: 8582 | 0.00% | 11.304684 |
| 10 | 79 | SEQ ID NO: 8583 | 0.00% | 8.4687081 |
| 11 | 122 | SEQ ID NO: 8584 | 0.00% | 6.0996 |
| 12 | 100 | SEQ ID NO: 8585 | 0.00% | 5.382 |
| 13 | 105 | SEQ ID NO: 8586 | 0.00% | 4.981593 |
| 14 | 25 | SEQ ID NO: 8587 | 0.00% | 4.968 |
| 15 | 115 | SEQ ID NO: 8588 | 0.00% | 4.966482 |
| 16 | 24 | SEQ ID NO: 8589 | 0.00% | 4.4815221585 |
| 17 | 111 | SEQ ID NO: 8590 | 0.00% | 4.128201 |
| 18 | 94 | SEQ ID NO: 8591 | 0.00% | 3.67632 |
| 19 | 34 | SEQ ID NO: 8592 | 0.00% | 3.47553 |
| 20 | 12 | SEQ ID NO: 8593 | 0.00% | 3.30993 |

HLA A 0201 - 10 mers

| Maximum possible score using this molecule type | | | | 3925227.1 |
|---|---|---|---|---|
| 1 | 77 | SEQ ID NO: 8594 | 0.00% | 147.97188 |
| 2 | 62 | SEQ ID NO: 8595 | 0.00% | 143.59176 |
| 3 | 113 | SEQ ID NO: 8596 | 0.00% | 106.83684 |
| 4 | 78 | SEQ ID NO: 8597 | 0.00% | 83.526984 |
| 5 | 86 | SEQ ID NO: 8598 | 0.00% | 83.526984 |
| 6 | 74 | SEQ ID NO: 8599 | 0.00% | 69.552 |
| 7 | 121 | SEQ ID NO: 8600 | 0.00% | 61.06776 |
| 8 | 12 | SEQ ID NO: 8601 | 0.00% | 50.232 |
| 9 | 44 | SEQ ID NO: 8602 | 0.00% | 26.082 |
| 10 | 4 | SEQ ID NO: 8603 | 0.00% | 18.3816 |
| 11 | 0 | SEQ ID NO: 8604 | 0.00% | 17.38386 |
| 12 | 72 | SEQ ID NO: 8605 | 0.00% | 17.1396 |
| 13 | 22 | SEQ ID NO: 8606 | 0.00% | 16.21914 |
| 14 | 122 | SEQ ID NO: 8607 | 0.00% | 14.02908 |
| 15 | 64 | SEQ ID NO: 8608 | 0.00% | 11.161854 |
| 16 | 46 | SEQ ID NO: 8609 | 0.00% | 10.34586 |
| 17 | 54 | SEQ ID NO: 8610 | 0.00% | 8.846145 |
| 18 | 47 | SEQ ID NO: 8611 | 0.00% | 7.575080337 |
| 19 | 131 | SEQ ID NO: 8612 | 0.00% | 7.452 |
| 20 | 114 | SEQ ID NO: 8613 | 0.00% | 6.735366 |

HLA A 1101 - 9 mers

| Maximum possible score using this molecule type | | | | 36 |
|---|---|---|---|---|
| 1 | 69 | SEQ ID NO: 8614 | 5.55% | 2 |
| 2 | 22 | SEQ ID NO: 8615 | 5% | 1.8 |
| 3 | 77 | SEQ ID NO: 8616 | 5% | 1.8 |
| 4 | 141 | SEQ ID NO: 8617 | 3.33% | 1.2 |
| 5 | 60 | SEQ ID NO: 8618 | 2.22% | 0.8 |
| 6 | 95 | SEQ ID NO: 8619 | 2.22% | 0.8 |
| 7 | 36 | SEQ ID NO: 8620 | 1.66% | 0.6 |

HLA A 1101 - 10 mers

| Maximum possible score using this molecule type | | | | 36 |
|---|---|---|---|---|
| 1 | 41 | SEQ ID NO: 8621 | 3.33% | 1.2 |
| 2 | 68 | SEQ ID NO: 8622 | 33.33% | 1.2 |
| 3 | 94 | SEQ ID NO: 8623 | 3.33% | 1.2 |
| 4 | 31 | SEQ ID NO: 8624 | 2.77% | 1 |
| 5 | 127 | SEQ ID NO: 8625 | 2.5% | 0.9 |

HLA B7 - 9 mers

| Maximum possible score using this molecule type | | | | 5400 |
|---|---|---|---|---|
| 1 | 48 | SEQ ID NO: 8626 | 0.74% | 40 |
| 2 | 20 | SEQ ID NO: 8627 | 0.37% | 20 |
| 3 | 121 | SEQ ID NO: 8628 | 0.33% | 18 |
| 4 | 18 | SEQ ID NO: 8629 | 0.07% | 4 |
| 5 | 21 | SEQ ID NO: 8630 | 0.07% | 4 |
| 6 | 37 | SEQ ID NO: 8631 | 0.07% | 4 |
| 7 | 41 | SEQ ID NO: 8632 | 0.07% | 4 |
| 8 | 53 | SEQ ID NO: 8633 | 0.07% | 4 |
| 9 | 65 | SEQ ID NO: 8634 | 0.07% | 4 |
| 10 | 70 | SEQ ID NO: 8635 | 0.07% | 4 |
| 11 | 71 | SEQ ID NO: 8636 | 0.07% | 4 |
| 12 | 74 | SEQ ID NO: 8637 | 0.07% | 4 |
| 13 | 79 | SEQ ID NO: 8638 | 0.07% | 4 |
| 14 | 88 | SEQ ID NO: 8639 | 0.07% | 4 |
| 15 | 105 | SEQ ID NO: 8640 | 0.07% | 4 |
| 16 | 106 | SEQ ID NO: 8641 | 0.07% | 4 |
| 17 | 124 | SEQ ID NO: 8642 | 0.07% | 4 |
| 18 | 1 | SEQ ID NO: 8643 | 0.03% | 2 |
| 19 | 120 | SEQ ID NO: 8644 | 0.03% | 1.8 |
| 20 | 11 | SEQ ID NO: 8645 | 0.02% | 1.2 |

HLA B7 - 10 mers

| Maximum possible score using this molecule type | | | | 5400 |
|---|---|---|---|---|
| 1 | 63 | SEQ ID NO: 8646 | 1.48% | 80 |
| 2 | 123 | SEQ ID NO: 8647 | 0.74% | 40 |
| 3 | 20 | SEQ ID NO: 8648 | 0.37% | 20 |
| 4 | 64 | SEQ ID NO: 8649 | 0.22% | 12 |
| 5 | 119 | SEQ ID NO: 8650 | 0.11% | 6 |
| 6 | 54 | SEQ ID NO: 8651 | 0.09% | 5 |
| 7 | 19 | SEQ ID NO: 8652 | 0.07% | 4 |
| 8 | 36 | SEQ ID NO: 8653 | 0.07% | 4 |
| 9 | 47 | SEQ ID NO: 8654 | 0.07% | 4 |
| 10 | 52 | SEQ ID NO: 8655 | 0.07% | 4 |
| 11 | 69 | SEQ ID NO: 8656 | 0.07% | 4 |
| 12 | 70 | SEQ ID NO: 8657 | 0.07% | 4 |
| 13 | 73 | SEQ ID NO: 8658 | 0.07% | 4 |
| 14 | 78 | SEQ ID NO: 8659 | 0.07% | 4 |
| 15 | 83 | SEQ ID NO: 8660 | 0.07% | 4 |
| 16 | 86 | SEQ ID NO: 8661 | 0.07% | 4 |
| 17 | 101 | SEQ ID NO: 8662 | 0.07% | 4 |
| 18 | 104 | SEQ ID NO: 8663 | 0.07% | 4 |
| 19 | 105 | SEQ ID NO: 8664 | 0.07% | 4 |
| 20 | 15 | SEQ ID NO: 8665 | 0.03% | 2 |

TABLE 19
Epitopes for SEQ ID NO: 6045

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 4 | SEQ ID NO: 8666 | 0.02% | 1.35 |
| 2 | 66 | SEQ ID NO: 8667 | 0.02% | 1.35 |
| 3 | 33 | SEQ ID NO: 8668 | 0.02% | 1.25 |
| 4 | 44 | SEQ ID NO: 8669 | 0.01% | 1 |
| 5 | 50 | SEQ ID NO: 8670 | 0.01% | 1 |
| 6 | 14 | SEQ ID NO: 8671 | 0.01% | 0.75 |
| 7 | 48 | SEQ ID NO: 8672 | 0.01% | 0.75 |
| 8 | 11 | SEQ ID NO: 8673 | 0.00% | 0.5 |

HLA A1 - 10 mers

| Maximum possible score using this molecule type | | | | 5625 |
|---|---|---|---|---|
| 1 | 4 | SEQ ID NO: 8674 | 0.12% | 6.75 |
| 2 | 66 | SEQ ID NO: 8675 | 0.12% | 6.75 |
| 3 | 10 | SEQ ID NO: 8676 | 0.00% | 0.5 |
| 4 | 28 | SEQ ID NO: 8677 | 0.00% | 0.5 |
| 5 | 32 | SEQ ID NO: 8678 | 0.00% | 0.5 |
| 6 | 47 | SEQ ID NO: 8679 | 0.00% | 0.5 |

HLA A3 - 9 mers

| Maximum possible score using this molecule type | | | | 12150 |
|---|---|---|---|---|
| 1 | 17 | SEQ ID NO: 8680 | 0.24% | 30 |
| 2 | 44 | SEQ ID NO: 8681 | 0.07% | 9 |
| 3 | 19 | SEQ ID NO: 8682 | 0.06% | 8.1 |
| 4 | 50 | SEQ ID NO: 8683 | 0.04% | 5.4 |
| 5 | 29 | SEQ ID NO: 8684 | 0.03% | 4 |
| 6 | 52 | SEQ ID NO: 8685 | 0.02% | 3.24 |
| 7 | 54 | SEQ ID NO: 8686 | 0.02% | 3 |
| 8 | 11 | SEQ ID NO: 8687 | 0.01% | 1.8 |
| 9 | 37 | SEQ ID NO: 8688 | 0.01% | 1.8 |
| 10 | 25 | SEQ ID NO: 8689 | 0.01% | 1.35 |
| 11 | 10 | SEQ ID NO: 8690 | 0.00% | 0.9 |
| 12 | 16 | SEQ ID NO: 8691 | 0.00% | 0.9 |
| 13 | 35 | SEQ ID NO: 8692 | 0.00% | 0.6 |

HLA A3 - 10 mers

| Maximum possible score using this molecule type | | | | 12150 |
|---|---|---|---|---|
| 1 | 49 | SEQ ID NO: 8693 | 0.44% | 54 |
| 2 | 17 | SEQ ID NO: 8694 | 0.22% | 27 |
| 3 | 10 | SEQ ID NO: 8695 | 0.14% | 18 |
| 4 | 16 | SEQ ID NO: 8696 | 0.07% | 9 |
| 5 | 32 | SEQ ID NO: 8697 | 0.04% | 6 |
| 6 | 19 | SEQ ID NO: 8698 | 0.01% | 1.8 |
| 7 | 29 | SEQ ID NO: 8699 | 0.00% | 1.2 |
| 8 | 23 | SEQ ID NO: 8700 | 0.00% | 0.9 |
| 9 | 26 | SEQ ID NO: 8701 | 0.00% | 0.9 |

HLA A24 - 9 mers

| Maximum possible score using this molecule type | | | | 1596.672 |
|---|---|---|---|---|
| 1 | 18 | SEQ ID NO: 8702 | 1.87% | 30 |
| 2 | 24 | SEQ ID NO: 8703 | 0.65% | 10.5 |
| 3 | 9 | SEQ ID NO: 8704 | 0.52% | 8.4 |
| 4 | 12 | SEQ ID NO: 8705 | 0.52% | 8.4 |
| 5 | 28 | SEQ ID NO: 8706 | 0.52% | 8.4 |
| 6 | 42 | SEQ ID NO: 8707 | 0.52% | 8.4 |
| 7 | 57 | SEQ ID NO: 8708 | 0.52% | 8.4 |
| 8 | 66 | SEQ ID NO: 8709 | 0.52% | 8.4 |
| 9 | 55 | SEQ ID NO: 8710 | 0.51% | 8.25 |
| 10 | 0 | SEQ ID NO: 8711 | 0.48% | 7.7 |
| 11 | 22 | SEQ ID NO: 8712 | 0.45% | 7.2 |
| 12 | 10 | SEQ ID NO: 8713 | 0.37% | 6 |
| 13 | 25 | SEQ ID NO: 8714 | 0.37% | 6 |
| 14 | 30 | SEQ ID NO: 8715 | 0.37% | 6 |
| 15 | 19 | SEQ ID NO: 8716 | 0.35% | 5.6 |
| 16 | 40 | SEQ ID NO: 8717 | 0.31% | 5 |
| 17 | 3 | SEQ ID NO: 8718 | 0.30% | 4.8 |
| 18 | 65 | SEQ ID NO: 8719 | 0.30% | 4.8 |
| 19 | 14 | SEQ ID NO: 8720 | 0.27% | 4.32 |
| 20 | 56 | SEQ ID NO: 8721 | 0.25% | 4 |

HLA A24 - 10 mers

| Maximum possible score using this molecule type | | | | 1596.672 |
|---|---|---|---|---|
| 1 | 55 | SEQ ID NO: 8722 | 18.78% | 300 |
| 2 | 18 | SEQ ID NO: 8723 | 2.63% | 42 |
| 3 | 21 | SEQ ID NO: 8724 | 2.25% | 36 |
| 4 | 2 | SEQ ID NO: 8725 | 1.87% | 30 |
| 5 | 24 | SEQ ID NO: 8726 | 1.87% | 30 |
| 6 | 11 | SEQ ID NO: 8727 | 0.52% | 8.4 |
| 7 | 40 | SEQ ID NO: 8728 | 0.52% | 8.4 |
| 8 | 65 | SEQ ID NO: 8729 | 0.42% | 6.72 |
| 9 | 9 | SEQ ID NO: 8730 | 0.37% | 6 |
| 10 | 8 | SEQ ID NO: 8731 | 0.35% | 5.6 |
| 11 | 27 | SEQ ID NO: 8732 | 0.35% | 5.6 |
| 12 | 41 | SEQ ID NO: 8733 | 0.35% | 5.6 |
| 13 | 57 | SEQ ID NO: 8734 | 0.31% | 5 |
| 14 | 17 | SEQ ID NO: 8735 | 0.25% | 4 |
| 15 | 29 | SEQ ID NO: 8736 | 0.25% | 4 |
| 16 | 64 | SEQ ID NO: 8737 | 0.25% | 4 |
| 17 | 16 | SEQ ID NO: 8738 | 0.22% | 3.6 |
| 18 | 10 | SEQ ID NO: 8739 | 0.18% | 3 |
| 19 | 13 | SEQ ID NO: 8740 | 0.18% | 2.88 |
| 20 | 23 | SEQ ID NO: 8741 | 0.08% | 1.4 |

HLA A 0201 - 9 mers

| Maximum possible score using this molecule type | | | | 3925227.1 |
|---|---|---|---|---|
| 1 | 19 | SEQ ID NO: 8742 | 0.03% | 1310.8823136 |
| 2 | 15 | SEQ ID NO: 8743 | 0.02% | 1082.4143022 |
| 3 | 16 | SEQ ID NO: 8744 | 0.02% | 1040.33238624 |
| 4 | 49 | SEQ ID NO: 8745 | 0.00% | 382.536 |
| 5 | 25 | SEQ ID NO: 8746 | 0.00% | 342.863529264 |
| 6 | 56 | SEQ ID NO: 8747 | 0.00% | 63.28397376 |
| 7 | 12 | SEQ ID NO: 8748 | 0.00% | 40.19736105 |
| 8 | 10 | SEQ ID NO: 8749 | 0.00% | 21.3624 |
| 9 | 22 | SEQ ID NO: 8750 | 0.00% | 19.7762418 |
| 10 | 26 | SEQ ID NO: 8751 | 0.00% | 12.6684 |
| 11 | 20 | SEQ ID NO: 8752 | 0.00% | 11.544666 |
| 12 | 37 | SEQ ID NO: 8753 | 0.00% | 10.4328 |
| 13 | 32 | SEQ ID NO: 8754 | 0.00% | 8.4456 |
| 14 | 23 | SEQ ID NO: 8755 | 0.00% | 6.2888049 |
| 15 | 47 | SEQ ID NO: 8756 | 0.00% | 6.0858 |
| 16 | 3 | SEQ ID NO: 8757 | 0.00% | 4.582929078 |
| 17 | 18 | SEQ ID NO: 8758 | 0.00% | 4.4855150505 |
| 18 | 28 | SEQ ID NO: 8759 | 0.00% | 4.2923589 |
| 19 | 62 | SEQ ID NO: 8760 | 0.00% | 2.88098391 |
| 20 | 27 | SEQ ID NO: 8761 | 0.00% | 1.699677 |

HLA A 0201 - 10 mers

| Maximum possible score using this molecule type | | | | 3925227.1 |
|---|---|---|---|---|
| 1 | 17 | SEQ ID NO: 8762 | 0.16% | 6459.14167272 |
| 2 | 19 | SEQ ID NO: 8763 | 0.01% | 607.88448 |
| 3 | 25 | SEQ ID NO: 8764 | 0.00% | 126.83304 |
| 4 | 11 | SEQ ID NO: 8765 | 0.00% | 63.16728165 |
| 5 | 15 | SEQ ID NO: 8766 | 0.00% | 53.54651988 |
| 6 | 37 | SEQ ID NO: 8767 | 0.00% | 28.51632 |
| 7 | 14 | SEQ ID NO: 8768 | 0.00% | 21.8247414 |
| 8 | 29 | SEQ ID NO: 8769 | 0.00% | 21.3624 |
| 9 | 26 | SEQ ID NO: 8770 | 0.00% | 19.42488 |
| 10 | 3 | SEQ ID NO: 8771 | 0.00% | 17.2167282 |
| 11 | 48 | SEQ ID NO: 8772 | 0.00% | 15.7068219 |
| 12 | 12 | SEQ ID NO: 8773 | 0.00% | 9.8581266 |
| 13 | 27 | SEQ ID NO: 8774 | 0.00% | 7.3086111 |
| 14 | 39 | SEQ ID NO: 8775 | 0.00% | 7.10976 |
| 15 | 23 | SEQ ID NO: 8776 | 0.00% | 5.7419523 |
| 16 | 22 | SEQ ID NO: 8777 | 0.00% | 4.599126 |
| 17 | 45 | SEQ ID NO: 8778 | 0.00% | 2.5495155 |
| 18 | 31 | SEQ ID NO: 8779 | 0.00% | 2.52747 |
| 19 | 52 | SEQ ID NO: 8780 | 0.00% | 2.383605 |
| 20 | 20 | SEQ ID NO: 8781 | 0.00% | 2.332847151 |

HLA A 1101 - 9 mers

| Maximum possible score using this molecule type | | | | 36 |
|---|---|---|---|---|
| 1 | 44 | SEQ ID NO: 8782 | 3.33% | 1.2 |

HLA B7 - 9 mers

| | |
|---|---|
| Maximum possible score using this molecule type | 5400 |

HLA B7 - 9 mers

| Maximum possible score using this molecule type | | | | 5400 |
|---|---|---|---|---|
| 1 | 3 | SEQ ID NO: 8783 | 0.37% | 20 |
| 2 | 12 | SEQ ID NO: 8784 | 0.37% | 20 |
| 3 | 22 | SEQ ID NO: 8785 | 0.37% | 20 |
| 4 | 56 | SEQ ID NO: 8786 | 0.37% | 20 |
| 5 | 30 | SEQ ID NO: 8787 | 0.22% | 12 |
| 6 | 9 | SEQ ID NO: 8788 | 0.07% | 4 |
| 7 | 10 | SEQ ID NO: 8789 | 0.07% | 4 |
| 8 | 19 | SEQ ID NO: 8790 | 0.07% | 4 |
| 9 | 25 | SEQ ID NO: 8791 | 0.07% | 4 |
| 10 | 28 | SEQ ID NO: 8792 | 0.07% | 4 |
| 11 | 42 | SEQ ID NO: 8793 | 0.07% | 4 |
| 12 | 65 | SEQ ID NO: 8794 | 0.07% | 4 |
| 13 | 35 | SEQ ID NO: 8795 | 0.05% | 3 |
| 14 | 66 | SEQ ID NO: 8796 | 0.02% | 1.2 |
| 15 | 15 | SEQ ID NO: 8797 | 0.01% | 1 |
| 16 | 47 | SEQ ID NO: 8798 | 0.01% | 1 |
| 17 | 20 | SEQ ID NO: 8799 | 0.01% | 0.6 |
| 18 | 23 | SEQ ID NO: 8800 | 0.00% | 0.5 |
| 19 | 27 | SEQ ID NO: 8801 | 0.00% | 0.5 |

HLA B7 - 10 mers

| Maximum possible score using this molecule type | | | | 5400 |
|---|---|---|---|---|
| 1 | 27 | SEQ ID NO: 8802 | 0.37% | 20 |
| 2 | 8 | SEQ ID NO: 8803 | 0.07% | 4 |
| 3 | 9 | SEQ ID NO: 8804 | 0.07% | 4 |
| 4 | 11 | SEQ ID NO: 8805 | 0.07% | 4 |
| 5 | 17 | SEQ ID NO: 8806 | 0.07% | 4 |
| 6 | 29 | SEQ ID NO: 8807 | 0.07% | 4 |
| 7 | 41 | SEQ ID NO: 8808 | 0.07% | 4 |
| 8 | 52 | SEQ ID NO: 8809 | 0.07% | 4 |
| 9 | 64 | SEQ ID NO: 8810 | 0.07% | 4 |
| 10 | 65 | SEQ ID NO: 8811 | 0.07% | 4 |
| 11 | 3 | SEQ ID NO: 8812 | 0.03% | 2 |
| 12 | 23 | SEQ ID NO: 8813 | 0.03% | 2 |
| 13 | 21 | SEQ ID NO: 8814 | 0.02% | 1.2 |
| 14 | 15 | SEQ ID NO: 8815 | 0.01% | 1 |
| 15 | 35 | SEQ ID NO: 8816 | 0.01% | 0.6 |
| 16 | 39 | SEQ ID NO: 8817 | 0.01% | 0.6 |
| 17 | 12 | SEQ ID NO: 8818 | 0.00% | 0.5 |
| 18 | 22 | SEQ ID NO: 8819 | 0.00% | 0.5 |
| 19 | 45 | SEQ ID NO: 8820 | 0.00% | 0.5 |

TABLE 20
Epitopes for SEQ ID NO: 6046

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 186 | SEQ ID NO: 8821 | 2.22% | 125 |
| 2 | 156 | SEQ ID NO: 8822 | 0.88% | 50 |
| 3 | 14 | SEQ ID NO: 8823 | 0.08% | 4.5 |
| 4 | 0 | SEQ ID NO: 8824 | 0.04% | 2.5 |
| 5 | 29 | SEQ ID NO: 8825 | 0.04% | 2.5 |
| 6 | 85 | SEQ ID NO: 8826 | 0.04% | 2.5 |
| 7 | 168 | SEQ ID NO: 8827 | 0.04% | 2.5 |
| 8 | 133 | SEQ ID NO: 8828 | 0.02% | 1.35 |
| 9 | 111 | SEQ ID NO: 8829 | 0.02% | 1.125 |
| 10 | 61 | SEQ ID NO: 8830 | 0.01% | 1 |
| 11 | 7 | SEQ ID NO: 8831 | 0.01% | 0.9 |
| 12 | 131 | SEQ ID NO: 8832 | 0.01% | 0.9 |
| 13 | 211 | SEQ ID NO: 8833 | 0.01% | 0.625 |
| 14 | 4 | SEQ ID NO: 8834 | 0.00% | 0.5 |
| 15 | 43 | SEQ ID NO: 8835 | 0.00% | 0.5 |
| 16 | 95 | SEQ ID NO: 8836 | 0.00% | 0.5 |
| 17 | 136 | SEQ ID NO: 8837 | 0.00% | 0.5 |

HLA A1 - 10 mers

| Maximum possible score using this molecule type | | | | 5625 |
|---|---|---|---|---|
| 1 | 133 | SEQ ID NO: 8838 | 0.04% | 2.7 |
| 2 | 84 | SEQ ID NO: 8839 | 0.04% | 2.5 |
| 3 | 167 | SEQ ID NO: 8840 | 0.04% | 2.5 |
| 4 | 186 | SEQ ID NO: 8841 | 0.04% | 2.5 |
| 5 | 131 | SEQ ID NO: 8842 | 0.04% | 2.25 |
| 6 | 14 | SEQ ID NO: 8843 | 0.03% | 1.8 |
| 7 | 205 | SEQ ID NO: 8844 | 0.02% | 1.25 |
| 8 | 111 | SEQ ID NO: 8845 | 0.02% | 1.125 |
| 9 | 60 | SEQ ID NO: 8846 | 0.01% | 1 |
| 10 | 188 | SEQ ID NO: 8847 | 0.01% | 0.75 |
| 11 | 211 | SEQ ID NO: 8848 | 0.01% | 0.625 |
| 12 | 26 | SEQ ID NO: 8849 | 0.00% | 0.5 |
| 13 | 94 | SEQ ID NO: 8850 | 0.00% | 0.5 |
| 14 | 135 | SEQ ID NO: 8851 | 0.00% | 0.5 |
| 15 | 168 | SEQ ID NO: 8852 | 0.00% | 0.5 |

HLA A3 - 9 mers

| Maximum possible score using this molecule type | | | | 12150 |
|---|---|---|---|---|
| 1 | 43 | SEQ ID NO: 8853 | 0.24% | 30 |
| 2 | 90 | SEQ ID NO: 8854 | 0.14% | 18 |
| 3 | 148 | SEQ ID NO: 8855 | 0.09% | 12 |
| 4 | 4 | SEQ ID NO: 8856 | 0.05% | 6.75 |
| 5 | 24 | SEQ ID NO: 8857 | 0.04% | 6 |
| 6 | 19 | SEQ ID NO: 8858 | 0.04% | 5.4 |
| 7 | 136 | SEQ ID NO: 8859 | 0.04% | 5.4 |
| 8 | 54 | SEQ ID NO: 8860 | 0.03% | 4.5 |
| 9 | 32 | SEQ ID NO: 8861 | 0.03% | 4 |
| 10 | 14 | SEQ ID NO: 8862 | 0.02% | 3.6 |
| 11 | 59 | SEQ ID NO: 8863 | 0.02% | 3.6 |
| 12 | 88 | SEQ ID NO: 8864 | 0.02% | 3 |
| 13 | 87 | SEQ ID NO: 8865 | 0.02% | 2.7 |
| 14 | 29 | SEQ ID NO: 8866 | 0.01% | 1.8 |
| 15 | 48 | SEQ ID NO: 8867 | 0.01% | 1.8 |
| 16 | 115 | SEQ ID NO: 8868 | 0.01% | 1.8 |
| 17 | 186 | SEQ ID NO: 8869 | 0.01% | 1.8 |
| 18 | 106 | SEQ ID NO: 8870 | 0.01% | 1.5 |
| 19 | 53 | SEQ ID NO: 8871 | 0.01% | 1.35 |
| 20 | 173 | SEQ ID NO: 8872 | 0.00% | 1.2 |

HLA A3 - 10 mers

| Maximum possible score using this molecule type | | | | 12150 |
|---|---|---|---|---|
| 1 | 24 | SEQ ID NO: 8873 | 0.22% | 27 |
| 2 | 54 | SEQ ID NO: 8874 | 0.18% | 22.5 |
| 3 | 135 | SEQ ID NO: 8875 | 0.08% | 10.8 |
| 4 | 51 | SEQ ID NO: 8876 | 0.07% | 9 |
| 5 | 13 | SEQ ID NO: 8877 | 0.06% | 8.1 |
| 6 | 26 | SEQ ID NO: 8878 | 0.04% | 6 |
| 7 | 31 | SEQ ID NO: 8879 | 0.04% | 6 |
| 8 | 90 | SEQ ID NO: 8880 | 0.04% | 6 |
| 9 | 43 | SEQ ID NO: 8881 | 0.03% | 4.5 |
| 10 | 19 | SEQ ID NO: 8882 | 0.03% | 4.05 |
| 11 | 169 | SEQ ID NO: 8883 | 0.02% | 3 |
| 12 | 87 | SEQ ID NO: 8884 | 0.02% | 2.7 |
| 13 | 84 | SEQ ID NO: 8885 | 0.01% | 1.8 |
| 14 | 88 | SEQ ID NO: 8886 | 0.01% | 1.8 |
| 15 | 94 | SEQ ID NO: 8887 | 0.01% | 1.8 |
| 16 | 64 | SEQ ID NO: 8888 | 0.00% | 1.2 |
| 17 | 131 | SEQ ID NO: 8889 | 0.00% | 1.2 |
| 18 | 99 | SEQ ID NO: 8890 | 0.00% | 1 |
| 19 | 53 | SEQ ID NO: 8891 | 0.00% | 0.9 |
| 20 | 85 | SEQ ID NO: 8892 | 0.00% | 0.9 |

HLA A24 - 9 mers

| Maximum possible score using this molecule type | | | | 1596.672 |
|---|---|---|---|---|
| 1 | 196 | SEQ ID NO: 8893 | 27.55% | 440 |
| 2 | 44 | SEQ ID NO: 8894 | 18.78% | 300 |
| 3 | 36 | SEQ ID NO: 8895 | 12.52% | 200 |
| 4 | 92 | SEQ ID NO: 8896 | 12.52% | 200 |
| 5 | 109 | SEQ ID NO: 8897 | 2.70% | 43.2 |
| 6 | 25 | SEQ ID NO: 8898 | 1.87% | 30 |
| 7 | 93 | SEQ ID NO: 8899 | 1.12% | 18 |
| 8 | 12 | SEQ ID NO: 8900 | 0.75% | 12 |
| 9 | 123 | SEQ ID NO: 8901 | 0.70% | 11.2 |
| 10 | 7 | SEQ ID NO: 8902 | 0.64% | 10.368 |
| 11 | 17 | SEQ ID NO: 8903 | 0.52% | 8.4 |
| 12 | 139 | SEQ ID NO: 8904 | 0.52% | 8.4 |
| 13 | 193 | SEQ ID NO: 8905 | 0.46% | 7.5 |
| 14 | 6 | SEQ ID NO: 8906 | 0.45% | 7.2 |
| 15 | 19 | SEQ ID NO: 8907 | 0.45% | 7.2 |
| 16 | 110 | SEQ ID NO: 8908 | 0.45% | 7.2 |
| 17 | 114 | SEQ ID NO: 8909 | 0.45% | 7.2 |
| 18 | 210 | SEQ ID NO: 8910 | 0.45% | 7.2 |
| 19 | 46 | SEQ ID NO: 8911 | 0.42% | 6.72 |
| 20 | 52 | SEQ ID NO: 8912 | 0.37% | 6 |

HLA A24 - 10 mers

| Maximum possible score using this molecule type | | | | 1596.672 |
|---|---|---|---|---|
| 1 | 92 | SEQ ID NO: 8913 | 7.51% | 120 |
| 2 | 42 | SEQ ID NO: 8914 | 2.63% | 42 |
| 3 | 109 | SEQ ID NO: 8915 | 2.25% | 36 |
| 4 | 23 | SEQ ID NO: 8916 | 1.87% | 30 |
| 5 | 34 | SEQ ID NO: 8917 | 0.75% | 12 |
| 6 | 6 | SEQ ID NO: 8918 | 0.64% | 10.368 |
| 7 | 45 | SEQ ID NO: 8919 | 0.63% | 10.08 |
| 8 | 196 | SEQ ID NO: 8920 | 0.62% | 10 |
| 9 | 44 | SEQ ID NO: 8921 | 0.56% | 9 |
| 10 | 40 | SEQ ID NO: 8922 | 0.55% | 8.8 |
| 11 | 62 | SEQ ID NO: 8923 | 0.46% | 7.5 |
| 12 | 193 | SEQ ID NO: 8924 | 0.46% | 7.5 |
| 13 | 18 | SEQ ID NO: 8925 | 0.45% | 7.2 |
| 14 | 113 | SEQ ID NO: 8926 | 0.45% | 7.2 |
| 15 | 56 | SEQ ID NO: 8927 | 0.37% | 6 |
| 16 | 176 | SEQ ID NO: 8928 | 0.37% | 6 |
| 17 | 16 | SEQ ID NO: 8929 | 0.35% | 5.6 |
| 18 | 138 | SEQ ID NO: 8930 | 0.35% | 5.6 |
| 19 | 127 | SEQ ID NO: 8931 | 0.33% | 5.28 |
| 20 | 36 | SEQ ID NO: 8932 | 0.31% | 5 |

HLA A 0201 - 9 mers

| Maximum possible score using this molecule type | | | | 3925227.1 |
|---|---|---|---|---|
| 1 | 13 | SEQ ID NO: 8933 | 0.04% | 1793.676528 |
| 2 | 87 | SEQ ID NO: 8934 | 0.03% | 1415.3832 |
| 3 | 24 | SEQ ID NO: 8935 | 0.01% | 618.0996816 |
| 4 | 19 | SEQ ID NO: 8936 | 0.00% | 223.23708 |
| 5 | 12 | SEQ ID NO: 8937 | 0.00% | 210.36400875 |
| 6 | 51 | SEQ ID NO: 8938 | 0.00% | 198.30859992 |
| 7 | 53 | SEQ ID NO: 8939 | 0.00% | 194.477328 |
| 8 | 88 | SEQ ID NO: 8940 | 0.00% | 180.58536756 |
| 9 | 106 | SEQ ID NO: 8941 | 0.00% | 169.74828 |
| 10 | 54 | SEQ ID NO: 8942 | 0.00% | 70.09848 |
| 11 | 59 | SEQ ID NO: 8943 | 0.00% | 43.42032 |
| 12 | 94 | SEQ ID NO: 8944 | 0.00% | 41.792058 |
| 13 | 20 | SEQ ID NO: 8945 | 0.00% | 37.46088108 |
| 14 | 63 | SEQ ID NO: 8946 | 0.00% | 35.73520902 |
| 15 | 22 | SEQ ID NO: 8947 | 0.00% | 20.5916435109 |
| 16 | 47 | SEQ ID NO: 8948 | 0.00% | 12.233222865 |
| 17 | 66 | SEQ ID NO: 8949 | 0.00% | 12.2199 |
| 18 | 56 | SEQ ID NO: 8950 | 0.00% | 11.486706 |
| 19 | 67 | SEQ ID NO: 8951 | 0.00% | 6.416172 |
| 20 | 117 | SEQ ID NO: 8952 | 0.00% | 5.827464 |

HLA A 0201 - 10 mers

| Maximum possible score using this molecule type | | | | 3925227.1 |
|---|---|---|---|---|
| 1 | 43 | SEQ ID NO: 8953 | 0.10% | 3977.8497792 |
| 2 | 24 | SEQ ID NO: 8954 | 0.02% | 836.2525104 |
| 3 | 51 | SEQ ID NO: 8955 | 0.02% | 815.616432 |
| 4 | 49 | SEQ ID NO: 8956 | 0.01% | 660.3245145 |
| 5 | 19 | SEQ ID NO: 8957 | 0.00% | 251.837856 |
| 6 | 59 | SEQ ID NO: 8958 | 0.00% | 159.9696 |
| 7 | 12 | SEQ ID NO: 8959 | 0.00% | 155.245377 |
| 8 | 45 | SEQ ID NO: 8960 | 0.00% | 141.1974531 |
| 9 | 21 | SEQ ID NO: 8961 | 0.00% | 117.22672269 |
| 10 | 53 | SEQ ID NO: 8962 | 0.00% | 84.55536 |
| 11 | 87 | SEQ ID NO: 8963 | 0.00% | 65.5671672 |
| 12 | 13 | SEQ ID NO: 8964 | 0.00% | 64.88888616 |
| 13 | 153 | SEQ ID NO: 8965 | 0.00% | 49.13352 |
| 14 | 178 | SEQ ID NO: 8966 | 0.00% | 26.082 |
| 15 | 18 | SEQ ID NO: 8967 | 0.00% | 24.802259691 |
| 16 | 116 | SEQ ID NO: 8968 | 0.00% | 21.5616168 |
| 17 | 65 | SEQ ID NO: 8969 | 0.00% | 20.77383 |
| 18 | 86 | SEQ ID NO: 8970 | 0.00% | 15.7068219 |
| 19 | 27 | SEQ ID NO: 8971 | 0.00% | 12.3159135 |
| 20 | 46 | SEQ ID NO: 8972 | 0.00% | 11.45624789925 |

HLA A 1101 - 9 mers

| Maximum possible score using this molecule type | | | | 36 |
|---|---|---|---|---|
| 1 | 4 | SEQ ID NO: 8973 | 12.5% | 4.5 |
| 2 | 136 | SEQ ID NO: 8974 | 3.33% | 1.2 |
| 3 | 156 | SEQ ID NO: 8975 | 3.33% | 1.2 |
| 4 | 140 | SEQ ID NO: 8976 | 1.66% | 0.6 |

HLA A 1101 - 10 mers

| Maximum possible score using this molecule type | | | | 36 |
|---|---|---|---|---|
| 1 | 169 | SEQ ID NO: 8977 | 5.55% | 2 |
| 2 | 94 | SEQ ID NO: 8978 | 3.33% | 1.2 |

HLA B7 - 9 mers

| Maximum possible score using this molecule type | | | | 5400 |
|---|---|---|---|---|
| 1 | 146 | SEQ ID NO: 8979 | 0.74% | 40 |
| 2 | 154 | SEQ ID NO: 8980 | 0.74% | 40 |
| 3 | 80 | SEQ ID NO: 8981 | 0.66% | 36 |
| 4 | 139 | SEQ ID NO: 8982 | 0.33% | 18 |
| 5 | 83 | SEQ ID NO: 8983 | 0.22% | 12 |
| 6 | 209 | SEQ ID NO: 8984 | 0.22% | 12 |
| 7 | 7 | SEQ ID NO: 8985 | 0.11% | 6 |
| 8 | 3 | SEQ ID NO: 8986 | 0.07% | 4 |
| 9 | 6 | SEQ ID NO: 8987 | 0.07% | 4 |
| 10 | 12 | SEQ ID NO: 8988 | 0.07% | 4 |
| 11 | 19 | SEQ ID NO: 8989 | 0.07% | 4 |
| 12 | 24 | SEQ ID NO: 8990 | 0.07% | 4 |
| 13 | 38 | SEQ ID NO: 8991 | 0.07% | 4 |
| 14 | 46 | SEQ ID NO: 8992 | 0.07% | 4 |
| 15 | 56 | SEQ ID NO: 8993 | 0.07% | 4 |
| 16 | 110 | SEQ ID NO: 8994 | 0.07% | 4 |
| 17 | 114 | SEQ ID NO: 8995 | 0.07% | 4 |
| 18 | 123 | SEQ ID NO: 8996 | 0.07% | 4 |
| 19 | 129 | SEQ ID NO: 8997 | 0.07% | 4 |
| 20 | 166 | SEQ ID NO: 8998 | 0.07% | 4 |

HLA B7 - 10 mers

| Maximum possible score using this molecule type | | | | 5400 |
|---|---|---|---|---|
| 1 | 56 | SEQ ID NO: 8999 | 1.48% | 80 |
| 2 | 40 | SEQ ID NO: 9000 | 0.74% | 40 |
| 3 | 127 | SEQ ID NO: 9001 | 0.74% | 40 |
| 4 | 170 | SEQ ID NO: 9002 | 0.74% | 40 |
| 5 | 140 | SEQ ID NO: 9003 | 0.27% | 15 |
| 6 | 35 | SEQ ID NO: 9004 | 0.22% | 12 |
| 7 | 79 | SEQ ID NO: 9005 | 0.22% | 12 |
| 8 | 82 | SEQ ID NO: 9006 | 0.22% | 12 |
| 9 | 208 | SEQ ID NO: 9007 | 0.22% | 12 |
| 10 | 209 | SEQ ID NO: 9008 | 0.22% | 12 |
| 11 | 80 | SEQ ID NO: 9009 | 0.16% | 9 |
| 12 | 129 | SEQ ID NO: 9010 | 0.14% | 8 |
| 13 | 138 | SEQ ID NO: 9011 | 0.11% | 6 |
| 14 | 73 | SEQ ID NO: 9012 | 0.09% | 5 |
| 15 | 2 | SEQ ID NO: 9013 | 0.07% | 4 |
| 16 | 5 | SEQ ID NO: 9014 | 0.07% | 4 |
| 17 | 6 | SEQ ID NO: 9015 | 0.07% | 4 |
| 18 | 16 | SEQ ID NO: 9016 | 0.07% | 4 |
| 19 | 18 | SEQ ID NO: 9017 | 0.07% | 4 |
| 20 | 24 | SEQ ID NO: 9018 | 0.07% | 4 |

TABLE 21
Epitopes for SEQ ID NO: 6047

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 53 | SEQ ID NO: 9019 | 2% | 112.5 |
| 2 | 10 | SEQ ID NO: 9020 | 0.08% | 4.5 |
| 3 | 33 | SEQ ID NO: 9021 | 0.02% | 1.5 |
| 4 | 3 | SEQ ID NO: 9022 | 0.00% | 0.5 |
| 5 | 27 | SEQ ID NO: 9023 | 0.00% | 0.5 |
| 6 | 29 | SEQ ID NO: 9024 | 0.00% | 0.5 |

HLA A1 - 10 mers

| Maximum possible score using this molecule type | | | | 5625 |
|---|---|---|---|---|
| 1 | 10 | SEQ ID NO: 9025 | 0.8% | 45 |
| 2 | 52 | SEQ ID NO: 9026 | 0.2% | 11.25 |
| 3 | 50 | SEQ ID NO: 9027 | 0.04% | 2.5 |
| 4 | 32 | SEQ ID NO: 9028 | 0.02% | 1.5 |
| 5 | 48 | SEQ ID NO: 9029 | 0.02% | 1.35 |
| 6 | 27 | SEQ ID NO: 9030 | 0.00% | 0.5 |

HLA A3 - 9 mers

| Maximum possible score using this molecule type | | | | 12150 |
|---|---|---|---|---|
| 1 | 38 | SEQ ID NO: 9031 | 1.85% | 225 |
| 2 | 17 | SEQ ID NO: 9032 | 0.02% | 3.6 |
| 3 | 2 | SEQ ID NO: 9033 | 0.02% | 2.7 |
| 4 | 37 | SEQ ID NO: 9034 | 0.01% | 1.8 |
| 5 | 27 | SEQ ID NO: 9035 | 0.01% | 1.35 |
| 6 | 13 | SEQ ID NO: 9036 | 0.00% | 0.675 |
| 7 | 14 | SEQ ID NO: 9037 | 0.00% | 0.6 |

HLA A3 - 10 mers

| Maximum possible score using this molecule type | | | | 12150 |
|---|---|---|---|---|
| 1 | 13 | SEQ ID NO: 9038 | 0.04% | 6 |
| 2 | 37 | SEQ ID NO: 9039 | 0.01% | 2.025 |
| 3 | 2 | SEQ ID NO: 9040 | 0.00% | 0.9 |
| 4 | 19 | SEQ ID NO: 9041 | 0.00% | 0.675 |
| 5 | 16 | SEQ ID NO: 9042 | 0.00% | 0.54 |

HLA A24 - 9 mers

| Maximum possible score using this molecule type | | | | 1596.672 |
|---|---|---|---|---|
| 1 | 20 | SEQ ID NO: 9043 | 1.25% | 20 |
| 2 | 6 | SEQ ID NO: 9044 | 0.52% | 8.4 |
| 3 | 5 | SEQ ID NO: 9045 | 0.51% | 8.25 |
| 4 | 35 | SEQ ID NO: 9046 | 0.36% | 5.76 |
| 5 | 31 | SEQ ID NO: 9047 | 0.35% | 5.6 |
| 6 | 43 | SEQ ID NO: 9048 | 0.27% | 4.4 |
| 7 | 13 | SEQ ID NO: 9049 | 0.26% | 4.2 |
| 8 | 32 | SEQ ID NO: 9050 | 0.21% | 3.36 |
| 9 | 2 | SEQ ID NO: 9051 | 0.11% | 1.8 |
| 10 | 9 | SEQ ID NO: 9052 | 0.10% | 1.68 |
| 11 | 8 | SEQ ID NO: 9053 | 0.09% | 1.5 |
| 12 | 15 | SEQ ID NO: 9054 | 0.09% | 1.5 |
| 13 | 23 | SEQ ID NO: 9055 | 0.09% | 1.5 |
| 14 | 27 | SEQ ID NO: 9056 | 0.08% | 1.4 |
| 15 | 24 | SEQ ID NO: 9057 | 0.07% | 1.2 |
| 16 | 7 | SEQ ID NO: 9058 | 0.06% | 1 |
| 17 | 17 | SEQ ID NO: 9059 | 0.06% | 1 |
| 18 | 10 | SEQ ID NO: 9060 | 0.05% | 0.9 |
| 19 | 39 | SEQ ID NO: 9061 | 0.04% | 0.792 |
| 20 | 47 | SEQ ID NO: 9062 | 0.04% | 0.792 |

HLA A24 - 10 mers

| Maximum possible score using this molecule type | | | | 1596.672 |
|---|---|---|---|---|
| 1 | 5 | SEQ ID NO: 9063 | 2.63% | 42 |
| 2 | 34 | SEQ ID NO: 9064 | 0.54% | 8.64 |
| 3 | 30 | SEQ ID NO: 9065 | 0.52% | 8.4 |
| 4 | 19 | SEQ ID NO: 9066 | 0.50% | 8 |
| 5 | 50 | SEQ ID NO: 9067 | 0.33% | 5.28 |
| 6 | 12 | SEQ ID NO: 9068 | 0.26% | 4.2 |
| 7 | 31 | SEQ ID NO: 9069 | 0.21% | 3.36 |
| 8 | 26 | SEQ ID NO: 9070 | 0.15% | 2.52 |
| 9 | 8 | SEQ ID NO: 9071 | 0.13% | 2.1 |
| 10 | 22 | SEQ ID NO: 9072 | 0.12% | 2 |
| 11 | 23 | SEQ ID NO: 9073 | 0.11% | 1.8 |
| 12 | 6 | SEQ ID NO: 9074 | 0.09% | 1.5 |
| 13 | 14 | SEQ ID NO: 9075 | 0.09% | 1.5 |
| 14 | 16 | SEQ ID NO: 9076 | 0.09% | 1.5 |
| 15 | 7 | SEQ ID NO: 9077 | 0.06% | 1 |
| 16 | 48 | SEQ ID NO: 9078 | 0.04% | 0.75 |
| 17 | 0 | SEQ ID NO: 9079 | 0.04% | 0.72 |
| 18 | 9 | SEQ ID NO: 9080 | 0.04% | 0.72 |
| 19 | 47 | SEQ ID NO: 9081 | 0.04% | 0.66 |
| 20 | 39 | SEQ ID NO: 9082 | 0.03% | 0.6 |

HLA A 0201 - 9 mers

| Maximum possible score using this molecule type | | | | 3925227.1 |
|---|---|---|---|---|
| 1 | 15 | SEQ ID NO: 9083 | 0.00% | 14.1442686 |
| 2 | 27 | SEQ ID NO: 9084 | 0.00% | 9.598176 |
| 3 | 22 | SEQ ID NO: 9085 | 0.00% | 9.5634 |
| 4 | 9 | SEQ ID NO: 9086 | 0.00% | 5.546246013 |
| 5 | 2 | SEQ ID NO: 9087 | 0.00% | 5.526462816 |
| 6 | 24 | SEQ ID NO: 9088 | 0.00% | 4.88163753 |
| 7 | 17 | SEQ ID NO: 9089 | 0.00% | 3.699285408 |
| 8 | 31 | SEQ ID NO: 9090 | 0.00% | 2.29699206 |
| 9 | 6 | SEQ ID NO: 9091 | 0.00% | 2.0016040674 |
| 10 | 7 | SEQ ID NO: 9092 | 0.00% | 0.91287 |
| 11 | 49 | SEQ ID NO: 9093 | 0.00% | 0.71805678 |
| 12 | 16 | SEQ ID NO: 9094 | 0.00% | 0.6694257042 |
| 13 | 12 | SEQ ID NO: 9095 | 0.00% | 0.6539828625 |

HLA A 0201 - 10 mers

| Maximum possible score using this molecule type | | | | 3925227.1 |
|---|---|---|---|---|
| 1 | 16 | SEQ ID NO: 9096 | 0.00% | 34.28765802 |
| 2 | 19 | SEQ ID NO: 9097 | 0.00% | 18.9368775 |
| 3 | 14 | SEQ ID NO: 9098 | 0.00% | 14.1442686 |
| 4 | 27 | SEQ ID NO: 9099 | 0.00% | 11.406528 |
| 5 | 26 | SEQ ID NO: 9100 | 0.00% | 10.9304361558 |
| 6 | 34 | SEQ ID NO: 9101 | 0.00% | 5.580927 |
| 7 | 6 | SEQ ID NO: 9102 | 0.00% | 4.865742 |
| 8 | 9 | SEQ ID NO: 9103 | 0.00% | 2.64106953 |
| 9 | 50 | SEQ ID NO: 9104 | 0.00% | 2.6275752 |
| 10 | 30 | SEQ ID NO: 9105 | 0.00% | 2.29699206 |
| 11 | 7 | SEQ ID NO: 9106 | 0.00% | 0.86083641 |
| 12 | 42 | SEQ ID NO: 9107 | 0.00% | 0.7049592 |
| 13 | 22 | SEQ ID NO: 9108 | 0.00% | 0.6628440357 |
| 14 | 2 | SEQ ID NO: 9109 | 0.00% | 0.6530644656 |

HLA A 1101 - 9 mers

| Maximum possible score using this molecule type | | | | 36 |
|---|---|---|---|---|
| 1 | 37 | SEQ ID NO: 9110 | 15% | 5.4 |
| 2 | 38 | SEQ ID NO: 9111 | 2.22% | 0.8 |

HLA A 1101 - 10 mers

| Maximum possible score using this molecule type | | | | 36 |
|---|---|---|---|---|
| 1 | 37 | SEQ ID NO: 9112 | 7.5% | 2.7 |

HLA B7 - 9 mers

| Maximum possible score using this molecule type | | | | 5400 |
|---|---|---|---|---|
| 1 | 35 | SEQ ID NO: 9113 | 3.70% | 200 |
| 2 | 17 | SEQ ID NO: 9114 | 0.11% | 6 |
| 3 | 6 | SEQ ID NO: 9115 | 0.07% | 4 |
| 4 | 20 | SEQ ID NO: 9116 | 0.07% | 4 |
| 5 | 31 | SEQ ID NO: 9117 | 0.07% | 4 |
| 6 | 43 | SEQ ID NO: 9118 | 0.07% | 4 |
| 7 | 7 | SEQ ID NO: 9119 | 0.03% | 2 |
| 8 | 23 | SEQ ID NO: 9120 | 0.02% | 1.2 |
| 9 | 24 | SEQ ID NO: 9121 | 0.02% | 1.2 |
| 10 | 10 | SEQ ID NO: 9122 | 0.01% | 0.9 |

HLA B7 - 10 mers

| Maximum possible score using this molecule type | | | | 5400 |
|---|---|---|---|---|
| 1 | 35 | SEQ ID NO: 9123 | 0.09% | 5 |
| 2 | 19 | SEQ ID NO: 9124 | 0.07% | 4 |
| 3 | 30 | SEQ ID NO: 9125 | 0.07% | 4 |
| 4 | 34 | SEQ ID NO: 9126 | 0.07% | 4 |
| 5 | 7 | SEQ ID NO: 9127 | 0.03% | 2 |
| 6 | 16 | SEQ ID NO: 9128 | 0.03% | 1.8 |
| 7 | 23 | SEQ ID NO: 9129 | 0.02% | 1.2 |
| 8 | 50 | SEQ ID NO: 9130 | 0.02% | 1.2 |
| 9 | 9 | SEQ ID NO: 9131 | 0.01% | 1 |

TABLE 22
Epitopes for SEQ ID NO: 6048

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 66 | SEQ ID NO: 9132 | 0.44% | 25 |
| 2 | 80 | SEQ ID NO: 9133 | 0.08% | 5 |
| 3 | 93 | SEQ ID NO: 9134 | 0.04% | 2.7 |
| 4 | 11 | SEQ ID NO: 9135 | 0.04% | 2.5 |
| 5 | 89 | SEQ ID NO: 9136 | 0.04% | 2.25 |
| 6 | 48 | SEQ ID NO: 9137 | 0.01% | 1 |
| 7 | 3 | SEQ ID NO: 9138 | 0.00% | 0.5 |
| 8 | 9 | SEQ ID NO: 9139 | 0.00% | 0.5 |
| 9 | 56 | SEQ ID NO: 9140 | 0.00% | 0.5 |
| 10 | 101 | SEQ ID NO: 9141 | 0.00% | 0.5 |
| 11 | 106 | SEQ ID NO: 9142 | 0.00% | 0.5 |
| 12 | 110 | SEQ ID NO: 9143 | 0.00% | 0.5 |

HLA A1 - 10 mers

| Maximum possible score using this molecule type | | | | 5625 |
|---|---|---|---|---|
| 1 | 30 | SEQ ID NO: 9144 | 0.4% | 22.5 |
| 2 | 88 | SEQ ID NO: 9145 | 0.12% | 6.75 |
| 3 | 48 | SEQ ID NO: 9146 | 0.04% | 2.5 |
| 4 | 55 | SEQ ID NO: 9147 | 0.02% | 1.25 |
| 5 | 13 | SEQ ID NO: 9148 | 0.01% | 0.9 |
| 6 | 79 | SEQ ID NO: 9149 | 0.01% | 0.75 |
| 7 | 93 | SEQ ID NO: 9150 | 0.01% | 0.675 |
| 8 | 2 | SEQ ID NO: 9151 | 0.00% | 0.5 |
| 9 | 8 | SEQ ID NO: 9152 | 0.00% | 0.5 |
| 10 | 65 | SEQ ID NO: 9153 | 0.00% | 0.5 |
| 11 | 66 | SEQ ID NO: 9154 | 0.00% | 0.5 |
| 12 | 80 | SEQ ID NO: 9155 | 0.00% | 0.5 |
| 13 | 105 | SEQ ID NO: 9156 | 0.00% | 0.5 |
| 14 | 109 | SEQ ID NO: 9157 | 0.00% | 0.5 |

HLA A3 - 9 mers

| Maximum possible score using this molecule type | | | | 12150 |
|---|---|---|---|---|
| 1 | 109 | SEQ ID NO: 9158 | 0.74% | 90 |
| 2 | 3 | SEQ ID NO: 9159 | 0.24% | 30 |
| 3 | 111 | SEQ ID NO: 9160 | 0.12% | 15 |
| 4 | 106 | SEQ ID NO: 9161 | 0.07% | 9 |
| 5 | 95 | SEQ ID NO: 9162 | 0.05% | 6.075 |
| 6 | 101 | SEQ ID NO: 9163 | 0.04% | 6 |
| 7 | 110 | SEQ ID NO: 9164 | 0.02% | 3.6 |
| 8 | 84 | SEQ ID NO: 9165 | 0.02% | 3 |
| 9 | 80 | SEQ ID NO: 9166 | 0.02% | 2.7 |
| 10 | 37 | SEQ ID NO: 9167 | 0.01% | 2.25 |
| 11 | 9 | SEQ ID NO: 9168 | 0.01% | 2 |
| 12 | 54 | SEQ ID NO: 9169 | 0.01% | 2 |
| 13 | 99 | SEQ ID NO: 9170 | 0.01% | 1.35 |
| 14 | 1 | SEQ ID NO: 9171 | 0.01% | 1.215 |
| 15 | 11 | SEQ ID NO: 9172 | 0.00% | 0.9 |
| 16 | 15 | SEQ ID NO: 9173 | 0.00% | 0.9 |
| 17 | 69 | SEQ ID NO: 9174 | 0.00% | 0.6 |
| 18 | 5 | SEQ ID NO: 9175 | 0.00% | 0.54 |
| 19 | 103 | SEQ ID NO: 9176 | 0.00% | 0.54 |

HLA A3 - 10 mers

| Maximum possible score using this molecule type | | | | 12150 |
|---|---|---|---|---|
| 1 | 75 | SEQ ID NO: 9177 | 0.49% | 60 |
| 2 | 109 | SEQ ID NO: 9178 | 0.29% | 36 |
| 3 | 22 | SEQ ID NO: 9179 | 0.14% | 18 |
| 4 | 15 | SEQ ID NO: 9180 | 0.04% | 6 |
| 5 | 110 | SEQ ID NO: 9181 | 0.01% | 2.25 |
| 6 | 95 | SEQ ID NO: 9182 | 0.01% | 1.8 |
| 7 | 101 | SEQ ID NO: 9183 | 0.01% | 1.35 |
| 8 | 43 | SEQ ID NO: 9184 | 0.00% | 1 |
| 9 | 2 | SEQ ID NO: 9185 | 0.00% | 0.9 |
| 10 | 5 | SEQ ID NO: 9186 | 0.00% | 0.9 |
| 11 | 7 | SEQ ID NO: 9187 | 0.00% | 0.9 |
| 12 | 107 | SEQ ID NO: 9188 | 0.00% | 0.9 |
| 13 | 102 | SEQ ID NO: 9189 | 0.00% | 0.81 |
| 14 | 3 | SEQ ID NO: 9190 | 0.00% | 0.75 |
| 15 | 8 | SEQ ID NO: 9191 | 0.00% | 0.6 |
| 16 | 103 | SEQ ID NO: 9192 | 0.00% | 0.54 |

HLA A24 - 9 mers

| Maximum possible score using this molecule type | | | | 1596.672 |
|---|---|---|---|---|
| 1 | 88 | SEQ ID NO: 9193 | 1.66% | 26.6112 |
| 2 | 77 | SEQ ID NO: 9194 | 0.77% | 12.32 |
| 3 | 18 | SEQ ID NO: 9195 | 0.56% | 9 |
| 4 | 108 | SEQ ID NO: 9196 | 0.56% | 9 |
| 5 | 92 | SEQ ID NO: 9197 | 0.54% | 8.64 |
| 6 | 96 | SEQ ID NO: 9198 | 0.54% | 8.64 |
| 7 | 73 | SEQ ID NO: 9199 | 0.46% | 7.5 |
| 8 | 40 | SEQ ID NO: 9200 | 0.45% | 7.2 |
| 9 | 104 | SEQ ID NO: 9201 | 0.42% | 6.72 |
| 10 | 8 | SEQ ID NO: 9202 | 0.41% | 6.6 |
| 11 | 21 | SEQ ID NO: 9203 | 0.37% | 6 |
| 12 | 102 | SEQ ID NO: 9204 | 0.37% | 6 |
| 13 | 22 | SEQ ID NO: 9205 | 0.25% | 4 |
| 14 | 68 | SEQ ID NO: 9206 | 0.25% | 4 |
| 15 | 106 | SEQ ID NO: 9207 | 0.22% | 3.6 |
| 16 | 1 | SEQ ID NO: 9208 | 0.18% | 3 |
| 17 | 79 | SEQ ID NO: 9209 | 0.18% | 3 |
| 18 | 93 | SEQ ID NO: 9210 | 0.18% | 3 |
| 19 | 101 | SEQ ID NO: 9211 | 0.18% | 3 |
| 20 | 37 | SEQ ID NO: 9212 | 0.15% | 2.4 |

HLA A24 - 10 mers

| Maximum possible score using this molecule type | | | | 1596.672 |
|---|---|---|---|---|
| 1 | 100 | SEQ ID NO: 9213 | 0.93% | 15 |
| 2 | 18 | SEQ ID NO: 9214 | 0.78% | 12.6 |
| 3 | 98 | SEQ ID NO: 9215 | 0.52% | 8.4 |
| 4 | 73 | SEQ ID NO: 9216 | 0.46% | 7.5 |
| 5 | 91 | SEQ ID NO: 9217 | 0.45% | 7.2 |
| 6 | 103 | SEQ ID NO: 9218 | 0.42% | 6.72 |
| 7 | 7 | SEQ ID NO: 9219 | 0.41% | 6.6 |
| 8 | 21 | SEQ ID NO: 9220 | 0.37% | 6 |
| 9 | 46 | SEQ ID NO: 9221 | 0.37% | 6 |
| 10 | 93 | SEQ ID NO: 9222 | 0.37% | 6 |
| 11 | 96 | SEQ ID NO: 9223 | 0.37% | 6 |
| 12 | 101 | SEQ ID NO: 9224 | 0.37% | 6 |
| 13 | 77 | SEQ ID NO: 9225 | 0.25% | 4 |
| 14 | 92 | SEQ ID NO: 9226 | 0.22% | 3.6 |
| 15 | 105 | SEQ ID NO: 9227 | 0.22% | 3.6 |
| 16 | 2 | SEQ ID NO: 9228 | 0.18% | 3 |
| 17 | 53 | SEQ ID NO: 9229 | 0.18% | 3 |
| 18 | 36 | SEQ ID NO: 9230 | 0.12% | 2 |
| 19 | 55 | SEQ ID NO: 9231 | 0.12% | 2 |
| 20 | 102 | SEQ ID NO: 9232 | 0.11% | 1.8 |

HLA A 0201 - 9 mers

| Maximum possible score using this molecule type | | | | 3925227.1 |
|---|---|---|---|---|
| 1 | 84 | SEQ ID NO: 9233 | 0.01% | 441.342216 |
| 2 | 102 | SEQ ID NO: 9234 | 0.00% | 63.16728165 |
| 3 | 107 | SEQ ID NO: 9235 | 0.00% | 51.882640425 |
| 4 | 1 | SEQ ID NO: 9236 | 0.00% | 43.8816609 |
| 5 | 95 | SEQ ID NO: 9237 | 0.00% | 33.40165248 |
| 6 | 2 | SEQ ID NO: 9238 | 0.00% | 24.66305226 |
| 7 | 92 | SEQ ID NO: 9239 | 0.00% | 22.64458905 |
| 8 | 103 | SEQ ID NO: 9240 | 0.00% | 20.70206586 |
| 9 | 47 | SEQ ID NO: 9241 | 0.00% | 11.175953184 |
| 10 | 94 | SEQ ID NO: 9242 | 0.00% | 8.452983 |
| 11 | 15 | SEQ ID NO: 9243 | 0.00% | 8.1793152 |
| 12 | 8 | SEQ ID NO: 9244 | 0.00% | 4.993461 |
| 13 | 5 | SEQ ID NO: 9245 | 0.00% | 4.57284528 |
| 14 | 99 | SEQ ID NO: 9246 | 0.00% | 3.999468528 |
| 15 | 105 | SEQ ID NO: 9247 | 0.00% | 2.231322 |
| 16 | 20 | SEQ ID NO: 9248 | 0.00% | 1.3524 |
| 17 | 62 | SEQ ID NO: 9249 | 0.00% | 0.8631693 |
| 18 | 6 | SEQ ID NO: 9250 | 0.00% | 0.824619 |
| 19 | 57 | SEQ ID NO: 9251 | 0.00% | 0.72105 |
| 20 | 58 | SEQ ID NO: 9252 | 0.00% | 0.7147572 |

HLA A 0201 - 10 mers

| Maximum possible score using this molecule type | | | | 3925227.1 |
|---|---|---|---|---|
| 1 | 101 | SEQ ID NO: 9253 | 0.03% | 1243.078056 |
| 2 | 3 | SEQ ID NO: 9254 | 0.01% | 592.944462 |
| 3 | 106 | SEQ ID NO: 9255 | 0.00% | 94.2678 |
| 4 | 5 | SEQ ID NO: 9256 | 0.00% | 43.42032 |
| 5 | 107 | SEQ ID NO: 9257 | 0.00% | 33.30332334 |
| 6 | 102 | SEQ ID NO: 9258 | 0.00% | 32.53181778 |
| 7 | 54 | SEQ ID NO: 9259 | 0.00% | 27.324 |
| 8 | 7 | SEQ ID NO: 9260 | 0.00% | 21.3624 |
| 9 | 1 | SEQ ID NO: 9261 | 0.00% | 13.723479 |
| 10 | 95 | SEQ ID NO: 9262 | 0.00% | 13.00344192 |
| 11 | 94 | SEQ ID NO: 9263 | 0.00% | 10.01276388 |
| 12 | 99 | SEQ ID NO: 9264 | 0.00% | 5.6615328 |
| 13 | 39 | SEQ ID NO: 9265 | 0.00% | 3.6304212 |
| 14 | 111 | SEQ ID NO: 9266 | 0.00% | 2.53368 |
| 15 | 103 | SEQ ID NO: 9267 | 0.00% | 2.475394803 |
| 16 | 14 | SEQ ID NO: 9268 | 0.00% | 2.4519012 |
| 17 | 19 | SEQ ID NO: 9269 | 0.00% | 2.07604992 |
| 18 | 29 | SEQ ID NO: 9270 | 0.00% | 1.8179154 |
| 19 | 57 | SEQ ID NO: 9271 | 0.00% | 1.52076 |
| 20 | 47 | SEQ ID NO: 9272 | 0.00% | 1.27712376 |

HLA A 1101 - 9 mers

| Maximum possible score using this molecule type | | | | 36 |
|---|---|---|---|---|
| 1 | 80 | SEQ ID NO: 9273 | 3.33% | 1.2 |
| 2 | 69 | SEQ ID NO: 9274 | 1.66% | 0.6 |
| 3 | 109 | SEQ ID NO: 9275 | 1.66% | 0.6 |

HLA A 1101 - 10 mers

| Maximum possible score using this molecule type | | | | 36 |
|---|---|---|---|---|
| 1 | 22 | SEQ ID NO: 9276 | 11.11% | 4 |

HLA B7 - 9 mers

| Maximum possible score using this molecule type | | | | 5400 |
|---|---|---|---|---|
| 1 | 22 | SEQ ID NO: 9277 | 3.70% | 200 |
| 2 | 77 | SEQ ID NO: 9278 | 2.22% | 120 |
| 3 | 104 | SEQ ID NO: 9279 | 0.22% | 12 |
| 4 | 40 | SEQ ID NO: 9280 | 0.11% | 6 |
| 5 | 8 | SEQ ID NO: 9281 | 0.07% | 4 |
| 6 | 21 | SEQ ID NO: 9282 | 0.07% | 4 |
| 7 | 68 | SEQ ID NO: 9283 | 0.07% | 4 |
| 8 | 92 | SEQ ID NO: 9284 | 0.07% | 4 |
| 9 | 102 | SEQ ID NO: 9285 | 0.07% | 4 |
| 10 | 46 | SEQ ID NO: 9286 | 0.03% | 2 |
| 11 | 98 | SEQ ID NO: 9287 | 0.03% | 2 |
| 12 | 103 | SEQ ID NO: 9288 | 0.03% | 2 |
| 13 | 88 | SEQ ID NO: 9289 | 0.02% | 1.2 |
| 14 | 105 | SEQ ID NO: 9290 | 0.01% | 0.9 |
| 15 | 43 | SEQ ID NO: 9291 | 0.01% | 0.6 |
| 16 | 79 | SEQ ID NO: 9292 | 0.01% | 0.6 |
| 17 | 95 | SEQ ID NO: 9293 | 0.01% | 0.6 |
| 18 | 107 | SEQ ID NO: 9294 | 0.00% | 0.5 |

HLA B7 - 10 mers

| Maximum possible score using this molecule type | | | | 5400 |
|---|---|---|---|---|
| 1 | 46 | SEQ ID NO: 9295 | 1.48% | 80 |
| 2 | 98 | SEQ ID NO: 9296 | 1.48% | 80 |
| 3 | 91 | SEQ ID NO: 9297 | 0.37% | 20 |
| 4 | 103 | SEQ ID NO: 9298 | 0.37% | 20 |
| 5 | 7 | SEQ ID NO: 9299 | 0.07% | 4 |
| 6 | 21 | SEQ ID NO: 9300 | 0.07% | 4 |
| 7 | 101 | SEQ ID NO: 9301 | 0.07% | 4 |
| 8 | 107 | SEQ ID NO: 9302 | 0.03% | 2 |
| 9 | 67 | SEQ ID NO: 9303 | 0.02% | 1.2 |
| 10 | 93 | SEQ ID NO: 9304 | 0.02% | 1.2 |
| 11 | 69 | SEQ ID NO: 9305 | 0.01% | 1 |
| 12 | 39 | SEQ ID NO: 9306 | 0.01% | 0.6 |
| 13 | 77 | SEQ ID NO: 9307 | 0.01% | 0.6 |
| 14 | 22 | SEQ ID NO: 9308 | 0.00% | 0.5 |

TABLE 23
Epitopes for SEQ ID NO: 6049

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 0 | SEQ ID NO: 9309 | 0.2% | 11.25 |
| 2 | 35 | SEQ ID NO: 9310 | 0.01% | 0.9 |
| 3 | 4 | SEQ ID NO: 9311 | 0.00% | 0.5 |
| 4 | 5 | SEQ ID NO: 9312 | 0.00% | 0.5 |
| 5 | 10 | SEQ ID NO: 9313 | 0.00% | 0.5 |
| 6 | 19 | SEQ ID NO: 9314 | 0.00% | 0.5 |
| 7 | 21 | SEQ ID NO: 9315 | 0.00% | 0.5 |

HLA A1 - 10 mers

| Maximum possible score using this molecule type | | | | 5625 |
|---|---|---|---|---|
| 1 | 0 | SEQ ID NO: 9316 | 0.2% | 11.25 |
| 2 | 5 | SEQ ID NO: 9317 | 0.04% | 2.5 |
| 3 | 33 | SEQ ID NO: 9318 | 0.02% | 1.5 |
| 4 | 3 | SEQ ID NO: 9319 | 0.02% | 1.25 |
| 5 | 9 | SEQ ID NO: 9320 | 0.00% | 0.5 |
| 6 | 18 | SEQ ID NO: 9321 | 0.00% | 0.5 |
| 7 | 20 | SEQ ID NO: 9322 | 0.00% | 0.5 |

HLA A3 - 9 mers

| Maximum possible score using this molecule type | | | | 12150 |
|---|---|---|---|---|
| 1 | 4 | SEQ ID NO: 9323 | 0.14% | 18 |
| 2 | 16 | SEQ ID NO: 9324 | 0.11% | 13.5 |
| 3 | 23 | SEQ ID NO: 9325 | 0.06% | 8.1 |
| 4 | 18 | SEQ ID NO: 9326 | 0.03% | 4.05 |
| 5 | 21 | SEQ ID NO: 9327 | 0.01% | 2.025 |
| 6 | 9 | SEQ ID NO: 9328 | 0.01% | 1.8 |
| 7 | 15 | SEQ ID NO: 9329 | 0.01% | 1.8 |
| 8 | 25 | SEQ ID NO: 9330 | 0.01% | 1.8 |
| 9 | 12 | SEQ ID NO: 9331 | 0.00% | 0.9 |
| 10 | 19 | SEQ ID NO: 9332 | 0.00% | 0.9 |
| 11 | 20 | SEQ ID NO: 9333 | 0.00% | 0.9 |
| 12 | 2 | SEQ ID NO: 9334 | 0.00% | 0.81 |
| 13 | 22 | SEQ ID NO: 9335 | 0.00% | 0.81 |
| 14 | 10 | SEQ ID NO: 9336 | 0.00% | 0.6 |

HLA A3 - 10 mers

| Maximum possible score using this molecule type | | | | 12150 |
|---|---|---|---|---|
| 1 | 20 | SEQ ID NO: 9337 | 0.16% | 20.25 |
| 2 | 9 | SEQ ID NO: 9338 | 0.09% | 12 |
| 3 | 16 | SEQ ID NO: 9339 | 0.07% | 9 |
| 4 | 18 | SEQ ID NO: 9340 | 0.07% | 9 |
| 5 | 22 | SEQ ID NO: 9341 | 0.06% | 8.1 |
| 6 | 4 | SEQ ID NO: 9342 | 0.03% | 4.05 |
| 7 | 15 | SEQ ID NO: 9343 | 0.03% | 4.05 |
| 8 | 12 | SEQ ID NO: 9344 | 0.02% | 3.6 |
| 9 | 3 | SEQ ID NO: 9345 | 0.00% | 0.9 |
| 10 | 33 | SEQ ID NO: 9346 | 0.00% | 0.6 |
| 11 | 2 | SEQ ID NO: 9347 | 0.00% | 0.54 |
| 12 | 24 | SEQ ID NO: 9348 | 0.00% | 0.54 |

HLA A24 - 9 mers

| Maximum possible score using this molecule type | | | | 1596.672 |
|---|---|---|---|---|
| 1 | 8 | SEQ ID NO: 9349 | 18.78% | 300 |
| 2 | 11 | SEQ ID NO: 9350 | 1.87% | 30 |
| 3 | 28 | SEQ ID NO: 9351 | 1.50% | 24 |
| 4 | 7 | SEQ ID NO: 9352 | 0.75% | 12 |
| 5 | 17 | SEQ ID NO: 9353 | 0.56% | 9 |
| 6 | 14 | SEQ ID NO: 9354 | 0.46% | 7.5 |
| 7 | 23 | SEQ ID NO: 9355 | 0.37% | 6 |
| 8 | 13 | SEQ ID NO: 9356 | 0.36% | 5.76 |
| 9 | 2 | SEQ ID NO: 9357 | 0.35% | 5.6 |
| 10 | 16 | SEQ ID NO: 9358 | 0.35% | 5.6 |
| 11 | 9 | SEQ ID NO: 9359 | 0.30% | 4.8 |
| 12 | 21 | SEQ ID NO: 9360 | 0.26% | 4.2 |
| 13 | 5 | SEQ ID NO: 9361 | 0.25% | 4 |
| 14 | 4 | SEQ ID NO: 9362 | 0.22% | 3.6 |
| 15 | 0 | SEQ ID NO: 9363 | 0.18% | 3 |
| 16 | 19 | SEQ ID NO: 9364 | 0.18% | 3 |
| 17 | 10 | SEQ ID NO: 9365 | 0.15% | 2.4 |
| 18 | 18 | SEQ ID NO: 9366 | 0.13% | 2.1 |
| 19 | 25 | SEQ ID NO: 9367 | 0.06% | 1.1 |
| 20 | 15 | SEQ ID NO: 9368 | 0.05% | 0.9 |

HLA A24 - 10 mers

| Maximum possible score using this molecule type | | | | 1596.672 |
|---|---|---|---|---|
| 1 | 8 | SEQ ID NO: 9369 | 22.54% | 360 |
| 2 | 7 | SEQ ID NO: 9370 | 1.25% | 20 |
| 3 | 17 | SEQ ID NO: 9371 | 0.65% | 10.5 |
| 4 | 15 | SEQ ID NO: 9372 | 0.52% | 8.4 |
| 5 | 4 | SEQ ID NO: 9373 | 0.45% | 7.2 |
| 6 | 22 | SEQ ID NO: 9374 | 0.37% | 6 |
| 7 | 12 | SEQ ID NO: 9375 | 0.36% | 5.76 |
| 8 | 27 | SEQ ID NO: 9376 | 0.30% | 4.8 |
| 9 | 14 | SEQ ID NO: 9377 | 0.28% | 4.5 |
| 10 | 20 | SEQ ID NO: 9378 | 0.26% | 4.2 |
| 11 | 10 | SEQ ID NO: 9379 | 0.25% | 4 |
| 12 | 3 | SEQ ID NO: 9380 | 0.18% | 3 |
| 13 | 18 | SEQ ID NO: 9381 | 0.18% | 3 |
| 14 | 9 | SEQ ID NO: 9382 | 0.15% | 2.4 |
| 15 | 24 | SEQ ID NO: 9383 | 0.10% | 1.65 |
| 16 | 16 | SEQ ID NO: 9384 | 0.07% | 1.2 |
| 17 | 13 | SEQ ID NO: 9385 | 0.06% | 1 |
| 18 | 11 | SEQ ID NO: 9386 | 0.05% | 0.9 |
| 19 | 1 | SEQ ID NO: 9387 | 0.05% | 0.84 |

HLA A 0201 - 9 mers

| Maximum possible score using this molecule type | | | | 3925227.1 |
|---|---|---|---|---|
| 1 | 12 | SEQ ID NO: 9388 | 0.10% | 4267.988928 |
| 2 | 23 | SEQ ID NO: 9389 | 0.03% | 1360.69088544 |
| 3 | 9 | SEQ ID NO: 9390 | 0.01% | 569.948832 |
| 4 | 16 | SEQ ID NO: 9391 | 0.00% | 309.0498408 |
| 5 | 15 | SEQ ID NO: 9392 | 0.00% | 79.73570448 |
| 6 | 2 | SEQ ID NO: 9393 | 0.00% | 51.109542 |
| 7 | 18 | SEQ ID NO: 9394 | 0.00% | 45.25539984 |
| 8 | 25 | SEQ ID NO: 9395 | 0.00% | 34.28765802 |
| 9 | 22 | SEQ ID NO: 9396 | 0.00% | 26.532116325 |
| 10 | 5 | SEQ ID NO: 9397 | 0.00% | 25.26691266 |
| 11 | 21 | SEQ ID NO: 9398 | 0.00% | 4.72873208445 |
| 12 | 11 | SEQ ID NO: 9399 | 0.00% | 2.638538265 |
| 13 | 8 | SEQ ID NO: 9400 | 0.00% | 2.4274552038 |
| 14 | 4 | SEQ ID NO: 9401 | 0.00% | 1.7415324 |
| 15 | 20 | SEQ ID NO: 9402 | 0.00% | 1.6025526 |
| 16 | 13 | SEQ ID NO: 9403 | 0.00% | 1.453803297 |
| 17 | 35 | SEQ ID NO: 9404 | 0.00% | 1.36878336 |
| 18 | 3 | SEQ ID NO: 9405 | 0.00% | 0.824619 |
| 19 | 33 | SEQ ID NO: 9406 | 0.00% | 0.513774 |

HLA A 0201 - 10 mers

| Maximum possible score using this molecule type | | | | 3925227.1 |
|---|---|---|---|---|
| 1 | 22 | SEQ ID NO: 9407 | 0.09% | 3636.068421648 |
| 2 | 4 | SEQ ID NO: 9408 | 0.02% | 1107.960876 |
| 3 | 15 | SEQ ID NO: 9409 | 0.02% | 836.2525104 |
| 4 | 16 | SEQ ID NO: 9410 | 0.00% | 150.9313176 |
| 5 | 12 | SEQ ID NO: 9411 | 0.00% | 76.55002416 |
| 6 | 1 | SEQ ID NO: 9412 | 0.00% | 49.0273014 |
| 7 | 10 | SEQ ID NO: 9413 | 0.00% | 42.1638414747 |
| 8 | 20 | SEQ ID NO: 9414 | 0.00% | 9.29480508 |
| 9 | 24 | SEQ ID NO: 9415 | 0.00% | 9.2669346 |
| 10 | 13 | SEQ ID NO: 9416 | 0.00% | 7.96581954 |
| 11 | 21 | SEQ ID NO: 9417 | 0.00% | 5.051306761875 |
| 12 | 5 | SEQ ID NO: 9418 | 0.00% | 2.6941464 |
| 13 | 11 | SEQ ID NO: 9419 | 0.00% | 2.3839914 |
| 14 | 34 | SEQ ID NO: 9420 | 0.00% | 1.465422 |
| 15 | 2 | SEQ ID NO: 9421 | 0.00% | 0.70794 |
| 16 | 9 | SEQ ID NO: 9422 | 0.00% | 0.6513048 |
| 17 | 19 | SEQ ID NO: 9423 | 0.00% | 0.51882640425 |

HLA A 1101 - 10 mers

| | |
|---|---|
| Maximum possible score using this molecule type | 36 |

HLA A 1101 - 10 mers

| Maximum possible score using this molecule type | | | | 36 |
|---|---|---|---|---|
| 1 | 33 | SEQ ID NO: 9424 | 1.66% | 0.6 |

HLA B7 - 9 mers

| Maximum possible score using this molecule type | | | | 5400 |
|---|---|---|---|---|
| 1 | 13 | SEQ ID NO: 9425 | 0.22% | 12 |
| 2 | 2 | SEQ ID NO: 9426 | 0.07% | 4 |
| 3 | 9 | SEQ ID NO: 9427 | 0.07% | 4 |
| 4 | 16 | SEQ ID NO: 9428 | 0.07% | 4 |
| 5 | 23 | SEQ ID NO: 9429 | 0.07% | 4 |
| 6 | 5 | SEQ ID NO: 9430 | 0.02% | 1.2 |
| 7 | 15 | SEQ ID NO: 9431 | 0.01% | 1 |

HLA B7 - 10 mers

| Maximum possible score using this molecule type | | | | 5400 |
|---|---|---|---|---|
| 1 | 4 | SEQ ID NO: 9432 | 0.07% | 4 |
| 2 | 10 | SEQ ID NO: 9433 | 0.07% | 4 |
| 3 | 12 | SEQ ID NO: 9434 | 0.07% | 4 |
| 4 | 15 | SEQ ID NO: 9435 | 0.07% | 4 |
| 5 | 22 | SEQ ID NO: 9436 | 0.07% | 4 |
| 6 | 13 | SEQ ID NO: 9437 | 0.02% | 1.2 |

TABLE 24
Epitopes for SEQ ID NO: 6050

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 47 | SEQ ID NO: 9438 | 0.01% | 0.75 |
| 2 | 21 | SEQ ID NO: 9439 | 0.00% | 0.5 |
| 3 | 53 | SEQ ID NO: 9440 | 0.00% | 0.5 |

HLA A1 - 10 mers

| Maximum possible score using this molecule type | | | | 5625 |
|---|---|---|---|---|
| 1 | 16 | SEQ ID NO: 9441 | 0.04% | 2.5 |
| 2 | 71 | SEQ ID NO: 9442 | 0.04% | 2.5 |
| 3 | 47 | SEQ ID NO: 9443 | 0.02% | 1.5 |
| 4 | 62 | SEQ ID NO: 9444 | 0.01% | 0.9 |
| 5 | 20 | SEQ ID NO: 9445 | 0.00% | 0.5 |
| 6 | 38 | SEQ ID NO: 9446 | 0.00% | 0.5 |

HLA A3 - 9 mers

| Maximum possible score using this molecule type | | | | 12150 |
|---|---|---|---|---|
| 1 | 54 | SEQ ID NO: 9447 | 0.02% | 2.7 |
| 2 | 17 | SEQ ID NO: 9448 | 0.01% | 2 |
| 3 | 3 | SEQ ID NO: 9449 | 0.01% | 1.8 |

HLA A3 - 10 mers

| Maximum possible score using this molecule type | | | | 12150 |
|---|---|---|---|---|
| 1 | 22 | SEQ ID NO: 9450 | 0.09% | 12 |
| 2 | 16 | SEQ ID NO: 9451 | 0.01% | 2 |
| 3 | 54 | SEQ ID NO: 9452 | 0.00% | 0.9 |

HLA A24 - 9 mers

| Maximum possible score using this molecule type | | | | 1596.672 |
|---|---|---|---|---|
| 1 | 70 | SEQ ID NO: 9453 | 2.10% | 33.6 |
| 2 | 7 | SEQ ID NO: 9454 | 1.12% | 18 |
| 3 | 60 | SEQ ID NO: 9455 | 0.46% | 7.5 |
| 4 | 54 | SEQ ID NO: 9456 | 0.37% | 6 |
| 5 | 14 | SEQ ID NO: 9457 | 0.31% | 5 |
| 6 | 19 | SEQ ID NO: 9458 | 0.30% | 4.8 |
| 7 | 47 | SEQ ID NO: 9459 | 0.30% | 4.8 |
| 8 | 12 | SEQ ID NO: 9460 | 0.25% | 4 |
| 9 | 15 | SEQ ID NO: 9461 | 0.25% | 4 |
| 10 | 67 | SEQ ID NO: 9462 | 0.25% | 4 |
| 11 | 21 | SEQ ID NO: 9463 | 0.18% | 3 |
| 12 | 37 | SEQ ID NO: 9464 | 0.06% | 1 |
| 13 | 27 | SEQ ID NO: 9465 | 0.03% | 0.5 |

HLA A24 - 10 mers

| Maximum possible score using this molecule type | | | | 1596.672 |
|---|---|---|---|---|
| 1 | 14 | SEQ ID NO: 9466 | 12.52% | 200 |
| 2 | 7 | SEQ ID NO: 9467 | 0.93% | 15 |
| 3 | 11 | SEQ ID NO: 9468 | 0.75% | 12 |
| 4 | 60 | SEQ ID NO: 9469 | 0.56% | 9 |
| 5 | 18 | SEQ ID NO: 9470 | 0.45% | 7.2 |
| 6 | 46 | SEQ ID NO: 9471 | 0.45% | 7.2 |
| 7 | 53 | SEQ ID NO: 9472 | 0.37% | 6 |
| 8 | 69 | SEQ ID NO: 9473 | 0.35% | 5.6 |
| 9 | 66 | SEQ ID NO: 9474 | 0.25% | 4 |
| 10 | 20 | SEQ ID NO: 9475 | 0.12% | 2 |
| 11 | 47 | SEQ ID NO: 9476 | 0.07% | 1.2 |
| 12 | 36 | SEQ ID NO: 9477 | 0.06% | 1 |
| 13 | 26 | SEQ ID NO: 9478 | 0.04% | 0.75 |
| 14 | 70 | SEQ ID NO: 9479 | 0.04% | 0.72 |

HLA A 0201 - 9 mers

| Maximum possible score using this molecule type | | | | 3925227.1 |
|---|---|---|---|---|
| 1 | 54 | SEQ ID NO: 9480 | 0.02% | 881.199 |
| 2 | 26 | SEQ ID NO: 9481 | 0.00% | 95.013 |
| 3 | 61 | SEQ ID NO: 9482 | 0.00% | 93.69648 |
| 4 | 19 | SEQ ID NO: 9483 | 0.00% | 40.2894864 |
| 5 | 74 | SEQ ID NO: 9484 | 0.00% | 12.6684 |
| 6 | 35 | SEQ ID NO: 9485 | 0.00% | 10.34586 |
| 7 | 69 | SEQ ID NO: 9486 | 0.00% | 3.3704706 |
| 8 | 13 | SEQ ID NO: 9487 | 0.00% | 1.656 |
| 9 | 15 | SEQ ID NO: 9488 | 0.00% | 1.47537042 |
| 10 | 68 | SEQ ID NO: 9489 | 0.00% | 0.966 |
| 11 | 22 | SEQ ID NO: 9490 | 0.00% | 0.942678 |
| 12 | 12 | SEQ ID NO: 9491 | 0.00% | 0.7669695 |
| 13 | 36 | SEQ ID NO: 9492 | 0.00% | 0.52661835 |

HLA A 0201 - 10 mers

| Maximum possible score using this molecule type | | | | 3925227.1 |
|---|---|---|---|---|
| 1 | 61 | SEQ ID NO: 9493 | 0.00% | 93.69648 |
| 2 | 25 | SEQ ID NO: 9494 | 0.00% | 63.33035625 |
| 3 | 34 | SEQ ID NO: 9495 | 0.00% | 50.232 |
| 4 | 53 | SEQ ID NO: 9496 | 0.00% | 45.2838375 |
| 5 | 26 | SEQ ID NO: 9497 | 0.00% | 14.35752 |
| 6 | 27 | SEQ ID NO: 9498 | 0.00% | 2.8557858 |
| 7 | 17 | SEQ ID NO: 9499 | 0.00% | 2.3973222 |
| 8 | 36 | SEQ ID NO: 9500 | 0.00% | 1.798209 |
| 9 | 69 | SEQ ID NO: 9501 | 0.00% | 1.03521597 |
| 10 | 67 | SEQ ID NO: 9502 | 0.00% | 0.966 |
| 11 | 68 | SEQ ID NO: 9503 | 0.00% | 0.910938 |
| 12 | 11 | SEQ ID NO: 9504 | 0.00% | 0.7669695 |

HLA A 1101 - 9 mers

| Maximum possible score using this molecule type | | | | 36 |
|---|---|---|---|---|
| 1 | 17 | SEQ ID NO: 9505 | 2.22% | 0.8 |

HLA A 1101 - 10 mers

| Maximum possible score using this molecule type | | | | 36 |
|---|---|---|---|---|
| 1 | 16 | SEQ ID NO: 9506 | 5.55% | 2 |

HLA B7 - 9 mers

| Maximum possible score using this molecule type | | | | 5400 |
|---|---|---|---|---|
| 1 | 27 | SEQ ID NO: 9507 | 0.37% | 20 |
| 2 | 54 | SEQ ID NO: 9508 | 0.22% | 12 |
| 3 | 70 | SEQ ID NO: 9509 | 0.22% | 12 |
| 4 | 67 | SEQ ID NO: 9510 | 0.11% | 6 |
| 5 | 12 | SEQ ID NO: 9511 | 0.07% | 4 |
| 6 | 15 | SEQ ID NO: 9512 | 0.07% | 4 |
| 7 | 19 | SEQ ID NO: 9513 | 0.07% | 4 |
| 8 | 49 | SEQ ID NO: 9514 | 0.03% | 2 |
| 9 | 69 | SEQ ID NO: 9515 | 0.03% | 1.8 |
| 10 | 47 | SEQ ID NO: 9516 | 0.02% | 1.2 |
| 11 | 5 | SEQ ID NO: 9517 | 0.01% | 1 |
| 12 | 9 | SEQ ID NO: 9518 | 0.01% | 1 |
| 13 | 35 | SEQ ID NO: 9519 | 0.01% | 1 |
| 14 | 37 | SEQ ID NO: 9520 | 0.01% | 0.6 |
| 15 | 68 | SEQ ID NO: 9521 | 0.01% | 0.6 |

HLA B7 - 10 mers

| Maximum possible score using this molecule type | | | | 5400 |
|---|---|---|---|---|
| 1 | 69 | SEQ ID NO: 9522 | 0.66% | 36 |
| 2 | 53 | SEQ ID NO: 9523 | 0.22% | 12 |
| 3 | 5 | SEQ ID NO: 9524 | 0.13% | 7.5 |
| 4 | 66 | SEQ ID NO: 9525 | 0.11% | 6 |
| 5 | 11 | SEQ ID NO: 9526 | 0.07% | 4 |
| 6 | 27 | SEQ ID NO: 9527 | 0.07% | 4 |
| 7 | 46 | SEQ ID NO: 9528 | 0.07% | 4 |
| 8 | 18 | SEQ ID NO: 9529 | 0.02% | 1.2 |
| 9 | 9 | SEQ ID NO: 9530 | 0.01% | 1 |
| 10 | 26 | SEQ ID NO: 9531 | 0.01% | 1 |
| 11 | 25 | SEQ ID NO: 9532 | 0.01% | 0.75 |
| 12 | 17 | SEQ ID NO: 9533 | 0.01% | 0.6 |
| 13 | 36 | SEQ ID NO: 9534 | 0.01% | 0.6 |
| 14 | 68 | SEQ ID NO: 9535 | 0.01% | 0.6 |
| 15 | 35 | SEQ ID NO: 9536 | 0.00% | 0.5 |
| 16 | 42 | SEQ ID NO: 9537 | 0.00% | 0.5 |
| 17 | 73 | SEQ ID NO: 9538 | 0.00% | 0.5 |

TABLE 25
Epitopes for SEQ ID NO: 6052

| | | | | |
|---|---|---|---|---|
| | Start | | % of max. | |
| Rank | position | Sequence | score | Score |

HLA A1 - 9 mers

| Maximum possible score using this molecule type | | | | 5625 |
|---|---|---|---|---|
| 1 | 365 | SEQ ID NO: 9539 | 0.8% | 45 |
| 2 | 397 | SEQ ID NO: 9540 | 0.44% | 25 |
| 3 | 229 | SEQ ID NO: 9541 | 0.32% | 18 |
| 4 | 103 | SEQ ID NO: 9542 | 0.17% | 10 |
| 5 | 338 | SEQ ID NO: 9543 | 0.17% | 10 |
| 6 | 251 | SEQ ID NO: 9544 | 0.16% | 9 |
| 7 | 79 | SEQ ID NO: 9545 | 0.11% | 6.25 |
| 8 | 119 | SEQ ID NO: 9546 | 0.10% | 6 |
| 9 | 361 | SEQ ID NO: 9547 | 0.08% | 5 |
| 10 | 60 | SEQ ID NO: 9548 | 0.04% | 2.25 |
| 11 | 101 | SEQ ID NO: 9549 | 0.04% | 2.25 |
| 12 | 278 | SEQ ID NO: 9550 | 0.04% | 2.25 |
| 13 | 23 | SEQ ID NO: 9551 | 0.02% | 1.25 |
| 14 | 164 | SEQ ID NO: 9552 | 0.02% | 1.25 |
| 15 | 165 | SEQ ID NO: 9553 | 0.02% | 1.25 |
| 16 | 295 | SEQ ID NO: 9554 | 0.02% | 1.25 |
| 17 | 172 | SEQ ID NO: 9555 | 0.01% | 0.9 |
| 18 | 0 | SEQ ID NO: 9556 | 0.01% | 0.75 |
| 19 | 311 | SEQ ID NO: 9557 | 0.01% | 0.75 |
| 20 | 78 | SEQ ID NO: 9558 | 0.01% | 0.625 |

HLA A1 - 10 mers

| Maximum possible score using this molecule type | | | | 5625 |
|---|---|---|---|---|
| 1 | 114 | SEQ ID NO: 9559 | 1.11% | 62.5 |
| 2 | 134 | SEQ ID NO: 9560 | 0.8% | 45 |
| 3 | 365 | SEQ ID NO: 9561 | 0.8% | 45 |
| 4 | 77 | SEQ ID NO: 9562 | 0.66% | 37.5 |
| 5 | 103 | SEQ ID NO: 9563 | 0.44% | 25 |
| 6 | 23 | SEQ ID NO: 9564 | 0.22% | 12.5 |
| 7 | 338 | SEQ ID NO: 9565 | 0.17% | 10 |
| 8 | 361 | SEQ ID NO: 9566 | 0.17% | 10 |
| 9 | 324 | SEQ ID NO: 9567 | 0.11% | 6.25 |
| 10 | 375 | SEQ ID NO: 9568 | 0.11% | 6.25 |
| 11 | 79 | SEQ ID NO: 9569 | 0.04% | 2.5 |
| 12 | 295 | SEQ ID NO: 9570 | 0.04% | 2.5 |
| 13 | 346 | SEQ ID NO: 9571 | 0.04% | 2.5 |
| 14 | 378 | SEQ ID NO: 9572 | 0.03% | 2 |
| 15 | 251 | SEQ ID NO: 9573 | 0.03% | 1.8 |
| 16 | 214 | SEQ ID NO: 9574 | 0.02% | 1.125 |
| 17 | 160 | SEQ ID NO: 9575 | 0.01% | 1 |
| 18 | 172 | SEQ ID NO: 9576 | 0.01% | 0.9 |
| 19 | 229 | SEQ ID NO: 9577 | 0.01% | 0.9 |
| 20 | 376 | SEQ ID NO: 9578 | 0.01% | 0.9 |

HLA A3 - 9 mers

| Maximum possible score using this molecule type | | | | 12150 |
|---|---|---|---|---|
| 1 | 229 | SEQ ID NO: 9579 | 0.49% | 60 |
| 2 | 361 | SEQ ID NO: 9580 | 0.27% | 33.75 |
| 3 | 330 | SEQ ID NO: 9581 | 0.16% | 20 |
| 4 | 218 | SEQ ID NO: 9582 | 0.09% | 12 |
| 5 | 338 | SEQ ID NO: 9583 | 0.04% | 6 |
| 6 | 352 | SEQ ID NO: 9584 | 0.04% | 6 |
| 7 | 103 | SEQ ID NO: 9585 | 0.04% | 5.4 |
| 8 | 291 | SEQ ID NO: 9586 | 0.01% | 2 |
| 9 | 241 | SEQ ID NO: 9587 | 0.01% | 1.8 |
| 10 | 290 | SEQ ID NO: 9588 | 0.01% | 1.8 |
| 11 | 316 | SEQ ID NO: 9589 | 0.01% | 1.8 |
| 12 | 222 | SEQ ID NO: 9590 | 0.01% | 1.35 |
| 13 | 266 | SEQ ID NO: 9591 | 0.01% | 1.35 |
| 14 | 53 | SEQ ID NO: 9592 | 0.00% | 1 |
| 15 | 100 | SEQ ID NO: 9593 | 0.00% | 0.9 |
| 16 | 138 | SEQ ID NO: 9594 | 0.00% | 0.9 |
| 17 | 240 | SEQ ID NO: 9595 | 0.00% | 0.9 |
| 18 | 119 | SEQ ID NO: 9596 | 0.00% | 0.675 |
| 19 | 44 | SEQ ID NO: 9597 | 0.00% | 0.6 |
| 20 | 161 | SEQ ID NO: 9598 | 0.00% | 0.6 |

HLA A3 - 10 mers

| Maximum possible score using this molecule type | | | | 12150 |
|---|---|---|---|---|
| 1 | 338 | SEQ ID NO: 9599 | 0.49% | 60 |
| 2 | 160 | SEQ ID NO: 9600 | 0.32% | 40 |
| 3 | 352 | SEQ ID NO: 9601 | 0.24% | 30 |
| 4 | 361 | SEQ ID NO: 9602 | 0.18% | 22.5 |
| 5 | 103 | SEQ ID NO: 9603 | 0.13% | 16.2 |
| 6 | 290 | SEQ ID NO: 9604 | 0.07% | 9 |
| 7 | 351 | SEQ ID NO: 9605 | 0.07% | 9 |
| 8 | 44 | SEQ ID NO: 9606 | 0.04% | 6 |
| 9 | 228 | SEQ ID NO: 9607 | 0.03% | 4.05 |
| 10 | 394 | SEQ ID NO: 9608 | 0.02% | 3 |
| 11 | 240 | SEQ ID NO: 9609 | 0.02% | 2.7 |
| 12 | 100 | SEQ ID NO: 9610 | 0.01% | 1.8 |
| 13 | 114 | SEQ ID NO: 9611 | 0.01% | 1.8 |
| 14 | 93 | SEQ ID NO: 9612 | 0.01% | 1.5 |
| 15 | 134 | SEQ ID NO: 9613 | 0.01% | 1.5 |
| 16 | 221 | SEQ ID NO: 9614 | 0.01% | 1.35 |
| 17 | 330 | SEQ ID NO: 9615 | 0.00% | 1.2 |
| 18 | 112 | SEQ ID NO: 9616 | 0.00% | 0.9 |
| 19 | 218 | SEQ ID NO: 9617 | 0.00% | 0.9 |
| 20 | 55 | SEQ ID NO: 9618 | 0.00% | 0.6 |

HLA A24 - 9 mers

| Maximum possible score using this molecule type | | | | 1596.672 |
|---|---|---|---|---|
| 1 | 345 | SEQ ID NO: 9619 | 1.50% | 24 |
| 2 | 306 | SEQ ID NO: 9620 | 0.75% | 12 |
| 3 | 222 | SEQ ID NO: 9621 | 0.54% | 8.64 |
| 4 | 111 | SEQ ID NO: 9622 | 0.51% | 8.25 |
| 5 | 159 | SEQ ID NO: 9623 | 0.45% | 7.2 |
| 6 | 219 | SEQ ID NO: 9624 | 0.45% | 7.2 |
| 7 | 283 | SEQ ID NO: 9625 | 0.45% | 7.2 |
| 8 | 266 | SEQ ID NO: 9626 | 0.42% | 6.72 |
| 9 | 56 | SEQ ID NO: 9627 | 0.41% | 6.6 |
| 10 | 131 | SEQ ID NO: 9628 | 0.37% | 6 |
| 11 | 214 | SEQ ID NO: 9629 | 0.37% | 6 |
| 12 | 297 | SEQ ID NO: 9630 | 0.37% | 6 |
| 13 | 86 | SEQ ID NO: 9631 | 0.31% | 5 |
| 14 | 122 | SEQ ID NO: 9632 | 0.31% | 5 |
| 15 | 48 | SEQ ID NO: 9633 | 0.30% | 4.8 |
| 16 | 105 | SEQ ID NO: 9634 | 0.30% | 4.8 |
| 17 | 213 | SEQ ID NO: 9635 | 0.30% | 4.8 |
| 18 | 323 | SEQ ID NO: 9636 | 0.30% | 4.8 |
| 19 | 338 | SEQ ID NO: 9637 | 0.30% | 4.8 |
| 20 | 399 | SEQ ID NO: 9638 | 0.30% | 4.8 |

HLA A24 - 10 mers

| Maximum possible score using this molecule type | | | | 1596.672 |
|---|---|---|---|---|
| 1 | 65 | SEQ ID NO: 9639 | 0.93% | 15 |
| 2 | 306 | SEQ ID NO: 9640 | 0.75% | 12 |
| 3 | 95 | SEQ ID NO: 9641 | 0.66% | 10.56 |
| 4 | 36 | SEQ ID NO: 9642 | 0.60% | 9.6 |
| 5 | 385 | SEQ ID NO: 9643 | 0.50% | 8 |
| 6 | 111 | SEQ ID NO: 9644 | 0.46% | 7.5 |
| 7 | 104 | SEQ ID NO: 9645 | 0.45% | 7.2 |
| 8 | 214 | SEQ ID NO: 9646 | 0.45% | 7.2 |
| 9 | 221 | SEQ ID NO: 9647 | 0.45% | 7.2 |
| 10 | 277 | SEQ ID NO: 9648 | 0.45% | 7.2 |
| 11 | 150 | SEQ ID NO: 9649 | 0.37% | 6 |
| 12 | 152 | SEQ ID NO: 9650 | 0.37% | 6 |
| 13 | 158 | SEQ ID NO: 9651 | 0.37% | 6 |
| 14 | 171 | SEQ ID NO: 9652 | 0.37% | 6 |
| 15 | 343 | SEQ ID NO: 9653 | 037% | 6 |
| 16 | 110 | SEQ ID NO: 9654 | 0.34% | 5.5 |
| 17 | 85 | SEQ ID NO: 9655 | 0.31% | 5 |
| 18 | 47 | SEQ ID NO: 9656 | 0.30% | 4.8 |
| 19 | 213 | SEQ ID NO: 9657 | 0.30% | 4.8 |
| 20 | 218 | SEQ ID NO: 9658 | 0.30% | 4.8 |

HLA A 0201 - 9 mers

| Maximum possible score using this molecule type | | | | 3925227.1 |
|---|---|---|---|---|
| 1 | 222 | SEQ ID NO: 9659 | 0.03% | 1267.10434728 |
| 2 | 226 | SEQ ID NO: 9660 | 0.00% | 69.552 |
| 3 | 316 | SEQ ID NO: 9661 | 0.00% | 50.232 |
| 4 | 351 | SEQ ID NO: 9662 | 0.00% | 31.24872 |
| 5 | 159 | SEQ ID NO: 9663 | 0.00% | 13.6235739 |
| 6 | 406 | SEQ ID NO: 9664 | 0.00% | 11.4264 |
| 7 | 165 | SEQ ID NO: 9665 | 0.00% | 8.14407 |
| 8 | 238 | SEQ ID NO: 9666 | 0.00% | 7.0518 |
| 9 | 138 | SEQ ID NO: 9667 | 0.00% | 5.112072 |
| 10 | 130 | SEQ ID NO: 9668 | 0.00% | 3.00547233 |
| 11 | 303 | SEQ ID NO: 9669 | 0.00% | 2.59578 |
| 12 | 157 | SEQ ID NO: 9670 | 0.00% | 2.412585 |
| 13 | 219 | SEQ ID NO: 9671 | 0.00% | 2.103255861 |
| 14 | 305 | SEQ ID NO: 9672 | 0.00% | 1.86369 |
| 15 | 158 | SEQ ID NO: 9673 | 0.00% | 1.646892 |
| 16 | 331 | SEQ ID NO: 9674 | 0.00% | 1.614048 |
| 17 | 399 | SEQ ID NO: 9675 | 0.00% | 1.442246832 |
| 18 | 324 | SEQ ID NO: 9676 | 0.00% | 1.319625 |
| 19 | 312 | SEQ ID NO: 9677 | 0.00% | 1.233099 |
| 20 | 262 | SEQ ID NO: 9678 | 0.00% | 0.966 |

HLA A 0201 - 10 mers

| Maximum possible score using this molecule type | | | | 3925227.1 |
|---|---|---|---|---|
| 1 | 221 | SEQ ID NO: 9679 | 0.00% | 309.0498408 |
| 2 | 112 | SEQ ID NO: 9680 | 0.00% | 98.26704 |
| 3 | 330 | SEQ ID NO: 9681 | 0.00% | 98.26704 |
| 4 | 158 | SEQ ID NO: 9682 | 0.00% | 36.31608 |
| 5 | 218 | SEQ ID NO: 9683 | 0.00% | 24.0754248 |
| 6 | 124 | SEQ ID NO: 9684 | 0.00% | 12.2199 |
| 7 | 55 | SEQ ID NO: 9685 | 0.00% | 10.467576 |
| 8 | 315 | SEQ ID NO: 9686 | 0.00% | 7.7274204 |
| 9 | 350 | SEQ ID NO: 9687 | 0.00% | 4.296699 |
| 10 | 405 | SEQ ID NO: 9688 | 0.00% | 4.286487 |
| 11 | 388 | SEQ ID NO: 9689 | 0.00% | 4.054785 |
| 12 | 322 | SEQ ID NO: 9690 | 0.00% | 3.883803 |
| 13 | 130 | SEQ ID NO: 9691 | 0.00% | 3.428691903 |
| 14 | 45 | SEQ ID NO: 9692 | 0.00% | 3.411230625 |
| 15 | 132 | SEQ ID NO: 9693 | 0.00% | 2.99943 |
| 16 | 410 | SEQ ID NO: 9694 | 0.00% | 2.63718 |
| 17 | 316 | SEQ ID NO: 9695 | 0.00% | 2.48686074 |
| 18 | 104 | SEQ ID NO: 9696 | 0.00% | 2.477311485 |
| 19 | 164 | SEQ ID NO: 9697 | 0.00% | 2.2011 |
| 20 | 282 | SEQ ID NO: 9698 | 0.00% | 2.16591 |

HLA A 1101 - 9 mers

| Maximum possible score using this molecule type | | | | 36 |
|---|---|---|---|---|
| 1 | 361 | SEQ ID NO: 9699 | 16.66% | 6 |
| 2 | 53 | SEQ ID NO: 9700 | 2.77% | 1 |
| 3 | 240 | SEQ ID NO: 9701 | 1.66% | 0.6 |
| 4 | 241 | SEQ ID NO: 9702 | 1.66% | 0.6 |

HLA A 1101 - 10 mers

| Maximum possible score using this molecule type | | | | 36 |
|---|---|---|---|---|
| 1 | 361 | SEQ ID NO: 9703 | 16.66% | 6 |
| 2 | 93 | SEQ ID NO: 9704 | 8.33% | 3 |
| 3 | 338 | SEQ ID NO: 9705 | 3.33% | 1.2 |
| 4 | 134 | SEQ ID NO: 9706 | 2.77% | 1 |
| 5 | 228 | SEQ ID NO: 9707 | 2.5% | 0.9 |
| 6 | 160 | SEQ ID NO: 9708 | 2.22% | 0.8 |
| 7 | 239 | SEQ ID NO: 9709 | 1.66% | 0.6 |
| 8 | 240 | SEQ ID NO: 9710 | 1.66% | 0.6 |
| 9 | 257 | SEQ ID NO: 9711 | 1.66% | 0.6 |
| 10 | 379 | SEQ ID NO: 9712 | 1.66% | 0.6 |

HLA B7 - 9 mers

| Maximum possible score using this molecule type | | | | 5400 |
|---|---|---|---|---|
| 1 | 105 | SEQ ID NO: 9713 | 14.81% | 800 |
| 2 | 66 | SEQ ID NO: 9714 | 1.48% | 80 |
| 3 | 93 | SEQ ID NO: 9715 | 0.92% | 50 |
| 4 | 257 | SEQ ID NO: 9716 | 0.55% | 30 |
| 5 | 323 | SEQ ID NO: 9717 | 0.37% | 20 |
| 6 | 211 | SEQ ID NO: 9718 | 0.22% | 12 |
| 7 | 219 | SEQ ID NO: 9719 | 0.22% | 12 |
| 8 | 403 | SEQ ID NO: 9720 | 0.18% | 10 |
| 9 | 343 | SEQ ID NO: 9721 | 0.14% | 8 |
| 10 | 12 | SEQ ID NO: 9722 | 0.11% | 6 |
| 11 | 113 | SEQ ID NO: 9723 | 0.11% | 6 |
| 12 | 48 | SEQ ID NO: 9724 | 0.07% | 4 |
| 13 | 56 | SEQ ID NO: 9725 | 0.07% | 4 |
| 14 | 150 | SEQ ID NO: 9726 | 0.07% | 4 |
| 15 | 153 | SEQ ID NO: 9727 | 0.07% | 4 |
| 16 | 159 | SEQ ID NO: 9728 | 0.07% | 4 |
| 17 | 213 | SEQ ID NO: 9729 | 0.07% | 4 |
| 18 | 216 | SEQ ID NO: 9730 | 0.07% | 4 |
| 19 | 222 | SEQ ID NO: 9731 | 0.07% | 4 |
| 20 | 283 | SEQ ID NO: 9732 | 0.07% | 4 |

HLA B7 - 10 mers

| Maximum possible score using this molecule type | | | | 5400 |
|---|---|---|---|---|
| 1 | 36 | SEQ ID NO: 9733 | 1.48% | 80 |
| 2 | 150 | SEQ ID NO: 9734 | 1.48% | 80 |
| 3 | 343 | SEQ ID NO: 9735 | 1.48% | 80 |
| 4 | 12 | SEQ ID NO: 9736 | 1.11% | 60 |
| 5 | 308 | SEQ ID NO: 9737 | 1.11% | 60 |
| 6 | 130 | SEQ ID NO: 9738 | 0.37% | 20 |
| 7 | 55 | SEQ ID NO: 9739 | 0.22% | 12 |
| 8 | 210 | SEQ ID NO: 9740 | 0.22% | 12 |
| 9 | 218 | SEQ ID NO: 9741 | 0.22% | 12 |
| 10 | 201 | SEQ ID NO: 9742 | 0.18% | 10 |
| 11 | 121 | SEQ ID NO: 9743 | 0.14% | 8 |
| 12 | 391 | SEQ ID NO: 9744 | 0.13% | 7.5 |
| 13 | 112 | SEQ ID NO: 9745 | 0.11% | 6 |
| 14 | 385 | SEQ ID NO: 9746 | 0.11% | 6 |
| 15 | 47 | SEQ ID NO: 9747 | 0.07% | 4 |
| 16 | 66 | SEQ ID NO: 9748 | 0.07% | 4 |
| 17 | 95 | SEQ ID NO: 9749 | 0.07% | 4 |
| 18 | 104 | SEQ ID NO: 9750 | 0.07% | 4 |
| 19 | 152 | SEQ ID NO: 9751 | 0.07% | 4 |
| 20 | 158 | SEQ ID NO: 9752 | 0.07% | 4 |

TABLE 26
Cloned sequences for E. coli expression

| | |
|---|---|
| DNA length | Cloning |

| ORF | bp | pET | pGEX |
|---|---|---|---|
| P28 | 537 | NdeI/XhoI | |
| P65 | 1917 | NheI/HindIII | |
| Nsp1A | 2495 | NheI/XhoI | |
| Nsp1B | 2153 | NdeI/XhoI | |
| Nsp1C | 2612 | NdeI/XhoI | |
| Nsp2A | 431 | NdeI/XhoI | BamHI/XhoI |
| Nsp2B | 426 | NdeI/XhoI | BamHI/XhoI |
| Nsp3 | 870 | NdeI/XhoI | |
| Nsp4 | 249 | NdeI/XhoI | BamHI/XhoI |
| Nsp5 | 594 | NheI/XhoI | |
| Nsp6 | 339 | NdeI/XhoI | BamHI/XhoI |
| Nsp7 | 417 | NdeI/XhoI | BamHI/XhoI |
| Nsp9A | 1385 | NheI/XhoI | |
| Nsp9B | 1409 | NdeI/XhoI | |
| Nsp10 | 1803 | NheI/XhoI | |
| Nsp11 | 1581 | NdeI/XhoI | |
| Nsp12 | 1038 | NdeI/HindIII | |
| Nsp13 | 897 | NdeI/XhoI | |
| Spike (S1) | 1946 | NdeI/XhoI | |
| Spike (S2) | 1598 | NdeI/XhoI | |
| Spike (S1-S2) | 3545 | NdeI/XhoI | |
| HR1 | 287 | NdeI/XhoI | BamHI/XhoI |
| HR2 | 146 | NdeI/XhoI | BamHI/XhoI |
| ORF3Δ100 | 525 | NdeI/XhoI | |
| ORF4 | 465 | NdeI/XhoI | |
| Envelope (E) | 231 | NdeI/XhoI | BamHI/XhoI |
| Matrix (M)Δ100 | 366 | NdeI/XhoI | BamHI/XhoI |
| ORF7Δ18 | 137 | NdeI/XhoI | BamHI/XhoI |
| ORF8 | 369 | NdeI/XhoI | BamHI/XhoI |
| ORF9 | 135 | NdeI/XhoI | BamHI/XhoI |
| ORF10 | 120 | NheI/XhoI | BamHI/XhoI |
| ORF11 | 255 | NdeI/XhoI | BamHI/XhoI |
| Nucleocapsid (N) | 1269 | NdeI/EcoRI | |
| ORF12 | 297 | NdeI/EcoRI | BamHI/EcoRI |

TABLE 27
Primers

| ORF | Forward primer | Reverse primer |
|---|---|---|
| P28 | 9803 | 9818 |
| P65 | 9804 | 9819 |
| Nsp1A | 9805 | 9820 |
| Nsp1B | 9806 | 9821 |
| Nsp1C | 9807 | 9822 |
| Nsp2 + Nsp3 | 9808 | 9823 |
| Nsp4 to Nsp7 | 9809 | 9824 |
| Nsp9A | 9810 | 9825 |
| Nsp9B | 9811 | 9826 |
| Nsp10 | 9812 | 9827 |
| Nsp11 | 9813 | 9828 |
| Nsp12-Nsp13 | 9814 | 9829 |
| ORF3-ORF4 | 9815 | 9830 |
| Env-ORF10 | 9816 | 9831 |
| ORF11-ORF12 | 9817 | 9832 |

TABLE 28
Primers

| ORF | Forward primer | Reverse primer |
|---|---|---|
| Nsp2A | SEQ ID NO: 9833 | SEQ ID NO: 9858 |
| Nsp2B | SEQ ID NO: 9834 | SEQ ID NO: 9859 |
| Nsp3 | SEQ ID NO: 9835 | SEQ ID NO: 9860 |
| Nsp4 | SEQ ID NO: 9836 | SEQ ID NO: 9861 |
| Nsp5 | SEQ ID NO: 9837 | SEQ ID NO: 9862 |
| Nsp6 | SEQ ID NO: 9838 | SEQ ID NO: 9863 |
| Nsp7 | SEQ ID NO: 9839 | SEQ ID NO: 9864 |
| Nsp12 | SEQ ID NO: 9840 | SEQ ID NO: 9865 |
| Nsp13 | SEQ ID NO: 9841 | SEQ ID NO: 9866 |
| Spike S1 | SEQ ID NO: 9842 | SEQ ID NO: 9867 |
| Spike S2 | SEQ ID NO: 9843 | SEQ ID NO: 9868 |
| Spike S1-S2 | SEQ ID NO: 9844 | SEQ ID NO: 9869 |
| HR1 | SEQ ID NO: 9845 | SEQ ID NO: 9870 |
| HR2 | SEQ ID NO: 9846 | SEQ ID NO: 9871 |
| Orf3Δ100 | SEQ ID NO: 9847 | SEQ ID NO: 9872 |
| Orf4 | SEQ ID NO: 9848 | SEQ ID NO: 9873 |
| Env E | SEQ ID NO: 9849 | SEQ ID NO: 9874 |
| Matrix MΔ100 | SEQ ID NO: 9850 | SEQ ID NO: 9875 |
| Orf7Δ18 | SEQ ID NO: 9851 | SEQ ID NO: 9876 |
| Orf8 | SEQ ID NO: 9852 | SEQ ID NO: 9877 |
| Orf9 | SEQ ID NO: 9853 | SEQ ID NO: 9878 |
| Orf10 | SEQ ID NO: 9854 | SEQ ID NO: 9879 |
| Orf11 | SEQ ID NO: 9855 | SEQ ID NO: 9880 |
| Nucleocapsid N | SEQ ID NO: 9856 | SEQ ID NO: 9881 |
| Orf12 | SEQ ID NO: 9857 | SEQ ID NO: 9882 |

TABLE 29
Cloning, purification and expression in E. coli

| | M.W | | | |
|---|---|---|---|---|
| SARS CoV ORFs | Kd | cloning | Expr. | purification as |
| P28 | 19.7 | + | + | his sol |
| P65 | 70.3 | + | + | his sol |
| Nsp1A (N-term) | 91.6 | + | + | his sol |
| Nsp1B (core) | 80.8 | + | − | |
| Nsp1C (C-term) | 95.3 | + | − | |
| Nsp2A (N-term) | 15.8 | + | + | his ins |
| Nsp2B (C-term) | 15.5 | + | + | his sol |
| Nsp3 | 31.9 | + | − | |
| Nsp4 | 9.1 | + | + | his sol |
| Nsp5 | 21.8 | + | + | his sol |
| Nsp6 | 12.4 | + | + | his sol |
| Nsp7 | 15.3 | + | + | his ins |
| Nsp9A (N-term) | 50.8 | + | − | |
| Nsp9B (C-term) | 51.6 | + | + | his ins |
| Nsp10 | 66 | − | | |
| Nsp11 | 58 | − | | |
| Nsp12 | 38 | − | | |
| Nsp13 | 32.7 | + | + | his ins |
| Spike (S1-his) | 71.3 | + | + | his ins |
| Spike (S2-his) | 58.6 | + | − | |
| Spike (S1S2-his) | 130 | + | + | his ins |
| HR1 | 11 | + | + | his ins |
| HR2 | 5.4 | + | + | his sol |
| ORF3 Δ100¹ | 19.1 | + | − | |
| ORF4 | 16.9 | + | + | his ins (trimer) |
| Envelope (E) | 34.3 | + | + | gst ins (IB) |
| Matrix (M) Δ100 | 13.3 | + | + | his ins |
| ORF7Δ18² | 31 | + | + | gst sol |
| ORF8 | 39.5 | + | + | gst ins (IB) |
| ORF9 | 30.8 | + | + | gst sol |
| ORF10 | 30.3 | + | + | gst ins (IB) |
| ORF11 | 35.2 | + | + | gst ins (IB) |
| Nucleocapsid (N) | 43.6 | + | + | his ins |
| ORF12 | 36.7 | + | + | his ins |

TABLE 30
E. coli expression, purity and yield

| Protein | Tag | Purity (%) | Yield (mg/l) |
|---|---|---|---|
| Nsp2A (N-term) | His | 95 | 1.7 |
| Nsp2B (C-term) | His | 95 | 4.1 |
| Nsp4 | His | 95 | 12.6 |
| Nsp5 | His | 95 | 5.88 |
| Nsp6 | His | 95 | 8.1 |
| P28 | His | 95 | 1 |
| P65 | His | 80 | 0.553 |
| HR2 | His | 95 | 11.9 |
| HR1 | His | 80 | 2.64 |
| Nsp1A | His | 95 | 0.267 |
| Spike S1-S2 | His | 80 | 0.381 |
| Matrix M | His | 85 | 12.4 |
| ORF7 | GST | 85 | 4.9 |

TABLE 31
Primers

| | | | | |
|---|---|---|---|---|
| SEQ ID NO: | Rank | Model | Local | (Position) |

| | | | | |
|---|---|---|---|---|
| 10235 | F1 | 1 | 1 | (106) |
| 10236 | F2 | 2 | 1 | (728) |
| 10237 | F3 | 3 | 1 | (112) |
| 10238 | F4 | 5 | 2 | (1331) |
| 10239 | F5 | 6 | 1 | (12) |
| 10240 | F6 | 6 | 1 | (346) |
| 10241 | F7 | 8 | 1 | (904) |
| 10242 | F8 | 9 | 1 | (1016) |
| 10243 | F9 | 9 | 1 | (1015) |
| 10244 | F10 | 9 | 1 | (719) |
| 10245 | F11 | 9 | 1 | (720) |
| 10246 | F12 | 10 | 1 | (724) |
| 10247 | R1 | 2 | 1 | (1283) |
| 10248 | R2 | 4 | 1 | (756) |
| 10249 | R3 | 4 | 1 | (758) |
| 10250 | R4 | 5 | 2 | (259) |
| 10251 | R5 | 6 | 1 | (54) |
| 10252 | R6 | 7 | 1 | (648) |
| 10253 | R7 | 8 | 1 | (948) |
| 10254 | R8 | 8 | 1 | (260) |
| 10255 | R9 | 9 | 1 | (1282) |
| 10256 | R10 | 9 | 1 | (950) |
| 10257 | R11 | 9 | 1 | (756) |
| 10258 | R12 | 10 | 1 | (132) |

TABLE 32
Primers

| | Scores | | | |
|---|---|---|---|---|
| Rank | Model | Local | Sequence | (Position) |

Primers List: (forward)

| | | | | |
|---|---|---|---|---|
| F1 | 7 | 1 | SEQ ID NO: 10352 | (290) |
| F2 | 7 | 1 | SEQ ID NO: 10353 | (291) |
| F3 | 7 | 1 | SEQ ID NO: 10354 | (294) |
| F4 | 7 | 1 | SEQ ID NO: 10355 | (292) |
| F5 | 7 | 1 | SEQ ID NO: 10356 | (293) |
| F6 | 9 | 1 | SEQ ID NO: 10357 | (198) |
| F7 | 9 | 1 | SEQ ID NO: 10358 | (199) |
| F8 | 10 | 1 | SEQ ID NO: 10359 | (33) |
| F9 | 11 | 1 | SEQ ID NO: 10360 | (200) |
| F10 | 11 | 1 | SEQ ID NO: 10361 | (299) |
| F11 | 12 | 1 | SEQ ID NO: 10362 | (298) |
| F12 | 12 | 1 | SEQ ID NO: 10363 | (297) |
| F13 | 14 | 1 | SEQ ID NO: 10364 | (35) |
| F14 | 14 | 1 | SEQ ID NO: 10365 | (34) |
| F15 | 16 | 1 | SEQ ID NO: 10366 | (300) |
| F16 | 17 | 1 | SEQ ID NO: 10367 | (295) |
| F17 | 17 | 1 | SEQ ID NO: 10368 | (296) |
| F18 | 17 | 1 | SEQ ID NO: 10369 | (175) |
| F19 | 17 | 1 | SEQ ID NO: 10370 | (36) |
| F20 | 20 | 1 | SEQ ID NO: 10371 | (202) |
| F21 | 20 | 1 | SEQ ID NO: 10372 | (201) |
| F22 | 28 | 1 | SEQ ID NO: 10373 | (204) |
| F23 | 28 | 1 | SEQ ID NO: 10374 | (203) |
| F24 | 29 | 1 | SEQ ID NO: 10375 | (269) |
| F25 | 29 | 1 | SEQ ID NO: 10376 | (268) |

Primers List: (reverse)

| | | | | |
|---|---|---|---|---|
| R1 | 7 | 1 | SEQ ID NO: 10377 | (337) |
| R2 | 9 | 1 | SEQ ID NO: 10378 | (229) |
| R3 | 11 | 1 | SEQ ID NO: 10379 | (230) |
| R4 | 11 | 1 | SEQ ID NO: 10380 | (338) |
| R5 | 12 | 1 | SEQ ID NO: 10381 | (207) |
| R6 | 12 | 1 | SEQ ID NO: 10382 | (338) |
| R7 | 13 | 1 | SEQ ID NO: 10383 | (231) |
| R8 | 14 | 1 | SEQ ID NO: 10384 | (80) |
| R9 | 14 | 1 | SEQ ID NO: 10385 | (232) |
| R10 | 15 | 1 | SEQ ID NO: 10386 | (82) |
| R11 | 16 | 1 | SEQ ID NO: 10387 | (340) |
| R12 | 17 | 1 | SEQ ID NO: 10388 | (83) |
| R13 | 17 | 1 | SEQ ID NO: 10389 | (206) |
| R14 | 17 | 1 | SEQ ID NO: 10390 | (82) |
| R15 | 17 | 1 | SEQ ID NO: 10391 | (337) |
| R16 | 18 | 1 | SEQ ID NO: 10392 | (341) |
| R17 | 20 | 1 | SEQ ID NO: 10393 | (340) |
| R18 | 20 | 1 | SEQ ID NO: 10394 | (233) |
| R19 | 21 | 1 | SEQ ID NO: 10395 | (79) |
| R20 | 22 | 1 | SEQ ID NO: 10396 | (213) |
| R21 | 28 | 1 | SEQ ID NO: 10397 | (236) |
| R22 | 29 | 1 | SEQ ID NO: 10398 | (317) |
| R23 | 32 | 1 | SEQ ID NO: 10399 | (391) |
| R24 | 35 | 1 | SEQ ID NO: 10400 | (57) |
| R25 | 36 | 1 | SEQ ID NO: 10401 | (237) |

Primers List (left part): SEQ ID NO^{S}: 10402-10433
Primers List (right part): SEQ ID NO^{S}: 10434-10464
Primers List (forward): SEQ ID NO^{S}: 10465-10484
Primers List (reverse): SEQ ID NO^{S}: 10485-10504

TABLE 33
Primers

| | Scores | | | |
|---|---|---|---|---|
| Rank | Model | Local | Sequence | (Position) |

Primers List: (forward)

| | | | | |
|---|---|---|---|---|
| F1 | 1 | 1 | SEQ ID NO: 10580 | (637) |
| F2 | 2 | 1 | SEQ ID NO: 10581 | (439) |
| F3 | 2 | 1 | SEQ ID NO: 10582 | (440) |
| F4 | 3 | 1 | SEQ ID NO: 10583 | (729) |
| F5 | 4 | 1 | SEQ ID NO: 10584 | (696) |
| F6 | 4 | 1 | SEQ ID NO: 10585 | (697) |
| F7 | 4 | 1 | SEQ ID NO: 10586 | (111) |
| F8 | 5 | 1 | SEQ ID NO: 10587 | (867) |
| F9 | 5 | 1 | SEQ ID NO: 10588 | (868) |
| F10 | 5 | 1 | SEQ ID NO: 10589 | (869) |
| F11 | 5 | 1 | SEQ ID NO: 10590 | (640) |
| F12 | 6 | 1 | SEQ ID NO: 10591 | (438) |
| F13 | 6 | 1 | SEQ ID NO: 10592 | (437) |
| F14 | 6 | 1 | SEQ ID NO: 10593 | (436) |
| F15 | 6 | 1 | SEQ ID NO: 10594 | (732) |
| F16 | 6 | 1 | SEQ ID NO: 10595 | (635) |
| F17 | 6 | 1 | SEQ ID NO: 10596 | (457) |
| F18 | 6 | 1 | SEQ ID NO: 10597 | (458) |
| F19 | 6 | 1 | SEQ ID NO: 10598 | (636) |
| F20 | 7 | 1 | SEQ ID NO: 10599 | (854) |
| F21 | 7 | 1 | SEQ ID NO: 10600 | (855) |
| F22 | 7 | 1 | SEQ ID NO: 10601 | (581) |
| F23 | 7 | 1 | SEQ ID NO: 10602 | (853) |
| F24 | 7 | 1 | SEQ ID NO: 10603 | (342) |
| F25 | 7 | 1 | SEQ ID NO: 10604 | (343) |
| F26 | 7 | 1 | SEQ ID NO: 10605 | (112) |
| F27 | 7 | 1 | SEQ ID NO: 10606 | (94) |
| F28 | 7 | 1 | SEQ ID NO: 10607 | (642) |
| F29 | 8 | 1 | SEQ ID NO: 10608 | (638) |
| F30 | 8 | 1 | SEQ ID NO: 10609 | (639) |
| F31 | 8 | 1 | SEQ ID NO: 10610 | (730) |
| F32 | 8 | 1 | SEQ ID NO: 10611 | (641) |
| F33 | 8 | 1 | SEQ ID NO: 10612 | (731) |
| F34 | 8 | 1 | SEQ ID NO: 10613 | (326) |
| F35 | 8 | 1 | SEQ ID NO: 10614 | (325) |
| F36 | 9 | 1 | SEQ ID NO: 10615 | (517) |
| F37 | 9 | 1 | SEQ ID NO: 10616 | (701) |
| F38 | 9 | 1 | SEQ ID NO: 10617 | (208) |
| F39 | 9 | 1 | SEQ ID NO: 10618 | (209) |
| F40 | 9 | 1 | SEQ ID NO: 10619 | (702) |
| F41 | 9 | 1 | SEQ ID NO: 10620 | (210) |
| F42 | 10 | 1 | SEQ ID NO: 10621 | (634) |
| F43 | 10 | 1 | SEQ ID NO: 10622 | (694) |
| F44 | 10 | 1 | SEQ ID NO: 10623 | (693) |
| F45 | 10 | 1 | SEQ ID NO: 10624 | (728) |
| F46 | 10 | 1 | SEQ ID NO: 10625 | (695) |
| F47 | 10 | 1 | SEQ ID NO: 10626 | (95) |
| F48 | 11 | 1 | SEQ ID NO: 10627 | (455) |
| F49 | 11 | 1 | SEQ ID NO: 10628 | (456) |
| F50 | 11 | 1 | SEQ ID NO: 10629 | (454) |

Primers List: (reverse)

| | | | | |
|---|---|---|---|---|
| R1 | 1 | 1 | SEQ ID NO: 10630 | (367) |
| R2 | 1 | 1 | SEQ ID NO: 10631 | (666) |
| R3 | 2 | 1 | SEQ ID NO: 10632 | (464) |
| R4 | 3 | 1 | SEQ ID NO: 10633 | (669) |
| R5 | 3 | 1 | SEQ ID NO: 10634 | (750) |
| R6 | 4 | 1 | SEQ ID NO: 10635 | (720) |
| R7 | 4 | 1 | SEQ ID NO: 10636 | (465) |
| R8 | 4 | 1 | SEQ ID NO: 10637 | (370) |
| R9 | 4 | 1 | SEQ ID NO: 10638 | (668) |
| R10 | 4 | 1 | SEQ ID NO: 10639 | (135) |
| R11 | 5 | 1 | SEQ ID NO: 10640 | (901) |
| R12 | 5 | 1 | SEQ ID NO: 10641 | (667) |
| R13 | 6 | 1 | SEQ ID NO: 10642 | (609) |
| R14 | 6 | 1 | SEQ ID NO: 10643 | (464) |
| R15 | 6 | 1 | SEQ ID NO: 10644 | (665) |
| R16 | 6 | 1 | SEQ ID NO: 10645 | (486) |
| R17 | 6 | 1 | SEQ ID NO: 10646 | (356) |
| R18 | 6 | 1 | SEQ ID NO: 10647 | (758) |
| R19 | 7 | 1 | SEQ ID NO: 10648 | (366) |
| R20 | 7 | 1 | SEQ ID NO: 10649 | (368) |
| R21 | 7 | 1 | SEQ ID NO: 10650 | (136) |
| R22 | 7 | 1 | SEQ ID NO: 10651 | (675) |
| R23 | 7 | 1 | SEQ ID NO: 10652 | (366) |
| R24 | 7 | 1 | SEQ ID NO: 10653 | (608) |
| R25 | 7 | 1 | SEQ ID NO: 10654 | (884) |
| R26 | 7 | 1 | SEQ ID NO: 10655 | (120) |
| R27 | 8 | 1 | SEQ ID NO: 10656 | (355) |
| R28 | 8 | 1 | SEQ ID NO: 10657 | (671) |
| R29 | 8 | 1 | SEQ ID NO: 10658 | (756) |
| R30 | 8 | 1 | SEQ ID NO: 10659 | (751) |
| R31 | 8 | 1 | SEQ ID NO: 10660 | (666) |
| R32 | 9 | 1 | SEQ ID NO: 10661 | (242) |
| R33 | 9 | 1 | SEQ ID NO: 10662 | (543) |
| R34 | 9 | 1 | SEQ ID NO: 10663 | (724) |
| R35 | 9 | 1 | SEQ ID NO: 10664 | (482) |
| R36 | 10 | 1 | SEQ ID NO: 10665 | (121) |
| R37 | 10 | 1 | SEQ ID NO: 10666 | (662) |
| R38 | 10 | 1 | SEQ ID NO: 10667 | (750) |
| R39 | 10 | 1 | SEQ ID NO: 10668 | (719) |
| R40 | 10 | 1 | SEQ ID NO: 10669 | (242) |
| R41 | 11 | 1 | SEQ ID NO: 10670 | (484) |
| R42 | 11 | 1 | SEQ ID NO: 10671 | (375) |
| R43 | 11 | 1 | SEQ ID NO: 10672 | (728) |
| R44 | 11 | 1 | SEQ ID NO: 10673 | (373) |
| R45 | 11 | 1 | SEQ ID NO: 10674 | (998) |
| R46 | 11 | 1 | SEQ ID NO: 10675 | (486) |
| R47 | 12 | 1 | SEQ ID NO: 10676 | (881) |
| R48 | 12 | 1 | SEQ ID NO: 10677 | (882) |
| R49 | 12 | 1 | SEQ ID NO: 10678 | (244) |
| R50 | 12 | 1 | SEQ ID NO: 10679 | (1003) |

Primers List (left part): SEQ ID NO^{S}: 10680-10974
Primers List (right part): SEQ ID NO^{S}: 10975-11282
Primers List (forward): SEQ ID NO^{S}: 11283-11302
Primers List (reverse): SEQ ID NO^{S}: 11303-11322

TABLE 34

| Compound | | | |
|---|---|---|---|
| # | Structure | Name | MH+ |
| 1 | 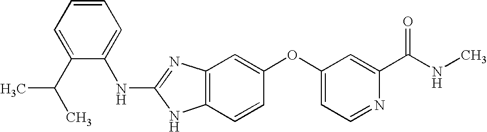 | N-methyl-4-[(2-{[2-(1- methylethyl)phenyl]amino}-1H- benzimidazol-5-yl)oxy]pyridine- 2-carboxamide | 402.5 |
| 2 | 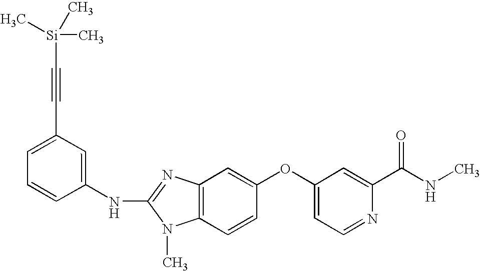 | N-methyl-4-{[1-methyl-2-({3- [(trimethylsilyl)ethynyl]phenyl}amino)-1H-benzimidazol-5- yl]oxy}pyridine-2-carboxamide | 470.6 |
| 3 | 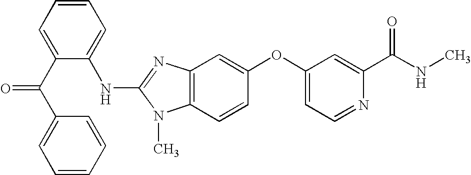 | N-methyl-4-[(1-methyl-2-{[2- (phenylcarbonyl)phenyl]amino}- 1H-benzimidazol-5- yl)oxy]pyridine-2-carboxamide | 478.5 |
| 4 | 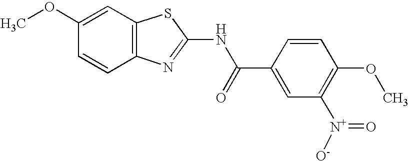 | 4-(methyloxy)-N-[6- (methyloxy)-1,3-benzothiazol-2- yl]-3-nitrobenzamide | 360.4 |
| 5 | 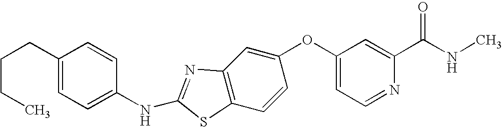 | 4-({2-[(4-butylphenyl)amino]- 1,3-benzothiazol-5-yl}oxy)-N- methylpyridine-2-carboxamide | 433.5 |
| 6 | 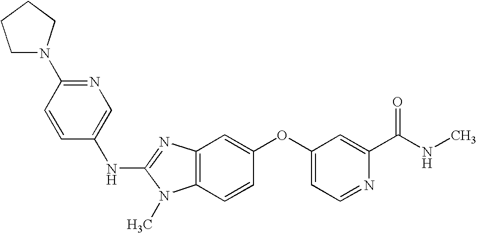 | N-methyl-4-({1-methyl-2-[(6- pyrrolidin-1-ylpyridin-3-yl) amino]-1H-benzimidazol-5- yl}oxy)pyridine-2-carboxamide | 444.5 |
| 7 | 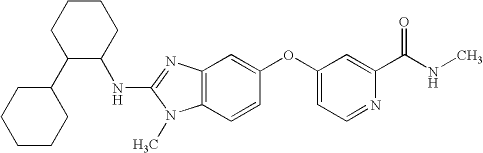 | 4-({2-[1,1′-bi(cyclohexyl)-2- ylamino]-1-methyl-1H- benzimidazol-5-yl}oxy)-N- methylpyridine-2-carboxamide | 462.6 |
| 8 | 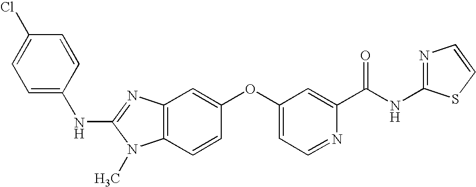 | 4-({2-[(4-chlorophenyl)amino]- 1-methyl-1H-benzimidazol-5- yl}oxy)-N-1,3-thiazol-2- ylpyridine-2-carboxamide | 477.9 |
| 9 | 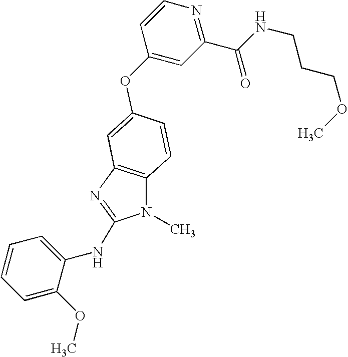 | 4-[(1-methyl-2-{[2-(methyloxy) phenyl]amino}-1H- benzimidazol-5-yl)oxy]-N-[3- (methyloxy)propyl]pyridine-2- carboxamide | 462.5 |
| 10 | 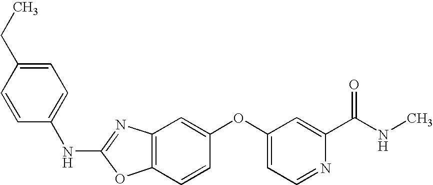 | 4-({2-[(4-ethylphenyl)amino]- 1,3-benzoxazol-5-yl}oxy)-N- methylpyridine-2-carboxamide | 389.4 |
| 11 | 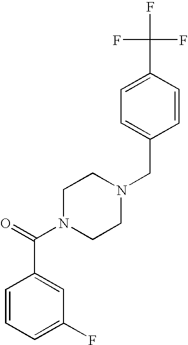 | 1-[(3-fluorophenyl)carbonyl]-4- {[4-(trifluoromethyl)phenyl]methyl}pierazine | 367.4 |
| 12 | 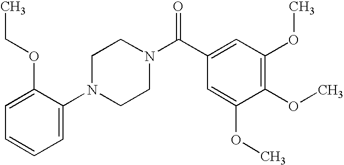 | 1-[2-(ethyloxy)phenyl]-4- {[3,4,5-tris(methyloxy)phenyl]carbonyl}piperazine | 401.5 |
| 13 | 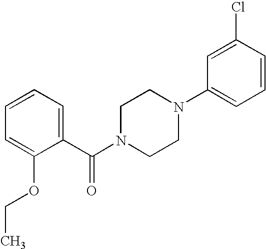 | 1-(3-chlorophenyl)-4-{[2- (ethyloxy)phenyl]carbonyl}piperazine | 345.8 |
| 14 | 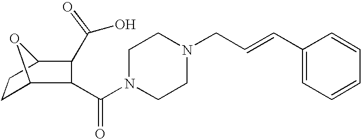 | 3-({4-[(2E)-3-phenylprop-2- enyl]piperazin-1-yl}carbonyl)-7- oxabicyclo[2.2.1]heptane-2- carboxylic acid | 371.4 |
| 15 | 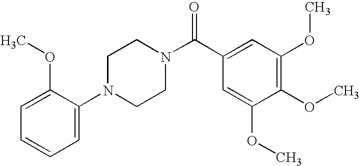 | 1-[2-(methyloxy)phenyl]-4- {[3,4,5-tris(methyloxy) phenyl]carbonyl}piperazine | 387.4 |
| 16 | 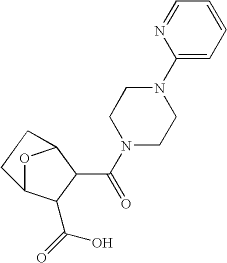 | 3-[(4-pyridin-2-ylpiperazin-1- yl)carbonyl]-7-oxabicyclo[2.2.1]heptane-2-carboxylic acid | 332.4 |
| 17 | 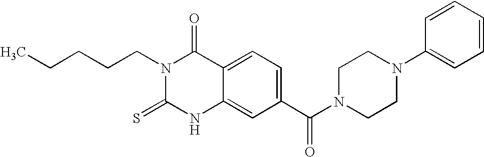 | 3-pentyl-7-[(4-phenylpiperazin- 1-yl)carbonyl]-2-thioxo-2,3- dihydroquinazolin-4(1H)-one | 437.6 |
| 18 | 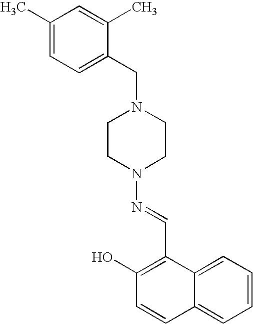 | 1-[(E)-({4-[(2,4-dimethyl- phenyl)methyl]piperazin-1-yl}imino)methyl]naphthalen-2-ol | 374.5 |
| 19 | 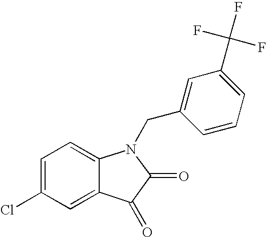 | 5-chloro-1-{[3- (trifluoromethyl)phenyl]methyl)-1H-indole-2,3-dione | 340.7 |
| 20 | 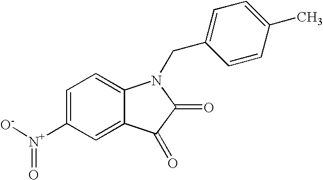 | 1-[(4-methylphenyl)methyl]-5- nitro-1H-indole-2,3-dione | 297.3 |
| 21 | 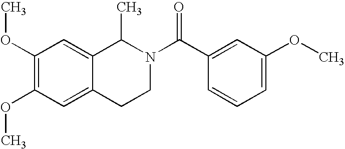 | 1-methyl-6,7-bis(methyloxy)-2- {[3-(methyloxy)phenyl]carbonyl}-1,2,3,4- tetrahydroisoquinoline | 342.4 |
| 22 | 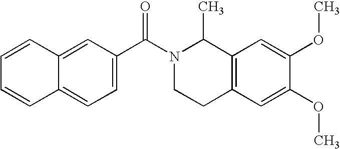 | 1-methyl-6,7-bis(methyloxy)-2- (naphthalen-2-ylcarbonyl)- 1,2,3,4-tetrahydroisoquinoline | 362.4 |
| 23 | 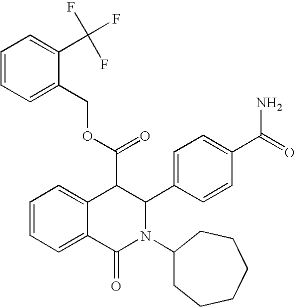 | [2-(trifluoromethyl)phenyl]methyl 3-[4-(aminocarbonyl) phenyl]-2-cycloheptyl-1-oxo- 1,2,3,4-tetrahydroisoquinoline- 4-carboxylate | 565.6 |
| 24 | 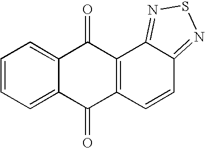 | anthra[1,2,5]thiadiazole- 6,11-dione | 267.3 |
| 25 | 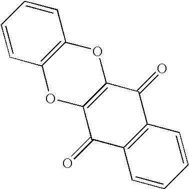 | benzo[b]oxanthrene-6,11-dione | 265.2 |
| 26 | 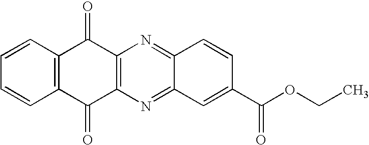 | ethyl 6,11-dioxo-6,11- dihydrobenzo[b]phenazine-2- carboxylate | 333.3 |
| 27 | 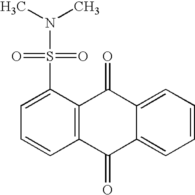 | N,N-dimethyl-9,10-dioxo-9,10- dihydroanthracene-1- sulfonamide | 316.3 |
| 28 | 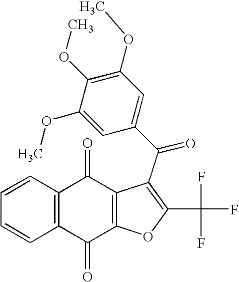 | 2-(trifluoromethyl)-3-{[3,4,5- tris(methyloxy)phenyl]carbonyl}naphtho[2,3-b]furan-4,9-dione | 461.4 |
| 29 | 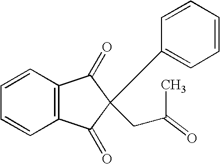 | 2-(2-oxopropyl)-2-phenyl-1H- indene-1,3(2H)-dione | 279.3 |
| 30 | 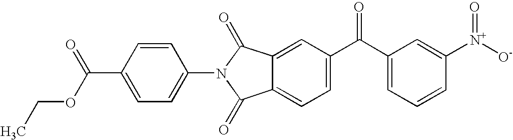 | ethyl 4-{5-[(3- nitrophenyl)carbonyl]-1,3- dioxo-1,3-dihydro-2H-isoindol- 2-yl}benzoate | 445.4 |
| 31 | 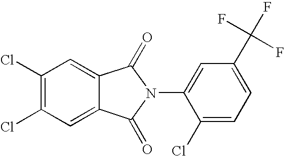 | 5,6-dichloro-2-[2-chloro-5- (trifluoromethyl)phenyl]-1H- isoindole-1,3(2H)-dione | 395.6 |
| 32 | 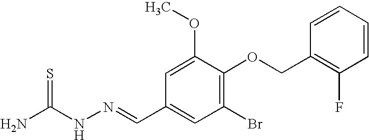 | 3-bromo-4-{[(2- fluorophenyl)methyl]oxy}-5- (methyloxy)benzaldehyde thiosemicarbazone | 413.3 |
| 33 | 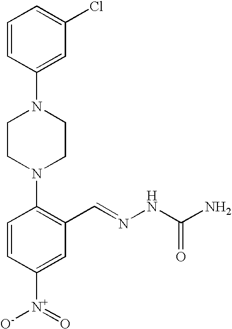 | 2-[4-(3-chlorophenyl)piperazin- 1-yl]-5-nitrobenzaldehyde thiosemicarbazone | 419.9 |
| 34 | 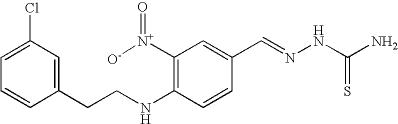 | 4-{[2-(3-chlorophenyl) ethyl]amino}-3- nitrobenzaldehyde thiosemicarbazone | 378.9 |
| 35 | 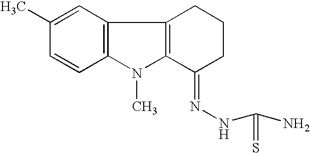 | (1E)-6,9-dimethyl-2,3,4,9- tetrahydro-1H-carbazol-1-one thiosemicarbazone | 287.4 |
| 36 | 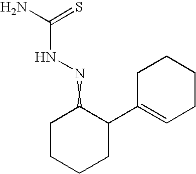 | (2E)-1,1′-bi(cyclohexan)-1-en-2- one thiosemicarbazone | 252.4 |
| 37 | 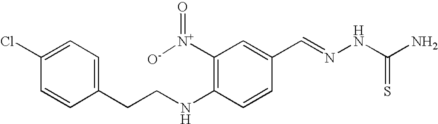 | 4-{[2-(4-chlorophenyl) ethyl]amino}-3-nitro- benzaldehyde thiosemicarbazone | 378.9 |
| 38 | 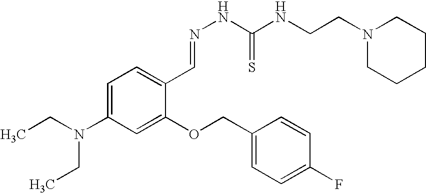 | 4-(diethylamino)-2-{[(4- fluorophenyl)methyl]oxy}benzaldhyde N-(2-piperidin-1- ylethyl)thiosemicarbazone | 486.7 |
| 39 | 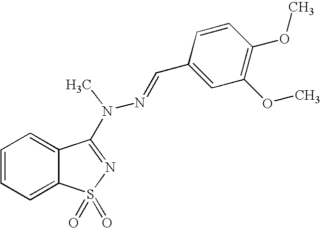 | 3,4-bis(methyloxy)benzaldehyde (1,1-dioxido-1,2-benzisothiazol- 3-yl)(methyl)hydrazone | 360.4 |
| 40 | 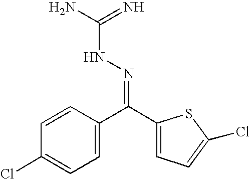 | (2E)-2-[(4-chlorophenyl)(5- chlorothien-2-yl)methylidene]hydrazinecarboximidamide | 314.2 |
| 41 | 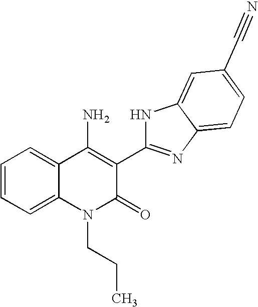 | 2-(4-amino-2-oxo-1-propyl-1,2- dihydroquinolin-3-yl)-1H- benzimidazole-6-carbonitrile | 344.4 |
| 42 | 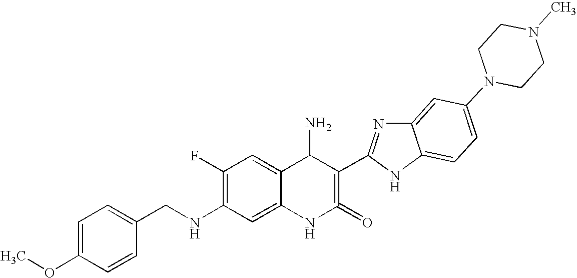 | 4-amino-6-fluoro-7-({[4- (methyloxy)phenyl]methyl}amino)-3-[5-(4-methylpiperazin- 1-yl)-1H-benzimidazol-2-yl]quinolin-2(1H)-one | 528.6 |
| 43 | 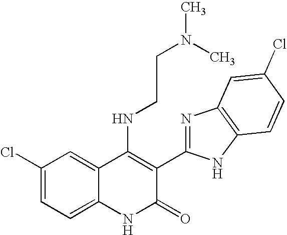 | 6-chloro-3-(5-chloro-1H- benzimidazol-2-yl)-4-{[2- (dimethylamino)ethyl]amino}quinolin-2(1H)-one | 417.3 |
| 44 | 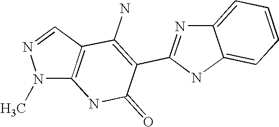 | 4-amino-5-(1H-benzimidazol-2- yl)-1-methyl-1,7-dihydro-6H- pyrazolo[3,4-b]pyridin-6-one | 281.3 |
| 45 | 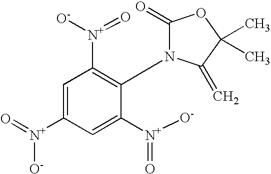 | 5,5-dimethyl-4-methylidene-3- (2,4,6-trinitrophenyl)-1,3- oxazolidin-2-one | 339.2 |
| 46 | 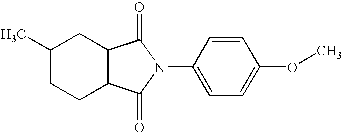 | 5-methyl-2-[4- (methyloxy)phenyl]hexahydro- 1H-isoindole-1,3(2H)-dione | 274.3 |
| 47 | 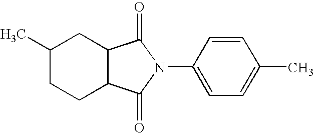 | 5-methyl-2-(4- methylphenyl)hexahydro-1H- isoindole-1,3(2H)-dione | 258.3 |
| 48 | 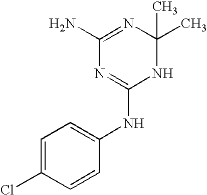 | N-2˜-(4-chlorophenyl)-6,6- dimethyl-1,6-dihydro-1,3,5- triazine-2,4-diamine | 252.7 |
| 49 | 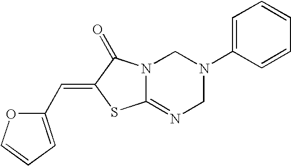 | (7Z)-7-(furan-2-ylmethylidene)- 3-phenyl-3,4-dihydro-2H- [1,3]thiazolo[3,2-a][1,3,5]triazin-6(7H)-one | 312.4 |
| 50 | 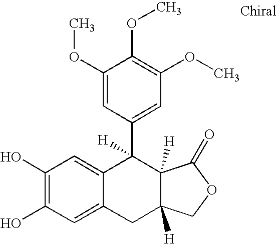 | (3aR,9R,9aR)-6,7-dihydroxy-9- [3,4,5-tris(methyloxy)phenyl]- 3a,4,9,9a-tetrahydronaphtho[2,3- c]furan-1(3H)-one | 387.4 |
| 51 | 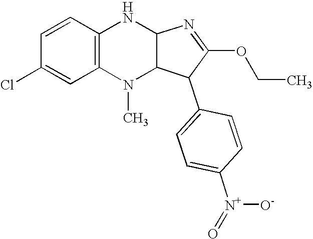 | 6-chloro-2-(ethyloxy)-4-methyl- 3-(4-nitrophenyl)-3a,4,9,9a- tetrahydro-3H-pyrrolo[2,3- b]quinoxaline | 387.8 |
| 52 | 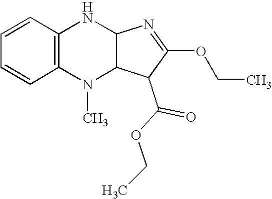 | ethyl 2-(ethyloxy)-4-methyl- 3a,4,9,9a-tetrahydro-3H- pyrrolo[2,3-b]quinoxaline-3- carboxylate | 304.4 |
| 53 | 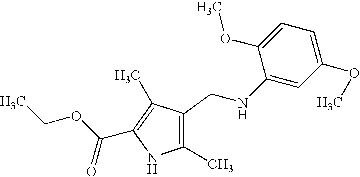 | ethyl 4-({[2,5-bis(methyloxy) phenyl]amino}methyl)- 3,5-dimethyl-1H-pyrrole-2- carboxylate | 333.4 |
| 54 | 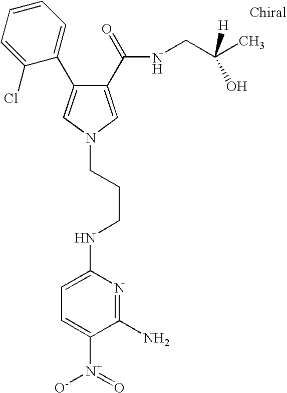 | 1-{3-[(6-amino-5-nitropyridin-2- yl)amino]propyl}-4-(2- chlorophenyl)-N-[(2S)-2- hydroxypropyl]-1H-pyrrole-3- carboxamide | 473.9 |
| 55 | 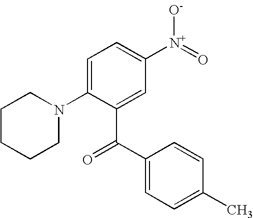 | (4-methylphenyl)(5-nitro-2- piperidin-1-ylphenyl)methanone | 325.4 |
| 56 | 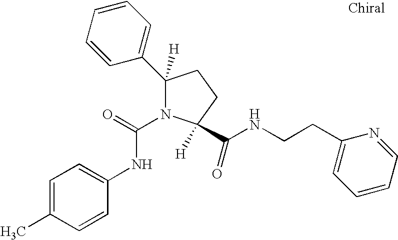 | (2S,5R)-N˜1˜-(4-methyl- phenyl)-5-phenyl-N˜2˜-(2- pyridin-2-ylethyl)pyrrolidine- 1,2-dicarboxamide | 429.5 |
| 57 | 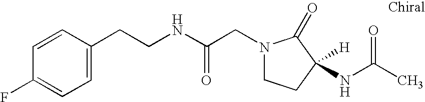 | 2-[(3S)-3-(acetylamino)-2- oxopyrrolidin-1-yl]-N-[2-(4- fluorophenyl)ethyl]acetamide | 322.4 |
| 58 | 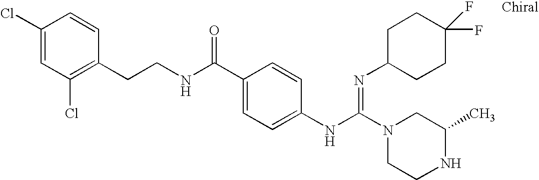 | N-[2-(2,4-dichlorophenyl)ethyl]- 4-({(Z)-[(4,4-difluoro- cyclohexyl)imino][(3S)-3- methylpiperazin-1- yl]methyl}amino)benzamide | 553.5 |
| 59 | 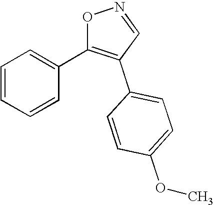 | 4-[4-(methyloxy)phenyl]-5- phenylisoxazole | 252.3 |
| 60 | 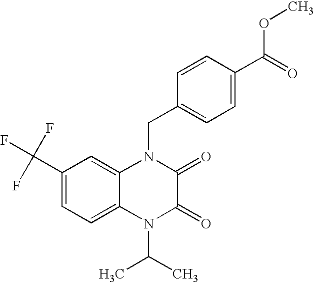 | methyl 4-{[4-(1-methylethyl)- 2,3-dioxo-7-(trifluoromethyl)- 3,4-dihydroquinoxalin-1(2H)- yl]methyl}benzoate | 421.4 |
| 61 | 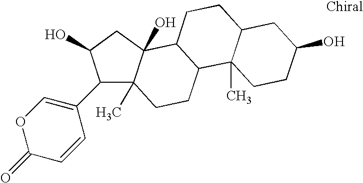 | (3beta, 16beta)-3,14,16- trihydroxybufa-20,22-dienolide | 403.5 |
| 62 | 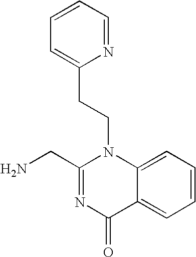 | 2-(aminomethyl)-1-(2-pyridin-2- ylethyl)quinazolin-4(1H)-one | 281.3 |
| 63 | 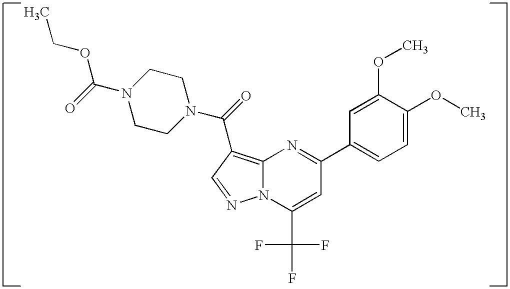 | ethyl 4-{[5-[3,4-bis(methyloxy) phenyl]-7-(trifluoromethyl) pyrazolo[1,5-a]pyrimidin-3- yl]carbonyl}piperazine-1- carboxylate | 508.5 |
| 64 | 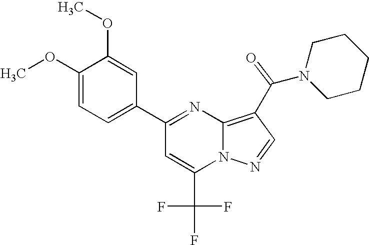 | 5-[3,4-bis(methyloxy)phenyl]-3- (piperidin-1-ylcarbonyl)-7- (trifluoromethyl)pyrazolo[1,5- a]pyrimidine | 435.4 |
| 65 | 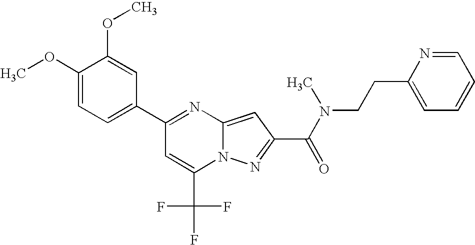 | 5-[3,4-bis(methyloxy)phenyl]-N- methyl-N-(2-pyridin-2-ylethyl)- 7-(trifluoromethyl)pyrazolo[1,5- a]pyrimidine-2-carboxamide | 486.5 |
| 66 | 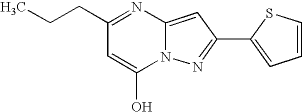 | 5-propyl-2-thien-2- ylpyrazolo[1,5-a]pyrimidin-7-ol | 260.3 |

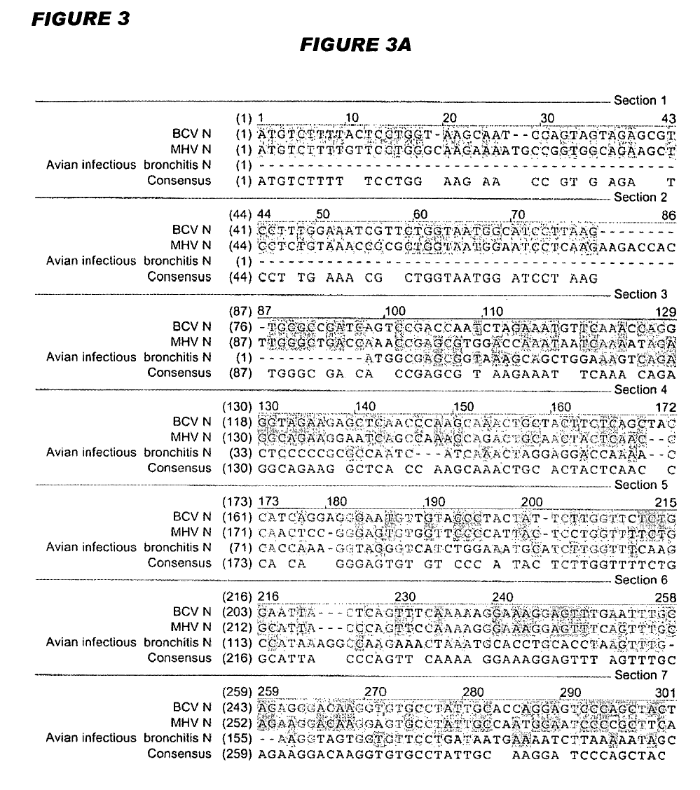

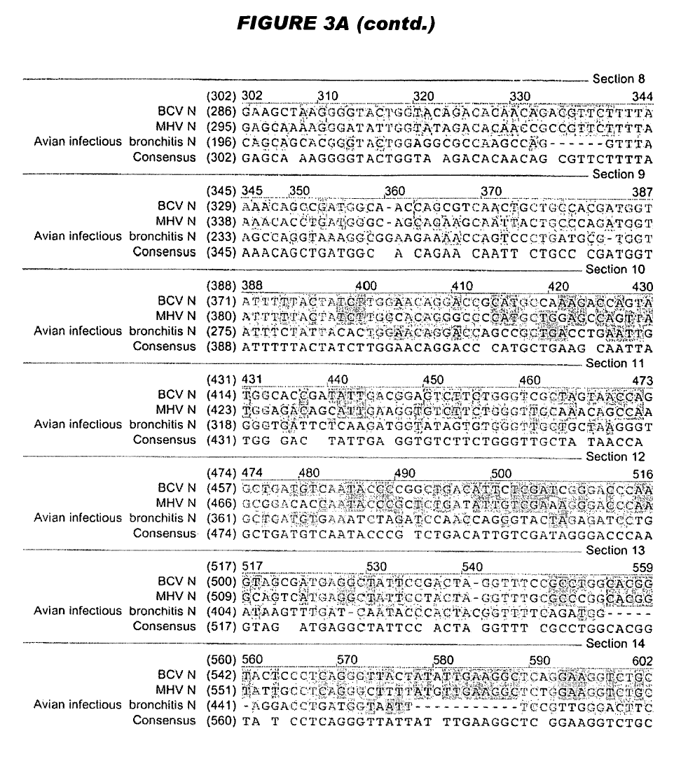

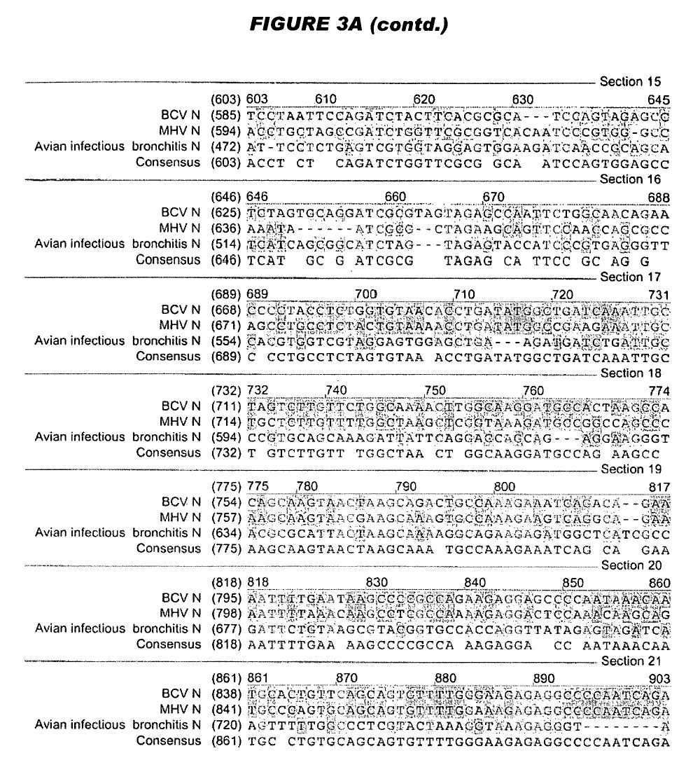

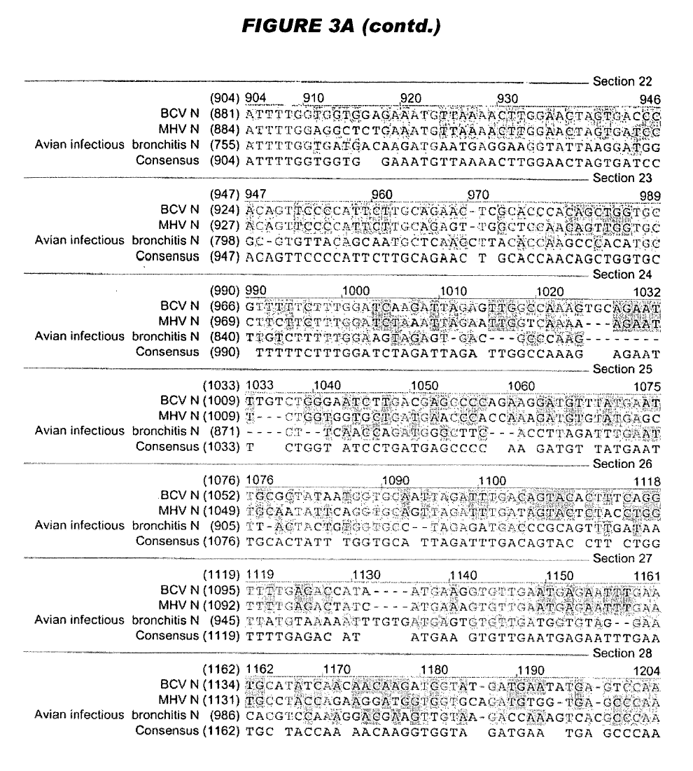

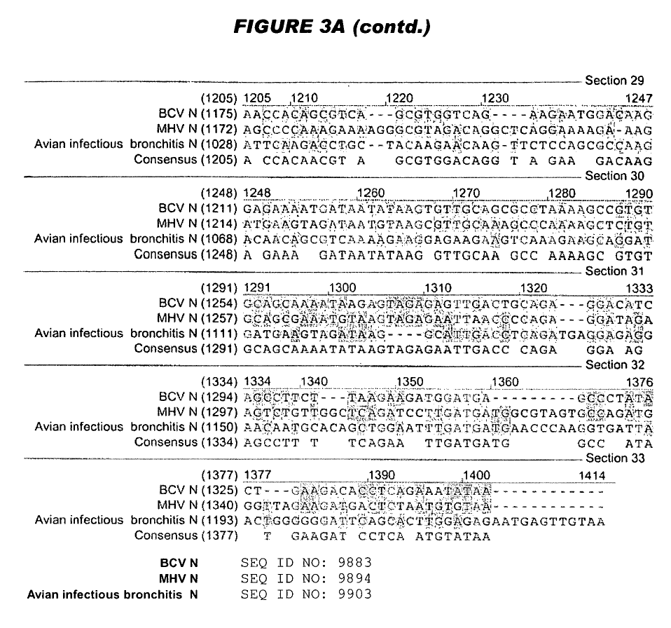

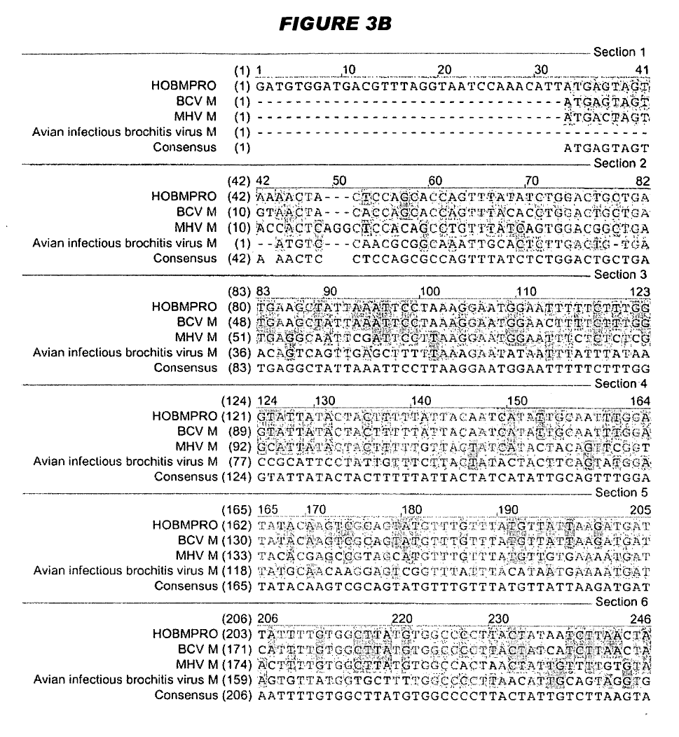

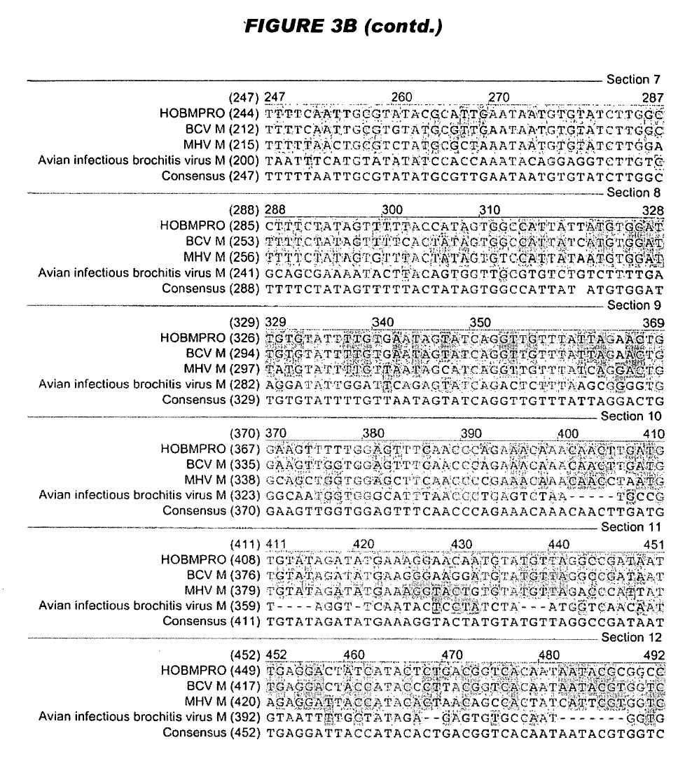

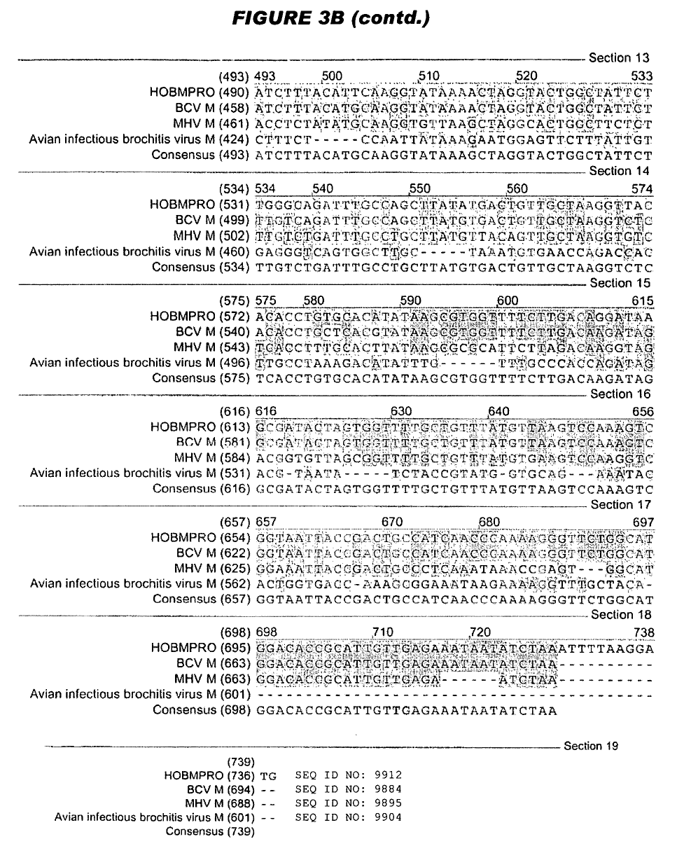

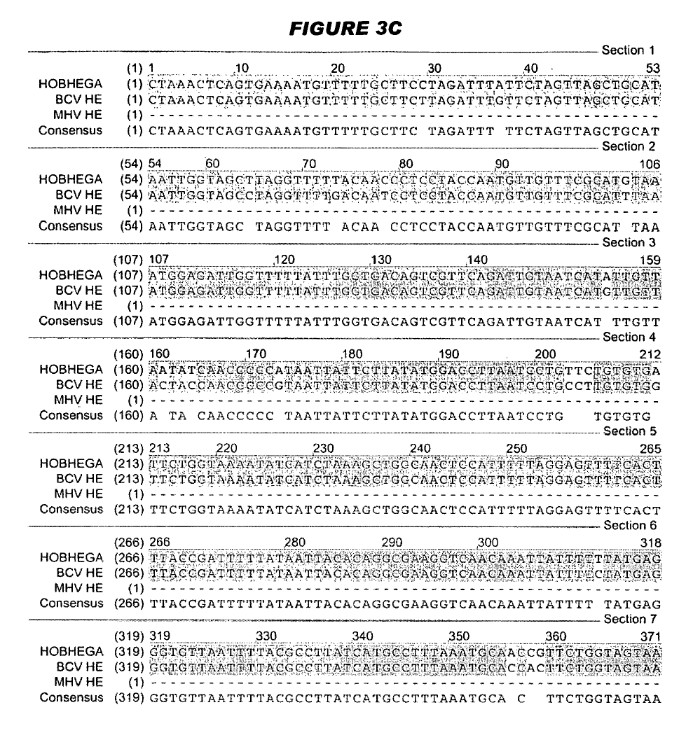

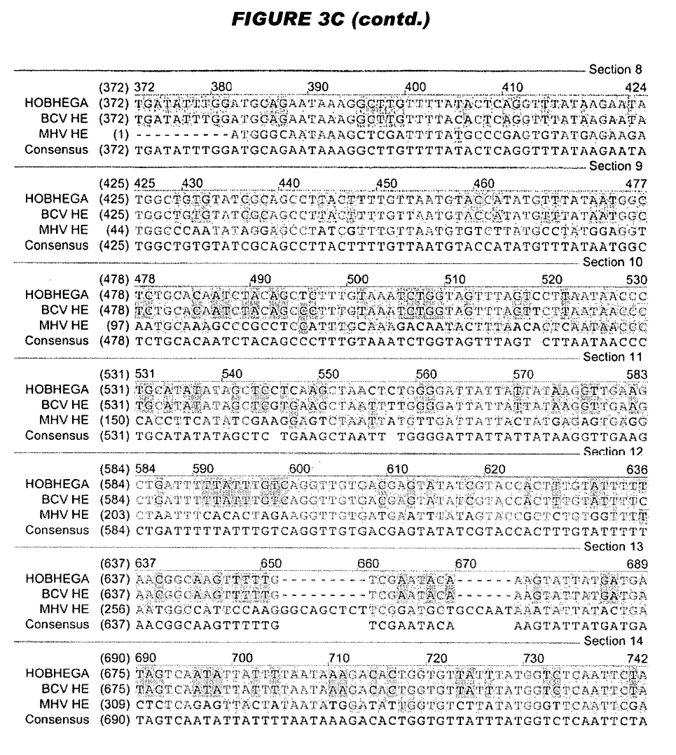

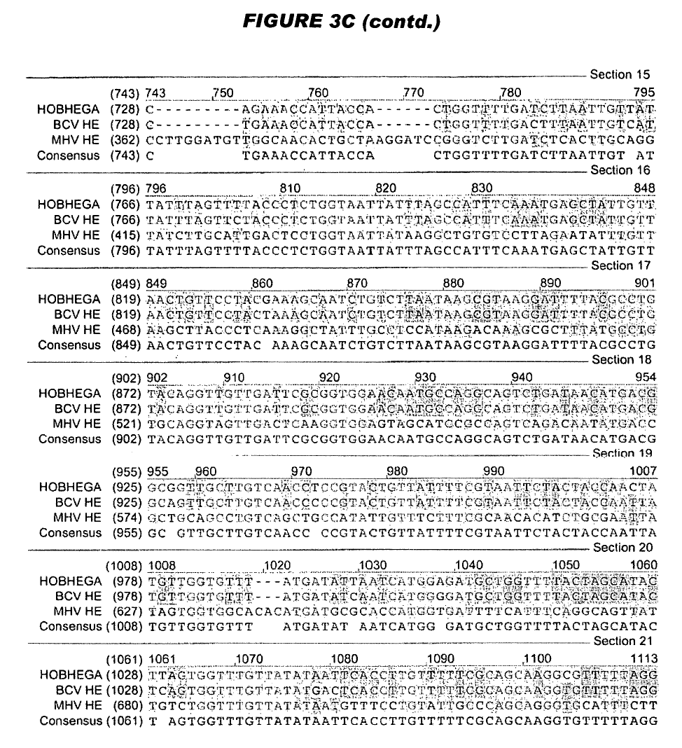

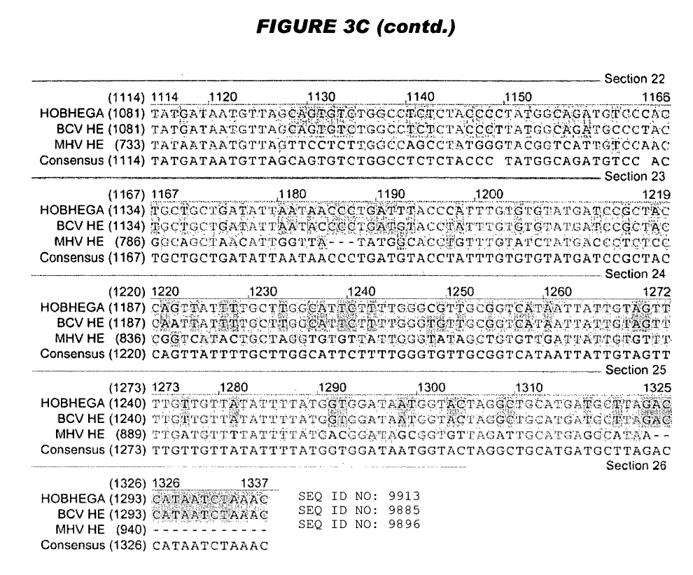

BRIEF DESCRIPTION OF SEQUENCE LISTING

| SEQ ID NO: | Description |
|---|---|
| 1 | Draft genome assembly from The Genome Science Center in British Colombia, |
| | Canada of sequence from TOR2 isolate. TOR2_draft_genome_assembly_120403 Release 1 |
| 2 | CDC SARS-CoV strain sequence. Entire nucleotide sequence (Urbani strain) |
| 3-20 | Group-specific coronavirus gene products |
| | Feline infectious peritonitis virus (FIPV) |
| | 3/4 = ORF 3b; 5/6 = ORF 3X; 7/8 = ORF 3A |
| | Canine coronavirus |
| | 9/10 = ORF 7b; 11/12 = ORF 7a |
| | Avian infectious bronchitis virus |
| | 13/14 = ORF 5b; 15/16 = ORF 5a; 17/18 = ORF 3a; 19/20 = ORF 3b |
| 21-520 | 500 primers for left part |
| 521-1020 | 500 primers for right part |
| 1021-3520 | Forward primers from Table 4 |
| 3521-6020 | Reverse primers from Table 4 |
| 6021-6026 | FIG. 9 primers |
| 6027-6033 | FIG. 11 primers |
| 6034-6038 | Five primers from http://content.nejm.org/cgi/reprint/NEJMoa030781v2.pdf |
| 6039-6051 | PEP1 to PEP13 |
| 6052 | Extended PEP13 |
| 6053-6056 | 229E human coronavirus sequences |
| 6057-6060 | TGV sequences |
| 6061-6064 | PEDV sequences |
| 6065-6068 | Bovine coronavirus sequences |
| 6069-6071 | Murine hepatitis virus sequences |
| 6072-6075 | AIBV sequences |
| 6076-6170 | Primer sequences (forward) |
| 6171-6265 | Primer sequences (reverse) |
| 6266-6304 | Primer sequences (forward) |
| 6305-6343 | Primer sequences (reverse) |
| 6344-6366 | Primer sequences (forward) |
| 6367-6392 | Primer sequences (reverse) |
| 6393-6440 | Primer sequences (forward) F1-F48 |
| 6441-6487 | Primer sequences (reverse) R1-R47 |
| 6488-6559 | Primer sequences |
| 6560-6568 | Primer sequences |
| 6569 | The nsp2 proteinase (3CL-PRO) sequence in SARS coronavirus |
| 6570-72 | The nsp2 proteinases (3CLp) of avian IBV, MHV, and BCoV |
| 6573 | Consensus nsp2 proteinases sequence |
| 6574-6577 | IG sequences from FIG. 18 |
| 6578 | Expression construct of nSh in pCMVIII |
| 6579 | Expression construct of nS in pCMVIII |
| 6580 | Expression construct of nSh ΔTC in pCMVIII |
| 6581 | Expression construct of nS ΔTC in pCMVIII |
| 6582 | Expression construct of nS1h in pCMVIII |
| 6583 | Expression construct of nS1 in pCMVIII |
| 6584-6585 | Primers for cDNA amplification |
| 6585-6587 | Primers for RT-PCR |
| 6588-6809 | Component sequences of FIG. 23 (≧4 amino acids) |
| 6810-7179 | Component sequences of FIG. 24 (≧4 amino acids) |
| 7180-7187 | N-glycosylation sites within SEQ ID NO: 6039 |
| 7188-7189 | Component sequences of FIG. 25 |
| 7190 | Fragment of SEQ ID NO: 7188 |
| 7191 | Polynucleotide encoding SEQ ID NO: 7190 |
| 7192 | Amino acids 879-1005 of SEQ ID NO: 6042 |
| 7193 | Amino acids 879-980 of SEQ ID NO: 6042 |
| 7194 | Amino acids 901-1005 of SEQ ID NO: 6042 |
| 7195 | Amino acids 1144-1201 of SEQ ID NO: 6042 |
| 7196 | Amino acids 1144-1196 of SEQ ID NO: 6042 |
| 7197-7199 | Membrane fusion peptide regions |
| 7200-7206 | NadA-based polypeptides |
| 7207-7223 | N-glycosylation sites within SEQ ID NO: 6042 |
| 7224-7231 | Slippage region |
| 7232 | Orflab polyprotein |
| 7233-7244 | Orflab polyproteins |
| 7245-7247 | X₂ sequences for SEQ ID NOS 7233-7244 |
| 7248-7253 | Orflab polyproteins |
| 7254 | Zinc binding region 2 site |
| 7255-7271 | N-glycosylation sites in SEQ ID NOS: 6040-41, 6043, 6045-46, 6050-51 |
| 7272-7291 | Polypeptides and polynucleotides |
| 7292-7293 | Intergenic sequences |
| 7294-7301 | Nucleotides from 5′ end of SARSV genome followed by intergenic sequence |
| 7302-7306 | NadA constructs |
| 7307-7308 | Fragments of SEQ ID NO: 6042 |
| 7309 | NadA sequence |
| 7310-7311 | NadA leader sequences |
| 7312-7315 | Amino acid sequencess from NadA |
| 7316-7324 | PCR primers |
| 7325-7330 | Primers |
| 7331 | CCACC sequence |
| 7332-7336 | 3′ UTR forward primers |
| 7337-7341 | 3′ UTR reverse primers |
| 7342-7352 | 3′ UTR probes |
| 7353-7362 | 5′ UTR forward primers |
| 7363-7373 | 5′ UTR reverse primers |
| 7374-7385 | 5′ UTR probes |
| 7386 | Conserved octanucleotide |
| 7387 | Reverse complement of SEQ ID NO: 7293 |
| 7388 | Intergenic sequence |
| 7389 | Poly T |
| 7390 | Stem-loop sequence |
| 7391-7392 | Poly-glycine linkers |
| 7393 | Poly-histidine tag |
| 7394 | Nucleocapsid epitope site |
| 7395 | Antisense primer |
| 7396-7397 | Probes |
| 7398-7399 | Antigenic fragments of SEQ ID NO: 6042 |
| 7400-7639 | T-epitope analysis of SEQ ID NO: 6039 |
| 7640-7800 | T-epitope analysis of SEQ ID NO: 6040 |
| 7801-8040 | T-epitope analysis of SEQ ID NO: 6041 |
| 8041-8280 | T-epitope analysis of SEQ ID NO: 6042 |
| 8281-8486 | T-epitope analysis of SEQ ID NO: 6043 |
| 8487-8665 | T-epitope analysis of SEQ ID NO: 6044 |
| 8666-8820 | T-epitope analysis of SEQ ID NO: 6045 |
| 8821-9018 | T-epitope analysis of SEQ ID NO: 6046 |
| 9019-9131 | T-epitope analysis of SEQ ID NO: 6047 |
| 9132-9308 | T-epitope analysis of SEQ ID NO: 6048 |
| 9309-9437 | T-epitope analysis of SEQ ID NO: 6049 |
| 9438-9538 | T-epitope analysis of SEQ ID NO: 6050 |
| 9539-9752 | T-epitope analysis of SEQ ID NO: 6052 |
| 9753-9763 | Primers for spike protein amplification, particularly fragments of spike |
| 9764-9765 | N-glycosylation sites within SEQ ID NO: 6039 |
| 9766-9779 | Cleavage products for ORFlab (Table 10) |
| 9780-9782 | Forward primer, reverse primer, probe |
| 9783-9784 | Lysine-rich region |
| 9785-9798 | Oligonucleotides used for S. cerevisiae expression |
| 9799-9802 | Sequences from FIGS. 65 & 66 |
| 9803-9882 | Primers for E. coli cloning |
| 9883-9885 | BCV nucleotide sequences for FIGS. 3A, 3B, 3C |
| 9886-9891 | BCV amino acid sequences for FIGS. 4A, 4B, 4C, 4D, 4E, 4F |
| 9892 | BCV 5′ UTR |
| 9893 | BCV 3′ UTR |
| 9894-9896 | MHV nucleotide sequences for FIGS. 3A, 3B, 3C |
| 9897-9902 | MHV amino acid sequences for FIGS. 4A, 4B, 4C, 4D, 4E, 4F |
| 9903-9904 | AIBV nucleotide sequences for FIGS. 3A, 3B |
| 9905-9909 | AIBV amino acid sequences for FIGS. 4A, 4B, 4D, 4E, 4F |
| 9910 | AIBV 5′ UTR |
| 9911 | AIBV 3′ UTR |
| 9912-9913 | HOBMPRO, HOBHEGA nucleotide sequences for FIGS. 3B, 3C |
| 9914-9918 | Human CoV amino acid sequences for FIGS. 4A, 4B, 4C, 4E, 4F |
| 9919 | HCoV-OC43 5′ UTR |
| 9920 | HCoV-OC43 3′ UTR |
| 9921-9923 | pCMVKm2 vectors |
| 9924-9926 | Codon-optimised N, M and E sequences |
| 9927 | BNI-1 |
| 9928-9959 | Constituent amino acid sequences ≧4aa inferred from SEQ ID NO: 9927 |
| 9960 | ORF1ab variant |
| 9961 | ORF1a variant |
| 9962 | Spike variant |
| 9963 | Membrane variant |
| 9964 | Nucleocapsid variant |
| 9965-9966 | Short ORFs |
| 9967 | FRA complete genome |

TABLE 35

| Com- | | | | |
|---|---|---|---|---|
| pound | | | | |
| # | Structure | Source | Literature Reference | Patent Number |
| 1 | 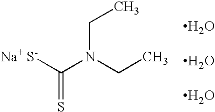 | Aventis Pasteur | 1) Lang. J. -M.; Touraine, J. -L.; Trepo, C, et al. Lancet 1988, 2(8613): 702-5. | |
| 2 | 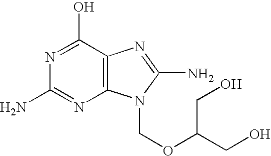 | Pfizer | Dong, M. K. et al. Pharmacologist 1988, 30(3): Abst 87.8. | ES 8602792 |
| 3 | 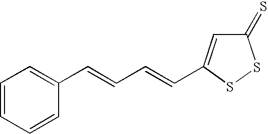 | Mitsui Chemicals | Mizuno, O. et al. 4th Int Conf Immunopharmacol (May 15-19, Osaka) 1988, Abst WS6-3. | EP 236929 |
| 4 | 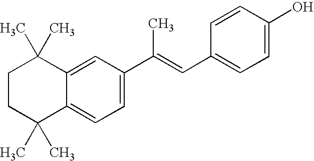 | Roche | | EP 407788 |
| 5 | 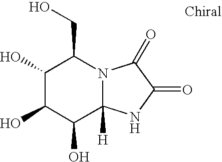 | Fujisawa | 1) Iwami, M. et al. J Antibiot 1987, 40(5): 612-22. | JP 87161796 |
| 6 | 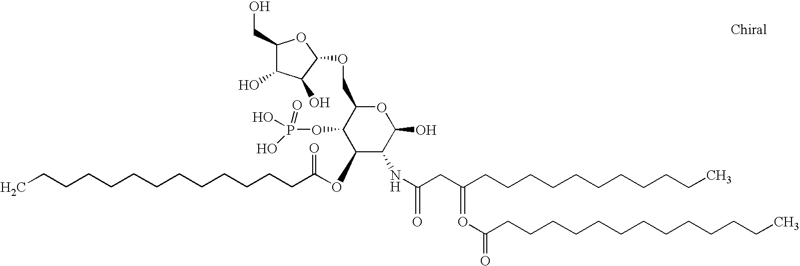 | Novartis | | FR 2604177 |
| 7 | 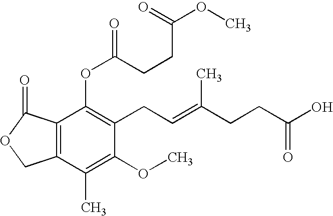 | Roche Bioscience | | US 4725622 |
| 8 | 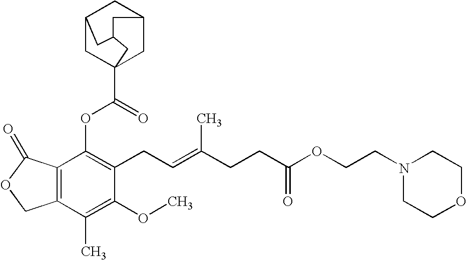 | Roche Bioscience | | US 4727069 |
| 9 | 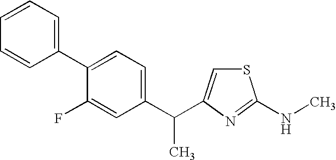 | Sumitomo | 1) Nishikaku, F. and Koga. Y. 4th Int Conf Immunopharmacol (May 15-19, Osaka) 1988, Abst WS6-8. | EP 248399 |
| 10 | 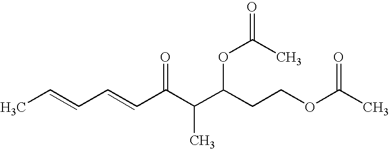 | SSP | | JP 88022053 |
| 11 | 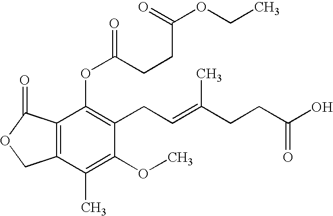 | Roche Bioscience | | US 4725622 |
| 12 | 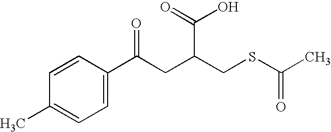 | Taisho | 1) Kameo, K. et al. Chem Pharm Bull 1988, 36(6): 2050-60. | EP 164101 |
| 13 | 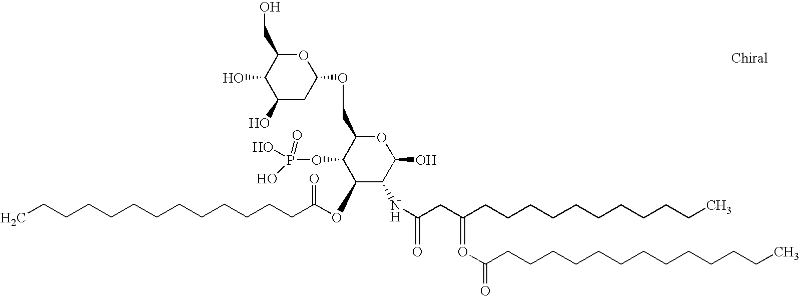 | Novartis | | FR 2604177 |
| 14 | 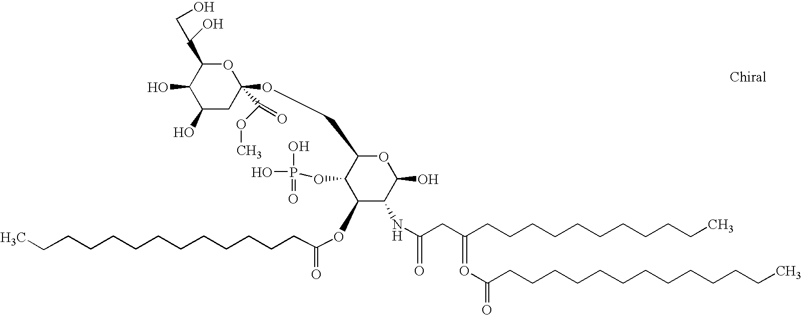 | Novartis | | FR 2604177 |
| 15 | 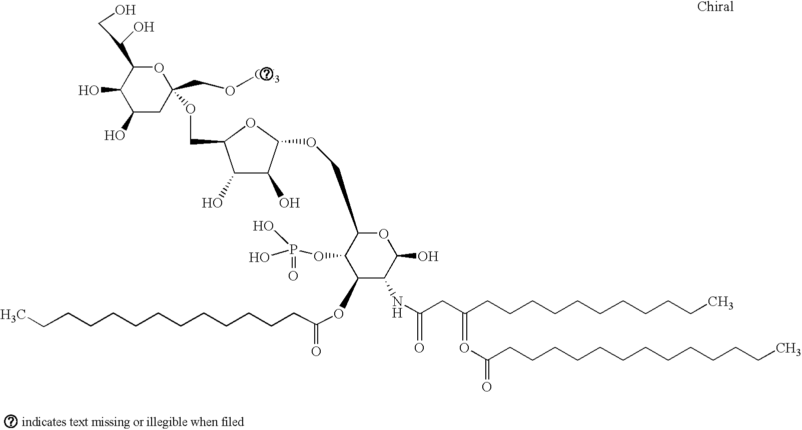 | Novartis | | FR 2604177 |
| 16 | 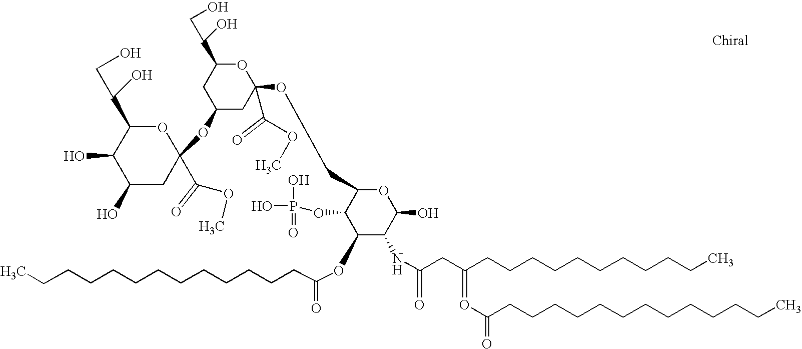 | Novartis | | FR 2604177 |
| 17 | 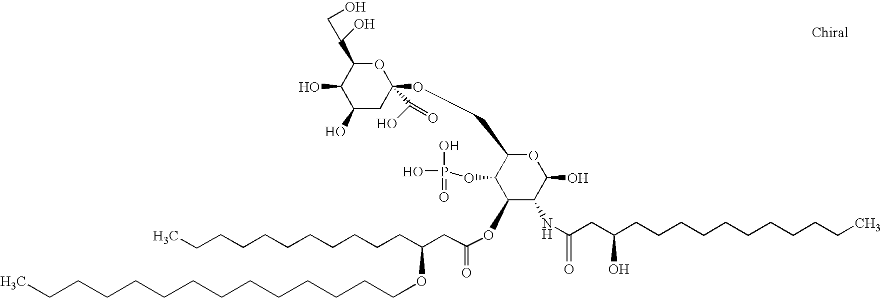 | Novartis | | FR 2604177 |
| 18 | 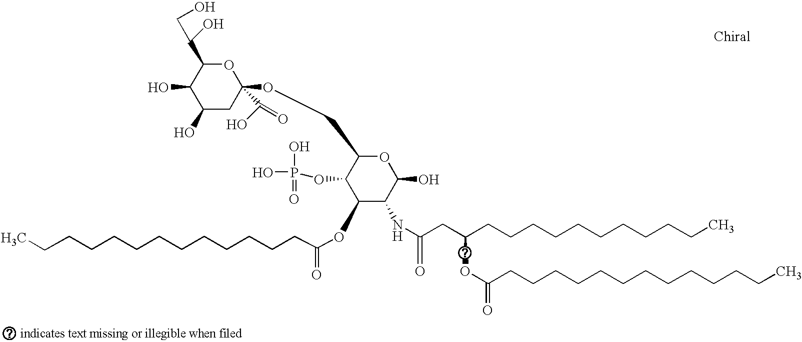 | Novartis | | FR 2604177 |
| 19 | 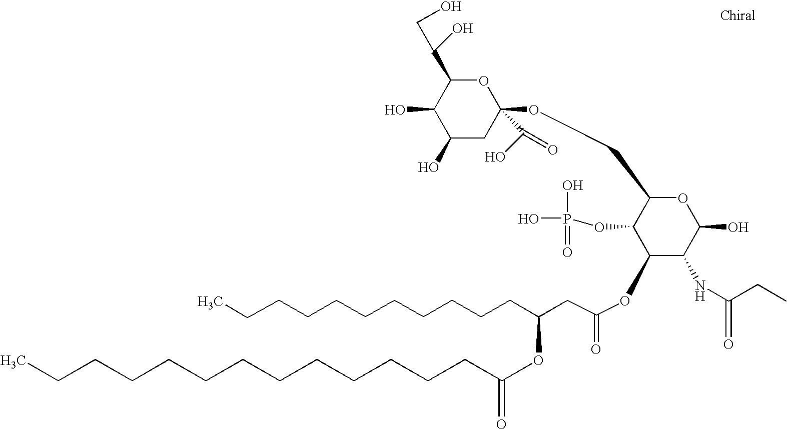 | Novartis | | FR 2604177 |
| | 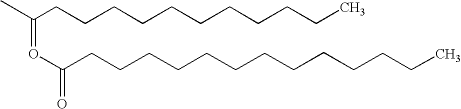 | | | |
| 20 | 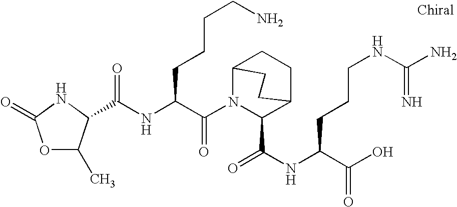 | ADIR | | AU 8811669 |
| 21 | 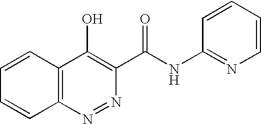 | Pharmacia | | AU 8810908 |
| 22 | 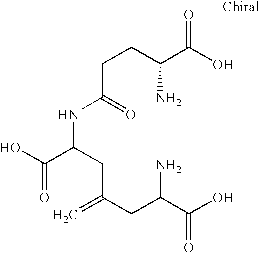 | Aventis Pharma | | EP 284461 |
| 23 | 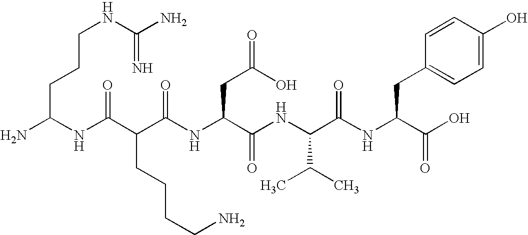 | Eniricerche | | EP 282891 |
| 24 | 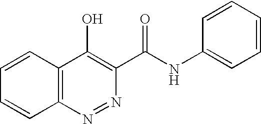 | Pharmacia | | AU 8810908 |
| 25 | 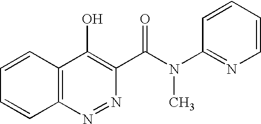 | Pharmacia | | AU 8810908 |
| 26 | 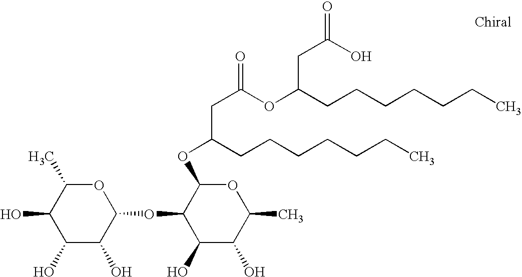 | Abbott | Swanson, R. N. et at. 28th Intersci Conf Antimicrob Agents Chemother (Oct 23-26, Los Angeles) 1988, Abst 972. | |
| 27 | 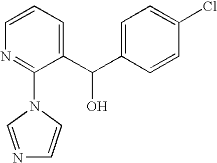 | Mitsubishi Pharma | | JP 88119425 |
| 28 | 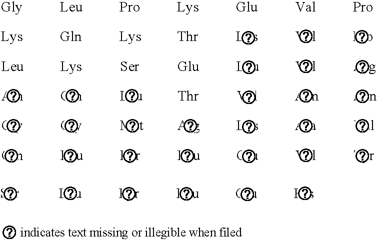 | Ortho-McNeil | | EP 292302 |
| 29 | 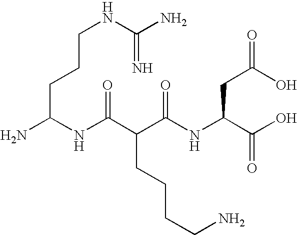 | Eniricerche | | EP 282891 |
| 30 | 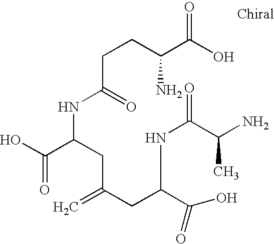 | Aventis Pharma | | EP 284461 |
| 31 | 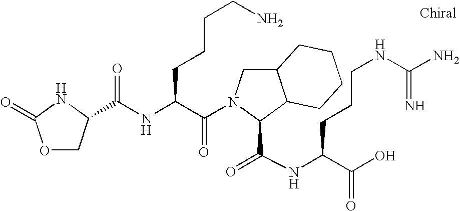 | ADIR | | AU 8811669 |
| 32 | 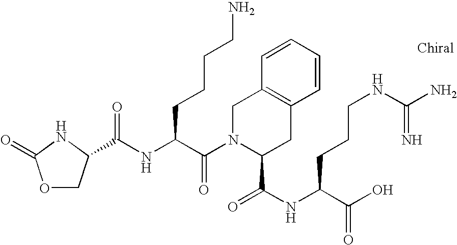 | ADIR | | AU 8811669 |
| 33 | 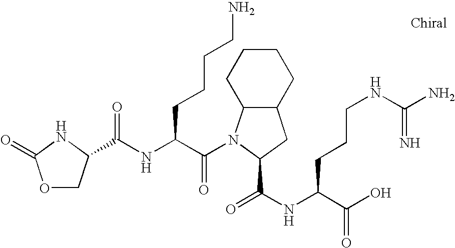 | ADIR | | AU 8811669 |
| 34 | 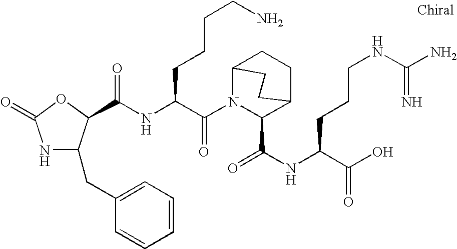 | ADIR | | AU 8811669 |
| 35 | 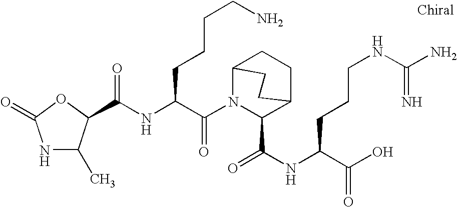 | ADIR | | AU 8811669 |
| 36 | 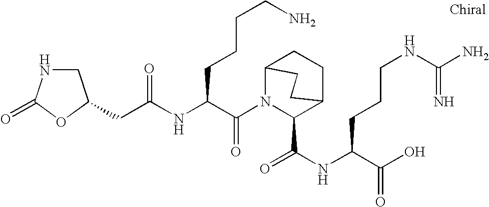 | ADIR | | AU 8811669 |
| 37 | 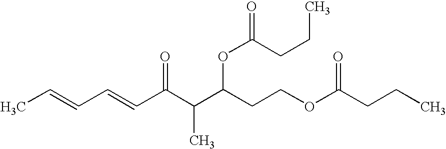 | SSP | | JP 88022053 |
| 38 | 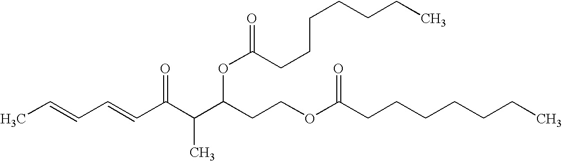 | SSP | | JP 88022053 |
| 39 | 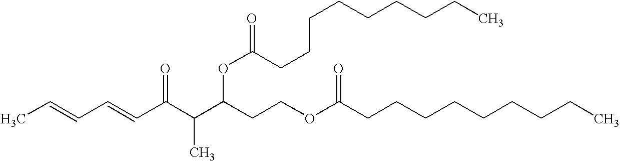 | SSP | | JP 88022053 |
| 40 | 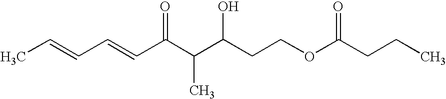 | SSP | | JP 88022053 |
| 41 | 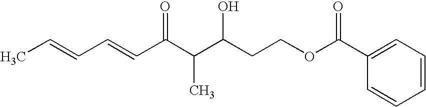 | SSP | | JP 88022053 |
| 42 | 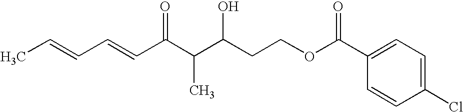 | SSP | | JP 88022053 |
| 43 | 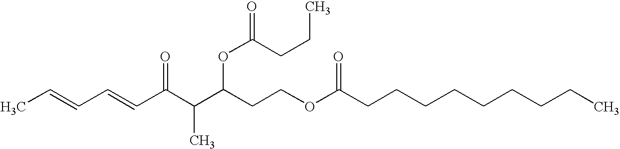 | SSP | | JP 88022053 |
| 44 | 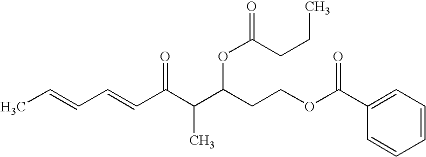 | SSP | | JP 88022053 |
| 45 | 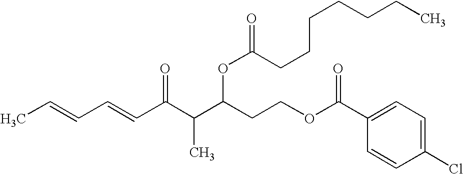 | SSP | | JP 88022053 |
| 46 | 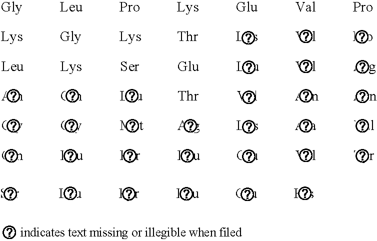 | Ortho-McNeil | | EP 300741 |
| 47 | 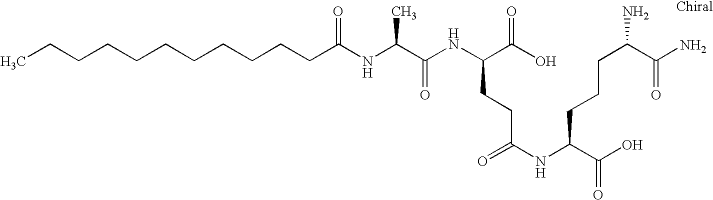 | Aventis Pharma | 1) Fizames, C. et al. 15th Int Cong Chemother (July 19-24, Istanbul) 1987, Abst 605 | 1) GB 2053231 |
| 48 | 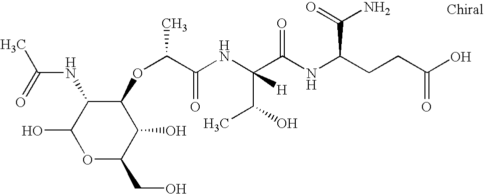 | Roche Bioscience | 1) WHO Drug Inform 1988, 2(4): 227. | 1) EP 135376 |
| 49 | 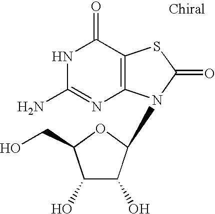 | ICN | 1) Sharma, B. S. et at. 3rd Intersci World Conf Inflamm, Antirheum Anaig Immunomodul (March 15-18, Monte- Carlo) 1989, 9. | EP 348446 |
| 50 | 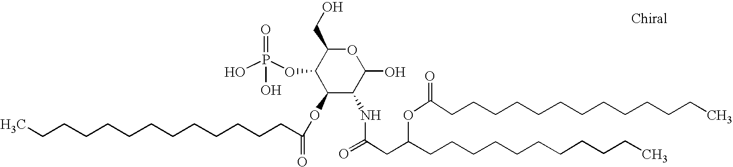 | Toho Yakuhin | 1) Satoru, I. et al. Antivir Res 1988, 9(1-2): 37-46. | EP 188697 |
| 51 | 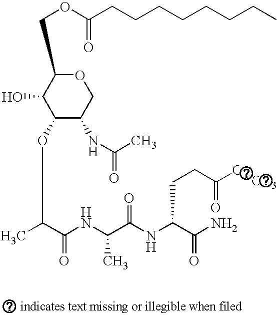 | Gifu University | 1) Hasegawa, A. et al. J Carbohyd Chem 1986, 5(3): 371-85. | |
| | 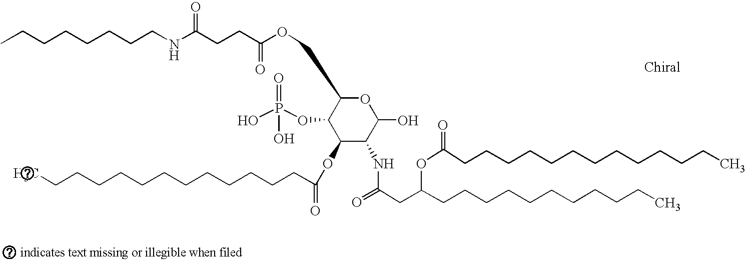 | | | |
| 52 | 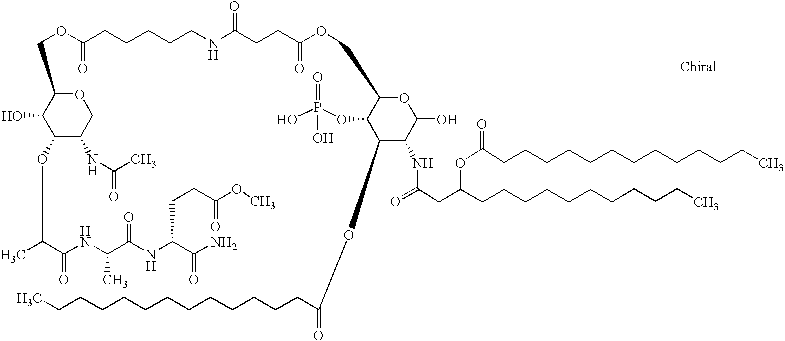 | Gifu University | | |
| 53 | 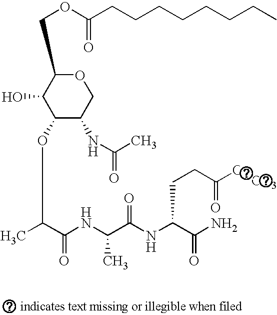 | Gifu University | | |
| | 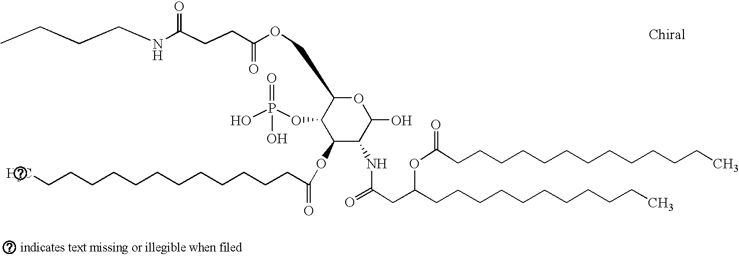 | | | |
| 54 | 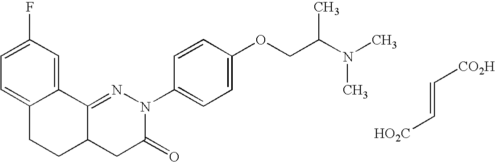 | Mitsubishi Pharma | | EP 351435 |
| 55 | 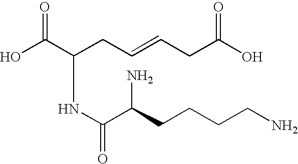 | Aventis Pharma | | AU 8824582 |
| 56 | 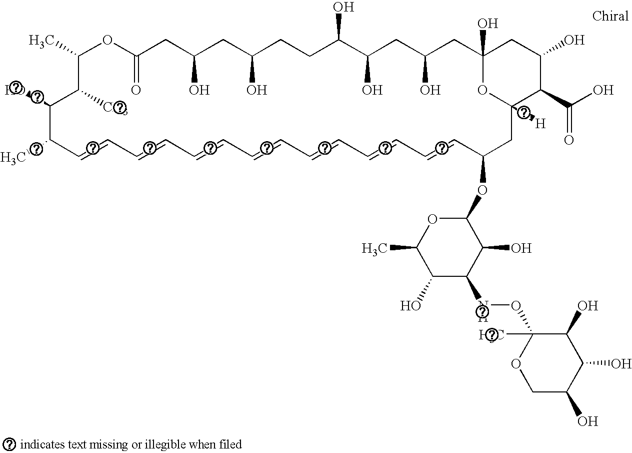 | CNRS | 1) Chiron, M. et al. Biochem Pharmacol 1988, 37(5): 827-36. | |
| 57 | 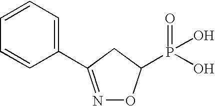 | Aventis Pharma | 1) Schorlemmer, H. U.; Bartlett, R. R.; Dickneite, G.; Schwab, W.; Gebert, U.; Sedlacek, H. H. | AU 8824195 |
| 58 | 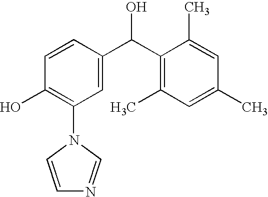 | Mitsubishi Pharma | | JP 88119425 |
| 59 | 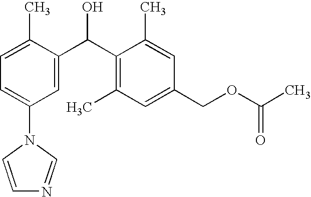 | Mitsubishi Pharma | | JP 88119425 |
| 60 | 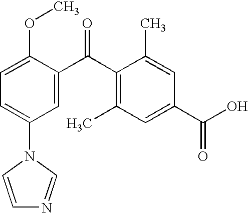 | Mitsubishi Pharma | | JP 88119425 |
| 61 | 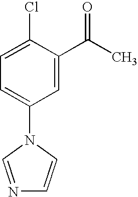 | Mitsubishi Pharma | | JP 88119425 |
| 62 | 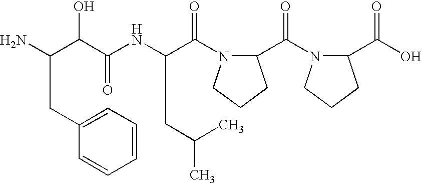 | Microbial Chemistry Research Foundation | | EP 310238 |
| 63 | 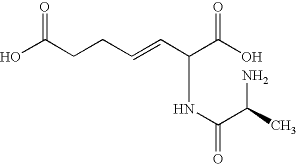 | Aventis Pharma | | AU 8824582 |
| 64 | 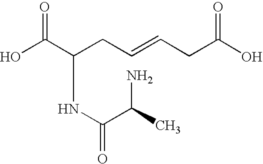 | Aventis Pharma | | AU 8824582 |
| 65 | 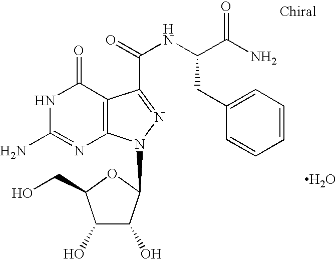 | ICN | Ramasamy, K. et al. J Med Chem 1989, 32(8): 1905-9. | |
| 66 | 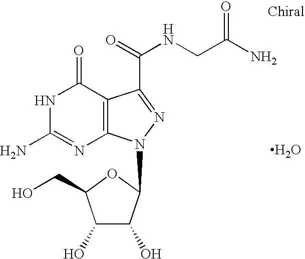 | ICN | | |
| 67 | 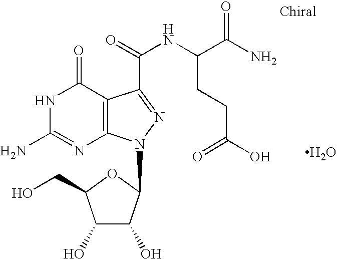 | ICN | | |
| 68 | 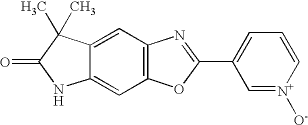 | Roche | | AU 8931653 |
| 69 | 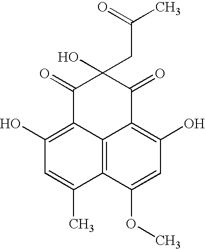 | Fujisawa | Shibata, T. et at. J Antibiot 1989, 42(9): 1356-61. | |
| 70 | 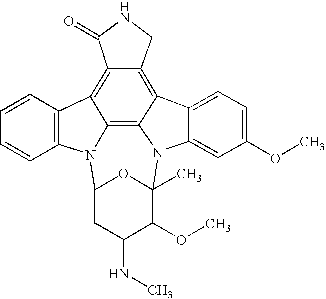 | Takeda | Tanida, S. et al. J Antibiot 1989, 42(11): 1619. | |
| 71 | 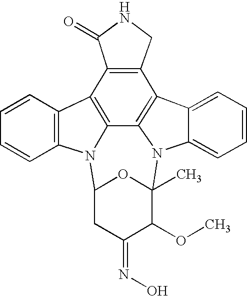 | Takeda | | |
| 72 | 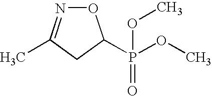 | Aventis Pharma | | AU 8824195 |
| 73 | 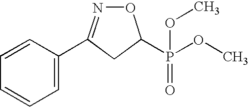 | Aventis Pharma | | AU 8824195 |
| 74 | 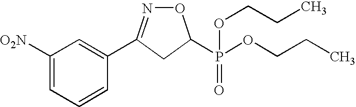 | Aventis Pharma | | AU 8824195 |
| 75 | 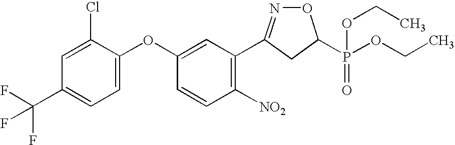 | Aventis Pharma | | AU 8824195 |
| 76 | 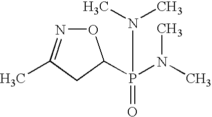 | Aventis Pharma | | AU 8824195 |
| 77 | 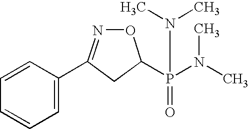 | Aventis Pharma | | AU 8824195 |
| 78 | 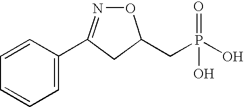 | Aventis Pharma | | AU 8824195 |
| 79 | 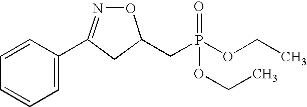 | Aventis Pharma | | AU 8824195 |
| 80 | 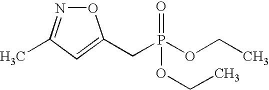 | Aventis Pharma | | AU 8824195 |
| 81 | 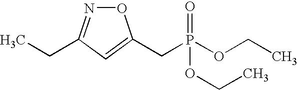 | Aventis Pharma | | AU 8824195 |
| 82 | 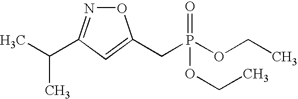 | Aventis Pharma | | AU 8824195 |
| 83 | 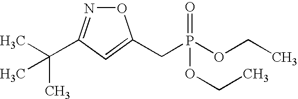 | Aventis Pharma | | AU 8824195 |
| 84 | 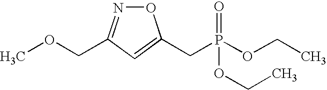 | Aventis Pharma | | AU 8824195 |
| 85 | 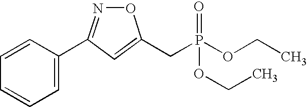 | Aventis Pharma | | AU 8824195 |
| 86 | 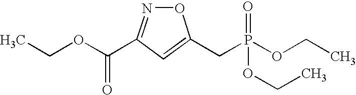 | Aventis Pharma | | AU 8824195 |
| 87 | 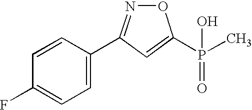 | Aventis Pharma | | AU 8824195 |
| 88 | 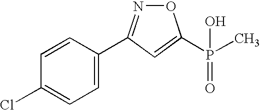 | Aventis Pharma | | AU 8824195 |
| 89 | 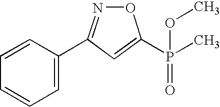 | Aventis Pharma | | AU 8824195 |
| 90 | 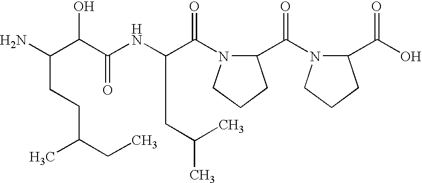 | Microbial Chemistry Research Foundation | | EP 310238 |
| 91 | 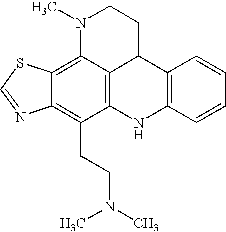 | Harbor Branch Oceanographic Institution | | EP 331320 |
| 92 | 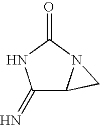 | Roche | 1) Herrmann. D. B. J. 17th Int Cong Chemother (June 23-28, Berlin) 1991, Abst 838. | EP 352652 |
| 93 | 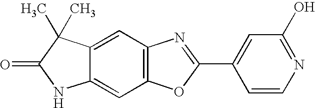 | Roche | | AU 8931653 |
| 94 | 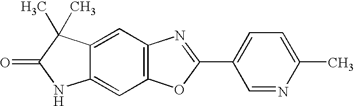 | Roche | | AU 8931653 |
| 95 | 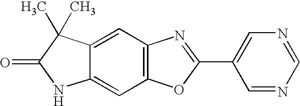 | Roche | | AU 8931653 |
| 96 | 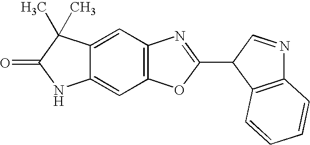 | Roche | | AU 8931653 |
| 97 | 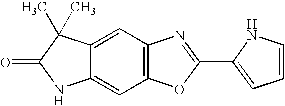 | Roche | | AU 8931653 |
| 98 | 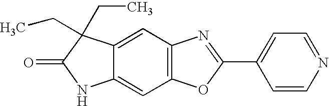 | Roche | | AU 8931653 |
| 99 | 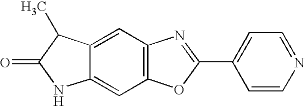 | Roche | | AU 8931653 |
| 100 | 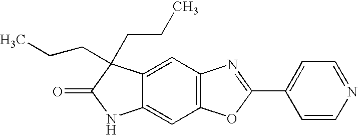 | Roche | | AU 8931653 |
| 101 | 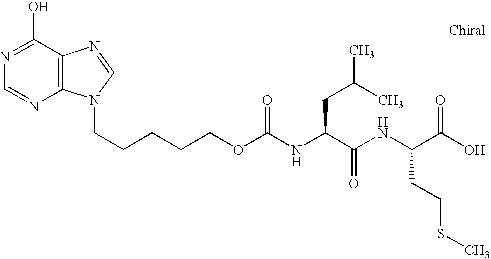 | Scharper | 1) Migliorati. G. et al. 7th Int Cong Immunol (July 30-Aug 5, Berlin) 1989, Abst 106-62. | |
| 102 | 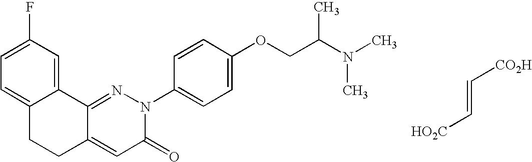 | Mitsubishi Pharma | | EP 351435 |
| 103 | 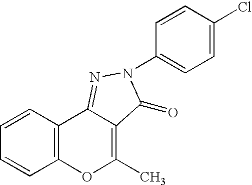 | Abbott GmbH | | EP 354693 |
| 104 | 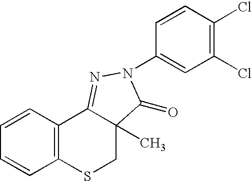 | Abbott GmbH | | EP 354694 |
| 105 | 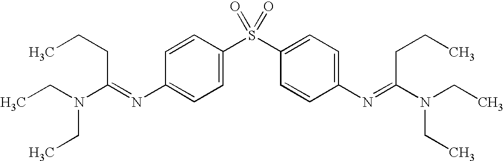 | Wyeth | | EP 354303 |
| 106 | 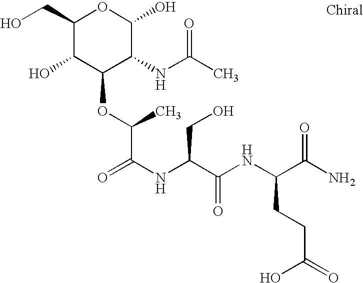 | Merck & Co. | | US 4866035 |
| 107 | 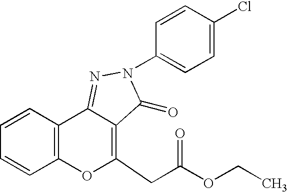 | Abbott GmbH | | EP 354693 |
| 108 | 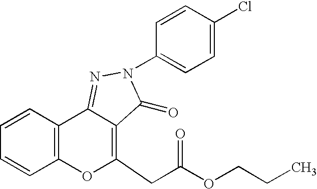 | Abbott GmbH | | EP 354693 |
| 109 | 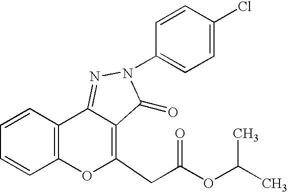 | Abbott GmbH | | EP 354693 |
| 110 | 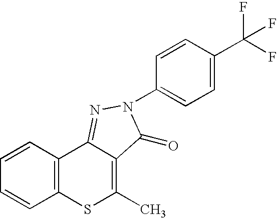 | Abbott GmbH | | EP 354694 |
| 111 | 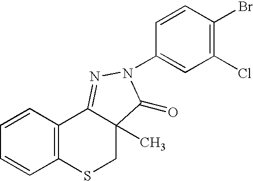 | Abbott GmbH | | EP354694 |
| 112 | 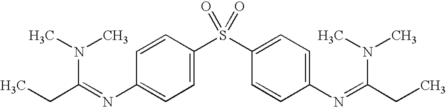 | Wyeth | | EP 354303 |
| 113 | 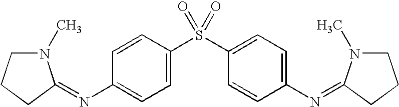 | Wyeth | | EP 354303 |
| 114 | 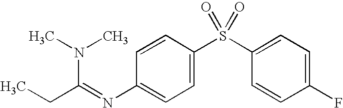 | Wyeth | | EP 354303 |
| 115 | 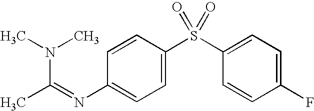 | Wyeth | | EP 354303 |
| 116 | 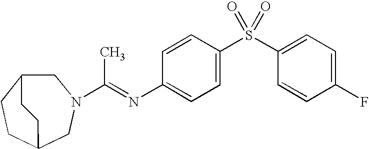 | Wyeth | | EP 354303 |
| 117 | 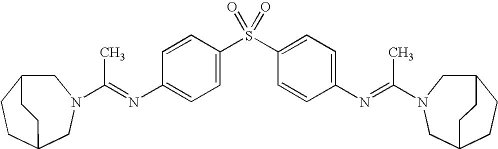 | Wyeth | | EP 354303 |
| 118 | 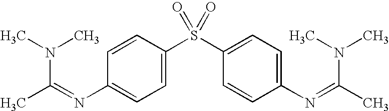 | Wyeth | | EP 354303 |
| 119 | 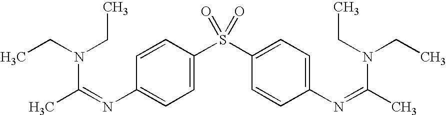 | Wyeth | | EP 354303 |
| 120 | 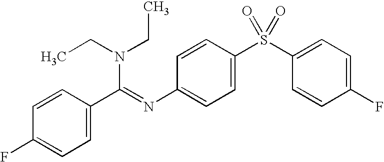 | Wyeth | | EP 354303 |
| 121 | 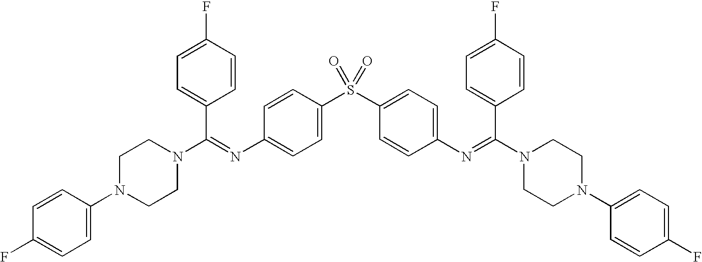 | Wyeth | | EP 354303 |
| 122 | 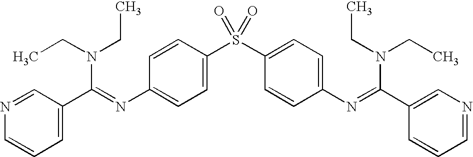 | Wyeth | | EP 354303 |
| 123 | 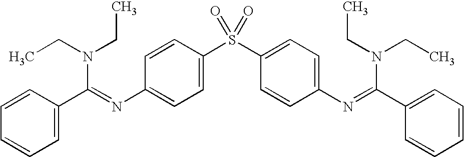 | Wyeth | | EP 354303 |
| 124 | 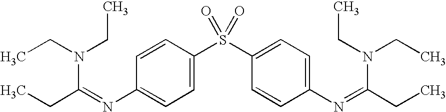 | Wyeth | | EP 354303 |
| 125 | 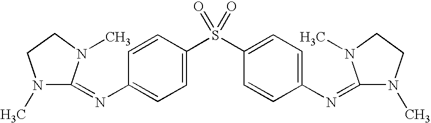 | Wyeth | | EP 354303 |
| 126 | 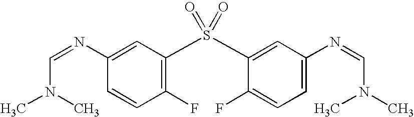 | Wyeth | | EP 354303 |
| 127 | 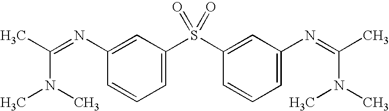 | Wyeth | | EP 354303 |
| 128 | 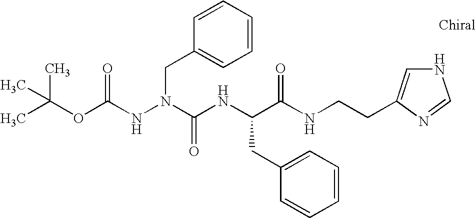 | Tanabe | | EP 372818 |
| 129 | 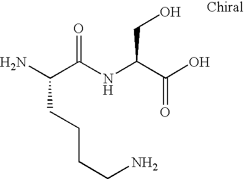 | Elan | 1) Eldon, M. A. et al. J Olin Pharmacol 1990, 30(4): 352-7. | |
| 130 | 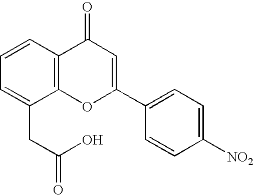 | Lipha | | EP 341104 |
| 131 | 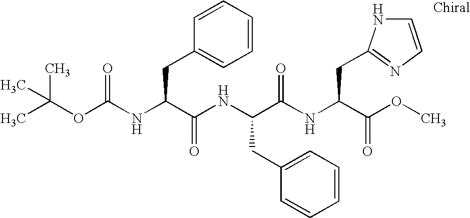 | Tanabe | | EP 372818 |
| 132 | 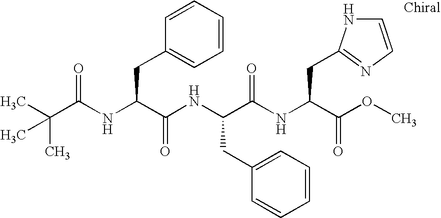 | Tanabe | | EP 372818 |
| 133 | 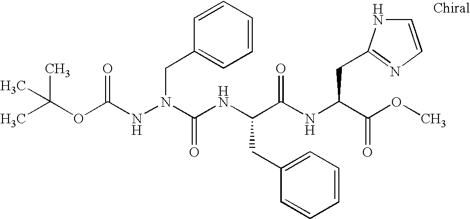 | Tanabe | | EP 372818 |
| 134 | 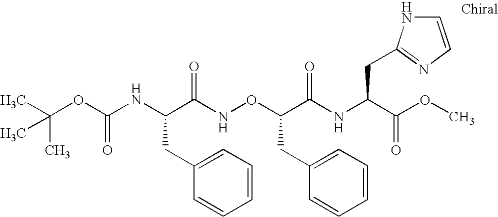 | Tanabe | | EP 372818 |
| 135 | 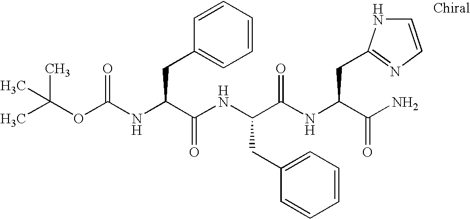 | Tanabe | | EP 372818 |
| 136 | 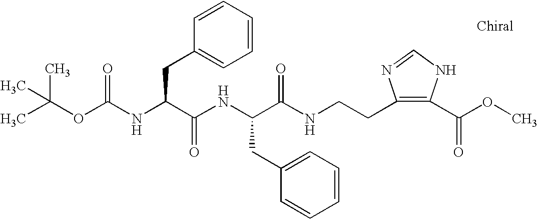 | Tanabe | | EP 372818 |
| 137 | 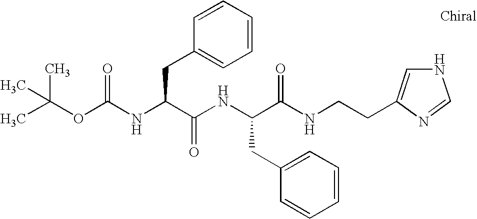 | Tanabe | | EP 372818 |
| 138 | 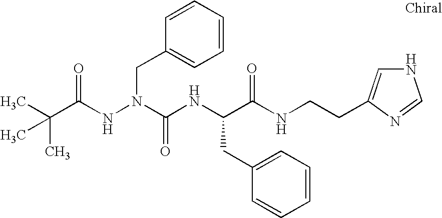 | Tanabe | | EP 372818 |
| 139 | 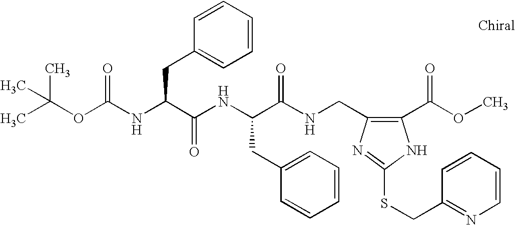 | Tanabe | | EP 372818 |
| 140 | 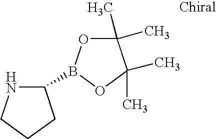 | Bristol-Myers Squibb | | US 4935493 |
| 141 | 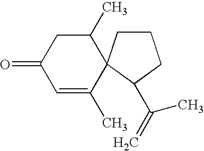 | Takeda | | JP 90193940 |
| 142 | 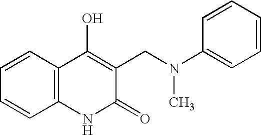 | Pharmacia | | EP 429627 |
| 143 | 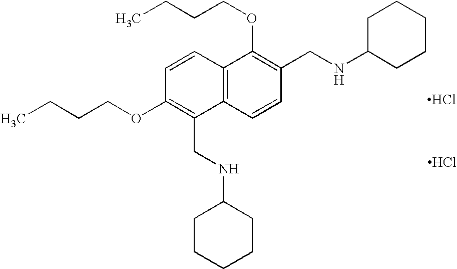 | Merck & Co. | | EP 393256 |
| 144 | 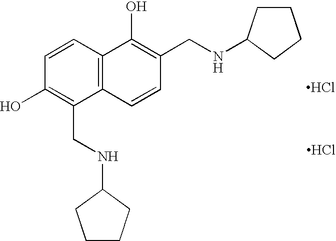 | Merck & Co. | | EP 393256 |
| 145 | 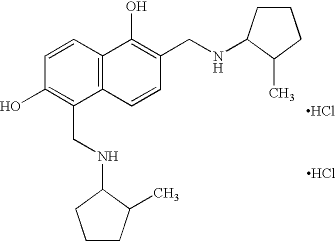 | Merck & Co. | | EP 393256 |
| 146 | 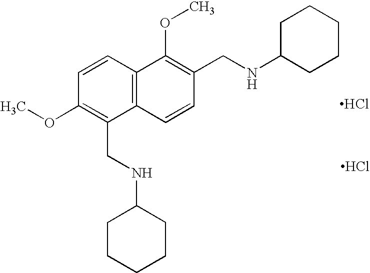 | Merck & Co. | | EP 393256 |
| 147 | 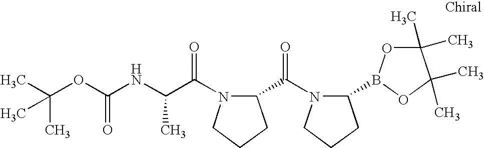 | Bristol-Myers Squibb | | US 4935493 |
| 148 | 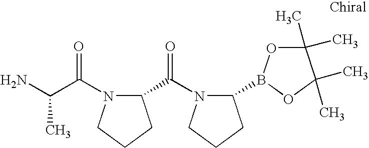 | Bristol-Myers Squibb | | US 4935493 |
| 149 | 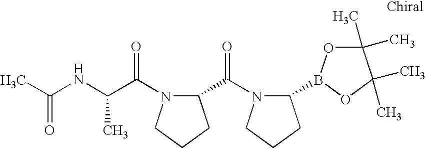 | Bristol-Myers Squibb | | US 4935493 |
| 150 | 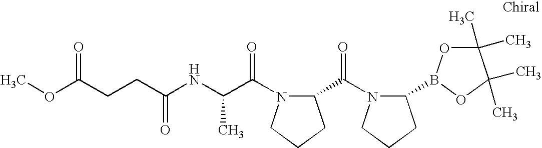 | Bristol-Myers Squibb | | US 4935493 |
| 151 | 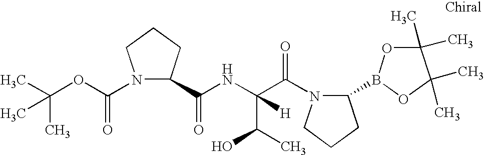 | Bristol-Myers Squibb | | US 4935493 |
| 152 | 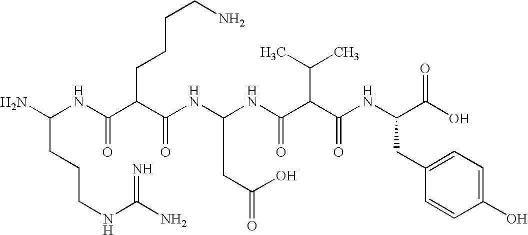 | Sclavo | | EP 375058 |
| 153 | 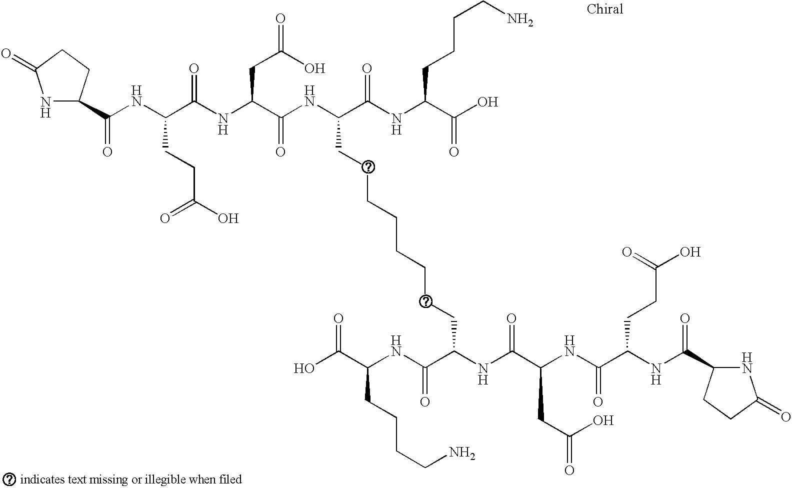 | SmithKline Beecham | | AU 9059014 |
| 154 | 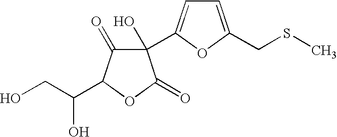 | American Biotechnology | | WO 9102733 |
| 155 | 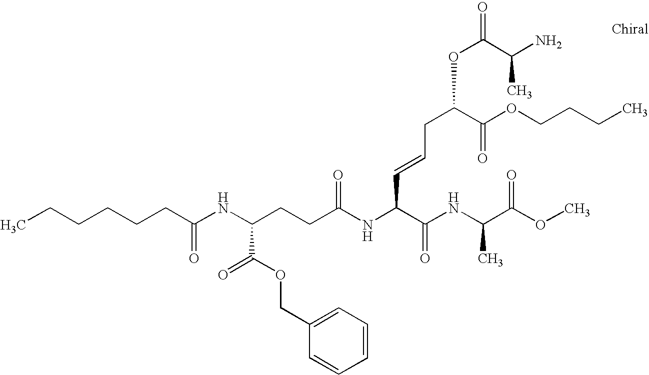 | Novartis | | EP 417803 |
| 156 | 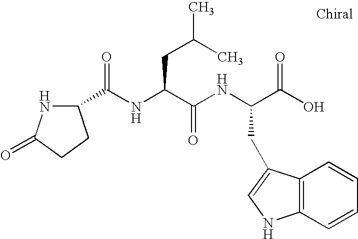 | Polifarma | | EP 423077 |
| 157 | 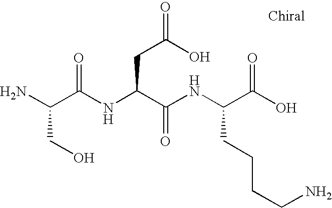 | SCRAS | | GB 2228937 |
| 158 | 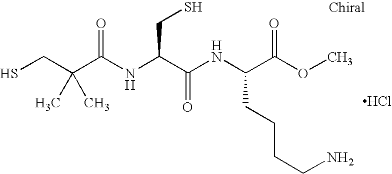 | Santen | | EP 455833 |
| 159 | 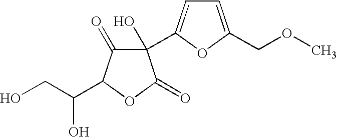 | American Biotechnology | | WO 9102733 |
| 160 | 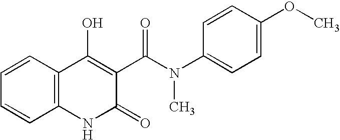 | Pharmacia | | EP 429627 |
| 161 | 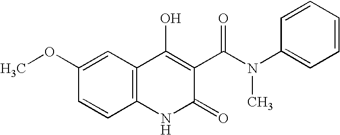 | Pharmacia | | EP 429627 |
| 162 | 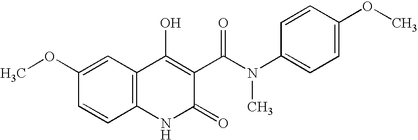 | Pharmacia | | EP 429627 |
| 163 | 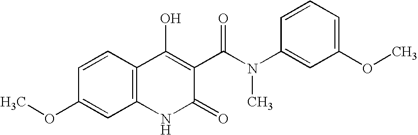 | Pharmacia | | EP 429627 |
| 164 | 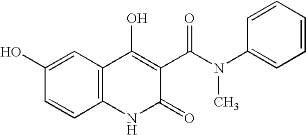 | Pharmacia | | EP 429627 |
| 165 | 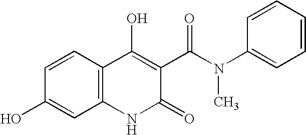 | Pharmacia | | EP 429627 |
| 166 | 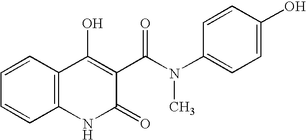 | Pharmacia | | EP 429627 |
| 167 | 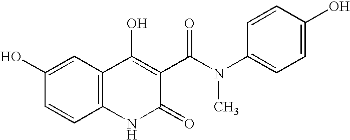 | Pharmacia | | EP 429627 |
| 168 | 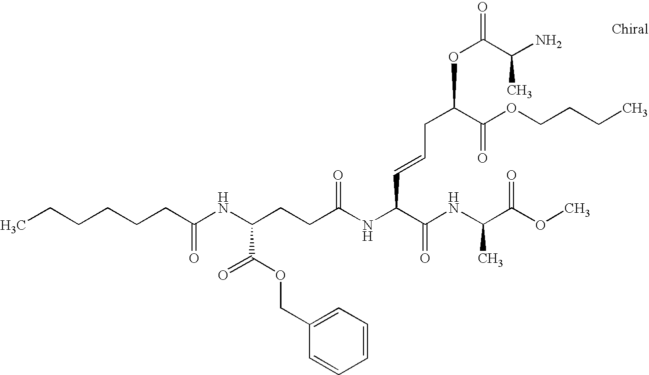 | Novartis | | EP 417803 |
| 169 | 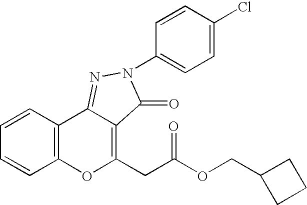 | Abbott GmbH | | WO 9111448 |
| 170 | 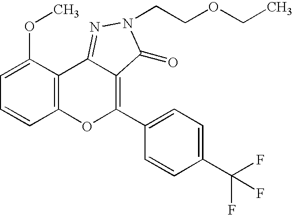 | Abbott GmbH | | WO 9112255 |
| 171 | 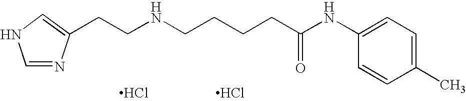 | Leland Stanford Junior University | | US 4996221 |
| 172 | 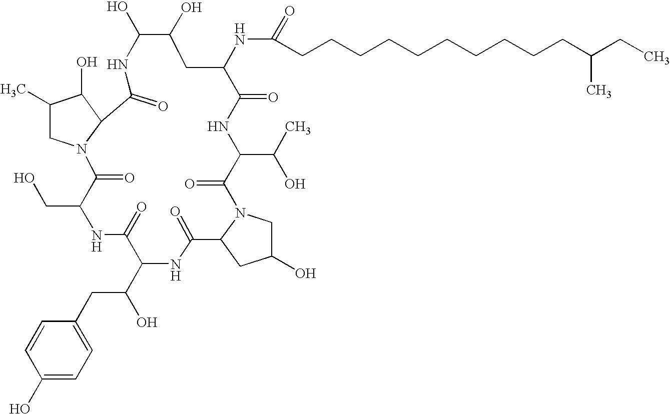 | Aventis Pharma | | EP 438813 |
| 173 | 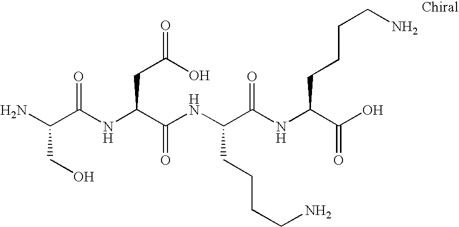 | SCRAS | | GB 2228937 |
| 174 | 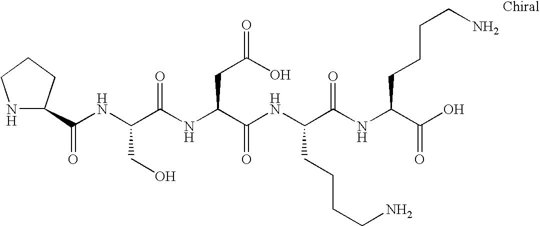 | SCRAS | | GB 2228937 |
| 175 | 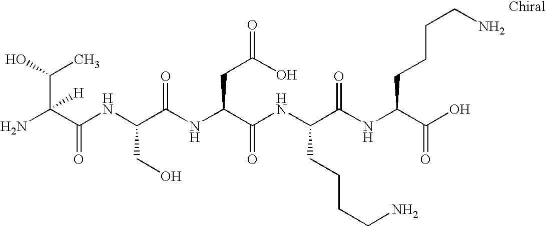 | SCRAS | | GB 2228937 |
| 176 | 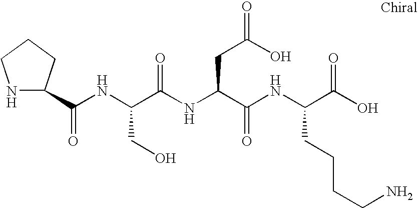 | SCRAS | | GB 2228937 |
| 177 | 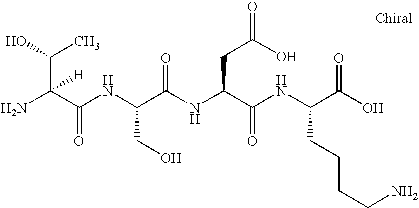 | SCRAS | | GB 2228937 |
| 178 | 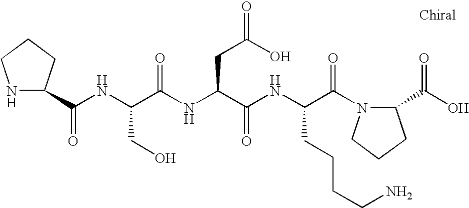 | SCRAS | | GB 2228937 |
| 179 | 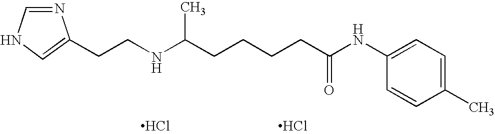 | Leland Stanford Junior University | | US 4996221 |
| 180 | 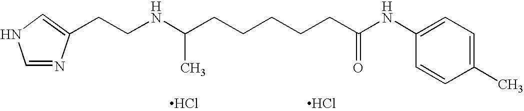 | Leland Stanford Junior University | | US 4996221 |
| 181 | 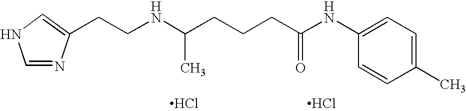 | Leland Stanford Junior University | | US 4996221 |
| 182 | 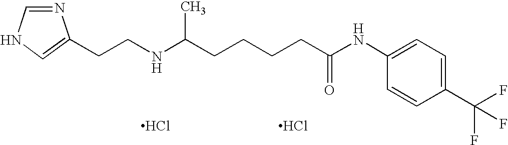 | Leland Stanford Junior University | | US 4996221 |
| 183 | 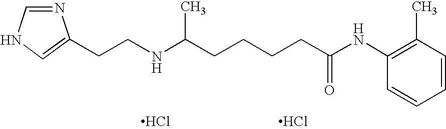 | Leland Stanford Junior University | | US 4996221 |
| 184 | 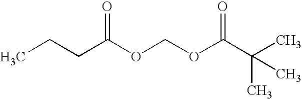 | Bar-llan University | 1) Rephaeli, A. et al. Int J Cancer 1991, 49(1): 66. | EP 302349 |
| 185 | 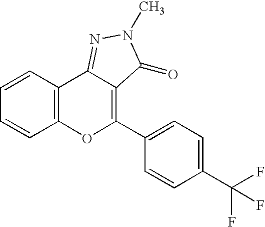 | Abbott GmbH | | WO 9112255 |
| 186 | 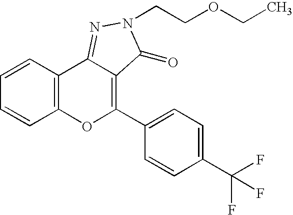 | Abbott GmbH | | WO 9112255 |
| 187 | 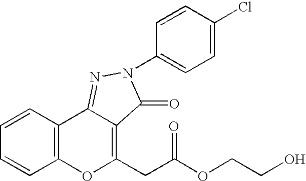 | Abbott GmbH | | WO 9111448 |
| 188 | 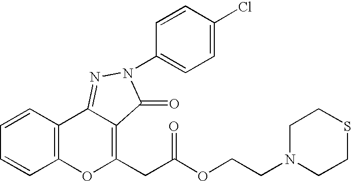 | Abbott GmbH | | WO 9111448 |
| 189 | 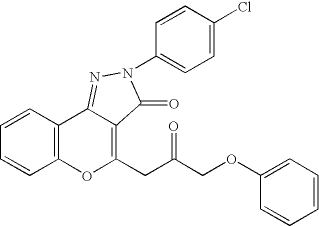 | Abbott GmbH | | WO 9111448 |
| 190 | 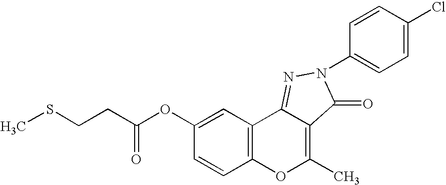 | Abbott GmbH | | WO 9111448 |
| 191 | 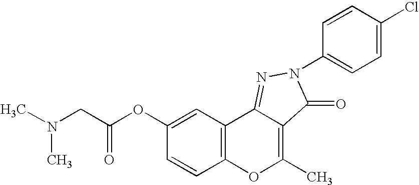 | Abbott GmbH | | WO 9111448 |
| 192 | 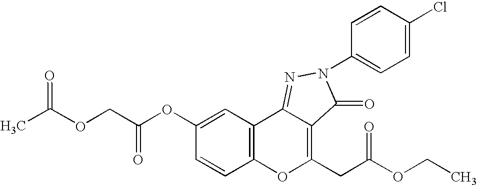 | Abbott GmbH | | WO 9111448 |
| 193 | 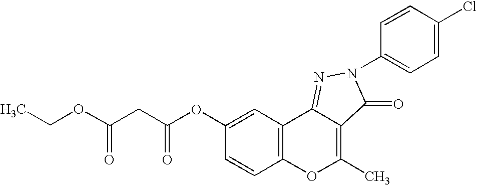 | Abbott GmbH | | WO 9111448 |
| 194 | 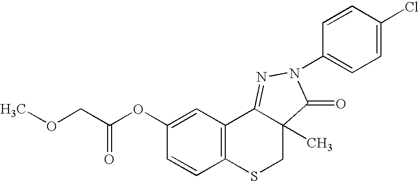 | Abbott GmbH | | WO 9111448 |
| 195 | 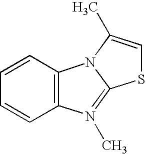 | Bashkir Medical University | Dianov, V. M. et al. Khim Farm Zh SSSR 1991, 25(1): 40. | |
| 196 | 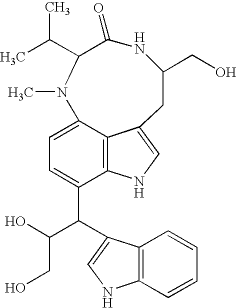 | Microbial Chemistry Research Foundation | Kumagai, H. et al. J Antibiot 1991, 44(9): 1029. | |
| 197 | 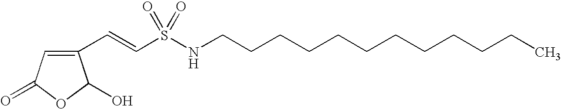 | Allergan | | US 5081261 |
| 198 | 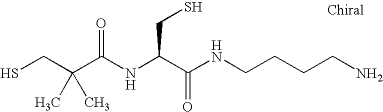 | Santen | | EP 455833 |
| 199 | 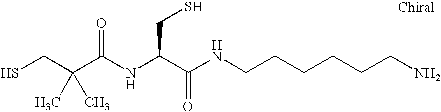 | Santen | | EP 455833 |
| 200 | 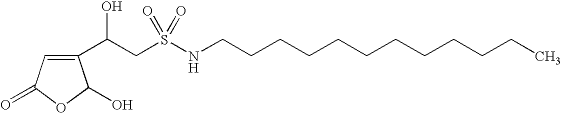 | Allergan | | US 5081261 |
| 201 | 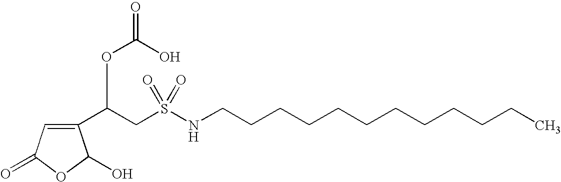 | Allergan | | US 5081261 |
| 202 | 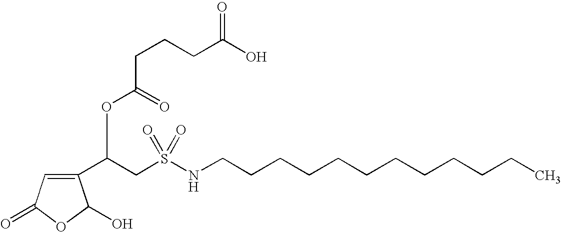 | Allergan | | US 5081261 |
| 203 | 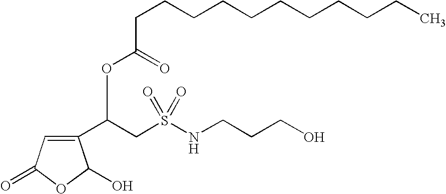 | Allergan | | US 5081261 |
| 204 | 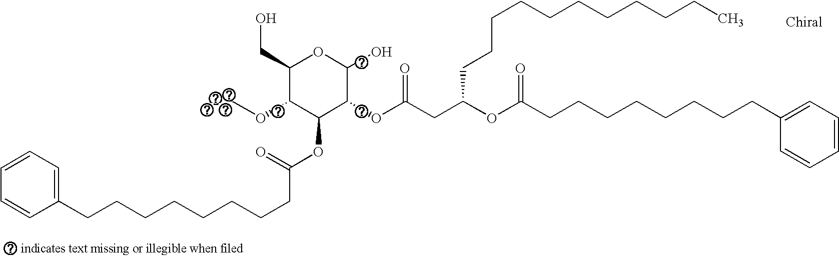 | Ono | 1) Satoh, M. et al. 51st Annu Meet Jpn Cancer Assoc (Sept 29-Oct 1. Osaka) 1992. Abst 1450 | EP 553786 |
| 205 | 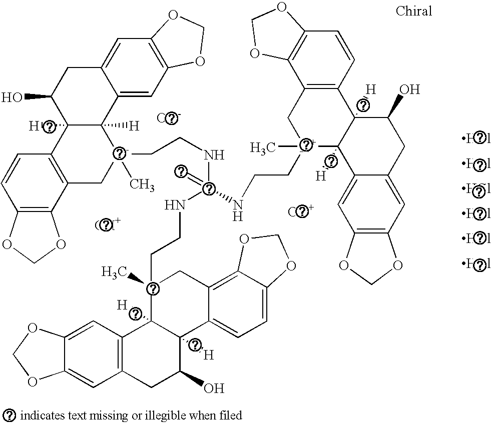 | Nowicky | 1) Hohenwarter, O.; Strutzenberger, K.; Katinger, H.; Liepins, A.; Nowicky, J. W. Drug Exp Clin Res 1992, 18(Suppl.): 1. | WO 8300486 |
| 206 | 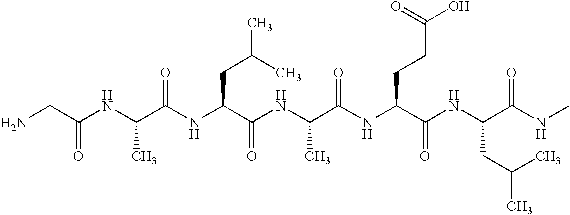 | Tanabe | | US 5210075 |
| | 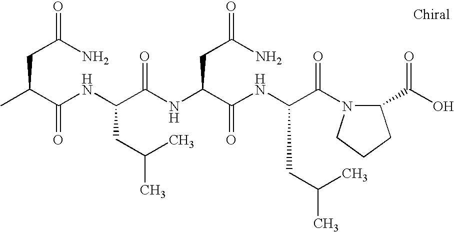 | | | |
| 207 | 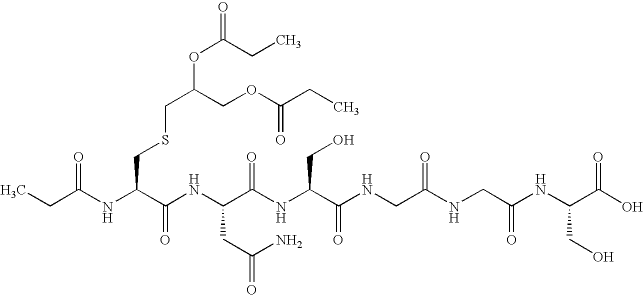 | Fujisawa | 1) Kurimura, M. et al. Pept Chem (1991) 1992, 361. | |
| 208 | 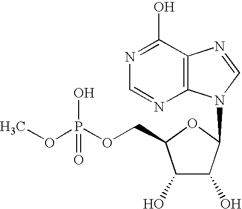 | University of South Florida | 1) Hadden, J. W. 5th Intersci World Conf Inflamm, Antirheum, Analg, Immunomodul (April 25-28, Geneva) 1993, Abst 257. | |
| 209 | 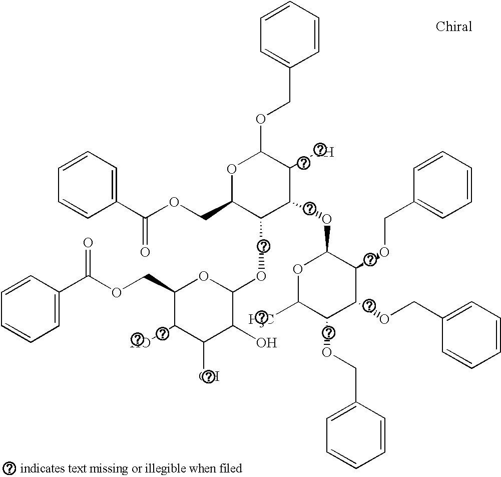 | Glycomed | | WO 9310796 |
| 210 | 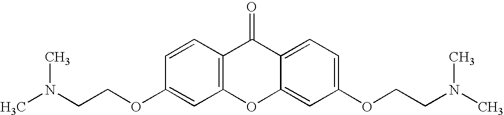 | Karolinska Institute | 1) Algarra, I. et al. Int J Cancer 1993, 54: 518. | |
| 211 | 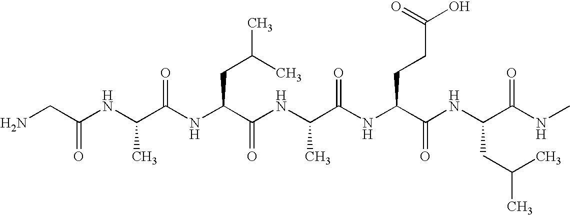 | Tanabe | | US 5210075 |
| | 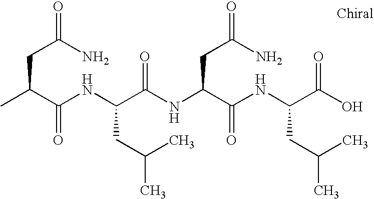 | | | |
| 212 | 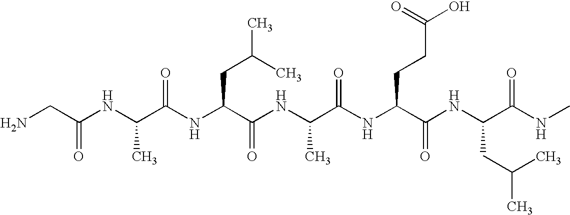 | Tanabe | | US 5210075 |
| | 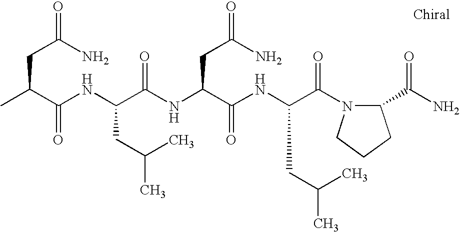 | | | |
| 213 | 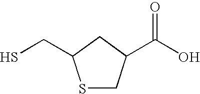 | Santen | | EP 558287 |
| 214 | 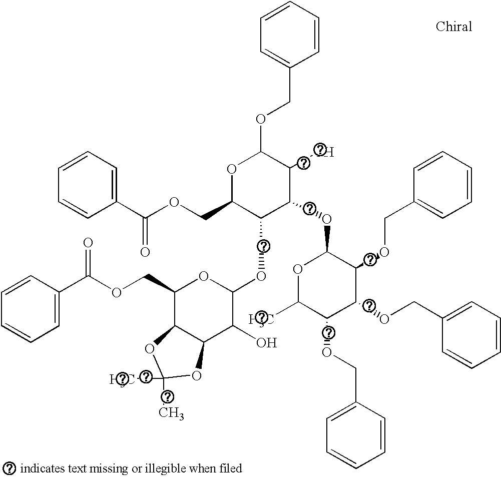 | Glycomed | | WO 9310796 |
| 215 | 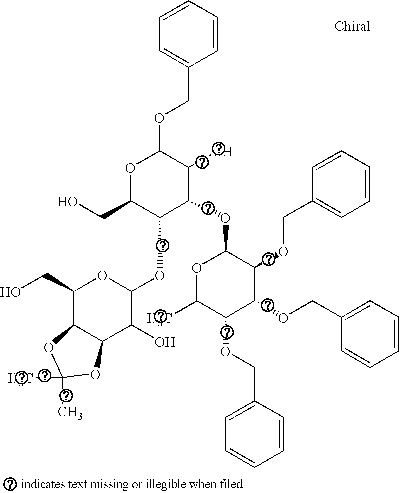 | Glycomed | | WO 9310796 |
| 216 | 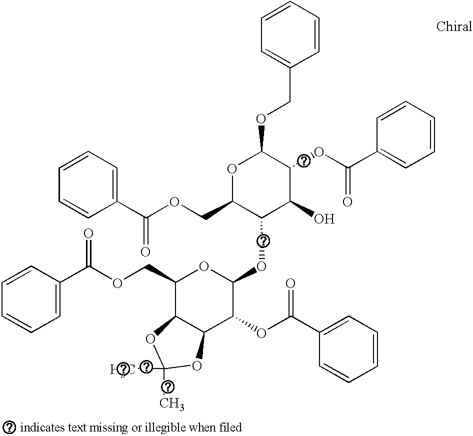 | Glycomed | | WO 9310796 |
| 217 | 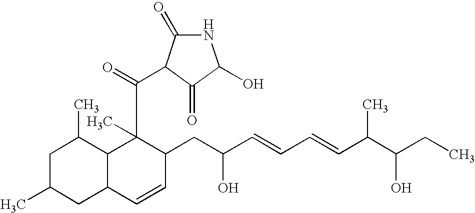 | Microbial Chemistry Research Foundation | | |
| 218 | 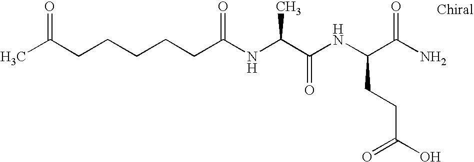 | LEK | 1) Sersa, G. et al. Mol Biother 1992, 4: 188. | EP 477912 |
| 219 | 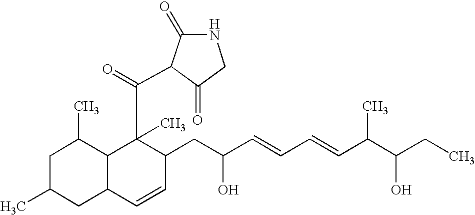 | Microbial Chemistry Research Foundation | 1) Ueno, M. et al. J Antibiot 1993. 46(5): 719. | |
| 220 | 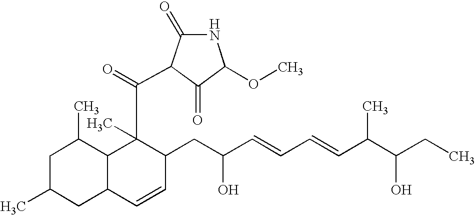 | Microbial Chemistry Research Foundation | | |
| 221 | 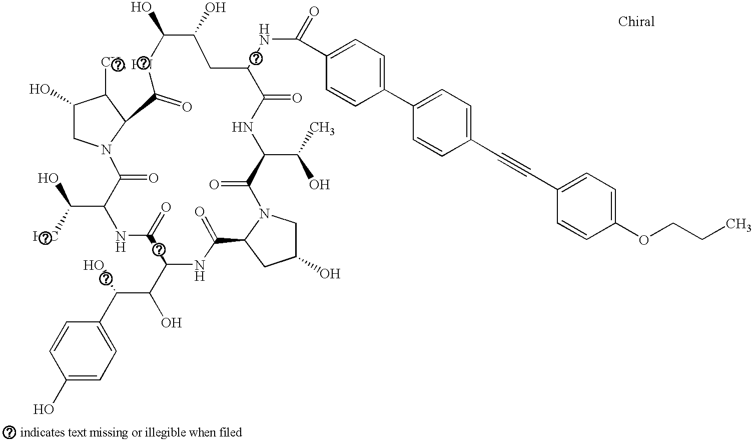 | Lilly | 1) Bartlett, MS. et al. | EP 561639 |
| | | | 33rd lntersci Conf | |
| | | | Antimicrob Agents | |
| | | | Chemother (Oct 17-20, | |
| | | | New Orleans) 1993, | |
| | | | Abst 369. | |
| 222 | 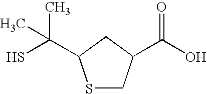 | Santen | | EP 558287 |
| 223 | 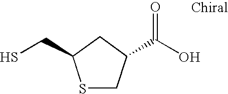 | Santen | | EP 558287 |
| 224 | 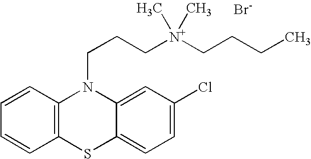 | ADIR | | EP 572308 |
| 225 | 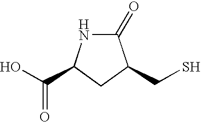 | Santen | | JP 93239045 |
| 226 | 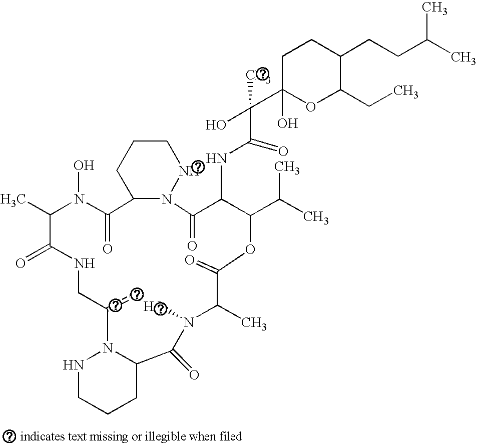 | Microbial Chemistry Research Foundation | Ueno, M. et al. J Antibiot 1993, 46(11): 1658. | |
| 227 | 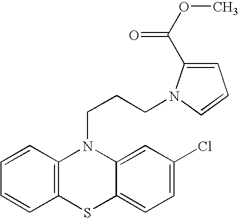 | ADIR | | EP 572308 |
| 228 | 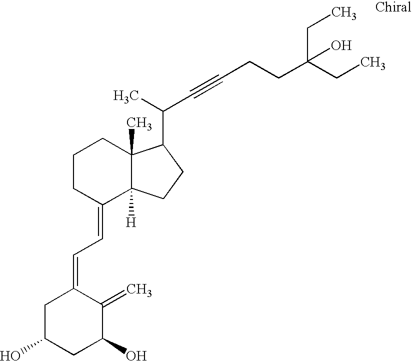 | Leo | | WO 9401398 |
| 229 | 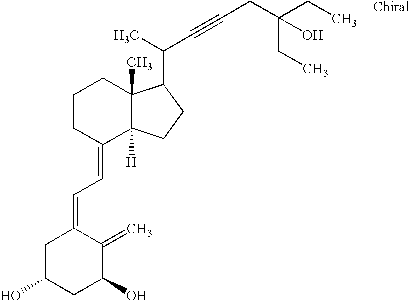 | Leo | | WO 9401398 |
| 230 | 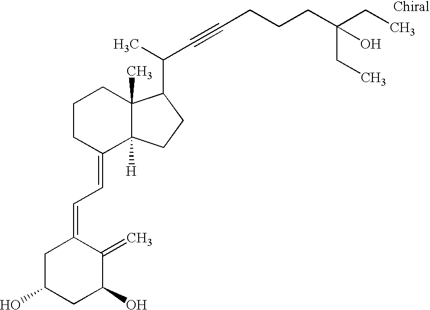 | Leo | | WO 9401398 |
| 231 | 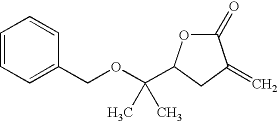 | Santen | | JP 93239045 |
| 232 | 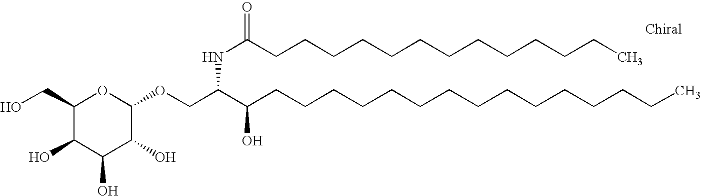 | Kirin Brewery | 1) Monta, M. et al. J Med Chem 1995, 38(12): 2176. | 1) EP 609437 |
| 233 | 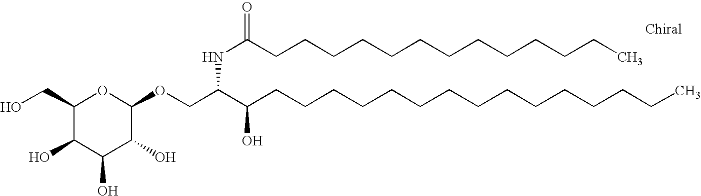 | Kirin Brewery | | 1) EP 609437 |
| 234 | 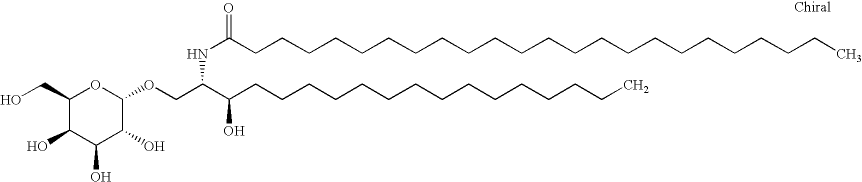 | Kirin Brewery | | 1) EP 609437 |
| 235 | 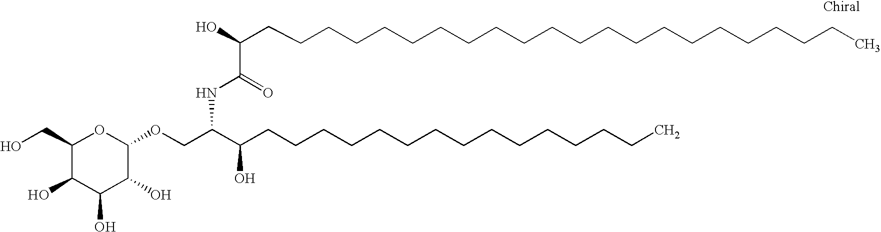 | Kirin Brewery | | EP 609437 |
| 236 | 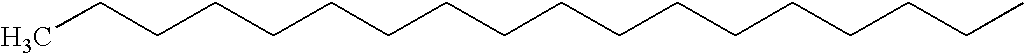 | Kirin Brewery | | EP 609437 |
| | 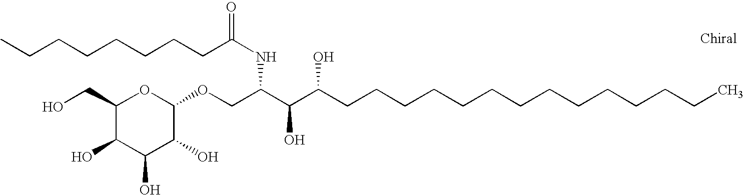 | | | |
| 237 | 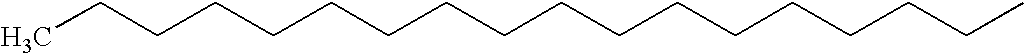 | Kirin Brewery | | EP 609437 |
| | 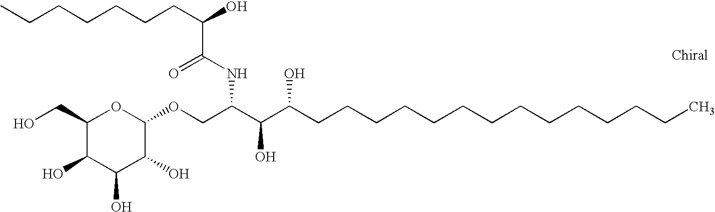 | | | |
| 238 | 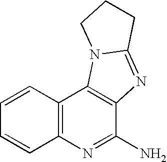 | 3M Pharmaceuticals | Lindstrom, K. J. et al. 211th ACS Natl Meet (March 24-28, New Orleans) 1996, Abst MEDI 210. | US 5482936 |
| 239 | 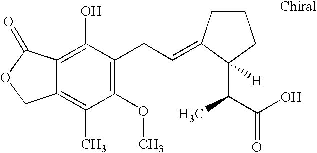 | Roche Bioscience | Smith, D. B. et al. J Org Chem 1996, 61(6): 2236. | WO 9522538 |
| 240 | 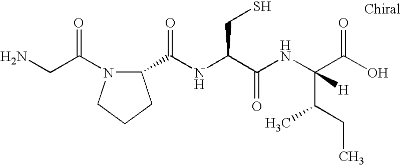 | AstraZeneca | | WO 9611943 |
| 241 | 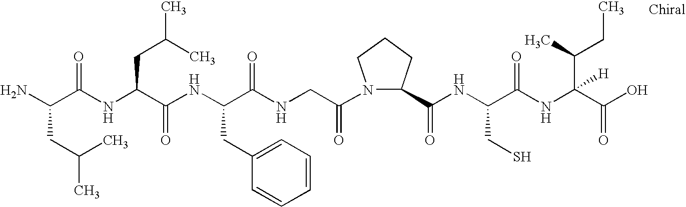 | AstraZeneca | | WO 9611943 |
| 242 | 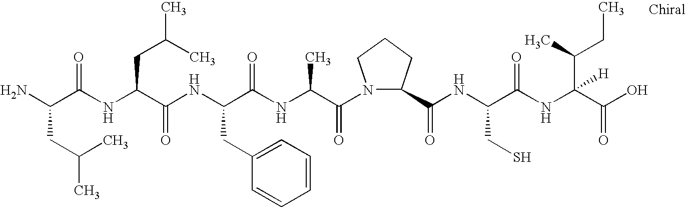 | AstraZeneca | | WO 9611943 |
| 243 | 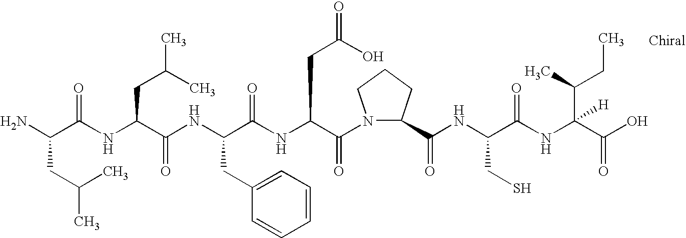 | AstraZeneca | | WO 9611943 |
| 244 | 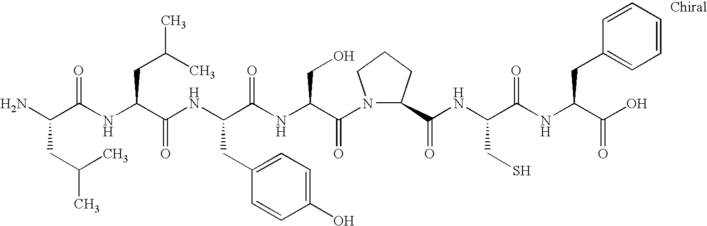 | AstraZeneca | | WO 9611943 |
| 245 | 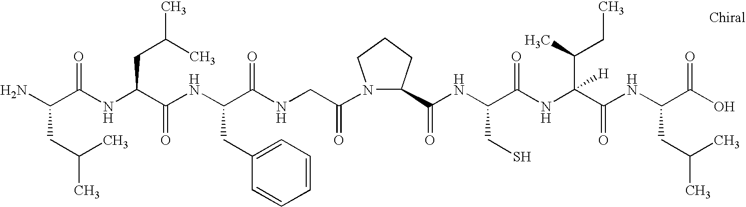 | AstraZeneca | | WO 9611943 |
| 246 | 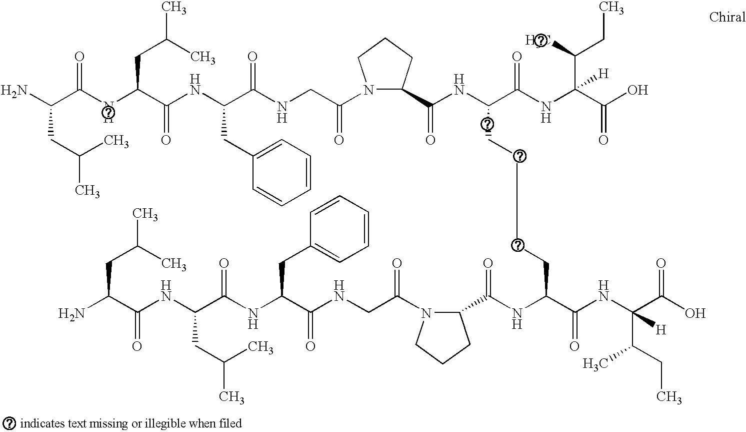 | AstraZeneca | | WO 9611943 |
| 247 | 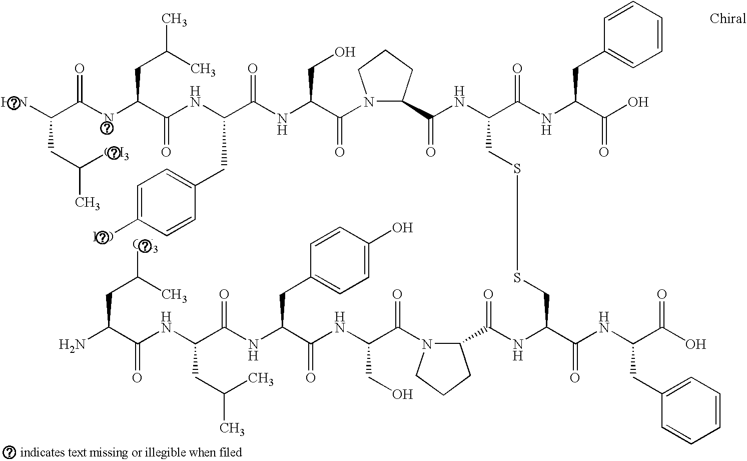 | AstraZeneca | | WO 9611943 |
| 248 | 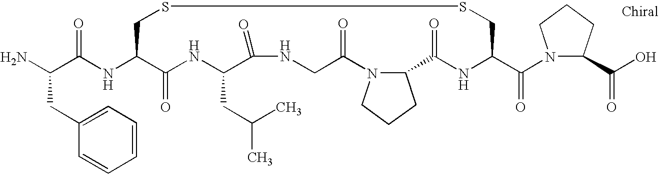 | AstraZeneca | | WO 9611943 |
| 249 | 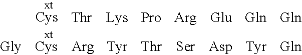 | Peptech | | WO 9612739 |
| 250 | 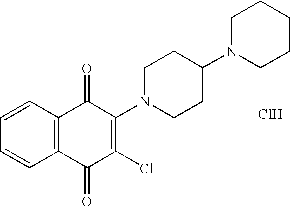 | Japan Tobacco | | JP 96113555 |
| 251 | 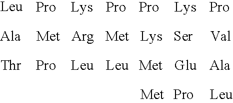 | Harvard College | | JP 96504177 |
| 252 | 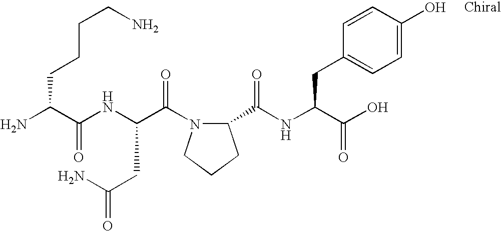 | Shire BioChem | | WO 9619494 |
| 253 | 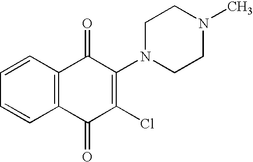 | Japan Tobacco | | JP 96113555 |
| 254 | 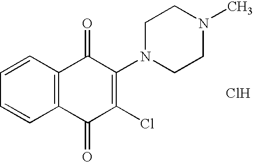 | Japan Tobacco | | JP 96113555 |
| 255 | 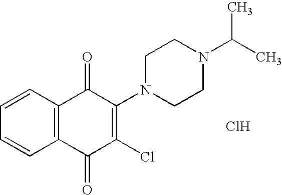 | Japan Tobacco | | JP 96113555 |
| 256 | 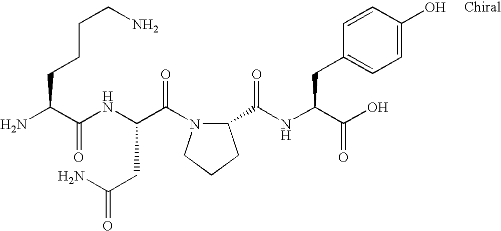 | Shire BioChem | | WO 9619494 |
| 257 | 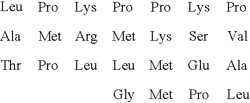 | Harvard College | | JP 96504177 |
| 258 | 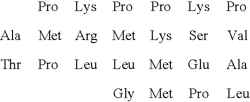 | Harvard College | | JP 96504177 |
| 259 | 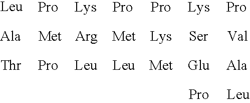 | Harvard College | | JP 96504177 |
| 260 | 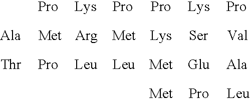 | Harvard College | | JP 96504177 |
| 261 | 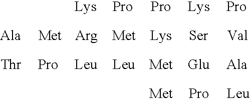 | Harvard College | | JP 96504177 |
| 262 | 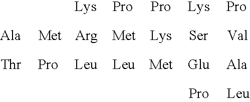 | Harvard College | | JP 96504177 |
| 263 | 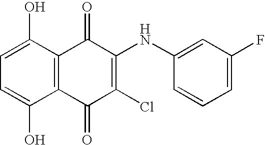 | Japan Tobacco | | JP 96113555 |
| 264 | 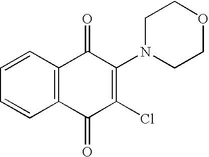 | Japan Tobacco | | JP 96113555 |
| 265 | 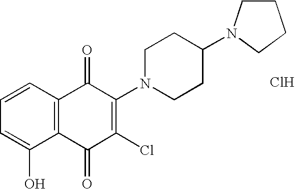 | Japan Tobacco | | JP 96113555 |
| 266 | 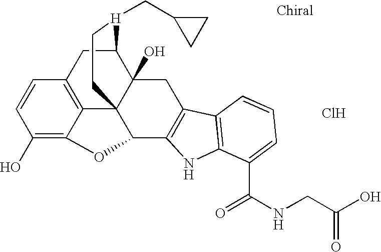 | Univ. Minnesota | | WO 9623793 |
| 267 | 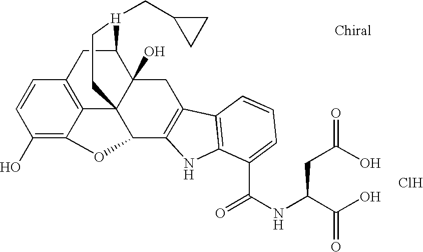 | Univ. Minnesota | | WO 9623793 |
| 268 | 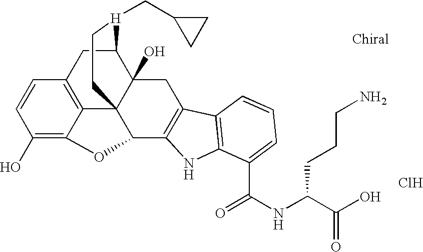 | Univ. Minnesota | | WO 9623793 |
| 269 | 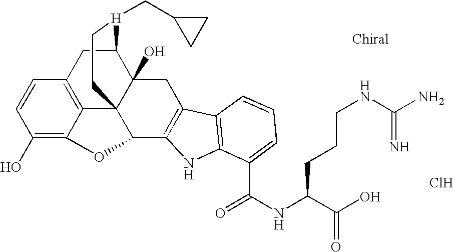 | Univ. Minnesota | | WO 9623793 |
| 270 | 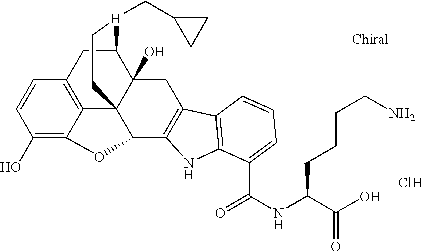 | Univ. Minnesota | | WO 9623793 |
| 271 | 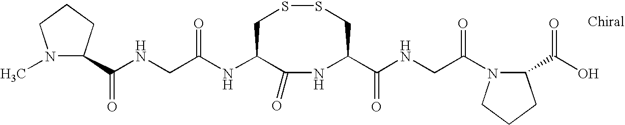 | AstraZeneca | | WO 9630397 |
| 272 | 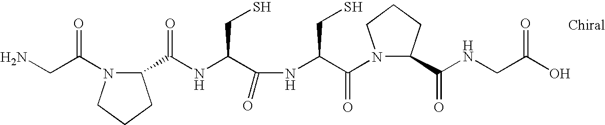 | AstraZeneca | | WO 9630397 |
| 273 | 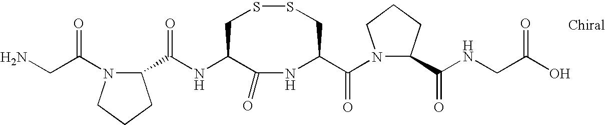 | AstraZeneca | | WO 9630397 |
| 274 | 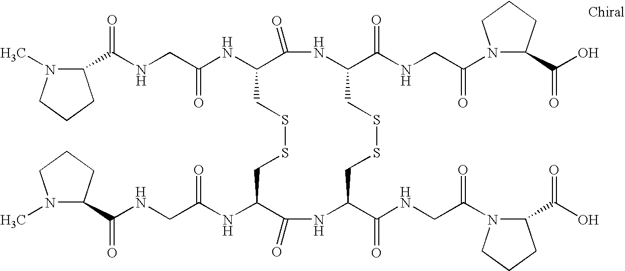 | AstraZeneca | | WO 9630397 |
| 275 | 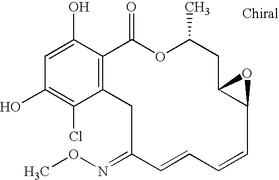 | Kyowa Hakko | | WO 9633989 |
| 276 | 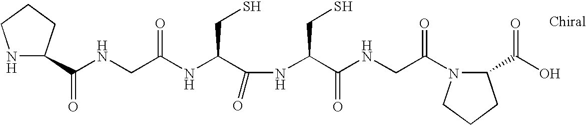 | AstraZeneca | | WO 9630397 |
| 277 | 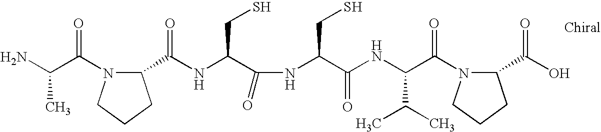 | AstraZeneca | | WO 9630397 |
| 278 | 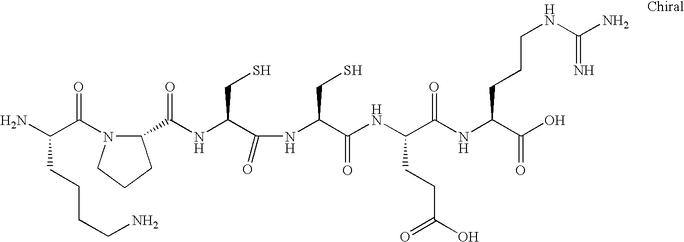 | AstraZeneca | | WO 9630397 |
| 279 | 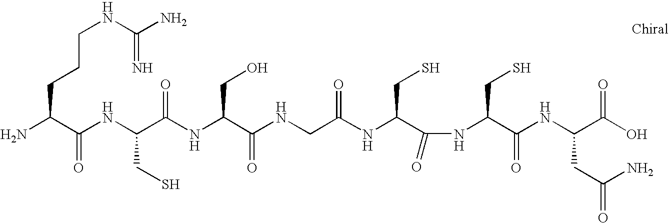 | AstraZeneca | | WO 9630397 |
| 280 | 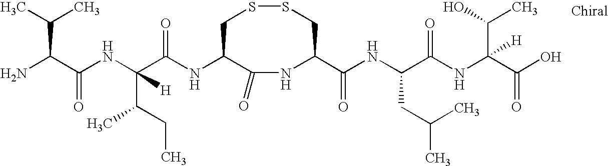 | AstraZeneca | | WO 9630397 |
| 281 | 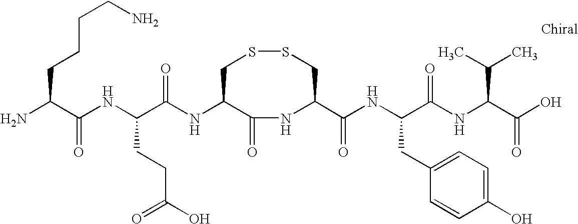 | AstraZeneca | | WO 9630397 |
| 282 | 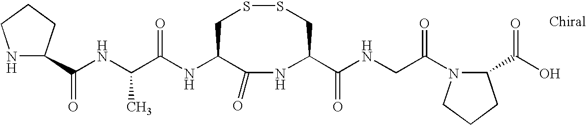 | AstraZeneca | | WO 9630397 |
| 283 | 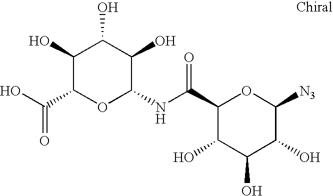 | Glycomed | | WO 9635700 |
| 284 | 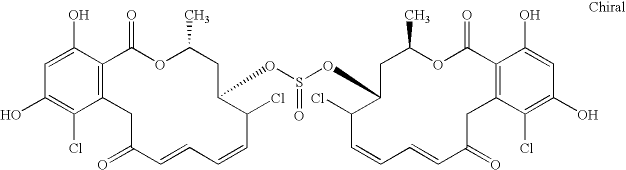 | Kyowa Hakko | | WO 9633989 |
| 285 | 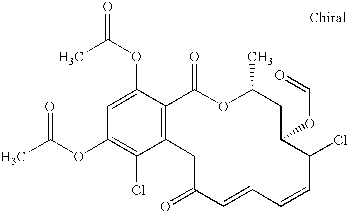 | Kyowa Hakko | | WO 9633989 |
| 286 | 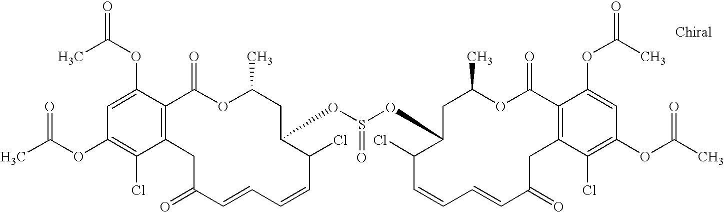 | Kyowa Hakko | | WO 9633989 |
| 287 | 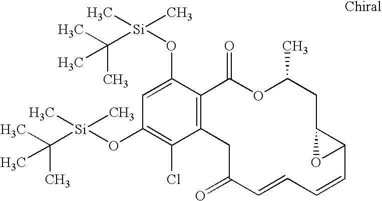 | Kyowa Hakko | | WO 9633989 |
| 288 | 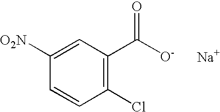 | Aston University | Kinchington, D. et al. 4th Conf Retroviruses Opportunistic Infect (Jan 22-26, Washington DC) 1997, Abst. | |
| 289 | 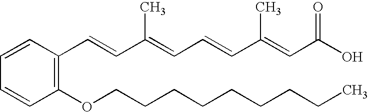 | Roche | 1) Fiedler-Nagy, C. et al. Agent Action 1989, 27(3-4): 313-5. | EP 169571 |
| 290 | 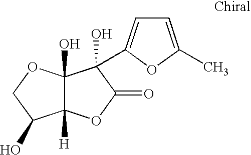 | NFCR | 1) Forest Laboratories, Inc. Annual Report 1994. | WO 9517890 |
| 291 | 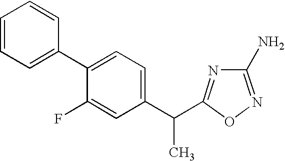 | Sumitomo Pharmaceuticals | | EP 248399 |
| 292 | 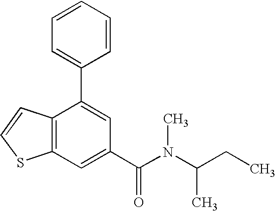 | Aventis Pharma | | EP 248734 |
| 293 | 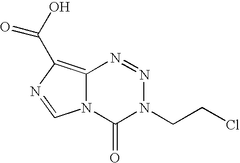 | May & Baker | | EP 252682 |
| 294 | 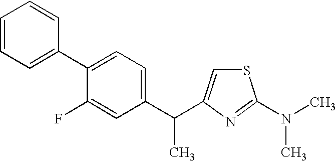 | Sumitomo Pharmaceuticals | | EP 248399 |
| 295 | 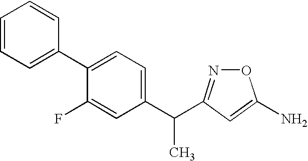 | Sumitomo Pharmaceuticals | | EP 248399 |
| 296 | 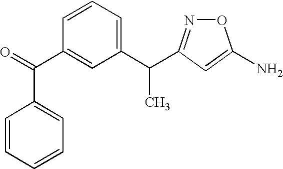 | Sumitomo Pharmaceuticals | | EP 248399 |
| 297 | 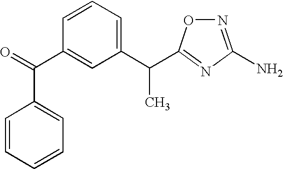 | Sumitomo Pharmaceuticals | | EP 248399 |
| 298 | 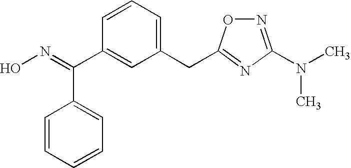 | Sumitomo Pharmaceuticals | | EP 248399 |
| 299 | 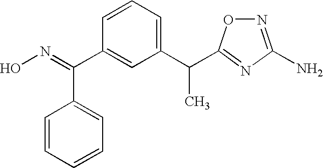 | Sumitomo Pharmaceuticals | | EP 248399 |
| 300 | 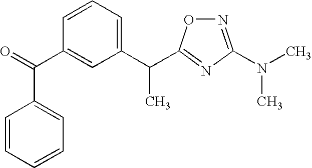 | Sumitomo Pharmaceuticals | | EP 248399 |
| 301 | 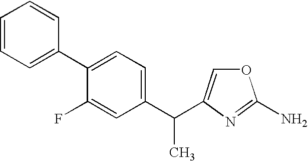 | Sumitomo Pharmaceuticals | | EP 248399 |
| 302 | 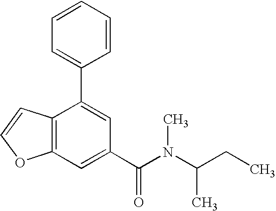 | Aventis Pharma | | EP 248734 |
| 303 | 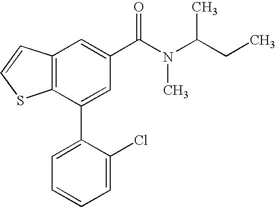 | Aventis Pharma | | EP 248734 |
| 304 | 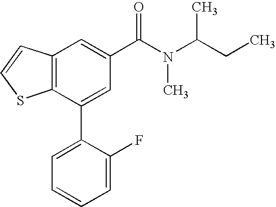 | Aventis Pharma | | EP 248734 |
| 305 | 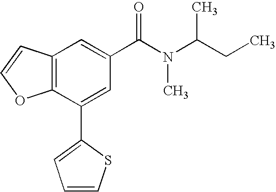 | Aventis Pharma | | EP 248734 |
| 306 | 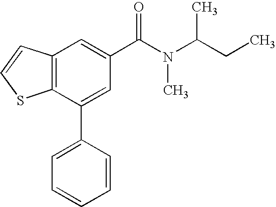 | Aventis Pharma | | EP 248734 |
| 307 | 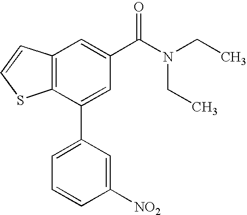 | Aventis Pharma | | EP 248734 |
| 308 | 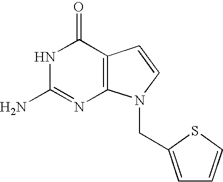 | Pfizer | | AU 8783281 |
| 309 | 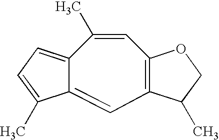 | Harbor Branch Found. | | US 4755529 |
| 310 | 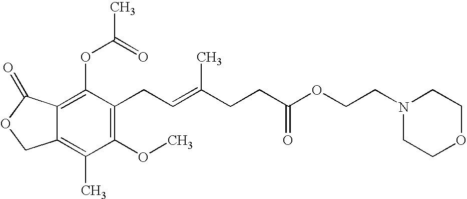 | Roche Bioscience | | AU 8782540 |
| 311 | 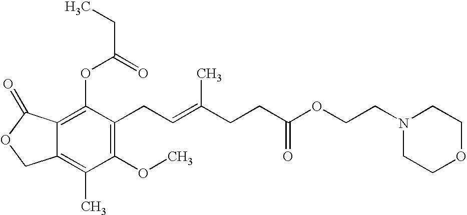 | Roche Bioscience | | AU 8782540 |
| 312 | 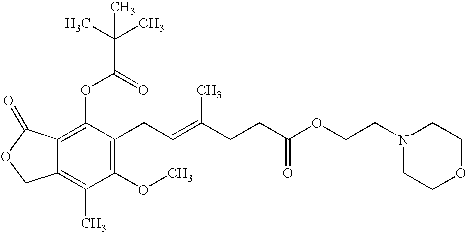 | Roche Bioscience | | AU 8782540 |
| 313 | 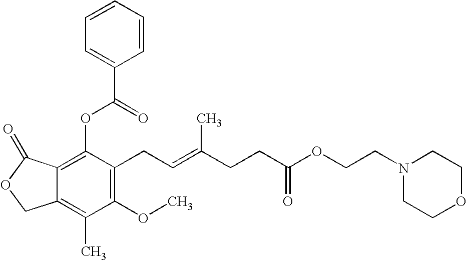 | Roche Bioscience | | AU 8782540 |
| 314 | 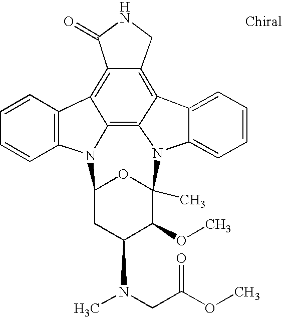 | Novartis | | EP 296110 |
| 315 | 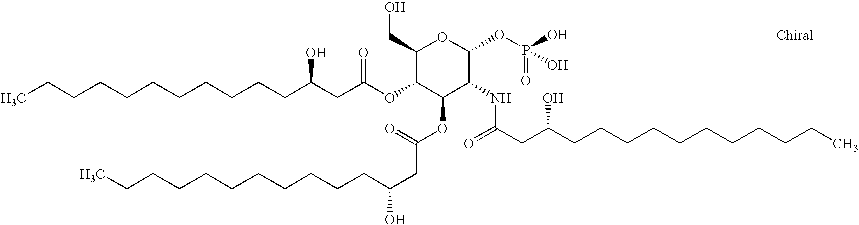 | Novartis | 1) Lam, C. et al. Antimicrob Agents Chemother 1991, 35(3): 500. | AU 8822785 |
| 316 | 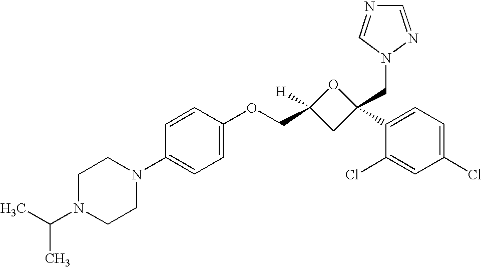 | Schering-Plough | | EP 318214 |
| 317 | 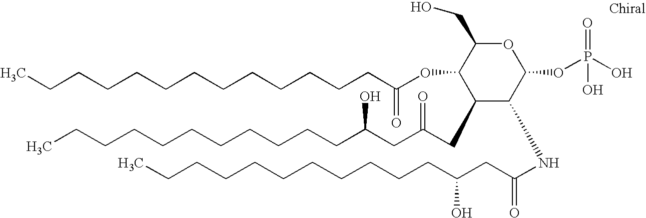 | Novartis | | AU 8822785 |
| 318 | 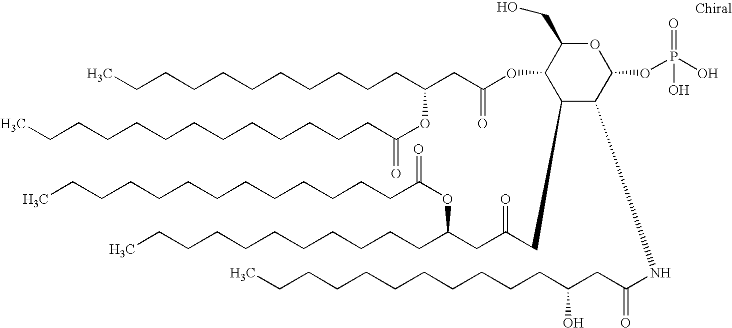 | Novartis | | AU 8822785 |
| 319 | 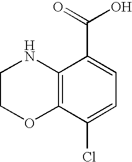 | Kyorin | | EP 310096 |
| 320 | 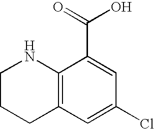 | Kyorin | | EP 310096 |
| 321 | 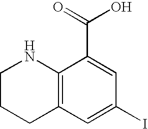 | Kyorin | | EP 310096 |
| 322 | 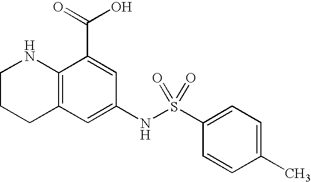 | Kyorin | | EP 310096 |
| 323 | 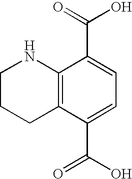 | Kyorin | | EP 310096 |
| 324 | 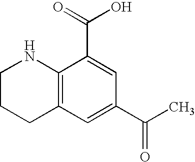 | Kyorin | | EP 310096 |
| 325 | 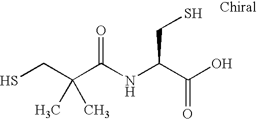 | Santen | | EP 326326 |
| 326 | 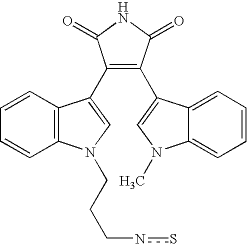 | Roche | | AU 8929658 |
| 327 | 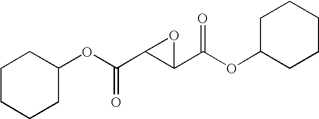 | Taisho | 1) Takeshita, K. et al. Int J Immunother 1988, 4(2): 97-106. | JP 79141750 |
| 328 | 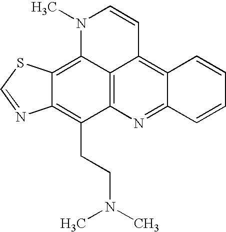 | Harbor Branch Found. | 1) Burres, N.S. et al. Proc Amer Assoc Cancer Res 1989, 30: Abst 1914. | EP 331320 |
| 329 | 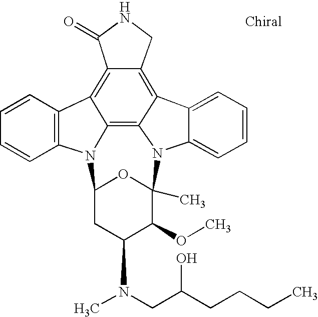 | Novartis | | EP 296110 |
| 330 | 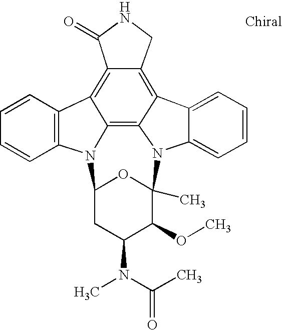 | Novartis | | EP 296110 |
| 331 | 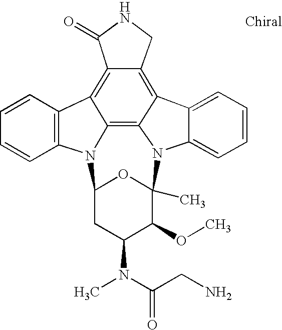 | Novartis | | EP 296110 |
| 332 | 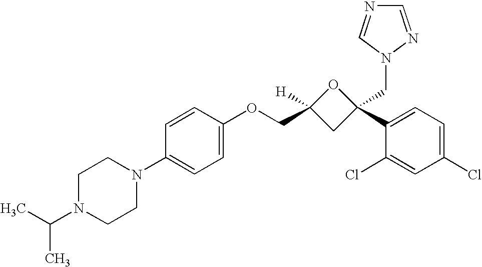 | Schering-Plough | | EP 318214 |
| 333 | 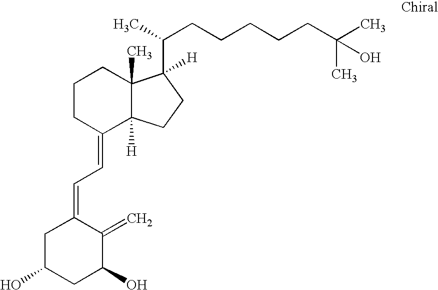 | Leo | | WO 8910351 |
| 334 | 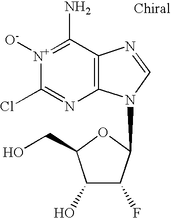 | Scripps Clinic Res. Found. | | WO 8908658 |
| 335 | 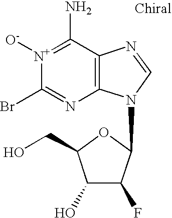 | Scripps Clinic Res. Found. | | WO 8908658 |
| 336 | 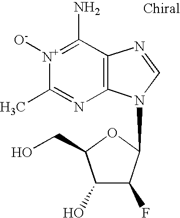 | Scripps Clinic Res. Found. | | WO 8908658 |
| 337 | 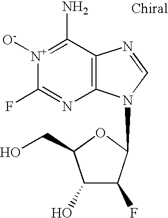 | Scripps Clinic Res. Found. | | WO 8908658 |
| 338 | 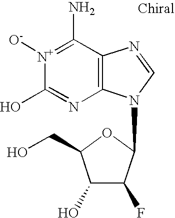 | Scripps Clinic Res. Found. | | WO 8908658 |
| 339 | 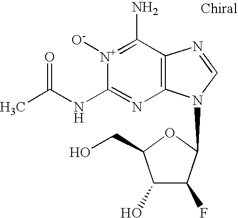 | Scripps Clinic Res. Found. | | WO 8908658 |
| 340 | 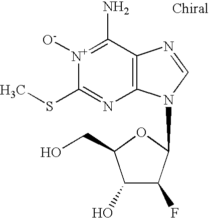 | Scripps Clinic Res. Found. | | WO 8908658 |
| 341 | 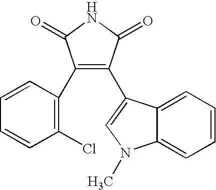 | Roche | | AU 8929658 |
| 342 | 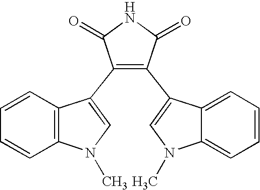 | Roche | | AU 8929658 |
| 343 | 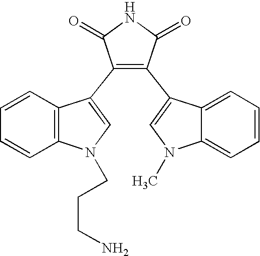 | Roche | | AU 8929658 |
| 344 | 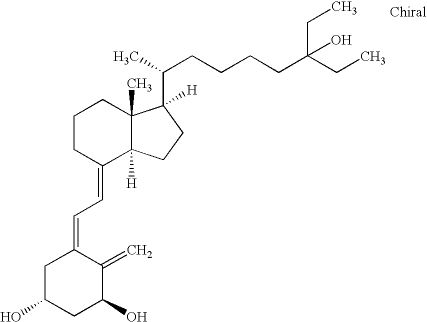 | Leo | | WO 8910351 |
| 345 | 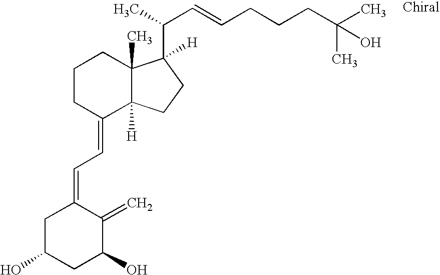 | Leo | | WO 8910351 |
| 346 | 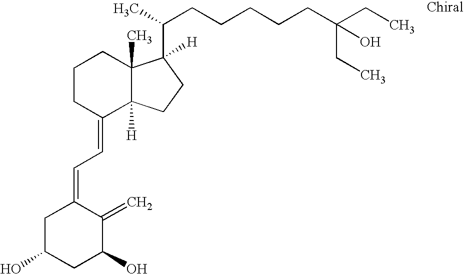 | Leo | | WO 8910351 |
| 347 | 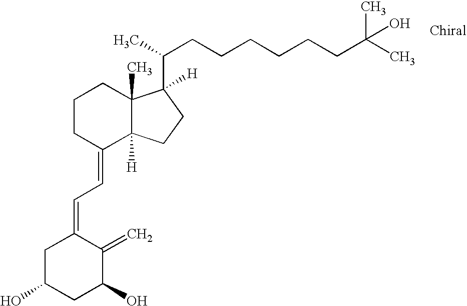 | Leo | | WO 8910351 |
| 348 | 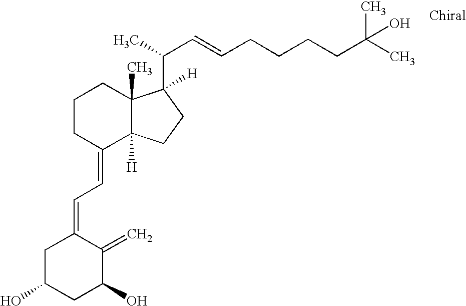 | Leo | | WO 8910351 |
| 349 | 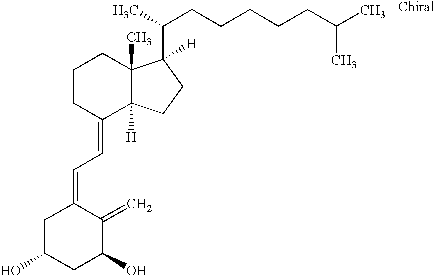 | Leo | | WO 8910351 |
| 350 | 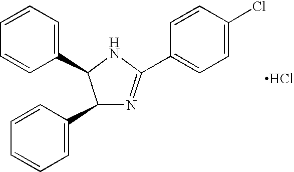 | Tanabe Seiyaku | 1) Ueno, M. et al. Jpn J Pharmacol 1992, 58(Suppl. 1): Abst O-210. | AU 8942368 |
| 351 | 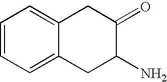 | Aventis Pharma | | EP 378456 |
| 352 | 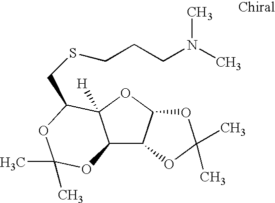 | Greenwich Pharm. | | AU 9047648 |
| 353 | 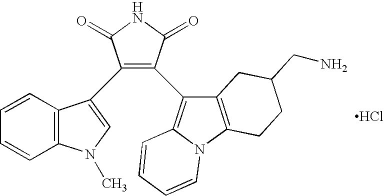 | Roche | | EP 384349 |
| 354 | 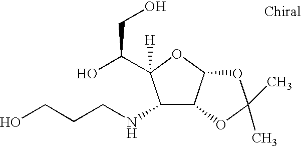 | Greenwich Pharm. | | AU 9047648 |
| 355 | 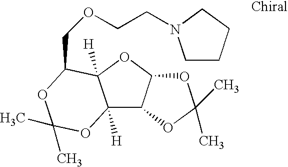 | Greenwich Pharm. | | AU 9047648 |
| 356 | 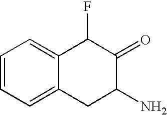 | Aventis Pharma | | EP 378456 |
| 357 | 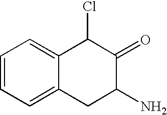 | Aventis Pharma | | EP 378456 |
| 358 | 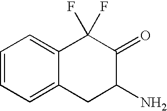 | Aventis Pharma | | EP 378456 |
| 359 | 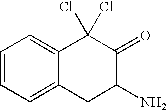 | Aventis Pharma | | EP 378456 |
| 360 | 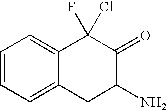 | Aventis Pharma | | EP 378456 |
| 361 | 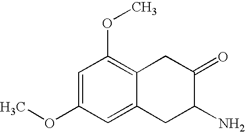 | Aventis Pharma | | EP 378456 |
| 362 | 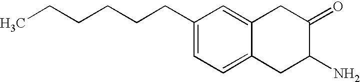 | Aventis Pharma | | EP 378456 |
| 363 | 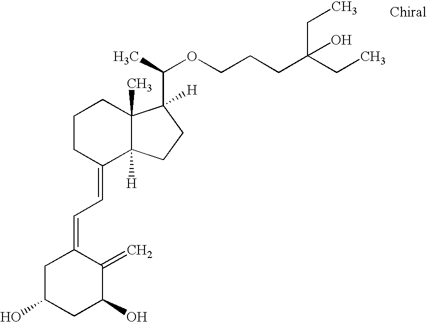 | Leo | Binderup, L. et al. Biochem Pharmacol 1991, 42(8): 1569. | EP 460032 |
| 364 | 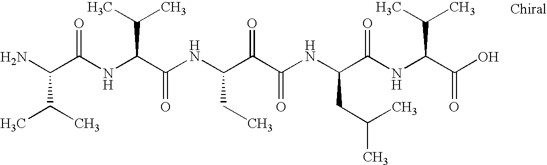 | Microbial Chemistry Research Foundation | Muraoka, Y. et al. 30th Intersci Conf Antimicrob Agents Chemother (Oct 21-24, Atlanta) 1990, Abst 801 | |
| 365 | 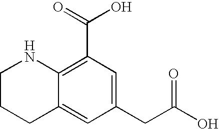 | Kyorin | | AU 9057029 |
| 366 | 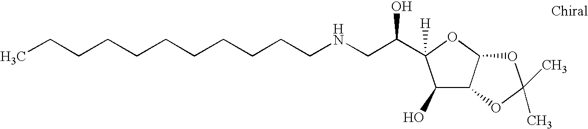 | Greenwich Pharm. | | AU 9057691 |
| 367 | 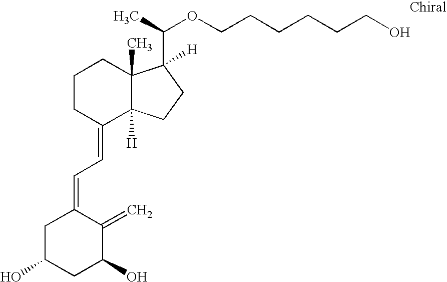 | Leo | | EP 460032 |
| 368 | 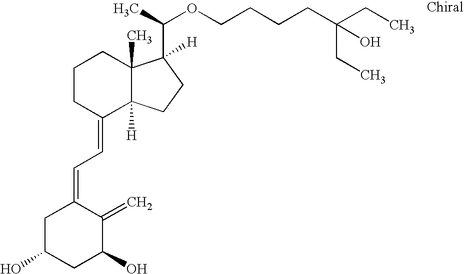 | Leo | | EP 460032 |
| 369 | 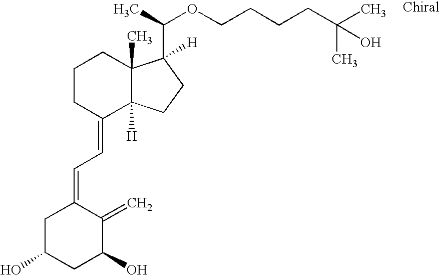 | Leo | | EP 460032 |
| 370 | 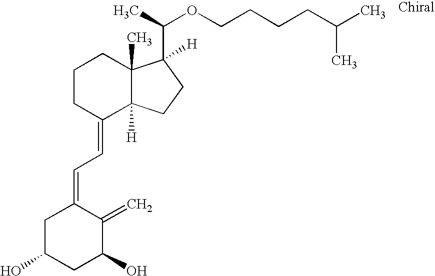 | Leo | | EP 460032 |
| 371 | 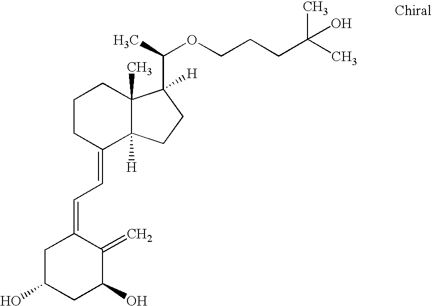 | Leo | | EP 460032 |
| 372 | 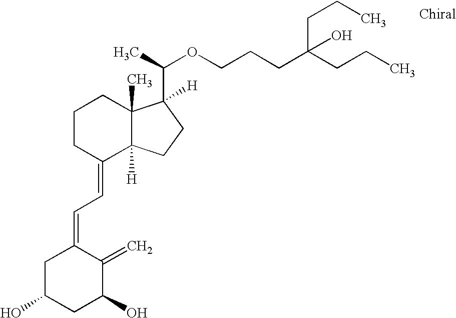 | Leo | | EP 460032 |
| 373 | 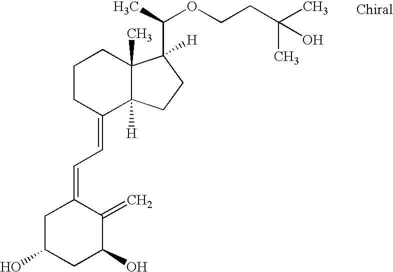 | Leo | | EP 460032 |
| 374 | 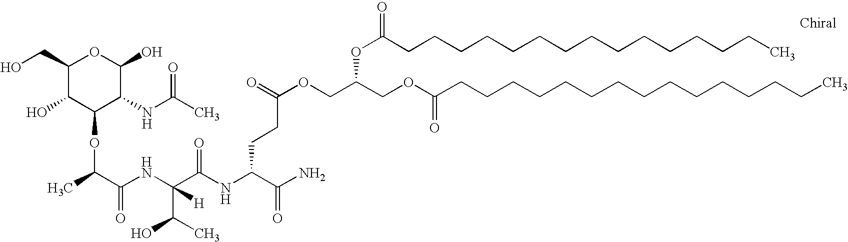 | Novartis | Kricek. F. et al. Immunopharmacology 1997, 36(1): 27. | AU 9057875 |
| 375 | 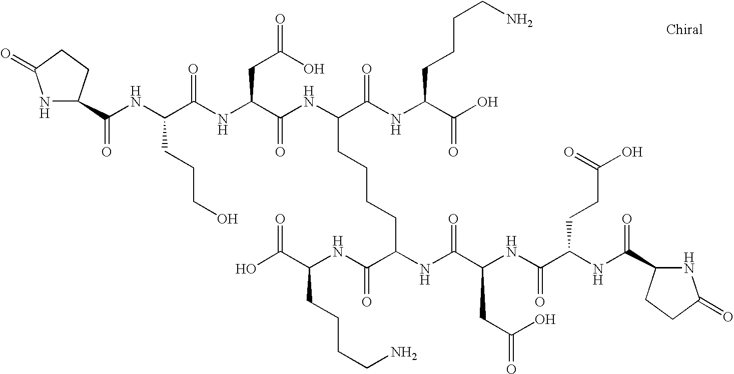 | Amersham Health | 1) Frey, C. L. et al. 31st Intersci Conf Antimicrob Agents Chemother (Sept 29-Oct 2, Chicago) 1991, Abst 85. | AU 9059014 |
| 376 | 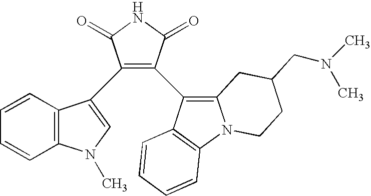 | Roche | | EP 384349 |
| 377 | 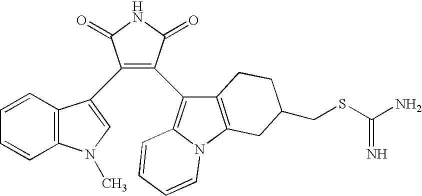 | Roche | | EP 384349 |
| 378 | 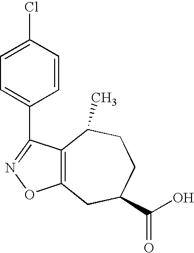 | Roche | | AU 9059154 |
| 379 | 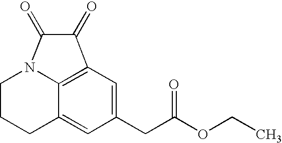 | Kyorin | | AU 9057029 |
| 380 | 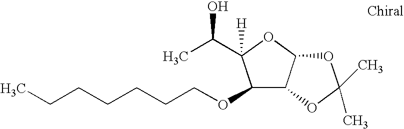 | Greenwich Pharm. | | AU 9057691 |
| 381 | 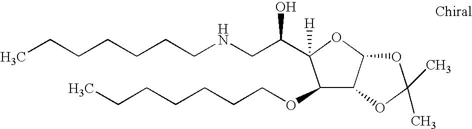 | Greenwich Pharm. | | AU 9057691 |
| 382 | 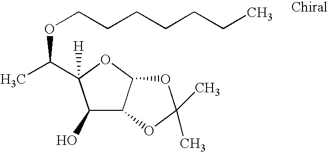 | Greenwich Pharm. | | AU 9057691 |
| 383 | 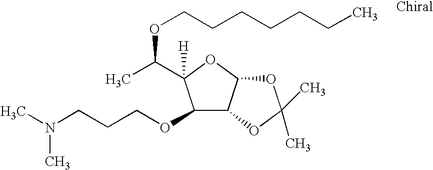 | Greenwich Pharm. | | AU 9057691 |
| 384 | 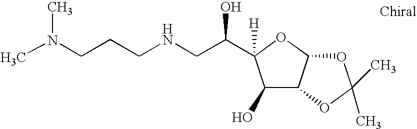 | Greenwich Pharm. | | AU 9057691 |
| 385 | 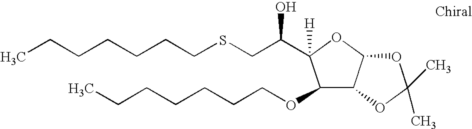 | Greenwich Pharm. | | AU 9057691 |
| 386 | 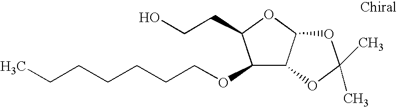 | Greenwich Pharm. | | AU 9057691 |
| 387 | 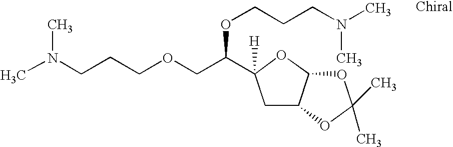 | Greenwich Pharm. | | AU 9057691 |
| 388 | 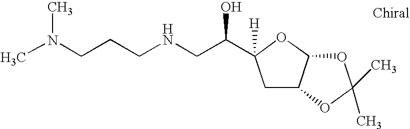 | Greenwich Pharm. | | AU 9057691 |
| 389 | 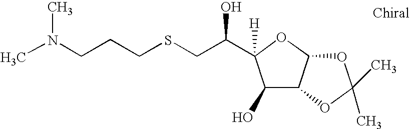 | Greenwich Pharm. | | AU 9057691 |
| 390 | 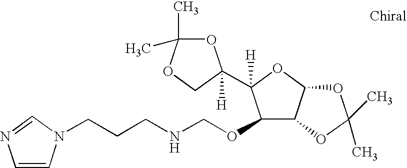 | Greenwich Pharm. | | AU 9057691 |
| 391 | 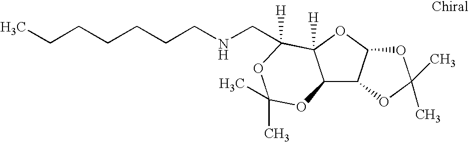 | Greenwich Pharm. | | AU 9057691 |
| 392 | 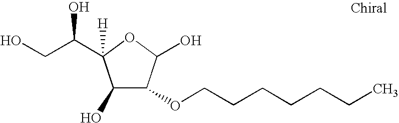 | Greenwich Pharm. | | AU 9057691 |
| 393 | 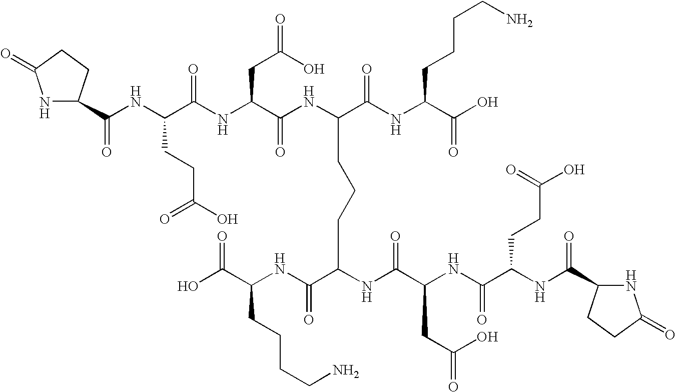 | Amersham Health | | AU 9059014 |
| 394 | 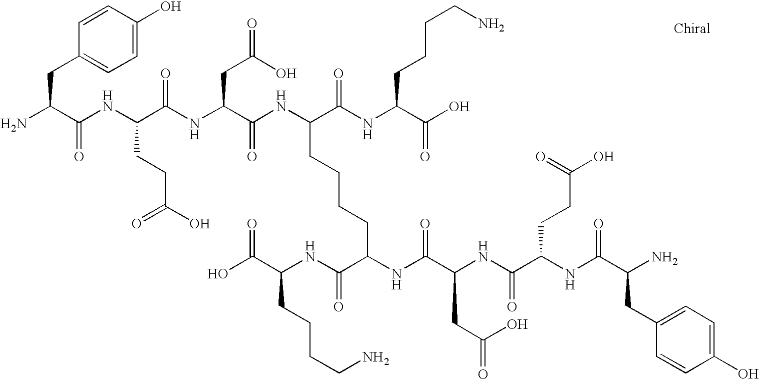 | Amersham Health | | AU 9059014 |
| 395 | 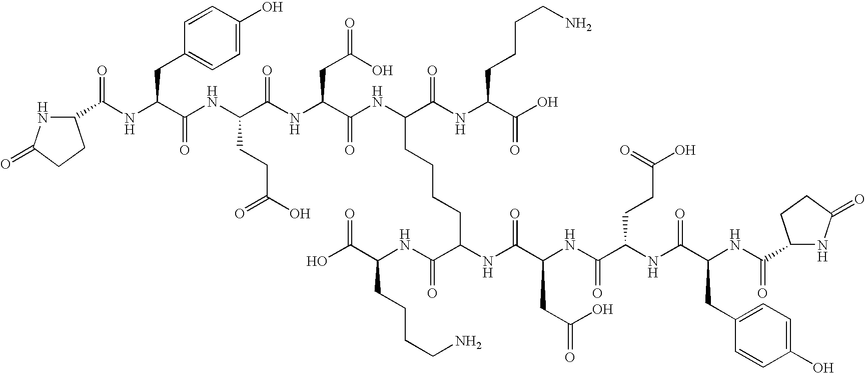 | Amersham Health | | AU 9059014 |
| 396 | 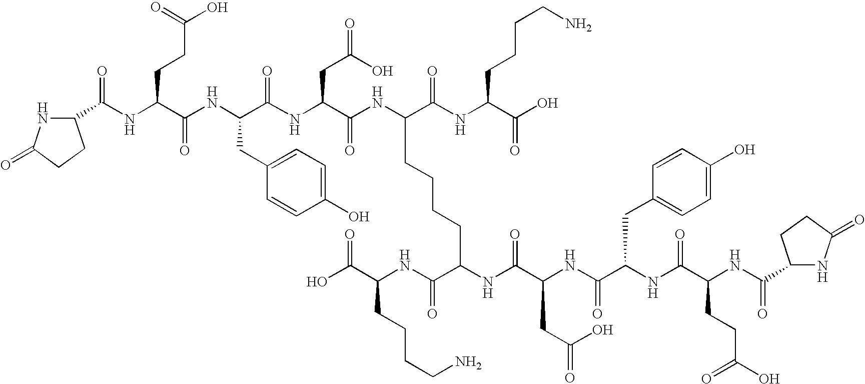 | Amersham Health | | AU 9059014 |
| 397 | 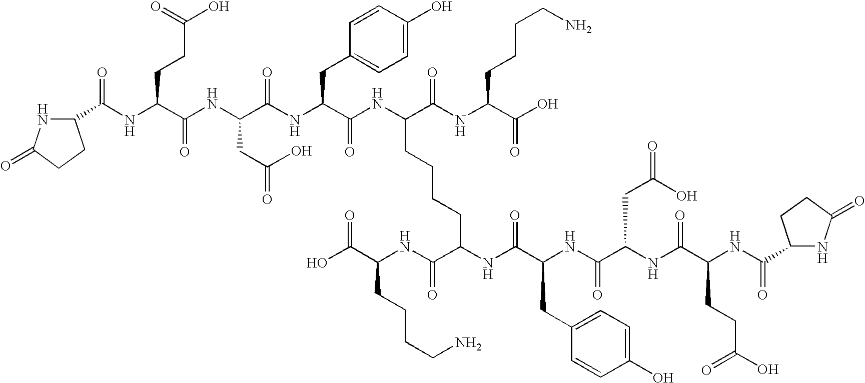 | Amersham Health | | AU 9059014 |
| 398 | 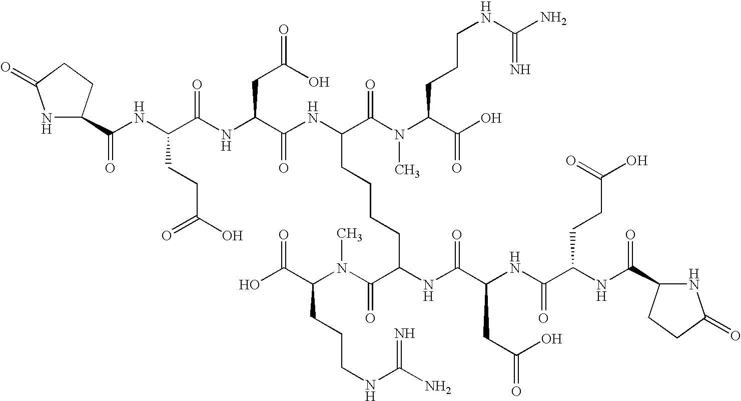 | Amersham Health | | AU 9059014 |
| 399 | 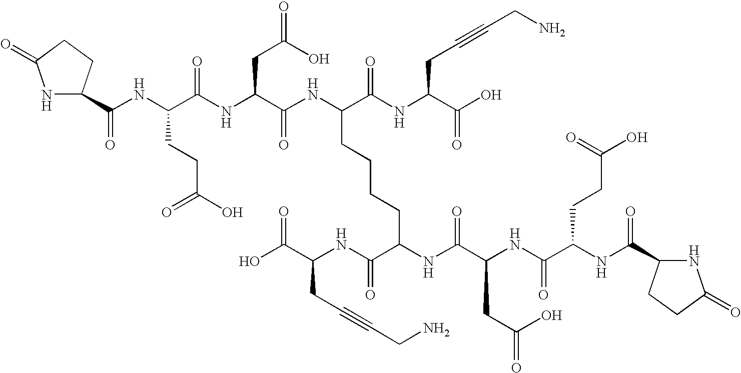 | Amersham Health | | AU 9059014 |
| 400 | 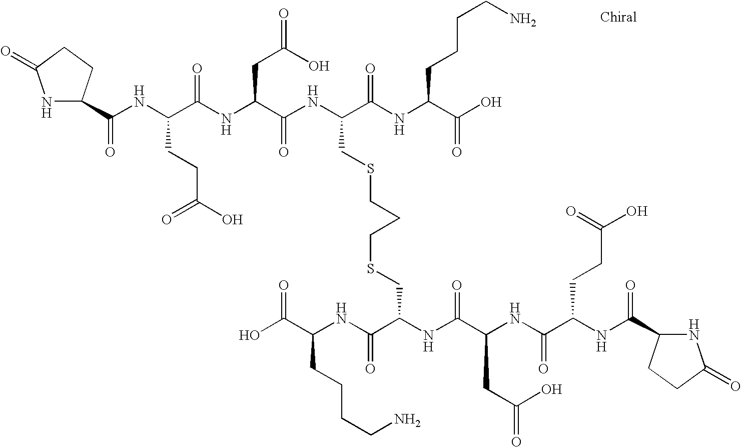 | Amersham Health | | AU 9059014 |
| 401 | 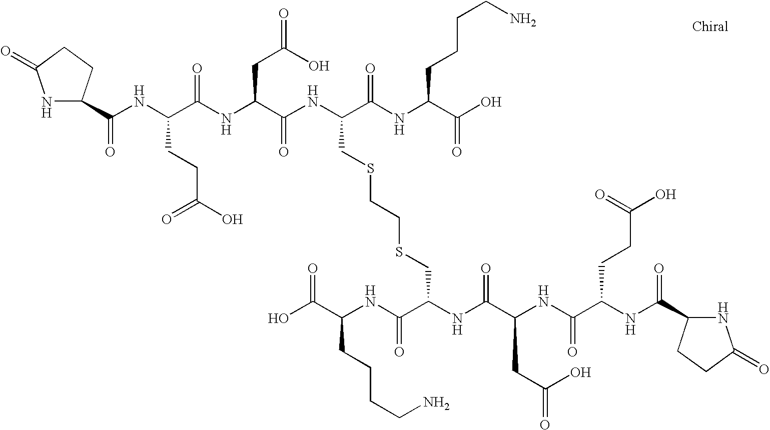 | Amersham Health | | AU 9059014 |
| 402 | 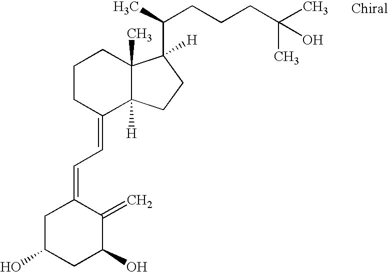 | Leo | 1) Elstner, E. et al. Blood 1994, 84(6): 1960. | EP 479871 |
| 403 | 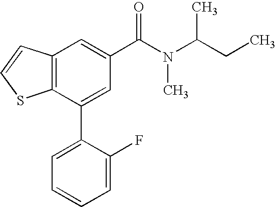 | Leo | | WO 9100855 |
| 404 | 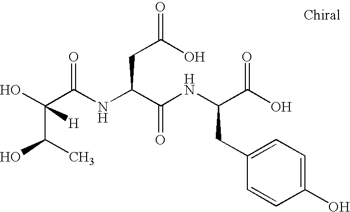 | SPA | | EP 421074 |
| 405 | 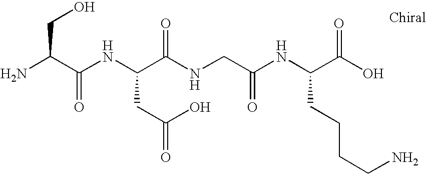 | Hitachi Chemical | | EP 421682 |
| 406 | 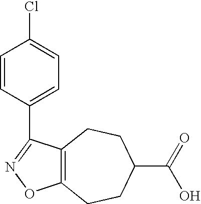 | Roche | | AU 9059154 |
| 407 | 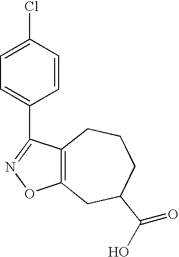 | Roche | | AU 9059154 |
| 408 | 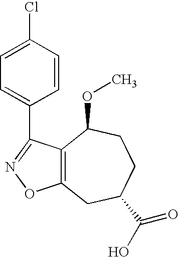 | Roche | | AU 9059154 |
| 409 | 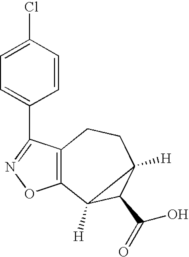 | Roche | | AU 9059154 |
| 410 | 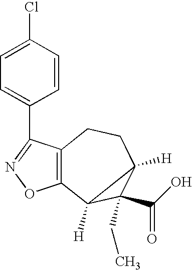 | Roche | | AU 9059154 |
| 411 | 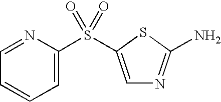 | Fujisawa | 1) Manda, T. et al. Jpn J Pharmacol 1997, 73(Suppl. 1): Abst P-140. | EP 412404 |
| 412 | 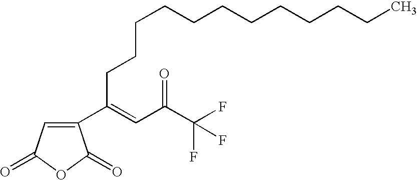 | Allergan | | US 5013850 |
| 413 | 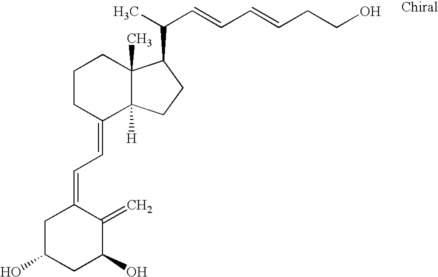 | Leo | | WO 9100855 |
| 414 | 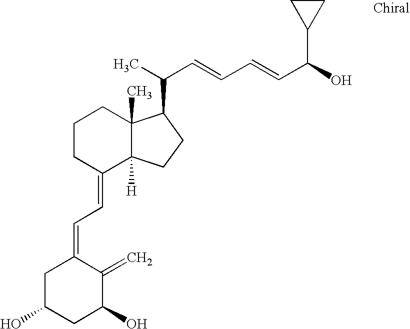 | Leo | | WO 9100855 |
| 415 | 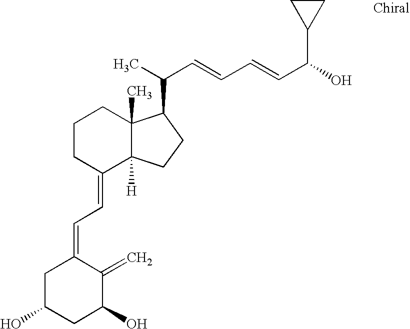 | Leo | | WO 9100855 |
| 416 | 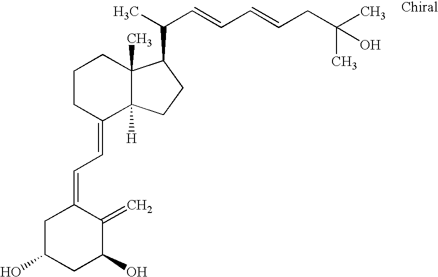 | Leo | | WO 9100855 |
| 417 | 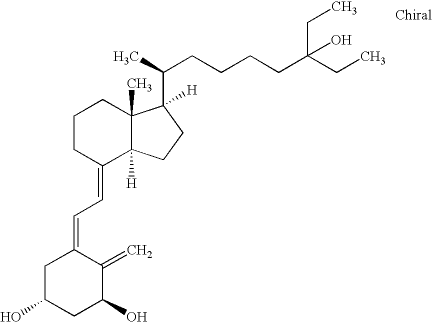 | Leo | | EP 479871 |
| 418 | 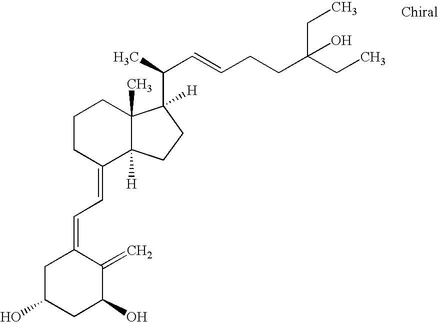 | Leo | | EP 479871 |
| 419 | 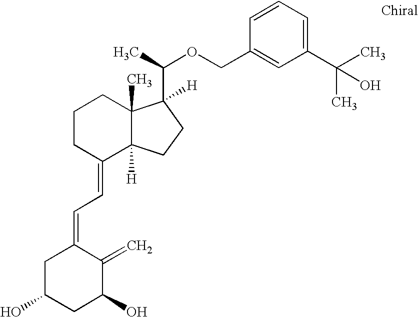 | Leo | | WO 9109841 |
| 420 | 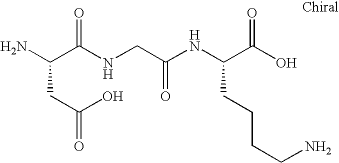 | Hitachi Chemical | | EP 421682 |
| 421 | 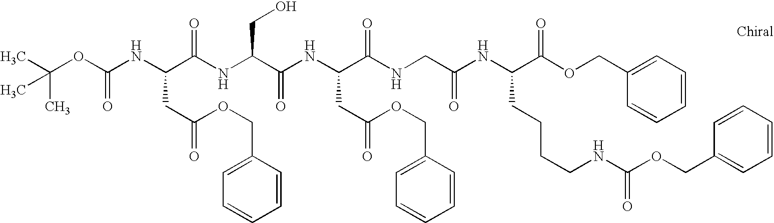 | Hitachi Chemical | | EP 421682 |
| 422 | 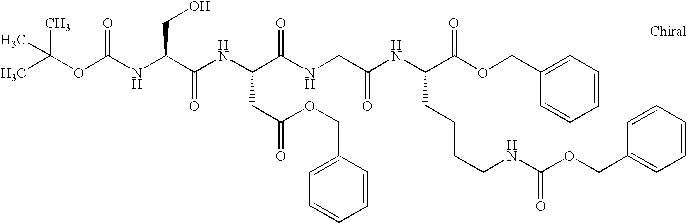 | Hitachi Chemical | | EP 421682 |
| 423 | 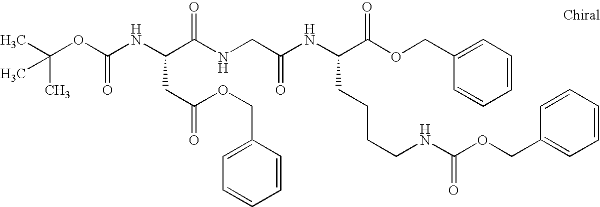 | Hitachi Chemical | | EP 421682 |
| 424 | 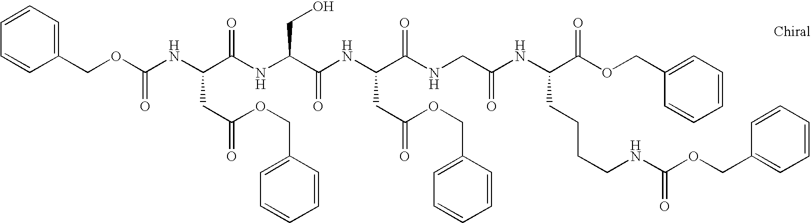 | Hitachi Chemical | | EP 421682 |
| 425 | 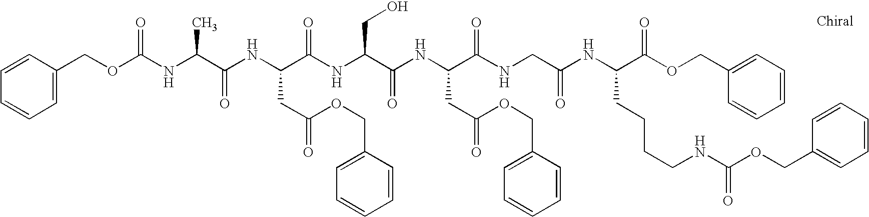 | Hitachi Chemical | | EP 421682 |
| 426 | 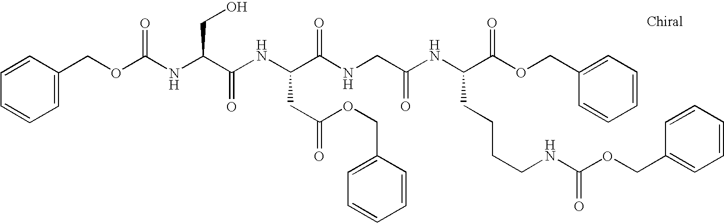 | Hitachi Chemical | | EP 421682 |
| 427 | 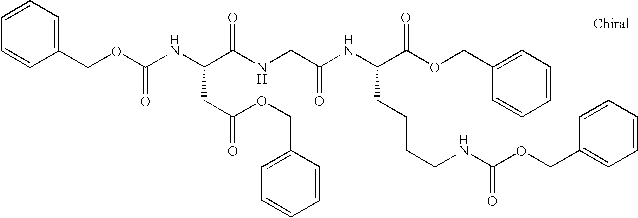 | Hitachi Chemical | | EP 421682 |
| 428 | 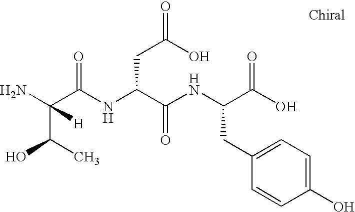 | SPA | | EP 421074 |
| 429 | 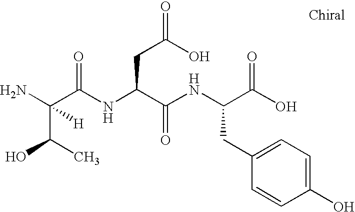 | SPA | | EP 421074 |
| 430 | 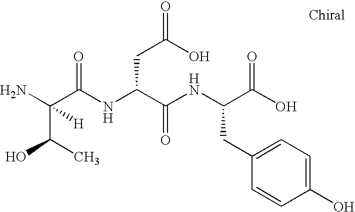 | SPA | | EP 421074 |
| 431 | 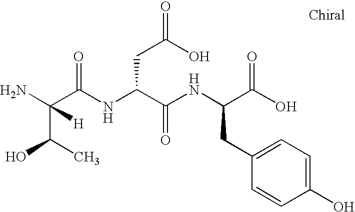 | SPA | | EP 421074 |
| 432 | 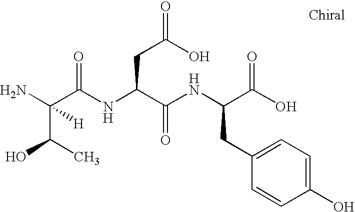 | SPA | | EP 421074 |
| 433 | 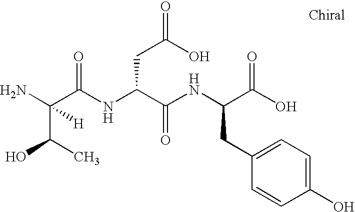 | SPA | | EP 421074 |
| 434 | 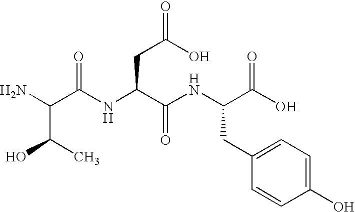 | SPA | | EP 421074 |
| 435 | 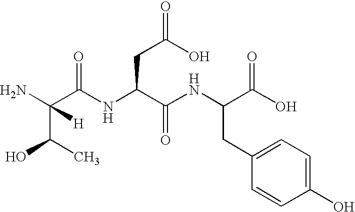 | SPA | | EP 421074 |
| 436 | 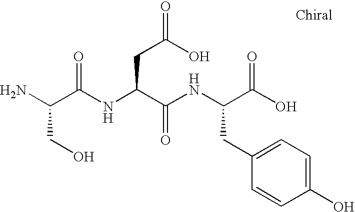 | SPA | | EP 421074 |
| 437 | 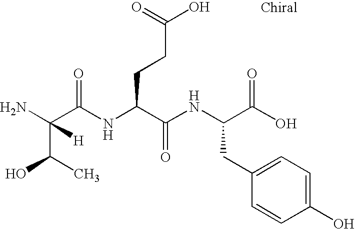 | SPA | | EP 421074 |
| 438 | 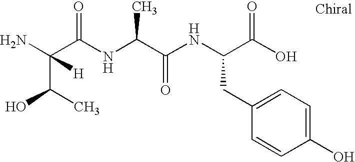 | SPA | | EP 421074 |
| 439 | 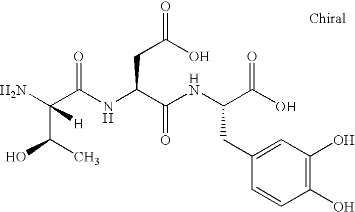 | SPA | | EP 421074 |
| 440 | 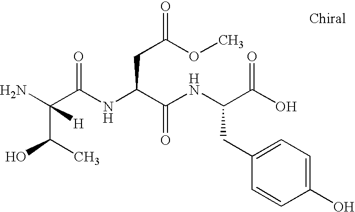 | SPA | | EP 421074 |
| 441 | 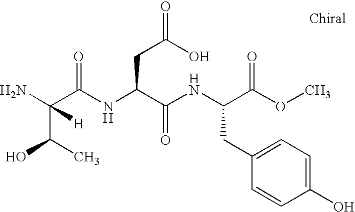 | SPA | | EP 421074 |
| 442 | 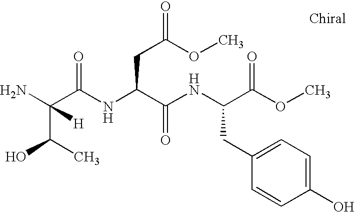 | SPA | | EP 421074 |
| 443 | 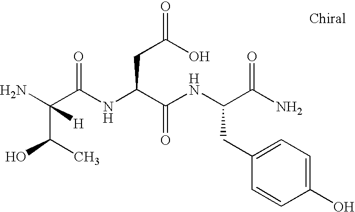 | SPA | | EP 421074 |
| 444 | 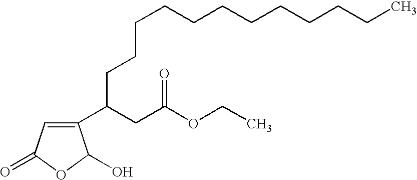 | Allergan | | US 5013850 |
| 445 | 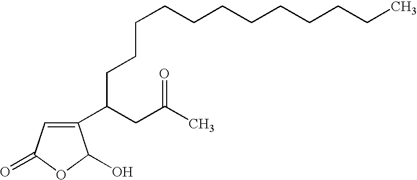 | Allergan | | US 5013850 |
| 446 | 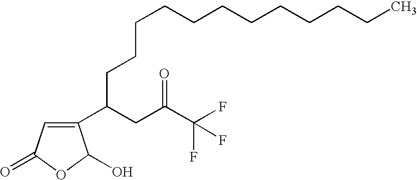 | Allergan | | US 5013850 |
| 447 | 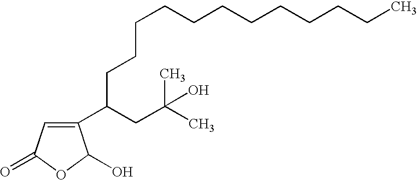 | Allergan | | US 5013850 |
| 448 | 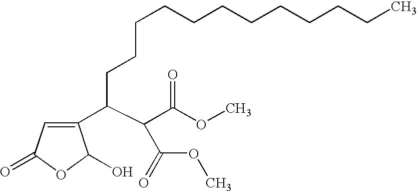 | Allergan | | US 5013850 |
| 449 | 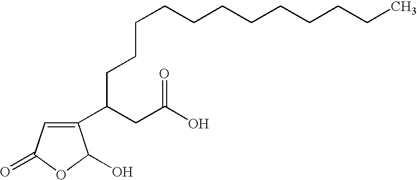 | Allergan | | US 5013850 |
| 450 | 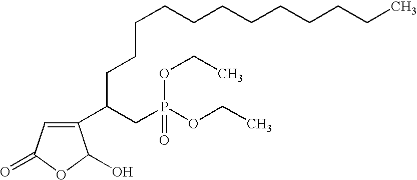 | Allergan | | US 5013850 |
| 451 | 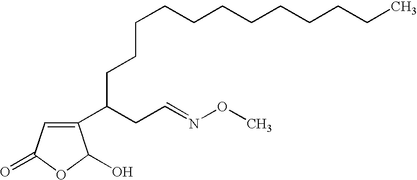 | Allergan | | US 5013850 |
| 452 | 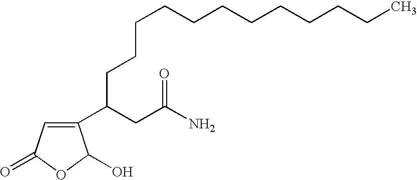 | Allergan | | US 5013850 |
| 453 | 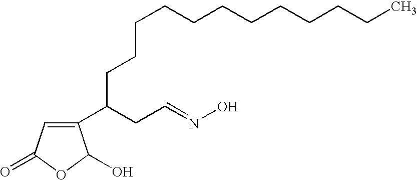 | Allergan | | US 5013850 |
| 454 | 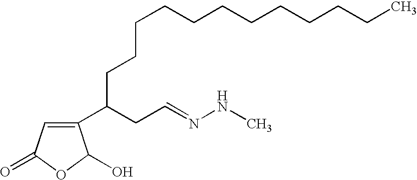 | Allergan | | US 5013850 |
| 455 | 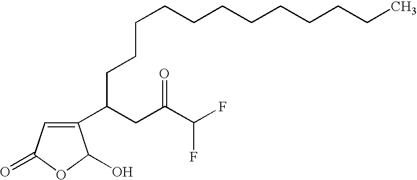 | Allergan | | US 5013850 |
| 456 | 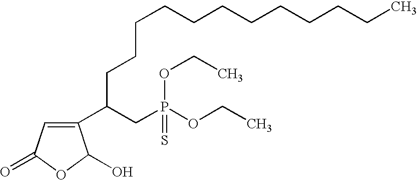 | Allergan | | US 5013850 |
| 457 | 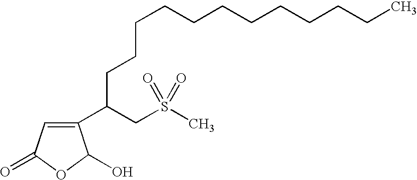 | Allergan | | US 5013850 |
| 458 | 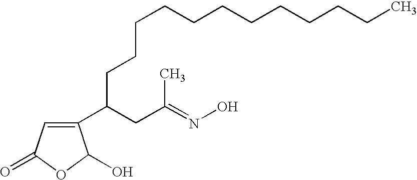 | Allergan | | US 5013850 |
| 459 | 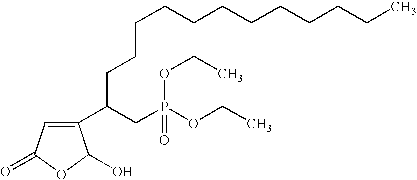 | Allergan | | US 5013850 |
| 460 | 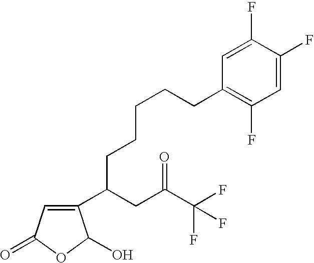 | Allergan | | US 5013850 |
| 461 | 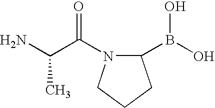 | New England Med. Center Hosp. | | WO 9116339 |
| 462 | 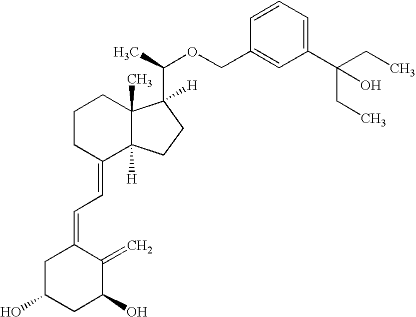 | Leo | | EP 506794 |
| 463 | 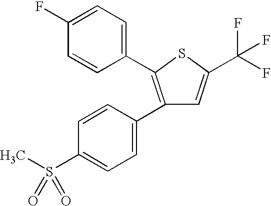 | Fujisawa | | WO 9119708 |
| 464 | 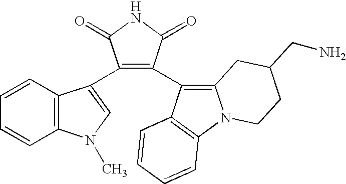 | Roche | 1) Hill, C. H. 6th SCI- RSC Med Chem Symp (Sept 8-11, Cambridge) 1991, Abst S18. | EP 384349 |
| 465 | 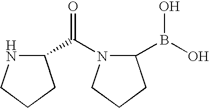 | New England Med. Center Hosp. | | WO 9116339 |
| 466 | 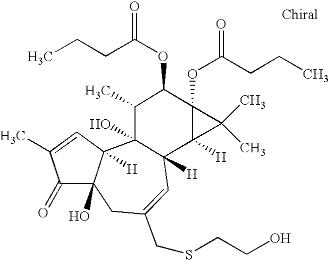 | Alder | | US 5145842 |
| 467 | 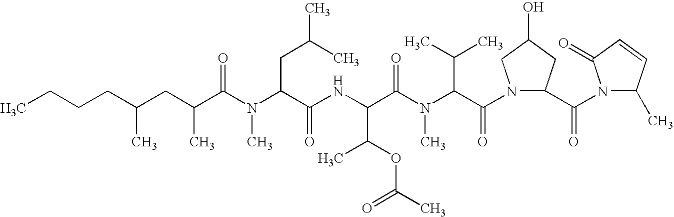 | Harbor Branch Found. | | US 5091368 |
| 468 | 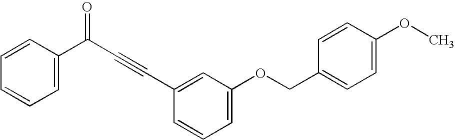 | Aventis Pharma | | EP 476658 |
| 469 | 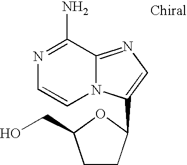 | Merck & Co. | | EP 480713 |
| 470 | 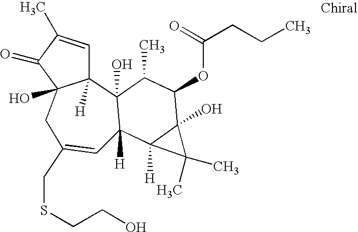 | Alder | | US 5145842 |
| 471 | 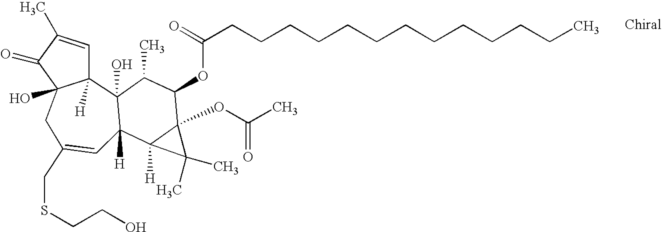 | Alder | | US 5145842 |
| 472 | 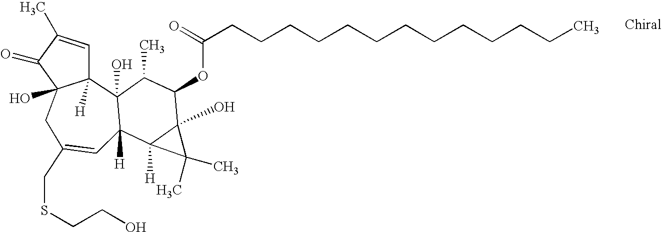 | Alder | | US 5145842 |
| 473 | 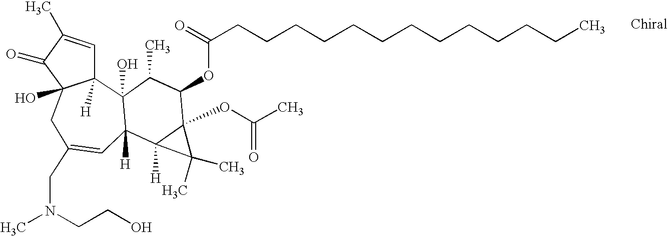 | Alder | | US 5145842 |
| 474 | 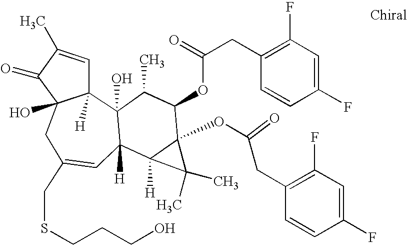 | Alder | US 5145842 | |
| 475 | 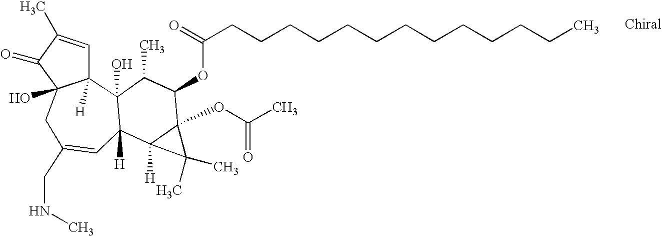 | Alder | | US 5145842 |
| 476 | 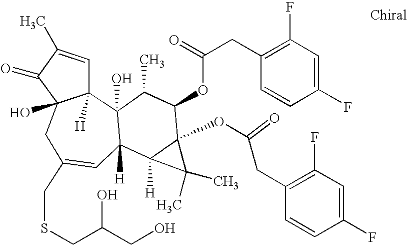 | Alder | | US 5145842 |
| 477 | 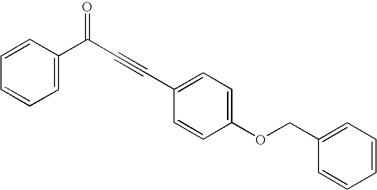 | Aventis Pharma | | EP 476658 |
| 478 | 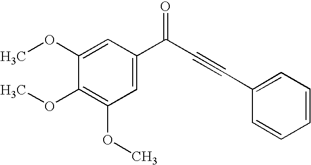 | Aventis Pharma | | EP 476658 |
| 479 | 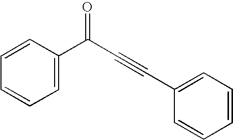 | Aventis Pharma | | EP 476658 |
| 480 | 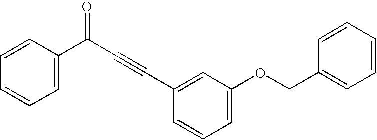 | Aventis Pharma | | EP 476658 |
| 481 | 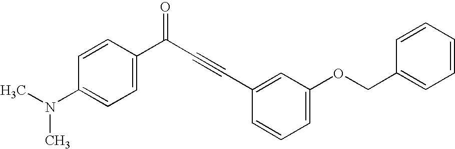 | Aventis Pharma | | EP 476658 |
| 482 | 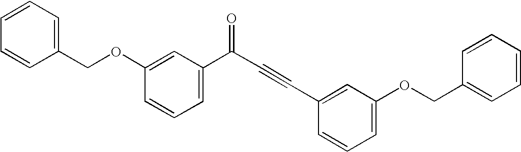 | Aventis Pharma | | EP 476658 |
| 483 | 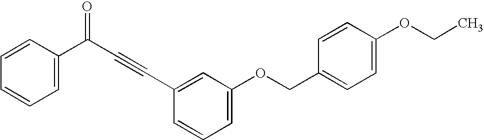 | Aventis Pharma | | EP 476658 |
| 484 | 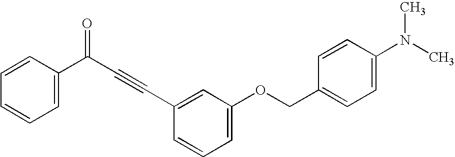 | Aventis Pharma | | EP 476658 |
| 485 | 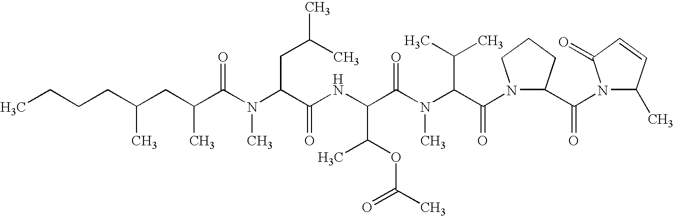 | Harbor Branch Found. | | US 5091368 |
| 486 | 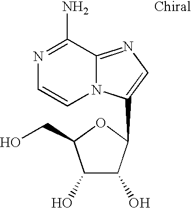 | Merck & Co. | | EP 480713 |
| 487 | 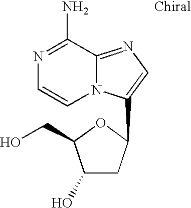 | Merck & Co. | | EP 480713 |
| 488 | 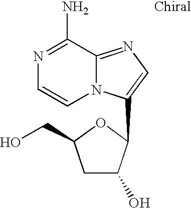 | Merck & Co. | | EP 480713 |
| 489 | 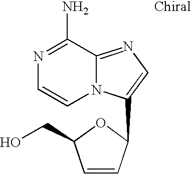 | Merck & Co. | | EP 480713 |
| 490 | 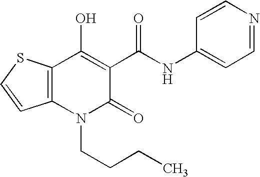 | Kyowa Hakko | Miwa, K. et al. 113th Annu Meet Pharmaceut Soc Jpn (March 29-31, Osaka) 1993, Abst 30CC 13-1. | EP 505058 |
| 491 | 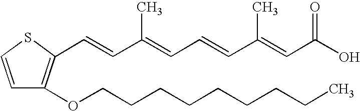 | Roche | | EP 510473 |
| 492 | 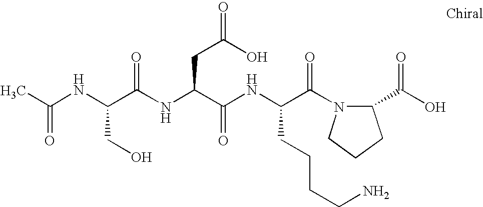 | Beaufour-Ipsen | 1) Carde, P. et al. Proc Amer Soc Clin Oncol 1991, 10: Abst 324. | AU 8810261 |
| 493 | 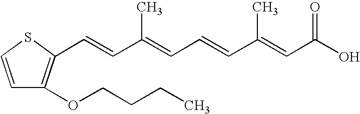 | Roche | | EP 510473 |
| 494 | 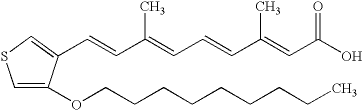 | Roche | | EP 510473 |
| 495 | 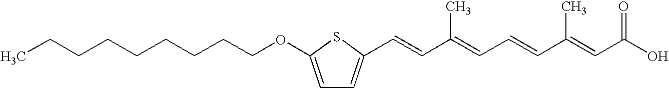 | Roche | | EP 510473 |
| 496 | 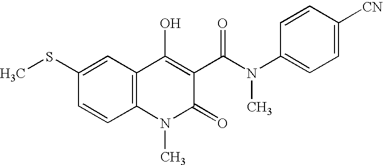 | Fujisawa | | WO 9218483 |
| 497 | 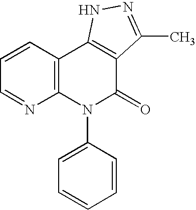 | Kyowa Hakko | | EP 526840 |
| 498 | 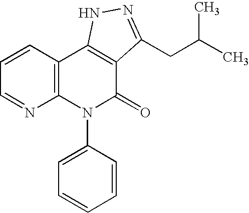 | Kyowa Hakko | | EP 526840 |
| 499 | 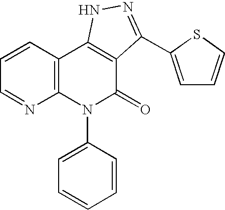 | Kyowa Hakko | | EP 526840 |
| 500 | 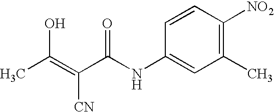 | Aventis Pharma | | EP 538783 |
| 501 | 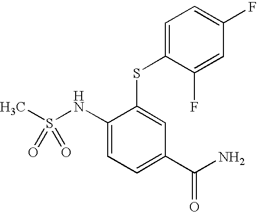 | Fujisawa Nakamura, K. et al. Chem Pharm Bull 1993, 41(5): 894. | AU 8783152 | |
| 502 | 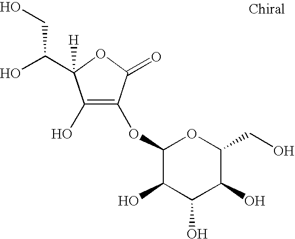 | Hayashibara | 1) Yamamoto, I. et al. 18th Int Cong Chemother (June 27-July 2, Stockholm) 1993, Abst 516. | EP 539196 |
| 503 | 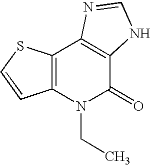 | Kyowa Hakko | | WO 9312116 |
| 504 | 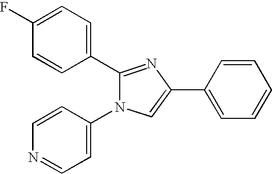 | GlaxoSmithKline | | WO 9314082 |
| 505 | 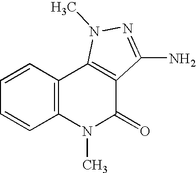 | Otsuka | | JP 93132484 |
| 506 | 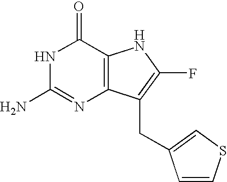 | Pfizer | | US 5236926 |
| 507 | 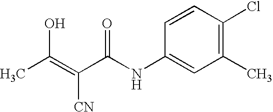 | Aventis Pharma | | EP 538783 |
| 508 | 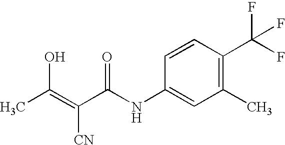 | Aventis Pharma | | EP 538783 |
| 509 | 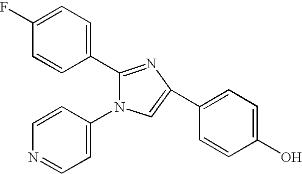 | GlaxoSmithKline | | WO 9314082 |
| 510 | 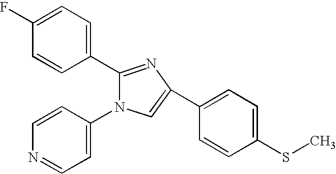 | GlaxoSmithKline | | WO 9314082 |
| 511 | 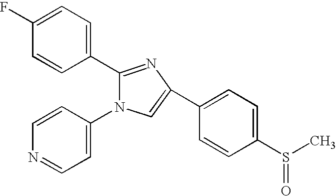 | GlaxoSmithKline | | WO 9314082 |
| 512 | 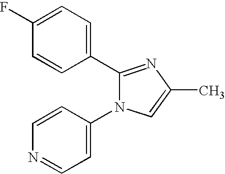 | GlaxoSmithKline | | WO 9314082 |
| 513 | 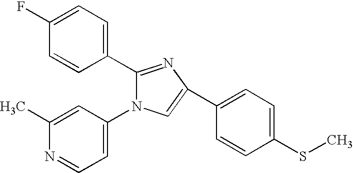 | GlaxoSmithKline | | WO 9314082 |
| 514 | 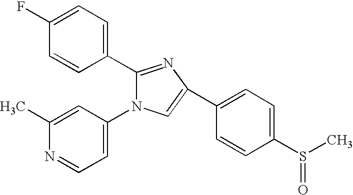 | GlaxoSmithKline | | WO 9314082 |
| 515 | 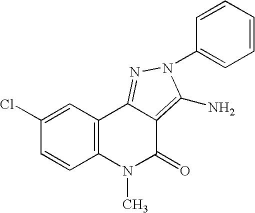 | Otsuka | | JP 93132484 |
| 516 | 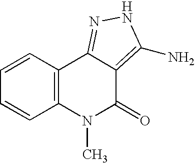 | Otsuka | | JP 93132484 |
| 517 | 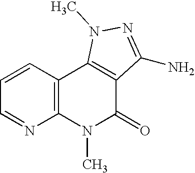 | Otsuka | | JP 93132484 |
| 518 | 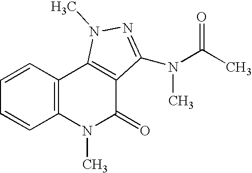 | Otsuka | | JP 93132484 |
| 519 | 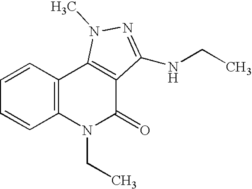 | Otsuka | | JP 93132484 |
| 520 | 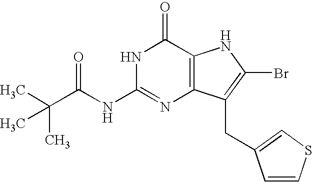 | Pfizer | | US 5236926 |
| 521 | 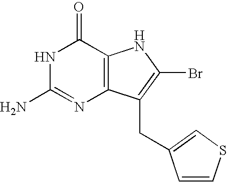 | Pfizer | | US 5236926 |
| 522 | 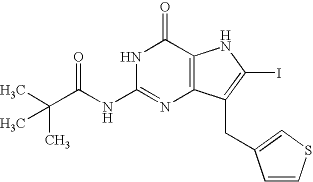 | Pfizer | | US 5236926 |
| 523 | 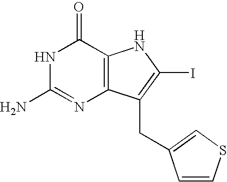 | Pfizer | | US 5236926 |
| 524 | 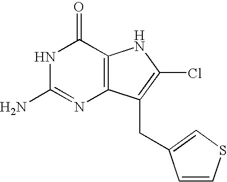 | Pfizer | | US 5236926 |
| 525 | 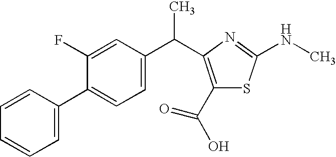 | Sumitomo | | US 5258396 |
| 526 | 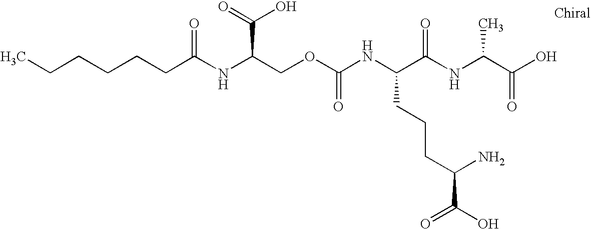 | Wyeth | | US 5312831 |
| 527 | 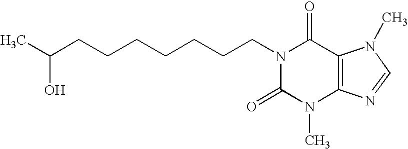 | Cell Therapeutics | | WO 9411001 |
| 528 | 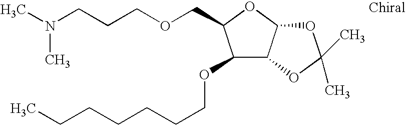 | Greenwich Pharm. | | WO 9411381 |
| 529 | 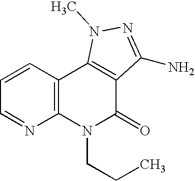 | Otsuka | | JP 94100561 |
| 530 | 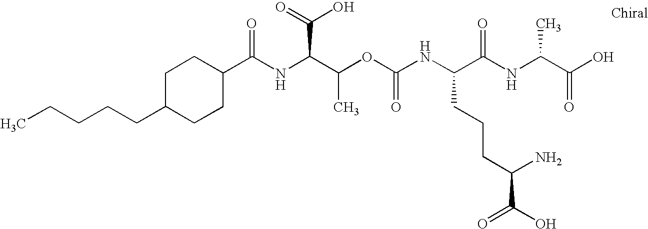 | Wyeth | | US 5312831 |
| 531 | 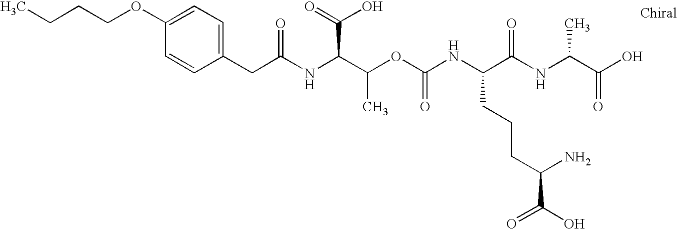 | Wyeth | | US 5312831 |
| 532 | 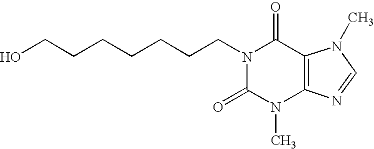 | Cell Therapeutics | | WO 9411001 |
| 533 | 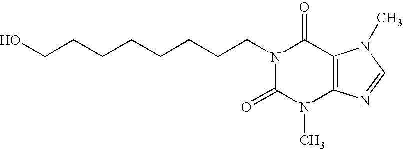 | Cell Therapeutics | | WO 9411001 |
| 534 | 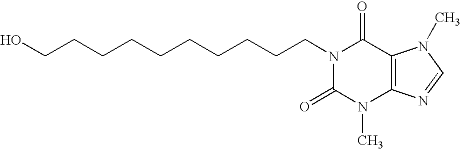 | Cell Therapeutics | | WO 9411001 |
| 535 | 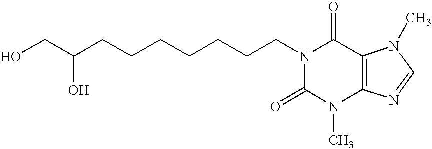 | Cell Therapeutics | | WO 9411001 |
| 536 | 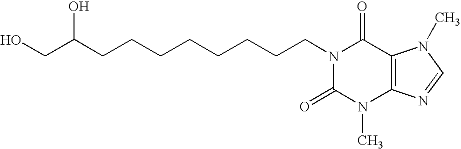 | Cell Therapeutics | | WO 9411001 |
| 537 | 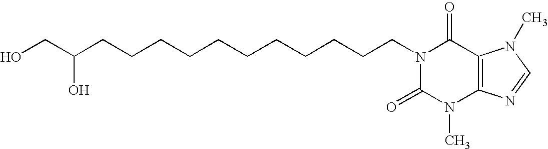 | Cell Therapeutics | | WO 9411001 |
| 538 | 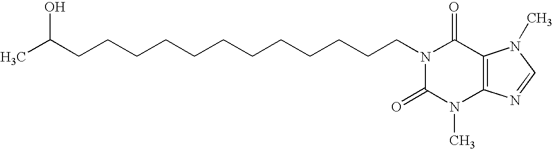 | Cell Therapeutics | | WO 9411001 |
| 539 | 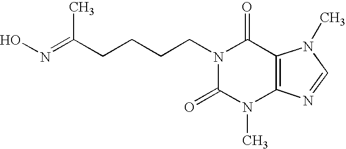 | Cell Therapeutics | | WO 9416704 |
| 540 | 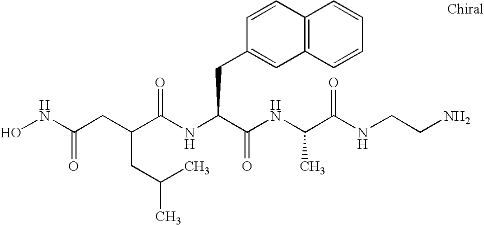 | Immunex | 1) Immunex Corporation Press Release 1994, July 21. | WO 9506031 |
| 541 | 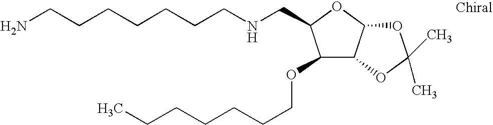 | Greenwich Pharm. | | WO 9411381 |
| 542 | 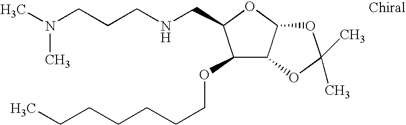 | Greenwich Pharm. | | WO 9411381 |
| 543 | 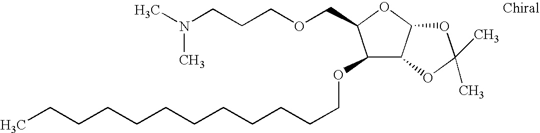 | Greenwich Pharm. | | WO 9411381 |
| 544 | 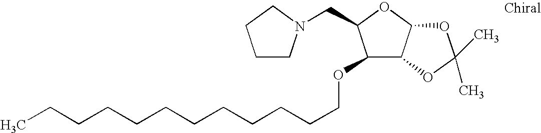 | Greenwich Pharm. | | WO 9411381 |
| 545 | 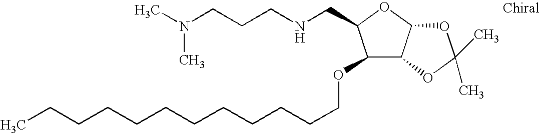 | Greenwich Pharm. | | WO 9411381 |
| 546 | 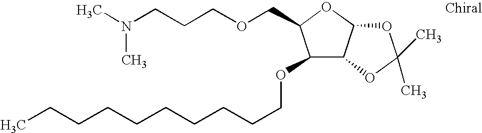 | Greenwich Pharm. | | WO 9411381 |
| 547 | 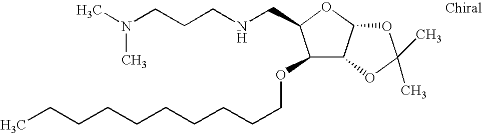 | Greenwich Pharm. | | WO 9411381 |
| 548 | 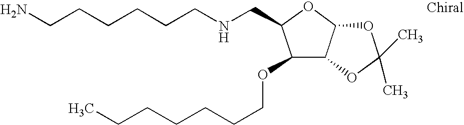 | Greenwich Pharm. | | WO 9411381 |
| 549 | 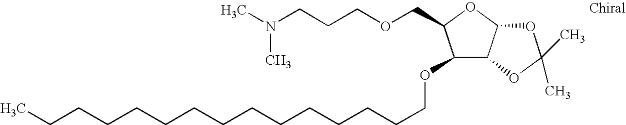 | Greenwich Pharm. | | WO 9411381 |
| 550 | 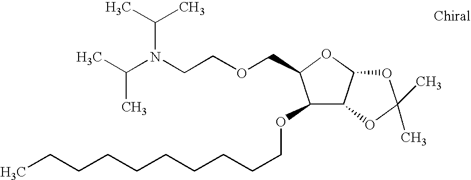 | Greenwich Pharm. | | WO 9411381 |
| 551 | 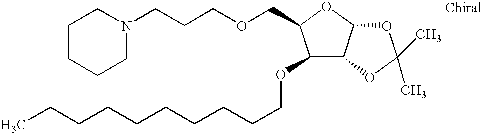 | Greenwich Pharm. | | WO 9411381 |
| 552 | 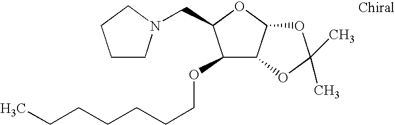 | Greenwich Pharm. | | WO 9411381 |
| 553 | 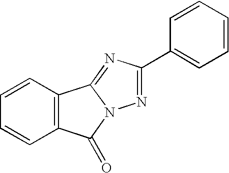 | British Technol. | | GB 2278842 |
| 554 | 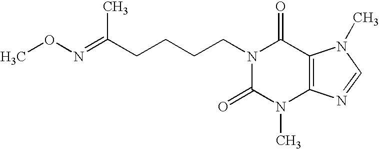 | Cell Therapeutics | | WO 9416704 |
| 555 | 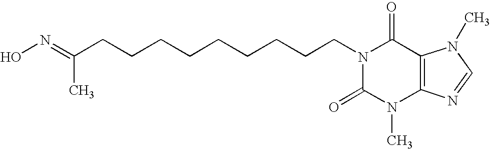 | Cell Therapeutics | | WO 9416704 |
| 556 | 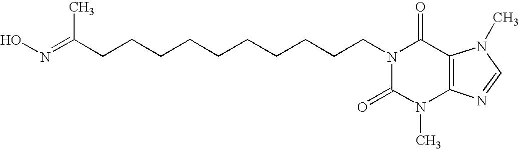 | Cell Therapeutics | | WO 9416704 |
| 557 | 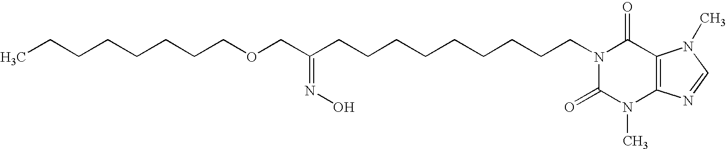 | Cell Therapeutics | | WO 9416704 |
| 558 | 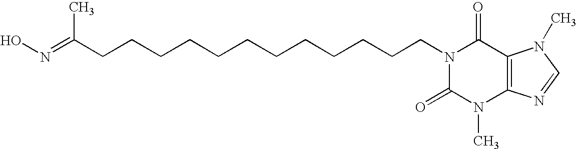 | Cell Therapeutics | | WO 9416704 |
| 559 | 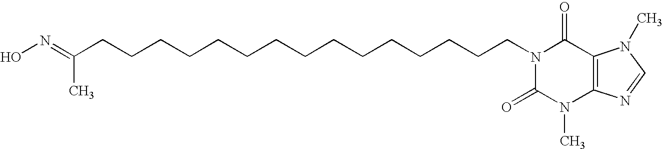 | Cell Therapeutics | | WO 9416704 |
| 560 | 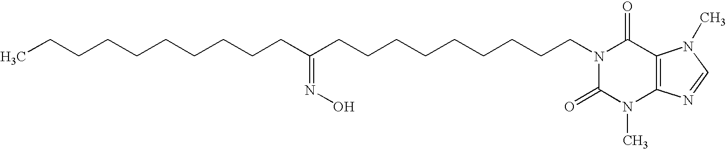 | Cell Therapeutics | | WO 9416704 |
| 561 | 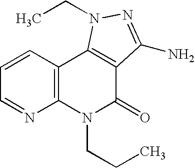 | Otsuka | | JP 94100561 |
| 562 | 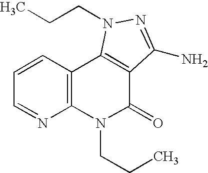 | Otsuka | | JP 94100561 |
| 563 | 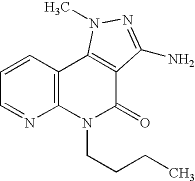 | Otsuka | | JP 94100561 |
| 564 | 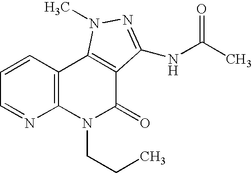 | Otsuka | | JP 94100561 |
| 565 | 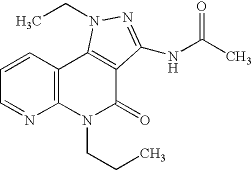 | Otsuka | | JP 94100561 |
| 566 | 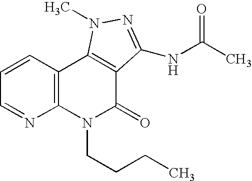 | Otsuka | | JP 94100561 |
| 567 | 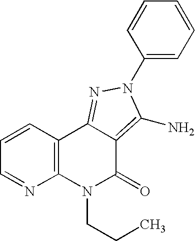 | Otsuka | | JP 94100561 |
| 568 | 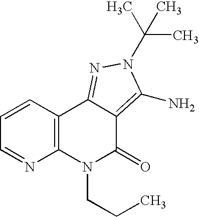 | Otsuka | | JP 94100561 |
| 569 | 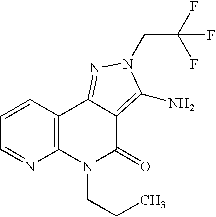 | Otsuka | | JP 94100561 |
| 570 | 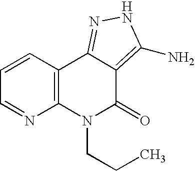 | Otsuka | | JP 94100561 |
| 571 | 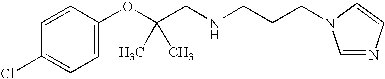 | Abbott GmbH | | WO 9500493 |
| 572 | 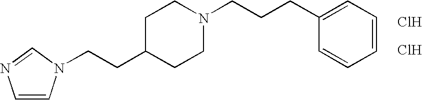 | Abbott GmbH | | WO 9500507 |
| 573 | 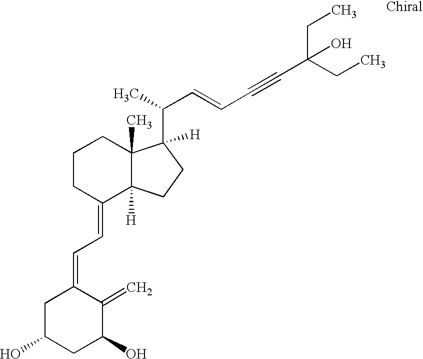 | Leo | | WO 9502577 |
| 574 | 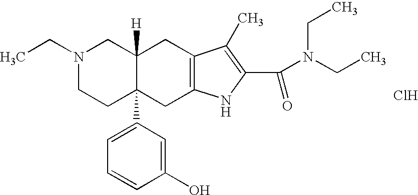 | GlaxoSmithKline | | WO 9504734 |
| 575 | 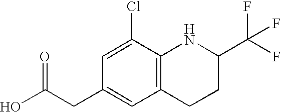 | Kyorin | Kono, Y. et al. 115th Annu Meet Pharmaceut Soc Jpn (March 29-31, Sendai) 1995, Abst 30 (A3) 10-3. | EP 538477 |
| 576 | 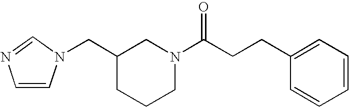 | Abbott GmbH | | WO 9500507 |
| 577 | 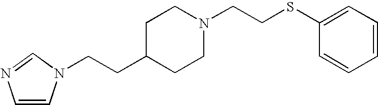 | Abbott GmbH | | WO 9500507 |
| 578 | 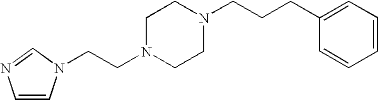 | Abbott GmbH | | WO 9500507 |
| 579 | 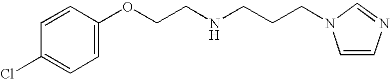 | Abbott GmbH | | WO 9500493 |
| 580 | 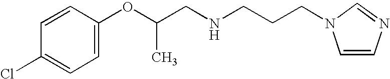 | Abbott GmbH | | WO 9500493 |
| 581 | 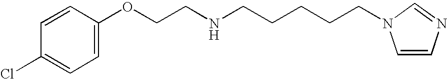 | Abbott GmbH | | WO 9500493 |
| 582 | 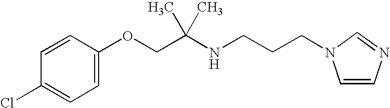 | Abbott GmbH | | WO 9500493 |
| 583 | 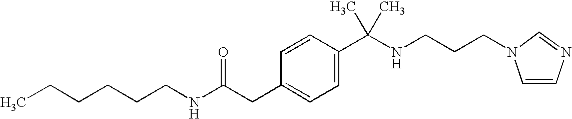 | Abbott GmbH | | WO 9500493 |
| 584 | 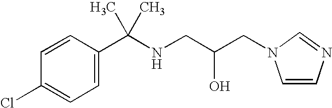 | Abbott GmbH | | WO 9500493 |
| 585 | 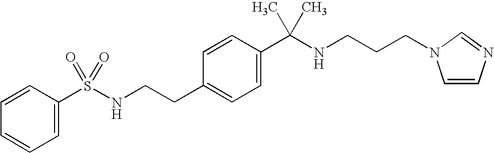 | Abbott GmbH | | WO 9500493 |
| 586 | 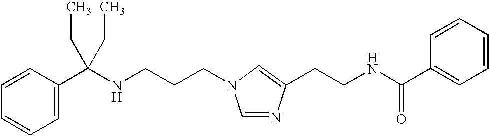 | Abbott GmbH | | WO 9500493 |
| 587 | 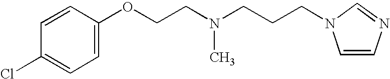 | Abbott GmbH | | WO 9500493 |
| 588 | 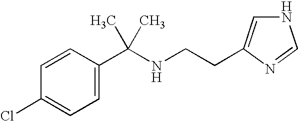 | Abbott GmbH | | WO 9500493 |
| 589 | 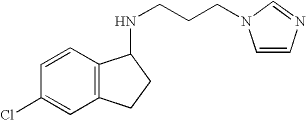 | Abbott GmbH | | WO 9500493 |
| 590 | 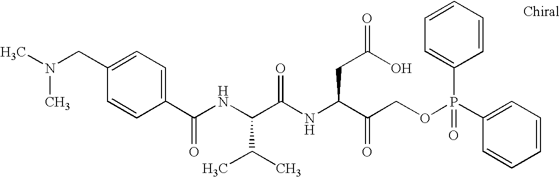 | Sanofi- Synthelabo | | EP 644197 |
| 591 | 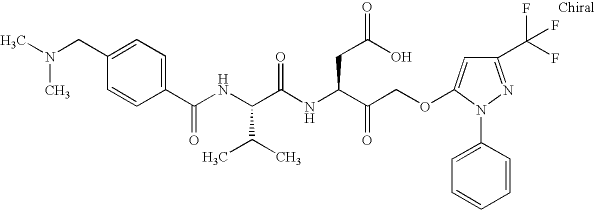 | Sanofi- Synthelabo | | CA 2125021 |
| 592 | 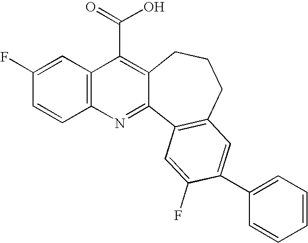 | Kyowa Hakko | | WO 9509153 |
| 593 | 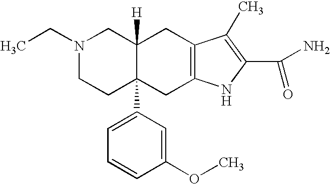 | GlaxoSmithKline | | WO 9504734 |
| 594 | 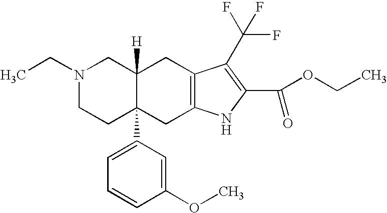 | GlaxoSmithKline | | WO 9504734 |
| 595 | 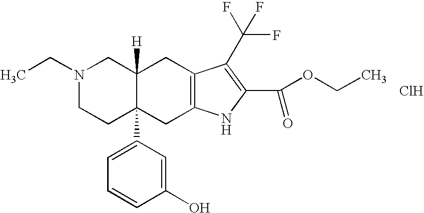 | GlaxoSmithKline | | WO 9504734 |
| 596 | 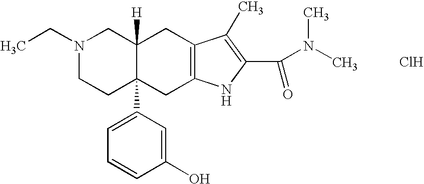 | GlaxoSmithKline | | WO 9504734 |
| 597 | 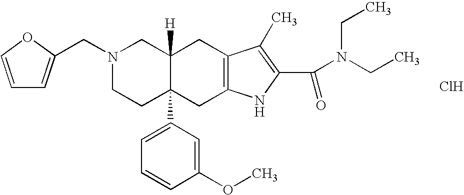 | GlaxoSmithKline | | WO 9504734 |
| 598 | 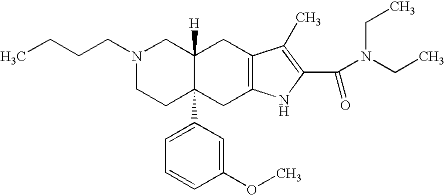 | GlaxoSmithKline | | WO 9504734 |
| 599 | 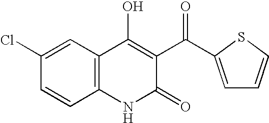 | Japan Tobacco | | JP 95002779 |
| 600 | 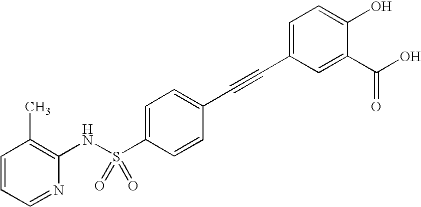 | Pharmacia | 1) Gozzi, P. et al. J Pharmacol Exp Ther 1999, 291(1): 199. | JP 1995501330 |
| 601 | 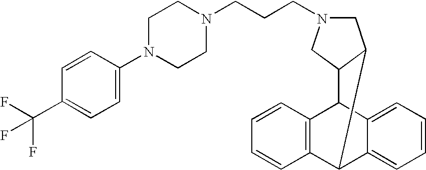 | Bayer | | US 5409932 |
| 602 | 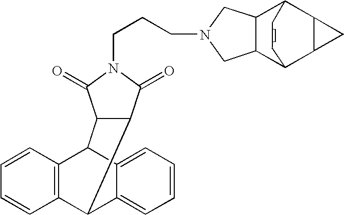 | Bayer | | US 5411960 |
| 603 | 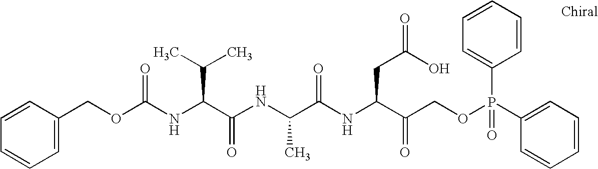 | Sanofi- Synthelabo | | EP 644197 |
| 604 | 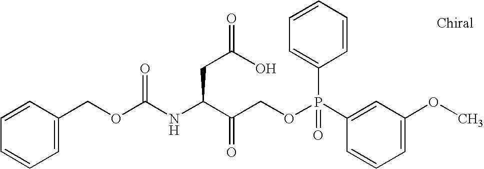 | Sanofi- Synthelabo | | EP 644197 |
| 605 | 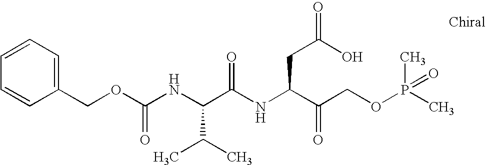 | Sanofi- Synthelabo | | EP 644197 |
| 606 | 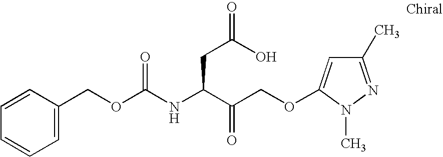 | Sanofi- Synthelabo | | CA 2125021 |
| 607 | 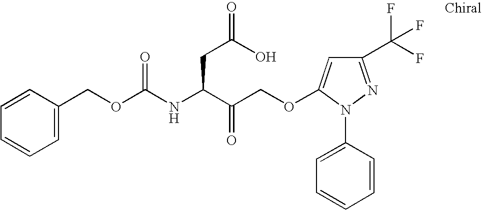 | Sanofi- Synthelabo | | CA 2125021 |
| 608 | 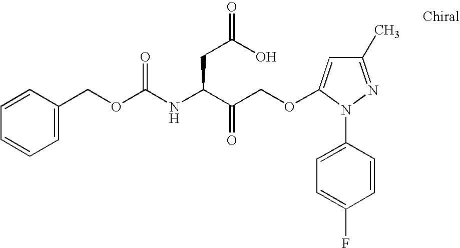 | Sanofi- Synthelabo | | CA 2125021 |
| 609 | 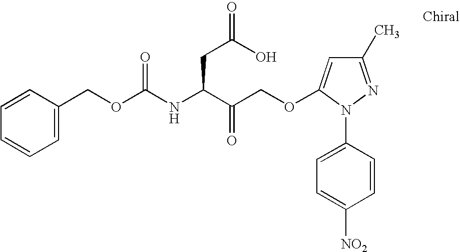 | Sanofi- Synthelabo | | CA 2125021 |
| 610 | 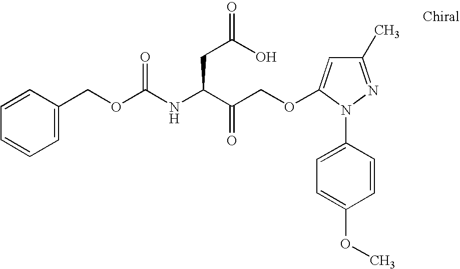 | Sanofi- Synthelabo | | CA 2125021 |
| 611 | 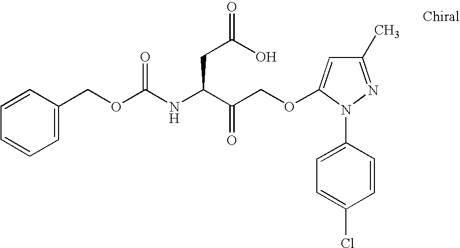 | Sanofi- Synthelabo | | CA 2125021 |
| 612 | 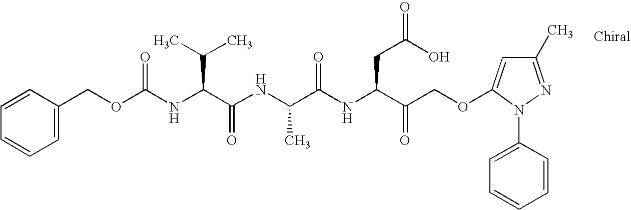 | Sanofi- Synthelabo | | CA 2125021 |
| 613 | 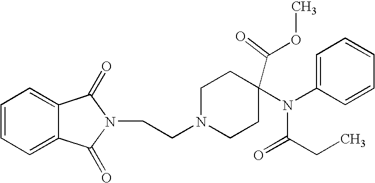 | Baxter | France, C. P. et al. Drug Develop Res 1995, 35: 49. | EP 396282 |
| 614 | 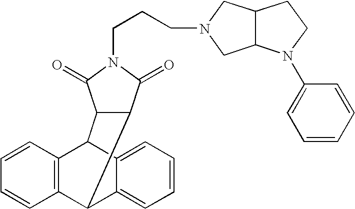 | Bayer | | U5 5411960 |
| 615 | 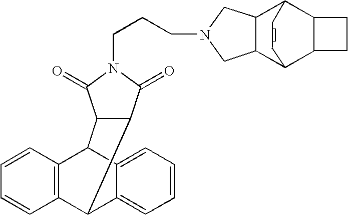 | Bayer | | US 5411960 |
| 616 | 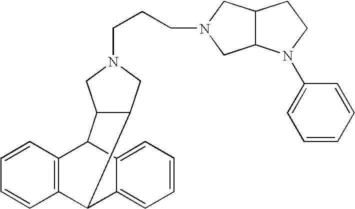 | Bayer | | US 5411960 |
| 617 | 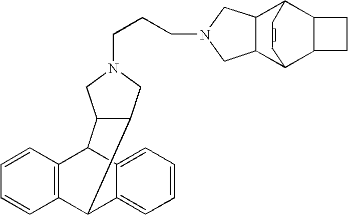 | Bayer | | US 5411960 |
| 618 | 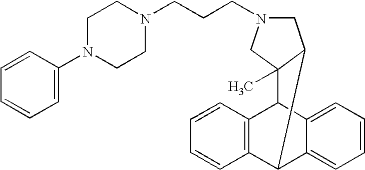 | Bayer | | US 5409932 |
| 619 | 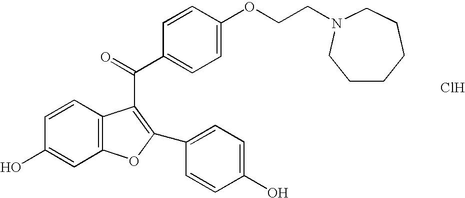 | Lilly | | WO 9517382 |
| 620 | 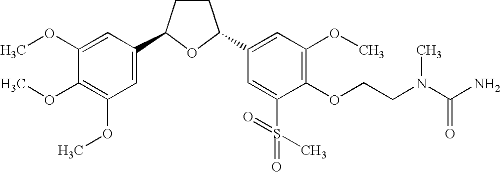 | Millennium | | WO 9518610 |
| 621 | 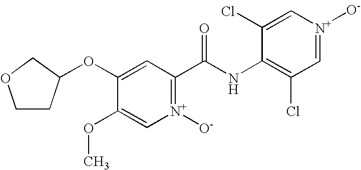 | Aventis Pharma | | WO 9520578 |
| 622 | 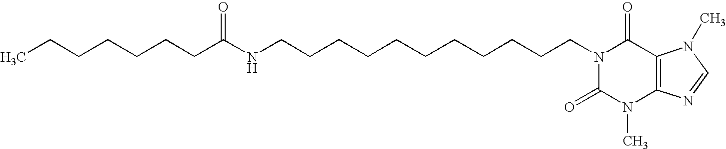 | Cell Therapeutics | | WO 9522546 |
| 623 | 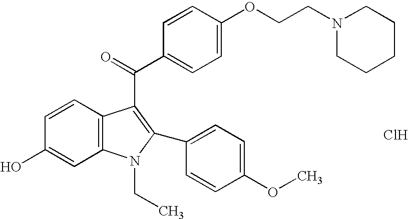 | Lilly | | WO 9517382 |
| 624 | 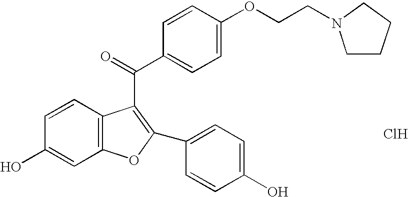 | Lilly | | WO 9517382 |
| 625 | 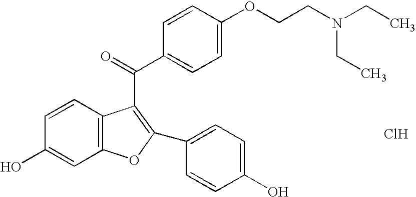 | Lilly | | WO 9517382 |
| 626 | 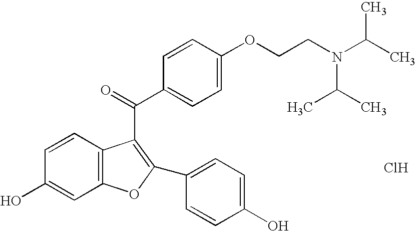 | Lilly | | WO 9517382 |
| 627 | 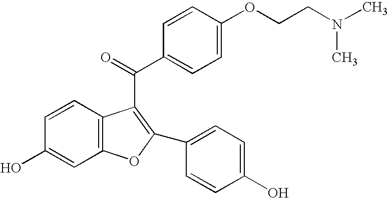 | Lilly | | WO 9517382 |
| 628 | 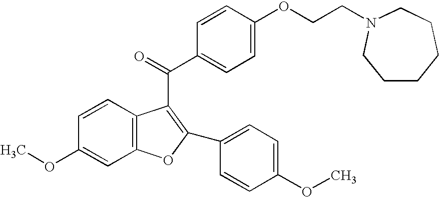 | Lilly | | WO 9517382 |
| 629 | 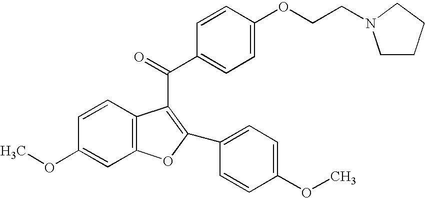 | Lilly | | WO 9517382 |
| 630 | 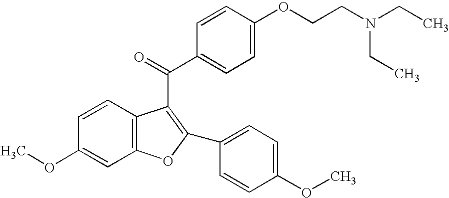 | Lilly | | WO 9517382 |
| 631 | 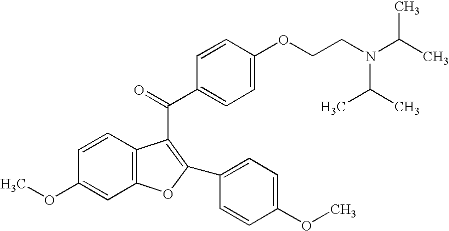 | Lilly | | WO 9517382 |
| 632 | 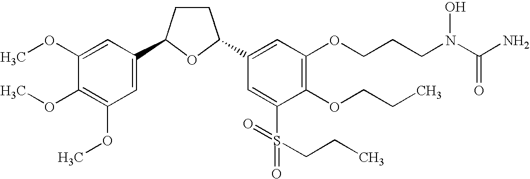 | CytoMed | | WO 9518610 |
| 633 | 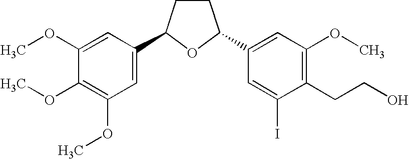 | CytoMed | | WO 9518610 |
| 634 | 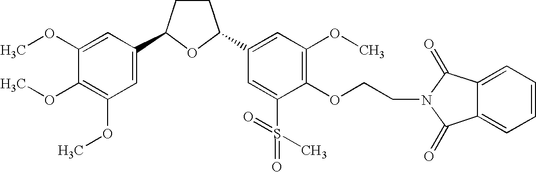 | CytoMed | | WO 9518610 |
| 635 | 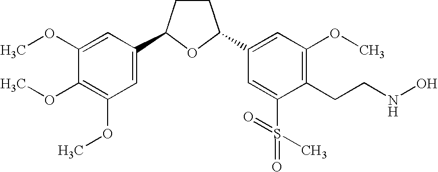 | CytoMed | | WO 9518610 |
| 636 | 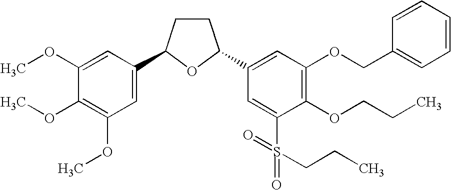 | CytoMed | | WO 9518610 |
| 637 | 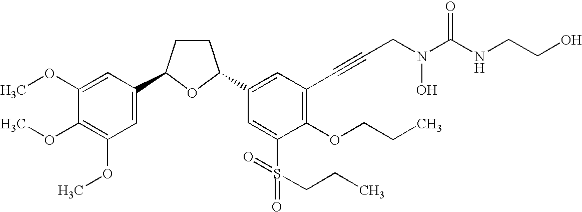 | CytoMed | | WO 9518610 |
| 638 | 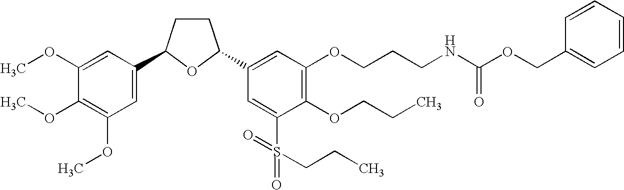 | CytoMed | | WO 9518610 |
| 639 | 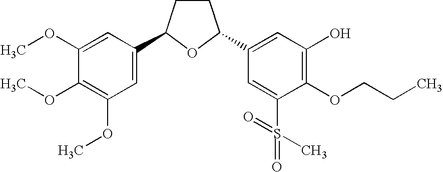 | CytoMed | | WO 9518610 |
| 640 | 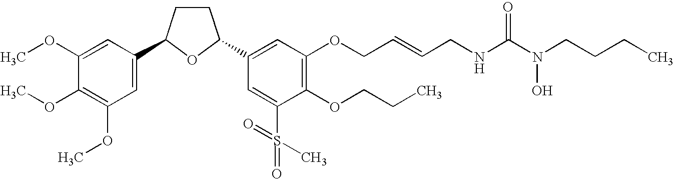 | CytoMed | | WO 9518610 |
| 641 | 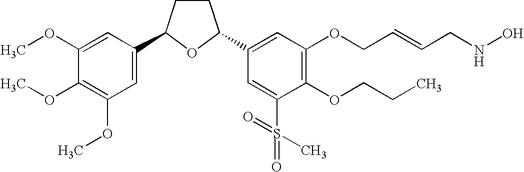 | CytoMed | | WO 9518610 |
| 642 | 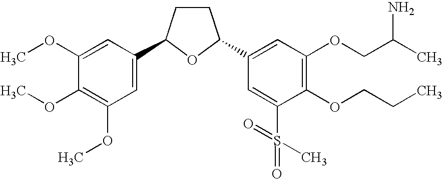 | CytoMed | | WO 9518610 |
| 643 | 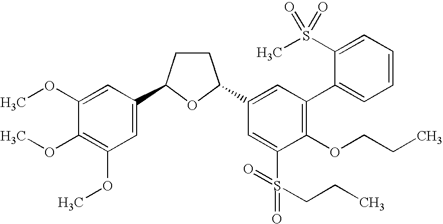 | CytoMed | | WO 9518610 |
| 644 | 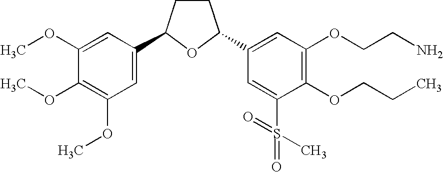 | CytoMed | | WO 9518610 |
| 645 | 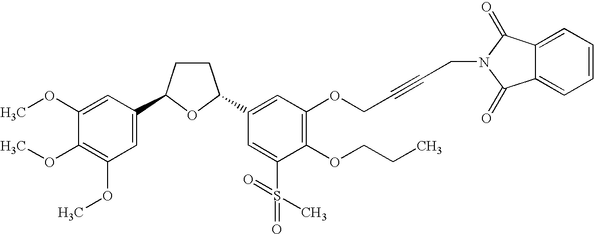 | CytoMed | | WO 9518610 |
| 646 | 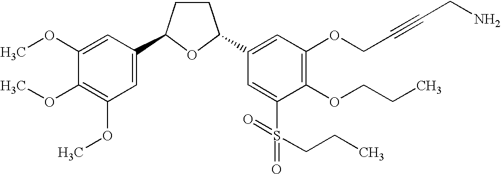 | CytoMed | | WO 9518610 |
| 647 | 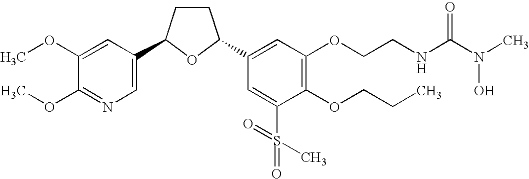 | CytoMed | | WO 9518610 |
| 648 | 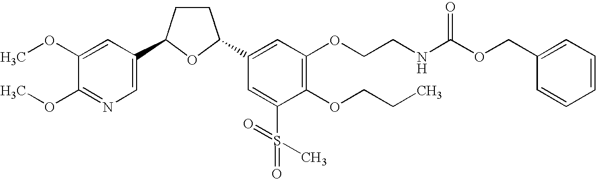 | CytoMed | | WO 9518610 |
| 649 | 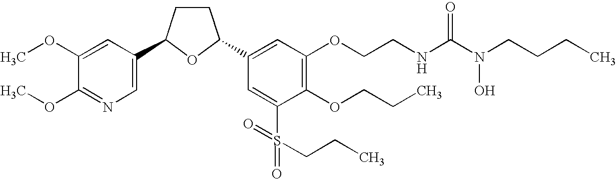 | CytoMed | | WO 9518610 |
| 650 | 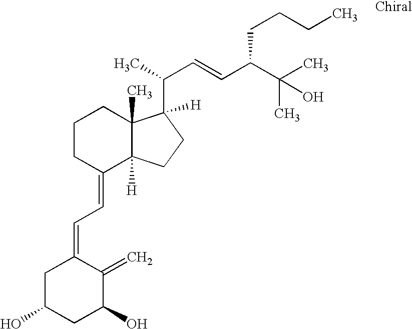 | Duphar | | EP 664287 |
| 651 | 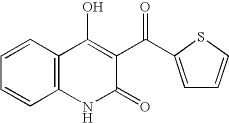 | Japan Tobacco | | JP 95002779 |
| 652 | 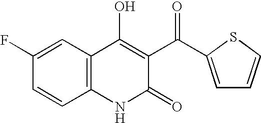 | Japan Tobacco | | JP 95002779 |
| 653 | 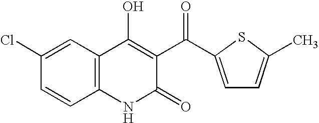 | Japan Tobacco | | JP 95002779 |
| 654 | 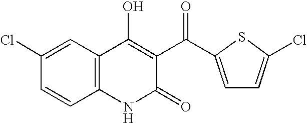 | Japan Tobacco | | JP 95002779 |
| 655 | 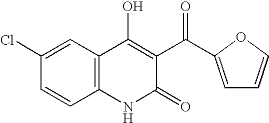 | Japan Tobacco | | JP 95002779 |
| 656 | 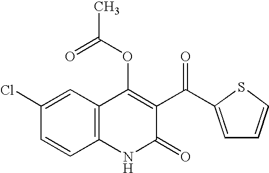 | Japan Tobacco | | JP 95002779 |
| 657 | 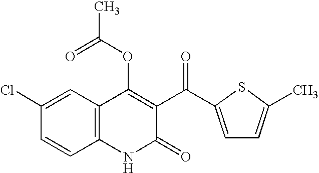 | Japan Tobacco | | JP 95002779 |
| 658 | 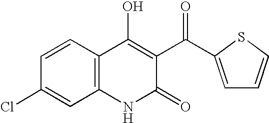 | Japan Tobacco | | JP 95002779 |
| 659 | 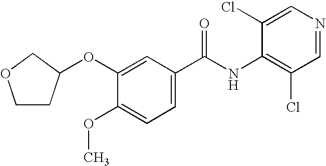 | Aventis Pharma | | WO 9520578 |
| 660 | 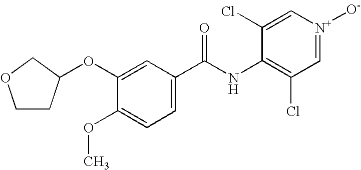 | Aventis Pharma | | WO 9520578 |
| 661 | 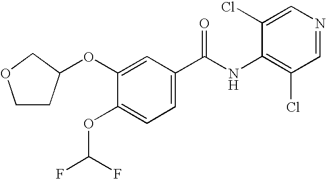 | Aventis Pharma | | WO 9520578 |
| 662 | 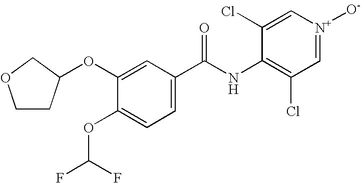 | Aventis Pharma | | WO 9520578 |
| 663 | 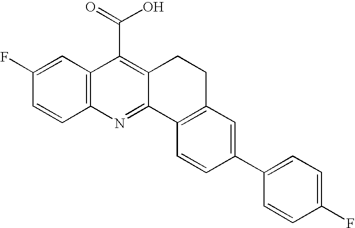 | Kyowa Hakko | | WO 9509153 |
| 664 | 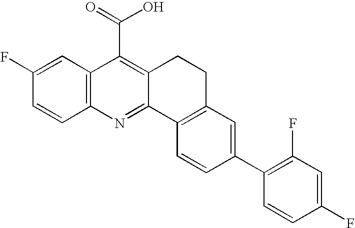 | Kyowa Hakko | | WO 9509153 |
| 665 | 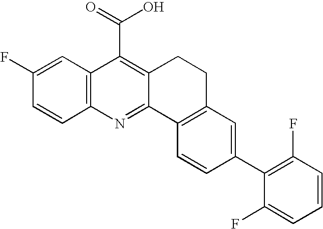 | Kyowa Hakko | | WO 9509153 |
| 666 | 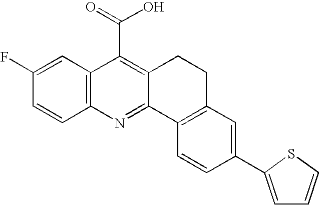 | Kyowa Hakko | | WO 9509153 |
| 667 | 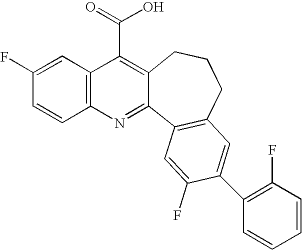 | Kyowa Hakko | | WO 9509153 |
| 668 | 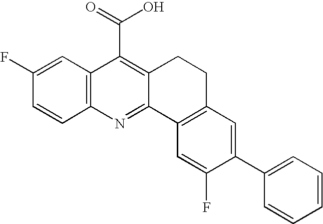 | Kyowa Hakko | | WO 9509153 |
| 669 | 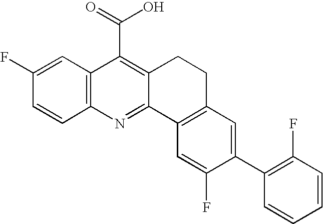 | Kyowa Hakko | | WO 9509153 |
| 670 | 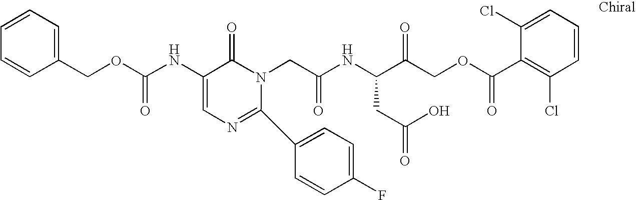 | Sanofi- Synthelabo | | WO 9526958 |
| 671 | 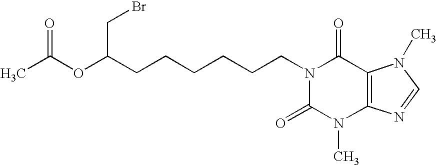 | Cell Therapeutics | | WO 9522546 |
| 672 | 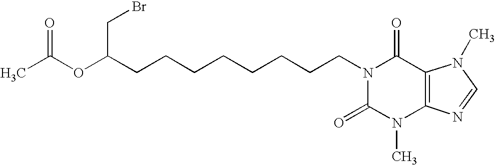 | Cell Therapeutics | | WO 9522546 |
| 673 | 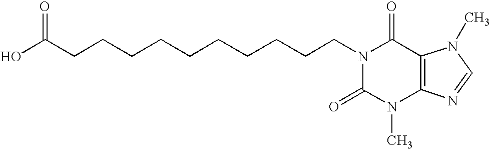 | Cell Therapeutics | | WO 9522546 |
| 674 | 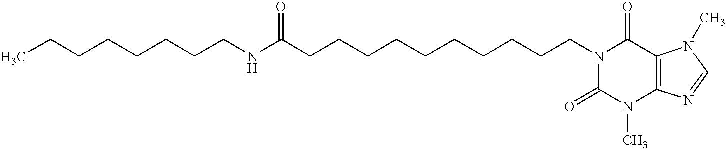 | Cell Therapeutics | | WO 9522546 |
| 675 | 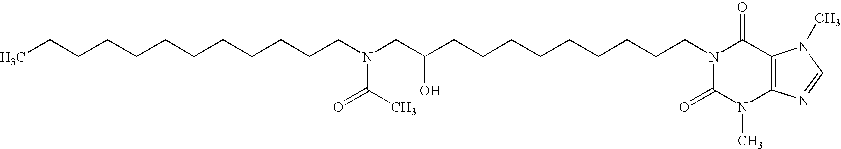 | Cell Therapeutics | | WO 9522546 |
| 676 | 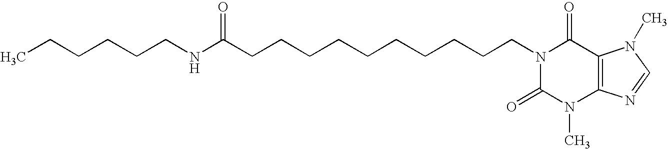 | Cell Therapeutics | | WO 9522546 |
| 677 | 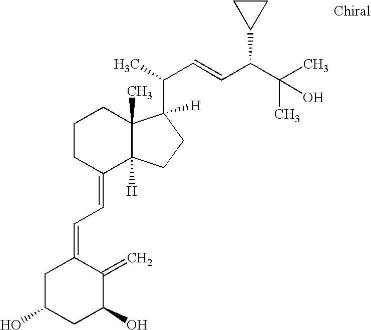 | Duphar | | EP 664287 |
| 678 | 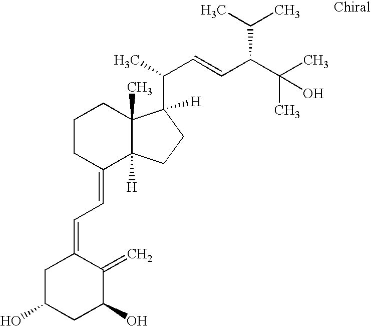 | Duphar | | EP 664287 |
| 679 | 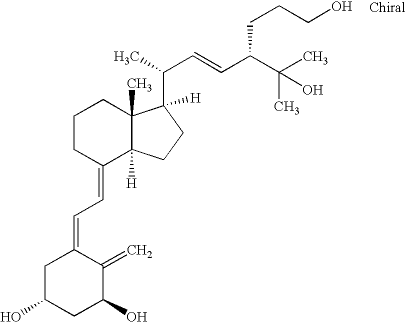 | Duphar | | EP 664287 |
| 680 | 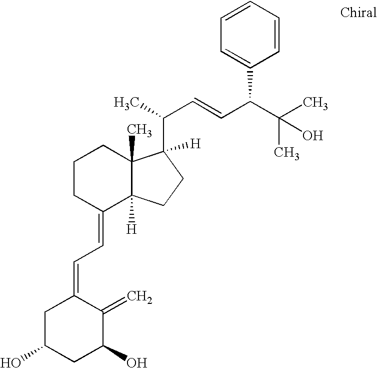 | Duphar | | EP 664287 |
| 681 | 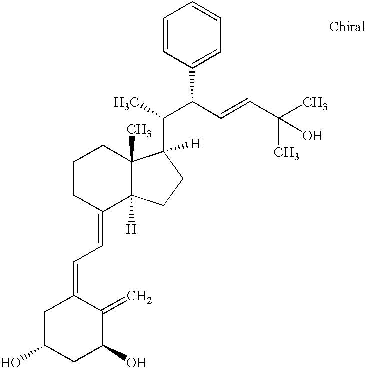 | Duphar | | EP 664287 |
| 682 | 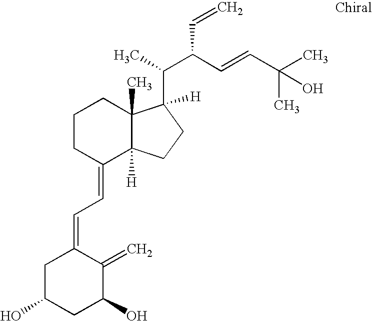 | Duphar | | EP 664287 |
| 683 | 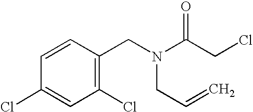 | Sanofi- Synthelabo | | WO 9529672 |
| 684 | 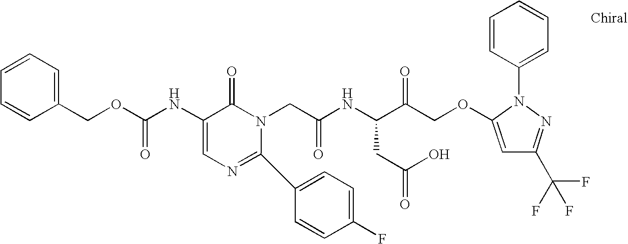 | Sanofi- Synthelabo | | WO 9526958 |
| 685 | 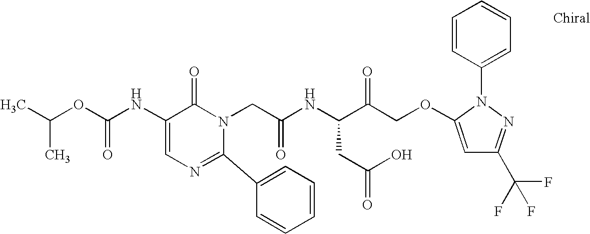 | Sanofi- Synthelabo | | WO 9526958 |
| 686 | 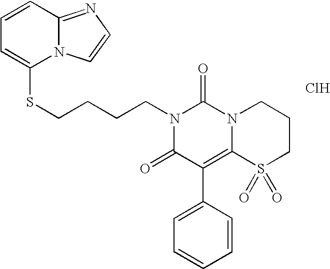 | Takeda | | WO 9535296 |
| 687 | 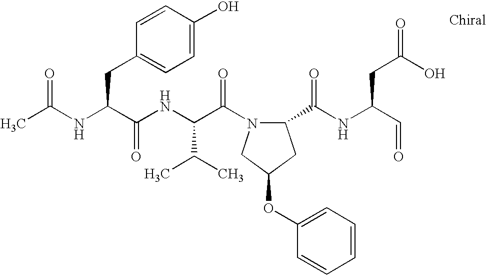 | Vertex | | WO 9535308 |
| 688 | 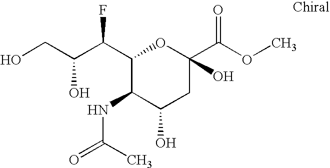 | Daikin | | EP 711766 |
| 689 | 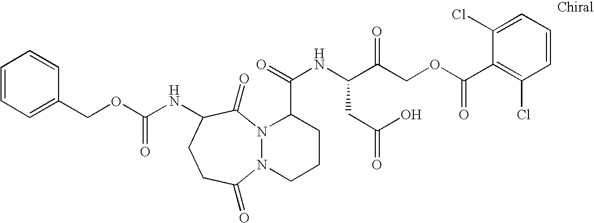 | Sanofi- Synthelabo | | WO 9533751 |
| 690 | 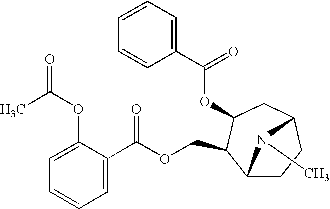 | Entropin | | WO 9534561 |
| 691 | 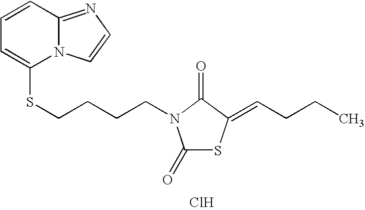 | Takeda | | WO 9535296 |
| 692 | 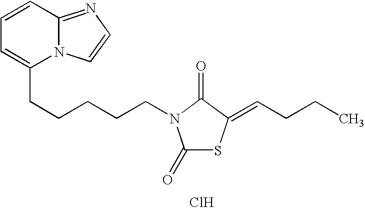 | Takeda | | WO 9535296 |
| 693 | 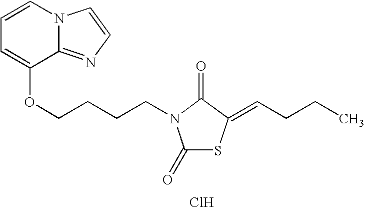 | Takeda | | WO 9535296 |
| 694 | 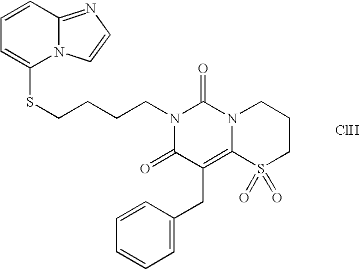 | Takeda | | WO 9535296 |
| 695 | 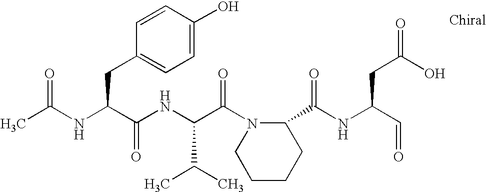 | Vertex | | WO 9535308 |
| 696 | 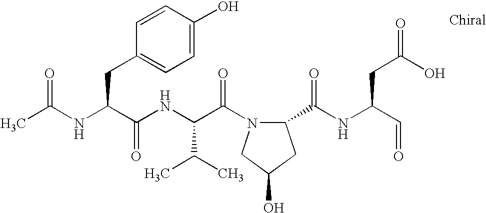 | Vertex | | WO 9535308 |
| 697 | 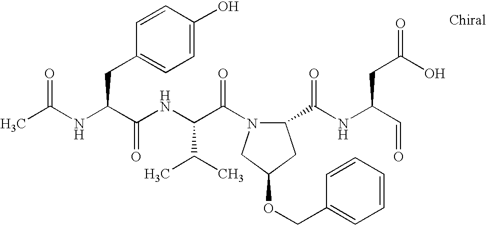 | Vertex | | WO 9535308 |
| 698 | 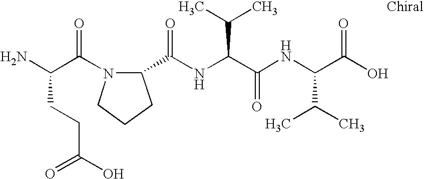 | Ajinomoto | | US 5464819 |
| 699 | 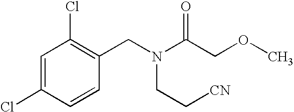 | Sanofi- Synthelabo | | WO 9529672 |
| 700 | 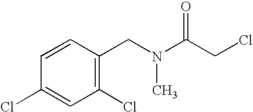 | Sanofi- Synthelabo | | WO 9529672 |
| 701 | 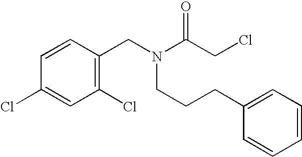 | Sanofi- Synthelabo | | WO 9529672 |
| 702 | 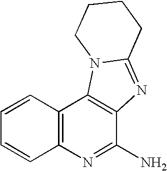 | 3M Pharmaceuticals | | US 5482936 |
| 703 | 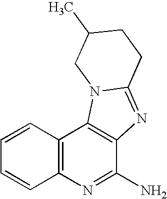 | 3M Pharmaceuticals | | US 5482936 |
| 704 | 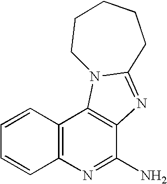 | 3M Pharmaceuticals | | US 5482936 |
| 705 | 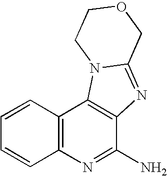 | 3M Pharmaceuticals | | US 5482936 |
| 706 | 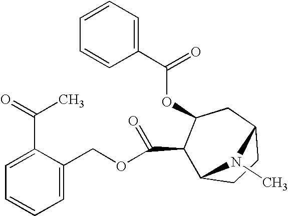 | Entropin | | WO 9534561 |
| 707 | 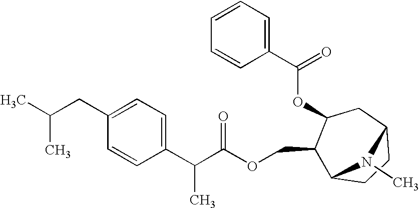 | Entropin | | WO 9534561 |
| 708 | 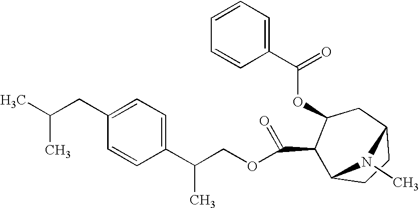 | Entropin | | WO 9534561 |
| 709 | 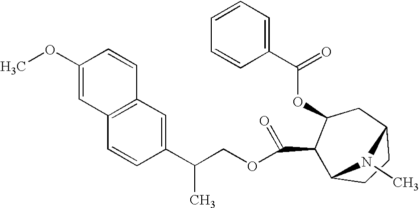 | Entropin | | WO 9534561 |
| 710 | 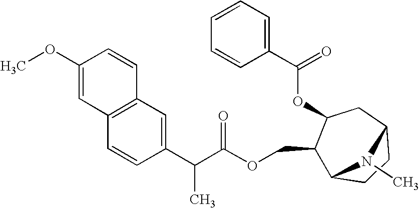 | Entropin | | WO 9534561 |
| 711 | 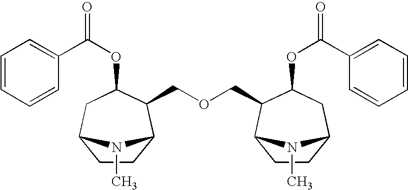 | Entropin | | WO 9534561 |
| 712 | 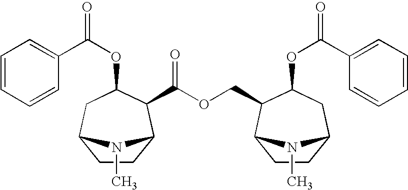 | Entropin | | WO 9534561 |
| 713 | 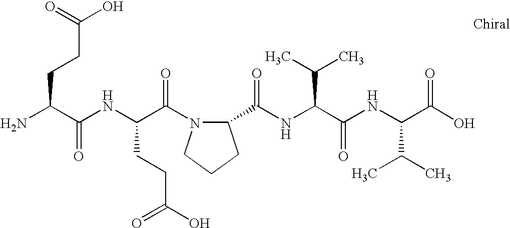 | Ajinomoto | | US 5464819 |
| 714 | 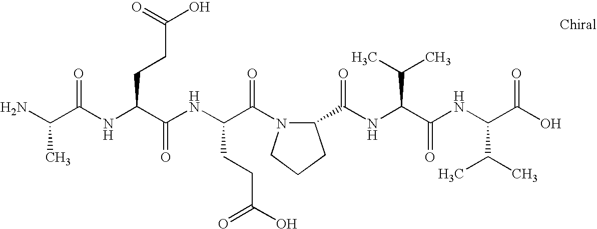 | Ajinomoto | | US 5464819 |
| 715 | 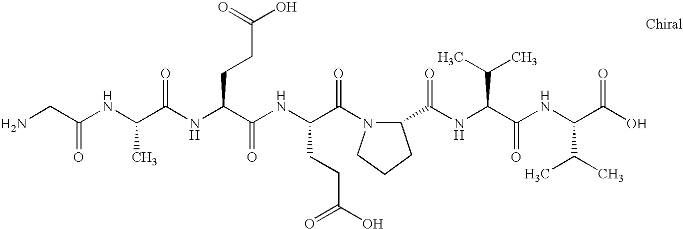 | Ajinomoto | | US 5464819 |
| 716 | 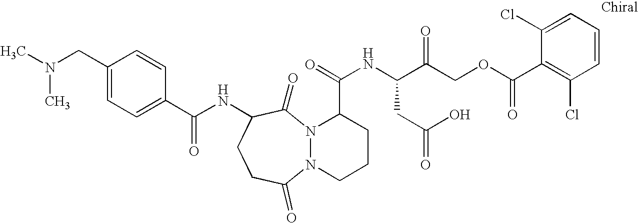 | Sanofi- Synthelabo | | WO 9533751 |
| 717 | 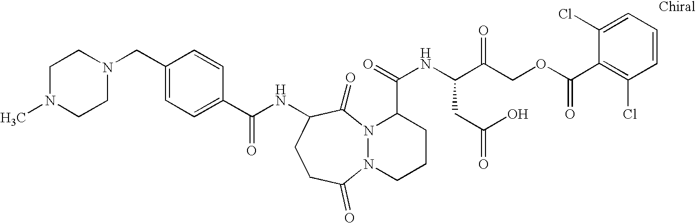 | Sanofi- Synthelabo | | WO 9533751 |
| 718 | 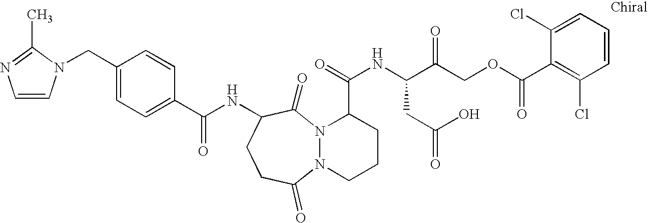 | Sanofi- Synthelabo | | WO 9533751 |
| 719 | 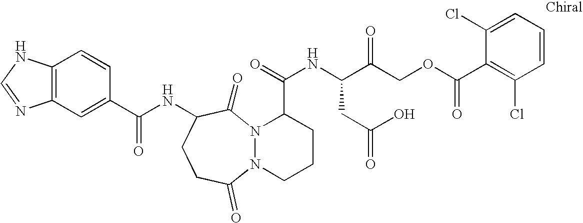 | Sanofi- Synthelabo | | WO 9533751 |
| 720 | 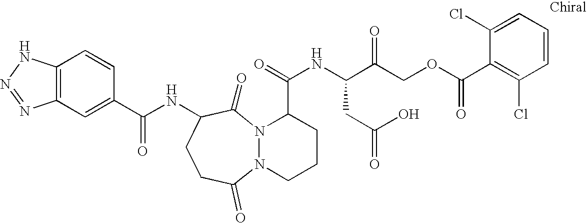 | Sanofi- Synthelabo | | WO 9533751 |
| 721 | 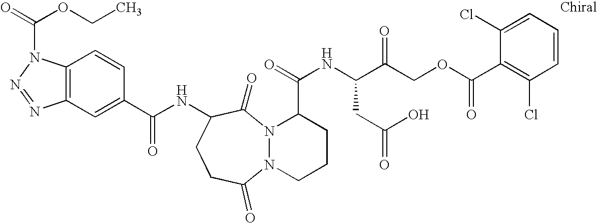 | Sanofi- Synthelabo | | WO 9533751 |
| 722 | 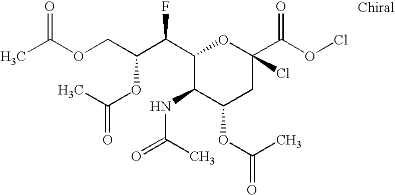 | Daikin | | EP 711766 |
| 723 | 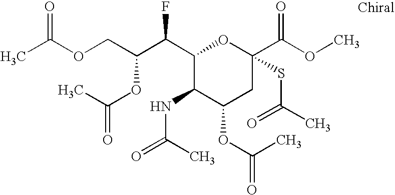 | Daikin | | EP 711766 |
| 724 | 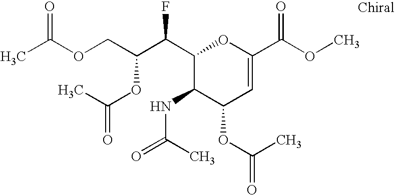 | Daikin | | EP 711766 |
| 725 | 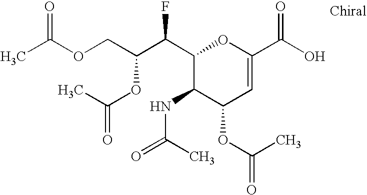 | Daikin | | EP 711766 |
| 726 | 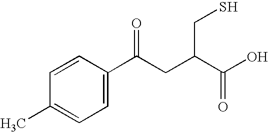 | Taisho | 1) Yoshida, H. et al. Biol Pharm Bull 1997, 20(1): 94. | JP 93051358 |
| 727 | 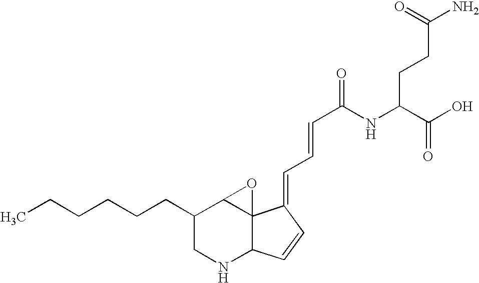 | Microbial Chemistry Research Foundation | | JP 96176157 |

| | | | | |
|---|---|---|---|---|
| 728 | 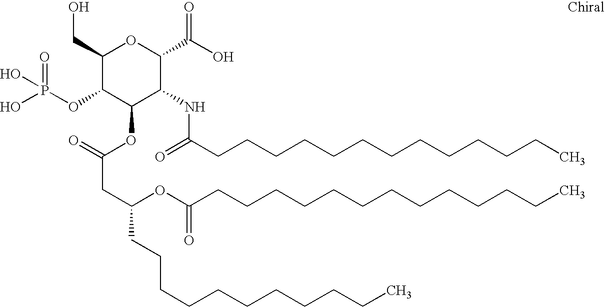 | Sankyo | Shiozaki, M. et al. Tetrahedron Lett 1996 37(40): 7271. | |
| 729 | 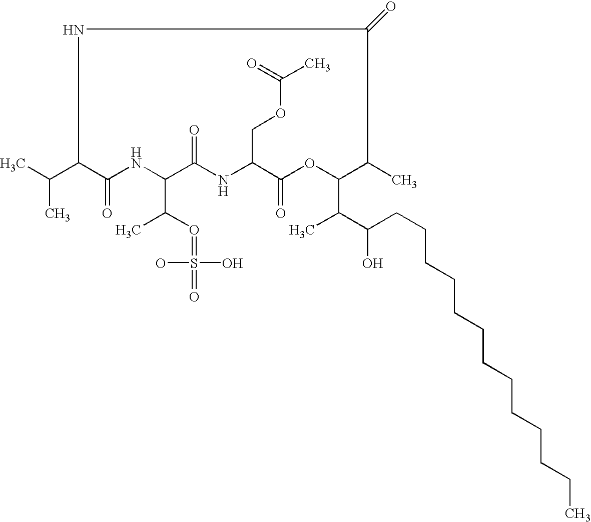 | Nippon Kayaku | | JP 96283290 |
| 730 | 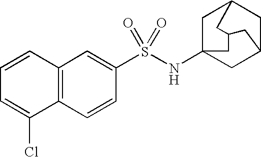 | Tanabe | | WO 9640641 |
| 731 | 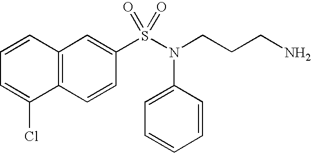 | Tanabe | | WO 9640641 |
| 732 | 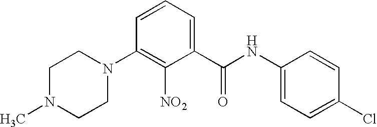 | Daiichi Pharma- ceutical | 1) Kawagoe, K. et al. AFMC Int Med Chem Symp (Sept 3-8, Tokyo) 1995, Abst P13M183. | JP 97059236 |
| 733 | 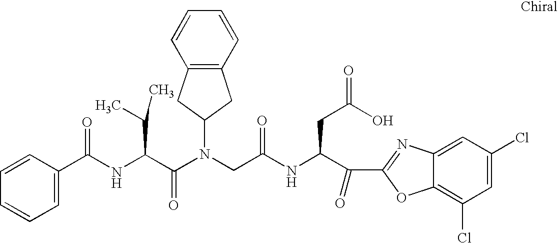 | Vertex | | WO 9722618 |
| 734 | 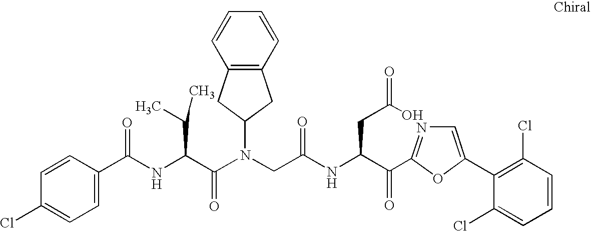 | Vertex | | WO 9722618 |
| 735 | 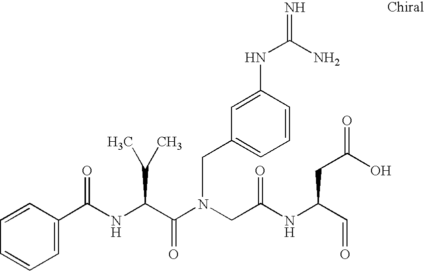 | Vertex | | WO 9722618 |
| 736 | 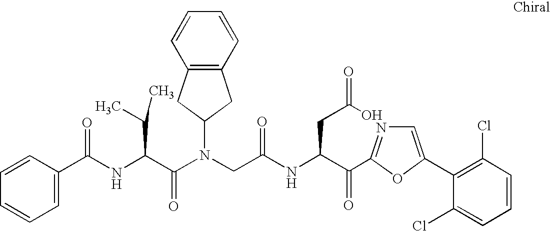 | Vertex | | WO 9722618 |
| 737 | 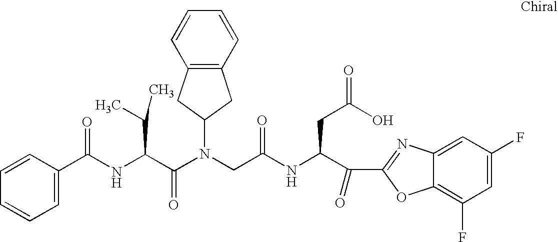 | Vertex | | WO 9722618 |
| 738 | 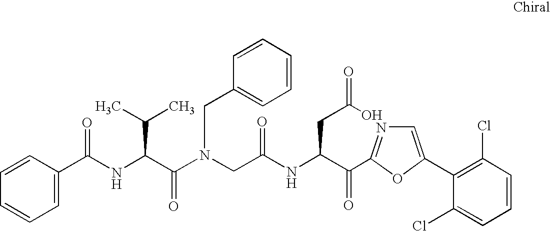 | Vertex | | WO 9722618 |
| 739 | 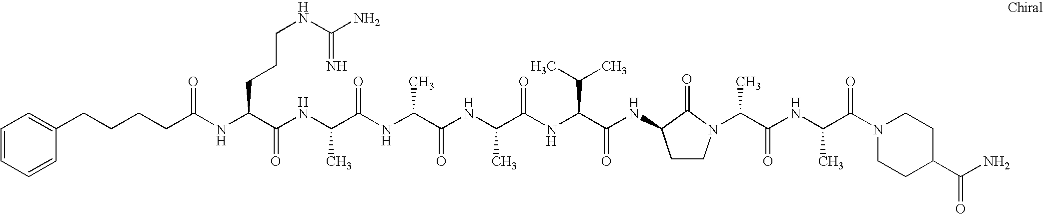 | Astra Zeneca | | WO 9731023 |
| 740 | 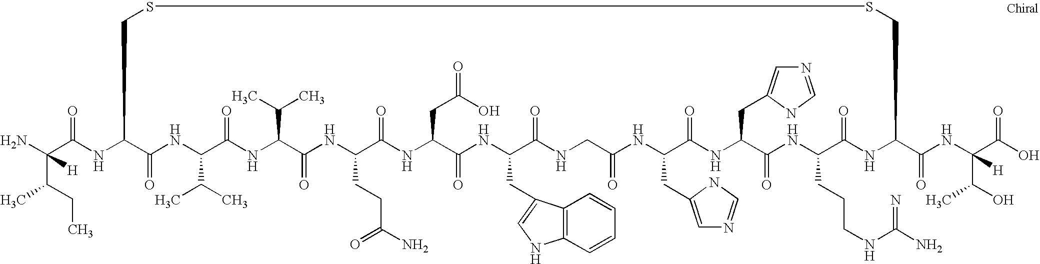 | Univer- sity of Penn- sylvania | | WO 9733603 |
| 741 | 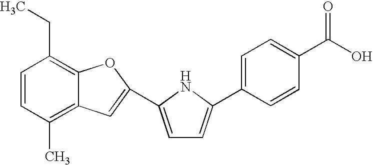 | Eisai | | EP 889032 |
| 742 | 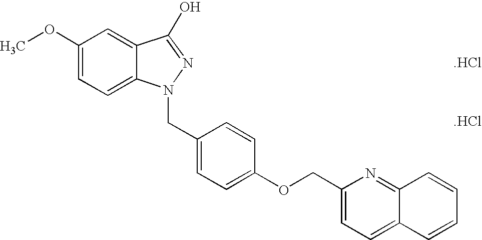 | AWD. pharma | | WO 9734874 |
| 743 | 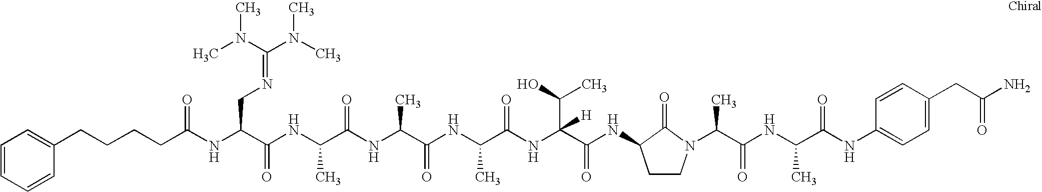 | Astra Zeneca | | WO 9731023 |
| 744 | 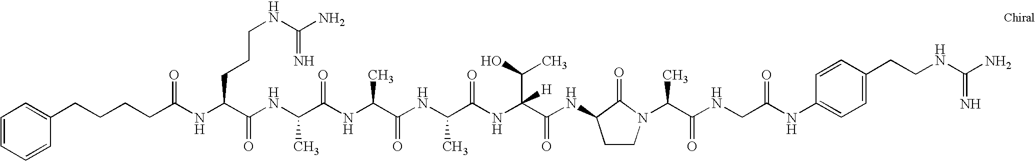 | Astra Zeneca | | WO 9731023 |
| 745 | 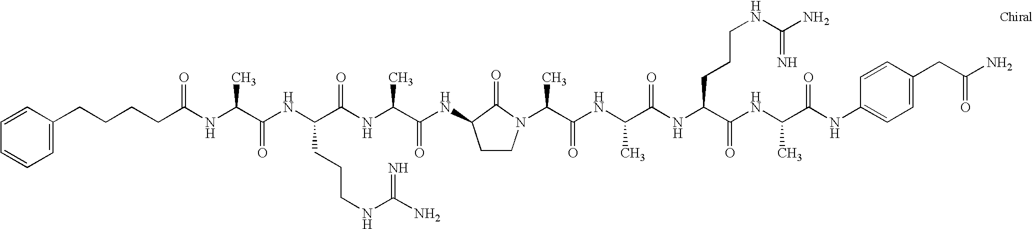 | Astra Zeneca | | WO 9731023 |
| 746 | 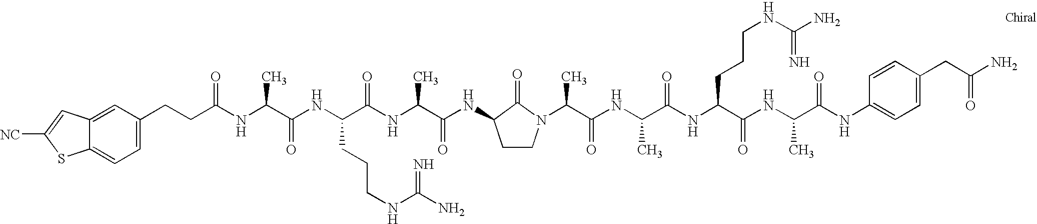 | Astra Zeneca | | WO 9731023 |
| 747 | 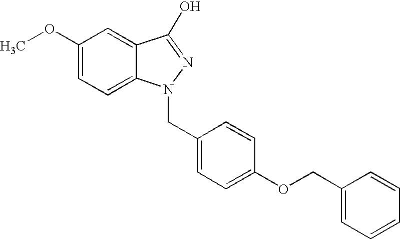 | AWD. pharma | | WO 9734874 |
| 748 | 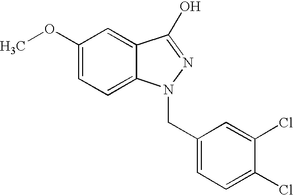 | AWD. pharma | | WO 9734874 |
| 749 | 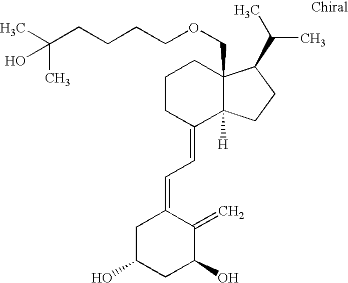 | Leo | | WO 9737972 |
| 750 | 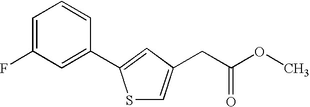 | Taisho | | JP 97194476 |
| 751 | 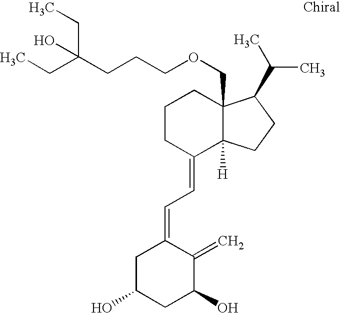 | Leo | | WO 9737972 |
| 752 | 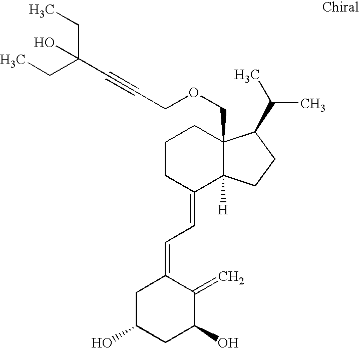 | Leo | | WO 9737972 |
| 753 | 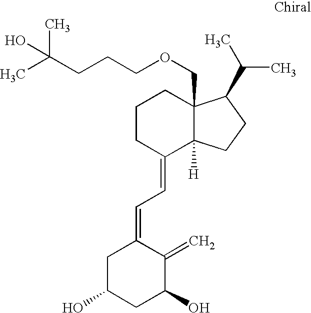 | Leo | | WO 9737972 |
| 754 | 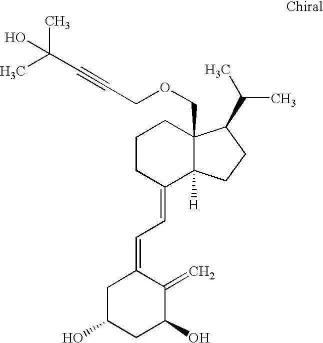 | Leo | | WO 9737972 |
| 755 | 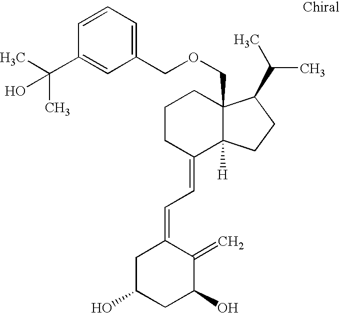 | Leo | | WO 9737972 |
| 756 | 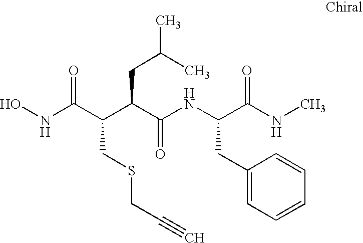 | Glaxo- Smith Kline | | WO 9743250 |
| 757 | 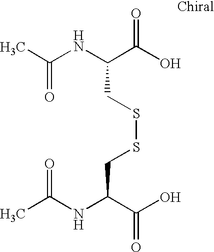 | Astra Zeneca | 1) Saem- strand, B. et al. J Pharmacol Exp Ther 1999, 228(3): 1174. | EP 0463514 |
| 758 | 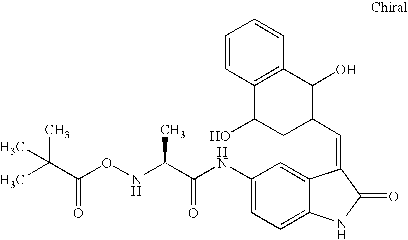 | Pharm- acia | | WO 9745409 |
| 759 | 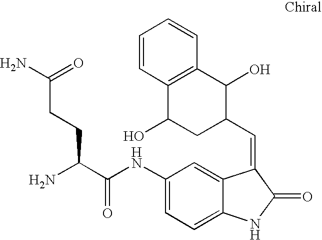 | Pharm- acia | | WO 9745409 |
| 760 | 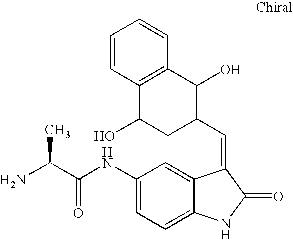 | Pharm- acia | | WO 9745409 |
| 761 | 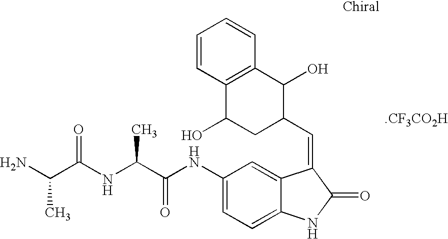 | Pharm- acia | | WO 9745409 |
| 762 | 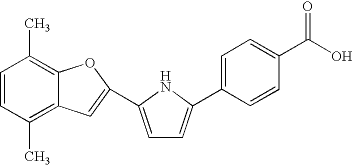 | Eisai | Nagai, M. et al. 217th ACS Natl Meet (March 21-25, Ana- heim) 1999, Abst MEDI 050. | EP 889032 |
| 763 | 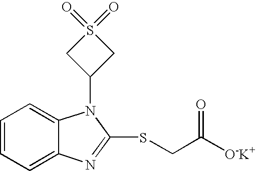 | Bashkir Medical Univer- sity | Sadykov, R. F. et al. Naunyn- Schmied Arch Pharm- acol 1998, 358(1, Suppl. 2): Abst P 52.28. | |
| 764 | 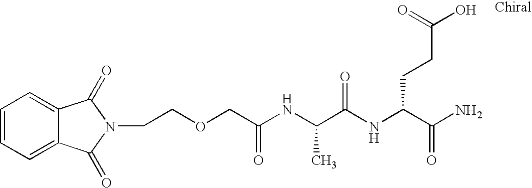 | LEK | | EP 477912 |
| 765 | 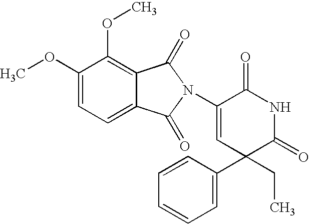 | Gruen- enthal | | EP 856513 |
| 766 | 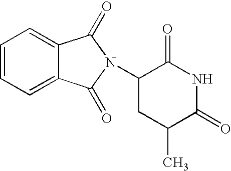 | Gruen- enthal | | EP 856513 |
| 767 | 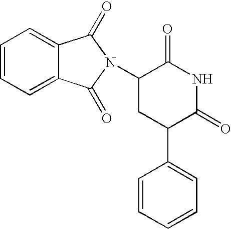 | Gruen- enthal | | EP 856513 |
| 768 | 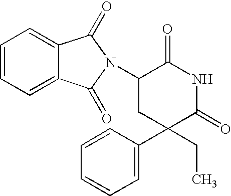 | Gruen- enthal | | EP 856513 |
| 769 | 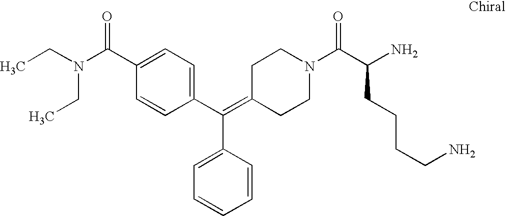 | Astra Zeneca | | WO 9828275 |
| 770 | 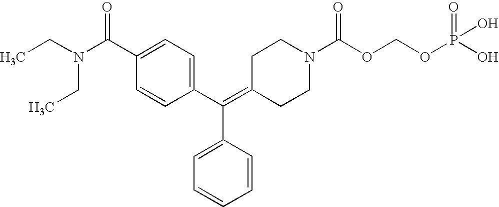 | Astra Zeneca | | WO 9828275 |
| 771 | 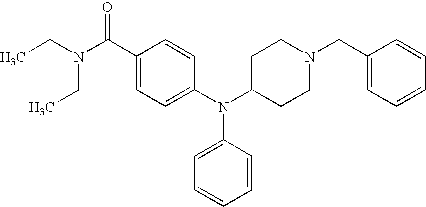 | Astra Zeneca | | WO 9828270 |
| 772 | 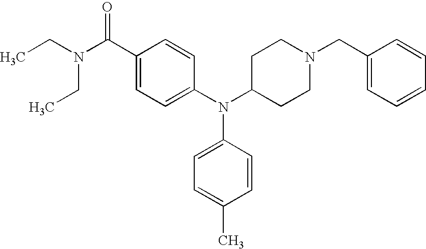 | Astra Zeneca | | WO 9828270 |
| 773 | 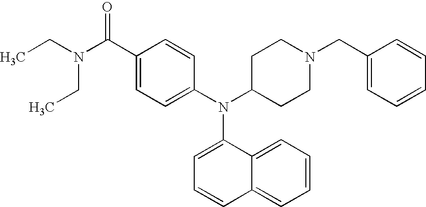 | Astra Zeneca | | WO 9828270 |
| 774 | 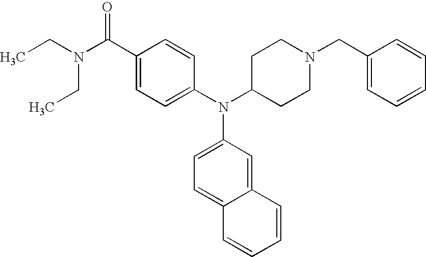 | Astra Zeneca | | WO 9828270 |
| 775 | 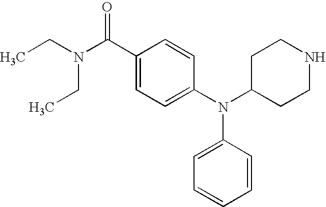 | Astra Zeneca | | WO 9828270 |
| 776 | 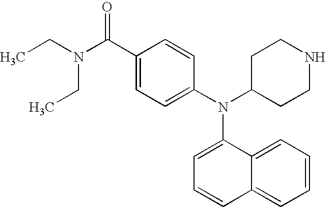 | Astra Zeneca | | WO 9828270 |
| 777 | 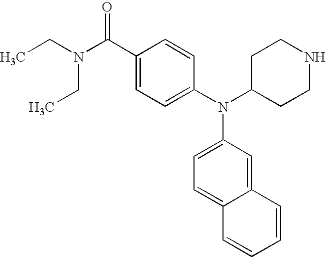 | Astra Zeneca | | WO 9828270 |
| 778 | 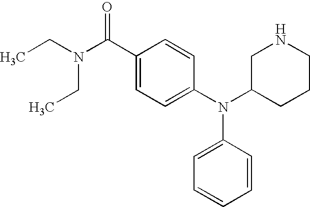 | Astra Zeneca | | WO 9828270 |
| 779 | 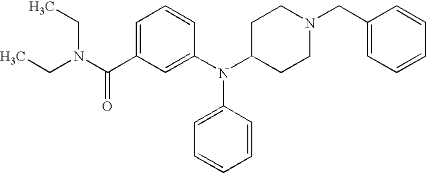 | Astra Zeneca | | WO 9828270 |
| 780 | 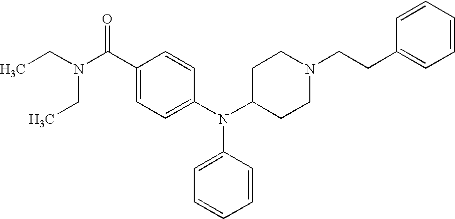 | Astra Zeneca | | WO 9828270 |
| 781 | 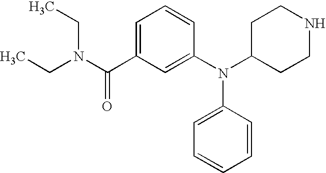 | Astra Zeneca | | WO 9828270 |
| 782 | 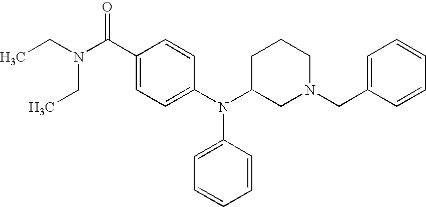 | Astra Zeneca | | WO 9828270 |
| 783 | 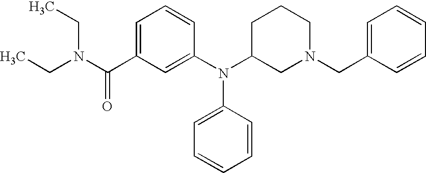 | Astra Zeneca | | WO 9828270 |
| 784 | 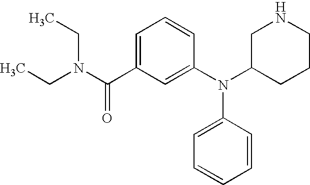 | Astra Zeneca | | WO 9828270 |
| 785 | 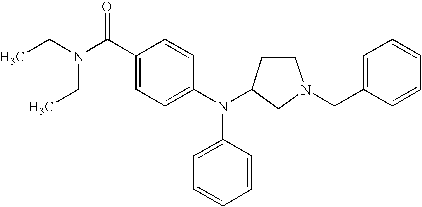 | Astra Zeneca | | WO 9828270 |
| 786 | 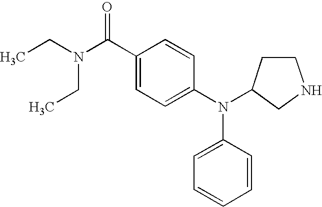 | Astra Zeneca | | WO 9828270 |
| 787 | 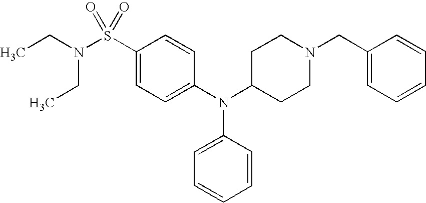 | Astra Zeneca | | WO 9828270 |
| 788 | 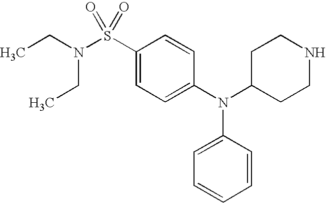 | Astra Zeneca | | WO 9828270 |
| 789 | 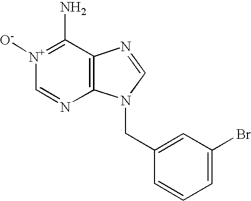 | Japan Energy | | JP 98231297 |
| 790 | 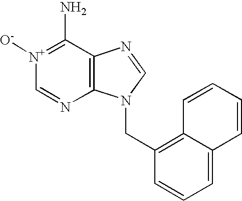 | Japan Energy | | JP 98231297 |
| 791 | 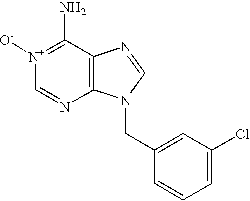 | Japan Energy | | JP 98231297 |
| 792 | 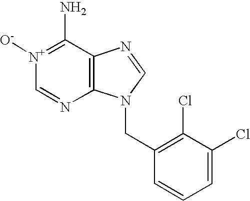 | Japan Energy | | JP 98231297 |
| 793 | 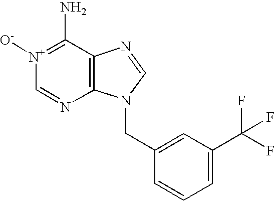 | Japan Energy | | JP 98231297 |
| 794 | 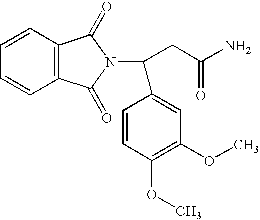 | Celgene | 1) Moreira, A. L. et al. J Neuro- Oncol 1999, 43(2): 109. | U.S. Pat. No. 5463063 |
| 795 | 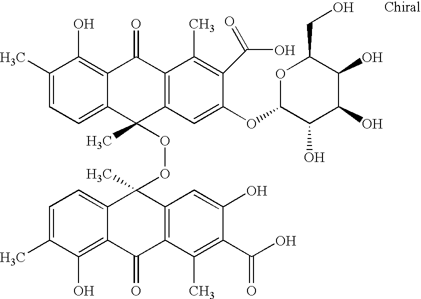 | Daiichi Pharma- ceutical | Koiwa, T. et al. J Antibiot 1999, 52(2): 198. | |
| 796 | 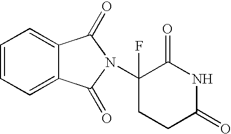 | Celgene | | U.S. Pat. No. 5874448 |
| 797 | 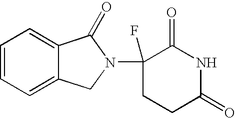 | Celgene | | U.S. Pat. No. 5874448 |
| 798 | 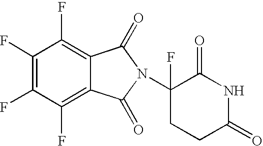 | Celgene | | U.S. Pat. No. 5874448 |
| 799 | 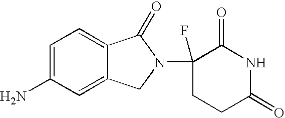 | Celgene | | U.S. Pat. No. 5874448 |
| 800 | 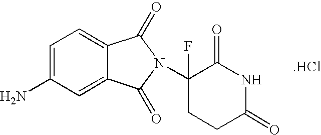 | Celgene | | U.S. Pat. No. 5874448 |
| 801 | 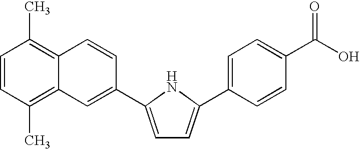 | Eisai | | EP 889032 |
| 802 | 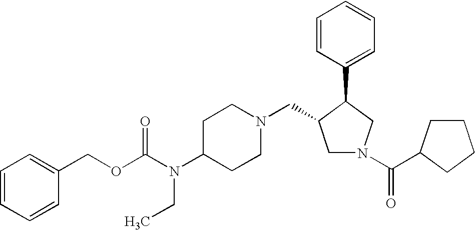 | Merck & Co. | | WO 9909984 |
| 803 | 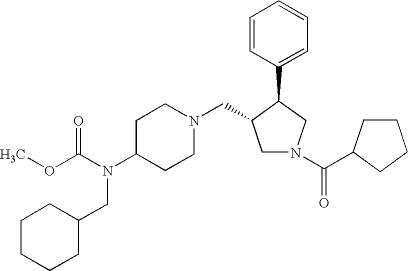 | Merck & Co. | | WO 9909984 |
| 804 | 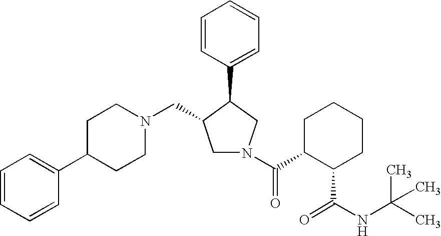 | Merck & Co. | | WO 9909984 |
| 805 | 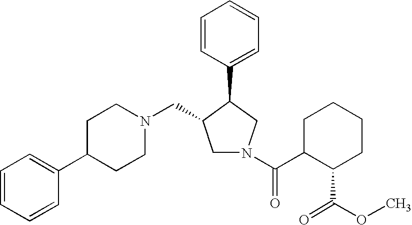 | Merck & Co. | | WO 9909984 |
| 806 | 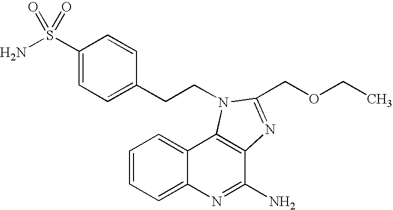 | Hokur- iku | | JP 99080156 |
| 807 | 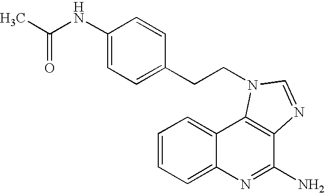 | Hokur- iku | | JP 99080156 |
| 808 | 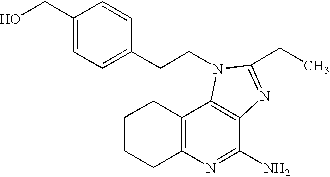 | Hokur- iku | | JP 99080156 |
| 809 | 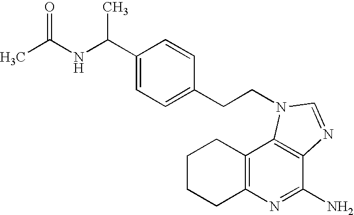 | Hokur- iku | | JP 99080156 |
| 810 | 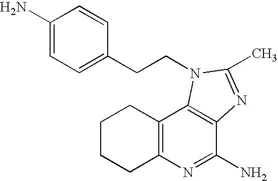 | Hokur- iku | | JP 99080156 |
| 811 | 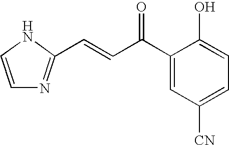 | SSP | | CA 2255337 |
| 812 | 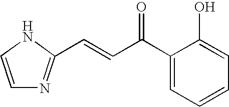 | SSP | | CA 2255337 |
| 813 | 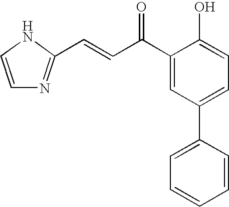 | SSP | | CA 2255337 |
| 814 | 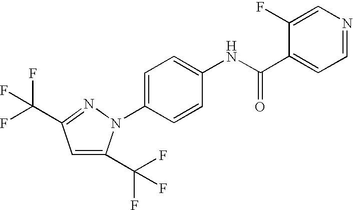 | Abbott | Madar, D. et al. 222nd ACS Natl Meet (Aug 26-30, Chicago) 2001, Abst MEDI 7. | EP 1068187 |
| 815 | 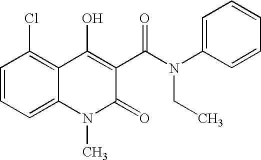 | Active Biotech | | WO 9955678 |
| 816 | 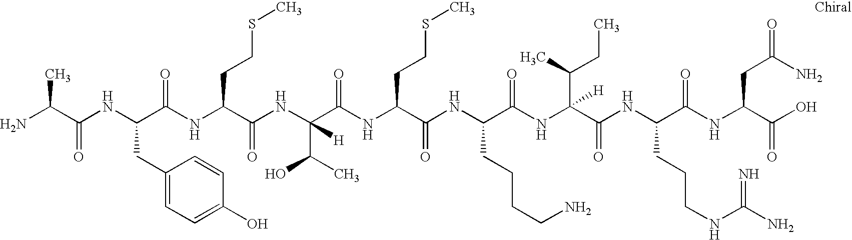 | Amer- sham | 1) Gesser, B. et al. Proc Natl Acad Sci USA 1997, 94(26): 14620. | WO 9601318 |
| 817 | 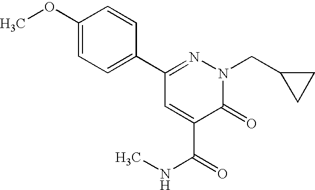 | Kowa | | WO 9944995 |
| 818 | 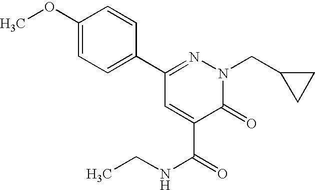 | Kowa | | WO 9944995 |
| 819 | 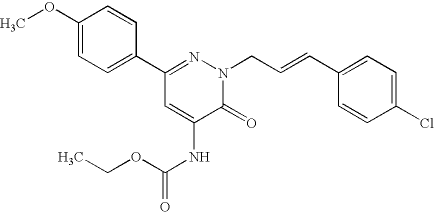 | Kowa | | WO 9944995 |
| 820 | 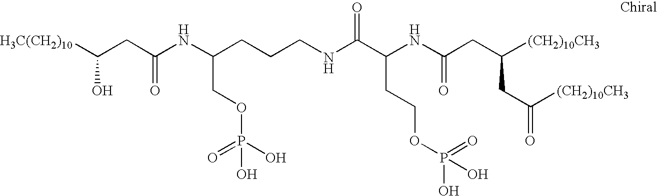 | OM Pharma | | WO 0000462 |
| 821 | 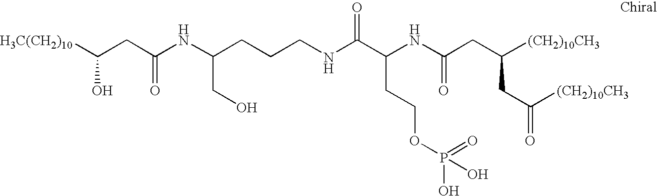 | OM Pharma | | WO 0000462 |
| 822 | 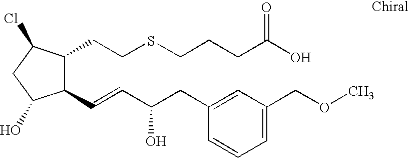 | Ono | | WO 0003980 |
| 823 | 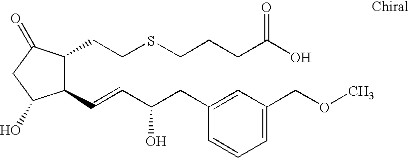 | Ono | | WO 0003980 |
| 824 | 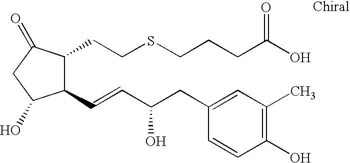 | Ono | | WO 0003980 |
| 825 | 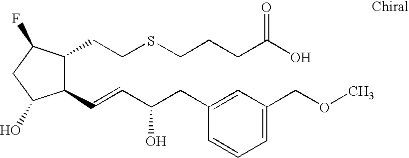 | Ono | | WO 0003980 |
| 826 | 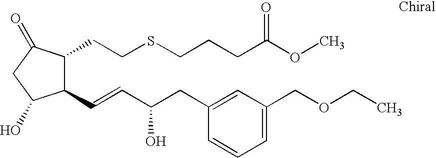 | Ono | | WO 0003980 |
| 827 | 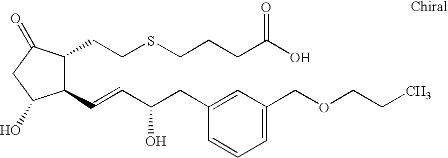 | Ono | | WO 0003980 |
| 828 | 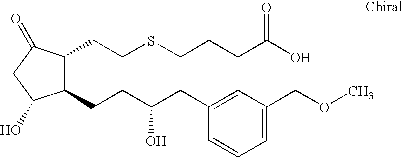 | Ono | | WO 0003980 |
| 829 | 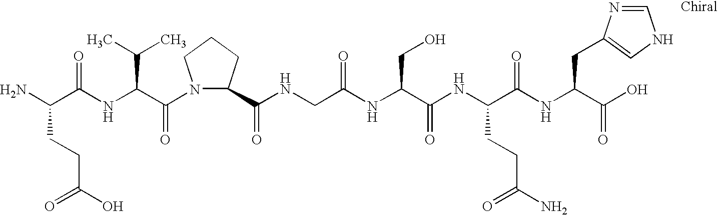 | Univer- sity of Bristol | | WO 0014114 |
| 830 | 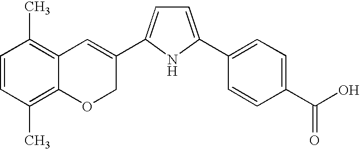 | Eisai | Hibi, S. et al. Bioorg Med Chem Lett 2000, 10(7): 623. | EP 0889032 |
| 831 | 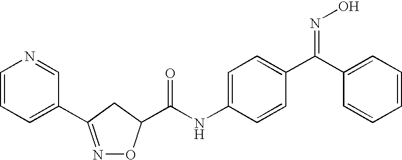 | Janssen | | WO 0021959 |
| 832 | 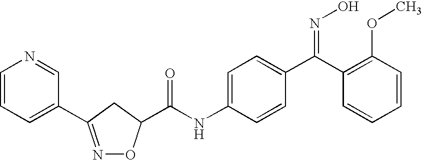 | Janssen | | WO 0021959 |
| 833 | 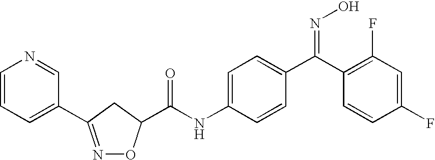 | Janssen | | WO 0021959 |
| 834 | 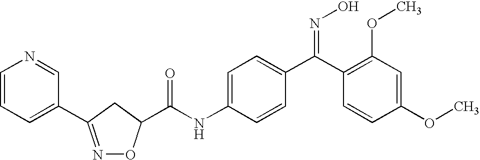 | Janssen | | WO 0021959 |
| 835 | 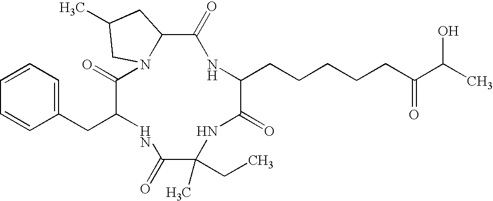 | Fuji- sawa | | WO 0021979 |
| 836 | 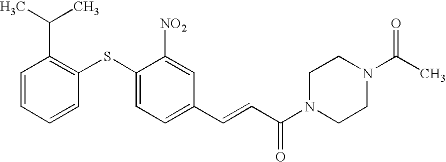 | Abbott | 1) Liu, G. et al. 220th ACS Natl Meet (Aug 20-24, Washington DC) 2000, Abst MEDI 171. | 1) U.S. Pat. No. 6110922 |
| 837 | 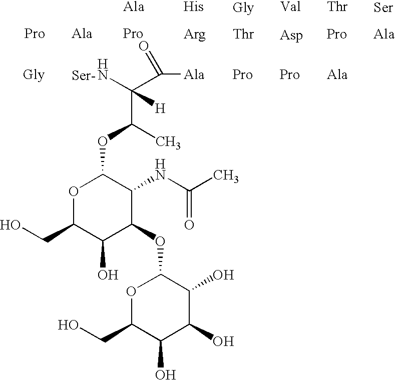 | Cancer Re- search UK | | WO 0052046 |
| 838 | 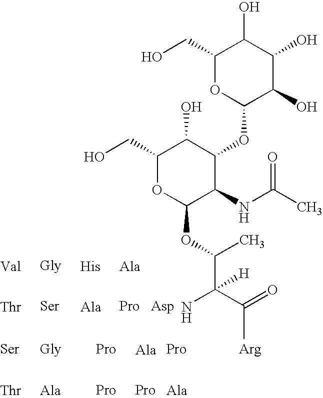 | Cancer Re- search UK | | WO 0052046 |
| 839 | 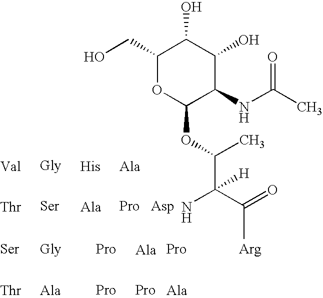 | Cancer Re- search UK | | WO 0052046 |
| 840 | 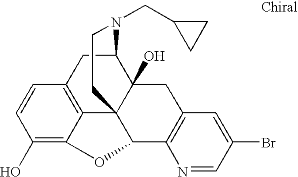 | South- ern Re- search Institute | | WO 0112197 |
| 841 | 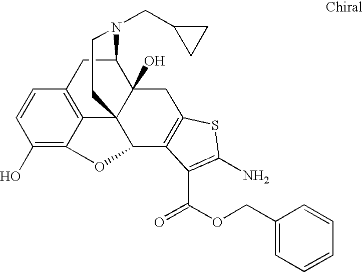 | South- ern Re- search Institute | | WO 0112197 |
| 842 | 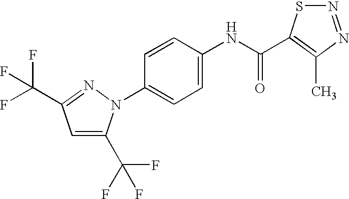 | Abbott Labs. | | EP 1068187 |
| 843 | 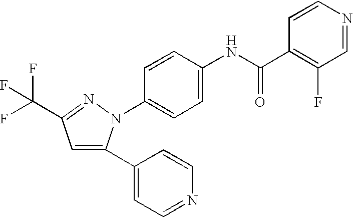 | Abbott | | EP 1068187 |
| 844 | 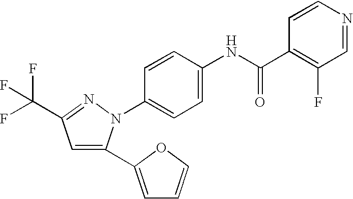 | Abbott | | EP 1068187 |
| 845 | 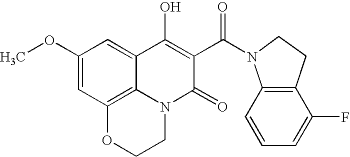 | Fuji- sawa | Spears, G. et al. 21st Symp Med Chem (Nov 28-30, Kyoto) 2001, Abst 2P-29. | |
| 846 | 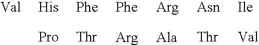 | Austin Re- search Institute | Tselios, T. et al. J Med Chem 2002, 45(2): 275. | |
| 847 | 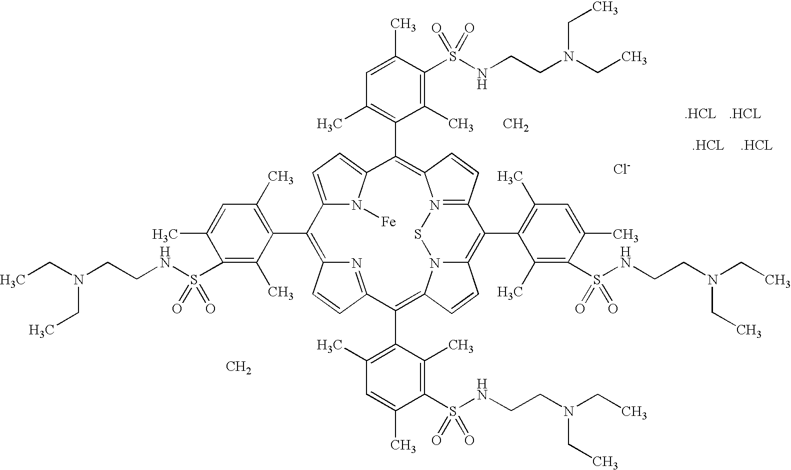 | Eukar- ion | | WO 0204454 |
| 848 | 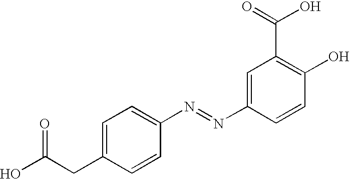 | Nobex | 1) Riggs- Sauthier, J. A. et al. 224th ACS Natl Meet (Aug 18-22, Boston) 2002, Abst MEDI 305. | WO 0218324 |
| 849 | 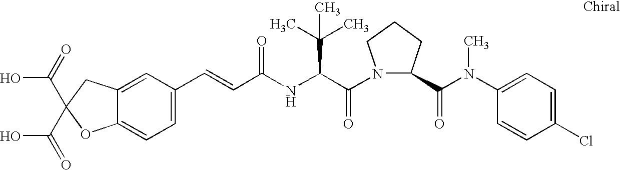 | Tularik | | WO 0238107 |
| 850 | 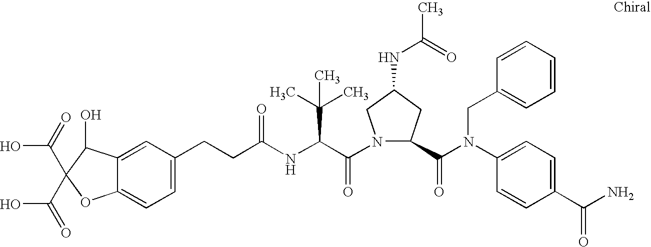 | Tularik | | WO 0238107 |
| 851 | 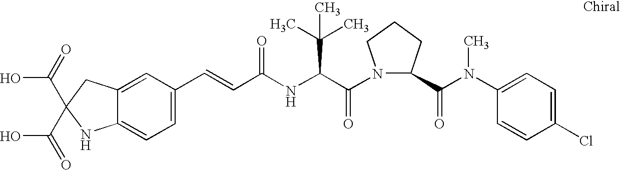 | Tularik | | WO 0238107 |
| 852 | 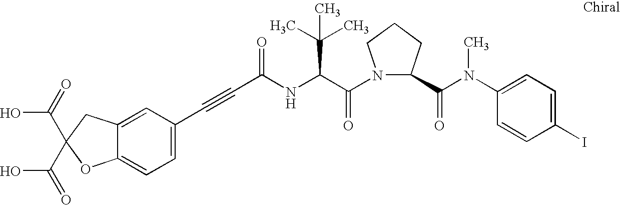 | Tularik | | WO 0238107 |
| 853 | 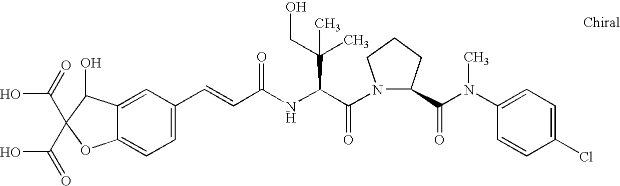 | Tularik | | WO 0238107 |
| 854 | 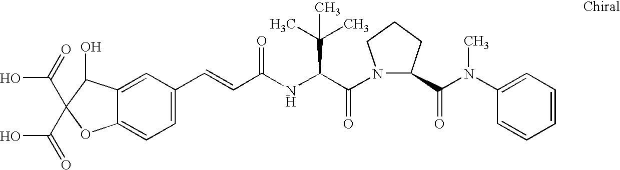 | Tularik | | WO 0238107 |
| 855 | 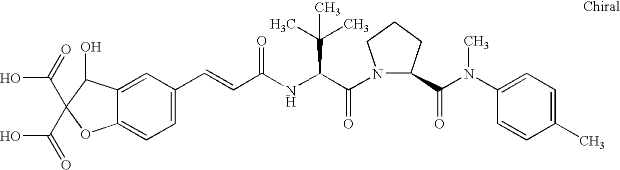 | Tularik | | WO 0238107 |
| 856 | 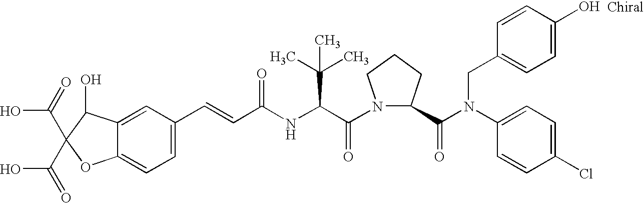 | Tularik | | WO 0238107 |
| 857 | 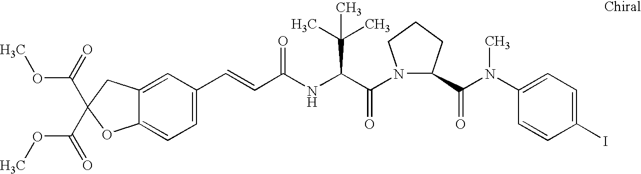 | Tularik | | WO 0238107 |
| 858 | 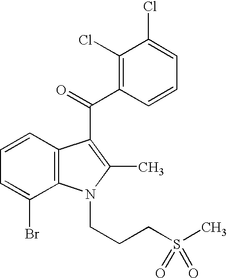 | Sanofi- Synthe labo | | WO 0242269 |
| 859 | 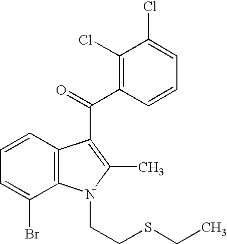 | Sanofi- Synthe labo | | WO 0242269 |
| 860 | 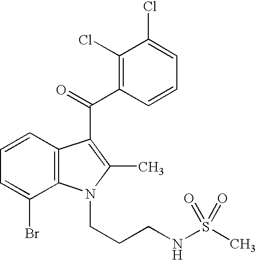 | Sanofi- Synthe labo | | WO 0242269 |
| 861 | 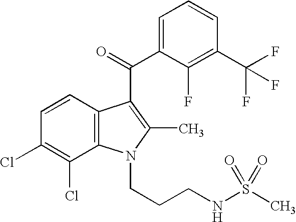 | Sanofi- Synthe labo | | WO 0242269 |
| 862 | 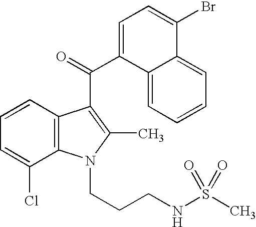 | Sanofi- Synthe labo | | WO 0242269 |
| 863 | 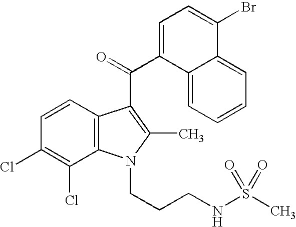 | Sanofi- Synthe labo | | WO 0242269 |
| 864 | 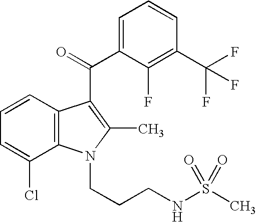 | Sanofi- Synthe labo | | WO 0242269 |
| 865 | 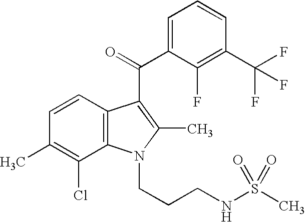 | Sanofi- Synthe labo | | WO 0242269 |
| 866 | 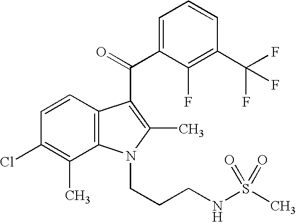 | Sanofi- Synthe labo | | WO 0242269 |
| 867 | 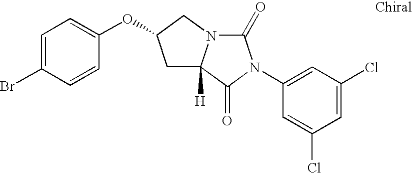 | Bristol- Myers Squibb | | WO 0244181 |
| 868 | 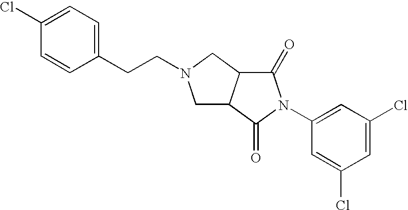 | Bristol- Myers Squibb | | WO 0244181 |
| 869 | 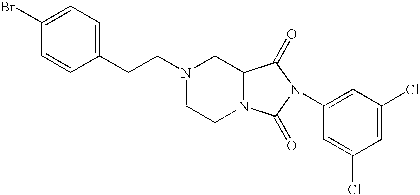 | Bristol- Myers Squibb | | WO 0244181 |
| 870 | 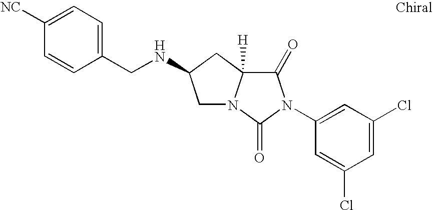 | Bristol- Myers Squibb | | WO 0244181 |
| 871 | 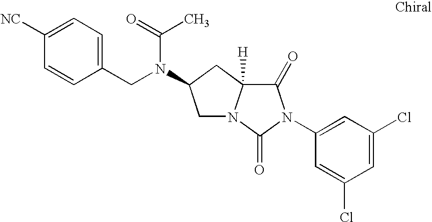 | Bristol- Myers Squibb | | WO 0244181 |
| 872 | 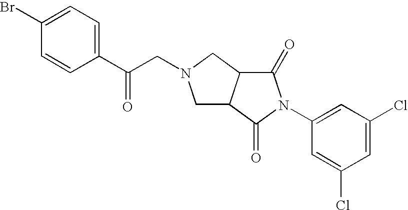 | Bristol- Myers Squibb | | WO 0244181 |
| 873 | 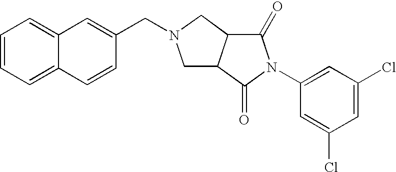 | Bristol- Myers Squibb | | WO 0244181 |
| 874 | 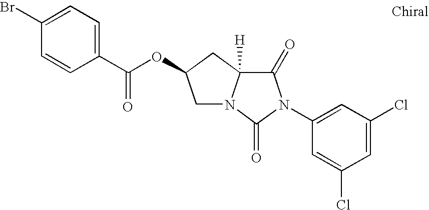 | Bristol- Myers Squibb | | WO 0244181 |
| 875 | 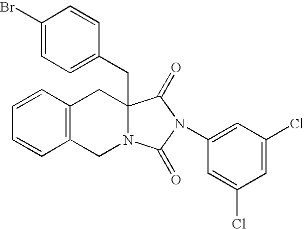 | Bristol- Myers Squibb | | WO 0244181 |
| 876 | 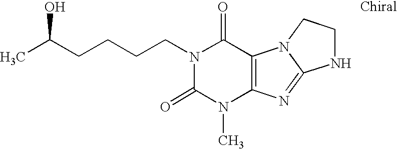 | Cell Thera- peutics | | WO 0268421 |
| 877 | 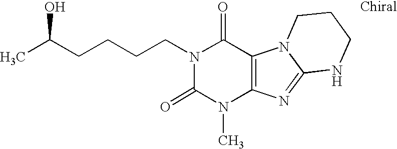 | Cell Thera- peutics | | WO 0268421 |
| 878 | 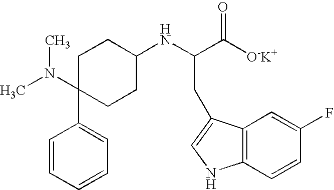 | Gruen- enthal | | WO 0290317 |
| 881 | 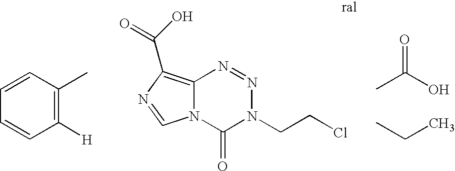 | May & Baker | | EP 252682 |
| 882 | 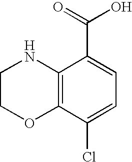 | Kyorin | | EP 310096 |
| 883 | 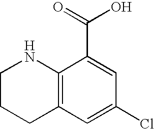 | Kyorin | | EP 310096 |
| 884 | 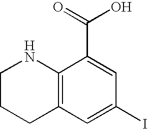 | Kyorin | | EP 310096 |
| 885 | 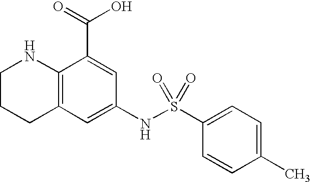 | Kyorin | | EP 310096 |
| 886 | 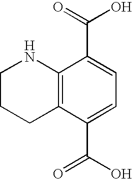 | Kyorin | | EP 310096 |
| 887 | 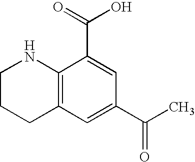 | Kyorin | | EP 310096 |
| 888 | 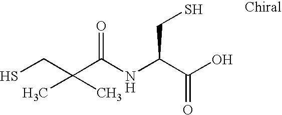 | Santen | | EP 326326 |
| 889 | 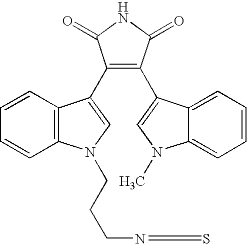 | Roche | | AU 8929658 |
| 890 | 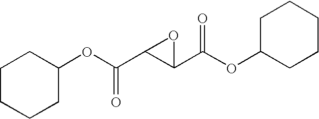 | Taisho | 1) Takeshita, K. et al. Int J Immunother 1988, 4(2): 97-106. | JP 79141750 |
| 891 | 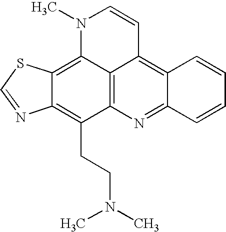 | Harbor Branch Found. | 1) Burres, N. S. et al. Proc Amer Assoc Cancer Res 1989, 30: Abst 1914. | EP 331320 |
| 892 | 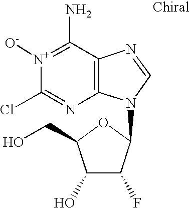 | Scripps Clinic Res. Found. | | WO 8908658 |
| 893 | 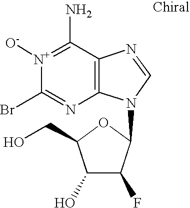 | Scripps Clinic Res. Found. | | WO 8908658 |
| 894 | 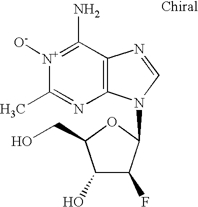 | Scripps Clinic Res. Found. | | WO 8908658 |
| 895 | 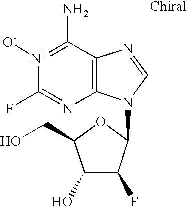 | Scripps Clinic Res. Found. | | WO 8908658 |
| 896 | 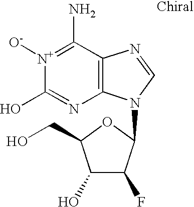 | Scripps Clinic Res. Found. | | WO 8908658 |
| 897 | 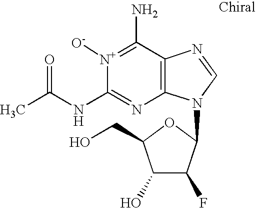 | Scripps Clinic Res. Found. | | WO 8908658 |
| 898 | 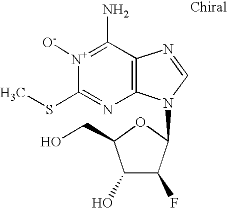 | Scripps Clinic Res. Found. | | WO 8908658 |
| 899 | 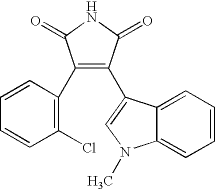 | Roche | | AU 8929658 |
| 900 | 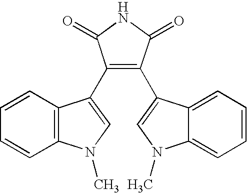 | Roche | | AU 8929658 |
| 901 | 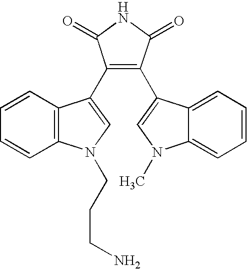 | Roche | | AU 8929658 |
| 902 | 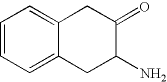 | Aventis Pharma | | EP 378456 |
| 903 | 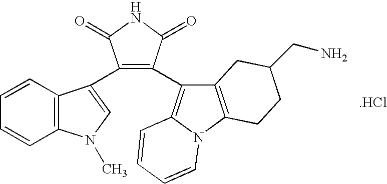 | Roche | | EP 384349 |
| 904 | 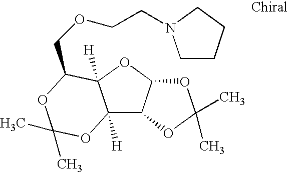 | Green- wich Pharm. | | AU 9047648 |
| 905 | 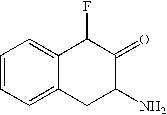 | Aventis Pharma | | EP 378456 |
| 906 | 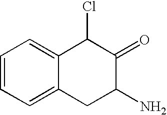 | Aventis Pharma | | EP 378456 |
| 907 | 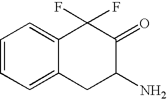 | Aventis Pharma | | EP 378456 |
| 908 | 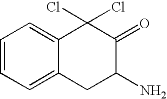 | Aventis Pharma | | EP 378456 |
| 909 | 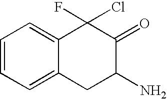 | Aventis Pharma | | EP 378456 |
| 910 | 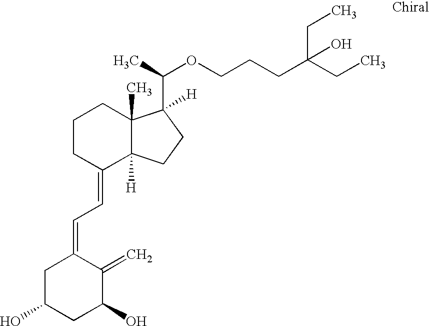 | Leo | Binderup, L. et al. Biochem Pharmacol 1991, 42(8): 1569. | EP 460032 |
| 911 | 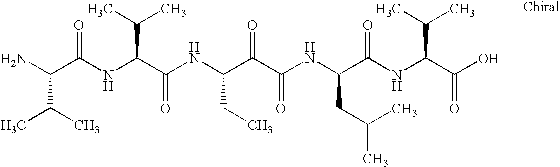 | Micro- bial Chem- istry Re- search Founda- tion | Muraoka, Y. et al. 30th Intersci Conf Antimicrob Agents Chemother (Oct 21-24, Atlanta) 1990, Abst 801. | |
| 912 | 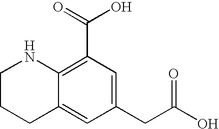 | Kyorin | | AU 9057029 |
| 913 | 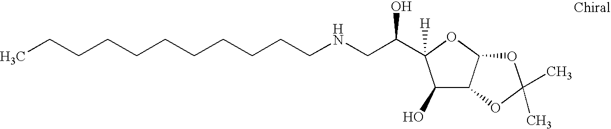 | Green- wich Pharm. | | AU 9057691 |
| 914 | 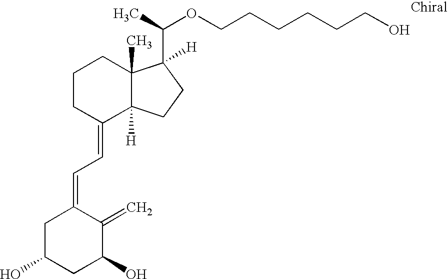 | Leo | | EP 460032 |
| 915 | 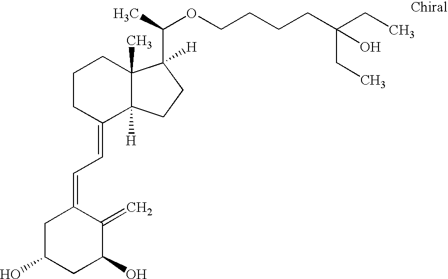 | Leo | | EP 460032 |
| 916 | 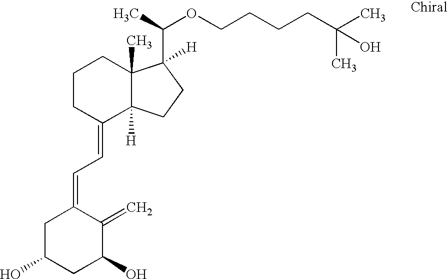 | Leo | | EP 460032 |
| 917 | 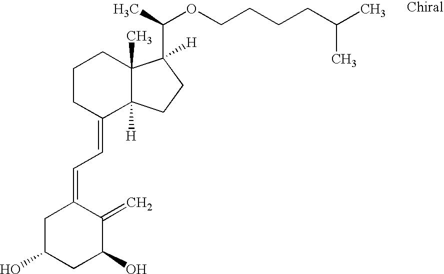 | Leo | | EP 460032 |
| 918 | 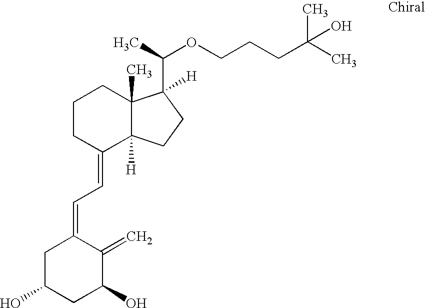 | Leo | | EP 460032 |
| 919 | 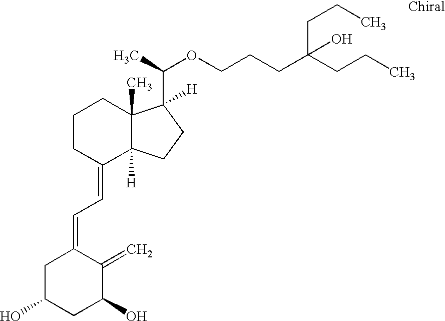 | Leo | | EP 460032 |
| 920 | 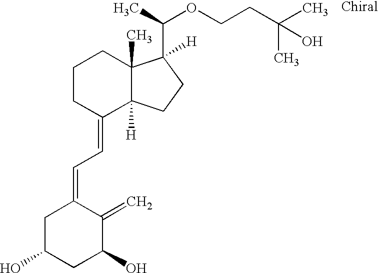 | Leo | | EP 460032 |
| 921 | 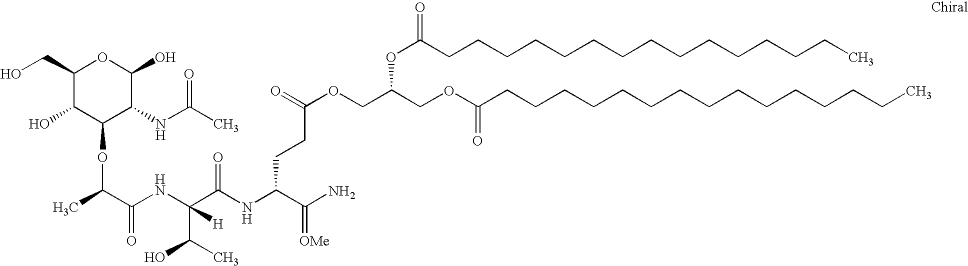 | Novar- tis | Kricek, F. et al. Immuno- pharmacol- ogy 1997, 36(1): 27. | AU 9057875 |
| 922 | 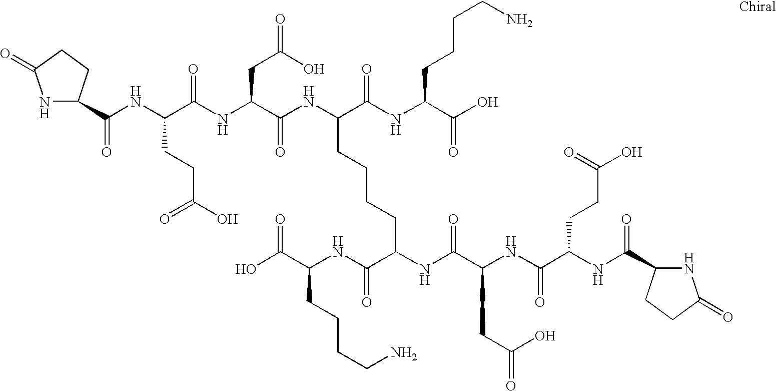 | Amer- sham Health | 1) Frey, C. L. et al. 31st Intersci Conf Anti- microb Agents Chemother (Sept 29-Oct 2, Chicago) 1991, Abst 85. | AU 9059014 |
| 923 | 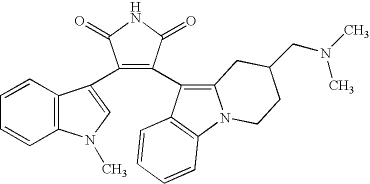 | Roche | | EP 384349 |
| 924 | 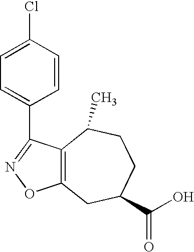 | Roche | | AU 9059154 |
| 925 | 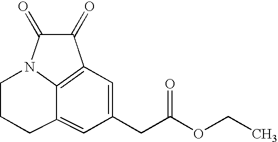 | Kyorin | | AU 9057029 |
| 926 | 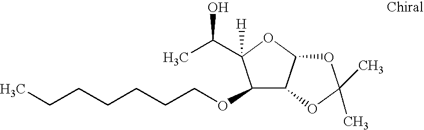 | Green- wich Pharm. | | AU 9057691 |
| 927 | 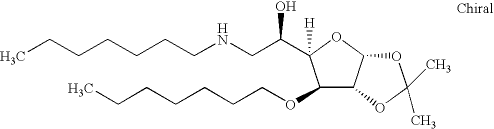 | Green- wich Pharm. | | AU 9057691 |
| 928 | 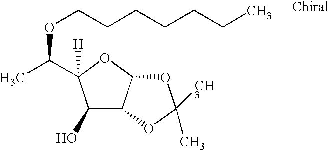 | Green- wich Pharm. | | AU 9057691 |
| 929 | 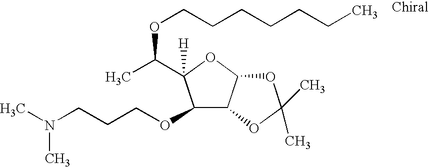 | Green- wich Pharm. | | AU 9057691 |
| 930 | 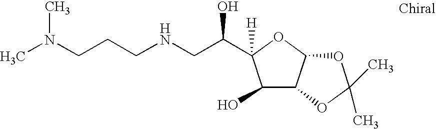 | Green- wich Pharm. | | AU 9057691 |
| 931 | 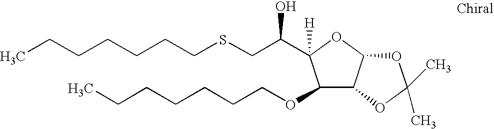 | Green- wich Pharm. | | AU 9057691 |
| 932 | 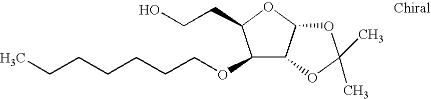 | Green- wich Pharm. | | AU 9057691 |
| 933 | 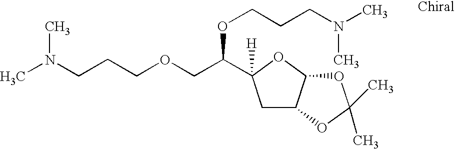 | Green- wich Pharm. | | AU 9057691 |
| 934 | 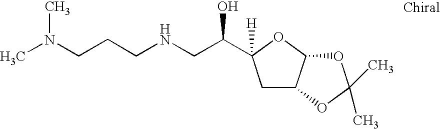 | Green- wich Pharm. | | AU 9057691 |
| 935 | 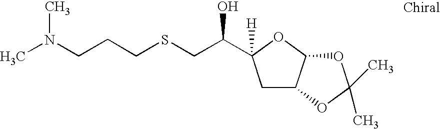 | Green- wich Pharm. | | AU 9057691 |
| 936 | 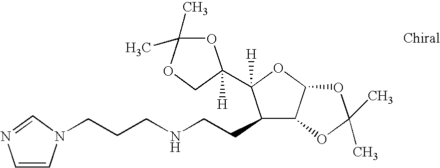 | Green- wich Pharm. | | AU 9057691 |
| 937 | 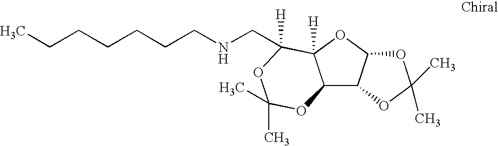 | Green- wich Pharm. | | AU 9057691 |
| 938 | 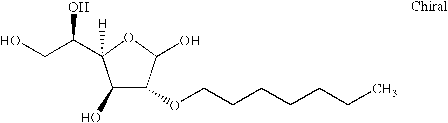 | Green- wich Pharm. | | AU 9057691 |
| 939 | 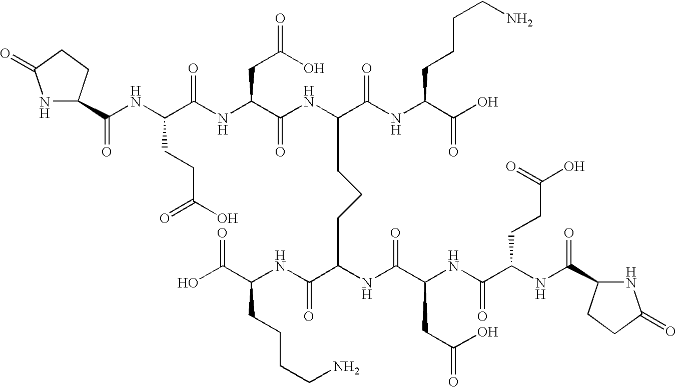 | Amer- sham Health | | AU 9059014 |
| 940 | 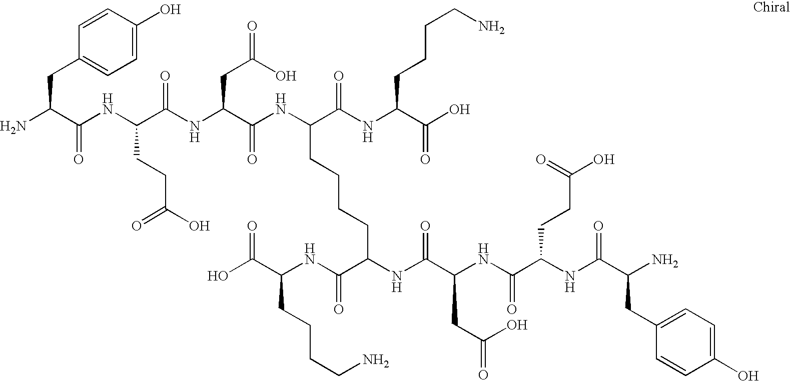 | Amer- sham Health | | AU 9059014 |
| 941 | 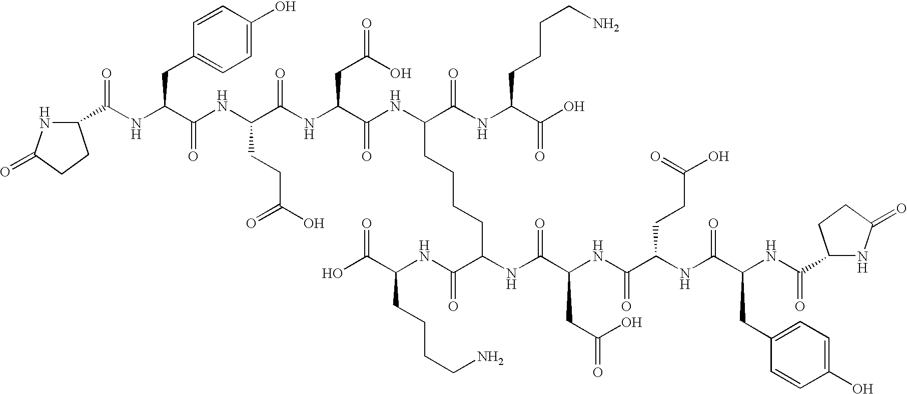 | Amer- sham Health | | AU 9059014 |
| 942 | 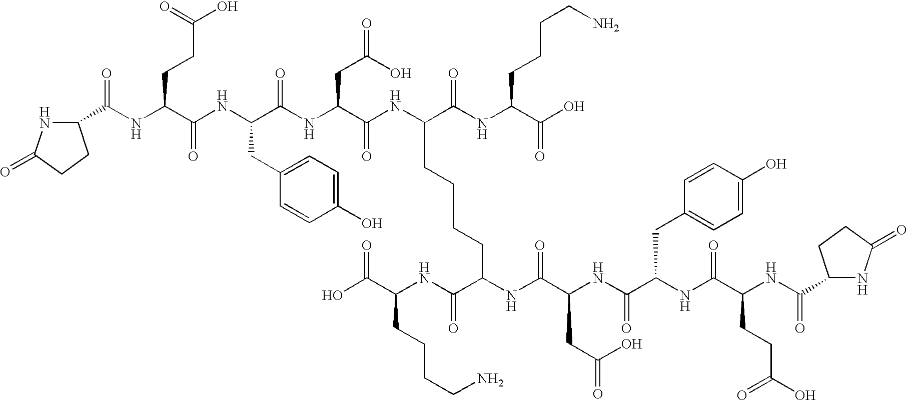 | Amer- sham Health | | AU 9059014 |
| 943 | 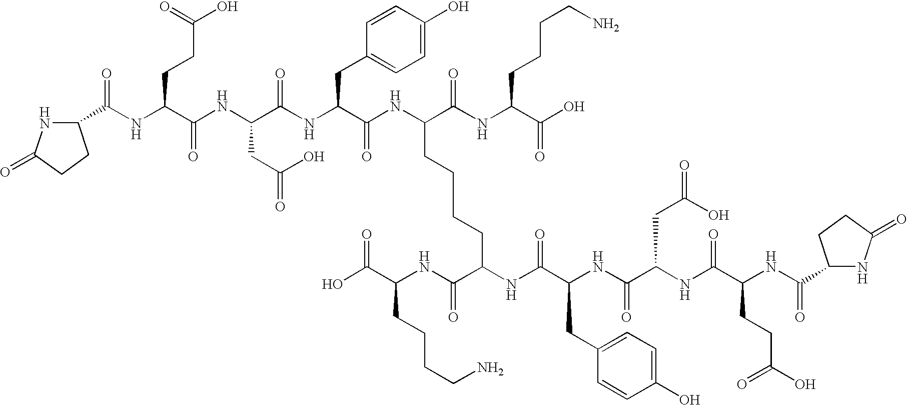 | Amer- sham Health | | AU 9059014 |
| 944 | 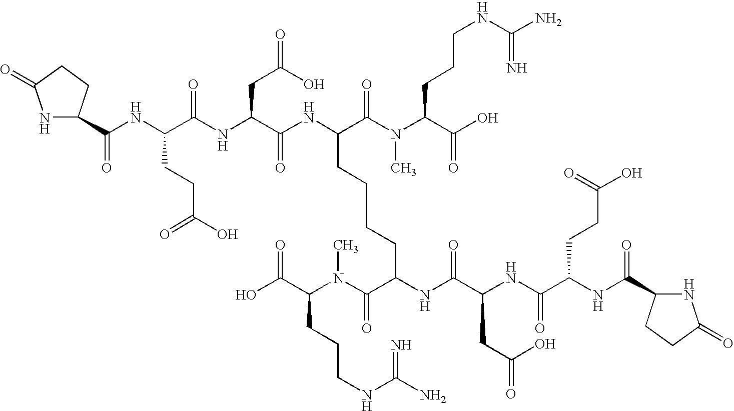 | Amer- sham Health | | AU 9059014 |
| 945 | 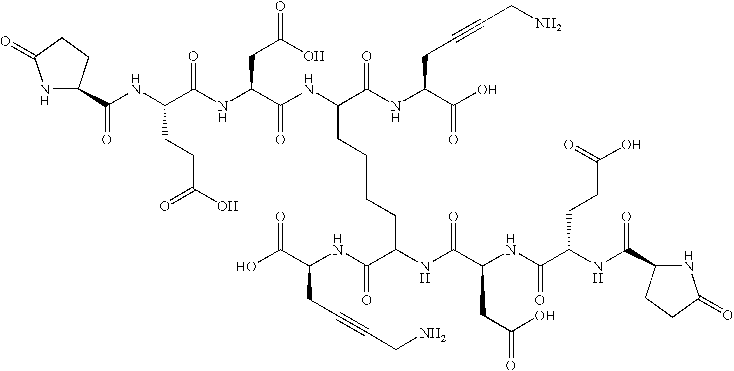 | Amer- sham Health | | AU 9059014 |
| 946 | 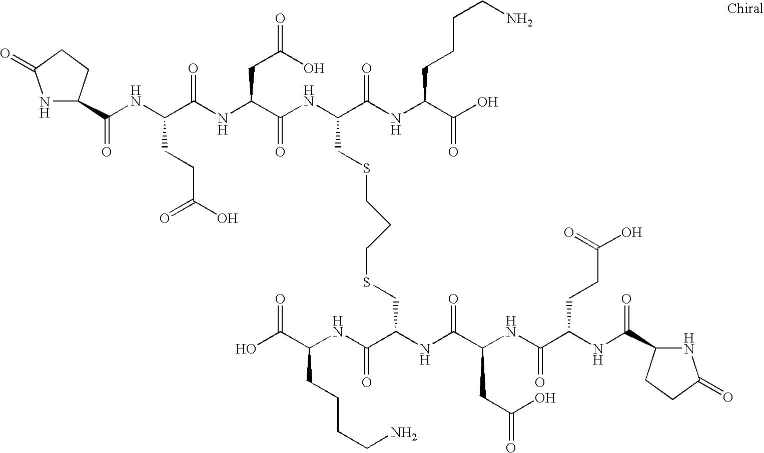 | Amer- sham Health | | AU 9059014 |
| 947 | 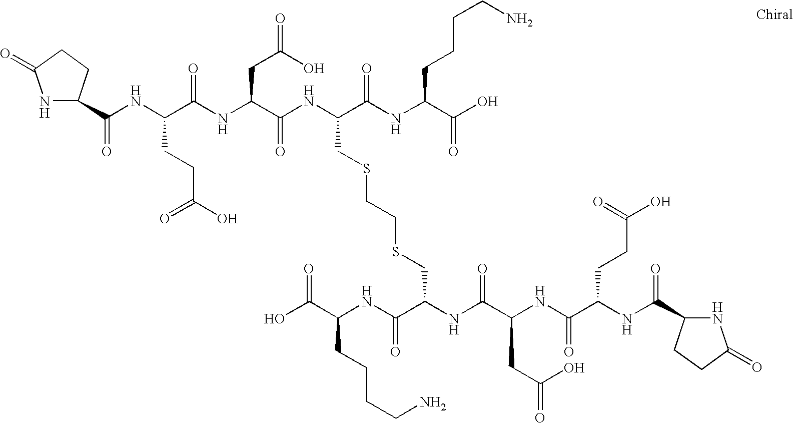 | Amer- sham Health | | AU 9059014 |
| 948 | 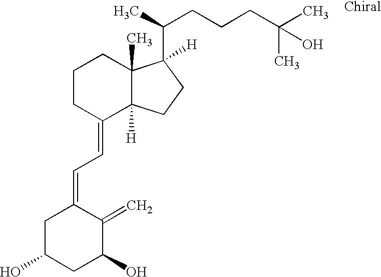 | Leo | 1) Elstner, E. et al. Blood 1994, 84(6): 1960. | 1) EP 479871 |
| 949 | 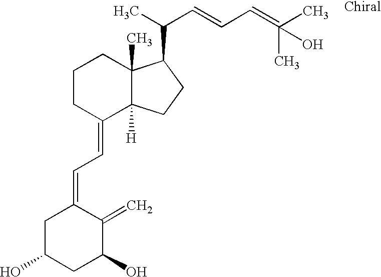 | Leo | | WO 9100855 |
| 950 | 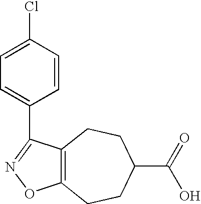 | Roche | | AU 9059154 |
| 951 | 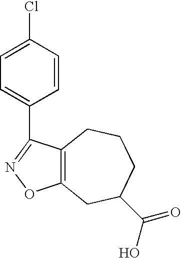 | Roche | | AU 9059154 |
| 952 | 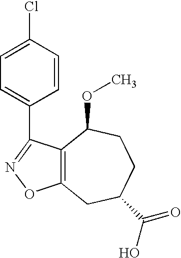 | Roche | | AU 9059154 |
| 953 | 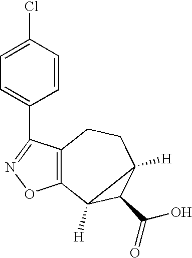 | Roche | | AU 9059154 |
| 954 | 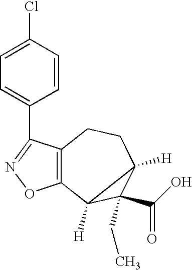 | Roche | | AU 9059154 |
| 955 | 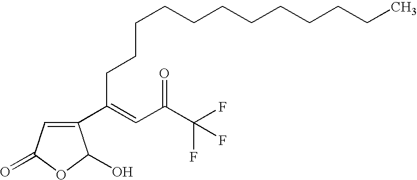 | Aller- gan | | U.S. Pat. No. 5013850 |
| 956 | 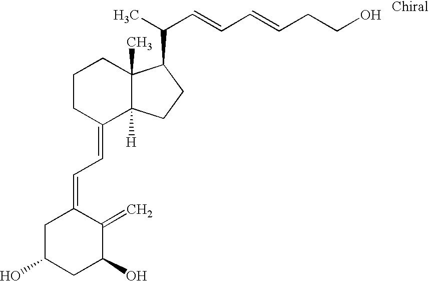 | Leo | | WO 9100855 |
| 957 | 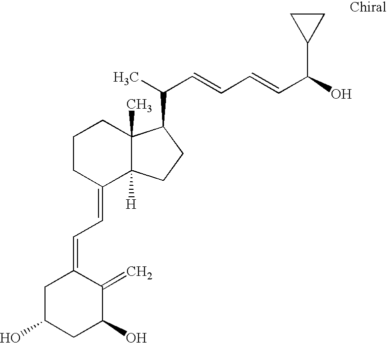 | Leo | | WO 9100855 |
| 958 | 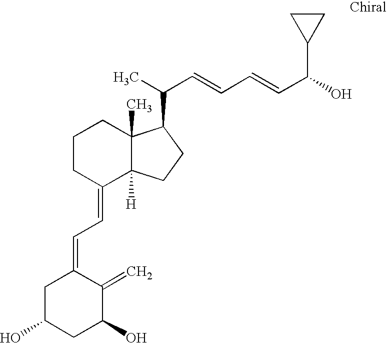 | Leo | | WO 9100855 |
| 959 | 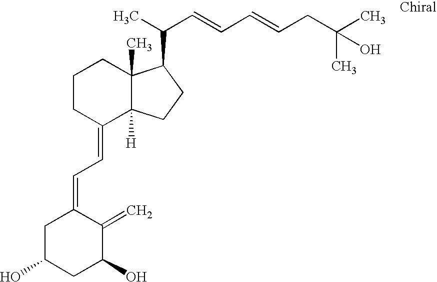 | Leo | | WO 9100855 |
| 960 | 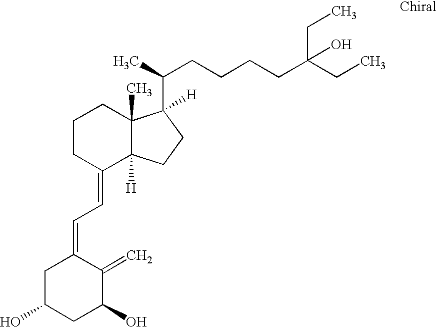 | Leo | | EP 479871 |
| 961 | 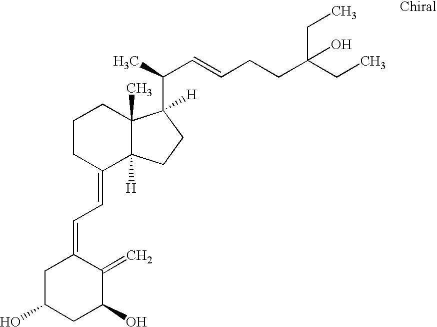 | Leo | | EP 479871 |
| 962 | 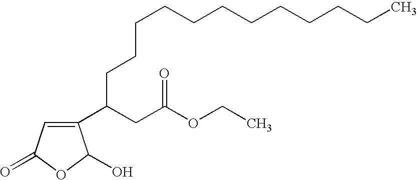 | Aller- gan | | U.S. Pat. No. 5013850 |
| 963 | 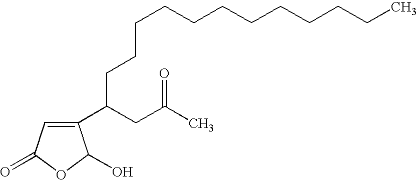 | Aller- gan | | U.S. Pat. No. 5013850 |
| 964 | 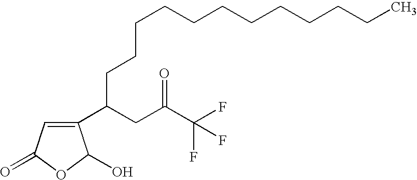 | Aller- gan | | U.S. Pat. No. 5013850 |
| 965 | 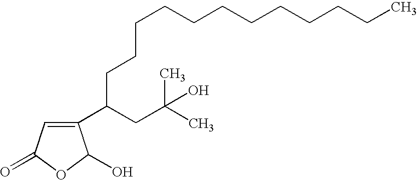 | Aller- gan | | U.S. Pat. No. 5013850 |
| 966 | 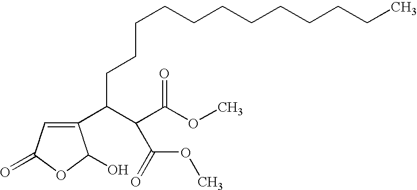 | Aller- gan | | U.S. Pat. No. 5013850 |
| 967 | 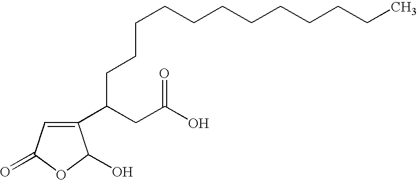 | Aller- gan | | U.S. Pat. No. 5013850 |
| 968 | 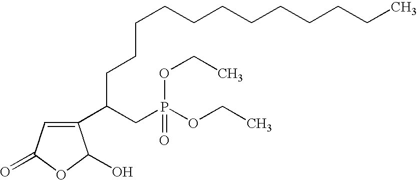 | Aller- gan | | U.S. Pat. No. 5013850 |
| 969 | 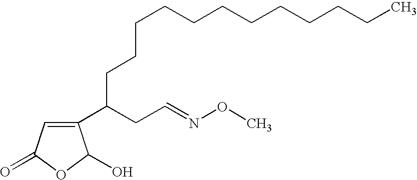 | Aller- gan | | U.S. Pat. No. 5013850 |
| 970 | 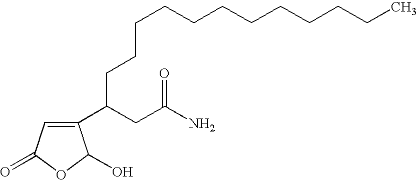 | Aller- gan | | U.S. Pat. No. 5013850 |
| 971 | 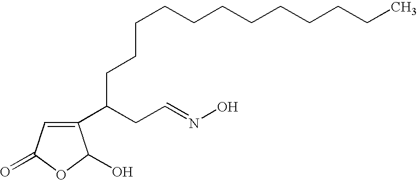 | Aller- gan | | U.S. Pat. No. 5013850 |
| 972 | 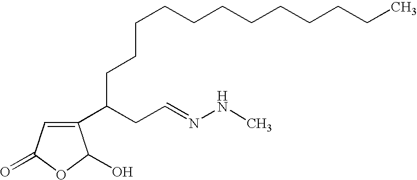 | Aller- gan | | U.S. Pat. No. 5013850 |
| 973 | 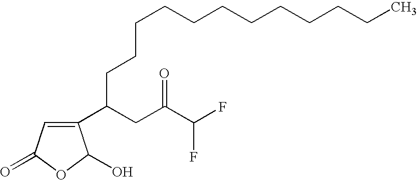 | Aller- gan | | U.S. Pat. No. 5013850 |
| 974 | 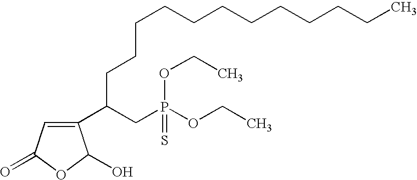 | Aller- gan | | U.S. Pat. No. 5013850 |
| 975 | 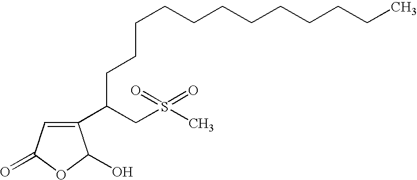 | Aller- gan | | U.S. Pat. No. 5013850 |
| 976 | 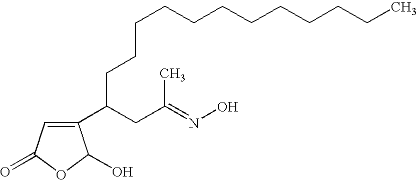 | Aller- gan | | U.S. Pat. No. 5013850 |
| 977 | 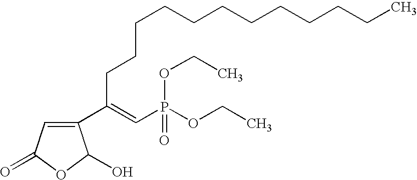 | Aller- gan | | U.S. Pat. No. 5013850 |
| 978 | 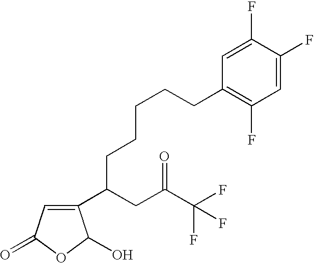 | Aller- gan | | U.S. Pat. No. 5013850 |
| 979 | 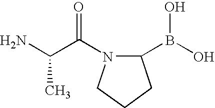 | New England Med. Center Hosp. | | WO 9116339 |
| 980 | 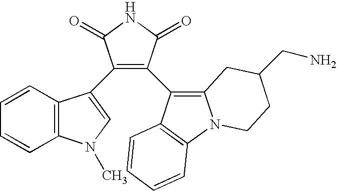 | Roche | 1) Hill, C. H. 6th SCI-RSC Med Chem Symp (Sept 8-11, Cam- bridge) 1991, Abst S18. | EP 384349 |
| 981 | 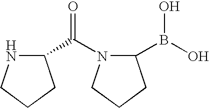 | New England Med. Center Hosp. | | WO 9116339 |
| 982 | 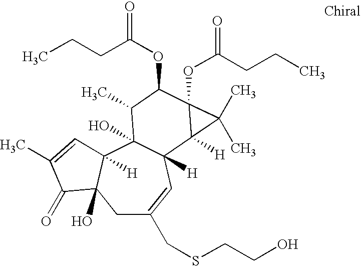 | Adler | | U.S. Pat. No. 5145842 |
| 983 | 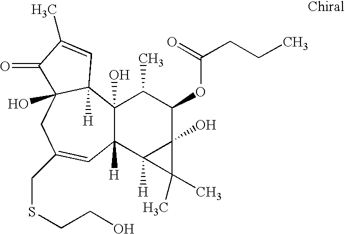 | Adler | | U.S. Pat. No. 5145842 |
| 984 | 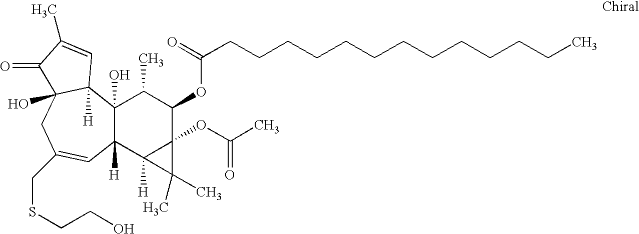 | Adler | | U.S. Pat. No. 5145842 |
| 985 | 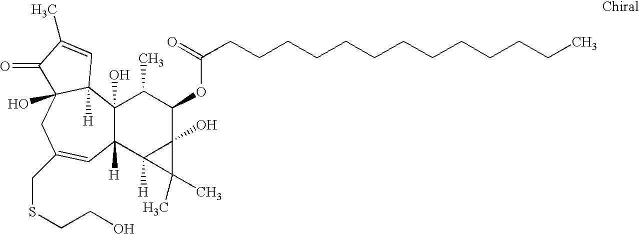 | Adler | | U.S. Pat. No. 5145842 |
| 986 | 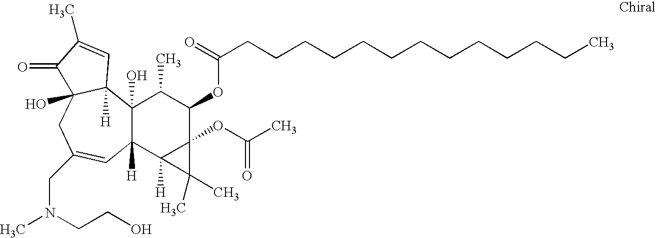 | Adler | | U.S. Pat. No. 5145842 |
| 987 | 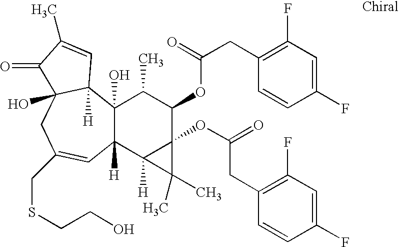 | Adler | | U.S. Pat. No. 5145842 |
| 988 | 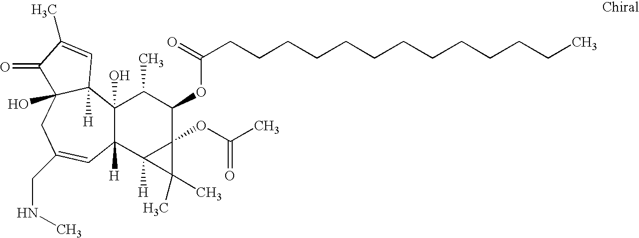 | Adler | | U.S. Pat. No. 5145842 |
| 989 | 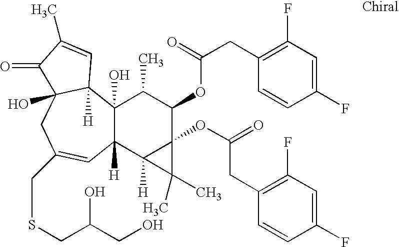 | Adler | | U.S. Pat. No. 5145842 |
| 990 | 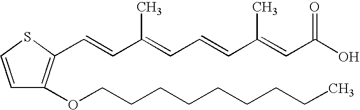 | Roche | | EP 510473 |
| 991 | 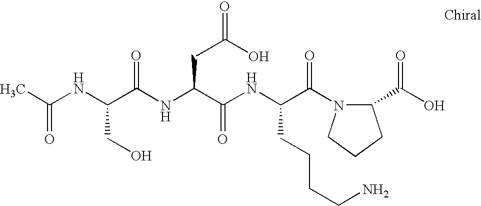 | Beau- four- Ipsen | 1) Carde, P. et al. Proc Amer Soc Clin Oncol 1991, 10: Abst 324. | AU 8810261 |
| 992 | 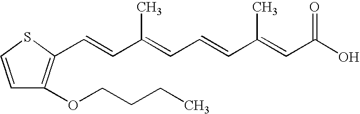 | Roche | | EP 510473 |
| 993 | 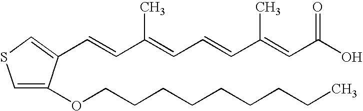 | Roche | | EP 510473 |
| 994 | 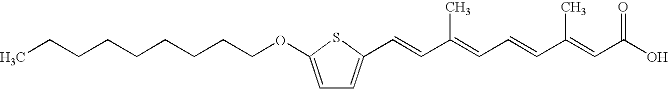 | Roche | | EP 510473 |
| 995 | 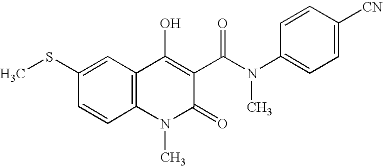 | Fuji- sawa | | WO 9218483 |
| 996 | 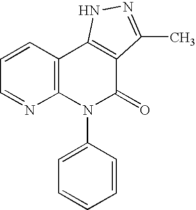 | Kyowa Hakko | | EP 526840 |
| 997 | 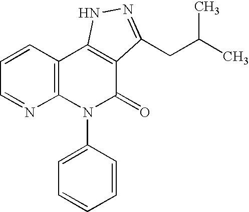 | Kyowa Hakko | | EP 526840 |
| 998 | 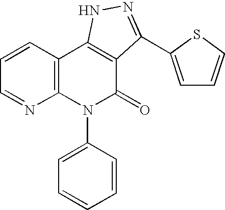 | Kyowa Hakko | | EP 526840 |
| 999 | 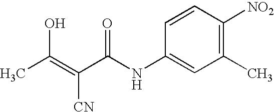 | Aventis Pharma | | EP 538783 |
| 1000 | 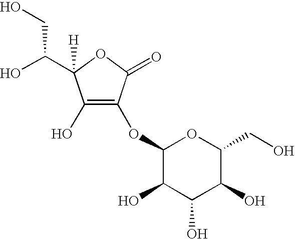 | Haya- shibara | 1) Yama- moto, I. et al. 18th Int Cong Chemother (June 27-July 2, Stockholm) 1993, Abst 516. | EP 539196 |
| 1001 | 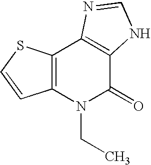 | Kyowa Hakko | | WO 9312116 |
| 1002 | 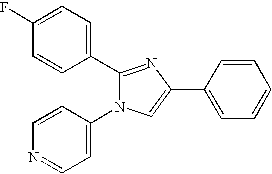 | Glaxo Smith Kline | | WO 9314082 |
| 1003 | 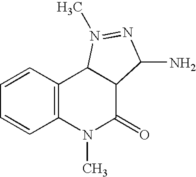 | Otsuka | | JP 93132484 |
| 1004 | 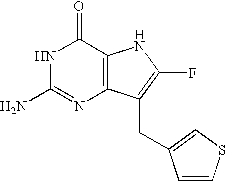 | Pfizer | | U.S. Pat. No. 5236926 |
| 1005 | 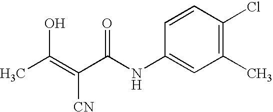 | Aventis Pharma | | EP 538783 |
| 1006 | 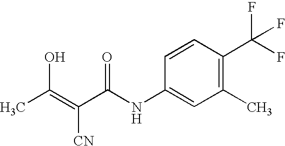 | Aventis Pharma | | EP 538783 |
| 1007 | 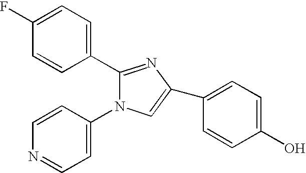 | Glaxo Smith Kline | | WO 9314082 |
| 1008 | 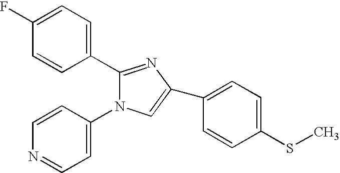 | Glaxo Smith Kline | | WO 9314082 |
| 1009 | 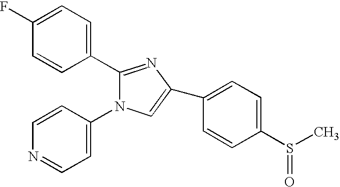 | Glaxo Smith Kline | | WO 9314082 |
| 1010 | 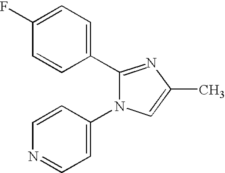 | Glaxo Smith Kline | | WO 9314082 |
| 1011 | 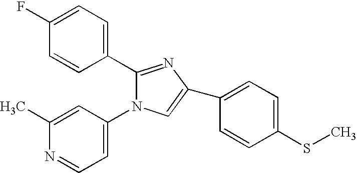 | Glaxo Smith Kline | | WO 9314082 |
| 1012 | 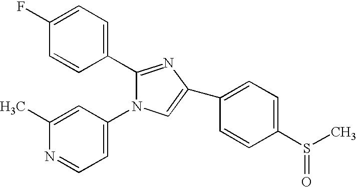 | Glaxo Smith Kline | | WO 9314082 |
| 1013 | 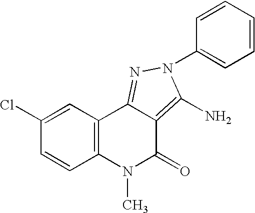 | Otsuka | | JP 93132484 |
| 1014 | 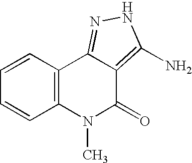 | Otsuka | | JP 93132484 |
| 1015 | 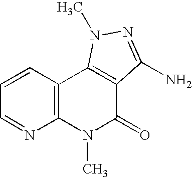 | Otsuka | | JP 93132484 |
| 1016 | 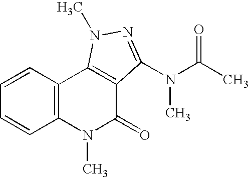 | Otsuka | | JP 93132484 |
| 1017 | 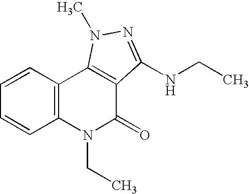 | Otsuka | | JP 93132484 |
| 1018 | 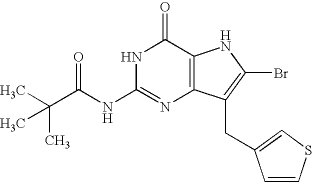 | Pfizer | | U.S. Pat. No. 5236926 |
| 1019 | 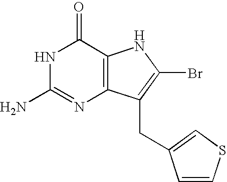 | Pfizer | | U.S. Pat. No. 5236926 |
| 1020 | 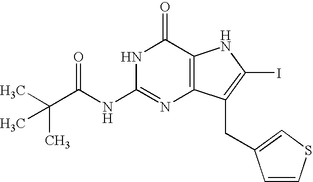 | Pfizer | | U.S. Pat. No. 5236926 |
| 1021 | 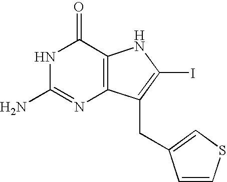 | Pfizer | | U.S. Pat. No. 5236926 |
| 1022 | 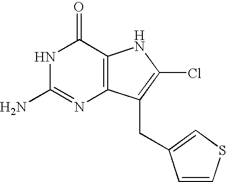 | Pfizer | | U.S. Pat. No. 5236926 |
| 1023 | 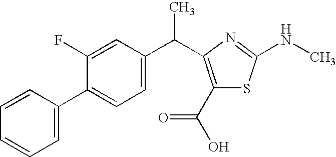 | Sumi- tomo | | U.S. Pat. No. 5258396 |
| 1024 | 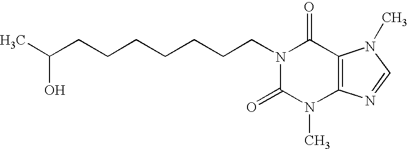 | Cell Thera- peutics | | WO 9411001 |
| 1025 | 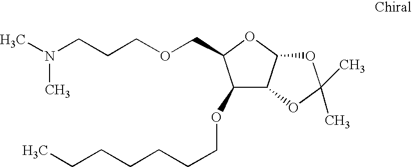 | Green- wich Pharm. | | WO 9411381 |
| 1026 | 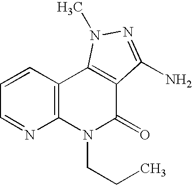 | Otsuka | | JP 94100561 |
| 1027 | 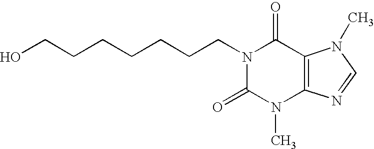 | Cell Thera- peutics | | WO 9411001 |
| 1028 | 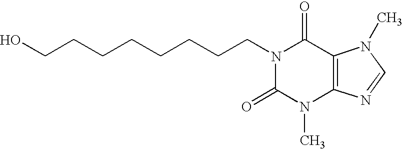 | Cell Thera- peutics | | WO 9411001 |
| 1029 | 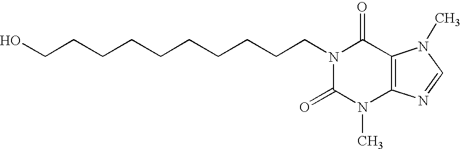 | Cell Thera- peutics | | WO 9411001 |
| 1030 | 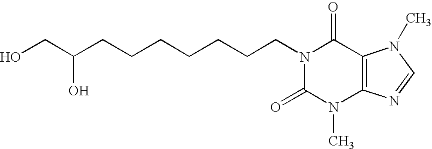 | Cell Thera- peutics | | WO 9411001 |
| 1031 | 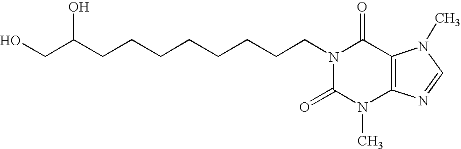 | Cell Thera- peutics | | WO 9411001 |
| 1032 | 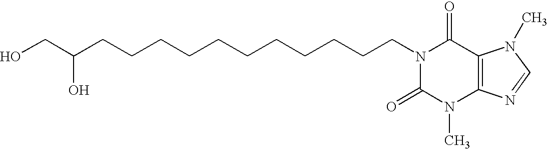 | Cell Thera- peutics | | WO 9411001 |
| 1033 | 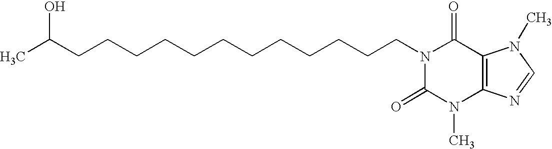 | Cell Thera- peutics | | WO 9411001 |
| 1034 | 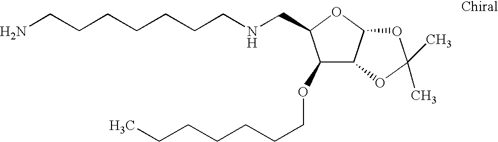 | Green- wich Pharm. | | WO 9411381 |
| 1035 | 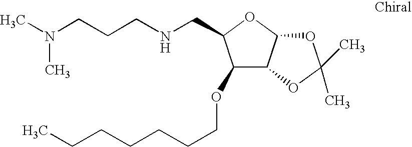 | Green- wich Pharm. | | WO 9411381 |
| 1036 | 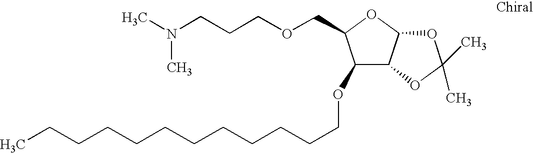 | Green- wich Pharm. | | WO 9411381 |
| 1037 | 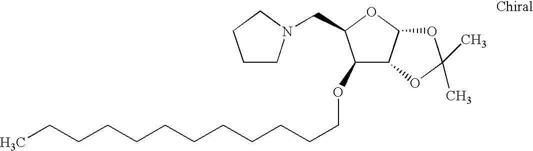 | Green- wich Pharm. | | WO 9411381 |
| 1038 | 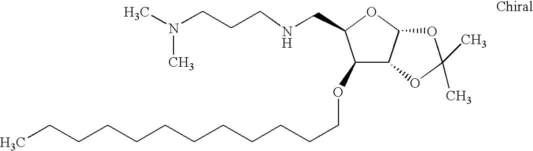 | Green- wich Pharm. | | WO 9411381 |
| 1039 | 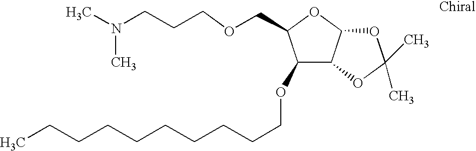 | Green- wich Pharm. | | WO 9411381 |
| 1040 | 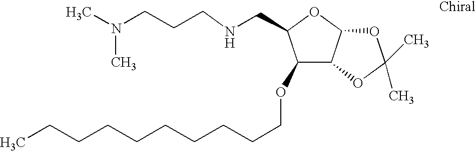 | Green- wich Pharm. | | WO 9411381 |
| 1041 | 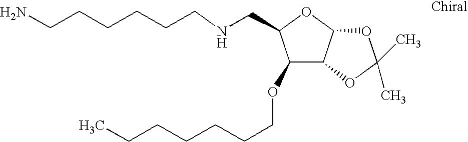 | Green- wich Pharm. | | WO 9411381 |
| 1042 | 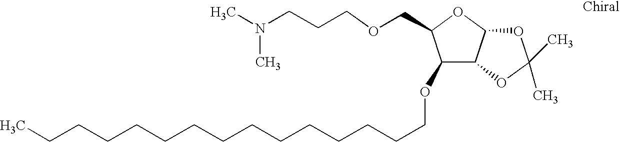 | Green- wich Pharm. | | WO 9411381 |
| 1043 | 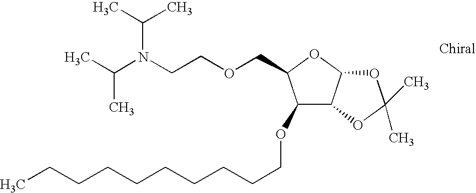 | Green- wich Pharm. | | WO 9411381 |
| 1044 | 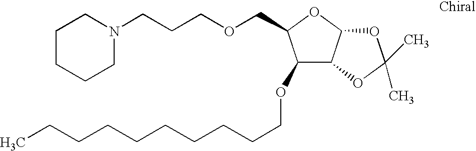 | Green- wich Pharm. | | WO 9411381 |
| 1045 | 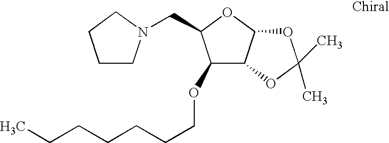 | Green- wich Pharm. | | WO 9411381 |
| 1046 | 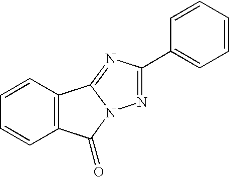 | Bristol Tech- nol. | | GB 2278842 |
| 1047 | 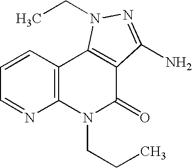 | Otsuka | | JP 94100561 |
| 1048 | 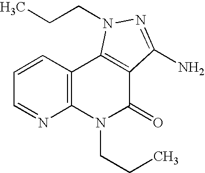 | Otsuka | | JP 94100561 |
| 1049 | 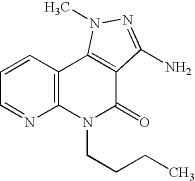 | Otsuka | | JP 94100561 |
| 1050 | 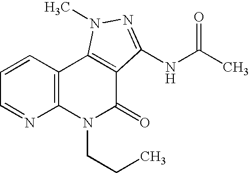 | Otsuka | | JP 94100561 |
| 1051 | 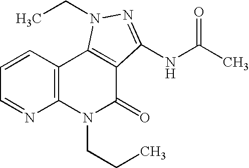 | Otsuka | | JP 94100561 |
| 1052 | 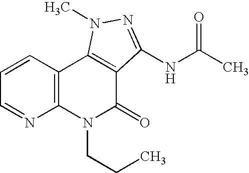 | Otsuka | | JP 94100561 |
| 1053 | 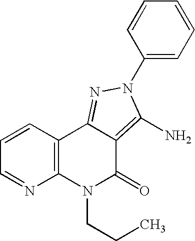 | Otsuka | | JP 94100561 |
| 1054 | 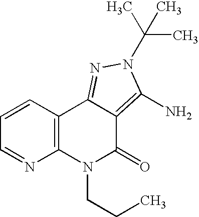 | Otsuka | | JP 94100561 |
| 1055 | 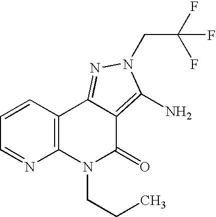 | Otsuka | | JP 94100561 |
| 1056 | 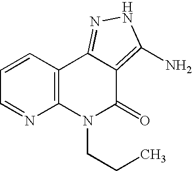 | Otsuka | | JP 94100561 |
| 1057 | 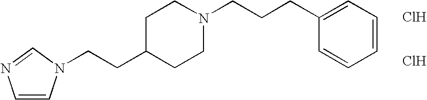 | Abbott GmbH | | WO 9500507 |
| 1058 | 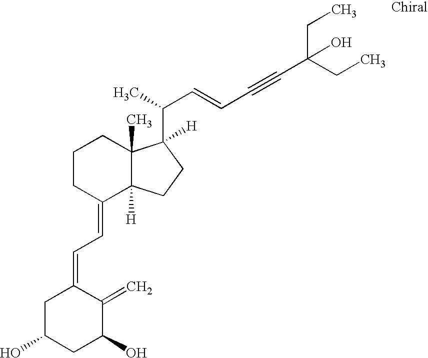 | Leo | | WO 9502577 |
| 1059 | 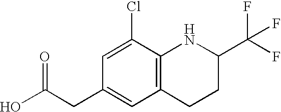 | Kyorin | Kono, Y. et al. 115th Annu Meet Pharmaceut Soc Jpn March 29-31, Sendai) 1995, Abst 30 (A3) 10-3. | EP 538477 |
| 1060 | 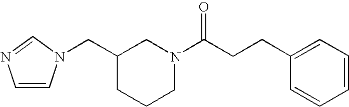 | Abbott GmbH | | WO 9500507 |
| 1061 | 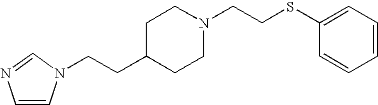 | Abbott GmbH | | WO 9500507 |
| 1062 | 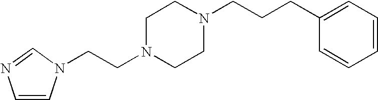 | Abbott GmbH | | WO 9500507 |
| 1063 | 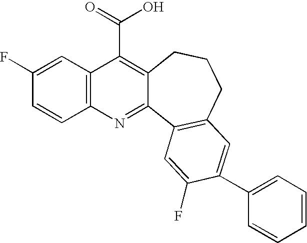 | Kyowa Hakko | | WO 9509153 |
| 1064 | 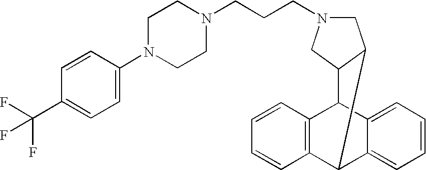 | Bayer | | U.S. Pat. No. 5409932 |
| 1065 | 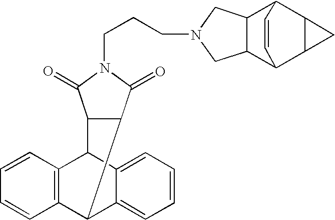 | Bayer | | U.S. Pat. No. 5411960 |
| 1066 | 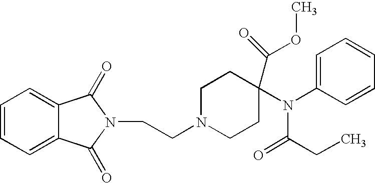 | Baxter | France, C. P. et al. Drug Develop Res 1995, 35:49. | EP 396282 |
| 1067 | 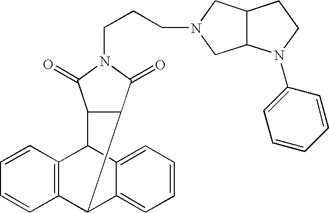 | Bayer | | U.S. Pat. No. 5411960 |
| 1068 | 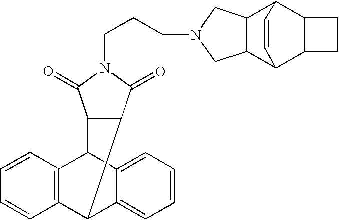 | Bayer | | U.S. Pat. No. 5411960 |
| 1069 | 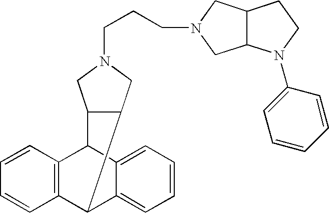 | Bayer | | U.S. Pat. No. 5411960 |
| 1070 | 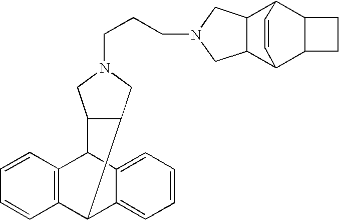 | Bayer | | U.S. Pat. No. 5411960 |
| 1071 | 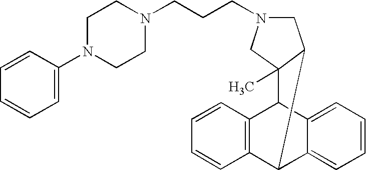 | Bayer | | U.S. Pat. No. 5409932 |
| 1072 | 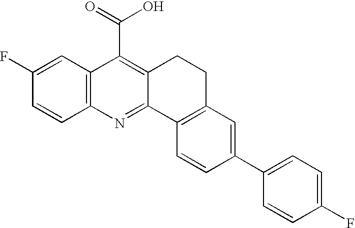 | Kyowa Hakko | | WO 9509153 |
| 1073 | 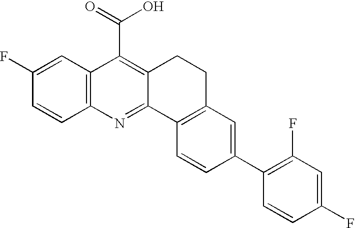 | Kyowa Hakko | | WO 9509153 |
| 1074 | 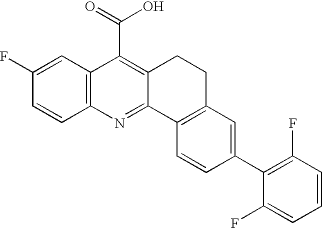 | Kyowa Hakko | | WO 9509153 |
| 1075 | 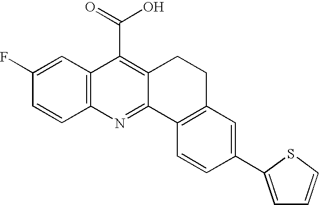 | Kyowa Hakko | | WO 9509153 |
| 1076 | 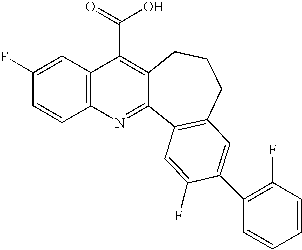 | Kyowa Hakko | | WO 9509153 |
| 1077 | 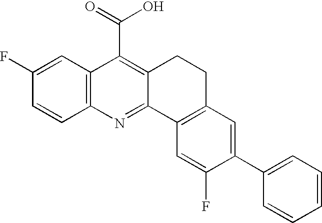 | Kyowa Hakko | | WO 9509153 |
| 1078 | 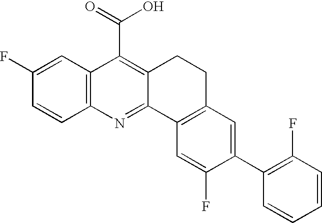 | Kyowa Hakko | | WO 9509153 |
| 1079 | 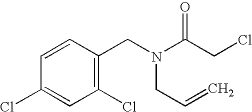 | Sanofi- Synthe- labo | | WO 9529672 |
| 1080 | 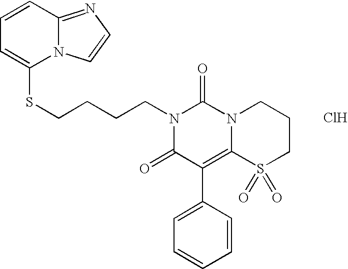 | Takeda | | WO 9535296 |
| 1081 | 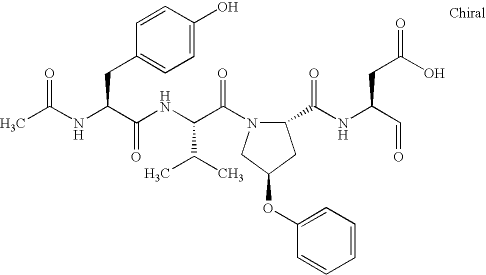 | Vertex | | WO 9535308 |
| 1082 | 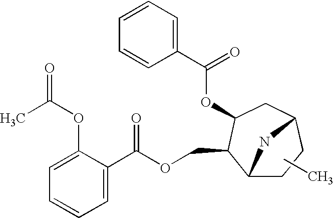 | Entro pin | | WO 9534561 |
| 1083 | 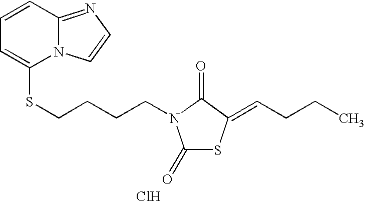 | Takeda | | WO 9535296 |
| 1084 | 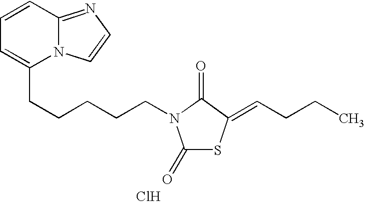 | Takeda | | WO 9535296 |
| 1085 | 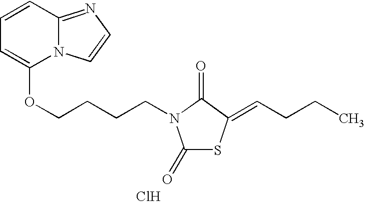 | Takeda | | WO 9535296 |
| 1086 | 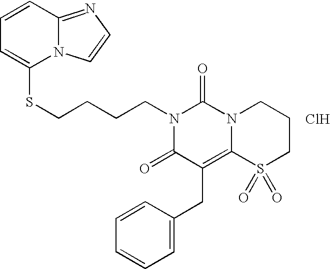 | Takeda | | WO 9535296 |
| 1087 | 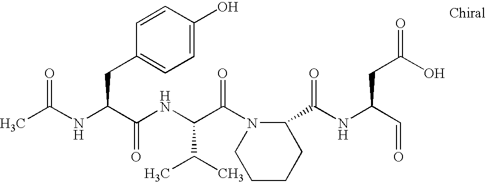 | Vertex | | WO 9535308 |
| 1088 | 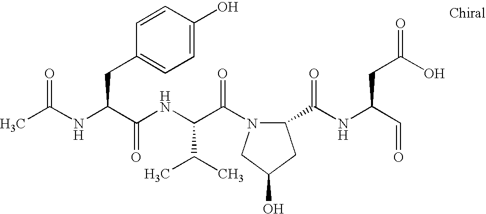 | Vertex | | WO 9535308 |
| 1089 | 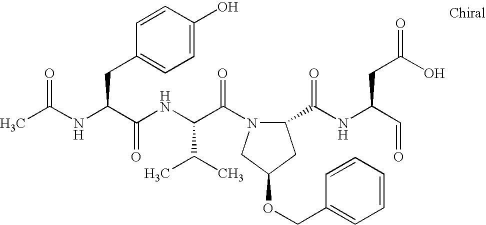 | Vertex | | WO 9535308 |
| 1090 | 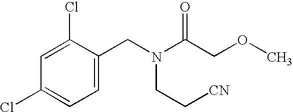 | Sanofi- Synthe- labo | | WO 9529672 |
| 1091 | 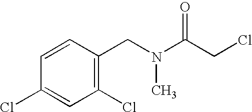 | Sanofi- Synthe- labo | | WO 9529672 |
| 1092 | 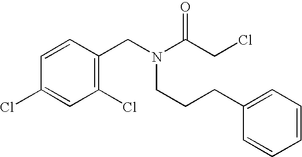 | Sanofi- Synthe- labo | | WO 9529672 |
| 1093 | 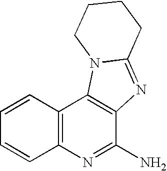 | 3M Pharma- ceuti cals | | U.S. Pat. No. 5482936 |
| 1094 | 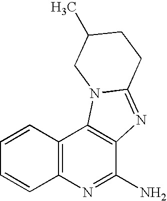 | 3M Pharma- ceuti cals | | U.S. Pat. No. 5482936 |
| 1095 | 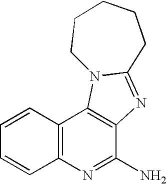 | 3M Pharma- ceuti cals | | U.S. Pat. No. 5482936 |
| 1096 | 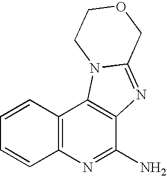 | 3M Pharma- ceuti cals | | U.S. Pat. No. 5482936 |
| 1097 | 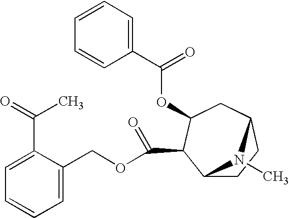 | Entro- pin | | WO 9534561 |
| 1098 | 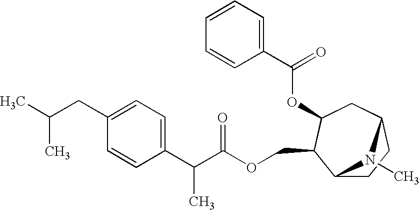 | Entro- pin | | WO 9534561 |
| 1099 | 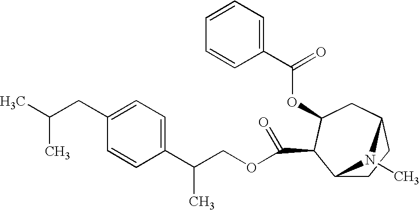 | Entro- pin | | WO 9534561 |
| 1100 | 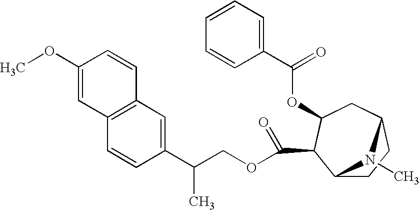 | Entro- pin | | WO 9534561 |
| 1101 | 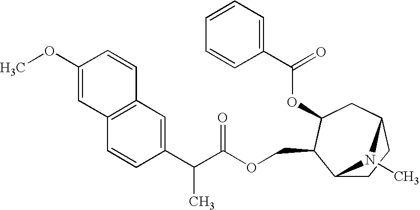 | Entro- pin | | WO 9534561 |
| 1102 | 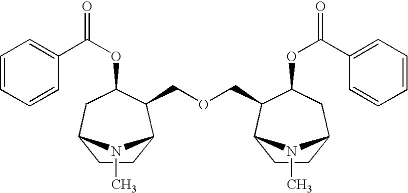 | Entro- pin | | WO 9534561 |
| 1103 | 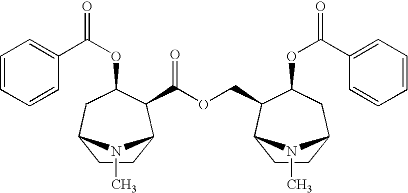 | Entro- pin | | WO 9534561 |
| 1104 | 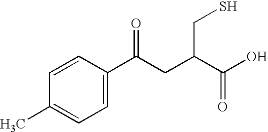 | Taisho | 1) Yoshida, H. et al. Biol Pharm Bull 1997, 20(1): 94. | JP 93051358 |
| 1105 | 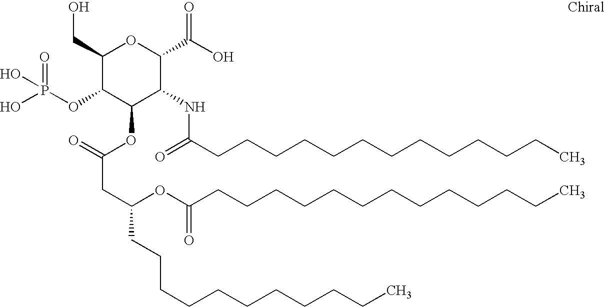 | Sankyo | Shiozaki, M. et al. Tetrahedron Lett 1996 37(40): 7271. | |
| 1106 | 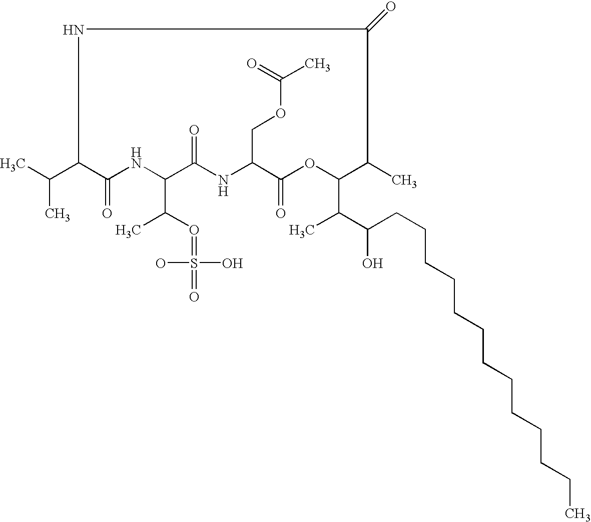 | Nippon Kayaku | | JP 96283290 |
| 1107 | 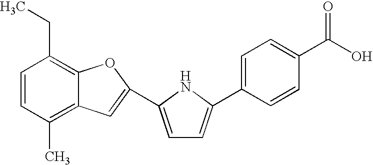 | Eisai | | EP 889032 |
| 1108 | 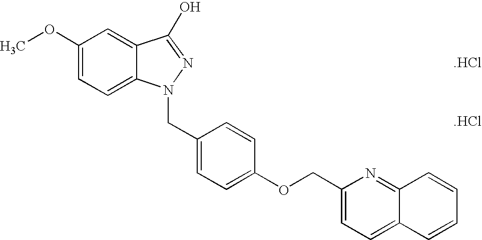 | AWD. pharma | | WO 9734874 |
| 1109 | 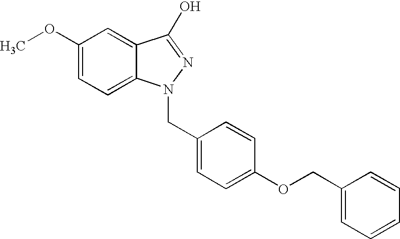 | AWD. pharma | | WO 9734874 |
| 1110 | 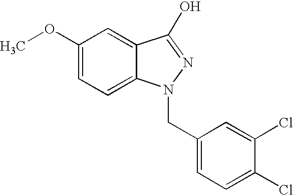 | AWD. pharma | | WO 9734874 |
| 1111 | 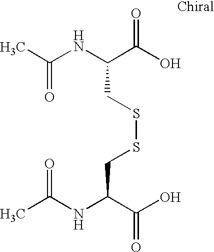 | Astra Zeneca | 1) Saem- strand, B. et al. J Pharm- acol Exp Ther 1999, 228(3): 1174. | EP 0463514 |
| 1112 | 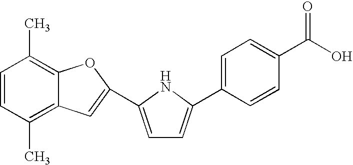 | Eisai | Nagai, M. et al. 217th ACS Natl Meet (March 21-25, Ana- heim) 1999, Abst MEDI 050. | EP 889032 |
| 1113 | 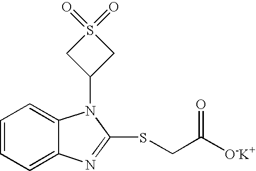 | Bashkir Medical Univer- sity | Sadykov, R. F. et al. Naunyn- Schmied Arch Pharm- acol 1998, 358(1, Suppl. 2): Abst P 52.28. | |
| 1114 | 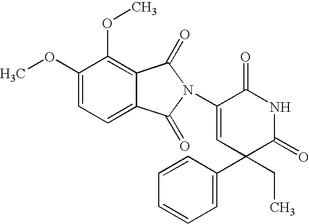 | Gruen- enthal | | EP 856513 |
| 1115 | 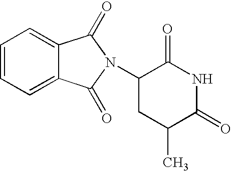 | Gruen- enthal | | EP 856513 |
| 1116 | 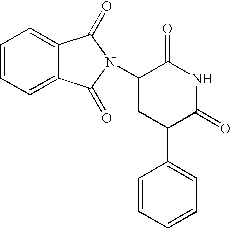 | Gruen- enthal | | EP 856513 |
| 1117 | 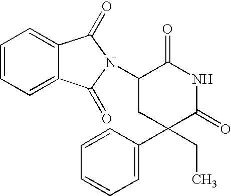 | Gruen- enthal | | EP 856513 |
| 1118 | 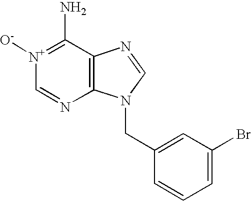 | Japan Energy | | JP 98231297 |
| 1119 | 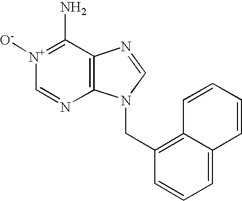 | Japan Energy | | JP 98231297 |
| 1120 | 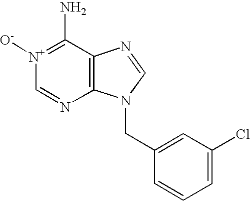 | Japan Energy | | JP 98231297 |
| 1121 | 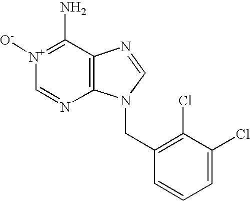 | Japan Energy | | JP 98231297 |
| 1122 | 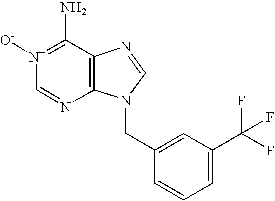 | Japan Energy | | JP 98231297 |
| 1123 | 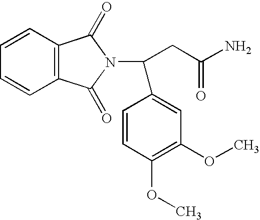 | Celgene | 1) Moreira, A. L. et al. J Neuro- Oncol 1999, 43(2): 109. | U.S. Pat. No. 5463063 |
| 1124 | 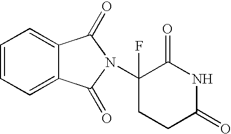 | Celgene | | U.S. Pat. No. 5874448 |
| 1125 | 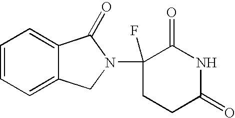 | Celgene | | U.S. Pat. No. 5874448 |
| 1126 | 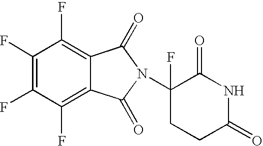 | Celgene | | U.S. Pat. No. 5874448 |
| 1127 | 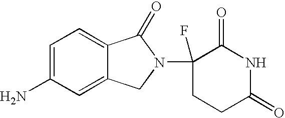 | Celgene | | U.S. Pat. No. 5874448 |
| 1128 | 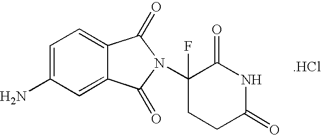 | Celgene | | U.S. Pat. No. 5874448 |
| 1129 | 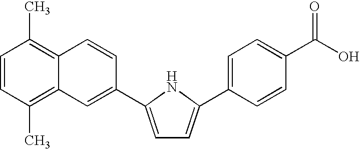 | Eisai | | EP 889032 |
| 1130 | 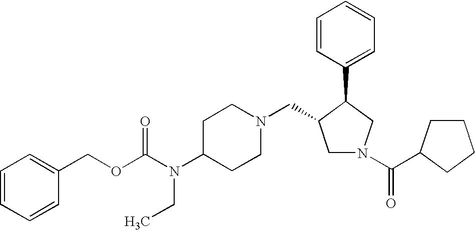 | Merck & Co. | | WO 9909984 |
| 1131 | 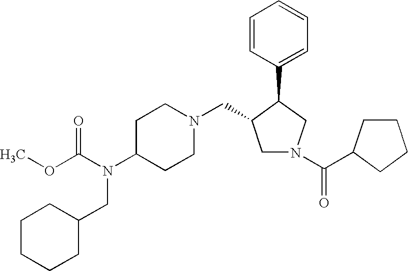 | Merck & Co. | | WO 9909984 |
| 1132 | 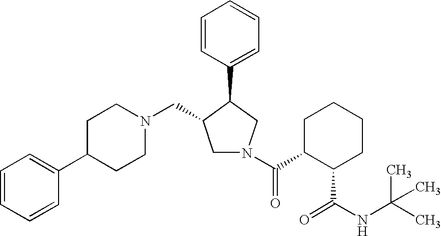 | Merck & Co. | | WO 9909984 |
| 1133 | 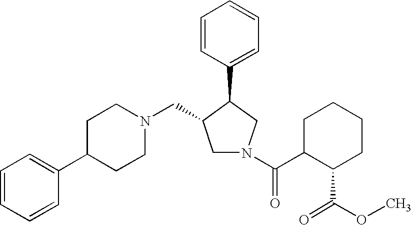 | Merck & Co. | | WO 9909984 |
| 1134 | 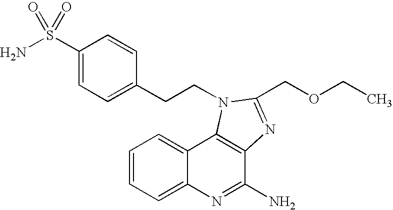 | Hokur- iku | | JP 99080156 |
| 1135 | 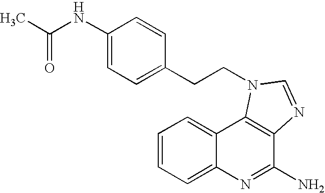 | Hokur- iku | | JP 99080156 |
| 1136 | 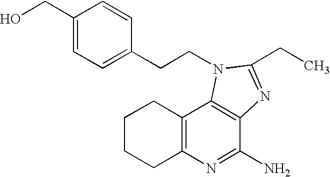 | Hokur- iku | | JP 99080156 |
| 1137 | 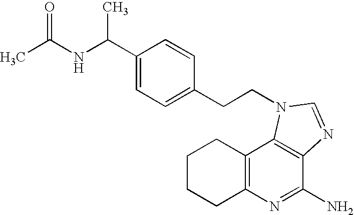 | Hokur- iku | | JP 99080156 |
| 1138 | 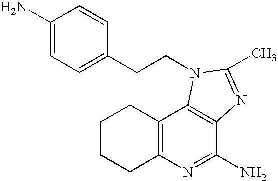 | Hokur- iku | | JP 99080156 |
| 1139 | 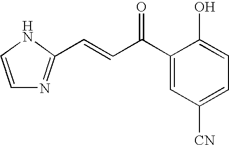 | SSP | | CA 2255337 |
| 1140 | 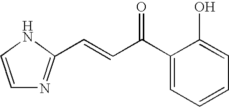 | SSP | | CA 2255337 |
| 1141 | 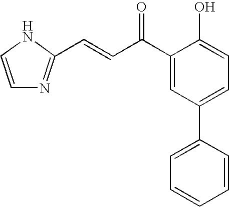 | SSP | | CA 2255337 |
| 1142 | 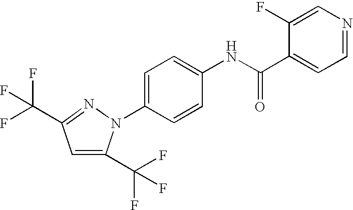 | Abbott | Madar, D. et al. 222nd ACS Natl Meet (Aug 26-30, Chicago) 2001, Abst MEDI 7. | EP 1068187 |
| 1143 | 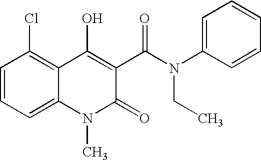 | Active Biotech | | WO 9955678 |
| 1144 | 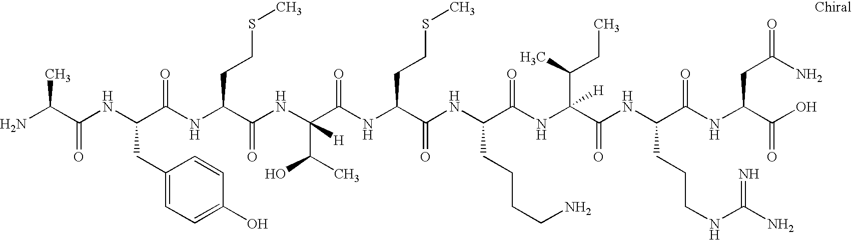 | Amer- sham | 1) Gesser, B. et al. Proc Natl Acad Sci USA 1997, 94(26): 14620. | WO 9601318 |
| 1145 | 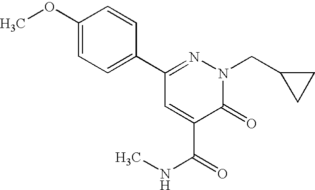 | Kowa | | WO 9944995 |
| 1146 | 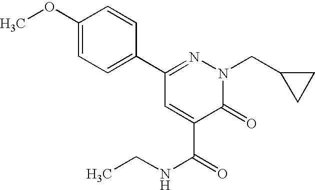 | Kowa | | WO 9944995 |
| 1147 | 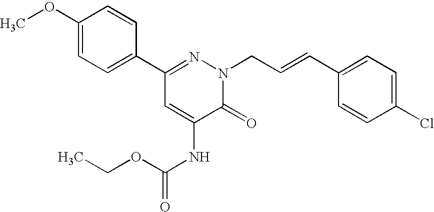 | Kowa | | WO 9944995 |
| 1148 | 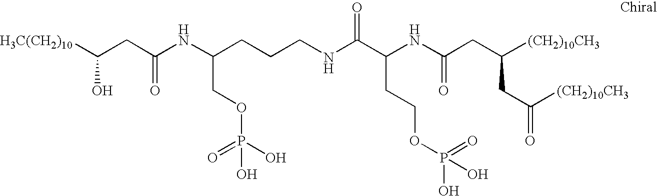 | OM Pharma | | WO 0000462 |
| 1149 | 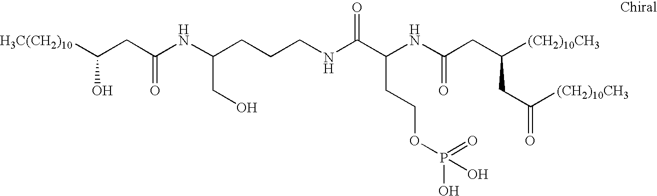 | OM Pharma | | WO 0000462 |
| 1150 | 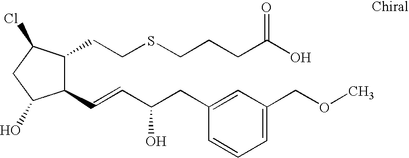 | Ono | | WO 0003980 |
| 1151 | 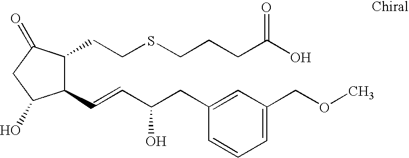 | Ono | | WO 0003980 |
| 1152 | 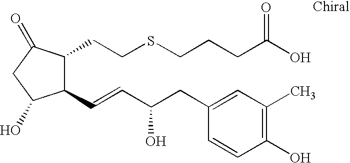 | Ono | | WO 0003980 |
| 1153 | 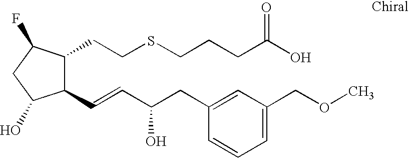 | Ono | | WO 0003980 |
| 1154 | 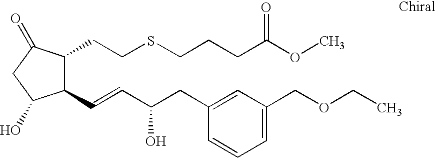 | Ono | | WO 0003980 |
| 1155 | 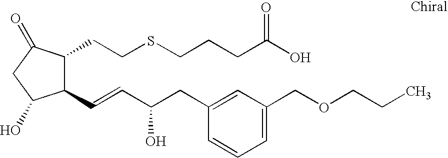 | Ono | | WO 0003980 |
| 1156 | 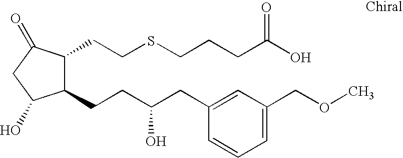 | Ono | | WO 0003980 |
| 1157 | 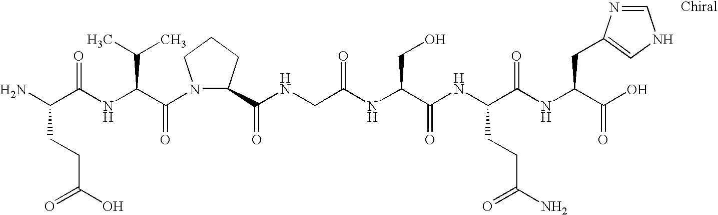 | Univer- sity of Bristol | | WO 0014114 |
| 1158 | 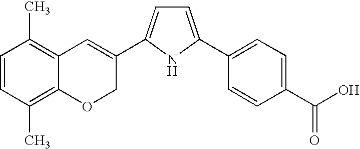 | Eisai | Hibi, S. et al. Bioorg Med Chem Lett 2000, 10(7): 623. | EP 0889032 |
| 1159 | 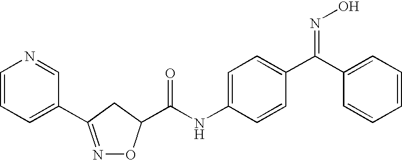 | Janssen | | WO 0021959 |
| 1160 | 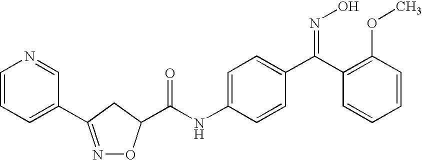 | Janssen | | WO 0021959 |
| 1161 | 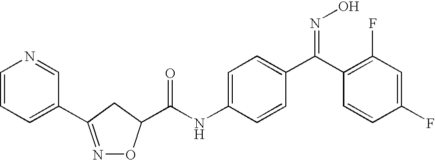 | Janssen | | WO 0021959 |
| 1162 | 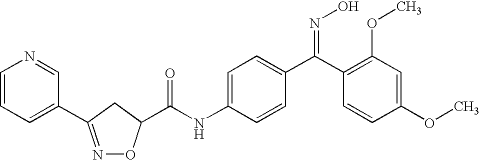 | Janssen | | WO 0021959 |
| 1163 | 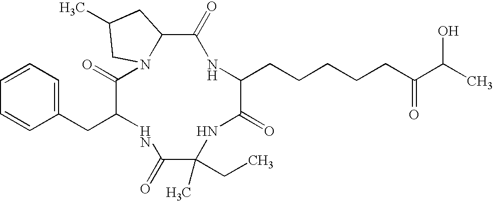 | Fuji- sawa | | WO 0021979 |
| 1164 | 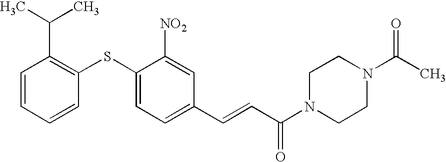 | Abbott | 1) Liu, G. et al. 220th ACS Natl Meet (Aug 20-24, Washington DC) 2000, Abst MEDI 171. | 1) U.S. Pat. No. 6110922 |
| 1165 | 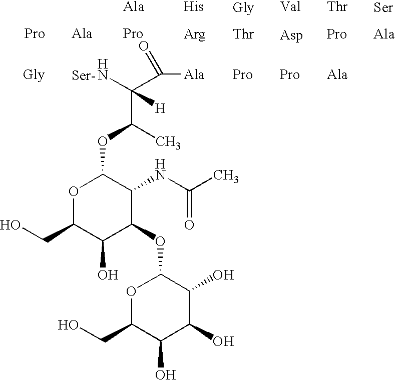 | Cancer Re- search UK | | WO 0052046 |
| 1166 | 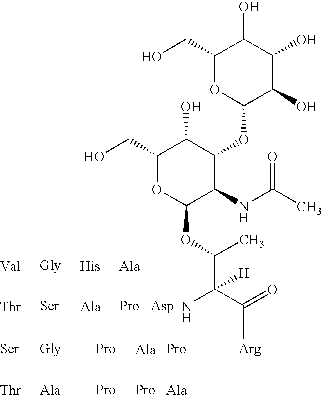 | Cancer Re- search UK | | WO 0052046 |
| 1167 | 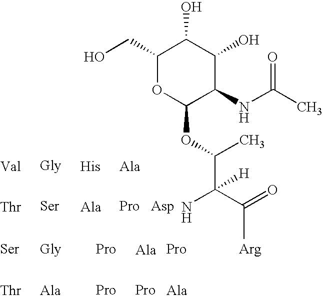 | Cancer Re- search UK | | WO 0052046 |
| 1168 | 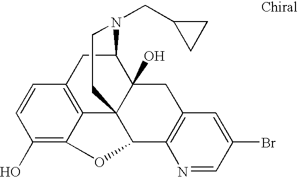 | South- ern Re- search Institute | | WO 0112197 |
| 1169 | 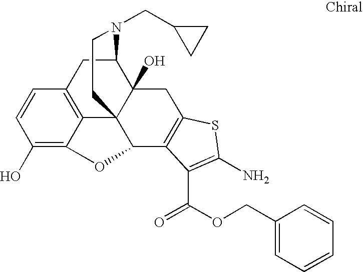 | South- ern Re- search Institute | | WO 0112197 |
| 1170 | 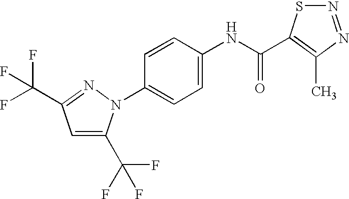 | Abbott Labs. | | EP 1068187 |
| 1171 | 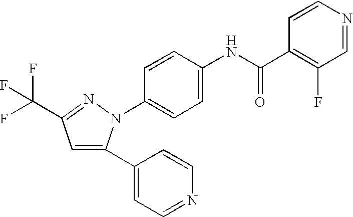 | Abbott | | EP 1068187 |
| 1172 | 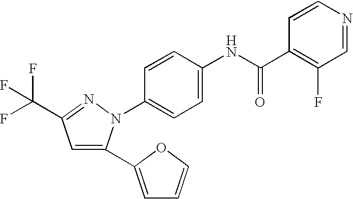 | Abbott | | EP 1068187 |
| 1173 | 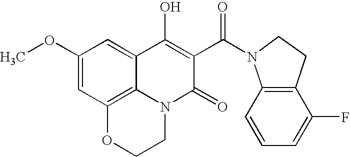 | Fuji- sawa | Spears, G. et al. 21st Symp Med Chem (Nov 28-30, Kyoto) 2001, Abst 2P-29. | |
| 1174 | 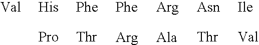 | Austin Re- search Institute | Tselios, T. et al. J Med Chem 2002, 45(2): 275. | |
| 1175 | 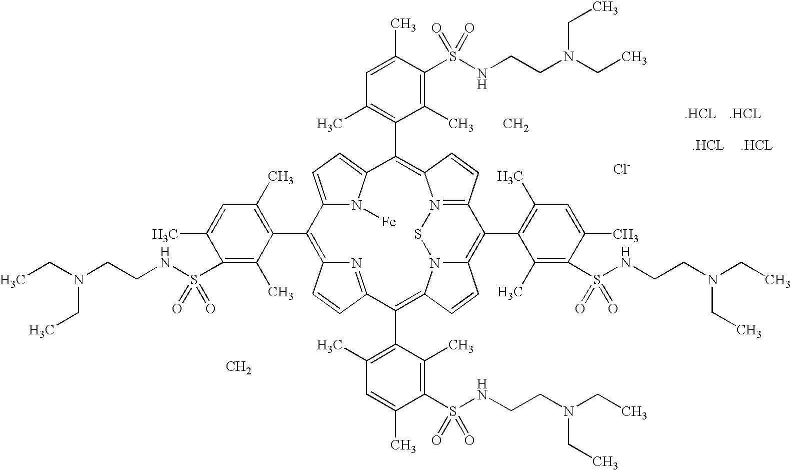 | Eukar- ion | | WO 0204454 |
| 1176 | 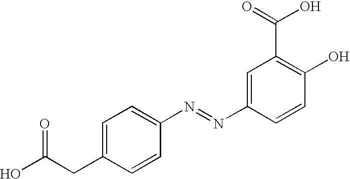 | Nobex | 1) Riggs- Sauthier, J. A. et al. 224th ACS Natl Meet (Aug 18-22, Boston) 2002, Abst MEDI 305. | WO 0218324 |
| 1177 | 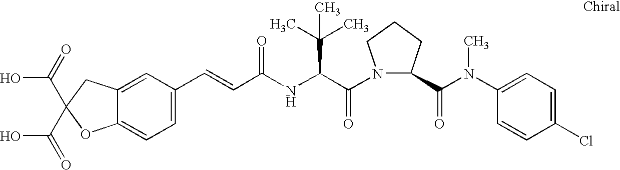 | Tularik | | WO 0238107 |
| 1178 | 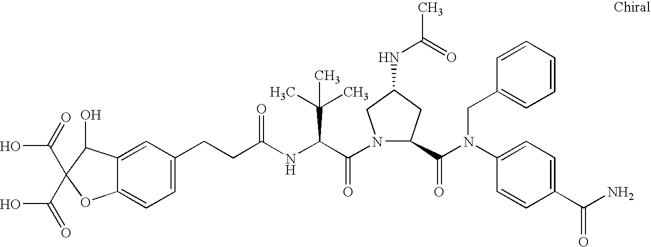 | Tularik | | WO 0238107 |
| 1179 | 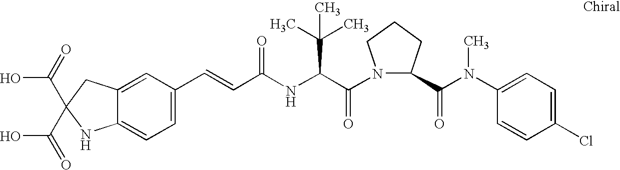 | Tularik | | WO 0238107 |
| 1180 | 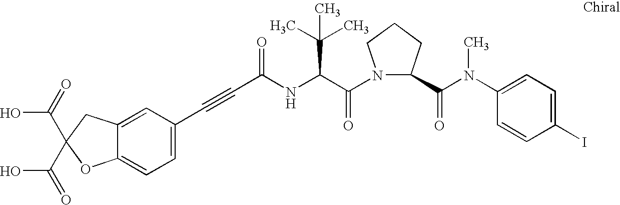 | Tularik | | WO 0238107 |
| 1181 | 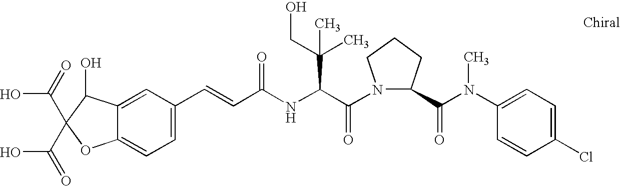 | Tularik | | WO 0238107 |
| 1182 | 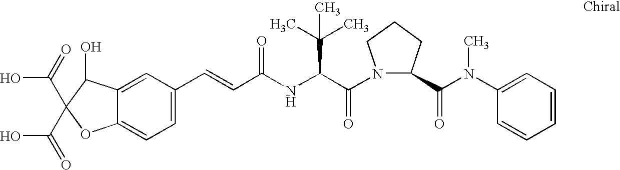 | Tularik | | WO 0238107 |
| 1183 | 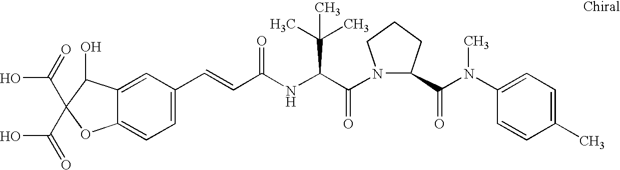 | Tularik | | WO 0238107 |
| 1184 | 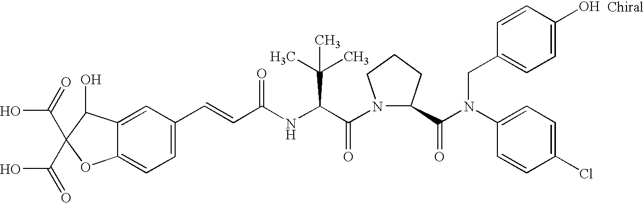 | Tularik | | WO 0238107 |
| 1185 | 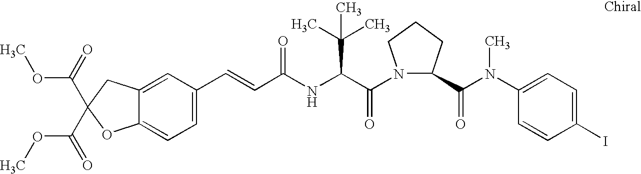 | Tularik | | WO 0238107 |
| 1186 | 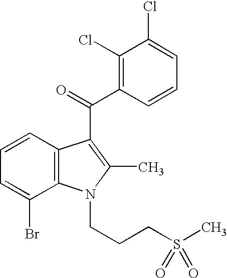 | Sanofi- Synthe labo | | WO 0242269 |
| 1187 | 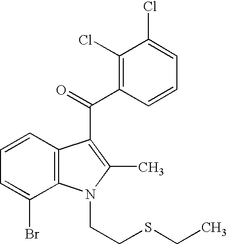 | Sanofi- Synthe labo | | WO 0242269 |
| 1188 | 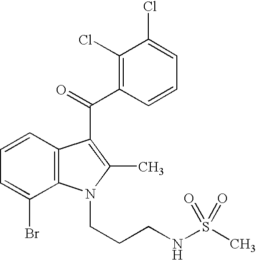 | Sanofi- Synthe labo | | WO 0242269 |
| 1189 | 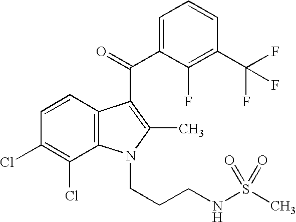 | Sanofi- Synthe labo | | WO 0242269 |
| 1190 | 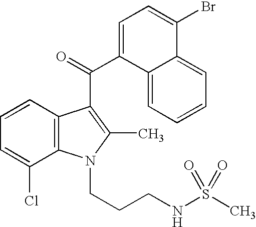 | Sanofi- Synthe labo | | WO 0242269 |
| 1191 | 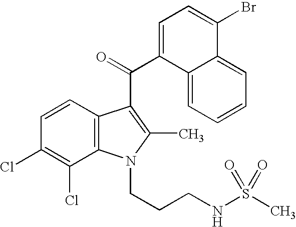 | Sanofi- Synthe labo | | WO 0242269 |
| 1192 | 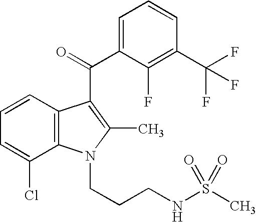 | Sanofi- Synthe labo | | WO 0242269 |
| 1193 | 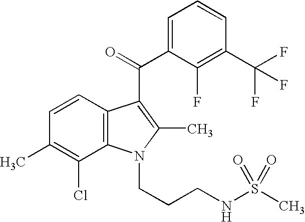 | Sanofi- Synthe labo | | WO 0242269 |
| 1194 | 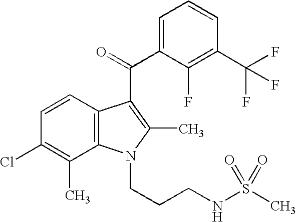 | Sanofi- Synthe labo | | WO 0242269 |
| 1195 | 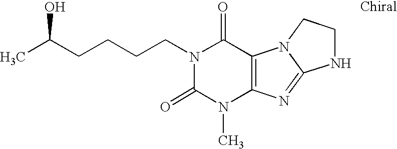 | Cell Thera- peutics | | WO 0268421 |
| 1196 | 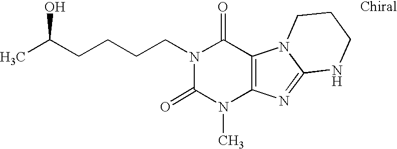 | Cell Thera- peutics | | WO 0268421 |
| 1197 | 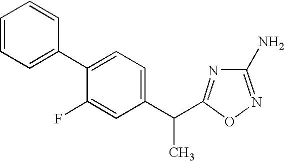 | Sumi- tomo Pharma- ceuti cals | | EP 248399 |
| 1198 | 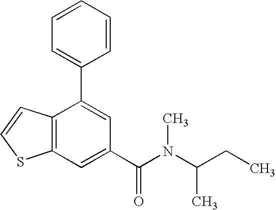 | Aventis Pharma | | EP 248734 |
| 1199 | 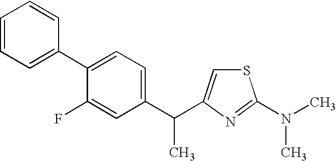 | Sumi- tomo Pharma- ceuti cals | | EP 248399 |
| 1200 | 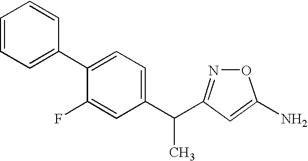 | Sumi- tomo Pharma- ceuti cals | | EP 248399 |
| 1201 | 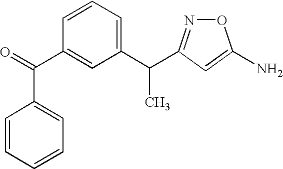 | Sumi- tomo Pharma- ceuti cals | | EP 248399 |
| 1202 | 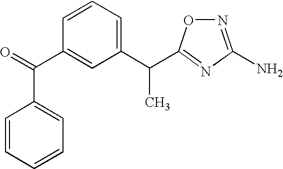 | Sumi- tomo Pharma- ceuti cals | | EP 248399 |
| 1203 | 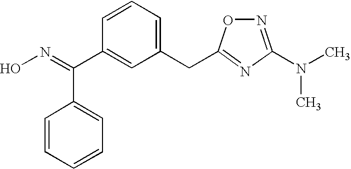 | Sumi- tomo Pharma- ceuti cals | | EP 248399 |
| 1204 | 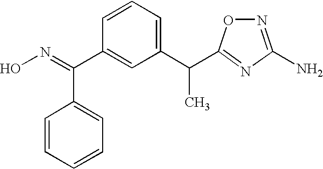 | Sumi- tomo Pharma- ceuti cals | | EP 248399 |
| 1205 | 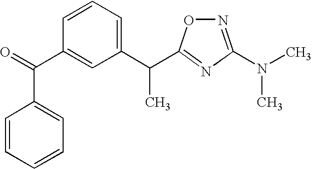 | Sumi- tomo Pharma- ceuti cals | | EP 248399 |
| 1206 | 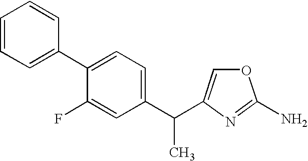 | Sumi- tomo Pharma- ceuti cals | | EP 248399 |
| 1207 | 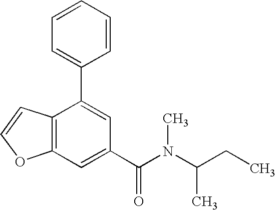 | Aventis Pharma | | EP 248734 |
| 1208 | 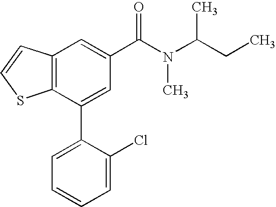 | Aventis Pharma | | EP 248734 |
| 1209 | 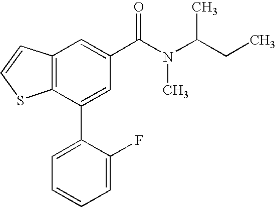 | Aventis Pharma | | EP 248734 |
| 1210 | 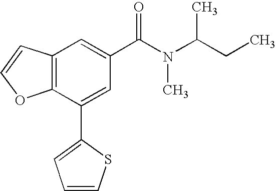 | Aventis Pharma | | EP 248734 |
| 1211 | 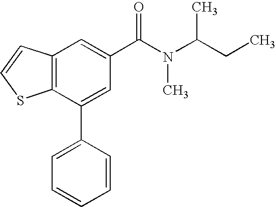 | Aventis Pharma | | EP 248734 |
| 1212 | 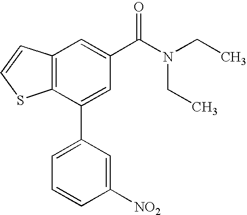 | Aventis Pharma | | EP 248734 |
| 1213 | 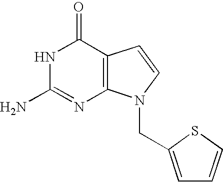 | Pfizer | | AU 8783281 |
| 1214 | 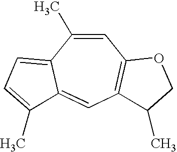 | Harbor Branch Found. | | U.S. Pat. No. 4755529 |
| 1215 | 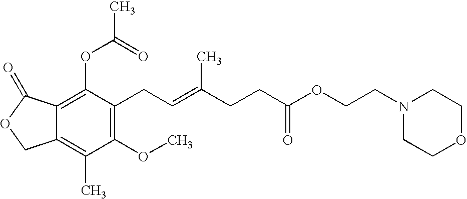 | Roche Bio- science | | AU 8782540 |
| 1216 | 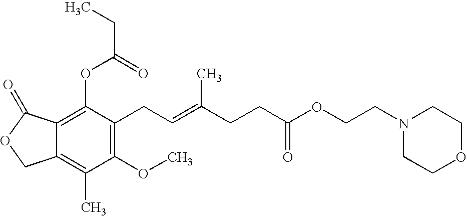 | Roche Bio- science | | AU 8782540 |
| 1217 | 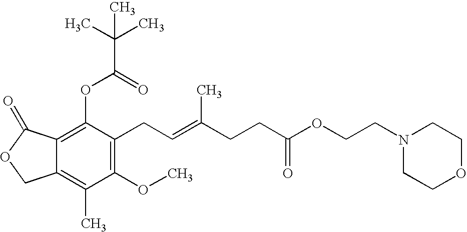 | Roche Bio- science | | AU 8782540 |
| 1218 | 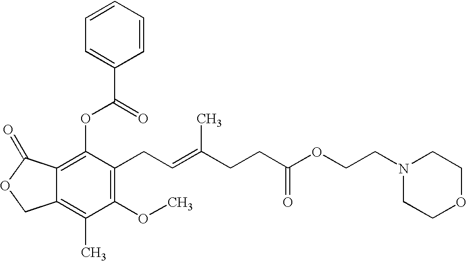 | Roche Bio- science | | AU 8782540 |
| 1219 | 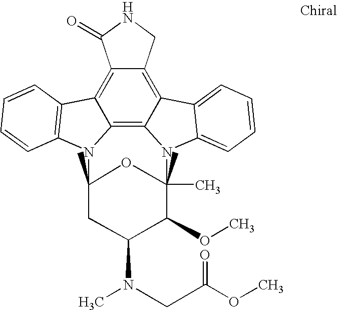 | Novar- tis | | EP 296110 |
| 1220 | 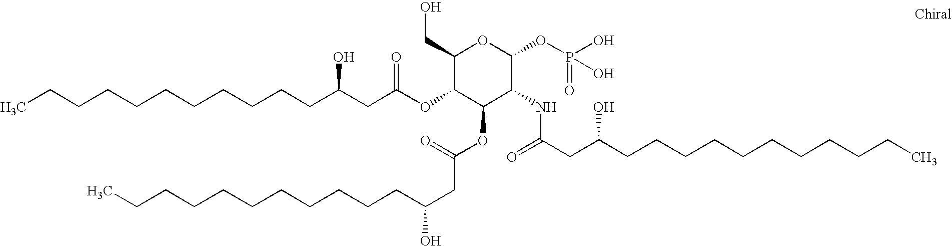 | Novar- tis | 1) Lam, C. et al. Antimicrob Agents Chemother 1991, 35(3): 500. | AU 8822785 |
| 1221 | 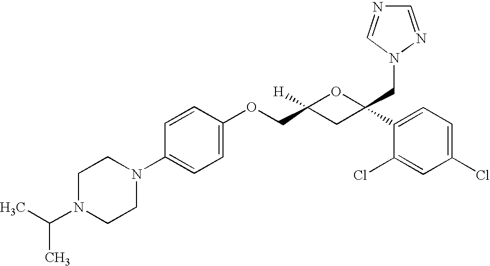 | Scher- ing- Plough | | EP 318214 |
| 1222 | 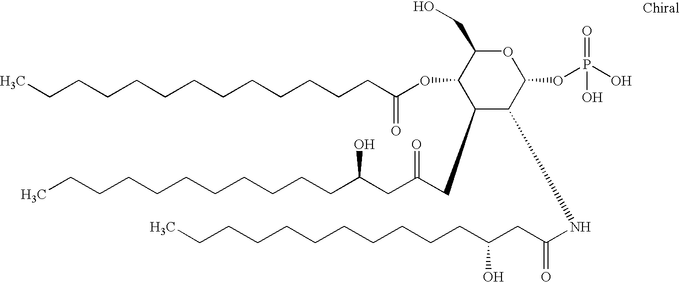 | Novar- tis | | AU 8822785 |
| 1223 | 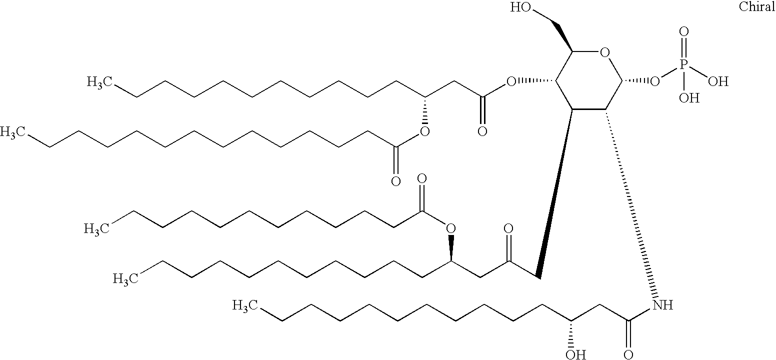 | Novar- tis | | AU 8822785 |
| 1224 | 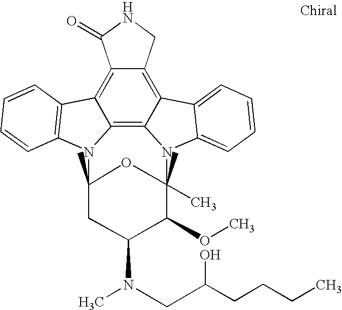 | Novar- tis | | EP 296110 |
| 1225 | 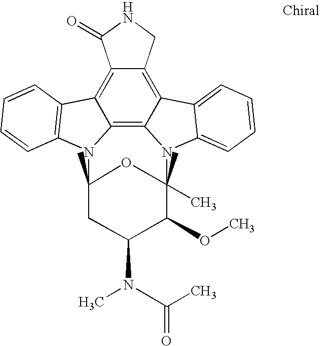 | Novar- tis | | EP 296110 |
| 1226 | 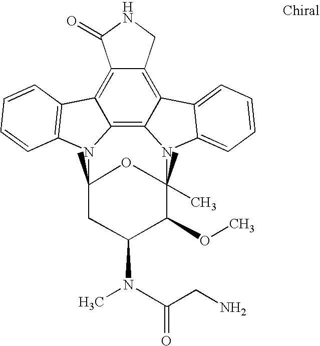 | Novar- tis | | EP 296110 |
| 1227 | 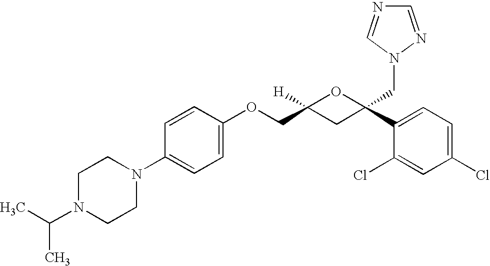 | Scher- ing- Plough | | EP 318214 |
| 1228 | 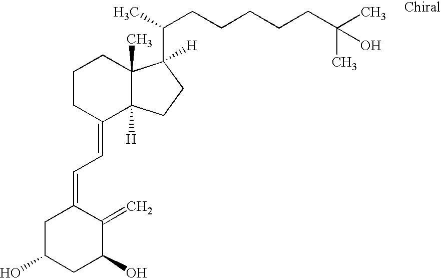 | Leo | | WO 8910351 |
| 1229 | 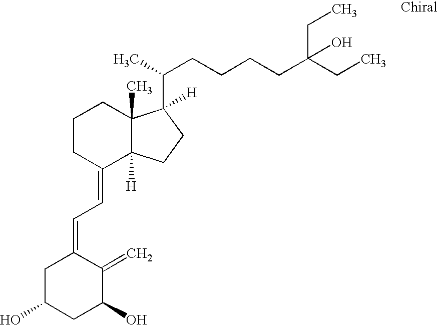 | Leo | | WO 8910351 |
| 1230 | 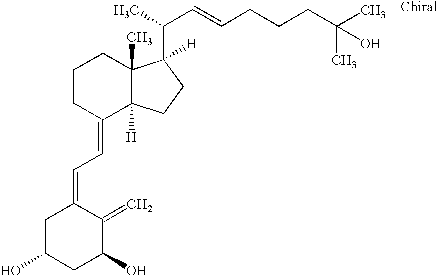 | Leo | | WO 8910351 |
| 1231 | 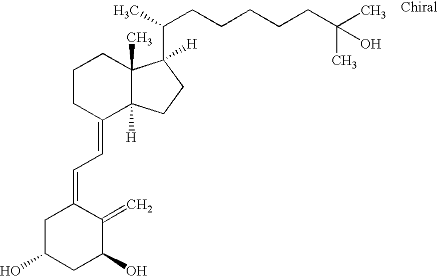 | Leo | | WO 8910351 |
| 1232 | 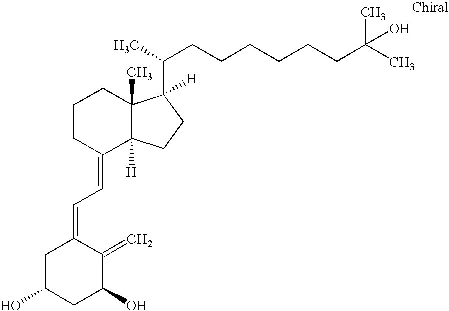 | Leo | | WO 8910351 |
| 1233 | 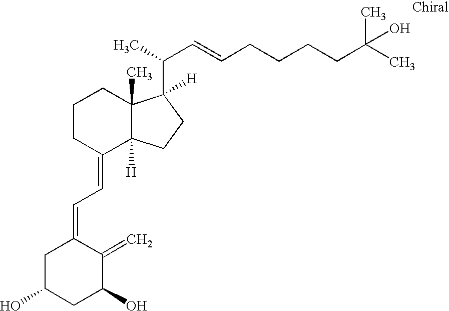 | Leo | | WO 8910351 |
| 1234 | 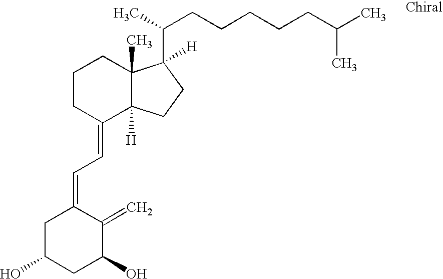 | Leo | | WO 8910351 |
| 1235 | 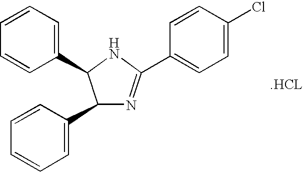 | Tanabe Seiyaku | 1) Ueno, M. et al. Jpn J Pharmacol 1992, 58 (Suppl. 1): Abst O-210. | AU 8942368 |
| 1236 | 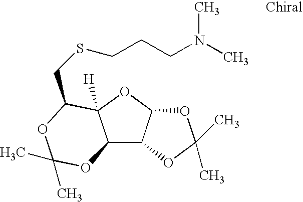 | Green- wich Pharm. | | AU 9047648 |
| 1237 | 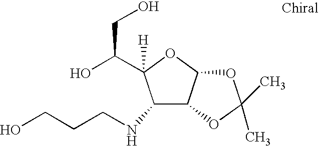 | Green- wich Pharm. | | AU 9047648 |
| 1238 | 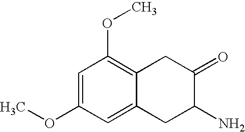 | Aventis Pharma | | EP 378456 |
| 1239 | 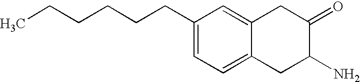 | Aventis Pharma | | EP 378456 |
| 1240 | 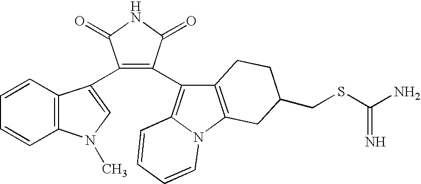 | Roche | | EP 384349 |
| 1241 | 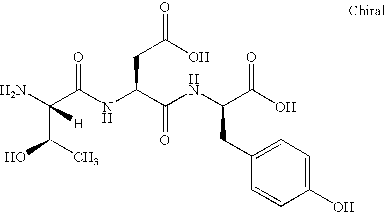 | SPA | | EP 421074 |
| 1242 | 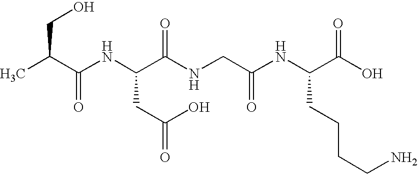 | Hitachi Chemi- cal | | EP 421682 |
| 1243 | 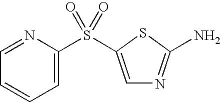 | Fuji- sawa | 1) Manda, T. et al. Jpn J Pharmacol 1997, 73 (Suppl. 1): Abst P-140. | EP 412404 |
| 1244 | 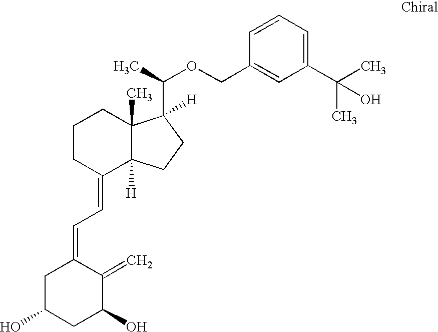 | Leo | | WO 9109841 |
| 1245 | 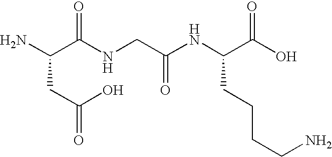 | Hitachi Chemi- cal | | EP 421682 |
| 1246 | 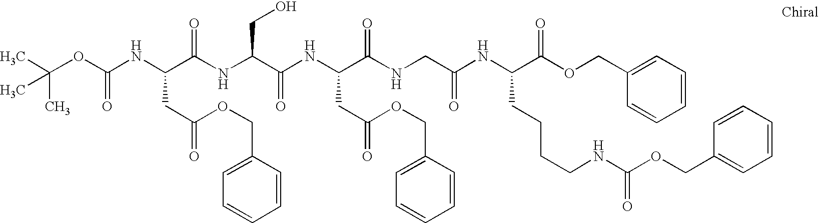 | Hitachi Chemi- cal | | EP 421682 |
| 1247 | 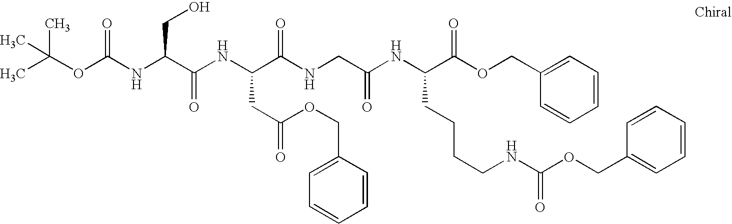 | Hitachi Chemi- cal | | EP 421682 |
| 1248 | 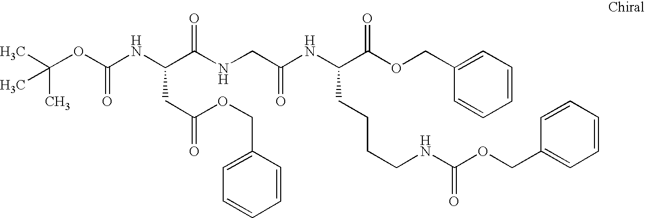 | Hitachi Chemi- cal | | EP 421682 |
| 1249 | 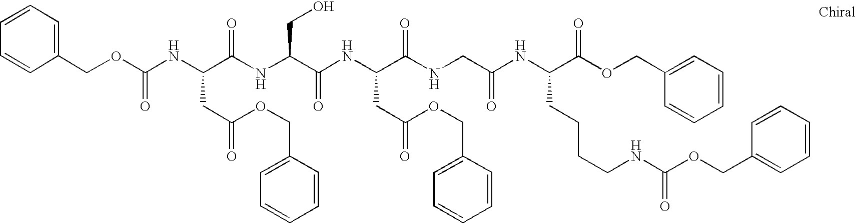 | Hitachi Chemi- cal | | EP 421682 |
| 1250 | 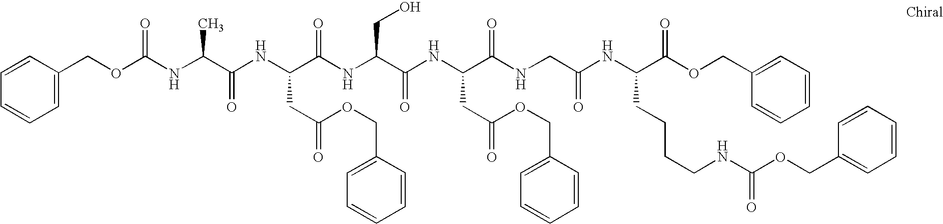 | Hitachi Chemi- cal | | EP 421682 |
| 1251 | 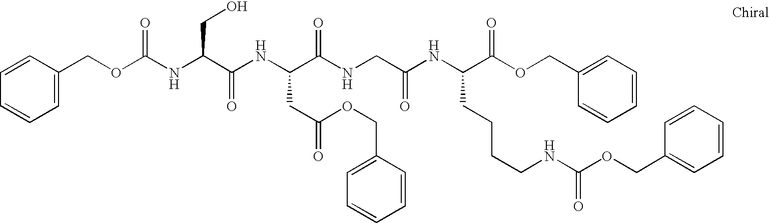 | Hitachi Chemi- cal | | EP 421682 |
| 1252 | 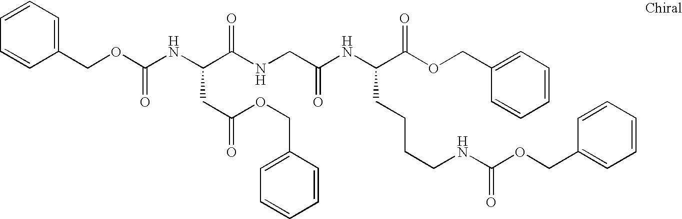 | Hitachi Chemi- cal | | EP 421682 |
| 1253 | 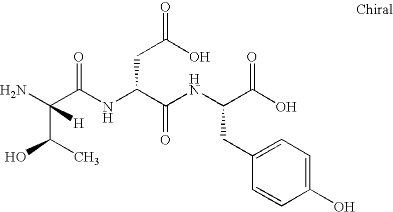 | SPA | | EP 421074 |
| 1254 | 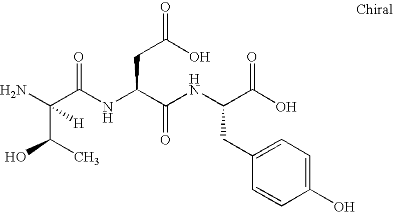 | SPA | | EP 421074 |
| 1255 | 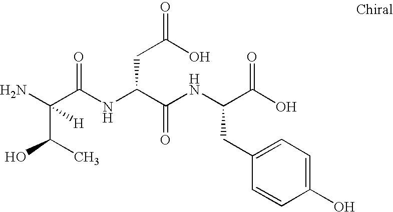 | SPA | | EP 421074 |
| 1256 | 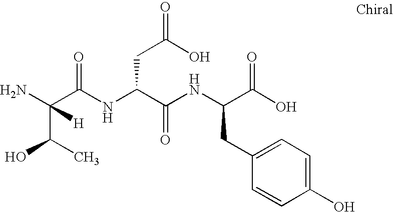 | SPA | | EP 421074 |
| 1257 | 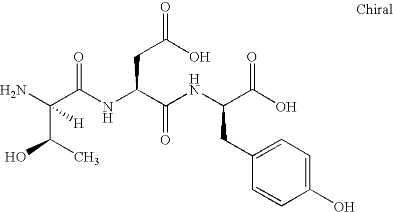 | SPA | | EP 421074 |
| 1258 | 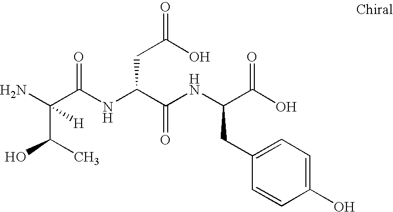 | SPA | | EP 421074 |
| 1259 | 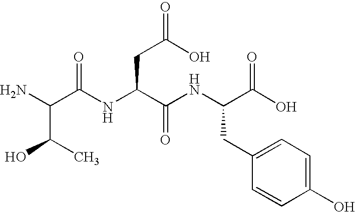 | SPA | | EP 421074 |
| 1260 | 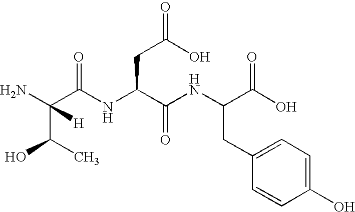 | SPA | | EP 421074 |
| 1261 | 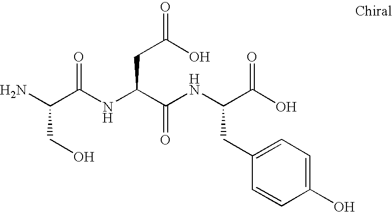 | SPA | | EP 421074 |
| 1262 | 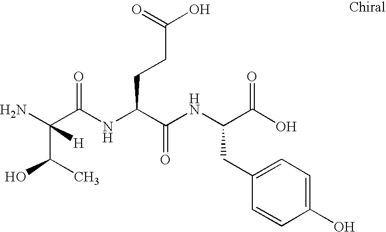 | SPA | | EP 421074 |
| 1263 | 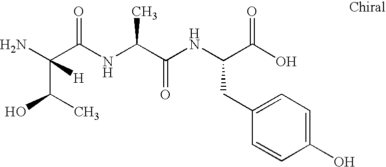 | SPA | | EP 421074 |
| 1264 | 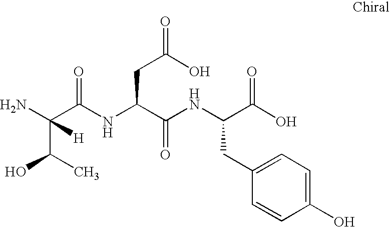 | SPA | | EP 421074 |
| 1265 | 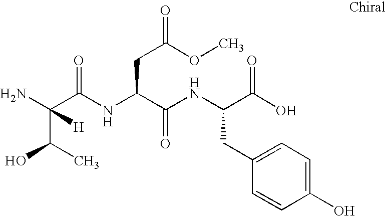 | SPA | | EP 421074 |
| 1266 | 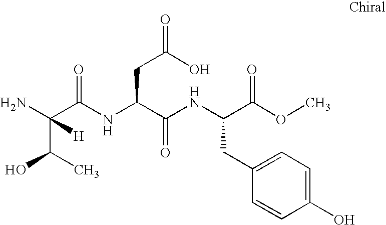 | SPA | | EP 421074 |
| 1267 | 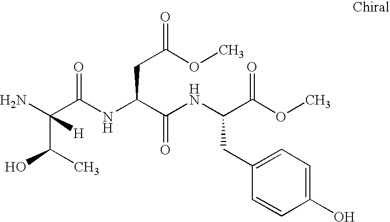 | SPA | | EP 421074 |
| 1268 | 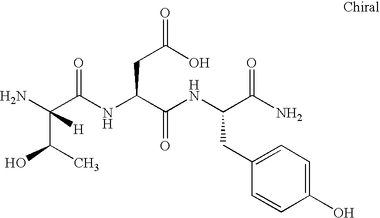 | SPA | | EP 421074 |
| 1269 | 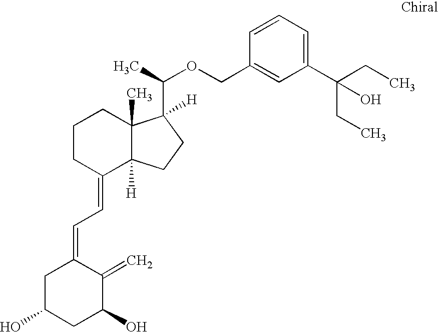 | Leo | | EP 506794 |
| 1270 | 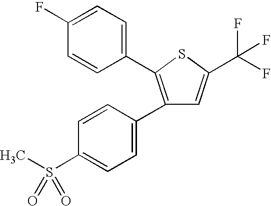 | Fiji- sawa | | WO 9119708 |
| 1271 | 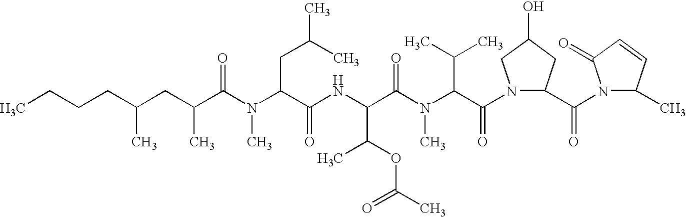 | Harbor Branch Found. | | U.S. Pat. No. 5091368 |
| 1272 | 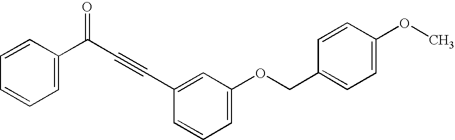 | Aventis Pharma | | EP 476658 |
| 1273 | 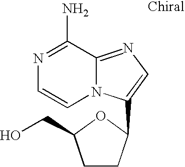 | Merck & Co. | | EP 480713 |
| 1274 | 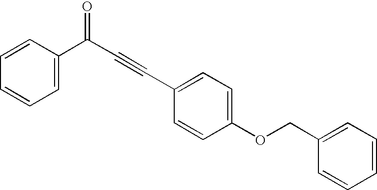 | Aventis Pharma | | EP 476658 |
| 1275 | 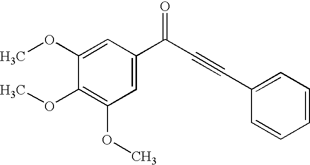 | Aventis Pharma | | EP 476658 |
| 1276 | 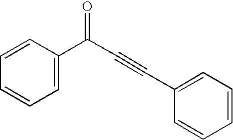 | Aventis Pharma | | EP 476658 |
| 1277 | 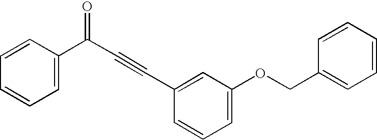 | Aventis Pharma | | EP 476658 |
| 1278 | 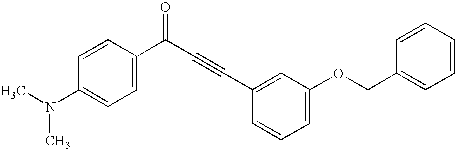 | Aventis Pharma | | EP 476658 |
| 1279 | 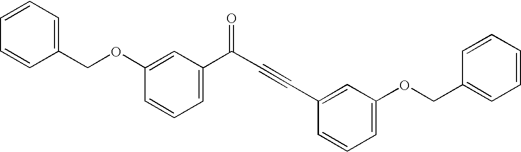 | Aventis Pharma | | EP 476658 |
| 1280 | 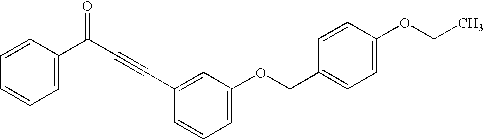 | Aventis Pharma | | EP 476658 |
| 1281 | 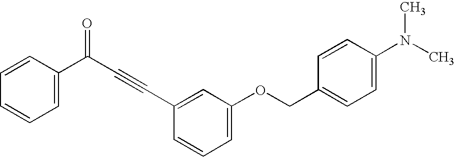 | Aventis Pharma | | EP 476658 |
| 1282 | 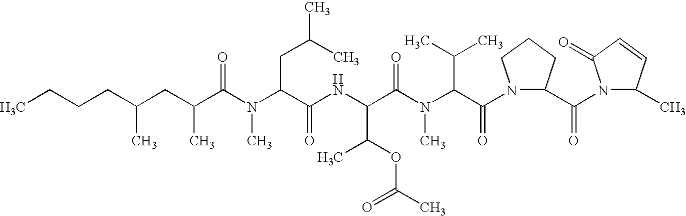 | Harbor Branch Found. | | U.S. Pat. No. 5091368 |
| 1283 | 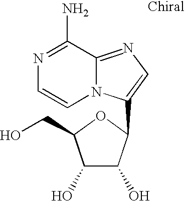 | Merck & Co. | | EP 480713 |
| 1284 | 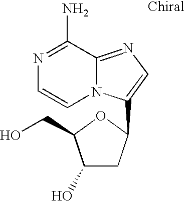 | Merck & Co. | | EP 480713 |
| 1285 | 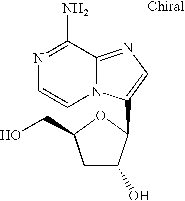 | Merck & Co. | | EP 480713 |
| 1286 | 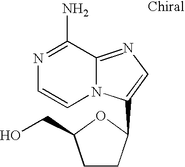 | Merck & Co. | | EP 480713 |
| 1287 | 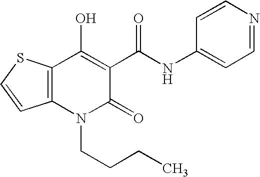 | Kyowa Hakko | Miwa, K. et al. 113th Annu Meet Pharmaceut Soc Jpn (March 29-31, Osaka) 1993, Abst 30CC 13-1. | EP 505058 |
| 1288 | 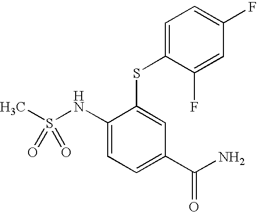 | Fuji- sawa | Nakamura, K. et al. Chem Pharm Bull 1993, 41(5): 894. | AU 8783152 |
| 1289 | 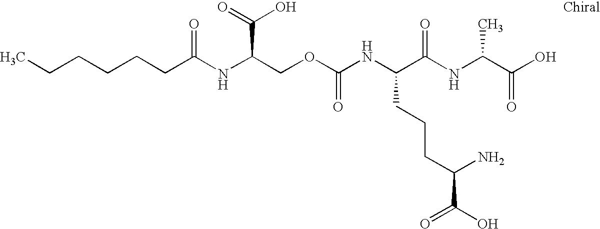 | Wyeth | | U.S. Pat. No. 5312831 |
| 1290 | 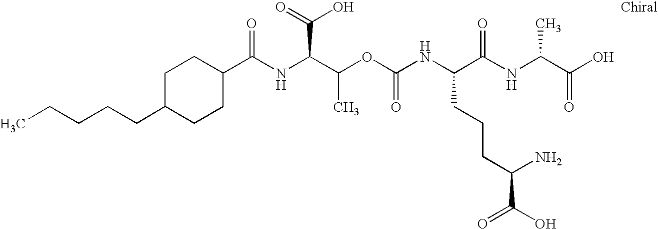 | Wyeth | | U.S. Pat. No. 5312831 |
| 1291 | 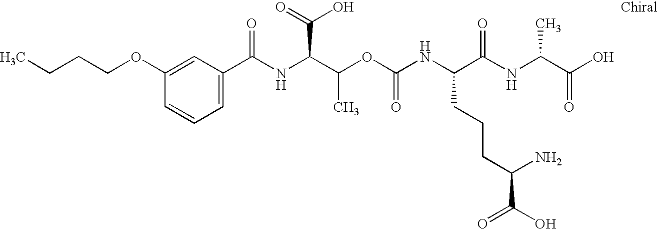 | Wyeth | | U.S. Pat. No. 5312831 |
| 1292 | 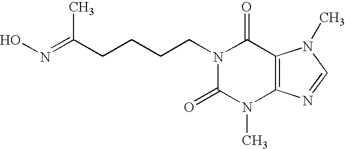 | Cell Thera- peutics | | WO 9416704 |
| 1293 | 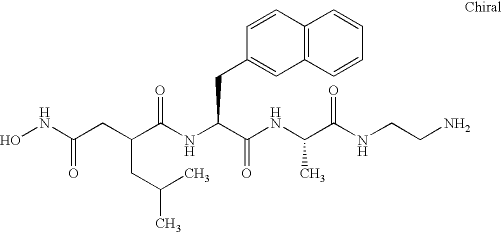 | Immu- nex | 1) Immunex Corporation Press Release 1994, July 21. | WO 9506031 |
| 1294 | 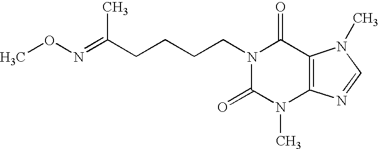 | Cell Thera- peutics | | WO 9416704 |
| 1295 | 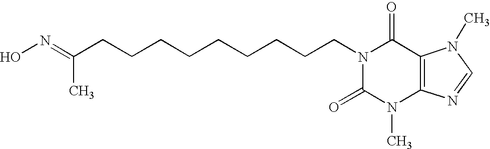 | Cell Thera- peutics | | WO 9416704 |
| 1296 | 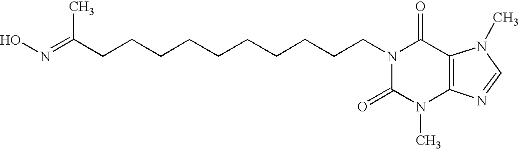 | Cell Thera- peutics | | WO 9416704 |
| 1297 | 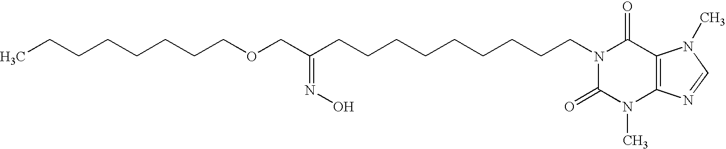 | Cell Thera- peutics | | WO 9416704 |
| 1298 | 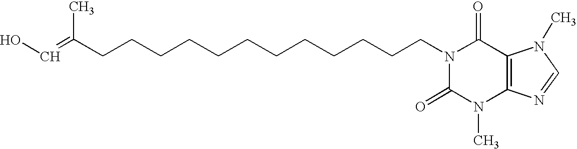 | Cell Thera- peutics | | WO 9416704 |
| 1299 | 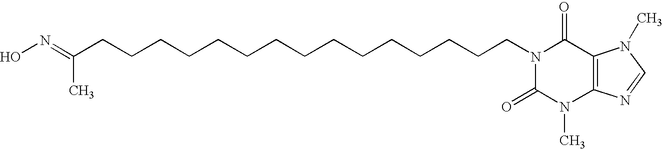 | Cell Thera- peutics | | WO 9416704 |
| 1300 | 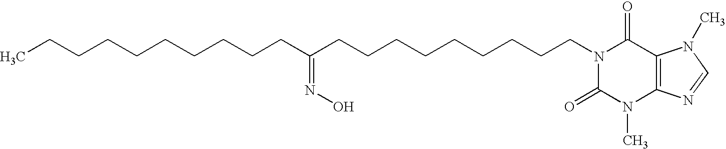 | Cell Thera- peutics | | WO 9416704 |
| 1301 | 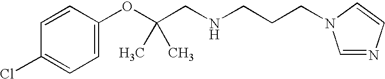 | Abbott GmbH | | WO 9500493 |
| 1302 | 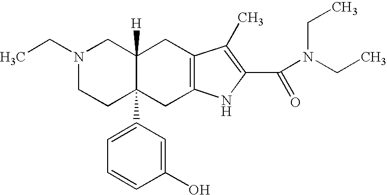 | Glaxo- Smith Kline | | WO 9504734 |
| 1303 | 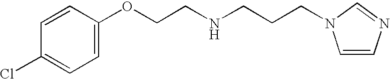 | Abbott GmbH | | WO 9500493 |
| 1304 | 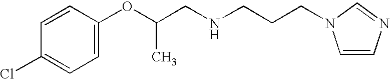 | Abbott GmbH | | WO 9500493 |
| 1305 | 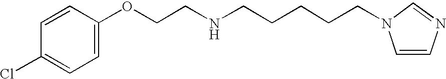 | Abbott GmbH | | WO 9500493 |
| 1306 | 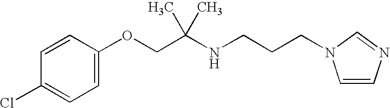 | Abbott GmbH | | WO 9500493 |
| 1307 | 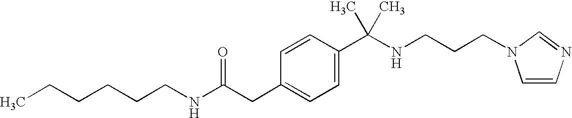 | Abbott GmbH | | WO 9500493 |
| 1308 | 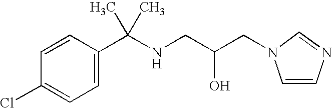 | Abbott GmbH | | WO 9500493 |
| 1309 | 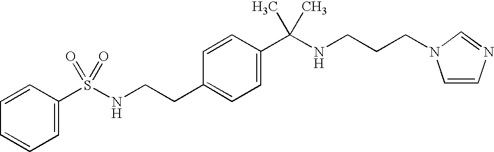 | Abbott GmbH | | WO 9500493 |
| 1310 | 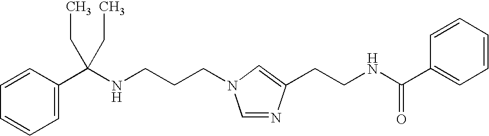 | Abbott GmbH | | WO 9500493 |
| 1311 | 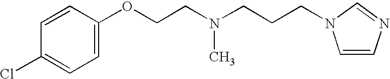 | Abbott GmbH | | WO 9500493 |
| 1312 | 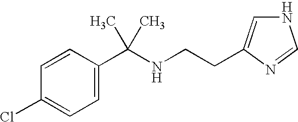 | Abbott GmbH | | WO 9500493 |
| 1313 | 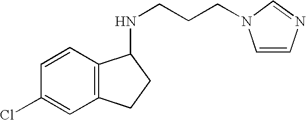 | Abbott GmbH | | WO 9500493 |
| 1314 | 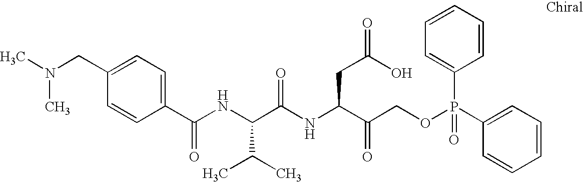 | Sanofi- Synthe- labo | | EP 644197 |
| 1315 | 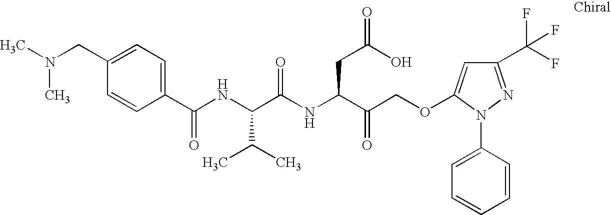 | Sanofi- Synthe- labo | | CA 2125021 |
| 1316 | 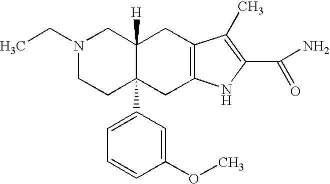 | Glaxo Smith Kline | | WO 9504734 |
| 1317 | 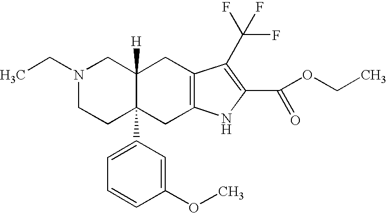 | Glaxo Smith Kline | | WO 9504734 |
| 1318 | 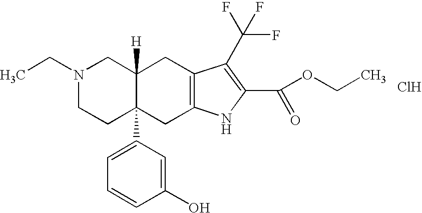 | Glaxo Smith Kline | | WO 9504734 |
| 1319 | 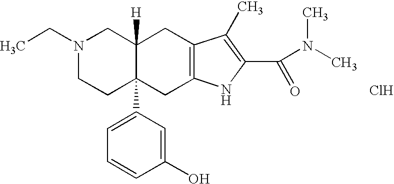 | Glaxo Smith Kline | | WO 9504734 |
| 1320 | 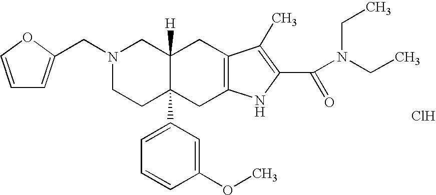 | Glaxo Smith Kline | | WO 9504734 |
| 1321 | 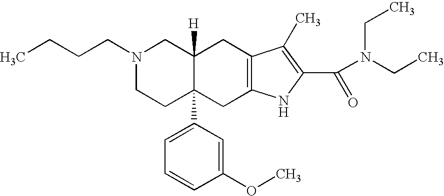 | Glaxo Smith Kline | | WO 9504734 |
| 1322 | 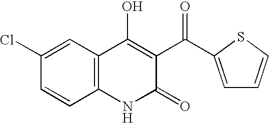 | Japan Tobac- co | | JP 95002779 |
| 1323 | 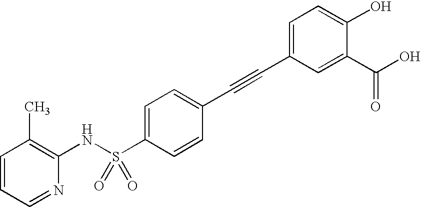 | Pharma- cia | 1) Gozzi, P. et al. J Pharmacol Exp Ther 1999, 291(1): 199. | JP 1995501330 |
| 1324 | 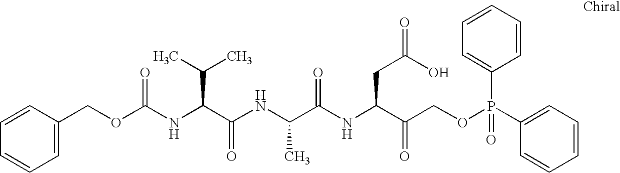 | Sanofi- Synthe- labo | | EP 644197 |
| 1325 | 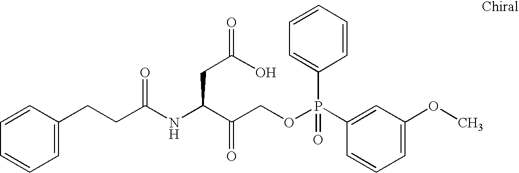 | Sanofi- Synthe- labo | | EP 644197 |
| 1326 | 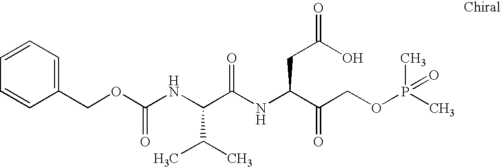 | Sanofi- Synthe- labo | | EP 644197 |
| 1327 | 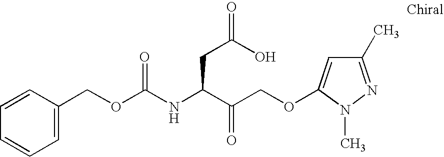 | Sanofi- Synthe- labo | | CA 212021 |
| 1328 | 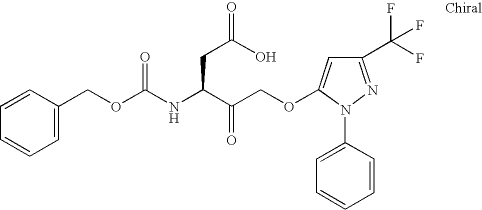 | Sanofi- Synthe- labo | | CA 212021 |
| 1329 | 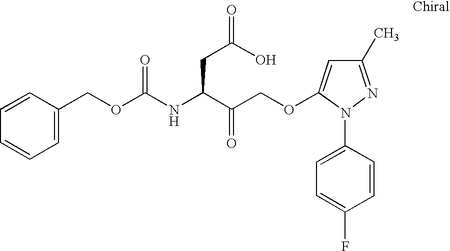 | Sanofi- Synthe- labo | | CA 212021 |
| 1330 | 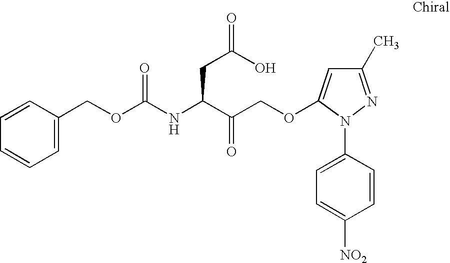 | Sanofi- Synthe- labo | | CA 212021 |
| 1331 | 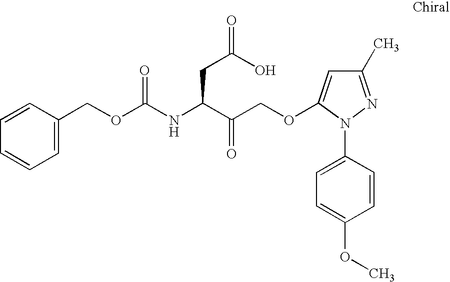 | Sanofi- Synthe- labo | | CA 212021 |
| 1332 | 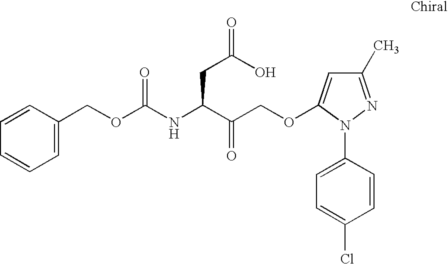 | Sanofi- Synthe- labo | | CA 212021 |
| 1333 | 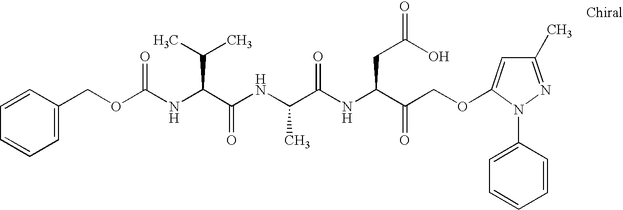 | Sanofi- Synthe- labo | | CA 212021 |
| 1334 | 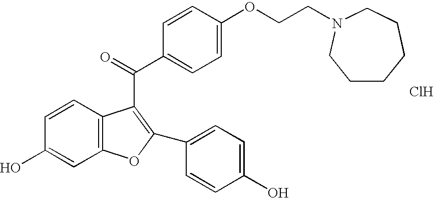 | Lilly | | WO 9517382 |
| 1335 | 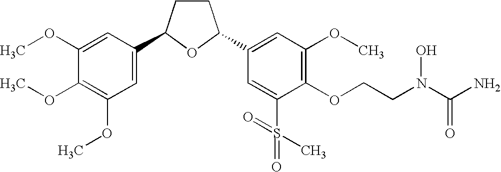 | Millen- nium | | WO 9518610 |
| 1336 | 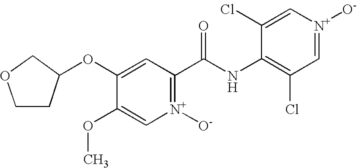 | Aventis Pharma | | WO 9520578 |
| 1337 | 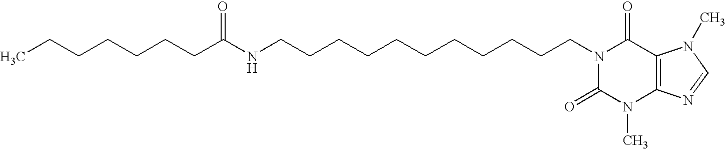 | Cell Thera- peutics | | WO 9522546 |
| 1338 | 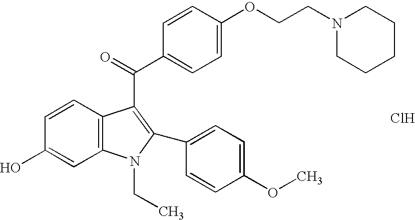 | Lilly | | WO 9517382 |
| 1339 | 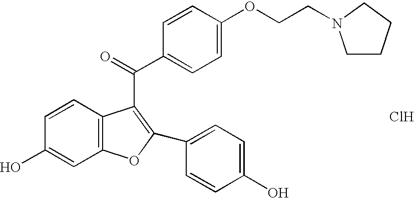 | Lilly | | WO 9517382 |
| 1340 | 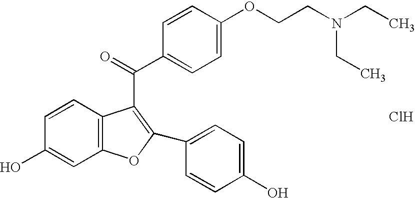 | Lilly | | WO 9517382 |
| 1341 | 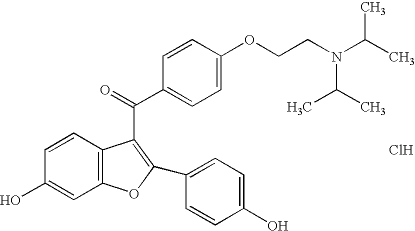 | Lilly | | WO 9517382 |
| 1342 | 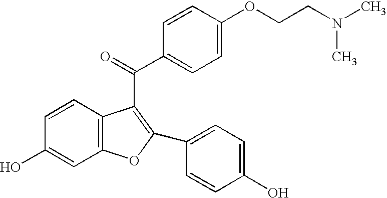 | Lilly | | WO 9517382 |
| 1343 | 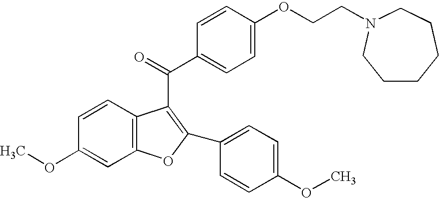 | Lilly | | WO 9517382 |
| 1344 | 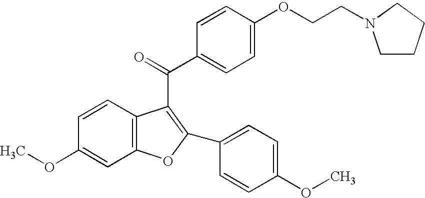 | Lilly | | WO 9517382 |
| 1345 | 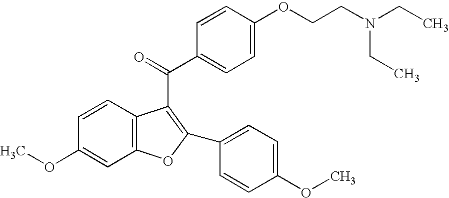 | Lilly | | WO 9517382 |
| 1346 | 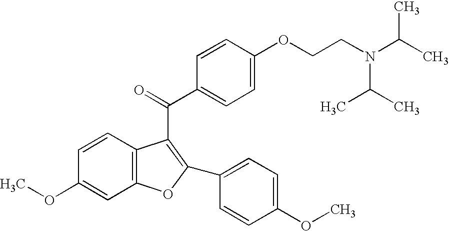 | Lilly | | WO 9517382 |
| 1347 | 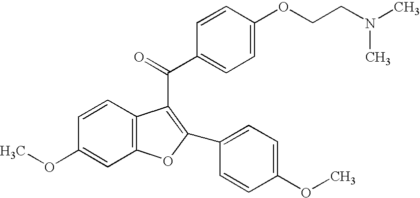 | Lilly | | WO 9517382 |
| 1348 | 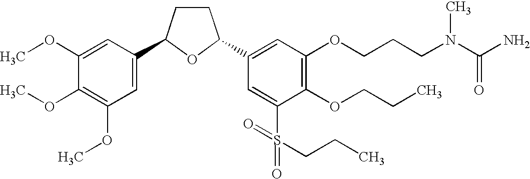 | Cyto Med | | WO 9518610 |
| 1349 | 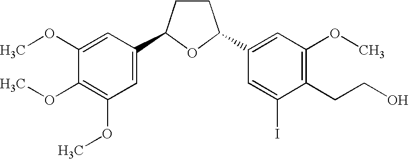 | Cyto Med | | WO 9518610 |
| 1350 | 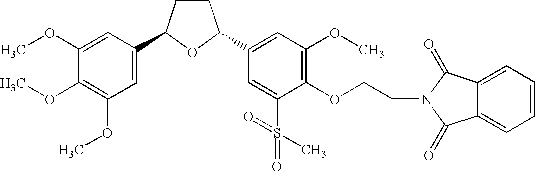 | Cyto Med | | WO 9518610 |
| 1351 | 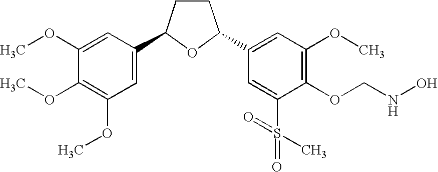 | Cyto Med | | WO 9518610 |
| 1352 | 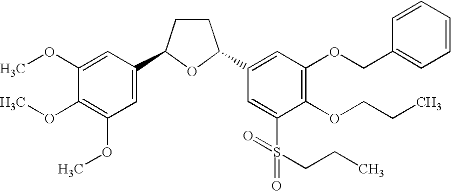 | Cyto Med | | WO 9518610 |
| 1353 | 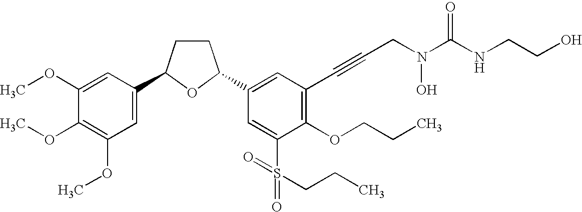 | Cyto Med | | WO 9518610 |
| 1354 | 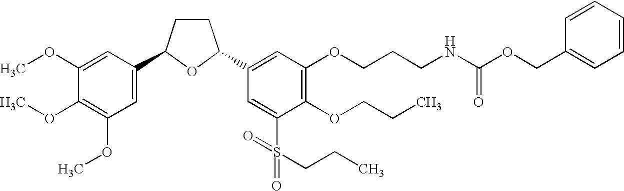 | Cyto Med | | WO 9518610 |
| 1355 | 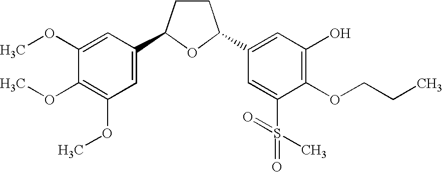 | Cyto Med | | WO 9518610 |
| 1356 | 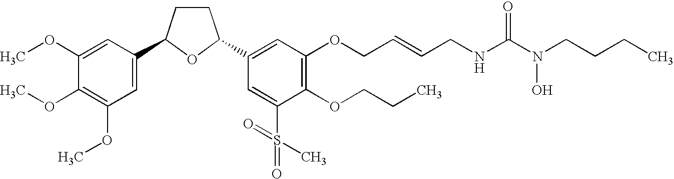 | Cyto Med | | WO 9518610 |
| 1357 | 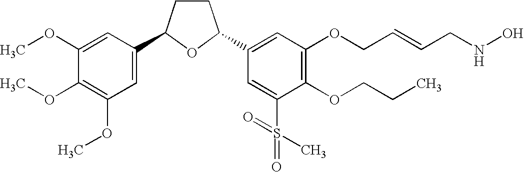 | Cyto Med | | WO 9518610 |
| 1358 | 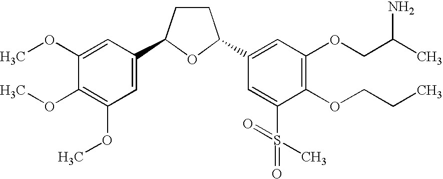 | Cyto Med | | WO 9518610 |
| 1359 | 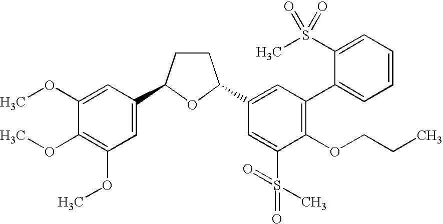 | Cyto Med | | WO 9518610 |
| 1360 | 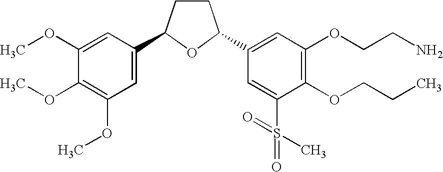 | Cyto Med | | WO 9518610 |
| 1361 | 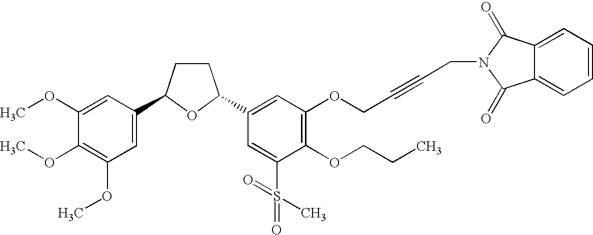 | Cyto Med | | WO 9518610 |
| 1362 | 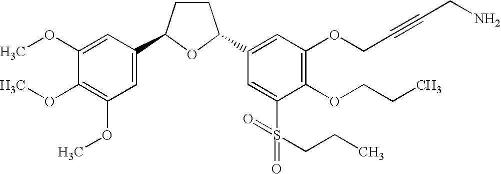 | Cyto Med | | WO 9518610 |
| 1363 | 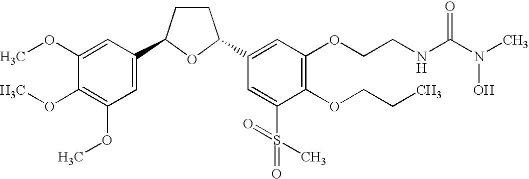 | Cyto Med | | WO 9518610 |
| 1364 | 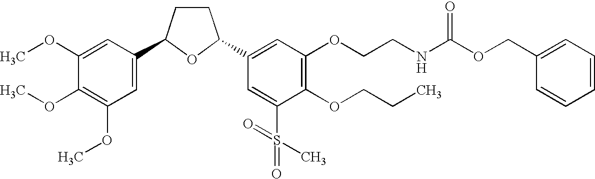 | Cyto Med | | WO 9518610 |
| 1365 | 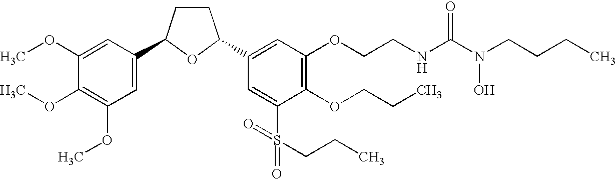 | Cyto Med | | WO 9518610 |
| 1366 | 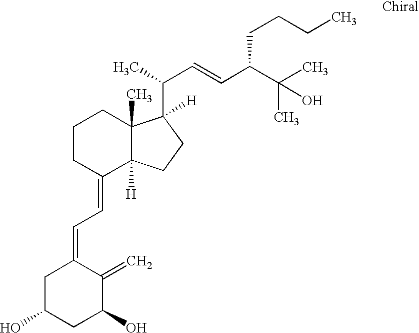 | Duphar | | EP 664287 |
| 1367 | 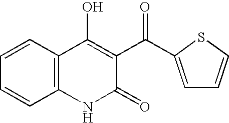 | Japan Tobac- co | | JP 95002779 |
| 1368 | 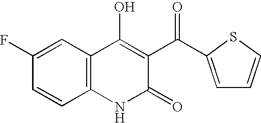 | Japan Tobac- co | | JP 95002779 |
| 1369 | 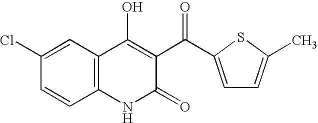 | Japan Tobac- co | | JP 95002779 |
| 1370 | 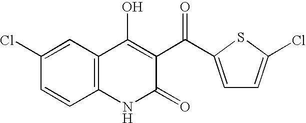 | Japan Tobac- co | | JP 95002779 |
| 1371 | 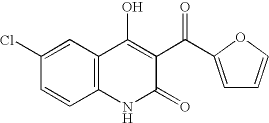 | Japan Tobac- co | | JP 95002779 |
| 1372 | 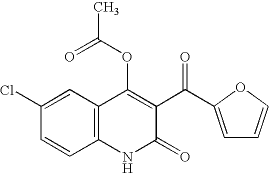 | Japan Tobac- co | | JP 95002779 |
| 1373 | 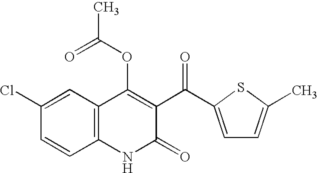 | Japan Tobac- co | | JP 95002779 |
| 1374 | 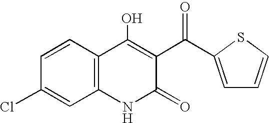 | Japan Tobac- co | | JP 95002779 |
| 1375 | 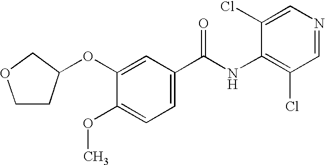 | Aventis Pharma | | WO 9520578 |
| 1376 | 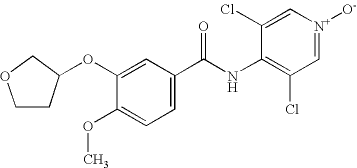 | Aventis Pharma | | WO 9520578 |
| 1377 | 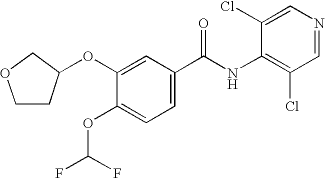 | Aventis Pharma | | WO 9520578 |
| 1378 | 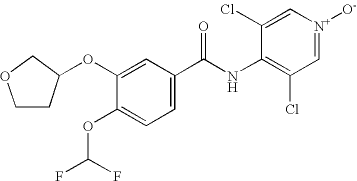 | Aventis Pharma | | WO 9520578 |
| 1379 | 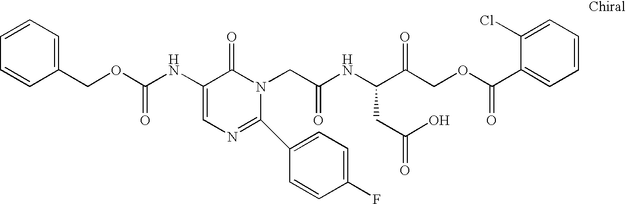 | Sanofi- Synthe- labo | | WO 9526958 |
| 1380 | 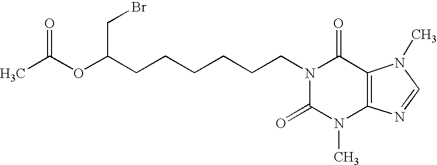 | Cell Thera- peutics | | WO 9522546 |
| 1381 | 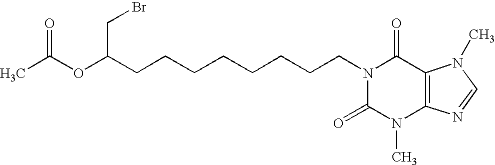 | Cell Thera- peutics | | WO 9522546 |
| 1382 | 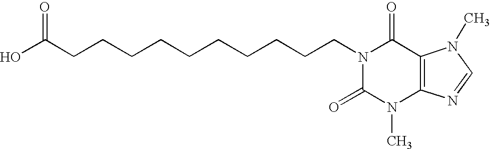 | Cell Thera- peutics | | WO 9522546 |
| 1383 | 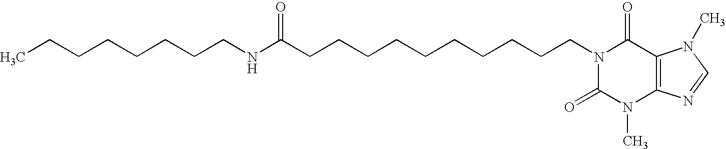 | Cell Thera- peutics | | WO 9522546 |
| 1384 | 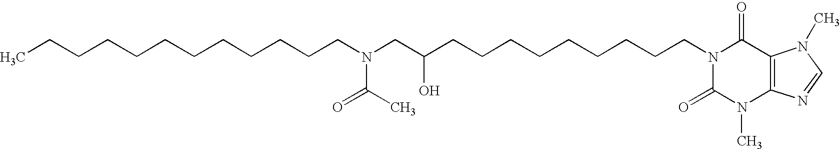 | Cell Thera- peutics | | WO 9522546 |
| 1385 | 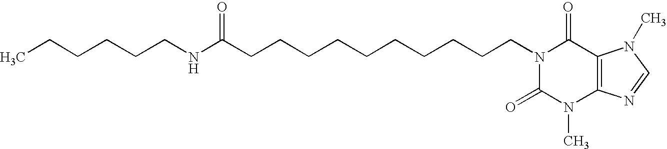 | Cell Thera- peutics | | WO 9522546 |
| 1386 | 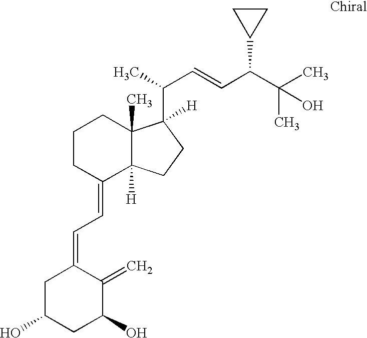 | Duphar | | EP 664287 |
| 1387 | 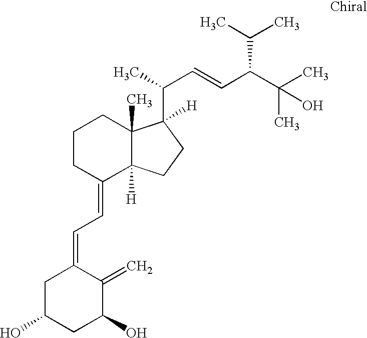 | Duphar | | EP 664287 |
| 1388 | 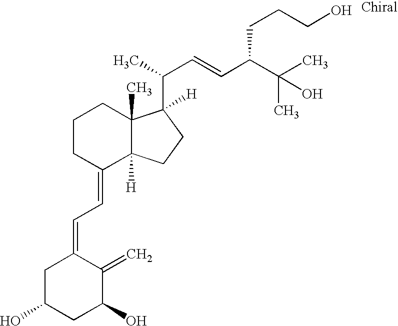 | Duphar | | EP 664287 |
| 1389 | 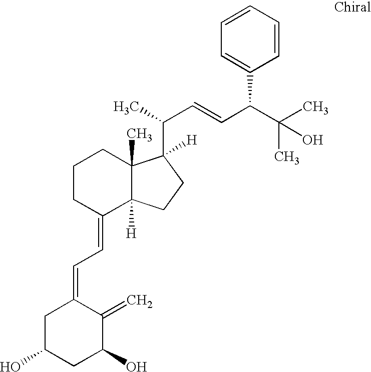 | Duphar | | EP 664287 |
| 1390 | 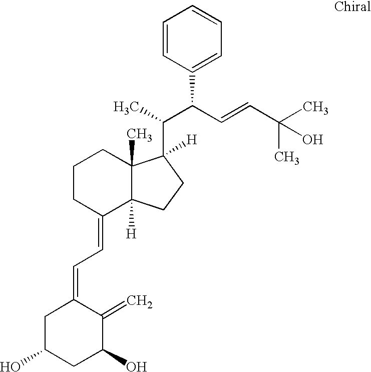 | Duphar | | EP 664287 |
| 1391 | 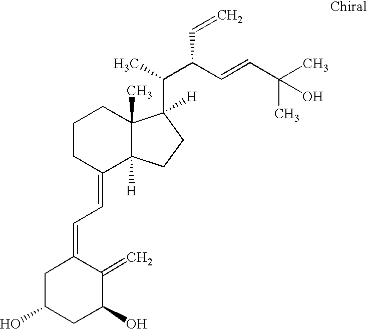 | Duphar | | EP 664287 |
| 1392 | 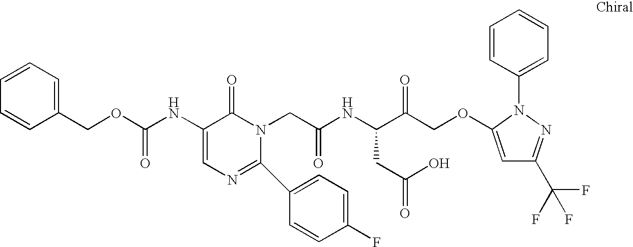 | Sanofi- Synthe- labo | | WO 9526958 |
| 1393 | 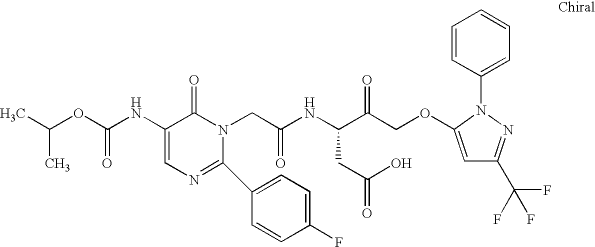 | Sanofi- Synthe- labo | | WO 9526958 |
| 1394 | 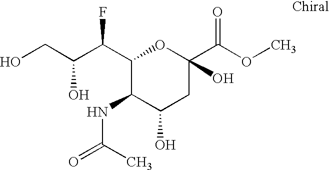 | Daikin | | EP 711766 |
| 1395 | 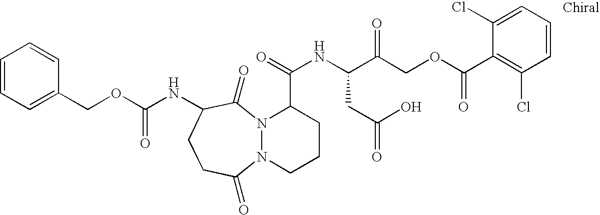 | Sanofi- Synthe- labo | | WO 9533751 |
| 1396 | 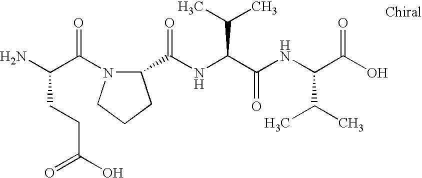 | Ajino- moto | | U.S. Pat. No. 5464918 |
| 1397 | 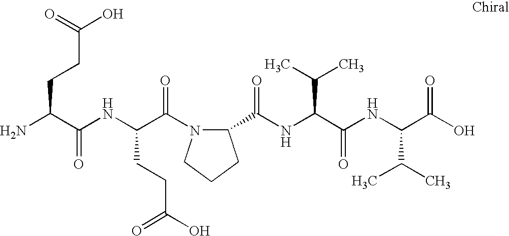 | Ajino- moto | | U.S. Pat. No. 5464918 |
| 1398 | 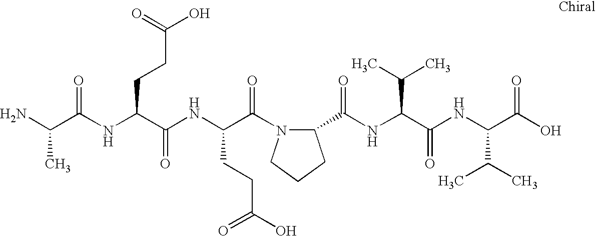 | Ajino- moto | | U.S. Pat. No. 5464918 |
| 1399 | 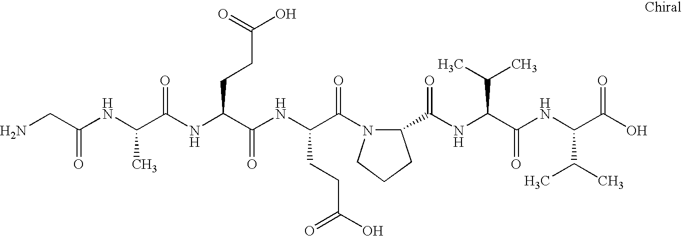 | Ajino- moto | | U.S. Pat. No. 5464918 |
| 1400 | 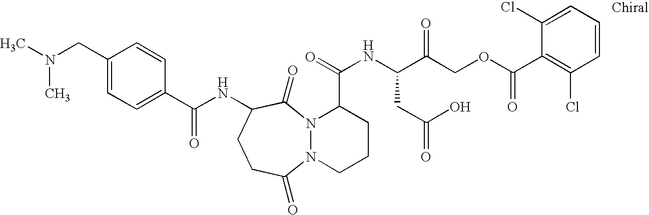 | Sanofi- Synthe- labo | | WO 9533751 |
| 1401 | 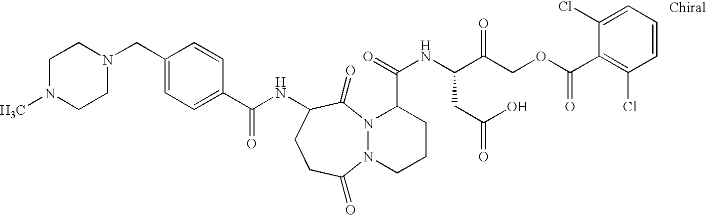 | Sanofi- Synthe- labo | | WO 9533751 |
| 1402 | 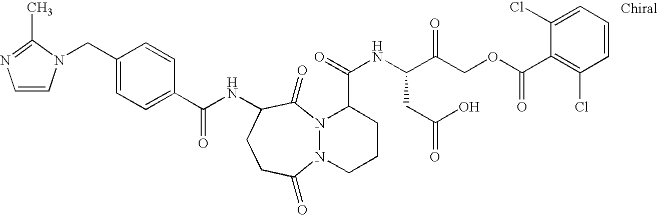 | Sanofi- Synthe- labo | | WO 9533751 |
| 1403 | 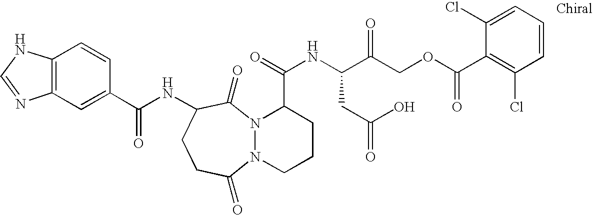 | Sanofi- Synthe- labo | | WO 9533751 |
| 1404 | 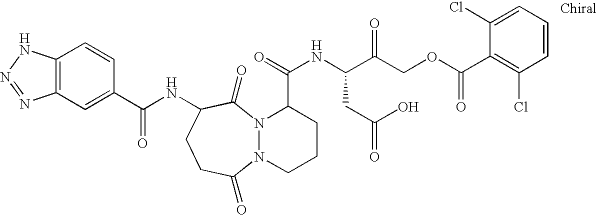 | Sanofi- Synthe- labo | | WO 9533751 |
| 1405 | 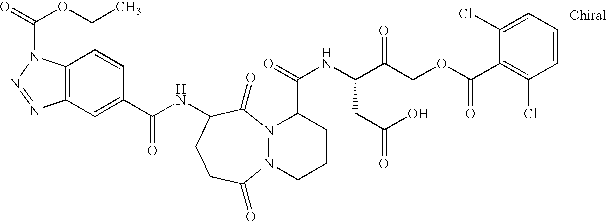 | Sanofi- Synthe- labo | | WO 9533751 |
| 1406 | 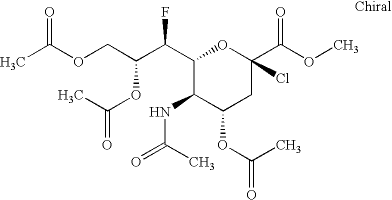 | Daikin | | EP 711766 |
| 1407 | 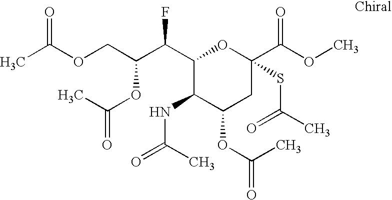 | Daikin | | EP 711766 |
| 1408 | 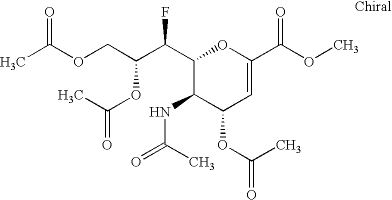 | Daikin | | EP 711766 |
| 1409 | 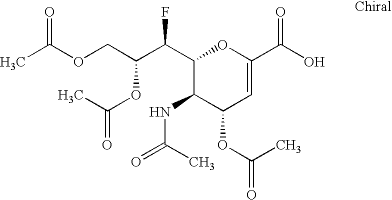 | Daikin | | EP 711766 |
| 1410 | 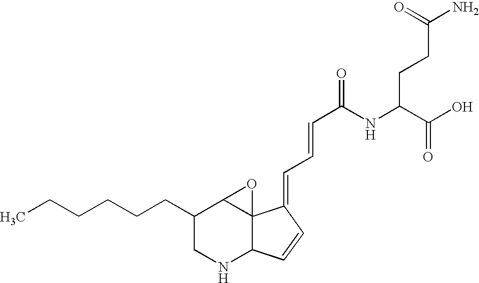 | Micro- bial Chem- istry Re- search Founda- tion | | JP 96176157 |
| 1411 | 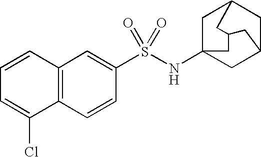 | Tanabe | | WO 9640641 |
| 1412 | 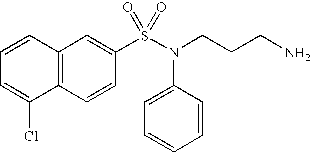 | Tanabe | | WO 9640641 |
| 1413 | 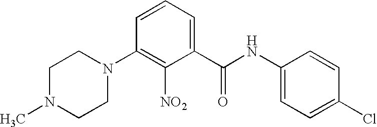 | Daiichi Pharma- ceutical | 1) Kawagoe, K. et al. AFMC Int Med Chem Symp (Sept 3-8, Tokyo) 1995, Abst P13M183. | JP 97059236 |
| 1414 | 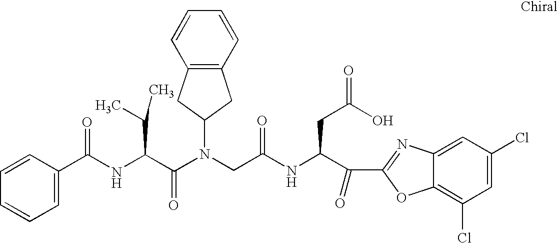 | Vertex | | WO 9722618 |
| 1415 | 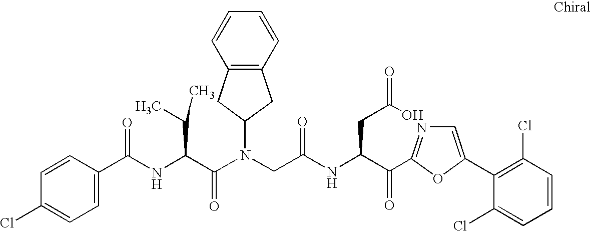 | Vertex | | WO 9722618 |
| 1416 | 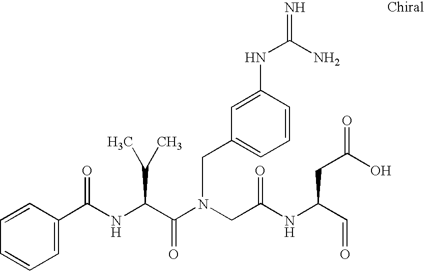 | Vertex | | WO 9722618 |
| 1417 | 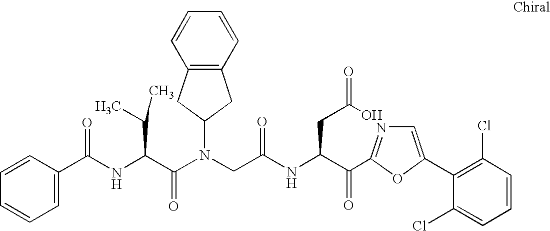 | Vertex | | WO 9722618 |
| 1418 | 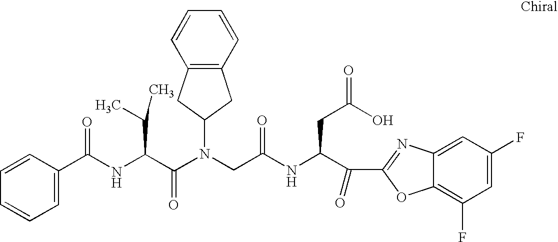 | Vertex | | WO 9722618 |
| 1419 | 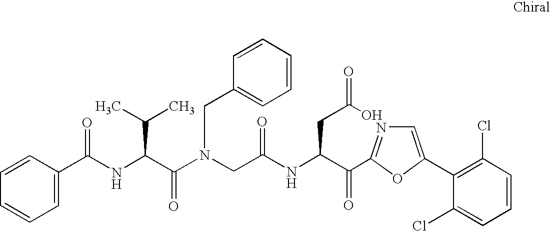 | Vertex | | WO 9722618 |
| 1420 | 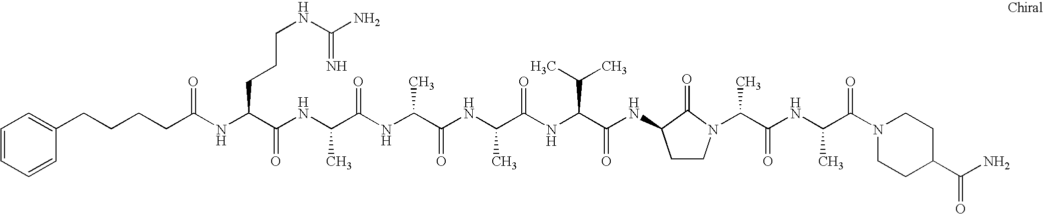 | Astra Zeneca | | WO 9731023 |
| 1421 | 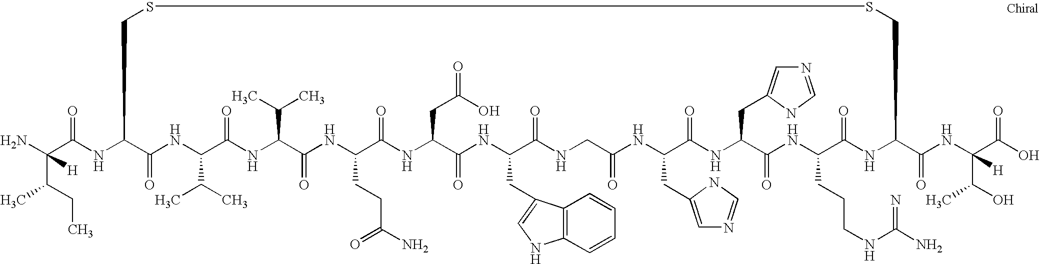 | Univer- sity of Penn- sylvania | | WO 9733603 |
| 1422 | 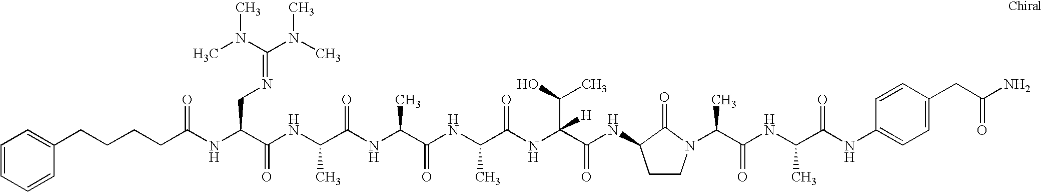 | Astra Zeneca | | WO 9731023 |
| 1423 | 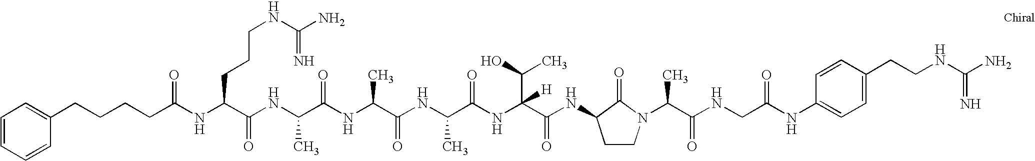 | Astra Zeneca | | WO 9731023 |
| 1424 | 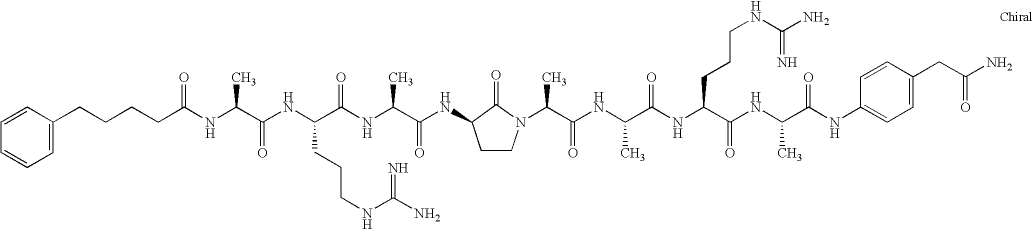 | Astra Zeneca | | WO 9731023 |
| 1425 | 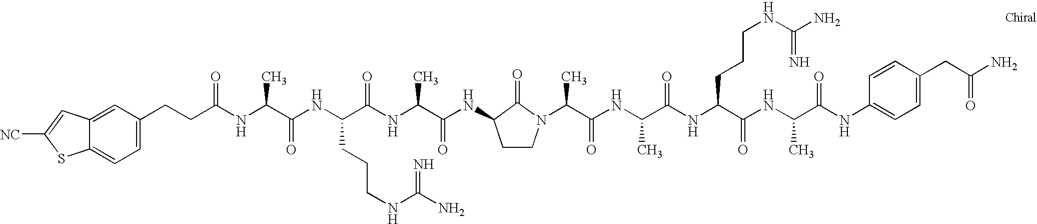 | Astra Zeneca | | WO 9731023 |
| 1426 | 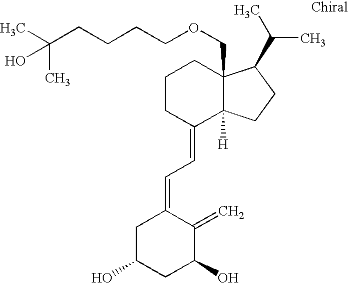 | Leo | | WO 9737972 |
| 1427 | 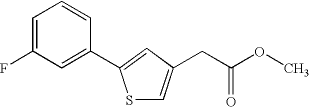 | Taisho | | JP 97194476 |
| 1428 | 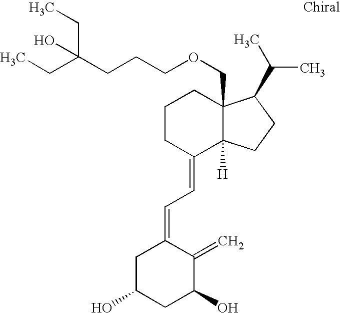 | Leo | | WO 9737972 |
| 1429 | 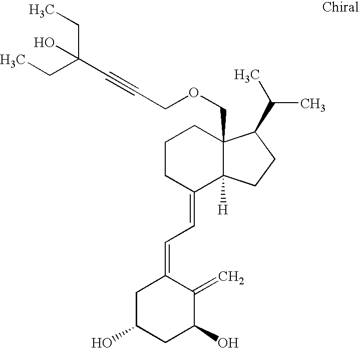 | Leo | | WO 9737972 |
| 1430 | 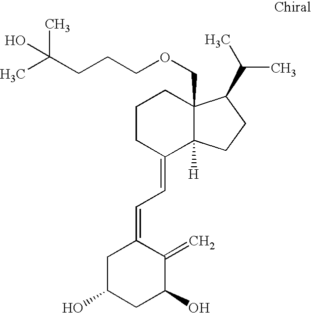 | Leo | | WO 9737972 |
| 1431 | 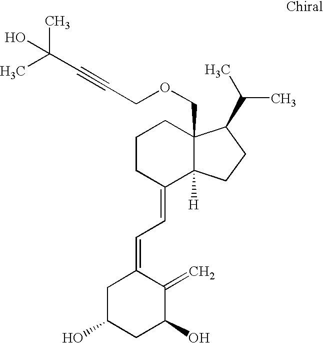 | Leo | | WO 9737972 |
| 1432 | 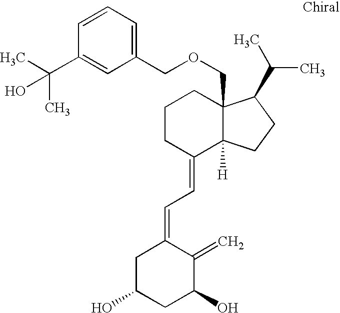 | Leo | | WO 9737972 |
| 1433 | 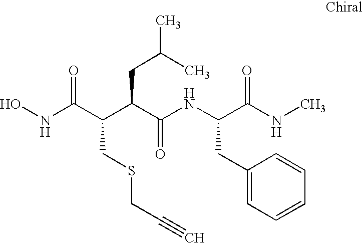 | Glaxo- Smith Kline | | WO 9743250 |
| 1434 | 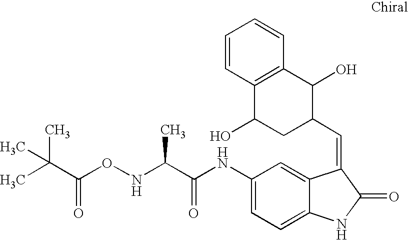 | Pharm- acia | | WO 9745409 |
| 1435 | 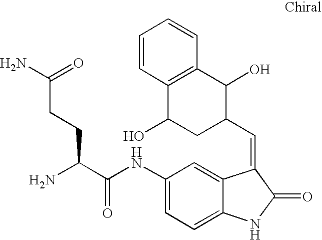 | Pharm- acia | | WO 9745409 |
| 1436 | 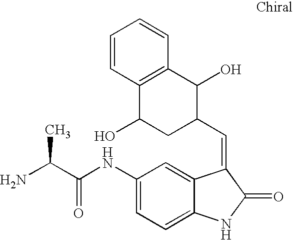 | Pharm- acia | | WO 9745409 |
| 1437 | 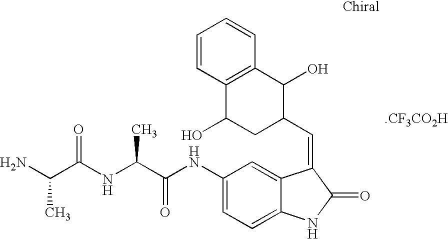 | Pharm- acia | | WO 9745409 |
| 1438 | 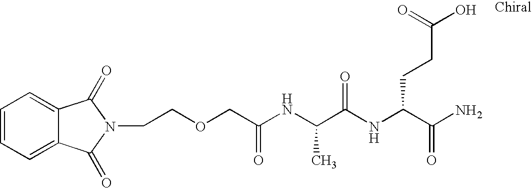 | LEK | | EP 477912 |
| 1439 | 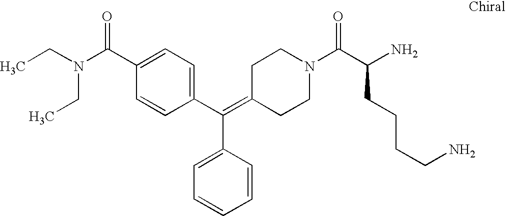 | Astra Zeneca | | WO 9828275 |
| 1440 | 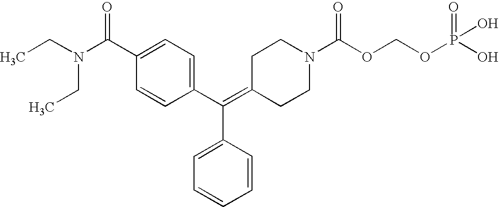 | Astra Zeneca | | WO 9828275 |
| 1441 | 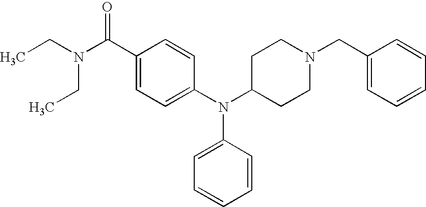 | Astra Zeneca | | WO 9828270 |
| 1442 | 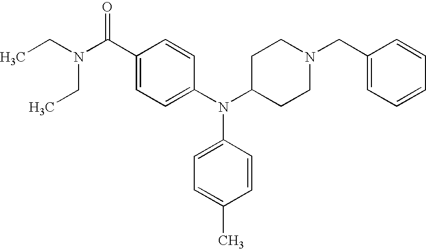 | Astra Zeneca | | WO 9828270 |
| 1443 | 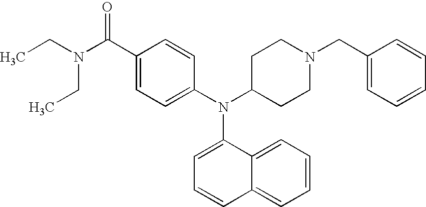 | Astra Zeneca | | WO 9828270 |
| 1444 | 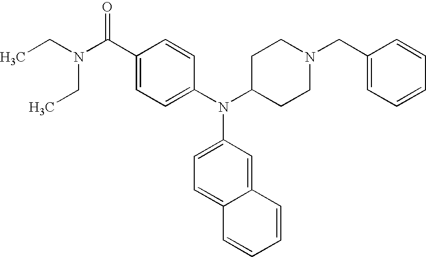 | Astra Zeneca | | WO 9828270 |
| 1445 | 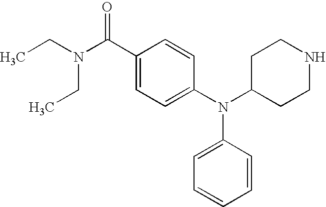 | Astra Zeneca | | WO 9828270 |
| 1446 | 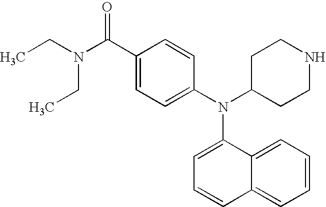 | Astra Zeneca | | WO 9828270 |
| 1447 | 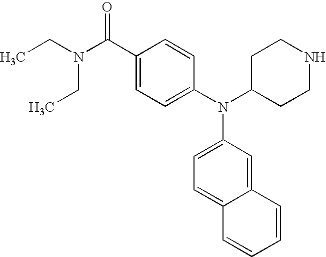 | Astra Zeneca | | WO 9828270 |
| 1448 | 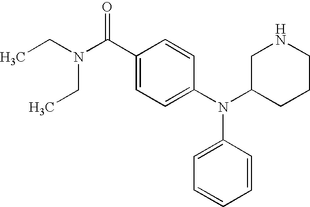 | Astra Zeneca | | WO 9828270 |
| 1449 | 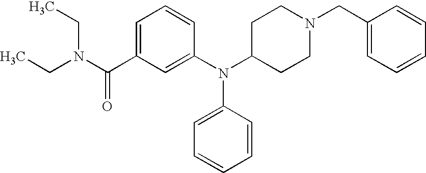 | Astra Zeneca | | WO 9828270 |
| 1450 | 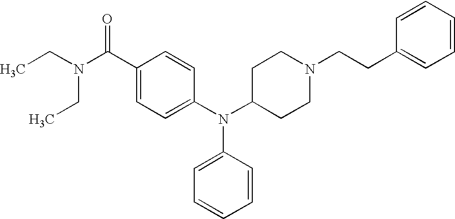 | Astra Zeneca | | WO 9828270 |
| 1451 | 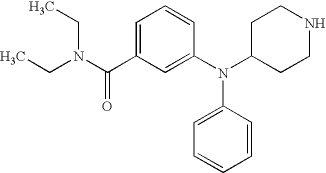 | Astra Zeneca | | WO 9828270 |
| 1452 | 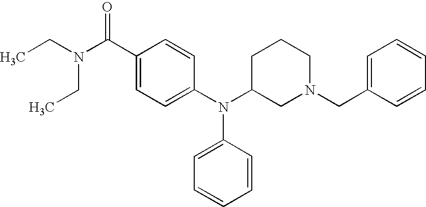 | Astra Zeneca | | WO 9828270 |
| 1453 | 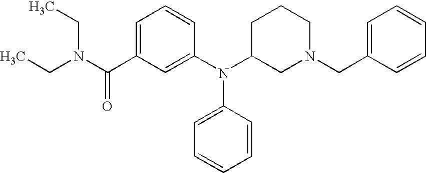 | Astra Zeneca | | WO 9828270 |
| 1454 | 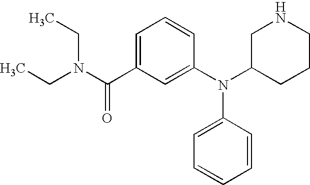 | Astra Zeneca | | WO 9828270 |
| 1455 | 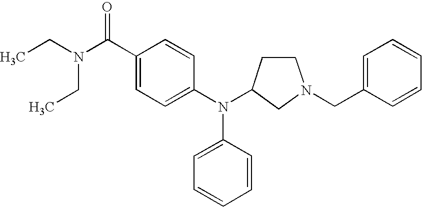 | Astra Zeneca | | WO 9828270 |
| 1456 | 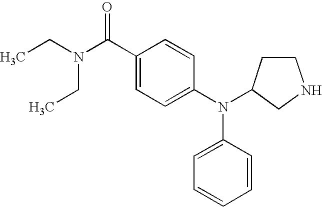 | Astra Zeneca | | WO 9828270 |
| 1457 | 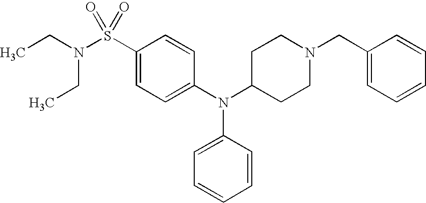 | Astra Zeneca | | WO 9828270 |
| 1458 | 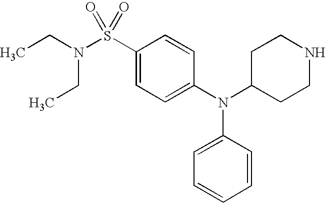 | Astra Zeneca | | WO 9828270 |
| 1459 | 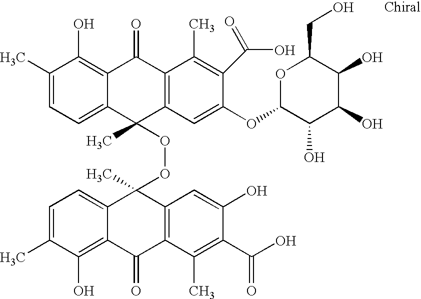 | Daiichi Pharma- ceutical | Koiwa, T. et al. J Antibiot 1999, 52(2): 198. | |
| 1460 | 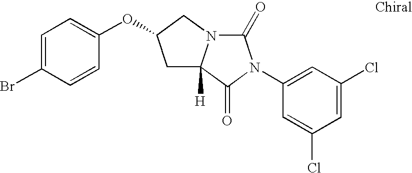 | Bristol- Myers Squibb | | WO 0244181 |
| 1461 | 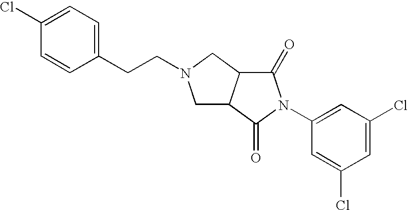 | Bristol- Myers Squibb | | WO 0244181 |
| 1462 | 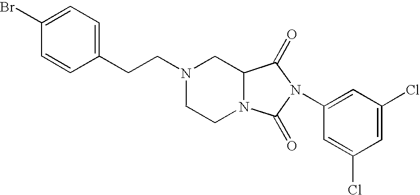 | Bristol- Myers Squibb | | WO 0244181 |
| 1463 | 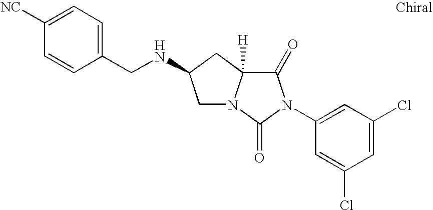 | Bristol- Myers Squibb | | WO 0244181 |
| 1464 | 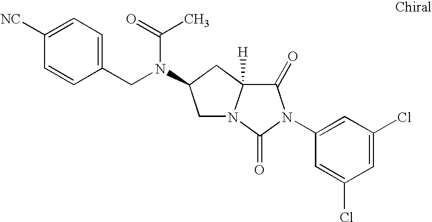 | Bristol- Myers Squibb | | WO 0244181 |
| 1465 | 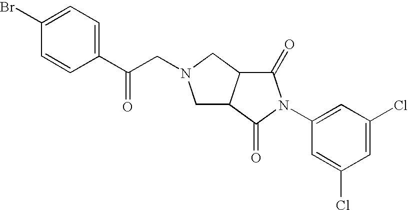 | Bristol- Myers Squibb | | WO 0244181 |
| 1466 | 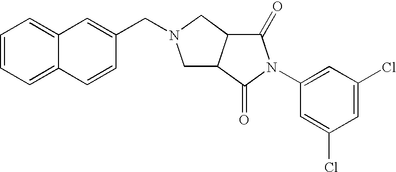 | Bristol- Myers Squibb | | WO 0244181 |
| 1467 | 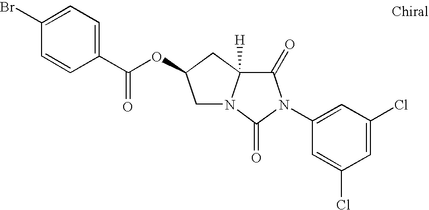 | Bristol- Myers Squibb | | WO 0244181 |
| 1468 | 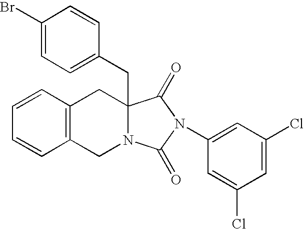 | Bristol- Myers Squibb | | WO 0244181 |
| 1469 | 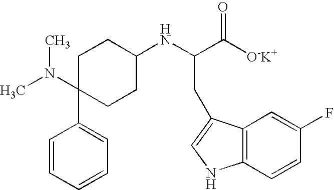 | Gruen- enthal | | WO 0290317 |

1. An isolated polypeptide of the SARS virus.

2. The polypeptide of claim 1, wherein the polypeptide is a Spike (S) polypeptide, an Env (E) polypeptide, a Membrane (M) polypeptide, a hemagglutinin-esterase polypeptide (HE), a nucleocapsid (N) polypeptide, a ORF1a polypeptide, a ORF1ab polypeptide, a proteolytic fragment of a ORF1a polypeptide, or a proteolytic fragment of a ORF1ab polypeptide.

3. The polypeptide of claim 1, wherein the polypeptide comprises an amino acid sequence selected from the group consisting of SEQ ID NOS: 6039, 7232, 9766, 9767, 9768, 9769, 9770, 9771, 9772, 9773, 9774, 9775, 9776, 9777, 9778, 9779, 6042, 6043, 6044, 6045, 6046, 6047, 6048, 6049, 6050 or 6052.

4. The polypeptide of claim 1, wherein the polypeptide comprises an amino acid sequence having >75% sequence identity to an amino acid sequence selected from the group consisting of SEQ ID NOS: 6042, 6043, 6044, 6045, 6046, 6047, 6048, 6049, 6050, 6052, 9766, 9767, 9768, 9769, 9770, 9771, 9772, 9773, 9774, 9775, 9776, 9777, 9778, 9779, 9997, 9998, 10149, 10316, 10338, 10339, 10340, 10341, 10342, 10532, 10533, 10571, 10572, 10573, 10574, 10575, 10576, 10577, 10578, 10579, 11561, 11562, 11618, 11619, 11620, 11627, 11630, 11633 & 11636.

5. The polypeptide of claim 1, wherein the polypeptide comprises a fragment of at least 10 consecutive amino acids of an amino acid sequence selected from the group consisting of SEQ ID NOS: 6042, 6043, 6044, 6045, 6046, 6047, 6048, 6049, 6050, 6052, 9766, 9767, 9768, 9769, 9770, 9771, 9772, 9773, 9774, 9775, 9776, 9777, 9778, 9779, 9997, 9998, 10149, 10316, 10338, 10339, 10340, 10341, 10342, 10532, 10533, 10571, 10572, 10573, 10574, 10575, 10576, 10577, 10578, 10579, 11552, 11561, 11562, 11618, 11619, 11620, 11627, 11630, 11633 & 11636.

6. A polypeptide comprising an amino acid sequence having >80% sequence identity to an amino acid sequence selected from the group consisting of SEQ ID NOS: 6042, 6043, 6044, 6045, 6046, 6047, 6048, 6049, 6050, 6052, 9766, 9767, 9768, 9769, 9770, 9771, 9772, 9773, 9774, 9775, 9776, 9777, 9778, 9779, 9997, 9998, 10149, 10316, 10338, 10339, 10340, 10341, 10342, 10532, 10533, 10571, 10572, 10573, 10574, 10575, 10576, 10577, 10578, 10579, 11552, 11561, 11562, 11618, 11619, 11620, 11627, 11630, 11633 & 11636.

7. A polypeptide comprising an amino acid sequence that comprises a fragment of at least 10 consecutive amino acids of an amino acid sequence selected from the group consisting SEQ ID NOS: 6042, 6043, 6044, 6045, 6046, 6047, 6048, 6049, 6050, 6052, 9766, 9767, 9768, 9769, 9770, 9771, 9772, 9773, 9774, 9775, 9776, 9777, 9778, 9779, 9997, 9998, 10149, 10316, 10338, 10339, 10340, 10341, 10342, 10532, 10533, 10571, 10572, 10573, 10574, 10575, 10576, 10577, 10578, 10579, 11552, 11561, 11562, 11618, 11619, 11620, 11627, 11630, 11633 & 11636.

8. A polypeptide comprising an amino acid sequence having >80% sequence identity to SEQ ID NO: 6042, and/or comprising an amino acid sequence that comprises a fragment of at least 10 consecutive amino acids of SEQ ID NO: 6042, wherein the polypeptide is in the form of a trimer.

9. Nucleic acid encoding the polypeptide of any one of claims 1 to 8.

10. Nucleic acid according to claim 9, comprising a nucleotide sequence selected from the group consisting of SEQ ID NOS: 7191, 7273, 7275, 7277, 7279, 7281, 7283, 7285, 7287, 7289, 7291, 7292, 7293, 9968, 10066, 10084, 10299, 10505, 11323, 11563, 11639 & 11640.

11. A polynucleotide comprising a nucleotide sequence having >80% sequence identity to the nucleic acid of claim 9 or claim 10.

12. A polynucleotide comprising a fragment of at least 10 consecutive nucleotides of the nucleic acid of claim 9 or claim 10.

13. Antibody that recognizes the polypeptide of any one of claim 1 to 8.

14. The antibody of claim 13, wherein said antibody recognizes the polypeptide comprising the amino acid sequence of SEQ ID NO: 6042 or a fragment thereof.

15. The antibody of claim 14, wherein said antibody recognizes the polypeptide comprising the amino acid sequence of SEQ ID NO: 6042 or a fragment thereof in trimeric form.

16. The antibody of claim 13, wherein the antibody is a monoclonal antibody.

17. The antibody of claim 13, wherein the antibody is a human antibody.

18. An immunoassay for detecting a SARS virus antigen in a sample, comprising the step of contacting the sample with the antibody of any one of claims 13 to 17.

19. An immunoassay for detecting an antibody against a SARS virus antigen in a sample, comprising the step of contacting the sample with the polypeptide of any one of claims 1 to 8.

20. A method of detecting an antibody against a SARS virus antigen in a sample comprising contacting said sample with the polypeptide of any one of claims 1 to 8, under conditions suitable for binding said polypeptide to said antibody, if present, and detecting the binding of said polypeptide to said antibody.

21. A method for detecting a SARS virus antigen in a sample comprising contacting said sample with the antibody of any one of claims 13 to 17, under conditions suitable for binding said antibody to said antigen, if present, and detecting the binding of said antibody to said antigen.

22. A vaccine for the treatment or prevention of severe acute respiratory syndrome (SARS), comprising an inactivated SARS virus, a killed SARS virus, an attenuated SARS virus, a split SARS virus preparation, or at least one purified SARS virus antigens.

23. The vaccine of claim 22, comprising a purified polypeptide according to any one of claims 1 to 8.

24. The vaccine of claim 22 or claim 23, wherein the antigen is a purified SARS virus antigen in the form of a VLP.

25. The vaccine of any one of claims 22 to 24, further comprising an adjuvant.

26. The vaccine of claim 25, wherein the adjuvant is an aluminium salt or is MF59.

27. The vaccine of any one of claims 22 to 26, comprising more than one SARS virus antigen.

28. The vaccine of claim 27, wherein the antigens are selected from S, E, N and M.

29. The vaccine of claim 22, comprising an inactivated SARS virus.

30. The vaccine of claim 29, wherein said virus is inactivated by chemical or physical means.

31. The vaccine of claim 30, wherein said inactivation comprises treatment of the virus with an effective amount of one or more of the following agents selected from the group consisting of detergents, formaldehyde, formalin, β-propriolactone, and UV light.

32. The vaccine of claim 30, wherein said inactivation comprises treatment of the virus with an effective amount of one or more of the following agents selected from the group consisting of methylene blue, psoralen and carboxyfullerene (C60).

33. The vaccine of claim 30, wherein said inactivation comprises treatment of the virus with an effective amount of one or more of the following agents selected from the group consisting of binary ethylamine, acetyl ethyleneimine and gamma irradiation.

34. The vaccine of claim 31, wherein said inactivation comprises treatment with β-propriolactone.

35. The vaccine of claim 34, wherein said β-propriolactone is used at a concentration of 0.01 to 0.5%.

36. The vaccine of claim 34, wherein said β-propriolactone is used at a concentration of 0.5 to 0.2%.

37. The vaccine of claim 34, wherein said β-propriolactone is used at a concentration of 0.025 to 0.1%.

38. A method of inactivating SARS virus comprising exposing the virus to an inactivation agent for 12 to 24 hours at refrigeration temperatures followed hydrolysis of any residual inactivating agent by elevating the temperature for three hours.

39. The method of claim 38, wherein the inactivation agent is β-propriolactone.

40. The method of claim 38, wherein the refrigeration temperature is between 0° C. and 8° C.

41. The method of claim 38, wherein the elevated temperature is between 33° C. and 41° C.

42. A method for making an inactivated SARS vaccine comprising:
a. innoculating a mammalian cell culture with SARS virus;
b. cultivating the infected cells;
c. harvesting SARS virus containing supernatant;
d. inactivating the SARS virus; and
e. purifying the inactivated SARS virus.

43. The method of claim 42, wherein said mammalian cell culture is derived from one or more of the cell types selected from the group consisting of fibroblast cells, endothelial cells, hepatocytes, keratinocytes, immune cells, mammary cells, smooth muscle cells, melanocyte cells, neural cells, prostate cells, renal cells, skeletal cells, liver cells, retinoblast cells and stromal cells.

44. The method of claim 42, wherein said mammalian cell culture is derived from a cell culture selected from the group consisting of human cells, non-human primate cells, HeLa cells, human diploid cells, fetal rhesus lung cells, human embryonic kidney cells, VERO cells, horse cells, cow cells, sheep cells, dog cells, cat cells or rodent cells.

45. The method of claim 42, wherein said mammalian cell culture is derived from VERO cells or fetal rhesus kidney cells.

46. The method of claim 42, wherein said mammalian cells are cultured in serum free media.

47. The method of claim 42, wherein said mammalian cells are cultured in protein free media.

48. The method of claim 42, wherein said inoculating step comprising absorbing the SARS virus onto the cell culture for 60 to 300 minutes.

49. The method of claim 42, wherein said inoculating step is conducted at 25° C. to 40° C.

50. The method of claim 42, wherein said purification step comprises one or more of the treatments selected from the group consisting of gradient centrifugation, ultracentrifugation, continuous-flow ultracentrifugation, chromatography, polyethylene glycol precipitation, and ammonium sulfate precipitation.

51. The method of claim 42, wherein said purification step comprises one or more of the treatments selected from the group consisting of ultrafiltration and dialfiltration.

52. The method of claim 50, wherein said chromatography treatment includes one or more of the chromatography treatments selected from the group consisting of ion exchange chromatography, size exclusion chromatography, and liquid affinity chromatography.

53. The method of claim 52, wherein said chromatography treatment includes use of one more chromatographic resins selected from the group consisting of an an anionic resin and a cationic resin.

54. The method of claim 52, wherein the ion exchange chromatography treatment includes a first step using a strong anion exchange resin and a second step using a strong cation exchange resin.

55. The method of claim 50, wherein said gradient centrifugation purification step comprises density gradient centrifugation.

56. The method of claim 42, wherein said purification step comprises a first step of chromatography purification and a second step of gradient centrifugation.

57. The method of claim 56, wherein said first chromatography purification step comprises liquid affinity chromatography.

58. The method of claim 56, wherein said second gradient centrifugation step comprises density gradient centrifugation.

59. A single-stranded oligonucleotide comprising a nucleotide sequence selected from the group consisting of SEQ ID NOS: 21-6020, 6076-6568, 6586-6587, 7292-7301, 7325-7328, 7332-7352, 7353-7385, 10235-10298, 10352-10504, 10580-11322 and 11325-11551.

60. A single-stranded oligonucleotide comprising the complement of the oligonucleotide of claim 59.

61. The oligonucleotide of claim 59 or claim 60, comprising 10-30 nucleotides.

62. The oligonucleotide of claim 61, comprising the nucleotide sequence of SEQ ID NO: 7292, SEQ ID NO: 7293, the complement of SEQ ID NO: 7292 or the complement of SEQ ID NO: 7293.

63. A kit comprising primers for amplifying a template sequence contained within a SARS virus nucleic acid target, the kit comprising a first primer and a second primer, wherein the first primer comprises a sequence substantially complementary to a portion of said template sequence and the second primer comprises a sequence substantially complementary to a portion of the complement of said template sequence, wherein the sequences within said primers which have substantial complementarity define the termini of the template sequence to be amplified.

64. The kit of claim 63, wherein the template sequence is contained within SEQ ID NO: 1 and/or SEQ ID NO: 2.

65. The kit of claim 63 or claim 64, wherein the first primer comprises a fragment of 8 or more nucleotides of SEQ ID NO: 1, and the second primer comprises a fragment of 8 or more nucleotides of the complement of SEQ ID NO: 1.

66. The kit of claim 63 or claim 64, wherein the first primer comprises a fragment of 8 or more nucleotides of SEQ ID NO: 2, and the second primer comprises a fragment of 8 or more nucleotides of the complement of SEQ ID NO: 2.

67. The kit of claim 63, wherein the first primer is an oligonucleotide according to any one of claims 59 to 62 and the second primer is an oligonucleotide according to any of claims 59 to 62.

68. The kit of any one of claims 63 to 67, further comprising a labeled probe that comprises either a fragment of 8 or more nucleotides of SEQ ID NO: 1 and/or SEQ ID NO: 2, or the complement of said fragment, which fragment is located within the template sequence.

69. The kit of any one of claims 63 to 68, wherein the first primer and/or the second primer comprises a nucleotide sequence selected from the group consisting of SEQ ID NOS: 21-6020, 6076-6568, 6586-6587, 7292-7301, 7325-7328, 7332-7352, 7353-7385, 10235-10298, 10352-10504, 10580-11322 and 11325-11551.

70. The kit of any one of claims 63 to 68, wherein the first primer and/or the second primer comprises the complement of a nucleotide sequence selected from the group consisting of SEQ ID NOS: 21-6020, 6076-6568, 6586-6587, 7292-7301, 7325-7328, 7332-7352, 7353-7385, 10235-10298, 10352-10504, 10580-11322 and 11325-11551.

71. A method of detecting the presence of SARS virus in a sample comprising providing a sample suspected of containing a SARS virus nucleic acid target, amplifying a template sequence contained within said SARS virus nucleic acid target with the kit of any one of claims 63 to 70, and detecting the amplified template sequence, wherein the presence of the amplified template sequence indicates the presence of SARS virus in said sample.

72. The method of claim 71, wherein said amplifying is accomplished using polymerase chain reaction, transcription mediated amplification, reverse transcription PCR, ligase chain reaction, strand displacement amplification or nucleic acid sequence-based amplification.

73. A double-stranded RNA molecule with a length from about 10 to about 30 nucleotides which is able to inactivate the SARS coronavirus in a mammalian cell.

74. The double-stranded RNA of claim 73, wherein the sequence of one of the strands is at least 90% identical to a target sequence, wherein the target sequence is a fragment of SEQ ID NO: 1 and/or SEQ ID NO: 2.

75. The double-stranded RNA of claim 73 or claim 74, wherein the target sequence comprising a nucleotide sequence selected from the group consisting of SEQ ID NOS: 7292, 7293, 7294, 7295, 7296, 7297, 7298, 7299, 7300 and 7301.

76. The double-stranded RNA of any one of claims 73 to 75, comprising at least one modified nucleotide.

77. A method for treating a patient suffering from SARS, comprising: administering to the patient a therapeutically effective dose of a molecule of less than 1000 g/mol.

78. The method of claim 77, wherein the molecule has an aromatic region and greater than one heteroatom selected from O, S, or N.

79. A method for treating a patient suffering from SARS, comprising: administering to the patient a therapeutically effective dose of a compound selected from: a nucleoside analog, a peptoid, an oligopeptide, a polypeptide a protease inhibitor, a 3C-like protease inhibitor, a papain-like protease inhibitor, or an inhibitor of an RNA dependent RNA polymerase.

80. A method for treating a patient suffering from SARS, comprising: administering to the patient a steroidal anti-inflammatory drug in combination with at least one antiviral compound.

81. A method for treating a patient suffering from SARS, comprising: administering to the patient a therapeutically effective dose of a compound selected from: acyclovir, gancyclovir, vidarabidine, foscamet, cidofovir, amantidine, ribavirin, trifluorothymidine, zidovudine, didanosine, zalcitabine, an antiviral compound listed in Table 1; an antiviral compound listed in Table 2; or an interferon.

82. The method of claim 81, wherein the interferon is an interferon-α or an interferon-β.

83. The method of any one of claims 77 to 82, wherein the molecule or compound is delivered by inhalation.

84. A method of identifying a therapeutically active agent comprising the steps of: (a) contacting a therapeutically active agent with a cell infected with the SARS virus; (b) measuring attenuation of a SARS related enzyme.

85. A viral vector or particle for in vivo delivery of a nucleic acid of claim 9 or claim 10.

86. The viral vector of claim 85, wherein the vector is an adenovirus vector, a poxvirus vector or an alphavirus vector.

87. An alphavirus replicon particle comprising one or more SARS viral antigens.

88. The replicon particle of claim 87, wherein said SARS viral antigen is a spike protein.

89. The replicon particle of claim 87, wherein said particle comprises a replicon derived from Venezuelan Equine Encephalitis (VEE) and further comprises an envelope derived from Sindbus virus (SIN) or Semliki Forest Virus (SFV).

90. A vaccine comprising one or more SARS virus antigens and one or more respiratory virus antigens.

91. The vaccine of claim 90, wherein said respiratory virus antigens are selected from the group consisting of influenza virus, human rhinovirus (HRV), parainfluenza virus (PIV), respiratory syncytial virus (RSV), adenovirus, metapneumovirus, and rhinovirus.

92. The vaccine of claim 91, wherein said respiratory virus antigen is from influenza virus.

93. The vaccine of claim 90, wherein said respiratory virus antigen is from a coronavirus other than the SARS virus.

94. A polypeptide comprising an immunogenic, surface exposed fragment of the amino acid sequence SEQ ID NO: 6042.

95. The polypeptide of claim 94, wherein said fragment does not include the last 50 amino acids of the C-terminus of SEQ ID NO: 6042.

96. The polypeptide of claim 94, wherein said fragment does not include a transdomain region of SEQ ID NO: 6042.

97. The polypeptide of claim 94, wherein said fragment does not include a C-terminus cytoplasmic domain of SEQ ID NO: 6042.

98. The polypeptide of claim 94, wherein said fragment does not include a N-terminus signal sequence.

99. An isolated polynucleotide comprising a nucleic acid sequence selected from the group consisting of SEQ ID NOS: 9968 and 10066.

100. The polynucleotide of claim 99, wherein the polynucleotide comprising a nucleic acid sequence having >80% sequence identity to a polynucleotide sequence selected from the group consisting of SEQ ID NOS: 9968 and 10066.

101. An isolated polynucleotide comprising a fragment of at least 15 consecutive nucleic acids of a nucleic acid sequence selected from the group consisting of SEQ ID NOS: 9968 and 10066 and wherein said fragment does not consist entirely of SEQ ID NO: 10033.

102. An isolated polypeptide comprising an amino acid sequence encoded by any one of claims 99-101.

103. The polypeptide of claim 102, comprising an amino acid sequence selected from the group consisting of SEQ ID NOS: 9969-10032, 10067, and 10015.

104. The polypeptide of claim 103, wherein the amino acid sequence is selected from the group consisting of SEQ ID NOS: 9997, 9998 and 10015.

105. An expression construct for recombinant expression of a SARS virus spike protein wherein said construct comprises a nucleic acid sequence selected from the group consisting of SEQ ID NOS: 6578-6583.

106. A mammalian cell line stably expressing a SARS viral antigen.

107. The cell line of claim 106, wherein said cell line is a Chinese Hamster Ovary (CHO) cell.

108. The cell line of claim 106, wherein the SARS viral antigen is a spike protein or fragment thereof.

109. The cell line of claim 106, wherein the spike protein is truncated to remove the transmembrane sequence.

110. A method of identifying a therapeutically active agent comprising the steps of: (a) contacting a therapeutically active agent with a buffer comprising SARS enzyme; and (b) measuring attenuation of the SARS enzyme.

111. The method of claim 110 wherein the SARS enzyme is a SARS protease.

112. The method of claim 111 wherein the buffer further comprises a peptide with a SARS protease cleave site.

113. The method of claim 110 wherein the measurement is made by the measurement of fluorescence.

114. A vaccine of one of claims 22 to 37, and 90 to 93 further comprising an adjuvant.

115. The vaccine of claim 114 wherein the adjuvant is a SMIP.

116. The vaccine of claim 115 wherein the SMIP compound is selected from the group consisting of an acylpiperazine, a tryptanthrin, an indoledione, a tetrahydroisoquinoline, a benzocyclodione, an amino azavinyl compound, a thiosemicarbazone, a lactam, an aminobenzimidazole quinolinone, a hydropthalamide, a benzophenone, an isoxazole, a sterol, a quinazolinone, a pyrole, an anthraquinone, a quinoxaline, a triazine, an benzazole, and a pyrazolopyrimidine, or a pharmaceutically acceptable salt, ester, or prodrug thereof.

117. A method of vaccinating a subject comprising administering a vaccine of one of claims 22 to 37, and 90 to 93.

118. The method of claim 117 further comprising administering a SMIP.

119. A method for treating a patient of one of claims 77 to 82 further comprising administering at least one SMEP compound.

120. A method for treating a patient of one of claims 77 to 82 further comprising administering at least one SMIS compound.
